SEQUENCE LISTING (1 sequence)

SEQ ID NO: 1 - protein, 510 aa, Saccharomyces cerevisiae
MPTSGTTIELIDDQFPKDDSASSGIVDEVDLTEANILATGLNKKAPRIVNGFGSLMGSKEMVSVEFDKKGNEKKSNLDRLLEKDNQEKEEAKTKIHISEQPWTLNNWHQHLNWLNMVLVCGMPMIGWYFALSGKVPLHLNVFLFSVFYYAVGGVSITAGYHRLWSHRSYSAHWPLRLFYAIFGCASVEGSAKWWGHSHRIHHRYTDTLRDPYDARRGLWYSHMGWMLLKPNPKYKARADITDMTDDWTIRFQHRHYILLMLLTAFVIPTLICGYFFNDYMGGLIYAGFIRVFVIQQATFCINSLAHYIGTQPFDDRRTPRDNWITAIVTFGEGYHNFHHEFPTDYRNAIKWYQYDPTKVIIYLTSLVGLAYDLKKFSQNAIEEALIQQEQKKINKKKAKINWGPVLTDLPMWDKQTFLAKSKENKGLVIISGIVHDVSGYISEHPGGETLIKTALGKDATKAFSGGVYRHSNAAQNVLADMRVAVIKESKNSAIRMASKRGEIYETGKFF

(19) United States
(12) Patent Application Publication
(10) Pub. No.: US 2020/0222400 A1
(43) Pub. Date: Jul. 16, 2020

(54) METHODS FOR THE TREATMENT OF NEUROLOGICAL DISORDERS

(71) Applicant: Yumanity Therapeutics, Inc., Cambridge, MA (US)

(72) Inventors: Kenneth RHODES, Belmont, MA (US); Bertrand LE BOURDONNEC, Northborough, MA (US); Robert SCANNEVIN, Hopkinton, MA (US)

(21) Appl. No.: 16/744,473

(22) Filed: Jan. 16, 2020

Related U.S. Application Data
(60) Provisional application No. 62/793,018, filed on Jan. 16, 2019.

(51) Int. Cl. A61K 31/501; A61K 31/454; A61K 31/445; A61K 31/202; A61K 31/343; A61K 31/7072; A61P 25/28

(52) U.S. Cl. CPC A61K 31/501; A61K 31/454; A61K 31/445; A61P 25/28; A61K 31/343; A61K 31/7072; A61K 31/202

(57) ABSTRACT

The present disclosure provides compounds and methods useful in the treatment of neurological disorders. The compounds of the invention, alone or in combination with other pharmaceutically active agents, can be used for treating or preventing neurological disorders.

### BACKGROUND OF THE INVENTION

An incomplete understanding of the molecular perturbations that cause disease, as well as a limited arsenal of robust model systems, has contributed to a failure to generate successful disease-modifying therapies against common and progressive neurological disorders, such as Parkinson's Disease (PD) and Alzheimer's Disease (AD). Progress is being made on many fronts to find agents that can arrest the progress of these disorders. However, the present therapies for most, if not all, of these diseases provide very little relief. Accordingly, a need exists to develop therapies that can alter the course of neurological diseases (e.g., neurodegenerative diseases). More generally, a need exists for better methods and compositions for the treatment of neurological disorders in order to improve the quality of the lives of those afflicted by such diseases.

Fatty acid synthase (FASN) catalyzes the conversion of malnoyl-CoA and acetyl-CoA to the saturated C16 fatty acid palmitate. Palmitate is subsequently used as the precursor for the synthesis of complex lipid molecules. The present inventors have discovered that inhibition of FASN is capable of suppressing toxicity in cells related to protein misfolding and/or aggregation. Accordingly, inhibition of FASN may provide new methods for the treatment of diseases and disorders related to toxicity caused by protein misfolding and/or aggregation.

### SUMMARY OF THE INVENTION

Described herein are compounds that modulate the activity of fatty acid synthase (FASN), pharmaceutical compositions including such compounds, and methods of utilizing such compounds and compositions for modulating the activity of FASN for the treatment of diseases and disorders related to toxicity caused by proteins, such as toxicity related to misfolding and/or aggregation of proteins. In some embodiments, the disease or disorder is a neurological disorder.

In one aspect, the invention features a method of treating a neurological disorder in a subject in need thereof, the method including administering a FASN inhibitor in an amount sufficient to suppress toxicity in a cell related to protein misfolding and/or aggregation.

In another aspect, the invention features a method of suppressing toxicity in a cell related to protein misfolding and/or aggregation in a subject, the method including contacting a cell with a FASN inhibitor.

In some embodiments, the toxicity in the cell is related to protein aggregation related to misfolding of a protein. In some embodiments, the toxicity in the cell is related to misfolding and/or aggregation of α-synuclein or apolipoprotein E4 (ApoE4). In some embodiments, the cell is a neural cell, e.g., a neuron or glial cell.

In another aspect, the invention features a method of treating a neurological disorder in a subject in need thereof, the method including: (a) determining the expression level of α-synuclein, ApoE4, or an undesired form thereof in the subject; (b) administering an effective amount of a FASN inhibitor to the subject if the level of α-synuclein, ApoE4, and/or the undesired form thereof is greater than a predetermined level.

In another aspect, the invention features a method of treating a neurological disease in a subject in need thereof, wherein the subject has an elevated level, or is predicted to have an elevated level of α-synuclein, ApoE4, or an undesired form thereof the method including administering an effective amount of a FASN inhibitor to the subject.

In some embodiments, the subject is predicted to have an elevated level of α-synuclein, ApoE4, and/or an undesired form thereof based on genetic markers. In some embodiments, the subject carries one or two copies of the ApoE4 allele.

In some embodiments, the FASN inhibitor is a compound of any one of Formula I-LV, or any one of compounds 1-2282.

In some embodiments, the FASN inhibitor is a compound of Formula (I):

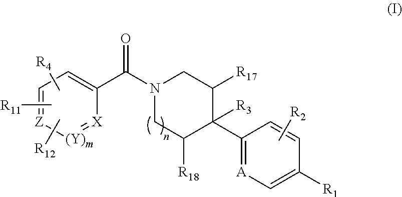

or a pharmaceutically acceptable salt thereof, wherein: X, Y, and Z are each, independently, CR or NR′, wherein R is hydrogen or C₁₋₆ alkyl and R′ is hydrogen, C₁₋₆ alkyl, or absent; A is CH or N; R₁ is hydrogen, cyano, halo, C₁₋₆ alkyl, C₁₋₆ alkoxy, —C(═O)NR₁₃R₁₄, —(CH₂)_{q}C(═O)NR₁₃R₁₄, CF₃, —OCF₃, or —S(═O)₂R₂₀; q is 0, 1, 2, 3, or 4; R₂₀ is hydrogen or C₁₋₆ alkyl, C₁₋₆ alkoxy, or —NR₁₃R₁₄; R₂ is hydrogen, halo, C₁₋₆ alkoxy, C₁₋₆ alkyl, or R₂ and R₃ taken together with the atoms to which they are attached form a 5-membered heterocyclyl; R₃ is hydrogen, hydroxyl, halo, C₁₋₆ alkyl, C₁₋₆ alkoxy, or R₂ and R₃ taken together with the atoms to which they are attached form a 5-membered heterocyclyl; R₄ is hydrogen, heteroaryl, heterocyclyl, —C(═O)NR₅R₆, —NR₇C(═O)R₈, —NR₉R₁₀, C₁₋₆ alkyl, C₁₋₆ alkoxy, —S(═O)₂R₂₀, or R₄ and R₁₁ taken together with the atoms to which they are attached join together to form a heteroaryl; R₁₁ is hydrogen, halo, cyano, C₁₋₆ alkyl, C₁₋₆ alkoxy, —NR₁₃R₁₄, CF₃, —OCF₃, —S(═O)₂R₂₀, R₄ and R₁₁ taken together with the atoms to which they are attached join together to form a heteroaryl, or R₁₁ and R₁₂ taken together with the atoms to which they are attached join together to form a heteroaryl; R₁₂ is hydrogen, halo, cyano, C₁₋₆ alkyl, C₁₋₆ alkoxy, —NR₁₃R₁₄, CF₃, —OCF₃, —S(═O)₂R₂₀, or R₁₁ and R₁₂ taken together with the atoms to which they are attached join together to form a heteroaryl; R₅, R₆, R₇, R₈, R₉, R₁₀, R₁₃, and R₁₄ are each, independently, hydrogen, C₁₋₆ alkyl, cycloalkyl, aryl, heterocyclyl, heteroaryl, hydroxyalkyl, alkylamino, —NR₁₅R₁₆, or —S(═O)₂R₂₀; R₁₅ and R₁₆ are each, independently, hydrogen, C₁₋₆ alkyl, cycloalkyl, aryl, heterocyclyl, heteroaryl, or alkylamino; R₁₇ and R₁₈ are each, independently, hydrogen or alkyl or can optionally join together to form a bond; n is 1 or 2; and m is 0 or 1.

In some embodiments of Formula (I) R₃ is F. In some embodiments of Formula (I), A is CH. In some embodiments of Formula (I), A is N. In some embodiments of Formula (I), X, Y, and Z are NR′. In some embodiments of Formula (I), R₄ is heteroaryl, heterocyclyl, —C(═O)NR₅R₆, —NR₇C(═O)R₈, —NR₉R₁₀, C₁₋₆ alkyl, C₁₋₆ alkoxy, or R₄ and R₁₁ taken together with the atoms to which they are attached join together to form a heteroaryl. In some embodiments of Formula (I), R₅ is hydrogen and R₆ is aryl or heteroaryl. In some embodiments of Formula (I), the compound has a structure of one of the following:

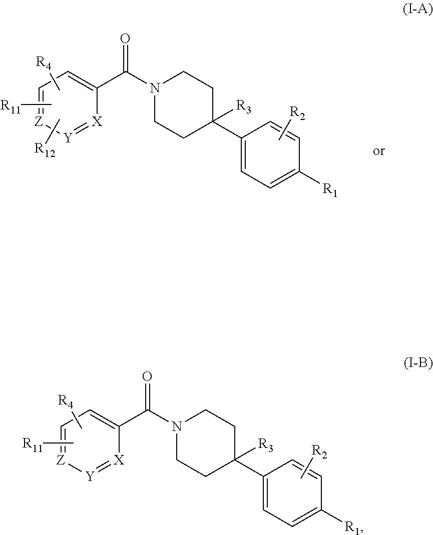

or a pharmaceutically acceptable salt thereof, wherein: X, Y, and Z are each independently CR or NR′, wherein R is hydrogen or C₁₋₆ alkyl and R′ is hydrogen, C₁₋₆ alkyl, or absent; R₁ is hydrogen, cyano, halo, C₁₋₆ alkyl, C₁₋₆ alkoxy, —C(═O)NR₁₃R₁₄, —(CH₂)_{q}C(═O)NR₁₃R₁₄, CF₃, —OCF₃, or —S(═O)₂R₂₀; q is 0, 1, 2, 3, or 4; R₂₀ is hydrogen or C₁₋₆ alkyl, C₁₋₆ alkoxy, or —NR₁₃R₁₄; R₂ is hydrogen, halo, C₁₋₆ alkoxy, C₁₋₆ alkyl, or R₂ and R₃ taken together with the atoms to which they are attached form a 5-membered heterocyclyl; R₃ is hydrogen, hydroxyl, halo, C₁₋₆ alkyl, C₁₋₆ alkoxy, or R₂ and R₃ taken together with the atoms to which they are attached form a 5-membered heterocyclyl; R₄ is hydrogen, heteroaryl, heterocyclyl, C(═O)NR₅R₆, —NR₇C(═O)R₈, —NR₉R₁₀, C₁₋₆ alkyl, C₁₋₆ alkoxy, —S(═O)₂R₂₀, or R₄ and R₁₁ taken together with the atoms to which they are attached join together to form a heteroaryl; R₁₁ is hydrogen, halo, cyano, C₁₋₆ alkyl, C₁₋₆ alkoxy, —NR₁₃R₁₄, CF₃, —OCF₃, —S(═O)₂R₂₀, R₄ and R₁₁ taken together with the atoms to which they are attached join together to form a heteroaryl, or R₁₁ and R₁₂ taken together with the atoms to which they are attached join together to form a heteroaryl; R₁₂ is hydrogen, halo, cyano, C₁₋₆ alkyl, C₁₋₆ alkoxy, —NR₁₃R₁₄, CF₃, —OCF₃, —S(═O)₂R₂₀, or R₁₁ and R₁₂ taken together with the atoms to which they are attached join together to form a heteroaryl; R₅, R₆, R₇, R₈, R₉R₁₀, R₁₃, and R₁₄ are each independently hydrogen, C₁₋₆ alkyl, cycloalkyl, aryl, heterocyclyl, heteroaryl, hydroxyalkyl, alkylamino, —NR₁₅R₁₆, or —S(═O)₂R₂₀; R₁₅ and R₁₆ are each independently hydrogen, C₁₋₆ alkyl, cycloalkyl, aryl, heterocyclyl, heteroaryl, or alkylamino; and R₁₇ and R₁₈ are each independently hydrogen or alkyl or can optionally join together to form a bond.

In some embodiments of Formula (I), the compound has structure of one of the following:

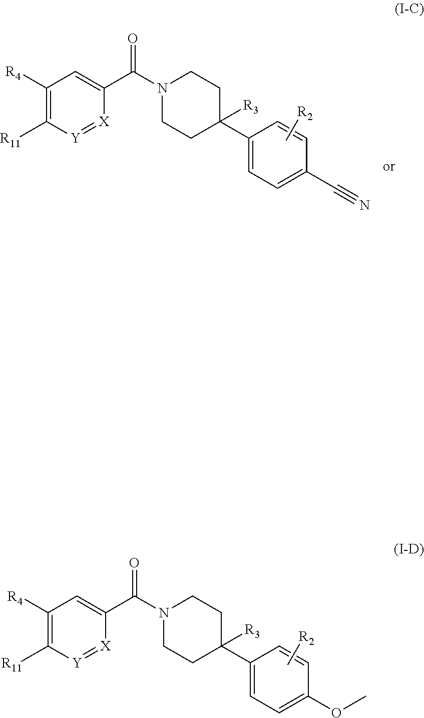

or a pharmaceutically acceptable salt thereof, wherein: X, Y, and Z are each independently CR or NR′, wherein R is hydrogen or C₁₋₆ alkyl and R′ is hydrogen, C₁₋₆ alkyl, or absent; R₂ is hydrogen, halo, C₁₋₆ alkoxy, C₁₋₆ alkyl, or R₂ and R₃ taken together with the atoms to which they are attached form a 5-membered heterocyclyl; R₃ is hydrogen, hydroxyl, halo, C₁₋₆ alkyl, C₁₋₆ alkoxy, or R₂ and R₃ taken together with the atoms to which they are attached form a 5-membered heterocyclyl; R₄ is hydrogen, heteroaryl, heterocyclyl, —C(═O)NR₅R₆, —NR₇C(═O)R₈, —NR₉R₁₀, C₁₋₆ alkyl, C₁₋₆ alkoxy, —S(═O)₂R₂₀, or R₄ and R₁₁ taken together with the atoms to which they are attached join together to form a heteroaryl; R₂₀ is hydrogen or C₁₋₆ alkyl, C₁₋₆ alkoxy, or —NR₁₃R₁₄; R₁₁ is hydrogen, halo, cyano, C₁₋₆ alkyl, C₁₋₆ alkoxy, NR₁₃R₁₄, CF₃, —OCF₃, —S(═O)₂R₂₀, or R₄ and R₁₁ taken together with the atoms to which they are attached join together to form a heteroaryl; R₅, R₆, R₇, R₈, R₉, and R₁₀ are each independently H, C₁₋₆ alkyl, cycloalkyl, aryl, heterocyclyl, heteroaryl, alkylamino, or —NR₁₅R₁₆; and R₁₅ and R₁₆ are each independently H, C₁₋₆ alkyl, cycloalkyl, aryl, heterocyclyl, heteroaryl, or alkylamino.

In some embodiments of Formula (I), the compound has structure of one of the following:

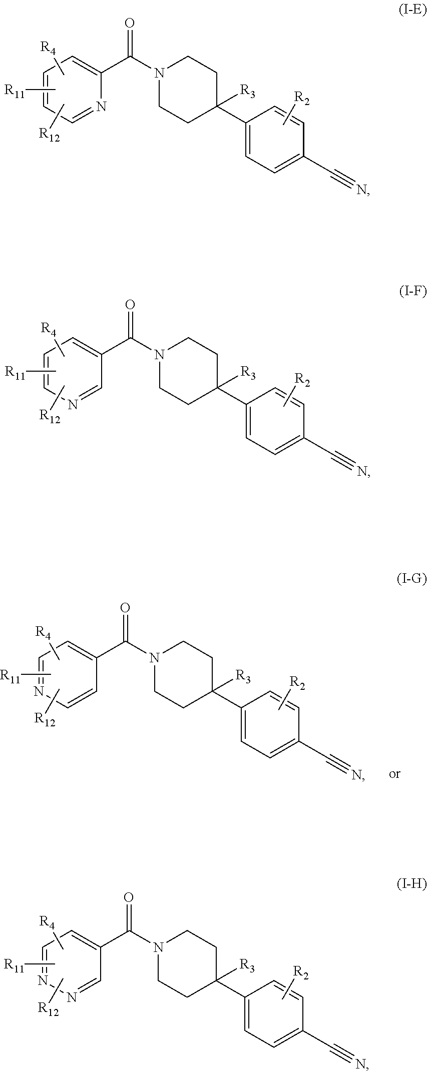

or a pharmaceutically acceptable salt thereof, wherein: R₂ is hydrogen, halo, C₁₋₆ alkoxy, C₁₋₆ alkyl, or R₂ and R₃ taken together with the atoms to which they are attached form a 5-membered heterocyclyl; R₃ is hydrogen, hydroxyl, halo, C₁₋₆ alkyl, C₁₋₆ alkoxy, or R₂ and R₃ taken together with the atoms to which they are attached form a 5-membered heterocyclyl; R₄ is hydrogen, heteroaryl, heterocyclyl, —C(═O)NR₅R₆, —NR₇C(═O)R₈, —NR₉R₁₀, C₁₋₆ alkyl, C₁₋₆ alkoxy, —S(═O)₂R₂₀, or R₄ and R₁₁ taken together with the atoms to which they are attached join together to form a heteroaryl; R₂₀ is hydrogen or C₁₋₆ alkyl, C₁₋₆ alkoxy, or —NR₁₃R₁₄; R₁₁ is hydrogen, halo, cyano, 1-6 alkyl, C₁₋₆ alkoxy, —NR₁₃R₁₄, F₃, —OCF₃, —S(═O)₂R₂₀, R₄ and R₁₁ taken together with the atoms to which they are attached join together to form a heteroaryl, or R₁₁ and R₁₂ taken together with the atoms to which they are attached join together to form a heteroaryl; R₁₂ is hydrogen, halo, cyano, C₁₋₆ alkyl, C₁₋₆ alkoxy, —NR₁₃R₁₄, CF₃, —OCF₃, —S(═O)₂R₂₀, or R₁₁ and R₁₂ taken together with the atoms to which they are attached join together to form a heteroaryl; R₅, R₆, R₇, R₈, R₉, R₁₀, R₁₃, and R₁₄ are each independently H, C₁₋₆ alkyl, cycloalkyl, aryl, heterocyclyl, heteroaryl, alkylamino, or —NR₁₅R₁₆; and R₁₅ and R₁₆ are each independently H, C₁₋₆ alkyl, cycloalkyl, aryl, heterocyclyl, heteroaryl, or alkylamino.

In some embodiments of Formula (I), the FASN inhibitor is one of the following:

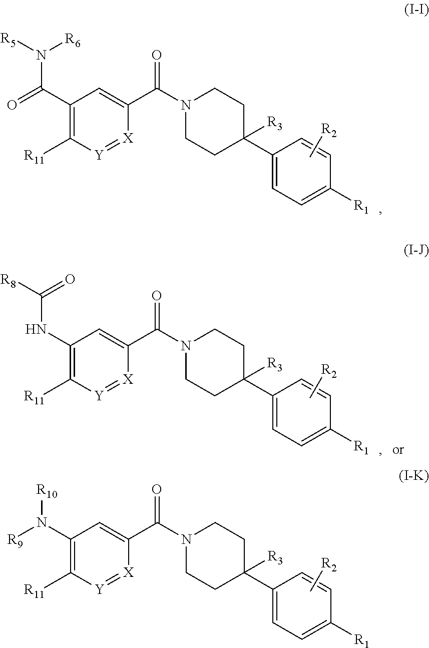

or a pharmaceutically acceptable salt thereof, wherein: X and Y are each independently CR or NR′, wherein R is H or C₁₋₆ alkyl and R′ is H, C₁₋₆ alkyl, or absent; R₁ is hydrogen, cyano, halo, C₁₋₆ alkyl, C₁₋₆ alkoxy, —C(═O)NR₁₃R₁₄, —(CH₂)_{q}C(═O)NR₁₃R₁₄, CF₃, —OCF₃, or —S(═O)₂R₂₀; q is 0, 1, 2, 3, or 4; R₂₀ is hydrogen or C₁₋₆ alkyl, C₁₋₆ alkoxy, or —NR₁₃R₁₄; R₂ is hydrogen, halo, C₁₋₆ alkoxy, C₁₋₆ alkyl, or R₂ and R₃ taken together with the atoms to which they are attached form a 5-membered heterocyclyl; R₃ is hydrogen, hydroxyl, halo, C₁₋₆ alkyl, C₁₋₆ alkoxy, or R₂ and R₃ taken together with the atoms to which they are attached form a 5-membered heterocyclyl; R₁₁ is hydrogen, halo, cyano, C₁₋₆ alkyl, C₁₋₆ alkoxy, —NR₁₃R₁₄, CF₃, —OCF₃, or —S(═O)₂R₂₀; R₅, R₆, R₇, R₈, R₉, and R₁₀ are each independently H, C₁₋₆ alkyl, cycloalkyl, aryl, heterocyclyl, heteroaryl, alkylamino, or —NR₁₅R₁₆; and R₁₅ and R₁₆ are each independently H, C₁₋₆ alkyl, cycloalkyl, aryl, heterocyclyl, heteroaryl, or alkylamino.

In some embodiments of Formula (I), the compound as the structure of one of the following:

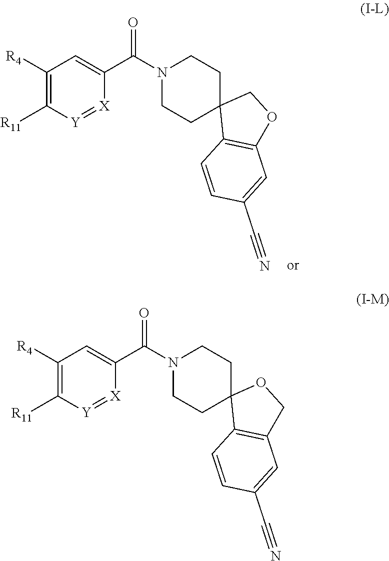

or a pharmaceutically acceptable salt thereof, wherein: X and Y are each independently CR or NR′, wherein R is H or C₁₋₆ alkyl and R′ is H, C₁₋₆ alkyl, or absent; R₄ is hydrogen, heteroaryl, heterocyclyl, —C(═O)NR₅R₆, —NR₇C(═O)R₈, —NR₉R₁₀, C₁₋₆ alkyl, C₁₋₆ alkoxy, —S(═O)₂R₂₀, or R₄ and R₁₁ taken together with the atoms to which they are attached join together to form a heteroaryl; R₂₀ is hydrogen or C₁₋₆ alkyl, C₁₋₆ alkoxy, or —NR₁₃R₁₄; R₁₁ is hydrogen, halo, cyano, C₁₋₆ alkyl, C₁₋₆ alkoxy, —NR₁₃R₁₄, CF₃, —OCF₃, S(═O)₂R₂₀, or R₄ and R₁₁ taken together with the atoms to which they are attached join together to form a heteroaryl; R₅, R₆, R₇, R₈, R₉, and R₁₀ are each independently H, C₁₋₆ alkyl, cycloalkyl, aryl, heterocyclyl, heteroaryl, alkylamino, or —NR₁₅R₁₆; and R₁₅ and R₁₆ are each independently H, C₁₋₆ alkyl, cycloalkyl, aryl, heterocyclyl, heteroaryl, or alkylamino.

In some embodiments of Formula (I), the FASN inhibitor is one of the following:

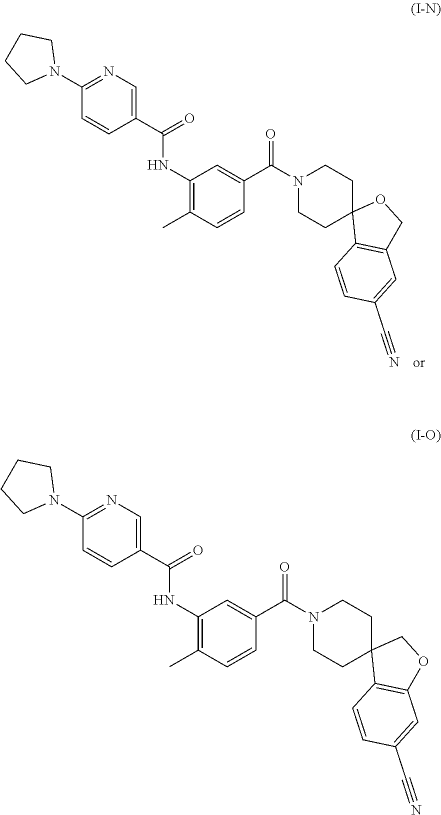 [SMILES: Cc1ccc(C(=O)N2CCC3(CC2)OCc2cc(C#N)ccc23)cc1NC(=O)c1ccc(N2CCCC2)nc1 ; Cc1ccc(C(=O)N2CCC3(CC2)COc2cc(C#N)ccc23)cc1NC(=O)c1ccc(N2CCCC2)nc1]

or a pharmaceutically acceptable salt thereof.

In some embodiments of Formula (I), the compound has the structure:

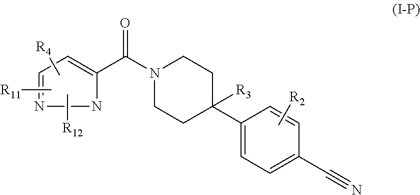

or a pharmaceutically acceptable salt thereof, wherein: R₂ is hydrogen, halo, C₁₋₆ alkoxy, C₁₋₆ alkyl, or R₂ and R₃ taken together with the atoms to which they are attached form a 5-membered heterocyclyl; R₃ is hydrogen, hydroxyl, halo, C₁₋₆ alkyl, C₁₋₆ alkoxy, or R₂ and R₃ taken together with the atoms to which they are attached form a 5-membered heterocyclyl; R₄ is hydrogen, heteroaryl, heterocyclyl, —C(═O)NR₅R₆, —NR₇C(═O)R₈, —NR₉R₁₀, C₁₋₆ alkyl, C₁₋₆ alkoxy, —S(═O)₂R₂₀, or R₄ and R₁₁ taken together with the atoms to which they are attached join together to form a heteroaryl; R₂₀ is hydrogen or C₁₋₆ alkyl, C₁₋₆ alkoxy, or —NR₁₃R₁₄; R₁₁ is hydrogen, halo, cyano, C₁₋₆ alkyl, C₁₋₆ alkoxy, —NR₁₃R₁₄, CF₃, —OCF₃, —S(═O)₂R₂₀, R₄ and R₁₁ taken together with the atoms to which they are attached join together to form a heteroaryl, or R₁₁ and R₁₂ taken together with the atoms to which they are attached join together to form a heteroaryl; R₁₂ is hydrogen, halo, cyano, C₁₋₆ alkyl, C₁₋₆ alkoxy, —NR₁₃R₁₄, CF₃, —OCF₃, —S(═O)₂R₂₀, or R₁₁ and R₁₂ taken together with the atoms to which they are attached join together to form a heteroaryl; R₅, R₆, R₇, R₈, R₉, R₁₀, R₁₃, and R₁₄ are each independently H, C₁₋₆ alkyl, cycloalkyl, aryl, heterocyclyl, heteroaryl, alkylamino, or —NR₁₅R₁₆; and R₁₅ and R₁₆ are each independently H, C₁₋₆ alkyl, cycloalkyl, aryl, heterocyclyl, heteroaryl, or alkylamino.

In some embodiments of Formula (I), the FASN inhibitor has the structure of one of the following:

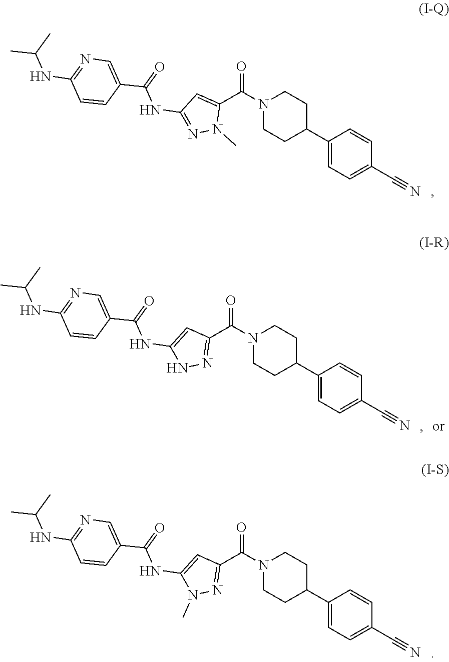 [SMILES: CC(C)Nc1ccc(C(=O)Nc2cc(C(=O)N3CCC(c4ccc(C#N)cc4)CC3)n(C)n2)cn1 ; CC(C)Nc1ccc(C(=O)Nc2cc(C(=O)N3CCC(c4ccc(C#N)cc4)CC3)n[nH]2)cn1 ; CC(C)Nc1ccc(C(=O)Nc2cc(C(=O)N3CCC(c4ccc(C#N)cc4)CC3)nn2C)cn1]

or a pharmaceutically acceptable salt thereof.

In some embodiments of Formula (I), the compound has the structure:

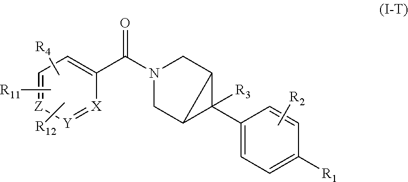

or a pharmaceutically acceptable salt thereof, wherein: X, Y, and Z are each independently CR or NR′, wherein R is H or C₁₋₆ alkyl and R′ is H, C₁₋₆ alkyl, or absent; R₁ is hydrogen, cyano, halo, C₁₋₆ alkyl, C₁₋₆ alkoxy, —C(═O)NR₁₃R₁₄, —(CH₂)_{q}C(═O)NR₁₃R₁₄, CF₃, —OCF₃, or —S(═O)₂R₂₀; q is 0, 1, 2, 3, or 4; R₂₀ is hydrogen or C₁₋₆ alkyl, C₁₋₆ alkoxy, or —NR₁₃R₁₄; R₂ is hydrogen, halo, C₁₋₆ alkoxy, C₁₋₆ alkyl, or R₂ and R₃ taken together with the atoms to which they are attached form a 5-membered heterocyclyl; R₃ is hydrogen, hydroxyl, halo, C₁₋₆ alkyl, C₁₋₆ alkoxy, or R₂ and R₃ taken together with the atoms to which they are attached form a 5-membered heterocyclyl; R₄ is hydrogen, heteroaryl, heterocyclyl, —C(═O)NR₅R₆, —NR₇C(═O)R₈, —NR₉R₁₀, C₁₋₆ alkyl, C₁₋₆ alkoxy, —S(═O)₂R₂₀, or R₄ and R₁₁ taken together with the atoms to which they are attached join together to form a heteroaryl; R₁₁ is hydrogen, halo, cyano, C₁₋₆ alkyl, C₁₋₆ alkoxy, —NR₁₃R₁₄, CF₃, —OCF₃, —S(═O)₂R₂₀, R₄ and R₁₁ taken together with the atoms to which they are attached join together to form a heteroaryl, or R₁₁ and R₁₂ taken together with the atoms to which they are attached join together to form a heteroaryl; R₁₂ is hydrogen, halo, cyano, C₁₋₆ alkyl, C₁₋₆ alkoxy, —NR₁₃R₁₄, CF₃, —OCF₃, —S(═O)₂R₂₀, or R₁₁ and R₁₂ taken together with the atoms to which they are attached join together to form a heteroaryl; R₅, R₆, R₇, R₈, R₉, R₁₀, R₁₃, and R₁₄ are each independently H, C₁₋₆ alkyl, cycloalkyl, aryl, heterocyclyl, heteroaryl, alkylamino, or —NR₁₅R₁₆; and R₁₅ and R₁₆ are each independently H, C₁₋₆ alkyl, cycloalkyl, aryl, heterocyclyl, heteroaryl, or alkylamino.

In some embodiments of Formula (I), the compound has the structure:

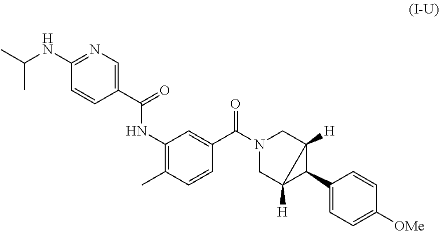 [SMILES: COc1ccc([C@H]2[C@@H]3CN(C(=O)c4ccc(C)c(NC(=O)c5ccc(NC(C)C)nc5)c4)C[C@@H]32)cc1]

or a pharmaceutically acceptable salt thereof.

In some embodiments of Formula (I), the compound has the structure:

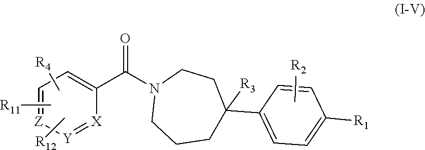

or a pharmaceutically acceptable salt thereof, wherein: X, Y, and Z are each independently CR or NR′, wherein R is H or C₁₋₆ alkyl and R′ is H, C₁₋₆ alkyl, or absent; R₁ is hydrogen, cyano, halo, C₁₋₆ alkyl, C₁₋₆ alkoxy, —C(═O)NR₁₃R₁₄, —(CH₂)_{q}C(═O)NR₁₃R₁₄, CF₃, —OCF₃, or —S(═O)₂R₂₀; q is 0, 1, 2, 3, or 4; R₂₀ is hydrogen or C₁₋₆ alkyl, C₁₋₆ alkoxy, or —NR₁₃R₁₄; R₂ is hydrogen, halo, C₁₋₆ alkoxy, C₁₋₆ alkyl, or R₂ and R₃ taken together with the atoms to which they are attached form a 5-membered heterocyclyl; R₃ is hydrogen, hydroxyl, halo, C₁₋₆ alkyl, C₁₋₆ alkoxy, or R₂ and R₃ taken together with the atoms to which they are attached form a 5-membered heterocyclyl; R₄ is hydrogen, heteroaryl, heterocyclyl, —C(═O)NR₅R₆, —NR₇C(═O)R₈, —NR₉R₁₀, C₁₋₆ alkyl, C₁₋₆ alkoxy, —S(═O)₂R₂₀, or R₄ and R₁₁ taken together with the atoms to which they are attached join together to form a heteroaryl; R₁₁ is hydrogen, halo, cyano, C₁₋₆ alkyl, C₁₋₆ alkoxy, —NR₁₃R₁₄, CF₃, —OCF₃, —S(═O)₂R₂₀, R₄ and R₁₁ taken together with the atoms to which they are attached join together to form a heteroaryl, or R₁₁ and R₁₂ taken together with the atoms to which they are attached join together to form a heteroaryl; R₁₂ is hydrogen, halo, cyano, C₁₋₆ alkyl, C₁₋₆ alkoxy, —NR₁₃R₁₄, CF₃, —OCF₃, —S(═O)₂R₂₀, or R₁₁ and R₁₂ taken together with the atoms to which they are attached join together to form a heteroaryl; R₅, R₆, R₇, R₈, R₉, R₁₀, R₁₃, and R₁₄ are each independently H, C₁₋₆ alkyl, cycloalkyl, aryl, heterocyclyl, heteroaryl, alkylamino, or —NR₁₅R₁₆; and R₁₅ and R₁₆ are each independently H, C₁₋₆ alkyl, cycloalkyl, aryl, heterocyclyl, heteroaryl, or alkylamino.

In some embodiments of Formula (I), the compound has the structure:

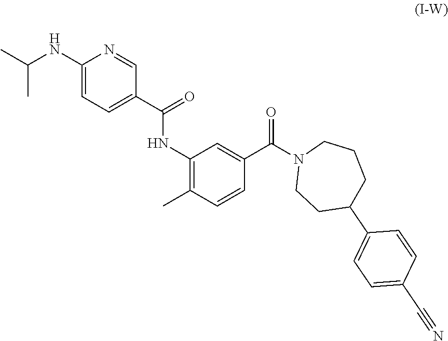 [SMILES: Cc1ccc(C(=O)N2CCCC(c3ccc(C#N)cc3)CC2)cc1NC(=O)c1ccc(NC(C)C)nc1]

or a pharmaceutically acceptable salt thereof.

In some embodiments of Formula (I), the FASN inhibitor has the structure of one of the following:

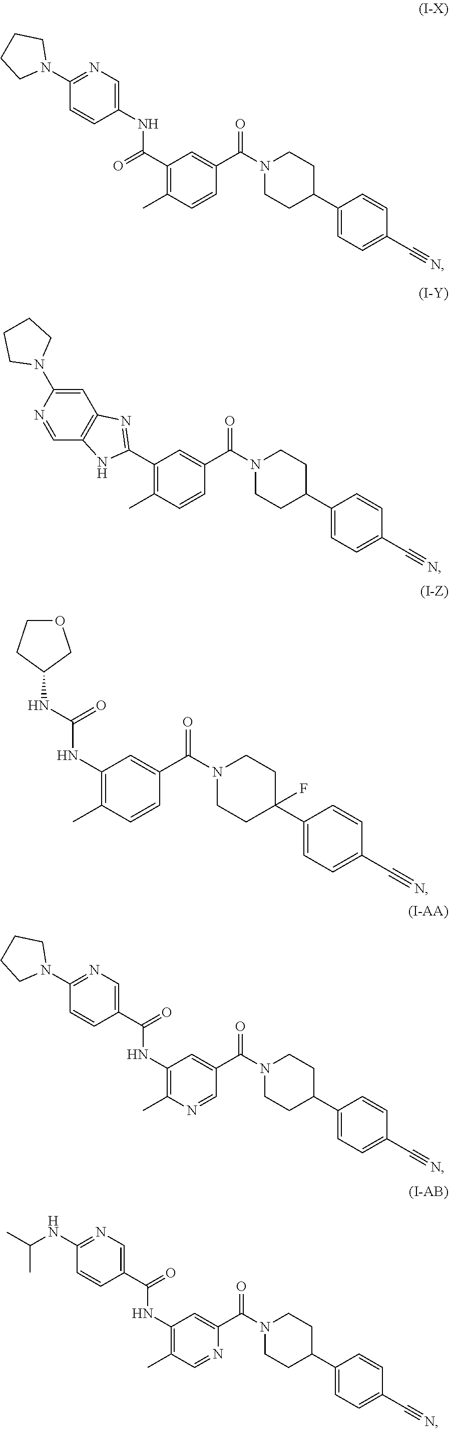 [SMILES: Cc1ccc(C(=O)N2CCC(c3ccc(C#N)cc3)CC2)cc1C(=O)Nc1ccc(N2CCCC2)nc1 ; Cc1ccc(C(=O)N2CCC(c3ccc(C#N)cc3)CC2)cc1-c1nc2cc(N3CCCC3)ncc2[nH]1 ; Cc1ccc(C(=O)N2CCC(F)(c3ccc(C#N)cc3)CC2)cc1NC(=O)N[C@@H]1CCOC1 ; Cc1ncc(C(=O)N2CCC(c3ccc(C#N)cc3)CC2)cc1NC(=O)c1ccc(N2CCCC2)nc1 ; Cc1cnc(C(=O)N2CCC(c3ccc(C#N)cc3)CC2)cc1NC(=O)c1ccc(NC(C)C)nc1]

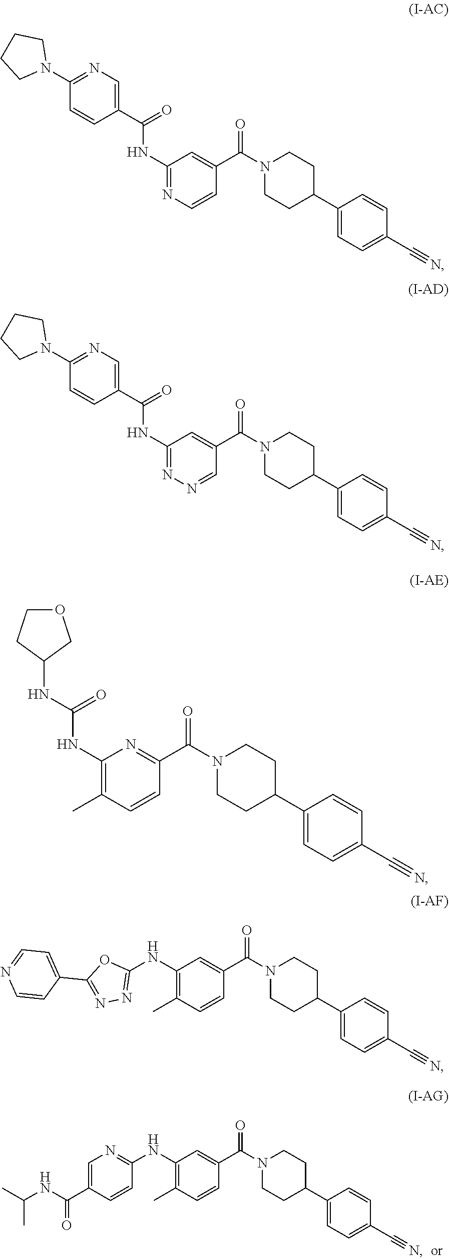 [SMILES: N#Cc1ccc(C2CCN(C(=O)c3ccnc(NC(=O)c4ccc(N5CCCC5)nc4)c3)CC2)cc1 ; N#Cc1ccc(C2CCN(C(=O)c3cnnc(NC(=O)c4ccc(N5CCCC5)nc4)c3)CC2)cc1 ; Cc1ccc(C(=O)N2CCC(c3ccc(C#N)cc3)CC2)nc1NC(=O)NC1CCOC1 ; Cc1ccc(C(=O)N2CCC(c3ccc(C#N)cc3)CC2)cc1Nc1nnc(-c2ccncc2)o1 ; Cc1ccc(C(=O)N2CCC(c3ccc(C#N)cc3)CC2)cc1Nc1ccc(C(=O)NC(C)C)cn1]

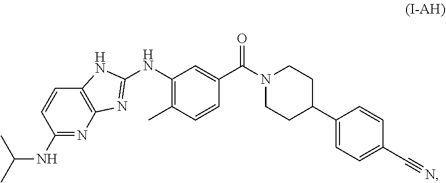 [SMILES: Cc1ccc(C(=O)N2CCC(c3ccc(C#N)cc3)CC2)cc1Nc1nc2nc(NC(C)C)ccc2[nH]1]

or a pharmaceutically acceptable salt thereof.

In some embodiments, the FASN inhibitor is a compound of Formula (II):

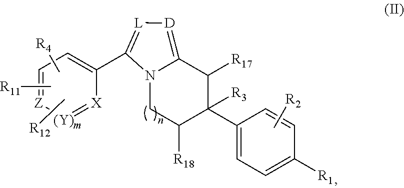

or a pharmaceutically acceptable salt thereof, wherein: X, Y, and Z are each, independently, CR or NR′, wherein R is hydrogen or C₁₋₆ alkyl and R′ is hydrogen, C₁₋₆ alkyl, or absent; L and D are each, independently, C or N; R₁ is hydrogen, cyano, halo, C₁₋₆ alkyl, C₁₋₆ alkyloxy, —C(═O)NR₁₃R₁₄, —(CH₂)_{q}C(═O)NR₁₃R₁₄, CF₃, —OCF₃, or —S(═O)₂R₂₀; q is 0, 1, 2, 3, or 4; R₂₀ is hydrogen, C₁₋₆ alkyl, C₁₋₆ alkoxy, or —NR₁₃R₁₄; R₂ is hydrogen, halo, C₁₋₆ alkoxy, C₁₋₆ alkyl, or R₂ and R₃ taken together with the atoms to which they are attached form a 5-membered heterocyclyl, R₃ is hydrogen, hydroxyl, halo, C₁₋₆ alkyl, C₁₋₆ alkoxy, or R₂ and R₃ taken together with the atoms to which they are attached form a 5-membered heterocyclyl; R₄ is hydrogen, heteroaryl, heterocyclyl, —C(═O)NR₅R₆, —NR₇C(═O)R₈, —NR₉R₁₀, C₁₋₆ alkyl, C₁₋₆ alkoxy, —S(═O)₂R₂₀, or R₄ and R₁₁ taken together with the atoms to which they are attached join together to form a heteroaryl; R₁₁ is hydrogen, halo, cyano, C₁₋₆ alkyl, C₁₋₆ alkoxy, —NR₁₃R₁₄, CF₃, —OCF₃, —S(═O)₂R₂₀, R₄ and R₁₁ taken together with the atoms to which they are attached join together to form a heteroaryl, or R₁₁ and R₁₂ taken together with the atoms to which they are attached join together to form a heteroaryl; R₁₂ is hydrogen, halo, cyano, C₁₋₆ alkyl, C₁₋₆ alkoxy, —NR₁₃R₁₄, CF₃, —OCF₃, —S(═O)₂R₂₀, or R₁₁ and R₁₂ taken together with the atoms to which they are attached join together to form a heteroaryl; R₅, R₆, R₇, R₈, R₉R₁₀, R₁₃, and R₁₄ are each independently hydrogen, C₁₋₆ alkyl, cycloalkyl, aryl, heterocyclyl, heteroaryl, hydroxyalkyl, alkylamino, —NR₁₅R₁₆, or —S(═O)₂R₂₀; R₁₅ and R₁₆ are each, independently, hydrogen, C₁₋₆ alkyl, cycloalkyl, aryl, heterocyclyl, heteroaryl, or alkylamino; R₁₇ and R₁₈ are each independently hydrogen or alkyl or can optionally join together to form a bond; n is 1 or 2; and m is 0 or 1.

In some embodiments of Formula (II), the compound has the structure:

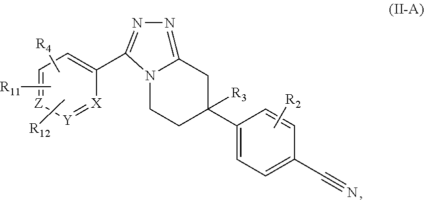

or a pharmaceutically acceptable salt thereof, wherein: X, Y, and Z are each independently CR or NR′, wherein R is H or C₁₋₆ alkyl and R′ is H, C₁₋₆ alkyl, or absent; R₂ is hydrogen, halo, C₁₋₆ alkoxy, C₁₋₆ alkyl, or R₂ and R₃ taken together with the atoms to which they are attached form a 5-membered heterocyclyl; R₃ is hydrogen, hydroxyl, halo, C₁₋₆ alkyl, C₁₋₆ alkoxy, or R₂ and R₃ taken together with the atoms to which they are attached form a 5-membered heterocyclyl; R₄ is hydrogen, heteroaryl, heterocyclyl, —C(═O)NR₅R₆, —NR₇C(═O)R₈, —NR₉R₁₀, C₁₋₆ alkyl, C₁₋₆ alkoxy, —S(═O)₂R₂₀, or R₄ and R₁₁ taken together with the atoms to which they are attached join together to form a heteroaryl; R₂₀ is hydrogen or C₁₋₆ alkyl, C₁₋₆ alkoxy, or —NR₁₃R₁₄; R₁₁ is hydrogen, halo, cyano, C₁₋₆ alkyl, C₁₋₆ alkoxy, —NR₁₃R₁₄, CF₃, —OCF₃, —S(═O)₂R₂₀, R₄ and R₁₁ taken together with the atoms to which they are attached join together to form a heteroaryl, or R₁₁ and R₁₂ taken together with the atoms to which they are attached join together to form a heteroaryl; R₁₂ is hydrogen, halo, cyano, C₁₋₆ alkyl, C₁₋₆ alkoxy, —NR₁₃R₁₄, CF₃, —OCF₃, —S(═O)₂R₂₀, or R₁₁ and R₁₂ taken together with the atoms to which they are attached join together to form a heteroaryl; R₅, R₆, R₇, R₈, R₉, R₁₀, R₁₃, and R₁₄ are each independently H, C₁₋₆ alkyl, cycloalkyl, aryl, heterocyclyl, heteroaryl, alkylamino, or —NR₁₅R₁₆; and R₁₅ and R₁₆ are each independently H, C₁₋₆ alkyl, cycloalkyl, aryl, heterocyclyl, heteroaryl, or alkylamino.

In some embodiments, the compound has the structure:

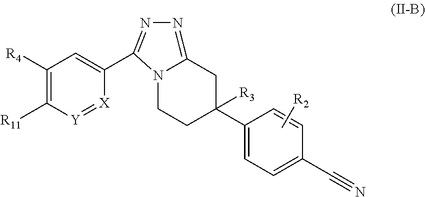

or a pharmaceutically acceptable salt thereof, wherein: X and Y are each independently CR or NR′, wherein R is H or C₁₋₆ alkyl and R′ is H, C₁₋₆ alkyl, or absent; R₂ is hydrogen, halo, C₁₋₆ alkoxy, C₁₋₆ alkyl, or R₂ and R₃ taken together with the atoms to which they are attached form a 5-membered heterocyclyl; R₃ is hydrogen, hydroxyl, halo, C₁₋₆ alkyl, C₁₋₆ alkoxy, or R₂ and R₃ taken together with the atoms to which they are attached form a 5-membered heterocyclyl; R₄ is hydrogen, heteroaryl, heterocyclyl, —C(═O)NR₅R₆, —NR₇C(═O)R₈, —NR₉R₁₀, C₁₋₆ alkyl, C₁₋₆ alkoxy, —S(═O)₂R₂₀, or R₄ and R₁₁ taken together with the atoms to which they are attached join together to form a heteroaryl; R₂₀ is hydrogen or C₁₋₆ alkyl, C₁₋₆ alkoxy, or —NR₁₃R₁₄; R₁₁ is hydrogen, halo, cyano, C₁₋₆ alkyl, C₁₋₆ alkoxy, —NR₁₃R₁₄, CF₃, —OCF₃, —S(═O)₂R₂₀, or R₄ and R₁₁ taken together with the atoms to which they are attached join together to form a heteroaryl;

R₅, R₆, R₇, R₈, R₉, and R₁₀ are each independently H, C₁₋₆ alkyl, cycloalkyl, aryl, heterocyclyl, heteroaryl, alkylamino, or —NR₁₅R₁₆; and R₁₅ and R₁₆ are each independently H, C₁₋₆ alkyl, cycloalkyl, aryl, heterocyclyl, heteroaryl, or alkylamino.

In some embodiments, the FASN inhibitor is a compound of one of the following:

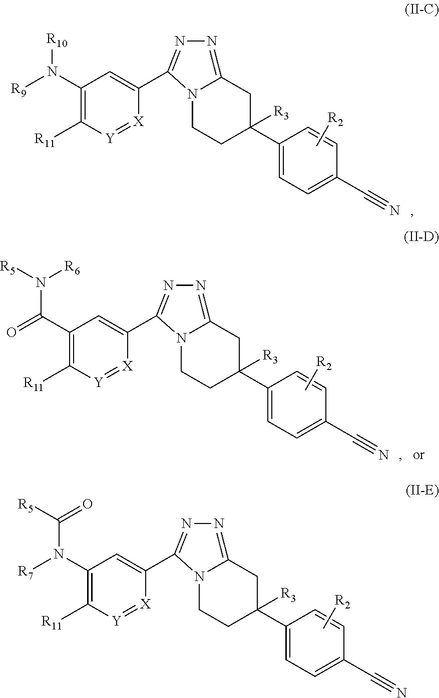

or a pharmaceutically acceptable salt thereof, wherein: X and Y are each independently CR or NR′, wherein R is H or C₁₋₆ alkyl and R′ is H, C₁₋₆ alkyl, or absent; R₂ is hydrogen, halo, C₁₋₆ alkoxy, C₁₋₆ alkyl, or R₂ and R₃ taken together with the atoms to which they are attached form a 5-membered heterocyclyl; R₃ is hydrogen, hydroxyl, halo, C₁₋₆ alkyl, C₁₋₆ alkoxy, or R₂ and R₃ taken together with the atoms to which they are attached form a 5-membered heterocyclyl; R₁₁ is hydrogen, halo, cyano, C₁₋₆ alkyl, C₁₋₆ alkoxy, —NR₁₃R₁₄, CF₃, —OCF₃, or —S(═O)₂R₂₀; R₂₀ is hydrogen or C₁₋₆ alkyl, C₁₋₆ alkoxy, or —NR₁₃R₁₄; R₅, R₆, R₇, R₈, R₉, and R₁₀ are each independently H, C₁₋₆ alkyl, cycloalkyl, aryl, heterocyclyl, heteroaryl, alkylamino, or —NR₁₅R₁₆; and R₁₅ and R₁₆ are each independently H, C₁₋₆ alkyl, cycloalkyl, aryl, heterocyclyl, heteroaryl, or alkylamino.

In some embodiments, the compound has the following structure:

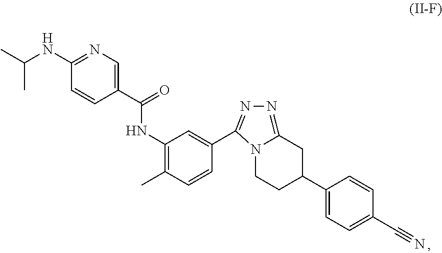 [SMILES: Cc1ccc(-c2nnc3n2CCC(c2ccc(C#N)cc2)C3)cc1NC(=O)c1ccc(NC(C)C)nc1]

or a pharmaceutically acceptable salt thereof.

In some embodiments, the FASN inhibitor is a compound of Formula (III):

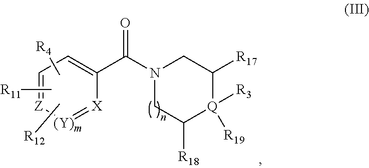

or a pharmaceutically acceptable salt thereof, wherein: X, Y, and Z are each independently CR or NR′, wherein R is hydrogen or C₁₋₆ alkyl and R′ is hydrogen, C₁₋₆ alkyl, or absent; Q is C or N; R₃ is hydrogen, hydroxyl, halo, C₁₋₆ alkyl, C₁₋₆ alkoxy, or if Q is N then R₃ is absent; R₄ is hydrogen, heteroaryl, heterocyclyl, —C(═O)NR₅R₆, —NR₇C(═O)R₈, —NR₉R₁₀, C₁₋₆ alkyl, C₁₋₆ alkoxy, —S(═O)₂R₂₀, or R₄ and R₁₁ taken together with the atoms to which they are attached join together to form a heteroaryl; R₁₁ is hydrogen, halo, cyano, C₁₋₆ alkyl, C₁₋₆ alkoxy, —NR₁₃R₁₄, CF₃, —OCF₃, —S(═O)₂R₂₀, R₄ and R₁₁ taken together with the atoms to which they are attached join together to form a heteroaryl, or R₁₁ and R₁₂ taken together with the atoms to which they are attached join together to form a heteroaryl; R₁₂ is hydrogen, halo, cyano, C₁₋₆ alkyl, C₁₋₆ alkoxy, —NR₁₃R₁₄, CF₃, —OCF₃, —S(═O)₂R₂₀, or R₁₁ and R₁₂ taken together with the atoms to which they are attached join together to form a heteroaryl; R₂₀ is hydrogen or C₁₋₆ alkyl, C₁₋₆ alkoxy, or —NR₁₃R₁₄; R₅, R₆, R₇, R₈, R₉R₁₀, R₁₃, and R₁₄ are each independently hydrogen, C₁₋₆ alkyl, cycloalkyl, aryl, heterocyclyl, heteroaryl, hydroxyalkyl, alkylamino, —NR₁₅R₁₆, or —S(═O)₂R₂₀; R₁₅ and R₁₆ are each, independently, hydrogen, C₁₋₆ alkyl, cycloalkyl, aryl, heterocyclyl, heteroaryl, or alkylamino; R₁₇ and R₁₈ are each, independently, hydrogen or alkyl or can optionally join together to form a bond; R₁₉ is aryl, heteroaryl, cycloalkyl, or heterocyclyl; n is 0, 1, or 2; and m is 0 or 1.

In some embodiments, the FASN inhibitor has the structure of one of the following:

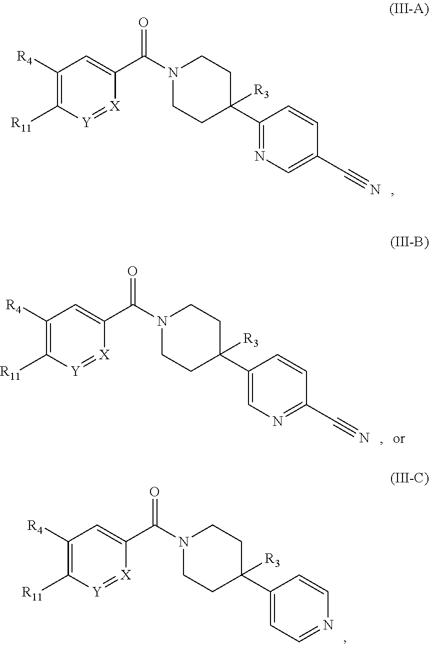

or a pharmaceutically acceptable salt thereof, wherein: X and Y are each independently CR or NR′, wherein R is H or C₁₋₆ alkyl and R′ is H, C₁₋₆ alkyl, or absent; R₃ is hydrogen, hydroxyl, halo, C₁₋₆ alkyl, or C₁₋₆ alkoxy; R₄ is hydrogen, heteroaryl, heterocyclyl, —C(═O)NR₅R₆, —NR₇C(═O)R₈, —NR₉R₁₀, C₁₋₆ alkyl, C₁₋₆ alkoxy, —S(═O)₂R₂₀, or R₄ and R₁₁ taken together with the atoms to which they are attached join together to form a heteroaryl; R₁₁ is hydrogen, halo, cyano, C₁₋₆ alkyl, C₁₋₆ alkoxy, —NR₁₃R₁₄, CF₃, —OCF₃, —S(═O)₂R₂₀, or R₄ and R₁₁ taken together with the atoms to which they are attached join together to form a heteroaryl; R₅, R₆, R₇, R₈, R₉, and R₁₀ are each independently H, C₁₋₆ alkyl, cycloalkyl, aryl, heterocyclyl, heteroaryl, alkylamino, or —NR₁₅R₁₆; and R₁₅ and R₁₆ are each independently H, C₁₋₆ alkyl, cycloalkyl, aryl, heterocyclyl, heteroaryl, or alkylamino.

In some embodiments, the FASN inhibitor has the structure of one of the following:

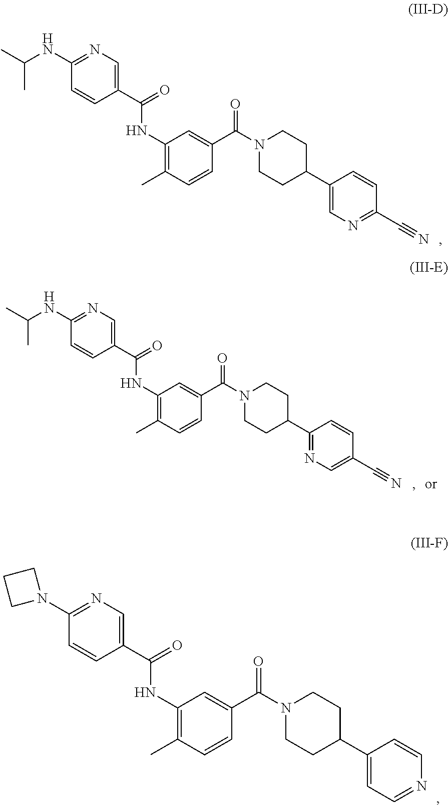 [SMILES: Cc1ccc(C(=O)N2CCC(c3ccc(C#N)nc3)CC2)cc1NC(=O)c1ccc(NC(C)C)nc1 ; Cc1ccc(C(=O)N2CCC(c3ccc(C#N)cn3)CC2)cc1NC(=O)c1ccc(NC(C)C)nc1 ; Cc1ccc(C(=O)N2CCC(c3ccncc3)CC2)cc1NC(=O)c1ccc(N2CCC2)nc1]

or a pharmaceutically acceptable salt thereof.

In some embodiments, the FASN inhibitor is a compound of Formula (IV-A), (IV-B), or (IV-C):

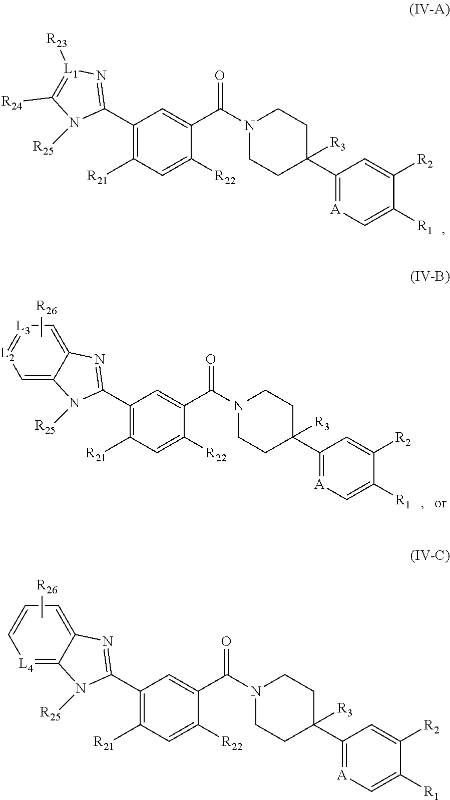

or a pharmaceutically acceptable salt thereof, wherein: L₁, L₂, L₃, L₄, and A are each, independently, CH or N; R₁ is hydrogen, cyano, halo, C₁₋₆ alkyl, C₁₋₆ alkoxy, —C(═O)NR₁₃R₁₄, —(CH₂)_{q}C(═O)NR₁₃R₁₄, CF₃, —OCF₃, or —S(═O)₂R₂₀; q is 0, 1, 2, 3, or 4; R₂₀ is hydrogen or C₁₋₆ alkyl, C₁₋₆ alkoxy, or —NR₁₃R₁₄; R₂ is hydrogen, halo, C₁₋₆ alkoxy, or C₁₋₆ alkyl; R₃ is hydrogen, hydroxyl, halo, C₁₋₆ alkyl, or C₁₋₆ alkoxy; R₂₁ and R₂₂ are each independently hydrogen, halo, cyano, C₁₋₆ alkyl, C₁₋₆ alkoxy, CF₃, —OCF₃, or S(═O)₂R₂₀; R₂₃ is hydrogen, —NR₁₃R₁₄, C₁₋₆ alkyl, C₁₋₆ alkoxy, is absent if L₁ is N, or R₂₃ and R₂₄ taken together with the atoms to which they are attached join together to form a heterocyclyl, heteroaryl, or cycloalkyl; R₂₄ is hydrogen, —NR₁₃R₁₄, C₁₋₆ alkyl, C₁₋₆ alkoxy, —(C₁₋₆ alkoxy)(heterocyclyl), heterocyclyl, or R₂₃ and R₂₄ taken together with the atoms to which they are attached join together to form a heterocyclyl, heteroaryl, or cycloalkyl; R₂₆ is hydrogen, heteroaryl, heterocycyl, —NR₁₃R₁₄, or —S(═O)₂R₂₀; R₁₃ and R₁₄ are each independently hydrogen, C₁₋₆ alkyl, cycloalkyl, aryl, heterocyclyl, heteroaryl, hydroxyalkyl, alkylamino, —NR₁₅R₁₆, or —S(═O)₂R₂₀; R₂₅ is hydrogen, C₁₋₆ alkyl, or C₁₋₆ alkoxy; and R₁₅ and R₁₆ are each independently hydrogen, C₁₋₆ alkyl, C₁₋₆ alkoxy, cycloalkyl, aryl, heterocyclyl, heteroaryl, hydroxyalkyl, or alkylamino.

In some embodiments, the FASN inhibitor is a compound of Formula (IV-D) or (IV-E):

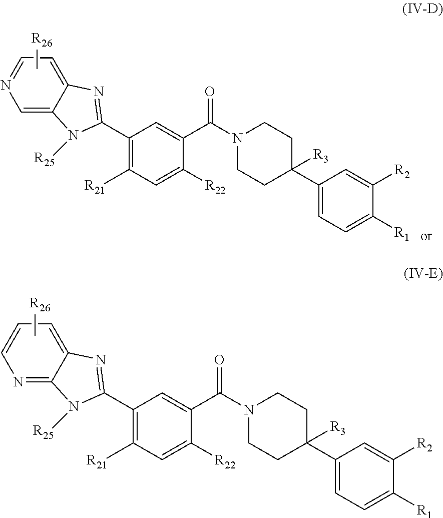

or a pharmaceutically acceptable salt thereof, wherein: R₁ is hydrogen, cyano, halo, C₁₋₆ alkyl, C₁₋₆ alkoxy, —C(═O)NR₁₃R₁₄, —(CH₂)_{q}C(═O)NR₁₃R₁₄, CF₃, —OCF₃, or —S(═O)₂R₂₀; q is 0, 1, 2, 3, or 4; R₂₀ is hydrogen or C₁₋₆ alkyl, C₁₋₆ alkoxy, or —NR₁₃R₁₄; R₂ is hydrogen, halo, C₁₋₆ alkoxy, or C₁₋₆ alkyl; R₃ is hydrogen, hydroxyl, halo, C₁₋₆ alkyl, or C₁₋₆ alkoxy; R₂₁ and R₂₂ are each independently hydrogen, halo, cyano, C₁₋₆ alkyl, C₁₋₆ alkoxy, CF₃, —OCF₃, or —S(═O)₂R₂₀; R₂₆ is hydrogen, heteroaryl, heterocycyl, —NR₁₃R₁₄, or —S(═O)₂R₂₀; R₁₃ and R₁₄ are each independently hydrogen, C₁₋₆ alkyl, cycloalkyl, aryl, heterocyclyl, heteroaryl, hydroxyalkyl, alkylamino, —NR₁₅R₁₆, or —S(═O)₂R₂₀; R₂₅ is hydrogen, C₁₋₆ alkyl, or C₁₋₆ alkoxy; and R₁₅ and R₁₆ are each independently hydrogen, C₁₋₆ alkyl, C₁₋₆ alkoxy, cycloalkyl, aryl, heterocyclyl, heteroaryl, hydroxyalkyl, or alkylamino.

In some embodiments, the FASN inhibitor is a compound of Formula (IV-F) or (IV-G):

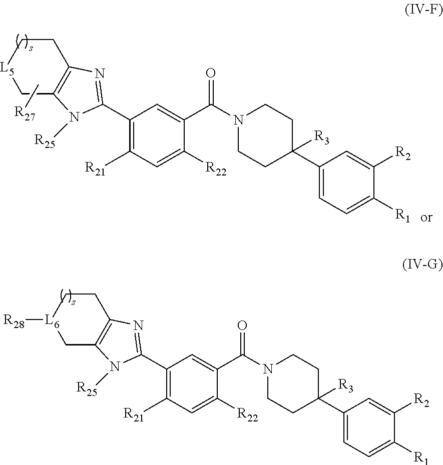

or a pharmaceutically acceptable salt thereof, wherein: R₁ is hydrogen, cyano, halo, C₁₋₆ alkyl, C₁₋₆ alkoxy, —C(═O)NR₁₃R₁₄, —(CH₂)_{q}C(═O)NR₁₃R₁₄, CF₃, —OCF₃, or —S(═O)₂R₂₀; q is 0, 1, 2, 3, or 4; R₂₀ is hydrogen or C₁₋₆ alkyl, C₁₋₆ alkoxy, or —NR₁₃R₁₄; R₂ is hydrogen, halo, C₁₋₆ alkoxy, or C₁₋₆ alkyl; R₃ is hydrogen, hydroxyl, halo, C₁₋₆ alkyl, or C₁₋₆ alkoxy; R₂₁ and R₂₂ are each independently hydrogen, halo, cyano, C₁₋₆ alkyl, C₁₋₆ alkoxy, CF₃, —OCF₃, or —S(═O)₂R₂₀; R₁₃ and R₁₄ are each independently hydrogen, C₁₋₆ alkyl, cycloalkyl, aryl, heterocyclyl, heteroaryl, hydroxyalkyl, alkylamino, —NR₁₅R₁₆, or —S(═O)₂R₂₀; R₂₅ is hydrogen, C₁₋₆ alkyl, or C₁₋₆ alkoxy; R₁₅ and R₁₆ are each independently hydrogen, C₁₋₆ alkyl, C₁₋₆ alkoxy, cycloalkyl, aryl, heterocyclyl, heteroaryl, hydroxyalkyl, or alkylamino; s is 0, 1, or 2; L₅ is CH₂, NH, S, or O; L₆ is CH or N; R₂₇ is hydrogen, —C(═O)R′, —S(═O)₂R₂₀; R₂₈ is hydrogen, —C(═O)R′, —S(═O)₂R₂₀, or is absent if L₆ is 0; and R′ is hydrogen, C₁₋₆ alkyl, C₁₋₆ alkoxy, —C(═O)NR₁₃R₁₄, or —NR₁₃R₁₄.

In some embodiments, R₁ is hydrogen, cyano, C₁₋₆ alkyl, C₁₋₆ alkoxy, or —C(═O)NR₁₃R₁₄. In some embodiments, R₁ is cyano. In some embodiments, R₂ is hydrogen or halo; R₂ is hydrogen. In some embodiments, R₃ is hydrogen. In some embodiments, R₂₁ and R₂₂ are each independently hydrogen or C₁₋₆ alkyl. In some embodiments, R₂₁ and R₂₂ are each independently C₁₋₆ alkyl. In some embodiments, R₂₅ is hydrogen. In some embodiments, L₂ is N. In some embodiments, L₁ is CH. In some embodiments, L₃ is CH. In some embodiments, L₄ is CH. In some embodiments, A is N. In some embodiments, A is CH. In some embodiments, R₂₆ is heterocyclyl. In some embodiments, R₂₄ is —NR₁₃R₁₄. In some embodiments, L₅ and L₆ are each independently N. In some embodiments, s is 1. In some embodiments, s is 0.

In some embodiments, the FASN inhibitor has the structure of one of the following:

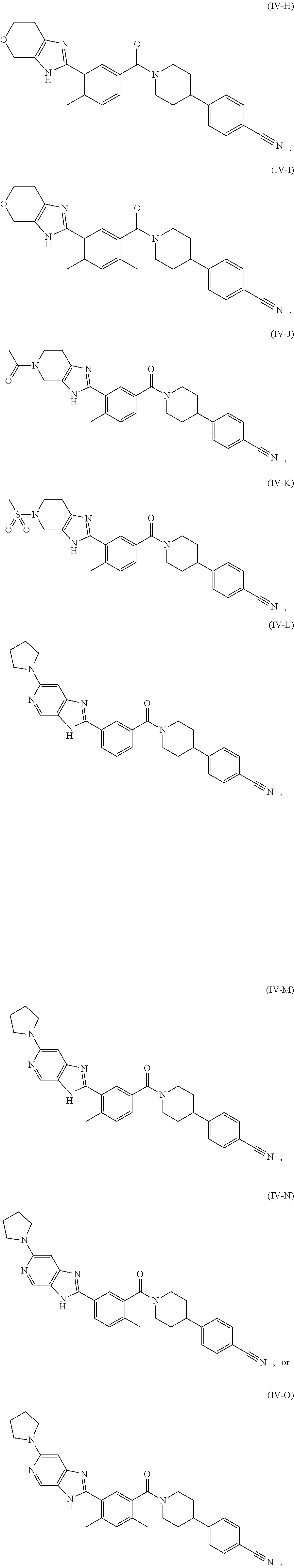

or a pharmaceutically acceptable salt thereof.

In some embodiments, the FASN inhibitor is a compound of Formula (V):

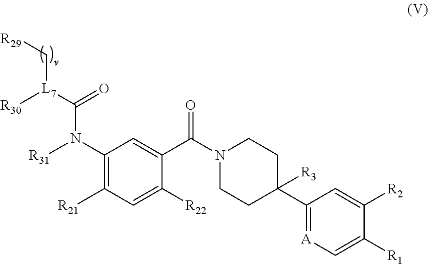

or a pharmaceutically acceptable salt thereof, wherein: L₇ is N or O, wherein R₃₀ is absent if L₇ is O; A is CH or N; R₁ is hydrogen, cyano, halo, C₁₋₆ alkyl, C₁₋₆ alkoxy, —C(═O)NR₁₃R₁₄, —(CH₂)_{q}C(═O)NR₁₃R₁₄, CF₃, —OCF₃, or —S(═O)₂R₂₀; q is, 1, 2, 3, or 4; R is 0, 1, 2, 3, or 4; R₂₀ is hydrogen or C₁₋₆ alkyl, C₁₋₆ alkoxy, or —NR₁₃R₁₄; R₂ is hydrogen, halo, C₁₋₆ alkoxy, or C₁₋₆ alkyl; R₃ is halo, C₁₋₆ alkyl, or C₁₋₆ alkoxy; R₂₁ and R₂₂ are each, independently, hydrogen, halo, cyano, C₁₋₆ alkyl, C₁₋₆ alkoxy, CF₃, —OCF₃, or —S(═O)₂R₂₀; R₂₉ and R₃₀ are each, independently, hydrogen, C₁₋₆ alkyl, C₁₋₆ alkoxy, hydroxyalkyl, heteroaryl, heterocyclyl, —NR₁₅R₁₆, —C(═O)R₄₆, —R₄₈C(═O)R₄₇, or R₂₉ and R₃₀ taken together with the atoms to which they are attached join together to form a heteroaryl or heterocyclyl, wherein R₃₀ is absent if L₇ is O; R₄₆ and R₄₇ are each independently hydrogen, C₁₋₆ alkyl, cycloalkyl, aryl, heterocyclyl, heteroaryl, hydroxyalkyl, —NR₁₅R₁₆, or —S(═O)₂R₂₀; R₄₈ is alkyl or is absent; R₃₁ is hydrogen, C₁₋₆ alkyl, or C₁₋₆ alkoxy; R₁₃ and R₁₄ are each, independently, hydrogen, C₁₋₆ alkyl, cycloalkyl, aryl, heterocyclyl, heteroaryl, hydroxyalkyl, alkylamino, —NR₁₅R₁₆, or —S(═O)₂R₂₀; R₁₅ and R₁₆ are each, independently, hydrogen, C₁₋₆ alkyl, cycloalkyl, aryl, heterocyclyl, heteroaryl, hydroxyalkyl, or alkylamino; and v is 0 or 1.

In some embodiments, the FASN inhibitor has the structure of one of the following:

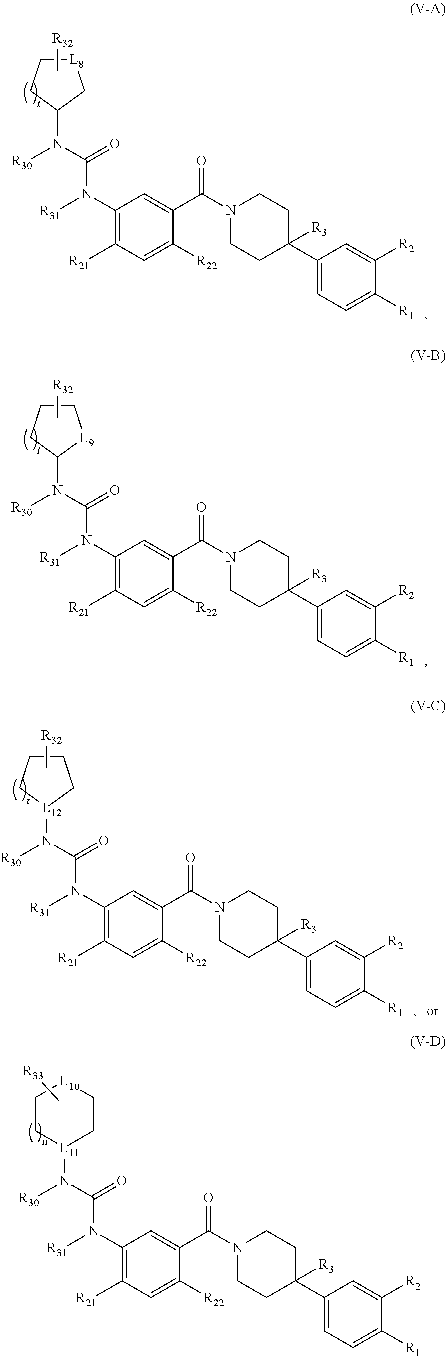

or a pharmaceutically acceptable salt thereof, wherein: R₁ is hydrogen, cyano, halo, C₁₋₆ alkyl, C₁₋₆ alkoxy, —C(═O)NR₁₃R₁₄, —(CH₂)_{q}C(═O)NR₁₃R₁₄, CF₃, —OCF₃, or —S(═O)₂R₂₀; q is 0, 1, 2, 3, or 4; R₂₀ is hydrogen or C₁₋₆ alkyl, C₁₋₆ alkoxy, or —NR₁₃R₁₄; R₂ is hydrogen, halo, C₁₋₆ alkoxy, or C₁₋₆ alkyl; R₃ is halo, C₁₋₆ alkyl, or C₁₋₆ alkoxy; R₂₁ and R₂₂ are each independently hydrogen, halo, cyano, C₁₋₆ alkyl, C₁₋₆ alkoxy, CF₃, —OCF₃, or —S(═O)₂R₂₀; R₃₀ is hydrogen, C₁₋₆ alkyl, C₁₋₆ alkoxy, hydroxyalkyl, heteroaryl, heterocyclyl, —NR₁₅R₁₆, —C(═O)R₄₆, or —R₄₈C(═O)R₄₇, wherein R₃₀ is absent if L₇ is O; R₄₆ and R₄₇ are each independently hydrogen, C₁₋₆ alkyl, cycloalkyl, aryl, heterocyclyl, heteroaryl, hydroxyalkyl, —NR₁₅R₁₆, or —S(═O)₂R₂₀; R₄₈ is alkyl or is absent; R₃₁ is hydrogen, C₁₋₆ alkyl, or C₁₋₆ alkoxy; R₁₃ and R₁₄ are each independently hydrogen, C₁₋₆ alkyl, cycloalkyl, aryl, heterocyclyl, heteroaryl, hydroxyalkyl, alkylamino, —NR₁₅R₁₆, or —S(═O)₂R₂₀; R₁₅ and R₁₆ are each independently hydrogen, C₁₋₆ alkyl, cycloalkyl, aryl, heterocyclyl, heteroaryl, hydroxyalkyl, or alkylamino; L₈, L₉, and L₁₀ are each independently CH₂, NH, or O; L₁₁ and L₁₂ are each independently CH or N; R₃₂ and R₃₃ are each independently hydrogen, C₁₋₆ alkyl, C₁₋₆ alkoxy, —S(═O)₂R₂₀, —C(═O)R₄₆, hydroxyalkyl, hydroxyl, or are absent; u is 0, 1, or 2; and t is 0, 1, or 2.

In some embodiments, L₇ is N. In some embodiments, L₇ is O. In some embodiments, A is N. In some embodiments, A is CH. In some embodiments, R₁ is hydrogen, cyano, C₁₋₆ alkyl, C₁₋₆ alkoxy, or —C(═O)NR₁₃R₁₄. In some embodiments, R₁ is cyano. In some embodiments, R₂ is hydrogen or halo. In some embodiments, R₂ is hydrogen. In some embodiments, R₃ is fluorine. In some embodiments, R₂₁ and R₂₂ are each independently hydrogen or C₁₋₆ alkyl. In some embodiments, R₂₁ and R₂₂ are each independently C₁₋₆ alkyl. In some embodiments, R₃₁ is hydrogen. In some embodiments, R₃₀ is hydrogen. In some embodiments, L₈ is O. In some embodiments, L₉ is O. In some embodiments, L₁₀ is O and L₁₁ is N. In some embodiments, L₁₂ is N. In some embodiments, R₃₂ and R₃₃ are each independently hydrogen.

In some embodiments, the FASN inhibitor has the structure of one of the following:

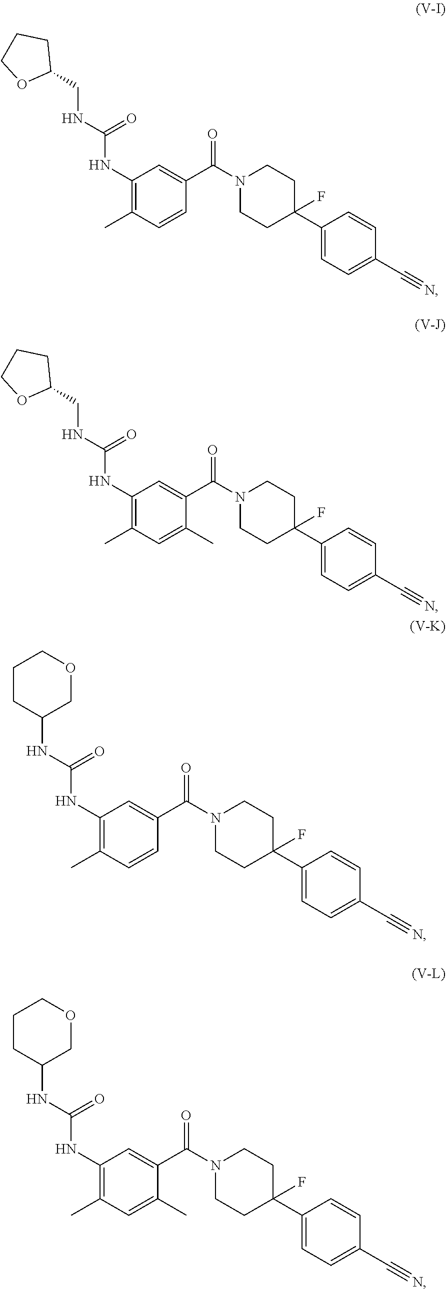 [SMILES: Cc1ccc(C(=O)N2CCC(F)(c3ccc(C#N)cc3)CC2)cc1NC(=O)NC[C@H]1CCCO1 ; Cc1cc(C)c(C(=O)N2CCC(F)(c3ccc(C#N)cc3)CC2)cc1NC(=O)NC[C@H]1CCCO1 ; Cc1ccc(C(=O)N2CCC(F)(c3ccc(C#N)cc3)CC2)cc1NC(=O)NC1CCCOC1 ; Cc1cc(C)c(C(=O)N2CCC(F)(c3ccc(C#N)cc3)CC2)cc1NC(=O)NC1CCCOC1]

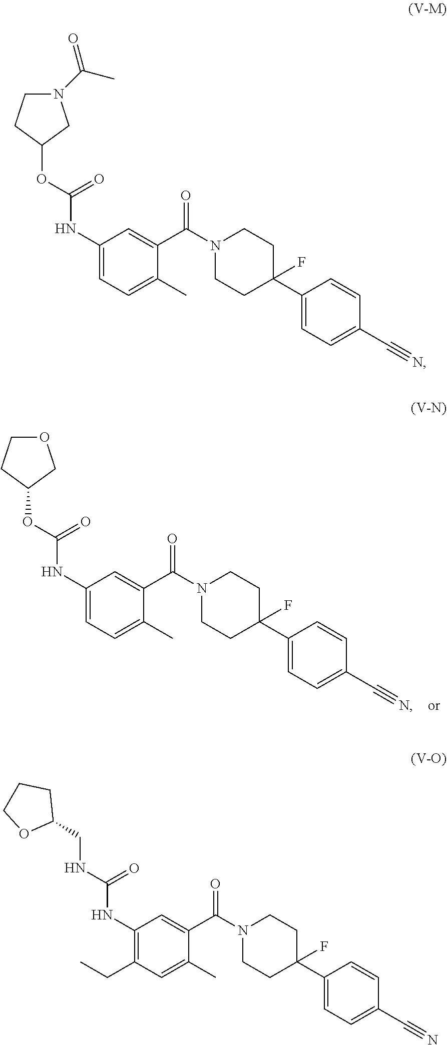 [SMILES: CC(=O)N1CCC(OC(=O)Nc2ccc(C)c(C(=O)N3CCC(F)(c4ccc(C#N)cc4)CC3)c2)C1 ; Cc1ccc(NC(=O)O[C@@H]2CCOC2)cc1C(=O)N1CCC(F)(c2ccc(C#N)cc2)CC1 ; CCc1cc(C)c(C(=O)N2CCC(F)(c3ccc(C#N)cc3)CC2)cc1NC(=O)NC[C@H]1CCCO1]

or a pharmaceutically acceptable salt thereof.

In some embodiments, the FASN inhibitor is a compound of Formula (VI-A) or (VI-B):

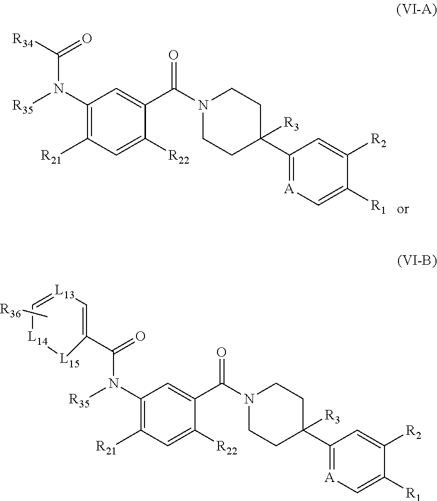

or a pharmaceutically acceptable salt thereof, wherein: L₁₃, L₁₄, L₁₅, and A are each, independently, CH or N; R₁ is hydrogen, cyano, halo, C₁₋₆ alkyl, C₁₋₆ alkoxy, —C(═O)NR₁₃R₁₄, —(CH₂)_{q}C(═O)NR₁₃R₁₄, CF₃, —OCF₃, or —S(═O)₂R₂₀; q is 0, 1, 2, 3, or 4; R₂₀ is hydrogen, C₁₋₆ alkyl, C₁₋₆ alkoxy, or —NR₁₃R₁₄; R₂ is hydrogen, halo, C₁₋₆ alkoxy, or C₁₋₆ alkyl; R₃ is halo, C₁₋₆ alkyl, or C₁₋₆ alkoxy; R₂₁ and R₂₂ are each independently hydrogen, halo, cyano, C₁₋₆ alkyl, C₁₋₆ alkoxy, CF₃, —OCF₃, or —S(═O)₂R₂₀; R₃₄ is hydrogen, C₁₋₆ alkyl, C₁₋₆ alkoxy, cycloalkyl, hydroxyl, hydroxyalkyl, aryl, heterocyclyl, heteroaryl, alkylamino, CF₃, OCF₃, —S(═O)₂R₂₀, or —NR₁₅R₁₆; R₃₅ is hydrogen, C₁₋₆ alkyl, or C₁₋₆ alkoxy; R₃₆ is hydrogen, C₁₋₆ alkyl, C₁₋₆ alkoxy, —NR₁₅R₁₆, heterocyclyl, or heteroaryl; R₁₃ and R₁₄ are each independently hydrogen, C₁₋₆ alkyl, cycloalkyl, aryl, heterocyclyl, heteroaryl, hydroxyalkyl, alkylamino, —NR₁₅R₁₆, or —S(═O)₂R₂₀; and R₁₅ and R₁₆ are each independently hydrogen, C₁₋₆ alkyl, C₁₋₆ alkoxy, cycloalkyl, aryl, heterocyclyl, heteroaryl, hydroxyalkyl, or alkylamino.

In some embodiments, the FASN inhibitor has the structure of one of the following:

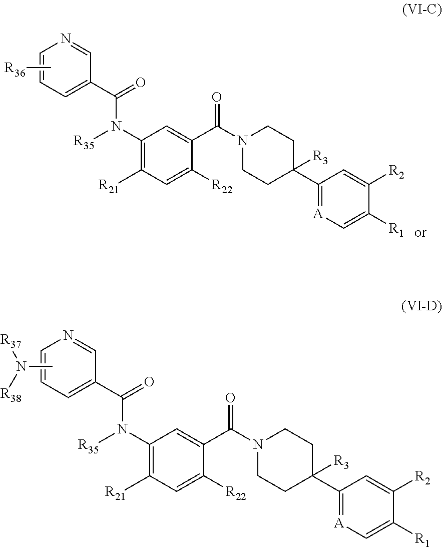

or a pharmaceutically acceptable salt thereof, wherein: R₁ is hydrogen, cyano, halo, C₁₋₆ alkyl, C₁₋₆ alkoxy, —C(═O)NR₁₃R₁₄, —(CH₂)_{q}C(═O)NR₁₃R₁₄, CF₃, —OCF₃, or —S(═O)₂R₂₀; q is 0, 1, 2, 3, or 4; R₂₀ is hydrogen or C₁₋₆ alkyl, C₁₋₆ alkoxy, or —NR₁₃R₁₄; R₂ is hydrogen, halo, C₁₋₆ alkoxy, or C₁₋₆ alkyl; R₃ is halo, C₁₋₆ alkyl, or C₁₋₆ alkoxy; R₂₁ and R₂₂ are each independently hydrogen, halo, cyano, C₁₋₆ alkyl, C₁₋₆ alkoxy, CF₃, —OCF₃, or —S(═O)₂R₂₀; R₃₅ is hydrogen, C₁₋₆ alkyl, or C₁₋₆ alkoxy; R₃₆ is hydrogen, C₁₋₆ alkyl, C₁₋₆ alkoxy, —NR₁₅R₁₆, heterocyclyl, or heteroaryl; R₁₃ and R₁₄ are each independently hydrogen, C₁₋₆ alkyl, cycloalkyl, aryl, heterocyclyl, heteroaryl, hydroxyalkyl, alkylamino, —NR₁₅R₁₆, or —S(═O)₂R₂₀; R₁₅ and R₁₆ are each independently hydrogen, C₁₋₆ alkyl, C₁₋₆ alkoxy, cycloalkyl, aryl, heterocyclyl, heteroaryl, hydroxyalkyl, or alkylamino; and R₃₇ and R₃₈ are each independently hydrogen, C₁₋₆ alkyl, C₁₋₆ alkoxy, hydroxyalkyl, heteroaryl, heterocyclyl, or R₃₇ and R₃₈ taken together with the atoms to which they are attached join together to form a heteroaryl or heterocyclyl.

In some embodiments, R₁ is hydrogen, cyano, C₁₋₆ alkyl, C₁₋₆ alkoxy, or —C(═O)NR₁₃R₁₄. In some embodiments, R₁ is cyano. In some embodiments, R₂ is hydrogen or halo. In some embodiments, R₂ is hydrogen. In some embodiments, R₃ is fluorine. In some embodiments, R₂₁ and R₂₂ are each independently hydrogen or C₁₋₆ alkyl. In some embodiments, R₂₁ and R₂₂ are each independently C₁₋₆ alkyl. In some embodiments, R₃₅ is hydrogen. In some embodiments, R₃₄ is heteroaryl; In some embodiments, R₃₄ is thienyl, pyrryl, furyl, pyridyl, pyrimidyl, pyrazinyl, pyrazolyl, oxazolyl, isoxazolyl, imidazolyl, thiazolyl, pyranyl, tetrazolyl, pyrrolyl, pyrrolinyl, pyridazinyl, triazolyl, indolyl, isoindolyl, indolizinyl, benzimidazolyl, quinolyl, isoquinolyl, indazolyl, benzotriazolyl, tetrazolopyridazinyl, oxadiazolyl, benzoxazolyl, benzoxadiazolyl, thiadiazolyl, benzothiazolyl, or benzothiadiazolyl. In some embodiments, L₁₃ is N. In some embodiments, L₁₄ and L₁₅ are each independently CH. In some embodiments, A is N. In some embodiments, A is CH.

In some embodiments, the FASN inhibitor has the structure of one of the following:

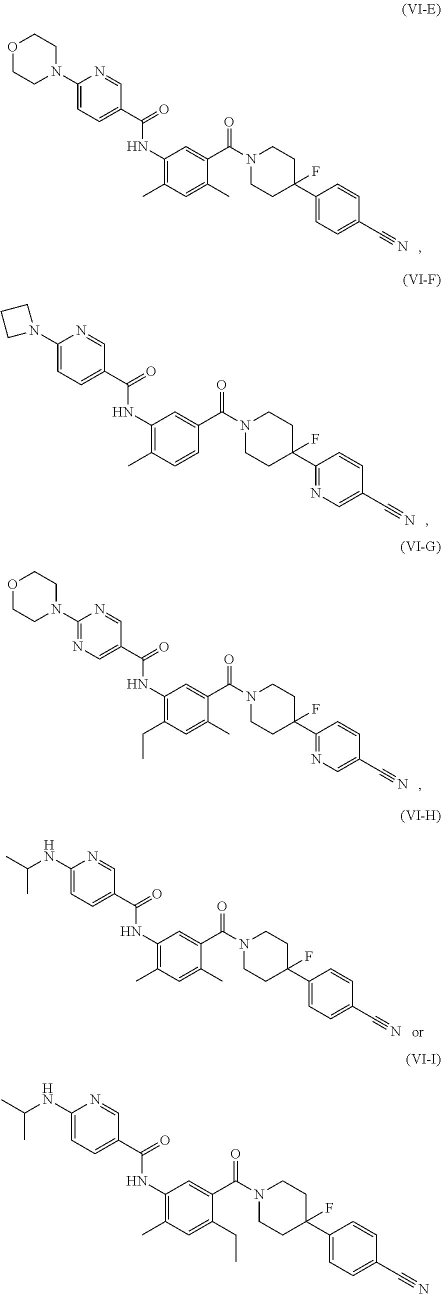 [SMILES: Cc1cc(C)c(C(=O)N2CCC(F)(c3ccc(C#N)cc3)CC2)cc1NC(=O)c1ccc(N2CCOCC2)nc1 ; Cc1ccc(C(=O)N2CCC(F)(c3ccc(C#N)cn3)CC2)cc1NC(=O)c1ccc(N2CCC2)nc1 ; CCc1cc(C)c(C(=O)N2CCC(F)(c3ccc(C#N)cn3)CC2)cc1NC(=O)c1cnc(N2CCOCC2)nc1 ; Cc1cc(C)c(C(=O)N2CCC(F)(c3ccc(C#N)cc3)CC2)cc1NC(=O)c1ccc(NC(C)C)nc1 ; CCc1cc(C)c(NC(=O)c2ccc(NC(C)C)nc2)cc1C(=O)N1CCC(F)(c2ccc(C#N)cc2)CC1]

or a pharmaceutically acceptable salt thereof.

In some embodiments, the compound has structure of Formula (VI-J):

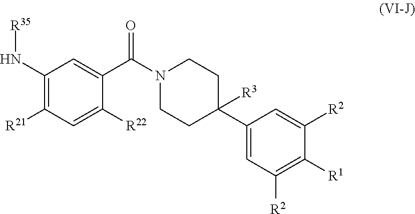

or a pharmaceutically acceptable salt thereof, wherein: R¹ is H, —CN, halogen, C₁-C₄ straight or branched alkyl, —O—(C₃-C₅ cycloalkyl), —O—(C₁-C₄ straight or branched alkyl) wherein the C₃-C₅ cycloalkyl optionally includes an oxygen or nitrogen heteroatom; and when R¹ is not H, —CN or halogen, it is optionally substituted with one or more halogens; each R² is independently H, halogen or C₁-C₄ straight or branched alkyl; R³ is H, —OH, or halogen; R²¹ is cyclobutyl, azetidin-1-yl, or cyclopropyl; R²² is H, halogen, or C₁-C₂ alkyl; R³⁵ is —C(O)—R³⁵¹, —C(O)—NHR³⁵¹, —C(O)—O—R³⁵¹ or S(O)₂R³⁵¹; and R³⁵¹ is C₁-C₆ straight or branched alkyl, cycloalkyl, heterocyclyl, aryl or heteroaryl.

In some aspects of Formula (VI-J), R³ is H or halogen. In some embodiments of Formula (VI-J), R¹ is halogen, —CN or C₁-C₂ haloalkyl. In some embodiments of Formula (VI-J), R²² is C₁-C₂ alkyl. In some embodiments of Formula (VI-J), R²¹ is cyclobutyl and R²² is C₁-C₂ alkyl. In some embodiments of Formula (VI-J), R²¹ is cyclobutyl. In some embodiments of Formula (VI-J), R³ is H or F. In some embodiments of Formula (VI-J), R¹ is-CN. In some embodiments of Formula (VI-J), R¹ is-CF₃. In some embodiments of Formula (VI-J), R²² is H, methyl or ethyl. In some embodiments of Formula (VI-J), R²² is H. In some embodiments of Formula (VI-J), R²² is methyl. In some embodiments of Formula (VI-J), R³⁵ is —C(O)—NHR³⁵¹. In some embodiments of Formula (VI-J), R³⁵¹ is isopropyl, isobutyl, (R)-3-tetrahydrofuranyl, (S)-3-tetrahydrofuranyl, (R)-(tetrahydrofuran-2-yl)methyl, (S)-(tetrahydrofuran-2-yl)methyl, (R)-tetrahydro-2H-pyran-3-yl or (S)-tetrahydro-2H-pyran-3-yl. In some embodiments of Formula (VI-J), R³⁵¹ is (R)-(tetrahydrofuran-2-yl)methyl or (S)-(tetrahydrofuran-2-yl)methyl. In some embodiments of Formula (VI-J), R¹ is-CN, each R² is hydrogen, R³ is H or F, R²¹ is C₃-C₄ cycloalkyl, R²² is H, R³⁵ is —C(O)—NHR³⁵¹ where R³⁵¹ is isopropyl, isobutyl, (R)-3-tetrahydrofuranyl, (S)-3-tetrahydrofuranyl, (R)-(tetrahydrofuran-2-yl)methyl, (S)-(tetrahydrofuran-2-yl)methyl, (R)-tetrahydro-2H-pyran-3-yl, or (S)-tetrahydro-2H-pyran-3-yl. In some embodiments of Formula (VI-J), R³⁵ is —C(O)—O—R³⁵¹. In some embodiments of Formula (VI-J), R³⁵¹ is isopropyl, isobutyl, (R)-3-tetrahydrofuranyl, (S)-3-tetrahydrofuranyl, (R)-(tetrahydrofuran-2-yl)methyl, (S)-(tetrahydrofuran-2-yl)methyl, (R)-tetrahydro-2H-pyran-3-yl, or (S)-tetrahydro-2H-pyran-3-yl. In some embodiments of Formula (VI-J), R¹ is-CN, each R² is H, R³ is H or F, R²¹ is C₃-C₄ cycloalkyl, R²² is H, R³⁵ is —C(O)—O—R³⁵¹ where R³⁵¹ is isopropyl, isobutyl, (R)-3-tetrahydrofuranyl, (S)-3-tetrahydrofuranyl, (R)-(tetrahydrofuran-2-yl)methyl, (S)-(tetrahydrofuran-2-yl)methyl, (R)-tetrahydro-2H-pyran-3-yl, or (S)-tetrahydro-2H-pyran-3-yl. In some embodiments of Formula (VI-J), R³⁵¹ is (R)-3-tetrahydrofuranyl or (S)-3-tetrahydrofuranyl.

In some embodiments of Formula (VI-J), the compound has a structure selected from the group consisting of:

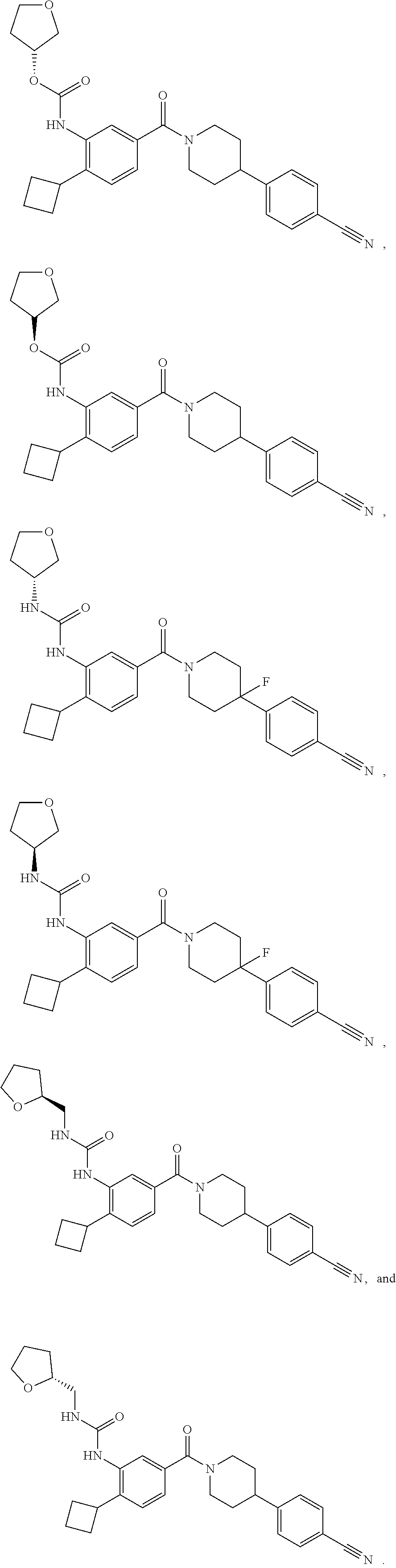

In some embodiments, the FASN inhibitor is a compound of Formula (VII-A) or (VII-B):

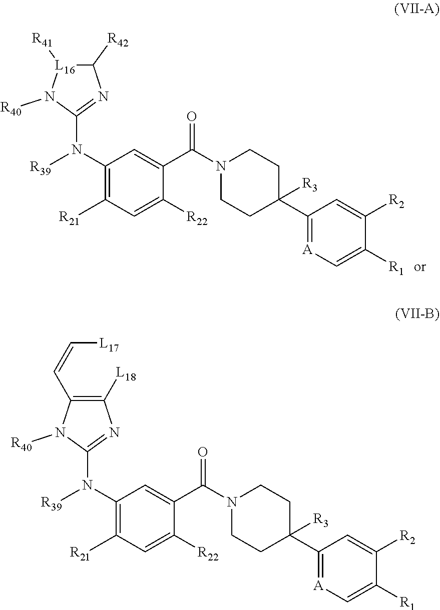

or a pharmaceutically acceptable salt thereof, wherein: L₁₆ is C or N, wherein R₄₁ is absent if L₁₆ is N; L₁₇, L₁₈, and A are each, independently, CH or N; R₁ is hydrogen, cyano, halo, C₁₋₆ alkyl, C₁₋₆ alkoxy, —C(═O)NR₁₃R₁₄, —(CH₂)_{q}C(═O)NR₁₃R₁₄, CF₃, —OCF₃, or —S(═O)₂R₂₀; q is 0, 1, 2, 3, or 4; R₂₀ is hydrogen or C₁₋₆ alkyl, C₁₋₆ alkoxy, or —NR₁₃R₁₄; R₂ is hydrogen, halo, C₁₋₆ alkoxy, or C₁₋₆ alkyl; R₃ is hydrogen, hydroxyl, halo, C₁₋₆ alkyl, or C₁₋₆ alkoxy; R₂₁ and R₂₂ are each, independently, hydrogen, halo, cyano, C₁₋₆ alkyl, C₁₋₆ alkoxy, CF₃, —OCF₃, or —S(═O)₂R₂₀; R₄₀, R₄₂, and R₄₃ are each, independently, hydrogen, C₁₋₆ alkyl, C₁₋₆ alkoxy, —S(═O)₂R₂₀, —C(═O)R, hydroxyalkyl, hydroxyl, —NR₁₃R₁₄, or R₄₁ and R₄₂ taken together with the atoms to which they are attached join together to form a heteroaryl or heterocyclyl; R₄₁ is hydrogen, C₁₋₆ alkyl, C₁₋₆ alkoxy, —S(═O)₂R₂₀, —C(═O)R, hydroxyalkyl, hydroxyl, —NR₁₃R₁₄, R₄₁ is absent if L₁₆ is N, or R₄₁ and R₄₂ taken together with the atoms to which they are attached join together to form a heteroaryl or heterocyclyl; R is hydrogen, C₁₋₆ alkyl, cycloalkyl, aryl, heterocyclyl, heteroaryl, hydroxyalkyl, —NR₁₅R₁₆, or —S(═O)₂R₂₀; R₃₉ is hydrogen, C₁₋₆ alkyl, or C₁₋₆ alkoxy; R₁₃ and R₁₄ are each independently hydrogen, C₁₋₆ alkyl, cycloalkyl, aryl, heterocyclyl, heteroaryl, hydroxyalkyl, alkylamino, —NR₁₅R₁₆, or —S(═O)₂R₂₀; and R₁₅ and R₁₆ are each independently hydrogen, C₁₋₆ alkyl, cycloalkyl, aryl, heterocyclyl, heteroaryl, hydroxyalkyl, or alkylamino.

In some embodiments, R₁ is hydrogen, cyano, C₁₋₆ alkyl, C₁₋₆ alkoxy, or —C(═O)NR₁₃R₁₄. In some embodiments, R₁ is cyano. In some embodiments, R₂ is hydrogen or halo. In some embodiments, R₂ is hydrogen. In some embodiments, R₃ is hydrogen. In some embodiments, R₂₁ and R₂₂ are each independently hydrogen or C₁₋₆ alkyl. In some embodiments, R₂₁ and R₂₂ are each independently C₁₋₆ alkyl. In some embodiments, R₃₉ is hydrogen. In some embodiments, R₄₀ is hydrogen. In some embodiments, L₁₆ is N. In some embodiments, L₁₇ is N. In some embodiments, L₁₈ is CH. In some embodiments, L₁₈ is N. In some embodiments, A is N. In some embodiments, A is CH. In some embodiments, R₄₂ is C₁₋₆ alkyl. In some embodiments, R₄₁ is C₁₋₆ alkyl.

In some embodiments, the FASN inhibitor has the structure of one of the following:

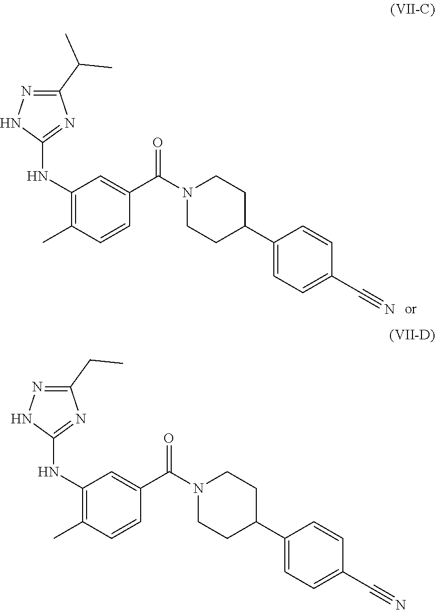 [SMILES: Cc1ccc(C(=O)N2CCC(c3ccc(C#N)cc3)CC2)cc1Nc1nc(C(C)C)n[nH]1 ; CCc1n[nH]c(Nc2cc(C(=O)N3CCC(c4ccc(C#N)cc4)CC3)ccc2C)n1]

or a pharmaceutically acceptable salt thereof.

In some embodiments, the FASN inhibitor is a compound of Formula (VIII-A), (VIII-B), or (VIII-C):

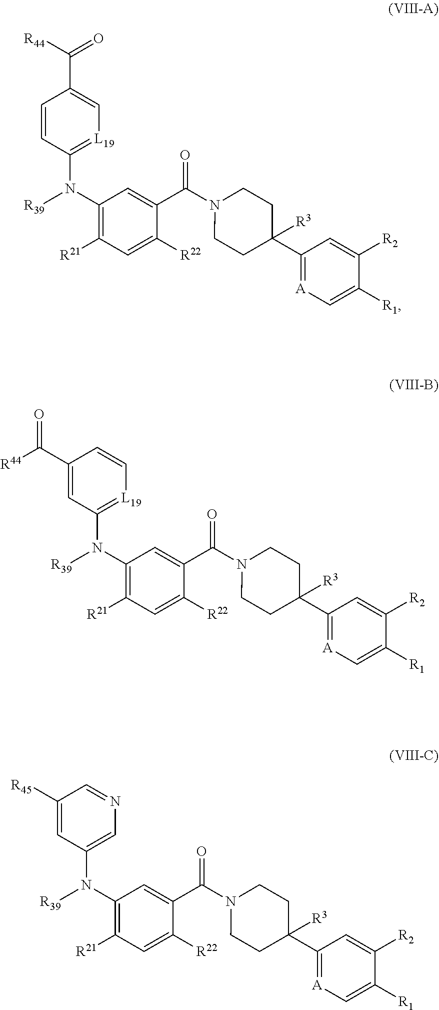

or a pharmaceutically acceptable salt thereof, wherein: L₁₉ and A are each, independently, CH or N; R₁ is hydrogen, cyano, halo, C₁₋₆ alkyl, C₁₋₆ alkoxy, —C(═O)NR₁₃R₁₄, —(CH₂)_{q}C(═O)NR₁₃R₁₄, CF₃, —OCF₃, or —S(═O)₂R₂₀; q is 0, 1, 2, 3, or 4; R₂₀ is hydrogen, C₁₋₆ alkyl, C₁₋₆ alkoxy, or —NR₁₃R₁₄; R₂ is hydrogen, halo, C₁₋₆ alkoxy, or C₁₋₆ alkyl; R₃ is hydrogen, hydroxyl, halo, C₁₋₆ alkyl, or C₁₋₆ alkoxy; R₂₁ and R₂₂ are each independently hydrogen, halo, cyano, C₁₋₆ alkyl, C₁₋₆ alkoxy, CF₃, —OCF₃, or —S(═O)₂R₂₀; R₃₉ is hydrogen, C₁₋₆ alkyl, or C₁₋₆ alkoxy; R₄₄ and R₄₅ are each, independently, hydrogen, C₁₋₆ alkyl, C₁₋₆ alkoxy, cycloalkyl, hydroxyalkyl, aryl, heterocyclyl, heteroaryl, alkylamino, —S(═O)₂R₂₀, —C(═O)R, or —NR₁₃R₁₄; R₁₃ and R₁₄ are each independently hydrogen, C₁₋₆ alkyl, cycloalkyl, aryl, heterocyclyl, heteroaryl, hydroxyalkyl, alkylamino, —NR₁₅R₁₆, or —S(═O)₂R₂₀; and R₁₅ and R₁₆ are each independently hydrogen, C₁₋₆ alkyl, cycloalkyl, aryl, heterocyclyl, heteroaryl, hydroxyalkyl, or alkylamino.

In some embodiments, R₁ is hydrogen, cyano, C₁₋₆ alkyl, C₁₋₆ alkoxy, or —C(═O)NNR₁₃R₁₄. In some embodiments, R₁ is cyano. In some embodiments, R₂ is hydrogen or halo. In some embodiments, R₂ is hydrogen. In some embodiments, R₃ is hydrogen. In some embodiments, R₂₁ and R₂₂ are each independently hydrogen or C₁₋₆ alkyl. In some embodiments, R₂₁ and R₂₂ are each independently C₁₋₆ alkyl. In some embodiments, R₃₉ is hydrogen. In some embodiments, L₁₉ is N. In some embodiments, A is N. In some embodiments, A is CH.

In some embodiments, the compound has the structure:

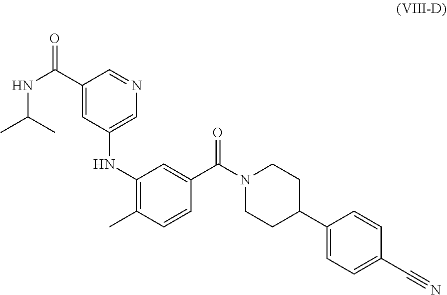 [SMILES: Cc1ccc(C(=O)N2CCC(c3ccc(C#N)cc3)CC2)cc1Nc1cncc(C(=O)NC(C)C)c1]

or a pharmaceutically acceptable salt thereof.

In some embodiments, the FASN inhibitor is a compound of Formula (IX):

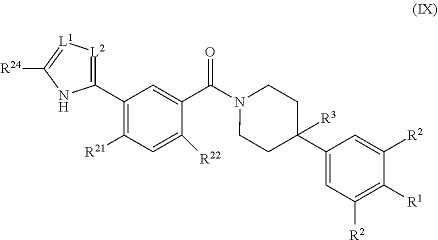

or a pharmaceutically acceptable salt thereof, wherein: R¹ is H, —CN, halogen, C₁-C₄ straight or branched alkyl, —O—(C₃-C₅ cycloalkyl), —O—(C₁-C₄ straight or branched alkyl) wherein: C₃-C₅ cycloalkyl optionally includes an oxygen or nitrogen heteroatom; and when R¹ is not H, —CN or halogen, it is optionally substituted with one or more halogens; each R² is, independently, hydrogen, halogen or C₁-C₄ straight or branched alkyl; R³ is H, —OH, or halogen; R²¹ is H, halogen, C₁-C₄ straight or branched alkyl, C₃-C₅ cycloalkyl wherein the C₃-C₅ cycloalkyl optionally includes an oxygen or nitrogen heteroatom; R²² is H, halogen, or C₁-C₂ alkyl; R²⁴ is H, C₁-C₄ straight or branched alkyl, —(C₁-C₄ alkyl)ₜ-OH, —(C₁-C₄ alkyl)ₜ-Oₜ—(C₃-C₅ cycloalkyl), or —(C₁-C₄ alkyl)ₜ-O—(C₁-C₄ straight or branched alkyl) wherein: t is 0 or 1; the C₃-C₅ cycloalkyl optionally includes an oxygen or nitrogen heteroatom; L¹ is CR²³ or N; L² is CH or N; at least one of L¹ or L² is N; and R²³ is H or C₁-C₄ straight or branched alkyl.

In some aspects of Formula (IX), R²⁴ is C₁-C₄ straight or branched alkyl or —(C₁-C₄ alkyl)ₜ-O—(C₁-C₄ straight or branched alkyl) wherein t is 0 or 1. In some aspects of Formula (IX), R²¹ is halogen, C₁-C₄ straight or branched alkyl, C₃-C₅ cycloalkyl wherein the C₃-C₅ cycloalkyl optionally includes an oxygen or nitrogen heteroatom, —S(O)ᵤ—(C₁-C₄ straight or branched alkyl) wherein u is 0 or 2, or —S(O)ᵤ—(C₃-C₅ cycloalkyl) wherein u is 0 or 2. In some embodiments, R³ is H or halogen. In some embodiments, R¹ is halogen, —CN, or C₁-C₂ haloalkyl. In some embodiments, both L¹ and L² are N. In some embodiments, R²¹ is C₁-C₂ alkyl or C₃-C₅ cycloalkyl and R²² is C₁-C₂ alkyl. In some embodiments, R²¹ is C₃-C₅ cycloalkyl and R²² is C₁-C₂ alkyl. In some embodiments, R²⁴ is —(C₁-C₂ alkyl)ₜ-O—(C₁-C₂ alkyl) wherein t is 0 or 1. In some embodiments, R²¹ is C₃-C₅ cycloalkyl, R²² is C₁-C₂ alkyl and R²⁴ is C₁-C₂ alkyl. In some embodiments, R²¹ is cyclobutyl, R²² is C₁-C₂ alkyl and R²⁴ is C₁-C₂ alkyl. In some embodiments, R²¹ is cyclobutyl. In some embodiments, R³ is H or F. In some embodiments, R¹ is —CN. In some embodiments, R¹ is —CF₃. In some embodiments, R²² is H, methyl, or ethyl. In some embodiments, R²² is H. In some embodiments, R²² is methyl. In some embodiments, R¹ is —CN, each R² is H, R³ is H or F, R²¹ is C₃-C₄ cycloalkyl, R²² is methyl, L¹ and L² are N, and R²⁴ is methyl, ethyl, hydroxymethyl, methoxymethyl, 2-methoxyethyl. In some embodiments, R¹ is —CN, each R² is H, R³ is H or F, R²¹ is C₃-C₄ cycloalkyl, R²² is methyl, L¹ and L² are N, and R²⁴ is methoxy or ethoxy. In some embodiments, R¹ is —CN, each R² is H, R³ is H or F, R²¹ is C₃-C₄ cycloalkyl, R²² is methyl, L¹ is CH, L² is N, and R²⁴ is methyl, ethyl, hydroxymethyl, methoxymethyl, or 2-methoxyethyl. In some embodiments, R¹ is —CN, each R² is H, R³ is H or F, R²¹ is C₃-C₄ cycloalkyl, R²² is methyl, L¹ is N, L² is CH, and R²⁴ is methyl, ethyl, hydroxymethyl, methoxymethyl, or 2-methoxyethyl.

In some embodiments, the compound has a structure selected from the group consisting of:

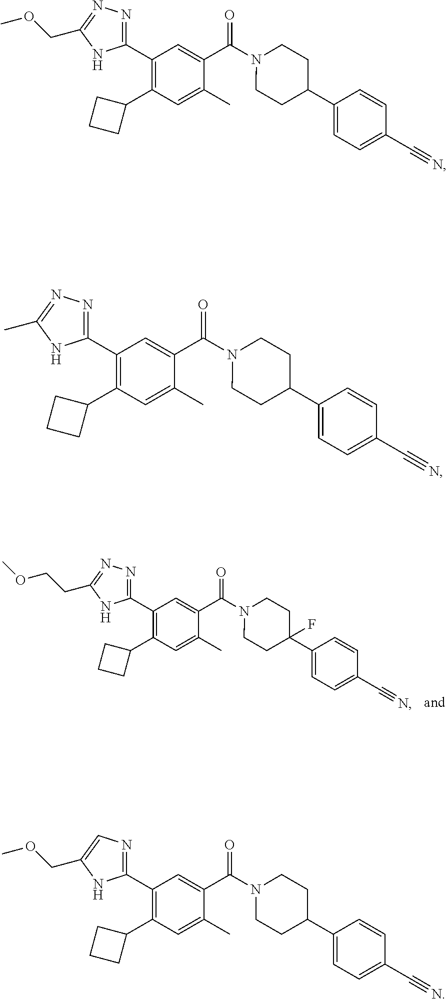 [SMILES: COCc1nnc(-c2cc(C(=O)N3CCC(c4ccc(C#N)cc4)CC3)c(C)cc2C2CCC2)[nH]1 ; Cc1nnc(-c2cc(C(=O)N3CCC(c4ccc(C#N)cc4)CC3)c(C)cc2C2CCC2)[nH]1 ; COCCc1nnc(-c2cc(C(=O)N3CCC(F)(c4ccc(C#N)cc4)CC3)c(C)cc2C2CCC2)[nH]1 ; COCc1cnc(-c2cc(C(=O)N3CCC(c4ccc(C#N)cc4)CC3)c(C)cc2C2CCC2)[nH]1]

In some embodiments, the FASN inhibitor is a compound of Formula (X):

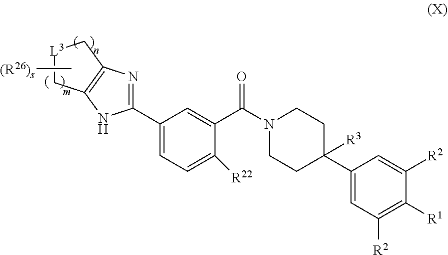

or a pharmaceutically acceptable salt thereof, wherein: R¹ is H, —CN, halogen, C₁-C₄ Straight or branched alkyl, —O—(C₃-C₅ cycloalkyl), —O—(C₁-C₄ Straight or branched alkyl) wherein: the C₃-C₅ cycloalkyl optionally includes an oxygen or nitrogen heteroatom; and when R¹ is not H, —CN or halogen, it is optionally substituted with one or more halogens; each R² is independently hydrogen, halogen or C₁-C₄ Straight or branched alkyl; R³ is H, —OH or halogen; L³ is C(R⁶⁰)₂, O or NR⁵⁰; each R⁶⁰ is independently H, —OH, —CN, —Oₜ—(C₃-C₅ cycloalkyl), —O—(C₁-C₄ Straight or branched alkyl), or —C(O)—NR⁶⁰¹₂ wherein: t is 0 or 1, and the C₃-C₅ cycloalkyl optionally includes an oxygen or nitrogen heteroatom; each R⁵⁰ is independently H, —C(O)—Oₜ—(C₁-C₄ Straight or branched alkyl), —C(O)—Oₜ—(C₃-C₅ cyclic alkyl), —C₃-C₅ cyclic alkyl optionally containing an oxygen or nitrogen heteroatom, —C(O)—NR⁵⁰₂, C₁-C₄ Straight or branched alkyl wherein: t is 0 or 1, and the C₃-C₅ cycloalkyl optionally includes an oxygen or nitrogen heteroatom; n is 1, 2 or 3; m is 1 or 2; R²¹ is H, halogen, C₁-C₄ Straight or branched alkyl, C₃-C₅ cycloalkyl wherein the C₃-C₅ cycloalkyl optionally includes an oxygen or nitrogen heteroatom; R²² is H, halogen, C₁-C₂ alkyl; each R²⁶ is independently-OH, —CN, halogen, C₁-C₄ Straight or branched alkyl, —(C₁-C₄ alkyl)ₜ-Oₜ—(C₃-C₅ cycloalkyl), —(C₁-C₄ alkyl)ₜ-O—(C₁-C₄ straight or branched alkyl), —C(O)—Oₜ—(C₁-C₄ alkyl), or —C(O)—NR⁵⁰¹₂ wherein: t is 0 or 1, and the C₃-C₅ cycloalkyl optionally includes an oxygen or nitrogen heteroatom; s is 0, 1 or 2; each R⁶⁰¹ and R⁵⁰¹ is independently H or C₁-C₄ Straight or branched alkyl; and wherein two of R²⁶, R⁶⁰, R⁵⁰, R⁵⁰¹ and R⁶⁰¹ optionally join to form a ring wherein the two of R²⁶, R⁶⁰, R⁵⁰, R⁵⁰¹ and R⁶⁰¹ may be two R²⁶, two R⁶⁰, two R⁵⁰, two R⁵⁰¹ or two R⁶⁰¹.

In some embodiments, R²¹ is halogen, C₁-C₄ straight or branched alkyl, or C₃-C₅ cycloalkyl. In some embodiments, R³ is H or halogen. In some embodiments, R¹ is —CN or C₁-C₂ haloalkyl. In some embodiments, R³ is H or F. In some embodiments, R¹ is —CN. In some embodiments, R¹ is —CF₃. In some embodiments, n is 1. In some embodiments, n is 2. In some embodiments, m is 1. In some embodiments, m is 2. In some embodiments, R²¹ is C₁-C₂ alkyl or C₃-C₅ cycloalkyl and R²² is C₁-C₂ alkyl. In some embodiments, R²¹ is C₃-C₅ cycloalkyl and R²² is C₁-C₂ alkyl. In some embodiments, n is 2, m is 1, L³ is —N—C(O)—O—(C₁-C₂ alkyl). In some embodiments, L³ is NR⁵⁰; R⁵⁰ is C₁-C₂ alkyl; R²¹ is cyclobutyl; R²² is H or methyl; R³ is H; R¹ is —CN; m is 2 and n is 1 or 2. In some embodiments, n is 2, m is 1, L³ is O and s is 0. In some embodiments, R²² is H, methyl or ethyl. In some embodiments, R²² is methyl. In some embodiments, R²² is H. In some embodiments, R¹ is —CN, each R² is H, R³ is H or F, R²¹ is C₃-C₄ cycloalkyl, R²² is methyl, n is 2 and L³ is NR⁵⁰ where R⁵⁰ is methyl or ethyl. In some embodiments, R¹ is —CN, each R² is H, R³ is H or F, R²¹ is C₃-C₄ cycloalkyl, R²² is methyl, n is 2 and L³ is O. In some embodiments, the compound has a structure selected from the group consisting of:

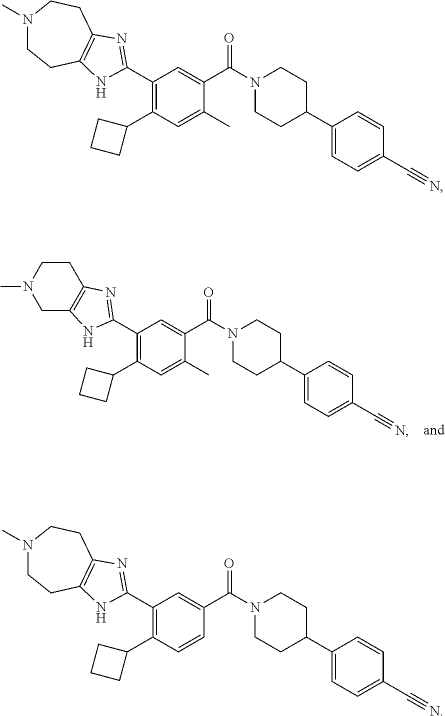 [SMILES: Cc1cc(C2CCC2)c(-c2nc3c([nH]2)CCN(C)CC3)cc1C(=O)N1CCC(c2ccc(C#N)cc2)CC1 ; Cc1cc(C2CCC2)c(-c2nc3c([nH]2)CN(C)CC3)cc1C(=O)N1CCC(c2ccc(C#N)cc2)CC1 ; CN1CCc2nc(-c3cc(C(=O)N4CCC(c5ccc(C#N)cc5)CC4)ccc3C3CCC3)[nH]c2CC1]

In some embodiments, the FASN inhibitor is a compound of Formula (XI):

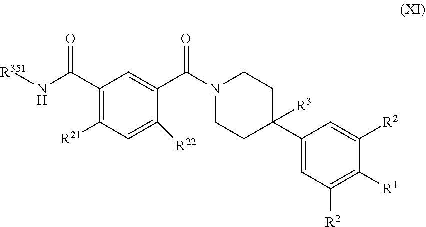

or a pharmaceutically acceptable salt thereof, wherein: R¹ is H, —CN, halogen, C₁-C₄ straight or branched alkyl, —O—(C₃-C₅s cycloalkyl), —O—(C₁-C₄ straight or branched alkyl) wherein: the C₃-C₅ cycloalkyl optionally includes an oxygen or nitrogen heteroatom; and when R¹ is not H, —CN or halogen, it is optionally substituted with one or more halogens; each R² is independently H, halogen or C₁-C₄ straight or branched alkyl; R³ is H, —OH, or halogen; R²¹ is cyclobutyl, azetidin-1-yl, or cyclopropyl; R²² is H, halogen, C₁-C₂ alkyl; and R³⁵¹ is C₁-C₂ alkyl or C₂—O—(C₁ or C₂ alkyl).

In some embodiments, R³ is H or halogen. In some embodiments, R¹ is halogen, —CN or C₁-C₂ haloalkyl. In some embodiments, R²¹ is C₃-C₄ cycloalkyl and R²² is C₁-C₂ alkyl. In some embodiments, R²¹ is cyclobutyl and R²² is C₁-C₂ alkyl. In some embodiments, R²¹ is cyclobutyl. In some embodiments, R³ is H or F. In some embodiments, R¹ is —CN. In some embodiments, R¹ is —CF₃. In some embodiments, R²² is H, methyl or ethyl. In some embodiments, R²² is H. In some embodiments, R²² is methyl. In some embodiments, R¹ is —CN, each R² is H, R³ is H or F, R²¹ is cyclobutyl, R²² is methyl and R³⁵¹ is methyl or ethyl.

In some embodiments, the compound has a structure selected from the group consisting of:

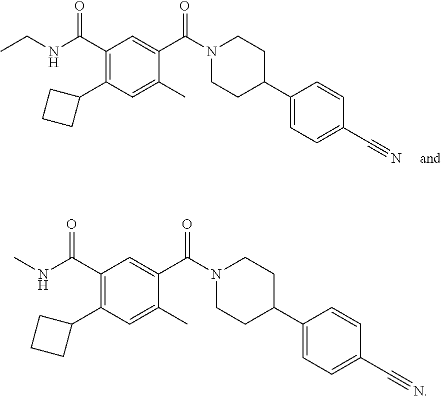 [SMILES: CCNC(=O)c1cc(C(=O)N2CCC(c3ccc(C#N)cc3)CC2)c(C)cc1C1CCC1 ; CNC(=O)c1cc(C(=O)N2CCC(c3ccc(C#N)cc3)CC2)c(C)cc1C1CCC1]

In some embodiments, the FASN inhibitor is a compound of Formula (XII):

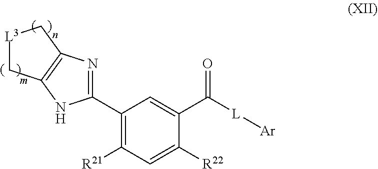

or a pharmaceutically acceptable salt thereof, wherein: L³ is —CH₂—, —CHR⁵⁰—, —O—, —NR⁵⁰—, —NC(O)R⁵⁰- or —NC(O)OR⁵⁰—, wherein R⁵⁰ is C₁-C₆ alkyl, C₃-C₅ cycloalkyl, or 4- to 6-membered heterocycle; n is 1, 2, or 3; m is 1 or 2 with the proviso that n+m≥3; L-Ar is

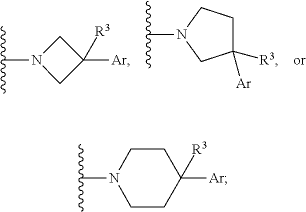

### Ar is

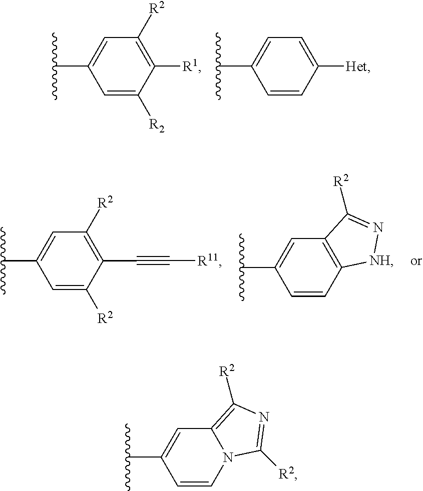

with the proviso that when L-Ar is

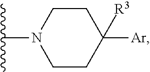

### Ar is not

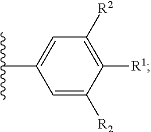

Het is a 5- to 6-membered heteroaryl; R¹ is H, —CN, halogen, C₁-C₄ alkyl, —O—(C₃-C₅ cycloalkyl), —O-(4- to 6-membered heterocycle) or —O—(C₁-C₄ alkyl), wherein when R¹ is not H, —CN or halogen, R¹ is optionally substituted with one or more halogens; each R² is independently hydrogen, halogen or C₁-C₄ alkyl; R³ is H or F; R¹¹ is H or —CH₃; R²¹ is H, halogen, C₁-C₄ alkyl, C₃-C₅ cycloalkyl or a 4- to 6-membered heterocycle; and R²² is H, halogen, or C₁-C₂ alkyl.

As noted above, each of the C₁-C₂ alkyl, C₁-C₄ alkyl, C₁-C₆ alkyl, C₃-C₅ cycloalkyl, 4- to 6-membered heterocycle and 5- to 6-membered heteroaryl moieties may be optionally substituted.

Accordingly, the present disclosure provides for compounds of Formula (XII) wherein: L³ is —CH₂—, CHR⁵⁰, —O—, —NR⁵⁰—, —NC(O)R⁵⁰— or —NC(O)OR⁵⁰—, wherein R⁵⁰ is optionally substituted C₁-C₆ alkyl, optionally substituted C₃-C₅ cycloalkyl or optionally substituted 4- to 6-membered heterocycle; n is 1, 2 or 3; m is 1 or 2 with the proviso that n+m≥3; L-Ar is

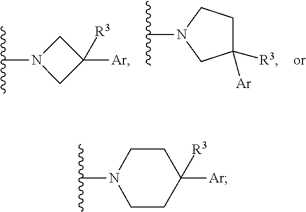

### Ar is

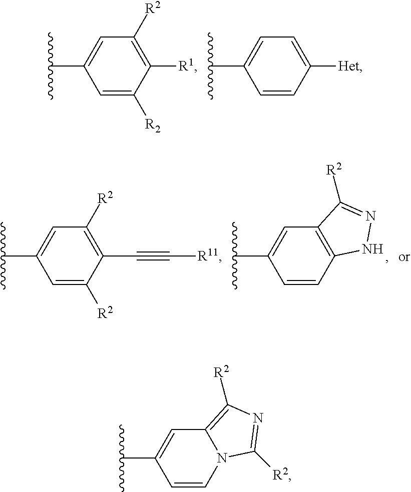

with the proviso that when L-Ar is

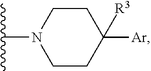

### Ar is not

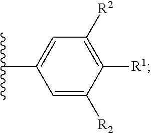

Het is an optionally substituted 5- to 6-membered heteroaryl; R¹ is H, —CN, halogen, optionally substituted C₁-C₄ alkyl, —O-(optionally substituted C₃-C₅ cycloalkyl), —O-(optionally substituted 4- to 6-membered heterocycle) or —O-(optionally substituted C₁-C₄ alkyl), wherein when R¹ is not H, —CN or halogen, R¹ is optionally substituted with one or more halogens; each R² is independently hydrogen, halogen or optionally substituted C₁-C₄ alkyl; R³ is H or F; R¹¹ is H or —CH₃; R²¹ is H, halogen, optionally substituted C₁-C₄ alkyl, optionally substituted C₃-C₅ cycloalkyl or an optionally substituted 4- to 6-membered heterocycle; and R²² is H, halogen or optionally substituted C₁-C₂ alkyl.

In some embodiments, L-Ar is

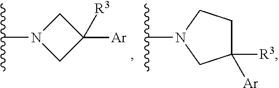

### and Ar is

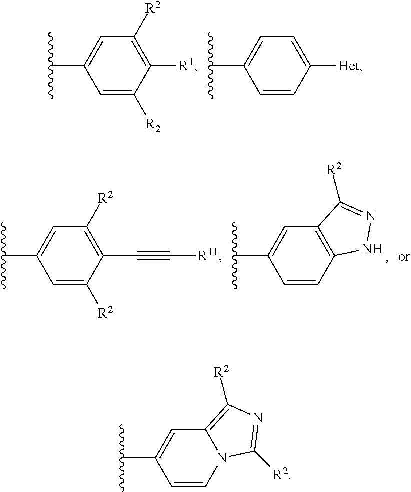

In some embodiments, L-Ar is

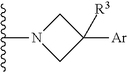

### and Ar is

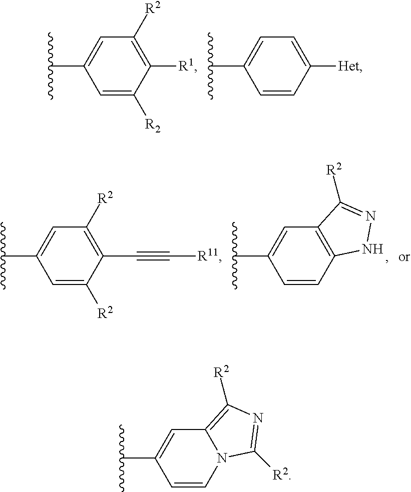

In some embodiments, R¹ is H, —CN, —C₁-C₄ alkyl, —O—(C₃-C₅ cycloalkyl), —O-(4- to 6-membered heterocycle) or —O—(C₁-C₄ alkyl) wherein when R¹ is not H or —CN, R¹ is optionally substituted with one or more halogens. In some embodiments, R¹ is halogen, —CN or C₁-C₂ haloalkyl. In some embodiments, R¹ is —CN or C₁-C₂ haloalkyl. In some embodiments, R¹ is —CN. In some embodiments, R¹ is —Cl. In some embodiments, R² is H. In some embodiments, R²¹ is halogen, C₁-C₄ alkyl, C₃-C₅ cycloalkyl or 4- to 6-membered heterocycle. In some embodiments, R²¹ is C₁-C₂ alkyl or C₃-C₅ cycloalkyl. In some embodiments, R²¹ is C₃-C₅ cycloalkyl. In some embodiments, R²² is H or C₁-C₂ alkyl. In some embodiments, R²² is H. In some embodiments, R²² is C₁-C₂ alkyl. In some embodiments, R²² is —CH₃. In some embodiments, L³ is —N(CH₃)—. In some embodiments, n is 2 and m is 2. In some embodiments, n is 1 or 2. In some embodiments, n is 1 and m is 2. In some embodiments, R²¹ is C₁-C₂ alkyl or C₃-C₅ cycloalkyl and R²² is C₁-C₂ alkyl. In some embodiments, R²¹ is C₁-C₂ alkyl or C₃-C₅ cycloalkyl and R²² is H or C₁-C₂ alkyl. In some embodiments, R²¹ is C₃-C₅ cycloalkyl and R²² is H or C₁-C₂ alkyl. In some embodiments, R²¹ is C₃-C₅ cycloalkyl and R²² is H or —CH₃.

In some embodiments, the FASN inhibitor is a compound of Formula (XIII):

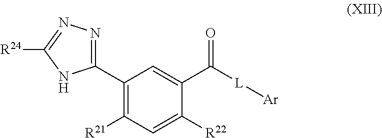

or a pharmaceutically acceptable salt thereof, wherein: L-Ar is

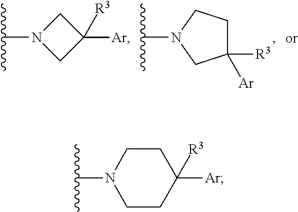

### Ar is

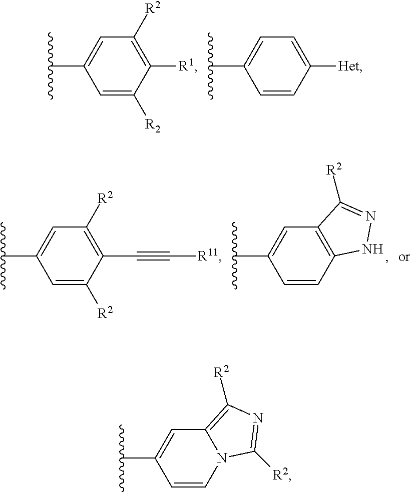

with the proviso that when L-Ar is

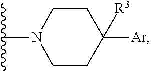

### Ar is not

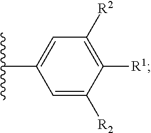

Het is a 5- to 6-membered heteroaryl; R¹ is H, —CN, halogen, C₁-C₄ alkyl, —O—(C₃-C₅ cycloalkyl), —O-(4- to 6-membered heterocycle) or —O—(C₁-C₄ alkyl), wherein when R¹ is not H, —CN or halogen, R¹ is optionally substituted with one or more halogens; each R² is independently hydrogen, halogen or C₁-C₄ alkyl; R³ is H or F; R¹¹ is H or —CH₃; R²¹ is H, halogen, C₁-C₄ alkyl, C₃-C₅ cycloalkyl or 4- to 6-membered heterocycle; R²² is H, halogen, or C₁-C₂ alkyl; and R²⁴ is H, C₁-C₄ alkyl, —(C₁-C₄ alkyl)-OH, —(C₁-C₄ alkyl)ₜ-NR²⁴¹₂, —(C₁-C₄ alkyl)ₜ-Oₜ—(C₃-C₅ cycloalkyl), —(C₁-C₄ alkyl)ₜ-Oₜ-(4- to 6-membered heterocycle) or —(C₁-C₄ alkyl)ₜ-O—(C₁-C₄ alkyl), wherein: each t is independently 0 or 1; and each R²⁴¹ is independently H or C₁-C₂ alkyl.

As noted above, each of the C₁-C₂ alkyl, C₁-C₄ alkyl, C₃-C₅ cycloalkyl, 4- to 6-membered heterocycle and 5- to 6-membered heteroaryl moieties may be optionally substituted. Accordingly, the present disclosure provides for compounds of Formula (XIII) wherein: L-Ar is

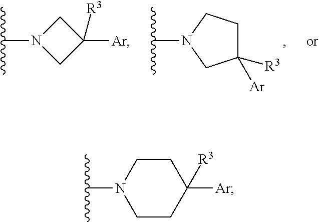

### Ar is

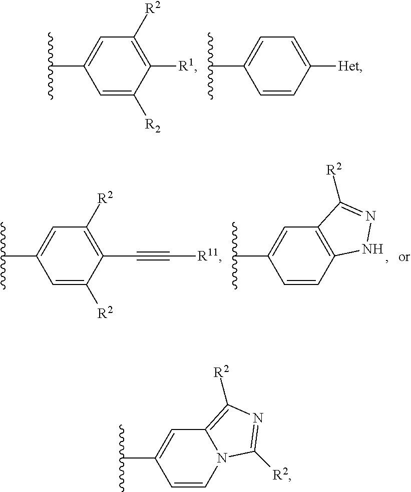

with the proviso that when L-Ar is

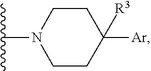

### Ar is not

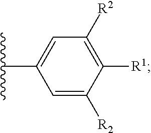

Het is an optionally substituted 5- to 6-membered heteroaryl; R¹ is H, —CN, halogen, optionally substituted C₁-C₄ alkyl, —O-(optionally substituted C₃-C₅ cycloalkyl), —O-(optionally substituted 4- to 6-membered heterocycle) or —O− (optionally substituted C₁-C₄ alkyl), wherein when R¹ is not H, —CN or halogen R¹ is optionally substituted with one or more halogens; each R² is independently hydrogen, halogen or optionally substituted C₁-C₄ alkyl; R³ is H or F; R¹¹ is H or —CH₂; R²¹ is H, halogen, optionally substituted C₁-C₄ alkyl, optionally substituted C₃-C₅ cycloalkyl or optionally substituted 4- to 6-membered heterocycle; R²² is H, halogen or optionally substituted C₁-C₂ alkyl; and R²⁴ is H, optionally substituted C₁-C₄ alkyl, (optionally substituted C₁-C₄ alkyl)-OH, -(optionally substituted C₁-C₄ alkyl)ₜ-NR²⁴¹₂, -(optionally substituted C₁-C₄ alkyl)ₜ-Oₜ-(optionally substituted C₃-C₅ cycloalkyl), -(optionally substituted C₁-C₄ alkyl)ₜ-Oₜ-(optionally substituted 4- to 6-membered heterocycle) or -(optionally substituted C₁-C₄ alkyl)ₜ-O-(optionally substituted C₁-C₄ alkyl), wherein: t is 0 or 1; and R²⁴¹ is H or optionally substituted C₁-C₂ alkyl.

In some embodiments, L-Ar is

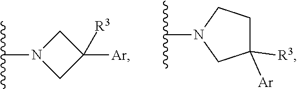

### and Ar is

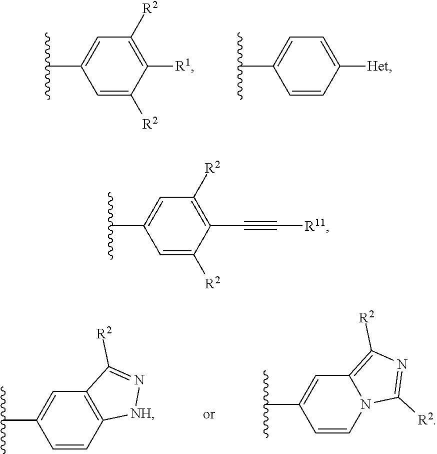

In some embodiments, L-AR is

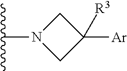

### and Ar is

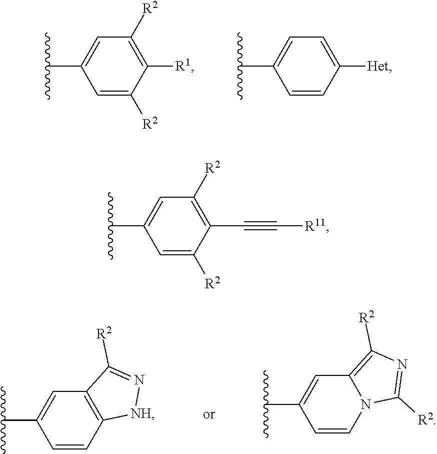

In some embodiments, Ar is

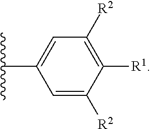

In some embodiments, R¹ is halogen, —CN or C₁-C₂ haloalkyl. In some embodiments, R¹ is —CN. In some embodiments, R² is H. In some embodiments, R²¹ is halogen, C₁-C₄ alkyl, C₃-C₅ cycloalkyl or 4- to 6-membered heterocycle. In some embodiments, R²¹ is H, C₁-C₄ alkyl, C₃-C₅ cycloalkyl or 4- to 6-membered heterocycle. In some embodiments, R²¹ is C₁-C₂ alkyl or C₃-C₅ cycloalkyl. In some embodiments, R²¹ is C₁-C₂ alkyl. In some embodiments, R²¹ is C₃-C₅ cycloalkyl. In some embodiments, R²² is H or C₁-C₂ alkyl. In some embodiments, R²² is H. In some embodiments, R²² is C₁-C₂ alkyl. In some embodiments, R²² is —CH₃. In some embodiments, R²⁴ is C₁-C₄ alkyl or —(C₁-C₄ alkyl)ₜ-O—(C₁-C₄ alkyl). In some embodiments, R²⁴ is —(C₁-C₂ alkyl)ₜ-O—(C₁-C₂ alkyl). In some embodiments, R²⁴ is C₁-C₄ alkyl or —(C₁-C₄ alkyl)ₜ-O—(C₁-C₄ alkyl) wherein t is 0 or 1. In some embodiments, R²¹ is C₁-C₂ alkyl or C₃-C₅ cycloalkyl and R²² is H or C₁-C₂ alkyl. In some embodiments, R²¹ is C₁-C₂ alkyl or C₃-C₅ cycloalkyl and R²² is C₁-C₂ alkyl. In some embodiments, R²¹ is C₁-C₂ alkyl or C₃-C₅ cycloalkyl and R²² is-CH₃. In some embodiments, R²¹ is C₁-C₂ alkyl or C₃-C₅ cycloalkyl and R²² is H. In some embodiments, R²¹ is C₃-C₅ cycloalkyl and R²² is H or C₁-C₂ alkyl. In some embodiments, R²¹ is C₃-C₅ cycloalkyl and R²² is H or —CH₃. In some embodiments, R²¹ is C₃-C₅ cycloalkyl and R²² is C₁-C₂ alkyl. In some embodiments, R²¹ is C₃-C₅ cycloalkyl and R²² is-CH₃. In some embodiments, R²¹ is C₃-C₅ cycloalkyl and R²² is H. In some embodiments, R²⁴ is —(C₁-C₂ alkyl)ₜ-O—(C₁-C₂ alkyl) and wherein t is 0 or 1. In some embodiments, R¹ is —CN and R² is H.

In some embodiments, the FASN inhibitor is a compound of Formula (XIV):

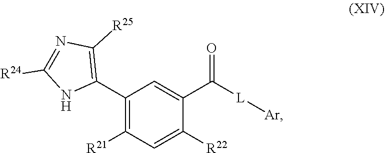

or a pharmaceutically acceptable salt thereof, wherein: L-Ar is

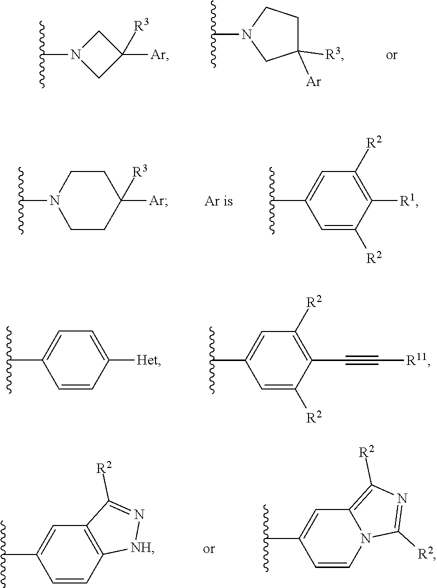

Het is a 5- to 6-membered heteroaryl; R¹ is H, —CN, halogen, C₁-C₄ alkyl, —O—(C₃-C₅ cycloalkyl), —O-(4- to 6-membered heterocycle) or —O—(C₁-C₄ alkyl), wherein when R¹ is not H, —CN or halogen, R¹ is optionally substituted with one or more halogens; each R² is independently hydrogen, halogen or C₁-C₄ alkyl; R³ is H or F; R¹¹ is H or —CH₃; R²¹ is H, halogen, C₁-C₄ alkyl, C₃-C₅ cycloalkyl or 4- to 6-membered heterocycle; R²² is H, halogen, or C₁-C₂ alkyl; R²⁴ is H, —CN, —(C₁-C₄ alkyl)-CN, C₁-C₄ alkyl, —(C₁-C₄ alkyl)-OH, —(C₁-C₄ alkyl)-NR²⁴¹₂, —(C₁-C₄ alkyl)ₜ-Oᵤ—(C₃-C₆ cycloalkyl), —(C₁-C₄ alkyl)ₜ-Oᵤ-(4- to 6-membered heterocycle) or —(C₁-C₄ alkyl)-O—(C₁-C₄ alkyl), wherein: t is 0 or 1; u is 0 or 1; with the proviso that when u is 1, t is 1; and each R²⁴¹ is independently H or C₁-C₂ alkyl; and R²⁵ is halogen, —CN, —(C₁-C₄ alkyl)-CN, C₁-C₂ alkyl or cyclopropyl.

As noted above, each of the C₁-C₂ alkyl (i.e., methyl and ethyl), cyclopropyl, C₁-C₂ alkyl, C₁-C₄ alkyl, C₃-C₅ cycloalkyl, C₃-C₆ cycloalkyl, 4- to 6-membered heterocycle and 5- to 6-membered heteroaryl moieties may be optionally substituted. Accordingly, the present disclosure provides for compounds wherein: L-Ar is

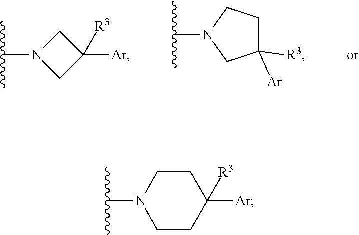

### Ar is

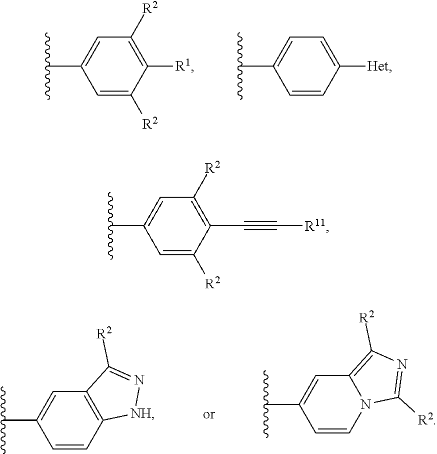

Het is an optionally substituted 5- to 6-membered heteroaryl; R¹ is H, —CN, halogen, optionally substituted C₁-C₄ alkyl, —O-(optionally substituted C₃-C₅ cycloalkyl), —O-(optionally substituted 4- to 6-membered heterocycle) or —O-(optionally substituted C₁-C₄ alkyl), wherein when R¹ is not H, —CN or halogen, R¹ is optionally substituted with one or more halogens; each R² is independently hydrogen, halogen or optionally substituted C₁-C₄ alkyl; R³ is H or F; R¹¹ is H or —CH₃; R²¹ is H, halogen, optionally substituted C₁-C₄ alkyl, optionally substituted C₃-C₅ cycloalkyl or optionally substituted 4- to 6-membered heterocycle; R²² is H, halogen or optionally substituted C₁-C₂ alkyl; R²⁴ is H, —CN, -(optionally substituted C₁-C₄ alkyl)-CN, optionally substituted C₁-C₄ alkyl, -(optionally substituted C₁-C₄ alkyl)-OH, -(optionally substituted C₁-C₄ alkyl)-NR²⁴¹₂, -(optionally substituted C₁-C₄ alkyl)ₜ-Oᵤ-(optionally substituted C₃-C₆ cycloalkyl), -(optionally substituted C₁-C₄ alkyl)ₜ-Oᵤ-(optionally substituted 4- to 6-membered heterocycle) or -(optionally substituted C₁-C₄ alkyl)-O-(optionally substituted C₁-C₄ alkyl), wherein: t is 0 or 1; u is 0 or 1; with the proviso that when u is 1, t is 1; and R²⁴¹ is H or optionally substituted C₁-C₂ alkyl; and R²⁵ is halogen, —CN, -(optionally substituted C₁-C₄ alkyl)-CN, optionally substituted methyl, optionally substituted ethyl or optionally substituted cyclopropyl.

In some embodiments, when L-Ar is

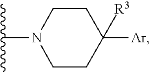

### Ar is not

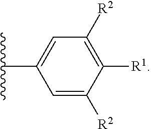

In some embodiments, L-Ar is

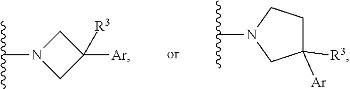

### and Ar is

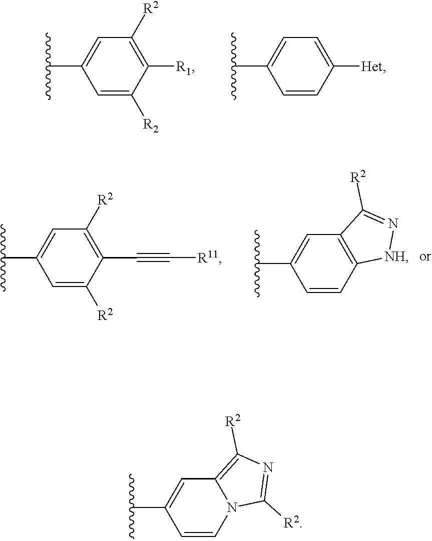

In some embodiments, L-Ar is

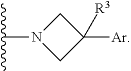

### and Ar is

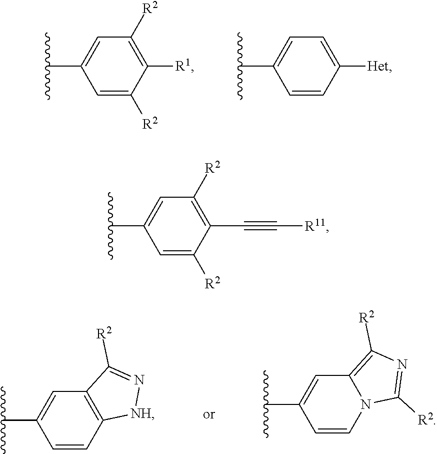

In some embodiments, Ar is

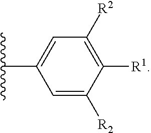

In some embodiments, R¹ is halogen, —CN or C₁-C₂ haloalkyl. In some embodiments, R¹ is —CN. In some embodiments, R² is H. In some embodiments, R²¹ is halogen, C₁-C₄ alkyl or C₃-C₅ cycloalkyl. In some embodiments, R²¹ is C₁-C₄ alkyl or C₃-C₅ cycloalkyl. In some embodiments, R²¹ is C₁-C₂ alkyl or C₃-C₅ cycloalkyl. In some embodiments, R²¹ is C₁-C₂ alkyl. In some embodiments, R²¹ is —CH₃. In some embodiments, R²² is H or C₁-C₂ alkyl. In some embodiments, R²² is H or —CH₃. In some embodiments, R²² is —CH₃. In some embodiments, R²⁴ is H, —CN, —(C₁-C₄ alkyl)-CN, C₁-C₄ alkyl, —(C₁-C₄ alkyl)-OH, —(C₁-C₄ alkyl)-NR²⁴¹₂, —(C₁-C₄ alkyl)ₜ-Oᵤ—(C₃-C₆ cycloalkyl), —(C₁-C₄ alkyl)ₜ-Oᵤ-(4- to 6-membered heterocycle) or —(C₁-C₄ alkyl)-O—(C₁-C₄ alkyl). In some embodiments, R²⁴ is H, C₁-C₄ alkyl, —(C₁-C₄ alkyl)-OH, —(C₁-C₄ alkyl)-NR²⁴¹₂, —(C₁-C₄ alkyl)ₜ-Oᵤ—(C₃-C₆ cycloalkyl), —(C₁-C₄ alkyl)ₜ-Oᵤ—(4- to 6-membered heterocycle) or —(C₁-C₄ alkyl)-O—(C₁-C₄ alkyl). In some embodiments, R²⁴ is C₁-C₄ alkyl or —(C₁-C₄ alkyl)-O—(C₁-C₄ alkyl). In some embodiments, R²⁴ is —(C₁-C₂ alkyl)-O—(C₁-C₂ alkyl). In some embodiments, R²⁴ is —CH₂—O—CH₃. In some embodiments, R²⁴ is C₁-C₂ alkyl. In some embodiments, R²⁴ is —CH₃. In some embodiments, R²⁴ is C₃-C₆ cycloalkyl. In some embodiments, R²⁴ is C₃-C₅ cycloalkyl. In some embodiments, R²⁴ is —CN or —(C₁-C₂ alkyl)-CN. In some embodiments, R²⁴ is —CN. In some embodiments, R²⁴ is —(C₁-C₂ alkyl)-CN. In some embodiments, R²⁴ is H, —CH₃, —CH₂OH, —CH₂OCH₃, —(CH₂)₂OH, —(CH₂)₂OCH₃ or —(CH₂)₂N(CH₃)₂. In some embodiments, R²⁴ is methyl, isopropyl, cyclopropyl, —CN, or —(C₁-C₂ alkyl)-CN. In some embodiments, R²⁴ is substituted with one or more substituents selected from C₁-C₂ alkyl, oxo, —CN, halogen, alkanoyl, alkoxycarbonyl, —OH and C₁-C₂ alkoxy. In some embodiments, R²⁴ is substituted with one or more substituents selected from methyl, —F, methoxy, —C(═O)CH₃ and —C(═O)—OCH₃. In some embodiments, R²⁴ is substituted with two substituents that are the same or different. In some embodiments, R²⁴ is substituted with three substituents that are the same or different. In some embodiments, R²⁵ is halogen, —CN, C₁-C₂ alkyl or cyclopropyl. In some embodiments, R²⁵ is halogen, C₁-C₂ alkyl or cyclopropyl. In some embodiments, R²⁵ is —CN, —Cl, or —CH₃. In some embodiments, R²⁵ is —Cl. In some embodiments, R²⁵ is —CH₃. In some embodiments, R²⁵ is substituted with one or more substituents selected from —OH, halogen, C₁-C₂ alkyl and alkylcarbonyloxy. In some embodiments, R²⁵ is substituted with one or more substituents selected from —F, methyl, and —O—C(═O)—CH₃. In some embodiments, R²⁵ is substituted with two substituents that are the same or different. In some embodiments, R²⁵ is substituted with three substituents that are the same or different. In some embodiments, R²⁴ is C₁-C₄ alkyl, —(C₁-C₄ alkyl)-CN or —(C₃-C₆ cycloalkyl). In some embodiments, R²⁴ is-CN, —(C₁-C₂ alkyl)-CN, —(C₃-C₆ cycloalkyl) or methyl. In some embodiments, R²⁵ is is halogen, methyl, ethyl or cyclopropyl. In some embodiments, R²⁵ is halogen, —CN, methyl, ethyl or cyclopropyl. In some embodiments, R²¹ is C₁-C₂ alkyl or C₃-C₆ cycloalkyl and R²² is H or —CH₃. In some embodiments, R²¹ is C₁-C₂ alkyl or C₃-C₆ cycloalkyl, R²² is H or —CH₃, R²⁴ is-CH₂—O—CH₃ and R²⁵ is —CH₃. In some embodiments, R²¹ is-CH₃ and R²² is H. In some embodiments, R¹ is-CN and R² is H. In some embodiments, R²¹ is C₁-C₂ alkyl or C₃-C₆ cycloalkyl and R²² is H or C₁-C₂ alkyl. In some embodiments, R²¹ is C₁-C₂ alkyl or C₃-C₆ cycloalkyl, R²² is H or C₁-C₂ alkyl, R²⁴ is-CH₂—O—CH₃ and R²⁵ is —CH₃. In some embodiments, R²¹ is C₁-C₂ alkyl and R²² is H.

In some embodiments of Formula (XIV), the FASN inhibitor is a compound of Formula (XIV-B):

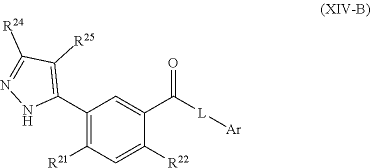

or a pharmaceutically acceptable salt thereof, wherein: L-Ar is

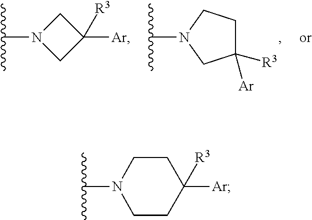

### Ar is

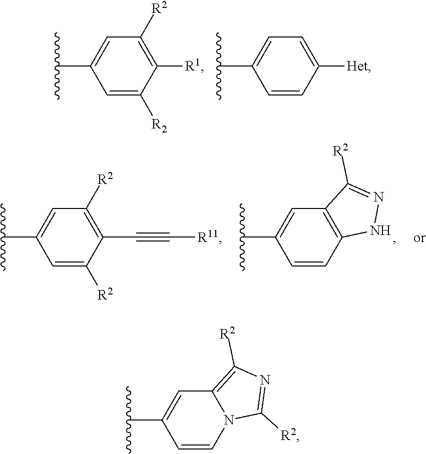

Het is a 5- to 6-membered heteroaryl; R¹ is H, —CN, halogen, C₁-C₄ alkyl, —O—(C₃-C₅ cycloalkyl), —O-(4- to 6-membered heterocycle) or —O—(C₁-C₄ alkyl), wherein when R¹ is not H, —CN or halogen, R¹ is optionally substituted with one or more halogens; each R² is independently hydrogen, halogen or C₁-C₄ alkyl; R³ is H or F; R¹¹ is H or —CH₃; R²¹ is H, halogen, C₁-C₄ alkyl, C₃-C₅ cycloalkyl or 4- to 6-membered heterocycle; R²² is H, halogen or C₁-C₂ alkyl; and each R²⁴ and R²⁵ is independently H, halogen, —CN, —(C₁-C₄ alkyl)-CN, C₁-C₄ alkyl, —(C₁-C₄ alkyl)-OH, —(C₁-C₄ alkyl)-NR²⁴¹₂, —(C₁-C₄ alkyl)ₜ-Oᵤ—(C₃-C₅ cycloalkyl), —(C₁-C₄ alkyl)ₜ-Oᵤ-(4- to 6-membered heterocycle) or —(C₁-C₄ alkyl)ₜ-O—(C₁-C₄ alkyl), wherein: each t is independently 0 or 1; each u is independently 0 or 1; and each R²⁴¹ is independently H or C₁-C₂ alkyl, wherein the compound is not:

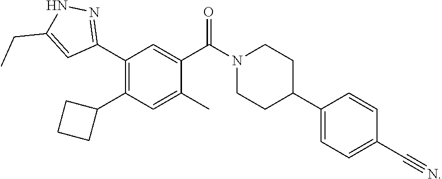 [SMILES: CCc1cc(-c2cc(C(=O)N3CCC(c4ccc(C#N)cc4)CC3)c(C)cc2C2CCC2)n[nH]1]

As noted above, each of the C₁-C₂ alkyl, C₁-C₄ alkyl, C₃-C₅ cycloalkyl, 4- to 6-membered heterocycle and 5- to 6-membered heteroaryl moieties may be optionally substituted. Accordingly, the present disclosure provides for compounds of Formula (XIV-B) wherein: L-Ar is

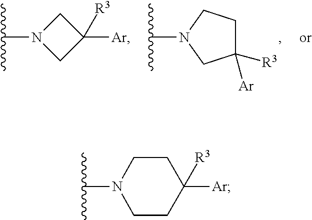

### Ar is

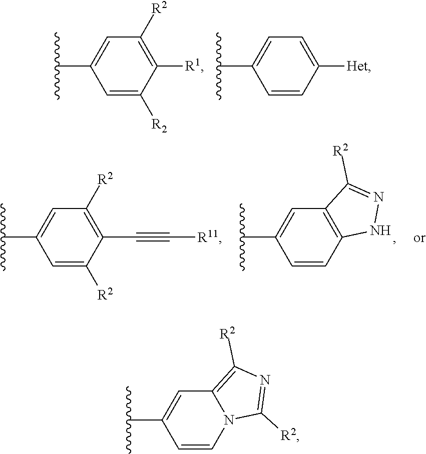

Het is an optionally substituted 5- to 6-membered heteroaryl; R¹ is H, —CN, halogen, optionally substituted C₁-C₄ alkyl, —O-(optionally substituted C₃-C₅ cycloalkyl), —O-(optionally substituted 4- to 6-membered heterocycle) or —O-(optionally substituted C₁-C₄ alkyl), wherein when R¹ is not H, —CN or halogen, R¹ is optionally substituted with one or more halogens; each R² is independently hydrogen, halogen or optionally substituted C₁-C₄ alkyl; R³ is H or F; R¹¹ is H or —CH₃; R²¹ is H, halogen, optionally substituted C₁-C₄ alkyl, optionally substituted C₃-C₅ cycloalkyl or optionally substituted 4- to 6-membered heterocycle; R²² is H, halogen or optionally substituted C₁-C₂ alkyl; and each R²⁴ and R²⁵ is independently H, halogen, —CN, -(optionally substituted C₁-C₄ alkyl)-CN, optionally substituted C₁-C₄ alkyl, -(optionally substituted C₁-C₄ alkyl)-OH, —(optionally substituted C₁-C₄ alkyl)-NR²⁴¹₂, -(optionally substituted C₁-C₄ alkyl)ₜ-Oᵤ-(optionally substituted C₃-C₅ cycloalkyl), -(optionally substituted C₁-C₄ alkyl)ₜ-Oᵤ-(optionally substituted 4- to 6-membered heterocycle) or -(optionally substituted C₁-C₄ alkyl)ₜ-O-(optionally substituted C₁-C₄ alkyl), wherein: t is 0 or 1; u is 0 or 1; and R²⁴¹ is H or optionally substituted C₁-C₂ alkyl, wherein the compound is not:

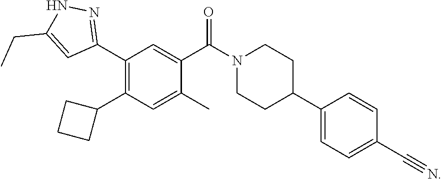 [SMILES: CCc1cc(-c2cc(C(=O)N3CCC(c4ccc(C#N)cc4)CC3)c(C)cc2C2CCC2)n[nH]1]

In some embodiments, when L-Ar is

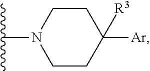

### Ar is not

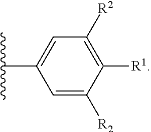

In some embodiments, L-Ar is

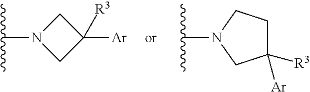

### and Ar is

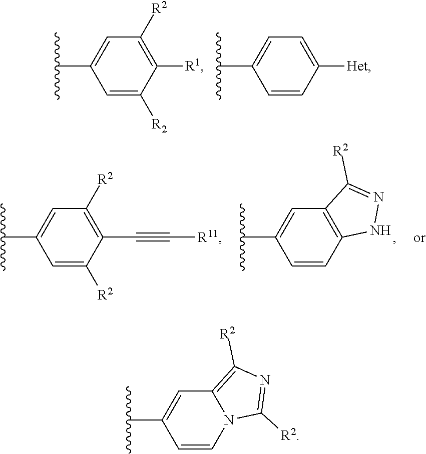

In some embodiments, L-Ar is

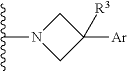

### and Ar is

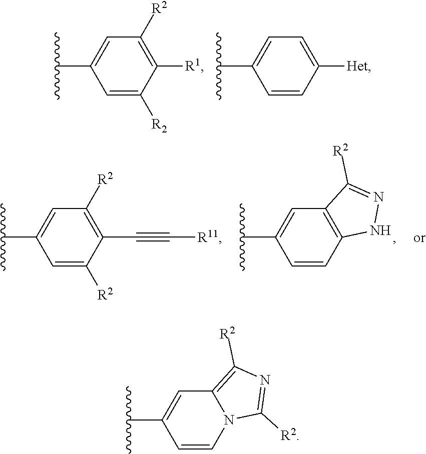

In some embodiments, Ar is

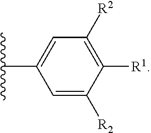

In some embodiments, R¹ is halogen, —CN or C₁-C₂ haloalkyl. In some embodiments, R¹ is —CN. In some embodiments, R² is H. In some embodiments, R²¹ is halogen, C₁-C₄ alkyl, C₃-C₅ cycloalkyl or 4- to 6-membered heterocycle. In some embodiments, R²¹ is C₁-C₄ alkyl, C₃-C₅ cycloalkyl or 4- to 6-membered heterocycle. In some embodiments, R²¹ is C₁-C₂ alkyl or C₃-C₅ cycloalkyl. In some embodiments, R²¹ is C₁-C₂ alkyl. In some embodiments, R²¹ is —CH₃. In some embodiments, R²² is H or C₁-C₂ alkyl. In some embodiments, R²² is H or —CH₃. In some embodiments, R²² is —CH₃. In some embodiments, each R²⁴ and R²⁵ is independently H, —CN, C₁-C₄ alkyl, —(C₁-C₄ alkyl)-OH, —(C₁-C₄ alkyl)-NR²⁴¹₂, —(C₁-C₄ alkyl)ₜ-Oᵤ—(C₃-C₅ cycloalkyl), —(C₁-C₄ alkyl)ₜ-Oᵤ-(4- to 6-membered heterocycle) or —(C₁-C₄ alkyl)-O—(C₁-C₄ alkyl). In some embodiments, each R²⁴ and R²⁵ is independently H, C₁-C₄ alkyl, —(C₁-C₄ alkyl)ₜ-Oᵤ-(4- to 6-membered heterocycle) or —(C₁-C₄ alkyl)-O—(C₁-C₄ alkyl). In some embodiments, R²⁴ is H, C₁-C₄ alkyl, —(C₁-C₄ alkyl)-OH, —(C₁-C₄ alkyl)-NR²⁴¹₂, —(C₁-C₄ alkyl)ₜ-Oᵤ—(C₃-C₅ cycloalkyl), —(C₁-C₄ alkyl)ₜ-Oᵤ-(4- to 6-membered heterocycle) or —(C₁-C₄ alkyl)-O—(C₁-C₄ alkyl). In some embodiments, R²⁴ is —CN, —Cl, C₁-C₄ alkyl or —(C₁-C₄ alkyl)-O—(C₁-C₄ alkyl). In some embodiments, R²⁴ is C₁-C₄ alkyl or —(C₁-C₄ alkyl)-O—(C₁-C₄ alkyl). In some embodiments, R²⁴ is —(C₁-C₂ alkyl)-O—(C₁-C₂ alkyl). In some embodiments, R²⁴ is C₁-C₄ alkyl. In some embodiments, R²⁴ is —CH₃. In some embodiments, R²⁴ is hydrogen. In some embodiments, R²⁴ is substituted with one or more substituents selected from halogen, C₃-C₅ cycloalkyl and C₁-C₂ alkoxy. In some embodiments, R²⁴ is substituted with one or more substituents selected from —F, cyclopropyl. In some embodiments, R²⁴ is substituted with two substituents that are the same or different. In some embodiments, R²⁴ is substituted with three substituents that are the same or different. In some embodiments, R²⁵ is halogen, methyl, ethyl or cyclopropyl. In some embodiments, R²⁵ is —CN, —Cl, C₁-C₄ alkyl, —(C₁-C₄ alkyl)ₜ-O—(C₃-C₅ cycloalkyl) or —(C₁-C₄ alkyl)ₜ-O—(C₁-C₄ alkyl). In some embodiments, R²⁵ is —CN, —Cl, —CH₃, —O—(C₃-C₅ cycloalkyl) or —O—(C₁-C₂ alkyl). In some embodiments, R²⁵ is —CN, —Cl, or C₁-C₄ alkyl. In some embodiments, R²⁵ is —CH₃. In some embodiments, R²⁵ is —Cl. In some embodiments, R²⁵ is substituted with one or more halogen. In some embodiments, R²⁵ is substituted with one or more —F. In some embodiments, R²⁵ is substituted by two substituents. In some embodiments, R²⁵ is substituted by three substituents. In some embodiments, R²¹ is-CH₃ and R²² is H or methyl. In some embodiments, R²¹ is C₁-C₂ alkyl or C₃-C₅ cycloalkyl and R²² is H or —CH₃. In some embodiments, R²¹ is-CH₃ and R²² is H. In some embodiments, R²⁴ is H or —CH₃ and R²⁵ is-C₁. In some embodiments, R¹ is-CN and R² is H. In some embodiments, R²¹ is C₁-C₂ alkyl or C₃-C₅ cycloalkyl and R²² is C₁-C₂ alkyl. In some embodiments, R²¹ is C₁-C₂ alkyl or C₃-C₅ cycloalkyl and R²² is H or C₁-C₂ alkyl. In some embodiments, R²¹ is C₁-C₂ alkyl and R²² is H or —CH₃. In some embodiments, R²¹ is C₁-C₂ alkyl and R²² is H.

In some embodiments, the FASN inhibitor is a compound of Formula (XIV-C):

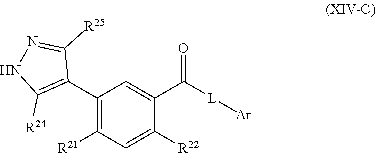

or a pharmaceutically acceptable salt thereof, wherein: L-Ar is

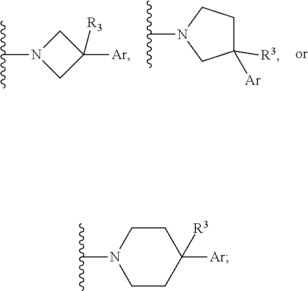

### Ar is

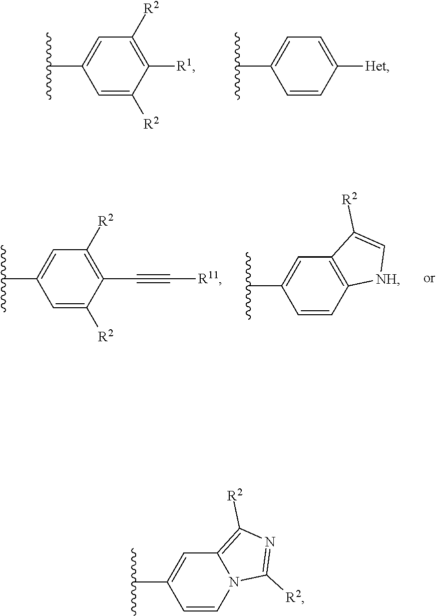

Het is a 5- to 6-membered heteroaryl; R¹ is H, —CN, halogen, C₁-C₄ alkyl, —O—(C₃-C₅ cycloalkyl), —O-(4- to 6-membered heterocycle) or —O—(C₁-C₄ alkyl), wherein when R¹ is not H, —CN or halogen, R¹ is optionally substituted with one or more halogens; each R² is independently hydrogen, halogen or C₁-C₄ alkyl; R³ is H or F; R¹¹ is H or —CH₃; R²¹ is H, halogen, C₁-C₄ alkyl, C₃-C₅ cycloalkyl or 4- to 6-membered heterocycle; R²² is H, halogen or C₁-C₂ alkyl; and each of R²⁴ and R²⁵ is independently H, —C₁-C₄ alkyl, or halogen.

As noted above, each of the C₁-C₂ alkyl, C₁-C₄ alkyl, C₃-C₅ cycloalkyl, 4- to 6-membered heterocycle and 5- to 6-membered heteroaryl moieties may be optionally substituted. Accordingly, the present disclosure provides for compounds of Formula (XIV-C) wherein: L-Ar is

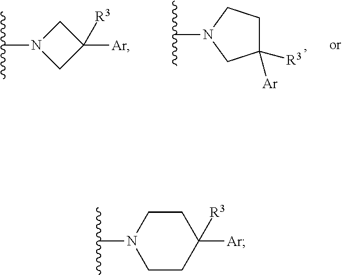

### Ar is

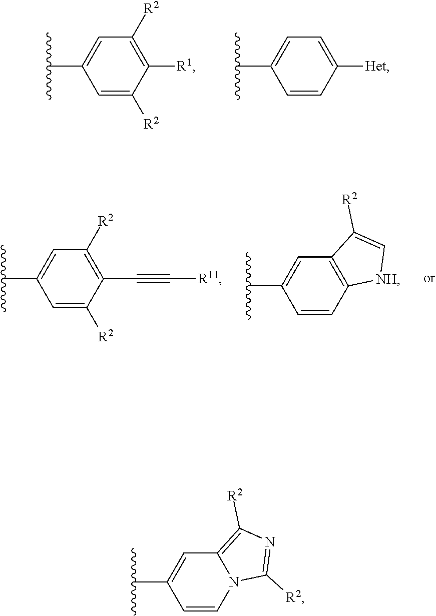

Het is an optionally substituted 5- to 6-membered heteroaryl; R¹ is H, —CN, halogen, optionally substituted C₁-C₄ alkyl, —O-(optionally substituted C₃-C₅ cycloalkyl), —O-(optionally substituted 4- to 6-membered heterocycle) or —O-(optionally substituted C₁-C₄ alkyl), wherein when R¹ is not H, —CN or halogen, R¹ is optionally substituted with one or more halogens; each R² is independently hydrogen, halogen or optionally substituted C₁-C₄ alkyl; R³ is H or F; R¹¹ is H or —CH₃; R²¹ is H, halogen, optionally substituted C₁-C₄ alkyl, optionally substituted C₃-C₅ cycloalkyl or optionally substituted 4- to 6-membered heterocycle; R²² is H, halogen or optionally substituted C₁-C₂ alkyl; and each of R²⁴ and R²⁵ is independently H, optionally substituted C₁-C₄ alkyl, or halogen.

In some embodiments, when L-Ar is

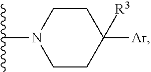

### Ar is not

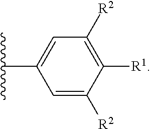

In some embodiments, L-Ar is

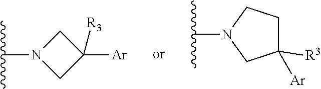

### and Ar is

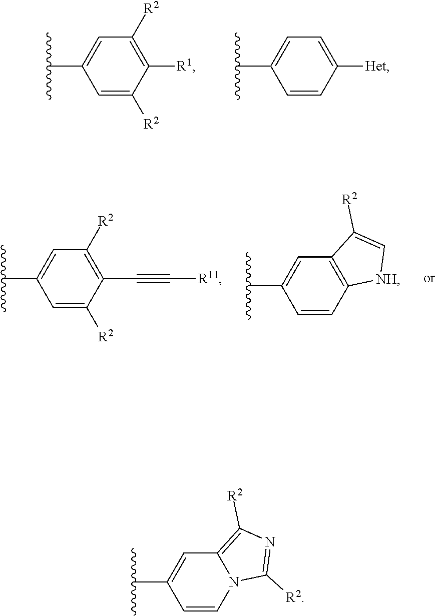

In some embodiments, L-Ar is

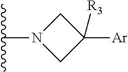

### and Ar is

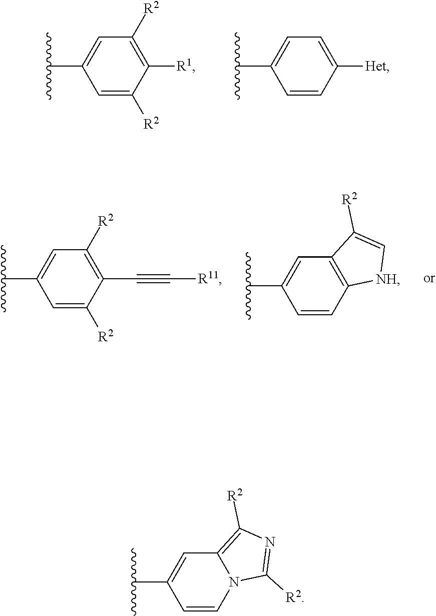

In some embodiments, R¹ is halogen, —CN or C₁-C₂ haloalkyl. In some embodiments, wherein R²¹ is halogen, C₁-C₄ alkyl, C₃-C₅ cycloalkyl or 4- to 6-membered heterocycle. In some embodiments, wherein R²¹ is —CH₃. In some embodiments, wherein R²² is H. In some embodiments, R²¹ is methyl, R²² is H, and L-Ar is

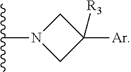

In some embodiments, the FASN inhibitor is a compound of Formula (XV):

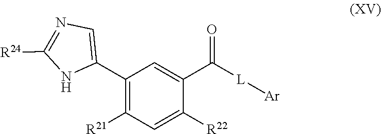

or pharmaceutically acceptable salt thereof, wherein: L-Ar is

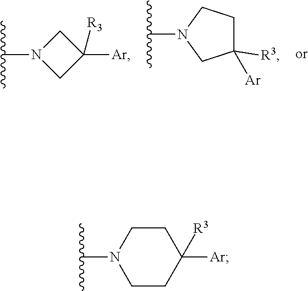

### Ar is

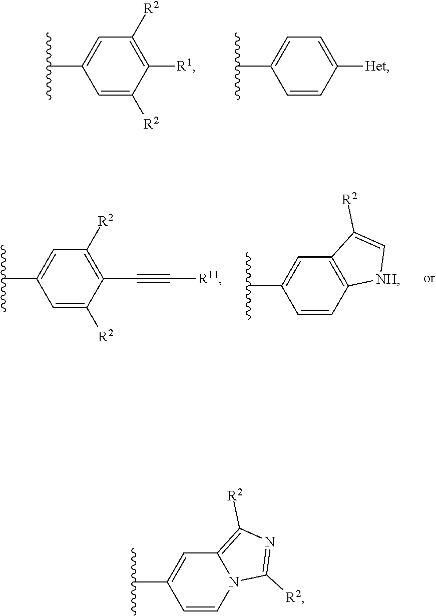

with the proviso that when L-Ar is

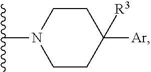

### Ar is not

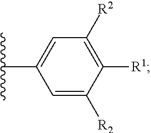

Het is a 5- to 6-membered heteroaryl; R¹ is H, —CN, halogen, C₁-C₄ alkyl, —O—(C₃-C₅ cycloalkyl), —O-(4- to 6-membered heterocycle) or —O—(C₁-C₄ alkyl), wherein when R¹ is not H, —CN or halogen, R¹ is optionally substituted with one or more halogens; each R² is independently hydrogen, halogen or C₁-C₄ alkyl; R³ is H or F; R¹¹ is H or —CH₃; R²¹ is H, halogen, C₁-C₄ alkyl, C₃-C₅ cycloalkyl or 4- to 6-membered heterocycle; R²² is H, halogen or C₁-C₂ alkyl; and R²⁴ is H, C₁-C₄ alkyl, —(C₁-C₄ alkyl)-OH, —(C₁-C₄ alkyl)-NR²⁴¹₂, —(C₁-C₄ alkyl)ₜ-Oᵤ—(C₃-C₅ cycloalkyl), —(C₁-C₄ alkyl)ₜ-Oᵤ-(4- to 6-membered heterocycle) or —(C₁-C₄ alkyl)-O—(C₁-C₄ alkyl), wherein: t is 0 or 1; u is 0 or 1; with the proviso that when u is 1, t is 1; and R²⁴¹ is H or C₁-C₂ alkyl.

As noted above, each of the C₁-C₂ alkyl, C₁-C₄ alkyl, C₃-C₅ cycloalkyl, 4- to 6-membered heterocycle and 5- to 6-membered heteroaryl moieties may be optionally substituted. Accordingly, the present disclosure provides for compounds of Formula (XV) wherein: L-Ar is

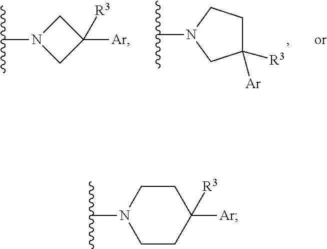

### Ar is

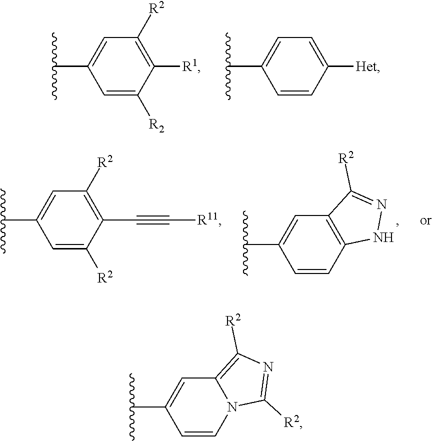

with the proviso that when L-Ar is

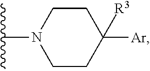

### Ar is not

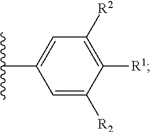

Het is an optionally substituted 5- to 6-membered heteroaryl; R¹ is H, —CN, halogen, optionally substituted C₁-C₄ alkyl, —O-(optionally substituted C₃-C₅ cycloalkyl), —O-(optionally substituted 4- to 6-membered heterocycle) or —O-(optionally substituted C₁-C₄ alkyl), wherein when R¹ is not H, —CN or halogen, R¹ is optionally substituted with one or more halogens; each R² is independently hydrogen, halogen or optionally substituted C₁-C₄ alkyl; R³ is H or F; R¹¹ is H or —CH₃; R²¹ is H, halogen, optionally substituted C₁-C₄ alkyl, optionally substituted C₃-C₅ cycloalkyl or optionally substituted 4- to 6-membered heterocycle; R²² is H, halogen or optionally substituted C₁-C₂ alkyl; R²⁴ is H, optionally substituted C₁-C₄ alkyl, -(optionally substituted C₁-C₄ alkyl) —OH, -(optionally substituted C₁-C₄ alkyl)-NR²⁴¹₂, -(optionally substituted C₁-C₄ alkyl)ₜ-Oᵤ-(optionally substituted C₃-C₅ cycloalkyl), -(optionally substituted C₁-C₄ alkyl)ₜ-Oᵤ-(optionally substituted 4- to 6-membered heterocycle) or -(optionally substituted C₁-C₄ alkyl)-O-(optionally substituted C₁-C₄ alkyl), wherein: t is 0 or 1; u is 0 or 1; with the proviso that when u is 1, t is 1; and R²⁴¹ is H or optionally substituted C₁-C₂ alkyl.

In some embodiments, L-Ar is

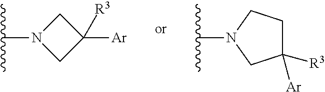

### and Ar is

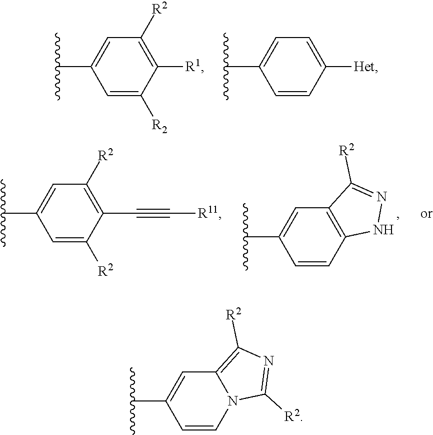

In some embodiments, R¹ is halogen, —CN or C₁-C₂ haloalkyl. In some embodiments, R¹ is —CN. In some embodiments, R² is H. In some embodiments, R²¹ is halogen, C₁-C₄ alkyl, C₃-C₅ cycloalkyl or 4- to 6-membered heterocycle. In some embodiments, R²¹ is C₁-C₂ alkyl or C₃-C₅ cycloalkyl. In some embodiments, R²¹ is C₁-C₂ alkyl. In some embodiments, R²¹ is C₃-C₅ cycloalkyl. In some embodiments, R²² is H or C₁-C₂ alkyl. In some embodiments, R²² is H. In some embodiments, R²² is C₁-C₂ alkyl. In some embodiments, R²² is —CH₃. In some embodiments, R²⁴ is C₁-C₄ alkyl or —(C₁-C₄ alkyl)-O—(C₁-C₄ alkyl). In some embodiments, R²⁴ is —(C₁-C₂ alkyl)-O—(C₁-C₂ alkyl). In some embodiments, R²¹ is C₁-C₂ alkyl or C₃-C₅ cycloalkyl and R²² is C₁-C₂ alkyl. In some embodiments, R²¹ is C₃-C₅ cycloalkyl and R²² is H or C₁-C₂ alkyl. In some embodiments, R²¹ is C₃-C₅ cycloalkyl and R²² is H or —CH₃. In some embodiments, R²¹ is C₃-C₅ cycloalkyl and R²² is H or —CH₃. In some embodiments, R¹ is —CN and R² is H.

In some embodiments, the FASN inhibitor is a compound of Formula (XVI):

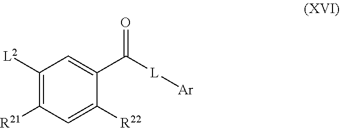

or a pharmaceutically acceptable salt thereof, wherein: L-Ar is

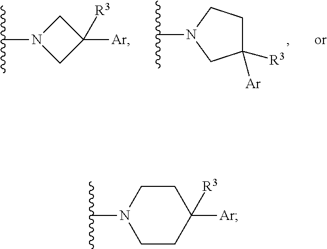

### Ar is

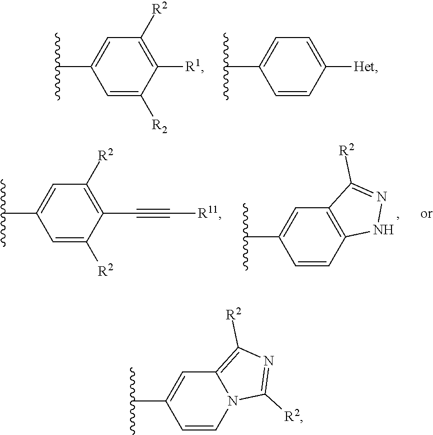

with the proviso that when L-Ar is

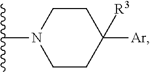

### Ar is not

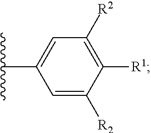

L² is —NHR³⁵ or —C(O)NHR³⁵¹, wherein R³⁵¹ is C₁-C₆ alkyl, C₃-C₅ cycloalkyl, 4- to 6-membered heterocycle, aryl or heteroaryl; Het is a 5- to 6-membered heteroaryl; R¹ is H, —CN, halogen, C₁-C₄ alkyl, —O—(C₃-C₅ cycloalkyl), —O-(4- to 6-membered heterocycle), —O—(C₁-C₄ alkyl) wherein when R¹ is not H, —CN or halogen, R¹ is optionally substituted with one or more halogens; each R² is independently hydrogen, halogen or C₁-C₄ alkyl; R³ is H or F; R¹¹ is H or —CH₃; R²¹ is H, halogen, C₁-C₄ alkyl, C₃-C₅ cycloalkyl or 4- to 6-membered heterocycle; R²² is H, halogen, or C₁-C₂ alkyl; and R³⁵ is —C(O)R³⁵¹, —C(O)NHR³⁵¹, C(O)OR³⁵¹ or S(O)₂R³⁵¹ wherein R³⁵¹ is C₁-C₆ alkyl, C₃-C₅ cycloalkyl, 4- to 6-membered heterocycle, aryl or heteroaryl.

As noted above, each of the C₁-C₂ alkyl, C₁-C₄ alkyl, C₁-C₆ alkyl, C₃-C₅ cycloalkyl, 4- to 6-membered heterocycle, 5- to 6-membered heteroaryl, aryl and heteroaryl moieties may be optionally substituted. Accordingly, the present disclosure provides for compounds of Formula (XVI) wherein:

L-Ar is

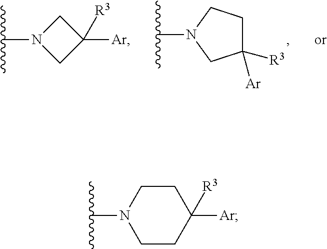

### Ar is

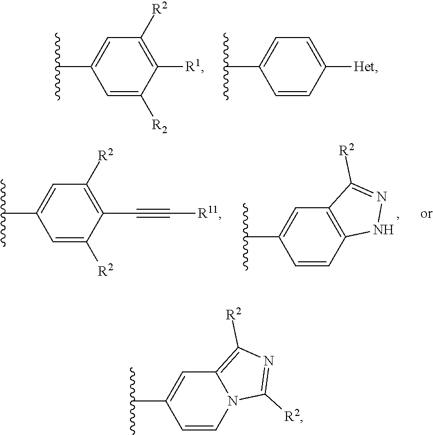

with the proviso that when L-Ar is

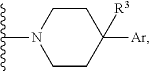

### Ar is not

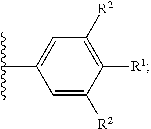

L² is —NHR³⁵ or —C(O)NHR³⁵¹, wherein R³⁵¹ is optionally substituted C₁-C₆ alkyl, optionally substituted C₃-C₅ cycloalkyl, optionally substituted 4- to 6-membered heterocycle, optionally substituted aryl or optionally substituted heteroaryl; Het is an optionally substituted 5- to 6-membered heteroaryl; R¹ is H, —CN, halogen, optionally substituted C₁-C₄ alkyl, —O-(optionally substituted C₃-C₅ cycloalkyl), —O-(optionally substituted 4- to 6-membered heterocycle) or —O-(optionally substituted C₁-C₄ alkyl), wherein when R¹ is not H, —CN or halogen, R¹ is optionally substituted with one or more halogens; each R² is independently hydrogen, halogen or optionally substituted C₁-C₄ alkyl; R³ is H or F; R¹¹ is H or —CH₃; R²¹ is H, halogen, optionally substituted C₁-C₄ alkyl, optionally substituted C₃-C₅ cycloalkyl or optionally substituted 4- to 6-membered heterocycle; R²² is H, halogen, or optionally substituted C₁-C₂ alkyl; and R³⁵ is —C(O)R³⁵¹, —C(O)NHR³⁵¹, —C(O)OR³⁵¹ or —S(O)₂R³⁵¹, wherein R³⁵¹ is optionally substituted C₁-C₆ alkyl, optionally substituted C₃-C₅ cycloalkyl, optionally substituted 4- to 6-membered heterocycle, optionally substituted aryl or optionally substituted heteroaryl.

In some embodiments, when L is

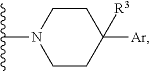

### Ar is not

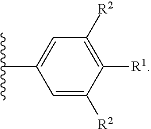

In some embodiments, the present disclosure provides for compounds of Structure V wherein L² is-NHR³⁵. In some embodiments, the present disclosure provides for compounds of Structure V wherein L² is-C(O)NHR³⁵¹

In some embodiments, the FASN inhibitor is a compound of Formula (XVII):

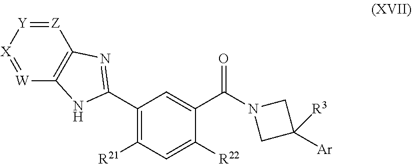

or a pharmaceutically acceptable salt thereof, wherein: each W, X, Y and Z is independently —N— or —CR²⁶— with the proviso that not more than 2 of W, X, Y and Z are —N—; each R²⁶ is independently H, C₁-C₄ alkyl, —O—(C₁-C₄ alkyl), —NR²⁷₂, —S(O)₂—(C₁-C₄ alkyl), or —C(O)—(C₁-C₄ alkyl); each R²⁷ is independently H or C₁-C₄ alkyl or both R²⁷ are C₁-C₄ alkyl and join to form a 3- to 6-membered ring together with the N to which they are attached and wherein the ring optionally includes one oxygen atom as one of the members of the ring; Ar is

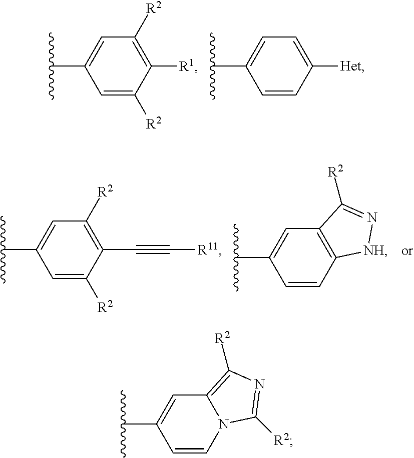

Het is a 5- to 6-membered heteroaryl; R¹ is H, —CN, halogen, C₁-C₄ alkyl, —O—(C₃-C₅s cycloalkyl), —O-(4- to 6-membered heterocycle), —O—(C₁-C₄ alkyl) wherein when R¹ is not H, —CN or halogen, R¹ is optionally substituted with one or more halogens; each R² is independently hydrogen, halogen or C₁-C₄ alkyl; R³ is H or F; R¹¹ is H or —CH₃; R²¹ is H, halogen, C₁-C₄ alkyl, C₃-C₅ cycloalkyl or a 4- to 6-membered heterocycle; and R²² is H, halogen or C₁-C₂ alkyl.

As noted above, each of the C₁-C₂ alkyl, C₁-C₄ alkyl, C₃-C₅ cycloalkyl, 4- to 6-membered heterocycle and 5- to 6-membered heteroaryl moieties may be optionally substituted. Accordingly, the present disclosure provides for compounds of Formula (XVII) wherein: each W, X, Y and Z is independently —N— or —CR²⁶— with the proviso that not more than 2 of W, X, Y and Z are —N—; R²⁶ is H, optionally substituted C₁-C₄ alkyl, —O-(optionally substituted C₁-C₄ alkyl), —NR²⁷₂, —S(O)₂-(optionally substituted C₁-C₄ alkyl) or —C(O)-(optionally substituted C₁-C₄ alkyl); each R²⁷ is independently H or optionally substituted C₁-C₄ alkyl or both R²⁷ are optionally substituted C₁-C₄ alkyl and join to form an optionally substituted 3- to 6-membered ring together with the N to which they are attached and wherein the ring optionally includes one oxygen atom as one of the members of the ring; Ar is

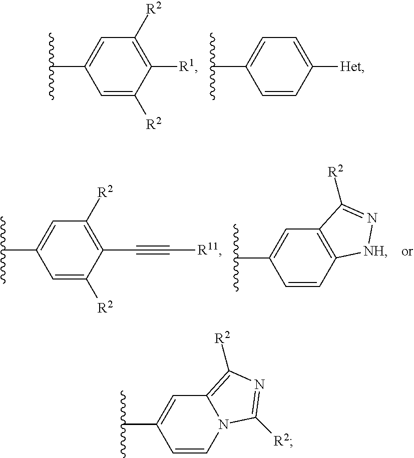

Het is an optionally substituted 5- to 6-membered heteroaryl; R¹ is H, —CN, halogen, optionally substituted C₁-C₄ alkyl, —O-(optionally substituted C₃-C₅ cycloalkyl), —O-(optionally substituted 4- to 6-membered heterocycle) or —O-(optionally substituted C₁-C₄ alkyl), wherein when R¹ is not H, —CN or halogen, R¹ is optionally substituted with one or more halogens; each R² is independently hydrogen, halogen or optionally substituted C₁-C₄ alkyl; R³ is H or F; R¹¹ is H or —CH₃; R²¹ is H, halogen, optionally substituted C₁-C₄ alkyl, optionally substituted C₃-C₅ cycloalkyl or an optionally substituted 4- to 6-membered heterocycle; and R²² is H, halogen or optionally substituted C₁-C₂ alkyl. In some embodiments, Ar is

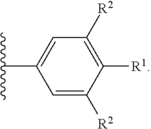

In some embodiments, Y is —CR²⁶— wherein R²⁶ is —NR²⁷₂. In some embodiments, X is —N—.

In some embodiments, the FASN inhibitor is a compound of Formula (XVIII):

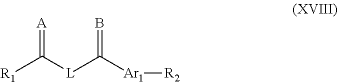

wherein: R₁ is a C₁-C₃ hydroxyl-alkyl either unsubstituted or substituted with —CH₃ or —CH_{z}F_{3-z}, 5 membered cycloalkyl either unsubstituted or substituted with substituents selected from the group consisting of deuterium, —Rₚ, —ORₚ, —NHR_{P}, and—NRₚRₚ₁; or 3 or 4 membered cycloalkyl or heterocycloalkyl wherein (i) the heteroatom ring member of the 3 or 4 membered heterocycloalkyl is independently selected from O, S, or N, and (ii) each of said 3 or 4 membered cycloalkyl or heterocycloalkyl is either unsubstituted or optionally substituted with substituents selected from the group consisting of deuterium, —Rₐ, —ORₐ, —NHRₐ, and —NRₐRₐ₁; L is a 5-10 membered monocyclic or bicyclic alkyl or heteroalkyl wherein (i) the heteroatom ring members of the 5-10 membered monocyclic or bicyclic heteroalkyl are independently selected from O, S, or N, and (ii) each of the 5-10 membered monocyclic or bicyclic alkyl or heteroalkyl is either unsubstituted or optionally substituted with substituents selected from the group consisting of deuterium and —R_{b}; A and B are independently O or S; Ar₁ is a 4-10 membered monocyclic or bicyclic aryl, heteroaryl or heterocycloalkyl, wherein (i) said 4-10 membered monocyclic or bicyclic heteroaryl or heterocycloalkyl have 1, 2, 3, or 4 heteroatoms which are independently selected from N, S or O, and (ii) each of said 4-10 membered monocyclic or bicyclic aryl, heteroaryl, or heterocycloalkyl is either unsubstituted or optionally independently substituted with 1 or more substituents which can be the same or different and are independently selected from the group consisting of deuterium, halo, alkyl, —CH_{z}F_{3-z}, cyano, hydroxyl, hydroxylalkyl, amino, aminoalkyl-, (amino)alkoxy-, —CONH₂, —C(O)NH(alkyl), —C(O)N(alkyl)₂, —C(O)NH(aryl), —C(O)N(aryl)₂, —OCH_{z}F_{3-z}, -alkyl, -alkenyl, -alkynyl, -alkoxy or (alkoxyalkyl)amino-, —NR_{c}—C(O)-alkyl, —NR_{c}—C(O)-aryl, -cycloalkyl, -heterocycloalkyl, -aryl, and -heteroaryl, with the proviso that no two adjacent ring heteroatoms are both S or both O; R² is H or a 4-15 membered monocyclic, bicyclic, or tricyclic aryl, heteroaryl, cycloalkyl, or heterocycloalkyl, (i) the 4-15 membered monocyclic, bicyclic, or tricyclic heteroaryl or heterocycloalkyl has 1, 2, 3, 4, 5, 6, 7, or 8 heteroatoms which are independently selected from N, S or O, and (ii) wherein each of said aryl, heteroaryl, cycloalkyl, and heterocycloalkyl is either unsubstituted or optionally substituted with 1 or more substituents which can be the same or different and are independently selected from the group consisting of deuterium, halo, cyano, hydroxyl, hydroxyl-alkyl-, hydroxylcycoalkyl-, hydroxyl-heterocycloalkyl-, hydroxyl-aryl-, hydroxyl-heteroaryl-, amino, aminoalkyl, (amino)alkoxy-, —CONH₂, —C(O)NH(alkyl), —C(O)N(alkyl)₂, —C(O)NH(aryl), —C(O)N(aryl)₂, —CH_{z}F_{3-z}, —OCH_{z}F_{3-z}, -alkyl, alkoxy-, -alkenyl, -alkynyl, aryloxy-, (alkoxyalkyl)amino-, -cycloalkyl, -heterocycloalkyl, (heterocycloalkyl)alkyl-, -aryl, -heteroaryl, —O(alkyl), —O(cycloalkyl), —O(heterocycloalkyl), —O(aryl), —O(heteroaryl), ONH₂, —C(O)NH(alkyl), —C(O)N(aryl)₂, —C(O)NH(cycloalkyl), —NH(CO)cycloalkyl, —NH(SO₂), —NH(SO₂)alkyl, —NH(SO₂)aryl, —NH(SO₂)heteroaryl, —N(SO₂)cycloalkyl, —C(O)N(alkyl)₂, (aryl)alkyl-, -heteroaryl, (heteroaryl)alkyl-, —S(O)₂-alkyl, —S(O)₂-aryl, —S(O)₂-cycloalkyl, —C(O)N(alkyl)₂, —C(O)alkyl, —NH—C(O)-alkyl, —NH—C(O)-cycloalkyl, NH—C(O)-heterocycloalkyl, NH—C(O)-heterocycloalkyl-Rd, —NH—C(O)-Rd-(O)alkyl, —NH—C(O)-aryl, —NH—C(O)—NH-alkyl, NH—C(O)—NH-cycloalkyl, NH₂(CO)cycloalkyl-, NH—C(O)—NH-aryl, —NH—C(O)—O-alkyl, NH—C(O)—NH-cycloalkyl, —NH—C(O)—O-cycloalkyl, —NH(R_{d})—C(O)-alkyl, —NH(R_{d})—C(O)-aryl, —NH(R_{d})—S(O₂)cycloalkyl, —S(O₂)NH₂, —S(O₂)NH(alkyl), —S(O₂)NR_{d}cycloalkyl, —S(O₂)N(alkyl)₂, —C(O)N(H)(alkyl), C(O)NR_{d}(cycloalkyl), methylenedioxy, —CH_{z}F_{3-z}, —OCH_{z}F_{3-z}, and -alkoxy; Rₚ and Rₚ₁ are independently H, halo, C₁-C₄ alkyl, or C₃-C₄ cycloalkyl; Rₐ and Rₐ₁ are independently H, halo, C₁-C₄ alkyl, or C₃-C₄ cycloalkyl; R_{b} is H, halo, C₁-C₄ alkyl, C₁-C₃ hydroxyl-alkyl, or C₃-C₄ cycloalkyl; R_{c} is H, halo, C₁-C₄ alkyl, or C₃-C₄ cycloalkyl; R_{d} is H, halo, C₁-C₄ alkyl, or C₃-C₄ cycloalkyl; and z is 0, 1 or 2; and pharmaceutically acceptable salts, solvates, esters, prodrugs and isomers thereof.

In some embodiments, the FASN inhibitor is a compound of Formula (XVIII-A):

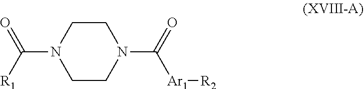

wherein: R₁ is a C₁-C₃ hydroxyl-alkyl either unsubstituted or substituted with —CH₃ or —CH_{z}F_{3-Z}, 5 membered cycloalkyl either unsubstituted or substituted with substituents selected from the group consisting of deuterium, —Rₚ, —ORₚ, —NHR_{P}, and —NRₚRₚ₁, or 3 or 4 membered cycloalkyl or heterocycloalkyl wherein (i) the heteroatom ring member of the 3 or 4 membered heterocycloalkyl is independently selected from O, S, or N, and (ii) each of said 3 or 4 membered cycloalkyl or heterocycloalkyl is either unsubstituted or optionally substituted with substituents selected from the group consisting of deuterium, —Rₐ, —ORₐ, —NHRₐ, and —NRₐRₐ₁; Ar₁ is a 4-10 membered monocyclic or bicyclic aryl, heteroaryl or heterocycloalkyl, wherein (i) said 4-10 membered monocyclic or bicyclic heteroaryl or heterocycloalkyl have 1, 2, 3, or 4 heteroatoms which are independently selected from N, S or O, and (ii) each of said 4-10 membered monocyclic or bicyclic aryl, heteroaryl, or heterocycloalkyl is either unsubstituted or optionally independently substituted with 1 or more substituents which can be the same or different and are independently selected from the group consisting of deuterium, halo, alkyl, —CH_{z}F_{3-z}, cyano, hydroxyl, hydroxylalkyl, amino, aminoalkyl-, (amino)alkoxy-, —CONH₂, —C(O)NH(alkyl), —C(O)N(alkyl)₂, —C(O)NH(aryl), —C(O)N(aryl)₂, —OCH_{z}F_{3-z}, -alkyl, -alkenyl, -alkynyl, -alkoxy or (alkoxyalkyl)amino-, —NR_{c}—C(O)-alkyl, —NR_{c}—C(O)-aryl, -cycloalkyl, -heterocycloalkyl, -aryl, and -heteroaryl, with the proviso that no two adjacent ring heteroatoms are both S or both O; R² is H or a 4-15 membered monocyclic, bicyclic, or tricyclic aryl, heteroaryl, cycloalkyl, or heterocycloalkyl, (i) the 4-15 membered monocyclic, bicyclic, or tricyclic heteroaryl or heterocycloalkyl has 1, 2, 3, 4, 5, 6, 7, or 8 heteroatoms which are independently selected from N, S or O, and (ii) wherein each of said aryl, heteroaryl, cycloalkyl, and heterocycloalkyl is either unsubstituted or optionally substituted with 1 or more substituents which can be the same or different and are independently selected from the group consisting of deuterium, halo, cyano, hydroxyl, hydroxyl-alkyl-, hydroxylcycoalkyl-, hydroxyl-heterocycloalkyl-, hydroxyl-aryl-, hydroxyl-heteroaryl-, amino, aminoalkyl, (amino)alkoxy-, —CONH₂, —C(O)NH(alkyl), —C(O)N(alkyl)₂, —C(O)NH(aryl), —C(O)N(aryl)₂, —CH_{z}F_{3-z}, —OCH_{z}F_{3-z}, -alkyl, alkoxy-, -alkenyl, -alkynyl, aryloxy-, (alkoxyalkyl)amino-, -cycloalkyl, -heterocycloalkyl, (heterocycloalkyl)alkyl-, -aryl, -heteroaryl, —O(alkyl), —O(cycloalkyl), —O(heterocycloalkyl), —O(aryl), —O(heteroaryl), ONH₂, —C(O)NH(alkyl), —C(O)N(aryl)₂, —C(O)NH(cycloalkyl), —NH(CO)cycloalkyl, —NH(SO₂), —NH(SO₂)alkyl, —NH(SO₂)aryl, —NH(SO₂)heteroaryl, —N(SO₂)cycloalkyl, —C(O)N(alkyl)₂, (aryl)alkyl-, -heteroaryl, (heteroaryl)alkyl-, —S(O)₂-alkyl, —S(O)₂-aryl, —S(O)₂-cycloalkyl, —C(O)N(alkyl)₂, —C(O)alkyl, —NH—C(O)-alkyl, —NH—C(O)-cycloalkyl, NH—C(O)-heterocycloalkyl, NH—C(O)-heterocycloalkyl-R_{d}, —NH—C(O)—R_{d}—(O)alkyl, —NH—C(O)-aryl, —NH—C(O)—NH-alkyl, NH—C(O)—NH-cycloalkyl, NH₂(CO)cycloalkyl-, NH—C(O)—NH-aryl, —NH—C(O)—O-alkyl, NH—C(O)—NH-cycloalkyl, —NH—C(O)—O-cycloalkyl, —NH(R_{d})—C(O)-alkyl, —NH(R_{d})—C(O)-aryl, —NH(R_{d})—S(O₂)cycloalkyl, —S(O₂)NH₂, —S(O₂)NH(alkyl), —S(O₂)NR_{d}cycloalkyl, —S(O₂)N(alkyl)₂, —C(O)N(H)(alkyl), —C(O)NR_{d}(cycloalkyl), methylenedioxy, —CH_{z}F_{3-Z}, —OCH_{z}F_{3-z}, and -alkoxy; Rₚ and Rₚ₁ are independently H, halo, C₁-C₄ alkyl, or C₃-C₄ cycloalkyl; Rₐ and Rₐ₁ are independently H, halo, C₁-C₄ alkyl, or C₃-C₄ cycloalkyl; Rc is H, halo, C₁-C₄ alkyl, or C₃-C₄ cycloalkyl; R_{d} is H, halo, C₁-C₄ alkyl, or C₃-C₄ cycloalkyl; and z is 0, 1 or 2; and pharmaceutically acceptable salts, solvates, esters, prodrugs and isomers thereof.

In some embodiments, the FASN inhibitor is a compound of Formula (XVIII-B):

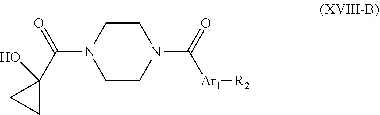

wherein: Ar₁ is a 4-10 membered monocyclic or bicyclic aryl, heteroaryl or heterocycloalkyl, wherein (i) said 4-10 membered monocyclic or bicyclic heteroaryl or heterocycloalkyl have 1, 2, 3, or 4 heteroatoms which are independently selected from N, S or O, and (ii) each of said 4-10 membered monocyclic or bicyclic aryl, heteroaryl, or heterocycloalkyl is either unsubstituted or optionally independently substituted with 1 or more substituents which can be the same or different and are independently selected from the group consisting of deuterium, halo, alkyl, —CH_{z}F_{3-z}, cyano, hydroxyl, hydroxylalkyl, amino, aminoalkyl-, (amino)alkoxy-, —CONH₂, —C(O)NH(alkyl), —C(O)N(alkyl)₂, —C(O)NH(aryl), —C(O)N(aryl)₂, —OCH_{z}F_{3-z}, -alkyl, -alkenyl, -alkynyl, -alkoxy or (alkoxyalkyl)amino-, —NR_{c}—C(O)-alkyl, —NR_{c}—C(O)-aryl, -cycloalkyl, -heterocycloalkyl, -aryl, and -heteroaryl, with the proviso that no two adjacent ring heteroatoms are both S or both O; R² is H or a 4-15 membered monocyclic, bicyclic, or tricyclic aryl, heteroaryl, cycloalkyl, or heterocycloalkyl, (i) the 4-15 membered monocyclic, bicyclic, or tricyclic heteroaryl or heterocycloalkyl has 1, 2, 3, 4, 5, 6, 7, or 8 heteroatoms which are independently selected from N, S or O, and (ii) wherein each of said aryl, heteroaryl, cycloalkyl, and heterocycloalkyl is either unsubstituted or optionally substituted with 1 or more substituents which can be the same or different and are independently selected from the group consisting of deuterium, halo, cyano, hydroxyl, hydroxyl-alkyl-, hydroxylcycoalkyl, hydroxyl-heterocycloalkyl-, hydroxyl-aryl-, hydroxyl-heteroaryl-, amino, aminoalkyl, (amino)alkoxy-, —CONH₂, —C(O)NH(alkyl), —C(O)N(alkyl)₂, —C(O)NH(aryl), —C(O)N(aryl)₂, —CH_{z}F_{3-z}, —OCH_{z}F_{3-z}, -alkyl, alkoxy-, -alkenyl, -alkynyl, aryloxy-, (alkoxyalkyl)amino-, -cycloalkyl, -heterocycloalkyl, (heterocycloalkyl)alkyl-, -aryl, -heteroaryl, —O(alkyl), —O(cycloalkyl), —O(heterocycloalkyl), —O(aryl), —O(heteroaryl), ONH₂, —C(O)NH(alkyl), —C(O)N(aryl)₂, —C(O)NH(cycloalkyl), —NH(CO)cycloalkyl, —NH(SO₂), —NH(SO₂)alkyl, —NH(SO₂)aryl, —NH(SO₂)heteroaryl, —N(SO₂)cycloalkyl, —C(O)N(alkyl)₂, (aryl)alkyl-, -heteroaryl, (heteroaryl)alkyl-, —S(O)₂-alkyl, —S(O)₂-aryl, —S(O)₂-cycloalkyl, —C(O)N(alkyl)₂, —C(O)alkyl, —NH—C(O)-alkyl, —NH—C(O)-cycloalkyl, NH—C(O)-heterocycloalkyl, NH—C(O)-heterocycloalkyl-R_{d}, —NH—C(O)—R_{d}—(O)alkyl, —NH—C(O)-aryl, —NH—C(O)—NH-alkyl, NH—C(O)—NH-cycloalkyl, NH₂(CO)cycloalkyl-, NH—C(O)—NH-aryl, —NH—C(O)—O-alkyl, NH—C(O)—NH-cycloalkyl, —NH—C(O)—O-cycloalkyl, —NH(R_{d})—C(O)-alkyl, —NH(R_{d})—C(O)-aryl, —NH(R_{d})—S(O₂)cycloalkyl, —S(O₂)NH₂, —S(O₂)NH(alkyl), —S(O₂)NR_{d}cycloalkyl, —S(O₂)N(alkyl)₂, —C(O)N(H)(alkyl), —C(O)NR_{d} (cycloalkyl), methylenedioxy, —CH_{z}F_{3-z}, —OCH_{z}F_{3-z}, and -alkoxy; Rc is H, halo, C₁-C₄ alkyl, or C₃-C₄ cycloalkyl; R_{d} is H, halo, C₁-C₄ alkyl, or C₃-C₄ cycloalkyl; and z is 0, 1 or 2; and pharmaceutically acceptable salts, solvates, esters, prodrugs and isomers thereof.

In some embodiments, the FASN inhibitor is a compound of Formula (XVIII-C):

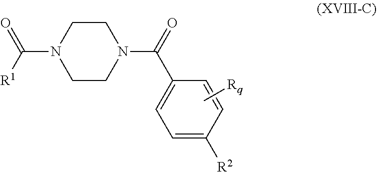

wherein: R₁ is a C₁-C₃ hydroxyl-alkyl either unsubstituted or substituted with —CH₃ or —CH_{z}F_{3-z}, 5 membered cycloalkyl either unsubstituted or substituted with substituents selected from the group consisting of deuterium, —Rₚ, —ORₚ, —NHRₚ, and —NRₚRₚ₁, or 3 or 4 membered cycloalkyl or heterocycloalkyl wherein (i) the heteroatom ring member of the 3 or 4 membered heterocycloalkyl is independently selected from O, S, or N, and (ii) each of said 3 or 4 membered cycloalkyl or heterocycloalkyl is either unsubstituted or optionally substituted with substituents selected from the group consisting of deuterium, —Rₐ, —ORₐ, —NHRₐ, and —NRₐRₐ₁; R₂ is H or a 4-15 membered monocyclic, bicyclic, or tricyclic aryl, heteroaryl, cycloalkyl, or heterocycloalkyl, (i) the 4-15 membered monocyclic, bicyclic, or tricyclic heteroaryl or heterocycloalkyl has 1, 2, 3, 4, 5, 6, 7, or 8 heteroatoms which are independently selected from N, S or O, and (ii) wherein each of said aryl, heteroaryl, cycloalkyl, and heterocycloalkyl is either unsubstituted or optionally substituted with 1 or more substituents which can be the same or different and are independently selected from the group consisting of deuterium, halo, cyano, hydroxyl, hydroxyl-alkyl-, hydroxylcycoalkyl-, hydroxyl-heterocycloalkyl-, hydroxyl-aryl-, hydroxyl-heteroaryl-, amino, aminoalkyl, (amino)alkoxy-, —CONH₂, —C(O)NH(alkyl), —C(O)N(alkyl)₂, —C(O)NH(aryl), —C(O)N(aryl)₂, —CH_{z}F_{3-z}, —OCH_{z}F_{3-z}, -alkyl, alkoxy-, -alkenyl, -alkynyl, aryloxy-, (alkoxyalkyl)amino-, -cycloalkyl, -heterocycloalkyl, (heterocycloalkyl)alkyl-, -aryl, -heteroaryl, —O(alkyl), —O(cycloalkyl), O(heterocycloalkyl), —O(aryl), —O(heteroaryl), ONH₂, —C(O)NH(alkyl), —C(O)N(aryl)₂, —C(O)NH(cycloalkyl), —NH(CO)cycloalkyl, —NH(SO₂), —NH(SO₂)alkyl, —NH(SO₂)aryl, —NH(SO₂)heteroaryl, —N(SO₂)cycloalkyl, —C(O)N(alkyl)₂, (aryl)alkyl-, -heteroaryl, (heteroaryl)alkyl-, —S(O)₂-alkyl, —S(O)₂-aryl, —S(O)₂-cycloalkyl, —C(O)N(alkyl)₂, —C(O)alkyl, —NH—C(O)-alkyl, —NH—C(O)-cycloalkyl, NH—C(O)-heterocycloalkyl, NH—C(O)-heterocycloalkyl-R_{d}, —NH—C(O)—R_{d}—(O)alkyl, —NH—C(O)-aryl, —NH—C(O)—NH-alkyl, NH—C(O)—NH-cycloalkyl, NH₂(CO)cycloalkyl-, NH—C(O)—NH-aryl, —NH—C(O)—O-alkyl, NH—C(O)—NH-cycloalkyl, —NH—C(O)—O-cycloalkyl, —NH(R_{d})—C(O)-alkyl, —NH(R_{d})—C(O)-aryl, —NH(R_{d})—S(O₂)cycloalkyl, —S(O₂)NH₂, —S(O₂)NH(alkyl), —S(O₂)NR_{d}cycloalkyl, —S(O₂)N(alkyl)₂, —C(O)N(H)(alkyl), —C(O)NR_{d}(cycloalkyl), methylenedioxy, —CH_{z}F_{3-z}, —OCH_{z}F_{3-z}, and alkoxy; Rₚ and Rₚ₁ are independently H, halo, C₁-C₄ alkyl, or C₃-C₄ cycloalkyl; Rₐ and Rₐ₁ are independently H, halo, C₁-C₄ alkyl, or C₃-C₄ cycloalkyl; R_{d} is H, halo, C₁-C₄ alkyl, or C₃-C₄ cycloalkyl; R_{q} is H, halo, C₁-C₄ alkyl, or C₃-C₄ cycloalkyl; and z is 0, 1 or 2; and pharmaceutically acceptable salts, solvates, esters, prodrugs and isomers thereof.

In some embodiments, the FASN inhibitor is a compound of Formula (XVIII-D):

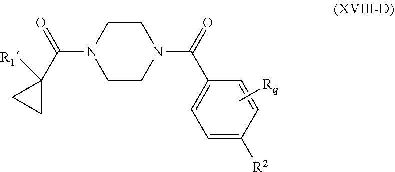

wherein: R_{1′} is OH or NH₂; R₂ is H or a 4-15 membered monocyclic, bicyclic, or tricyclic aryl, heteroaryl, cycloalkyl, or heterocycloalkyl, (i) the 4-15 membered monocyclic, bicyclic, or tricyclic heteroaryl or heterocycloalkyl has 1, 2, 3, 4, 5, 6, 7, or 8 heteroatoms which are independently selected from N, S or O, and (ii) wherein each of said aryl, heteroaryl, cycloalkyl, and heterocycloalkyl is either unsubstituted or optionally substituted with 1 or more substituents which can be the same or different and are independently selected from the group consisting of deuterium, halo, cyano, hydroxyl, hydroxyl-alkyl-, hydroxylcycoalkyl-, hydroxyl-heterocycloalkyl-, hydroxyl-aryl-, hydroxyl-heteroaryl-, amino, aminoalkyl, (amino)alkoxy-, —CONH₂, —C(O)NH(alkyl), —C(O)N(alkyl)₂, —C(O)NH(aryl), —C(O)N(aryl)₂, —CH_{z}F_{3-z}, —OCH_{z}F_{3-z}, -alkyl, alkoxy-, -alkenyl, -alkynyl, aryloxy-, (alkoxyalkyl)amino-, -cycloalkyl, -heterocycloalkyl, (heterocycloalkyl)alkyl-, -aryl, -heteroaryl, —O(alkyl), —O(cycloalkyl), —O(heterocycloalkyl), —O(aryl), —O(heteroaryl), ONH₂, —C(O)NH(alkyl), —C(O)N(aryl)₂, —C(O)NH(cycloalkyl), —NH(CO)cycloalkyl, —NH(SO₂), —NH(SO₂)alkyl, —NH(SO₂)aryl, —NH(SO₂)heteroaryl, —N(SO₂)cycloalkyl, —C(O)N(alkyl)₂, (aryl)alkyl-, -heteroaryl, (heteroaryl)alkyl-, —S(O)₂-alkyl, —S(O)₂-aryl, —S(O)₂-cycloalkyl, —C(O)N(alkyl)₂, —C(O)alkyl, —NH—C(O)-alkyl, —NH—C(O)-cycloalkyl, NH—C(O)-heterocycloalkyl, NH—C(O)-heterocycloalkyl-R_{d}, —NH—C(O)—R_{d}—(O)alkyl, —NH—C(O)-aryl, —NH—C(O)—NH-alkyl, NH—C(O)—NH-cycloalkyl, NH₂(CO)cycloalkyl-, NH—C(O)—NH-aryl, —NH—C(O)—O-alkyl, NH—C(O)—NH-cycloalkyl, —NH—C(O)—O-cycloalkyl, —NH(R_{d})—C(O)-alkyl, —NH(R_{d})—C(O)-aryl, —NH(R_{d})—S(O₂)cycloalkyl, —S(O₂)NH₂, —S(O₂)NH(alkyl), —S(O₂)NR_{d}cycloalkyl, —S(O₂)N(alkyl)₂, —C(O)N(H)(alkyl), —C(O)NR_{d}(cycloalkyl), methylenedioxy, —CH_{z}F_{3-z}, —OCH_{z}F_{3-z}, and -alkoxy; R_{d} is H, halo, C₁-C₄ alkyl, or C₃-C₄ cycloalkyl; and z is 0, 1 or 2; and pharmaceutically acceptable salts, solvates, esters, prodrugs and isomers thereof.

In the compounds of Formulas (XVIII), (XVIII-A), (XVIII-B), (XVIII-C), and (XVIII-D), the various moieties are independently selected.

The following embodiments are directed to Formulas (XVIII), (XVIII-A), (XVIII-B), (XVIII-C), and (XVIII-D), as applicable. For any moieties that are not specifically defined, the previous definitions control. Further, the moieties aryl, heteroaryl, and heterocycloalkyl in these embodiments can be independently unsubstituted or optionally substituted or optionally fused as described earlier.

In some embodiments, R₁ is C₁-C₃ hydroxyl-alkyl either unsubstituted or substituted with —CH₃ or —CH_{z}F_{3-z}. In some embodiments, R₁ is a 5 membered cycloalkyl either unsubstituted or substituted with hydroxyl. In some embodiments, R₁ is a 3 or 4 membered cycloalkyl. In some embodiments, R₁ is a 3 or 4 membered heterocycloalkyl. In some embodiments, R₁ is

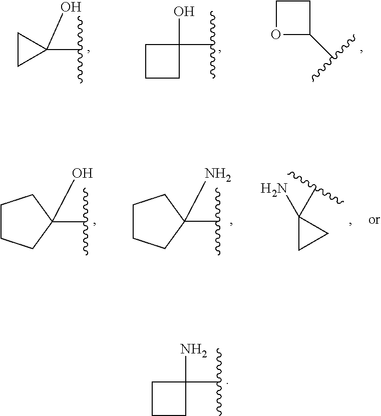

In some embodiments, R₁ is

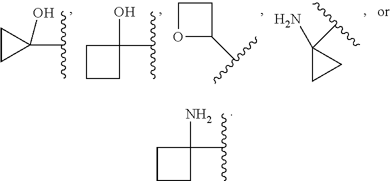

In some embodiments, R₁ is

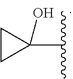 [SMILES: *C1(O)CC1]

In some embodiments, R₁ is

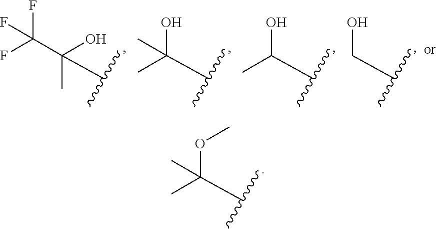

In some embodiments, A and B are O. In some embodiments, A and B are S. In some embodiments, either A or B is 0, and the other is S. In some embodiments, L is a 5-10 membered monocyclic alkyl. In some embodiments, L is a 5-10 membered bicyclic alkyl. In some embodiments, L is a 5-10 membered monocyclic heteroalkyl. In some embodiments, L is a 5-10 membered bicyclic heteroalkyl.

In some embodiments, L is

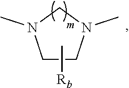

wherein m is 1, 2, or 3 and n is 0, 1, 2, or 3. In some embodiments, L is

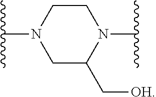 [SMILES: *N1CCN(*)C(CO)C1]

In some embodiments, L is

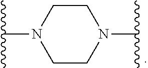 [SMILES: *N1CCN(*)CC1]

In some embodiments, Ar₁ is an aryl. In some embodiments, Ar₁ is a heteroaryl. In some embodiments, Ar₁ is a 5-10 membered monocyclic aryl. In some embodiments, Ar₁ is a 5-10 membered bicyclic aryl. In some embodiments, Ar₁ is a 5-10 membered monocyclic heteroaryl. In some embodiments, Ar₁ is a 5-10 membered bicyclic heteroaryl. In some embodiments, Ar₁ is an optionally substituted 5 membered monocyclic aryl or heteroaryl, said heteroaryl having 1 or 2 heteroatoms selected, independently, from S or N. In some embodiments, Ar₁ is an optionally substituted form of

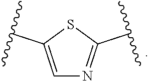 [SMILES: *c1cnc(*)s1]

In some embodiments, Ar₁ is an optionally substituted 6 membered monocyclic aryl or heteroaryl, said heteroaryl having 1 or 2 heteroatoms which are N. In some embodiments, Ar₁ is an optionally substituted form of

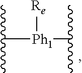

wherein Ph₁ is phenyl, pyridinyl, pyrazinyl, or pyrimidinyl, and Rₑ is H, halo, or C₁-C₃ alkyl.

In some embodiments, Ar₁ is an optionally substituted form of

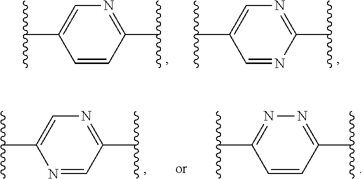

In some embodiments, Ar₁ is an optionally substituted 6 membered monocyclic aryl. In some embodiments, Ar₁ is

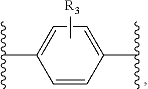

wherein Rₑ is H, halo, or C₁-C₃ alkyl. In some embodiments, Ar₁ is

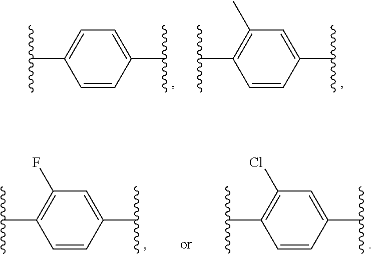

In some embodiments, Ar₁ is an optionally substituted 9 membered 6,5-bicyclic heteroaryl, said heteroaryl having 1, 2, or 3 heteroatoms independently selected from O, S, and N. In some embodiments, Ar₁ is an optionally substituted form of

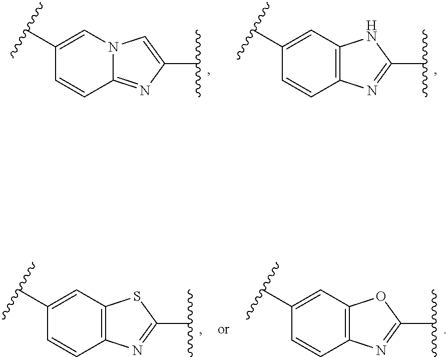 [SMILES: *c1ccc2nc(*)cn2c1 ; *c1ccc2nc(*)[nH]c2c1 ; *c1ccc2nc(*)sc2c1 ; *c1ccc2nc(*)oc2c1]

In some embodiments, R₂ is an optionally substituted aryl. In some embodiments, R₂ is an optionally substituted heteroaryl. In some embodiments, R₂ is an optionally substituted cycloalkyl. In some embodiments, R₂ is an optionally substituted heterocycloalkyl. In some embodiments, R₂ is an optionally substituted monocyclic or bicyclic 5-10 membered aryl or heteroaryl. In some embodiments, R₂ is an optionally substituted monocyclic 6 membered aryl. In some embodiments, R₂ is

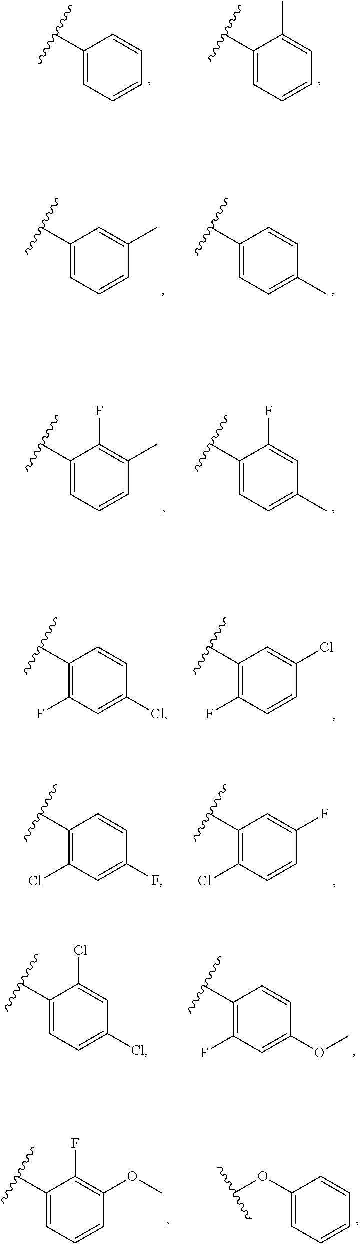

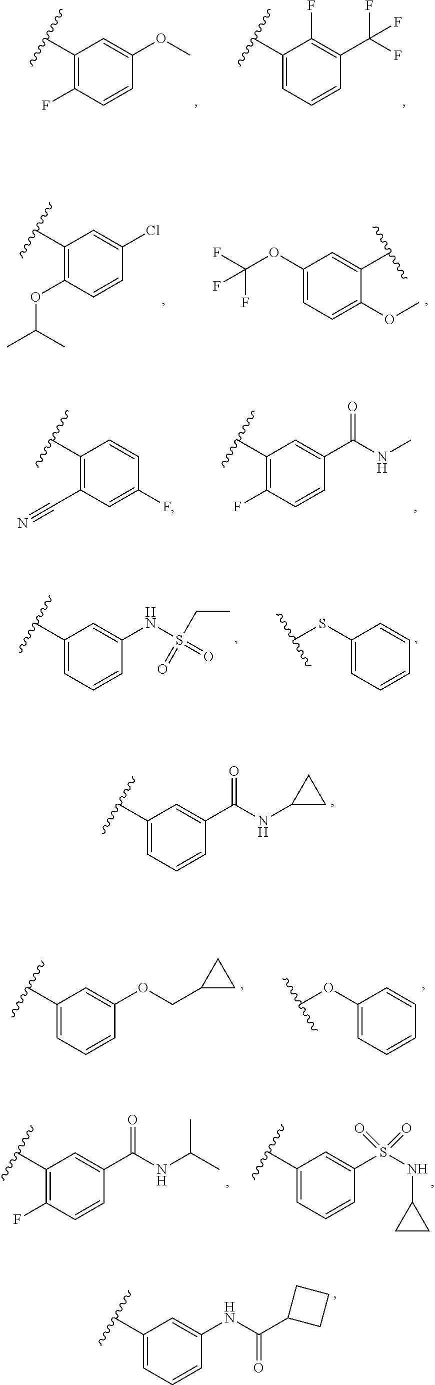

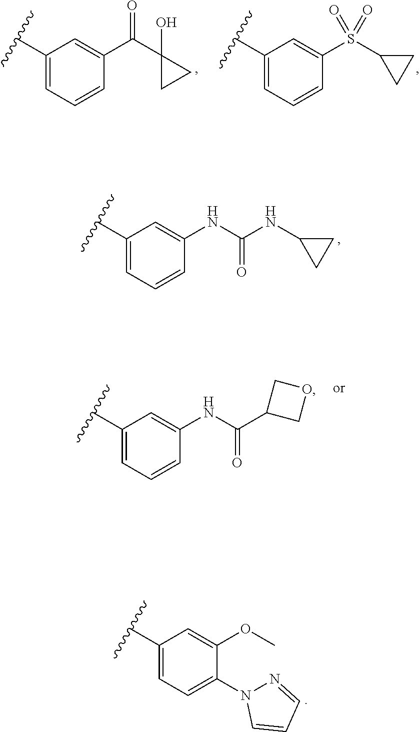 [SMILES: *c1cccc(C(=O)C2(O)CC2)c1 ; *c1cccc(S(=O)(=O)C2CC2)c1 ; *c1cccc(NC(=O)NC2CC2)c1 ; *c1cccc(NC(=O)C2COC2)c1 ; *c1ccc(-n2cccn2)c(OC)c1]

In some embodiments, R₂ is an optionally substituted bicyclic 8-10 membered aryl or 8-10 membered heteroaryl. In some embodiments, R₂ is an optionally substituted 8 membered 5,5 bicyclic heteroaryl, said heteroaryl having 1, 2, 3, or 4 heteroatoms, wherein said heteroatoms are independently selected from O, S, and N. In some embodiments, R₂ is an optionally substituted form of

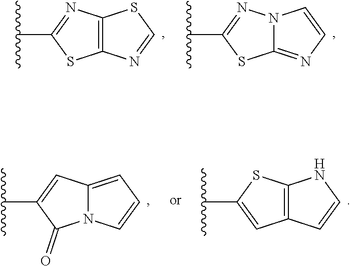

In some embodiments, R₂ is an optionally substituted 9 membered 6,5 bicyclic heteroaryl, said heteroaryl having 1, 2, 3, or 4 heteroatoms, wherein said heteroatoms are independently selected from O, S, and N. In some embodiments, R₂ is an optionally substituted form of

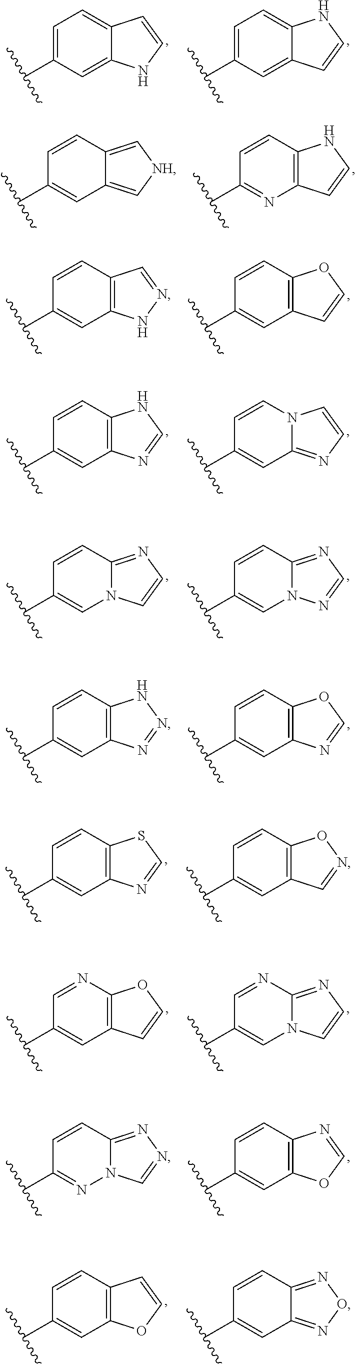

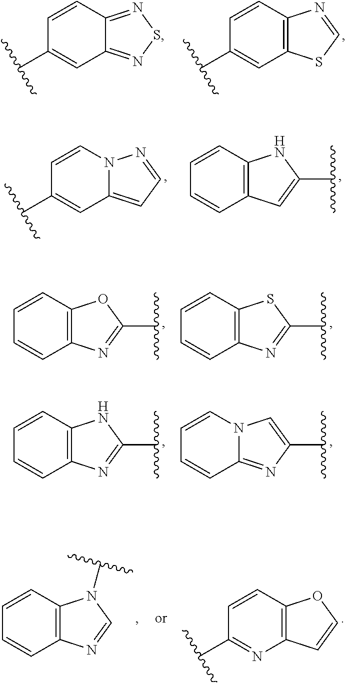

In some embodiments, R₂ is an optionally substituted 10 membered 6,6 bicyclic aryl or heteroaryl, said heteroaryl having 1, 2, 3, or 4 heteroatoms, wherein said heteroatoms are selected from O, S, and N. In some embodiments, R₂ is an optionally substituted form of

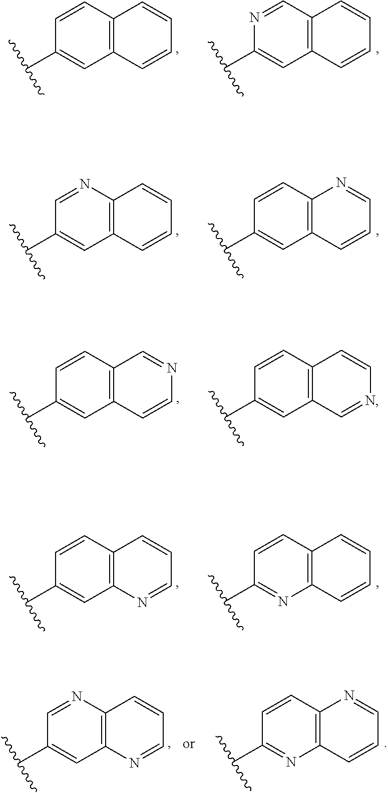

In some embodiments, Rₚ is H. In some embodiments, Rₚ is halo. In some embodiments, Rₚ is C₁-C₄ alkyl. In some embodiments, Rₚ is C₃-C₄ cycloalkyl. In some embodiments, Rₚ₁ is H. In some embodiments, Rₚ₁ is halo. In some embodiments, Rₚ₁ is C₁-C₄ alkyl. In some embodiments, Rₚ₁ is C₃-C₄ cycloalkyl. In some embodiments, Rₐ is H. In some embodiments, Rₐ is halo. In some embodiments, Rₐ is C₁-C₄ alkyl. In some embodiments, Rₐ is C₃-C₄ cycloalkyl. In some embodiments, Rₐ₁ is H. In some embodiments, Rₐ₁ is halo. In some embodiments, Rₐ₁ is C₁-C₄ alkyl. In some embodiments, Rₐ₁ is C₃-C₄ cycloalkyl. In some embodiments, R_{b} is H. In some embodiments, R_{b} is halo. In some embodiments, R_{b} is C₁-C₄ alkyl. In some embodiments, R_{b} is C₁-C₃ hydroxyl-alkyl. In some embodiments, R_{b} is C₃-C₄ cycloalkyl. In some embodiments, R_{c} is H. In some embodiments, R_{c} is halo. In some embodiments, R_{c} is C₁-C₄ alkyl. In some embodiments, R_{c} is C₃-C₄ cycloalkyl. In some embodiments, R_{d} is H. In some embodiments, R_{d} is halo. In some embodiments, R_{d} is C₁-C₄ alkyl. In some embodiments, R_{d} is C₃-C₄ cycloalkyl. In some embodiments, R_{q} is H. In some embodiments, R_{q} is halo. In some embodiments, R_{q} is C₁-C₄ alkyl. In some embodiments, R_{q} is C₃-C₄ cycloalkyl. In some embodiments, z is 0. In some embodiments, z is 1. In some embodiments, z is 2. In some embodiments, R² is not an optionally substituted form of

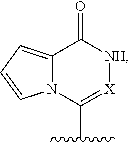

wherein X is N or CH. In some embodiments, Ar₁ is

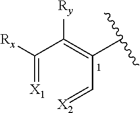

connected to

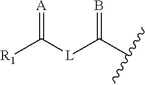

at position 1, and each of X₁ and X₂ is, independently, N or C—R_{z}, and R_{y} and R_{z} are any substituent, then Rₓ does not include alkynyl, alkenyl, aryl, 5-14 membered heterocycle, 5-14 membered heteroaryl, or 4-9 membered carbocycle. In some embodiments, when R₂ is

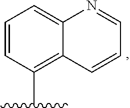 [SMILES: *c1cccc2ncccc12]

Ar₁ is not an optionally substituted form of

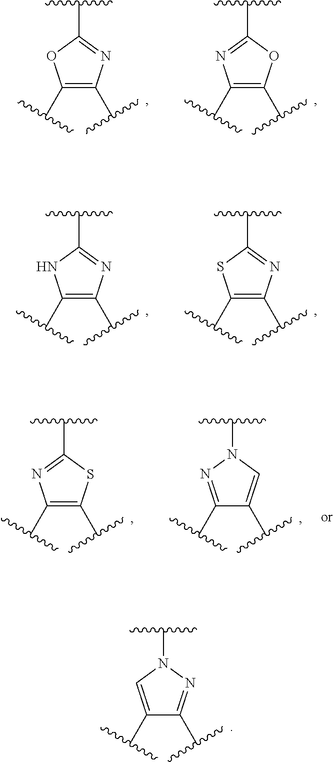

In some embodiments, Ar₁ is

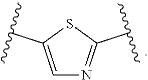 [SMILES: *c1cnc(*)s1]

In some embodiments, the compound is one of the following:

| | Compound |
|---|---|
| 1 | 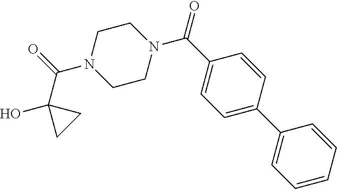 [SMILES: O=C(c1ccc(-c2ccccc2)cc1)N1CCN(C(=O)C2(O)CC2)CC1] |
| 2 | 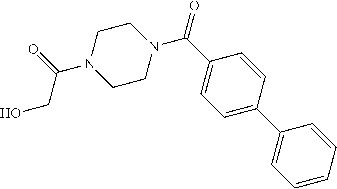 [SMILES: O=C(CO)N1CCN(C(=O)c2ccc(-c3ccccc3)cc2)CC1] |
| 3 | 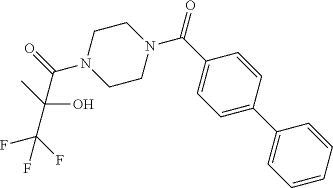 [SMILES: CC(O)(C(=O)N1CCN(C(=O)c2ccc(-c3ccccc3)cc2)CC1)C(F)(F)F] |
| 4 | 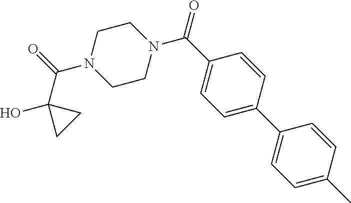 [SMILES: Cc1ccc(-c2ccc(C(=O)N3CCN(C(=O)C4(O)CC4)CC3)cc2)cc1] |
| 5 | 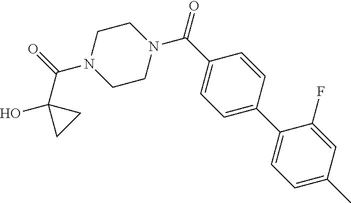 [SMILES: Cc1ccc(-c2ccc(C(=O)N3CCN(C(=O)C4(O)CC4)CC3)cc2)c(F)c1] |
| 6 | 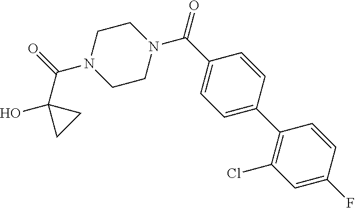 [SMILES: O=C(c1ccc(-c2ccc(F)cc2Cl)cc1)N1CCN(C(=O)C2(O)CC2)CC1] |
| 7 | 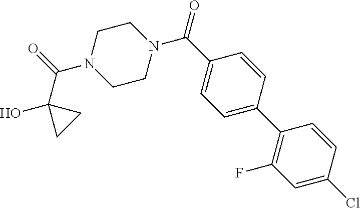 [SMILES: O=C(c1ccc(-c2ccc(Cl)cc2F)cc1)N1CCN(C(=O)C2(O)CC2)CC1] |
| 8 | 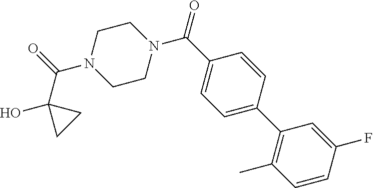 [SMILES: Cc1ccc(F)cc1-c1ccc(C(=O)N2CCN(C(=O)C3(O)CC3)CC2)cc1] |
| 9 | 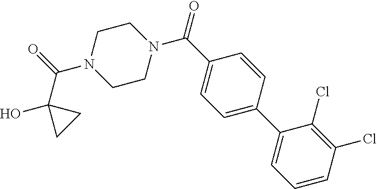 [SMILES: O=C(c1ccc(-c2cccc(Cl)c2Cl)cc1)N1CCN(C(=O)C2(O)CC2)CC1] |
| 10 | 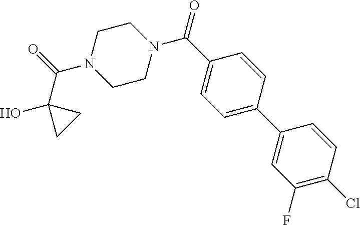 [SMILES: O=C(c1ccc(-c2ccc(Cl)c(F)c2)cc1)N1CCN(C(=O)C2(O)CC2)CC1] |
| 11 | 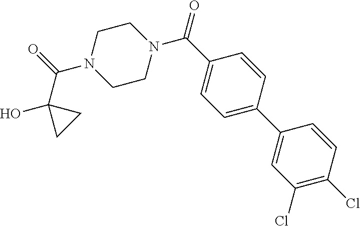 [SMILES: O=C(c1ccc(-c2ccc(Cl)c(Cl)c2)cc1)N1CCN(C(=O)C2(O)CC2)CC1] |
| 12 | 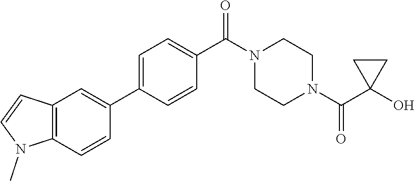 [SMILES: Cn1ccc2cc(-c3ccc(C(=O)N4CCN(C(=O)C5(O)CC5)CC4)cc3)ccc21] |
| 13 | 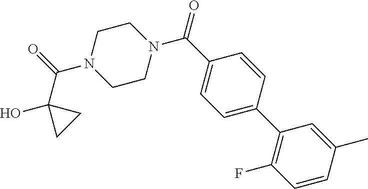 [SMILES: Cc1ccc(F)c(-c2ccc(C(=O)N3CCN(C(=O)C4(O)CC4)CC3)cc2)c1] |
| 14 | 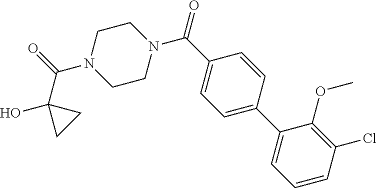 [SMILES: COc1c(Cl)cccc1-c1ccc(C(=O)N2CCN(C(=O)C3(O)CC3)CC2)cc1] |
| 15 | 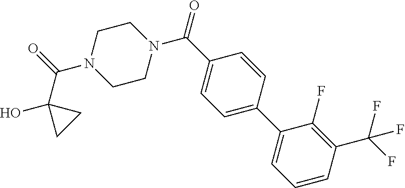 [SMILES: O=C(c1ccc(-c2cccc(C(F)(F)F)c2F)cc1)N1CCN(C(=O)C2(O)CC2)CC1] |
| 16 | 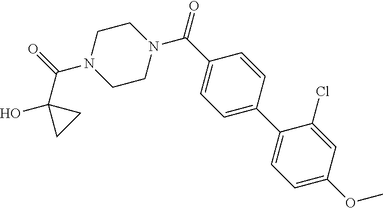 [SMILES: COc1ccc(-c2ccc(C(=O)N3CCN(C(=O)C4(O)CC4)CC3)cc2)c(Cl)c1] |
| 17 | 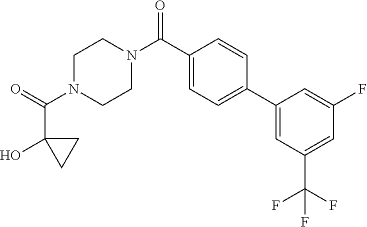 [SMILES: O=C(c1ccc(-c2cc(F)cc(C(F)(F)F)c2)cc1)N1CCN(C(=O)C2(O)CC2)CC1] |
| 18 | 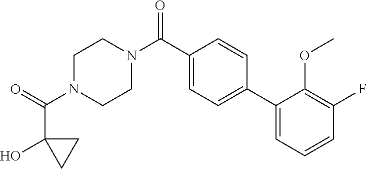 [SMILES: COc1c(F)cccc1-c1ccc(C(=O)N2CCN(C(=O)C3(O)CC3)CC2)cc1] |
| 19 | 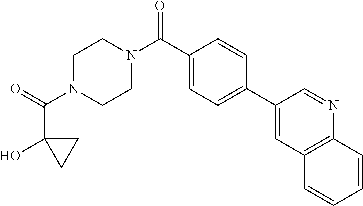 [SMILES: O=C(c1ccc(-c2cnc3ccccc3c2)cc1)N1CCN(C(=O)C2(O)CC2)CC1] |
| 20 | 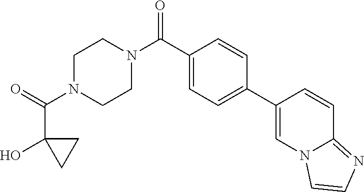 [SMILES: O=C(c1ccc(-c2ccc3nccn3c2)cc1)N1CCN(C(=O)C2(O)CC2)CC1] |
| 21 | 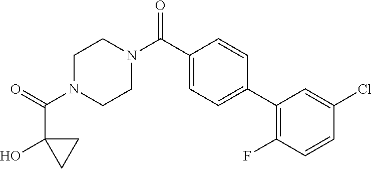 [SMILES: O=C(c1ccc(-c2cc(Cl)ccc2F)cc1)N1CCN(C(=O)C2(O)CC2)CC1] |
| 22 | 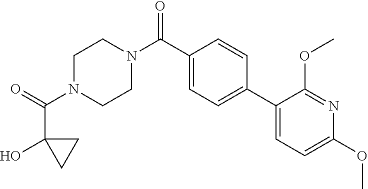 [SMILES: COc1ccc(-c2ccc(C(=O)N3CCN(C(=O)C4(O)CC4)CC3)cc2)c(OC)n1] |
| 23 | 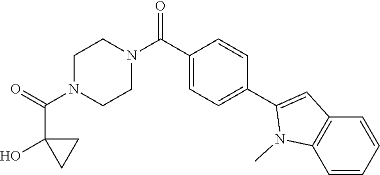 [SMILES: Cn1c(-c2ccc(C(=O)N3CCN(C(=O)C4(O)CC4)CC3)cc2)cc2ccccc21] |
| 24 | 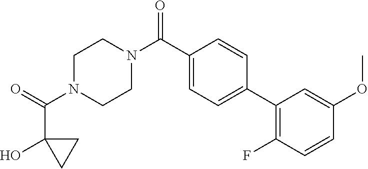 [SMILES: COc1ccc(F)c(-c2ccc(C(=O)N3CCN(C(=O)C4(O)CC4)CC3)cc2)c1] |
| 25 | 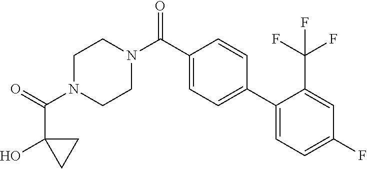 [SMILES: O=C(c1ccc(-c2ccc(F)cc2C(F)(F)F)cc1)N1CCN(C(=O)C2(O)CC2)CC1] |
| 26 | 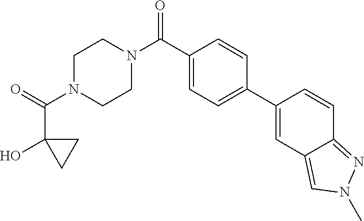 [SMILES: Cn1cc2cc(-c3ccc(C(=O)N4CCN(C(=O)C5(O)CC5)CC4)cc3)ccc2n1] |
| 27 | 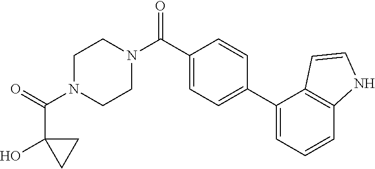 [SMILES: O=C(c1ccc(-c2cccc3[nH]ccc23)cc1)N1CCN(C(=O)C2(O)CC2)CC1] |
| 28 | 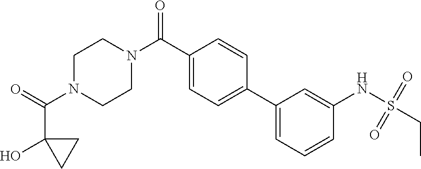 [SMILES: CCS(=O)(=O)Nc1cccc(-c2ccc(C(=O)N3CCN(C(=O)C4(O)CC4)CC3)cc2)c1] |
| 29 | 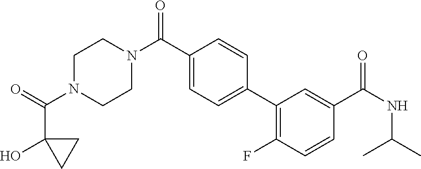 [SMILES: CC(C)NC(=O)c1ccc(F)c(-c2ccc(C(=O)N3CCN(C(=O)C4(O)CC4)CC3)cc2)c1] |
| 30 | 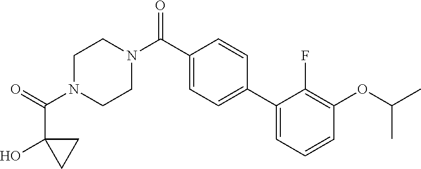 [SMILES: CC(C)Oc1cccc(-c2ccc(C(=O)N3CCN(C(=O)C4(O)CC4)CC3)cc2)c1F] |
| 31 | 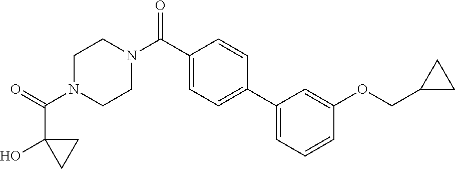 [SMILES: O=C(c1ccc(-c2cccc(OCC3CC3)c2)cc1)N1CCN(C(=O)C2(O)CC2)CC1] |
| 32 | 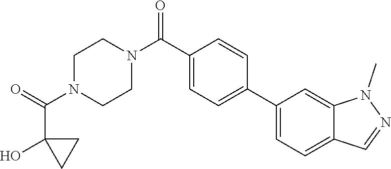 [SMILES: Cn1ncc2ccc(-c3ccc(C(=O)N4CCN(C(=O)C5(O)CC5)CC4)cc3)cc21] |
| 33 | 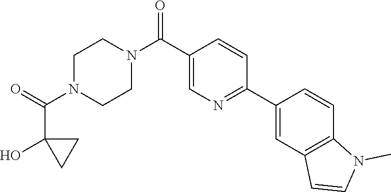 [SMILES: Cn1ccc2cc(-c3ccc(C(=O)N4CCN(C(=O)C5(O)CC5)CC4)cn3)ccc21] |
| 34 | 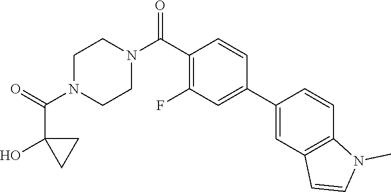 [SMILES: Cn1ccc2cc(-c3ccc(C(=O)N4CCN(C(=O)C5(O)CC5)CC4)c(F)c3)ccc21] |
| 35 | 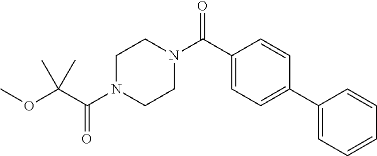 [SMILES: COC(C)(C)C(=O)N1CCN(C(=O)c2ccc(-c3ccccc3)cc2)CC1] |
| 36 | 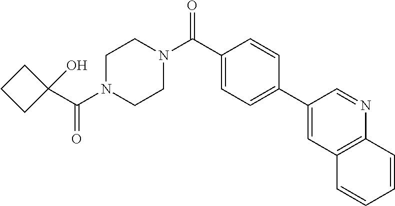 [SMILES: O=C(c1ccc(-c2cnc3ccccc3c2)cc1)N1CCN(C(=O)C2(O)CCC2)CC1] |
| 37 | 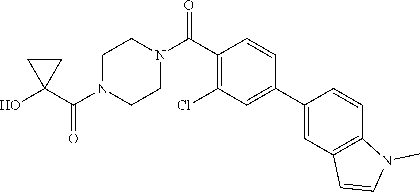 [SMILES: Cn1ccc2cc(-c3ccc(C(=O)N4CCN(C(=O)C5(O)CC5)CC4)c(Cl)c3)ccc21] |
| 38 | 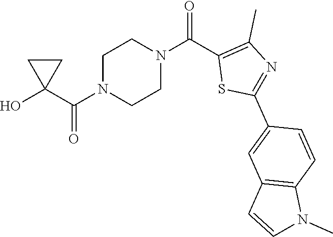 [SMILES: Cc1nc(-c2ccc3c(ccn3C)c2)sc1C(=O)N1CCN(C(=O)C2(O)CC2)CC1] |
| 39 | 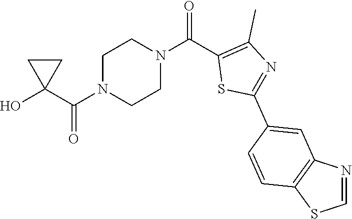 [SMILES: Cc1nc(-c2ccc3scnc3c2)sc1C(=O)N1CCN(C(=O)C2(O)CC2)CC1] |
| 40 | 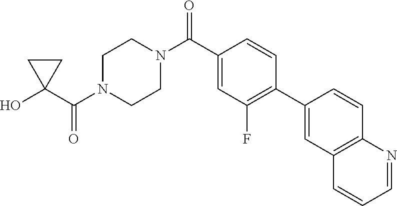 [SMILES: O=C(c1ccc(-c2ccc3ncccc3c2)c(F)c1)N1CCN(C(=O)C2(O)CC2)CC1] |
| 41 | 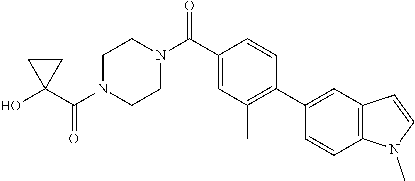 [SMILES: Cc1cc(C(=O)N2CCN(C(=O)C3(O)CC3)CC2)ccc1-c1ccc2c(ccn2C)c1] |
| 42 | 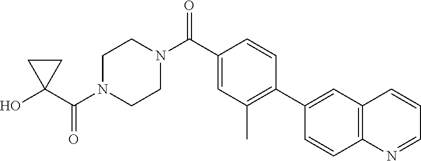 [SMILES: Cc1cc(C(=O)N2CCN(C(=O)C3(O)CC3)CC2)ccc1-c1ccc2ncccc2c1] |
| 43 | 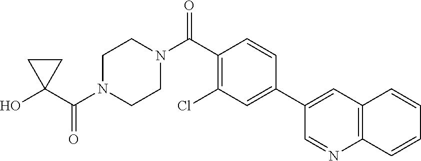 [SMILES: O=C(c1ccc(-c2cnc3ccccc3c2)cc1Cl)N1CCN(C(=O)C2(O)CC2)CC1] |
| 44 | 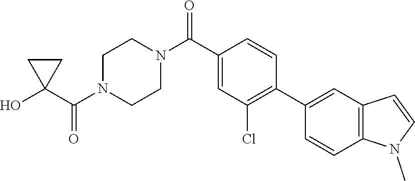 [SMILES: Cn1ccc2cc(-c3ccc(C(=O)N4CCN(C(=O)C5(O)CC5)CC4)cc3Cl)ccc21] |
| 45 | 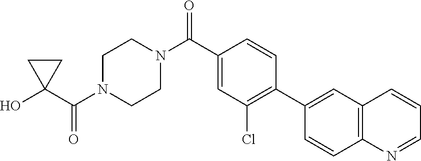 [SMILES: O=C(c1ccc(-c2ccc3ncccc3c2)c(Cl)c1)N1CCN(C(=O)C2(O)CC2)CC1] |
| 46 | 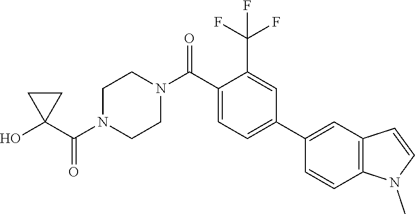 [SMILES: Cn1ccc2cc(-c3ccc(C(=O)N4CCN(C(=O)C5(O)CC5)CC4)c(C(F)(F)F)c3)ccc21] |
| 47 | 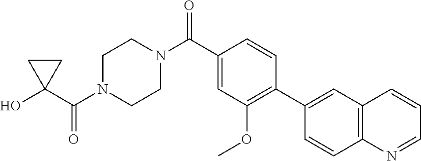 [SMILES: COc1cc(C(=O)N2CCN(C(=O)C3(O)CC3)CC2)ccc1-c1ccc2ncccc2c1] |
| 48 | 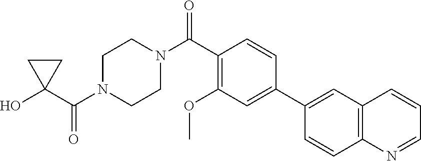 [SMILES: COc1cc(-c2ccc3ncccc3c2)ccc1C(=O)N1CCN(C(=O)C2(O)CC2)CC1] |
| 49 | 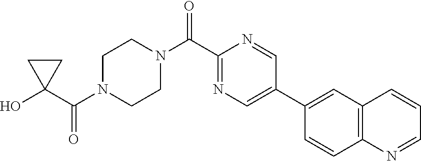 [SMILES: O=C(c1ncc(-c2ccc3ncccc3c2)cn1)N1CCN(C(=O)C2(O)CC2)CC1] |
| 50 | 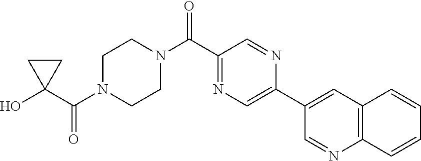 [SMILES: O=C(c1cnc(-c2cnc3ccccc3c2)cn1)N1CCN(C(=O)C2(O)CC2)CC1] |
| 51 | 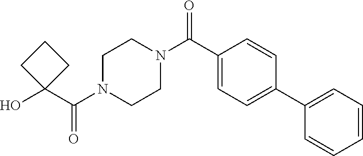 [SMILES: O=C(c1ccc(-c2ccccc2)cc1)N1CCN(C(=O)C2(O)CCC2)CC1] |
| 52 | 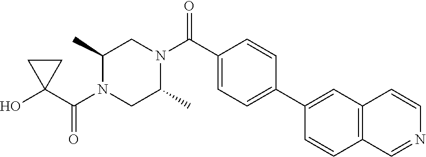 [SMILES: C[C@@H]1CN(C(=O)C2(O)CC2)[C@@H](C)CN1C(=O)c1ccc(-c2ccc3cnccc3c2)cc1] |
| 53 | 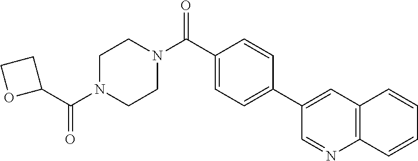 [SMILES: O=C(c1ccc(-c2cnc3ccccc3c2)cc1)N1CCN(C(=O)C2CCO2)CC1] |
| 54 | 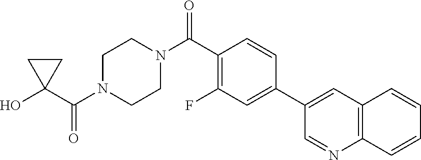 [SMILES: O=C(c1ccc(-c2cnc3ccccc3c2)cc1F)N1CCN(C(=O)C2(O)CC2)CC1] |
| 55 | 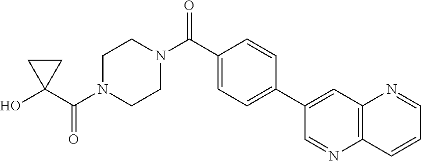 [SMILES: O=C(c1ccc(-c2cnc3cccnc3c2)cc1)N1CCN(C(=O)C2(O)CC2)CC1] |
| 56 | 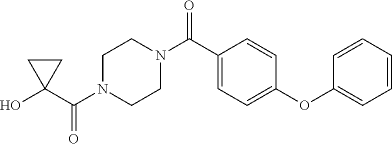 [SMILES: O=C(c1ccc(Oc2ccccc2)cc1)N1CCN(C(=O)C2(O)CC2)CC1] |
| 57 | 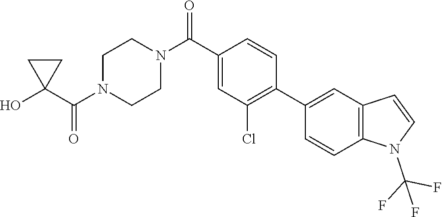 [SMILES: O=C(c1ccc(-c2ccc3c(ccn3C(F)(F)F)c2)c(Cl)c1)N1CCN(C(=O)C2(O)CC2)CC1] |
| 58 | 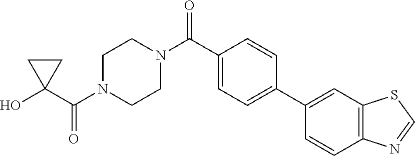 [SMILES: O=C(c1ccc(-c2ccc3ncsc3c2)cc1)N1CCN(C(=O)C2(O)CC2)CC1] |
| 59 | 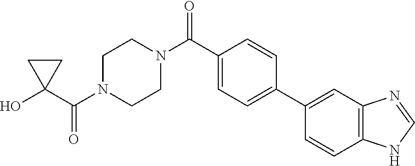 [SMILES: O=C(c1ccc(-c2ccc3[nH]cnc3c2)cc1)N1CCN(C(=O)C2(O)CC2)CC1] |
| 60 | 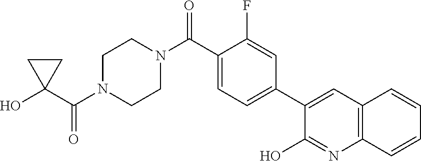 [SMILES: O=C(c1ccc(-c2cc3ccccc3nc2O)cc1F)N1CCN(C(=O)C2(O)CC2)CC1] |
| 61 | 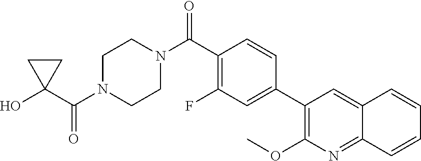 [SMILES: COc1nc2ccccc2cc1-c1ccc(C(=O)N2CCN(C(=O)C3(O)CC3)CC2)c(F)c1] |
| 62 | 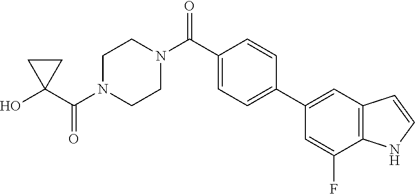 [SMILES: O=C(c1ccc(-c2cc(F)c3[nH]ccc3c2)cc1)N1CCN(C(=O)C2(O)CC2)CC1] |
| 63 | 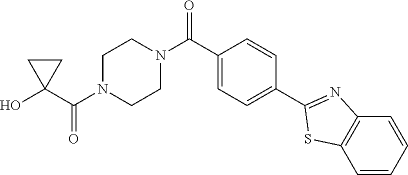 [SMILES: O=C(c1ccc(-c2nc3ccccc3s2)cc1)N1CCN(C(=O)C2(O)CC2)CC1] |
| 64 | 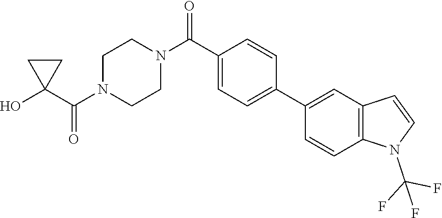 [SMILES: O=C(c1ccc(-c2ccc3c(ccn3C(F)(F)F)c2)cc1)N1CCN(C(=O)C2(O)CC2)CC1] |
| 65 | 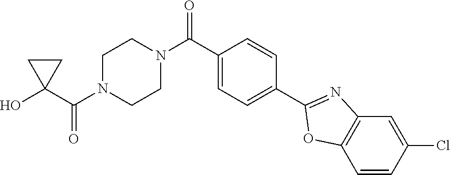 [SMILES: O=C(c1ccc(-c2nc3cc(Cl)ccc3o2)cc1)N1CCN(C(=O)C2(O)CC2)CC1] |
| 66 | 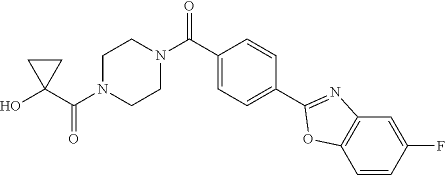 [SMILES: O=C(c1ccc(-c2nc3cc(F)ccc3o2)cc1)N1CCN(C(=O)C2(O)CC2)CC1] |
| 67 | 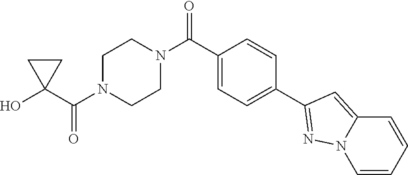 [SMILES: O=C(c1ccc(-c2cc3ccccn3n2)cc1)N1CCN(C(=O)C2(O)CC2)CC1] |
| 68 | 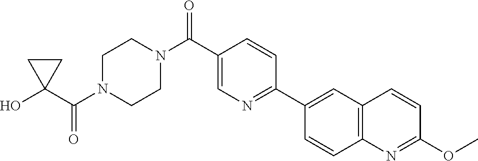 [SMILES: COc1ccc2cc(-c3ccc(C(=O)N4CCN(C(=O)C5(O)CC5)CC4)cn3)ccc2n1] |
| 69 | 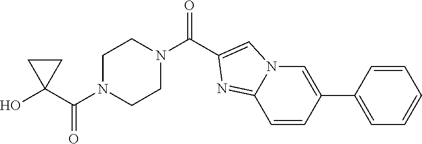 [SMILES: O=C(c1cn2cc(-c3ccccc3)ccc2n1)N1CCN(C(=O)C2(O)CC2)CC1] |
| 70 | 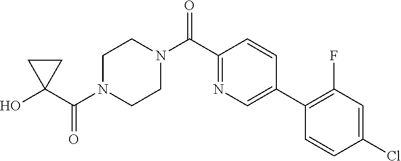 [SMILES: O=C(c1ccc(-c2ccc(Cl)cc2F)cn1)N1CCN(C(=O)C2(O)CC2)CC1] |
| 71 | 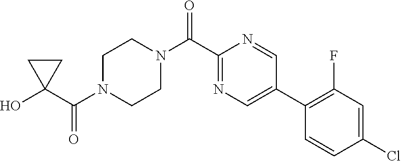 [SMILES: O=C(c1ncc(-c2ccc(Cl)cc2F)cn1)N1CCN(C(=O)C2(O)CC2)CC1] |
| 72 | 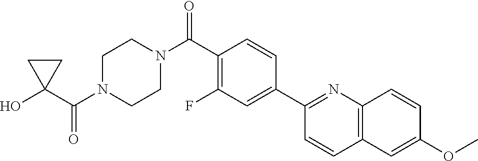 [SMILES: COc1ccc2nc(-c3ccc(C(=O)N4CCN(C(=O)C5(O)CC5)CC4)c(F)c3)ccc2c1] |
| 73 | 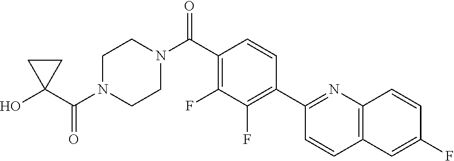 [SMILES: O=C(c1ccc(-c2ccc3cc(F)ccc3n2)c(F)c1F)N1CCN(C(=O)C2(O)CC2)CC1] |
| 74 | 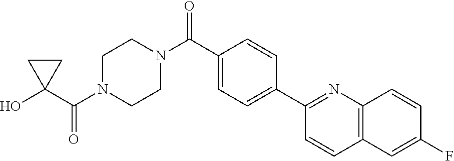 [SMILES: O=C(c1ccc(-c2ccc3cc(F)ccc3n2)cc1)N1CCN(C(=O)C2(O)CC2)CC1] |
| 75 | 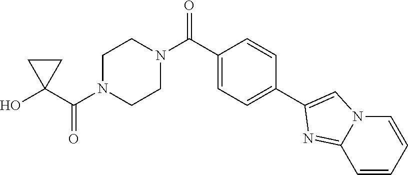 [SMILES: O=C(c1ccc(-c2cn3ccccc3n2)cc1)N1CCN(C(=O)C2(O)CC2)CC1] |
| 76 | 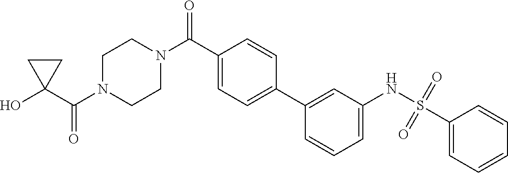 [SMILES: O=C(c1ccc(-c2cccc(NS(=O)(=O)c3ccccc3)c2)cc1)N1CCN(C(=O)C2(O)CC2)CC1] |
| 77 | 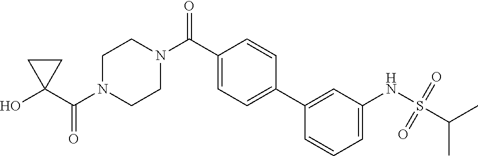 [SMILES: CC(C)S(=O)(=O)Nc1cccc(-c2ccc(C(=O)N3CCN(C(=O)C4(O)CC4)CC3)cc2)c1] |
| 78 | 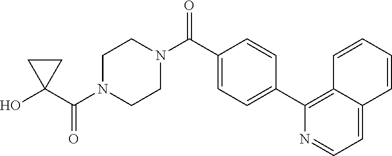 [SMILES: O=C(c1ccc(-c2nccc3ccccc23)cc1)N1CCN(C(=O)C2(O)CC2)CC1] |
| 79 | 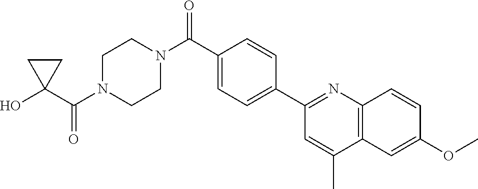 [SMILES: COc1ccc2nc(-c3ccc(C(=O)N4CCN(C(=O)C5(O)CC5)CC4)cc3)cc(C)c2c1] |
| 80 | 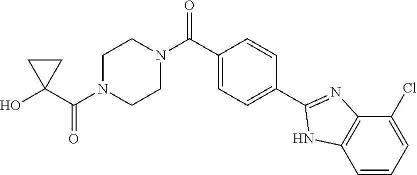 [SMILES: O=C(c1ccc(-c2nc3c(Cl)cccc3[nH]2)cc1)N1CCN(C(=O)C2(O)CC2)CC1] |
| 81 | 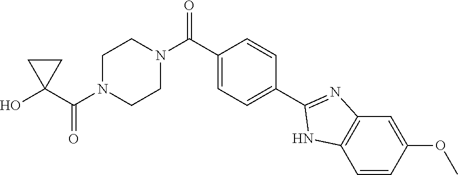 [SMILES: COc1ccc2[nH]c(-c3ccc(C(=O)N4CCN(C(=O)C5(O)CC5)CC4)cc3)nc2c1] |
| 82 | 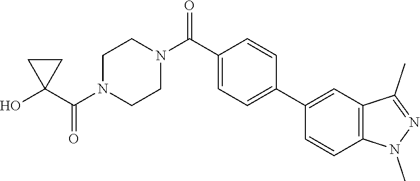 [SMILES: Cc1nn(C)c2ccc(-c3ccc(C(=O)N4CCN(C(=O)C5(O)CC5)CC4)cc3)cc12] |
| 83 | 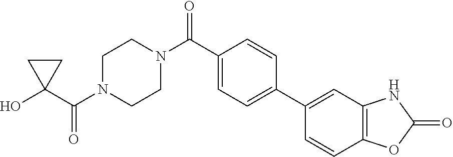 [SMILES: O=C(c1ccc(-c2ccc3oc(=O)[nH]c3c2)cc1)N1CCN(C(=O)C2(O)CC2)CC1] |
| 84 | 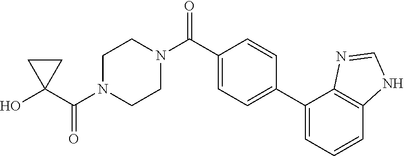 [SMILES: O=C(c1ccc(-c2cccc3[nH]cnc23)cc1)N1CCN(C(=O)C2(O)CC2)CC1] |
| 85 | 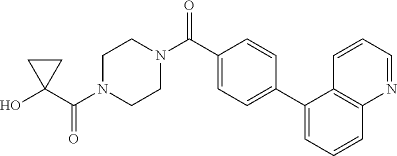 [SMILES: O=C(c1ccc(-c2cccc3ncccc23)cc1)N1CCN(C(=O)C2(O)CC2)CC1] |
| 86 | 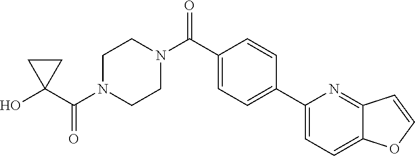 [SMILES: O=C(c1ccc(-c2ccc3occc3n2)cc1)N1CCN(C(=O)C2(O)CC2)CC1] |
| 87 | 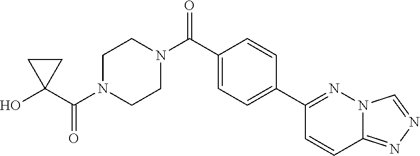 [SMILES: O=C(c1ccc(-c2ccc3nncn3n2)cc1)N1CCN(C(=O)C2(O)CC2)CC1] |
| 88 | 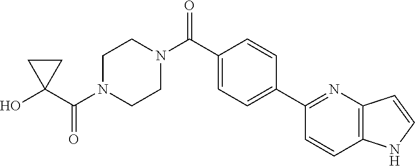 [SMILES: O=C(c1ccc(-c2ccc3[nH]ccc3n2)cc1)N1CCN(C(=O)C2(O)CC2)CC1] |
| 89 | 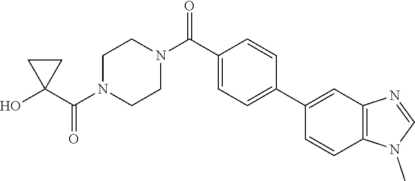 [SMILES: Cn1cnc2cc(-c3ccc(C(=O)N4CCN(C(=O)C5(O)CC5)CC4)cc3)ccc21] |
| 90 | 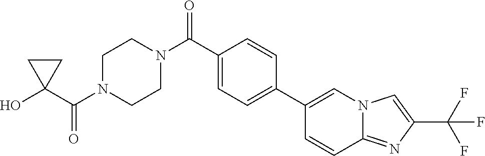 [SMILES: O=C(c1ccc(-c2ccc3nc(C(F)(F)F)cn3c2)cc1)N1CCN(C(=O)C2(O)CC2)CC1] |
| 91 | 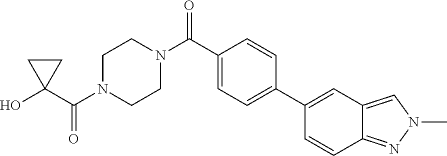 [SMILES: Cn1cc2cc(-c3ccc(C(=O)N4CCN(C(=O)C5(O)CC5)CC4)cc3)ccc2n1] |
| 92 | 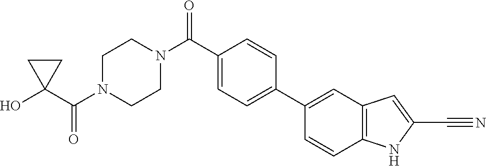 [SMILES: N#Cc1cc2cc(-c3ccc(C(=O)N4CCN(C(=O)C5(O)CC5)CC4)cc3)ccc2[nH]1] |
| 93 | 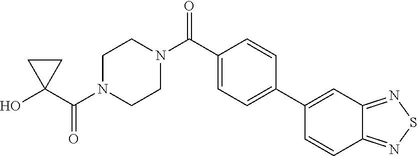 [SMILES: O=C(c1ccc(-c2ccc3nsnc3c2)cc1)N1CCN(C(=O)C2(O)CC2)CC1] |
| 94 | 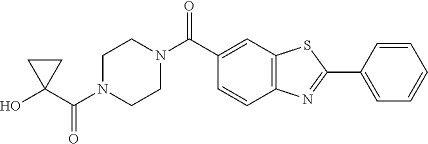 [SMILES: O=C(c1ccc2nc(-c3ccccc3)sc2c1)N1CCN(C(=O)C2(O)CC2)CC1] |
| 95 | 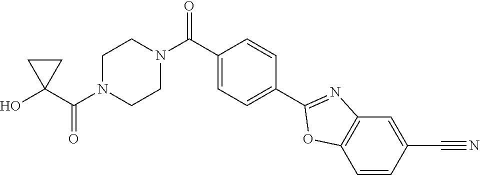 [SMILES: N#Cc1ccc2oc(-c3ccc(C(=O)N4CCN(C(=O)C5(O)CC5)CC4)cc3)nc2c1] |
| 96 | 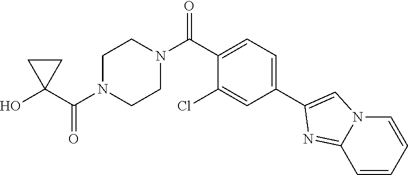 [SMILES: O=C(c1ccc(-c2cn3ccccc3n2)cc1Cl)N1CCN(C(=O)C2(O)CC2)CC1] |
| 97 | 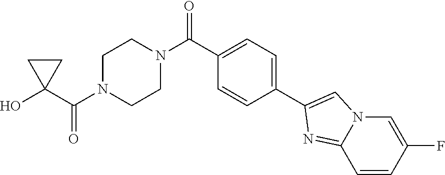 [SMILES: O=C(c1ccc(-c2cn3cc(F)ccc3n2)cc1)N1CCN(C(=O)C2(O)CC2)CC1] |
| 98 | 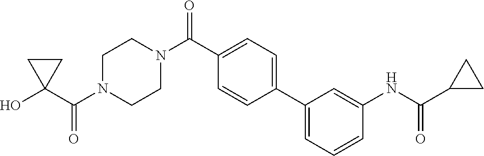 [SMILES: O=C(Nc1cccc(-c2ccc(C(=O)N3CCN(C(=O)C4(O)CC4)CC3)cc2)c1)C1CC1] |
| 99 | 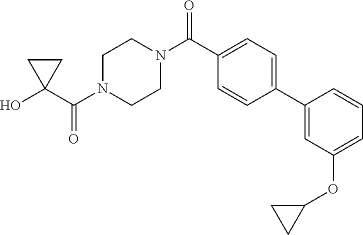 [SMILES: O=C(c1ccc(-c2cccc(OC3CC3)c2)cc1)N1CCN(C(=O)C2(O)CC2)CC1] |
| 100 | 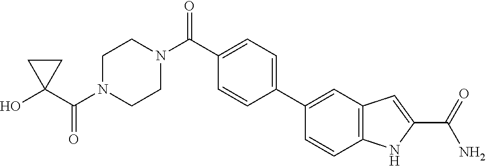 [SMILES: NC(=O)c1cc2cc(-c3ccc(C(=O)N4CCN(C(=O)C5(O)CC5)CC4)cc3)ccc2[nH]1] |
| 101 | 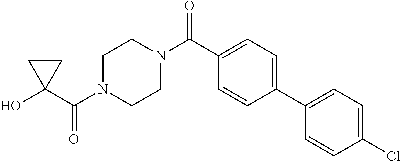 [SMILES: O=C(c1ccc(-c2ccc(Cl)cc2)cc1)N1CCN(C(=O)C2(O)CC2)CC1] |
| 102 | 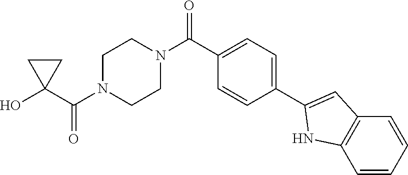 [SMILES: O=C(c1ccc(-c2cc3ccccc3[nH]2)cc1)N1CCN(C(=O)C2(O)CC2)CC1] |
| 103 | 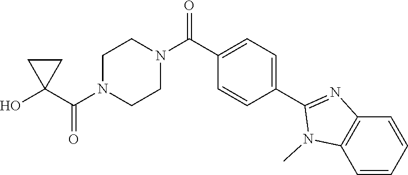 [SMILES: Cn1c(-c2ccc(C(=O)N3CCN(C(=O)C4(O)CC4)CC3)cc2)nc2ccccc21] |
| 104 | 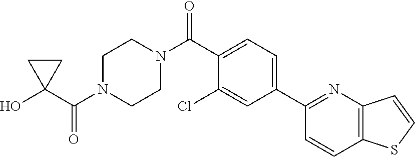 [SMILES: O=C(c1ccc(-c2ccc3sccc3n2)cc1Cl)N1CCN(C(=O)C2(O)CC2)CC1] |
| 105 | 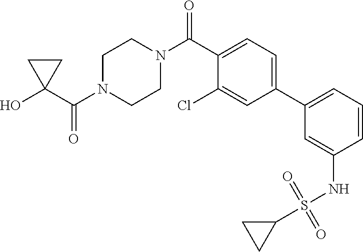 [SMILES: O=C(c1ccc(-c2cccc(NS(=O)(=O)C3CC3)c2)cc1Cl)N1CCN(C(=O)C2(O)CC2)CC1] |
| 106 | 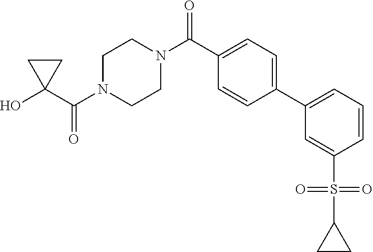 [SMILES: O=C(c1ccc(-c2cccc(S(=O)(=O)C3CC3)c2)cc1)N1CCN(C(=O)C2(O)CC2)CC1] |
| 107 | 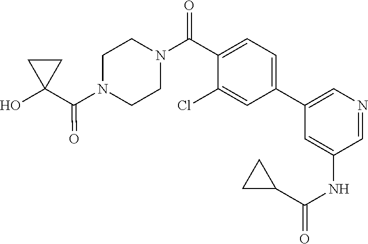 [SMILES: O=C(Nc1cncc(-c2ccc(C(=O)N3CCN(C(=O)C4(O)CC4)CC3)c(Cl)c2)c1)C1CC1] |
| 108 | 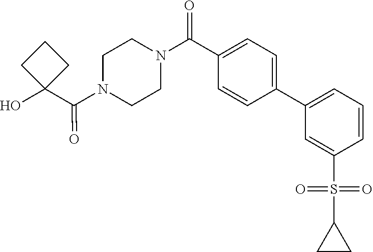 [SMILES: O=C(c1ccc(-c2cccc(S(=O)(=O)C3CC3)c2)cc1)N1CCN(C(=O)C2(O)CCC2)CC1] |
| 109 | 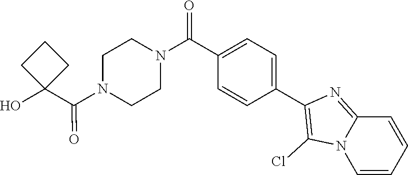 [SMILES: O=C(c1ccc(-c2nc3ccccn3c2Cl)cc1)N1CCN(C(=O)C2(O)CCC2)CC1] |
| 110 | 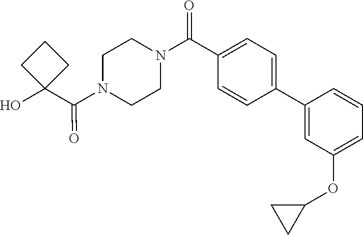 [SMILES: O=C(c1ccc(-c2cccc(OC3CC3)c2)cc1)N1CCN(C(=O)C2(O)CCC2)CC1] |
| 111 | 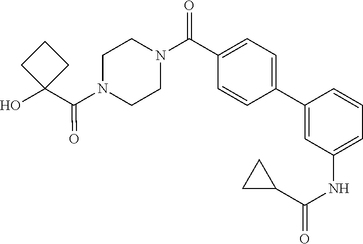 [SMILES: O=C(Nc1cccc(-c2ccc(C(=O)N3CCN(C(=O)C4(O)CCC4)CC3)cc2)c1)C1CC1] |
| 112 | 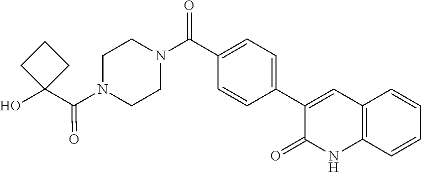 [SMILES: O=C(c1ccc(-c2cc3ccccc3[nH]c2=O)cc1)N1CCN(C(=O)C2(O)CCC2)CC1] |
| 113 | 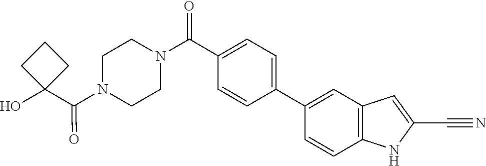 [SMILES: N#Cc1cc2cc(-c3ccc(C(=O)N4CCN(C(=O)C5(O)CCC5)CC4)cc3)ccc2[nH]1] |
| 114 | 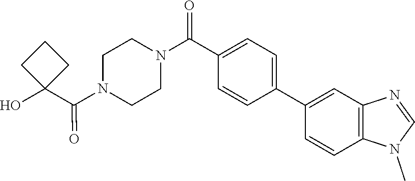 [SMILES: Cn1cnc2cc(-c3ccc(C(=O)N4CCN(C(=O)C5(O)CCC5)CC4)cc3)ccc21] |
| 115 | 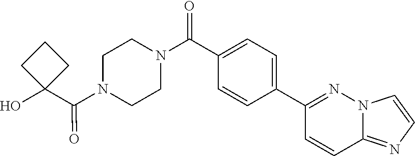 [SMILES: O=C(c1ccc(-c2ccc3nccn3n2)cc1)N1CCN(C(=O)C2(O)CCC2)CC1] |
| 116 | 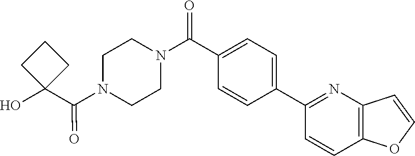 [SMILES: O=C(c1ccc(-c2ccc3occc3n2)cc1)N1CCN(C(=O)C2(O)CCC2)CC1] |
| 117 | 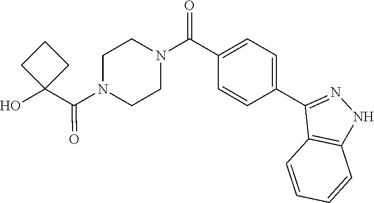 [SMILES: O=C(c1ccc(-c2n[nH]c3ccccc23)cc1)N1CCN(C(=O)C2(O)CCC2)CC1] |
| 118 | 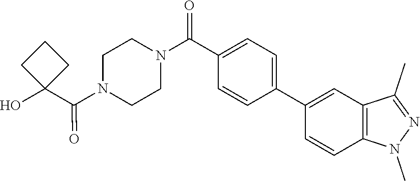 [SMILES: Cc1nn(C)c2ccc(-c3ccc(C(=O)N4CCN(C(=O)C5(O)CCC5)CC4)cc3)cc12] |
| 119 | 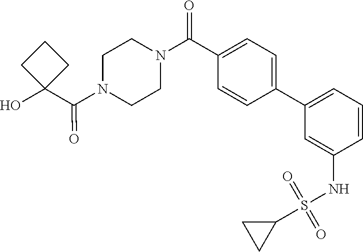 [SMILES: O=C(c1ccc(-c2cccc(NS(=O)(=O)C3CC3)c2)cc1)N1CCN(C(=O)C2(O)CCC2)CC1] |
| 120 | 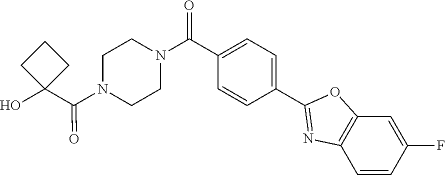 [SMILES: O=C(c1ccc(-c2nc3ccc(F)cc3o2)cc1)N1CCN(C(=O)C2(O)CCC2)CC1] |
| 121 | 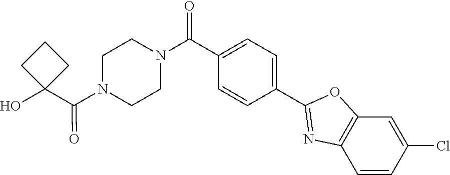 [SMILES: O=C(c1ccc(-c2nc3ccc(Cl)cc3o2)cc1)N1CCN(C(=O)C2(O)CCC2)CC1] |
| 122 | 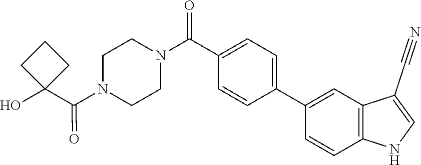 [SMILES: N#Cc1c[nH]c2ccc(-c3ccc(C(=O)N4CCN(C(=O)C5(O)CCC5)CC4)cc3)cc12] |
| 123 | 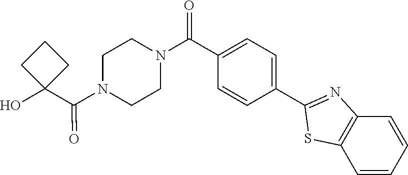 [SMILES: O=C(c1ccc(-c2nc3ccccc3s2)cc1)N1CCN(C(=O)C2(O)CCC2)CC1] |
| 124 | 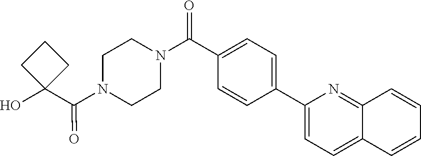 [SMILES: O=C(c1ccc(-c2ccc3ccccc3n2)cc1)N1CCN(C(=O)C2(O)CCC2)CC1] |
| 125 | 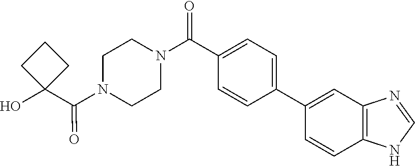 [SMILES: O=C(c1ccc(-c2ccc3[nH]cnc3c2)cc1)N1CCN(C(=O)C2(O)CCC2)CC1] |
| 126 | 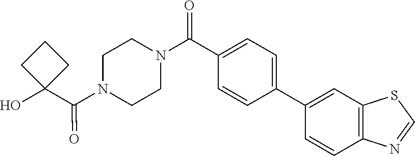 [SMILES: O=C(c1ccc(-c2ccc3ncsc3c2)cc1)N1CCN(C(=O)C2(O)CCC2)CC1] |
| 127 | 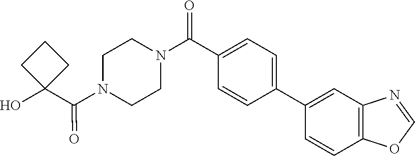 [SMILES: O=C(c1ccc(-c2ccc3ocnc3c2)cc1)N1CCN(C(=O)C2(O)CCC2)CC1] |
| 128 | 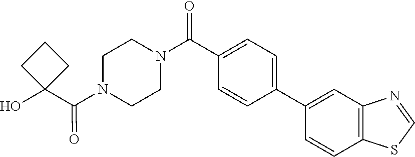 [SMILES: O=C(c1ccc(-c2ccc3scnc3c2)cc1)N1CCN(C(=O)C2(O)CCC2)CC1] |
| 129 | 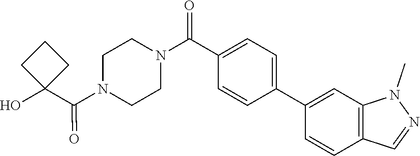 [SMILES: Cn1ncc2ccc(-c3ccc(C(=O)N4CCN(C(=O)C5(O)CCC5)CC4)cc3)cc21] |
| 130 | 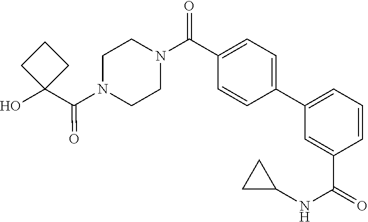 [SMILES: O=C(NC1CC1)c1cccc(-c2ccc(C(=O)N3CCN(C(=O)C4(O)CCC4)CC3)cc2)c1] |
| 131 | 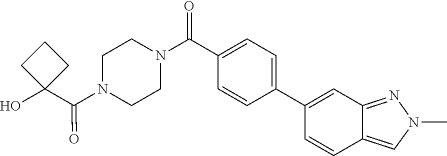 [SMILES: Cn1cc2ccc(-c3ccc(C(=O)N4CCN(C(=O)C5(O)CCC5)CC4)cc3)cc2n1] |
| 132 | 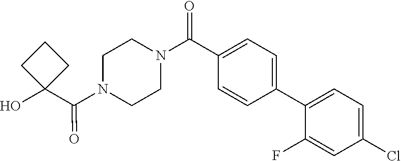 [SMILES: O=C(c1ccc(-c2ccc(Cl)cc2F)cc1)N1CCN(C(=O)C2(O)CCC2)CC1] |
| 133 | 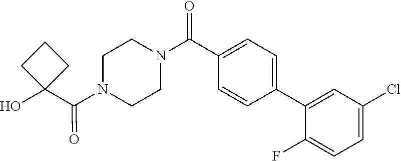 [SMILES: O=C(c1ccc(-c2cc(Cl)ccc2F)cc1)N1CCN(C(=O)C2(O)CCC2)CC1] |
| 134 | 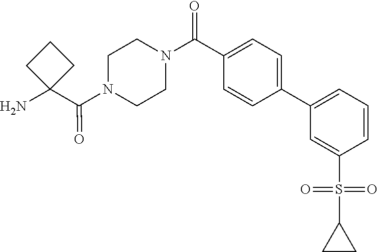 [SMILES: NC1(C(=O)N2CCN(C(=O)c3ccc(-c4cccc(S(=O)(=O)C5CC5)c4)cc3)CC2)CCC1] |
| 135 | 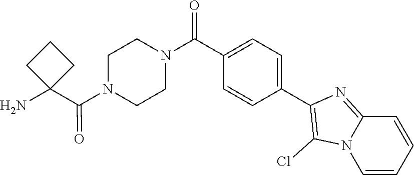 [SMILES: NC1(C(=O)N2CCN(C(=O)c3ccc(-c4nc5ccccn5c4Cl)cc3)CC2)CCC1] |
| 136 | 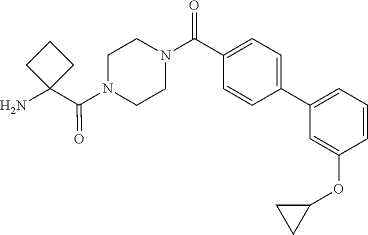 [SMILES: NC1(C(=O)N2CCN(C(=O)c3ccc(-c4cccc(OC5CC5)c4)cc3)CC2)CCC1] |
| 137 | 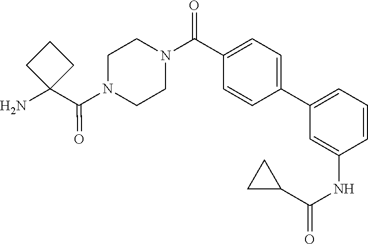 [SMILES: NC1(C(=O)N2CCN(C(=O)c3ccc(-c4cccc(NC(=O)C5CC5)c4)cc3)CC2)CCC1] |
| 138 | 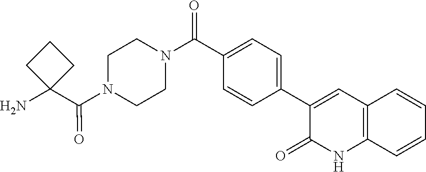 [SMILES: NC1(C(=O)N2CCN(C(=O)c3ccc(-c4cc5ccccc5[nH]c4=O)cc3)CC2)CCC1] |
| 139 | 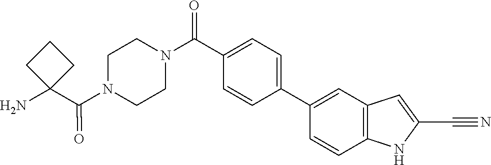 [SMILES: N#Cc1cc2cc(-c3ccc(C(=O)N4CCN(C(=O)C5(N)CCC5)CC4)cc3)ccc2[nH]1] |
| 140 | 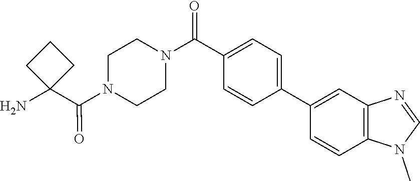 [SMILES: Cn1cnc2cc(-c3ccc(C(=O)N4CCN(C(=O)C5(N)CCC5)CC4)cc3)ccc21] |
| 141 | 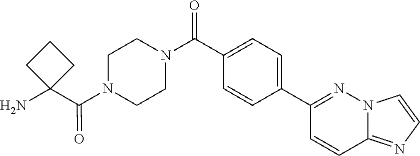 [SMILES: NC1(C(=O)N2CCN(C(=O)c3ccc(-c4ccc5nccn5n4)cc3)CC2)CCC1] |
| 142 | 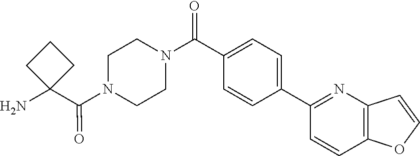 [SMILES: NC1(C(=O)N2CCN(C(=O)c3ccc(-c4ccc5occc5n4)cc3)CC2)CCC1] |
| 143 | 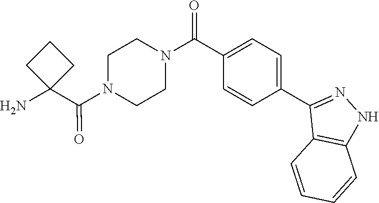 [SMILES: NC1(C(=O)N2CCN(C(=O)c3ccc(-c4n[nH]c5ccccc45)cc3)CC2)CCC1] |
| 144 | 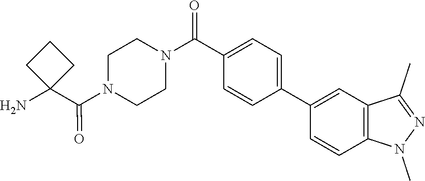 [SMILES: Cc1nn(C)c2ccc(-c3ccc(C(=O)N4CCN(C(=O)C5(N)CCC5)CC4)cc3)cc12] |
| 145 | 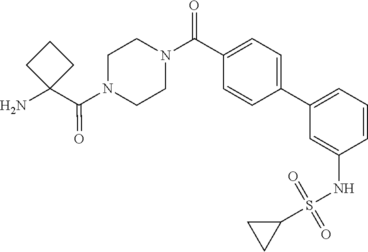 [SMILES: NC1(C(=O)N2CCN(C(=O)c3ccc(-c4cccc(NS(=O)(=O)C5CC5)c4)cc3)CC2)CCC1] |
| 146 | 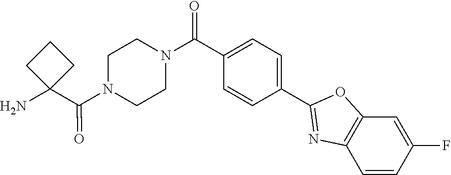 [SMILES: NC1(C(=O)N2CCN(C(=O)c3ccc(-c4nc5ccc(F)cc5o4)cc3)CC2)CCC1] |
| 147 | 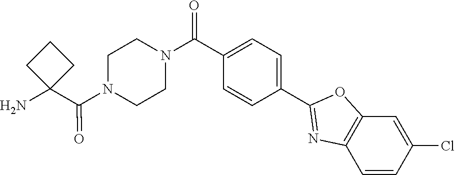 [SMILES: NC1(C(=O)N2CCN(C(=O)c3ccc(-c4nc5ccc(Cl)cc5o4)cc3)CC2)CCC1] |
| 148 | 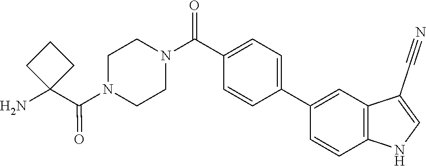 [SMILES: N#Cc1c[nH]c2ccc(-c3ccc(C(=O)N4CCN(C(=O)C5(N)CCC5)CC4)cc3)cc12] |
| 149 | 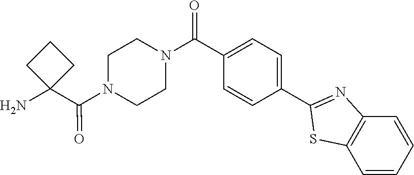 [SMILES: NC1(C(=O)N2CCN(C(=O)c3ccc(-c4nc5ccccc5s4)cc3)CC2)CCC1] |
| 150 | 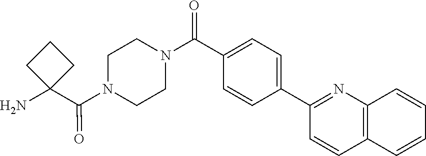 [SMILES: NC1(C(=O)N2CCN(C(=O)c3ccc(-c4ccc5ccccc5n4)cc3)CC2)CCC1] |
| 151 | 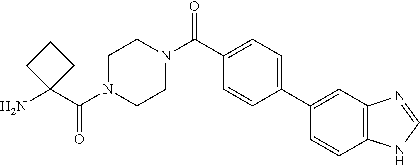 [SMILES: NC1(C(=O)N2CCN(C(=O)c3ccc(-c4ccc5[nH]cnc5c4)cc3)CC2)CCC1] |
| 152 | 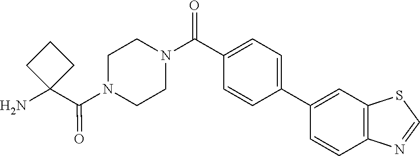 [SMILES: NC1(C(=O)N2CCN(C(=O)c3ccc(-c4ccc5ncsc5c4)cc3)CC2)CCC1] |
| 153 | 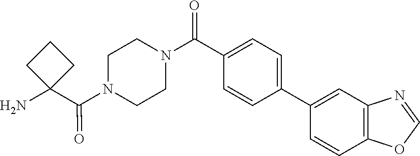 [SMILES: NC1(C(=O)N2CCN(C(=O)c3ccc(-c4ccc5ocnc5c4)cc3)CC2)CCC1] |
| 154 | 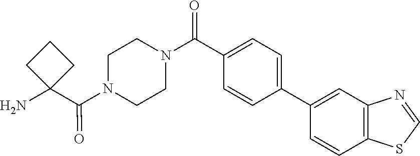 [SMILES: NC1(C(=O)N2CCN(C(=O)c3ccc(-c4ccc5scnc5c4)cc3)CC2)CCC1] |
| 155 | 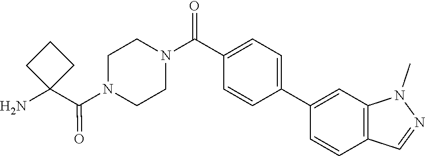 [SMILES: Cn1ncc2ccc(-c3ccc(C(=O)N4CCN(C(=O)C5(N)CCC5)CC4)cc3)cc21] |
| 156 | 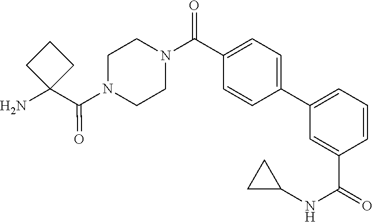 [SMILES: NC1(C(=O)N2CCN(C(=O)c3ccc(-c4cccc(C(=O)NC5CC5)c4)cc3)CC2)CCC1] |
| 157 | 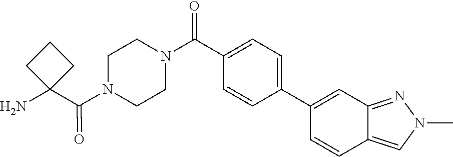 [SMILES: Cn1cc2ccc(-c3ccc(C(=O)N4CCN(C(=O)C5(N)CCC5)CC4)cc3)cc2n1] |
| 158 | 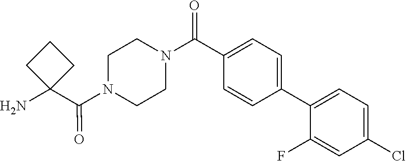 [SMILES: NC1(C(=O)N2CCN(C(=O)c3ccc(-c4ccc(Cl)cc4F)cc3)CC2)CCC1] |
| 159 | 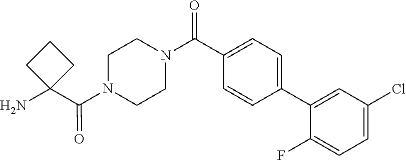 [SMILES: NC1(C(=O)N2CCN(C(=O)c3ccc(-c4cc(Cl)ccc4F)cc3)CC2)CCC1] |
| 160 | 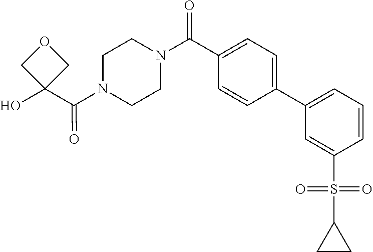 [SMILES: O=C(c1ccc(-c2cccc(S(=O)(=O)C3CC3)c2)cc1)N1CCN(C(=O)C2(O)COC2)CC1] |
| 161 | 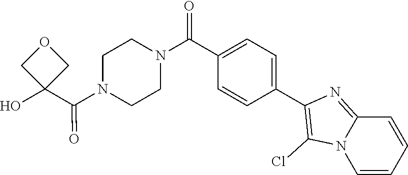 [SMILES: O=C(c1ccc(-c2nc3ccccn3c2Cl)cc1)N1CCN(C(=O)C2(O)COC2)CC1] |
| 162 | 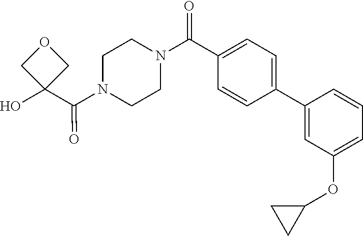 [SMILES: O=C(c1ccc(-c2cccc(OC3CC3)c2)cc1)N1CCN(C(=O)C2(O)COC2)CC1] |
| 163 | 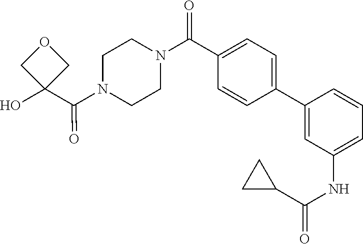 [SMILES: O=C(Nc1cccc(-c2ccc(C(=O)N3CCN(C(=O)C4(O)COC4)CC3)cc2)c1)C1CC1] |
| 164 | 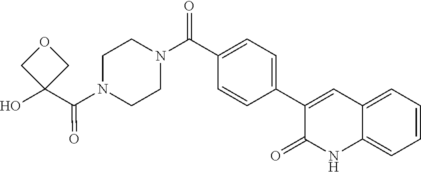 [SMILES: O=C(c1ccc(-c2cc3ccccc3[nH]c2=O)cc1)N1CCN(C(=O)C2(O)COC2)CC1] |
| 165 | 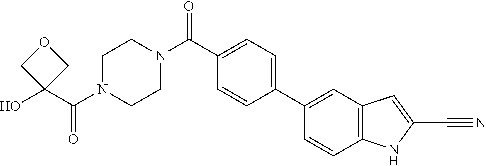 [SMILES: N#Cc1cc2cc(-c3ccc(C(=O)N4CCN(C(=O)C5(O)COC5)CC4)cc3)ccc2[nH]1] |
| 166 | 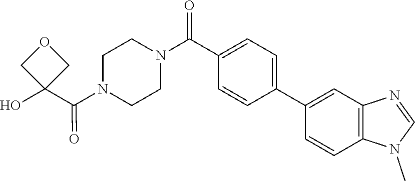 [SMILES: Cn1cnc2cc(-c3ccc(C(=O)N4CCN(C(=O)C5(O)COC5)CC4)cc3)ccc21] |
| 167 | 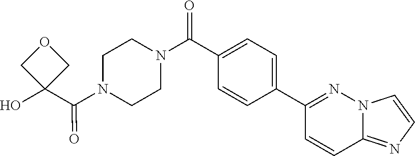 [SMILES: O=C(c1ccc(-c2ccc3nccn3n2)cc1)N1CCN(C(=O)C2(O)COC2)CC1] |
| 168 | 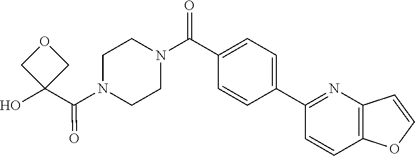 [SMILES: O=C(c1ccc(-c2ccc3occc3n2)cc1)N1CCN(C(=O)C2(O)COC2)CC1] |
| 169 | 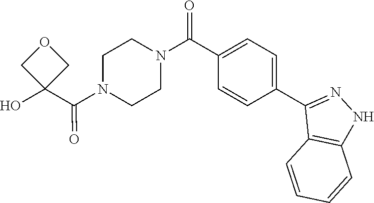 [SMILES: O=C(c1ccc(-c2n[nH]c3ccccc23)cc1)N1CCN(C(=O)C2(O)COC2)CC1] |
| 170 | 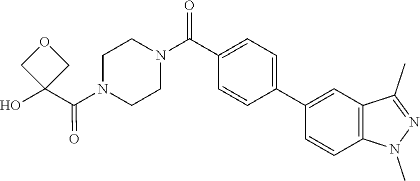 [SMILES: Cc1nn(C)c2ccc(-c3ccc(C(=O)N4CCN(C(=O)C5(O)COC5)CC4)cc3)cc12] |
| 171 | 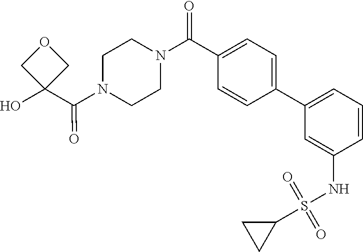 [SMILES: O=C(c1ccc(-c2cccc(NS(=O)(=O)C3CC3)c2)cc1)N1CCN(C(=O)C2(O)COC2)CC1] |
| 172 | 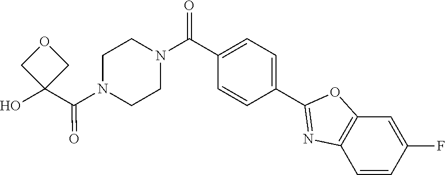 [SMILES: O=C(c1ccc(-c2nc3ccc(F)cc3o2)cc1)N1CCN(C(=O)C2(O)COC2)CC1] |
| 173 | 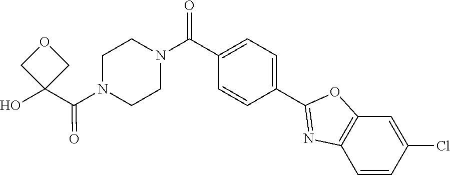 [SMILES: O=C(c1ccc(-c2nc3ccc(Cl)cc3o2)cc1)N1CCN(C(=O)C2(O)COC2)CC1] |
| 174 | 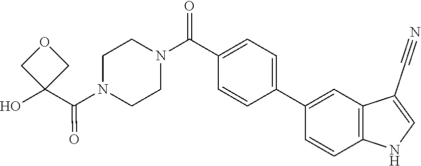 [SMILES: N#Cc1c[nH]c2ccc(-c3ccc(C(=O)N4CCN(C(=O)C5(O)COC5)CC4)cc3)cc12] |
| 175 | 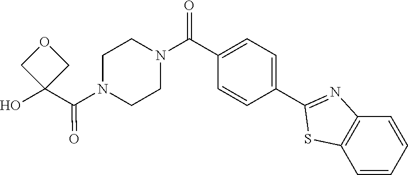 [SMILES: O=C(c1ccc(-c2nc3ccccc3s2)cc1)N1CCN(C(=O)C2(O)COC2)CC1] |
| 176 | 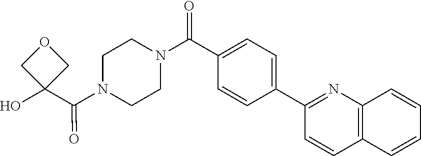 [SMILES: O=C(c1ccc(-c2ccc3ccccc3n2)cc1)N1CCN(C(=O)C2(O)COC2)CC1] |
| 177 | 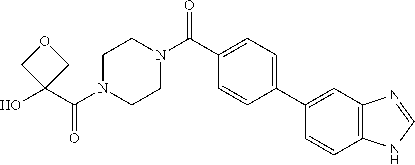 [SMILES: O=C(c1ccc(-c2ccc3[nH]cnc3c2)cc1)N1CCN(C(=O)C2(O)COC2)CC1] |
| 178 | 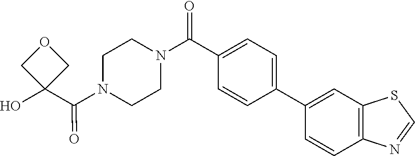 [SMILES: O=C(c1ccc(-c2ccc3ncsc3c2)cc1)N1CCN(C(=O)C2(O)COC2)CC1] |
| 179 | 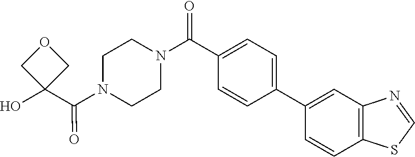 [SMILES: O=C(c1ccc(-c2ccc3scnc3c2)cc1)N1CCN(C(=O)C2(O)COC2)CC1] |
| 180 | 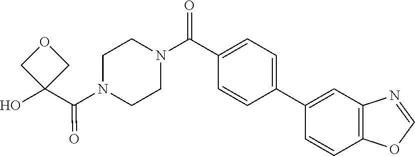 [SMILES: O=C(c1ccc(-c2ccc3ocnc3c2)cc1)N1CCN(C(=O)C2(O)COC2)CC1] |
| 181 | 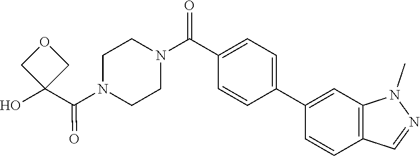 [SMILES: Cn1ncc2ccc(-c3ccc(C(=O)N4CCN(C(=O)C5(O)COC5)CC4)cc3)cc21] |
| 182 | 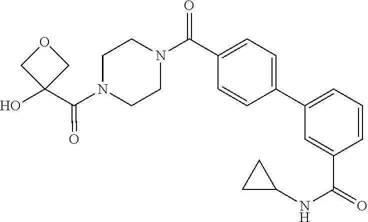 [SMILES: O=C(NC1CC1)c1cccc(-c2ccc(C(=O)N3CCN(C(=O)C4(O)COC4)CC3)cc2)c1] |
| 183 | 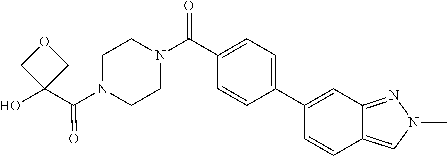 [SMILES: Cn1cc2ccc(-c3ccc(C(=O)N4CCN(C(=O)C5(O)COC5)CC4)cc3)cc2n1] |
| 184 | 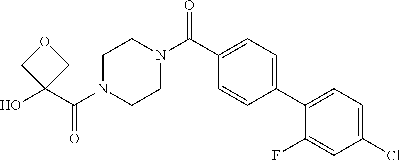 [SMILES: O=C(c1ccc(-c2ccc(Cl)cc2F)cc1)N1CCN(C(=O)C2(O)COC2)CC1] |
| 185 | 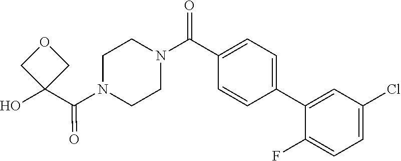 [SMILES: O=C(c1ccc(-c2cc(Cl)ccc2F)cc1)N1CCN(C(=O)C2(O)COC2)CC1] |
| 186 | 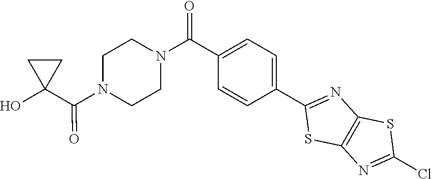 [SMILES: O=C(c1ccc(-c2nc3sc(Cl)nc3s2)cc1)N1CCN(C(=O)C2(O)CC2)CC1] |
| 187 | 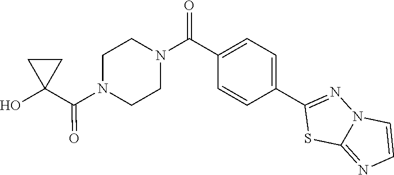 [SMILES: O=C(c1ccc(-c2nn3ccnc3s2)cc1)N1CCN(C(=O)C2(O)CC2)CC1] |
| 188 | 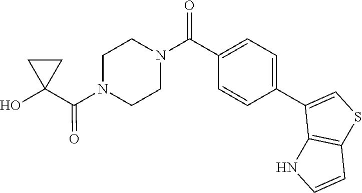 [SMILES: O=C(c1ccc(-c2csc3cc[nH]c23)cc1)N1CCN(C(=O)C2(O)CC2)CC1] |
| 189 | 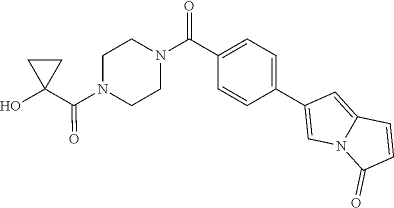 [SMILES: O=C(c1ccc(-c2cc3n(c2)C(=O)C=C3)cc1)N1CCN(C(=O)C2(O)CC2)CC1] |
| 190 | 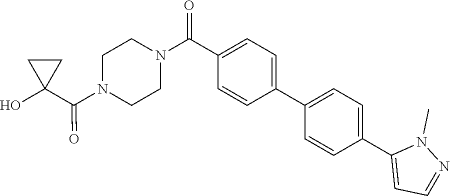 [SMILES: Cn1nccc1-c1ccc(-c2ccc(C(=O)N3CCN(C(=O)C4(O)CC4)CC3)cc2)cc1] |
| 191 | 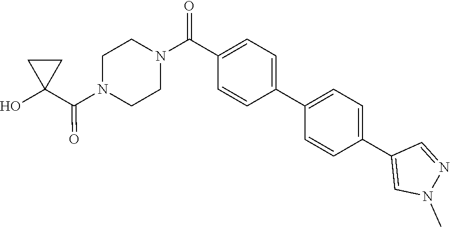 [SMILES: Cn1cc(-c2ccc(-c3ccc(C(=O)N4CCN(C(=O)C5(O)CC5)CC4)cc3)cc2)cn1] |
| 192 | 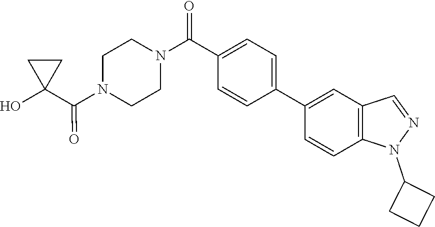 [SMILES: O=C(c1ccc(-c2ccc3c(cnn3C3CCC3)c2)cc1)N1CCN(C(=O)C2(O)CC2)CC1] |
| 193 | 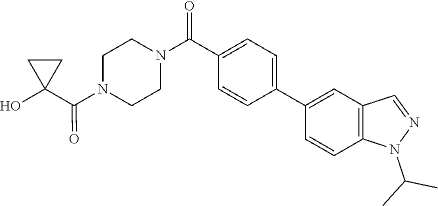 [SMILES: CC(C)n1ncc2cc(-c3ccc(C(=O)N4CCN(C(=O)C5(O)CC5)CC4)cc3)ccc21] |
| 194 | 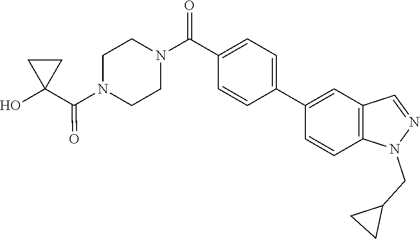 [SMILES: O=C(c1ccc(-c2ccc3c(cnn3CC3CC3)c2)cc1)N1CCN(C(=O)C2(O)CC2)CC1] |
| 195 | 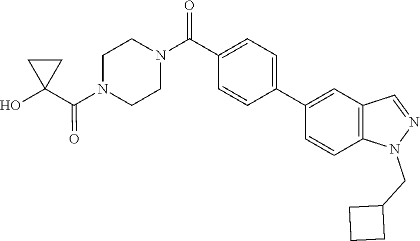 [SMILES: O=C(c1ccc(-c2ccc3c(cnn3CC3CCC3)c2)cc1)N1CCN(C(=O)C2(O)CC2)CC1] |
| 196 | 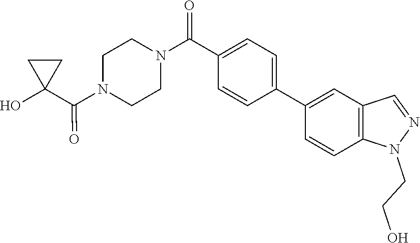 [SMILES: O=C(c1ccc(-c2ccc3c(cnn3CCO)c2)cc1)N1CCN(C(=O)C2(O)CC2)CC1] |
| 197 | 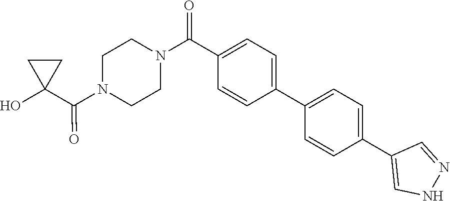 [SMILES: O=C(c1ccc(-c2ccc(-c3cn[nH]c3)cc2)cc1)N1CCN(C(=O)C2(O)CC2)CC1] |
| 198 | 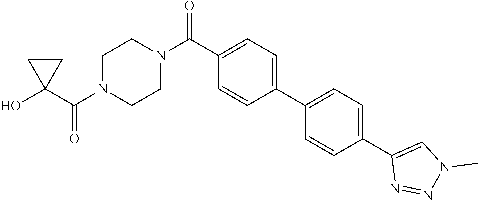 [SMILES: Cn1cc(-c2ccc(-c3ccc(C(=O)N4CCN(C(=O)C5(O)CC5)CC4)cc3)cc2)nn1] |
| 199 | 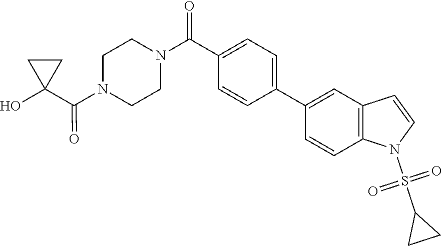 [SMILES: O=C(c1ccc(-c2ccc3c(ccn3S(=O)(=O)C3CC3)c2)cc1)N1CCN(C(=O)C2(O)CC2)CC1] |
| 200 | 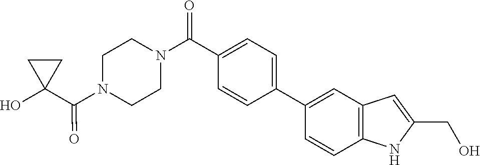 [SMILES: O=C(c1ccc(-c2ccc3[nH]c(CO)cc3c2)cc1)N1CCN(C(=O)C2(O)CC2)CC1] |
| 201 | 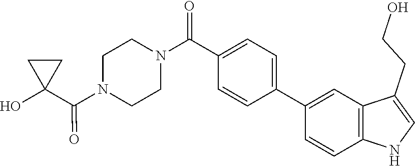 [SMILES: O=C(c1ccc(-c2ccc3[nH]cc(CCO)c3c2)cc1)N1CCN(C(=O)C2(O)CC2)CC1] |
| 202 | 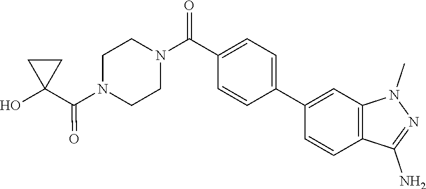 [SMILES: Cn1nc(N)c2ccc(-c3ccc(C(=O)N4CCN(C(=O)C5(O)CC5)CC4)cc3)cc21] |
| 203 | 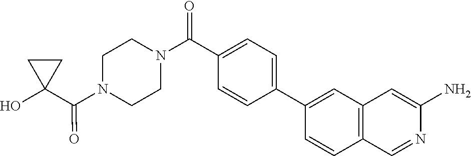 [SMILES: Nc1cc2cc(-c3ccc(C(=O)N4CCN(C(=O)C5(O)CC5)CC4)cc3)ccc2cn1] |
| 204 | 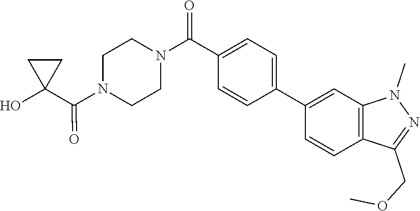 [SMILES: COCc1nn(C)c2cc(-c3ccc(C(=O)N4CCN(C(=O)C5(O)CC5)CC4)cc3)ccc12] |
| 205 | 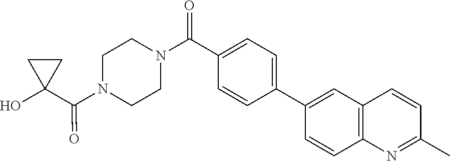 [SMILES: Cc1ccc2cc(-c3ccc(C(=O)N4CCN(C(=O)C5(O)CC5)CC4)cc3)ccc2n1] |
| 206 | 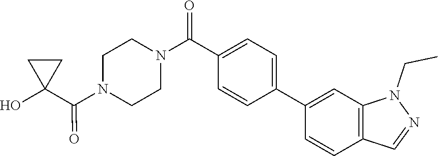 [SMILES: CCn1ncc2ccc(-c3ccc(C(=O)N4CCN(C(=O)C5(O)CC5)CC4)cc3)cc21] |
| 207 | 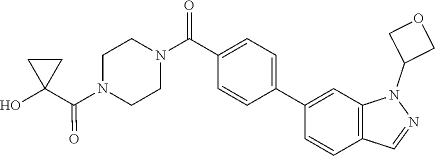 [SMILES: O=C(c1ccc(-c2ccc3cnn(C4COC4)c3c2)cc1)N1CCN(C(=O)C2(O)CC2)CC1] |
| 208 | 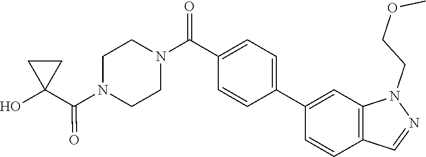 [SMILES: COCCn1ncc2ccc(-c3ccc(C(=O)N4CCN(C(=O)C5(O)CC5)CC4)cc3)cc21] |
| 209 | 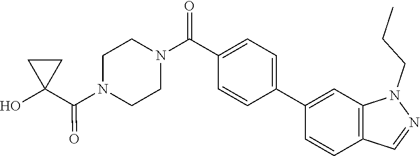 [SMILES: CCCn1ncc2ccc(-c3ccc(C(=O)N4CCN(C(=O)C5(O)CC5)CC4)cc3)cc21] |
| 210 | 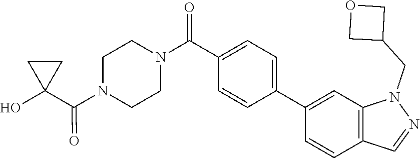 [SMILES: O=C(c1ccc(-c2ccc3cnn(CC4COC4)c3c2)cc1)N1CCN(C(=O)C2(O)CC2)CC1] |
| 211 | 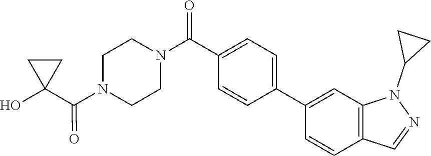 [SMILES: O=C(c1ccc(-c2ccc3cnn(C4CC4)c3c2)cc1)N1CCN(C(=O)C2(O)CC2)CC1] |
| 212 | 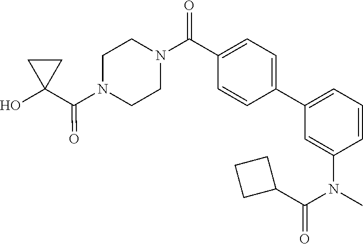 [SMILES: CN(C(=O)C1CCC1)c1cccc(-c2ccc(C(=O)N3CCN(C(=O)C4(O)CC4)CC3)cc2)c1] |
| 213 | 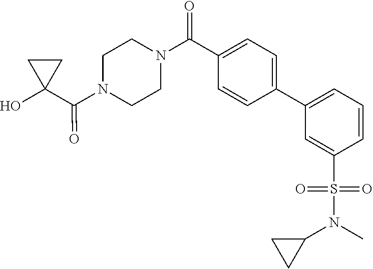 [SMILES: CN(C1CC1)S(=O)(=O)c1cccc(-c2ccc(C(=O)N3CCN(C(=O)C4(O)CC4)CC3)cc2)c1] |
| 214 | 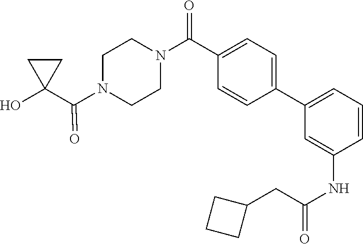 [SMILES: O=C(CC1CCC1)Nc1cccc(-c2ccc(C(=O)N3CCN(C(=O)C4(O)CC4)CC3)cc2)c1] |
| 215 | 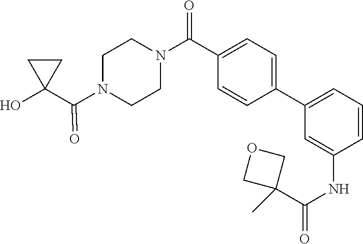 [SMILES: CC1(C(=O)Nc2cccc(-c3ccc(C(=O)N4CCN(C(=O)C5(O)CC5)CC4)cc3)c2)COC1] |
| 216 | 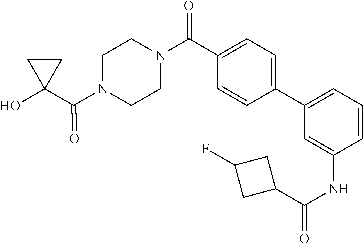 [SMILES: O=C(Nc1cccc(-c2ccc(C(=O)N3CCN(C(=O)C4(O)CC4)CC3)cc2)c1)C1CC(F)C1] |
| 217 | 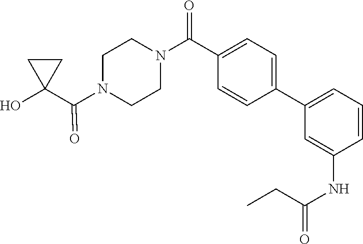 [SMILES: CCC(=O)Nc1cccc(-c2ccc(C(=O)N3CCN(C(=O)C4(O)CC4)CC3)cc2)c1] |
| 218 | 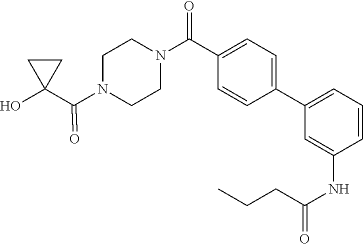 [SMILES: CCCC(=O)Nc1cccc(-c2ccc(C(=O)N3CCN(C(=O)C4(O)CC4)CC3)cc2)c1] |
| 219 | 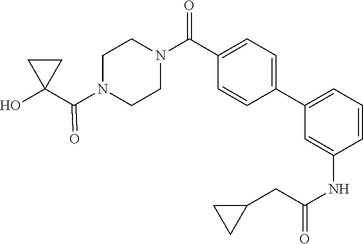 [SMILES: O=C(CC1CC1)Nc1cccc(-c2ccc(C(=O)N3CCN(C(=O)C4(O)CC4)CC3)cc2)c1] |
| 220 | 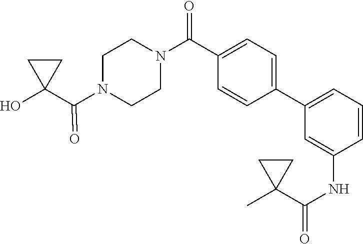 [SMILES: CC1(C(=O)Nc2cccc(-c3ccc(C(=O)N4CCN(C(=O)C5(O)CC5)CC4)cc3)c2)CC1] |
| 221 | 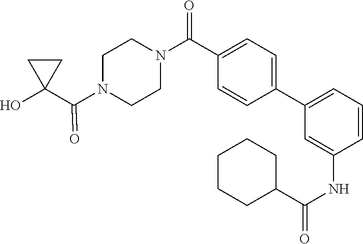 [SMILES: O=C(Nc1cccc(-c2ccc(C(=O)N3CCN(C(=O)C4(O)CC4)CC3)cc2)c1)C1CCCCC1] |
| 222 | 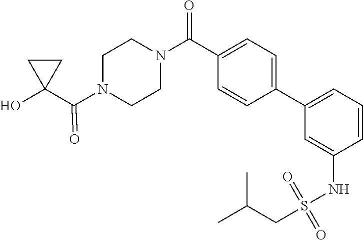 [SMILES: CC(C)CS(=O)(=O)Nc1cccc(-c2ccc(C(=O)N3CCN(C(=O)C4(O)CC4)CC3)cc2)c1] |
| 223 | 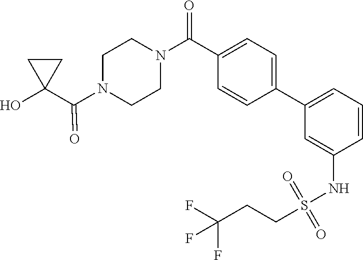 [SMILES: O=C(c1ccc(-c2cccc(NS(=O)(=O)CCC(F)(F)F)c2)cc1)N1CCN(C(=O)C2(O)CC2)CC1] |
| 224 | 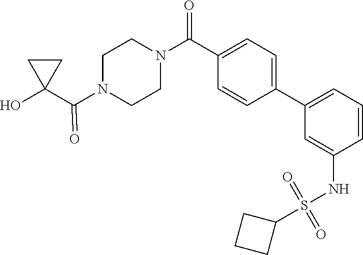 [SMILES: O=C(c1ccc(-c2cccc(NS(=O)(=O)C3CCC3)c2)cc1)N1CCN(C(=O)C2(O)CC2)CC1] |
| 225 | 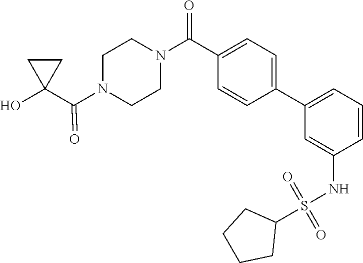 [SMILES: O=C(c1ccc(-c2cccc(NS(=O)(=O)C3CCCC3)c2)cc1)N1CCN(C(=O)C2(O)CC2)CC1] |
| 226 | 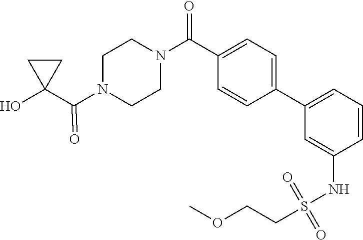 [SMILES: COCCS(=O)(=O)Nc1cccc(-c2ccc(C(=O)N3CCN(C(=O)C4(O)CC4)CC3)cc2)c1] |
| 227 | 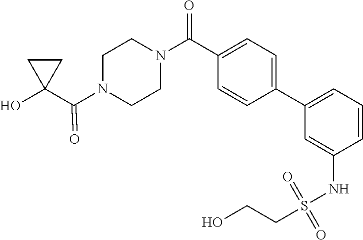 [SMILES: O=C(c1ccc(-c2cccc(NS(=O)(=O)CCO)c2)cc1)N1CCN(C(=O)C2(O)CC2)CC1] |
| 228 | 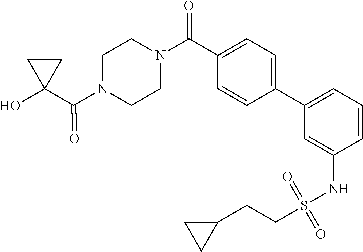 [SMILES: O=C(c1ccc(-c2cccc(NS(=O)(=O)CCC3CC3)c2)cc1)N1CCN(C(=O)C2(O)CC2)CC1] |
| 229 | 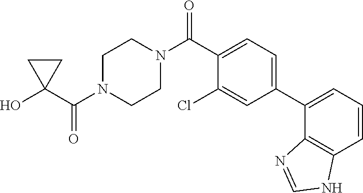 [SMILES: O=C(c1ccc(-c2cccc3[nH]cnc23)cc1Cl)N1CCN(C(=O)C2(O)CC2)CC1] |
| 230 | 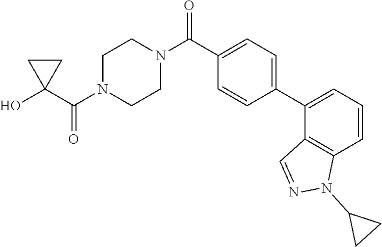 [SMILES: O=C(c1ccc(-c2cccc3c2cnn3C2CC2)cc1)N1CCN(C(=O)C2(O)CC2)CC1] |
| 231 | 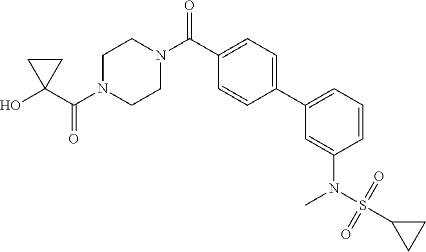 [SMILES: CN(c1cccc(-c2ccc(C(=O)N3CCN(C(=O)C4(O)CC4)CC3)cc2)c1)S(=O)(=O)C1CC1] |
| 232 | 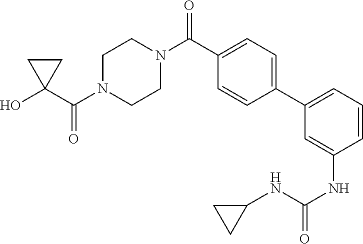 [SMILES: O=C(Nc1cccc(-c2ccc(C(=O)N3CCN(C(=O)C4(O)CC4)CC3)cc2)c1)NC1CC1] |
| 233 | 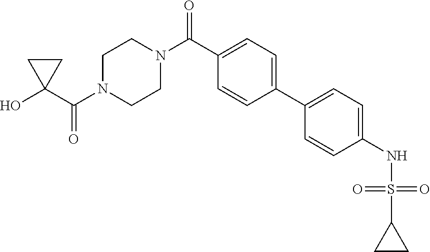 [SMILES: O=C(c1ccc(-c2ccc(NS(=O)(=O)C3CC3)cc2)cc1)N1CCN(C(=O)C2(O)CC2)CC1] |
| 234 | 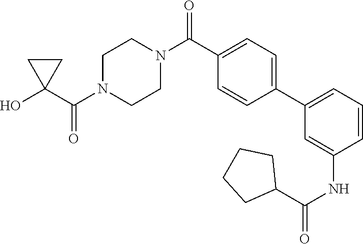 [SMILES: O=C(Nc1cccc(-c2ccc(C(=O)N3CCN(C(=O)C4(O)CC4)CC3)cc2)c1)C1CCCC1] |
| 235 | 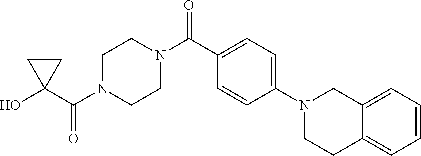 [SMILES: O=C(c1ccc(N2CCc3ccccc3C2)cc1)N1CCN(C(=O)C2(O)CC2)CC1] |
| 236 | 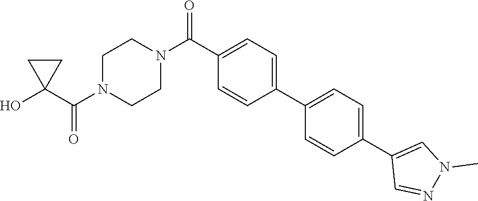 [SMILES: Cn1cc(-c2ccc(-c3ccc(C(=O)N4CCN(C(=O)C5(O)CC5)CC4)cc3)cc2)cn1] |
| 237 | 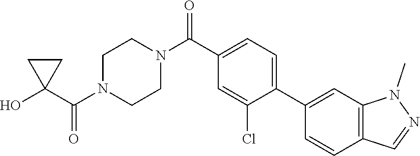 [SMILES: Cn1ncc2ccc(-c3ccc(C(=O)N4CCN(C(=O)C5(O)CC5)CC4)cc3Cl)cc21] |
| 238 | 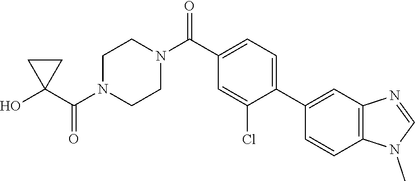 [SMILES: Cn1cnc2cc(-c3ccc(C(=O)N4CCN(C(=O)C5(O)CC5)CC4)cc3Cl)ccc21] |
| 239 | 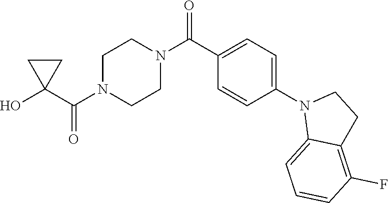 [SMILES: O=C(c1ccc(N2CCc3c(F)cccc32)cc1)N1CCN(C(=O)C2(O)CC2)CC1] |
| 240 | 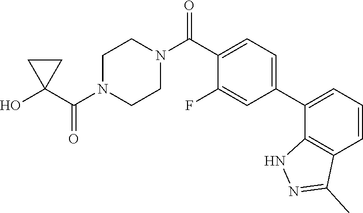 [SMILES: Cc1n[nH]c2c(-c3ccc(C(=O)N4CCN(C(=O)C5(O)CC5)CC4)c(F)c3)cccc12] |
| 241 | 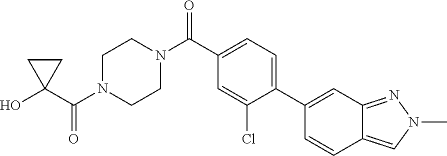 [SMILES: Cn1cc2ccc(-c3ccc(C(=O)N4CCN(C(=O)C5(O)CC5)CC4)cc3Cl)cc2n1] |
| 242 | 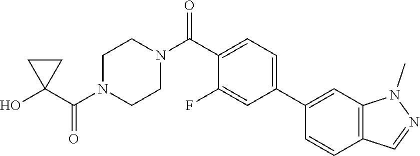 [SMILES: Cn1ncc2ccc(-c3ccc(C(=O)N4CCN(C(=O)C5(O)CC5)CC4)c(F)c3)cc21] |
| 243 | 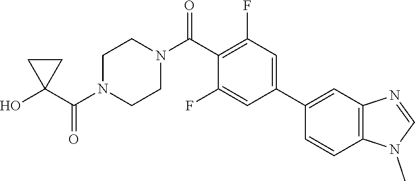 [SMILES: Cn1cnc2cc(-c3cc(F)c(C(=O)N4CCN(C(=O)C5(O)CC5)CC4)c(F)c3)ccc21] |
| 244 | 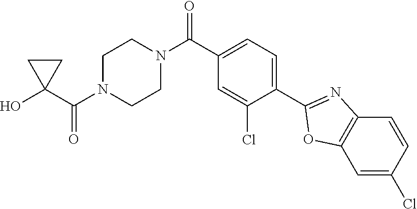 [SMILES: O=C(c1ccc(-c2nc3ccc(Cl)cc3o2)c(Cl)c1)N1CCN(C(=O)C2(O)CC2)CC1] |
| 245 | 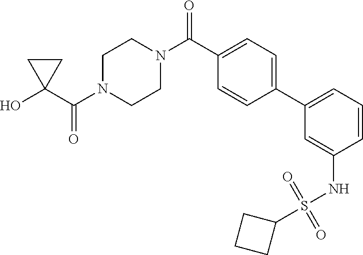 [SMILES: O=C(c1ccc(-c2cccc(NS(=O)(=O)C3CCC3)c2)cc1)N1CCN(C(=O)C2(O)CC2)CC1] |
| 246 | 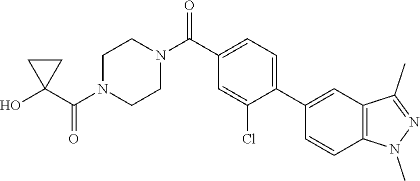 [SMILES: Cc1nn(C)c2ccc(-c3ccc(C(=O)N4CCN(C(=O)C5(O)CC5)CC4)cc3Cl)cc12] |
| 247 | 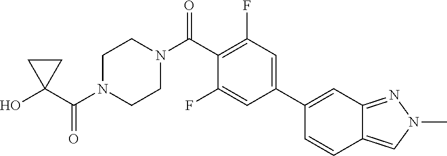 [SMILES: Cn1cc2ccc(-c3cc(F)c(C(=O)N4CCN(C(=O)C5(O)CC5)CC4)c(F)c3)cc2n1] |
| 248 | 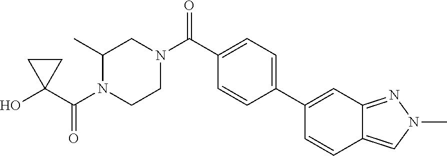 [SMILES: CC1CN(C(=O)c2ccc(-c3ccc4cn(C)nc4c3)cc2)CCN1C(=O)C1(O)CC1] |
| 249 | 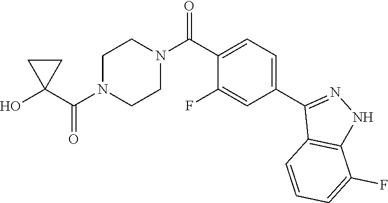 [SMILES: O=C(c1ccc(-c2n[nH]c3c(F)cccc23)cc1F)N1CCN(C(=O)C2(O)CC2)CC1] |
| 250 | 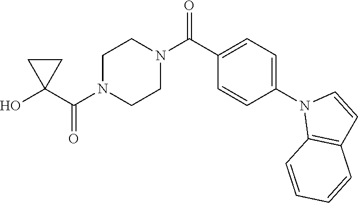 [SMILES: O=C(c1ccc(-n2ccc3ccccc32)cc1)N1CCN(C(=O)C2(O)CC2)CC1] |
| 251 | 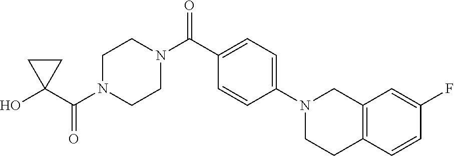 [SMILES: O=C(c1ccc(N2CCc3ccc(F)cc3C2)cc1)N1CCN(C(=O)C2(O)CC2)CC1] |
| 252 | 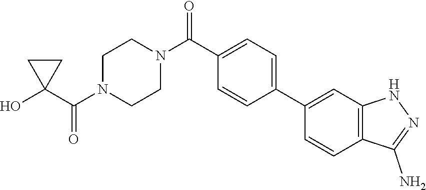 [SMILES: Nc1n[nH]c2cc(-c3ccc(C(=O)N4CCN(C(=O)C5(O)CC5)CC4)cc3)ccc12] |
| 253 | 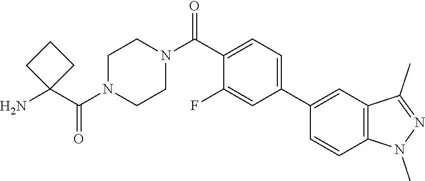 [SMILES: Cc1nn(C)c2ccc(-c3ccc(C(=O)N4CCN(C(=O)C5(N)CCC5)CC4)c(F)c3)cc12] |
| 254 | 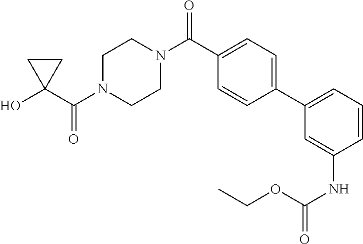 [SMILES: CCOC(=O)Nc1cccc(-c2ccc(C(=O)N3CCN(C(=O)C4(O)CC4)CC3)cc2)c1] |
| 255 | 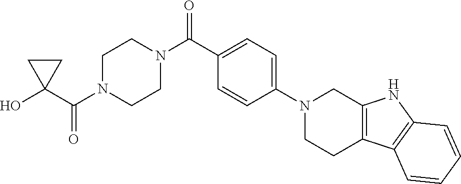 [SMILES: O=C(c1ccc(N2CCc3c([nH]c4ccccc34)C2)cc1)N1CCN(C(=O)C2(O)CC2)CC1] |
| 256 | 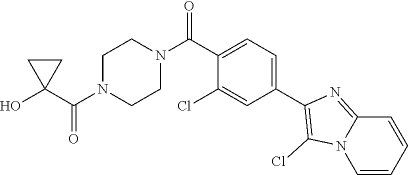 [SMILES: O=C(c1ccc(-c2nc3ccccn3c2Cl)cc1Cl)N1CCN(C(=O)C2(O)CC2)CC1] |
| 257 | 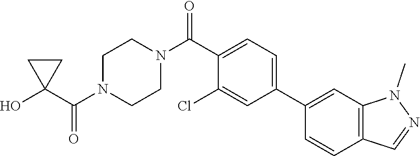 [SMILES: Cn1ncc2ccc(-c3ccc(C(=O)N4CCN(C(=O)C5(O)CC5)CC4)c(Cl)c3)cc21] |
| 258 | 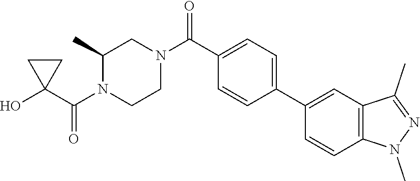 [SMILES: Cc1nn(C)c2ccc(-c3ccc(C(=O)N4CCN(C(=O)C5(O)CC5)[C@@H](C)C4)cc3)cc12] |
| 259 | 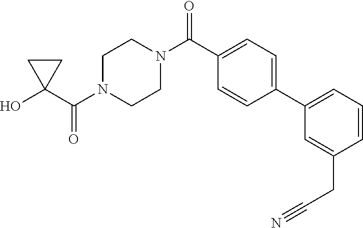 [SMILES: N#CCc1cccc(-c2ccc(C(=O)N3CCN(C(=O)C4(O)CC4)CC3)cc2)c1] |
| 260 | 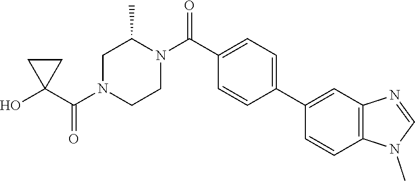 [SMILES: C[C@H]1CN(C(=O)C2(O)CC2)CCN1C(=O)c1ccc(-c2ccc3c(c2)ncn3C)cc1] |
| 261 | 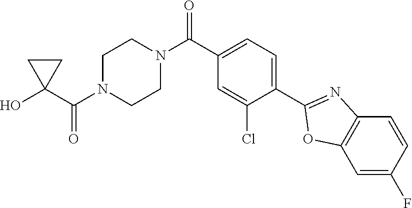 [SMILES: O=C(c1ccc(-c2nc3ccc(F)cc3o2)c(Cl)c1)N1CCN(C(=O)C2(O)CC2)CC1] |
| 262 | 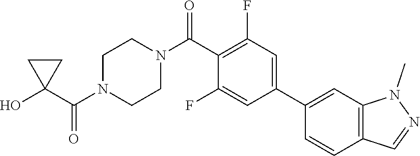 [SMILES: Cn1ncc2ccc(-c3cc(F)c(C(=O)N4CCN(C(=O)C5(O)CC5)CC4)c(F)c3)cc21] |
| 263 | 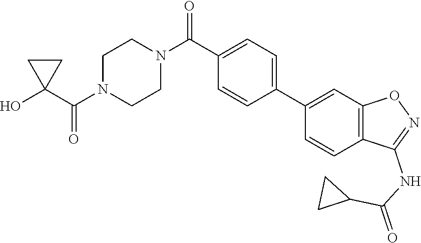 [SMILES: O=C(Nc1noc2cc(-c3ccc(C(=O)N4CCN(C(=O)C5(O)CC5)CC4)cc3)ccc12)C1CC1] |
| 264 | 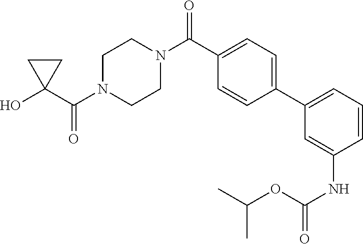 [SMILES: CC(C)OC(=O)Nc1cccc(-c2ccc(C(=O)N3CCN(C(=O)C4(O)CC4)CC3)cc2)c1] |
| 265 | 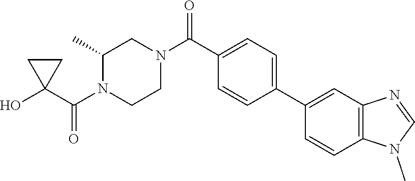 [SMILES: C[C@@H]1CN(C(=O)c2ccc(-c3ccc4c(c3)ncn4C)cc2)CCN1C(=O)C1(O)CC1] |
| 266 | 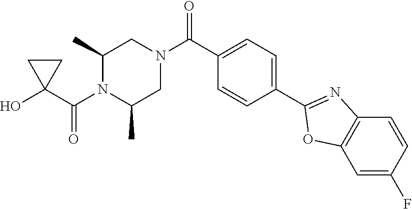 [SMILES: C[C@@H]1CN(C(=O)c2ccc(-c3nc4ccc(F)cc4o3)cc2)C[C@H](C)N1C(=O)C1(O)CC1] |
| 267 | 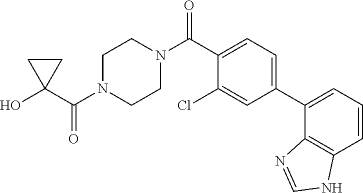 [SMILES: O=C(c1ccc(-c2cccc3[nH]cnc23)cc1Cl)N1CCN(C(=O)C2(O)CC2)CC1] |
| 268 | 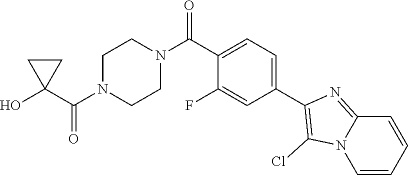 [SMILES: O=C(c1ccc(-c2nc3ccccn3c2Cl)cc1F)N1CCN(C(=O)C2(O)CC2)CC1] |
| 269 | 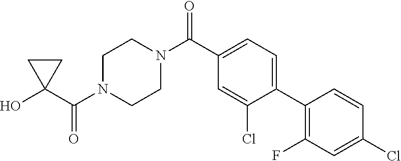 [SMILES: O=C(c1ccc(-c2ccc(Cl)cc2F)c(Cl)c1)N1CCN(C(=O)C2(O)CC2)CC1] |
| 270 | 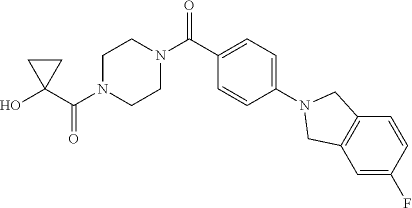 [SMILES: O=C(c1ccc(N2Cc3ccc(F)cc3C2)cc1)N1CCN(C(=O)C2(O)CC2)CC1] |
| 271 | 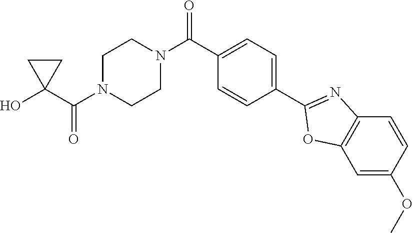 [SMILES: COc1ccc2nc(-c3ccc(C(=O)N4CCN(C(=O)C5(O)CC5)CC4)cc3)oc2c1] |
| 272 | 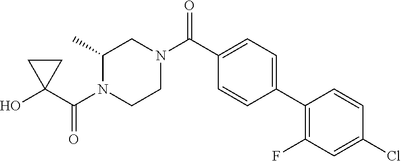 [SMILES: C[C@@H]1CN(C(=O)c2ccc(-c3ccc(Cl)cc3F)cc2)CCN1C(=O)C1(O)CC1] |
| 273 | 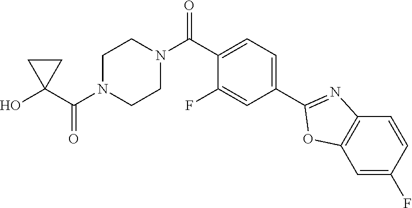 [SMILES: O=C(c1ccc(-c2nc3ccc(F)cc3o2)cc1F)N1CCN(C(=O)C2(O)CC2)CC1] |
| 274 | 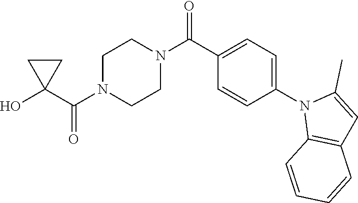 [SMILES: Cc1cc2ccccc2n1-c1ccc(C(=O)N2CCN(C(=O)C3(O)CC3)CC2)cc1] |
| 275 | 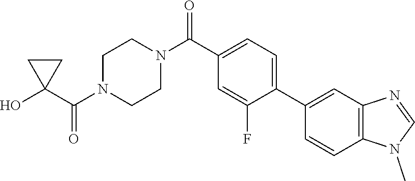 [SMILES: Cn1cnc2cc(-c3ccc(C(=O)N4CCN(C(=O)C5(O)CC5)CC4)cc3F)ccc21] |
| 276 | 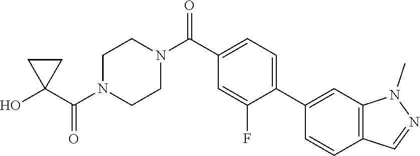 [SMILES: Cn1ncc2ccc(-c3ccc(C(=O)N4CCN(C(=O)C5(O)CC5)CC4)cc3F)cc21] |
| 277 | 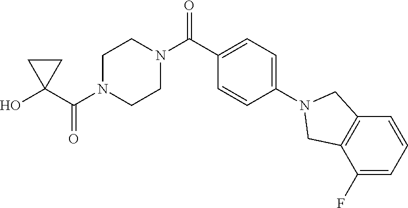 [SMILES: O=C(c1ccc(N2Cc3cccc(F)c3C2)cc1)N1CCN(C(=O)C2(O)CC2)CC1] |
| 278 | 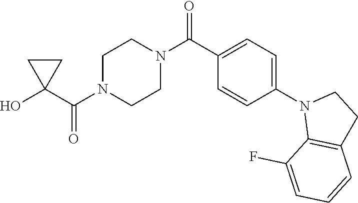 [SMILES: O=C(c1ccc(N2CCc3cccc(F)c32)cc1)N1CCN(C(=O)C2(O)CC2)CC1] |
| 279 | 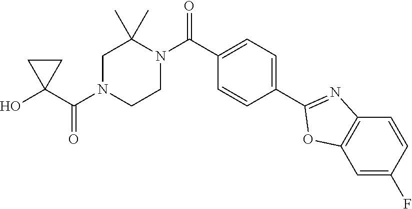 [SMILES: CC1(C)CN(C(=O)C2(O)CC2)CCN1C(=O)c1ccc(-c2nc3ccc(F)cc3o2)cc1] |
| 280 | 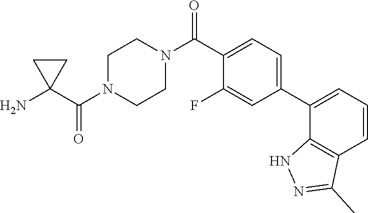 [SMILES: Cc1n[nH]c2c(-c3ccc(C(=O)N4CCN(C(=O)C5(N)CC5)CC4)c(F)c3)cccc12] |
| 281 | 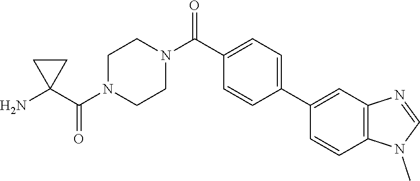 [SMILES: Cn1cnc2cc(-c3ccc(C(=O)N4CCN(C(=O)C5(N)CC5)CC4)cc3)ccc21] |
| 282 | 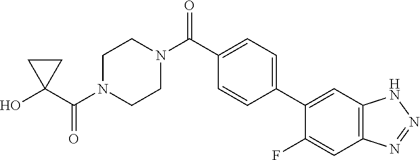 [SMILES: O=C(c1ccc(-c2cc3[nH]nnc3cc2F)cc1)N1CCN(C(=O)C2(O)CC2)CC1] |
| 283 | 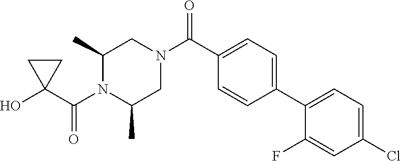 [SMILES: C[C@@H]1CN(C(=O)c2ccc(-c3ccc(Cl)cc3F)cc2)C[C@H](C)N1C(=O)C1(O)CC1] |
| 284 | 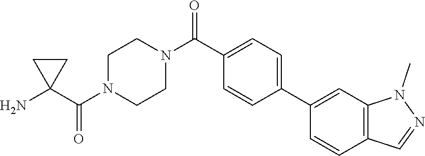 [SMILES: Cn1ncc2ccc(-c3ccc(C(=O)N4CCN(C(=O)C5(N)CC5)CC4)cc3)cc21] |
| 285 | 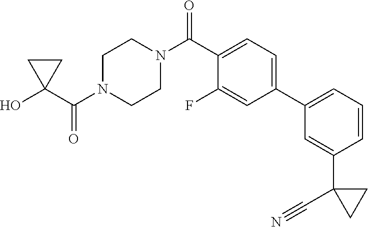 [SMILES: N#CC1(c2cccc(-c3ccc(C(=O)N4CCN(C(=O)C5(O)CC5)CC4)c(F)c3)c2)CC1] |
| 286 | 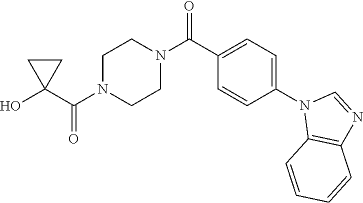 [SMILES: O=C(c1ccc(-n2cnc3ccccc32)cc1)N1CCN(C(=O)C2(O)CC2)CC1] |
| 287 | 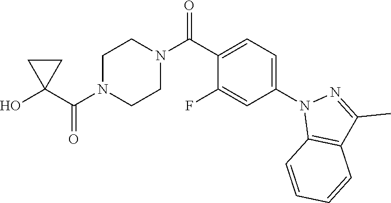 [SMILES: Cc1nn(-c2ccc(C(=O)N3CCN(C(=O)C4(O)CC4)CC3)c(F)c2)c2ccccc12] |
| 288 | 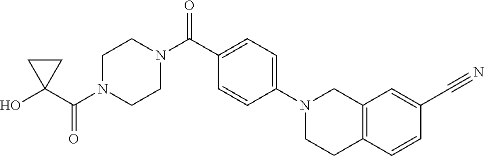 [SMILES: N#Cc1ccc2c(c1)CN(c1ccc(C(=O)N3CCN(C(=O)C4(O)CC4)CC3)cc1)CC2] |
| 289 | 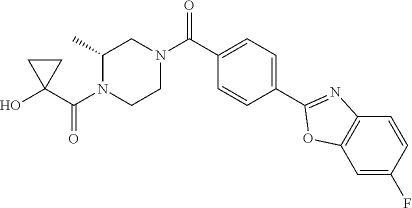 [SMILES: C[C@@H]1CN(C(=O)c2ccc(-c3nc4ccc(F)cc4o3)cc2)CCN1C(=O)C1(O)CC1] |
| 290 | 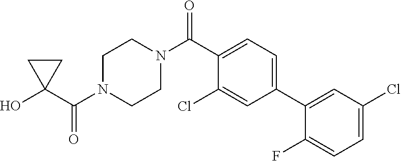 [SMILES: O=C(c1ccc(-c2cc(Cl)ccc2F)cc1Cl)N1CCN(C(=O)C2(O)CC2)CC1] |
| 291 | 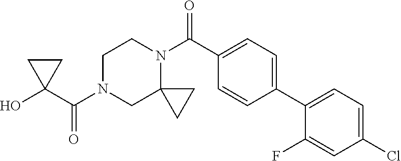 [SMILES: O=C(c1ccc(-c2ccc(Cl)cc2F)cc1)N1CCN(C(=O)C2(O)CC2)CC12CC2] |
| 292 | 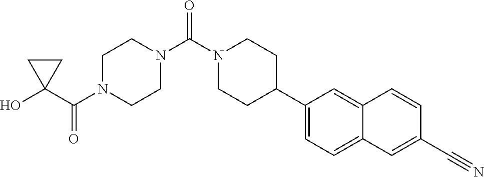 [SMILES: N#Cc1ccc2cc(C3CCN(C(=O)N4CCN(C(=O)C5(O)CC5)CC4)CC3)ccc2c1] |
| 293 | 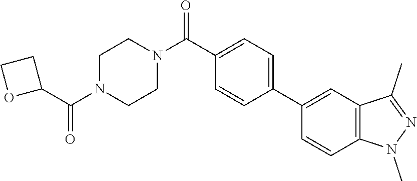 [SMILES: Cc1nn(C)c2ccc(-c3ccc(C(=O)N4CCN(C(=O)C5CCO5)CC4)cc3)cc12] |
| 294 | 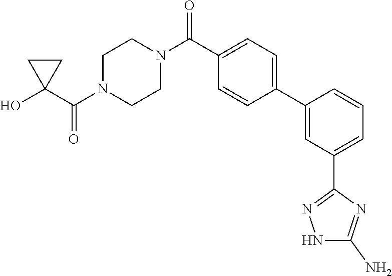 [SMILES: Nc1nc(-c2cccc(-c3ccc(C(=O)N4CCN(C(=O)C5(O)CC5)CC4)cc3)c2)n[nH]1] |
| 295 | 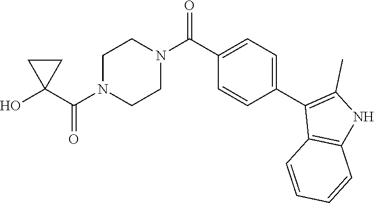 [SMILES: Cc1[nH]c2ccccc2c1-c1ccc(C(=O)N2CCN(C(=O)C3(O)CC3)CC2)cc1] |
| 296 | 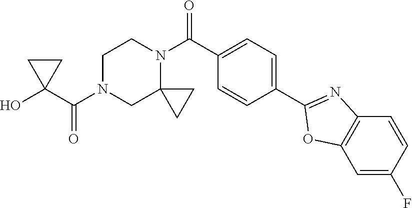 [SMILES: O=C(c1ccc(-c2nc3ccc(F)cc3o2)cc1)N1CCN(C(=O)C2(O)CC2)CC12CC2] |
| 297 | 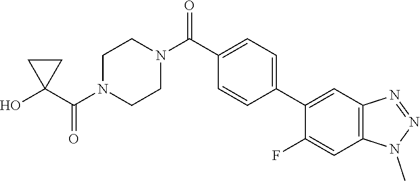 [SMILES: Cn1nnc2cc(-c3ccc(C(=O)N4CCN(C(=O)C5(O)CC5)CC4)cc3)c(F)cc21] |
| 298 | 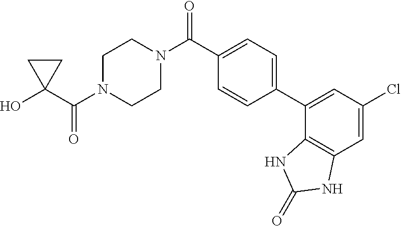 [SMILES: O=C(c1ccc(-c2cc(Cl)cc3[nH]c(=O)[nH]c23)cc1)N1CCN(C(=O)C2(O)CC2)CC1] |
| 299 | 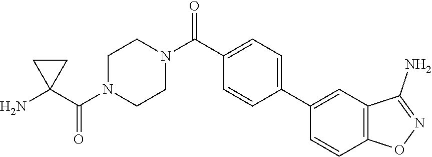 [SMILES: Nc1noc2ccc(-c3ccc(C(=O)N4CCN(C(=O)C5(N)CC5)CC4)cc3)cc12] |
| 300 | 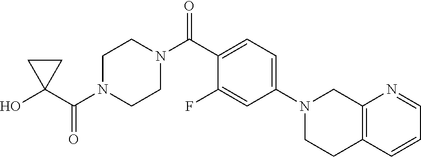 [SMILES: O=C(c1ccc(N2CCc3cccnc3C2)cc1F)N1CCN(C(=O)C2(O)CC2)CC1] |
| 301 | 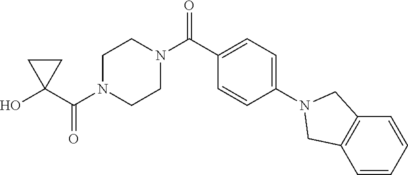 [SMILES: O=C(c1ccc(N2Cc3ccccc3C2)cc1)N1CCN(C(=O)C2(O)CC2)CC1] |
| 302 | 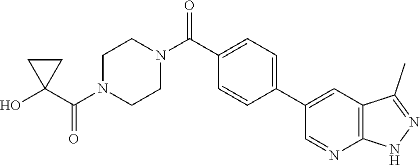 [SMILES: Cc1n[nH]c2ncc(-c3ccc(C(=O)N4CCN(C(=O)C5(O)CC5)CC4)cc3)cc12] |
| 303 | 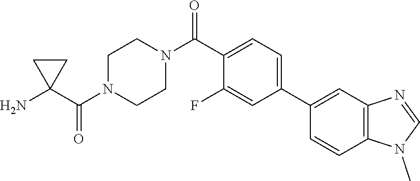 [SMILES: Cn1cnc2cc(-c3ccc(C(=O)N4CCN(C(=O)C5(N)CC5)CC4)c(F)c3)ccc21] |
| 304 | 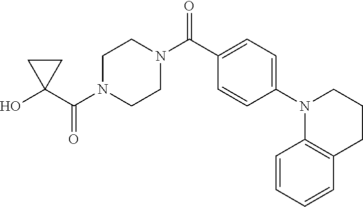 [SMILES: O=C(c1ccc(N2CCCc3ccccc32)cc1)N1CCN(C(=O)C2(O)CC2)CC1] |
| 305 | 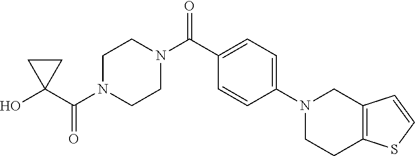 [SMILES: O=C(c1ccc(N2CCc3sccc3C2)cc1)N1CCN(C(=O)C2(O)CC2)CC1] |
| 306 | 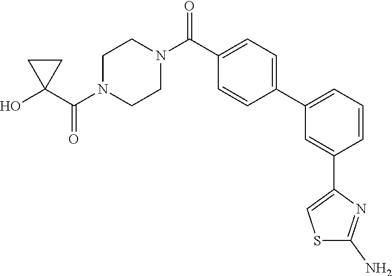 [SMILES: Nc1nc(-c2cccc(-c3ccc(C(=O)N4CCN(C(=O)C5(O)CC5)CC4)cc3)c2)cs1] |
| 307 | 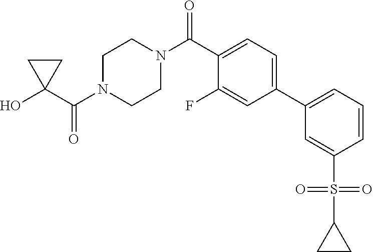 [SMILES: O=C(c1ccc(-c2cccc(S(=O)(=O)C3CC3)c2)cc1F)N1CCN(C(=O)C2(O)CC2)CC1] |
| 308 | 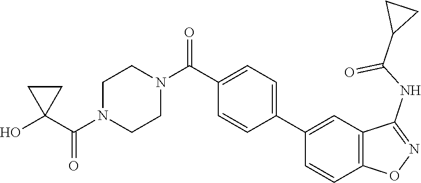 [SMILES: O=C(Nc1noc2ccc(-c3ccc(C(=O)N4CCN(C(=O)C5(O)CC5)CC4)cc3)cc12)C1CC1] |
| 309 | 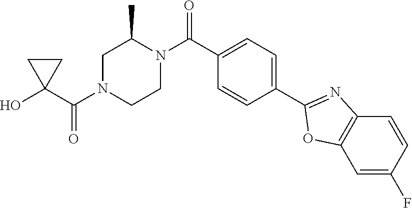 [SMILES: C[C@@H]1CN(C(=O)C2(O)CC2)CCN1C(=O)c1ccc(-c2nc3ccc(F)cc3o2)cc1] |
| 310 | 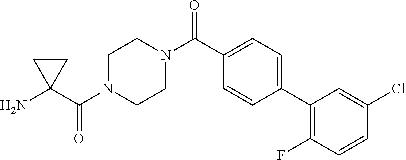 [SMILES: NC1(C(=O)N2CCN(C(=O)c3ccc(-c4cc(Cl)ccc4F)cc3)CC2)CC1] |
| 311 | 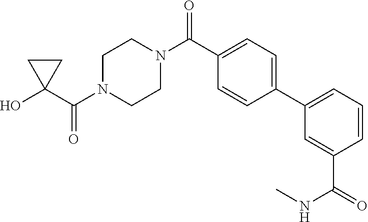 [SMILES: CNC(=O)c1cccc(-c2ccc(C(=O)N3CCN(C(=O)C4(O)CC4)CC3)cc2)c1] |
| 312 | 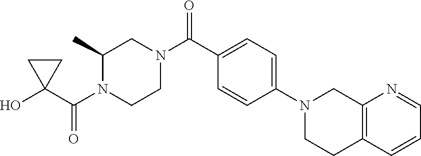 [SMILES: C[C@H]1CN(C(=O)c2ccc(N3CCc4cccnc4C3)cc2)CCN1C(=O)C1(O)CC1] |
| 313 | 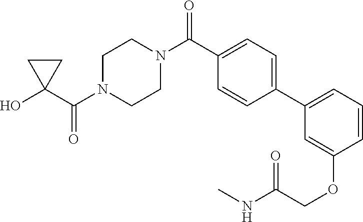 [SMILES: CNC(=O)COc1cccc(-c2ccc(C(=O)N3CCN(C(=O)C4(O)CC4)CC3)cc2)c1] |
| 314 | 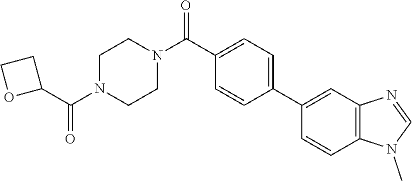 [SMILES: Cn1cnc2cc(-c3ccc(C(=O)N4CCN(C(=O)C5CCO5)CC4)cc3)ccc21] |
| 315 | 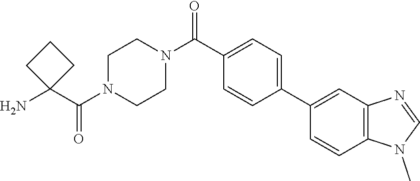 [SMILES: Cn1cnc2cc(-c3ccc(C(=O)N4CCN(C(=O)C5(N)CCC5)CC4)cc3)ccc21] |
| 316 | 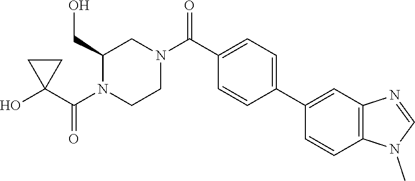 [SMILES: Cn1cnc2cc(-c3ccc(C(=O)N4CCN(C(=O)C5(O)CC5)[C@@H](CO)C4)cc3)ccc21] |
| 317 | 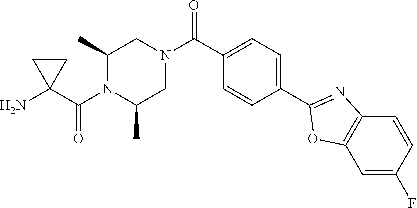 [SMILES: C[C@@H]1CN(C(=O)c2ccc(-c3nc4ccc(F)cc4o3)cc2)C[C@H](C)N1C(=O)C1(N)CC1] |
| 318 | 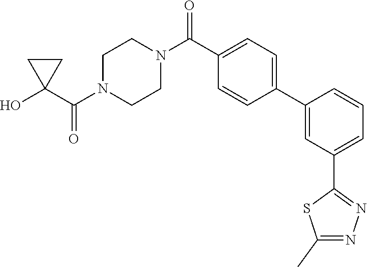 [SMILES: Cc1nnc(-c2cccc(-c3ccc(C(=O)N4CCN(C(=O)C5(O)CC5)CC4)cc3)c2)s1] |
| 319 | 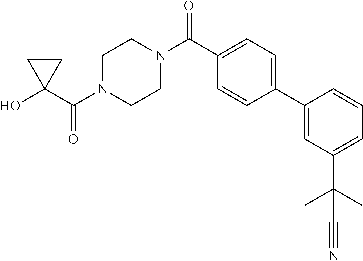 [SMILES: CC(C)(C#N)c1cccc(-c2ccc(C(=O)N3CCN(C(=O)C4(O)CC4)CC3)cc2)c1] |
| 320 | 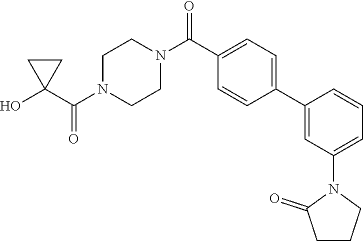 [SMILES: O=C(c1ccc(-c2cccc(N3CCCC3=O)c2)cc1)N1CCN(C(=O)C2(O)CC2)CC1] |
| 321 | 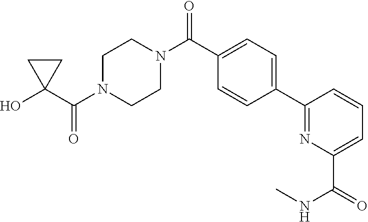 [SMILES: CNC(=O)c1cccc(-c2ccc(C(=O)N3CCN(C(=O)C4(O)CC4)CC3)cc2)n1] |
| 322 | 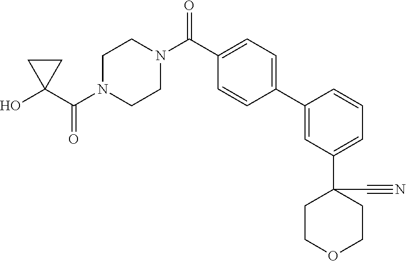 [SMILES: N#CC1(c2cccc(-c3ccc(C(=O)N4CCN(C(=O)C5(O)CC5)CC4)cc3)c2)CCOCC1] |
| 323 | 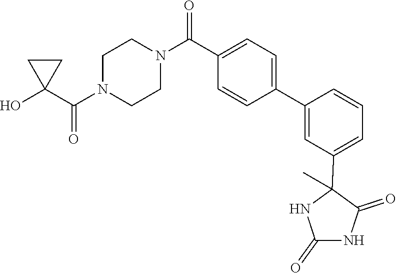 [SMILES: CC1(c2cccc(-c3ccc(C(=O)N4CCN(C(=O)C5(O)CC5)CC4)cc3)c2)NC(=O)NC1=O] |
| 324 | 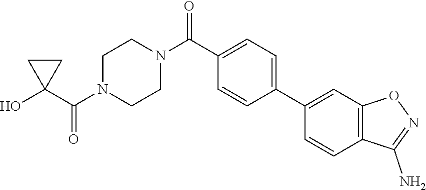 [SMILES: Nc1noc2cc(-c3ccc(C(=O)N4CCN(C(=O)C5(O)CC5)CC4)cc3)ccc12] |
| 325 | 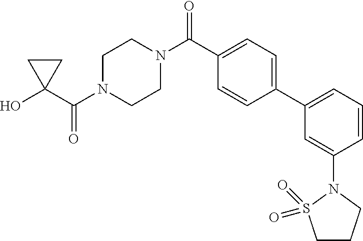 [SMILES: O=C(c1ccc(-c2cccc(N3CCCS3(=O)=O)c2)cc1)N1CCN(C(=O)C2(O)CC2)CC1] |
| 326 | 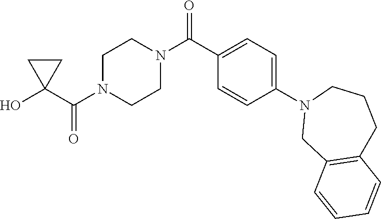 [SMILES: O=C(c1ccc(N2CCCc3ccccc3C2)cc1)N1CCN(C(=O)C2(O)CC2)CC1] |
| 327 | 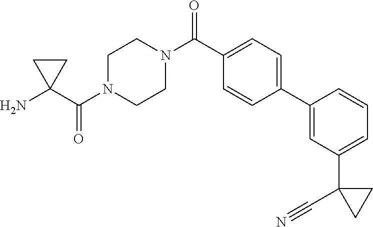 [SMILES: N#CC1(c2cccc(-c3ccc(C(=O)N4CCN(C(=O)C5(N)CC5)CC4)cc3)c2)CC1] |
| 328 | 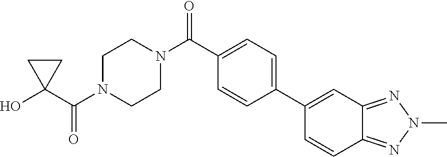 [SMILES: Cn1nc2ccc(-c3ccc(C(=O)N4CCN(C(=O)C5(O)CC5)CC4)cc3)cc2n1] |
| 329 | 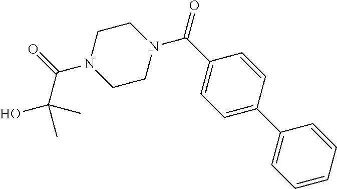 [SMILES: CC(C)(O)C(=O)N1CCN(C(=O)c2ccc(-c3ccccc3)cc2)CC1] |
| 330 | 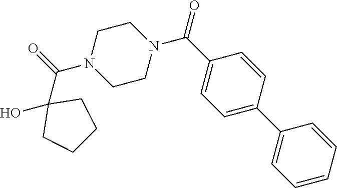 [SMILES: O=C(c1ccc(-c2ccccc2)cc1)N1CCN(C(=O)C2(O)CCCC2)CC1] |
| 331 | 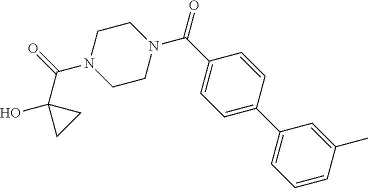 [SMILES: Cc1cccc(-c2ccc(C(=O)N3CCN(C(=O)C4(O)CC4)CC3)cc2)c1] |
| 332 | 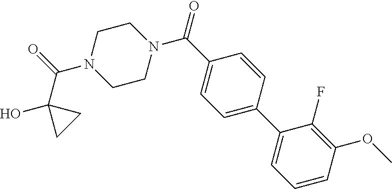 [SMILES: COc1cccc(-c2ccc(C(=O)N3CCN(C(=O)C4(O)CC4)CC3)cc2)c1F] |
| 333 | 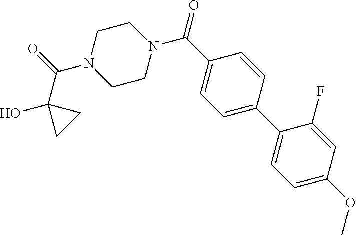 [SMILES: COc1ccc(-c2ccc(C(=O)N3CCN(C(=O)C4(O)CC4)CC3)cc2)c(F)c1] |
| 334 | 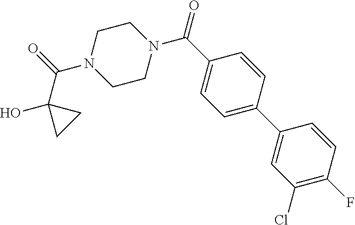 [SMILES: O=C(c1ccc(-c2ccc(F)c(Cl)c2)cc1)N1CCN(C(=O)C2(O)CC2)CC1] |
| 335 | 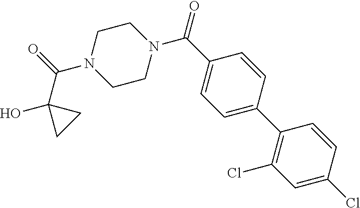 [SMILES: O=C(c1ccc(-c2ccc(Cl)cc2Cl)cc1)N1CCN(C(=O)C2(O)CC2)CC1] |
| 336 | 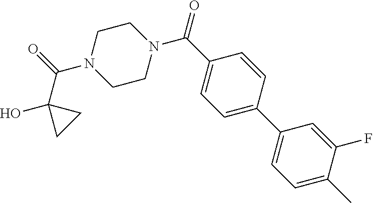 [SMILES: Cc1ccc(-c2ccc(C(=O)N3CCN(C(=O)C4(O)CC4)CC3)cc2)cc1F] |
| 337 | 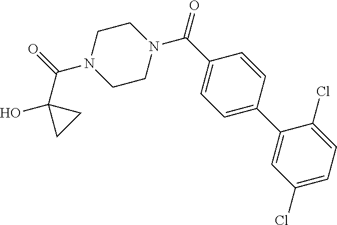 [SMILES: O=C(c1ccc(-c2cc(Cl)ccc2Cl)cc1)N1CCN(C(=O)C2(O)CC2)CC1] |
| 338 | 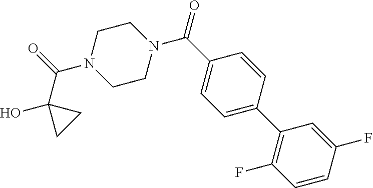 [SMILES: O=C(c1ccc(-c2cc(F)ccc2F)cc1)N1CCN(C(=O)C2(O)CC2)CC1] |
| 339 | 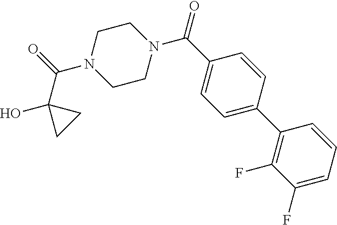 [SMILES: O=C(c1ccc(-c2cccc(F)c2F)cc1)N1CCN(C(=O)C2(O)CC2)CC1] |
| 340 | 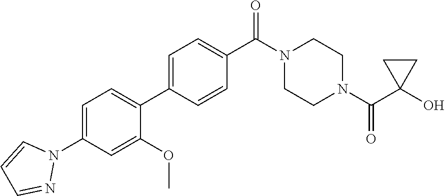 [SMILES: COc1cc(-n2cccn2)ccc1-c1ccc(C(=O)N2CCN(C(=O)C3(O)CC3)CC2)cc1] |
| 341 | 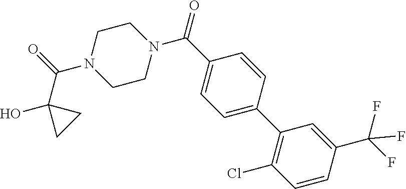 [SMILES: O=C(c1ccc(-c2cc(C(F)(F)F)ccc2Cl)cc1)N1CCN(C(=O)C2(O)CC2)CC1] |
| 342 | 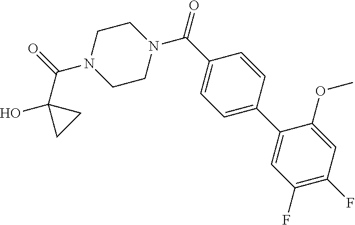 [SMILES: COc1cc(F)c(F)cc1-c1ccc(C(=O)N2CCN(C(=O)C3(O)CC3)CC2)cc1] |
| 343 | 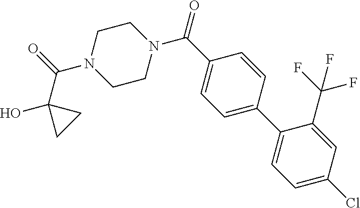 [SMILES: O=C(c1ccc(-c2ccc(Cl)cc2C(F)(F)F)cc1)N1CCN(C(=O)C2(O)CC2)CC1] |
| 344 | 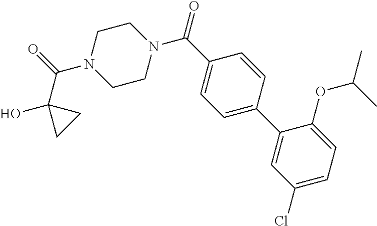 [SMILES: CC(C)Oc1ccc(Cl)cc1-c1ccc(C(=O)N2CCN(C(=O)C3(O)CC3)CC2)cc1] |
| 345 | 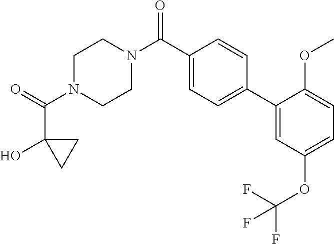 [SMILES: COc1ccc(OC(F)(F)F)cc1-c1ccc(C(=O)N2CCN(C(=O)C3(O)CC3)CC2)cc1] |
| 346 | 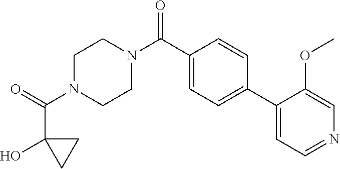 [SMILES: COc1cnccc1-c1ccc(C(=O)N2CCN(C(=O)C3(O)CC3)CC2)cc1] |
| 347 | 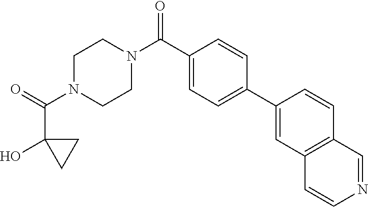 [SMILES: O=C(c1ccc(-c2ccc3cnccc3c2)cc1)N1CCN(C(=O)C2(O)CC2)CC1] |
| 348 | 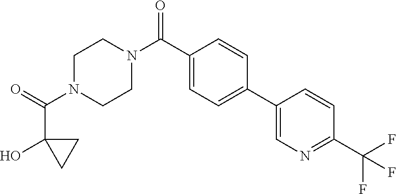 [SMILES: O=C(c1ccc(-c2ccc(C(F)(F)F)nc2)cc1)N1CCN(C(=O)C2(O)CC2)CC1] |
| 349 | 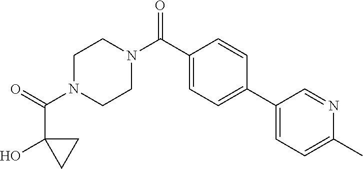 [SMILES: Cc1ccc(-c2ccc(C(=O)N3CCN(C(=O)C4(O)CC4)CC3)cc2)cn1] |
| 350 | 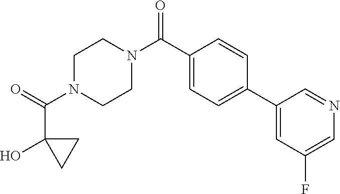 [SMILES: O=C(c1ccc(-c2cncc(F)c2)cc1)N1CCN(C(=O)C2(O)CC2)CC1] |
| 351 | 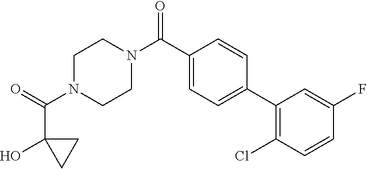 [SMILES: O=C(c1ccc(-c2cc(F)ccc2Cl)cc1)N1CCN(C(=O)C2(O)CC2)CC1] |
| 352 | 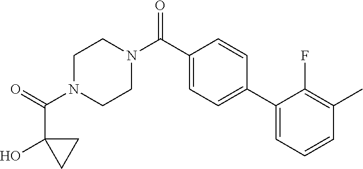 [SMILES: Cc1cccc(-c2ccc(C(=O)N3CCN(C(=O)C4(O)CC4)CC3)cc2)c1F] |
| 353 | 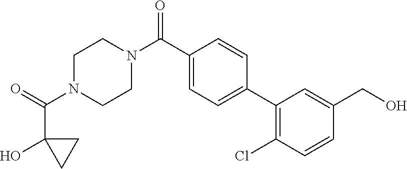 [SMILES: O=C(c1ccc(-c2cc(CO)ccc2Cl)cc1)N1CCN(C(=O)C2(O)CC2)CC1] |
| 354 | 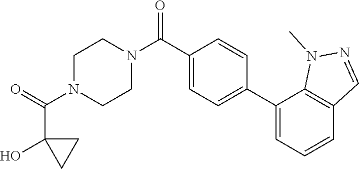 [SMILES: Cn1ncc2cccc(-c3ccc(C(=O)N4CCN(C(=O)C5(O)CC5)CC4)cc3)c21] |
| 355 | 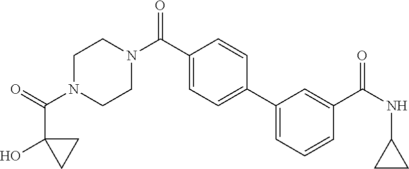 [SMILES: O=C(NC1CC1)c1cccc(-c2ccc(C(=O)N3CCN(C(=O)C4(O)CC4)CC3)cc2)c1] |
| 356 | 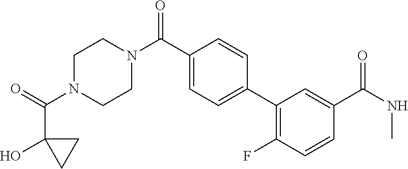 [SMILES: CNC(=O)c1ccc(F)c(-c2ccc(C(=O)N3CCN(C(=O)C4(O)CC4)CC3)cc2)c1] |
| 357 | 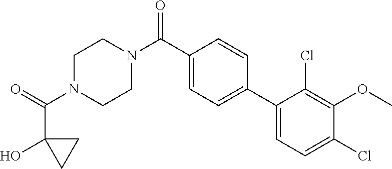 [SMILES: COc1c(Cl)ccc(-c2ccc(C(=O)N3CCN(C(=O)C4(O)CC4)CC3)cc2)c1Cl] |
| 358 | 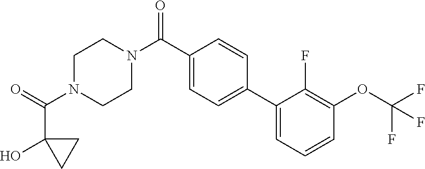 [SMILES: O=C(c1ccc(-c2cccc(OC(F)(F)F)c2F)cc1)N1CCN(C(=O)C2(O)CC2)CC1] |
| 359 | 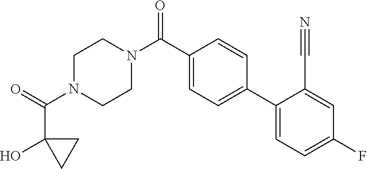 [SMILES: N#Cc1cc(F)ccc1-c1ccc(C(=O)N2CCN(C(=O)C3(O)CC3)CC2)cc1] |
| 360 | 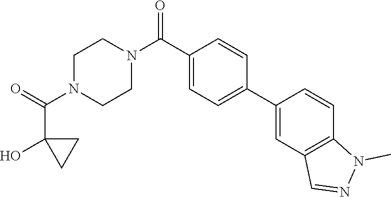 [SMILES: Cn1ncc2cc(-c3ccc(C(=O)N4CCN(C(=O)C5(O)CC5)CC4)cc3)ccc21] |
| 361 | 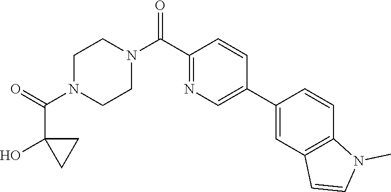 [SMILES: Cn1ccc2cc(-c3ccc(C(=O)N4CCN(C(=O)C5(O)CC5)CC4)nc3)ccc21] |
| 362 | 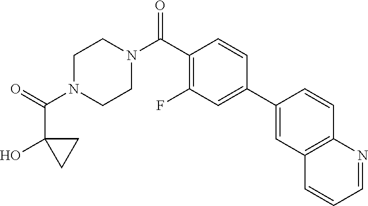 [SMILES: O=C(c1ccc(-c2ccc3ncccc3c2)cc1F)N1CCN(C(=O)C2(O)CC2)CC1] |
| 363 | 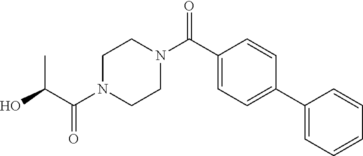 [SMILES: C[C@H](O)C(=O)N1CCN(C(=O)c2ccc(-c3ccccc3)cc2)CC1] |
| 364 | 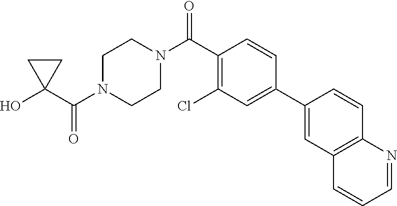 [SMILES: O=C(c1ccc(-c2ccc3ncccc3c2)cc1Cl)N1CCN(C(=O)C2(O)CC2)CC1] |
| 365 | 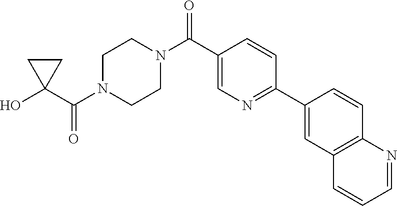 [SMILES: O=C(c1ccc(-c2ccc3ncccc3c2)nc1)N1CCN(C(=O)C2(O)CC2)CC1] |
| 366 | 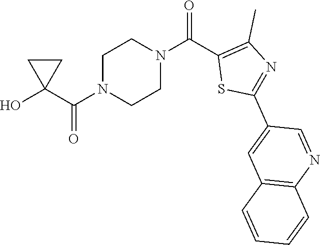 [SMILES: Cc1nc(-c2cnc3ccccc3c2)sc1C(=O)N1CCN(C(=O)C2(O)CC2)CC1] |
| 367 | 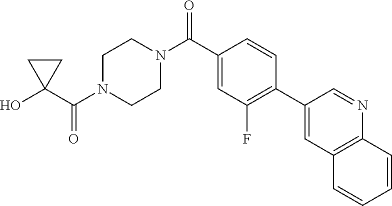 [SMILES: O=C(c1ccc(-c2cnc3ccccc3c2)c(F)c1)N1CCN(C(=O)C2(O)CC2)CC1] |
| 368 | 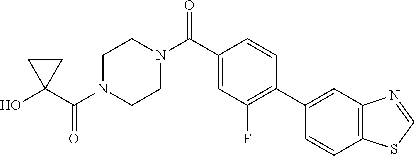 [SMILES: O=C(c1ccc(-c2ccc3scnc3c2)c(F)c1)N1CCN(C(=O)C2(O)CC2)CC1] |
| 369 | 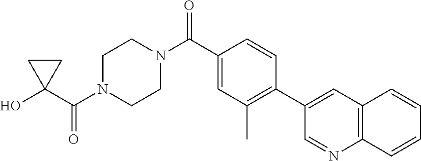 [SMILES: Cc1cc(C(=O)N2CCN(C(=O)C3(O)CC3)CC2)ccc1-c1cnc2ccccc2c1] |
| 370 | 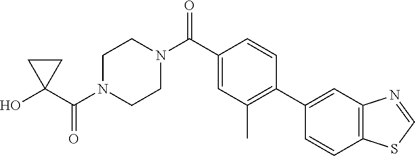 [SMILES: Cc1cc(C(=O)N2CCN(C(=O)C3(O)CC3)CC2)ccc1-c1ccc2scnc2c1] |
| 371 | 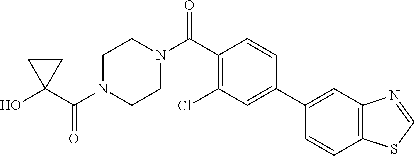 [SMILES: O=C(c1ccc(-c2ccc3scnc3c2)cc1Cl)N1CCN(C(=O)C2(O)CC2)CC1] |
| 372 | 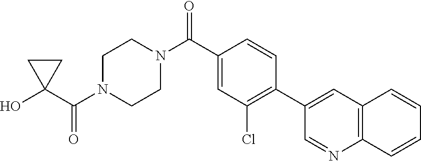 [SMILES: O=C(c1ccc(-c2cnc3ccccc3c2)c(Cl)c1)N1CCN(C(=O)C2(O)CC2)CC1] |
| 373 | 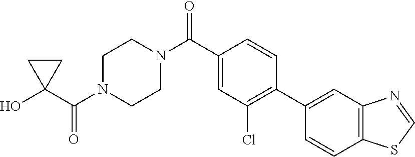 [SMILES: O=C(c1ccc(-c2ccc3scnc3c2)c(Cl)c1)N1CCN(C(=O)C2(O)CC2)CC1] |
| 374 | 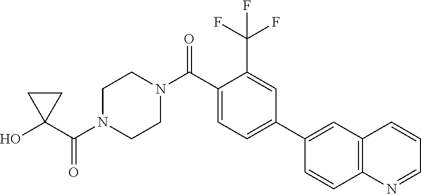 [SMILES: O=C(c1ccc(-c2ccc3ncccc3c2)cc1C(F)(F)F)N1CCN(C(=O)C2(O)CC2)CC1] |
| 375 | 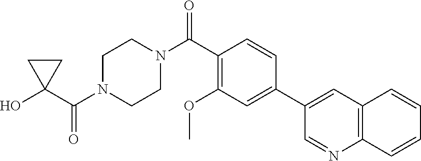 [SMILES: COc1cc(-c2cnc3ccccc3c2)ccc1C(=O)N1CCN(C(=O)C2(O)CC2)CC1] |
| 376 | 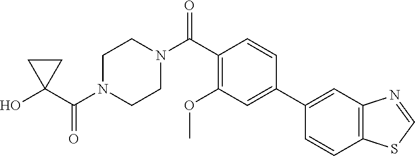 [SMILES: COc1cc(-c2ccc3scnc3c2)ccc1C(=O)N1CCN(C(=O)C2(O)CC2)CC1] |
| 377 | 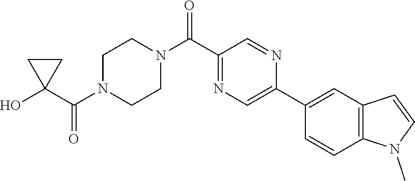 [SMILES: Cn1ccc2cc(-c3cnc(C(=O)N4CCN(C(=O)C5(O)CC5)CC4)cn3)ccc21] |
| 378 | 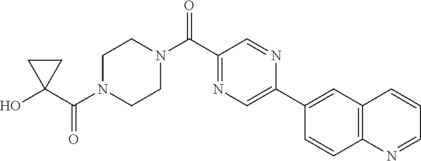 [SMILES: O=C(c1cnc(-c2ccc3ncccc3c2)cn1)N1CCN(C(=O)C2(O)CC2)CC1] |
| 379 | 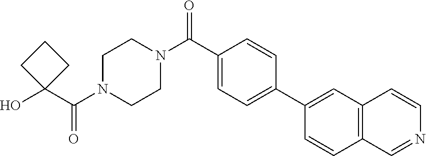 [SMILES: O=C(c1ccc(-c2ccc3cnccc3c2)cc1)N1CCN(C(=O)C2(O)CCC2)CC1] |
| 380 | 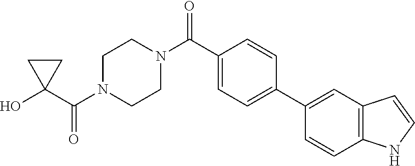 [SMILES: O=C(c1ccc(-c2ccc3[nH]ccc3c2)cc1)N1CCN(C(=O)C2(O)CC2)CC1] |
| 381 | 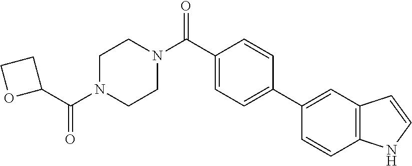 [SMILES: O=C(c1ccc(-c2ccc3[nH]ccc3c2)cc1)N1CCN(C(=O)C2CCO2)CC1] |
| 382 | 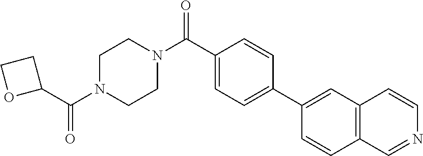 [SMILES: O=C(c1ccc(-c2ccc3cnccc3c2)cc1)N1CCN(C(=O)C2CCO2)CC1] |
| 383 | 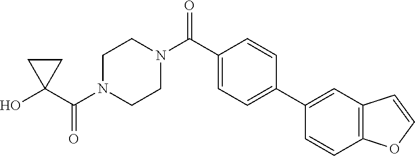 [SMILES: O=C(c1ccc(-c2ccc3occc3c2)cc1)N1CCN(C(=O)C2(O)CC2)CC1] |
| 384 | 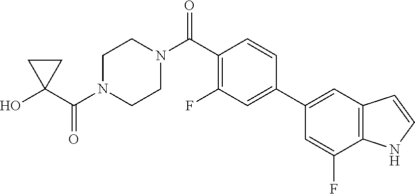 [SMILES: O=C(c1ccc(-c2cc(F)c3[nH]ccc3c2)cc1F)N1CCN(C(=O)C2(O)CC2)CC1] |
| 385 | 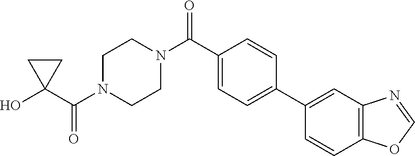 [SMILES: O=C(c1ccc(-c2ccc3ocnc3c2)cc1)N1CCN(C(=O)C2(O)CC2)CC1] |
| 386 | 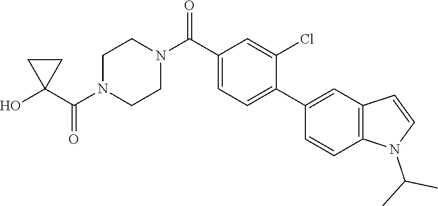 [SMILES: CC(C)n1ccc2cc(-c3ccc(C(=O)N4CCN(C(=O)C5(O)CC5)CC4)cc3Cl)ccc21] |
| 387 | 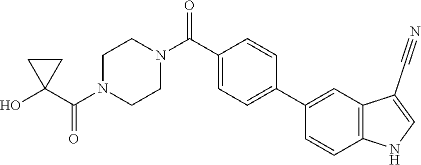 [SMILES: N#Cc1c[nH]c2ccc(-c3ccc(C(=O)N4CCN(C(=O)C5(O)CC5)CC4)cc3)cc12] |
| 388 | 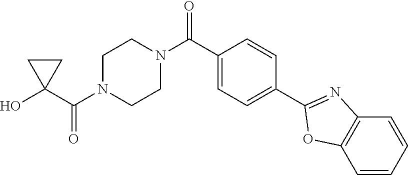 [SMILES: O=C(c1ccc(-c2nc3ccccc3o2)cc1)N1CCN(C(=O)C2(O)CC2)CC1] |
| 389 | 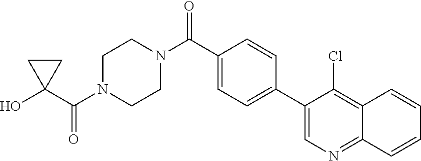 [SMILES: O=C(c1ccc(-c2cnc3ccccc3c2Cl)cc1)N1CCN(C(=O)C2(O)CC2)CC1] |
| 390 | 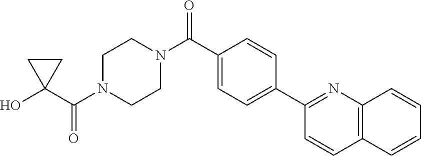 [SMILES: O=C(c1ccc(-c2ccc3ccccc3n2)cc1)N1CCN(C(=O)C2(O)CC2)CC1] |
| 391 | 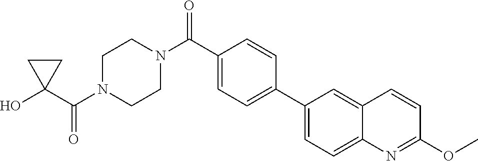 [SMILES: COc1ccc2cc(-c3ccc(C(=O)N4CCN(C(=O)C5(O)CC5)CC4)cc3)ccc2n1] |
| 392 | 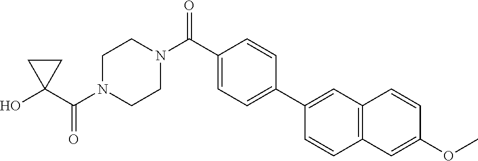 [SMILES: COc1ccc2cc(-c3ccc(C(=O)N4CCN(C(=O)C5(O)CC5)CC4)cc3)ccc2c1] |
| 393 | 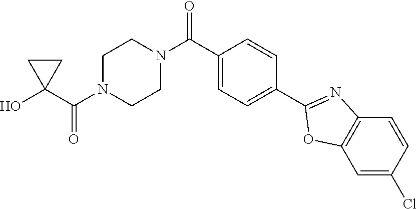 [SMILES: O=C(c1ccc(-c2nc3ccc(Cl)cc3o2)cc1)N1CCN(C(=O)C2(O)CC2)CC1] |
| 394 | 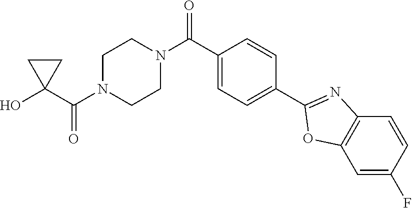 [SMILES: O=C(c1ccc(-c2nc3ccc(F)cc3o2)cc1)N1CCN(C(=O)C2(O)CC2)CC1] |
| 395 | 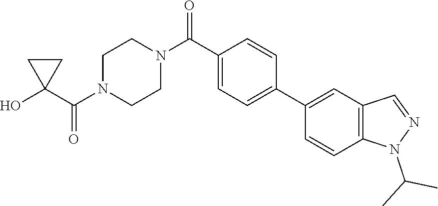 [SMILES: CC(C)n1ncc2cc(-c3ccc(C(=O)N4CCN(C(=O)C5(O)CC5)CC4)cc3)ccc21] |
| 396 | 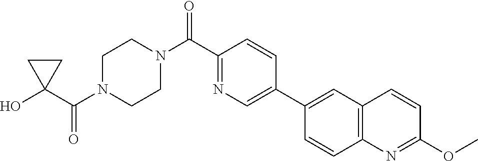 [SMILES: COc1ccc2cc(-c3ccc(C(=O)N4CCN(C(=O)C5(O)CC5)CC4)nc3)ccc2n1] |
| 397 | 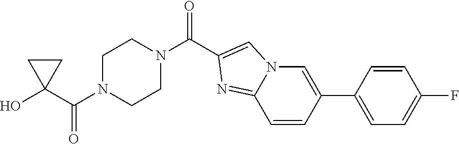 [SMILES: O=C(c1cn2cc(-c3ccc(F)cc3)ccc2n1)N1CCN(C(=O)C2(O)CC2)CC1] |
| 398 | 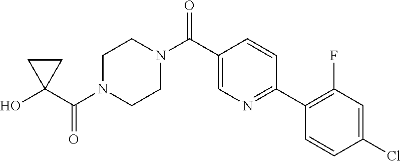 [SMILES: O=C(c1ccc(-c2ccc(Cl)cc2F)nc1)N1CCN(C(=O)C2(O)CC2)CC1] |
| 399 | 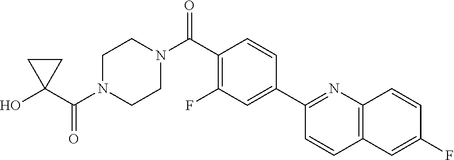 [SMILES: O=C(c1ccc(-c2ccc3cc(F)ccc3n2)cc1F)N1CCN(C(=O)C2(O)CC2)CC1] |
| 400 | 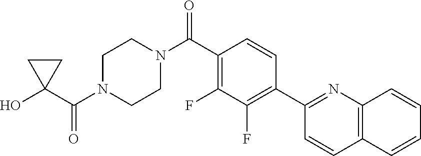 [SMILES: O=C(c1ccc(-c2ccc3ccccc3n2)c(F)c1F)N1CCN(C(=O)C2(O)CC2)CC1] |
| 401 | 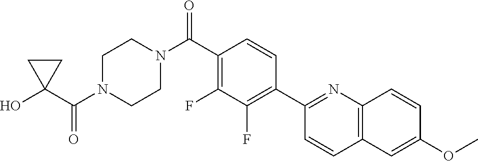 [SMILES: COc1ccc2nc(-c3ccc(C(=O)N4CCN(C(=O)C5(O)CC5)CC4)c(F)c3F)ccc2c1] |
| 402 | 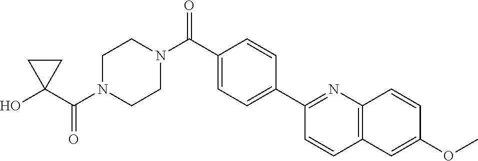 [SMILES: COc1ccc2nc(-c3ccc(C(=O)N4CCN(C(=O)C5(O)CC5)CC4)cc3)ccc2c1] |
| 403 | 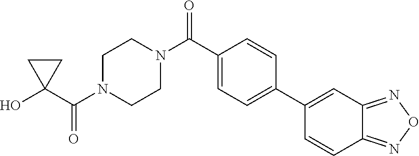 [SMILES: O=C(c1ccc(-c2ccc3nonc3c2)cc1)N1CCN(C(=O)C2(O)CC2)CC1] |
| 404 | 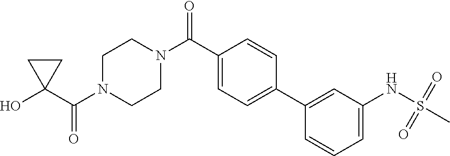 [SMILES: CS(=O)(=O)Nc1cccc(-c2ccc(C(=O)N3CCN(C(=O)C4(O)CC4)CC3)cc2)c1] |
| 405 | 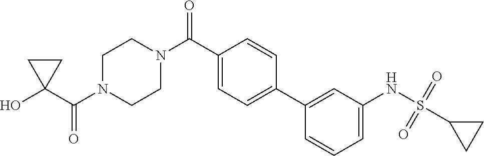 [SMILES: O=C(c1ccc(-c2cccc(NS(=O)(=O)C3CC3)c2)cc1)N1CCN(C(=O)C2(O)CC2)CC1] |
| 406 | 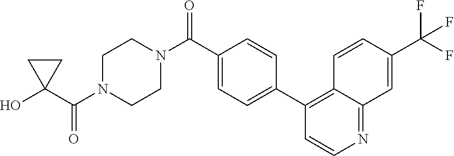 [SMILES: O=C(c1ccc(-c2ccnc3cc(C(F)(F)F)ccc23)cc1)N1CCN(C(=O)C2(O)CC2)CC1] |
| 407 | 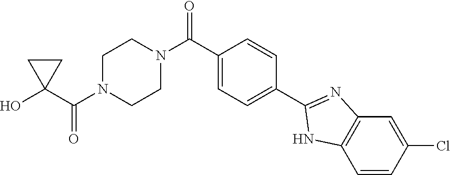 [SMILES: O=C(c1ccc(-c2nc3cc(Cl)ccc3[nH]2)cc1)N1CCN(C(=O)C2(O)CC2)CC1] |
| 408 | 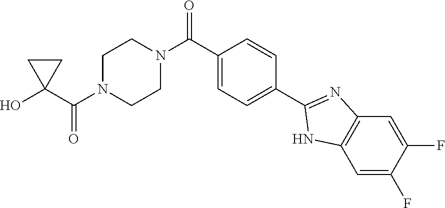 [SMILES: O=C(c1ccc(-c2nc3cc(F)c(F)cc3[nH]2)cc1)N1CCN(C(=O)C2(O)CC2)CC1] |
| 409 | 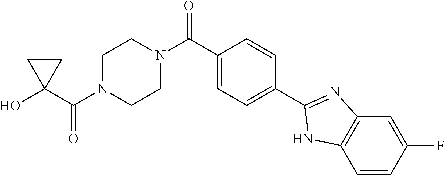 [SMILES: O=C(c1ccc(-c2nc3cc(F)ccc3[nH]2)cc1)N1CCN(C(=O)C2(O)CC2)CC1] |
| 410 | 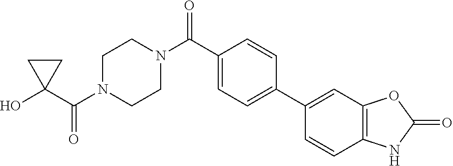 [SMILES: O=C(c1ccc(-c2ccc3[nH]c(=O)oc3c2)cc1)N1CCN(C(=O)C2(O)CC2)CC1] |
| 411 | 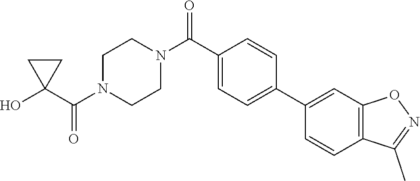 [SMILES: Cc1noc2cc(-c3ccc(C(=O)N4CCN(C(=O)C5(O)CC5)CC4)cc3)ccc12] |
| 412 | 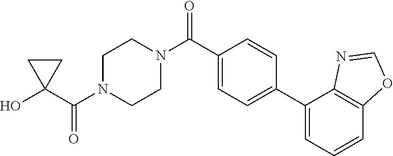 [SMILES: O=C(c1ccc(-c2cccc3ocnc23)cc1)N1CCN(C(=O)C2(O)CC2)CC1] |
| 413 | 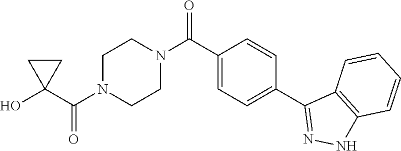 [SMILES: O=C(c1ccc(-c2n[nH]c3ccccc23)cc1)N1CCN(C(=O)C2(O)CC2)CC1] |
| 414 | 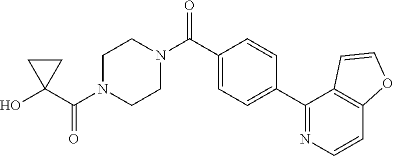 [SMILES: O=C(c1ccc(-c2nccc3occc23)cc1)N1CCN(C(=O)C2(O)CC2)CC1] |
| 415 | 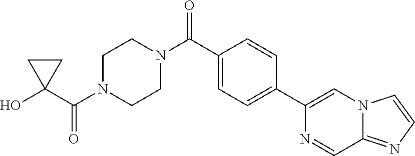 [SMILES: O=C(c1ccc(-c2cn3ccnc3cn2)cc1)N1CCN(C(=O)C2(O)CC2)CC1] |
| 416 | 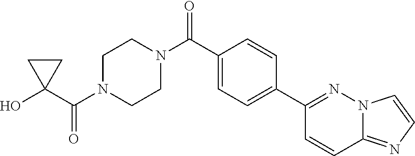 [SMILES: O=C(c1ccc(-c2ccc3nccn3n2)cc1)N1CCN(C(=O)C2(O)CC2)CC1] |
| 417 | 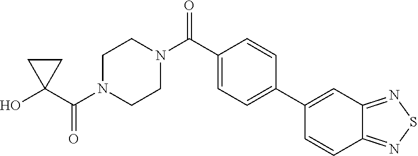 [SMILES: O=C(c1ccc(-c2ccc3nsnc3c2)cc1)N1CCN(C(=O)C2(O)CC2)CC1] |
| 418 | 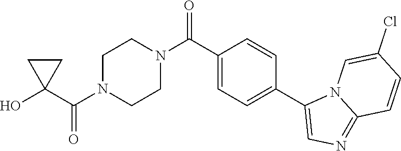 [SMILES: O=C(c1ccc(-c2cnc3ccc(Cl)cn23)cc1)N1CCN(C(=O)C2(O)CC2)CC1] |
| 419 | 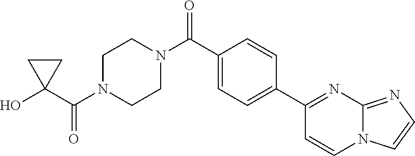 [SMILES: O=C(c1ccc(-c2ccn3ccnc3n2)cc1)N1CCN(C(=O)C2(O)CC2)CC1] |
| 420 | 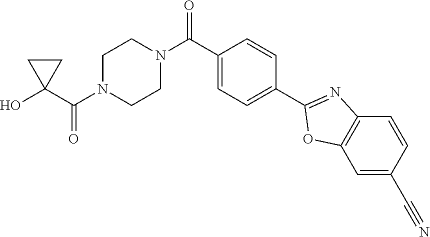 [SMILES: N#Cc1ccc2nc(-c3ccc(C(=O)N4CCN(C(=O)C5(O)CC5)CC4)cc3)oc2c1] |
| 421 | 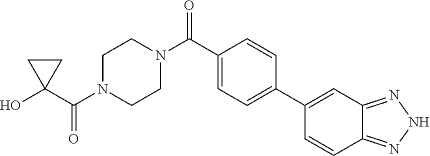 [SMILES: O=C(c1ccc(-c2ccc3n[nH]nc3c2)cc1)N1CCN(C(=O)C2(O)CC2)CC1] |
| 422 | 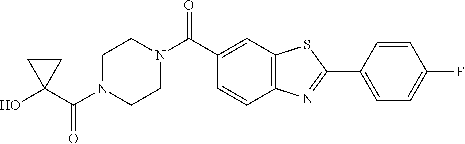 [SMILES: O=C(c1ccc2nc(-c3ccc(F)cc3)sc2c1)N1CCN(C(=O)C2(O)CC2)CC1] |
| 423 | 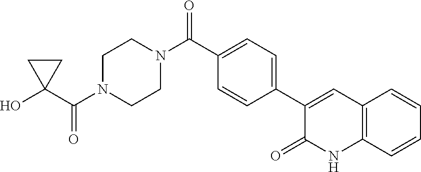 [SMILES: O=C(c1ccc(-c2cc3ccccc3[nH]c2=O)cc1)N1CCN(C(=O)C2(O)CC2)CC1] |
| 424 | 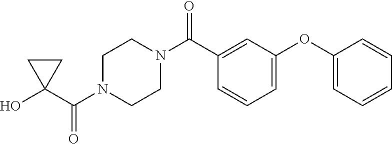 [SMILES: O=C(c1cccc(Oc2ccccc2)c1)N1CCN(C(=O)C2(O)CC2)CC1] |
| 425 | 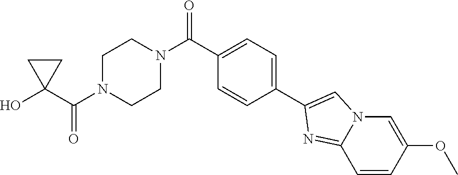 [SMILES: COc1ccc2nc(-c3ccc(C(=O)N4CCN(C(=O)C5(O)CC5)CC4)cc3)cn2c1] |
| 426 | 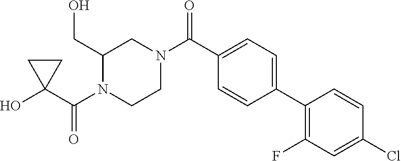 [SMILES: O=C(c1ccc(-c2ccc(Cl)cc2F)cc1)N1CCN(C(=O)C2(O)CC2)C(CO)C1] |
| 427 | 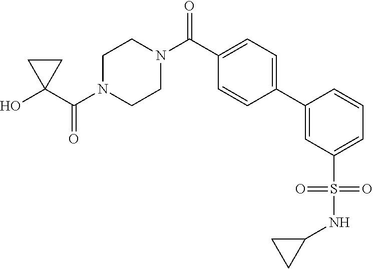 [SMILES: O=C(c1ccc(-c2cccc(S(=O)(=O)NC3CC3)c2)cc1)N1CCN(C(=O)C2(O)CC2)CC1] |
| 428 | 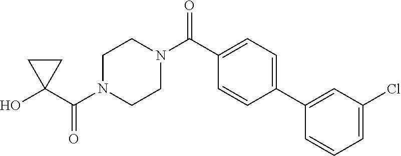 [SMILES: O=C(c1ccc(-c2cccc(Cl)c2)cc1)N1CCN(C(=O)C2(O)CC2)CC1] |
| 429 | 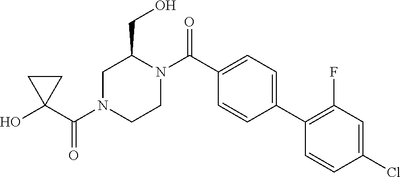 [SMILES: O=C(c1ccc(-c2ccc(Cl)cc2F)cc1)N1CCN(C(=O)C2(O)CC2)C[C@H]1CO] |
| 430 | 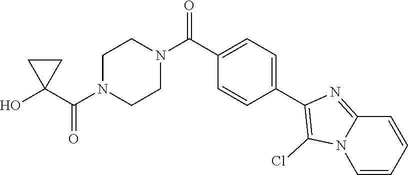 [SMILES: O=C(c1ccc(-c2nc3ccccn3c2Cl)cc1)N1CCN(C(=O)C2(O)CC2)CC1] |
| 431 | 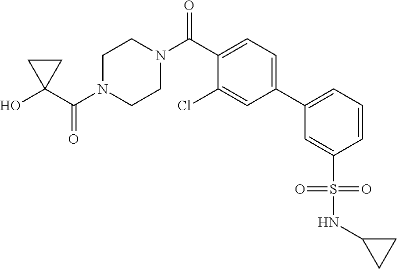 [SMILES: O=C(c1ccc(-c2cccc(S(=O)(=O)NC3CC3)c2)cc1Cl)N1CCN(C(=O)C2(O)CC2)CC1] |
| 432 | 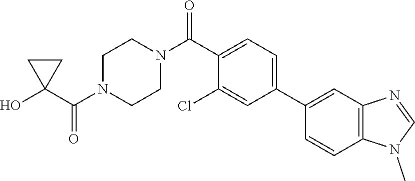 [SMILES: Cn1cnc2cc(-c3ccc(C(=O)N4CCN(C(=O)C5(O)CC5)CC4)c(Cl)c3)ccc21] |
| 433 | 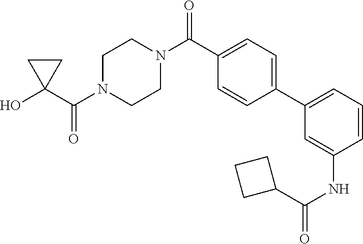 [SMILES: O=C(Nc1cccc(-c2ccc(C(=O)N3CCN(C(=O)C4(O)CC4)CC3)cc2)c1)C1CCC1] |
| 434 | 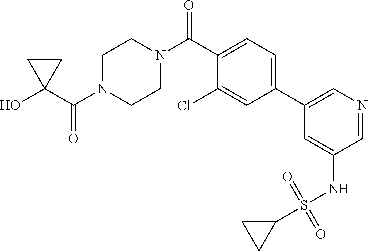 [SMILES: O=C(c1ccc(-c2cncc(NS(=O)(=O)C3CC3)c2)cc1Cl)N1CCN(C(=O)C2(O)CC2)CC1] |
| 435 | 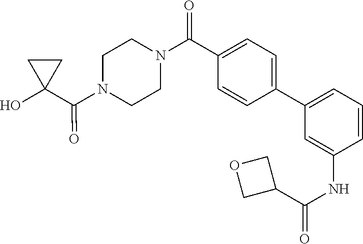 [SMILES: O=C(Nc1cccc(-c2ccc(C(=O)N3CCN(C(=O)C4(O)CC4)CC3)cc2)c1)C1COC1] |
| 436 | 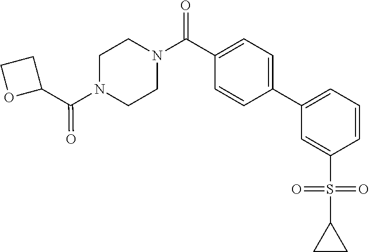 [SMILES: O=C(c1ccc(-c2cccc(S(=O)(=O)C3CC3)c2)cc1)N1CCN(C(=O)C2CCO2)CC1] |
| 437 | 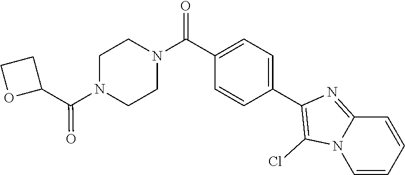 [SMILES: O=C(c1ccc(-c2nc3ccccn3c2Cl)cc1)N1CCN(C(=O)C2CCO2)CC1] |
| 438 | 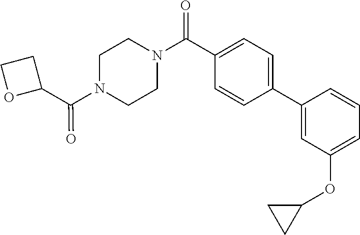 [SMILES: O=C(c1ccc(-c2cccc(OC3CC3)c2)cc1)N1CCN(C(=O)C2CCO2)CC1] |
| 439 | 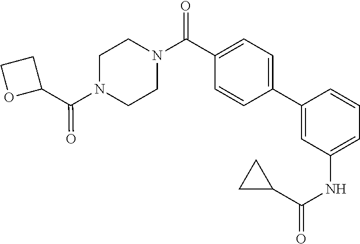 [SMILES: O=C(Nc1cccc(-c2ccc(C(=O)N3CCN(C(=O)C4CCO4)CC3)cc2)c1)C1CC1] |
| 440 | 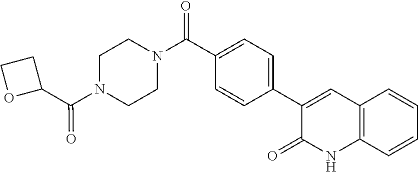 [SMILES: O=C(c1ccc(-c2cc3ccccc3[nH]c2=O)cc1)N1CCN(C(=O)C2CCO2)CC1] |
| 441 | 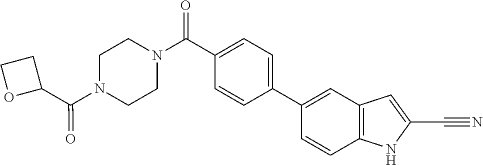 [SMILES: N#Cc1cc2cc(-c3ccc(C(=O)N4CCN(C(=O)C5CCO5)CC4)cc3)ccc2[nH]1] |
| 442 | 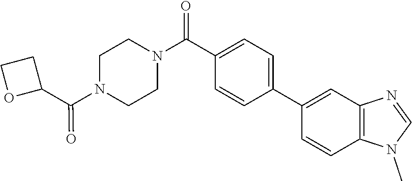 [SMILES: Cn1cnc2cc(-c3ccc(C(=O)N4CCN(C(=O)C5CCO5)CC4)cc3)ccc21] |
| 443 | 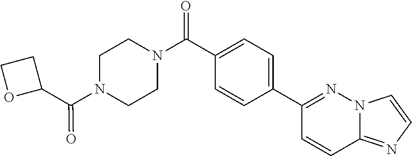 [SMILES: O=C(c1ccc(-c2ccc3nccn3n2)cc1)N1CCN(C(=O)C2CCO2)CC1] |
| 444 | 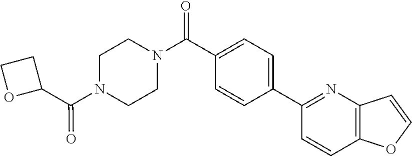 [SMILES: O=C(c1ccc(-c2ccc3occc3n2)cc1)N1CCN(C(=O)C2CCO2)CC1] |
| 445 | 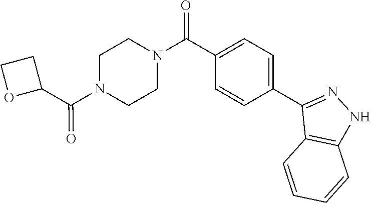 [SMILES: O=C(c1ccc(-c2n[nH]c3ccccc23)cc1)N1CCN(C(=O)C2CCO2)CC1] |
| 446 | 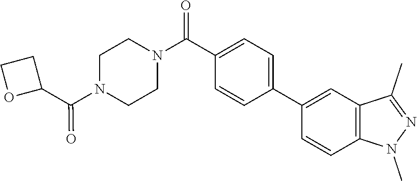 [SMILES: Cc1nn(C)c2ccc(-c3ccc(C(=O)N4CCN(C(=O)C5CCO5)CC4)cc3)cc12] |
| 447 | 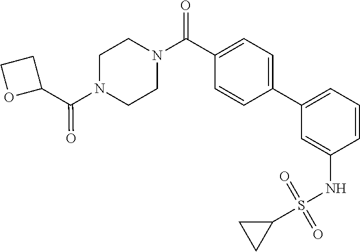 [SMILES: O=C(c1ccc(-c2cccc(NS(=O)(=O)C3CC3)c2)cc1)N1CCN(C(=O)C2CCO2)CC1] |
| 448 | 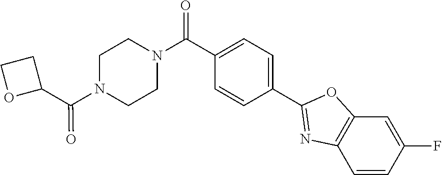 [SMILES: O=C(c1ccc(-c2nc3ccc(F)cc3o2)cc1)N1CCN(C(=O)C2CCO2)CC1] |
| 449 | 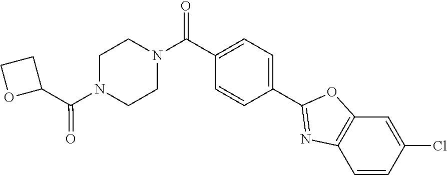 [SMILES: O=C(c1ccc(-c2nc3ccc(Cl)cc3o2)cc1)N1CCN(C(=O)C2CCO2)CC1] |
| 450 | 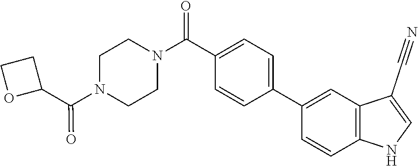 [SMILES: N#Cc1c[nH]c2ccc(-c3ccc(C(=O)N4CCN(C(=O)C5CCO5)CC4)cc3)cc12] |
| 451 | 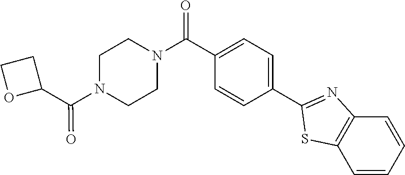 [SMILES: O=C(c1ccc(-c2nc3ccccc3s2)cc1)N1CCN(C(=O)C2CCO2)CC1] |
| 452 | 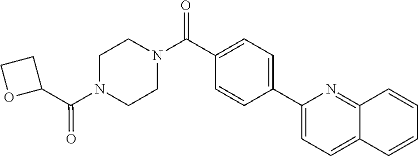 [SMILES: O=C(c1ccc(-c2ccc3ccccc3n2)cc1)N1CCN(C(=O)C2CCO2)CC1] |
| 453 | 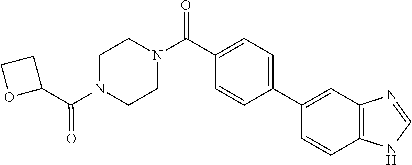 [SMILES: O=C(c1ccc(-c2ccc3[nH]cnc3c2)cc1)N1CCN(C(=O)C2CCO2)CC1] |
| 454 | 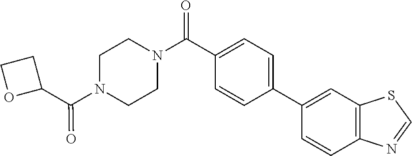 [SMILES: O=C(c1ccc(-c2ccc3ncsc3c2)cc1)N1CCN(C(=O)C2CCO2)CC1] |
| 455 | 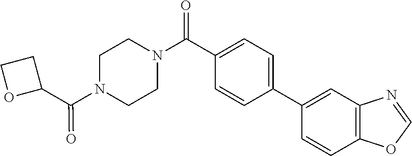 [SMILES: O=C(c1ccc(-c2ccc3ocnc3c2)cc1)N1CCN(C(=O)C2CCO2)CC1] |
| 456 | 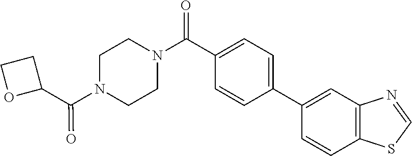 [SMILES: O=C(c1ccc(-c2ccc3scnc3c2)cc1)N1CCN(C(=O)C2CCO2)CC1] |
| 457 | 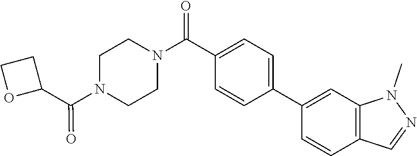 [SMILES: Cn1ncc2ccc(-c3ccc(C(=O)N4CCN(C(=O)C5CCO5)CC4)cc3)cc21] |
| 458 | 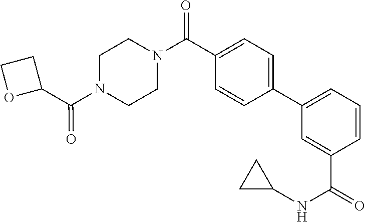 [SMILES: O=C(NC1CC1)c1cccc(-c2ccc(C(=O)N3CCN(C(=O)C4CCO4)CC3)cc2)c1] |
| 459 | 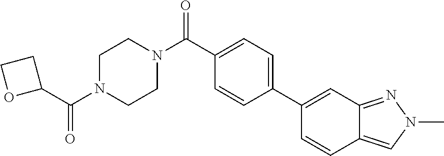 [SMILES: Cn1cc2ccc(-c3ccc(C(=O)N4CCN(C(=O)C5CCO5)CC4)cc3)cc2n1] |
| 460 | 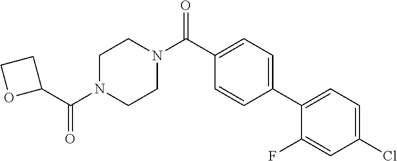 [SMILES: O=C(c1ccc(-c2ccc(Cl)cc2F)cc1)N1CCN(C(=O)C2CCO2)CC1] |
| 461 | 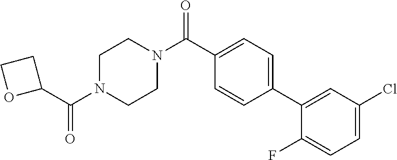 [SMILES: O=C(c1ccc(-c2cc(Cl)ccc2F)cc1)N1CCN(C(=O)C2CCO2)CC1] |
| 462 | 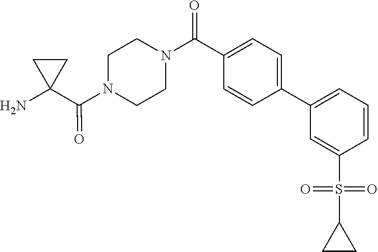 [SMILES: NC1(C(=O)N2CCN(C(=O)c3ccc(-c4cccc(S(=O)(=O)C5CC5)c4)cc3)CC2)CC1] |
| 463 | 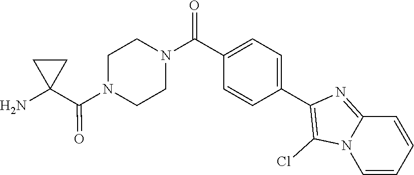 [SMILES: NC1(C(=O)N2CCN(C(=O)c3ccc(-c4nc5ccccn5c4Cl)cc3)CC2)CC1] |
| 464 | 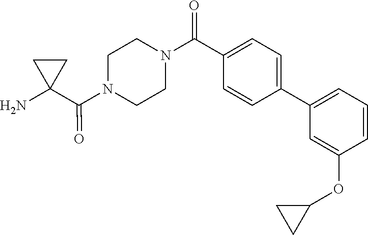 [SMILES: NC1(C(=O)N2CCN(C(=O)c3ccc(-c4cccc(OC5CC5)c4)cc3)CC2)CC1] |
| 465 | 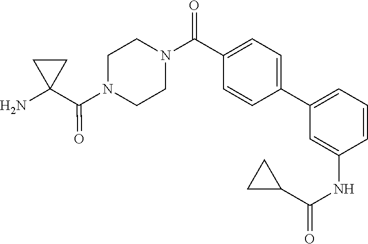 [SMILES: NC1(C(=O)N2CCN(C(=O)c3ccc(-c4cccc(NC(=O)C5CC5)c4)cc3)CC2)CC1] |
| 466 | 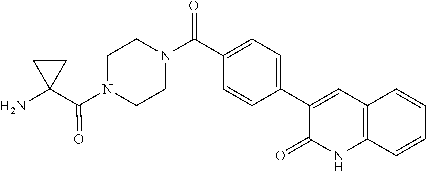 [SMILES: NC1(C(=O)N2CCN(C(=O)c3ccc(-c4cc5ccccc5[nH]c4=O)cc3)CC2)CC1] |
| 467 | 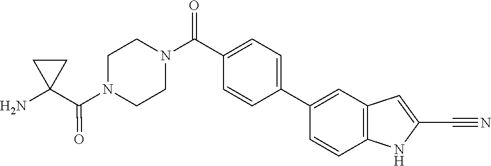 [SMILES: N#Cc1cc2cc(-c3ccc(C(=O)N4CCN(C(=O)C5(N)CC5)CC4)cc3)ccc2[nH]1] |
| 468 | 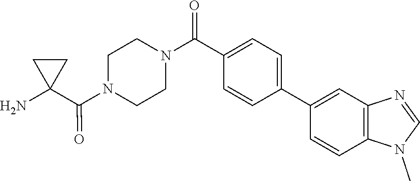 [SMILES: Cn1cnc2cc(-c3ccc(C(=O)N4CCN(C(=O)C5(N)CC5)CC4)cc3)ccc21] |
| 469 | 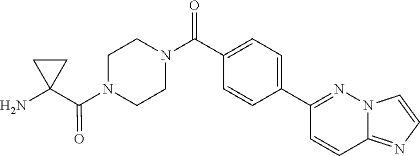 [SMILES: NC1(C(=O)N2CCN(C(=O)c3ccc(-c4ccc5nccn5n4)cc3)CC2)CC1] |
| 470 | 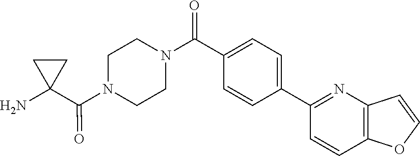 [SMILES: NC1(C(=O)N2CCN(C(=O)c3ccc(-c4ccc5occc5n4)cc3)CC2)CC1] |
| 471 | 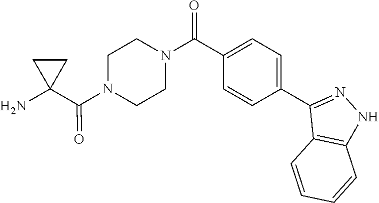 [SMILES: NC1(C(=O)N2CCN(C(=O)c3ccc(-c4n[nH]c5ccccc45)cc3)CC2)CC1] |
| 472 | 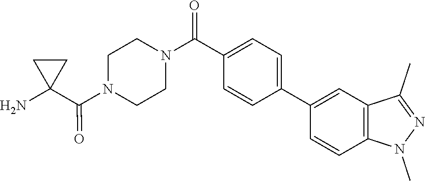 [SMILES: Cc1nn(C)c2ccc(-c3ccc(C(=O)N4CCN(C(=O)C5(N)CC5)CC4)cc3)cc12] |
| 473 | 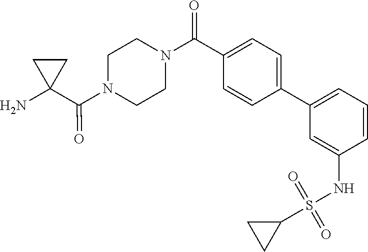 [SMILES: NC1(C(=O)N2CCN(C(=O)c3ccc(-c4cccc(NS(=O)(=O)C5CC5)c4)cc3)CC2)CC1] |
| 474 | 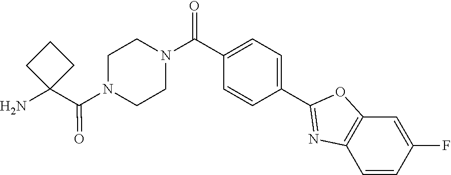 [SMILES: NC1(C(=O)N2CCN(C(=O)c3ccc(-c4nc5ccc(F)cc5o4)cc3)CC2)CCC1] |
| 475 | 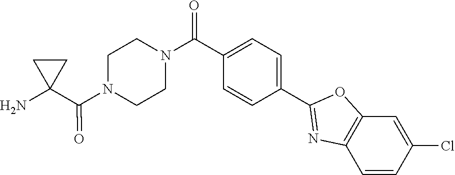 [SMILES: NC1(C(=O)N2CCN(C(=O)c3ccc(-c4nc5ccc(Cl)cc5o4)cc3)CC2)CC1] |
| 476 | 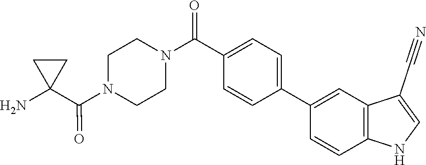 [SMILES: N#Cc1c[nH]c2ccc(-c3ccc(C(=O)N4CCN(C(=O)C5(N)CC5)CC4)cc3)cc12] |
| 477 | 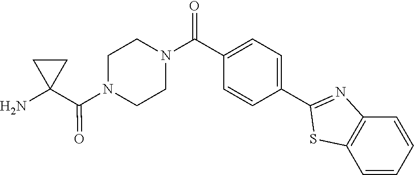 [SMILES: NC1(C(=O)N2CCN(C(=O)c3ccc(-c4nc5ccccc5s4)cc3)CC2)CC1] |
| 478 | 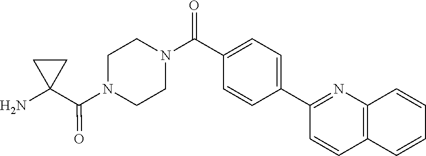 [SMILES: NC1(C(=O)N2CCN(C(=O)c3ccc(-c4ccc5ccccc5n4)cc3)CC2)CC1] |
| 479 | 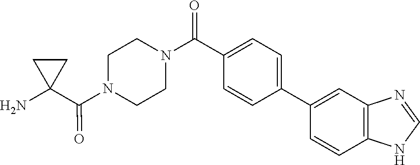 [SMILES: NC1(C(=O)N2CCN(C(=O)c3ccc(-c4ccc5[nH]cnc5c4)cc3)CC2)CC1] |
| 480 | 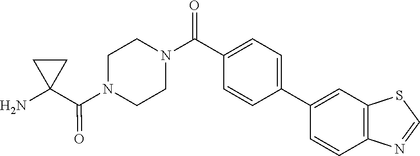 [SMILES: NC1(C(=O)N2CCN(C(=O)c3ccc(-c4ccc5ncsc5c4)cc3)CC2)CC1] |
| 481 | 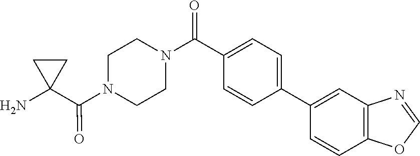 [SMILES: NC1(C(=O)N2CCN(C(=O)c3ccc(-c4ccc5ocnc5c4)cc3)CC2)CC1] |
| 482 | 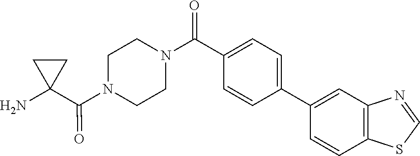 [SMILES: NC1(C(=O)N2CCN(C(=O)c3ccc(-c4ccc5scnc5c4)cc3)CC2)CC1] |
| 483 | 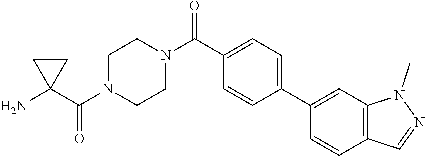 [SMILES: Cn1ncc2ccc(-c3ccc(C(=O)N4CCN(C(=O)C5(N)CC5)CC4)cc3)cc21] |
| 484 | 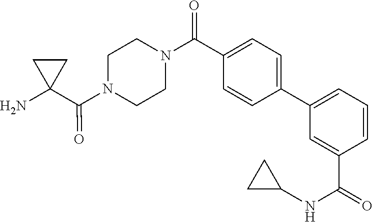 [SMILES: NC1(C(=O)N2CCN(C(=O)c3ccc(-c4cccc(C(=O)NC5CC5)c4)cc3)CC2)CC1] |
| 485 | 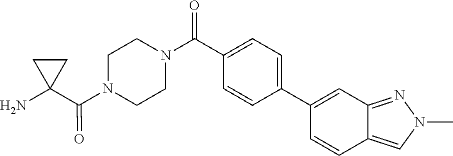 [SMILES: Cn1cc2ccc(-c3ccc(C(=O)N4CCN(C(=O)C5(N)CC5)CC4)cc3)cc2n1] |
| 486 | 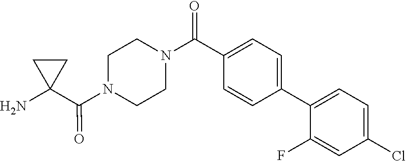 [SMILES: NC1(C(=O)N2CCN(C(=O)c3ccc(-c4ccc(Cl)cc4F)cc3)CC2)CC1] |
| 487 | 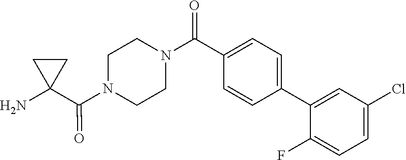 [SMILES: NC1(C(=O)N2CCN(C(=O)c3ccc(-c4cc(Cl)ccc4F)cc3)CC2)CC1] |
| 488 | 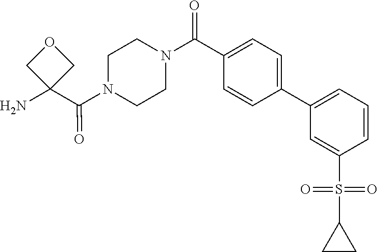 [SMILES: NC1(C(=O)N2CCN(C(=O)c3ccc(-c4cccc(S(=O)(=O)C5CC5)c4)cc3)CC2)COC1] |
| 489 | 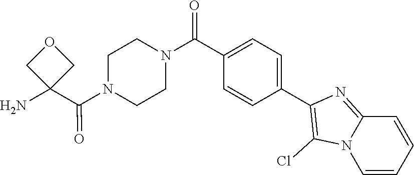 [SMILES: NC1(C(=O)N2CCN(C(=O)c3ccc(-c4nc5ccccn5c4Cl)cc3)CC2)COC1] |
| 490 | 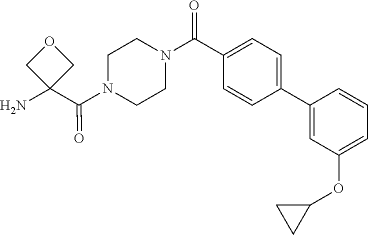 [SMILES: NC1(C(=O)N2CCN(C(=O)c3ccc(-c4cccc(OC5CC5)c4)cc3)CC2)COC1] |
| 491 | 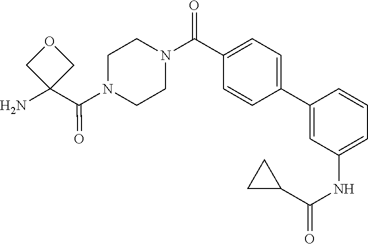 [SMILES: NC1(C(=O)N2CCN(C(=O)c3ccc(-c4cccc(NC(=O)C5CC5)c4)cc3)CC2)COC1] |
| 492 | 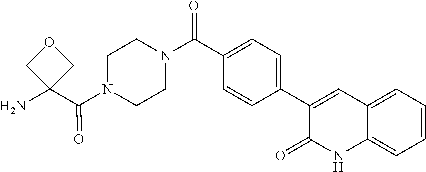 [SMILES: NC1(C(=O)N2CCN(C(=O)c3ccc(-c4cc5ccccc5[nH]c4=O)cc3)CC2)COC1] |
| 493 | 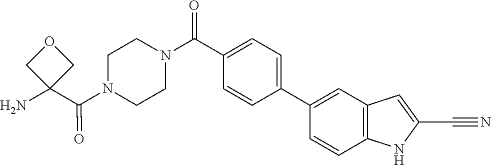 [SMILES: N#Cc1cc2cc(-c3ccc(C(=O)N4CCN(C(=O)C5(N)COC5)CC4)cc3)ccc2[nH]1] |
| 494 | 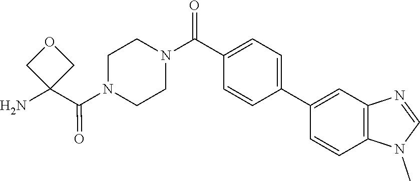 [SMILES: Cn1cnc2cc(-c3ccc(C(=O)N4CCN(C(=O)C5(N)COC5)CC4)cc3)ccc21] |
| 495 | 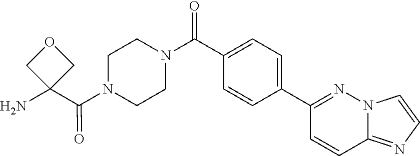 [SMILES: NC1(C(=O)N2CCN(C(=O)c3ccc(-c4ccc5nccn5n4)cc3)CC2)COC1] |
| 496 | 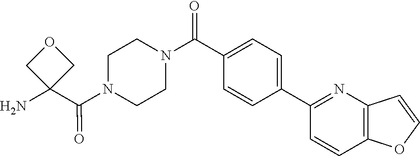 [SMILES: NC1(C(=O)N2CCN(C(=O)c3ccc(-c4ccc5occc5n4)cc3)CC2)COC1] |
| 497 | 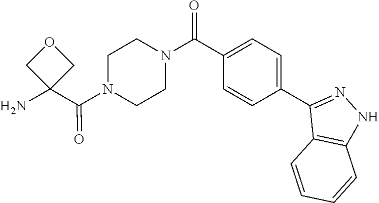 [SMILES: NC1(C(=O)N2CCN(C(=O)c3ccc(-c4n[nH]c5ccccc45)cc3)CC2)COC1] |
| 498 | 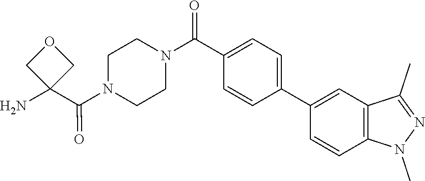 [SMILES: Cc1nn(C)c2ccc(-c3ccc(C(=O)N4CCN(C(=O)C5(N)COC5)CC4)cc3)cc12] |
| 499 | 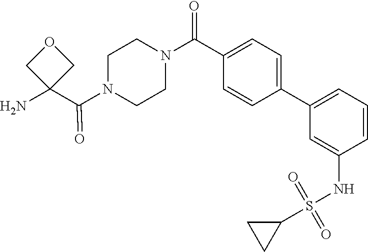 [SMILES: NC1(C(=O)N2CCN(C(=O)c3ccc(-c4cccc(NS(=O)(=O)C5CC5)c4)cc3)CC2)COC1] |
| 500 | 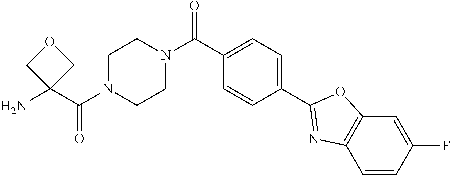 [SMILES: NC1(C(=O)N2CCN(C(=O)c3ccc(-c4nc5ccc(F)cc5o4)cc3)CC2)COC1] |
| 501 | 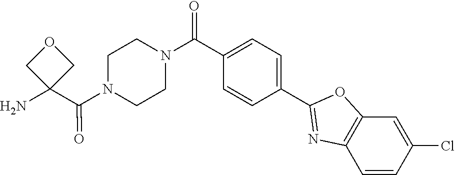 [SMILES: NC1(C(=O)N2CCN(C(=O)c3ccc(-c4nc5ccc(Cl)cc5o4)cc3)CC2)COC1] |
| 502 | 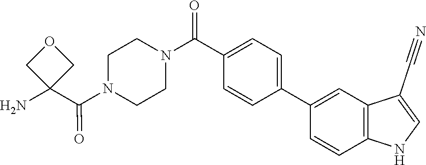 [SMILES: N#Cc1c[nH]c2ccc(-c3ccc(C(=O)N4CCN(C(=O)C5(N)COC5)CC4)cc3)cc12] |
| 503 | 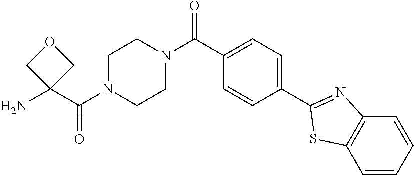 [SMILES: NC1(C(=O)N2CCN(C(=O)c3ccc(-c4nc5ccccc5s4)cc3)CC2)COC1] |
| 504 | 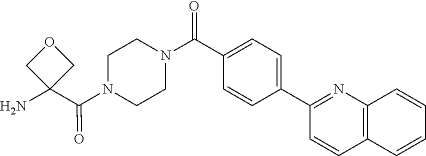 [SMILES: NC1(C(=O)N2CCN(C(=O)c3ccc(-c4ccc5ccccc5n4)cc3)CC2)COC1] |
| 505 | 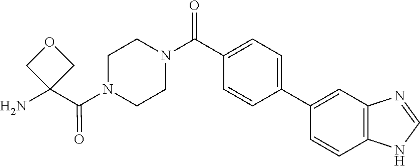 [SMILES: NC1(C(=O)N2CCN(C(=O)c3ccc(-c4ccc5[nH]cnc5c4)cc3)CC2)COC1] |
| 506 | 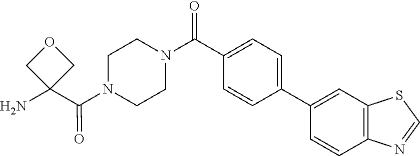 [SMILES: NC1(C(=O)N2CCN(C(=O)c3ccc(-c4ccc5ncsc5c4)cc3)CC2)COC1] |
| 507 | 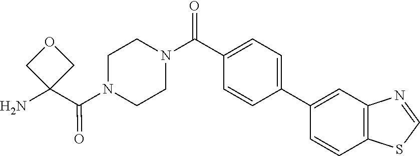 [SMILES: NC1(C(=O)N2CCN(C(=O)c3ccc(-c4ccc5scnc5c4)cc3)CC2)COC1] |
| 508 | 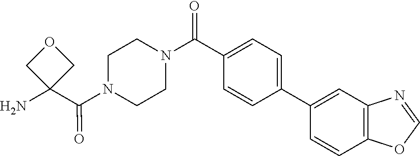 [SMILES: NC1(C(=O)N2CCN(C(=O)c3ccc(-c4ccc5ocnc5c4)cc3)CC2)COC1] |
| 509 | 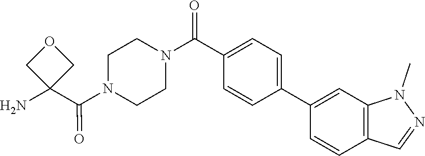 [SMILES: Cn1ncc2ccc(-c3ccc(C(=O)N4CCN(C(=O)C5(N)COC5)CC4)cc3)cc21] |
| 510 | 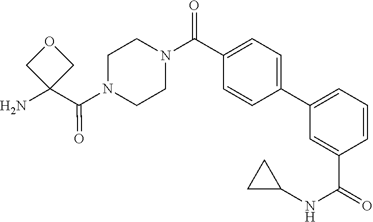 [SMILES: NC1(C(=O)N2CCN(C(=O)c3ccc(-c4cccc(C(=O)NC5CC5)c4)cc3)CC2)COC1] |
| 511 | 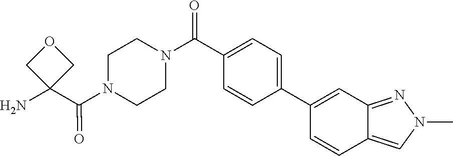 [SMILES: Cn1cc2ccc(-c3ccc(C(=O)N4CCN(C(=O)C5(N)COC5)CC4)cc3)cc2n1] |
| 512 | 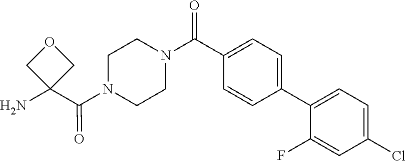 [SMILES: NC1(C(=O)N2CCN(C(=O)c3ccc(-c4ccc(Cl)cc4F)cc3)CC2)COC1] |
| 513 | 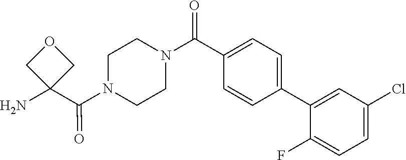 [SMILES: NC1(C(=O)N2CCN(C(=O)c3ccc(-c4cc(Cl)ccc4F)cc3)CC2)COC1] |
| 514 | 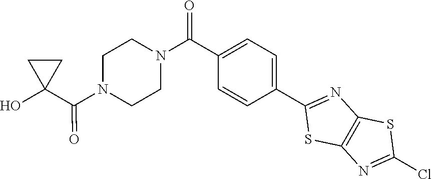 [SMILES: O=C(c1ccc(-c2nc3sc(Cl)nc3s2)cc1)N1CCN(C(=O)C2(O)CC2)CC1] |
| 515 | 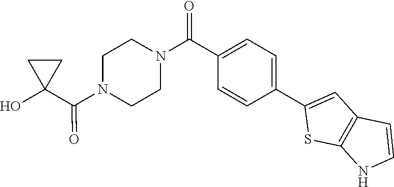 [SMILES: O=C(c1ccc(-c2cc3cc[nH]c3s2)cc1)N1CCN(C(=O)C2(O)CC2)CC1] |
| 516 | 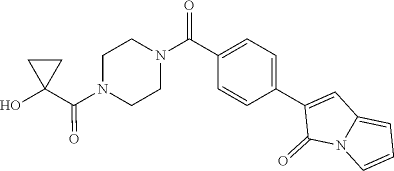 [SMILES: O=C(c1ccc(C2=Cc3cccn3C2=O)cc1)N1CCN(C(=O)C2(O)CC2)CC1] |
| 517 | 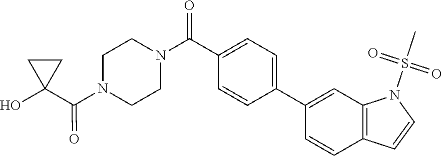 [SMILES: CS(=O)(=O)n1ccc2ccc(-c3ccc(C(=O)N4CCN(C(=O)C5(O)CC5)CC4)cc3)cc21] |
| 518 | 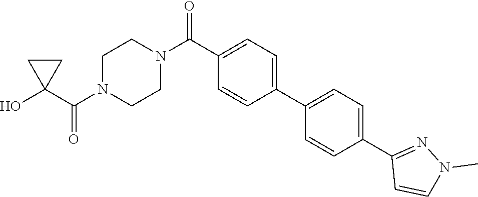 [SMILES: Cn1ccc(-c2ccc(-c3ccc(C(=O)N4CCN(C(=O)C5(O)CC5)CC4)cc3)cc2)n1] |
| 519 | 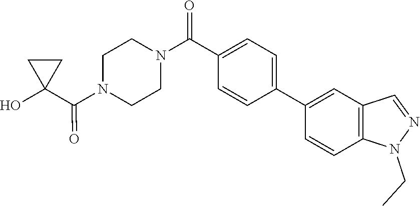 [SMILES: CCn1ncc2cc(-c3ccc(C(=O)N4CCN(C(=O)C5(O)CC5)CC4)cc3)ccc21] |
| 520 | 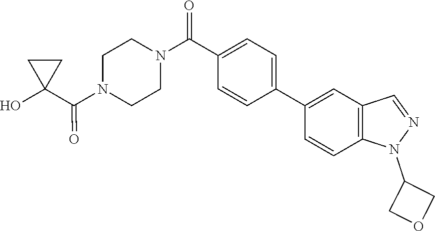 [SMILES: O=C(c1ccc(-c2ccc3c(cnn3C3COC3)c2)cc1)N1CCN(C(=O)C2(O)CC2)CC1] |
| 521 | 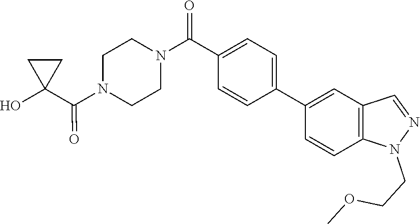 [SMILES: COCCn1ncc2cc(-c3ccc(C(=O)N4CCN(C(=O)C5(O)CC5)CC4)cc3)ccc21] |
| 522 | 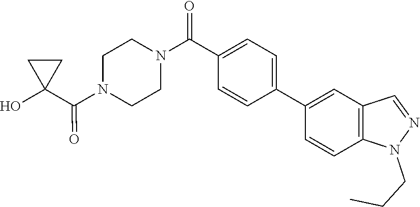 [SMILES: CCCn1ncc2cc(-c3ccc(C(=O)N4CCN(C(=O)C5(O)CC5)CC4)cc3)ccc21] |
| 523 | 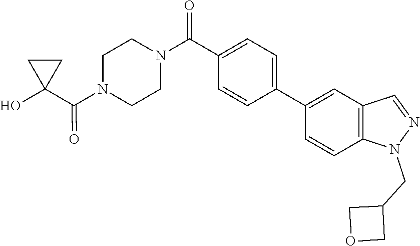 [SMILES: O=C(c1ccc(-c2ccc3c(cnn3CC3COC3)c2)cc1)N1CCN(C(=O)C2(O)CC2)CC1] |
| 524 | 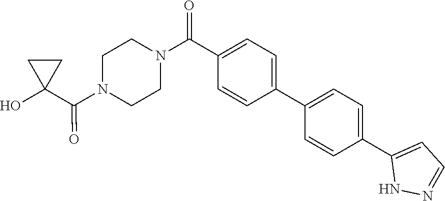 [SMILES: O=C(c1ccc(-c2ccc(-c3ccn[nH]3)cc2)cc1)N1CCN(C(=O)C2(O)CC2)CC1] |
| 525 | 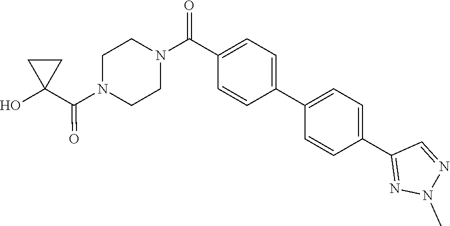 [SMILES: Cn1ncc(-c2ccc(-c3ccc(C(=O)N4CCN(C(=O)C5(O)CC5)CC4)cc3)cc2)n1] |
| 526 | 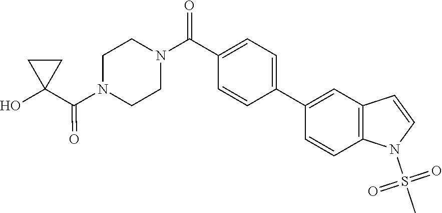 [SMILES: CS(=O)(=O)n1ccc2cc(-c3ccc(C(=O)N4CCN(C(=O)C5(O)CC5)CC4)cc3)ccc21] |
| 527 | 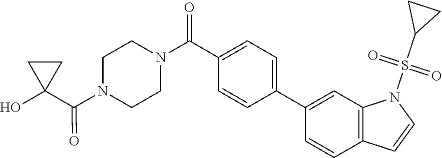 [SMILES: O=C(c1ccc(-c2ccc3ccn(S(=O)(=O)C4CC4)c3c2)cc1)N1CCN(C(=O)C2(O)CC2)CC1] |
| 528 | 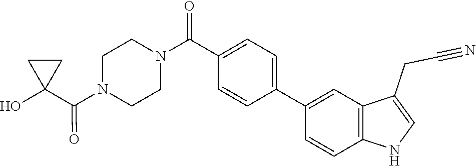 [SMILES: N#CCc1c[nH]c2ccc(-c3ccc(C(=O)N4CCN(C(=O)C5(O)CC5)CC4)cc3)cc12] |
| 529 | 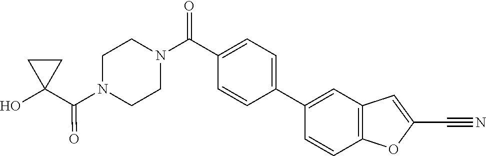 [SMILES: N#Cc1cc2cc(-c3ccc(C(=O)N4CCN(C(=O)C5(O)CC5)CC4)cc3)ccc2o1] |
| 530 | 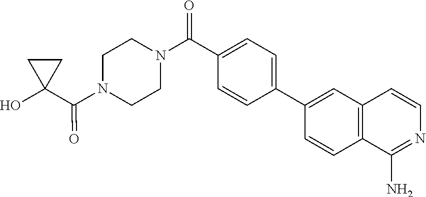 [SMILES: Nc1nccc2cc(-c3ccc(C(=O)N4CCN(C(=O)C5(O)CC5)CC4)cc3)ccc12] |
| 531 | 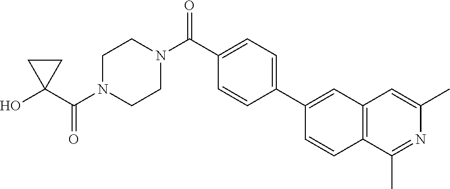 [SMILES: Cc1cc2cc(-c3ccc(C(=O)N4CCN(C(=O)C5(O)CC5)CC4)cc3)ccc2c(C)n1] |
| 532 | 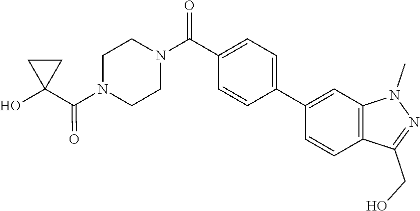 [SMILES: Cn1nc(CO)c2ccc(-c3ccc(C(=O)N4CCN(C(=O)C5(O)CC5)CC4)cc3)cc21] |
| 533 | 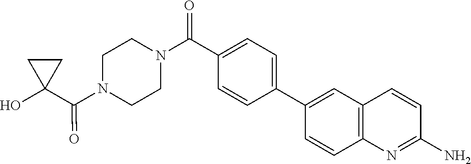 [SMILES: Nc1ccc2cc(-c3ccc(C(=O)N4CCN(C(=O)C5(O)CC5)CC4)cc3)ccc2n1] |
| 534 | 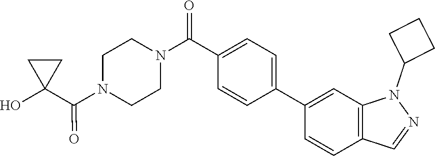 [SMILES: O=C(c1ccc(-c2ccc3cnn(C4CCC4)c3c2)cc1)N1CCN(C(=O)C2(O)CC2)CC1] |
| 535 | 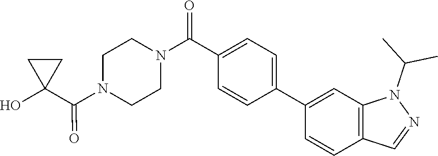 [SMILES: CC(C)n1ncc2ccc(-c3ccc(C(=O)N4CCN(C(=O)C5(O)CC5)CC4)cc3)cc21] |
| 536 | 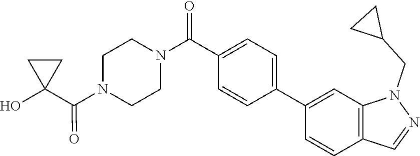 [SMILES: O=C(c1ccc(-c2ccc3cnn(CC4CC4)c3c2)cc1)N1CCN(C(=O)C2(O)CC2)CC1] |
| 537 | 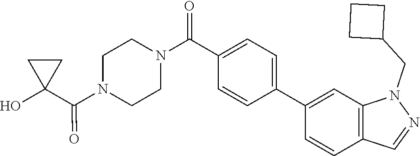 [SMILES: O=C(c1ccc(-c2ccc3cnn(CC4CCC4)c3c2)cc1)N1CCN(C(=O)C2(O)CC2)CC1] |
| 538 | 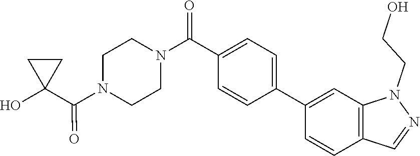 [SMILES: O=C(c1ccc(-c2ccc3cnn(CCO)c3c2)cc1)N1CCN(C(=O)C2(O)CC2)CC1] |
| 539 | 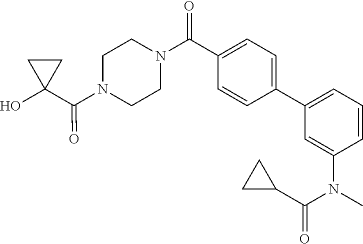 [SMILES: CN(C(=O)C1CC1)c1cccc(-c2ccc(C(=O)N3CCN(C(=O)C4(O)CC4)CC3)cc2)c1] |
| 540 | 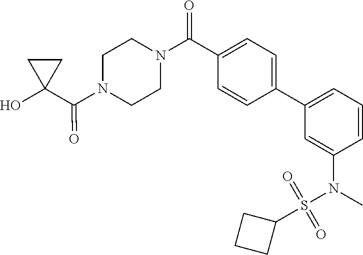 [SMILES: CN(c1cccc(-c2ccc(C(=O)N3CCN(C(=O)C4(O)CC4)CC3)cc2)c1)S(=O)(=O)C1CCC1] |
| 541 | 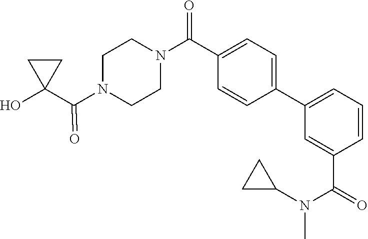 [SMILES: CN(C(=O)c1cccc(-c2ccc(C(=O)N3CCN(C(=O)C4(O)CC4)CC3)cc2)c1)C1CC1] |
| 542 | 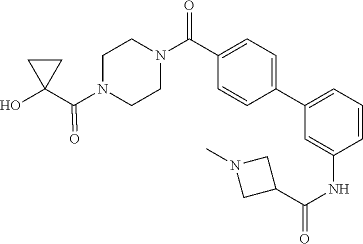 [SMILES: CN1CC(C(=O)Nc2cccc(-c3ccc(C(=O)N4CCN(C(=O)C5(O)CC5)CC4)cc3)c2)C1] |
| 543 | 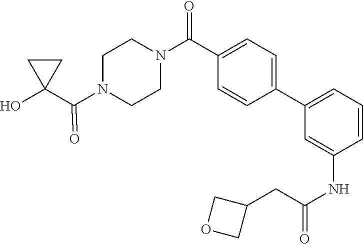 [SMILES: O=C(CC1COC1)Nc1cccc(-c2ccc(C(=O)N3CCN(C(=O)C4(O)CC4)CC3)cc2)c1] |
| 544 | 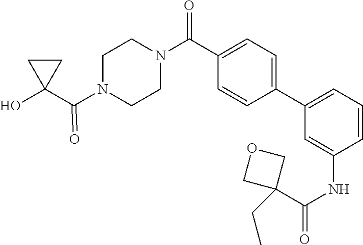 [SMILES: CCC1(C(=O)Nc2cccc(-c3ccc(C(=O)N4CCN(C(=O)C5(O)CC5)CC4)cc3)c2)COC1] |
| 545 | 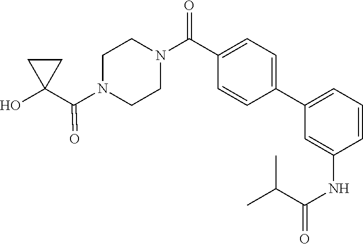 [SMILES: CC(C)C(=O)Nc1cccc(-c2ccc(C(=O)N3CCN(C(=O)C4(O)CC4)CC3)cc2)c1] |
| 546 | 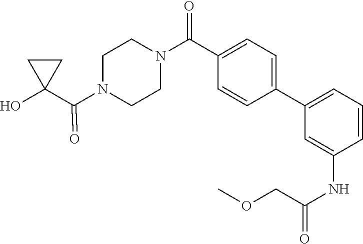 [SMILES: COCC(=O)Nc1cccc(-c2ccc(C(=O)N3CCN(C(=O)C4(O)CC4)CC3)cc2)c1] |
| 547 | 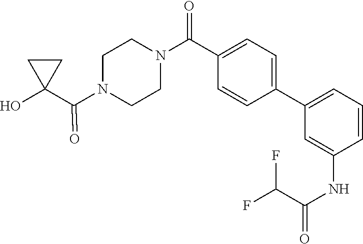 [SMILES: O=C(Nc1cccc(-c2ccc(C(=O)N3CCN(C(=O)C4(O)CC4)CC3)cc2)c1)C(F)F] |
| 548 | 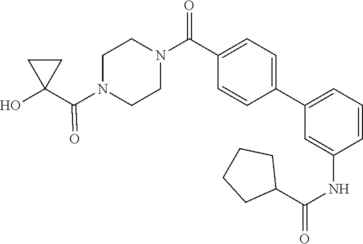 [SMILES: O=C(Nc1cccc(-c2ccc(C(=O)N3CCN(C(=O)C4(O)CC4)CC3)cc2)c1)C1CCCC1] |
| 549 | 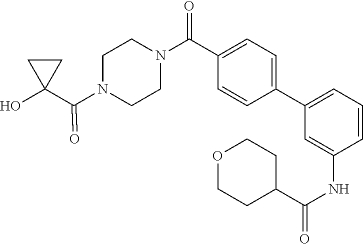 [SMILES: O=C(Nc1cccc(-c2ccc(C(=O)N3CCN(C(=O)C4(O)CC4)CC3)cc2)c1)C1CCOCC1] |
| 550 | 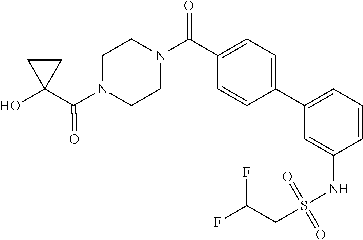 [SMILES: O=C(c1ccc(-c2cccc(NS(=O)(=O)CC(F)F)c2)cc1)N1CCN(C(=O)C2(O)CC2)CC1] |
| 551 | 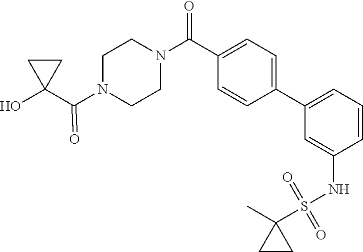 [SMILES: CC1(S(=O)(=O)Nc2cccc(-c3ccc(C(=O)N4CCN(C(=O)C5(O)CC5)CC4)cc3)c2)CC1] |
| 552 | 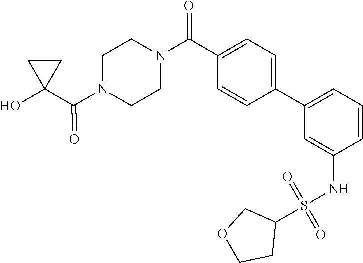 [SMILES: O=C(c1ccc(-c2cccc(NS(=O)(=O)C3CCOC3)c2)cc1)N1CCN(C(=O)C2(O)CC2)CC1] |
| 553 | 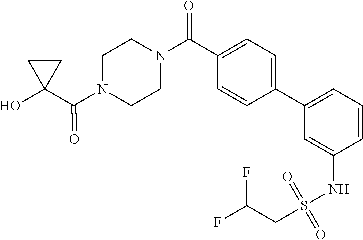 [SMILES: O=C(c1ccc(-c2cccc(NS(=O)(=O)CC(F)F)c2)cc1)N1CCN(C(=O)C2(O)CC2)CC1] |
| 554 | 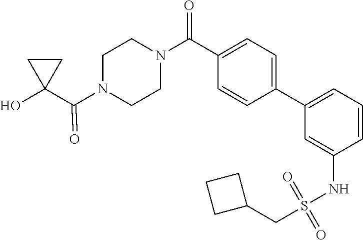 [SMILES: O=C(c1ccc(-c2cccc(NS(=O)(=O)CC3CCC3)c2)cc1)N1CCN(C(=O)C2(O)CC2)CC1] |
| 555 | 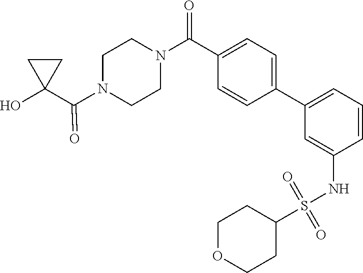 [SMILES: O=C(c1ccc(-c2cccc(NS(=O)(=O)C3CCOCC3)c2)cc1)N1CCN(C(=O)C2(O)CC2)CC1] |
| 556 | 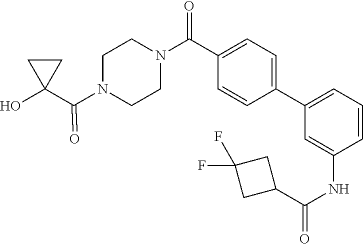 [SMILES: O=C(Nc1cccc(-c2ccc(C(=O)N3CCN(C(=O)C4(O)CC4)CC3)cc2)c1)C1CC(F)(F)C1] |
| 557 | 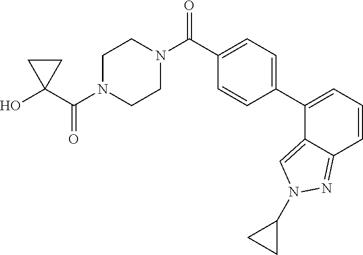 [SMILES: O=C(c1ccc(-c2cccc3nn(C4CC4)cc23)cc1)N1CCN(C(=O)C2(O)CC2)CC1] |
| 558 | 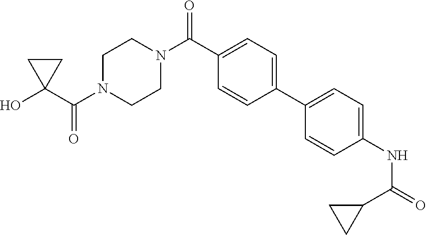 [SMILES: O=C(Nc1ccc(-c2ccc(C(=O)N3CCN(C(=O)C4(O)CC4)CC3)cc2)cc1)C1CC1] |
| 559 | 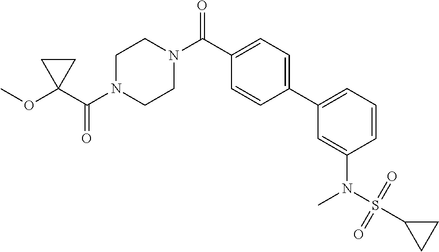 [SMILES: COC1(C(=O)N2CCN(C(=O)c3ccc(-c4cccc(N(C)S(=O)(=O)C5CC5)c4)cc3)CC2)CC1] |
| 560 | 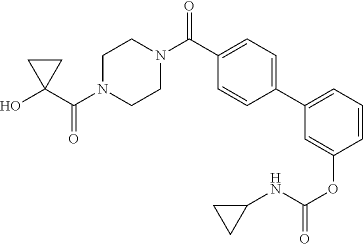 [SMILES: O=C(NC1CC1)Oc1cccc(-c2ccc(C(=O)N3CCN(C(=O)C4(O)CC4)CC3)cc2)c1] |
| 561 | 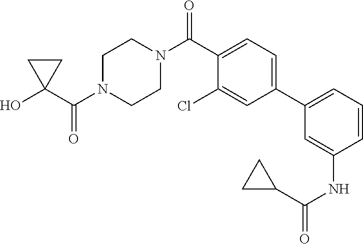 [SMILES: O=C(Nc1cccc(-c2ccc(C(=O)N3CCN(C(=O)C4(O)CC4)CC3)c(Cl)c2)c1)C1CC1] |
| 562 | 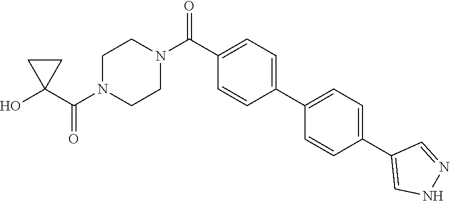 [SMILES: O=C(c1ccc(-c2ccc(-c3cn[nH]c3)cc2)cc1)N1CCN(C(=O)C2(O)CC2)CC1] |
| 563 | 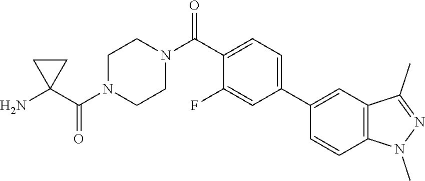 [SMILES: Cc1nn(C)c2ccc(-c3ccc(C(=O)N4CCN(C(=O)C5(N)CC5)CC4)c(F)c3)cc12] |
| 564 | 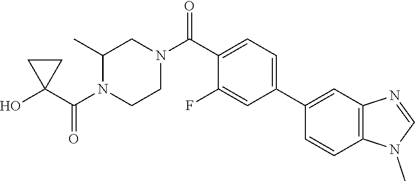 [SMILES: CC1CN(C(=O)c2ccc(-c3ccc4c(c3)ncn4C)cc2F)CCN1C(=O)C1(O)CC1] |
| 565 | 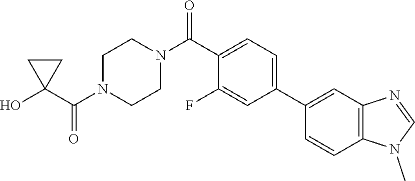 [SMILES: Cn1cnc2cc(-c3ccc(C(=O)N4CCN(C(=O)C5(O)CC5)CC4)c(F)c3)ccc21] |
| 566 | 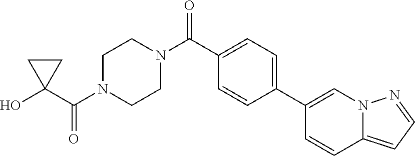 [SMILES: O=C(c1ccc(-c2ccc3ccnn3c2)cc1)N1CCN(C(=O)C2(O)CC2)CC1] |
| 567 | 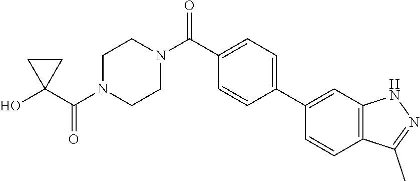 [SMILES: Cc1n[nH]c2cc(-c3ccc(C(=O)N4CCN(C(=O)C5(O)CC5)CC4)cc3)ccc12] |
| 568 | 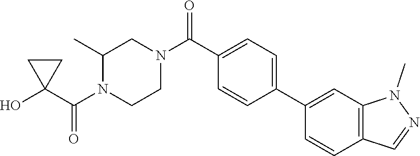 [SMILES: CC1CN(C(=O)c2ccc(-c3ccc4cnn(C)c4c3)cc2)CCN1C(=O)C1(O)CC1] |
| 569 | 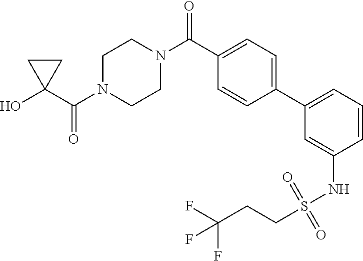 [SMILES: O=C(c1ccc(-c2cccc(NS(=O)(=O)CCC(F)(F)F)c2)cc1)N1CCN(C(=O)C2(O)CC2)CC1] |
| 570 | 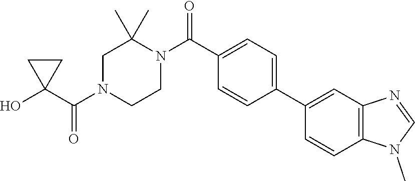 [SMILES: Cn1cnc2cc(-c3ccc(C(=O)N4CCN(C(=O)C5(O)CC5)CC4(C)C)cc3)ccc21] |
| 571 | 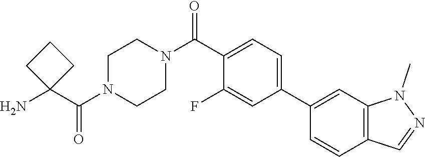 [SMILES: Cn1ncc2ccc(-c3ccc(C(=O)N4CCN(C(=O)C5(N)CCC5)CC4)c(F)c3)cc21] |
| 572 | 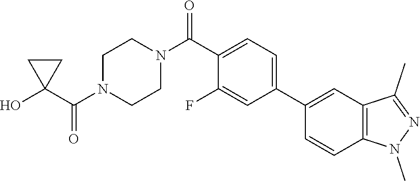 [SMILES: Cc1nn(C)c2ccc(-c3ccc(C(=O)N4CCN(C(=O)C5(O)CC5)CC4)c(F)c3)cc12] |
| 573 | 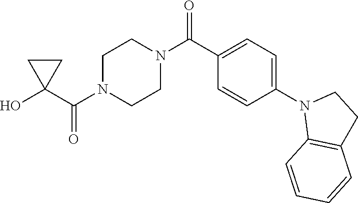 [SMILES: O=C(c1ccc(N2CCc3ccccc32)cc1)N1CCN(C(=O)C2(O)CC2)CC1] |
| 574 | 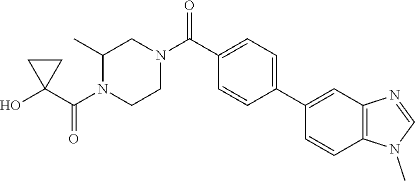 [SMILES: CC1CN(C(=O)c2ccc(-c3ccc4c(c3)ncn4C)cc2)CCN1C(=O)C1(O)CC1] |
| 575 | 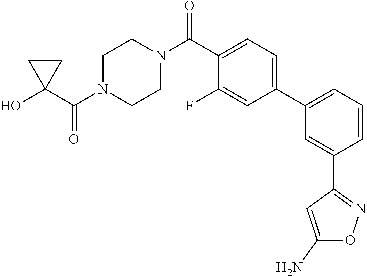 [SMILES: Nc1cc(-c2cccc(-c3ccc(C(=O)N4CCN(C(=O)C5(O)CC5)CC4)c(F)c3)c2)no1] |
| 576 | 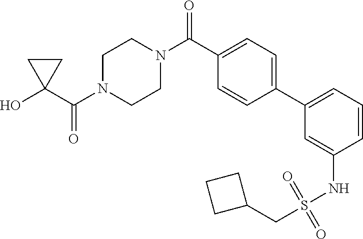 [SMILES: O=C(c1ccc(-c2cccc(NS(=O)(=O)CC3CCC3)c2)cc1)N1CCN(C(=O)C2(O)CC2)CC1] |
| 577 | 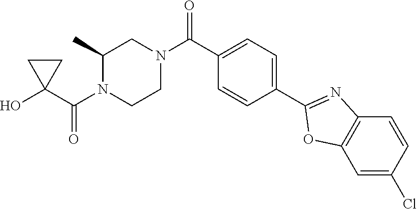 [SMILES: C[C@H]1CN(C(=O)c2ccc(-c3nc4ccc(Cl)cc4o3)cc2)CCN1C(=O)C1(O)CC1] |
| 578 | 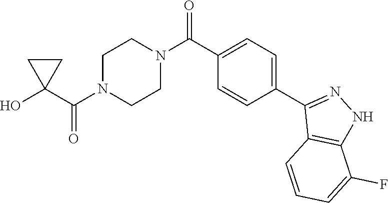 [SMILES: O=C(c1ccc(-c2n[nH]c3c(F)cccc23)cc1)N1CCN(C(=O)C2(O)CC2)CC1] |
| 579 | 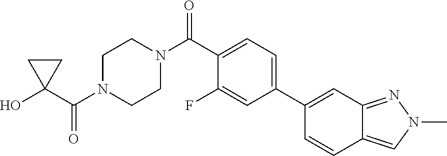 [SMILES: Cn1cc2ccc(-c3ccc(C(=O)N4CCN(C(=O)C5(O)CC5)CC4)c(F)c3)cc2n1] |
| 580 | 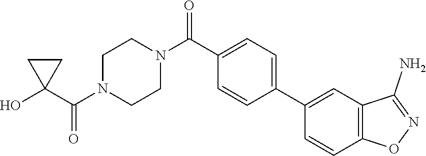 [SMILES: Nc1noc2ccc(-c3ccc(C(=O)N4CCN(C(=O)C5(O)CC5)CC4)cc3)cc12] |
| 581 | 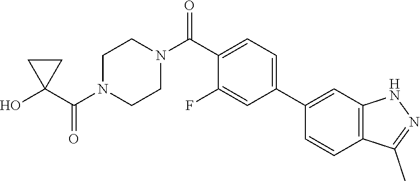 [SMILES: Cc1n[nH]c2cc(-c3ccc(C(=O)N4CCN(C(=O)C5(O)CC5)CC4)c(F)c3)ccc12] |
| 582 | 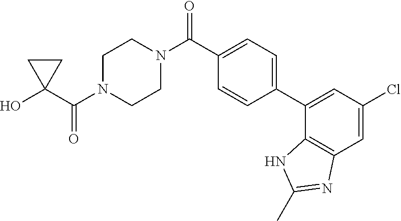 [SMILES: Cc1nc2cc(Cl)cc(-c3ccc(C(=O)N4CCN(C(=O)C5(O)CC5)CC4)cc3)c2[nH]1] |
| 583 | 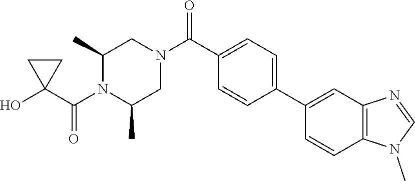 [SMILES: C[C@@H]1CN(C(=O)c2ccc(-c3ccc4c(c3)ncn4C)cc2)C[C@H](C)N1C(=O)C1(O)CC1] |
| 584 | 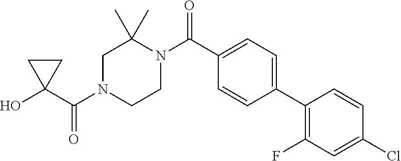 [SMILES: CC1(C)CN(C(=O)C2(O)CC2)CCN1C(=O)c1ccc(-c2ccc(Cl)cc2F)cc1] |
| 585 | 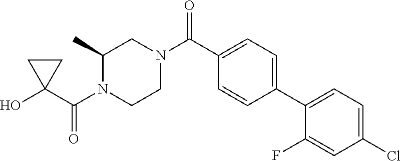 [SMILES: C[C@H]1CN(C(=O)c2ccc(-c3ccc(Cl)cc3F)cc2)CCN1C(=O)C1(O)CC1] |
| 586 | 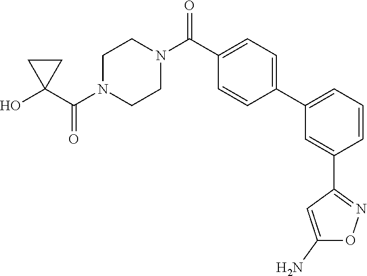 [SMILES: Nc1cc(-c2cccc(-c3ccc(C(=O)N4CCN(C(=O)C5(O)CC5)CC4)cc3)c2)no1] |
| 587 | 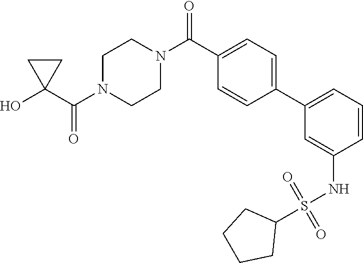 [SMILES: O=C(c1ccc(-c2cccc(NS(=O)(=O)C3CCCC3)c2)cc1)N1CCN(C(=O)C2(O)CC2)CC1] |
| 588 | 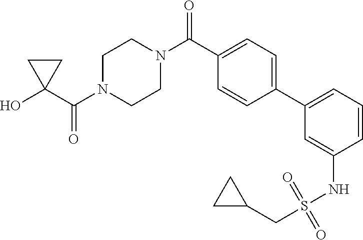 [SMILES: O=C(c1ccc(-c2cccc(NS(=O)(=O)CC3CC3)c2)cc1)N1CCN(C(=O)C2(O)CC2)CC1] |
| 589 | 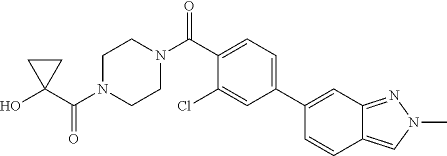 [SMILES: Cn1cc2ccc(-c3ccc(C(=O)N4CCN(C(=O)C5(O)CC5)CC4)c(Cl)c3)cc2n1] |
| 590 | 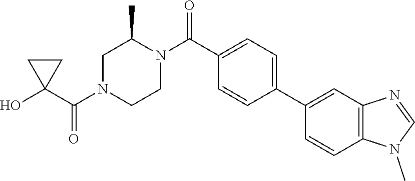 [SMILES: C[C@@H]1CN(C(=O)C2(O)CC2)CCN1C(=O)c1ccc(-c2ccc3c(c2)ncn3C)cc1] |
| 591 | 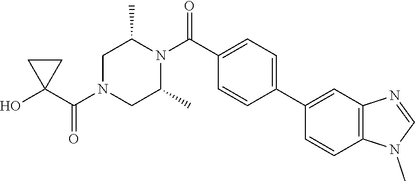 [SMILES: C[C@@H]1CN(C(=O)C2(O)CC2)C[C@H](C)N1C(=O)c1ccc(-c2ccc3c(c2)ncn3C)cc1] |
| 592 | 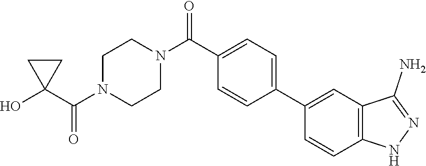 [SMILES: Nc1n[nH]c2ccc(-c3ccc(C(=O)N4CCN(C(=O)C5(O)CC5)CC4)cc3)cc12] |
| 593 | 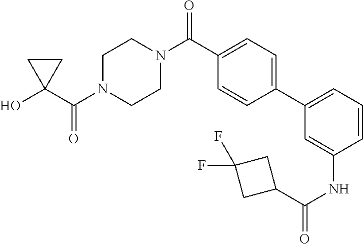 [SMILES: O=C(Nc1cccc(-c2ccc(C(=O)N3CCN(C(=O)C4(O)CC4)CC3)cc2)c1)C1CC(F)(F)C1] |
| 594 | 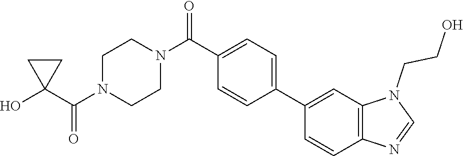 [SMILES: O=C(c1ccc(-c2ccc3ncn(CCO)c3c2)cc1)N1CCN(C(=O)C2(O)CC2)CC1] |
| 595 | 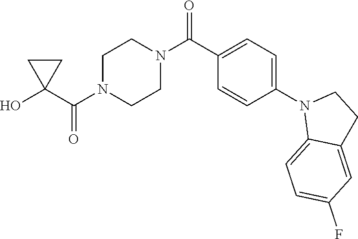 [SMILES: O=C(c1ccc(N2CCc3cc(F)ccc32)cc1)N1CCN(C(=O)C2(O)CC2)CC1] |
| 596 | 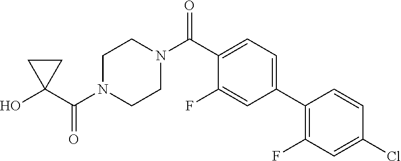 [SMILES: O=C(c1ccc(-c2ccc(Cl)cc2F)cc1F)N1CCN(C(=O)C2(O)CC2)CC1] |
| 597 | 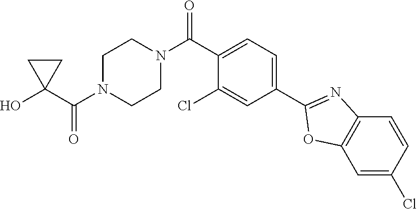 [SMILES: O=C(c1ccc(-c2nc3ccc(Cl)cc3o2)cc1Cl)N1CCN(C(=O)C2(O)CC2)CC1] |
| 598 | 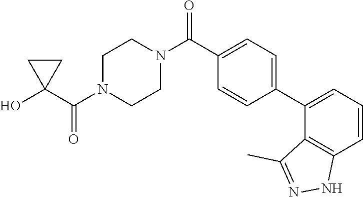 [SMILES: Cc1n[nH]c2cccc(-c3ccc(C(=O)N4CCN(C(=O)C5(O)CC5)CC4)cc3)c12] |
| 599 | 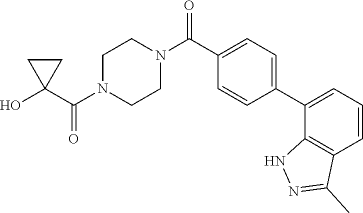 [SMILES: Cc1n[nH]c2c(-c3ccc(C(=O)N4CCN(C(=O)C5(O)CC5)CC4)cc3)cccc12] |
| 600 | 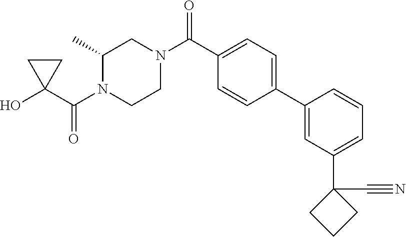 [SMILES: C[C@@H]1CN(C(=O)c2ccc(-c3cccc(C4(C#N)CCC4)c3)cc2)CCN1C(=O)C1(O)CC1] |
| 601 | 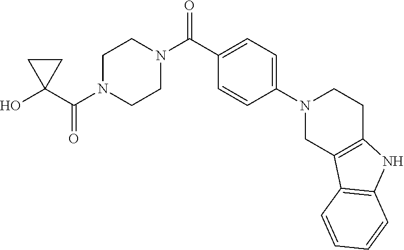 [SMILES: O=C(c1ccc(N2CCc3[nH]c4ccccc4c3C2)cc1)N1CCN(C(=O)C2(O)CC2)CC1] |
| 602 | 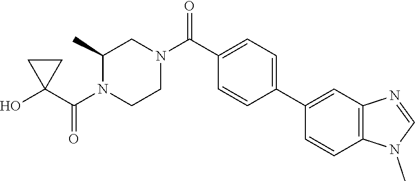 [SMILES: C[C@H]1CN(C(=O)c2ccc(-c3ccc4c(c3)ncn4C)cc2)CCN1C(=O)C1(O)CC1] |
| 603 | 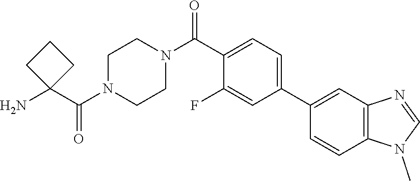 [SMILES: Cn1cnc2cc(-c3ccc(C(=O)N4CCN(C(=O)C5(N)CCC5)CC4)c(F)c3)ccc21] |
| 604 | 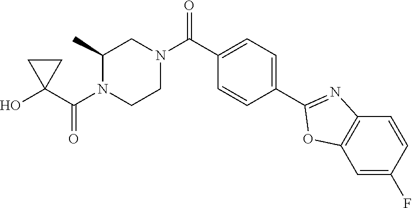 [SMILES: C[C@H]1CN(C(=O)c2ccc(-c3nc4ccc(F)cc4o3)cc2)CCN1C(=O)C1(O)CC1] |
| 605 | 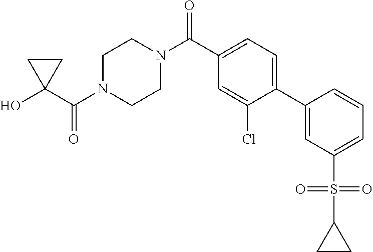 [SMILES: O=C(c1ccc(-c2cccc(S(=O)(=O)C3CC3)c2)c(Cl)c1)N1CCN(C(=O)C2(O)CC2)CC1] |
| 606 | 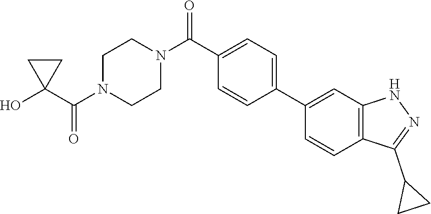 [SMILES: O=C(c1ccc(-c2ccc3c(C4CC4)n[nH]c3c2)cc1)N1CCN(C(=O)C2(O)CC2)CC1] |
| 607 | 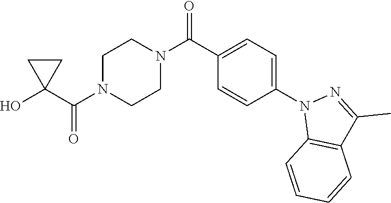 [SMILES: Cc1nn(-c2ccc(C(=O)N3CCN(C(=O)C4(O)CC4)CC3)cc2)c2ccccc12] |
| 608 | 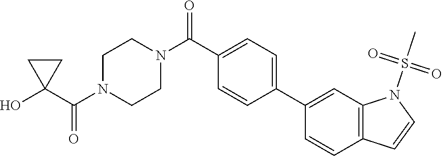 [SMILES: CS(=O)(=O)n1ccc2ccc(-c3ccc(C(=O)N4CCN(C(=O)C5(O)CC5)CC4)cc3)cc21] |
| 609 | 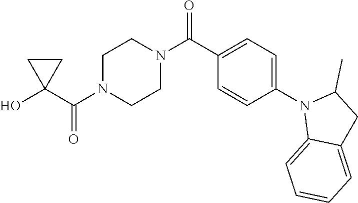 [SMILES: CC1Cc2ccccc2N1c1ccc(C(=O)N2CCN(C(=O)C3(O)CC3)CC2)cc1] |
| 610 | 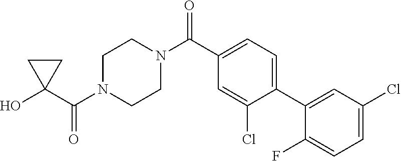 [SMILES: O=C(c1ccc(-c2cc(Cl)ccc2F)c(Cl)c1)N1CCN(C(=O)C2(O)CC2)CC1] |
| 611 | 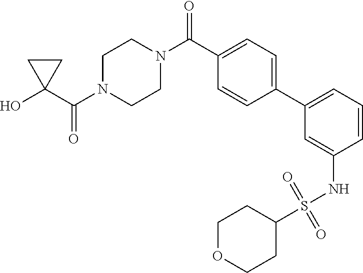 [SMILES: O=C(c1ccc(-c2cccc(NS(=O)(=O)C3CCOCC3)c2)cc1)N1CCN(C(=O)C2(O)CC2)CC1] |
| 612 | 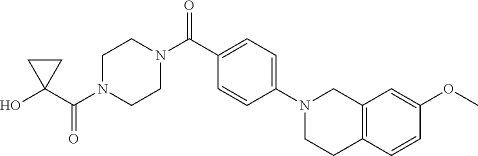 [SMILES: COc1ccc2c(c1)CN(c1ccc(C(=O)N3CCN(C(=O)C4(O)CC4)CC3)cc1)CC2] |
| 613 | 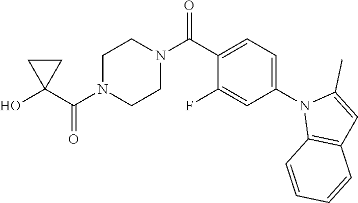 [SMILES: Cc1cc2ccccc2n1-c1ccc(C(=O)N2CCN(C(=O)C3(O)CC3)CC2)c(F)c1] |
| 614 | 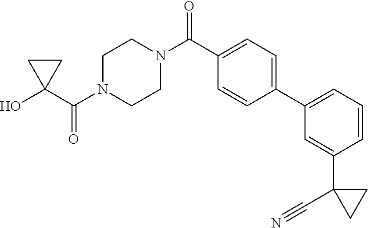 [SMILES: N#CC1(c2cccc(-c3ccc(C(=O)N4CCN(C(=O)C5(O)CC5)CC4)cc3)c2)CC1] |
| 615 | 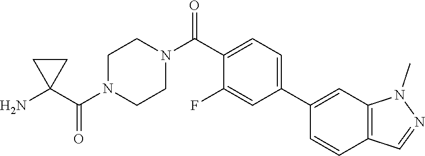 [SMILES: Cn1ncc2ccc(-c3ccc(C(=O)N4CCN(C(=O)C5(N)CC5)CC4)c(F)c3)cc21] |
| 616 | 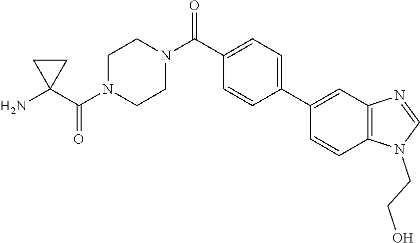 [SMILES: NC1(C(=O)N2CCN(C(=O)c3ccc(-c4ccc5c(c4)ncn5CCO)cc3)CC2)CC1] |
| 617 | 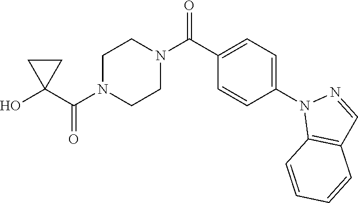 [SMILES: O=C(c1ccc(-n2ncc3ccccc32)cc1)N1CCN(C(=O)C2(O)CC2)CC1] |
| 618 | 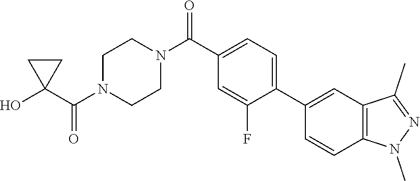 [SMILES: Cc1nn(C)c2ccc(-c3ccc(C(=O)N4CCN(C(=O)C5(O)CC5)CC4)cc3F)cc12] |
| 619 | 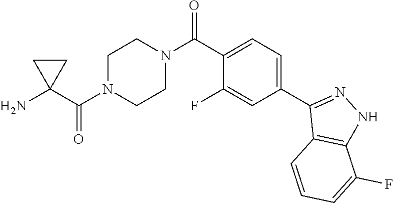 [SMILES: NC1(C(=O)N2CCN(C(=O)c3ccc(-c4n[nH]c5c(F)cccc45)cc3F)CC2)CC1] |
| 620 | 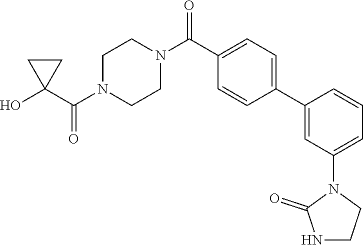 [SMILES: O=C(c1ccc(-c2cccc(N3CCNC3=O)c2)cc1)N1CCN(C(=O)C2(O)CC2)CC1] |
| 621 | 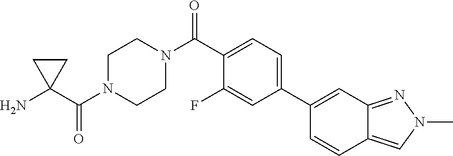 [SMILES: Cn1cc2ccc(-c3ccc(C(=O)N4CCN(C(=O)C5(N)CC5)CC4)c(F)c3)cc2n1] |
| 622 | 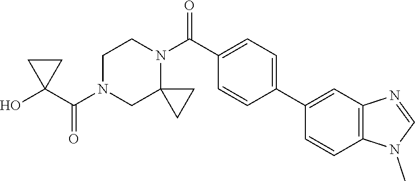 [SMILES: Cn1cnc2cc(-c3ccc(C(=O)N4CCN(C(=O)C5(O)CC5)CC45CC5)cc3)ccc21] |
| 623 | 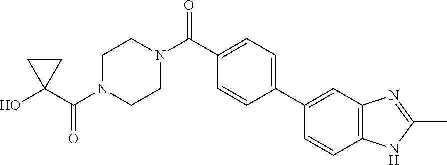 [SMILES: Cc1nc2cc(-c3ccc(C(=O)N4CCN(C(=O)C5(O)CC5)CC4)cc3)ccc2[nH]1] |
| 624 | 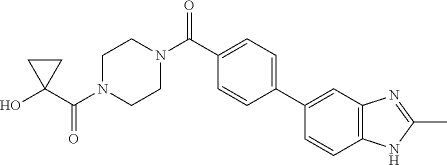 [SMILES: Cc1nc2cc(-c3ccc(C(=O)N4CCN(C(=O)C5(O)CC5)CC4)cc3)ccc2[nH]1] |
| 625 | 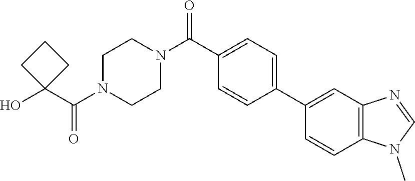 [SMILES: Cn1cnc2cc(-c3ccc(C(=O)N4CCN(C(=O)C5(O)CCC5)CC4)cc3)ccc21] |
| 626 | 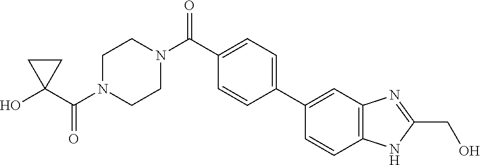 [SMILES: O=C(c1ccc(-c2ccc3[nH]c(CO)nc3c2)cc1)N1CCN(C(=O)C2(O)CC2)CC1] |
| 627 | 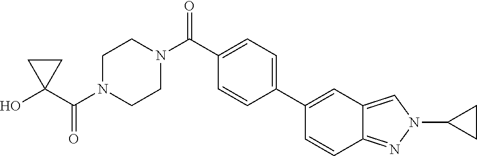 [SMILES: O=C(c1ccc(-c2ccc3nn(C4CC4)cc3c2)cc1)N1CCN(C(=O)C2(O)CC2)CC1] |
| 628 | 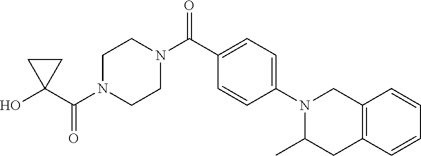 [SMILES: CC1Cc2ccccc2CN1c1ccc(C(=O)N2CCN(C(=O)C3(O)CC3)CC2)cc1] |
| 629 | 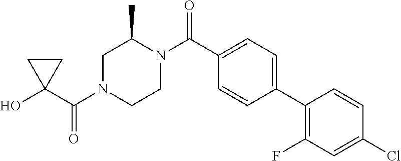 [SMILES: C[C@@H]1CN(C(=O)C2(O)CC2)CCN1C(=O)c1ccc(-c2ccc(Cl)cc2F)cc1] |
| 630 | 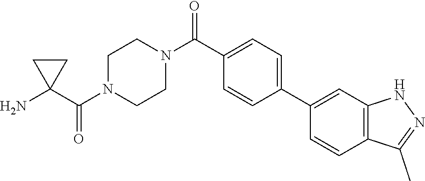 [SMILES: Cc1n[nH]c2cc(-c3ccc(C(=O)N4CCN(C(=O)C5(N)CC5)CC4)cc3)ccc12] |
| 631 | 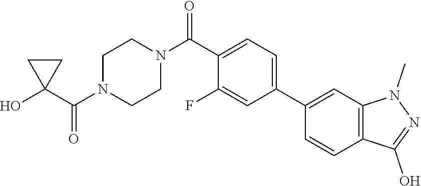 [SMILES: Cn1nc(O)c2ccc(-c3ccc(C(=O)N4CCN(C(=O)C5(O)CC5)CC4)c(F)c3)cc21] |
| 632 | 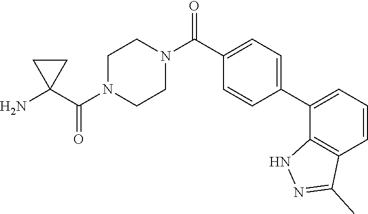 [SMILES: Cc1n[nH]c2c(-c3ccc(C(=O)N4CCN(C(=O)C5(N)CC5)CC4)cc3)cccc12] |
| 633 | 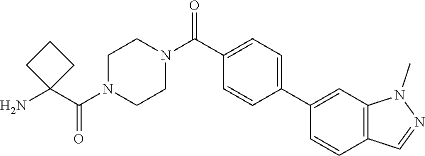 [SMILES: Cn1ncc2ccc(-c3ccc(C(=O)N4CCN(C(=O)C5(N)CCC5)CC4)cc3)cc21] |
| 634 | 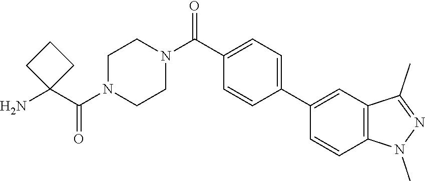 [SMILES: Cc1nn(C)c2ccc(-c3ccc(C(=O)N4CCN(C(=O)C5(N)CCC5)CC4)cc3)cc12] |
| 635 | 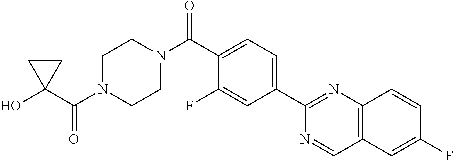 [SMILES: O=C(c1ccc(-c2ncc3cc(F)ccc3n2)cc1F)N1CCN(C(=O)C2(O)CC2)CC1] |
| 636 | 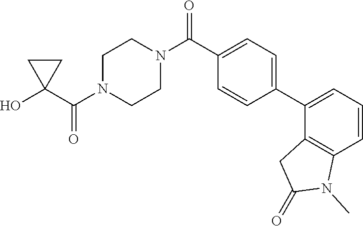 [SMILES: CN1C(=O)Cc2c(-c3ccc(C(=O)N4CCN(C(=O)C5(O)CC5)CC4)cc3)cccc21] |
| 637 | 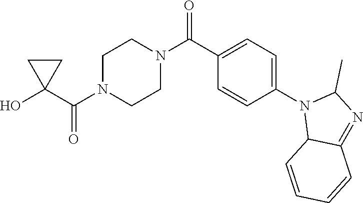 [SMILES: CC1N=C2C=CC=CC2N1c1ccc(C(=O)N2CCN(C(=O)C3(O)CC3)CC2)cc1] |
| 638 | 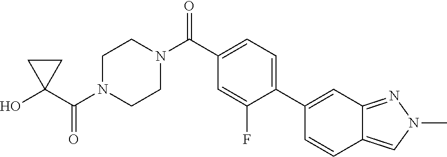 [SMILES: Cn1cc2ccc(-c3ccc(C(=O)N4CCN(C(=O)C5(O)CC5)CC4)cc3F)cc2n1] |
| 639 | 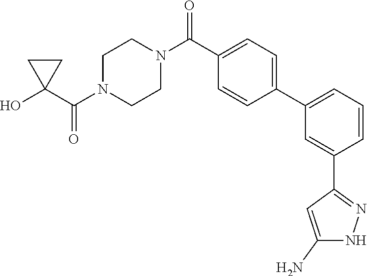 [SMILES: Nc1cc(-c2cccc(-c3ccc(C(=O)N4CCN(C(=O)C5(O)CC5)CC4)cc3)c2)n[nH]1] |
| 640 | 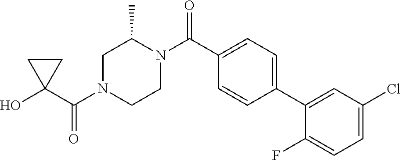 [SMILES: C[C@H]1CN(C(=O)C2(O)CC2)CCN1C(=O)c1ccc(-c2cc(Cl)ccc2F)cc1] |
| 641 | 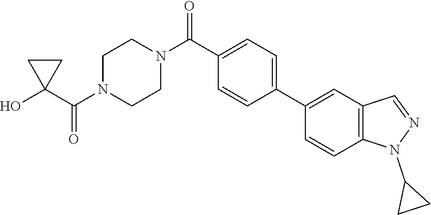 [SMILES: O=C(c1ccc(-c2ccc3c(cnn3C3CC3)c2)cc1)N1CCN(C(=O)C2(O)CC2)CC1] |
| 642 | 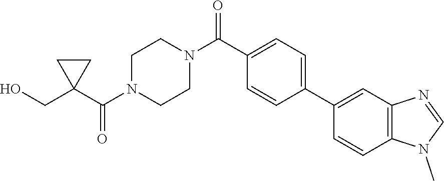 [SMILES: Cn1cnc2cc(-c3ccc(C(=O)N4CCN(C(=O)C5(CO)CC5)CC4)cc3)ccc21] |
| 643 | 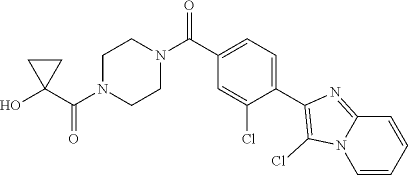 [SMILES: O=C(c1ccc(-c2nc3ccccn3c2Cl)c(Cl)c1)N1CCN(C(=O)C2(O)CC2)CC1] |
| 644 | 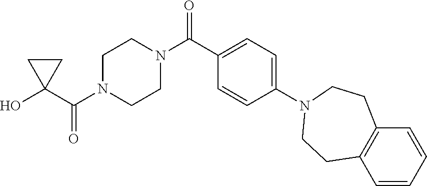 [SMILES: O=C(c1ccc(N2CCc3ccccc3CC2)cc1)N1CCN(C(=O)C2(O)CC2)CC1] |
| 645 | 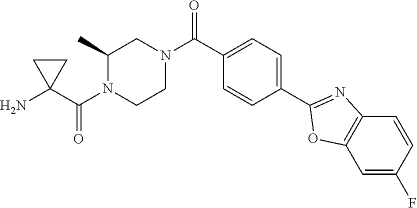 [SMILES: C[C@H]1CN(C(=O)c2ccc(-c3nc4ccc(F)cc4o3)cc2)CCN1C(=O)C1(N)CC1] |
| 646 | 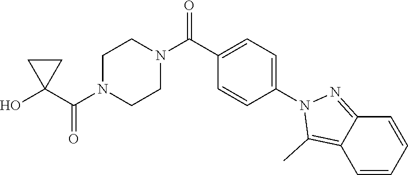 [SMILES: Cc1c2ccccc2nn1-c1ccc(C(=O)N2CCN(C(=O)C3(O)CC3)CC2)cc1] |
| 647 | 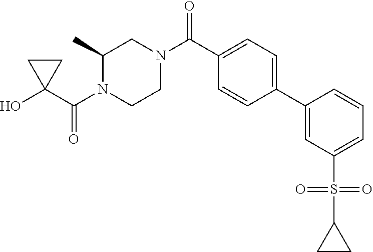 [SMILES: C[C@H]1CN(C(=O)c2ccc(-c3cccc(S(=O)(=O)C4CC4)c3)cc2)CCN1C(=O)C1(O)CC1] |
| 648 | 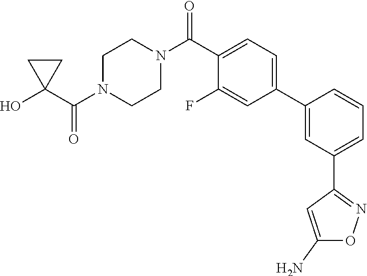 [SMILES: Nc1cc(-c2cccc(-c3ccc(C(=O)N4CCN(C(=O)C5(O)CC5)CC4)c(F)c3)c2)no1] |
| 649 | 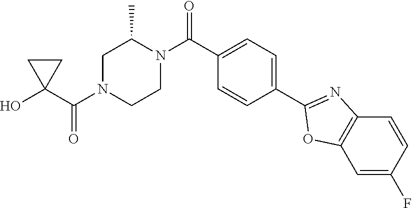 [SMILES: C[C@H]1CN(C(=O)C2(O)CC2)CCN1C(=O)c1ccc(-c2nc3ccc(F)cc3o2)cc1] |
| 650 | 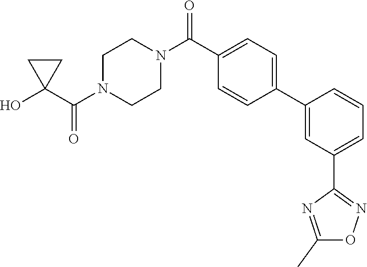 [SMILES: Cc1nc(-c2cccc(-c3ccc(C(=O)N4CCN(C(=O)C5(O)CC5)CC4)cc3)c2)no1] |
| 651 | 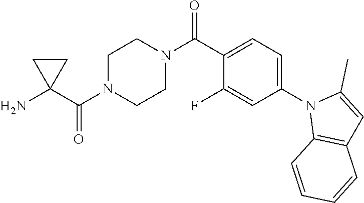 [SMILES: Cc1cc2ccccc2n1-c1ccc(C(=O)N2CCN(C(=O)C3(N)CC3)CC2)c(F)c1] |
| 652 | 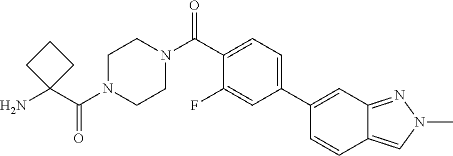 [SMILES: Cn1cc2ccc(-c3ccc(C(=O)N4CCN(C(=O)C5(N)CCC5)CC4)c(F)c3)cc2n1] |
| 653 | 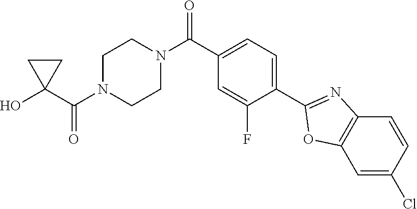 [SMILES: O=C(c1ccc(-c2nc3ccc(Cl)cc3o2)c(F)c1)N1CCN(C(=O)C2(O)CC2)CC1] |
| 654 | 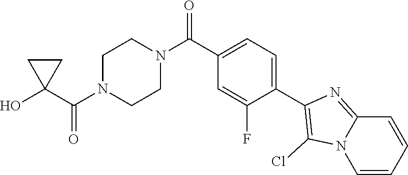 [SMILES: O=C(c1ccc(-c2nc3ccccn3c2Cl)c(F)c1)N1CCN(C(=O)C2(O)CC2)CC1] |
| 655 | 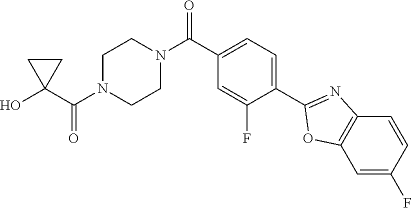 [SMILES: O=C(c1ccc(-c2nc3ccc(F)cc3o2)c(F)c1)N1CCN(C(=O)C2(O)CC2)CC1] |
| 656 | 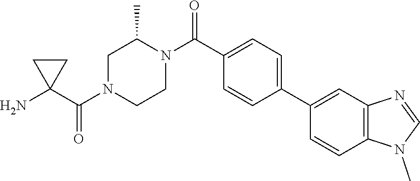 [SMILES: C[C@H]1CN(C(=O)C2(N)CC2)CCN1C(=O)c1ccc(-c2ccc3c(c2)ncn3C)cc1] |

In some embodiments, the FASN inhibitor is a compound of Formula (XIX):

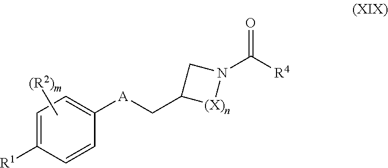

wherein: R¹ is phenyl, 5- or 6-membered heteroaryl, napthyl, 9- or 10-membered heterocyclyl; wherein said phenyl, 5- or 6-membered heteroaryl, napthyl, or 9- or 10-membered heterocyclyl is optionally substituted with from 1 to 3 substituents independently selected from halogen, C₁-C₄ alkyl, —CF₃, C₃-C₇ cycloalkyl, —C(O)C₁-C₄ alkyl, —C(O)C₃-C₇ cycloalkyl, —CO(phenyl), —C₁-C₄(═O)OH, —C(═O)O(C₁-C₄ alkyl), —CONR⁵R⁶, phenyl, —SO₂(C₁-C₄ alkyl), —SO₂NR⁵R⁶, cyano, oxo, hydroxyl, C₁-C₄ alkoxy, C₃-C₇ cycloalkoxy, hydroxyC₁-C₄ alkyl-, C₁-C₄ alkoxy C₁-C₄ alkyl-, —OCF₃, —NR⁵R⁶, R⁵R⁶NC₁-C₄ alkyl-, —NHC(O)C₁-C₄ alkyl, —NHCONR⁵R⁶, —NHSO₂C₁-C₄alkyl, —NHSO₂NR⁵R⁶, and R⁹; R⁵ is selected from the group consisting of hydrogen, C₁-C₄ alkyl, phenyl, and C₁-C₃alkylphenyl; R⁶ is hydrogen or C₁-C₄alkyl; or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or C₁-C₄ alkyl; R⁹ is a 5-membered heteroaryl ring containing 1 to 4 heteroatoms selected from oxygen, nitrogen, and sulfur, which is optionally substituted with 1 or 2 substituents selected from halogen, C₁-C₄ alkyl, C₁-C₄ alkoxy, and —NR⁵R⁶; each R² is independently selected from the group consisting of halogen, C₁-C₆alkyl, hydroxyl, CF₃, and C₁-C₄ alkoxy;
A is selected from

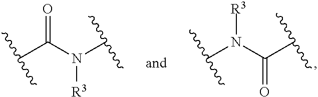

R³ is selected from the group consisting of C₁-C₄ alkyl, heteroaryl, C₁-C₄ alkyl 6-membered heteroaryl, and C₁-C₄alkylphenyl; R⁴ is selected from the group consisting of C₁-C₆alkyl, —CF₃, C₃-C₇cycloalkyl, C₁-C₄alkoxy, and —NR⁷R⁸; wherein C₃-C₇cycloalkyl is optionally substituted with 1 or 2 substituents independently selected from the group of halogen, C₁-C₄ alkyl, C₁-C₄ alkoxy, and —CONR⁷R⁸; R⁷ and R⁸ are each independently selected from hydrogen and C₁-C₄ alkyl, or R⁷ and R⁸ taken together with the nitrogen to which they are attached represent a 3- to 7-membered saturated ring optionally containing one other heteroatom selected from oxygen, nitrogen, and sulfur; m is 0, 1 or 2; n is 1 or 2; X is CH₂; or a pharmaceutically acceptable salt thereof.

In some embodiments, the FASN inhibitor is a compound of Formula (XIX-A):

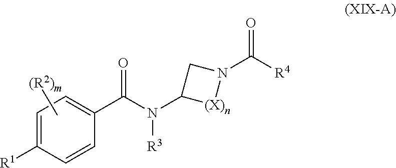

or a pharmaceutically acceptable salt thereof.

In some embodiments, the FASN inhibitor is a compound of Formula (XV-B):

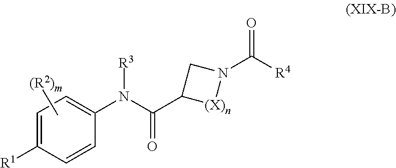

or a pharmaceutically acceptable salt thereof.

In some embodiments, R¹ is phenyl, 5- or 6-membered heteroaryl, napthyl, 9- or 10-membered heterocyclyl; wherein said phenyl, 5- or 6-membered heteroaryl, napthyl, or 9- or 10-membered heterocyclyl is optionally substituted with from 1 to 3 substituents independently selected from halogen, C₁-C₄ alkyl, —CF₃, C₃-C₇ cycloalkyl, —C(O)C₁-C₄ alkyl, —C(O)C₃-C₇ cycloalkyl, —CO(phenyl), —C₁-C₄(═O)OH, —C(═O)OC₁-C₄ alkyl, —CONR⁵R⁶, phenyl, —SO₂C₁-C₄ alkyl, —SO₂NR⁵R⁶, cyano, oxo, hydroxyl, C₁-C₄ alkoxy, C₃-C₇ cycloalkoxy, hydroxyC₁-C₄ alkyl-, C₁-C₄ alkoxy C₁-C₄ alkyl-OCF₃, —NR⁵R⁶, R⁵R⁶NC₁-C₄alkyl-, —NHC(O)C₁-C₄ alkyl, —NHCONR⁵R⁶, —NHSO₂C₁-C₄ alkyl, —NHSO₂NR⁵R⁶, and R⁹; R⁵ is selected from the group consisting of hydrogen, C₁-C₄ alkyl, phenyl, and C₁-C₃ alkylphenyl; R⁶ is hydrogen or C₁-C₄alkyl; or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or C₁-C₄alkyl; R⁹ is a 5-membered heteroaryl ring containing 1 to 4 heteroatoms selected from oxygen, nitrogen, and sulfur, which is optionally substituted with 1 or 2 substituents selected from halogen, C₁-C₄ alkyl, C₁-C₄ alkoxy, and —NR⁵R⁶; each R² is independently selected from the group consisting of halogen, C₁-C₆alkyl, hydroxyl, CF₃, and C₁-C₄ alkoxy; R³ is selected from the group consisting of C₁-C₄ alkyl, heteroaryl, C₁-C₄alkyl6-membered heteroaryl, and C₁-C₄ alkylphenyl; R⁴ is selected from the group consisting of C₁-C₆ alkyl, —CF₃, C₃-C₇ cycloalkyl, C₁-C₄ alkoxy, and —NR⁷R⁸; wherein C₃-C₇ cycloalkyl is optionally substituted with 1 or 2 substituents independently selected from the group of halogen, C₁-C₄ alkyl, C₁-C₄ alkoxy, and —CONR⁷R⁸; R⁷ and R⁸ are each independently selected from hydrogen and C₁-C₄ alkyl, or R⁷ and R⁸ taken together with the nitrogen to which they are attached represent a 3- to 7-membered saturated ring optionally containing one other heteroatom selected from oxygen, nitrogen, and sulfur; m is 0, 1 or 2; n is 1 or 2; X is CH₂; or a pharmaceutically acceptable salt thereof.

In other embodiments, R¹ is phenyl optionally substituted with from 1 to 3 substituents independently selected from halogen, C₁-C₄ alkyl, —CF₃, C₃-C₇ cycloalkyl, —C(O)C₁-C₄ alkyl, —C(O)C₃-C₇ cycloalkyl, —CO(phenyl), —C₁-C₄(═O)OH, —C(═O)OC₁-C₄ alkyl, —CONR⁵R⁶, phenyl, —SO₂C₁-C₄ alkyl, —SO₂NR⁵R⁶, cyano, oxo, hydroxyl, C₁-C₄alkoxy, C₃-C₇ cycloalkoxy, hydroxyC₁-C₄ alkyl-, C₁-C₄ alkoxy C₁-C₄ alkyl-, —OCF₃, —NR⁵R⁶, R⁵R⁶NC₁-C₄ alkyl-, —NHC(O)C₁-C₄ alkyl, —NHCONR⁵R⁶, —NHSO₂C₁-C₄alkyl, —NHSO₂NR⁵R⁶, and R⁹. In some embodiments, when present, R² is fluoro, hydroxyl, methyl, or methoxy. In other embodiments, R³ is C₁-C₄ alkyl, pyridinyl, pyrimidynyl, and C₁-C₄ alkylphenyl. In other embodiments, R⁴ is cyclopropyl. In some embodiments, R¹ is selected from the group of: furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, wherein each of said furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, and triazinyl is optionally substituted with from 1 to 3 substituents independently selected from halogen, C₁-C₄ alkyl, —CF₃, C₃-C₇ cycloalkyl, —C(O)C₁-C₄ alkyl, —C(O)C₃-C₇cycloalkyl, —C(O)phenyl, —C₁-C₄(═O)OH, —C(═O)C₁-C₄alkyl, —C(O)NR⁵R⁶, phenyl, —SO₂C₁-C₄ alkyl, —SO₂NR⁵R⁶, cyano, oxo, hydroxyl, C₁-C₄ alkoxy, C₃-C₇ cycloalkoxy, hydroxyC₁-C₄ alkyl-, C₁-C₄ alkoxy C₁-C₄ alkyl-, —OCF₃, —NR⁵R⁶, R⁵R⁶NC₁-C₄alkyl-, —NHC(O)C₁-C₄ alkyl, —NHCONR⁵R⁶, —NHSO₂C₁-C₄ alkyl, and —NHSO₂NR⁵R⁶. In some embodiments, R¹ is napthyl optionally substituted with from 1 to 3 substituents independently selected from halogen, C₁-C₄alkyl, —CF₃, C₃-C₇cycloalkyl, —C(O)C₁-C₄ alkyl, —C(O)C₃-C₇ cycloalkyl, —CO(phenyl), —C₁-C₄(═O)OH, —C(═O)C₁-C₄ alkyl, —CONR⁵R⁶, phenyl, —SO₂C₁-C₄ alkyl, —SO₂NR⁵R⁶, cyano, oxo, hydroxyl, C₁-C₄ alkoxy, C₃-C₇ cycloalkoxy, hydroxyC₁-C₄ alkyl-, C₁-C₄ alkoxy C₁-C₄ alkyl-, —OCF₃, —NR⁵R⁶, R⁵R⁶NC₁-C₄alkyl-, —NHC(O)C₁-C₄alkyl, —NHCONR⁵R⁶, —NHSO₂C₁-C₄alkyl, —NHSO₂NR⁵R⁶, and R⁹. In some embodiments, R¹ is selected from the group of benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, 1-H-indazolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, or pteridinyl, wherein said benzofuranyl, isobenzofuryl, 2,3-dihydrobenzofuryl, 1,3-benzodioxolyl, ihydrobenzodioxinyl, benzothienyl, indolizinyl, indolyl, isoindolyl, indolinyl, isoindolinyl, 1-H-indazolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl, each of which is optionally substituted with from 1 to 3 substituents independently selected from: halogen, C₁-C₄ alkyl, —CF₃, C₃-C₇ cycloalkyl, —C(O)C₁-C₄ alkyl, —C(O)C₃-C₇ cycloalkyl, —C(O)phenyl, —C₁-C₄(═O)OH, —C(═O)OC₁-C₄ alkyl, —C(O)NR⁵R⁶, phenyl, —SO₂C₁-C₄ alkyl, —SO₂NR⁵R⁶, cyano, oxo, hydroxyl, C₁-C₄ alkoxy, C₃-C₇ cycloalkoxy, hydroxyl C₁-C₄ alkyl-, C₁-C₄ alkoxy C₁-C₄ alkyl-, —OCF₃, —NR⁵R⁶, R⁵R⁶NC₁-C₄ alkyl-, —NHC(O)C₁-C₄ alkyl, —NHC(O)NR⁵R⁶, —NHSO₂C₁-C₄ alkyl, —NHSO₂NR⁵R⁶, and R⁹. In some embodiments, R² is fluoro, hydroxyl, methyl, or methoxy. In some embodiments, R³ is selected from C₁-C₄alkyl, pyridinyl, pyrimidynyl, and C₁-C₄alkylphenyl. In some embodiments, R⁴ is cyclopropyl. In some embodiments, R¹ is selected from the group of: phenyl, indolyl, benzofuranyl, indazolyl, benzoimidazolinyl, napthalyl, quinolyl, and wherein said phenyl is optionally substituted 1 to 3 times independently with a group selected from: methyloxy, cyano, NR⁵R⁶ and halogen, each R² is selected from the group consisting of halogen, C₁-C₆alkyl, hydroxyl, and C₁-C₄alkoxy; R³ is selected from the group consisting of C₁-C₄alkyl, pyridinyl, pyrimidynyl, phenyl and C₁-C₄alkylphenyl; and R⁴ is selected from the group consisting of C₁-C₆alkyl and cyclopropyl; m is 0, 1 or 2; n is 1 or 2; X is CH₂; or pharmaceutically acceptable salt thereof.

In some embodiments, the compound is one of the following:

| | Compound |
|---|---|
| 657 | 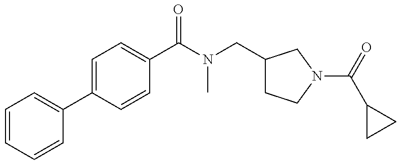 [SMILES: CN(CC1CCN(C(=O)C2CC2)C1)C(=O)c1ccc(-c2ccccc2)cc1] |
| 658 | 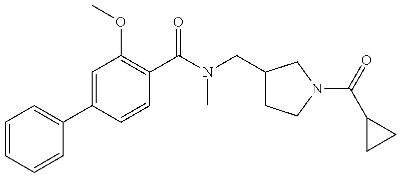 [SMILES: COc1cc(-c2ccccc2)ccc1C(=O)N(C)CC1CCN(C(=O)C2CC2)C1] |
| 659 | 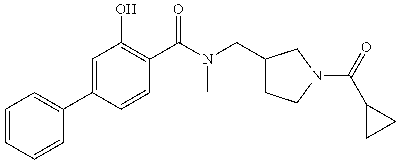 [SMILES: CN(CC1CCN(C(=O)C2CC2)C1)C(=O)c1ccc(-c2ccccc2)cc1O] |
| 660 | 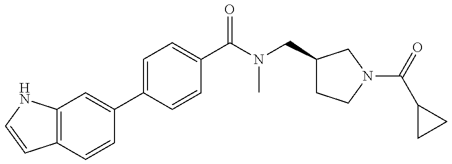 [SMILES: CN(C[C@@H]1CCN(C(=O)C2CC2)C1)C(=O)c1ccc(-c2ccc3cc[nH]c3c2)cc1] |
| 661 | 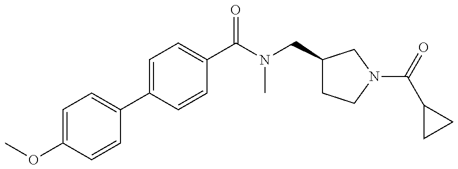 [SMILES: COc1ccc(-c2ccc(C(=O)N(C)C[C@@H]3CCN(C(=O)C4CC4)C3)cc2)cc1] |
| 662 | 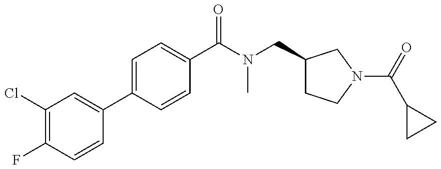 [SMILES: CN(C[C@@H]1CCN(C(=O)C2CC2)C1)C(=O)c1ccc(-c2ccc(F)c(Cl)c2)cc1] |
| 663 | 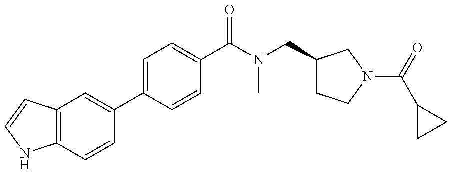 [SMILES: CN(C[C@@H]1CCN(C(=O)C2CC2)C1)C(=O)c1ccc(-c2ccc3[nH]ccc3c2)cc1] |
| 664 | 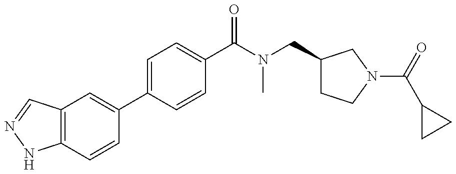 [SMILES: CN(C[C@@H]1CCN(C(=O)C2CC2)C1)C(=O)c1ccc(-c2ccc3[nH]ncc3c2)cc1] |
| 665 | 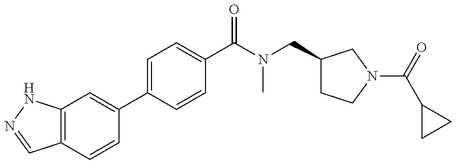 [SMILES: CN(C[C@@H]1CCN(C(=O)C2CC2)C1)C(=O)c1ccc(-c2ccc3cn[nH]c3c2)cc1] |
| 666 | 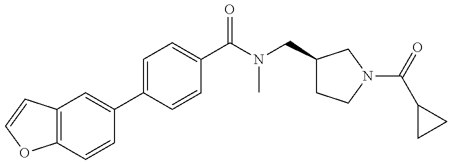 [SMILES: CN(C[C@@H]1CCN(C(=O)C2CC2)C1)C(=O)c1ccc(-c2ccc3occc3c2)cc1] |
| 667 | 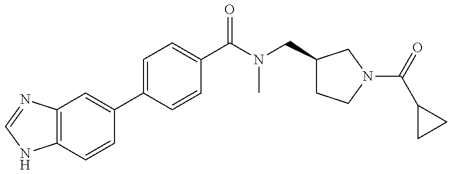 [SMILES: CN(C[C@@H]1CCN(C(=O)C2CC2)C1)C(=O)c1ccc(-c2ccc3[nH]cnc3c2)cc1] |
| 668 | 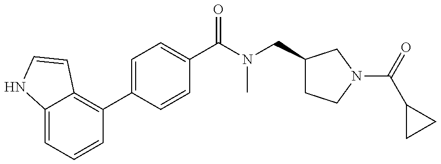 [SMILES: CN(C[C@@H]1CCN(C(=O)C2CC2)C1)C(=O)c1ccc(-c2cccc3[nH]ccc23)cc1] |
| 669 | 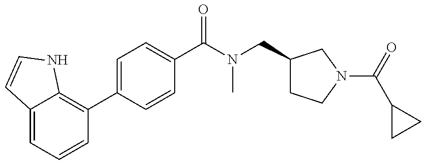 [SMILES: CN(C[C@@H]1CCN(C(=O)C2CC2)C1)C(=O)c1ccc(-c2cccc3cc[nH]c23)cc1] |
| 670 | 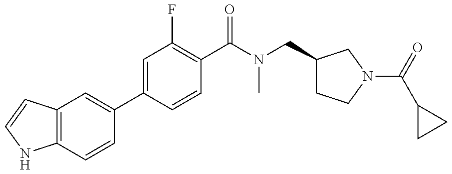 [SMILES: CN(C[C@@H]1CCN(C(=O)C2CC2)C1)C(=O)c1ccc(-c2ccc3[nH]ccc3c2)cc1F] |
| 671 | 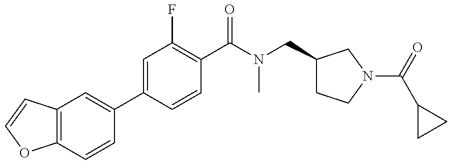 [SMILES: CN(C[C@@H]1CCN(C(=O)C2CC2)C1)C(=O)c1ccc(-c2ccc3occc3c2)cc1F] |
| 672 | 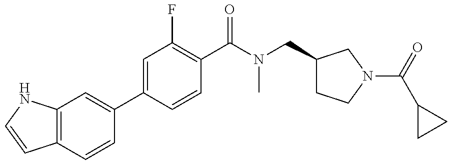 [SMILES: CN(C[C@@H]1CCN(C(=O)C2CC2)C1)C(=O)c1ccc(-c2ccc3cc[nH]c3c2)cc1F] |
| 673 | 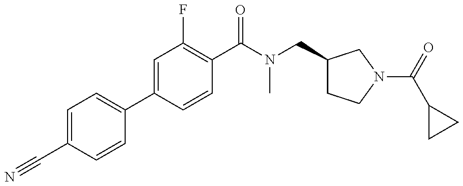 [SMILES: CN(C[C@@H]1CCN(C(=O)C2CC2)C1)C(=O)c1ccc(-c2ccc(C#N)cc2)cc1F] |
| 674 | 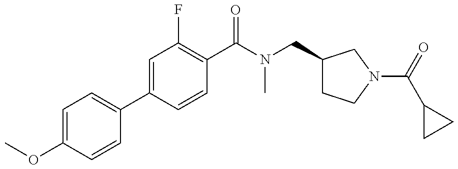 [SMILES: COc1ccc(-c2ccc(C(=O)N(C)C[C@@H]3CCN(C(=O)C4CC4)C3)c(F)c2)cc1] |
| 675 | 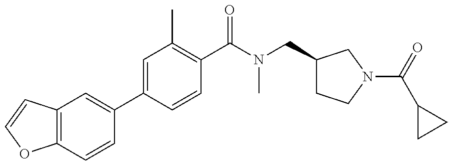 [SMILES: Cc1cc(-c2ccc3occc3c2)ccc1C(=O)N(C)C[C@@H]1CCN(C(=O)C2CC2)C1] |
| 676 | 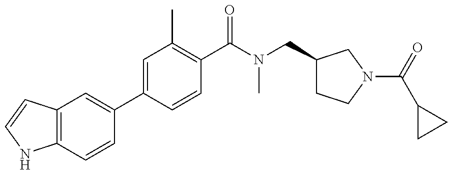 [SMILES: Cc1cc(-c2ccc3[nH]ccc3c2)ccc1C(=O)N(C)C[C@@H]1CCN(C(=O)C2CC2)C1] |
| 677 | 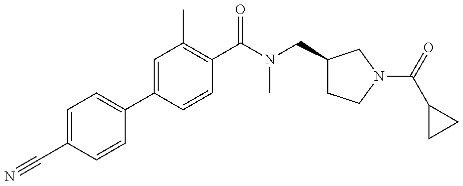 [SMILES: Cc1cc(-c2ccc(C#N)cc2)ccc1C(=O)N(C)C[C@@H]1CCN(C(=O)C2CC2)C1] |
| 678 | 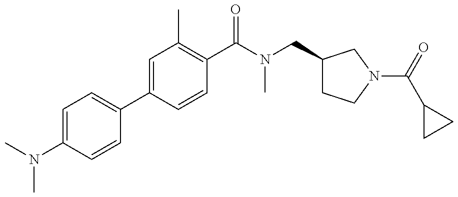 [SMILES: Cc1cc(-c2ccc(N(C)C)cc2)ccc1C(=O)N(C)C[C@@H]1CCN(C(=O)C2CC2)C1] |
| 679 | 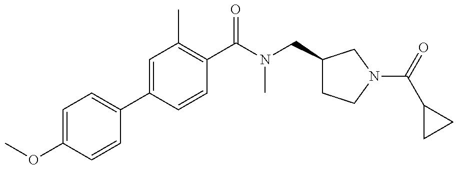 [SMILES: COc1ccc(-c2ccc(C(=O)N(C)C[C@@H]3CCN(C(=O)C4CC4)C3)c(C)c2)cc1] |
| 680 | 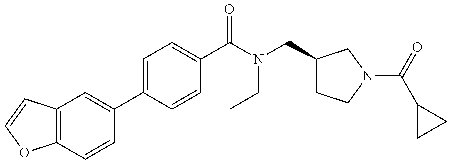 [SMILES: CCN(C[C@@H]1CCN(C(=O)C2CC2)C1)C(=O)c1ccc(-c2ccc3occc3c2)cc1] |
| 681 | 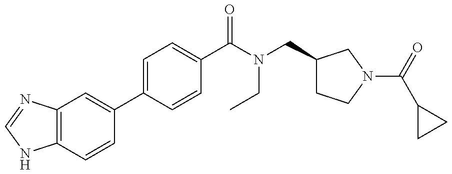 [SMILES: CCN(C[C@@H]1CCN(C(=O)C2CC2)C1)C(=O)c1ccc(-c2ccc3[nH]cnc3c2)cc1] |
| 682 | 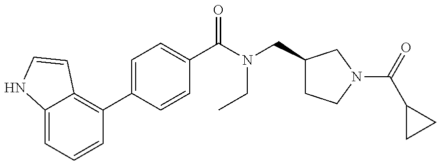 [SMILES: CCN(C[C@@H]1CCN(C(=O)C2CC2)C1)C(=O)c1ccc(-c2cccc3[nH]ccc23)cc1] |
| 683 | 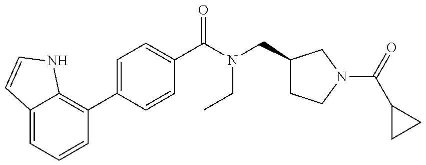 [SMILES: CCN(C[C@@H]1CCN(C(=O)C2CC2)C1)C(=O)c1ccc(-c2cccc3cc[nH]c23)cc1] |
| 684 | 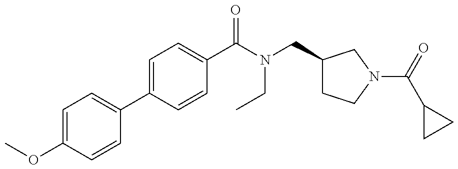 [SMILES: CCN(C[C@@H]1CCN(C(=O)C2CC2)C1)C(=O)c1ccc(-c2ccc(OC)cc2)cc1] |
| 685 | 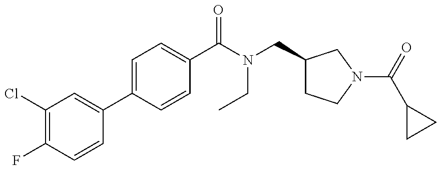 [SMILES: CCN(C[C@@H]1CCN(C(=O)C2CC2)C1)C(=O)c1ccc(-c2ccc(F)c(Cl)c2)cc1] |
| 686 | 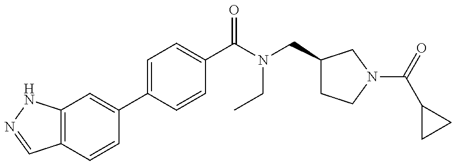 [SMILES: CCN(C[C@@H]1CCN(C(=O)C2CC2)C1)C(=O)c1ccc(-c2ccc3cn[nH]c3c2)cc1] |
| 687 | 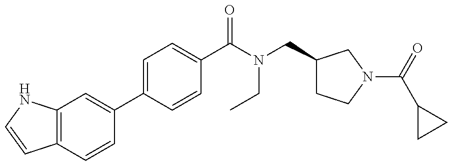 [SMILES: CCN(C[C@@H]1CCN(C(=O)C2CC2)C1)C(=O)c1ccc(-c2ccc3cc[nH]c3c2)cc1] |
| 688 | 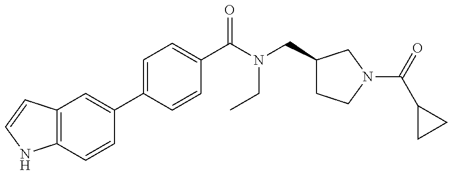 [SMILES: CCN(C[C@@H]1CCN(C(=O)C2CC2)C1)C(=O)c1ccc(-c2ccc3[nH]ccc3c2)cc1] |
| 689 | 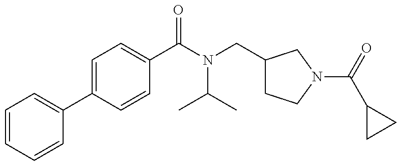 [SMILES: CC(C)N(CC1CCN(C(=O)C2CC2)C1)C(=O)c1ccc(-c2ccccc2)cc1] |
| 690 | 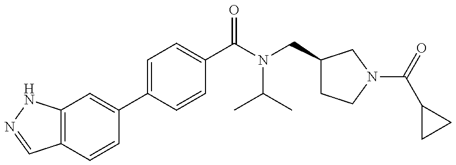 [SMILES: CC(C)N(C[C@@H]1CCN(C(=O)C2CC2)C1)C(=O)c1ccc(-c2ccc3cn[nH]c3c2)cc1] |
| 691 | 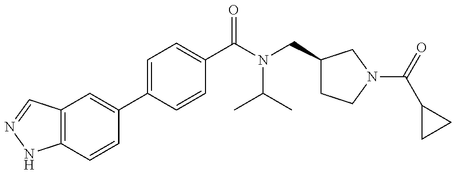 [SMILES: CC(C)N(C[C@@H]1CCN(C(=O)C2CC2)C1)C(=O)c1ccc(-c2ccc3[nH]ncc3c2)cc1] |
| 692 | 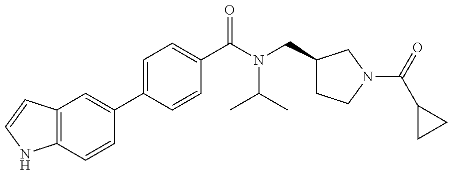 [SMILES: CC(C)N(C[C@@H]1CCN(C(=O)C2CC2)C1)C(=O)c1ccc(-c2ccc3[nH]ccc3c2)cc1] |
| 693 | 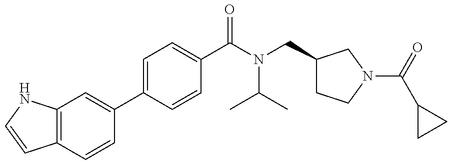 [SMILES: CC(C)N(C[C@@H]1CCN(C(=O)C2CC2)C1)C(=O)c1ccc(-c2ccc3cc[nH]c3c2)cc1] |
| 694 | 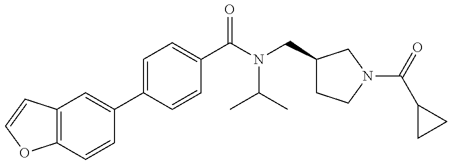 [SMILES: CC(C)N(C[C@@H]1CCN(C(=O)C2CC2)C1)C(=O)c1ccc(-c2ccc3occc3c2)cc1] |
| 695 | 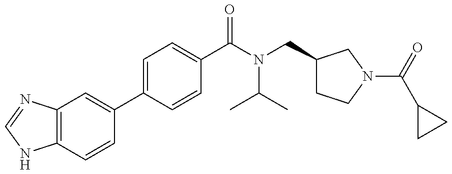 [SMILES: CC(C)N(C[C@@H]1CCN(C(=O)C2CC2)C1)C(=O)c1ccc(-c2ccc3[nH]cnc3c2)cc1] |
| 696 | 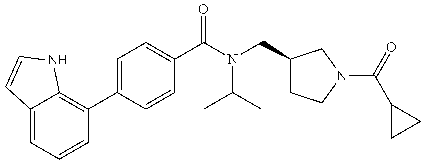 [SMILES: CC(C)N(C[C@@H]1CCN(C(=O)C2CC2)C1)C(=O)c1ccc(-c2cccc3cc[nH]c23)cc1] |
| 697 | 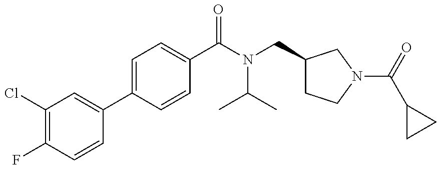 [SMILES: CC(C)N(C[C@@H]1CCN(C(=O)C2CC2)C1)C(=O)c1ccc(-c2ccc(F)c(Cl)c2)cc1] |
| 698 | 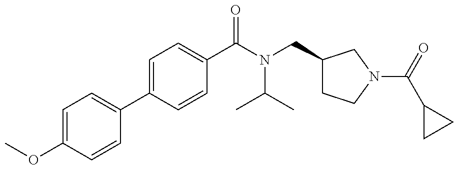 [SMILES: COc1ccc(-c2ccc(C(=O)N(C[C@@H]3CCN(C(=O)C4CC4)C3)C(C)C)cc2)cc1] |
| 699 | 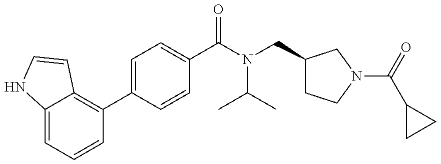 [SMILES: CC(C)N(C[C@@H]1CCN(C(=O)C2CC2)C1)C(=O)c1ccc(-c2cccc3[nH]ccc23)cc1] |
| 700 | 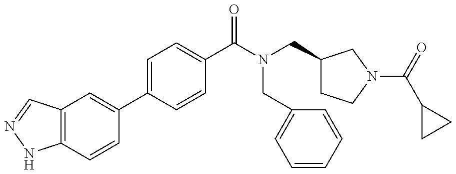 [SMILES: O=C(c1ccc(-c2ccc3[nH]ncc3c2)cc1)N(Cc1ccccc1)C[C@@H]1CCN(C(=O)C2CC2)C1] |
| 701 | 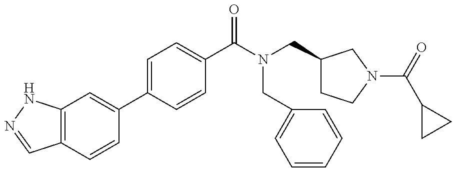 [SMILES: O=C(c1ccc(-c2ccc3cn[nH]c3c2)cc1)N(Cc1ccccc1)C[C@@H]1CCN(C(=O)C2CC2)C1] |
| 702 | 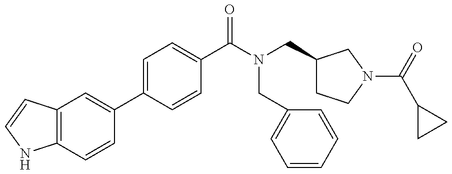 [SMILES: O=C(c1ccc(-c2ccc3[nH]ccc3c2)cc1)N(Cc1ccccc1)C[C@@H]1CCN(C(=O)C2CC2)C1] |
| 703 | 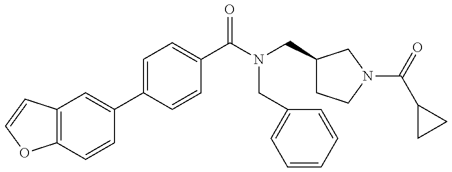 [SMILES: O=C(c1ccc(-c2ccc3occc3c2)cc1)N(Cc1ccccc1)C[C@@H]1CCN(C(=O)C2CC2)C1] |
| 704 | 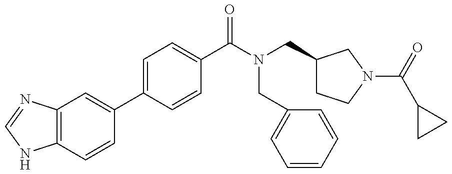 [SMILES: O=C(c1ccc(-c2ccc3[nH]cnc3c2)cc1)N(Cc1ccccc1)C[C@@H]1CCN(C(=O)C2CC2)C1] |
| 705 | 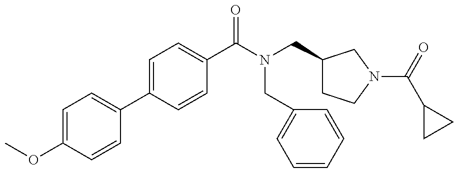 [SMILES: COc1ccc(-c2ccc(C(=O)N(Cc3ccccc3)C[C@@H]3CCN(C(=O)C4CC4)C3)cc2)cc1] |
| 706 | 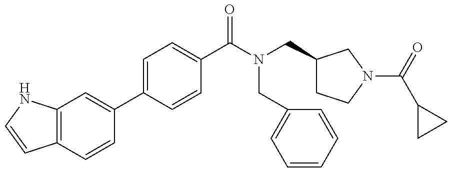 [SMILES: O=C(c1ccc(-c2ccc3cc[nH]c3c2)cc1)N(Cc1ccccc1)C[C@@H]1CCN(C(=O)C2CC2)C1] |
| 707 | 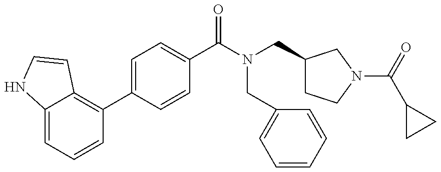 [SMILES: O=C(c1ccc(-c2cccc3[nH]ccc23)cc1)N(Cc1ccccc1)C[C@@H]1CCN(C(=O)C2CC2)C1] |
| 708 | 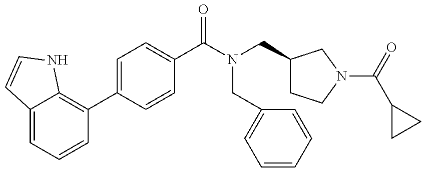 [SMILES: O=C(c1ccc(-c2cccc3cc[nH]c23)cc1)N(Cc1ccccc1)C[C@@H]1CCN(C(=O)C2CC2)C1] |
| 709 | 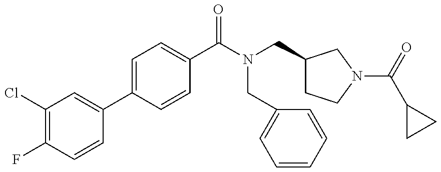 [SMILES: O=C(c1ccc(-c2ccc(F)c(Cl)c2)cc1)N(Cc1ccccc1)C[C@@H]1CCN(C(=O)C2CC2)C1] |
| 710 | 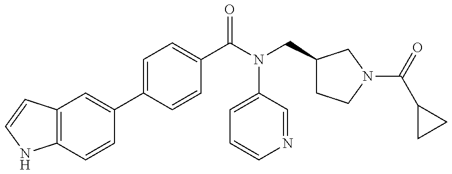 [SMILES: O=C(C1CC1)N1CC[C@@H](CN(C(=O)c2ccc(-c3ccc4[nH]ccc4c3)cc2)c2cccnc2)C1] |
| 711 | 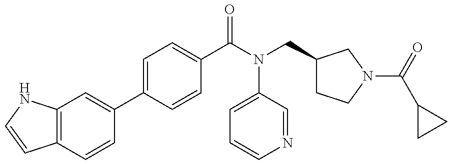 [SMILES: O=C(C1CC1)N1CC[C@@H](CN(C(=O)c2ccc(-c3ccc4cc[nH]c4c3)cc2)c2cccnc2)C1] |
| 712 | 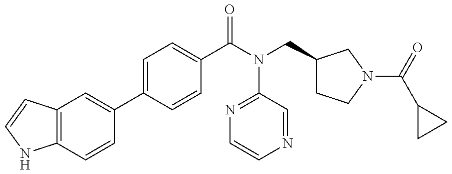 [SMILES: O=C(C1CC1)N1CC[C@@H](CN(C(=O)c2ccc(-c3ccc4[nH]ccc4c3)cc2)c2cnccn2)C1] |
| 713 | 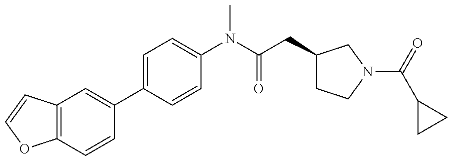 [SMILES: CN(C(=O)C[C@@H]1CCN(C(=O)C2CC2)C1)c1ccc(-c2ccc3occc3c2)cc1] |
| 714 | 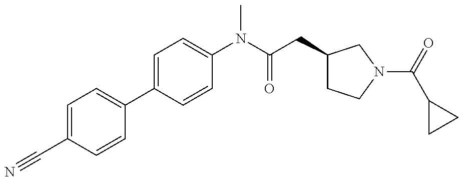 [SMILES: CN(C(=O)C[C@@H]1CCN(C(=O)C2CC2)C1)c1ccc(-c2ccc(C#N)cc2)cc1] |
| 715 | 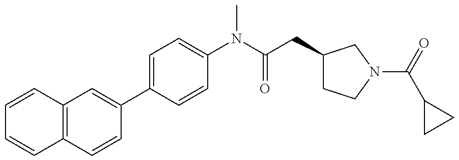 [SMILES: CN(C(=O)C[C@@H]1CCN(C(=O)C2CC2)C1)c1ccc(-c2ccc3ccccc3c2)cc1] |
| 716 | 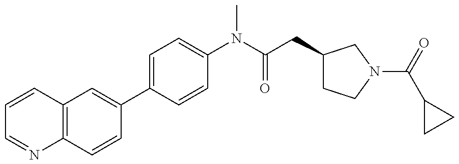 [SMILES: CN(C(=O)C[C@@H]1CCN(C(=O)C2CC2)C1)c1ccc(-c2ccc3ncccc3c2)cc1] |
| 717 | 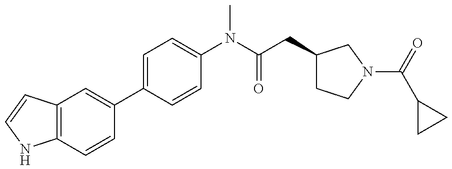 [SMILES: CN(C(=O)C[C@@H]1CCN(C(=O)C2CC2)C1)c1ccc(-c2ccc3[nH]ccc3c2)cc1] |
| 718 | 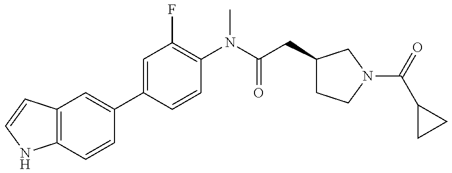 [SMILES: CN(C(=O)C[C@@H]1CCN(C(=O)C2CC2)C1)c1ccc(-c2ccc3[nH]ccc3c2)cc1F] |
| 719 | 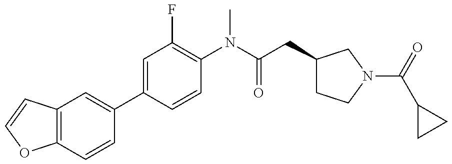 [SMILES: CN(C(=O)C[C@@H]1CCN(C(=O)C2CC2)C1)c1ccc(-c2ccc3occc3c2)cc1F] |
| 720 | 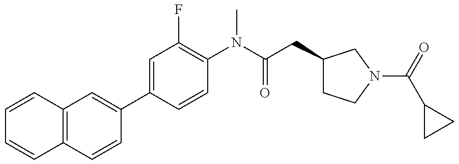 [SMILES: CN(C(=O)C[C@@H]1CCN(C(=O)C2CC2)C1)c1ccc(-c2ccc3ccccc3c2)cc1F] |
| 721 | 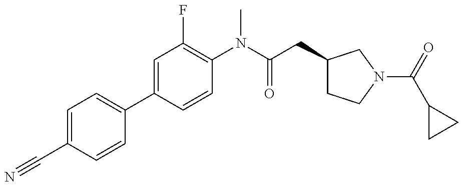 [SMILES: CN(C(=O)C[C@@H]1CCN(C(=O)C2CC2)C1)c1ccc(-c2ccc(C#N)cc2)cc1F] |
| 722 | 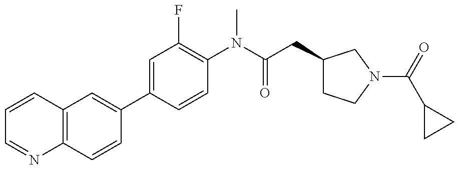 [SMILES: CN(C(=O)C[C@@H]1CCN(C(=O)C2CC2)C1)c1ccc(-c2ccc3ncccc3c2)cc1F] |
| 723 | 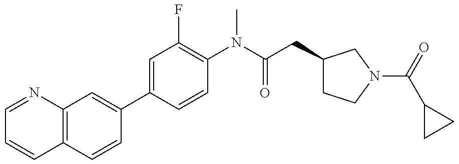 [SMILES: CN(C(=O)C[C@@H]1CCN(C(=O)C2CC2)C1)c1ccc(-c2ccc3cccnc3c2)cc1F] |
| 724 | 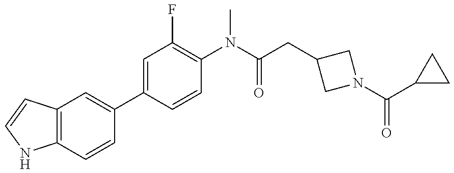 [SMILES: CN(C(=O)CC1CN(C(=O)C2CC2)C1)c1ccc(-c2ccc3[nH]ccc3c2)cc1F] |
| 725 | 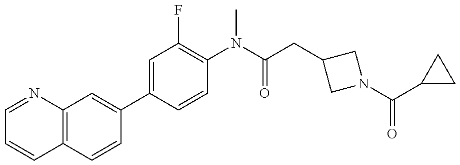 [SMILES: CN(C(=O)CC1CN(C(=O)C2CC2)C1)c1ccc(-c2ccc3cccnc3c2)cc1F] |

In some embodiments, the FASN inhibitor is a compound of Formula (XX):

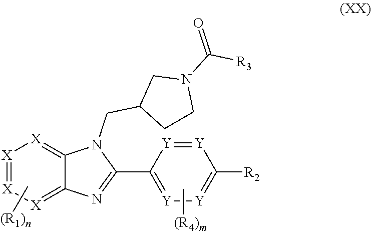

wherein, each R₁ is independently selected from the group consisting of: C₁₋₆ alkyl, alkoxy, hydroxyl, halogen, amino, substituted amino, alkylsulfonyl, cyano, heterocycloalkyl and —C(O)NRₐR_{b}, in which Rₐ and R_{b} are hydrogen, C₁₋₆ alkyl, C₃₋₇ cycloalkyl, or together Rₐ and R_{b} form a C₃₋₇ heterocycloalkyl; R² is selected from the group consisting of: aryl and heteroaryl, in which adjacent substituents in said aryl or heteroaryl together may form an additional five or six membered ring which contains 0-2 hetero atoms; R₃ is selected from the group consisting of: amino, alkylamino, dialkylamino, —OC₁₋₆ alkyl, C₁₋₆ alkyl and C₃₋₇cycloalkyl; R⁴ is selected from the group consisting of: C₁₋₆ alkyl, alkoxy, hydroxyl, and halogen; Y and X are C or N; n is 0-3; m is 0-4; or a pharmaceutically acceptable salt thereof; with the proviso that at least one but no more than two X's are N and at least two Y's are C.

In some embodiments, the FASN inhibitor is a compound of Formula (XX-A):

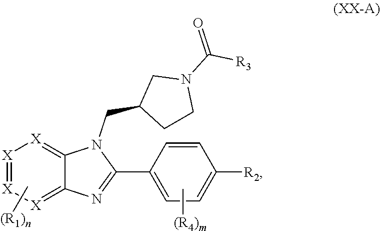

wherein, each R₁ is independently selected from the group consisting of: C₁₋₆ alkyl, alkoxy, hydroxyl, halogen, amino, alkylamino, dialkylamino, cyano, alkylsulfonyl, heterocycloalkyl and —C(O)NRₐR_{b}, in which Rₐ and R_{b} are hydrogen, C₁₋₆ alkyl, C₃₋₇cycloalkyl, or together Rₐ and R_{b} form a C₃₋₇ heterocycloalkyl; R² is selected from the group consisting of: aryl and heteroaryl, in which adjacent substituents in said aryl or heteroaryl together may form an additional five or six membered ring which contains 0-2 hetero atoms; R₃ is selected from the group consisting of: amino, alkylamino, dialkylamino, —OC₁₋₆ alkyl, C₁₋₆ alkyl and C₃₋₇ cycloalkyl; R⁴ is selected from the group consisting of: C₁₋₆ alkyl, alkoxy, hydroxyl, and halogen; X is C or N; n is 0-3; m is 0-4; or a pharmaceutically acceptable salt thereof; with the proviso that at least one but no more than two X's are N.

In some embodiments, R₃ is cyclopropyl. In some embodiments, n is 0-2 and m is 0. In some embodiments, n is 0-1 and m is 1. In some embodiments, R₁ is halogen, C₁₋₃ alkyl, amino, or alkylamino as defined above. In some embodiments, R₂ is heteroaryl. In some embodiments, R₂ is aryl. In some embodiments, R₂ is pyrrolopyridinyl, imidazopyridinyl, benzimidazolyl, benzothiazolyl, benzofuranyl or indolyl.

In some embodiments, the compound is 4′-(1-{[(3S)-1-(cyclopropylcarbonyl)-3-pyrrolidinyl]methyl}-1H-imidazo[4,5-b]pyridin-2-yl)-3-biphenylol, 1-{[(3S)-1-(cyclopropylcarbonyl)-3-pyrrolidinyl]methyl}-2-(3′-methyl-4-biphenylyl)-1H-imidazo[4,5-b]pyridine, 1-{[(3S)-1-(cyclopropylcarbonyl)-3-pyrrolidinyl]methyl}-2-(2′,4′-dimethyl-4-biphenylyl)-1H-imidazo[4,5-b]pyridine, 2-[2′-chloro-4′-(methyloxy)-4-biphenylyl]-1-{[(3S)-1-(cyclopropylcarbonyl)-3-pyrrolidinyl]methyl}-1H-imidazo[4,5-b]pyridine, 1-{[(3S)-1-(cyclopropylcarbonyl)-3-pyrrolidinyl]methyl}-2-[4-(1H-indazol-5-yl)phenyl]-1H-imidazo[4,5-b]pyridine, 1-{[(3S)-1-(cyclopropylcarbonyl)-3-pyrrolidinyl]methyl}-2-[4-(1H-indol-6-yl)phenyl]-1H-imidazo[4,5-b]pyridine, 1-{[(3S)-1-(cyclopropylcarbonyl)-3-pyrrolidinyl]methyl}-2-(4′-methyl-4-biphenylyl)-1H-imidazo[4,5-b]pyridine, 1-{[(3S)-1-(cyclopropylcarbonyl)-3-pyrrolidinyl]methyl}-2-(2′,4′-dichloro-4-biphenylyl)-1H-imidazo[4,5-b]pyridine, 1-{[(3S)-1-(cyclopropylcarbonyl)-3-pyrrolidinyl]methyl}-2-(4′-fluoro-3′-methyl-4-biphenylyl)-1H-imidazo[4,5-b]pyridine, 2-(4-biphenylyl)-1-{[(3S)-1-(cyclopropylcarbonyl)-3-pyrrolidinyl]methyl}-1H-imidazo[4,5-b]pyridine, 2-[4-(1-benzofuran-5-yl)phenyl]-1-{[(3S)-1-(cyclopropylcarbonyl)-3-pyrrolidinyl]methyl}-1H-imidazo[4,5-b]pyridine, 1-{[(3S)-1-(cyclopropylcarbonyl)-3-pyrrolidinyl]methyl}-2-(4′-fluoro-4-biphenylyl)-1H-imidazo[4,5-b]pyridine, 2-(4-biphenylyl)-1-{[1-(cyclopropylcarbonyl)-3-pyrrolidinyl]methyl}-1H-imidazo[4,5-c]pyridine, 1-{[1-(cyclopropylcarbonyl)-3-pyrrolidinyl]methyl}-2-[4-(1H-indazol-5-yl)phenyl]-1H-imidazo[4,5-c]pyridine, 1-{[(3S)-1-acetyl-3-pyrrolidinyl]methyl}-2-(4-biphenylyl)-1H-imidazo[4,5-c]pyridine, 2-(4-biphenylyl)-1-{[(3S)-1-propanoyl-3-pyrrolidinyl]methyl}-1H-imidazo[4,5-c]pyridine, 2-(4-biphenylyl)-1-{[(3S)-1-butanoyl-3-pyrrolidinyl]methyl}-1H-imidazo[4,5-c]pyridine, 2-(4-biphenylyl)-1-({(3S)-1-[(methyloxy)acetyl]-3-pyrrolidinyl}methyl)-1H-imidazo[4,5-c]pyridine, (3S)-3-{[2-(4-biphenylyl)-1H-imidazo[4,5-c]pyridin-1-yl]methyl}-N,N-dimethyl-1-pyrrolidinecarboxamide, (3S)-3-{[2-(4-biphenylyl)-1H-imidazo[4,5-c]pyridin-1-yl]methyl}-N-methyl-1-pyrrolidinecarboxamide, 2-(4-biphenylyl)-1-{[(3S)-1-(3,3,3-trifluoropropanoyl)-3-pyrrolidinyl]methyl}-1H-imidazo[4,5-c]pyridine, 2-[4-(1-benzofuran-5-yl)phenyl]-1-{[(3S)-1-(cyclopropylcarbonyl)-3-pyrrolidinyl]methyl}-1H-imidazo[4,5-c]pyridine, 1-{[(3S)-1-(cyclopropylcarbonyl)-3-pyrrolidinyl]methyl}-2-[4-(1H-indol-6-yl)phenyl]-1H-imidazo[4,5-c]pyridine, 1-{[(3S)-1-(cyclopropylcarbonyl)-3-pyrrolidinyl]methyl}-2-[4-(1H-indazol-6-yl)phenyl]-1H-imidazo[4,5-c]pyridine, 1-{[(3S)-1-(cyclopropylcarbonyl)-3-pyrrolidinyl]methyl}-2-[4-(1H-indol-5-yl)phenyl]-1H-imidazo[4,5-c]pyridine, 3-{[(3S)-1-(cyclopropylcarbonyl)-3-pyrrolidinyl]methyl}-2-(4′-fluoro-4-biphenylyl)-3H-imidazo[4,5-b]pyridine, 3-{[(3S)-1-(cyclopropylcarbonyl)-3-pyrrolidinyl]methyl}-2-[4′-(methyloxy)-4-biphenylyl]-3H-imidazo[4,5-b]pyridine, 3-{[(3S)-1-(cyclopropylcarbonyl)-3-pyrrolidinyl]methyl}-2-[4′-(ethyloxy)-4-biphenylyl]-3H-imidazo[4,5-b]pyridine, 3-{[(3S)-1-(cyclopropylcarbonyl)-3-pyrrolidinyl]methyl}-2-(2′,4′-dimethyl-4-biphenylyl)-3H-imidazo[4,5-b]pyridine, 4′-(3-{[(3S)-1-(cyclopropylcarbonyl)-3-pyrrolidinyl]methyl}-3H-imidazo[4,5-b]pyridin-2-yl)-3-biphenylcarboxylic acid, 2-(3′-chloro-4-biphenylyl)-3-{[(3S)-1-(cyclopropylcarbonyl)-3-pyrrolidinyl]methyl}-3H-imidazo[4,5-b]pyridine, 2-(4′-chloro-4-biphenylyl)-3-{[(3S)-1-(cyclopropylcarbonyl)-3-pyrrolidinyl]methyl}-3H-imidazo[4,5-b]pyridine, 2-(3′-chloro-4′-fluoro-4-biphenylyl)-3-{[(3S)-1-(cyclopropylcarbonyl)-3-pyrrolidinyl]methyl}-3H-imidazo[4,5-b]pyridine, 2-(4-biphenylyl)-3-{[(3S)-1-(cyclopropylcarbonyl)-3-pyrrolidinyl]methyl}-3H-imidazo[4,5-b]pyridine, 3-{[(3S)-1-(cyclopropylcarbonyl)-3-pyrrolidinyl]methyl}-2-[4-(1H-indol-6-yl)phenyl]-3H-imidazo[4,5-b]pyridine, 2-[4-(1-benzofuran-5-yl)phenyl]-3-{[(3S)-1-(cyclopropylcarbonyl)-3-pyrrolidinyl]methyl}-3H-imidazo[4,5-b]pyridine, 6-chloro-3-{[(3S)-1-(cyclopropylcarbonyl)-3-pyrrolidinyl]methyl}-2-(2′-methyl-4-biphenylyl)-3H-imidazo[4,5-b]pyridine, 6-chloro-3-{[(3S)-1-(cyclopropylcarbonyl)-3-pyrrolidinyl]methyl}-2-[3′-fluoro-4′-(methyloxy)-4-biphenylyl]-3H-imidazo[4,5-b]pyridine, 6-chloro-3-{[(3S)-1-(cyclopropylcarbonyl)-3-pyrrolidinyl]methyl}-2-(2′,4′-dimethyl-4-biphenylyl)-3H-imidazo[4,5-b]pyridine, 2-[4-(1-benzofuran-5-yl)phenyl]-6-chloro-3-{[(3S)-1-(cyclopropylcarbonyl)-3-pyrrolidinyl]methyl}-3H-imidazo[4,5-b]pyridine, 6-chloro-3-{[(3S)-1-(cyclopropylcarbonyl)-3-pyrrolidinyl]methyl}-2-[4-(1H-indazol-5-yl)phenyl]-3H-imidazo[4,5-b]pyridine, 2-[4-(1H-benzimidazol-5-yl)phenyl]-6-chloro-3-{[(3S)-1-(cyclopropylcarbonyl)-3-pyrrolidinyl]methyl}-3H-imidazo[4,5-b]pyridine, 6-chloro-3-{[(3S)-1-(cyclopropylcarbonyl)-3-pyrrolidinyl]methyl}-2-[4-(1H-indazol-6-yl)phenyl]-3H-imidazo[4,5-b]pyridine, 8-[4-(1-benzofuran-5-yl)phenyl]-6-chloro-9-{[(3S)-1-(cyclopropylcarbonyl)-3-pyrrolidinyl]methyl}-9H-purine, 6-chloro-9-{[(3S)-1-(cyclopropylcarbonyl)-3-pyrrolidinyl]methyl}-8-(2′,4′-dichloro-4-biphenylyl)-9H-purine, 8-(4-biphenylyl)-6-chloro-9-{[(3S)-1-(cyclopropylcarbonyl)-3-pyrrolidinyl]methyl}-9H-purine, 8-(4-biphenylyl)-9-{[(3S)-1-(cyclopropylcarbonyl)-3-pyrrolidinyl]methyl}-9H-purine, 8-[4-(1-benzofuran-5-yl)phenyl]-9-{[(3S)-1-(cyclopropylcarbonyl)-3-pyrrolidinyl]methyl}-9H-purine, 6-chloro-9-{[(3S)-1-(cyclopropylcarbonyl)-3-pyrrolidinyl]methyl}-8-(4′-fluoro-4-biphenylyl)-9H-purine, 9-{[(3S)-1-(cyclopropylcarbonyl)-3-pyrrolidinyl]methyl}-8-(4′-fluoro-4-biphenylyl)-6-(4-methyl-1-piperazinyl)-9H-purine, 1-{[(3S)-1-(cyclopropylcarbonyl)-3-pyrrolidinyl]methyl}-2-[4′-(methyloxy)-4-biphenylyl]-1H-imidazo[4,5-b]pyridine, 1-{[(3S)-1-(cyclopropylcarbonyl)-3-pyrrolidinyl]methyl}-2-[4-(1H-indol-5-yl)phenyl]-1H-imidazo[4,5-b]pyridine, 8-[4-(1-benzofuran-5-yl)phenyl]-9-{[(3S)-1-(cyclopropylcarbonyl)-3-pyrrolidinyl]methyl}-N-(1-methylethyl)-9H-purin-6-amine, 1-{[(3S)-1-(cyclopropylcarbonyl)-3-pyrrolidinyl]methyl}-2-[4-(1H-pyrrolo[2,3-b]pyridin-5-yl)phenyl]-1H-imidazo[4,5-c]pyridine, 1-{[(3S)-1-(cyclopropylcarbonyl)-3-pyrrolidinyl]methyl}-2-[4-(1H-indol-4-yl)phenyl]-1H-imidazo[4,5-c]pyridine, 1-{[(3S)-1-(cyclopropylcarbonyl)-3-pyrrolidinyl]methyl}-2-[4-(1H-indol-7-yl)phenyl]-1H-imidazo[4,5-c]pyridine, 1-{[(3S)-1-(cyclopropylcarbonyl)-3-pyrrolidinyl]methyl}-2-[4-(1H-pyrrolo[3,2-b]pyridin-6-yl)phenyl]-1H-imidazo[4,5-c]pyridine, 2-[4-(1,3-benzothiazol-5-yl)phenyl]-1-{[(3S)-1-(cyclopropylcarbonyl)-3-pyrrolidinyl]methyl}-1H-imidazo[4,5-c]pyridine, 1-{[(3S)-1-(cyclopropylcarbonyl)-3-pyrrolidinyl]methyl}-2-[4-(1H-indazol-4-yl)phenyl]-1H-imidazo[4,5-c]pyridine, 5-[4-(1-{[(3S)-1-(cyclopropylcarbonyl)-3-pyrrolidinyl]methyl}-1H-imidazo[4,5-c]pyridin-2-yl)phenyl]-1H-pyrazolo[3,4-b]pyridine, 2-[4-(1H-benzimidazol-5-yl)phenyl]-1-{[(3S)-1-(cyclopropylcarbonyl)-3-pyrrolidinyl]methyl}-1H-imidazo[4,5-c]pyridine, 1-{[(3S)-1-(cyclopropylcarbonyl)-3-pyrrolidinyl]methyl}-2-[4-(1H-pyrrolo[2,3-b]pyridin-4-yl)phenyl]-1H-imidazo[4,5-c]pyridine, 1-{[(3S)-1-(cyclopropylcarbonyl)-3-pyrrolidinyl]methyl}-2-(4-imidazo[1,2-a]pyridin-7-ylphenyl)-1H-imidazo[4,5-c]pyridine, 3-{[(3S)-1-(cyclopropylcarbonyl)-3-pyrrolidinyl]methyl}-2-[4-(1H-indol-6-yl)phenyl]-6-methyl-3H-imidazo[4,5-b]pyridine, or 3-{[(3S)-1-(cyclopropylcarbonyl)-3-pyrrolidinyl]methyl}-2-[4-(1H-indol-6-yl)phenyl]-5-(methyloxy)-3H-imidazo[4,5-b]pyridine.

In some embodiments, the FASN inhibitor is a compound of Formula (XXI):

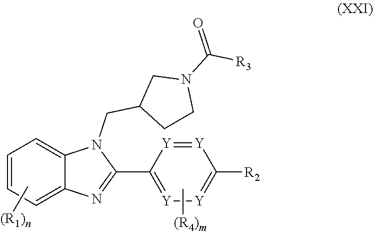

wherein, each R₁ is independently selected from the group consisting of: halogen, C₁₋₆ alkyl, alkoxy, hydroxyl, amino, substituted amino, alkylsulfonyl, C₄₋₇ heterocycloalkyl, cyano, and —C(O)NRₐR_{b}, in which Rₐ and R_{b} are hydrogen, C₁₋₆ alkyl, C₃₋₇ cycloalkyl, or together Rₐ and R_{b} form a C₄₋₇ heterocycloalkyl; R₂ is selected from the group consisting of: optionally substituted aryl and heteroaryl, in which adjacent substituents together may form an additional five or six membered ring which contains 0-2 hetero atoms; R₃ is selected from the group consisting of: amino, alkylamino, dialkylamino, —OC₁₋₆ alkyl, C₁₋₆ alkyl and C₃₋₇ cycloalkyl; R₄ is selected from the group consisting of: C₁₋₆ alkyl, alkoxy, hydroxyl and halogen; Y is C or N; and n is 0-4; m is 0-4; or a pharmaceutically acceptable salt thereof; with the proviso that at least two Y's are C.

In some embodiments, the FASN inhibitor is a compound of Formula (XXI-A):

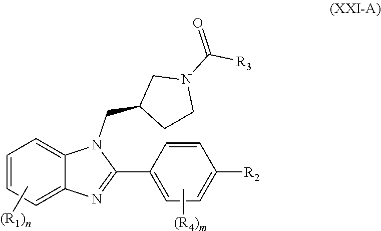

wherein, each R₁ is independently selected from the group consisting of: C₁₋₆ alkyl, alkoxy, cyano, halogen, and —C(O)NRₐR_{b}, in which Rₐ and R_{b} are hydrogen, C₁₋₆ alkyl, C₃₋₇ cycloalkyl, or together Rₐ and R_{b} form a C₄₋₇ heterocycloalkyl; R₂ is selected from the group consisting of: optionally substituted aryl and heteroaryl, in which adjacent substituents together may form an additional five or six membered ring which contains 0-2 hetero atoms; R₃ is selected from the group consisting of: amino, alkylamino, dialkylamino, —OC₁₋₆ alkyl, C₁₋₆ alkyl and C₃₋₇ cycloalkyl; R₄ is selected from the group consisting of: C₁₋₆ alkyl, alkoxy, hydroxyl and halogen; and n is 0-4 m is 0-4; or a pharmaceutically acceptable salt thereof.

In some embodiments, R3 is cyclopropyl. In some embodiments, n is 0-2 and m is 0. In some embodiments, n is 1 and m is 0. In some embodiments, R₁ is halogen, cyano, alkoxy, C₁₋₃ alkyl, or —C(O)NRₐR_{b} as defined above. In some embodiments, R₂ is heteroaryl. In some embodiments, R₂ is aryl. In some embodiments, R₂ is an aryl or heteroaryl selected from the group consisting of: indole, phenyl, indazole, benzofuranyl, wherein said aryl or heteroaryl may be substituted by one to three groups selected from: alkyl, halogen, hydroxyl, —SO₂Me and alkoxy.

In some embodiments, the compound is 1-{[(3S)-1-(cyclopropylcarbonyl)-3-pyrrolidinyl]methyl}-2-[4-(1H-indol-5-yl)phenyl]-1H-benzimidazole, 1-{[(3S)-1-(cyclopropylcarbonyl)-3-pyrrolidinyl]methyl}-2-[4-(1H-indol-6-yl)phenyl]-1H-benzimidazole, 2-[4-(1-Benzofuran-5-yl)phenyl]-1-{[(3S)-1-(cyclopropylcarbonyl)-3-pyrrolidinyl]methyl}-1H-benzimidazole, 6-[4-(1-{[(3S)-1-(cyclopropylcarbonyl)-3-pyrrolidinyl]methyl}-1H-benzimidazol-2-yl)phenyl]-1,3-benzothiazole, 1-{[(3S)-1-(cyclopropylcarbonyl)-3-pyrrolidinyl]methyl}-2-(4′-fluoro-4-biphenylyl)-4-(methyloxy)-1H-benzimidazole, 1-{[(3S)-1-(cyclopropylcarbonyl)-3-pyrrolidinyl]methyl}-4-(methyloxy)-2-[4′-(methyloxy)-4-biphenylyl]-1H-benzimidazole, 1-{[(3S)-1-(cyclopropylcarbonyl)-3-pyrrolidinyl]methyl}-2-[4-(1H-indol-5-yl)phenyl]-4-(methyloxy)-1H-benzimidazole, 1-{[(3S)-1-(cyclopropylcarbonyl)-3-pyrrolidinyl]methyl}-2-[4-(1H-indol-6-yl)phenyl]-4-(methyloxy)-1H-benzimidazole, 5-{4-[1-{[(3S)-1-(cyclopropylcarbonyl)-3-pyrrolidinyl]methyl}-4-(methyloxy)-1H-benzimidazol-2-yl]phenyl}-1H-indazole, 6-{4-[1-{[(3S)-1-(cyclopropylcarbonyl)-3-pyrrolidinyl]methyl}-4-(methyloxy)-1H-benzimidazol-2-yl]phenyl}-1H-indazole, 2-(4-Biphenylyl)-1-{[(3S)-1-(cyclopropylcarbonyl)-3-pyrrolidinyl]methyl}-4-(methyloxy)-1H-benzimidazole, 2-[4-(1-Benzofuran-5-yl)phenyl]-1-{[(3S)-1-(cyclopropylcarbonyl)-3-pyrrolidinyl]methyl}-4-(methyloxy)-1H-benzimidazole, 1-{[(3S)-1-(cyclopropylcarbonyl)-3-pyrrolidinyl]methyl}-2-(4′-fluoro-4-biphenylyl)-4-methyl-1H-benzimidazole, 1-{[(3S)-1-(cyclopropylcarbonyl)-3-pyrrolidinyl]methyl}-4-methyl-2-[4′-(methyloxy)-4-biphenylyl]-1H-benzimidazole, 2-[4-(1-Benzofuran-5-yl)phenyl]-1-{[(3S)-1-(cyclopropylcarbonyl)-3-pyrrolidinyl]methyl}-4-methyl-1H-benzimidazole, 1-{[(3S)-1-(cyclopropylcarbonyl)-3-pyrrolidinyl]methyl}-2-[4-(1H-indol-5-yl)phenyl]-4-methyl-1H-benzimidazole, 1-{[(3S)-1-(cyclopropylcarbonyl)-3-pyrrolidinyl]methyl}-2-[4-(1H-indol-6-yl)phenyl]-4-methyl-1H-benzimidazole, 5-[4-(1-{[(3S)-1-(cyclopropylcarbonyl)-3-pyrrolidinyl]methyl}-4-methyl-1H-benzimidazol-2-yl)phenyl]-1H-indazole, 6-[4-(1-{[(3S)-1-(cyclopropylcarbonyl)-3-pyrrolidinyl]methyl}-4-methyl-1H-benzimidazol-2-yl)phenyl]-1H-indazole, 2-(4-Biphenylyl)-1-{[(3S)-1-(cyclopropylcarbonyl)-3-pyrrolidinyl]methyl}-4-methyl-1H-benzimidazole, 1-{[(3S)-1-(cyclopropylcarbonyl)-3-pyrrolidinyl]methyl}-2-(4′-fluoro-4-biphenylyl)-4-(trifluoromethyl)-1H-benzimidazole, 1-{[(3S)-1-(cyclopropylcarbonyl)-3-pyrrolidinyl]methyl}-2-[4′-(methyloxy)-4-biphenylyl]-4-(trifluoromethyl)-1H-benzimidazole, 1-{[(3S)-1-(cyclopropylcarbonyl)-3-pyrrolidinyl]methyl}-2-[4-(1H-indol-5-yl)phenyl]-4-(trifluoromethyl)-1H-benzimidazole, 1-{[(3S)-1-(cyclopropylcarbonyl)-3-pyrrolidinyl]methyl}-2-[4-(1H-indol-6-yl)phenyl]-4-(trifluoromethyl)-1H-benzimidazole, 5-{4-[1-{[(3S)-1-(cyclopropylcarbonyl)-3-pyrrolidinyl]methyl}-4-(trifluoromethyl)-1H-benzimidazol-2-yl]phenyl}-1H-indazole, 6-{4-[1-{[(3S)-1-(cyclopropylcarbonyl)-3-pyrrolidinyl]methyl}-4-(trifluoromethyl)-1H-benzimidazol-2-yl]phenyl}-1H-indazole, 2-[4-(1-Benzofuran-5-yl)phenyl]-1-{[(3S)-1-(cyclopropylcarbonyl)-3-pyrrolidinyl]methyl}-4-(trifluoromethyl)-1H-benzimidazole, 2-(4-Biphenylyl)-1-{[(3S)-1-(cyclopropylcarbonyl)-3-pyrrolidinyl]methyl}-4-(trifluoromethyl)-1H-benzimidazole, 4-Bromo-1-{[(3S)-1-(cyclopropylcarbonyl)-3-pyrrolidinyl]methyl}-2-(4′-fluoro-4-biphenylyl)-1H-benzimidazole, 4-Bromo-1-{[(3S)-1-(cyclopropylcarbonyl)-3-pyrrolidinyl]methyl}-2-[4′-(methyloxy)-4-biphenylyl]-1H-benzimidazole, 4-Bromo-1-{[(3S)-1-(cyclopropylcarbonyl)-3-pyrrolidinyl]methyl}-2-[4-(1H-indol-5-yl)phenyl]-1H-benzimidazole, 5-[4-(4-Bromo-1-{[(3S)-1-(cyclopropylcarbonyl)-3-pyrrolidinyl]methyl}-1H-benzimidazol-2-yl)phenyl]-1H-indazole, 2-(4-Biphenylyl)-4-bromo-1-{[(3S)-1-(cyclopropylcarbonyl)-3-pyrrolidinyl]methyl}-1H-benzimidazole, 4-Bromo-1-{[(3S)-1-(cyclopropylcarbonyl)-3-pyrrolidinyl]methyl}-2-[4-(1H-indol-6-yl)phenyl]-1H-benzimidazole, 6-[4-(4-Bromo-1-{[(3S)-1-(cyclopropylcarbonyl)-3-pyrrolidinyl]methyl}-1H-benzimidazol-2-yl)phenyl]-1H-indazole, 2-[4-(1-Benzofuran-5-yl)phenyl]-4-bromo-1-{[(3S)-1-(cyclopropylcarbonyl)-3-pyrrolidinyl]methyl}-1H-benzimidazole, 2-[4-(1-Benzofuran-5-yl)phenyl]-1-{[(3S)-1-(cyclopropylcarbonyl)-3-pyrrolidinyl]methy 1}-5-(methyloxy)-1H-benzimidazole, 1-{[(3S)-1-(cyclopropylcarbonyl)-3-pyrrolidinyl]methyl}-2-[4-(1H-indol-5-yl)phenyl]-5-(methyloxy)-1H-benzimidazole, 1-{[(3S)-1-(cyclopropylcarbonyl)-3-pyrrolidinyl]methyl}-2-[4-(1H-indol-6-yl)phenyl]-5-(methyloxy)-1H-benzimidazole, 5-{4-[1-{[(3S)-1-(cyclopropylcarbonyl)-3-pyrrolidinyl]methyl}-5-(methyloxy)-1H-benzimidazol-2-yl]phenyl}-1H-indazole, 6-{4-[1-{[(3S)-1-(cyclopropylcarbonyl)-3-pyrrolidinyl]methyl}-5-(methyloxy)-1H-benzimidazol-2-yl]phenyl}-1H-indazole, 2-(4-Biphenylyl)-1-{[(3S)-1-(cyclopropylcarbonyl)-3-pyrrolidinyl]methyl}-5-(methyloxy)-1H-benzimidazole, 1-{[(3S)-1-(cyclopropylcarbonyl)-3-pyrrolidinyl]methyl}-2-(4′-fluoro-4-biphenylyl)-5-(methyloxy)-1H-benzimidazole, 1-{[(3S)-1-(cyclopropylcarbonyl)-3-pyrrolidinyl]methyl}-5-(methyloxy)-2-[4′-(methyloxy)-4-biphenylyl]-1H-benzimidazole, 1-{[(3S)-1-(cyclopropylcarbonyl)-3-pyrrolidinyl]methyl}-2-(4′-fluoro-4-biphenylyl)-5-methyl-1H-benzimidazole, 1-{[(3S)-1-(cyclopropylcarbonyl)-3-pyrrolidinyl]methyl}-5-methyl-2-[4′-(methyloxy)-4-biphenylyl]-1H-benzimidazole, 1-{[(3S)-1-(cyclopropylcarbonyl)-3-pyrrolidinyl]methyl}-2-[4-(1H-indol-5-yl)phenyl]-5-methyl-1H-benzimidazole, 1-{[(3S)-1-(cyclopropylcarbonyl)-3-pyrrolidinyl]methyl}-2-[4-(1H-indol-6-yl)phenyl]-5-methyl-1H-benzimidazole, 5-[4-(1-{[(3S)-1-(cyclopropylcarbonyl)-3-pyrrolidinyl]methyl}-5-methyl-1H-benzimidazol-2-yl)phenyl]-1H-indazole, 6-[4-(1-{[(3S)-1-(cyclopropylcarbonyl)-3-pyrrolidinyl]methyl}-5-methyl-1H-benzimidazol-2-yl)phenyl]-1H-indazole, 2-(4-Biphenylyl)-1-{[(3S)-1-(cyclopropylcarbonyl)-3-pyrrolidinyl]methyl}-5-methyl-1H-benzimidazole, 2-[4-(1-Benzofuran-5-yl)phenyl]-1-{[(3S)-1-(cyclopropylcarbonyl)-3-pyrrolidinyl]methyl}-5-methyl-1H-benzimidazole, 5-{4-[1-{[(3S)-1-(cyclopropylcarbonyl)-3-pyrrolidinyl]methyl}-5-(trifluoromethyl)-1H-benzimidazol-2-yl]phenyl}-1H-indazole, 1-{[(3S)-1-(cyclopropylcarbonyl)-3-pyrrolidinyl]methyl}-2-[4-(1H-indol-6-yl)phenyl]-5-(trifluoromethyl)-1H-benzimidazole, 6-{4-[1-{[(3S)-1-(cyclopropylcarbonyl)-3-pyrrolidinyl]methyl}-5-(trifluoromethyl)-1H-benzimidazol-2-yl]phenyl}-1H-indazole, 2-(4-biphenylyl)-1-{[(3S)-1-(cyclopropylcarbonyl)-3-pyrrolidinyl]methyl}-5-(trifluoromethyl)-1H-benzimidazole, 2-[4-(1-Benzofuran-5-yl)phenyl]-1-{[(3S)-1-(cyclopropylcarbonyl)-3-pyrrolidinyl]methyl}-5-(trifluoromethyl)-1H-benzimidazole, 4′-[1-{[(3S)-1-(cyclopropylcarbonyl)-3-pyrrolidinyl]methyl}-5-(trifluoromethyl)-1H-benzimidazol-2-yl]-3-biphenylol, 1-{[(3S)-1-(cyclopropylcarbonyl)-3-pyrrolidinyl]methyl}-2-(4′-fluoro-4-biphenylyl)-5-(trifluoromethyl)-1H-benzimidazole, 1-{[(3S)-1-(cyclopropylcarbonyl)-3-pyrrolidinyl]methyl}-2-[4′-(methyloxy)-4-biphenylyl]-5-(trifluoromethyl)-1H-benzimidazole, 2-(3′-Chloro-4-biphenylyl)-1-{[(3S)-1-(cyclopropylcarbonyl)-3-pyrrolidinyl]methyl}-5-(trifluoromethyl)-1H-benzimidazole, 1-{[(3S)-1-(cyclopropylcarbonyl)-3-pyrrolidinyl]methyl}-2-[4-(1H-indol-5-yl)phenyl]-5-(trifluoromethyl)-1H-benzimidazole, 5-Bromo-1-{[(3S)-1-(cyclopropylcarbonyl)-3-pyrrolidinyl]methyl}-6-fluoro-2-[4-(1H-indol-5-yl)phenyl]-1H-benzimidazole, 2-(4-Biphenylyl)-5-bromo-1-{[(3S)-1-(cyclopropylcarbonyl)-3-pyrrolidinyl]methyl}-6-fluoro-1H-benzimidazole, 5-Bromo-1-{[(3S)-1-(cyclopropylcarbonyl)-3-pyrrolidmyl]methyl}-6-fluoro-2-[4′-(methyloxy)-4-biphenylyl]-1H-benzimidazole, 1-{[(3S)-1-(cyclopropylcarbonyl)-3-pyrrolidinyl]methyl}-2-[4-(1H-indol-6-yl)phenyl]-6-(methyloxy)-1H-benzimidazole, 4′-[1-{[(3S)-1-(cyclopropylcarbonyl)-3-pyrrolidinyl]methyl}-6-(methyloxy)-1H-benzimidazol-2-yl]-3-biphenylol, 1-{[(3S)-1-(cyclopropylcarbonyl)-3-pyrrolidmyl]methyl}-2-(4′-fluoro-4-biphenylyl)-6-(methyloxy)-1H-benzimidazole, 1-{[(3S)-1-(cyclopropylcarbonyl)-3-pyrrolidmyl]methyl}-6-(methyloxy)-2-[4′-(methyloxy)-4-biphenylyl]-1H-benzimidazole, 1-{[(3S)-1-(cyclopropylcarbonyl)-3-pyrrolidinyl]methyl}-2-[4-(1H-indol-5-yl)phenyl]-6-(methyloxy)-1H-benzimidazole, 5-{4-[1-{[(3S)-1-(cyclopropylcarbonyl)-3-pyrrolidinyl]methyl}-6-(methyloxy)-1H-benzimidazol-2-yl]phenyl}-1H-indazole, 6-{4-[1-{[(3S)-1-(cyclopropylcarbonyl)-3-pyrrolidinyl]methyl}-6-(methyloxy)-1H-benzimidazol-2-yl]phenyl}-1H-indazole, 2-(4-Biphenylyl)-1-{[(3S)-1-(cyclopropylcarbonyl)-3-pyrrolidinyl]methyl}-6-(methyloxy)-1H-benzimidazole, 2-[4-(1-Benzofuran-5-yl)phenyl]-1-{[(3S)-1-(cyclopropylcarbonyl)-3-pyrrolidinyl]methy 1}-6-(methyloxy)-1H-benzimidazole, 4′-(1-{[(3S)-1-(cyclopropylcarbonyl)-3-pyrrolidinyl]methyl}-6-methyl-1H-benzimidazol-2-yl)-3-biphenylol, 1-{[(3S)-1-(cyclopropylcarbonyl)-3-pyrrolidinyl]methyl}-2-(4′-fluoro-4-biphenylyl)-6-methyl-1H-benzimidazole, 1-{[(3S)-1-(cyclopropylcarbonyl)-3-pyrrolidinyl]methyl}-6-methyl-2-[4′-(methyloxy)-4-biphenylyl]-1H-benzimidazole, 1-{[(3S)-1-(cyclopropylcarbonyl)-3-pyrrolidinyl]methyl}-2-[4-(1H-indol-5-yl)phenyl]-6-methyl-1H-benzimidazole, 1-{[(3S)-1-(cyclopropylcarbonyl)-3-pyrrolidinyl]methyl}-2-[4-(1H-indol-6-yl)phenyl]-6-methyl-1H-benzimidazole, 6-[4-(1-{[(3S)-1-(cyclopropylcarbonyl)-3-pyrrolidinyl]methyl}-6-methyl-1H-benzimidazol-2-yl)phenyl]-1H-indazole, 5-[4-(1-{[(3S)-1-(cyclopropylcarbonyl)-3-pyrrolidinyl]methyl}-6-methyl-1H-benzimidazol-2-yl)phenyl]-1H-indazole, 2-(4-Biphenylyl)-1-{[(3S)-1-(cyclopropylcarbonyl)-3-pyrrolidinyl]methyl}-6-methyl-1H-benzimidazole, 2-[4-(1-Benzofuran-5-yl)phenyl]-1-{[(3S)-1-(cyclopropylcarbonyl)-3-pyrrolidinyl]methy 1}-6-(methyl)-1H-benzimidazole, 4′-[1-{[(3S)-1-(cyclopropylcarbonyl)-3-pyrrolidinyl]methyl}-6-(trifluoromethyl)-1H-benzimidazol-2-yl]-3-biphenylol, 1-{[(3S)-1-(cyclopropylcarbonyl)-3-pyrrolidinyl]methyl}-2-(4′-fluoro-4-biphenylyl)-6-(trifluoromethyl)-1H-benzimidazole, 1-{[(3S)-1-(cyclopropylcarbonyl)-3-pyrrolidinyl]methyl}-2-[4′-(methyloxy)-4-biphenylyl]-6-(trifluoromethyl)-1H-benzimidazole, 1-{[(3S)-1-(cyclopropylcarbonyl)-3-pyrrolidinyl]methyl}-2-[4-(1H-indol-5-yl)phenyl]-6-(trifluoromethyl)-1H-benzimidazole, 1-{[(3S)-1-(cyclopropylcarbonyl)-3-pyrrolidinyl]methyl}-2-[4-(1H-indol-6-yl)phenyl]-6-(trifluoromethyl)-1H-benzimidazole, 5-{4-[1-{[(3S)-1-(cyclopropylcarbonyl)-3-pyrrolidinyl]methyl}-6-(trifluoromethyl)-1H-benzimidazol-2-yl]phenyl}-1H-indazole, 6-{4-[1-{[(3S)-1-(cyclopropylcarbonyl)-3-pyrrolidinyl]methyl}-6-(trifluoromethyl)-1H-benzimidazol-2-yl]phenyl}-1H-indazole, 2-(4-biphenylyl)-1-{[(3S)-1-(cyclopropylcarbonyl)-3-pyrrolidinyl]methyl}-6-(trifluoromethyl)-1H-benzimidazole, 2-[4-(1-benzofuran-5-yl)phenyl]-1-{[(3S)-1-(cyclopropylcarbonyl)-3-pyrrolidinyl]methyl}-6-(trifluoromethyl)-1H-benzimidazole, 2-(4-Biphenylyl)-6-bromo-1-{[(3S)-1-(cyclopropylcarbonyl)-3-pyrrolidinyl]methyl}-1H-benzimidazole, 4′-(6-bromo-1-{[(3S)-1-(cyclopropylcarbonyl)-3-pyrrolidinyl]methyl}-1H-benzimidazol-2-yl)-3-biphenylol, 6-Bromo-1-{[(3S)-1-(cyclopropylcarbonyl)-3-pyrrolidmyl]methyl}-2-(4′-fluoro-4-biphenylyl)-1H-benzimidazole, 6-Bromo-1-{[(3S)-1-(cyclopropylcarbonyl)-3-pyrrolidinyl]methyl}-2-[4′-(methyloxy)-4-biphenylyl]-1H-benzimidazole, 6-Bromo-1-{[(3S)-1-(cyclopropylcarbonyl)-3-pyrrolidmyl]methyl}-2-[4-(1H-indol-5-yl)phenyl]-1H-benzimidazole, 6-Bromo-1-{[(3S)-1-(cyclopropylcarbonyl)-3-pyrrolidmyl]methyl}-2-[4-(1H-indol-6-yl)phenyl]-1H-benzimidazole, 5-[4-(6-Bromo-1-{[(3S)-1-(cyclopropylcarbonyl)-3-pyrrolidinyl]methyl}-1H-benzimidazol-2-yl)phenyl]-1H-indazole, 6-[4-(B-bromo-1-{[(3S)-1-(cyclopropylcarbonyl)-3-pyrrolidmyl]methyl}-1H-benzimidazol-2-yl)phenyl]-1H-indazole, 2-[4-(1-Benzofuran-5-yl)phenyl]-6-bromo-1-{[(3S)-1-(cyclopropylcarbonyl)-3-pyrrolidinyl]methyl}-1H-benzimidazole, 2-(4-Biphenylyl)-1-{[(3S)-1-(cyclopropylcarbonyl)-3-pyrrolidmyl]methyl}-N-methyl-1H-benzimidazole-6-carboxamide, 1-{[(3S)-1-(cyclopropylcarbonyl)-3-pyrrolidmyl]methyl}-2-(4′-fluoro-4-biphenylyl)-N-methyl-1H-benzimidazole-6-carboxamide, 1-{[(3S)-1-(cyclopropylcarbonyl)-3-pyrrolidmyl]methyl}-N-methyl-2-[4′-(methyloxy)-4-biphenylyl]-1H-benzimidazole-6-carboxamide, 1-{[(3S)-1-(cyclopropylcarbonyl)-3-pyrrol idinyl]methyl}-2-(4′-fluoro-4-biphenylyl)-6-[(4-methyl-1-piperazinyl)carbonyl]-1H-benzimidazole, 1-{[(3S)-1-(cyclopropylcarbonyl)-3-pyrrolidinyl]methyl}-2-[4-(1H-indol-6-yl)phenyl]-6-[(4-methyl-1-piperazinyl)carbonyl]-1H-benzimidazole, 1-{[(3S)-1-(cyclopropylcarbonyl)-3-pyrrolidinyl]methyl}-2-[4-(1H-indol-5-yl)phenyl]-6-[(4-methyl-1-piperazinyl)carbonyl]-1H-benzimidazole, 1-{[(3S)-1-(cyclopropylcarbonyl)-3-pyrrolidinyl]methyl}-7-methyl-2-[4′-(methyloxy)-4-biphenylyl]-1H-benzimidazole, 1-{[(3S)-1-(cyclopropylcarbonyl)-3-pyrrolidinyl]methyl}-2-[4-(1H-indol-5-yl)phenyl]-7-methyl-1H-benzimidazole, 1-{[(3S)-1-(cyclopropylcarbonyl)-3-pyrrolidinyl]methyl}-2-[4-(1H-indol-6-yl)phenyl]-7-methyl-1H-benzimidazole, 5-[4-(1-{[(3S)-1-(cyclopropylcarbonyl)-3-pyrrolidinyl]methyl}-7-methyl-1H-benzimidazol-2-yl)phenyl]-1H-indazole, 2-(4-Biphenylyl)-1-{[(3S)-1-(cyclopropylcarbonyl)-3-pyrrolidinyl]methyl}-7-methyl-1H-benzimidazole, 4′-(1-{[(3S)-1-(cyclopropylcarbonyl)-3-pyrrolidinyl]methyl}-7-methyl-1H-benzimidazol-2-yl)-3-biphenylol, 1-{[(3S)-1-(cyclopropylcarbonyl)-3-pyrrolidinyl]methyl}-2-(4′-fluoro-4-biphenylyl)-7-methyl-1H-benzimidazole, 6-[4-(1-{[(3S)-1-(cyclopropylcarbonyl)-3-pyrrolidinyl]methyl}-7-methyl-1H-benzimidazol-2-yl)phenyl]-1H-indazole, 2-[4-(1-Benzofuran-5-yl)phenyl]-1-{[(3S)-1-(cyclopropylcarbonyl)-3-pyrrolidinyl]methyl}-7-methyl-1H-benzimidazole, 1-{[(3S)-1-(cyclopropylcarbonyl)-3-pyrrolidinyl]methyl}-2-(4′-fluoro-4-biphenylyl)-7-(trifluoromethyl)-1H-benzimidazole, 1-{[(3S)-1-(Cyclopropylcarbonyl)-3-pyrrolidinyl]methyl}-2-[4′-(methyloxy)-4-biphenylyl]-7-(trifluoromethyl)-1H-benzimidazole, 2-[4-(1-Benzofuran-5-yl)phenyl]-1-{[(3S)-1-(cyclopropylcarbonyl)-3-pyrrolidinyl]methyl}-7-(trifluoromethyl)-1H-benzimidazole, 1-{[(3S)-1-(cyclopropylcarbonyl)-3-pyrrolidinyl]methyl}-2-[4-(1H-indol-5-yl)phenyl]-7-(trifluoromethyl)-1H-benzimidazole, 5-{4-[1-{[(3S)-1-(cyclopropylcarbonyl)-3-pyrrolidinyl]methyl}-7-(trifluoromethyl)-1H-benzimidazol-2-yl]phenyl}-1H-indazole, 6-{4-[1-{[(3S)-1-(cyclopropylcarbonyl)-3-pyrrolidinyl]methyl}-7-(trifluoromethyl)-1H-benzimidazol-2-yl]phenyl}-1H-indazole, 1-{[(3S)-1-(cyclopropylcarbonyl)-3-pyrrolidinyl]methyl}-2-[4-(1H-indol-6-yl)phenyl]-7-(trifluoromethyl)-1H-benzimidazole, 2-(4-Biphenylyl)-7-bromo-1-{[(3S)-1-(cyclopropylcarbonyl)-3-pyrrolidinyl]methyl}-1H-benzimidazole, 4′-(7-Bromo-1-{[(3S)-1-(cyclopropylcarbonyl)-3-pyrrolidinyl]methyl}-1H-benzimidazol-2-yl)-3-biphenylol, 7-Bromo-1-{[(3S)-1-(cyclopropylcarbonyl)-3-pyrrolidinyl]methyl}-2-(4′-fluoro-4-biphenylyl)-1H-benzimidazole, 7-Bromo-1-{[(3S)-1-(cyclopropylcarbonyl)-3-pyrrolidinyl]methyl}-2-[4′-(methyloxy)-4-biphenylyl]-1H-benzimidazole, 7-Bromo-1-{[(3S)-1-(cyclopropylcarbonyl)-3-pyrrolidinyl]methyl}-2-[4-(1H-indol-5-yl)phenyl]-1H-benzimidazole, 5-[4-(7-Bromo-1-{[(3S)-1-(cyclopropylcarbonyl)-3-pyrrolidinyl]methyl}-1H-benzimidazol-2-yl)phenyl]-1H-indazole, 7-Bromo-1-{[(3S)-1-(cyclopropylcarbonyl)-3-pyrrolidmyl]methyl}-2-[4-(1H-indol-6-yl)phenyl]-1H-benzimidazole, 6-[4-(7-Bromo-1-{[(3S)-1-(cyclopropylcarbonyl)-3-pyrrolidinyl]methyl}-1H-benzimidazol-2-yl)phenyl]-1H-indazole, 2-[4-(1-Benzofuran-5-yl)phenyl]-7-bromo-1-{[(3S)-1-(cyclopropylcarbonyl)-3-pyrrolidinyl]methyl}-1H-benzimidazole, 1-{[(3S)-1-(cyclopropylcarbonyl)-3-pyrrolidmyl]methyl}-2-(3′-hydroxy-4-biphenylyl)-N-methyl-1H-benzimidazole-7-carboxamide, 1-{[(3S)-1-(cyclopropylcarbonyl)-3-pyrrolidmyl]methyl}-2-(4′-fluoro-4-biphenylyl)-N-methyl-1H-benzimidazole-7-carboxamide, 1-{[(3S)-1-(cyclopropylcarbonyl)-3-pyrrolidinyl]methyl}-2-[4-(1H-indol-5-yl)phenyl]-N-methyl-1H-benzimidazole-7-carboxamide, 1-{[(3S)-1-(cyclopropylcarbonyl)-3-pyrrolidinyl]methyl}-2-[4-(1H-indol-6-yl)phenyl]-N-methyl-1H-benzimidazole-7-carboxamide, 1-{[(3S)-1-(cyclopropylcarbonyl)-3-pyrrolidinyl]methyl}-2-[4-(1H-indazol-5-yl)phenyl]-N-methyl-1H-benzimidazole-7-carboxamide, 1-{[(3S)-1-(cyclopropylcarbonyl)-3-pyrrolidinyl]methyl}-2-[4-(1H-indazol-6-yl)phenyl]-N-methyl-1H-benzimidazole-7-carboxamide, 2-(4-Diphenylyl)-1-{[(3S)-1-(cyclopropylcarbonyl)-3-pyrrolidinyl]methyl}-N-methyl-1H-benzimidazole-7-carboxamide, 1-{[(3S)-1-(cyclopropylcarbonyl)-3-pyrrolidmyl]methyl}-N-methyl-2-[4′-(methyloxy)-4-biphenylyl]-1H-benzimidazole-7-carboxamide, 2-[4-(1-Benzofuran-5-yl)phenyl]-1-{[(3S)-1-(cyclopropylcarbonyl)-3-pyrrolidinyl]methyl}-N-methyl-1H-benzimidazole-7-carboxamide, 2-(4-Biphenylyl)-1-({(3S)-1-[(dimethy methyl-1H-benzimidazole-6-carboxamide, 2-(4-Biphenylyl)-N-methyl-1-({(3RS)-1-[(3-methyl-5-isoxazolyl)carbonyl]-3-pyrrolidinyl}methyl)-1H-benzimidazole-6-carboxamide, 1-{[(3S)-1-(cyclopropylcarbonyl)-3-pyrrolidinyl]methyl}-2-[4-(1H-pyrrolo[3,2-b]pyridin-6-yl)phenyl]-5-(trifluoromethyl)-1H-benzimidazole, 1-{[(3S)-1-(cyclopropylcarbonyl)-3-pyrrolidinyl]methyl}-2-(4-imidazo[1,2-a]pyridin-7-ylphenyl)-5-(trifluoromethyl)-1H-benzimidazole, 5-{4-[1-{[(3S)-1-(cyclopropylcarbonyl)-3-pyrrolidinyl]methyl}-5-(trifluoromethyl)-1H-benzimidazol-2-yl]phenyl}-1H-pyrazolo[3,4-b]pyridine, 5-{4-[1-{[(3S)-1-(cyclopropylcarbonyl)-3-pyrrolidinyl]methyl}-5-(trifluoromethyl)-1H-benzimidazol-2-yl]phenyl}-1,3-benzoxazole, 6-{4-[1-{[(3S)-1-(cyclopropylcarbonyl)-3-pyrrolidinyl]methyl}-5-(trifluoromethyl)-1H-benzimidazol-2-yl]phenyl}-1,3-benzothiazole, 1-{[(3S)-1-(cyclopropylcarbonyl)-3-pyrrolidinyl]methyl}-2-[4-(1H-pyrrolo[2,3-b]pyridin-5-yl)phenyl]-5-(trifluoromethyl)-1H-benzimidazole, 1-{[(3S)-1-(cyclopropylcarbonyl)-3-pyrrolidinyl]methyl}-2-(4-imidazo[1,5-a]pyridin-5-ylphenyl)-5-(trifluoromethyl)-1H-benzimidazole, 1-{[(3S)-1-(cyclopropylcarbonyl)-3-pyrrolidinyl]methyl}-2-(4-imidazo[1,2-a]pyridin-5-ylphenyl)-5-(trifluoromethyl)-1H-benzimidazole, 2-[4-(1-Benzofuran-6-yl)phenyl]-1-{[(3S)-1-(cyclopropylcarbonyl)-3-pyrrolidinyl]methyl}-5-(trifluoromethyl)-1H-benzimidazole, 1-{[(3S)-1-(cyclopropylcarbonyl)-3-pyrrolidinyl]methyl}-2-(4-imidazo[1,2-a]pyridin-3-ylphenyl)-5-(trifluoromethyl)-1H-benzimidazole, 1-{[(3S)-1-(cyclopropylcarbonyl)-3-pyrrolidinyl]methyl}-2-[3′-(methylsulfonyl)-4-biphenylyl]-5-(trifluoromethyl)-1H-benzimidazole, 1-{[(3S)-1-(cyclopropylcarbonyl)-3-pyrrolidinyl]methyl}-2-[4′-(methylsulfonyl)-4-biphenylyl]-5-(trifluoromethyl)-1H-benzimidazole, 6-{4-[1-{[(3S)-1-(cyclopropylcarbonyl)-3-pyrrolidinyl]methyl}-5-(trifluoromethyl)-1H-benzimidazol-2-yl]phenyl}-1,3-benzoxazole, 5-{4-[1-{[(3S)-1-(cyclopropylcarbonyl)-3-pyrrolidinyl]methyl}-5-(trifluoromethyl)-1H-benzimidazol-2-yl]phenyl}-1,3-dihydro-2H-indol-2-one, 1-{[(3S)-1-(cyclopropylcarbonyl)-3-pyrrolidinyl]methyl}-2-[4-(2,3-dihydro-1H-indol-5-yl)phenyl]-5-(trifluoromethyl)-1H-benzimidazole, 1-{[(3S)-1-(cyclopropylcarbonyl)-3-pyrrolidinyl]methyl}-2-[4-(1H-pyrrolo[2,3-b]pyridin-6-yl)phenyl]-5-(trifluoromethyl)-1H-benzimidazole, 6-{4-[1-{[(3S)-1-(cyclopropylcarbonyl)-3-pyrrolidinyl]methyl}-5-(trifluoromethyl)-1H-benzimidazol-2-yl]phenyl}-1H-pyrazolo[3,4-b]pyridine, 2-[4-(1-Benzofuran-3-yl)phenyl]-1-{[(3S)-1-(cyclopropylcarbonyl)-3-pyrrolidinyl]methyl}-5-(trifluoromethyl)-1H-benzimidazole, 4′-[1-{[(3S)-1-(cyclopropylcarbonyl)-3-pyrrolidinyl]methyl}-5-(trifluoromethyl)-1H-benzimidazol-2-yl]-4-biphenylcarbonitrile, 6-{4-[1-{[(3S)-1-(cyclopropylcarbonyl)-3-pyrrolidinyl]methyl}-5-(trifluoromethyl)-1H-benzimidazol-2-yl]phenyl}quinazoline, 1-{[(3S)-1-(cyclopropylcarbonyl)-3-pyrrolidinyl]methyl}-2-[4-(1H-pyrrolo[3,2-c]pyridin-3-yl)phenyl]-5-(trifluoromethyl)-1H-benzimidazole, 2-[4-(1H-Benzimidazol-5-yl)phenyl]-1-{[(3S)-1-(cyclopropylcarbonyl)-3-pyrrolidinyl]methyl}-5-(trifluoromethyl)-1H-benzimidazole, 6-{4-[1-{[(3S)-1-(cyclopropylcarbonyl)-3-pyrrolidinyl]methyl}-5-(trifluoromethyl)-1H-benzimidazol-2-yl]phenyl}-1 (2H)-isoquinolinone, 1-{[(3S)-1-(cyclopropylcarbonyl)-3-pyrrolidinyl]methyl}-2-[4-(2-methyl-1H-indol-5-yl)phenyl]-5-(trifluoromethyl)-1H-benzimidazole, 2-[4-(1-Benzofuran-5-yl)phenyl]-1-{[(3S)-1-(cyclopropylcarbonyl)-3-pyrrolidinyl]methyl}-1H-benzimidazole-5-carbonitrile, 1-{[(3S)-1-(cyclopropylcarbonyl)-3-pyrrolidinyl]methyl}-2-[4-(1H-indol-5-yl)phenyl]-1H-benzimidazole-5-carbonitrile, 2-[4-(1-Benzofuran-5-yl)phenyl]-1-{[(3S)-1-(cyclopropylcarbonyl)-3-pyrrolidinyl]methyl}-1H-benzimidazole-6-carbonitrile, 1-{[(3S)-1-(cyclopropylcarbonyl)-3-pyrrolidinyl]methyl}-2-[4-(1H-indol-5-yl)phenyl]-1H-benzimidazole-6-carbonitrile, 1-{[(3S)-1-(cyclopropylcarbonyl)-3-pyrrolidinyl]methyl}-2-[4-(1H-indol-6-yl)phenyl]-1H-benzimidazole-6-carbonitrile, 2-[4-(1-Benzofuran-5-yl)phenyl]-1-{[(3S)-1-(cyclopropylcarbonyl)-3-pyrrolidinyl]methyl}-1H-benzimidazole-7-carbonitrile, 1-{[(3S)-1-(cyclopropylcarbonyl)-3-pyrrolidinyl]methyl}-2-[4-(1H-indol-6-yl)phenyl]-1H-benzimidazole-7-carbonitrile, 1-{[(3S)-1-(cyclopropylcarbonyl)-3-pyrrolidinyl]methyl}-2-[4-(1H-indol-5-yl)phenyl]-1H-benzimidazole-7-carbonitrile, N-[4′-(7-cyano-1-{[(3S)-1-(cyclopropylcarbonyl)-3-pyrrolidinyl]methyl}-1H-benzimidazol-2-yl)-3-biphenylyl]-N,N-dimethylsulfamide, 2-[4-(1-Benzofuran-5-yl)phenyl]-1-{[(3S)-1-(cyclopropylcarbonyl)-3-pyrrolidinyl]methyl}-1H-benzimidazole-4-carbonitrile, 1-{[(3S)-1-(cyclopropylcarbonyl)-3-pyrrolidinyl]methyl}-2-[4-(1H-indol-5-yl)phenyl]-1H-benzimidazole-4-carbonitrile, 1-{[(3S)-1-(cyclopropylcarbonyl)-3-pyrrolidinyl]methyl}-2-[4-(1H-indol-6-yl)phenyl]-1H-benzimidazole-4-carbonitrile, 5-[4-(1-{[(3S)-1-(cyclopropylcarbonyl)-3-pyrrolidinyl]methyl}-6-fluoro-1H-benzimidazol-2-yl)phenyl]-1H-indazole, 1-{[(3S)-1-(cyclopropylcarbonyl)-3-pyrrolidinyl]methyl}-6-fluoro-2-[4-(1H-indol-6-yl)phenyl]-1H-benzimidazole, 6-[4-(1-{[(3S)-1-(cyclopropylcarbonyl)-3-pyrrolidinyl]methyl}-6-fluoro-1H-benzimidazol-2-yl)phenyl]-1H-indazole, N-[4′-(1-{[(3S)-1-(cyclopropylcarbonyl)-3-pyrrolidinyl]methyl}-6-fluoro-1H-benzimidazol-2-yl)-3-biphenylyl]-N,N-dimethylsulfamide, 1-{[(3S)-1-(cyclopropylcarbonyl)-3-pyrrolidinyl]methyl}-6-fluoro-2-(4′-fluoro-4-biphenylyl)-1H-benzimidazole, 1-{[(3S)-1-(cyclopropylcarbonyl)-3-pyrrolidinyl]methyl}-6-fluoro-2-[4-(1H-indol-5-yl)phenyl]-1H-benzimidazole, 2-[4-(1-Benzofuran-5-yl)phenyl]-1-{[(3S)-1-(cyclopropylcarbonyl)-3-pyrrolidinyl]methyl}-6-fluoro-1H-benzimidazole, 2-[4-(1-Benzofuran-5-yl)phenyl]-1-{[(3S)-1-(cyclopropylcarbonyl)-3-pyrrolidinyl]methyl}-5-fluoro-1H-benzimidazole, 1-{[(3S)-1-(cyclopropylcarbonyl)-3-pyrrolidinyl]methyl}-5-fluoro-2-[4-(1H-indol-6-yl)phenyl]-1H-benzimidazole, 1-{[(3S)-1-(cyclopropylcarbonyl)-3-pyrrolidinyl]methyl}-5-fluoro-2-[4-(1H-indol-5-yl)phenyl]-1H-benzimidazole, and 5-[4-(1-{[(3S)-1-(cyclopropylcarbonyl)-3-pyrrolidinyl]methyl}-5-fluoro-1H-benzimidazol-yl)phenyl]-1H-indazole.

In some embodiments, the FASN inhibitor is one of the following:

| | Compound |
|---|---|
| 726 | 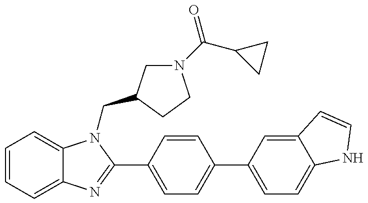 |
| 727 | 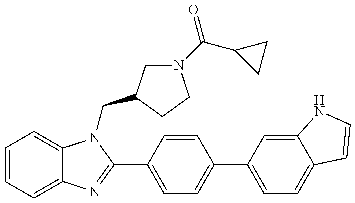 |
| 728 | 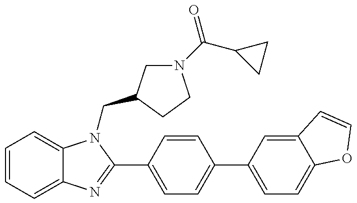 |
| 729 | 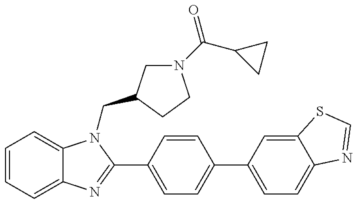 |
| 730 | 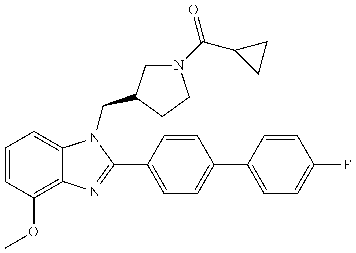 |
| 731 | 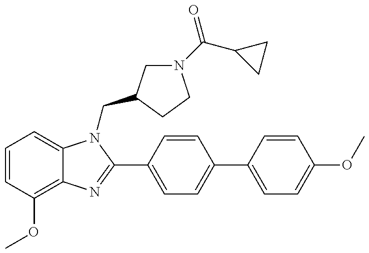 |
| 732 | 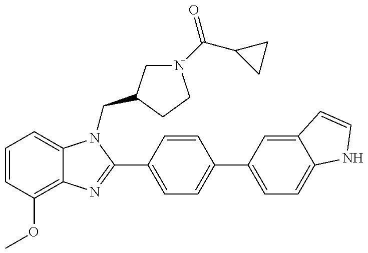 |
| 733 | 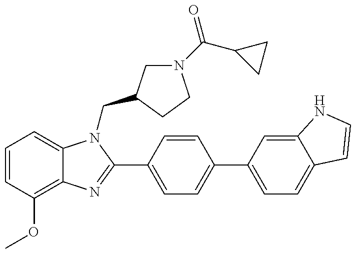 |
| 734 | 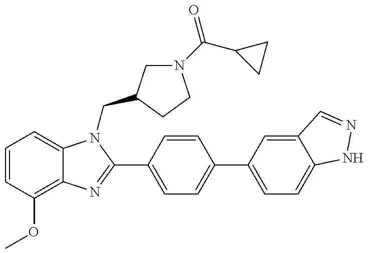 |
| 735 | 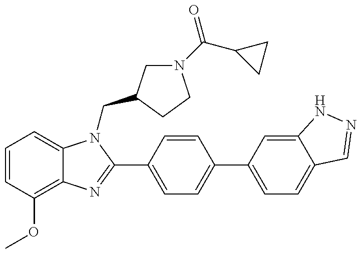 |
| 736 | 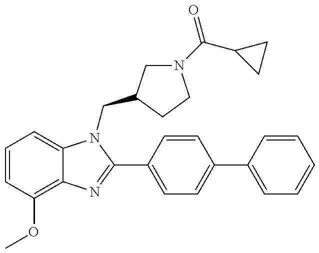 |
| 737 | 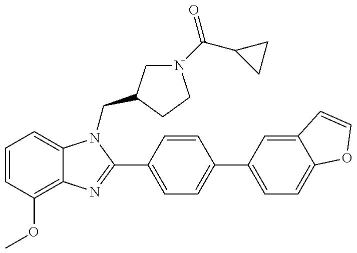 |
| 738 | 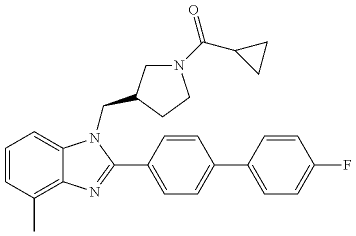 |
| 739 | 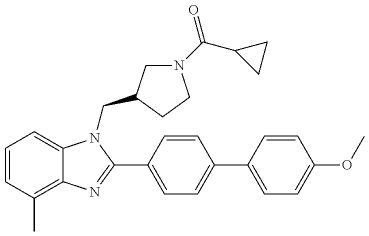 |
| 740 | 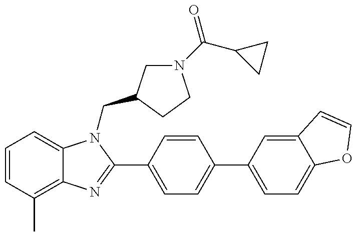 |
| 741 | 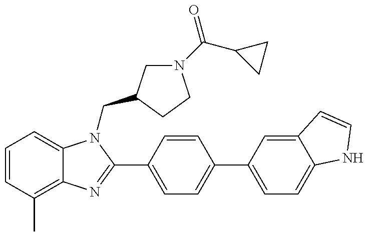 |
| 742 | 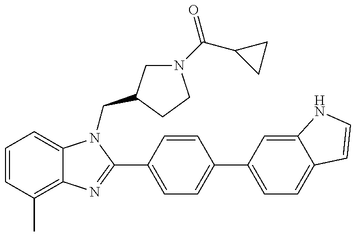 |
| 743 | 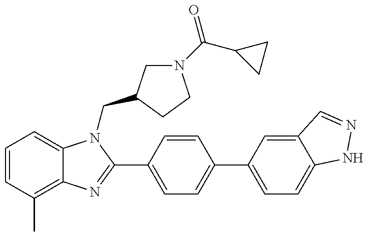 |
| 744 | 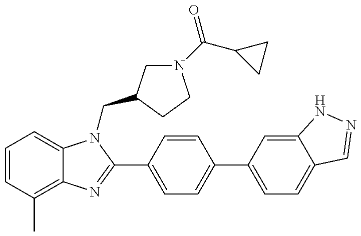 |
| 745 | 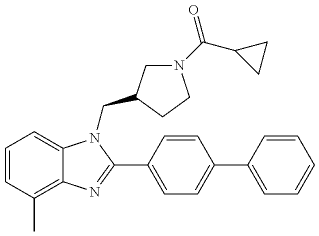 |
| 746 | 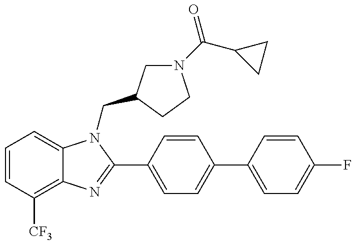 |
| 747 | 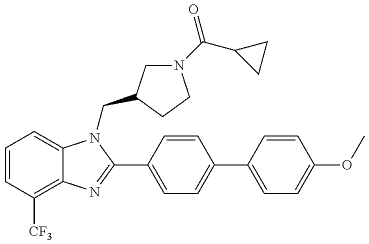 |
| 748 | 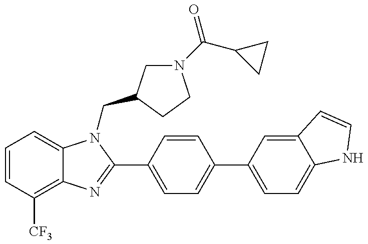 |
| 749 | 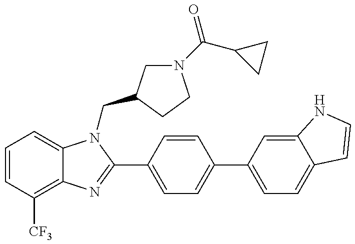 |
| 750 | 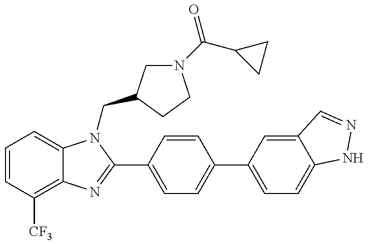 |
| 751 | 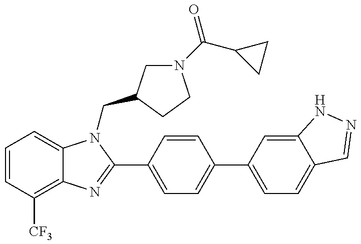 |
| 752 | 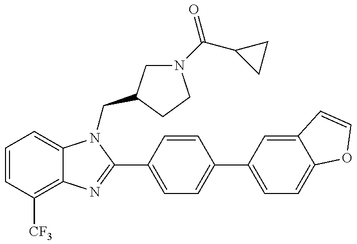 |
| 753 | 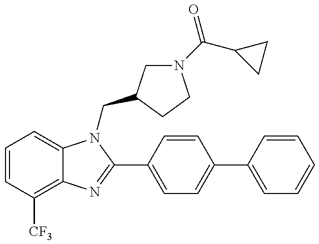 |
| 754 | 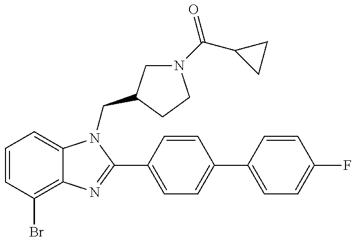 |
| 755 | 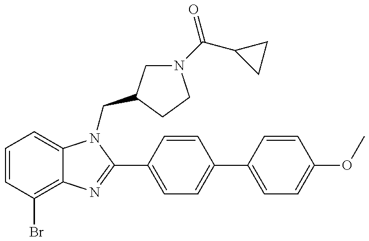 |
| 756 | 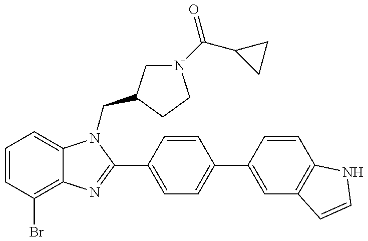 |
| 757 | 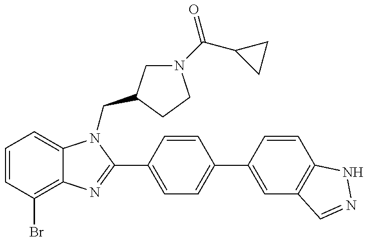 |
| 758 | 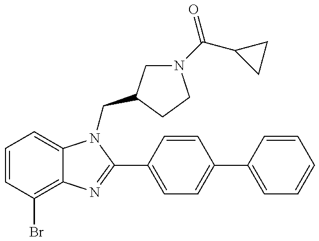 |
| 759 | 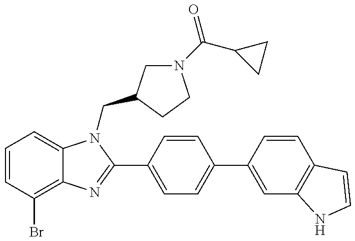 |
| 760 | 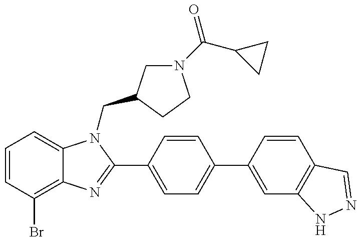 |
| 761 | 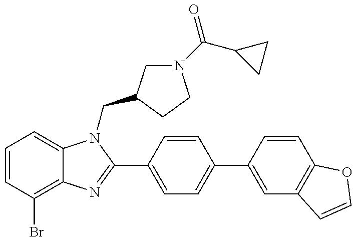 |
| 762 | 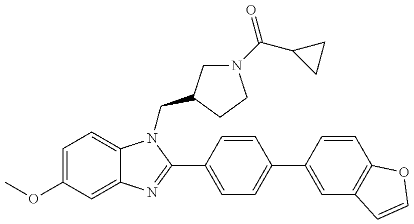 |
| 763 | 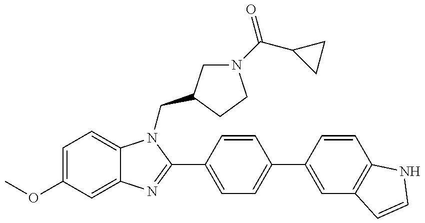 |
| 764 | 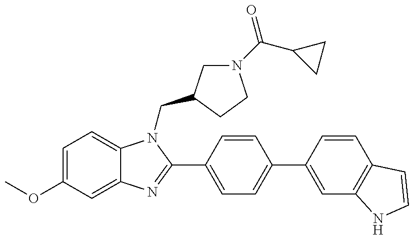 |
| 765 | 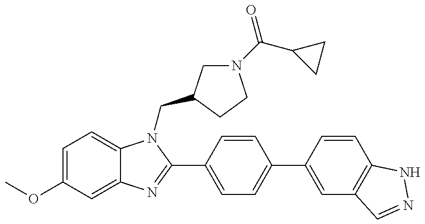 |
| 766 | 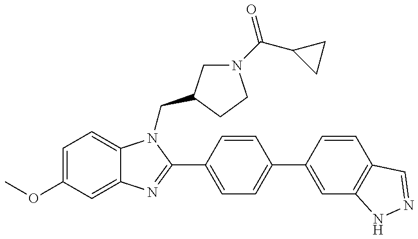 |
| 767 | 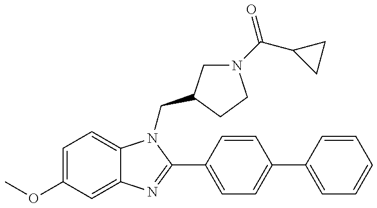 |
| 768 | 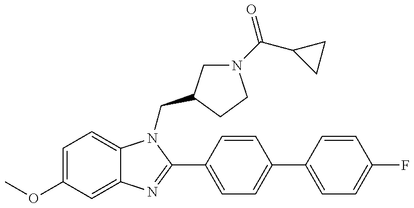 |
| 769 | 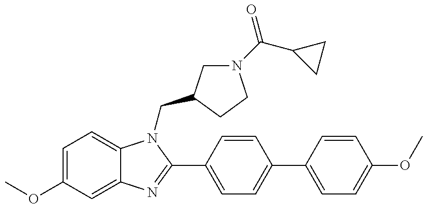 |
| 770 | 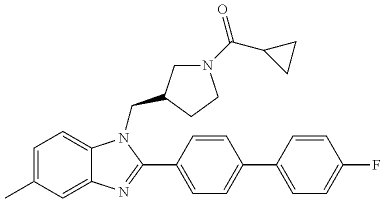 |
| 771 | 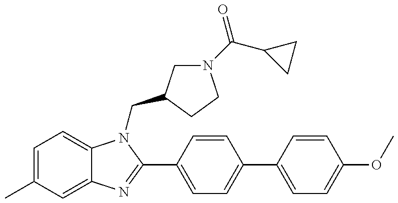 |
| 772 | 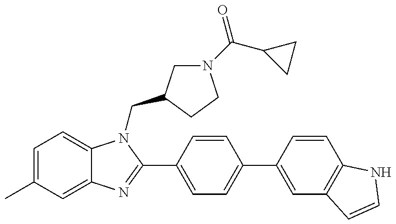 |
| 773 | 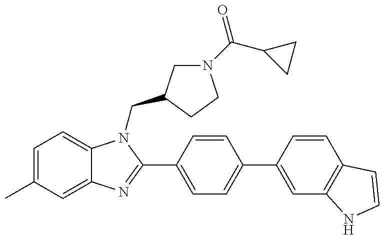 |
| 774 | 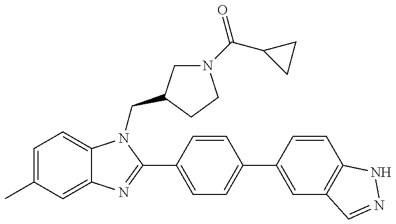 |
| 775 | 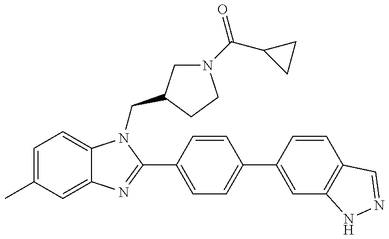 |
| 776 | 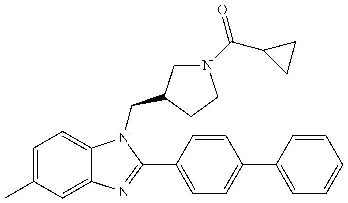 |
| 777 | 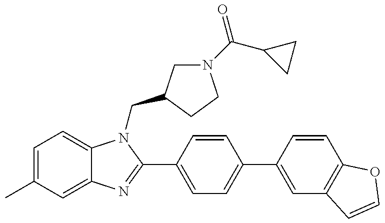 |
| 778 | 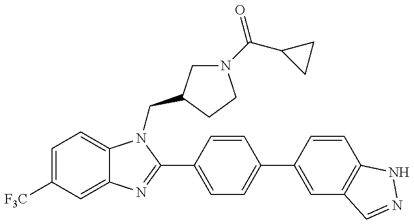 |
| 779 | 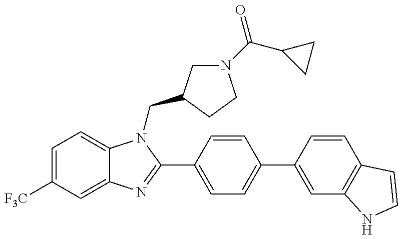 |
| 780 | 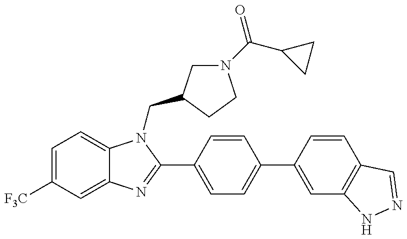 |
| 781 | 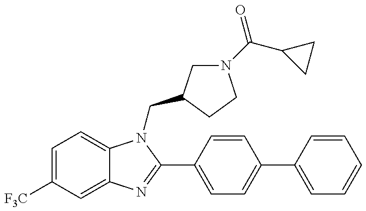 |
| 782 | 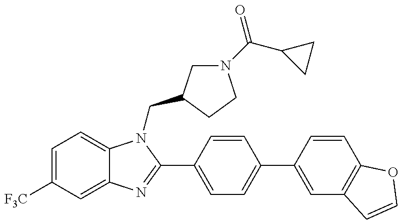 |
| 783 | 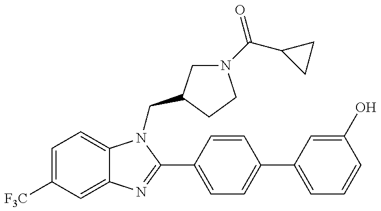 |
| 784 | 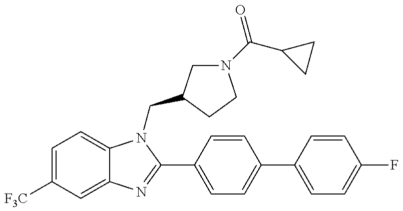 |
| 785 | 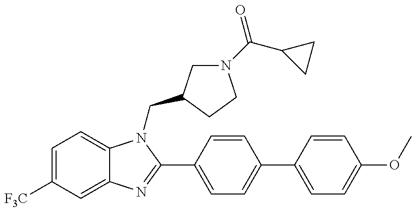 |
| 786 | 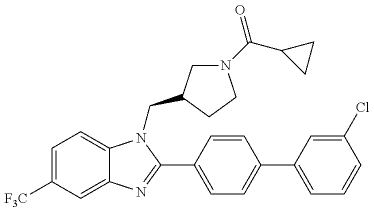 |
| 787 | 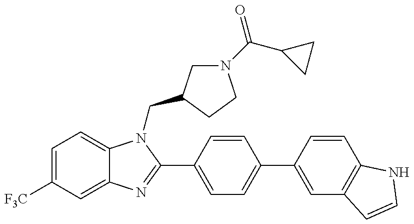 |
| 788 | 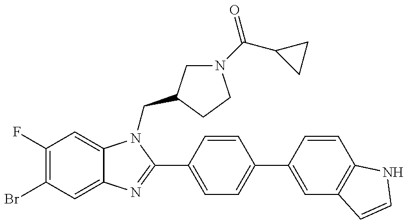 |
| 789 | 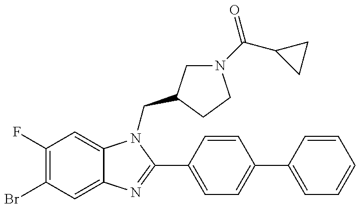 |
| 790 | 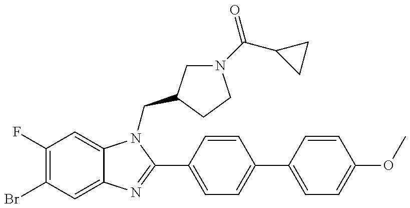 |
| 791 | 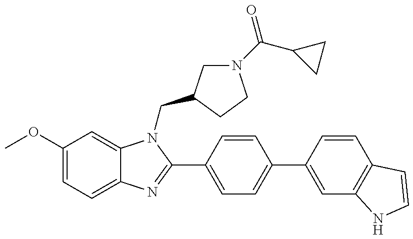 |
| 792 | 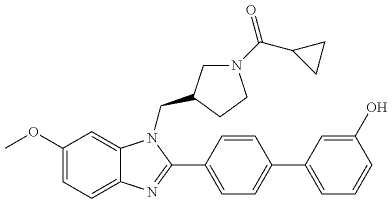 |
| 793 | 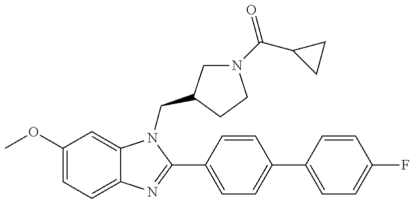 |
| 794 | 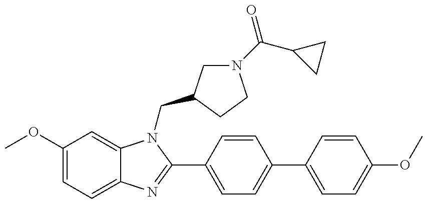 |
| 795 | 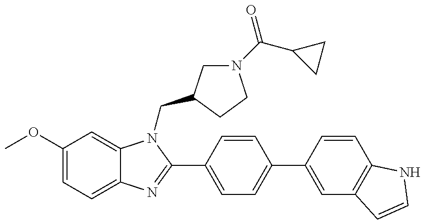 |
| 796 | 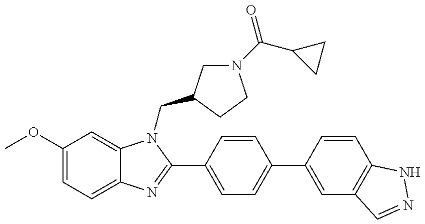 |
| 797 | 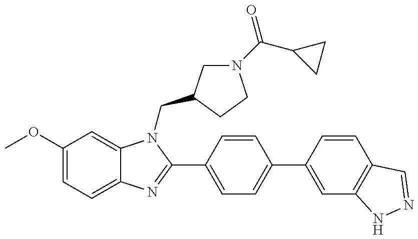 |
| 798 | 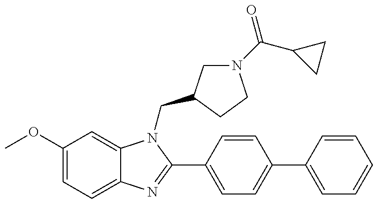 |
| 799 | 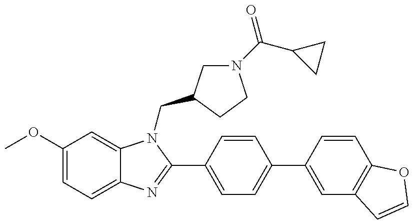 |
| 800 | 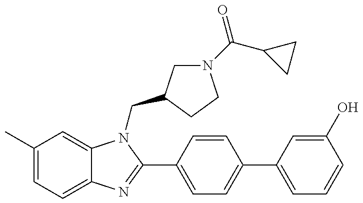 |
| 801 | 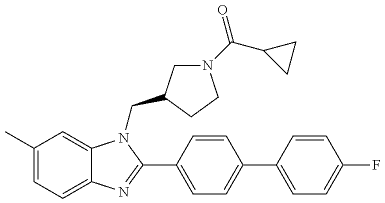 |
| 802 | 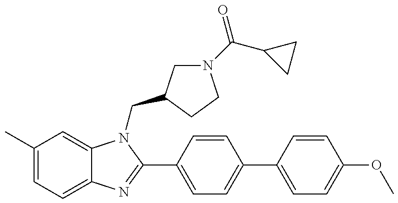 |
| 803 | 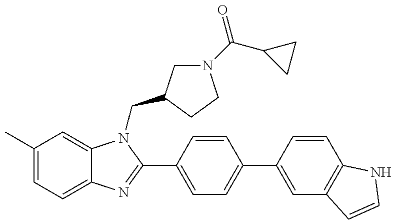 |
| 804 | 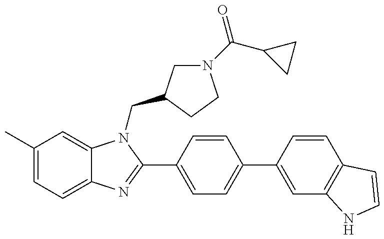 |
| 805 | 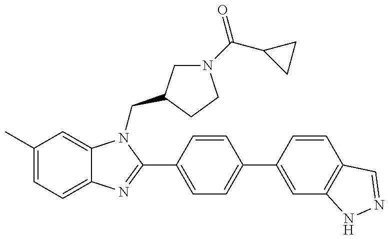 |
| 806 | 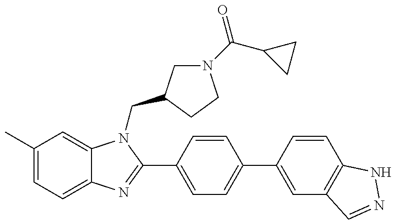 |
| 807 | 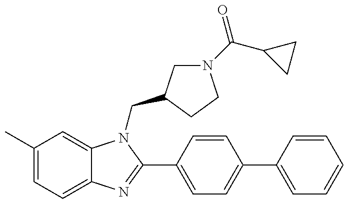 |
| 808 | 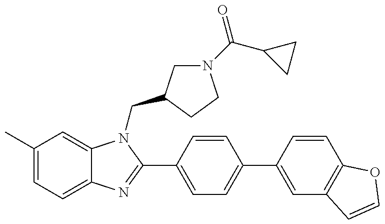 |
| 809 | 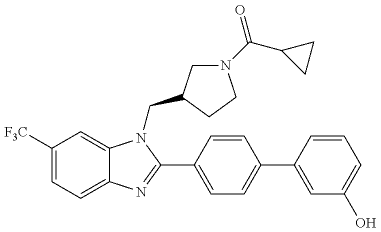 |
| 810 | 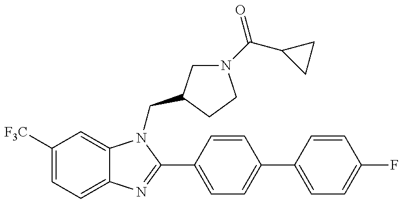 |
| 811 | 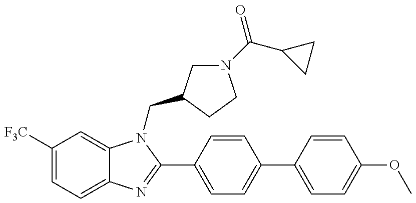 |
| 812 | 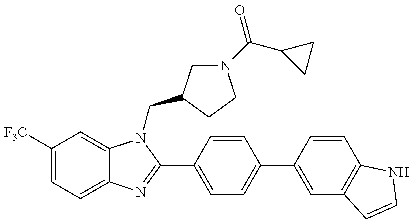 |
| 813 | 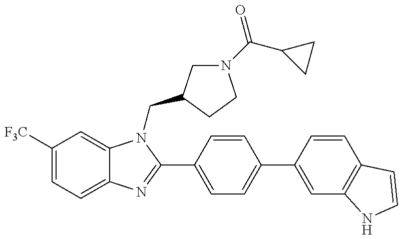 |
| 814 | 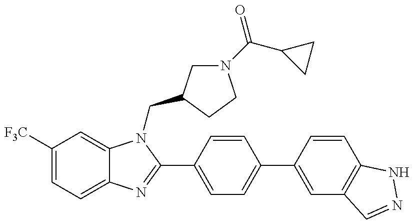 |
| 815 | 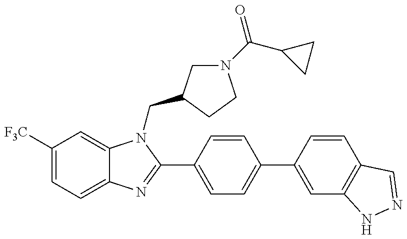 |
| 816 | 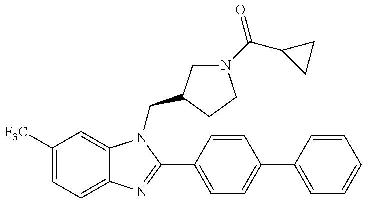 |
| 817 | 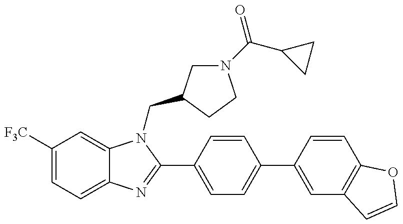 |
| 818 | 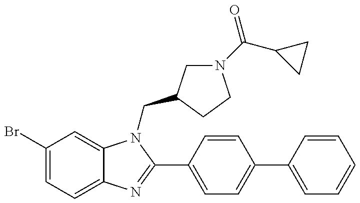 |
| 819 | 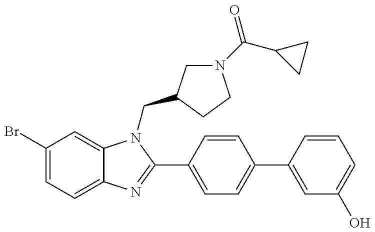 |
| 820 | 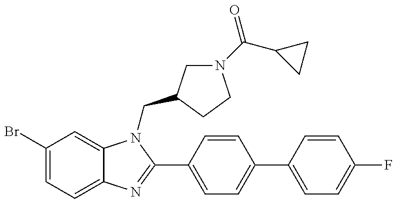 |
| 821 | 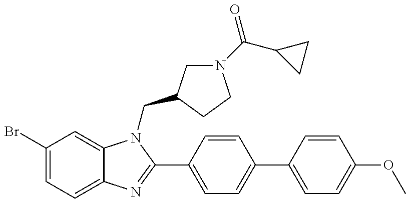 |
| 822 | 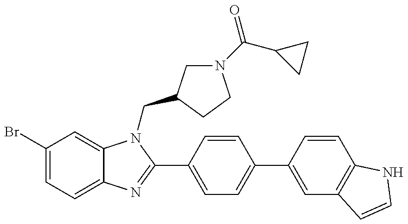 |
| 823 | 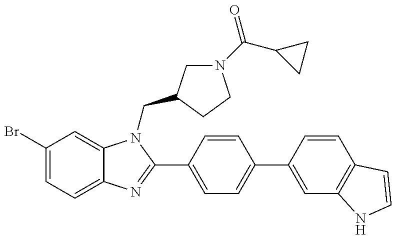 |
| 824 | 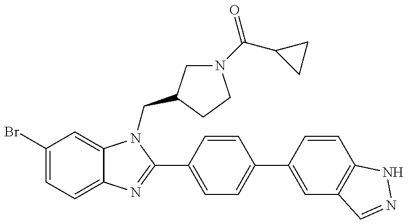 |
| 825 | 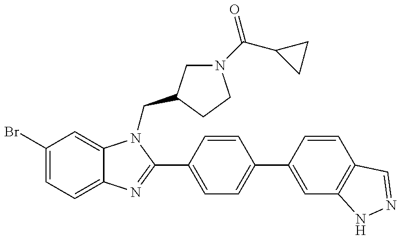 |
| 826 | 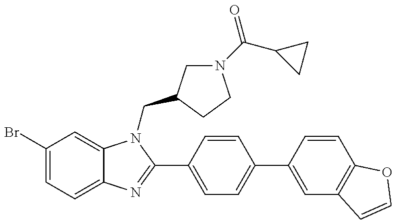 |
| 827 | 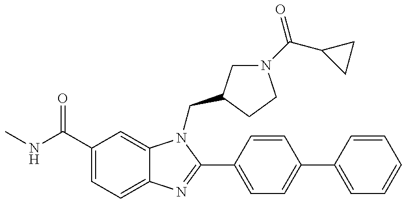 |
| 828 | 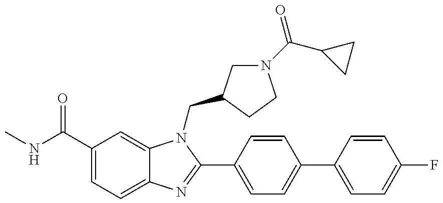 |
| 829 | 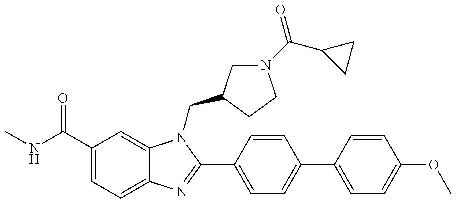 |
| 830 | 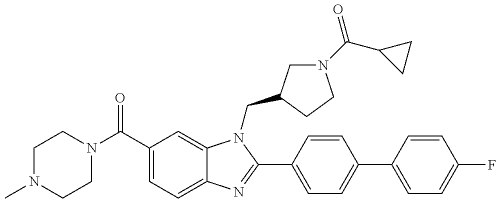 |
| 831 | 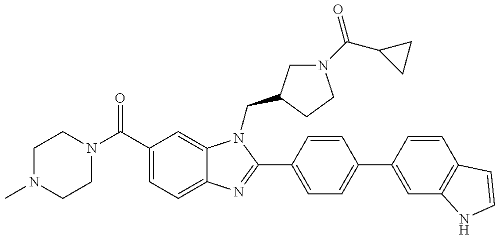 |
| 832 | 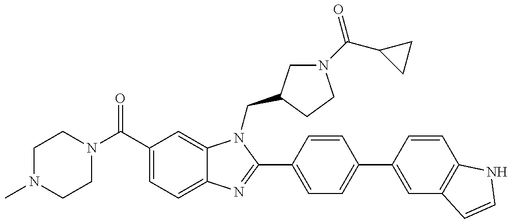 |
| 833 | 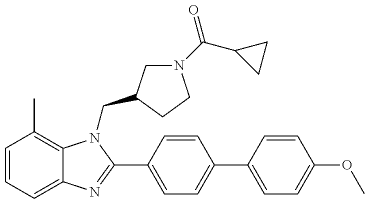 |
| 834 | 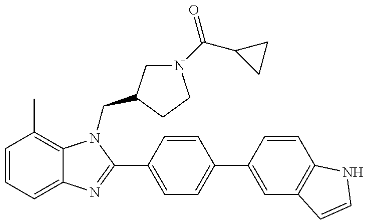 |
| 835 | 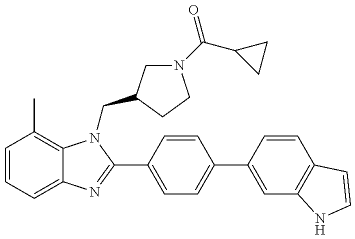 |
| 836 | 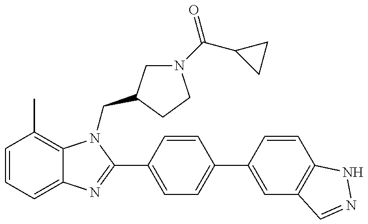 |
| 837 | 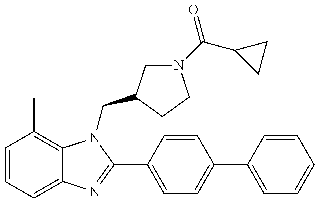 |
| 838 | 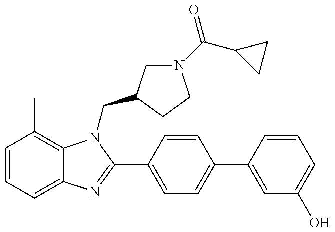 |
| 839 | 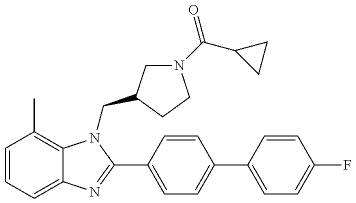 |
| 840 | 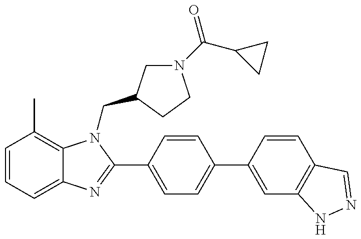 |
| 841 | 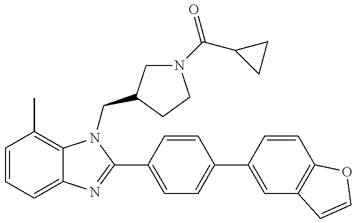 |
| 842 | 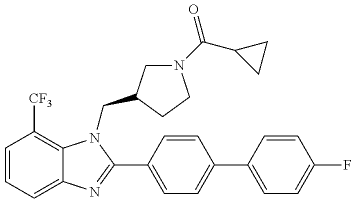 |
| 843 | 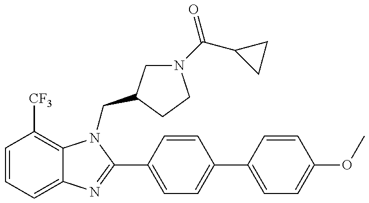 |
| 844 | 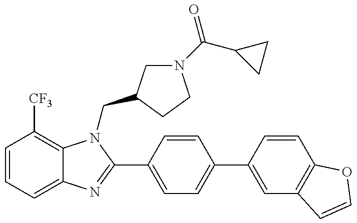 |
| 845 | 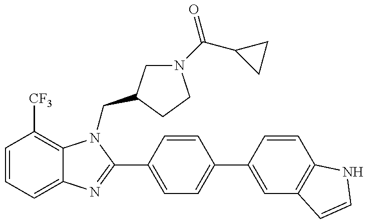 |
| 846 | 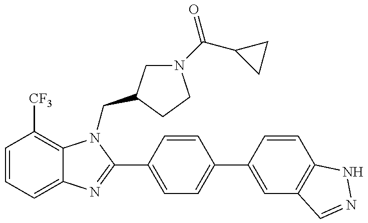 |
| 847 | 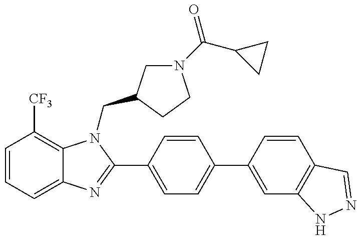 |
| 848 | 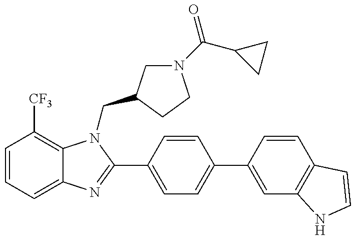 |
| 849 | 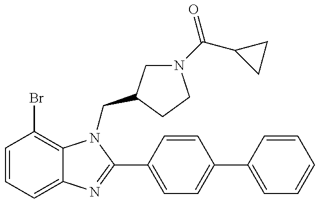 |
| 850 | 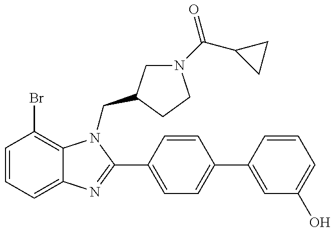 |
| 851 | 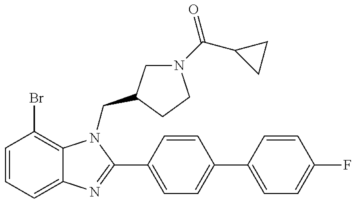 |
| 852 | 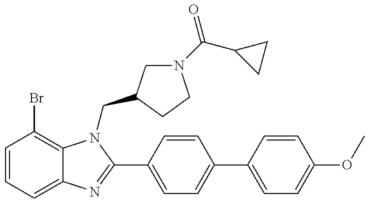 |
| 853 | 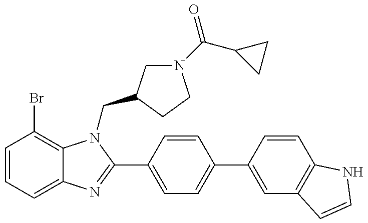 |
| 854 | 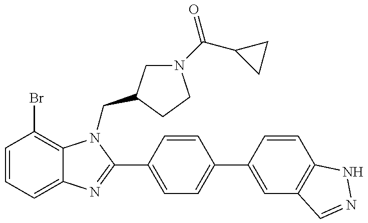 |
| 855 | 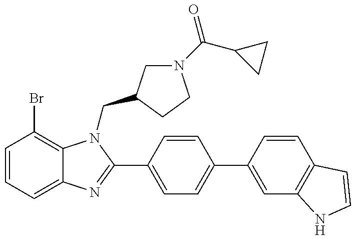 |
| 856 | 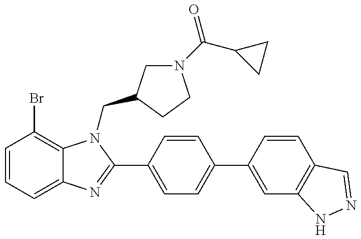 |
| 857 | 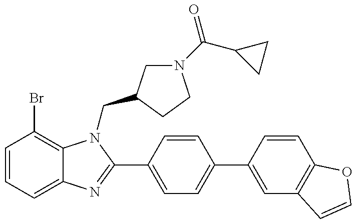 |
| 858 | 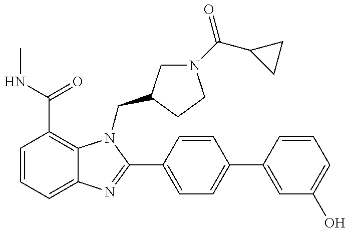 |
| 859 | 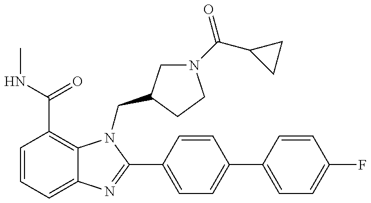 |
| 860 | 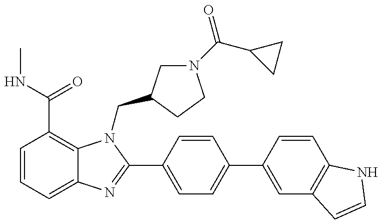 |
| 861 | 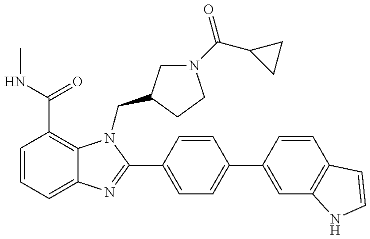 |
| 862 | 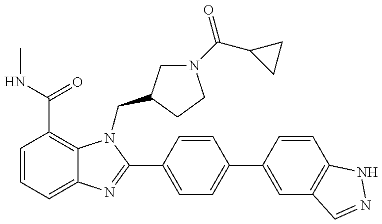 |
| 863 | 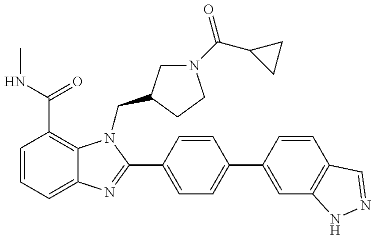 |
| 864 | 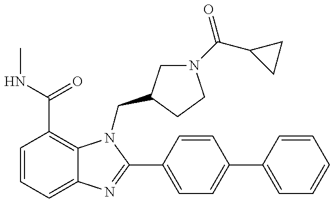 |
| 865 | 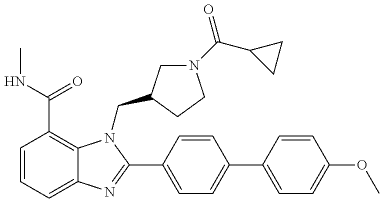 |
| 866 | 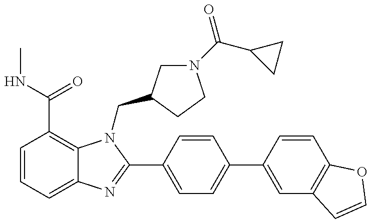 |
| 867 | 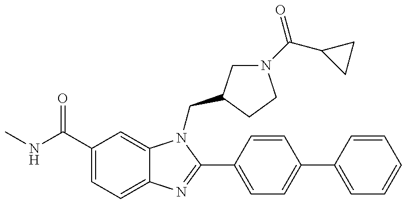 |
| 868 | 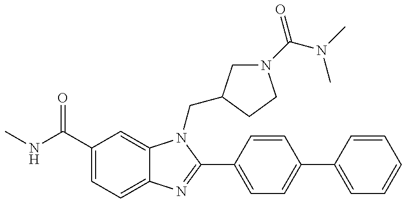 [SMILES: CNC(=O)c1ccc2nc(-c3ccc(-c4ccccc4)cc3)n(CC3CCN(C(=O)N(C)C)C3)c2c1] |
| 869 | 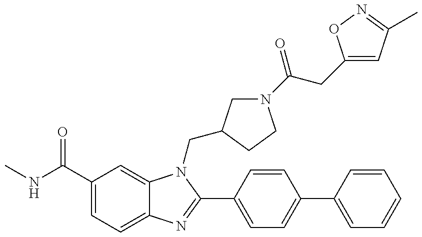 |
| 870 | 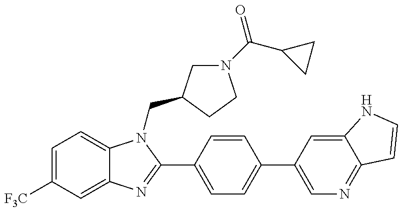 [SMILES: O=C(C1CC1)N1CC[C@@H](Cn2c(-c3ccc(-c4cnc5cc[nH]c5c4)cc3)nc3cc(C(F)(F)F)ccc32)C1] |
| 871 | 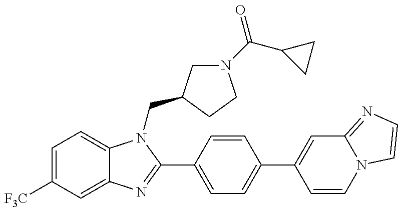 [SMILES: O=C(C1CC1)N1CC[C@@H](Cn2c(-c3ccc(-c4ccn5ccnc5c4)cc3)nc3cc(C(F)(F)F)ccc32)C1] |
| 872 | 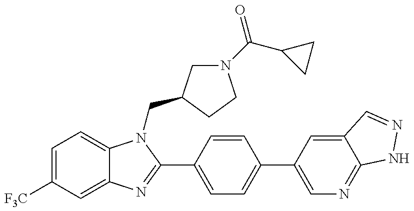 [SMILES: O=C(C1CC1)N1CC[C@@H](Cn2c(-c3ccc(-c4cnc5[nH]ncc5c4)cc3)nc3cc(C(F)(F)F)ccc32)C1] |
| 873 | 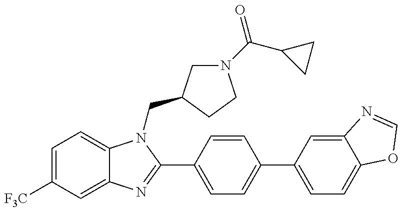 [SMILES: O=C(C1CC1)N1CC[C@@H](Cn2c(-c3ccc(-c4ccc5ocnc5c4)cc3)nc3cc(C(F)(F)F)ccc32)C1] |
| 874 | 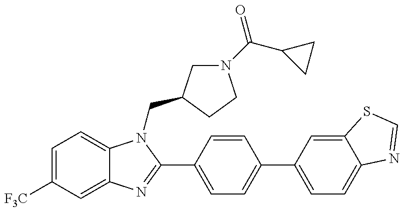 [SMILES: O=C(C1CC1)N1CC[C@@H](Cn2c(-c3ccc(-c4ccc5ncsc5c4)cc3)nc3cc(C(F)(F)F)ccc32)C1] |
| 875 | 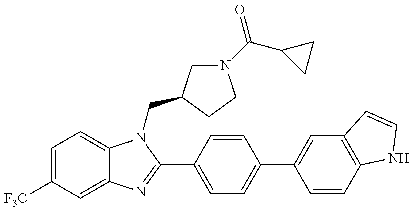 [SMILES: O=C(C1CC1)N1CC[C@@H](Cn2c(-c3ccc(-c4ccc5[nH]ccc5c4)cc3)nc3cc(C(F)(F)F)ccc32)C1] |
| 876 | 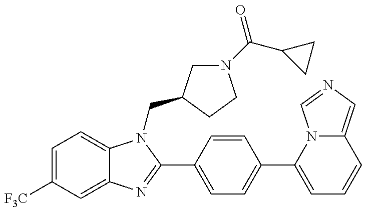 [SMILES: O=C(C1CC1)N1CC[C@@H](Cn2c(-c3ccc(-c4cccc5cncn45)cc3)nc3cc(C(F)(F)F)ccc32)C1] |
| 877 | 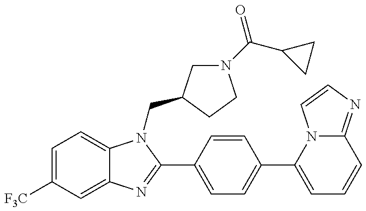 [SMILES: O=C(C1CC1)N1CC[C@@H](Cn2c(-c3ccc(-c4cccc5nccn45)cc3)nc3cc(C(F)(F)F)ccc32)C1] |
| 878 | 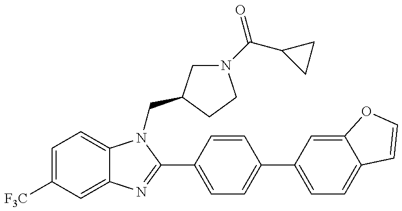 [SMILES: O=C(C1CC1)N1CC[C@@H](Cn2c(-c3ccc(-c4ccc5ccoc5c4)cc3)nc3cc(C(F)(F)F)ccc32)C1] |
| 879 | 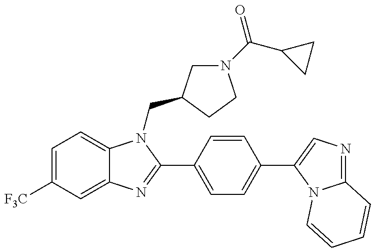 [SMILES: O=C(C1CC1)N1CC[C@@H](Cn2c(-c3ccc(-c4cnc5ccccn45)cc3)nc3cc(C(F)(F)F)ccc32)C1] |
| 880 | 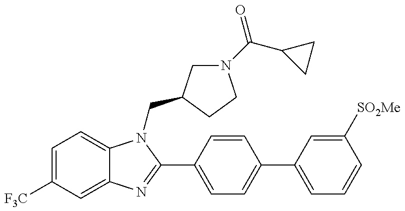 [SMILES: CS(=O)(=O)c1cccc(-c2ccc(-c3nc4cc(C(F)(F)F)ccc4n3C[C@@H]3CCN(C(=O)C4CC4)C3)cc2)c1] |
| 881 | 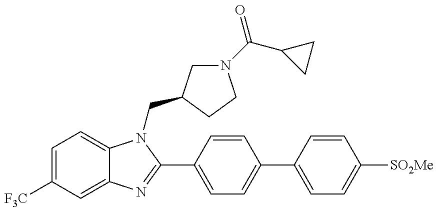 [SMILES: CS(=O)(=O)c1ccc(-c2ccc(-c3nc4cc(C(F)(F)F)ccc4n3C[C@@H]3CCN(C(=O)C4CC4)C3)cc2)cc1] |
| 882 | 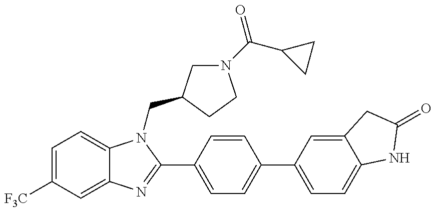 [SMILES: O=C1Cc2cc(-c3ccc(-c4nc5cc(C(F)(F)F)ccc5n4C[C@@H]4CCN(C(=O)C5CC5)C4)cc3)ccc2N1] |
| 883 | 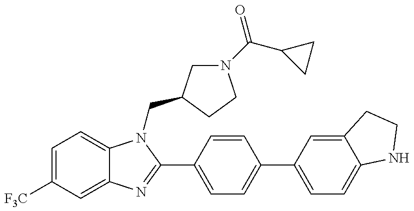 [SMILES: O=C(C1CC1)N1CC[C@@H](Cn2c(-c3ccc(-c4ccc5c(c4)CCN5)cc3)nc3cc(C(F)(F)F)ccc32)C1] |
| 884 | 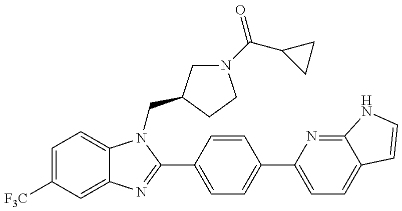 [SMILES: O=C(C1CC1)N1CC[C@@H](Cn2c(-c3ccc(-c4ccc5cc[nH]c5n4)cc3)nc3cc(C(F)(F)F)ccc32)C1] |
| 885 | 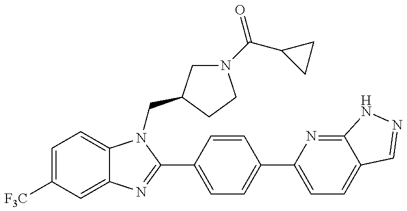 [SMILES: O=C(C1CC1)N1CC[C@@H](Cn2c(-c3ccc(-c4ccc5cn[nH]c5n4)cc3)nc3cc(C(F)(F)F)ccc32)C1] |
| 886 | 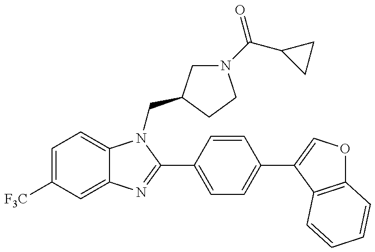 [SMILES: O=C(C1CC1)N1CC[C@@H](Cn2c(-c3ccc(-c4coc5ccccc45)cc3)nc3cc(C(F)(F)F)ccc32)C1] |
| 887 | 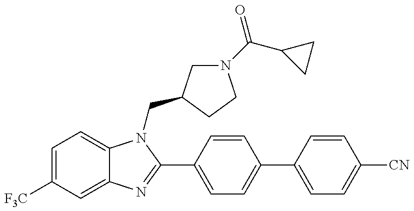 [SMILES: N#Cc1ccc(-c2ccc(-c3nc4cc(C(F)(F)F)ccc4n3C[C@@H]3CCN(C(=O)C4CC4)C3)cc2)cc1] |
| 888 | 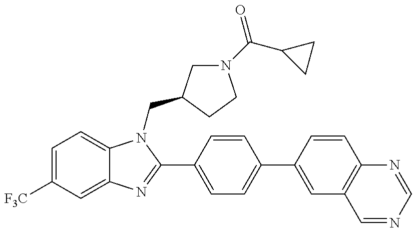 [SMILES: O=C(C1CC1)N1CC[C@@H](Cn2c(-c3ccc(-c4ccc5ncncc5c4)cc3)nc3cc(C(F)(F)F)ccc32)C1] |
| 889 | 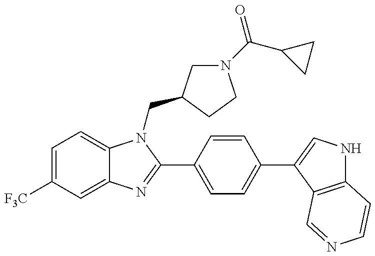 [SMILES: O=C(C1CC1)N1CC[C@@H](Cn2c(-c3ccc(-c4c[nH]c5ccncc45)cc3)nc3cc(C(F)(F)F)ccc32)C1] |
| 890 | 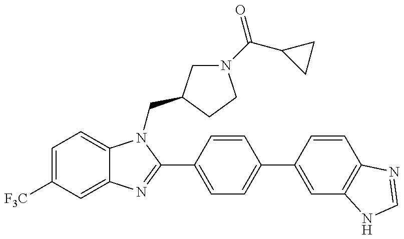 [SMILES: O=C(C1CC1)N1CC[C@@H](Cn2c(-c3ccc(-c4ccc5nc[nH]c5c4)cc3)nc3cc(C(F)(F)F)ccc32)C1] |
| 891 | 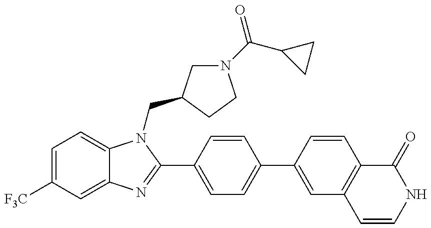 [SMILES: O=C(C1CC1)N1CC[C@@H](Cn2c(-c3ccc(-c4ccc5c(=O)[nH]ccc5c4)cc3)nc3cc(C(F)(F)F)ccc32)C1] |
| 892 | 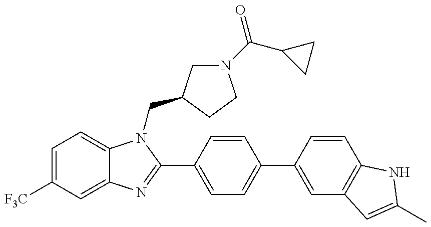 [SMILES: Cc1cc2cc(-c3ccc(-c4nc5cc(C(F)(F)F)ccc5n4C[C@@H]4CCN(C(=O)C5CC5)C4)cc3)ccc2[nH]1] |
| 893 | 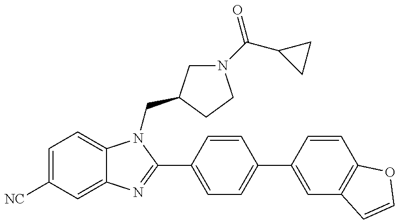 [SMILES: N#Cc1ccc2c(c1)nc(-c1ccc(-c3ccc4occc4c3)cc1)n2C[C@@H]1CCN(C(=O)C2CC2)C1] |
| 894 | 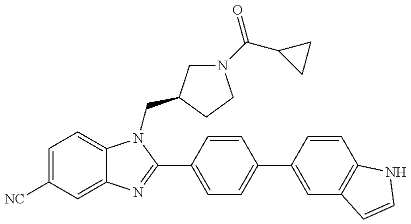 [SMILES: N#Cc1ccc2c(c1)nc(-c1ccc(-c3ccc4[nH]ccc4c3)cc1)n2C[C@@H]1CCN(C(=O)C2CC2)C1] |
| 895 | 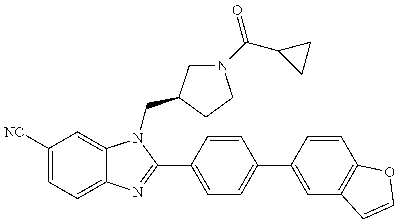 [SMILES: N#Cc1ccc2nc(-c3ccc(-c4ccc5occc5c4)cc3)n(C[C@@H]3CCN(C(=O)C4CC4)C3)c2c1] |
| 896 | 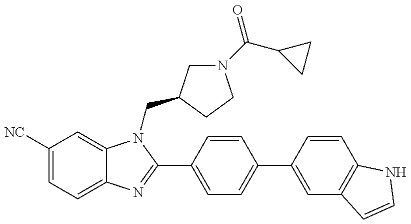 [SMILES: N#Cc1ccc2nc(-c3ccc(-c4ccc5[nH]ccc5c4)cc3)n(C[C@@H]3CCN(C(=O)C4CC4)C3)c2c1] |
| 897 | 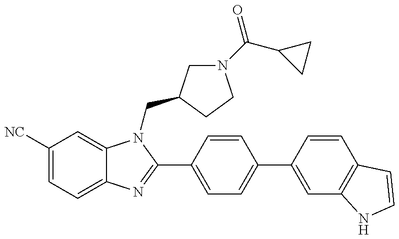 [SMILES: N#Cc1ccc2nc(-c3ccc(-c4ccc5cc[nH]c5c4)cc3)n(C[C@@H]3CCN(C(=O)C4CC4)C3)c2c1] |
| 898 | 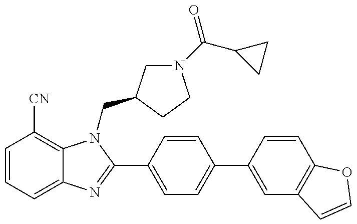 [SMILES: N#Cc1cccc2nc(-c3ccc(-c4ccc5occc5c4)cc3)n(C[C@@H]3CCN(C(=O)C4CC4)C3)c12] |
| 899 | 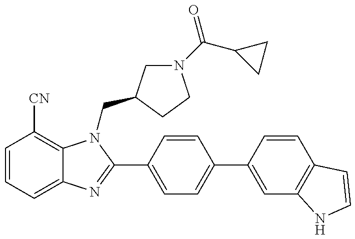 [SMILES: N#Cc1cccc2nc(-c3ccc(-c4ccc5cc[nH]c5c4)cc3)n(C[C@@H]3CCN(C(=O)C4CC4)C3)c12] |
| 900 | 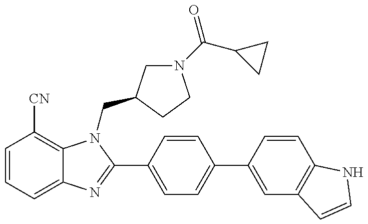 [SMILES: N#Cc1cccc2nc(-c3ccc(-c4ccc5[nH]ccc5c4)cc3)n(C[C@@H]3CCN(C(=O)C4CC4)C3)c12] |
| 901 | 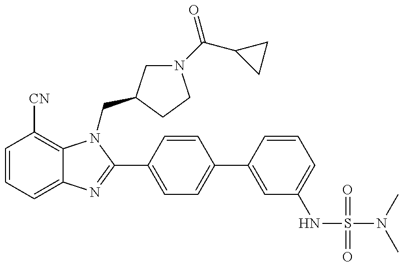 [SMILES: CN(C)S(=O)(=O)Nc1cccc(-c2ccc(-c3nc4cccc(C#N)c4n3C[C@@H]3CCN(C(=O)C4CC4)C3)cc2)c1] |
| 902 | 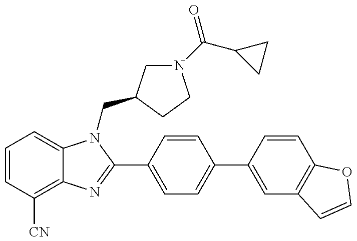 [SMILES: N#Cc1cccc2c1nc(-c1ccc(-c3ccc4occc4c3)cc1)n2C[C@@H]1CCN(C(=O)C2CC2)C1] |
| 903 | 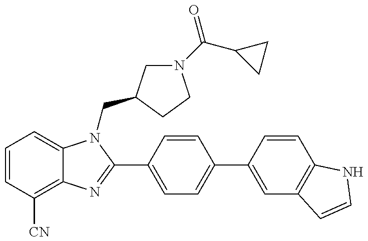 [SMILES: N#Cc1cccc2c1nc(-c1ccc(-c3ccc4[nH]ccc4c3)cc1)n2C[C@@H]1CCN(C(=O)C2CC2)C1] |
| 904 | 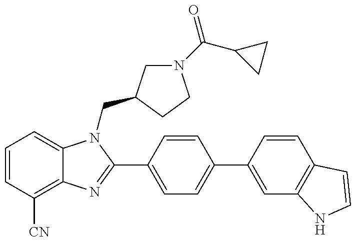 [SMILES: N#Cc1cccc2c1nc(-c1ccc(-c3ccc4cc[nH]c4c3)cc1)n2C[C@@H]1CCN(C(=O)C2CC2)C1] |
| 905 | 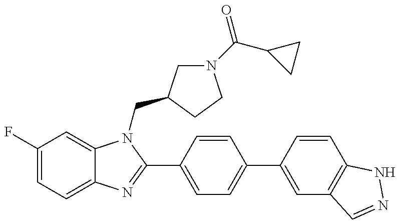 [SMILES: O=C(C1CC1)N1CC[C@@H](Cn2c(-c3ccc(-c4ccc5[nH]ncc5c4)cc3)nc3ccc(F)cc32)C1] |
| 906 | 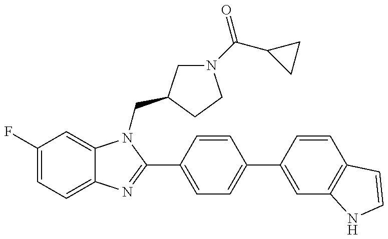 [SMILES: O=C(C1CC1)N1CC[C@@H](Cn2c(-c3ccc(-c4ccc5cc[nH]c5c4)cc3)nc3ccc(F)cc32)C1] |
| 907 | 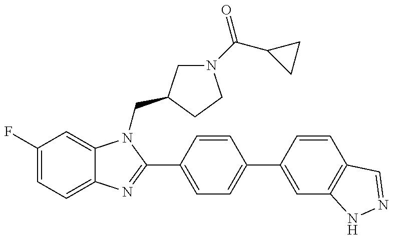 [SMILES: O=C(C1CC1)N1CC[C@@H](Cn2c(-c3ccc(-c4ccc5cn[nH]c5c4)cc3)nc3ccc(F)cc32)C1] |
| 908 | 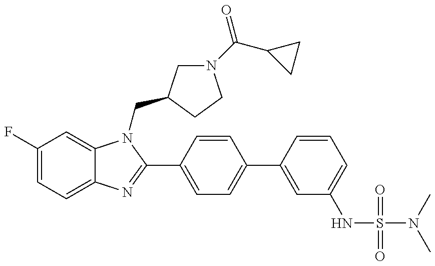 [SMILES: CN(C)S(=O)(=O)Nc1cccc(-c2ccc(-c3nc4ccc(F)cc4n3C[C@@H]3CCN(C(=O)C4CC4)C3)cc2)c1] |
| 909 | 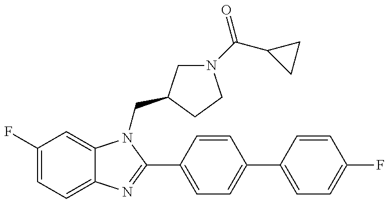 [SMILES: O=C(C1CC1)N1CC[C@@H](Cn2c(-c3ccc(-c4ccc(F)cc4)cc3)nc3ccc(F)cc32)C1] |
| 910 | 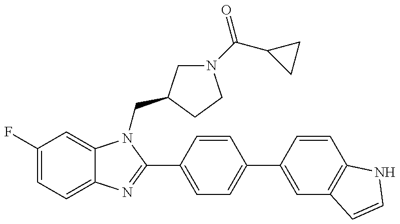 [SMILES: O=C(C1CC1)N1CC[C@@H](Cn2c(-c3ccc(-c4ccc5[nH]ccc5c4)cc3)nc3ccc(F)cc32)C1] |
| 911 | 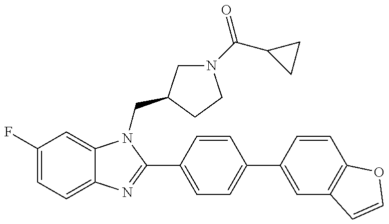 [SMILES: O=C(C1CC1)N1CC[C@@H](Cn2c(-c3ccc(-c4ccc5occc5c4)cc3)nc3ccc(F)cc32)C1] |
| 912 | 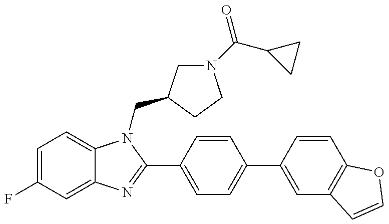 [SMILES: O=C(C1CC1)N1CC[C@@H](Cn2c(-c3ccc(-c4ccc5occc5c4)cc3)nc3cc(F)ccc32)C1] |
| 913 | 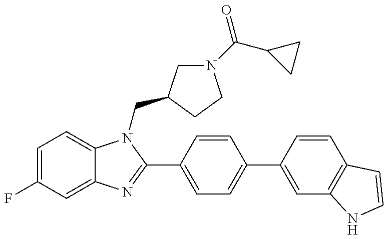 [SMILES: O=C(C1CC1)N1CC[C@@H](Cn2c(-c3ccc(-c4ccc5cc[nH]c5c4)cc3)nc3cc(F)ccc32)C1] |
| 914 | 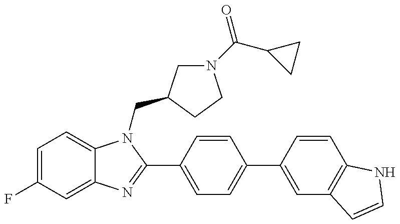 [SMILES: O=C(C1CC1)N1CC[C@@H](Cn2c(-c3ccc(-c4ccc5[nH]ccc5c4)cc3)nc3cc(F)ccc32)C1] |
| 915 | 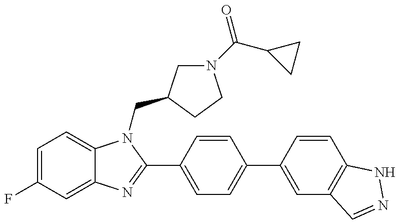 [SMILES: O=C(C1CC1)N1CC[C@@H](Cn2c(-c3ccc(-c4ccc5[nH]ncc5c4)cc3)nc3cc(F)ccc32)C1] |

In some embodiments, the FASN inhibitor is a compound of Formula (XXII):

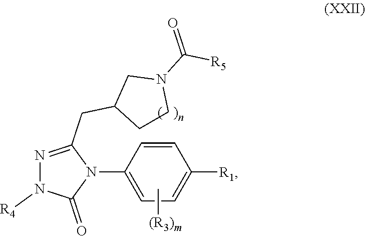

wherein, R₁ is a 6-membered aryl or heteroaryl ring which may be substituted or unsubstituted, in which adjacent substituents together may form an additional optionally substituted five or six membered ring which contains 0-3 hetero atoms and 0 to 2 double bonds; each R₃ is independently selected from the group consisting of: halogen, C₁₋₆ alkyl, hydroxyl and alkoxy; R₄ is H or C₁₋₆ alkyl; R₅ is selected from the group consisting of: C₁₋₆ alkyl, C₃₋₇ cycloalkyl, —OC₁₋₆ alkyl, C₄₋₆ heterocycloalkyl, amino, and alkylamino; m is 0, 1, 2, or 3; n is 0 or 1; or pharmaceutically acceptable salts thereof.

In some embodiments, the FASN inhibitor is a compound of Formula (XXII-A):

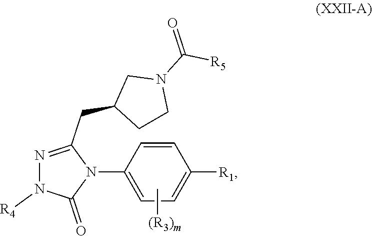

wherein, R₁ is a 6-membered aryl or heteroaryl ring which may be substituted or unsubstituted, in which adjacent substituents together may form an additional optionally substituted five or six membered ring which contains 0-3 hetero atoms and 0 to 2 double bonds; each R₃ is independently selected from the group consisting of: halogen, C₁₋₆ alkyl, hydroxyl and alkoxy; R₄ is H or C₁₋₆ alkyl; R₅ is selected from the group consisting of: C₁₋₆ alkyl, C₃₋₇ cycloalkyl, —OC₁₋₆ alkyl, C₄₋₆ heterocycloalkyl, amino and alkylamino; m is 0, 1, 2, or 3; or pharmaceutically acceptable salts thereof.

In some embodiments, the FASN inhibitor is a compound of Formula (XXII-B):

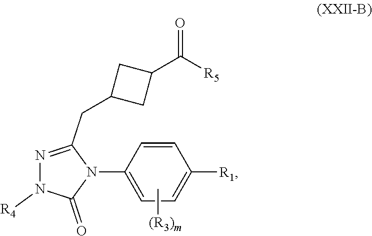

wherein, R₁ is a 6-membered aryl or heteroaryl ring which may be substituted or unsubstituted, in which adjacent substituents together may form an additional optionally substituted five or six membered ring which contains 0-3 hetero atoms and 0 to 2 double bonds; each R₃ is independently selected from the group consisting of: halogen, C₁₋₆ alkyl, hydroxyl and alkoxy; R₄ is H or C₁₋₆ alkyl; R₅ is selected from the group consisting of: C₁₋₆ alkyl, C₃₋₇ cycloalkyl, —OC₁₋₆ alkyl, C₄₋₆ heterocycloalkyl, amino and alkylamino;

m is 0, 1, 2, or 3; or pharmaceutically acceptable salts thereof.

In some embodiments, this invention also relates to compounds of Formula (XXII-A) or (XXII-B), wherein R₁ is a substituted or unsubstituted 6-membered aryl ring, in which adjacent substituents together may form an additional optionally substituted five or six membered ring which contains 0-3 hetero atoms and 0 to 2 double bonds; or pharmaceutically acceptable salts thereof. In some embodiments, this invention also relates to compounds of Formula (XXII-A) or (XXII-B), wherein R₁ is a substituted or unsubstituted 6-membered heteroaryl ring, in which adjacent substituents together may form an additional optionally substituted five or six membered ring which contains 0-3 hetero atoms and 0 to 2 double bonds; or pharmaceutically acceptable salts thereof. In some embodiments, this invention also relates to compounds of Formula (XXII-A) or (XXII-B), wherein R₁ is a substituted or unsubstituted pyridine or pyrimidine, in which adjacent substituents together may form an additional optionally substituted five or six membered ring which contains 0-3 hetero atoms and 0 to 2 double bonds; or pharmaceutically acceptable salts thereof. In some embodiments, this invention also relates to compounds of Formula (XXII-A) or (XXII-B), wherein R₁ is a 6-membered aryl optionally substituted by one to three substituents selected from the group consisting of: halogen, C₁₋₆ alkyl, alkoxy, hydroxyl, amino, substituted amino, sulfamide, and cyano, or pharmaceutically acceptable salts thereof. In some embodiments, this invention also relates to compounds of Formula (XXII-A) or (XXII-B), wherein R₁ is a 6-membered heteroaryl optionally substituted by one to three substituents selected from the group consisting of: halogen, C₁₋₆ alkyl, alkoxy, hydroxyl, amino, substituted amino, sulfamide, and cyano, or pharmaceutically acceptable salts thereof. In some embodiments, this invention also relates to compounds of Formula (XXII-A) or (XXII-B), wherein R₁ is an optionally substituted bicyclic ring selected from the group consisting of: benzimidazole, indole, benzofuran, dihydrobenzofuran, dihydroindole, imidazopyridine, quinoline, azaindole, isoquinoline, isoquinolone, quinazoline, naphthalene, dihydroindene, indene, and indazole; or pharmaceutically acceptable salts thereof.

In some embodiments, this invention also relates to compounds of any of the above embodiments, wherein R₃ is fluoro, chloro, hydroxyl, methoxy, or methyl, m is 0-1, or pharmaceutically acceptable salts thereof. In some embodiments, this invention also relates to compounds of any of the above embodiments, wherein R₄ is H, or pharmaceutically acceptable salts thereof. In some embodiments, this invention also relates to compounds of any of the above embodiments, wherein R₅ is cyclopropyl, methyl, ethyl or isopropyl, or pharmaceutically acceptable salts thereof. In some embodiments, this invention also relates to compounds of any of the above embodiments, wherein R₅ is cyclopropyl, or pharmaceutically acceptable salts thereof.

This invention also relates to the following compounds: 4-[4-(1-benzofuran-5-yl)phenyl]-5-{[(3S)-1-(cyclopropylcarbonyl)-3-pyrrolidinyl]methyl}-2,4-dihydro-3H-1,2,4-triazol-3-one, 5-{[(3S)-1-(cyclopropylcarbonyl)-3-pyrrolidinyl]methyl}-4-[4-(1H-indol-6-yl)phenyl]-2,4-dihydro-3H-1,2,4-triazol-3-one, 5-{[(3S)-1-(cyclopropylcarbonyl)-3-pyrrolidinyl]methyl}-4-[4′-(methyloxy)-4-biphenylyl]-2,4-dihydro-3H-1,2,4-triazol-3-one, 4′-(3-{[(3S)-1-(cyclopropylcarbonyl)-3-pyrrolidinyl]methyl}-5-oxo-1,5-dihydro-4H-1,2,4-triazol-4-yl)-4-biphenylcarbonitrile, 5-{[(3S)-1-(cyclopropylcarbonyl)-3-pyrrolidinyl]methyl}-4-(2′,4′-dichloro-4-biphenylyl)-2,4-dihydro-3H-1,2,4-triazol-3-one, 5-{[(3S)-1-(cyclopropylcarbonyl)-3-pyrrolidinyl]methyl}-4-(4-imidazo[1,2-a]pyridin-7-ylphenyl)-2,4-dihydro-3H-1,2,4-triazol-3-one, 4-[4-(1-benzofuran-5-yl)phenyl]-5-{[(3S)-1-(cyclopropylcarbonyl)-3-pyrrolidinyl]methyl}-2-methyl-2,4-dihydro-3H-1,2,4-triazol-3-one, (4-[4-(1-benzofuran-5-yl)phenyl]-3-{[(3S)-1-(cyclopropylcarbonyl)-3-pyrrolidinyl]methyl}-5-oxo-4,5-dihydro-1H-1,2,4-triazol-1-yl)acetic acid, 4-[4-(1-benzofuran-5-yl)phenyl]-5-{[(3S)-1-(cyclopropylcarbonyl)-3-pyrrolidinyl]methyl}-2-(1-methylethyl)-2,4-dihydro-3H-1,2,4-triazol-3-one, 4-[4-(1-benzofuran-5-yl)phenyl]-5-{[(3S)-1-(cyclopropylcarbonyl)-3-pyrrolidinyl]methyl}-2-[2-(methyloxy)ethyl]-2,4-dihydro-3H-1,2,4-triazol-3-one, methyl (4-[4-(1-benzofuran-5-yl)phenyl]-3-{[(3S)-1-(cyclopropylcarbonyl)-3-pyrrolidinyl]methyl}-5-oxo-4,5-dihydro-1H-1,2,4-triazol-1-yl)acetate, 4-[4-(1-benzofuran-5-yl)phenyl]-5-{[(3S)-1-(cyclopropylcarbonyl)-3-pyrrolidinyl]methyl}-2-(2-hydroxyethyl)-2,4-dihydro-3H-1,2,4-triazol-3-one, 5-{[(3S)-1-(cyclopropylcarbonyl)-3-pyrrolidinyl]methyl}-4-[4-(1H-indol-4-yl)phenyl]-2,4-dihydro-3H-1,2,4-triazol-3-one, 4-[4-(1-benzofuran-6-yl)phenyl]-5-{[(3S)-1-(cyclopropylcarbonyl)-3-pyrrolidinyl]methyl}-2,4-dihydro-3H-1,2,4-triazol-3-one, A′-[4′-(3-{[(3S)-1-(cyclopropylcarbonyl)-3-pyrrolidinyl]methyl}-5-oxo-1,5-dihydro-4H-1,2,4-triazol-4-yl)-3-biphenylyl]-N,N-dimethylsulfamide, 5-{[(3S)-1-(cyclopropylcarbonyl)-3-pyrrolidinyl]methyl}-4-[4-(3-methyl-1-benzofuran-5-yl)phenyl]-2,4-dihydro-3H-1,2,4-triazol-3-one, 5-{[(3S)-1-(cyclopropylcarbonyl)-3-pyrrolidinyl]methyl}-4-[4-(1H-indol-5-yl)phenyl]-2,4-dihydro-3H-1,2,4-triazol-3-one, 5-{[(3S)-1-(cyclopropylcarbonyl)-3-pyrrolidinyl]methyl}-4-[4-(2-methyl-1H-benzimidazol-5-yl)phenyl]-2,4-dihydro-3H-1,2,4-triazol-3-one, 5-{[(3S)-1-(cyclopropylcarbonyl)-3-pyrrolidinyl]methyl}-4-[4-(2,3-dihydro-1H-indol-5-yl)phenyl]-2,4-dihydro-3H-1,2,4-triazol-3-one, 5-{[(3S)-1-(cyclopropylcarbonyl)-3-pyrrolidinyl]methyl}-4-(4-imidazo[1,5-a]pyridin-5-ylphenyl)-2,4-dihydro-3H-1,2,4-triazol-3-one, 2-(4-[4-(1-benzofuran-5-yl)phenyl]-3-{[(3S)-1-(cyclopropylcarbonyl)-3-pyrrolidinyl]methyl}-5-oxo-4,5-dihydro-1H-1,2,4-triazol-1-yl)acetamide, (4-[4-(1-benzofuran-5-yl)phenyl]-3-{[(3S)-1-(cyclopropylcarbonyl)-3-pyrrolidinyl]methyl}-5-oxo-4,5-dihydro-1H-1,2,4-triazol-1-yl)acetonitrile, 5-{[(3S)-1-(cyclopropylcarbonyl)-3-pyrrolidinyl]methyl}-4-[4-(1-methyl-1H-benzimidazol-5-yl)phenyl]-2,4-dihydro-3H-1,2,4-triazol-3-one, 5-{[(3S)-1-(cyclopropylcarbonyl)-3-pyrrolidinyl]methyl}-4-[4-(1-methyl-1H-indol-5-yl)phenyl]-2,4-dihydro-3H-1,2,4-triazol-3-one, 2-(2-aminoethyl)-4-[4-(1-benzofuran-5-yl)phenyl]-5-{[(3S)-1-(cyclopropylcarbonyl)-3-pyrrolidinyl]methyl}-2,4-dihydro-3H-1,2,4-triazol-3-one, 5-{[(3S)-1-(cyclopropylcarbonyl)-3-pyrrolidinyl]methyl}-4-[4-(3-methyl-1H-indol-5-yl)phenyl]-2,4-dihydro-3H-1,2,4-triazol-3-one, 5-{[(3S)-1-(cyclopropylcarbonyl)-3-pyrrolidinyl]methyl}-4-[4-(2-methyl-1-benzofuran-5-yl)phenyl]-2,4-dihydro-3H-1,2,4-triazol-3-one, 5-{[(3S)-1-(cyclopropylcarbonyl)-3-pyrrolidinyl]methyl}-4-[4-(2-methyl-1H-indol-5-yl)phenyl]-2,4-dihydro-3H-1,2,4-triazol-3-one, 4-[4-(1-benzofuran-5-yl)phenyl]-5-{[(3S)-1-(cyclopropylcarbonyl)-3-pyrrolidinyl]methyl}-2-(2-hydroxy-2-methylpropyl)-2,4-dihydro-3H-1,2,4-triazol-3-one, 5-{[(3S)-1-(cyclopropylcarbonyl)-3-pyrrolidinyl]methyl}-4-[2-fluoro-4-(1H-indol-6-yl)phenyl]-2,4-dihydro-3H-1,2,4-triazol-3-one, 5-{[(3S)-1-(cyclopropylcarbonyl)-3-pyrrolidinyl]methyl}-4-[2-fluoro-4-(1H-indol-5-yl)phenyl]-2,4-dihydro-3H-1,2,4-triazol-3-one, 4-[4-(1-benzofuran-5-yl)-2-fluorophenyl]-5-{[(3S)-1-(cyclopropylcarbonyl)-3-pyrrolidinyl]methyl}-2,4-dihydro-3H-1,2,4-triazol-3-one, 5-{[(3S)-1-(cyclopropylcarbonyl)-3-pyrrolidinyl]methyl}-4-[4-(7-methyl-1-benzofuran-5-yl)phenyl]-2,4-dihydro-3H-1,2,4-triazol-3-one, 5-{[(3S)-1-(cyclopropylcarbonyl)-3-pyrrolidinyl]methyl}-4-(4′-fluoro-4-biphenylyl)-2,4-dihydro-3H-1,2,4-triazol-3-one, 5-{[(3S)-1-(cyclopropylcarbonyl)-3-pyrrolidinyl]methyl}-4-[4-(6-quinolinyl)phenyl]-2,4-dihydro-3H-1,2,4-triazol-3-one, 5-{[(3S)-1-(cyclopropylcarbonyl)-3-pyrrolidinyl]methyl}-4-[4-(1H-indazol-5-yl)phenyl]-2,4-dihydro-3H-1,2,4-triazol-3-one, 5-{[(3S)-1-(cyclopropylcarbonyl)-3-pyrrolidinyl]methyl}-4-[4-(2,3-dihydro-1-benzofuran-5-yl)phenyl]-2,4-dihydro-3H-1,2,4-triazol-3-one, 4-[4-(1,3-benzodioxol-5-yl)phenyl]-5-{[(3S)-1-(cyclopropylcarbonyl)-3-pyrrolidinyl]methyl}-2,4-dihydro-3H-1,2,4-triazol-3-one, 5-{[(3S)-1-(cyclopropylcarbonyl)-3-pyrrolidinyl]methyl}-4-[4′-(dimethylamino)-4-biphenylyl]-2,4-dihydro-3H-1,2,4-triazol-3-one, 4-[4-(1-benzofuran-5-yl)-2-methylphenyl]-5-{[(3S)-1-(cyclopropylcarbonyl)-3-pyrrolidinyl]methyl}-2,4-dihydro-3H-1,2,4-triazol-3-one, 5-{[(3S)-1-(cyclopropylcarbonyl)-3-pyrrolidinyl]methyl}-4-[4-(1H-indol-5-yl)-2-methylphenyl]-2,4-dihydro-3H-1,2,4-triazol-3-one, 5-{[(3S)-1-(cyclopropylcarbonyl)-3-pyrrolidinyl]methyl}-4-[4-(1H-indol-6-yl)-2-methylphenyl]-2,4-dihydro-3H-1,2,4-triazol-3-one, 5-{[(3S)-1-(cyclopropylcarbonyl)-3-pyrrolidinyl]methyl}-4-(4′-fluoro-3-methyl-4-biphenylyl)-2,4-dihydro-3H-1,2,4-triazol-3-one, 4-[4-(1-benzofuran-5-yl)phenyl]-5-{[(3S)-1-propanoyl-3-pyrrolidinyl]methyl}-2,4-dihydro-3H-1,2,4-triazol-3-one, 4-[4-(1-benzofuran-5-yl)phenyl]-5-{[(3S)-1-(2-methylpropanoyl)-3-pyrrolidinyl]methyl}-2,4-dihydro-3H-1,2,4-triazol-3-one, 4-(4′-amino-4-biphenylyl)-5-{[(3S)-1-(cyclopropylcarbonyl)-3-pyrrolidinyl]methyl}-2,4-dihydro-3H-1,2,4-triazol-3-one, 4-[4-(6-amino-3-pyridinyl)phenyl]-5-{[(3S)-1-(cyclopropylcarbonyl)-3-pyrrolidinyl]methyl}-2,4-dihydro-3H-1,2,4-triazol-3-one, 5-{[(3S)-1-(cyclopropylcarbonyl)-3-pyrrolidinyl]methyl}-4-(3,3′-difluoro-4′-methyl-4-biphenylyl)-2,4-dihydro-3H-1,2,4-triazol-3-one, 4-(3′-chloro-3-fluoro-4′-methyl-4-biphenylyl)-5-{[(3S)-1-(cyclopropylcarbonyl)-3-pyrrolidinyl]methyl}-2,4-dihydro-3H-1,2,4-triazol-3-one, 4-[4-(1-benzofuran-5-yl)-2,6-difluorophenyl]-5-{[(3S)-1-(cyclopropylcarbonyl)-3-pyrrolidinyl]methyl}-2,4-dihydro-3H-1,2,4-triazol-3-one, 5-{[(3S)-1-(cyclopropylcarbonyl)-3-pyrrolidinyl]methyl}-4-[2,6-difluoro-4-(7-quinolinyl)phenyl]-2,4-dihydro-3H-1,2,4-triazol-3-one, 4′-(3-{[(3S)-1-(cyclopropylcarbonyl)-3-pyrrolidinyl]methyl}-5-oxo-1,5-dihydro-4H-1,2,4-triazol-4-yl)-3′,5′-difluoro-3-methyl-4-biphenylcarbonitrile, 5-{[(3S)-1-(cyclopropylcarbonyl)-3-pyrrolidinyl]methyl}-4-[2,6-difluoro-4-(1H-indol-6-yl)phenyl]-2,4-dihydro-3H-1,2,4-triazol-3-one, 5-{[(3S)-1-(cyclopropylcarbonyl)-3-pyrrolidinyl]methyl}-4-[3,5-difluoro-4′-(methyloxy)-4-biphenylyl]-2,4-dihydro-3H-1,2,4-triazol-3-one, 4-(4′-chloro-2′,3,5-trifluoro-4-biphenylyl)-5-{[(3S)-1-(cyclopropylcarbonyl)-3-pyrrolidinyl]methyl}-2,4-dihydro-3H-1,2,4-triazol-3-one, 4-(4′-chloro-3,5-difluoro-4-biphenylyl)-5-{[(3S)-1-(cyclopropylcarbonyl)-3-pyrrolidinyl]methyl}-2,4-dihydro-3H-1,2,4-triazol-3-one, 4-[4-(1-benzofuran-5-yl)-3-(methyloxy)phenyl]-5-{[(3S)-1-(cyclopropylcarbonyl)-3-pyrrolidinyl]methyl}-2,4-dihydro-3H-1,2,4-triazol-3-one, 4-[4-(1-benzofuran-5-yl)-2-(trifluoromethyl)phenyl]-5-{[(3S)-1-(cyclopropylcarbonyl)-3-pyrrolidinyl]methyl}-2,4-dihydro-3H-1,2,4-triazol-3-one, 4-[4-(1-benzofuran-5-yl)-2-hydroxyphenyl]-5-{[(3S)-1-(cyclopropylcarbonyl)-3-pyrrolidinyl]methyl}-2,4-dihydro-3H-1,2,4-triazol-3-one, 4-[4-(1-benzofuran-5-yl)-2,5-difluorophenyl]-5-{[(3S)-1-(cyclopropylcarbonyl)-3-pyrrolidinyl]methyl}-2,4-dihydro-3H-1,2,4-triazol-3-one, 5-{[(3S)-1-(cyclopropylcarbonyl)-3-pyrrolidinyl]methyl}-4-[2,5-difluoro-4-(7-quinolinyl)phenyl]-2,4-dihydro-3H-1,2,4-triazol-3-one, 4′-(3-{[(3S)-1-(cyclopropylcarbonyl)-3-pyrrolidinyl]methyl}-5-oxo-1,5-dihydro-4H-1,2,4-triazol-4-yl)-2′,5′-difluoro-3-methyl-4-biphenylcarbonitrile, 5-{[(3S)-1-(cyclopropylcarbonyl)-3-pyrrolidinyl]methyl}-4-[2,5-difluoro-4-(1H-indol-6-yl)phenyl]-2,4-dihydro-3H-1,2,4-triazol-3-one, 4-[4-(1-benzofuran-5-yl)-2,3-difluorophenyl]-5-{[(3S)-1-(cyclopropylcarbonyl)-3-pyrrolidinyl]methyl}-2,4-dihydro-3H-1,2,4-triazol-3-one, 5-{[(3S)-1-(cyclopropylcarbonyl)-3-pyrrolidinyl]methyl}-4-[2,3-difluoro-4-(7-quinolinyl)phenyl]-2,4-dihydro-3H-1,2,4-triazol-3-one, 5-{[I-(cyclopropylcarbonyl)-3-azetidinyl]methyl}-4-[4-(1H-indazol-6-yl)phenyl]-2,4-dihydro-3H-1,2,4-triazol-3-one, 5-{[I-(cyclopropylcarbonyl)-3-azetidinyl]methyl}-4-[4-(1H-indol-6-yl)phenyl]-2,4-dihydro-3H-1,2,4-triazol-3-one, 5-{[1-(cyclopropylcarbonyl)-3-azetidinyl]methyl}-4-[4-(6-quinolinyl)phenyl]-2,4-dihydro-3H-1,2,4-triazol-3-one, 5-{[1-(cyclopropylcarbonyl)-3-azetidinyl]methyl}-4-[4′-(methyloxy)-4-biphenylyl]-2,4-dihydro-3H-1,2,4-triazol-3-one, 5-{[1-(cyclopropylcarbonyl)-3-azetidinyl]methyl}-4-[4′-(dimethylamino)-4-biphenylyl]-2,4-dihydro-3H-1,2,4-triazol-3-one, 4-[4-(1-benzofuran-5-yl)phenyl]-5-{[1-(cyclopropylcarbonyl)-3-azetidinyl]methyl}-2,4-dihydro-3H-1,2,4-triazol-3-one, 5-{[1-(cyclopropylcarbonyl)-3-azetidinyl]methyl}-4-[2-fluoro-4-(1H-indol-6-yl)phenyl]-2,4-dihydro-3H-1,2,4-triazol-3-one, 4′-(3-{[1-(cyclopropylcarbonyl)-3-azetidinyl]methyl}-5-oxo-1,5-dihydro-4H-1,2,4-triazol-4-yl)-3′-fluoro-4-biphenylcarbonitrile, 5-{[(3S)-1-(cyclopropylcarbonyl)-3-pyrrolidinyl]methyl}-4-[3′-(phenylcarbonyl)-4-biphenylyl]-2,4-dihydro-3H-1,2,4-triazol-3-one, 5-{[(3S)-1-(cyclopropylcarbonyl)-3-pyrrolidinyl]methyl}-4-[3-fluoro-3′-(phenylcarbonyl)-4-biphenylyl]-2,4-dihydro-3H-1,2,4-triazol-3-one, 4-[2-chloro-4-(1H-indol-5-yl)phenyl]-5-{[(3S)-1-(cyclopropylcarbonyl)-3-pyrrolidinyl]methyl}-2,4-dihydro-3H-1,2,4-triazol-3-one, 4-[2-chloro-4-(1H-indazol-5-yl)phenyl]-5-{[(3S)-1-(cyclopropylcarbonyl)-3-pyrrolidinyl]methyl}-2,4-dihydro-3H-1,2,4-triazol-3-one, 4-[4-(1-benzofuran-5-yl)-2-chlorophenyl]-5-{[(3S)-1-(cyclopropylcarbonyl)-3-pyrrolidinyl]methyl}-2,4-dihydro-3H-1,2,4-triazol-3-one, 4-[2-chloro-4-(1H-indol-6-yl)phenyl]-5-{[(3S)-1-(cyclopropylcarbonyl)-3-pyrrolidinyl]methyl}-2,4-dihydro-3H-1,2,4-triazol-3-one, 5-{[(3S)-1-(cyclopropylcarbonyl)-3-pyrrolidinyl]methyl}-4-[4′-fluoro-3-(methyloxy)-4-biphenylyl]-2,4-dihydro-3H-1,2,4-triazol-3-one, 5-{[(3S)-1-(cyclopropylcarbonyl)-3-pyrrolidinyl]methyl}-4-[4-(1H-indol-5-yl)-2-(methyloxy)phenyl]-2,4-dihydro-3H-1,2,4-triazol-3-one, 4-[4-(1-benzofuran-5-yl)-2-(methyloxy)phenyl]-5-{[(3S)-1-(cyclopropylcarbonyl)-3-pyrrolidinyl]methyl}-2,4-dihydro-3H-1,2,4-triazol-3-one, 5-{[(3S)-1-(cyclopropylcarbonyl)-3-pyrrolidinyl]methyl}-4-[4-(1H-indol-6-yl)-2-(methyloxy)phenyl]-2,4-dihydro-3H-1,2,4-triazol-3-one, 4′-(3-{[(3S)-1-(cyclopropylcarbonyl)-3-pyrrolidinyl]methyl}-5-oxo-1,5-dihydro-4H-1,2,4-triazol-4-yl)-3′-(methyloxy)-4-biphenylcarbonitrile, 5-{[(3S)-1-(cyclopropylcarbonyl)-3-pyrrolidinyl]methyl}-4-[4-(7-fluoro-1-benzofuran-5-yl)phenyl]-2,4-dihydro-3H-1,2,4-triazol-3-one, 4-[4-(2,1,3-benzoxadiazol-5-yl)phenyl]-5-{[(3S)-1-(cyclopropylcarbonyl)-3-pyrrol idinyl]methyl}-2,4-dihydro-3H-1,2,4-triazol-3-one, 5-{[(3S)-1-(cyclopropylcarbonyl)-3-pyrrol idinyl]methyl}-4-[2-fluoro-4-(1H-indazol-5-yl)phenyl]-2,4-dihydro-3H-1,2,4-triazol-3-one, 5-{[(3S)-1-(cyclopropylcarbonyl)-3-pyrrolidinyl]methyl}-4-[2-fluoro-4-(6-quinolinyl)phenyl]-2,4-dihydro-3H-1,2,4-triazol-3-one, 5-[4-(3-{[(3S)-1-(cyclopropylcarbonyl)-3-pyrrolidinyl]methyl}-5-oxo-1,5-dihydro-4H-1,2,4-triazol-4-yl)phenyl]-1,3-dihydro-2H-indol-2-one, 5-{[(3S)-1-(cyclopropylcarbonyl)-3-pyrrol idinyl]methyl}-4-[4-(1H-indazol-6-yl)phenyl]-2,4-dihydro-3H-1,2,4-triazol-3-one, 7-[4-(3-{[(3S)-1-(cyclopropylcarbonyl)-3-pyrrolidinyl]methyl}-5-oxo-1,5-dihydro-4H-1,2,4-triazol-4-yl)phenyl]-1 (2H)-isoquinolinone, 5-{[(3S)-1-(cyclopropylcarbonyl)-3-pyrrolidinyl]methyl}-4-[4-(2,3-dihydro-1-benzofuran-5-yl)-2-fluorophenyl]-2,4-dihydro-3H-1,2,4-triazol-3-one, 5-{[(3S)-1-(cyclopropylcarbonyl)-3-pyrrolidinyl]methyl}-4-[4-(2,3-dihydro-1H-indol-5-yl)-2-fluorophenyl]-2,4-dihydro-3H-1,2,4-triazol-3-one, 4-[4-(1,3-benzothiazol-5-yl)phenyl]-5-{[(3S)-1-(cyclopropylcarbonyl)-3-pyrrolidinyl]methyl}-2,4-dihydro-3H-1,2,4-triazol-3-one, 5-{[(3S)-1-(cyclopropylcarbonyl)-3-pyrrol idinyl]methyl}-4-{4′-[(dimethylamino)methyl]-4-biphenylyl}-2,4-dihydro-3H-1,2,4-triazol-3-one, 5-{[(3S)-1-(cyclopropylcarbonyl)-3-pyrrolidinyl]methyl}-4-(3′-fluoro-4-biphenylyl)-2,4-dihydro-3H-1,2,4-triazol-3-one, 5-{[(3S)-1-(cyclopropylcarbonyl)-3-pyrrolidinyl]methyl}-4-[3′-(methyloxy)-4-biphenylyl]-2,4-dihydro-3H-1,2,4-triazol-3-one, 5-{[(3S)-1-(cyclopropylcarbonyl)-3-pyrrolidinyl]methyl}-4-[3′-(dimethyl amino)-4-biphenylyl]-2,4-dihydro-3H-1,2,4-triazol-3-one, 5-{[(3S)-1-(cyclopropylcarbonyl)-3-pyrrolidinyl]methyl}-4-[4′-(hydroxymethyl)-4-biphenylyl]-2,4-dihydro-3H-1,2,4-triazol-3-one, 5-{[(3S)-1-(cyclopropylcarbonyl)-3-pyrrolidinyl]methyl}-4-[3′-(hydroxymethyl)-4-biphenylyl]-2,4-dihydro-3H-1,2,4-triazol-3-one, 4-[4-(1,3-benzoxazol-5-yl)phenyl]-5-{[(3S)-1-(cyclopropylcarbonyl)-3-pyrrolidinyl]methyl}-2,4-dihydro-3H-1,2,4-triazol-3-one, 5-{[(3S)-1-(cyclopropylcarbonyl)-3-pyrrolidinyl]methyl}-4-[4′-(1H-pyrazol-1-yl)-4-biphenylyl]-2,4-dihydro-3H-1,2,4-triazol-3-one, 5-{[(3S)-1-(cyclopropylcarbonyl)-3-pyrrolidinyl]methyl}-4-[3′-(1H-pyrazol-5-yl)-4-biphenylyl]-2,4-dihydro-3H-1,2,4-triazol-3-one, 4-[4-(1,3-benzothiazol-5-yl)-2-fluorophenyl]-5-{[(3S)-1-(cyclopropylcarbonyl)-3-pyrrolidinyl]methyl}-2,4-dihydro-3H-1,2,4-triazol-3-one, 5-{[(3S)-1-(cyclopropylcarbonyl)-3-pyrrolidinyl]methyl}-4-[4-(2-naphthalenyl)phenyl]-2,4-dihydro-3H-1,2,4-triazol-3-one, 5-{[(3S)-1-(cyclopropylcarbonyl)-3-pyrrolidinyl]methyl}-4-[3′-(1H-pyrazol-1-yl)-4-biphenylyl]-2,4-dihydro-3H-1,2,4-triazol-3-one, 5-{[(3S)-1-(cyclopropylcarbonyl)-3-pyrrol idinyl]methyl}-4-[4′-(1H-pyrazol-5-yl)-4-biphenylyl]-2,4-dihydro-3H-1,2,4-triazol-3-one, 4-[4-(1,3-benzothiazol-6-yl)phenyl]-5-{[(3S)-1-(cyclopropylcarbonyl)-3-pyrrolidinyl]methyl}-4-{3′-[(dimethylamino)methyl]-4-biphenylyl}-2,4dihydro-3H-1,2,4-triazol-3-one, 5-{[(3S)-1-(cyclopropylcarbonyl)-3-pyrrolidinyl]methyl}-4-(3,4′-difluoro-4-biphenylyl)-2,4-dihydro-3H-1,2,4-triazol-3-one, 4′-(3-{[(3S)-1-(cyclopropylcarbonyl)-3-pyrrolidmyl]methyl}-5-oxo-1,5-dihydro-4H-1,2,4-triazol-4-yl)-3′-fluoro-4-biphenylcarbonitrile, 5-{[(3S)-1-(cyclopropylcarbonyl)-3-pyrrolidinyl]methyl}-4-[4′-(dimethylamino)-3-fluoro-4-biphenylyl]-2,4-dihydro-3H-1,2,4-triazol-3-one, 4′-(3-{[(3S)-1-(cyclopropylcarbonyl)-3-pyrrolidmyl]methyl}-5-oxo-1,5-dihydro-4H-1,2,4-triazol-4-yl)-3,3′-difluoro-4-biphenylcarbonitrile, 5-{[(3S)-1-(cyclopropylcarbonyl)-3-pyrrolidinyl]methyl}-4-[3-fluoro-4′-(1H-pyrazol-1-yl)-4-biphenylyl]-2,4-dihydro-3H-1,2,4-triazol-3-one, 5-{[(3S)-1-(cyclopropylcarbonyl)-3-pyrrolidinyl]methyl}-4-[2-fluoro-4-(5-quinolinyl)phenyl]-2,4-dihydro-3H-1,2,4-triazol-3-one, 5-{[(3S)-1-(cyclopropylcarbonyl)-3-pyrrolidinyl]methyl}-4-[3-fluoro-4′-(methyloxy)-4-biphenylyl]-2,4-dihydro-3H-1,2,4-triazol-3-one, 4′-(3-{[(3S)-1-(cyclopropylcarbonyl)-3-pyrrolidmyl]methyl}-5-oxo-1,5-dihydro-4H-1,2,4-triazol-4-yl)-3′-fluoro-3-methyl-4-biphenylcarbonitrile, 4′-(3-{[(3S)-1-(cyclopropylcarbonyl)-3-pyrrolidmyl]methyl}-5-oxo-1,5-dihydro-4H-1,2,4-triazol-4-yl)-3′-fluoro-3-(methyloxy)-4-biphenylcarbonitrile, 5-{[(3S)-1-(cyclopropylcarbonyl)-3-pyrrolidinyl]methyl}-4-[2-fluoro-4-(6-quinoxalinyl)phenyl]-2,4-dihydro-3H-1,2,4-triazol-3-one, 5-{[(3S)-1-(cyclopropylcarbonyl)-3-pyrrolidmyl]methyl}-4-[4-(1-methyl-1H-indol-6-yl)phenyl]-2,4-dihydro-3H-1,2,4-triazol-3-one, 5-{[(3S)-1-(cyclopropylcarbonyl)-3-pyrrolidinyl]methyl}-4-[2-fluoro-4-(6-quinazolinyl)phenyl]-2,4-dihydro-3H-1,2,4-triazol-3-one, 5-{[(3S)-1-(cyclopropylcarbonyl)-3-pyrrolidinyl]methyl}-4-[2-fluoro-4-(2-methyl-6-quinolinyl)phenyl]-2,4-dihydro-3H-1,2,4-triazol-3-one, 5-{[(3S)-1-(cyclopropylcarbonyl)-3-pyrrolidinyl]methyl}-4-[4-(1-naphthalenyl)phenyl]-2,4-dihydro-3H-1,2,4-triazol-3-one, 5-{[(3S)-1-(cyclopropylcarbonyl)-3-pyrrolidinyl]methyl}-4-[2-fluoro-4-(7-quinolinyl)phenyl]-2,4-dihydro-3H-1,2,4-triazol-3-one, 5-{[(3S)-1-(cyclopropylcarbonyl)-3-pyrrolidinyl]methyl}-4-(1,Γ:4′,1″-terphenyl-4-yl)-2,4-dihydro-3H-1,2,4-triazol-3-one, 5-{[(3S)-1-(cyclopropylcarbonyl)-3-pyrrolidinyl]methyl}-4-[4-(3-quinolinyl)phenyl]-2,4-dihydro-3H-1,2,4-triazol-3-one, 5-{[(3S)-1-(cyclopropylcarbonyl)-3-pyrrolidinyl]methyl}-4-[3,3′-difluoro-4′-(1H-pyrazol-1-yl)-4-biphenylyl]-2,4-dihydro-3H-1,2,4-triazol-3-one, 4′-(3-{[(3S)-1-(cyclopropylcarbonyl)-3-pyrrolidinyl]methyl}-5-oxo-1,5-dihydro-4H-1,2,4-triazol-4-yl)-2,3′-difluoro-4-biphenylcarbonitrile, 4′-(3-{[(3S)-1-(cyclopropylcarbonyl)-3-pyrrolidinyl]methyl}-5-oxo-1,5-dihydro-4H-1,2,4-triazol-4-yl)-3′-fluoro-2-methyl-4-biphenylcarbonitrile, 3-chloro-4′-(3-{[(3S)-1-(cyclopropylcarbonyl)-3-pyrrolidinyl]methyl}-5-oxo-1,5-dihydro-4H-1,2,4-triazol-4-yl)-3′-fluoro-4-biphenylcarbonitrile, 5-{[(3S)-1-(cyclopropylcarbonyl)-3-pyrrolidinyl]methyl}-4-[4-(6-hydroxy-2-naphthalenyl)phenyl]-2,4-dihydro-3H-1,2,4-triazol-3-one, 5-{[(3S)-1-(cyclopropylcarbonyl)-3-pyrrolidinyl]methyl}-4-[2-fluoro-4-(6-isoquinolinyl)phenyl]-2,4-dihydro-3H-1,2,4-triazol-3-one, 5-{[(3S)-1-(cyclopropylcarbonyl)-3-pyrrolidinyl]methyl}-4-[2-fluoro-4-(7-isoquinolinyl)phenyl]-2,4-dihydro-3H-1,2,4-triazol-3-one, 5-{[(3S)-1-(cyclopropylcarbonyl)-3-pyrrolidinyl]methyl}-4-[4-(2,3-dihydro-1H-inden-5-yl)phenyl]-2,4-dihydro-3H-1,2,4-triazol-3-one, 5-{[(3S)-1-(cyclopropylcarbonyl)-3-pyrrolidinyl]methyl}-4-[2-fluoro-4-(2-methyl-7-quinolinyl)phenyl]-2,4-dihydro-3H-1,2,4-triazol-3-one, 5-{[(3S)-1-(cyclopropylcarbonyl)-3-pyrrolidinyl]methyl}-4-[4′-(dimethylamino)-3-fluoro-3′-methyl-4-biphenylyl]-2,4-dihydro-3H-1,2,4-triazol-3-one, 5-{[(3S)-1-(cyclopropylcarbonyl)-3-pyrrolidinyl]methyl}-4-[2-fluoro-4-(1-methyl-2,3-dihydro-1H-indol-5-yl)phenyl]-2,4-dihydro-3H-1,2,4-triazol-3-one, 5-{[(3S)-1-(cyclopropylcarbonyl)-3-pyrrolidinyl]methyl}-4-[2-fluoro-4-(3-quinolinyl)phenyl]-2,4-dihydro-3H-1,2,4-triazol-3-one, 5-{[(3S)-1-(cyclopropylcarbonyl)-3-pyrrolidinyl]methyl}-4-(3′,4′-dichloro-3-fluoro-4-biphenylyl)-2,4-dihydro-3H-1,2,4-triazol-3-one, 5-{[(3S)-1-(cyclopropylcarbonyl)-3-pyrrolidinyl]methyl}-4-[4′-(dimethylamino)-3-fluoro-2′-methyl-4-biphenylyl]-2,4-dihydro-3H-1,2,4-triazol-3-one, 4-(4′-chloro-3,3′-difluoro-4-biphenylyl)-5-{[(3S)-1-(cyclopropylcarbonyl)-3-pyrrolidinyl]methyl}-2,4-dihydro-3H-1,2,4-triazol-3-one, 4-(4′-chloro-3-fluoro-3′-methyl-4-biphenylyl)-5-{[(3S)-1-(cyclopropylcarbonyl)-3-pyrrolidinyl]methyl}-2,4-dihydro-3H-1,2,4-triazol-3-one, 4-[4′-chloro-3-fluoro-3′-(methyloxy)-4-biphenylyl]-5-{[(3S)-1-(cyclopropylcarbonyl)-3-pyrrolidinyl]methyl}-2,4-dihydro-3H-1,2,4-triazol-3-one, 5-{[(3S)-1-(cyclopropylcarbonyl)-3-pyrrolidinyl]methyl}-4-(2′,4′-dichloro-3-fluoro-4-biphenylyl)-2,4-dihydro-3H-1,2,4-triazol-3-one, 4-(4′-chloro-2′,3-difluoro-4-biphenylyl)-5-{[(3S)-1-(cyclopropylcarbonyl)-3-pyrrolidinyl]methyl}-2,4-dihydro-3H-1,2,4-triazol-3-one, 4-(4′-chloro-3-fluoro-2′-methyl-4-biphenylyl)-5-{[(3S)-1-(cyclopropylcarbonyl)-3-pyrrolidinyl]methyl}-2,4-dihydro-3H-1,2,4-triazol-3-one, 5-{[(3S)-1-(cyclopropylcarbonyl)-3-pyrrolidinyl]methyl}-4-[2-fluoro-4-(7-quinazolinyl)phenyl]-2,4-dihydro-3H-1,2,4-triazol-3-one, 4-(4′-chloro-3-fluoro-4-biphenylyl)-5-{[(3S)-1-(cyclopropylcarbonyl)-3-pyrrolidinyl]methyl}-2,4-dihydro-3H-1,2,4-triazol-3-one, 5-{[(3S)-1-(cyclopropylcarbonyl)-3-pyrrolidinyl]methyl}-4-[4-(2,3-dihydro-1H-inden-5-yl)-2-fluorophenyl]-2,4-dihydro-3H-1,2,4-triazol-3-one, 5-{[(3S)-1-(cyclopropylcarbonyl)-3-pyrrolidinyl]methyl}-4-[2-fluoro-4-(1-oxo-2,3-dihydro-1H-inden-5-yl)phenyl]-2,4-dihydro-3H-1,2,4-triazol-3-one, 5-{[(3S)-1-(cyclopropylcarbonyl)-3-pyrrolidinyl]methyl}-4-[4′-(4-morpholinyl)-4-biphenylyl]-2,4-dihydro-3H-1,2,4-triazol-3-one, 5-{[(3S)-1-(cyclopropylcarbonyl)-3-pyrrolidinyl]methyl}-4-[4′-(1H-pyrrol-1-yl)-4-biphenylyl]-2,4-dihydro-3H-1,2,4-triazol-3-one, 5-{[(3S)-1-(cyclopropylcarbonyl)-3-pyrrolidinyl]methyl}-4-[4′-(1-pyrrolidinyl)-4-biphenylyl]-2,4-dihydro-3H-1,2,4-triazol-3-one, 5-{[(3S)-1-(cyclopropylcarbonyl)-3-pyrrolidinyl]methyl}-4-[4-(7-quinolinyl)phenyl]-2,4-dihydro-3H-1,2,4-triazol-3-one, 5-{[(3S)-1-(cyclopropylcarbonyl)-3-pyrrolidinyl]methyl}-4-(2′,3,4′-trifluoro-4-biphenylyl)-2,4-dihydro-3H-1,2,4-triazol-3-one, 5-{[(3S)-1-(cyclopropylcarbonyl)-3-pyrrolidinyl]methyl}-4-[2′,3-difluoro-4′-(methyloxy)-4-biphenylyl]-2,4-dihydro-3H-1,2,4-triazol-3-one, 5-{[(3S)-1-(cyclopropylcarbonyl)-3-pyrrolidinyl]methyl}-4-[2-fluoro-4-(4-quinolinyl)phenyl]-2,4-dihydro-3H-1,2,4-triazol-3-one, N-[4′-(3-{[(3S)-1-(cyclopropylcarbonyl)-3-pyrrolidinyl]methyl}-5-oxo-1,5-dihydro-4H-1,2,4-triazol-4-yl)-3′-fluoro-4-biphenylyl]acetamide, 4-[4-(1-benzofuran-5-yl)phenyl]-5-{[(3R)-1-(cyclopropylcarbonyl)-3-pyrrolidinyl]methyl}-2,4-dihydro-3H-1,2,4-triazol-3-one, 4′-(3-{[(3S)-1-(cyclopropylcarbonyl)-3-pyrrolidinyl]methyl}-5-oxo-1,5-dihydro-4H-1,2,4-triazol-4-yl)-3′-fluoro-4-biphenylcarboxylic acid, 4′-(3-{[(3S)-1-(cyclopropylcarbonyl)-3-pyrrolidinyl]methyl}-5-oxo-1,5-dihydro-4H-1,2,4-triazol-4-yl)-3′-fluoro-3-biphenylcarboxylic acid, 5-{[1-(cyclopropylcarbonyl)-3-azetidinyl]methyl}-4-[4-(7-quinolinyl)phenyl]-2,4-dihydro-3H-1,2,4-triazol-3-one, 5-{[1-(cyclopropylcarbonyl)-3-azetidinyl]methyl}-4-[2-fluoro-4-(7-quinolinyl)phenyl]-2,4-dihydro-3H-1,2,4-triazol-3-one, 4-[2-fluoro-4-(7-quinolinyl)phenyl]-5-[(1-propanoyl-3-azetidinyl)methyl]-2,4-dihydro-3H-1,2,4-triazol-3-one, 5-[(1-propanoyl-3-azetidinyl)methyl]-4-[4-(7-quinolinyl)phenyl]-2,4-dihydro-3H-1,2,4-triazol-3-one, 3-({4-[2-fluoro-4-(7-quinolinyl)phenyl]-5-oxo-4,5-dihydro-1H-1,2,4-triazol-3-yl}methyl)-N,N-dimethyl-1-azetidinecarboxamide, 4-[2-fluoro-4-(7-quinolinyl)phenyl]-5-({1-[(1-methylcyclopropyl)carbonyl]-3-azetidinyl}methyl)-2,4-dihydro-3H-1,2,4-triazol-3-one, 4-[5-chloro-2-fluoro-4-(7-quinolinyl)phenyl]-5-{[(3S)-1-(cyclopropylcarbonyl)-3-pyrrolidinyl]methyl}-2,4-dihydro-3H-1,2,4-triazol-3-one, 4-[4-(1-benzofuran-5-yl)-5-chloro-2-fluorophenyl]-5-{[(3S)-1-(cyclopropylcarbonyl)-3-pyrrolidinyl]methyl}-2,4-dihydro-3H-1,2,4-triazol-3-one, 5-{[(3S)-1-(cyclopropylcarbonyl)-3-pyrrolidinyl]methyl}-4-[2-fluoro-5-methyl-4-(7-quinolinyl)phenyl]-2,4-dihydro-3H-1,2,4-triazol-3-one, 4-[4-(1-benzofuran-5-yl)-2-fluoro-5-methylphenyl]-5-{[(3S)-1-(cyclopropylcarbonyl)-3-pyrrolidinyl]methyl}-2,4-dihydro-3H-1,2,4-triazol-3-one, 4-[4-(1-benzofuran-5-yl)-2-chloro-6-fluorophenyl]-5-{[(3S)-1-(cyclopropylcarbonyl)-3-pyrrolidinyl]methyl}-2,4-dihydro-3H-1,2,4-triazol-3-one, 4-[4-(1-benzofuran-5-yl)-3-hydroxyphenyl]-5-{[(3S)-1-(cyclopropylcarbonyl)-3-pyrrolidinyl]methyl}-2,4-dihydro-3H-1,2,4-triazol-3-one, 6-[4-(3-{[(3S)-1-(cyclopropylcarbonyl)-3-pyrrolidinyl]methyl}-5-oxo-1,5-dihydro-4H-1,2,4-triazol-4-yl)-3-fluorophenyl]-4(1H)-quinazolinone, 7-[4-(3-{[(3S)-1-(cyclopropylcarbonyl)-3-pyrrolidinyl]methyl}-5-oxo-1,5-dihydro-4H-1,2,4-triazol-4-yl)-3-fluorophenyl]-4(1H)-quinazolinone, 4-(4′-acetyl-3-fluoro-4-biphenylyl)-5-{[(3S)-1-(cyclopropylcarbonyl)-3-pyrrolidinyl]methyl}-2,4-dihydro-3H-1,2,4-triazol-3-one, N-[4′-(3-{[(3S)-1-(cyclopropylcarbonyl)-3-pyrrolidinyl]methyl}-5-oxo-1,5-dihydro-4H-1,2,4-triazol-4-yl)-3′-fluoro-3-biphenylyl]acetamide, 5-{[(3S)-1-(cyclopropylcarbonyl)-3-pyrrolidinyl]methyl}-4-[3-fluoro-4′-(1-pyrrolidinyl)-4-biphenylyl]-2,4-dihydro-3H-1,2,4-triazol-3-one, 5-{[(3S)-1-(cyclopropylcarbonyl)-3-pyrrolidinyl]methyl}-4-[4′-(2-methyl-1,3-thiazol-4-yl)-4-biphenylyl]-2,4-dihydro-3H-1,2,4-triazol-3-one, 5-{[(3S)-1-(cyclopropylcarbonyl)-3-pyrrolidinyl]methyl}-4-[4′-(5-methyl-1,3,4-oxadiazol-2-yl)-4-biphenylyl]-2,4-dihydro-3H-1,2,4-triazol-3-one, 5-{[(3S)-1-(cyclopropylcarbonyl)-3-pyrrolidinyl]methyl}-4-[2-fluoro-4-(3-oxo-2,3-dihydro-1H-inden-5-yl)phenyl]-2,4-dihydro-3H-1,2,4-triazol-3-one, 5-{[(3S)-1-(cyclopropylcarbonyl)-3-pyrrolidinyl]methyl}-4-[4-(2,3-dihydro-1H-indol-6-yl)-2-fluorophenyl]-2,4-dihydro-3H-1,2,4-triazol-3-one, 5-{[(3S)-1-(cyclopropylcarbonyl)-3-pyrrolidinyl]methyl}-4-[3-fluoro-4′-(2-oxo-1-pyrrolidinyl)-4-biphenylyl]-2,4-dihydro-3H-1,2,4-triazol-3-one, 5-{[(3S)-1-(cyclopropylcarbonyl)-3-pyrrolidinyl]methyl}-4-[2-fluoro-4-(1,2,3,4-tetrahydro-7-quinolinyl)phenyl]-2,4-dihydro-3H-1,2,4-triazol-3-one, 5-{[(3S)-1-acetyl-3-pyrrolidinyl]methyl}-4-[4-(7-quinolinyl)phenyl]-2,4-dihydro-3H-1,2,4-triazol-3-one, 5-{[(3S)-1-propanoyl-3-pyrrolidinyl]methyl}-4-[4-(7-quinolinyl)phenyl]-2,4-dihydro-3H-1,2,4-triazol-3-one, (3S)—N,N-dimethyl-3-({5-oxo-4-[4-(7-quinolinyl)phenyl]-4,5-dihydro-1H-1,2,4-triazol-3-yl}methyl)-1-pyrrolidinecarboxamide, 5-{[(3S)-1-(2-methylpropanoyl)-3-pyrrolidinyl]methyl}-4-[4-(7-quinolinyl)phenyl]-2,4-dihydro-3H-1,2,4-triazol-3-one, 5-{[(3S)-1-(2,2-dimethylpropanoyl)-3-pyrrolidinyl]methyl}-4-[4-(7-quinolinyl)phenyl]-2,4-dihydro-3H-1,2,4-triazol-3-one, 5-({(3S)-1-[(1-methylcyclopropyl)carbonyl]-3-pyrrolidinyl}methyl)-4-[4-(7-quinolinyl)phenyl]-2,4-dihydro-3H-1,2,4-triazol-3-one, (3S)-3-({4-[3-fluoro-4′-(methyloxy)-4-biphenylyl]-5-oxo-4,5-dihydro-1H-1,2,4-triazol-3-yl}methyl)-N,N-dimethyl-1-pyrrolidinecarboxamide, 4-[3-fluoro-4′-(methyloxy)-4-biphenylyl]-5-{[(3S)-1-propanoyl-3-pyrrolidinyl]methyl}-2,4-dihydro-3H-1,2,4-triazol-3-one, 5-{[(3S)-1-(2,2-dimethylpropanoyl)-3-pyrrolidinyl]methyl}-4-[3-fluoro-4′-(methyloxy)-4-biphenylyl]-2,4-dihydro-3H-1,2,4-triazol-3-one, (3S)-3-({4 2-fluoro-4 7-quinolinyl)phenyl]-5-oxo-4,5-dihydro-1H-1,2,4-triazol-3-yl}methyl)-N,N-dimethyl-1-pyrrolidinecarboxamide, 4-[3-fluoro-4′-(methyloxy)-4-biphenylyl]-5-({(3S)-1-[(1-methylcyclopropyl)carbonyl]-3-pyrrolidinyl}methyl)-2,4-dihydro-3H-1,2,4-triazol-3-one, 4-[2-fluoro-4-(7-quinolinyl)phenyl]-5-{[(3S)-1-propanoyl-3-pyrrolidinyl]methyl}-2,4-dihydro-3H-1,2,4-triazol-3-one, 5-{[(3S)-1-(2,2-dimethylpropanoyl)-3-pyrrolidinyl]methy}-4-[2-fluoro-4-(7-quinolinyl)phenyl]-2,4-dihydro-3H-1,2,4-triazol-3-one, 4-[2-fluoro-4-(7-quinolinyl)phenyl]-5-({(3S)-1-[(1-methylcyclopropyl)carbonyl]-3-pyrrolidinyl}methyl)-2,4-dihydro-3H-1,2,4-triazol-3-one, (3S)—N-ethyl-3-({4-[2-fluoro-4-(7-quinolinyl)phenyl]-5-oxo-4,5-dihydro-1H-1,2,4-triazol-3-yl}methyl)-1-pyrrolidinecarboxamide, 5-{[(3S)-1-(4-morpholinylcarbonyl)-3-pyrrolidinyl]methyl}-4-[4-(7-quinolinyl)phenyl]-2,4-dihydro-3H-1,2,4-triazol-3-one, 4-[3-fluoro-4′-(methyloxy)-4-biphenylyl]-5-{[(3S)-1-(2-methylpropanoyl)-3-pyrrolidinyl]methyl}-2,4-dihydro-3H-1,2,4-triazol-3-one, 4-[2-fluoro-4-(7-quinolinyl)phenyl]-5-{[(3S)-1-(2-methylpropanoyl)-3-pyrrolidinyl]methyl}-2,4-dihydro-3H-1,2,4-triazol-3-one, 4-[3-fluoro-3′-(methyloxy)-4-biphenylyl]-5-{[(3S)-1-propanoyl-3-pyrrolidinyl]methyl}-2,4-dihydro-3H-1,2,4-triazol-3-one, 5-{[(3S)-1-(cyclopropylcarbonyl)-3-pyrrolidinyl]methyl}-4-[3-fluoro-3′-(methyloxy)-4-biphenylyl]-2,4-dihydro-3H-1,2,4-triazol-3-one, 5-{[(3S)-1-(cyclopropylcarbonyl)-3-pyrrolidinyl]methyl}-4-(3-fluoro-3′-hydroxy-4-biphenylyl)-2,4-dihydro-3H-1,2,4-triazol-3-one, 5-{[(3S)-1-(cyclopropylcarbonyl)-3-pyrrolidinyl]methyl}-4-(3-fluoro-4′-hydroxy-4-biphenylyl)-2,4-dihydro-3H-1,2,4-triazol-3-one, 5-{[(3S)-1-(cyclopropylcarbonyl)-3-pyrrolidinyl]methyl}-4-[2-fluoro-4-(6-fluoro-2-naphthalenyl)phenyl]-2,4-dihydro-3H-1,2,4-triazol-3-one, 5-{[(3S)-1-(cyclopropylcarbonyl)-3-pyrrolidinyl]methyl}-4-[2-fluoro-4-(8-fluoro-2-naphthalenyl)phenyl]-2,4-dihydro-3H-1,2,4-triazol-3-one, and pharmaceutically acceptable salts thereof.

In some embodiments, the compound is (S)-3-((1-(cyclopropanecarbonyl)pyrrolidin-3-yl)methyl)-4-(2-fluoro-4-(3-methylquinolin-7-yl)phenyl)-1H-1,2,4-triazol-5(4H)-one; (S)-4-(4-(3-chloroquinolin-7-yl)-2-fluorophenyl)-3-((1-(cyclopropanecarbonyl)pyrrolidin-3-yl)methyl)-1H-1,2,4-triazol-5(4H)-one; (S)-3-((1-(cyclopropanecarbonyl)pyrrolidin-3-yl)methyl)-4-(2-fluoro-4-(3-fluoroquinolin-7-yl)phenyl)-1H-1,2,4-triazol-5(4H)-one; (S)-4-(2-fluoro-4-(3-fluoroquinolin-7-yl)phenyl)-3-((1-propionylpyrrolidin-3-yl)methyl)-1H-1,2,4-triazol-5(4H)-one; (S)-4-(2-fluoro-4-(3-methylquinolin-7-yl)phenyl)-3-((1-propionylpyrrolidin-3-yl)methyl)-1H-1,2,4-triazol-5(4H)-one; (S)-4-(4-(3-chloroquinolin-7-yl)-2-fluorophenyl)-3-((1-propionylpyrrolidin-3-yl)methyl)-1H-1,2,4-triazol-5(4H)-one; (S)-4-(2-fluoro-4-(3-methylquinolin-7-yl)phenyl)-1-methyl-3-((1-propionylpyrrolidin-3-yl)methyl)-1H-1,2,4-triazol-5(4H)-one; (S)-4-(4-(3-chloroquinolin-7-yl)-2-fluorophenyl)-1-methyl-3-((1-propionylpyrrolidin-3-yl)methyl)-1H-1,2,4-triazol-5(4H)-one; (S)-4-(4-(3-chloroquinolin-7-yl)-2-fluorophenyl)-3-((1-(cyclopropanecarbonyl)pyrrolidin-3-yl)methyl)-1-methyl-1H-1,2,4-triazol-5(4H)-one; and (S)-4-(2-fluoro-4-(3-fluoroquinolin-7-yl)phenyl)-1-methyl-3-((1-propionylpyrrolidin-3-yl)methyl)-1H-1,2,4-triazol-5(4H)-one, or a pharmaceutically acceptable salt thereof.

In some embodiments, the compound is one of the following:

| | Compound |
|---|---|
| 916 | 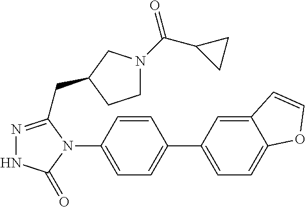 [SMILES: O=C(C1CC1)N1CC[C@@H](Cc2n[nH]c(=O)n2-c2ccc(-c3ccc4occc4c3)cc2)C1] |
| 917 | 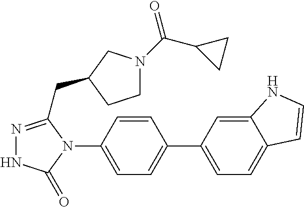 [SMILES: O=C(C1CC1)N1CC[C@@H](Cc2n[nH]c(=O)n2-c2ccc(-c3ccc4cc[nH]c4c3)cc2)C1] |
| 918 | 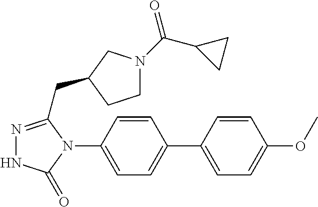 [SMILES: COc1ccc(-c2ccc(-n3c(C[C@@H]4CCN(C(=O)C5CC5)C4)n[nH]c3=O)cc2)cc1] |
| 919 | 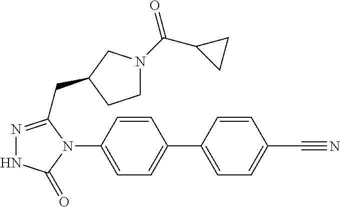 [SMILES: N#Cc1ccc(-c2ccc(-n3c(C[C@@H]4CCN(C(=O)C5CC5)C4)n[nH]c3=O)cc2)cc1] |
| 920 | 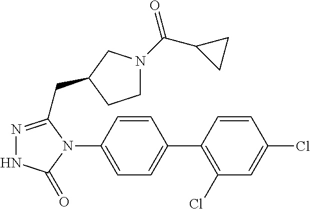 [SMILES: O=C(C1CC1)N1CC[C@@H](Cc2n[nH]c(=O)n2-c2ccc(-c3ccc(Cl)cc3Cl)cc2)C1] |
| 921 | 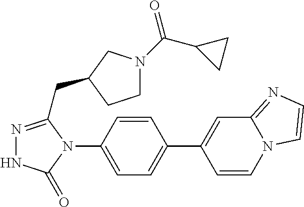 [SMILES: O=C(C1CC1)N1CC[C@@H](Cc2n[nH]c(=O)n2-c2ccc(-c3ccn4ccnc4c3)cc2)C1] |
| 922 | 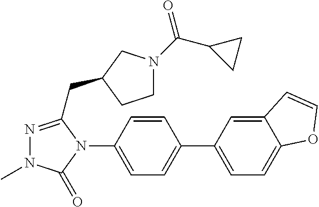 [SMILES: Cn1nc(C[C@@H]2CCN(C(=O)C3CC3)C2)n(-c2ccc(-c3ccc4occc4c3)cc2)c1=O] |
| 923 | 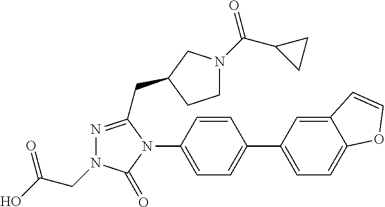 [SMILES: O=C(O)Cn1nc(C[C@@H]2CCN(C(=O)C3CC3)C2)n(-c2ccc(-c3ccc4occc4c3)cc2)c1=O] |
| 924 | 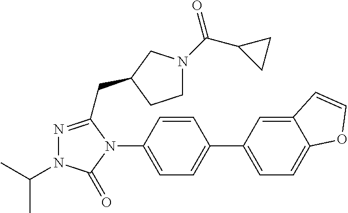 [SMILES: CC(C)n1nc(C[C@@H]2CCN(C(=O)C3CC3)C2)n(-c2ccc(-c3ccc4occc4c3)cc2)c1=O] |
| 925 | 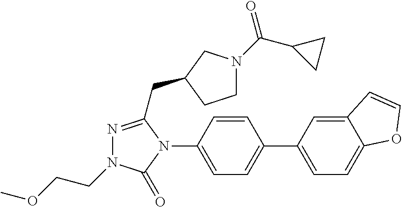 [SMILES: COCCn1nc(C[C@@H]2CCN(C(=O)C3CC3)C2)n(-c2ccc(-c3ccc4occc4c3)cc2)c1=O] |
| 926 | 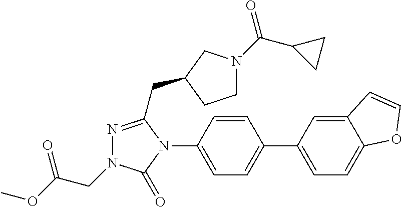 [SMILES: COC(=O)Cn1nc(C[C@@H]2CCN(C(=O)C3CC3)C2)n(-c2ccc(-c3ccc4occc4c3)cc2)c1=O] |
| 927 | 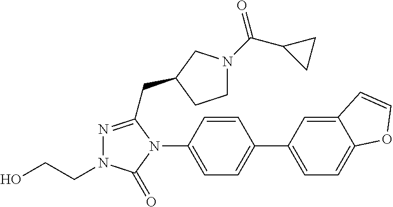 [SMILES: O=C(C1CC1)N1CC[C@@H](Cc2nn(CCO)c(=O)n2-c2ccc(-c3ccc4occc4c3)cc2)C1] |
| 928 | 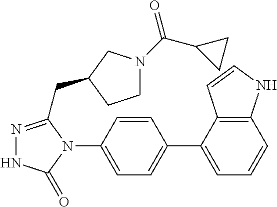 [SMILES: O=C(C1CC1)N1CC[C@@H](Cc2n[nH]c(=O)n2-c2ccc(-c3cccc4[nH]ccc34)cc2)C1] |
| 929 | 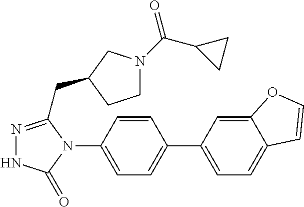 [SMILES: O=C(C1CC1)N1CC[C@@H](Cc2n[nH]c(=O)n2-c2ccc(-c3ccc4ccoc4c3)cc2)C1] |
| 930 | 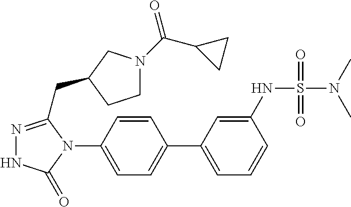 [SMILES: CN(C)S(=O)(=O)Nc1cccc(-c2ccc(-n3c(C[C@@H]4CCN(C(=O)C5CC5)C4)n[nH]c3=O)cc2)c1] |
| 931 | 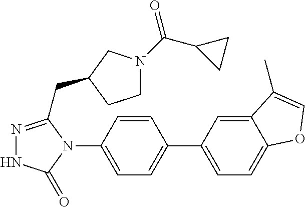 [SMILES: Cc1coc2ccc(-c3ccc(-n4c(C[C@@H]5CCN(C(=O)C6CC6)C5)n[nH]c4=O)cc3)cc12] |
| 932 | 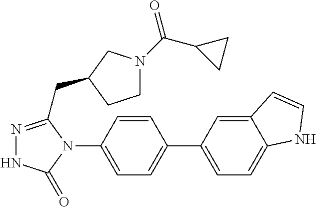 [SMILES: O=C(C1CC1)N1CC[C@@H](Cc2n[nH]c(=O)n2-c2ccc(-c3ccc4[nH]ccc4c3)cc2)C1] |
| 933 | 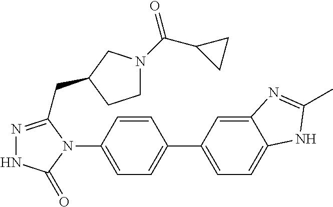 [SMILES: Cc1nc2cc(-c3ccc(-n4c(C[C@@H]5CCN(C(=O)C6CC6)C5)n[nH]c4=O)cc3)ccc2[nH]1] |
| 934 | 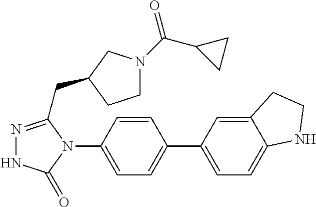 [SMILES: O=C(C1CC1)N1CC[C@@H](Cc2n[nH]c(=O)n2-c2ccc(-c3ccc4c(c3)CCN4)cc2)C1] |
| 935 | 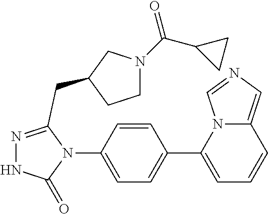 [SMILES: O=C(C1CC1)N1CC[C@@H](Cc2n[nH]c(=O)n2-c2ccc(-c3cccc4cncn34)cc2)C1] |
| 936 | 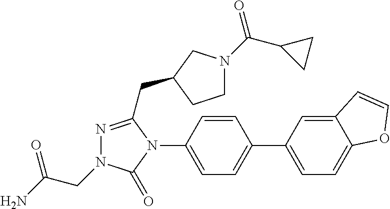 [SMILES: NC(=O)Cn1nc(C[C@@H]2CCN(C(=O)C3CC3)C2)n(-c2ccc(-c3ccc4occc4c3)cc2)c1=O] |
| 937 | 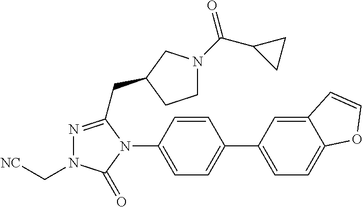 [SMILES: N#CCn1nc(C[C@@H]2CCN(C(=O)C3CC3)C2)n(-c2ccc(-c3ccc4occc4c3)cc2)c1=O] |
| 938 | 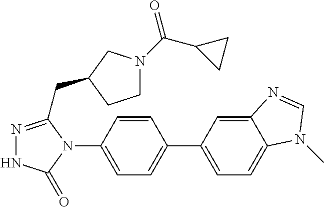 [SMILES: Cn1cnc2cc(-c3ccc(-n4c(C[C@@H]5CCN(C(=O)C6CC6)C5)n[nH]c4=O)cc3)ccc21] |
| 939 | 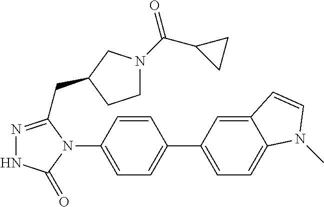 [SMILES: Cn1ccc2cc(-c3ccc(-n4c(C[C@@H]5CCN(C(=O)C6CC6)C5)n[nH]c4=O)cc3)ccc21] |
| 940 | 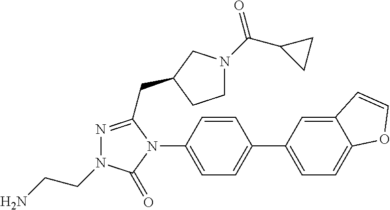 [SMILES: NCCn1nc(C[C@@H]2CCN(C(=O)C3CC3)C2)n(-c2ccc(-c3ccc4occc4c3)cc2)c1=O] |
| 941 | 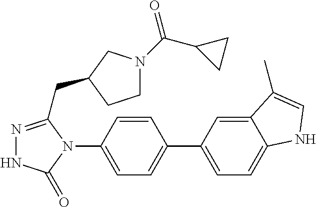 [SMILES: Cc1c[nH]c2ccc(-c3ccc(-n4c(C[C@@H]5CCN(C(=O)C6CC6)C5)n[nH]c4=O)cc3)cc12] |
| 942 | 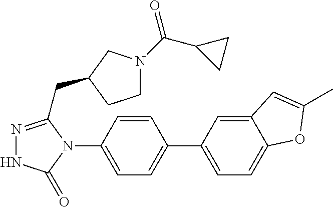 [SMILES: Cc1cc2cc(-c3ccc(-n4c(C[C@@H]5CCN(C(=O)C6CC6)C5)n[nH]c4=O)cc3)ccc2o1] |
| 943 | 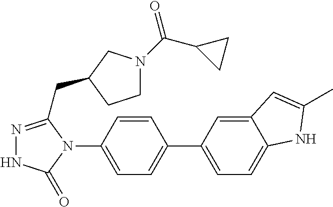 [SMILES: Cc1cc2cc(-c3ccc(-n4c(C[C@@H]5CCN(C(=O)C6CC6)C5)n[nH]c4=O)cc3)ccc2[nH]1] |
| 944 | 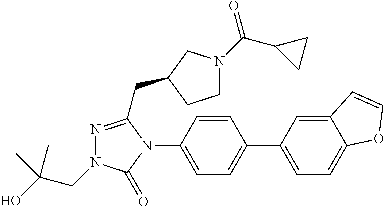 [SMILES: CC(C)(O)Cn1nc(C[C@@H]2CCN(C(=O)C3CC3)C2)n(-c2ccc(-c3ccc4occc4c3)cc2)c1=O] |
| 945 | 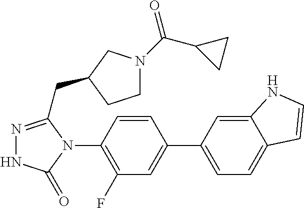 [SMILES: O=C(C1CC1)N1CC[C@@H](Cc2n[nH]c(=O)n2-c2ccc(-c3ccc4cc[nH]c4c3)cc2F)C1] |
| 946 | 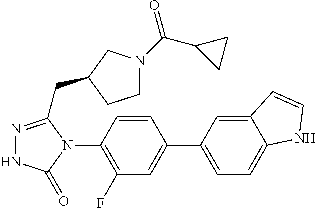 [SMILES: O=C(C1CC1)N1CC[C@@H](Cc2n[nH]c(=O)n2-c2ccc(-c3ccc4[nH]ccc4c3)cc2F)C1] |
| 947 | 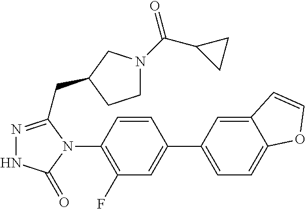 [SMILES: O=C(C1CC1)N1CC[C@@H](Cc2n[nH]c(=O)n2-c2ccc(-c3ccc4occc4c3)cc2F)C1] |
| 948 | 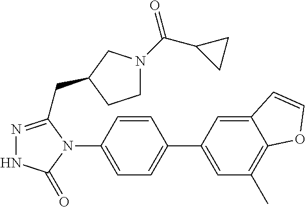 [SMILES: Cc1cc(-c2ccc(-n3c(C[C@@H]4CCN(C(=O)C5CC5)C4)n[nH]c3=O)cc2)cc2ccoc12] |
| 949 | 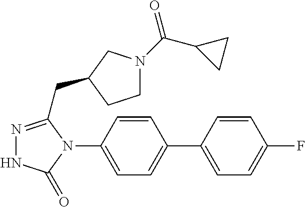 [SMILES: O=C(C1CC1)N1CC[C@@H](Cc2n[nH]c(=O)n2-c2ccc(-c3ccc(F)cc3)cc2)C1] |
| 950 | 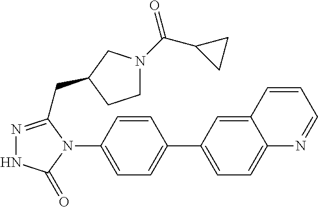 [SMILES: O=C(C1CC1)N1CC[C@@H](Cc2n[nH]c(=O)n2-c2ccc(-c3ccc4ncccc4c3)cc2)C1] |
| 951 | 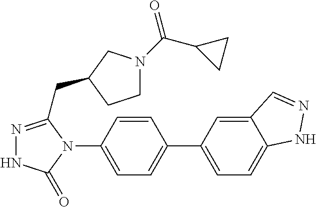 [SMILES: O=C(C1CC1)N1CC[C@@H](Cc2n[nH]c(=O)n2-c2ccc(-c3ccc4[nH]ncc4c3)cc2)C1] |
| 952 | 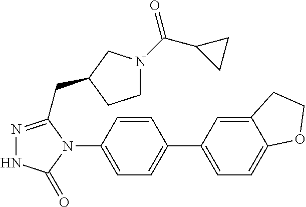 [SMILES: O=C(C1CC1)N1CC[C@@H](Cc2n[nH]c(=O)n2-c2ccc(-c3ccc4c(c3)CCO4)cc2)C1] |
| 953 | 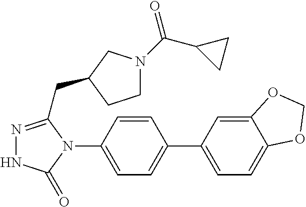 [SMILES: O=C(C1CC1)N1CC[C@@H](Cc2n[nH]c(=O)n2-c2ccc(-c3ccc4c(c3)OCO4)cc2)C1] |
| 954 | 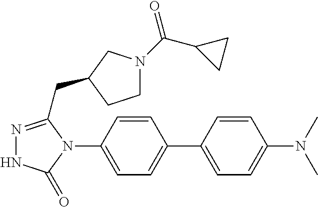 [SMILES: CN(C)c1ccc(-c2ccc(-n3c(C[C@@H]4CCN(C(=O)C5CC5)C4)n[nH]c3=O)cc2)cc1] |
| 955 | 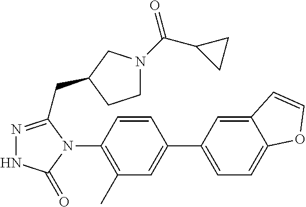 [SMILES: Cc1cc(-c2ccc3occc3c2)ccc1-n1c(C[C@@H]2CCN(C(=O)C3CC3)C2)n[nH]c1=O] |
| 956 | 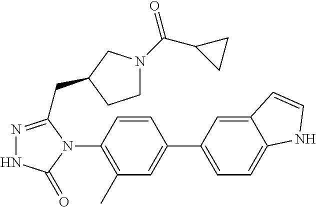 [SMILES: Cc1cc(-c2ccc3[nH]ccc3c2)ccc1-n1c(C[C@@H]2CCN(C(=O)C3CC3)C2)n[nH]c1=O] |
| 957 | 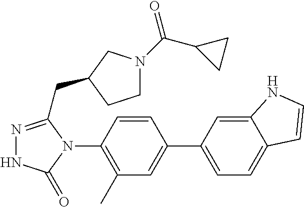 [SMILES: Cc1cc(-c2ccc3cc[nH]c3c2)ccc1-n1c(C[C@@H]2CCN(C(=O)C3CC3)C2)n[nH]c1=O] |
| 958 | 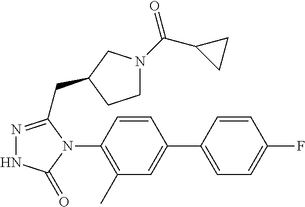 [SMILES: Cc1cc(-c2ccc(F)cc2)ccc1-n1c(C[C@@H]2CCN(C(=O)C3CC3)C2)n[nH]c1=O] |
| 959 | 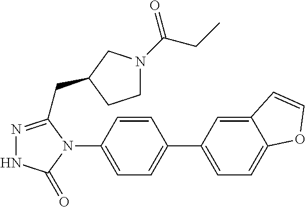 [SMILES: CCC(=O)N1CC[C@@H](Cc2n[nH]c(=O)n2-c2ccc(-c3ccc4occc4c3)cc2)C1] |
| 960 | 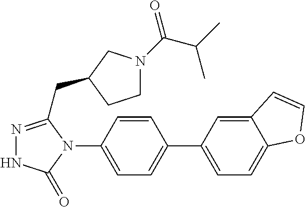 [SMILES: CC(C)C(=O)N1CC[C@@H](Cc2n[nH]c(=O)n2-c2ccc(-c3ccc4occc4c3)cc2)C1] |
| 961 | 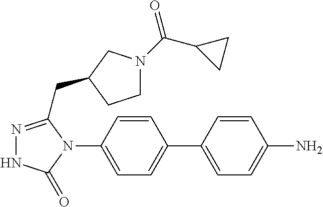 [SMILES: Nc1ccc(-c2ccc(-n3c(C[C@@H]4CCN(C(=O)C5CC5)C4)n[nH]c3=O)cc2)cc1] |
| 962 | 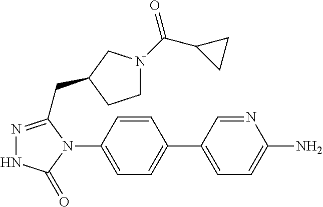 [SMILES: Nc1ccc(-c2ccc(-n3c(C[C@@H]4CCN(C(=O)C5CC5)C4)n[nH]c3=O)cc2)cn1] |
| 963 | 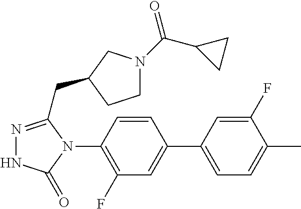 [SMILES: Cc1ccc(-c2ccc(-n3c(C[C@@H]4CCN(C(=O)C5CC5)C4)n[nH]c3=O)c(F)c2)cc1F] |
| 964 | 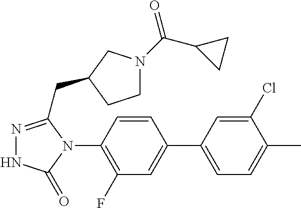 [SMILES: Cc1ccc(-c2ccc(-n3c(C[C@@H]4CCN(C(=O)C5CC5)C4)n[nH]c3=O)c(F)c2)cc1Cl] |
| 965 | 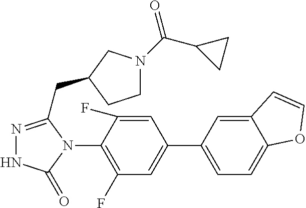 [SMILES: O=C(C1CC1)N1CC[C@@H](Cc2n[nH]c(=O)n2-c2c(F)cc(-c3ccc4occc4c3)cc2F)C1] |
| 966 | 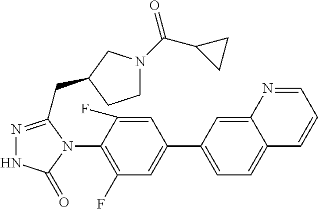 [SMILES: O=C(C1CC1)N1CC[C@@H](Cc2n[nH]c(=O)n2-c2c(F)cc(-c3ccc4cccnc4c3)cc2F)C1] |
| 967 | 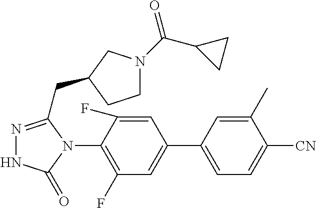 [SMILES: Cc1cc(-c2cc(F)c(-n3c(C[C@@H]4CCN(C(=O)C5CC5)C4)n[nH]c3=O)c(F)c2)ccc1C#N] |
| 968 | 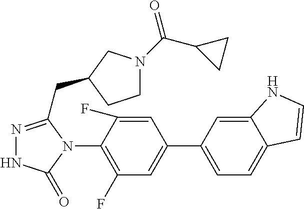 [SMILES: O=C(C1CC1)N1CC[C@@H](Cc2n[nH]c(=O)n2-c2c(F)cc(-c3ccc4cc[nH]c4c3)cc2F)C1] |
| 969 | 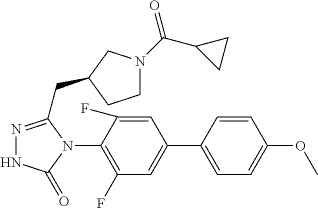 [SMILES: COc1ccc(-c2cc(F)c(-n3c(C[C@@H]4CCN(C(=O)C5CC5)C4)n[nH]c3=O)c(F)c2)cc1] |
| 970 | 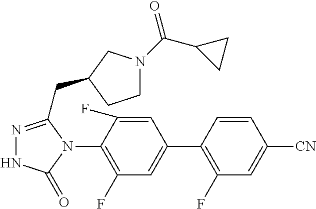 [SMILES: N#Cc1ccc(-c2cc(F)c(-n3c(C[C@@H]4CCN(C(=O)C5CC5)C4)n[nH]c3=O)c(F)c2)c(F)c1] |
| 971 | 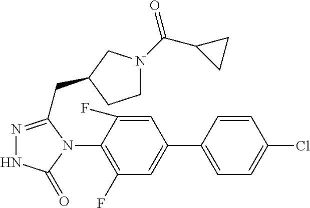 [SMILES: O=C(C1CC1)N1CC[C@@H](Cc2n[nH]c(=O)n2-c2c(F)cc(-c3ccc(Cl)cc3)cc2F)C1] |
| 972 | 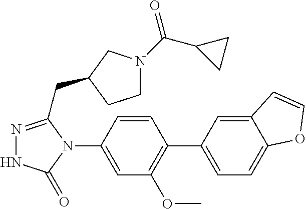 [SMILES: COc1cc(-n2c(C[C@@H]3CCN(C(=O)C4CC4)C3)n[nH]c2=O)ccc1-c1ccc2occc2c1] |
| 973 | 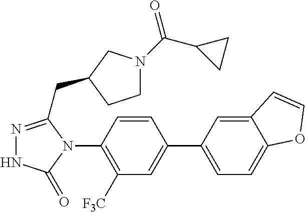 [SMILES: O=C(C1CC1)N1CC[C@@H](Cc2n[nH]c(=O)n2-c2ccc(-c3ccc4occc4c3)cc2C(F)(F)F)C1] |
| 974 | 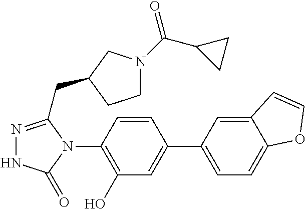 [SMILES: O=C(C1CC1)N1CC[C@@H](Cc2n[nH]c(=O)n2-c2ccc(-c3ccc4occc4c3)cc2O)C1] |
| 975 | 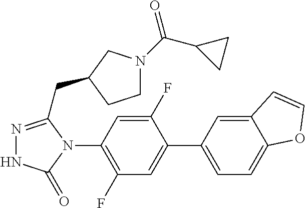 [SMILES: O=C(C1CC1)N1CC[C@@H](Cc2n[nH]c(=O)n2-c2cc(F)c(-c3ccc4occc4c3)cc2F)C1] |
| 976 | 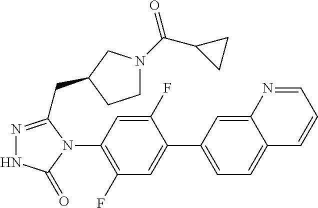 [SMILES: O=C(C1CC1)N1CC[C@@H](Cc2n[nH]c(=O)n2-c2cc(F)c(-c3ccc4cccnc4c3)cc2F)C1] |
| 977 | 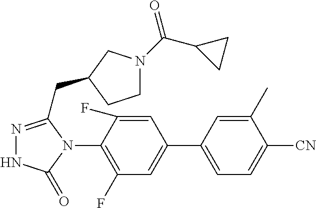 [SMILES: Cc1cc(-c2cc(F)c(-n3c(C[C@@H]4CCN(C(=O)C5CC5)C4)n[nH]c3=O)c(F)c2)ccc1C#N] |
| 978 | 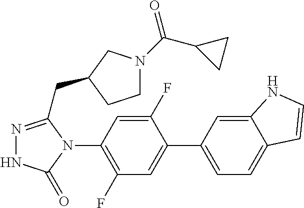 [SMILES: O=C(C1CC1)N1CC[C@@H](Cc2n[nH]c(=O)n2-c2cc(F)c(-c3ccc4cc[nH]c4c3)cc2F)C1] |
| 979 | 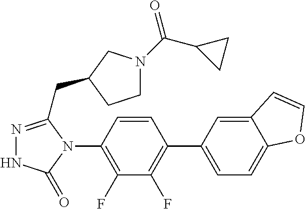 [SMILES: O=C(C1CC1)N1CC[C@@H](Cc2n[nH]c(=O)n2-c2ccc(-c3ccc4occc4c3)c(F)c2F)C1] |
| 980 | 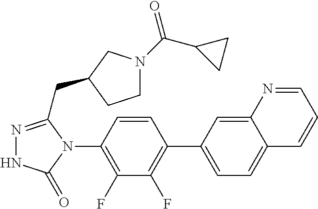 [SMILES: O=C(C1CC1)N1CC[C@@H](Cc2n[nH]c(=O)n2-c2ccc(-c3ccc4cccnc4c3)c(F)c2F)C1] |
| 981 | 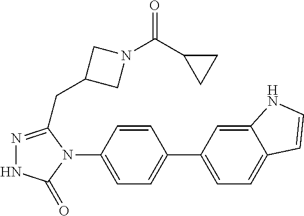 [SMILES: O=C(C1CC1)N1CC(Cc2n[nH]c(=O)n2-c2ccc(-c3ccc4cc[nH]c4c3)cc2)C1] |
| 982 | 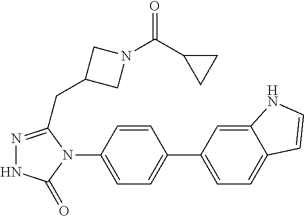 [SMILES: O=C(C1CC1)N1CC(Cc2n[nH]c(=O)n2-c2ccc(-c3ccc4cc[nH]c4c3)cc2)C1] |
| 983 | 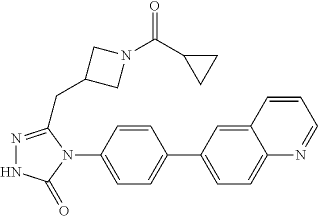 [SMILES: O=C(C1CC1)N1CC(Cc2n[nH]c(=O)n2-c2ccc(-c3ccc4ncccc4c3)cc2)C1] |
| 984 | 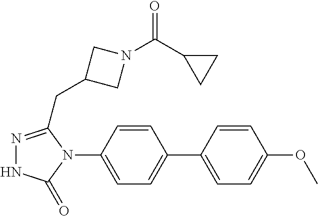 [SMILES: COc1ccc(-c2ccc(-n3c(CC4CN(C(=O)C5CC5)C4)n[nH]c3=O)cc2)cc1] |
| 985 | 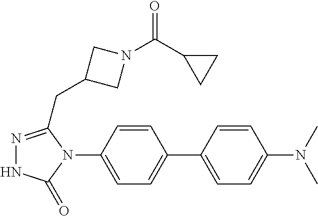 [SMILES: CN(C)c1ccc(-c2ccc(-n3c(CC4CN(C(=O)C5CC5)C4)n[nH]c3=O)cc2)cc1] |
| 986 | 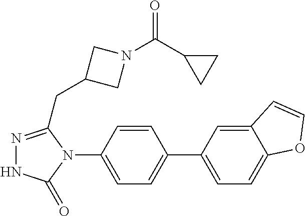 [SMILES: O=C(C1CC1)N1CC(Cc2n[nH]c(=O)n2-c2ccc(-c3ccc4occc4c3)cc2)C1] |
| 987 | 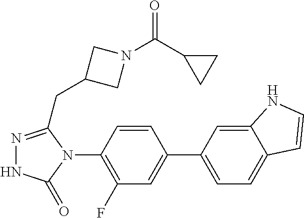 [SMILES: O=C(C1CC1)N1CC(Cc2n[nH]c(=O)n2-c2ccc(-c3ccc4cc[nH]c4c3)cc2F)C1] |
| 988 | 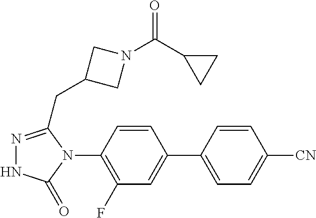 [SMILES: N#Cc1ccc(-c2ccc(-n3c(CC4CN(C(=O)C5CC5)C4)n[nH]c3=O)c(F)c2)cc1] |
| 989 | 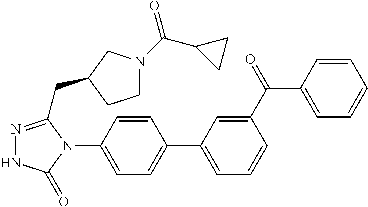 [SMILES: O=C(c1ccccc1)c1cccc(-c2ccc(-n3c(C[C@@H]4CCN(C(=O)C5CC5)C4)n[nH]c3=O)cc2)c1] |
| 990 | 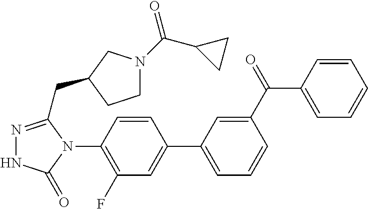 [SMILES: O=C(c1ccccc1)c1cccc(-c2ccc(-n3c(C[C@@H]4CCN(C(=O)C5CC5)C4)n[nH]c3=O)c(F)c2)c1] |
| 991 | 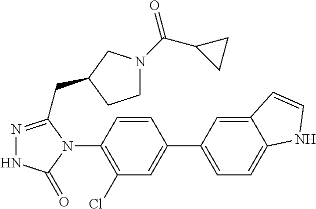 [SMILES: O=C(C1CC1)N1CC[C@@H](Cc2n[nH]c(=O)n2-c2ccc(-c3ccc4[nH]ccc4c3)cc2Cl)C1] |
| 992 | 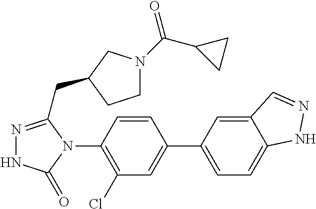 [SMILES: O=C(C1CC1)N1CC[C@@H](Cc2n[nH]c(=O)n2-c2ccc(-c3ccc4[nH]ncc4c3)cc2Cl)C1] |
| 993 | 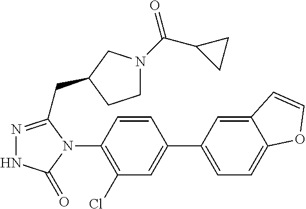 [SMILES: O=C(C1CC1)N1CC[C@@H](Cc2n[nH]c(=O)n2-c2ccc(-c3ccc4occc4c3)cc2Cl)C1] |
| 994 | 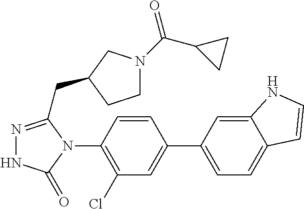 [SMILES: O=C(C1CC1)N1CC[C@@H](Cc2n[nH]c(=O)n2-c2ccc(-c3ccc4cc[nH]c4c3)cc2Cl)C1] |
| 995 | 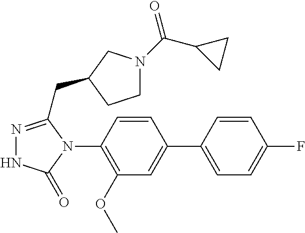 [SMILES: COc1cc(-c2ccc(F)cc2)ccc1-n1c(C[C@@H]2CCN(C(=O)C3CC3)C2)n[nH]c1=O] |
| 996 | 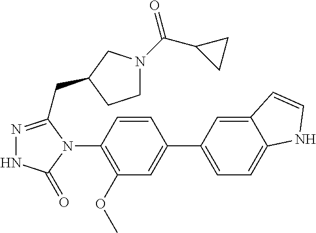 [SMILES: COc1cc(-c2ccc3[nH]ccc3c2)ccc1-n1c(C[C@@H]2CCN(C(=O)C3CC3)C2)n[nH]c1=O] |
| 997 | 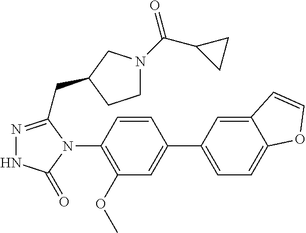 [SMILES: COc1cc(-c2ccc3occc3c2)ccc1-n1c(C[C@@H]2CCN(C(=O)C3CC3)C2)n[nH]c1=O] |
| 998 | 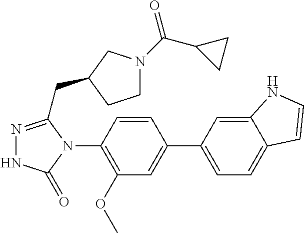 [SMILES: COc1cc(-c2ccc3cc[nH]c3c2)ccc1-n1c(C[C@@H]2CCN(C(=O)C3CC3)C2)n[nH]c1=O] |
| 999 | 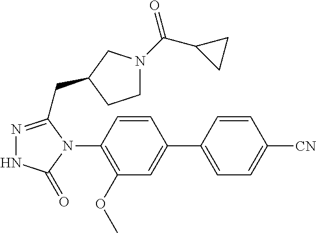 [SMILES: COc1cc(-c2ccc(C#N)cc2)ccc1-n1c(C[C@@H]2CCN(C(=O)C3CC3)C2)n[nH]c1=O] |
| 1000 | 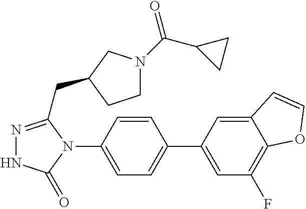 [SMILES: O=C(C1CC1)N1CC[C@@H](Cc2n[nH]c(=O)n2-c2ccc(-c3cc(F)c4occc4c3)cc2)C1] |
| 1001 | 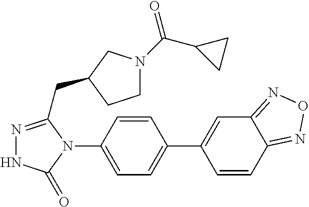 [SMILES: O=C(C1CC1)N1CC[C@@H](Cc2n[nH]c(=O)n2-c2ccc(-c3ccc4nonc4c3)cc2)C1] |
| 1002 | 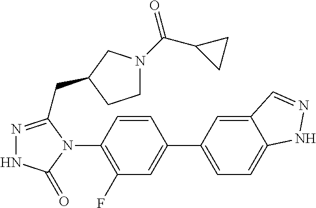 [SMILES: O=C(C1CC1)N1CC[C@@H](Cc2n[nH]c(=O)n2-c2ccc(-c3ccc4[nH]ncc4c3)cc2F)C1] |
| 1003 | 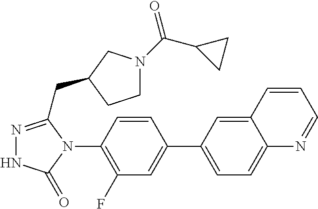 [SMILES: O=C(C1CC1)N1CC[C@@H](Cc2n[nH]c(=O)n2-c2ccc(-c3ccc4ncccc4c3)cc2F)C1] |
| 1004 | 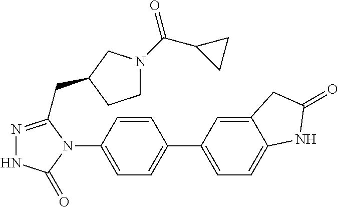 [SMILES: O=C1Cc2cc(-c3ccc(-n4c(C[C@@H]5CCN(C(=O)C6CC6)C5)n[nH]c4=O)cc3)ccc2N1] |
| 1005 | 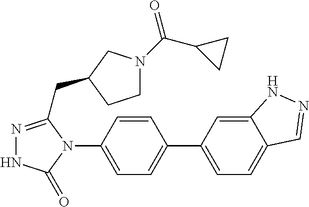 [SMILES: O=C(C1CC1)N1CC[C@@H](Cc2n[nH]c(=O)n2-c2ccc(-c3ccc4cn[nH]c4c3)cc2)C1] |
| 1006 | 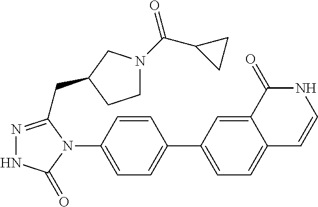 [SMILES: O=C(C1CC1)N1CC[C@@H](Cc2n[nH]c(=O)n2-c2ccc(-c3ccc4cc[nH]c(=O)c4c3)cc2)C1] |
| 1007 | 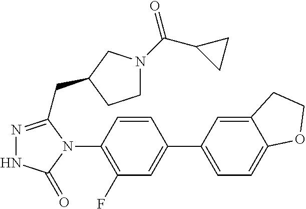 [SMILES: O=C(C1CC1)N1CC[C@@H](Cc2n[nH]c(=O)n2-c2ccc(-c3ccc4c(c3)CCO4)cc2F)C1] |
| 1008 | 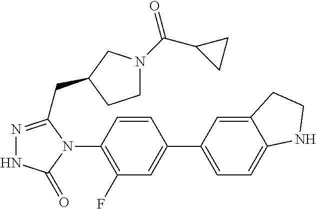 [SMILES: O=C(C1CC1)N1CC[C@@H](Cc2n[nH]c(=O)n2-c2ccc(-c3ccc4c(c3)CCN4)cc2F)C1] |
| 1009 | 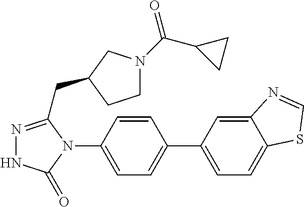 [SMILES: O=C(C1CC1)N1CC[C@@H](Cc2n[nH]c(=O)n2-c2ccc(-c3ccc4scnc4c3)cc2)C1] |
| 1010 | 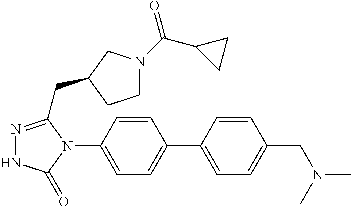 [SMILES: CN(C)Cc1ccc(-c2ccc(-n3c(C[C@@H]4CCN(C(=O)C5CC5)C4)n[nH]c3=O)cc2)cc1] |
| 1011 | 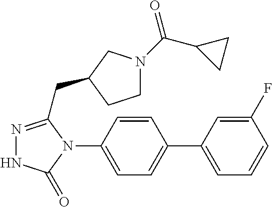 [SMILES: O=C(C1CC1)N1CC[C@@H](Cc2n[nH]c(=O)n2-c2ccc(-c3cccc(F)c3)cc2)C1] |
| 1012 | 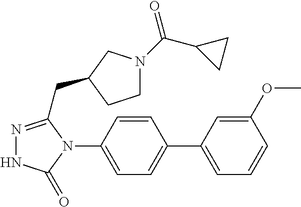 [SMILES: COc1cccc(-c2ccc(-n3c(C[C@@H]4CCN(C(=O)C5CC5)C4)n[nH]c3=O)cc2)c1] |
| 1013 | 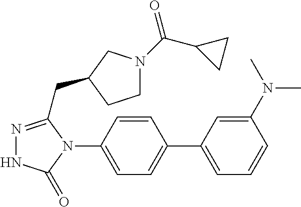 [SMILES: CN(C)c1cccc(-c2ccc(-n3c(C[C@@H]4CCN(C(=O)C5CC5)C4)n[nH]c3=O)cc2)c1] |
| 1014 | 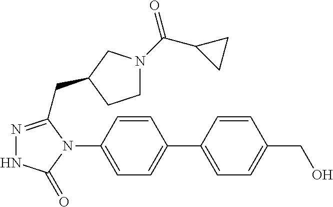 [SMILES: O=C(C1CC1)N1CC[C@@H](Cc2n[nH]c(=O)n2-c2ccc(-c3ccc(CO)cc3)cc2)C1] |
| 1015 | 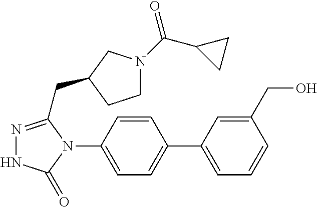 [SMILES: O=C(C1CC1)N1CC[C@@H](Cc2n[nH]c(=O)n2-c2ccc(-c3cccc(CO)c3)cc2)C1] |
| 1016 | 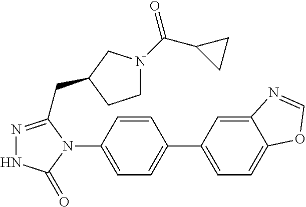 [SMILES: O=C(C1CC1)N1CC[C@@H](Cc2n[nH]c(=O)n2-c2ccc(-c3ccc4ocnc4c3)cc2)C1] |
| 1017 | 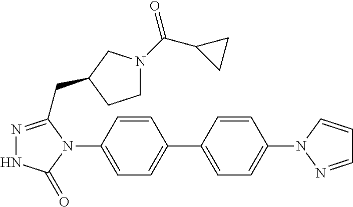 [SMILES: O=C(C1CC1)N1CC[C@@H](Cc2n[nH]c(=O)n2-c2ccc(-c3ccc(-n4cccn4)cc3)cc2)C1] |
| 1018 | 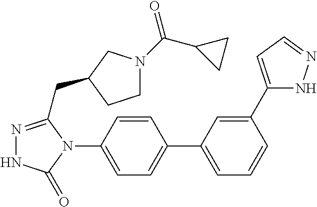 [SMILES: O=C(C1CC1)N1CC[C@@H](Cc2n[nH]c(=O)n2-c2ccc(-c3cccc(-c4ccn[nH]4)c3)cc2)C1] |
| 1019 | 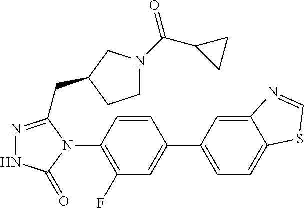 [SMILES: O=C(C1CC1)N1CC[C@@H](Cc2n[nH]c(=O)n2-c2ccc(-c3ccc4scnc4c3)cc2F)C1] |
| 1020 | 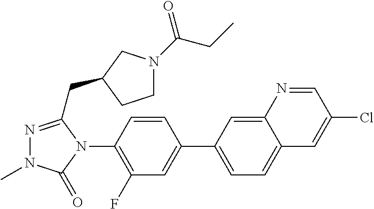 [SMILES: CCC(=O)N1CC[C@@H](Cc2nn(C)c(=O)n2-c2ccc(-c3ccc4cc(Cl)cnc4c3)cc2F)C1] |
| 1021 | 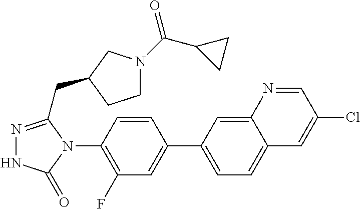 [SMILES: O=C(C1CC1)N1CC[C@@H](Cc2n[nH]c(=O)n2-c2ccc(-c3ccc4cc(Cl)cnc4c3)cc2F)C1] |
| 1022 | 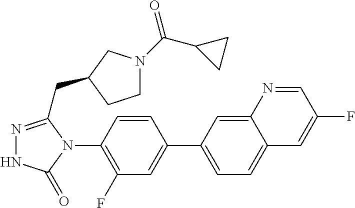 [SMILES: O=C(C1CC1)N1CC[C@@H](Cc2n[nH]c(=O)n2-c2ccc(-c3ccc4cc(F)cnc4c3)cc2F)C1] |
| 1023 | 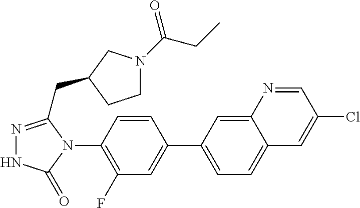 [SMILES: CCC(=O)N1CC[C@@H](Cc2n[nH]c(=O)n2-c2ccc(-c3ccc4cc(Cl)cnc4c3)cc2F)C1] |
| 1024 | 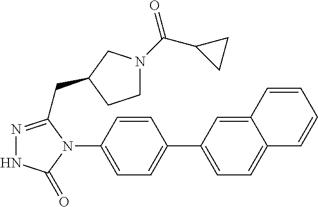 [SMILES: O=C(C1CC1)N1CC[C@@H](Cc2n[nH]c(=O)n2-c2ccc(-c3ccc4ccccc4c3)cc2)C1] |
| 1025 | 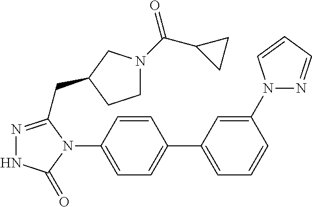 [SMILES: O=C(C1CC1)N1CC[C@@H](Cc2n[nH]c(=O)n2-c2ccc(-c3cccc(-n4cccn4)c3)cc2)C1] |
| 1026 | 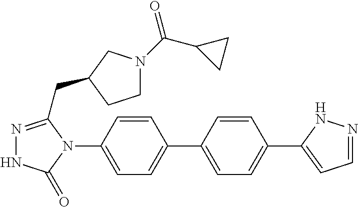 [SMILES: O=C(C1CC1)N1CC[C@@H](Cc2n[nH]c(=O)n2-c2ccc(-c3ccc(-c4ccn[nH]4)cc3)cc2)C1] |
| 1027 | 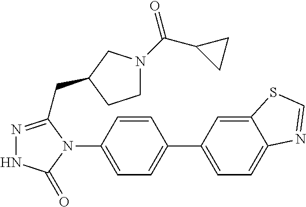 [SMILES: O=C(C1CC1)N1CC[C@@H](Cc2n[nH]c(=O)n2-c2ccc(-c3ccc4ncsc4c3)cc2)C1] |
| 1028 | 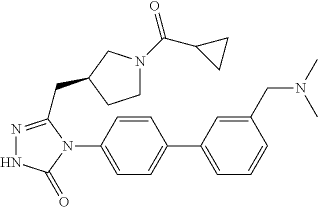 [SMILES: CN(C)Cc1cccc(-c2ccc(-n3c(C[C@@H]4CCN(C(=O)C5CC5)C4)n[nH]c3=O)cc2)c1] |
| 1029 | 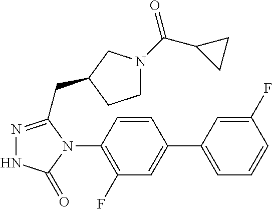 [SMILES: O=C(C1CC1)N1CC[C@@H](Cc2n[nH]c(=O)n2-c2ccc(-c3cccc(F)c3)cc2F)C1] |
| 1030 | 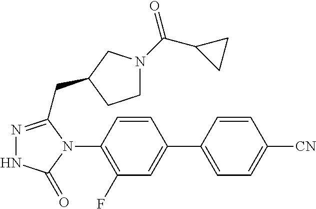 [SMILES: N#Cc1ccc(-c2ccc(-n3c(C[C@@H]4CCN(C(=O)C5CC5)C4)n[nH]c3=O)c(F)c2)cc1] |
| 1031 | 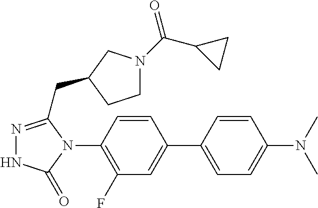 [SMILES: CN(C)c1ccc(-c2ccc(-n3c(C[C@@H]4CCN(C(=O)C5CC5)C4)n[nH]c3=O)c(F)c2)cc1] |
| 1032 | 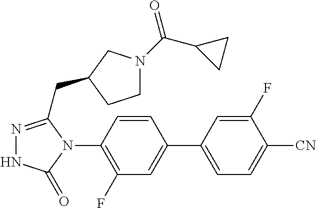 [SMILES: N#Cc1ccc(-c2ccc(-n3c(C[C@@H]4CCN(C(=O)C5CC5)C4)n[nH]c3=O)c(F)c2)cc1F] |
| 1033 | 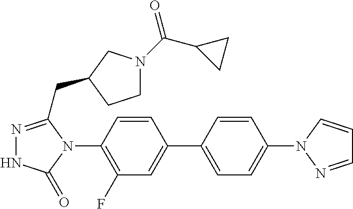 [SMILES: O=C(C1CC1)N1CC[C@@H](Cc2n[nH]c(=O)n2-c2ccc(-c3ccc(-n4cccn4)cc3)cc2F)C1] |
| 1034 | 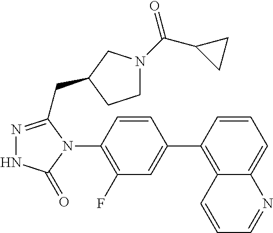 [SMILES: O=C(C1CC1)N1CC[C@@H](Cc2n[nH]c(=O)n2-c2ccc(-c3cccc4ncccc34)cc2F)C1] |
| 1035 | 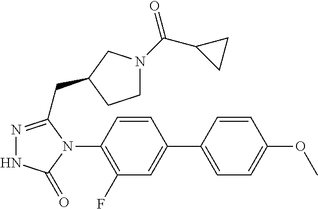 [SMILES: COc1ccc(-c2ccc(-n3c(C[C@@H]4CCN(C(=O)C5CC5)C4)n[nH]c3=O)c(F)c2)cc1] |
| 1036 | 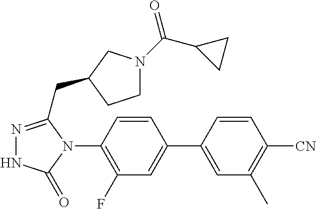 [SMILES: Cc1cc(-c2ccc(-n3c(C[C@@H]4CCN(C(=O)C5CC5)C4)n[nH]c3=O)c(F)c2)ccc1C#N] |
| 1037 | 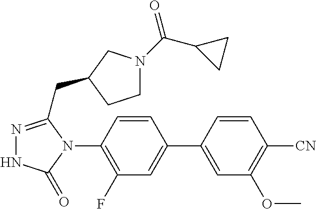 [SMILES: COc1cc(-c2ccc(-n3c(C[C@@H]4CCN(C(=O)C5CC5)C4)n[nH]c3=O)c(F)c2)ccc1C#N] |
| 1038 | 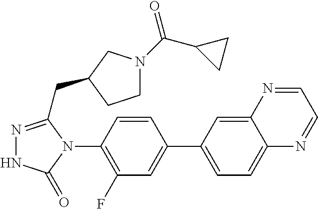 [SMILES: O=C(C1CC1)N1CC[C@@H](Cc2n[nH]c(=O)n2-c2ccc(-c3ccc4nccnc4c3)cc2F)C1] |
| 1039 | 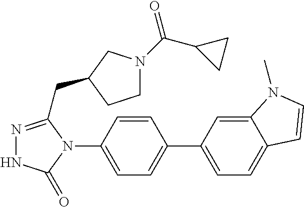 [SMILES: Cn1ccc2ccc(-c3ccc(-n4c(C[C@@H]5CCN(C(=O)C6CC6)C5)n[nH]c4=O)cc3)cc21] |
| 1040 | 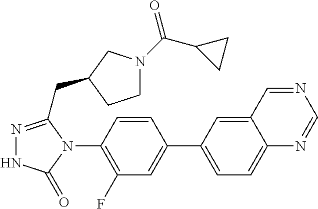 [SMILES: O=C(C1CC1)N1CC[C@@H](Cc2n[nH]c(=O)n2-c2ccc(-c3ccc4ncncc4c3)cc2F)C1] |
| 1041 | 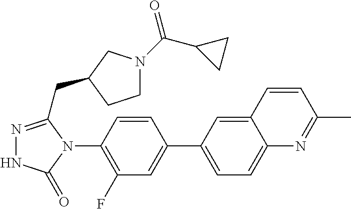 [SMILES: Cc1ccc2cc(-c3ccc(-n4c(C[C@@H]5CCN(C(=O)C6CC6)C5)n[nH]c4=O)c(F)c3)ccc2n1] |
| 1042 | 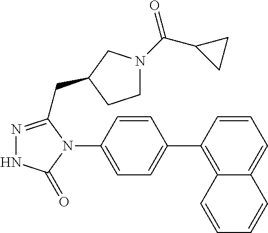 [SMILES: O=C(C1CC1)N1CC[C@@H](Cc2n[nH]c(=O)n2-c2ccc(-c3cccc4ccccc34)cc2)C1] |
| 1043 | 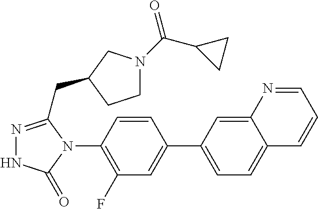 [SMILES: O=C(C1CC1)N1CC[C@@H](Cc2n[nH]c(=O)n2-c2ccc(-c3ccc4cccnc4c3)cc2F)C1] |
| 1044 | 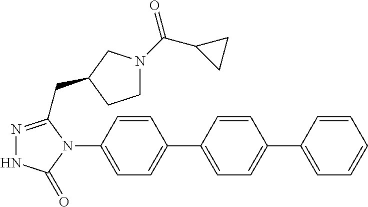 [SMILES: O=C(C1CC1)N1CC[C@@H](Cc2n[nH]c(=O)n2-c2ccc(-c3ccc(-c4ccccc4)cc3)cc2)C1] |
| 1045 | 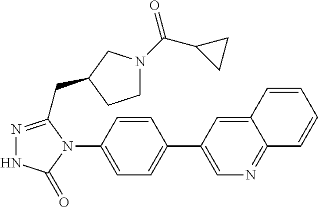 [SMILES: O=C(C1CC1)N1CC[C@@H](Cc2n[nH]c(=O)n2-c2ccc(-c3cnc4ccccc4c3)cc2)C1] |
| 1046 | 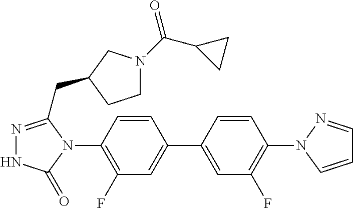 [SMILES: O=C(C1CC1)N1CC[C@@H](Cc2n[nH]c(=O)n2-c2ccc(-c3ccc(-n4cccn4)c(F)c3)cc2F)C1] |
| 1047 | 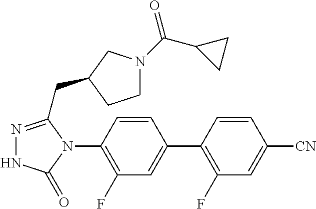 [SMILES: N#Cc1ccc(-c2ccc(-n3c(C[C@@H]4CCN(C(=O)C5CC5)C4)n[nH]c3=O)c(F)c2)c(F)c1] |
| 1048 | 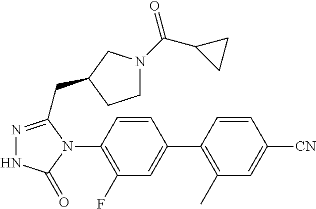 [SMILES: Cc1cc(C#N)ccc1-c1ccc(-n2c(C[C@@H]3CCN(C(=O)C4CC4)C3)n[nH]c2=O)c(F)c1] |
| 1049 | 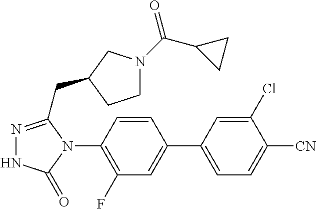 [SMILES: N#Cc1ccc(-c2ccc(-n3c(C[C@@H]4CCN(C(=O)C5CC5)C4)n[nH]c3=O)c(F)c2)cc1Cl] |
| 1050 | 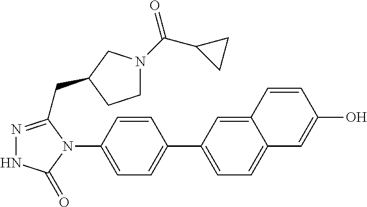 [SMILES: O=C(C1CC1)N1CC[C@@H](Cc2n[nH]c(=O)n2-c2ccc(-c3ccc4cc(O)ccc4c3)cc2)C1] |
| 1051 | 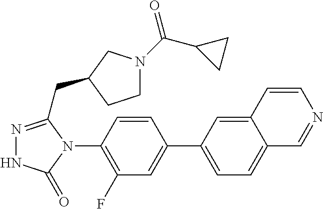 [SMILES: O=C(C1CC1)N1CC[C@@H](Cc2n[nH]c(=O)n2-c2ccc(-c3ccc4cnccc4c3)cc2F)C1] |
| 1052 | 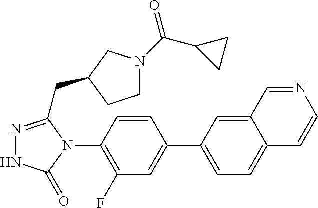 [SMILES: O=C(C1CC1)N1CC[C@@H](Cc2n[nH]c(=O)n2-c2ccc(-c3ccc4ccncc4c3)cc2F)C1] |
| 1053 | 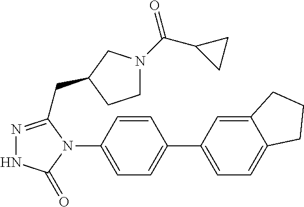 [SMILES: O=C(C1CC1)N1CC[C@@H](Cc2n[nH]c(=O)n2-c2ccc(-c3ccc4c(c3)CCC4)cc2)C1] |
| 1054 | 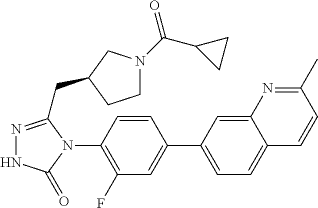 [SMILES: Cc1ccc2ccc(-c3ccc(-n4c(C[C@@H]5CCN(C(=O)C6CC6)C5)n[nH]c4=O)c(F)c3)cc2n1] |
| 1055 | 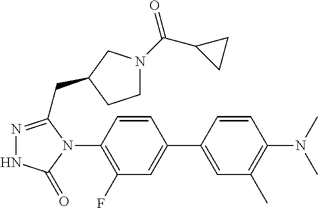 [SMILES: Cc1cc(-c2ccc(-n3c(C[C@@H]4CCN(C(=O)C5CC5)C4)n[nH]c3=O)c(F)c2)ccc1N(C)C] |
| 1056 | 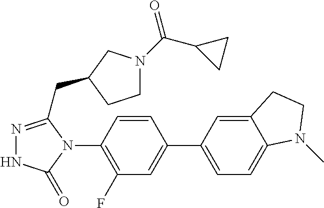 [SMILES: CN1CCc2cc(-c3ccc(-n4c(C[C@@H]5CCN(C(=O)C6CC6)C5)n[nH]c4=O)c(F)c3)ccc21] |
| 1057 | 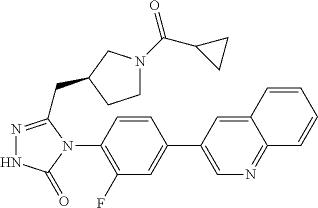 [SMILES: O=C(C1CC1)N1CC[C@@H](Cc2n[nH]c(=O)n2-c2ccc(-c3cnc4ccccc4c3)cc2F)C1] |
| 1058 | 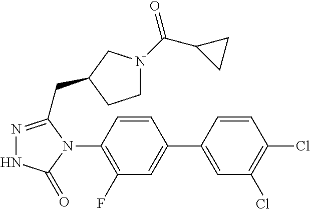 [SMILES: O=C(C1CC1)N1CC[C@@H](Cc2n[nH]c(=O)n2-c2ccc(-c3ccc(Cl)c(Cl)c3)cc2F)C1] |
| 1059 | 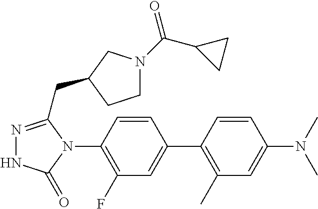 [SMILES: Cc1cc(N(C)C)ccc1-c1ccc(-n2c(C[C@@H]3CCN(C(=O)C4CC4)C3)n[nH]c2=O)c(F)c1] |
| 1060 | 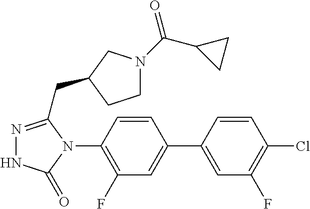 [SMILES: O=C(C1CC1)N1CC[C@@H](Cc2n[nH]c(=O)n2-c2ccc(-c3ccc(Cl)c(F)c3)cc2F)C1] |
| 1061 | 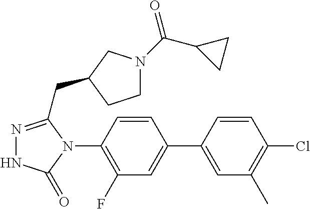 [SMILES: Cc1cc(-c2ccc(-n3c(C[C@@H]4CCN(C(=O)C5CC5)C4)n[nH]c3=O)c(F)c2)ccc1Cl] |
| 1062 | 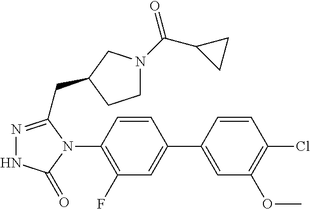 [SMILES: COc1cc(-c2ccc(-n3c(C[C@@H]4CCN(C(=O)C5CC5)C4)n[nH]c3=O)c(F)c2)ccc1Cl] |
| 1063 | 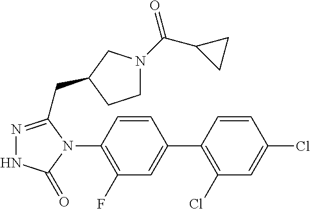 [SMILES: O=C(C1CC1)N1CC[C@@H](Cc2n[nH]c(=O)n2-c2ccc(-c3ccc(Cl)cc3Cl)cc2F)C1] |
| 1064 | 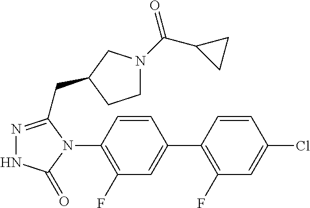 [SMILES: O=C(C1CC1)N1CC[C@@H](Cc2n[nH]c(=O)n2-c2ccc(-c3ccc(Cl)cc3F)cc2F)C1] |
| 1065 | 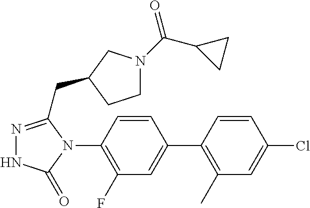 [SMILES: Cc1cc(Cl)ccc1-c1ccc(-n2c(C[C@@H]3CCN(C(=O)C4CC4)C3)n[nH]c2=O)c(F)c1] |
| 1066 | 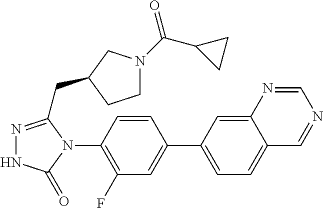 [SMILES: O=C(C1CC1)N1CC[C@@H](Cc2n[nH]c(=O)n2-c2ccc(-c3ccc4cncnc4c3)cc2F)C1] |
| 1067 | 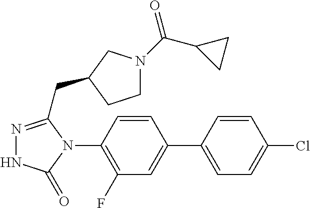 [SMILES: O=C(C1CC1)N1CC[C@@H](Cc2n[nH]c(=O)n2-c2ccc(-c3ccc(Cl)cc3)cc2F)C1] |
| 1068 | 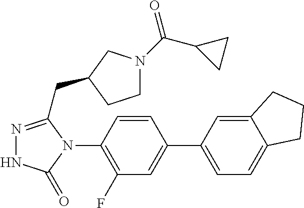 [SMILES: O=C(C1CC1)N1CC[C@@H](Cc2n[nH]c(=O)n2-c2ccc(-c3ccc4c(c3)CCC4)cc2F)C1] |
| 1069 | 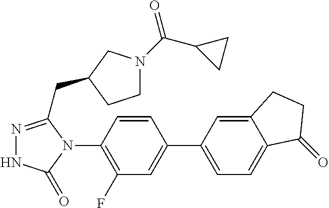 [SMILES: O=C1CCc2cc(-c3ccc(-n4c(C[C@@H]5CCN(C(=O)C6CC6)C5)n[nH]c4=O)c(F)c3)ccc21] |
| 1070 | 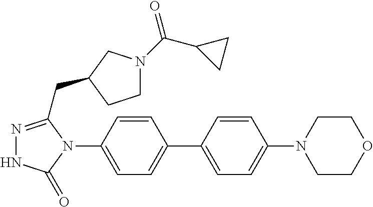 [SMILES: O=C(C1CC1)N1CC[C@@H](Cc2n[nH]c(=O)n2-c2ccc(-c3ccc(N4CCOCC4)cc3)cc2)C1] |
| 1071 | 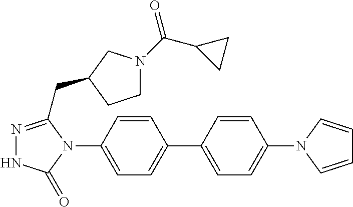 [SMILES: O=C(C1CC1)N1CC[C@@H](Cc2n[nH]c(=O)n2-c2ccc(-c3ccc(-n4cccc4)cc3)cc2)C1] |
| 1072 | 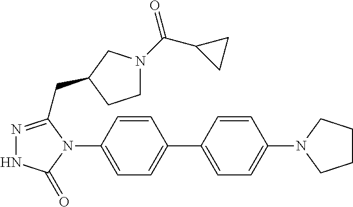 [SMILES: O=C(C1CC1)N1CC[C@@H](Cc2n[nH]c(=O)n2-c2ccc(-c3ccc(N4CCCC4)cc3)cc2)C1] |
| 1073 | 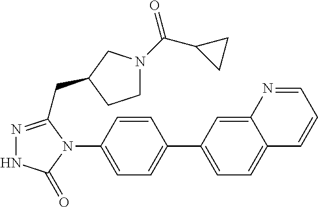 [SMILES: O=C(C1CC1)N1CC[C@@H](Cc2n[nH]c(=O)n2-c2ccc(-c3ccc4cccnc4c3)cc2)C1] |
| 1074 | 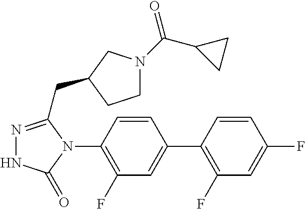 [SMILES: O=C(C1CC1)N1CC[C@@H](Cc2n[nH]c(=O)n2-c2ccc(-c3ccc(F)cc3F)cc2F)C1] |
| 1075 | 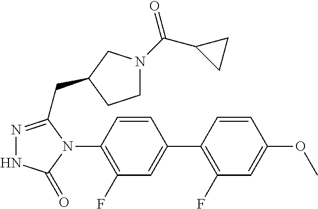 [SMILES: COc1ccc(-c2ccc(-n3c(C[C@@H]4CCN(C(=O)C5CC5)C4)n[nH]c3=O)c(F)c2)c(F)c1] |
| 1076 | 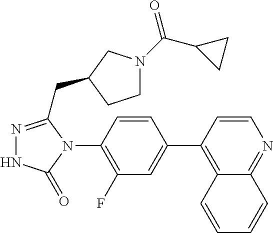 [SMILES: O=C(C1CC1)N1CC[C@@H](Cc2n[nH]c(=O)n2-c2ccc(-c3ccnc4ccccc34)cc2F)C1] |
| 1077 | 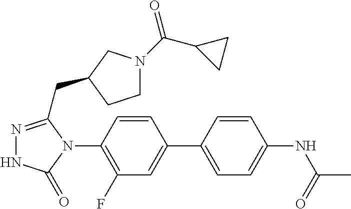 [SMILES: CC(=O)Nc1ccc(-c2ccc(-n3c(C[C@@H]4CCN(C(=O)C5CC5)C4)n[nH]c3=O)c(F)c2)cc1] |
| 1078 | 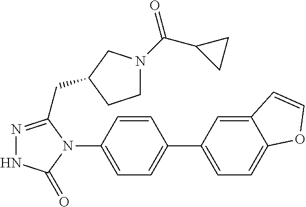 [SMILES: O=C(C1CC1)N1CC[C@H](Cc2n[nH]c(=O)n2-c2ccc(-c3ccc4occc4c3)cc2)C1] |
| 1079 | 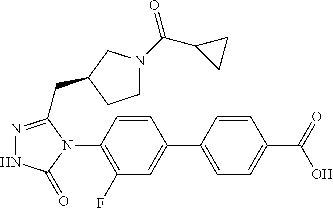 [SMILES: O=C(O)c1ccc(-c2ccc(-n3c(C[C@@H]4CCN(C(=O)C5CC5)C4)n[nH]c3=O)c(F)c2)cc1] |
| 1080 | 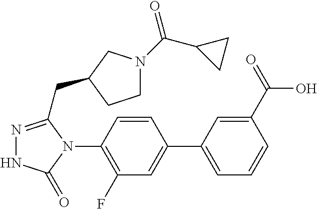 [SMILES: O=C(O)c1cccc(-c2ccc(-n3c(C[C@@H]4CCN(C(=O)C5CC5)C4)n[nH]c3=O)c(F)c2)c1] |
| 1081 | 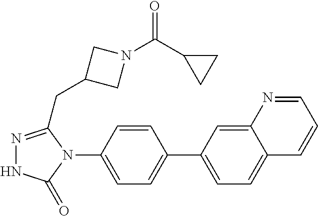 [SMILES: O=C(C1CC1)N1CC(Cc2n[nH]c(=O)n2-c2ccc(-c3ccc4cccnc4c3)cc2)C1] |
| 1082 | 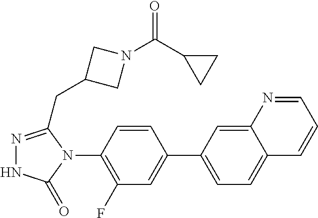 [SMILES: O=C(C1CC1)N1CC(Cc2n[nH]c(=O)n2-c2ccc(-c3ccc4cccnc4c3)cc2F)C1] |
| 1083 | 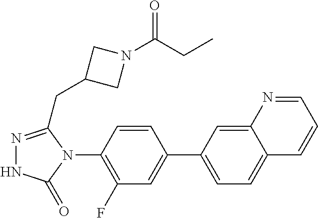 [SMILES: CCC(=O)N1CC(Cc2n[nH]c(=O)n2-c2ccc(-c3ccc4cccnc4c3)cc2F)C1] |
| 1084 | 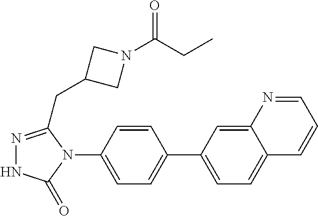 [SMILES: CCC(=O)N1CC(Cc2n[nH]c(=O)n2-c2ccc(-c3ccc4cccnc4c3)cc2)C1] |
| 1085 | 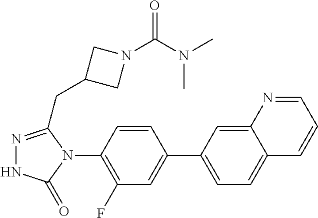 [SMILES: CN(C)C(=O)N1CC(Cc2n[nH]c(=O)n2-c2ccc(-c3ccc4cccnc4c3)cc2F)C1] |
| 1086 | 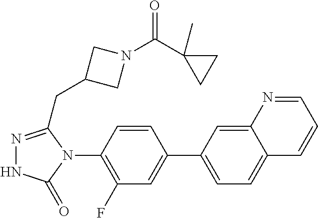 [SMILES: CC1(C(=O)N2CC(Cc3n[nH]c(=O)n3-c3ccc(-c4ccc5cccnc5c4)cc3F)C2)CC1] |
| 1087 | 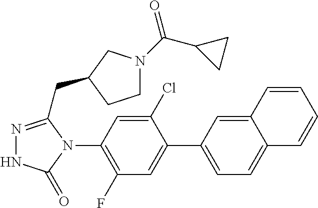 [SMILES: O=C(C1CC1)N1CC[C@@H](Cc2n[nH]c(=O)n2-c2cc(Cl)c(-c3ccc4ccccc4c3)cc2F)C1] |
| 1088 | 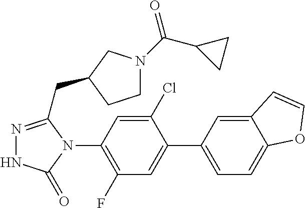 [SMILES: O=C(C1CC1)N1CC[C@@H](Cc2n[nH]c(=O)n2-c2cc(Cl)c(-c3ccc4occc4c3)cc2F)C1] |
| 1089 | 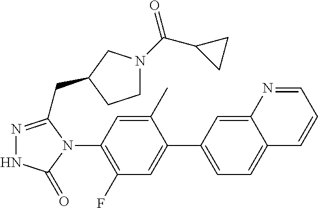 [SMILES: Cc1cc(-n2c(C[C@@H]3CCN(C(=O)C4CC4)C3)n[nH]c2=O)c(F)cc1-c1ccc2cccnc2c1] |
| 1090 | 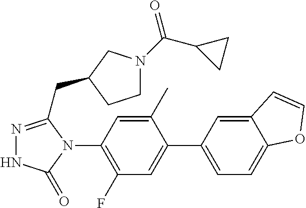 [SMILES: Cc1cc(-n2c(C[C@@H]3CCN(C(=O)C4CC4)C3)n[nH]c2=O)c(F)cc1-c1ccc2occc2c1] |
| 1091 | 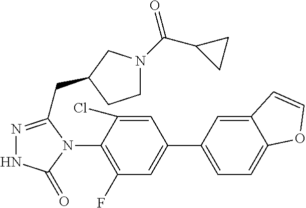 [SMILES: O=C(C1CC1)N1CC[C@@H](Cc2n[nH]c(=O)n2-c2c(F)cc(-c3ccc4occc4c3)cc2Cl)C1] |
| 1092 | 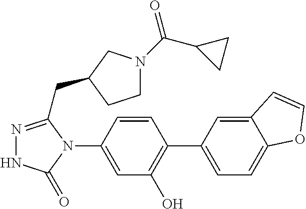 [SMILES: O=C(C1CC1)N1CC[C@@H](Cc2n[nH]c(=O)n2-c2ccc(-c3ccc4occc4c3)c(O)c2)C1] |
| 1093 | 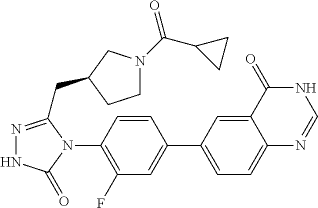 [SMILES: O=C(C1CC1)N1CC[C@@H](Cc2n[nH]c(=O)n2-c2ccc(-c3ccc4nc[nH]c(=O)c4c3)cc2F)C1] |
| 1094 | 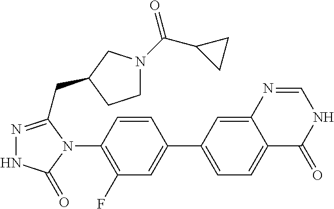 [SMILES: O=C(C1CC1)N1CC[C@@H](Cc2n[nH]c(=O)n2-c2ccc(-c3ccc4c(=O)[nH]cnc4c3)cc2F)C1] |
| 1095 | 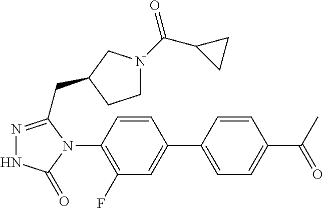 [SMILES: CC(=O)c1ccc(-c2ccc(-n3c(C[C@@H]4CCN(C(=O)C5CC5)C4)n[nH]c3=O)c(F)c2)cc1] |
| 1096 | 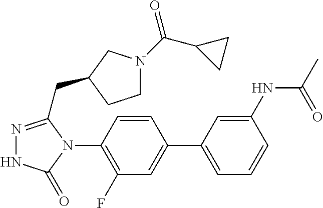 [SMILES: CC(=O)Nc1cccc(-c2ccc(-n3c(C[C@@H]4CCN(C(=O)C5CC5)C4)n[nH]c3=O)c(F)c2)c1] |
| 1097 | 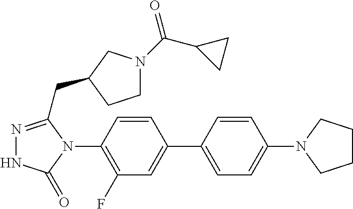 [SMILES: O=C(C1CC1)N1CC[C@@H](Cc2n[nH]c(=O)n2-c2ccc(-c3ccc(N4CCCC4)cc3)cc2F)C1] |
| 1098 | 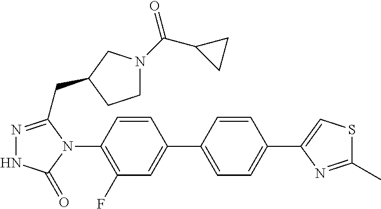 [SMILES: Cc1nc(-c2ccc(-c3ccc(-n4c(C[C@@H]5CCN(C(=O)C6CC6)C5)n[nH]c4=O)c(F)c3)cc2)cs1] |
| 1099 | 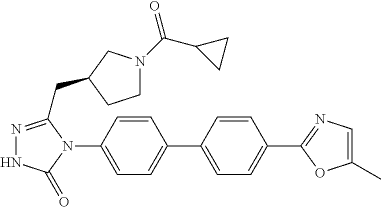 [SMILES: Cc1cnc(-c2ccc(-c3ccc(-n4c(C[C@@H]5CCN(C(=O)C6CC6)C5)n[nH]c4=O)cc3)cc2)o1] |
| 1100 | 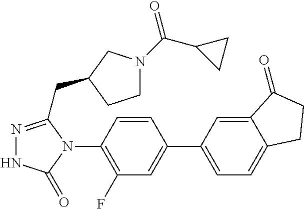 [SMILES: O=C1CCc2ccc(-c3ccc(-n4c(C[C@@H]5CCN(C(=O)C6CC6)C5)n[nH]c4=O)c(F)c3)cc21] |
| 1101 | 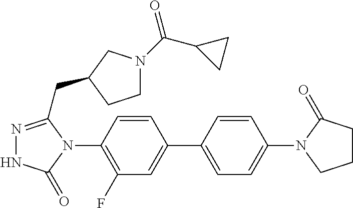 [SMILES: O=C(C1CC1)N1CC[C@@H](Cc2n[nH]c(=O)n2-c2ccc(-c3ccc(N4CCCC4=O)cc3)cc2F)C1] |
| 1102 | 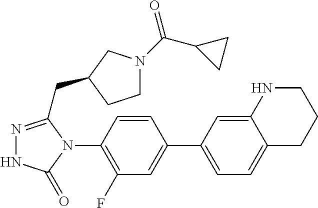 [SMILES: O=C(C1CC1)N1CC[C@@H](Cc2n[nH]c(=O)n2-c2ccc(-c3ccc4c(c3)NCCC4)cc2F)C1] |
| 1103 | 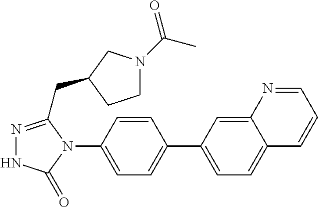 [SMILES: CC(=O)N1CC[C@@H](Cc2n[nH]c(=O)n2-c2ccc(-c3ccc4cccnc4c3)cc2)C1] |
| 1104 | 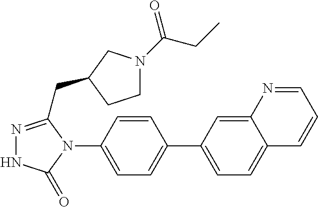 [SMILES: CCC(=O)N1CC[C@@H](Cc2n[nH]c(=O)n2-c2ccc(-c3ccc4cccnc4c3)cc2)C1] |
| 1105 | 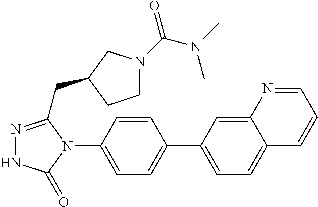 [SMILES: CN(C)C(=O)N1CC[C@@H](Cc2n[nH]c(=O)n2-c2ccc(-c3ccc4cccnc4c3)cc2)C1] |
| 1106 | 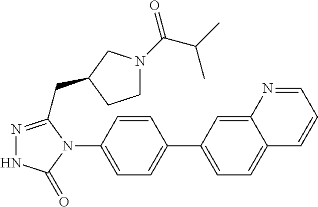 [SMILES: CC(C)C(=O)N1CC[C@@H](Cc2n[nH]c(=O)n2-c2ccc(-c3ccc4cccnc4c3)cc2)C1] |
| 1107 | 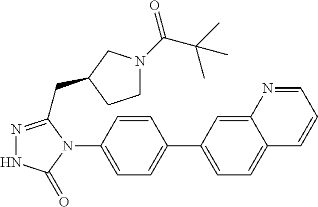 [SMILES: CC(C)(C)C(=O)N1CC[C@@H](Cc2n[nH]c(=O)n2-c2ccc(-c3ccc4cccnc4c3)cc2)C1] |
| 1108 | 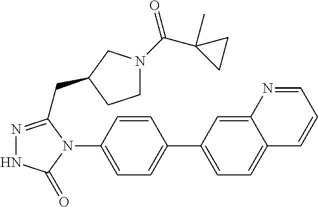 [SMILES: CC1(C(=O)N2CC[C@@H](Cc3n[nH]c(=O)n3-c3ccc(-c4ccc5cccnc5c4)cc3)C2)CC1] |
| 1109 | 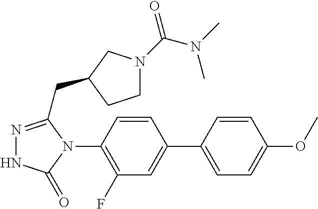 [SMILES: COc1ccc(-c2ccc(-n3c(C[C@@H]4CCN(C(=O)N(C)C)C4)n[nH]c3=O)c(F)c2)cc1] |
| 1110 | 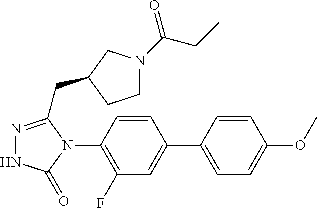 [SMILES: CCC(=O)N1CC[C@@H](Cc2n[nH]c(=O)n2-c2ccc(-c3ccc(OC)cc3)cc2F)C1] |
| 1111 | 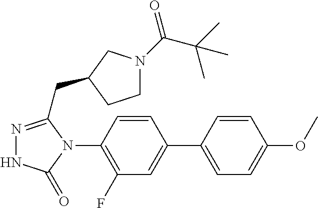 [SMILES: COc1ccc(-c2ccc(-n3c(C[C@@H]4CCN(C(=O)C(C)(C)C)C4)n[nH]c3=O)c(F)c2)cc1] |
| 1112 | 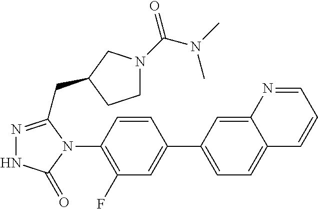 [SMILES: CN(C)C(=O)N1CC[C@@H](Cc2n[nH]c(=O)n2-c2ccc(-c3ccc4cccnc4c3)cc2F)C1] |
| 1113 | 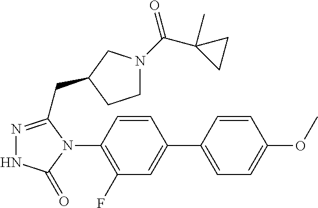 [SMILES: COc1ccc(-c2ccc(-n3c(C[C@@H]4CCN(C(=O)C5(C)CC5)C4)n[nH]c3=O)c(F)c2)cc1] |
| 1114 | 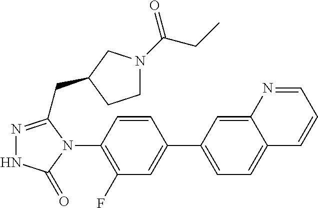 [SMILES: CCC(=O)N1CC[C@@H](Cc2n[nH]c(=O)n2-c2ccc(-c3ccc4cccnc4c3)cc2F)C1] |
| 1115 | 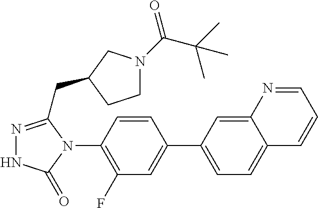 [SMILES: CC(C)(C)C(=O)N1CC[C@@H](Cc2n[nH]c(=O)n2-c2ccc(-c3ccc4cccnc4c3)cc2F)C1] |
| 1116 | 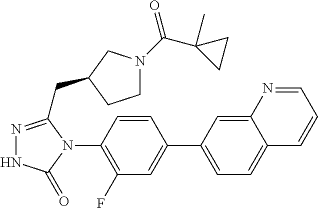 [SMILES: CC1(C(=O)N2CC[C@@H](Cc3n[nH]c(=O)n3-c3ccc(-c4ccc5cccnc5c4)cc3F)C2)CC1] |
| 1117 | 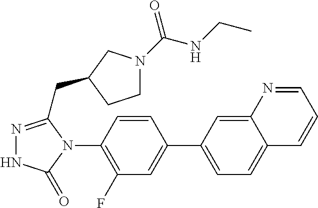 [SMILES: CCNC(=O)N1CC[C@@H](Cc2n[nH]c(=O)n2-c2ccc(-c3ccc4cccnc4c3)cc2F)C1] |
| 1118 | 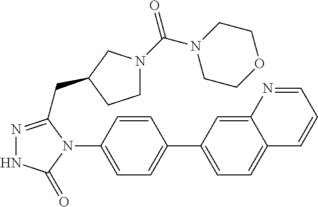 [SMILES: O=C(N1CCOCC1)N1CC[C@@H](Cc2n[nH]c(=O)n2-c2ccc(-c3ccc4cccnc4c3)cc2)C1] |
| 1119 | 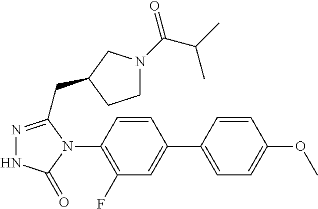 [SMILES: COc1ccc(-c2ccc(-n3c(C[C@@H]4CCN(C(=O)C(C)C)C4)n[nH]c3=O)c(F)c2)cc1] |
| 1120 | 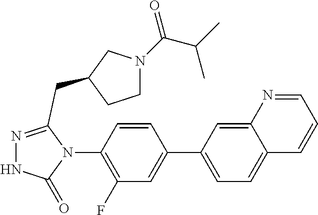 [SMILES: CC(C)C(=O)N1CC[C@@H](Cc2n[nH]c(=O)n2-c2ccc(-c3ccc4cccnc4c3)cc2F)C1] |
| 1121 | 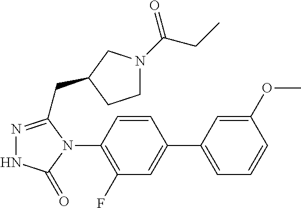 [SMILES: CCC(=O)N1CC[C@@H](Cc2n[nH]c(=O)n2-c2ccc(-c3cccc(OC)c3)cc2F)C1] |
| 1122 | 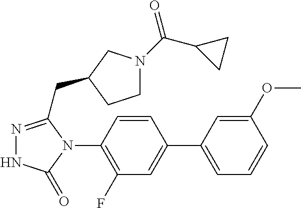 [SMILES: COc1cccc(-c2ccc(-n3c(C[C@@H]4CCN(C(=O)C5CC5)C4)n[nH]c3=O)c(F)c2)c1] |
| 1123 | 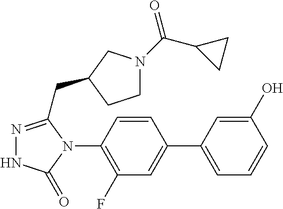 [SMILES: O=C(C1CC1)N1CC[C@@H](Cc2n[nH]c(=O)n2-c2ccc(-c3cccc(O)c3)cc2F)C1] |
| 1124 | 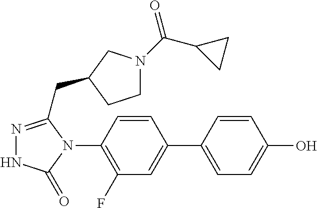 [SMILES: O=C(C1CC1)N1CC[C@@H](Cc2n[nH]c(=O)n2-c2ccc(-c3ccc(O)cc3)cc2F)C1] |
| 1125 | 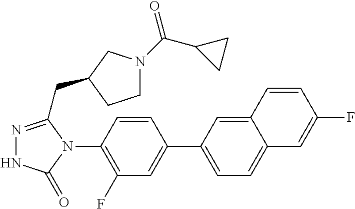 [SMILES: O=C(C1CC1)N1CC[C@@H](Cc2n[nH]c(=O)n2-c2ccc(-c3ccc4cc(F)ccc4c3)cc2F)C1] |
| 1126 | 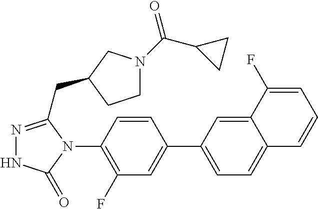 [SMILES: O=C(C1CC1)N1CC[C@@H](Cc2n[nH]c(=O)n2-c2ccc(-c3ccc4cccc(F)c4c3)cc2F)C1] |
| 1127 | 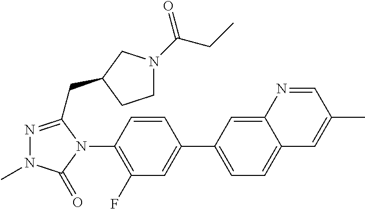 |
| 1128 | 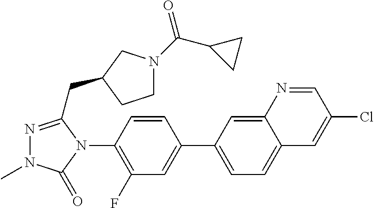 [SMILES: Cn1nc(C[C@@H]2CCN(C(=O)C3CC3)C2)n(-c2ccc(-c3ccc4cc(Cl)cnc4c3)cc2F)c1=O] |
| 1129 | 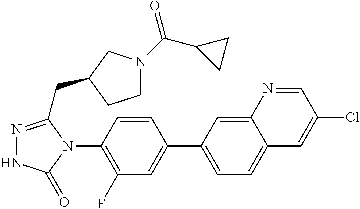 [SMILES: O=C(C1CC1)N1CC[C@@H](Cc2n[nH]c(=O)n2-c2ccc(-c3ccc4cc(Cl)cnc4c3)cc2F)C1] |
| 1130 | 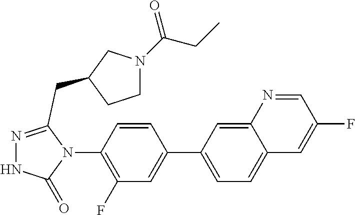 [SMILES: CCC(=O)N1CC[C@@H](Cc2n[nH]c(=O)n2-c2ccc(-c3ccc4cc(F)cnc4c3)cc2F)C1] |
| 1131 | 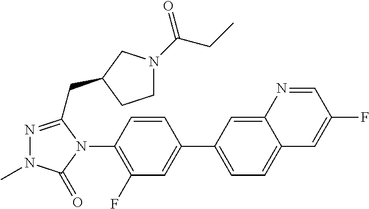 [SMILES: CCC(=O)N1CC[C@@H](Cc2nn(C)c(=O)n2-c2ccc(-c3ccc4cc(F)cnc4c3)cc2F)C1] |

In some embodiments, the compound has the structure of formula (XXIII):

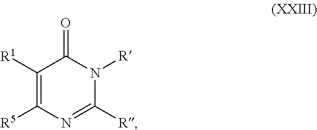

wherein one of R′ and R″ is

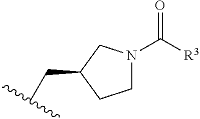

and the other of R′ and R″ is

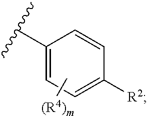

wherein R¹ and R⁵ are each independently selected from the group consisting of: hydrogen, C₁-C₆alkyl, —C₁-C₆ alkoxy, hydroxyl, halogen, —NR⁷R⁸, —C₁-C₆alkylNR⁷R⁸, cyano, C₄-C₆ heterocycloalkyl, —OC₁-C₄alkyl, and —C(O)NRₐR_{b}, in which Rₐ and R_{b} are independently hydrogen, C₁-C₆alkyl, or C₃-C₇ cycloalkyl, or Rₐ and R_{b} taken together with the atoms to which they are connected form a C₄-C₆ heterocycloalkyl; R⁷ is selected from the group consisting of hydrogen, C₁-C₄alkyl, C₃-C₇ cycloalkyl, —C₁-C₃alkyl C₃-C₇cycloalkyl, phenyl, and —C₁-C₃ alkylphenyl; R⁸ is hydrogen, C₁-C₄ alkyl, C₃-C₇ cycloalkyl, or —C₁-C₃ alkyl C₃-C₇ cycloalkyl; or R¹ and R⁵ taken together with the atoms to which they are connected form a 5- or 6-membered ring, which ring optionally contains one or two heteroatoms and is optionally substituted by 1 to 2 groups selected from: halogen, C₁-C₄ alkoxy, and C₁-C₄ alkyl; R₂ is phenyl, 5- or 6-membered heteroaryl, naphthyl, or 9- or 10-membered heterocyclyl; wherein said phenyl, 5- or 6-membered heteroaryl, naphthyl, 9- or 10-membered heterocyclyl, is optionally substituted with 1 to 3 substituents independently selected from halogen, C₁-C₄ alkyl, —CF₃, C₃-C₇ cycloalkyl, —C(O)C₁-C₄ alkyl, —C(O)C₃-C₇ cycloalkyl, —CO(phenyl), —C₁-C₄(═O)OH, —C(═O)OC₁-C₄ alkyl, —CONR⁷R⁸, phenyl, —SO₂C₁-C₄ alkyl, —SO₂NR⁷R⁸, cyano, oxo, hydroxyl, C₁-C₄ alkoxy, C₃-C₇ cycloalkoxy, hydroxyC₁-C₄ alkyl-, C₁-C₄ alkoxy C₁-C₄ alkyl-, —OCF₃, —NR⁷R⁸, R⁷R⁸NC₁-C₄ alkyl-, —NR⁷C(O)C₁-C₄ alkyl, —NR⁷CONR⁷R⁸, —NR⁷SO₂C₁-C₄ alkyl, —NR⁷SO₂NR⁷R⁸, and R⁹; R⁹ is a 5- or 6-membered heteroaryl ring containing 1 to 4 heteroatoms selected from oxygen, nitrogen, and sulfur, which is optionally substituted with 1 or 2 substituents selected from halogen, C₁-C₄ alkyl, CF₃, C₁-C₄ alkoxy, and —NR⁷R⁸; R³ is selected from the group consisting of C₁-C₆ alkyl, —CF₃, C₃-C₇ cycloalkyl, C₁-C₄ alkoxy, OC₁₋₆ alkyl, R⁷R⁸NC₁-C₄ alkyl-, and —NR⁷R⁸; wherein said C₃-C₇ cycloalkyl is optionally substituted 1 or 2 times independently by halogen or C₁-C₄alkyl; each R⁴ is selected from the group consisting of: hydroxyl, C₁-C₆ alkyl, C₁-C₆ alkoxy and halogen; m is 0 to 3; or a pharmaceutically acceptable salt thereof.

In some embodiments, the compound has the structure of formula (XXIII-A):

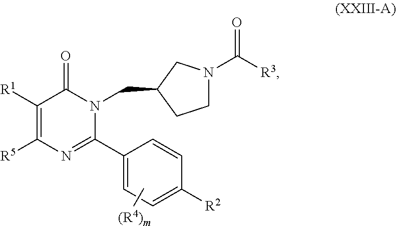

wherein R¹ and R⁵ are each independently selected from the group consisting of: hydrogen, C₁-C₆ alkyl, —C₁-C₆ alkoxy, hydroxyl, halogen, —NR⁷R⁸, —C₁-C₆ alkylNR⁷R⁸, cyano, C₄-C₆ heterocycloalkyl, —OC₁-C₄ alkyl, and —C(O)NRₐR_{b}, in which Rₐ and R_{b} are independently hydrogen, C₁-C₆ alkyl, or C₃-C₇ cycloalkyl, or Rₐ and R_{b} taken together with the atoms to which they are connected form a C₄-C₆ heterocycloalkyl; R⁷ is selected from the group consisting of hydrogen, C₁-C₄alkyl, C₃-C₇ cycloalkyl, —C₁-C₃alkyl C₃-C₇cycloalkyl, phenyl, and —C₁-C₃ alkylphenyl; R⁸ is hydrogen, C₁-C₄ alkyl, C₃-C₇ cycloalkyl, or —C₁-C₃ alkyl C₃-C₇ cycloalkyl; or R¹ and R⁵ taken together with the atoms to which they are connected form a 5- or 6-membered ring, in which the ring optionally contains one or two heteroatoms and is optionally substituted by 1 to 2 groups selected from: halogen, C₁-C₄ alkoxy, and C₁-C₄ alkyl; R² is selected from the group consisting of: phenyl, 5- or 6-membered heteroaryl, naphthyl, or 9- or 10-membered heterocyclyl; wherein said phenyl, 5- or 6-membered heteroaryl, naphthyl, 9- or 10-membered heterocyclyl, is optionally substituted with 1 to 3 substituents independently selected from halogen, C₁-C₄alkyl, —CF₃, C₃-C₇ cycloalkyl, —C(O)C₁-C₄ alkyl, —C(O)C₃-C₇ cycloalkyl, —CO(phenyl), —C₁-C₄(═O)OH, —C(═O)OC₁-C₄ alkyl, —CONR⁷R⁸, phenyl, —SO₂C₁-C₄ alkyl, —SO₂NR⁷R⁸, cyano, oxo, hydroxyl, C₁-C₄ alkoxy, C₃-C₇ cycloalkoxy, hydroxyC₁-C₄ alkyl-, C₁-C₄ alkoxy C₁-C₄ alkyl-, —OCF₃, —NR⁷R⁸, R⁷R⁸NC₁-C₄ alkyl-, —NR⁷C(O)C₁-C₄ alkyl, —NR⁷CONR⁷R⁸, —NR⁷SO₂C₁-C₄ alkyl, —NR⁷SO₂NR⁷R⁸, and R⁹; R⁹ is a 5- or 6-membered heteroaryl ring containing 1 to 4 heteroatoms selected from oxygen, nitrogen, and sulfur, which is optionally substituted with 1 or 2 substituents selected from halogen, C₁-C₄alkyl, CF₃, C₁-C₄alkoxy, and —NR⁷R⁸; R³ is selected from the group consisting of C₁-C₆alkyl, —CF₃, C₃-C₇cycloalkyl, C₁-C₄ alkoxy, OC₁-C₆alkyl, R⁷R⁸NC₁-C₄ alkyl-, and —NR⁷R⁸; wherein said C₃-C₇cycloalkyl is optionally substituted 1 or 2 times independently by halogen or C₁-C₄ alkyl; each R₄ is selected from the group consisting of: hydroxyl, C₁-C₆alkyl, alkoxy and halogen; m is 0 to 3; or a pharmaceutically acceptable salt thereof.

In some embodiments, the compound has the structure of formula (XXIII-B):

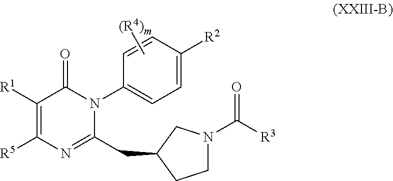

wherein R¹ and R₅ are each independently selected from the group consisting of: hydrogen, C₁-C₆ alkyl, —C₁-C₆ alkoxy, hydroxyl, halogen, —NR⁷R⁸, —C₁₋₆ alkylNR⁷R⁸, cyano, C₄-C₆ heterocycloalkyl, —OC₁-C₄ alkyl, and —C(O)NRₐR_{b}, in which Rₐ and R_{b} are independently hydrogen, C₁-C₆alkyl, or C₃-C₇cycloalkyl, or Rₐ and R_{b} taken together with the atoms to which they are connected form a C₄-C₆ heterocycloalkyl; R⁷ is selected from the group consisting of hydrogen, C₁-C₄alkyl, C₃-C₇ cycloalkyl, —C₁-C₃alkylC₃-C₇cycloalkyl, phenyl, and —C₁-C₃ alkylphenyl; R⁸ is hydrogen, C₁-C₄ alkyl, C₃-C₇ cycloalkyl, or —C₁-C₃ alkyl C₃-C₇ cycloalkyl; or R₁ and R₅ taken together with the atoms to which they are connected form a 5- or 6-membered ring, which ring optionally contains one or two heteroatoms and is optionally substituted by 1 to 2 groups selected from: halogen, C₁-C₄ alkoxy, and C₁-C₄ alkyl; R² is phenyl, 5- or 6-membered heteroaryl, naphthyl, or 9- or 10-membered heterocyclyl; wherein said phenyl, 5- or 6-membered heteroaryl, naphthyl, 9- or 10-membered heterocyclyl, is optionally substituted with 1 to 3 substituents independently selected from halogen, C₁-C₄ alkyl, —CF₃, C₃-C₇ cycloalkyl, —C(O)C₁-C₄ alkyl, —C(O)C₃-C₇ cycloalkyl, —CO(phenyl), —C₁-C₄(═O)OH, —C(═O)OC₁-C₄ alkyl, —CONR⁷R⁸, phenyl, —SO₂C₁-C₄alkyl, —SO₂NR⁷R⁸, cyano, oxo, hydroxyl, C₁-C₄ alkoxy, C₃-C₇ cycloalkoxy, hydroxy C₁-C₄ alkyl-, C₁-C₄ alkoxy C₁-C₄ alkyl-, —OCF₃, —NR⁷R⁸, R⁷R⁸NC₁-C₄ alkyl-, —NR⁷C(O)C₁-C₄ alkyl, —NR⁷CONR⁷R⁸, —NR⁷SO₂C₁-C₄ alkyl, —NR⁷SO₂NR⁷R⁸, and R⁹; R⁹ is a 5- or 6-membered heteroaryl ring containing 1 to 4 heteroatoms selected from oxygen, nitrogen, and sulfur, which is optionally substituted with 1 or 2 substituents selected from halogen, C₁-C₄ alkyl, CF₃, C₁-C₄ alkoxy, and —NR⁷R⁸; R³ is selected from the group consisting of C₁-C₆alkyl, —CF₃, C₃-C₇ cycloalkyl, C₁-C₄ alkoxy, OC₁₋₆ alkyl, R⁷R⁸NC₁-C₄ alkyl-, and —NR⁷R⁸; wherein said C₃-C₇ cycloalkyl is optionally substituted 1 or 2 times independently by halogen or C₁-C₄ alkyl; each R₄ is selected from the group consisting of: hydroxyl, C₁-C₆ alkyl, C₁-C₆alkoxy and halogen; m is 0 to 3; or a pharmaceutically acceptable salt thereof.

In some embodiments, R³ is cyclopropyl. In some embodiments, R¹ and R⁵ are each independently selected from the group consisting of: hydrogen, C₁-C₆ alkyl, C₁-C₆ alkoxy, hydroxyl, halogen, —NR⁷R⁸, cyano, heterocycloalkyl and —C(O)NRₐR_{b}, in which Rₐ and R_{b} are hydrogen, C₁-C₆ alkyl, C₃-C₇cycloalkyl. In some embodiments, R¹ and R⁵ taken together with the atoms to which they are connected form a 5- or 6-membered ring, which ring optionally contains one or two heteroatoms atoms and is optionally substituted by 1 to 2 groups selected from: halogen, C₁-C₆ alkoxy, and C₁-C₆ alkyl. In some embodiments, m is 0. In some embodiments m is 1. In some embodiments, R² is phenyl optionally substituted with 1 to 3 substituents independently selected from halogen, C₁-C₄ alkyl, —CF₃, C₃-C₇ cycloalkyl, —C(O)C₁-C₄ alkyl, —C(O)C₃-C₇ cycloalkyl, —CO(phenyl), —C₁-C₄(═O)OH, —C(═O)OC₁-C₄ alkyl, —CONR⁵R⁶, phenyl, —SO₂C₁-C₄ alkyl, —SO₂NR⁵R⁶, cyano, oxo, hydroxyl, C₁-C₄ alkoxy, C₃-C₇ cycloalkoxy, hydroxyC₁-C₄ alkyl-, C₁-C₄ alkoxy C₁-C₄ alkyl-, —OCF₃, —NR⁵R⁶, R⁵R⁶NC₁-C₄ alkyl-, —NHC(O)C₁-C₄ alkyl, —NHCONR⁵R⁶, —NHSO₂C₁-C₄ alkyl, —NHSO₂NR⁵R⁶, and R⁹. In some embodiments, R² is selected from furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, wherein said furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, and triazinyl, all of which are optionally substituted with 1 to 3 substituents independently selected from halogen, C₁-C₄ alkyl, —CF₃, C₃-C₇ cycloalkyl, —C(O)C₁-C₄ alkyl, —C(O)C₃-C₇ cycloalkyl, —C(O)phenyl, —C₁-C₄(═O)OH, —C(═O)OC₁-C₄ alkyl, —CO₂C₁-C₄ alkyl, —C(O)NR⁵R⁶, phenyl, —SO₂C₁-C₄ alkyl, —SO₂NR⁵R⁶, cyano, oxo, hydroxyl, C₁-C₄ alkoxy, C₃-C₇ cycloalkoxy, hydroxyC₁-C₄ alkyl-, C₁-C₄ alkoxy C₁-C₄ alkyl-, —OCF₃, —NR⁵R⁶, R⁵R⁶NC₁-C₄ alkyl-, —NHC(O)C₁-C₄ alkyl, —NHCONR⁵R⁶, —NHSO₂C₁-C₄ alkyl, and —NHSO₂NR⁵R⁶. In some embodiments, R² is naphthyl optionally substituted with 1 to 3 substituents independently selected from halogen, C₁-C₄alkyl, —CF₃, C₃-C₇ cycloalkyl, —C(O)C₁-C₄alkyl, —C(O)C₃-C₇cycloalkyl, —CO(phenyl), —C₁-C₄(═O)OH, —C(═O)OC₁-C₄ alkyl, —CONR⁵R⁶, phenyl, —SO₂C₁-C₄alkyl, —SO₂NR⁵R⁶, cyano, oxo, hydroxyl, C₁-C₄alkoxy, C₃-C₇ cycloalkoxy, hydroxyC₁-C₄ alkyl-, C₁-C₄ alkoxy C₁-C₄ alkyl-, —OCF₃, —NR⁵R⁶, R⁵R⁶NC₁-C₄alkyl-, —NHC(O)C₁-C₄ alkyl, —NHCONR⁵R⁶, —NHSO₂C₁-C₄ alkyl, —NHSO₂NR⁵R⁶, and R⁹. In some embodiments, R² is selected from benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, 1-H-indazolyl, benzimidazolyl, dihydrobenzimidazolyl, benzoxazolyl, dihydrobenzoxazolyl, benzothiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzothiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, or pteridinyl, wherein said benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, 1-H-indazolyl, benzimidazolyl, dihydrobenzimidazolyl, benzoxazolyl, dihydrobenzoxazolyl, benzothiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzothiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl, all of which are optionally substituted with 1 to 3 substituents independently selected from halogen, C₁-C₄ alkyl, —CF₃, C₃-C₇ cycloalkyl, —C(O)C₁-C₄ alkyl, —C(O)C₃-C₇ cycloalkyl, —C(O)phenyl, —C₁-C₄(═O)OH, —C(═O)OC₁-C₄ alkyl, —C(O)NR⁵R⁶, phenyl, —SO₂C₁-C₄alkyl, —SO₂NR⁵R⁶, cyano, oxo, hydroxyl, C₁-C₄alkoxy, C₃-C₇ cycloalkoxy, hydroxyC₁-C₄ alkyl-, C₁-C₄ alkoxy C₁-C₄ alkyl-, —OCF₃, —NR⁵R⁶, R⁵R⁶NC₁-C₄ alkyl-, —NR⁶C(O)C₁-C₄ alkyl, —NR⁶C(O)NR⁵R⁶, —NR⁶SO₂C₁-C₄ alkyl, —NR⁶SO₂NR⁵R⁶, and R⁹. In some embodiments, R² is selected from phenyl and quinolinyl.

In some embodiments, the compound is 5-{[(3R)-1-(cyclopropylcarbonyl)-3-pyrrolidinyl]methyl}-6-[4-(1H-indol-5-yl)phenyl]-1-methyl-1,5-dihydro-4H-pyrazolo[3,4-d]pyrimidin-4-one; N-[4′-(5-{[(3R)-1-(cyclopropylcarbonyl)-3-pyrrolidinyl]methyl}-1-methyl-4-oxo-4,5-dihydro-1H-pyrazolo[3,4-d]pyrimidin-6-yl)-3-biphenylyl]-N,N-dimethylsulfamide; 5-{[(3R)-1-(cyclopropylcarbonyl)-3-pyrrolidinyl]methyl}-6-[4-(1H-indol-6-yl)phenyl]-1-methyl-1,5-dihydro-4H-pyrazolo[3,4-d]pyrimidin-4-one; 6-[4-(1-benzofuran-5-yl)phenyl]-5-{[(3R)-1-(cyclopropylcarbonyl)-3-pyrrolidinyl]methyl}-1-methyl-1,5-dihydro-4H-pyrazolo[3,4-d]pyrimidin-4-one; 5-{[(3R)-1-(cyclopropylcarbonyl)-3-pyrrolidinyl]methyl}-6-[4-(1H-indazol-5-yl)phenyl]-1-methyl-1,5-dihydro-4H-pyrazolo[3,4-d]pyrimidin-4-one; 5-{[(3R)-1-(cyclopropylcarbonyl)-3-pyrrolidinyl]methyl}-1-methyl-6-[4-(6-quinolinyl)phenyl]-1,5-dihydro-4H-pyrazolo[3,4-d]pyrimidin-4-one; 5-{[(3R)-1-(cyclopropylcarbonyl)-3-pyrrolidinyl]methyl}-1-methyl-6-[4-(7-quinolinyl)phenyl]-1,5-dihydro-4H-pyrazolo[3,4-d]pyrimidin-4-one; 6-[4-(1,3-benzothiazol-5-yl)phenyl]-5-{[(3R)-1-(cyclopropylcarbonyl)-3-pyrrolidinyl]methyl}-1-methyl-1,5-dihydro-4H-pyrazolo[3,4-d]pyrimidin-4-one; 5-[4-(1-benzofuran-5-yl)phenyl]-6-{[(3R)-1-(cyclopropylcarbonyl)-3-pyrrolidinyl]methyl}-2-methyl[1,3]oxazolo[5,4-d]pyrimidin-7(6H)-one; 4′-(6-{[(3R)-1-(cyclopropylcarbonyl)-3-pyrrolidinyl]methyl}-2-methyl-7-oxo-6,7-dihydro[1,3]oxazolo[5,4-d]pyrimidin-5-yl)-4-biphenylcarbonitrile; 6-{[(3R)-1-(cyclopropylcarbonyl)-3-pyrrolidinyl]methyl}-5-[4-(1H-indazol-5-yl)phenyl]-2-methyl[1,3]oxazolo[5,4-d]pyrimidin-7(6H)-one; 5-[4-(1,3-benzothiazol-5-yl)phenyl]-6-{[(3R)-1-(cyclopropylcarbonyl)-3-pyrrolidinyl]methyl}-2-methyl[1,3]oxazolo[5,4-d]pyrimidin-7(6H)-one; 6-{[(3R)-1-(cyclopropylcarbonyl)-3-pyrrolidinyl]methyl}-5-[4-(1H-indol-5-yl)phenyl]-2-methyl[1,3]oxazolo[5,4-d]pyrimidin-7(6H)-one; 6-{[(3S)-1-(cyclopropylcarbonyl)-3-pyrrolidinyl]methyl}-5-[4-(1H-indol-6-yl)phenyl]-1-methyl-1,5-dihydro-4H-pyrazolo[3,4-d]pyrimidin-4-one; 6-{[(3S)-1-(cyclopropylcarbonyl)-3-pyrrolidinyl]methyl}-5-[4-(1H-indol-5-yl)phenyl]-1-methyl-1,5-dihydro-4H-pyrazolo[3,4-d]pyrimidin-4-one; 6-[4-(1-benzofuran-5-yl)-2-fluorophenyl]-5-{[(3R)-1-(cyclopropylcarbonyl)-3-pyrrolidinyl]methyl}-1-methyl-1,5-dihydro-4H-pyrazolo[3,4-d]pyrimidin-4-one; 5-{[(3R)-1-(cyclopropylcarbonyl)-3-pyrrolidinyl]methyl}-6-(3,4′-difluoro-4-biphenylyl)-1-methyl-1,5-dihydro-4H-pyrazolo[3,4-d]pyrimidin-4-one; 5-{[(3R)-1-(cyclopropylcarbonyl)-3-pyrrolidinyl]methyl}-6-[2-fluoro-4-(1H-indol-5-yl)phenyl]-1-methyl-1,5-dihydro-4H-pyrazolo[3,4-d]pyrimidin-4-one; 5-{[(3R)-1-(cyclopropylcarbonyl)-3-pyrrolidinyl]methyl}-6-[2-fluoro-4-(1H-indol-6-yl)phenyl]-1-methyl-1,5-dihydro-4H-pyrazolo[3,4-d]pyrimidin-4-one; 2-[4-(1-benzofuran-5-yl)phenyl]-3-{[(3R)-1-(cyclopropylcarbonyl)-3-pyrrolidinyl]methyl}-4(3H)-quinazolinone; 3-{[(3R)-1-(cyclopropylcarbonyl)-3-pyrrolidinyl]methyl}-2-[4-(1H-indol-6-yl)phenyl]-4(3H)-quinazolinone; 3-{[(3R)-1-(cyclopropylcarbonyl)-3-pyrrolidinyl]methyl}-2-[4-(1H-indol-5-yl)phenyl]-4(3H)-quinazolinone; 3-{[(3R)-1-(cyclopropylcarbonyl)-3-pyrrolidinyl]methyl}-2-[4′-(methyloxy)-4-biphenylyl]-4(3H)-quinazolinone; 2-[2-chloro-4(methoxy)-4-biphenyl]-3-{[(3R)-1-(cyclopropylcarbonyl)-3-pyrrolidinyl]methyl}-4(3H)-quinazolinone; 2-[4-(1-benzofuran-5-yl)phenyl]-3-{[(3R)-1-(cyclopropylearbonyl)-3-pyrrolidinyl]methyl}-6-methyl-4(3H)-pyrimidinone; 3-{[(3R)-1-(cyclopropylcarbonyl)-3-pyrrolidinyl]methyl}-2-[4-(1H-indol-5-yl)phenyl-6-methyl-4(3H)-pyrimidinone; 3-{[(3R)-1-(cyclopropylcarbonyl)-3-pyrrolidinyl]methyl}-2-[4-(1H-indol-6-yl)phenyl-6-methyl-4(3H)-pyrimidinone; 4-({[(3R)-1-(cyclopropylcarbonyl)-3-pyrrolidinyl]methyl}oxy)-6-methyl-2-[4′-(methyloxy)-4-biphenylyl]pyrimidine; 2-[2′-chloro-4′-(methyloxy)-4-biphenylyl]-3-{[(3R)-1-(cyclopropylcarbonyl)-3-pyrrolidinyl]methyl}-6-methyl-4(3H)-pyrimidinone; N″-[4′-(1-{[(3R)-1-(cyclopropylcarbonyl)-3-pyrrolidinyl]methyl}-4-methyl-6-oxo-1,6-dihydro-2-pyrimidinyl)-3-biphenylyl]-N,N-dimethylsulfamide; 3-{[(3R)-1-(cyclopropylcarbonyl)-3-pyrrolidinyl]methyl}-2-(4′-fluoro-4-biphenylyl)-6-methyl-4(3H)-pyrimidinone; 2-[4-(1-benzofuran-5-yl)-2-fluorophenyl]-3-{[(3R)-1-(cyclopropylcarbonyl)-3-pyrrolidinyl]methyl}-6-methyl-4(3H)-pyrimidinone; 3-{[(3R)-1-(cyclopropylcarbonyl)-3-pyrrolidinyl]methyl}-2-[2-fluoro-4-(1H-indol-5-yl)phenyl]-6-methyl-4(3H)-pyrimidinone; 3-{[(3R)-1-(cyclopropylcarbonyl)-3-pyrrolidinyl]methyl}-2-[2-fluoro-4-(1H-indol-6-yl)phenyl]-6-methyl-4(3H)-pyrimidinone; 3-{[(3R)-1-(cyclopropylcarbonyl)-3-pyrrolidinyl]methyl}-2-(3,4′-difluoro-4-biphenylyl)-6-methyl-4(3H)-pyrimidinone; 3-{[(3R)-1-(cyclopropylcarbonyl)-3-pyrrolidinyl]methyl}-5,6-dimethyl-2-[4′-(methyloxy)-4-biphenylyl]-4(3H)-pyrimidinone; 2-[4-(1-benzofuran-5-yl)phenyl]-3-{[(3R)-1-(cyclopropylcarbonyl)-3-pyrrolidinyl]methyl}-5,6-dimethyl-4(3H)-pyrimidinone; 3-{[(3R)-1-(cyclopropylcarbonyl)-3-pyrrolidinyl]methyl}-2-[4-(1H-indol-6-yl)phenyl]-5,6-dimethyl-4(3H)-pyrimidinone; 3-{[(3R)-1-(cyclopropylcarbonyl)-3-pyrrolidinyl]methyl}-2-(4′-fluoro-4-biphenylyl)-5,6-dimethyl-4(3H)-pyrimidinone; 3-{[(3R)-1-(cyclopropylcarbonyl)-3-pyrrolidinyl]methyl}-2-[2-fluoro-4-(1H-indol-6-yl)phenyl]-5,6-dimethyl-4(3H)-pyrimidinone; 2-[4-(1-benzofuran-5-yl)-2-fluorophenyl]-3-{[(3R)-1-(cyclopropylcarbonyl)-3-pyrrolidinyl]methyl}-5,6-dimethyl-4(3H)-pyrimidinone; 3-{[(3R)-1-(cyclopropylcarbonyl)-3-pyrrolidinyl]methyl}-2-(3,4′-difluoro-4-biphenylyl)-5,6-dimethyl-4(3H)-pyrimidinone; 3-{[(3R)-1-(cyclopropylcarbonyl)-3-pyrrolidinyl]methyl}-5-ethyl-2-[4-(1H-indol-6-yl)phenyl]-6-methyl-4(3H)-pyrimidinone; 2-[4-(1-benzofuran-5-yl)phenyl]-3-{[(3R)-1-(cyclopropylcarbonyl)-3-pyrrolidinyl]methyl}-5-ethyl-6-methyl-4(3H)-pyrimidinone; 3-{[(3R)-1-(cyclopropylcarbonyl)-3-pyrrolidinyl]methyl}-5-ethyl-2-[4-(1H-indol-5-yl)phenyl]-6-methyl-4(3H)-pyrimidinone; 3-{[(3R)-1-(cyclopropylcarbonyl)-3-pyrrolidinyl]methyl}-5-ethyl-2-(4′-fluoro-4-biphenylyl)-6-methyl-4(3H)-pyrimidinone; 2-{[(3S)-1-(cyclopropylcarbonyl)-3-pyrrolidinyl]methyl}-1-[4-(1H-indol-6-yl)phenyl]-6-methyl-4(3H)-pyrimidinone; 2-{[(3S)-1-(cyclopropylcarbonyl)-3-pyrrolidinyl]methyl}-1-[4-(1H-indol-6-yl)phenyl]-6-methyl-4(1H)-pyrimidinone; or 1-[4-(1-benzofuran-5-yl)phenyl]-2-{[(3S)-1-(cyclopropylcarbonyl)-3-pyrrolidinyl]methyl}-6-methyl-4(1H)-pyrimidinone, or a pharmaceutically acceptable salt thereof.

In some embodiments, the compound is one of the following:

| | Compound |
|---|---|
| 1132 | 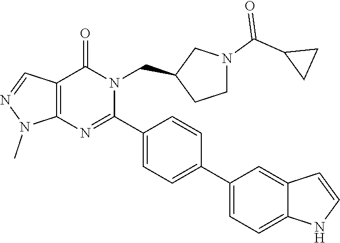 [SMILES: Cn1ncc2c(=O)n(C[C@@H]3CCN(C(=O)C4CC4)C3)c(-c3ccc(-c4ccc5[nH]ccc5c4)cc3)nc21] |
| 1133 | 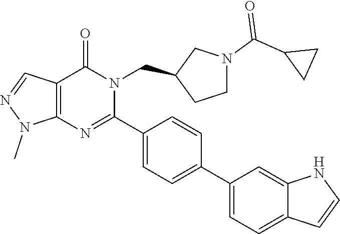 [SMILES: Cn1ncc2c(=O)n(C[C@@H]3CCN(C(=O)C4CC4)C3)c(-c3ccc(-c4ccc5cc[nH]c5c4)cc3)nc21] |
| 1134 | 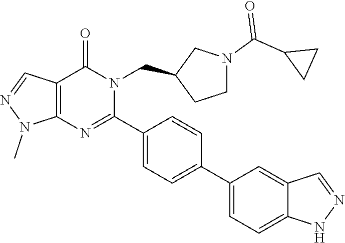 [SMILES: Cn1ncc2c(=O)n(C[C@@H]3CCN(C(=O)C4CC4)C3)c(-c3ccc(-c4ccc5[nH]ncc5c4)cc3)nc21] |
| 1135 | 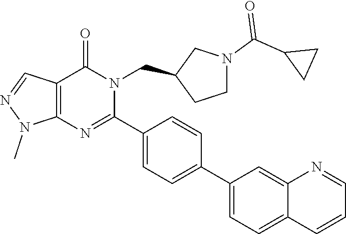 [SMILES: Cn1ncc2c(=O)n(C[C@@H]3CCN(C(=O)C4CC4)C3)c(-c3ccc(-c4ccc5cccnc5c4)cc3)nc21] |
| 1136 | 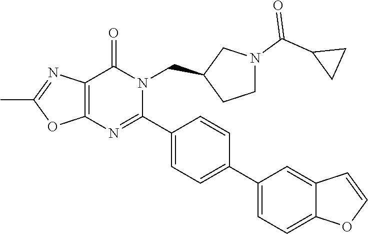 [SMILES: Cc1nc2c(=O)n(C[C@@H]3CCN(C(=O)C4CC4)C3)c(-c3ccc(-c4ccc5occc5c4)cc3)nc2o1] |
| 1137 | 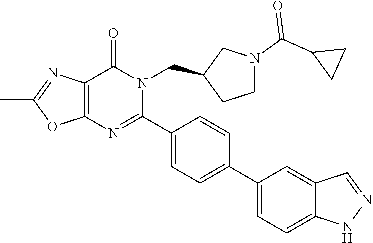 [SMILES: Cc1nc2c(=O)n(C[C@@H]3CCN(C(=O)C4CC4)C3)c(-c3ccc(-c4ccc5[nH]ncc5c4)cc3)nc2o1] |
| 1138 | 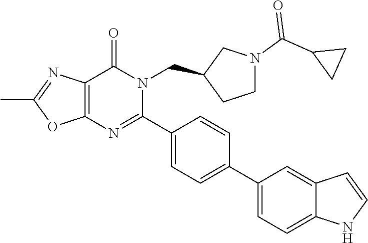 [SMILES: Cc1nc2c(=O)n(C[C@@H]3CCN(C(=O)C4CC4)C3)c(-c3ccc(-c4ccc5[nH]ccc5c4)cc3)nc2o1] |
| 1139 | 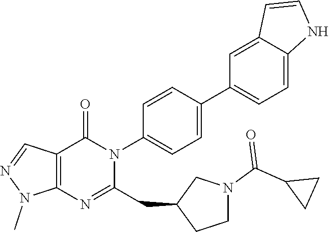 [SMILES: Cn1ncc2c(=O)n(-c3ccc(-c4ccc5[nH]ccc5c4)cc3)c(C[C@@H]3CCN(C(=O)C4CC4)C3)nc21] |
| 1140 | 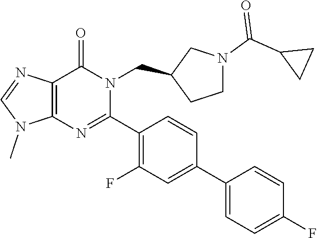 [SMILES: Cn1cnc2c(=O)n(C[C@@H]3CCN(C(=O)C4CC4)C3)c(-c3ccc(-c4ccc(F)cc4)cc3F)nc21] |
| 1141 | 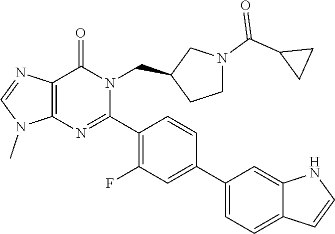 [SMILES: Cn1cnc2c(=O)n(C[C@@H]3CCN(C(=O)C4CC4)C3)c(-c3ccc(-c4ccc5cc[nH]c5c4)cc3F)nc21] |
| 1142 | 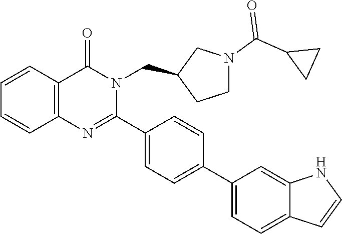 [SMILES: O=C(C1CC1)N1CC[C@@H](Cn2c(-c3ccc(-c4ccc5cc[nH]c5c4)cc3)nc3ccccc3c2=O)C1] |
| 1143 | 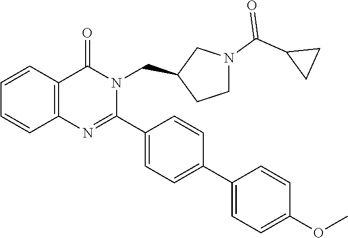 [SMILES: COc1ccc(-c2ccc(-c3nc4ccccc4c(=O)n3C[C@@H]3CCN(C(=O)C4CC4)C3)cc2)cc1] |
| 1144 | 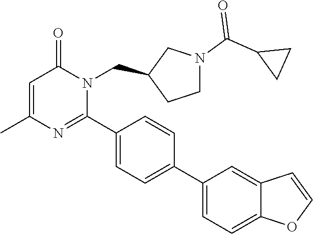 [SMILES: Cc1cc(=O)n(C[C@@H]2CCN(C(=O)C3CC3)C2)c(-c2ccc(-c3ccc4occc4c3)cc2)n1] |
| 1145 | 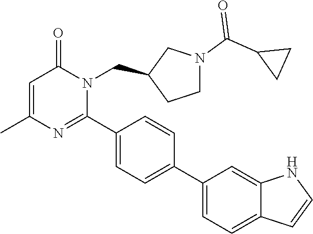 [SMILES: Cc1cc(=O)n(C[C@@H]2CCN(C(=O)C3CC3)C2)c(-c2ccc(-c3ccc4cc[nH]c4c3)cc2)n1] |
| 1146 | 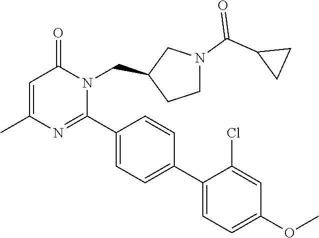 |
| 1147 | 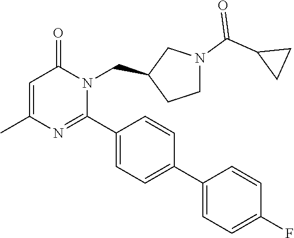 [SMILES: Cc1cc(=O)n(C[C@@H]2CCN(C(=O)C3CC3)C2)c(-c2ccc(-c3ccc(F)cc3)cc2)n1] |
| 1148 | 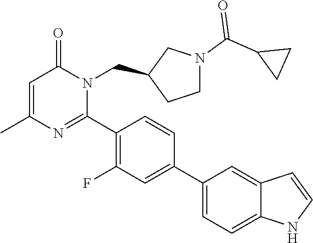 [SMILES: Cc1cc(=O)n(C[C@@H]2CCN(C(=O)C3CC3)C2)c(-c2ccc(-c3ccc4[nH]ccc4c3)cc2F)n1] |
| 1149 | 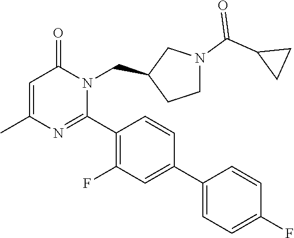 [SMILES: Cc1cc(=O)n(C[C@@H]2CCN(C(=O)C3CC3)C2)c(-c2ccc(-c3ccc(F)cc3)cc2F)n1] |
| 1150 | 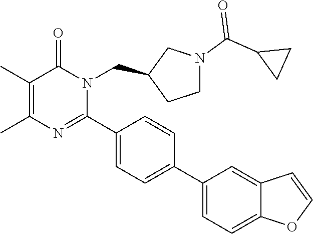 [SMILES: Cc1nc(-c2ccc(-c3ccc4occc4c3)cc2)n(C[C@@H]2CCN(C(=O)C3CC3)C2)c(=O)c1C] |
| 1151 | 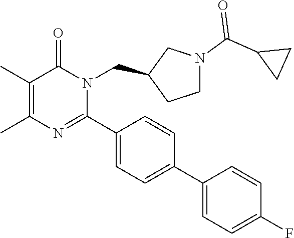 [SMILES: Cc1nc(-c2ccc(-c3ccc(F)cc3)cc2)n(C[C@@H]2CCN(C(=O)C3CC3)C2)c(=O)c1C] |
| 1152 | 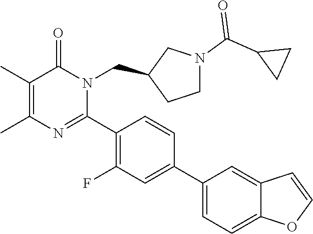 [SMILES: Cc1nc(-c2ccc(-c3ccc4occc4c3)cc2F)n(C[C@@H]2CCN(C(=O)C3CC3)C2)c(=O)c1C] |
| 1153 | 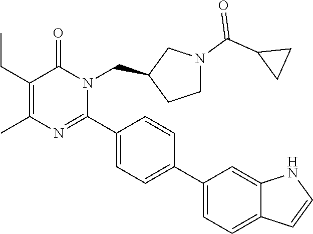 [SMILES: CCc1c(C)nc(-c2ccc(-c3ccc4cc[nH]c4c3)cc2)n(C[C@@H]2CCN(C(=O)C3CC3)C2)c1=O] |
| 1154 | 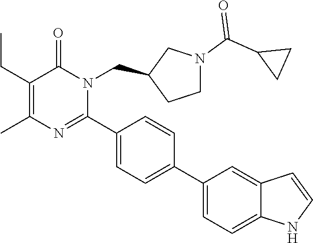 [SMILES: CCc1c(C)nc(-c2ccc(-c3ccc4[nH]ccc4c3)cc2)n(C[C@@H]2CCN(C(=O)C3CC3)C2)c1=O] |
| 1155 | 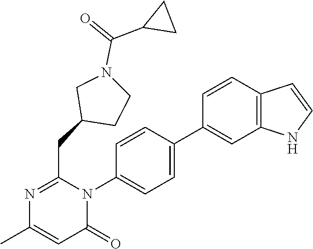 [SMILES: Cc1cc(=O)n(-c2ccc(-c3ccc4cc[nH]c4c3)cc2)c(C[C@@H]2CCN(C(=O)C3CC3)C2)n1] |
| 1156 | 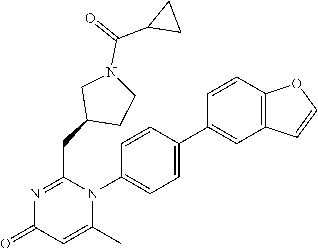 [SMILES: Cc1cc(=O)nc(C[C@@H]2CCN(C(=O)C3CC3)C2)n1-c1ccc(-c2ccc3occc3c2)cc1] |
| 1157 | 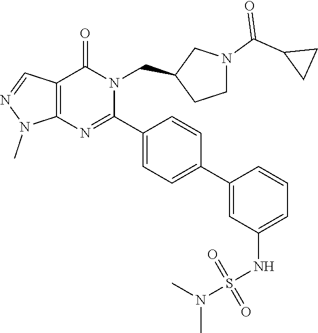 [SMILES: CN(C)S(=O)(=O)Nc1cccc(-c2ccc(-c3nc4c(cnn4C)c(=O)n3C[C@@H]3CCN(C(=O)C4CC4)C3)cc2)c1] |
| 1158 | 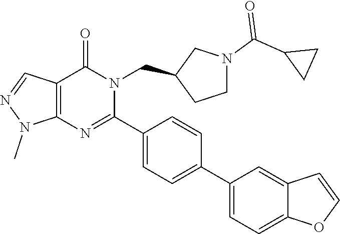 [SMILES: Cn1ncc2c(=O)n(C[C@@H]3CCN(C(=O)C4CC4)C3)c(-c3ccc(-c4ccc5occc5c4)cc3)nc21] |
| 1159 | 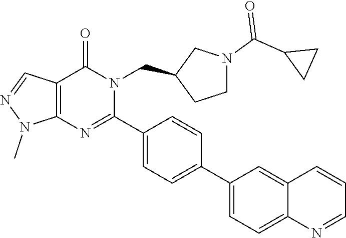 [SMILES: Cn1ncc2c(=O)n(C[C@@H]3CCN(C(=O)C4CC4)C3)c(-c3ccc(-c4ccc5ncccc5c4)cc3)nc21] |
| 1160 | 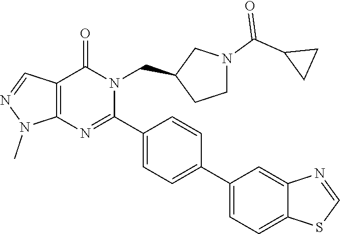 [SMILES: Cn1ncc2c(=O)n(C[C@@H]3CCN(C(=O)C4CC4)C3)c(-c3ccc(-c4ccc5scnc5c4)cc3)nc21] |
| 1161 | 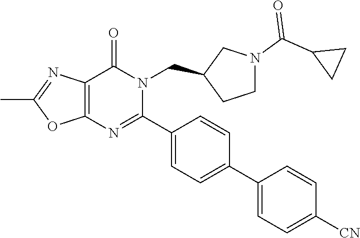 [SMILES: Cc1nc2c(=O)n(C[C@@H]3CCN(C(=O)C4CC4)C3)c(-c3ccc(-c4ccc(C#N)cc4)cc3)nc2o1] |
| 1162 | 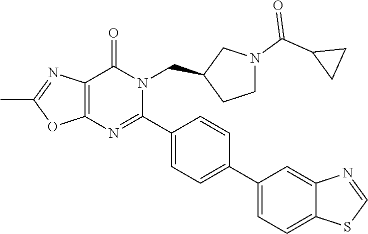 [SMILES: Cc1nc2c(=O)n(C[C@@H]3CCN(C(=O)C4CC4)C3)c(-c3ccc(-c4ccc5scnc5c4)cc3)nc2o1] |
| 1163 | 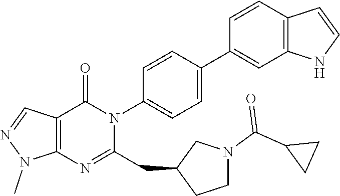 [SMILES: Cn1ncc2c(=O)n(-c3ccc(-c4ccc5cc[nH]c5c4)cc3)c(C[C@@H]3CCN(C(=O)C4CC4)C3)nc21] |
| 1164 | 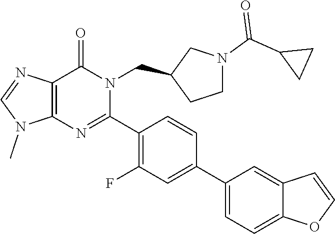 [SMILES: Cn1cnc2c(=O)n(C[C@@H]3CCN(C(=O)C4CC4)C3)c(-c3ccc(-c4ccc5occc5c4)cc3F)nc21] |
| 1165 | 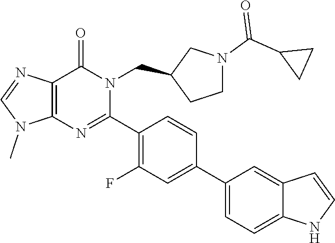 [SMILES: Cn1cnc2c(=O)n(C[C@@H]3CCN(C(=O)C4CC4)C3)c(-c3ccc(-c4ccc5[nH]ccc5c4)cc3F)nc21] |
| 1166 | 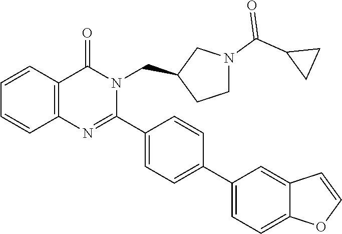 [SMILES: O=C(C1CC1)N1CC[C@@H](Cn2c(-c3ccc(-c4ccc5occc5c4)cc3)nc3ccccc3c2=O)C1] |
| 1167 | 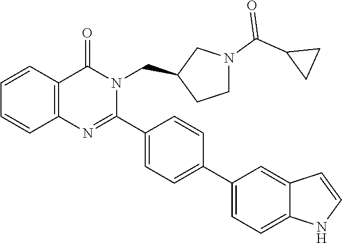 [SMILES: O=C(C1CC1)N1CC[C@@H](Cn2c(-c3ccc(-c4ccc5[nH]ccc5c4)cc3)nc3ccccc3c2=O)C1] |
| 1168 | 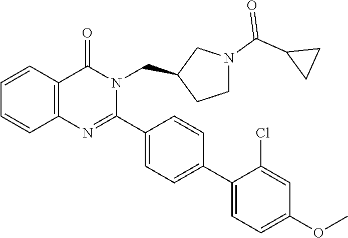 [SMILES: COc1ccc(-c2ccc(-c3nc4ccccc4c(=O)n3C[C@@H]3CCN(C(=O)C4CC4)C3)cc2)c(Cl)c1] |
| 1169 | 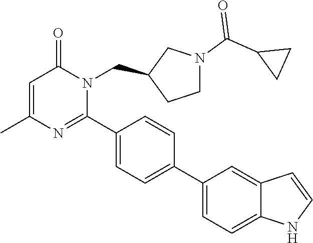 [SMILES: Cc1cc(=O)n(C[C@@H]2CCN(C(=O)C3CC3)C2)c(-c2ccc(-c3ccc4[nH]ccc4c3)cc2)n1] |
| 1170 | 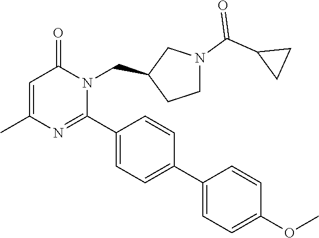 |
| 1171 | 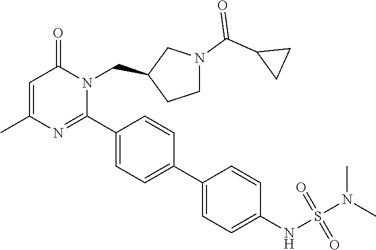 |
| 1172 | 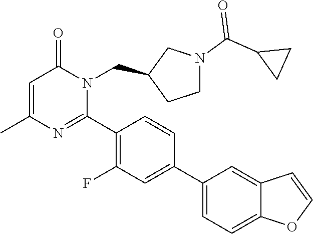 [SMILES: Cc1cc(=O)n(C[C@@H]2CCN(C(=O)C3CC3)C2)c(-c2ccc(-c3ccc4occc4c3)cc2F)n1] |
| 1173 | 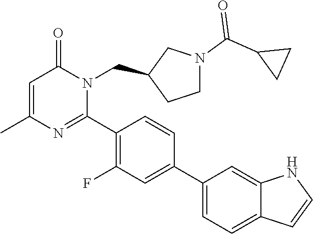 [SMILES: Cc1cc(=O)n(C[C@@H]2CCN(C(=O)C3CC3)C2)c(-c2ccc(-c3ccc4cc[nH]c4c3)cc2F)n1] |
| 1174 | 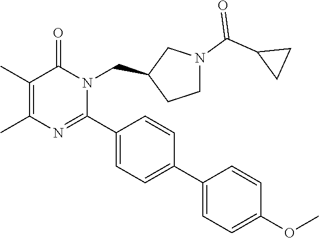 |
| 1175 | 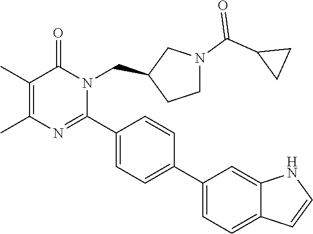 [SMILES: Cc1nc(-c2ccc(-c3ccc4cc[nH]c4c3)cc2)n(C[C@@H]2CCN(C(=O)C3CC3)C2)c(=O)c1C] |
| 1176 | 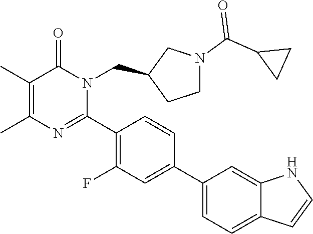 [SMILES: Cc1nc(-c2ccc(-c3ccc4cc[nH]c4c3)cc2F)n(C[C@@H]2CCN(C(=O)C3CC3)C2)c(=O)c1C] |
| 1177 | 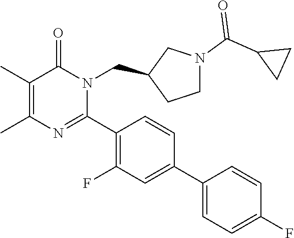 [SMILES: Cc1nc(-c2ccc(-c3ccc(F)cc3)cc2F)n(C[C@@H]2CCN(C(=O)C3CC3)C2)c(=O)c1C] |
| 1178 | 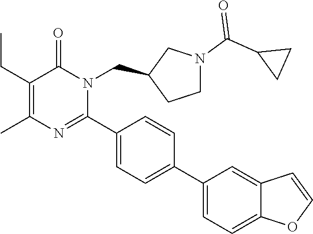 [SMILES: CCc1c(C)nc(-c2ccc(-c3ccc4occc4c3)cc2)n(C[C@@H]2CCN(C(=O)C3CC3)C2)c1=O] |
| 1179 | 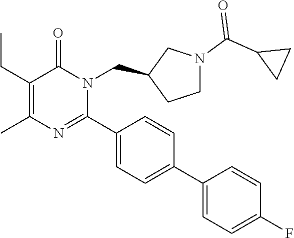 [SMILES: CCc1c(C)nc(-c2ccc(-c3ccc(F)cc3)cc2)n(C[C@@H]2CCN(C(=O)C3CC3)C2)c1=O] |
| 1180 | 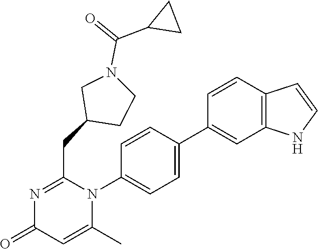 [SMILES: Cc1cc(=O)nc(C[C@@H]2CCN(C(=O)C3CC3)C2)n1-c1ccc(-c2ccc3cc[nH]c3c2)cc1] |
| 1181 | |

In some embodiments, the compound has the structure of formula (XXIV):

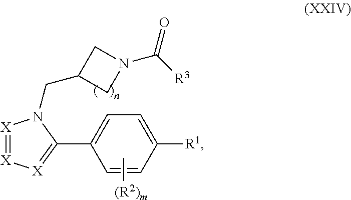

wherein: R¹ is phenyl, naphthyl, 5- or 6-membered heteroaryl, or 9- or 10-membered heterocyclyl; wherein said phenyl, naphthyl, 5- or 6-membered heteroaryl, or 9- or 10-membered heterocyclyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹; when present each R² is independently selected from the group consisting of halogen, (C₁-C₆)alkyl, hydroxyl, and (C₁-C₄)alkoxy; R³ is selected from the group consisting of (C₁-C₆)alkyl, —CF₃, (C₃-C₇)cycloalkyl, (C₁-C₄)alkoxy, and —NR⁷R⁸; wherein said (C₁-C₆)alkyl is optionally substituted by hydroxyl, (C₁-C₄)alkoxy, —CF₃, or cyano, and wherein said (C₃-C₇)cycloalkyl is optionally substituted 1 or 2 times independently by halogen, (C₁-C₄)alkyl, hydroxy(C₁-C₄)alkyl-, (C₁-C₄)alkoxy(C₁-C₄)alkyl-, —CF₃, or cyano; each X is independently N or CR⁴, wherein at least one X is N; when present each R₄ is independently hydrogen or (C₁-C₄)alkyl; R⁵ is selected from the group consisting of hydrogen, (C₁-C₄)alkyl, (C₃-C₇)cycloalkyl, phenyl, and phenyl(C₁-C₃)alkyl-; R⁶ is hydrogen, (C₁-C₄)alkyl, or (C₃-C₇)cycloalkyl; or R⁵ and R⁶ taken together with the nitrogen to which they are attached represent a 3- to 7-membered saturated ring optionally containing one other heteroatom which is oxygen, nitrogen, or sulfur, which ring is optionally substituted 1 or 2 times independently by oxo or (C₁-C₄)alkyl; R⁷ and R⁸ are each independently hydrogen, (C₁-C₄)alkyl, or (C₃-C₇)cycloalkyl; or R⁷ and R⁸ taken together with the nitrogen to which they are attached represent a 3- to 7-membered saturated ring optionally containing one other heteroatom which is oxygen, nitrogen, or sulfur, which ring is optionally substituted 1 or 2 times independently by oxo or (C₁-C₄)alkyl; R⁹ is a 5-membered heteroaryl ring containing 1 to 4 heteroatoms selected from oxygen, nitrogen, and sulfur, or a 6-membered heteroaryl ring containing 1 to 3 nitrogen atoms, which 5- or 6-membered ring is optionally substituted 1 or 2 times independently by halogen, (C₁-C₄)alkyl, —CF₃, (C₁-C₄)alkoxy, or —NR⁵R⁶; m is 0-3; and n is 1 or 2; or pharmaceutically acceptable salts thereof.

In some embodiments, the compound has the structure of Formula (XXIV-A):

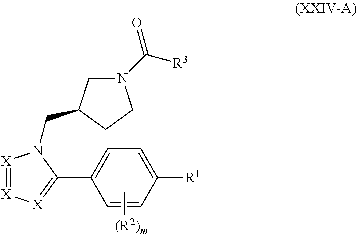

or a pharmaceutically acceptable salt thereof.

In some embodiments, the compound has the structure of Formula (XXIV-B):

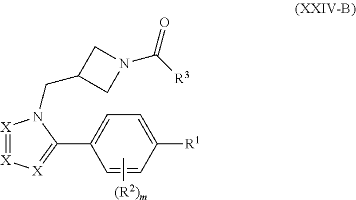

or a pharmaceutically acceptable salt thereof.

In one embodiment, R¹ is phenyl which is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹, or pharmaceutically acceptable salts thereof. In another embodiment R¹ is phenyl, 4-fluorophenyl, 3-chlorophenyl, 4-chlorophenyl, 2,4-difluorophenyl, 3,4-difluorophenyl, 3-chloro-4-fluorophenyl, 2,4-dichlorophenyl, 2-fluoro-4-methylphenyl, 3-fluoro-4-methylphenyl, 4-fluoro-3-methylphenyl, 2-fluoro-4-methoxyphenyl, 3-fluoro-4-methoxyphenyl, 4-fluoro-3-hydroxyphenyl, 4-fluoro-3-methoxyphenyl, 2-chloro-4-methoxyphenyl, 3-chloro-4-methoxyphenyl, 3-methylphenyl, 4-methylphenyl, 2,4-dimethylphenyl, 2-cyanophenyl, 4-cyanophenyl, 3-hydroxyphenyl, 4-hydroxyphenyl, 4-methoxyphenyl, 4-ethoxyphenyl, 3-hydroxy-4-methylphenyl, 3-methoxy-4-methylphenyl, 4-methoxy-3-methylphenyl, 3-hydroxy-4-methoxyphenyl, 4-(dimethylamino)phenyl, 3-{[(dimethylamino)sulfonyl]amino}phenyl, 4-(1H-pyrazol-1-yl)phenyl, 4-(1H-pyrazol-5-yl)phenyl, or 3-(1H-tetrazol-5-yl)phenyl, or pharmaceutically acceptable salts thereof. In another embodiment, R¹ is 5- or 6-membered heteroaryl which is optionally substituted 1 to 3 times independently by halogen, (C₁-C₄)alkyl, —CF₃, (C₃-C₇)cycloalkyl, —CO(C₁-C₄)alkyl, —CO(C₃-C₇) cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹, or pharmaceutically acceptable salts thereof. In another embodiment, R¹ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, wherein said furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl is optionally substituted 1 to 3 times independently by halogen, (C₁-C₄)alkyl, —CF₃, (C₃-C₇)cycloalkyl, —CO(C₁-C₄)alkyl, —CO(C₃-C₇) cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹, or pharmaceutically acceptable salts thereof. In another embodiment, R¹ is pyridin-3-yl, or pharmaceutically acceptable salts thereof. In another embodiment, R¹ is 9- or 10-membered heterocyclyl which is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹, or pharmaceutically acceptable salts thereof. In another embodiment, R¹ is benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, or pteridinyl, wherein said benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, or pteridinyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹, or pharmaceutically acceptable salts thereof. In another embodiment, R¹ is benzofuranyl, 2,3-dihydrobenzofuryl, indolyl, indolinyl, benzthiazolyl, benzimidazolyl, benzoxazolyl, indazolyl, pyrrolopyridinyl, imidazopyridinyl, quinolinyl, or isoquinolinyl, wherein said benzofuranyl, 2,3-dihydrobenzofuryl, indolyl, indolinyl, benzthiazolyl, benzimidazolyl, benzoxazolyl, indazolyl, pyrrolopyridinyl, imidazopyridinyl, quinolinyl, or isoquinolinyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹, or pharmaceutically acceptable salts thereof. In another embodiment, R¹ is benzofuranyl, 2,3-dihydrobenzofuryl, indolyl, indolinyl, benzthiazolyl, indazolyl, pyrrolopyridinyl, imidazopyridinyl, or quinolinyl, wherein said benzofuranyl, 2,3-dihydrobenzofuryl, indolyl, indolinyl, benzthiazolyl, indazolyl, pyrrolopyridinyl, imidazopyridinyl, or quinolinyl is optionally substituted by (C₁-C₄)alkyl, —CF₃, cyano, hydroxyl, methoxy, —OCF₃, amino, methylamino or dimethylamino, or pharmaceutically acceptable salts thereof. In another embodiment, R¹ is benzofuran-5-yl, 2,3-dihydro-1-benzofuran-5-yl, 1H-indol-4-yl, 1H-indol-5-yl, 1H-indol-6-yl, 1-methyl-1H-indole-5-yl, 1H-indazol-4-yl, 1H-indazol-5-yl, 1H-indazol-6-yl, 2,3-dihydro-1H-indol-5-yl, 1,3-benzothiazol-6-yl, imidazo[1,2-a]pyridin-7-yl, 1H-pyrrolo[3,2-b]pyridin-6-yl, 1-methyl-1H-pyrrolo[2,3-b]pyridin-5-yl, quinolin-3-yl, quinolin-6-yl, or quinolin-7-yl, or pharmaceutically acceptable salts thereof. In another embodiment, R² is fluoro, chloro, hydroxyl, methoxy, or methyl, and m is 1, or pharmaceutically acceptable salts thereof. In another embodiment R³ is (C₁-C₄)alkyl, —CF₃, (C₃-C₆)cycloalkyl, methoxy, or dimethylamino, wherein said (C₃-C₆)cycloalkyl is optionally substituted 1 or 2 times independently by fluoro or methyl, or pharmaceutically acceptable salts thereof. In another embodiment, R³ is methyl, ethyl, isopropyl, t-butyl, —CF₃, cyclopropyl, 1-methyl-cyclopropyl, 2,2-difluoro-cyclopropyl, cyclopentyl, methoxy, or dimethylamino, or pharmaceutically acceptable salts thereof. In another embodiment, R³ is cyclopropyl, or pharmaceutically acceptable salts thereof. In another embodiment, R₄ is hydrogen or methyl, or pharmaceutically acceptable salts thereof. One particular embodiment of the invention is a compound of Formula (XXIV) wherein: R¹ is phenyl, 5- or 6-membered heteroaryl, or 9- or 10-membered heterocyclyl; wherein said phenyl, 5- or 6-membered heteroaryl, or 9- or 10-membered heterocyclyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹; when present each R² is independently selected from the group consisting of halogen, (C₁-C₆)alkyl, hydroxyl, and (C₁-C₄)alkoxy; R³ is selected from the group consisting of (C₁-C₆)alkyl, —CF₃, (C₃-C₇)cycloalkyl, (C₁-C₄)alkoxy, and —NR⁷R⁸; wherein said (C₃-C₇) cycloalkyl is optionally substituted 1 or 2 times independently by halogen or (C₁-C₄)alkyl; each X is independently N or CR⁴, wherein at least one X is N; when present each R₄ is independently hydrogen or (C₁-C₄)alkyl; R⁵ is selected from the group consisting of hydrogen, (C₁-C₄)alkyl, phenyl, and phenyl(C₁-C₃)alkyl-; R⁶ is hydrogen or (C₁-C₄)alkyl; or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or (C₁-C₄)alkyl; R⁷ and R⁸ are each independently hydrogen or (C₁-C₄)alkyl; or R⁷ and R⁸ taken together with the nitrogen to which they are attached represent a 3- to 7-membered saturated ring optionally containing one other heteroatom which is oxygen, nitrogen, or sulfur; R⁹ is a 5-membered heteroaryl ring containing 1 to 4 heteroatoms selected from oxygen, nitrogen, and sulfur, which is optionally substituted 1 or 2 times independently by halogen, (C₁-C₄)alkyl, (C₁-C₄)alkoxy, or —NR⁵R⁶; m is 0-3; and n is 1 or 2; or pharmaceutically acceptable salts thereof. Another particular embodiment of the invention is a compound of Formula (XXIV)(A) wherein: R¹ is phenyl, 5- or 6-membered heteroaryl, or 9- or 10-membered heterocyclyl; wherein said phenyl, 5- or 6-membered heteroaryl, or 9- or 10-membered heterocyclyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹; when present each R² is independently selected from the group consisting of halogen, (C₁-C₆)alkyl, hydroxyl, and (C₁-C₄)alkoxy; R³ is selected from the group consisting of (C₁-C₆)alkyl, —CF₃, (C₃-C₇)cycloalkyl, (C₁-C₄)alkoxy, and —NR⁷R⁸; wherein said (C₃-C₇)cycloalkyl is optionally substituted 1 or 2 times independently by halogen or (C₁-C₄)alkyl; each X is independently N or CR⁴, wherein at least one X is N; when present each R₄ is independently hydrogen or (C₁-C₄)alkyl; R⁵ is selected from the group consisting of hydrogen, (C₁-C₄)alkyl, phenyl, and phenyl(C₁-C₃)alkyl-; R⁶ is hydrogen or (C₁-C₄)alkyl; or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or (C₁-C₄)alkyl; R⁷ and R⁸ are each independently hydrogen or (C₁-C₄)alkyl; or R⁷ and R⁸ taken together with the nitrogen to which they are attached represent a 3- to 7-membered saturated ring optionally containing one other heteroatom which is oxygen, nitrogen, or sulfur; R⁹ is a 5-membered heteroaryl ring containing 1 to 4 heteroatoms selected from oxygen, nitrogen, and sulfur, which is optionally substituted 1 or 2 times independently by halogen, (C₁-C₄)alkyl, (C₁-C₄)alkoxy, or —NR⁵R⁶; and m is 0-3; or pharmaceutically acceptable salts thereof. Another particular embodiment of the invention is a compound of Formula (XXIV)(B) wherein: R¹ is phenyl, 5- or 6-membered heteroaryl, or 9- or 10-membered heterocyclyl; wherein said phenyl, 5- or 6-membered heteroaryl, or 9- or 10-membered heterocyclyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹; when present each R² is independently selected from the group consisting of halogen, (C₁-C₆)alkyl, hydroxyl, and (C₁-C₄)alkoxy; R³ is selected from the group consisting of (C₁-C₆)alkyl, —CF₃, (C₃-C₇)cycloalkyl, (C₁-C₄)alkoxy, and —NR⁷R⁸; wherein said (C₃-C₇)cycloalkyl is optionally substituted 1 or 2 times independently by halogen or (C₁-C₄)alkyl; each X is independently N or CR⁴, wherein at least one X is N; when present each R₄ is independently hydrogen or (C₁-C₄)alkyl; R⁵ is selected from the group consisting of hydrogen, (C₁-C₄)alkyl, phenyl, and phenyl(C₁-C₃)alkyl-; R⁶ is hydrogen or (C₁-C₄)alkyl; or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or (C₁-C₄)alkyl; R⁷ and R⁸ are each independently hydrogen or (C₁-C₄)alkyl; or R⁷ and R⁸ taken together with the nitrogen to which they are attached represent a 3- to 7-membered saturated ring optionally containing one other heteroatom which is oxygen, nitrogen, or sulfur; R⁹ is a 5-membered heteroaryl ring containing 1 to 4 heteroatoms selected from oxygen, nitrogen, and sulfur, which is optionally substituted 1 or 2 times independently by halogen, (C₁-C₄)alkyl, (C₁-C₄)alkoxy, or —NR⁵R⁶; and m is 0-3; or pharmaceutically acceptable salts thereof.

In some embodiments, the compound is 6-[4-(1-{[(3R)-1-(cyclopropylcarbonyl)-3-pyrrolidinyl]methyl}-1H-tetrazol-5-yl)phenyl]-1H-indole; 5-[4-(1-benzofuran-5-yl)phenyl]-1-{[(3R)-1-(cyclopropylcarbonyl)-3-pyrrolidinyl]methyl}-1H-tetrazole; 5-[4-(1-{[(3R)-1-(cyclopropylcarbonyl)-3-pyrrolidinyl]methyl}-1H-tetrazol-5-yl)phenyl]-1H-indole; 1-{[(3R)-1-(cyclopropylcarbonyl)-3-pyrrol idinyl]methyl}-5-(2′,4′-dichloro-4-biphenylyl)-1H-tetrazole; 5-[2′-chloro-4′-(methyl oxy)-4-biphenylyl]-1-{[(3R)-1-(cyclopropylcarbonyl)-3-pyrrol idinyl]methyl}-1H-tetrazole; 1-{[(3R)-1-(cyclopropylcarbonyl)-3-pyrrolidinyl]methyl}-5-(4′-fluoro-4-biphenylyl)-1H-tetrazole; 6-[4-(1-{[(3R)-1-(cyclopropylcarbonyl)-3-pyrrolidinyl]methyl}-1H-tetrazol-5-yl)phenyl]-1H-indazole; 6-[4-(1-{[(3R)-1-(cyclopropylcarbonyl)-3-pyrrolidinyl]methyl}-1H-1,2,3-triazol-5-yl)phenyl]-1H-indole; 5-[4-(1-{[(3R)-1-(cyclopropylcarbonyl)-3-pyrrolidinyl]methyl}-1H-1,2,3-triazol-5-yl)phenyl]-1H-indole; 5-[4-(1-benzofuran-5-yl)phenyl]-1-{[(3R)-1-(cyclopropylcarbonyl)-3-pyrrolidinyl]methyl}-1H-1,2,3-triazole; 5-[4-(1-{[(3R)-1-(cyclopropylcarbonyl)-3-pyrrolidinyl]methyl}-1H-1,2,3-triazol-5-yl)phenyl]-1H-indazole; 1-{[(3R)-1-(cyclopropylcarbonyl)-3-pyrrolidinyl]methyl}-5-(2′,4′-dichloro-4-biphenylyl)-1H-1,2,3-triazole; 5-[2′-chloro-4′-(methyloxy)-4-biphenylyl]-1-{[(3R)-1-(cyclopropylcarbonyl)-3-pyrrolidinyl]methyl}-1H-1,2,3-triazole; 6-[4-(4-{[(3S)-1-(cyclopropylcarbonyl)-3-pyrrolidinyl]methyl}-4H-1,2,4-triazol-3-yl)phenyl]-1H-indole; 4-{[(3S)-1-(cyclopropylcarbonyl)-3-pyrrolidinyl]methyl}-3-(2′,4′-dichloro-4-biphenylyl)-5-methyl-4H-1,2,4-triazole; 6-[4-(4-{[(3S)-1-(cyclopropylcarbonyl)-3-pyrrolidinyl]methyl}-5-methyl-4H-1,2,4-triazol-3-yl)phenyl]-1H-indole; 3-[4-(1-benzofuran-5-yl)phenyl]-4-{[(3S)-1-(cyclopropylcarbonyl)-3-pyrrol idinyl]methyl}-5-methyl-4H-1,2,4-triazole; 4-{[(3S)-1-(cyclopropylcarbonyl)-3-pyrrolidinyl]methyl}-3-(2′,4′-dichloro-4-biphenylyl)-4H-1,2,4-triazole; 5-[4-(4-{[(3S)-1-(cyclopropylcarbonyl)-3-pyrrolidinyl]methyl}-4H-1,2,4-triazol-3-yl)phenyl]-1H-indazole; 3-[2′-chloro-4′-(methyloxy)-4-biphenylyl]-4-{[(3S)-1-(cyclopropylcarbonyl)-3-pyrrolidinyl]methyl}-4H-1,2,4-triazole; 5-[4-(1-{[(3S)-1-(cyclopropylcarbonyl)-3-pyrrolidinyl]methyl}-1H-imidazol-2-yl)phenyl]-1H-indole; 6-[4-(1-{[(3S)-1-(cyclopropylcarbonyl)-3-pyrrolidinyl]methyl}-1H-imidazol-2-yl)phenyl]-1H-indole; 2-(3′-chloro-4-biphenylyl)-1-{[(3S)-1-(cyclopropylcarbonyl)-3-pyrrolidinyl]methyl}-1H-imidazole; 1-{[1-(cyclopropylcarbonyl)-3-pyrrolidinyl]methyl}-2-[4′-(methyloxy)-4-biphenylyl]-1H-imidazole; 1-{[1-(cyclopropylcarbonyl)-3-pyrrolidinyl]methyl}-2-(3′-fluoro-4′-methyl-4-biphenylyl)-1H-imidazole; 2-4 biphenylyl)-1-{[(3S)-1-(cyclopropylcarbonyl)-3-pyrrolidinyl]methyl}-1H-imidazole; 5-[4-(1-{[1-(cyclopropylcarbonyl)-3-pyrrolidinyl]methyl}-1H-imidazol-2-yl)phenyl]-1H-indole; 2-(3′-chloro-4-biphenylyl)-1-{[1-(cyclopropylcarbonyl)-3-pyrrolidinyl]methyl}-1H-imidazole; 2-(4′-chloro-4-biphenylyl)-1-{[(3S)-1-(cyclopropylcarbonyl)-3-pyrrolidinyl]methyl}-1H-imidazole; 1-{[(3S)-1-(cyclopropylcarbonyl)-3-pyrrol idinyl]methyl}-2-(2′,4′-dichloro-4-biphenylyl)-1H-imidazole; 1-{[(3S)-1-(cyclopropylcarbonyl)-3-pyrrolidinyl]methyl}-2-(4′-fluoro-4-biphenylyl)-1H-imidazole; 3-[4-(1-{[(3S)-1-(cyclopropylcarbonyl)-3-pyrrolidinyl]methyl}-1H-imidazol-2-yl)phenyl]pyridine; 6-[4-(1-{[1-(cyclopropylcarbonyl)-3-pyrrolidinyl]methyl}-1H-imidazol-2-yl)phenyl]-1H-indole; 2-[4-(1-benzofuran-5-yl)phenyl]-1-{[(3S)-1-(cyclopropylcarbonyl)-3-pyrrolidinyl]methyl}-1H-imidazole; 1-{[1-(cyclopropylcarbonyl)-3-pyrrolidinyl]methyl}-4,5-dimethyl-2-[4′-(methyloxy)-4-biphenylyl]-1H-imidazole; 1-{[1-(cyclopropylcarbonyl)-3-pyrrolidinyl]methyl}-2-(2′,4′-dichloro-4-biphenylyl)-4,5-dimethyl-1H-imidazole; 2-[2′-chloro-4′-(methyloxy)-4-biphenylyl]-1-{[1-(cyclopropylcarbonyl)-3-pyrrolidinyl]methyl}-4,5-dimethyl-1H-imidazole; 2-(3′-chloro-4′-fluoro-4-biphenylyl)-1-{[1-(cyclopropylcarbonyl)-3-pyrrolidinyl]methyl}-4,5-dimethyl-1H-imidazole; 1-{[1-(cyclopropylcarbonyl)-3-pyrrolidinyl]methyl}-2-(4′-fluoro-3′-methyl-4-biphenylyl)-4,5-dimethyl-1H-imidazole; 1-{[1-(cyclopropylcarbonyl)-3-pyrrolidinyl]methyl}-4,5-dimethyl-2-(4′-methyl-4-biphenylyl)-1H-imidazole; 1-{[1-(cyclopropylcarbonyl)-3-pyrrolidinyl]methyl}-2-(4′-fluoro-4-biphenylyl)-4,5-dimethyl-1H-imidazole; 4′-(1-{[1-(cyclopropylcarbonyl)-3-pyrrolidinyl]methyl}-4,5-dimethyl-1H-imidazol-2-yl)-3-biphenylol; 1-{[1-(cyclopropylcarbonyl)-3-pyrrolidinyl]methyl}-4,5-dimethyl-2-(3′-methyl-4-biphenylyl)-1H-imidazole; 2-(3′-chloro-4-biphenylyl)-1-{[1-(cyclopropylcarbonyl)-3-pyrrolidinyl]methyl}-4,5-dimethyl-1H-imidazole; 2-(4′-chloro-4-biphenylyl)-1-{[1-(cyclopropylcarbonyl)-3-pyrrolidinyl]methyl}-4,5-dimethyl-1H-imidazole; 1-{[1-(cyclopropylcarbonyl)-3-pyrrolidinyl]methyl}-5-methyl-2-(4′-methyl-4-biphenylyl)-1H-imidazole; 1-{[1-(cyclopropylcarbonyl)-3-pyrrolidinyl]methyl}-2-[4′-(ethyloxy)-4-biphenylyl]-5-methyl-1H-imidazole; 1-{[1-(cyclopropylcarbonyl)-3-pyrrolidinyl]methyl}-5-methyl-2-[4′-(methyloxy)-4-biphenylyl]-1H-imidazole; 2-(4′-chloro-4-biphenylyl)-1-{[1-(cyclopropylcarbonyl)-3-pyrrolidinyl]methyl}-5-methyl-1H-imidazole; 1-{[1-(cyclopropylcarbonyl)-3-pyrrolidinyl]methyl}-5-methyl-2-(3′-methyl-4-biphenylyl)-1H-imidazole; 2-(3′-chloro-4-biphenylyl)-1-{[1-(cyclopropylcarbonyl)-3-pyrrolidinyl]methyl}-5-methyl-1H-imidazole; 1-{[1-(cyclopropylcarbonyl)-3-pyrrolidinyl]methyl}-2-(4′-fluoro-4-biphenylyl)-5-methyl-1H-imidazole; 1-{[1-(cyclopropylcarbonyl)-3-pyrrolidinyl]methyl}-2-(2′,4′-dimethyl-4-biphenylyl)-5-methyl-1H-imidazole; 1-{[1-(cyclopropylcarbonyl)-3-pyrrolidinyl]methyl}-2-(2′,4′-dichloro-4-biphenylyl)-5-methyl-1H-imidazole; 1-{[1-(cyclopropylcarbonyl)-3-pyrrolidinyl]methyl}-2-(4′-fluoro-3′-methyl-4-biphenylyl)-5-methyl-1H-imidazole; 2-[2′-chloro-4′-(methyloxy)-4-biphenylyl]-1-{[1-(cyclopropylcarbonyl)-3-pyrrolidinyl]methyl}-5-methyl-1H-imidazole; 2-(3′-chloro-4′-fluoro-4-biphenylyl)-1-{[1-(cyclopropylcarbonyl)-3-pyrrolidinyl]methyl}-5-methyl-1H-imidazole; 3-[4-(1-benzofuran-5-yl)phenyl]-4-{[(3S)-1-(cyclopropylcarbonyl)-3-pyrrolidinyl]methyl}-4H-1,2,4-triazole; 5-[4-(4-{[(3S)-1-(cyclopropylcarbonyl)-3-pyrrolidinyl]methyl}-4H-1,2,4-triazol-3-yl)phenyl]-1H-indole; 5-[4-(4-{[(3S)-1-(cyclopropylcarbonyl)-3-pyrrolidinyl]methyl}-4H-1,2,4-triazol-3-yl)phenyl]-1H-indazole; 4-{[(3S)-1-(cyclopropylcarbonyl)-3-pyrrolidinyl]methyl}-3-[4′-(methyloxy)-4-biphenylyl]-4H-1,2,4-triazole; 4′-(4-{[(3S)-1-(cyclopropylcarbonyl)-3-pyrrolidinyl]methyl}-4H-1,2,4-triazol-3-yl)-4-biphenylcarbonitrile; 4-{[(3S)-1-(cyclopropylcarbonyl)-3-pyrrolidinyl]methyl}-3-(2′,4′-difluoro-4-biphenylyl)-4H-1,2,4-triazole; 4′-(4-{[(3S)-1-(cyclopropylcarbonyl)-3-pyrrolidinyl]methyl}-4H-1,2,4-triazol-3-yl)-2-biphenylcarbonitrile; 6-[4-(4-{[(3S)-1-(cyclopropylcarbonyl)-3-pyrrolidinyl]methyl}-4H-1,2,4-triazol-3-yl)phenyl]-1H-pyrrolo[3,2-b]pyridine; 4-[4-(4-{[(3S)-1-(cyclopropylcarbonyl)-3-pyrrolidinyl]methyl}-4H-1,2,4-triazol-3-yl)phenyl]-1H-indole; 4-[4-(4-{[(3S)-1-(cyclopropylcarbonyl)-3-pyrrolidinyl]methyl}-4H-1,2,4-triazol-3-yl)phenyl]-1H-indazole; 7-[4-(4-{[(3S)-1-(cyclopropylcarbonyl)-3-pyrrolidinyl]methyl}-4H-1,2,4-triazol-3-yl)phenyl]imidazo[1,2-a]pyridine; N-[4′-(4-{[(3S)-1-(cyclopropylcarbonyl)-3-pyrrolidinyl]methyl}-4H-1,2,4-triazol-3-yl)-3-biphenylyl]-N,N-dimethylsulfamide; 6-[4-(4-{[(3S)-1-(cyclopropylcarbonyl)-3-pyrrolidinyl]methyl}-4H-1,2,4-triazol-3-yl)-3-fluorophenyl]-1H-indole; 3-[4-(1-benzofuran-5-yl)-2-fluorophenyl]-4-{[(3S)-1-(cyclopropylcarbonyl)-3-pyrrolidinyl]methyl}-4H-1,2,4-triazole; 5-[4-(4-{[(3S)-1-(cyclopropylcarbonyl)-3-pyrrolidinyl]methyl}-4H-1,2,4-triazol-3-yl)phenyl]-2,3-dihydro-1H-indole; 5-[4-(4-{[(3S)-1-(cyclopropylcarbonyl)-3-pyrrolidinyl]methyl}-4H-1,2,4-triazol-3-yl)phenyl]-1-methyl-1H-pyrrolo[2,3-b]pyridine; 4-{[(3S)-1-(cyclopropylcarbonyl)-3-pyrrolidinyl]methyl}-3-[4-(2,3-dihydro-1-benzofuran-5-yl)phenyl]-4H-1,2,4-triazole; 5-[4-(4-{[(3S)-1-(cyclopropylcarbonyl)-3-pyrrolidinyl]methyl}-4H-1,2,4-triazol-3-yl)phenyl]-1-methyl-1H-indole; 5-[4′-(4-{[(3S)-1-(cyclopropylcarbonyl)-3-pyrrolidinyl]methyl}-4H-1,2,4-triazol-3-yl)-3-biphenylyl]-1H-tetrazole; 6-[4-(4-{[(3S)-1-(cyclopropylcarbonyl)-3-pyrrolidinyl]methyl}-4H-1,2,4-triazol-3-yl)phenyl]-1H-indazole; 5-[4-(4-{[1-(cyclopropylcarbonyl)-3-azetidinyl]methyl}-4H-1,2,4-triazol-3-yl)phenyl]-1H-indazole; 6-[4-(4-{[1-(cyclopropylcarbonyl)-3-azetidinyl]methyl}-4H-1,2,4-triazol-3-yl)phenyl]-1H-indole; 6-[4-(4-{[1-(cyclopropylcarbonyl)-3-azetidinyl]methyl}-4H-1,2,4-triazol-3-yl)phenyl]-1,3-benzothiazole; 4-{[(3S)-1-(cyclopropylcarbonyl)-3-pyrrolidinyl]methyl}-3-(3′-methyl-4-biphenylyl)-4H-1,2,4-triazole; 4-{[(3S)-1-(cyclopropylcarbonyl)-3-pyrrolidinyl]methyl}-3-(4′-methyl-4-biphenylyl)-4H-1,2,4-triazole; 3-(3′-chloro-4-biphenylyl)-4-{[(3S)-1-(cyclopropylcarbonyl)-3-pyrrolidinyl]methyl}-4H-1,2,4-triazole; 3-(4′-chloro-4-biphenylyl)-4-{[(3S)-1-(cyclopropylcarbonyl)-3-pyrrolidinyl]methyl}-4H-1,2,4-triazole; 6-[4-(4-{[1-(cyclopropylcarbonyl)-3-azetidinyl]methyl}-4H-1,2,4-triazol-3-yl)-3-fluorophenyl]-1H-indole; 4-{[(3S)-1-(cyclopropylcarbonyl)-3-pyrrolidinyl]methyl}-3-[3-fluoro-4′-(1H-pyrazol-1-yl)-4-biphenylyl]-4H-1,2,4-triazole; 4-{[(3S)-1-(cyclopropylcarbonyl)-3-pyrrolidinyl]methyl}-3-[3-fluoro-3′-(1H-pyrazol-5-yl)-4-biphenylyl]-4H-1,2,4-triazole; 4′-(4-{[(3S)-1-(cyclopropylcarbonyl)-3-pyrrolidinyl]methyl}-4H-1,2,4-triazol-3-yl)-3-biphenylol; 4′-(4-{[(3S)-1-(cyclopropylcarbonyl)-3-pyrrolidinyl]methyl}-4H-1,2,4-triazol-3-yl)-4-biphenylol; 4-{[(3S)-1-(cyclopropylcarbonyl)-3-pyrrolidinyl]methyl}-3-(2′,3,4′-trifluoro-4-biphenylyl)-4H-1,2,4-triazole; 4-{[(3S)-1-(cyclopropylcarbonyl)-3-pyrrolidinyl]methyl}-3-(3′,3,4′-trifluoro-4-biphenylyl)-4H-1,2,4-triazole; 4-{[(3S)-1-(cyclopropylcarbonyl)-3-pyrrolidinyl]methyl}-3-[3,4′-difluoro-3′-(methyl)-4-biphenylyl]-4H-1,2,4-triazole; 4-{[(3S)-1-(cyclopropylcarbonyl)-3-pyrrolidinyl]methyl}-3-[3,4′-difluoro-3′-(methyloxy)-4-biphenylyl]-4H-1,2,4-triazole; 4-{[(3S)-1-(cyclopropylcarbonyl)-3-pyrrolidinyl]methyl}-3-[2′,3-difluoro-4′-(methyloxy)-4-biphenylyl]-4H-1,2,4-triazole; 4-{[(3S)-1-(cyclopropylcarbonyl)-3-pyrrolidinyl]methyl}-3-[3′,3-difluoro-4′-(methyloxy)-4-biphenylyl]-4H-1,2,4-triazole; 6-[4-(4-{[1-(cyclopropylcarbonyl)-3-azetidinyl]methyl}-4H-1,2,4-triazol-3-yl)-3-fluorophenyl]quinoline; 4-{[(3S)-1-(cyclopropylcarbonyl)-3-pyrrolidinyl]methyl}-3-(2′,3-difluoro-4′-methyl-4-biphenylyl)-4H-1,2,4-triazole; 4-{[(3S)-1-(cyclopropylcarbonyl)-3-pyrrolidinyl]methyl}-3-[3-fluoro-4′-methyl-3′-(methyloxy)-4-biphenylyl]-4H-1,2,4-triazole; 4-{[(3S)-1-(cyclopropylcarbonyl)-3-pyrrolidinyl]methyl}-3-[3-fluoro-3′-methyl-4′-(methyloxy)-4-biphenylyl]-4H-1,2,4-triazole; 3-[3′-chloro-3-fluoro-4′-(methyloxy)-4-biphenylyl]-4-{[(3S)-1-(cyclopropylcarbonyl)-3-pyrrolidinyl]methyl}-4H-1,2,4-triazole; 7-[4-(4-{[1-(cyclopropylcarbonyl)-3-azetidinyl]methyl}-4H-1,2,4-triazol-3-yl)-3-fluorophenyl]quinoline; 4′-(4-{[(3S)-1-(cyclopropylcarbonyl)-3-pyrrolidinyl]methyl}-4H-1,2,4-triazol-3-yl)-3′,4-difluoro-3-biphenylol; 4′-(4-{[(3S)-1-(cyclopropylcarbonyl)-3-pyrrolidinyl]methyl}-4H-1,2,4-triazol-3-yl)-3′-fluoro-4-(methyloxy)-3-biphenylol; 4′-(4-{[(3S)-1-(cyclopropylcarbonyl)-3-pyrrolidinyl]methyl}-4H-1,2,4-triazol-3-yl)-3′-fluoro-4-biphenylcarbonitrile; 4-{[(3S)-1-(cyclopropylcarbonyl)-3-pyrrolidinyl]methyl}-3-(3,4′-difluoro-4-biphenylyl)-4H-1,2,4-triazole; 4′-(4-{[(3S)-1-(cyclopropylcarbonyl)-3-pyrrolidinyl]methyl}-4H-1,2,4-triazol-3-yl)-3′-fluoro-N,N-dimethyl-4-biphenylamine; 7-[4-(4-{[(3S)-1-(cyclopropylcarbonyl)-3-pyrrolidinyl]methyl}-4H-1,2,4-triazol-3-yl)-3-fluorophenyl]quinoline; 3-[4-(4-{[(3S)-1-(cyclopropylcarbonyl)-3-pyrrolidinyl]methyl}-4H-1,2,4-triazol-3-yl)-3-fluorophenyl]quinoline; or 4′-(4-{[(3S)-1-(cyclopropylcarbonyl)-3-pyrrolidinyl]methyl}-4H-1,2,4-triazol-3-yl)-3′-fluoro-4-methyl-3-biphenylol; or pharmaceutically acceptable salts thereof.

In some embodiments, the compound is one of the following:

| | Compound |
|---|---|
| 1182 | 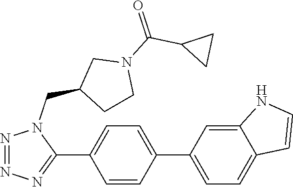 [SMILES: O=C(C1CC1)N1CC[C@@H](Cn2nnnc2-c2ccc(-c3ccc4cc[nH]c4c3)cc2)C1] |
| 1183 | 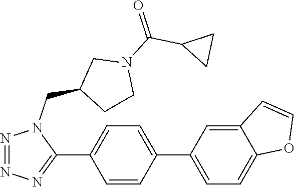 [SMILES: O=C(C1CC1)N1CC[C@@H](Cn2nnnc2-c2ccc(-c3ccc4occc4c3)cc2)C1] |
| 1184 | 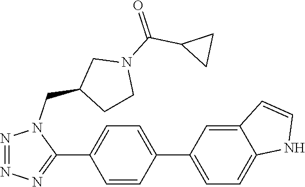 [SMILES: O=C(C1CC1)N1CC[C@@H](Cn2nnnc2-c2ccc(-c3ccc4[nH]ccc4c3)cc2)C1] |
| 1185 | 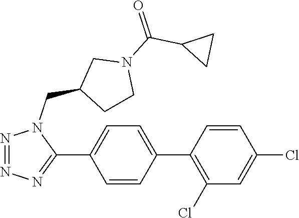 [SMILES: O=C(C1CC1)N1CC[C@@H](Cn2nnnc2-c2ccc(-c3ccc(Cl)cc3Cl)cc2)C1] |
| 1186 | 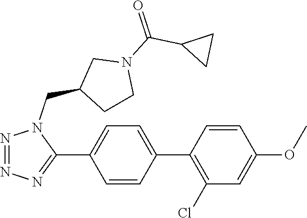 [SMILES: COc1ccc(-c2ccc(-c3nnnn3C[C@@H]3CCN(C(=O)C4CC4)C3)cc2)c(Cl)c1] |
| 1187 | 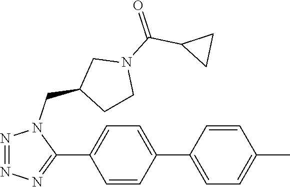 [SMILES: Cc1ccc(-c2ccc(-c3nnnn3C[C@@H]3CCN(C(=O)C4CC4)C3)cc2)cc1] |
| 1188 | 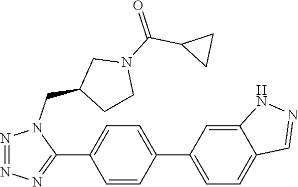 [SMILES: O=C(C1CC1)N1CC[C@@H](Cn2nnnc2-c2ccc(-c3ccc4cn[nH]c4c3)cc2)C1] |
| 1189 | 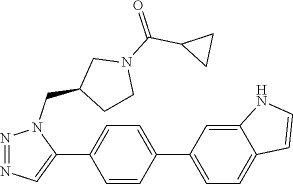 [SMILES: O=C(C1CC1)N1CC[C@@H](Cn2nncc2-c2ccc(-c3ccc4cc[nH]c4c3)cc2)C1] |
| 1190 | 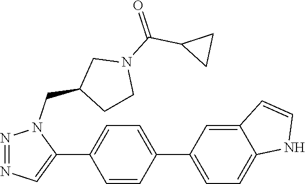 [SMILES: O=C(C1CC1)N1CC[C@@H](Cn2nncc2-c2ccc(-c3ccc4[nH]ccc4c3)cc2)C1] |
| 1191 | 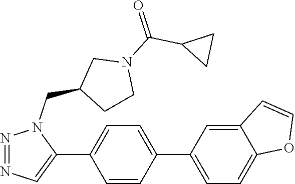 [SMILES: O=C(C1CC1)N1CC[C@@H](Cn2nncc2-c2ccc(-c3ccc4occc4c3)cc2)C1] |
| 1192 | 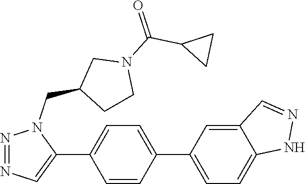 [SMILES: O=C(C1CC1)N1CC[C@@H](Cn2nncc2-c2ccc(-c3ccc4[nH]ncc4c3)cc2)C1] |
| 1193 | 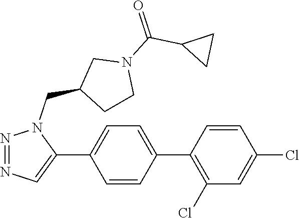 [SMILES: O=C(C1CC1)N1CC[C@@H](Cn2nncc2-c2ccc(-c3ccc(Cl)cc3Cl)cc2)C1] |
| 1194 | 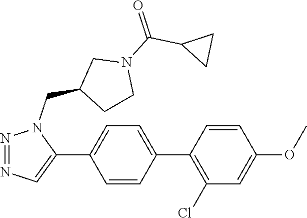 [SMILES: COc1ccc(-c2ccc(-c3cnnn3C[C@@H]3CCN(C(=O)C4CC4)C3)cc2)c(Cl)c1] |
| 1195 | 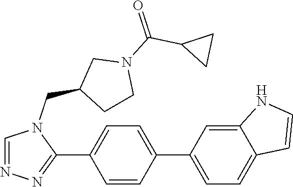 [SMILES: O=C(C1CC1)N1CC[C@@H](Cn2cnnc2-c2ccc(-c3ccc4cc[nH]c4c3)cc2)C1] |
| 1196 | 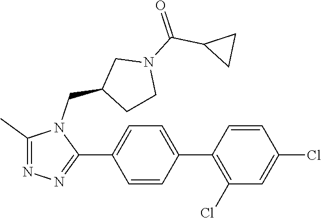 [SMILES: Cc1nnc(-c2ccc(-c3ccc(Cl)cc3Cl)cc2)n1C[C@@H]1CCN(C(=O)C2CC2)C1] |
| 1197 | 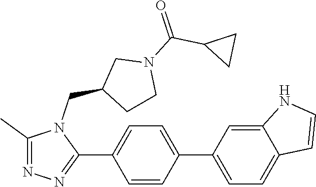 [SMILES: Cc1nnc(-c2ccc(-c3ccc4cc[nH]c4c3)cc2)n1C[C@@H]1CCN(C(=O)C2CC2)C1] |
| 1198 | 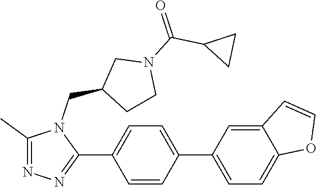 [SMILES: Cc1nnc(-c2ccc(-c3ccc4occc4c3)cc2)n1C[C@@H]1CCN(C(=O)C2CC2)C1] |
| 1199 | 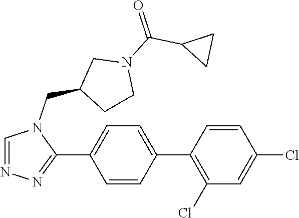 [SMILES: O=C(C1CC1)N1CC[C@@H](Cn2cnnc2-c2ccc(-c3ccc(Cl)cc3Cl)cc2)C1] |
| 1200 | 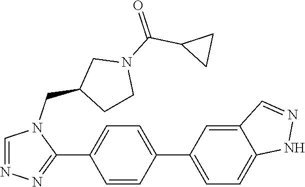 [SMILES: O=C(C1CC1)N1CC[C@@H](Cn2cnnc2-c2ccc(-c3ccc4[nH]ncc4c3)cc2)C1] |
| 1201 | 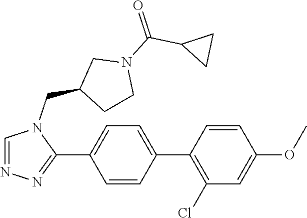 [SMILES: COc1ccc(-c2ccc(-c3nncn3C[C@@H]3CCN(C(=O)C4CC4)C3)cc2)c(Cl)c1] |
| 1202 | 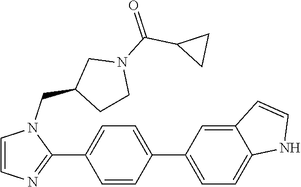 [SMILES: O=C(C1CC1)N1CC[C@@H](Cn2ccnc2-c2ccc(-c3ccc4[nH]ccc4c3)cc2)C1] |
| 1203 | 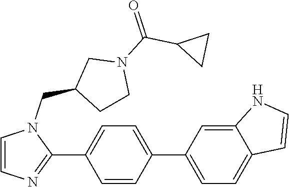 [SMILES: O=C(C1CC1)N1CC[C@@H](Cn2ccnc2-c2ccc(-c3ccc4cc[nH]c4c3)cc2)C1] |
| 1204 | 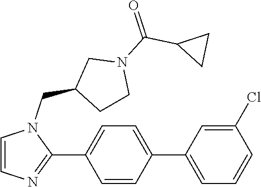 [SMILES: O=C(C1CC1)N1CC[C@@H](Cn2ccnc2-c2ccc(-c3cccc(Cl)c3)cc2)C1] |
| 1205 | 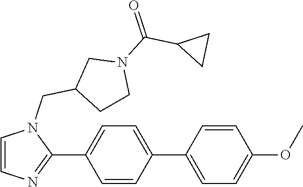 [SMILES: COc1ccc(-c2ccc(-c3nccn3CC3CCN(C(=O)C4CC4)C3)cc2)cc1] |
| 1206 | 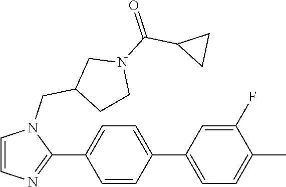 [SMILES: Cc1ccc(-c2ccc(-c3nccn3CC3CCN(C(=O)C4CC4)C3)cc2)cc1F] |
| 1207 | 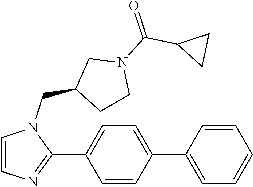 [SMILES: O=C(C1CC1)N1CC[C@@H](Cn2ccnc2-c2ccc(-c3ccccc3)cc2)C1] |
| 1208 | 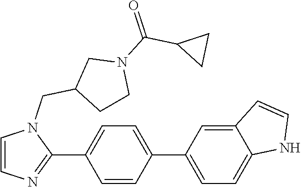 [SMILES: O=C(C1CC1)N1CCC(Cn2ccnc2-c2ccc(-c3ccc4[nH]ccc4c3)cc2)C1] |
| 1209 | 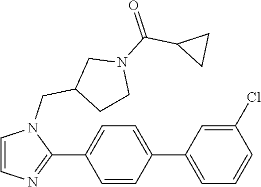 [SMILES: O=C(C1CC1)N1CCC(Cn2ccnc2-c2ccc(-c3cccc(Cl)c3)cc2)C1] |
| 1210 | 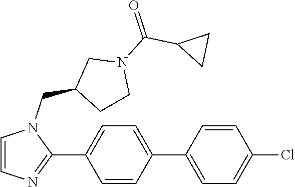 [SMILES: O=C(C1CC1)N1CC[C@@H](Cn2ccnc2-c2ccc(-c3ccc(Cl)cc3)cc2)C1] |
| 1211 | 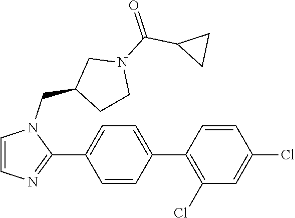 [SMILES: O=C(C1CC1)N1CC[C@@H](Cn2ccnc2-c2ccc(-c3ccc(Cl)cc3Cl)cc2)C1] |
| 1212 | 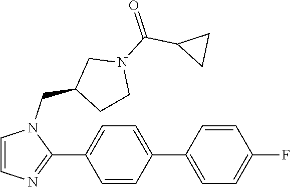 [SMILES: O=C(C1CC1)N1CC[C@@H](Cn2ccnc2-c2ccc(-c3ccc(F)cc3)cc2)C1] |
| 1213 | 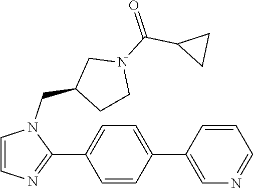 [SMILES: O=C(C1CC1)N1CC[C@@H](Cn2ccnc2-c2ccc(-c3cccnc3)cc2)C1] |
| 1214 | 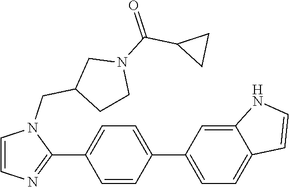 [SMILES: O=C(C1CC1)N1CCC(Cn2ccnc2-c2ccc(-c3ccc4cc[nH]c4c3)cc2)C1] |
| 1215 | 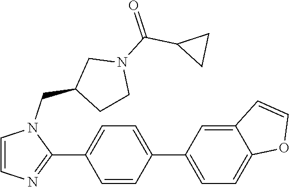 [SMILES: O=C(C1CC1)N1CC[C@@H](Cn2ccnc2-c2ccc(-c3ccc4occc4c3)cc2)C1] |
| 1216 | 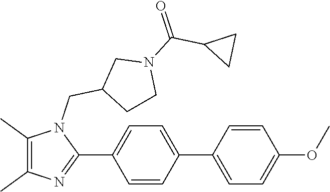 |
| 1217 | 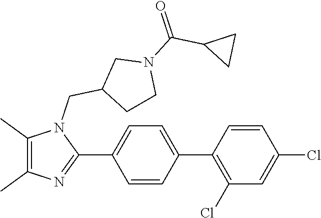 [SMILES: Cc1nc(-c2ccc(-c3ccc(Cl)cc3Cl)cc2)n(CC2CCN(C(=O)C3CC3)C2)c1C] |
| 1218 | 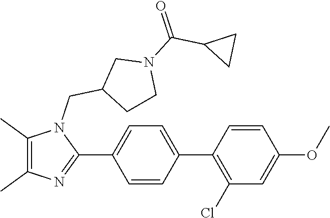 |
| 1219 | 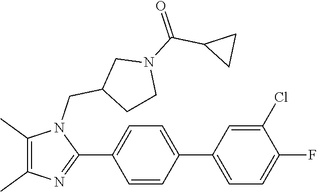 [SMILES: Cc1nc(-c2ccc(-c3ccc(F)c(Cl)c3)cc2)n(CC2CCN(C(=O)C3CC3)C2)c1C] |
| 1220 | 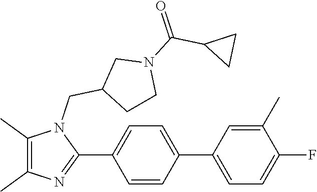 [SMILES: Cc1cc(-c2ccc(-c3nc(C)c(C)n3CC3CCN(C(=O)C4CC4)C3)cc2)ccc1F] |
| 1221 | 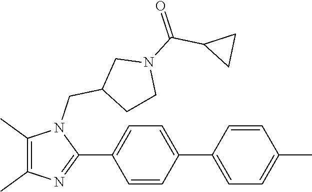 |
| 1222 | 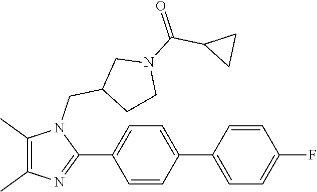 [SMILES: Cc1nc(-c2ccc(-c3ccc(F)cc3)cc2)n(CC2CCN(C(=O)C3CC3)C2)c1C] |
| 1223 | 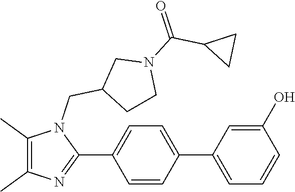 [SMILES: Cc1nc(-c2ccc(-c3cccc(O)c3)cc2)n(CC2CCN(C(=O)C3CC3)C2)c1C] |
| 1224 | 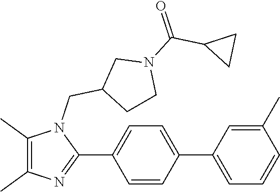 |
| 1225 | 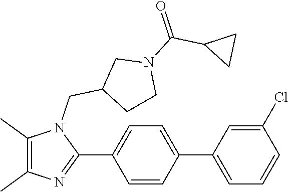 [SMILES: Cc1nc(-c2ccc(-c3cccc(Cl)c3)cc2)n(CC2CCN(C(=O)C3CC3)C2)c1C] |
| 1226 | 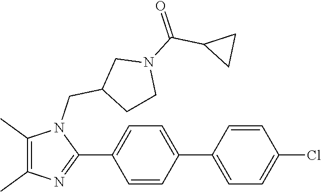 [SMILES: Cc1nc(-c2ccc(-c3ccc(Cl)cc3)cc2)n(CC2CCN(C(=O)C3CC3)C2)c1C] |
| 1227 | 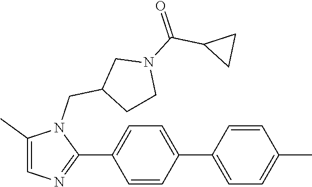 |
| 1228 | 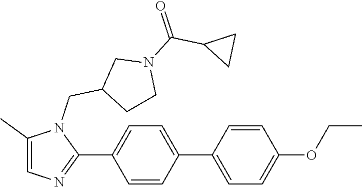 [SMILES: CCOc1ccc(-c2ccc(-c3ncc(C)n3CC3CCN(C(=O)C4CC4)C3)cc2)cc1] |
| 1229 | 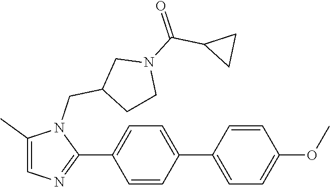 |
| 1230 | 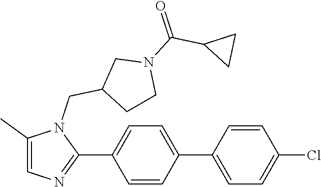 [SMILES: Cc1cnc(-c2ccc(-c3ccc(Cl)cc3)cc2)n1CC1CCN(C(=O)C2CC2)C1] |
| 1231 | 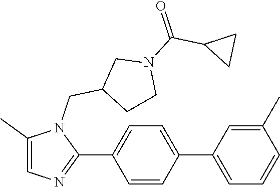 |
| 1232 | 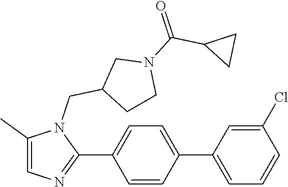 [SMILES: Cc1cnc(-c2ccc(-c3cccc(Cl)c3)cc2)n1CC1CCN(C(=O)C2CC2)C1] |
| 1233 | 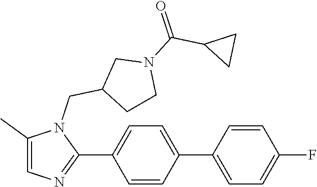 [SMILES: Cc1cnc(-c2ccc(-c3ccc(F)cc3)cc2)n1CC1CCN(C(=O)C2CC2)C1] |
| 1234 | 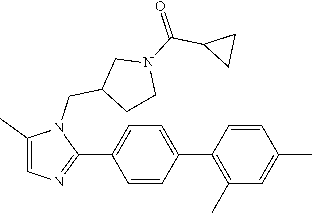 |
| 1235 | 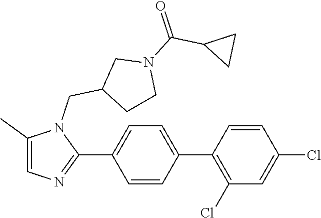 [SMILES: Cc1cnc(-c2ccc(-c3ccc(Cl)cc3Cl)cc2)n1CC1CCN(C(=O)C2CC2)C1] |
| 1236 | 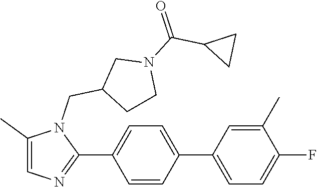 [SMILES: Cc1cc(-c2ccc(-c3ncc(C)n3CC3CCN(C(=O)C4CC4)C3)cc2)ccc1F] |
| 1237 | 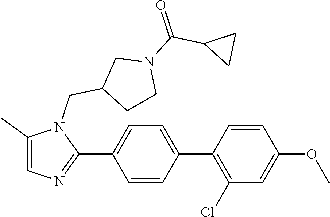 |
| 1238 | 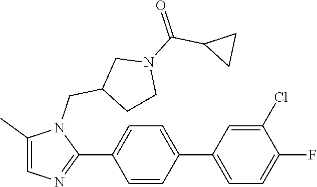 [SMILES: Cc1cnc(-c2ccc(-c3ccc(F)c(Cl)c3)cc2)n1CC1CCN(C(=O)C2CC2)C1] |
| 1239 | 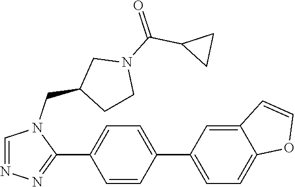 [SMILES: O=C(C1CC1)N1CC[C@@H](Cn2cnnc2-c2ccc(-c3ccc4occc4c3)cc2)C1] |
| 1240 | 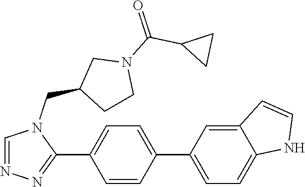 [SMILES: O=C(C1CC1)N1CC[C@@H](Cn2cnnc2-c2ccc(-c3ccc4[nH]ccc4c3)cc2)C1] |
| 1241 | 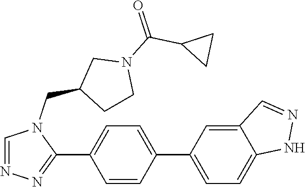 [SMILES: O=C(C1CC1)N1CC[C@@H](Cn2cnnc2-c2ccc(-c3ccc4[nH]ncc4c3)cc2)C1] |
| 1242 | 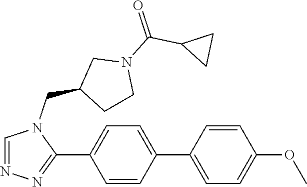 [SMILES: COc1ccc(-c2ccc(-c3nncn3C[C@@H]3CCN(C(=O)C4CC4)C3)cc2)cc1] |
| 1243 | 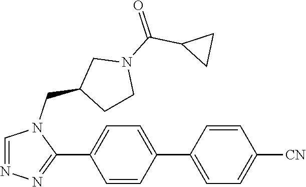 [SMILES: N#Cc1ccc(-c2ccc(-c3nncn3C[C@@H]3CCN(C(=O)C4CC4)C3)cc2)cc1] |
| 1244 | 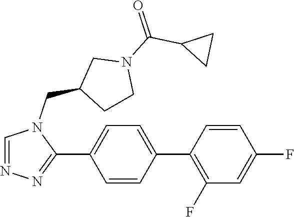 [SMILES: O=C(C1CC1)N1CC[C@@H](Cn2cnnc2-c2ccc(-c3ccc(F)cc3F)cc2)C1] |
| 1245 | 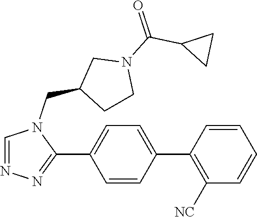 [SMILES: N#Cc1ccccc1-c1ccc(-c2nncn2C[C@@H]2CCN(C(=O)C3CC3)C2)cc1] |
| 1246 | 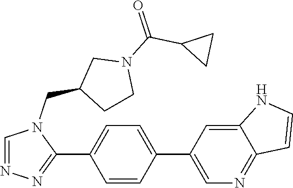 [SMILES: O=C(C1CC1)N1CC[C@@H](Cn2cnnc2-c2ccc(-c3cnc4cc[nH]c4c3)cc2)C1] |
| 1247 | 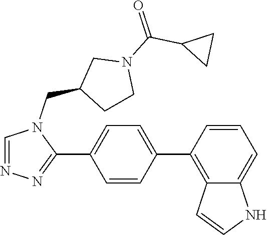 [SMILES: O=C(C1CC1)N1CC[C@@H](Cn2cnnc2-c2ccc(-c3cccc4[nH]ccc34)cc2)C1] |
| 1248 | 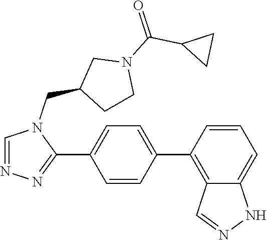 [SMILES: O=C(C1CC1)N1CC[C@@H](Cn2cnnc2-c2ccc(-c3cccc4[nH]ncc34)cc2)C1] |
| 1249 | 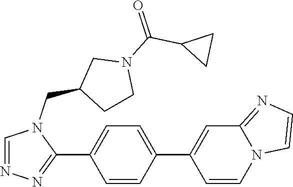 [SMILES: O=C(C1CC1)N1CC[C@@H](Cn2cnnc2-c2ccc(-c3ccn4ccnc4c3)cc2)C1] |
| 1250 | 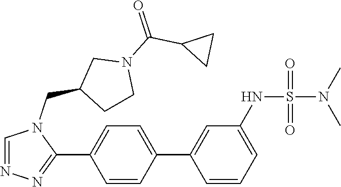 [SMILES: CN(C)S(=O)(=O)Nc1cccc(-c2ccc(-c3nncn3C[C@@H]3CCN(C(=O)C4CC4)C3)cc2)c1] |
| 1251 | 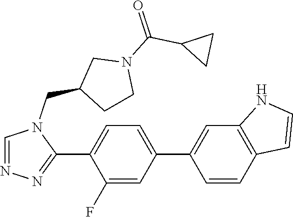 [SMILES: O=C(C1CC1)N1CC[C@@H](Cn2cnnc2-c2ccc(-c3ccc4cc[nH]c4c3)cc2F)C1] |
| 1252 | 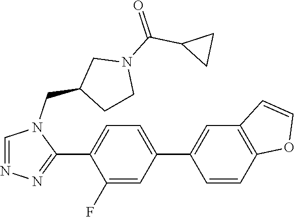 [SMILES: O=C(C1CC1)N1CC[C@@H](Cn2cnnc2-c2ccc(-c3ccc4occc4c3)cc2F)C1] |
| 1253 | 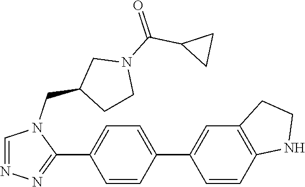 [SMILES: O=C(C1CC1)N1CC[C@@H](Cn2cnnc2-c2ccc(-c3ccc4c(c3)CCN4)cc2)C1] |
| 1254 | 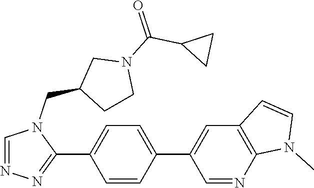 [SMILES: Cn1ccc2cc(-c3ccc(-c4nncn4C[C@@H]4CCN(C(=O)C5CC5)C4)cc3)cnc21] |
| 1255 | 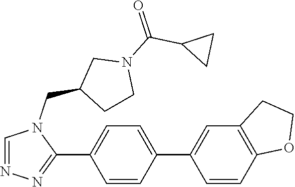 [SMILES: O=C(C1CC1)N1CC[C@@H](Cn2cnnc2-c2ccc(-c3ccc4c(c3)CCO4)cc2)C1] |
| 1256 | 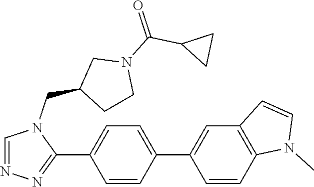 [SMILES: Cn1ccc2cc(-c3ccc(-c4nncn4C[C@@H]4CCN(C(=O)C5CC5)C4)cc3)ccc21] |
| 1257 | 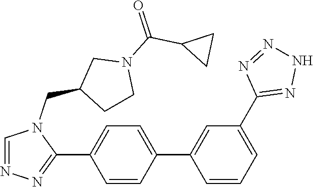 [SMILES: O=C(C1CC1)N1CC[C@@H](Cn2cnnc2-c2ccc(-c3cccc(-c4nn[nH]n4)c3)cc2)C1] |
| 1258 | 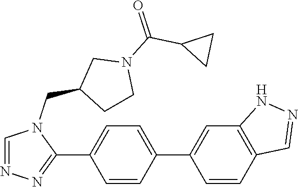 [SMILES: O=C(C1CC1)N1CC[C@@H](Cn2cnnc2-c2ccc(-c3ccc4cn[nH]c4c3)cc2)C1] |
| 1259 | 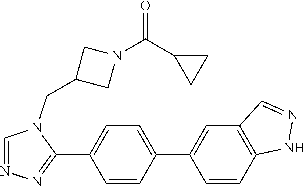 [SMILES: O=C(C1CC1)N1CC(Cn2cnnc2-c2ccc(-c3ccc4[nH]ncc4c3)cc2)C1] |
| 1260 | 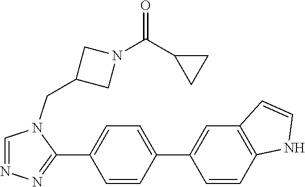 [SMILES: O=C(C1CC1)N1CC(Cn2cnnc2-c2ccc(-c3ccc4[nH]ccc4c3)cc2)C1] |
| 1261 | 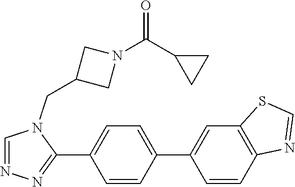 [SMILES: O=C(C1CC1)N1CC(Cn2cnnc2-c2ccc(-c3ccc4ncsc4c3)cc2)C1] |
| 1262 | 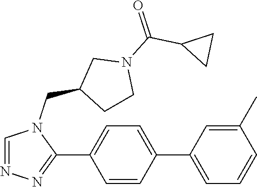 [SMILES: Cc1cccc(-c2ccc(-c3nncn3C[C@@H]3CCN(C(=O)C4CC4)C3)cc2)c1] |
| 1263 | 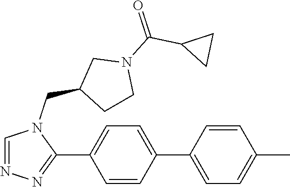 [SMILES: Cc1ccc(-c2ccc(-c3nncn3C[C@@H]3CCN(C(=O)C4CC4)C3)cc2)cc1] |
| 1264 | 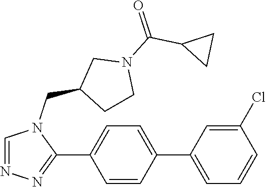 [SMILES: O=C(C1CC1)N1CC[C@@H](Cn2cnnc2-c2ccc(-c3cccc(Cl)c3)cc2)C1] |
| 1265 | 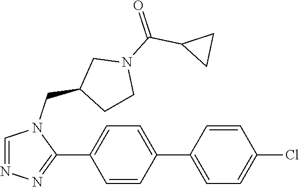 [SMILES: O=C(C1CC1)N1CC[C@@H](Cn2cnnc2-c2ccc(-c3ccc(Cl)cc3)cc2)C1] |
| 1266 | 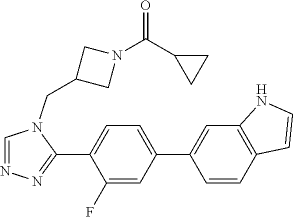 [SMILES: O=C(C1CC1)N1CC(Cn2cnnc2-c2ccc(-c3ccc4cc[nH]c4c3)cc2F)C1] |
| 1267 | 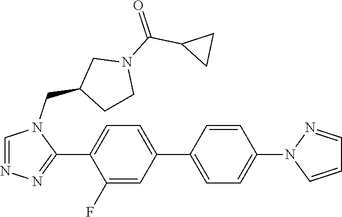 [SMILES: O=C(C1CC1)N1CC[C@@H](Cn2cnnc2-c2ccc(-c3ccc(-n4cccn4)cc3)cc2F)C1] |
| 1268 | 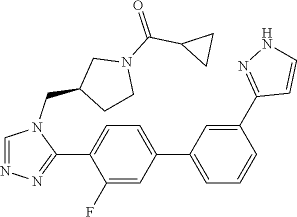 [SMILES: O=C(C1CC1)N1CC[C@@H](Cn2cnnc2-c2ccc(-c3cccc(-c4cc[nH]n4)c3)cc2F)C1] |
| 1269 | 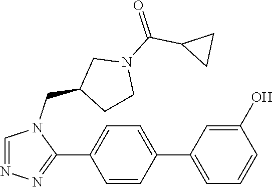 [SMILES: O=C(C1CC1)N1CC[C@@H](Cn2cnnc2-c2ccc(-c3cccc(O)c3)cc2)C1] |
| 1270 | 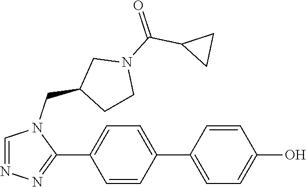 [SMILES: O=C(C1CC1)N1CC[C@@H](Cn2cnnc2-c2ccc(-c3ccc(O)cc3)cc2)C1] |
| 1271 | 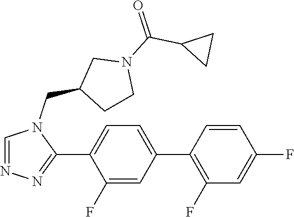 [SMILES: O=C(C1CC1)N1CC[C@@H](Cn2cnnc2-c2ccc(-c3ccc(F)cc3F)cc2F)C1] |
| 1272 | 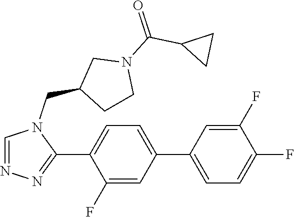 [SMILES: O=C(C1CC1)N1CC[C@@H](Cn2cnnc2-c2ccc(-c3ccc(F)c(F)c3)cc2F)C1] |
| 1273 | 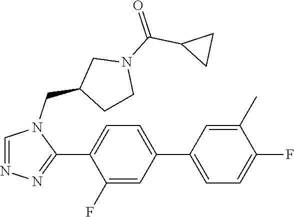 [SMILES: Cc1cc(-c2ccc(-c3nncn3C[C@@H]3CCN(C(=O)C4CC4)C3)c(F)c2)ccc1F] |
| 1274 | 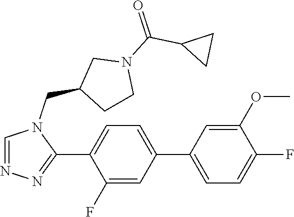 [SMILES: COc1cc(-c2ccc(-c3nncn3C[C@@H]3CCN(C(=O)C4CC4)C3)c(F)c2)ccc1F] |
| 1275 | 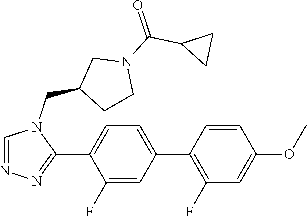 [SMILES: COc1ccc(-c2ccc(-c3nncn3C[C@@H]3CCN(C(=O)C4CC4)C3)c(F)c2)c(F)c1] |
| 1276 | 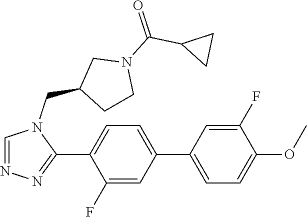 [SMILES: COc1ccc(-c2ccc(-c3nncn3C[C@@H]3CCN(C(=O)C4CC4)C3)c(F)c2)cc1F] |
| 1277 | 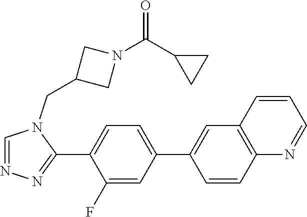 [SMILES: O=C(C1CC1)N1CC(Cn2cnnc2-c2ccc(-c3ccc4ncccc4c3)cc2F)C1] |
| 1278 | 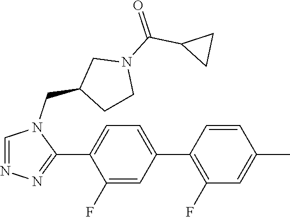 [SMILES: Cc1ccc(-c2ccc(-c3nncn3C[C@@H]3CCN(C(=O)C4CC4)C3)c(F)c2)c(F)c1] |
| 1279 | 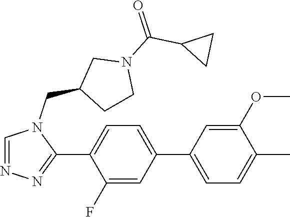 [SMILES: COc1cc(-c2ccc(-c3nncn3C[C@@H]3CCN(C(=O)C4CC4)C3)c(F)c2)ccc1C] |
| 1280 | 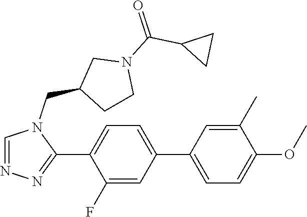 [SMILES: COc1ccc(-c2ccc(-c3nncn3C[C@@H]3CCN(C(=O)C4CC4)C3)c(F)c2)cc1C] |
| 1281 | 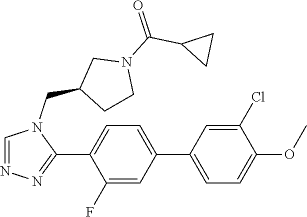 [SMILES: COc1ccc(-c2ccc(-c3nncn3C[C@@H]3CCN(C(=O)C4CC4)C3)c(F)c2)cc1Cl] |
| 1282 | 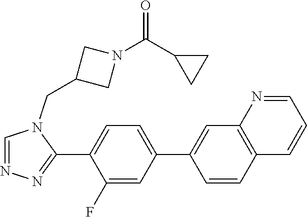 [SMILES: O=C(C1CC1)N1CC(Cn2cnnc2-c2ccc(-c3ccc4cccnc4c3)cc2F)C1] |
| 1283 | 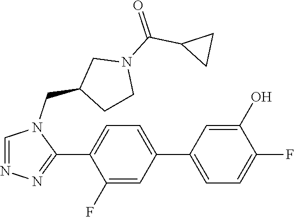 [SMILES: O=C(C1CC1)N1CC[C@@H](Cn2cnnc2-c2ccc(-c3ccc(F)c(O)c3)cc2F)C1] |
| 1284 | 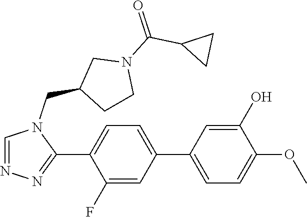 [SMILES: COc1ccc(-c2ccc(-c3nncn3C[C@@H]3CCN(C(=O)C4CC4)C3)c(F)c2)cc1O] |
| 1285 | 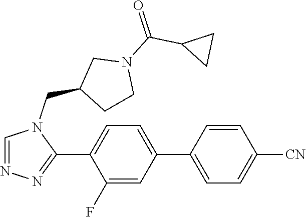 [SMILES: N#Cc1ccc(-c2ccc(-c3nncn3C[C@@H]3CCN(C(=O)C4CC4)C3)c(F)c2)cc1] |
| 1286 | 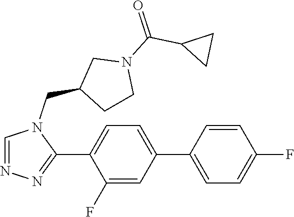 [SMILES: O=C(C1CC1)N1CC[C@@H](Cn2cnnc2-c2ccc(-c3ccc(F)cc3)cc2F)C1] |
| 1287 | 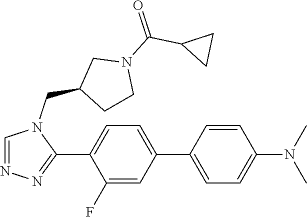 [SMILES: CN(C)c1ccc(-c2ccc(-c3nncn3C[C@@H]3CCN(C(=O)C4CC4)C3)c(F)c2)cc1] |
| 1288 | 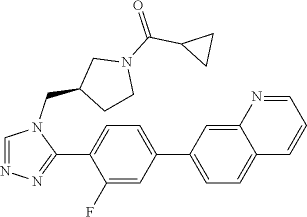 [SMILES: O=C(C1CC1)N1CC[C@@H](Cn2cnnc2-c2ccc(-c3ccc4cccnc4c3)cc2F)C1] |
| 1289 | 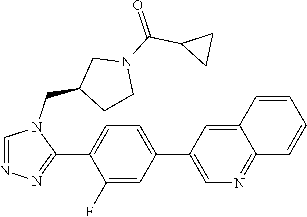 [SMILES: O=C(C1CC1)N1CC[C@@H](Cn2cnnc2-c2ccc(-c3cnc4ccccc4c3)cc2F)C1] |
| 1290 | 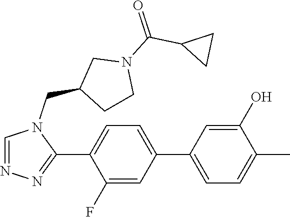 [SMILES: Cc1ccc(-c2ccc(-c3nncn3C[C@@H]3CCN(C(=O)C4CC4)C3)c(F)c2)cc1O] |

In some embodiments, the compound has the structure of Formula (XXV):

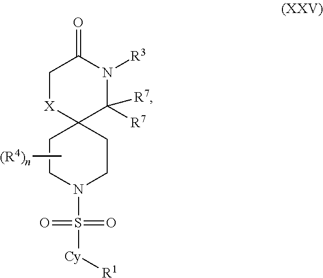

wherein R³ is selected from the group consisting of: C₁-C₆ alkyl, C₃-C₇ cycycloalkyl, and C₄-C₆ heterocycloalkyl, wherein said C₁-C₆ alkyl, C₃-C₇ cycloalkyl, or C₄-C₆ heterocycloalkyl is optionally substituted with from 1 to 6 substituents independently selected from the group of: halogen, C₁-C₄ alkyl, C₁-C₄ alkylhalogen, —CF₃, C₃-C₇ cycloalkyl, —C(O)C₁-C₄ alkyl, —C(O)C₃-C₇ cycloalkyl, —CO(phenyl), C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄ alkoxy, C₃-C₇ cycloalkoxy, hydroxy C₁-C₄ alkyl-, C₁-C₄ alkoxy C₁-C₄ alkyl-, —OCF₃, —NR⁵R⁶, R⁵R⁶NC₁-C₄ alkyl-, —NHC(O)C₁-C₄ alkyl, —NHCONR⁵R⁶, —NHSO₂C₁-C₄ alkyl, —NHSO₂NR⁵R⁶, and R⁹; R⁵ is selected from the group consisting of: hydrogen, C₁-C₄ alkyl, C₃-C₇cycloalkyl, —C₁-C₃ alkyl C₃-C₇ cycloalkyl, phenyl, and —C₁-C₃ alkylphenyl; R⁶ is hydrogen, C₁-C₄ alkyl, C₃-C₇ cycloalkyl, or —C₁-C₃ alkyl C₃-C₇ cycloalkyl; or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or C₁-C₄alkyl; R⁹ is a 5- or 6-membered heteroaryl ring containing 1 to 4 heteroatoms selected from oxygen, nitrogen, and sulfur, which is optionally substituted with 1 or 2 substituents selected from halogen, C₁-C₄ alkyl, CF₃, C₁-C₄ alkoxy, and —NR⁵R⁶; R⁴ is oxo, halogen or C₁-C₆ alkyl; Cy is selected from the group consisting of: phenyl, pyridinyl, and 5- or 6-membered heteroaryl wherein said phenyl, pyridinyl, and 5- or 6-membered heteroaryl are each optionally substituted with from one to three R² groups, wherein each R² is independently selected from C₁-C₆alkyl, cyano, C₁-C₄alkoxy, hydroxyl, —CF₃, or halogen; R¹ is selected from the group consisting of: phenyl, 5- or 6-membered heteroaryl, napthyl, and 9- or 10-membered heterocyclyl, wherein said phenyl, 5- or 6-membered heteroaryl, napthyl, or 9- or 10-membered heterocyclyl, is optionally substituted with from 1 to 4 substituents independently selected from halogen, C₁-C₄alkylhalogen, optionally substituted C₁-C₄ alkyl, —CF₃, —C₃-C₇ cycloalkyl, —C(O)C₁-C₄alkyl, —C(O)C₃-C₇ cycloalkyl, —CO(phenyl), —C₁-C₄(═O)OH, —C(═O)OC₁-C₄ alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxy C₁-C₄ alkyl-, C₁-C₄ alkoxy C₁-C₄ alkyl-, —OCF₃, —NR⁵R⁶, R⁵R⁶NC₁-C₄alkyl-, —NR⁶C(O)C₁-C₄alkyl, —NR⁶C(O) C₃-C₇ cycloalkyl, —NR⁶CONR⁵R⁶, —NR⁶SO₂C₁-C₄alkyl, —NR⁶SO₂NR⁵R⁶, —NR⁶C(O)H, tetrazolyl, —B(OH)₂, —SO₃H, and R⁹; each R⁷ is independently H, C₁-C₃alkyl, C₁-C₄ alkylhalogen, halogen, cyano, —CONR⁵R⁶, —C(═O)OC₁-C₄ alkyl, hydroxy C₁-C₄ alkyl-, and —C(═O)OH; X is CH₂, NR⁶ or O; n is 0, 1, 2, 3, or 4; or a pharmaceutically acceptable salt thereof.

In some embodiments, the compound has the structure of Formula (XXV-A):

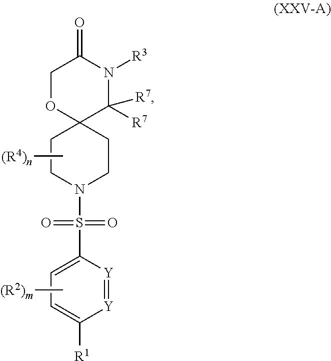

wherein R³ is selected from the group consisting of: C₁-C₆ alkyl, C₃-C₇ cycloalkyl, and C₄-C₆ heterocycloalkyl, wherein said C₁-C₆alkyl, C₃-C₇cycloalkyl or C₄-C₆ heterocycloalkyl is optionally substituted with from 1 to 6 substituents independently selected from the group of: halogen, C₁-C₄ alkyl, C₁-C₄ alkylhalogen, —CF₃, C₃-C₇ cycloalkyl, —C(O)C₁-C₄alkyl, —C(O)C₃-C₇ cycloalkyl, —CO(phenyl), —C₁-C₄(═O)OH, —C(═O)OC₁-C₄ alkyl, —CONR⁵R⁶, phenyl, —SO₂C₁-C₄ alkyl, —SO₂NR⁵R⁶, cyano, oxo, hydroxyl, C₁-C₄ alkoxy, C₃-C₇ cycloalkoxy, hydroxy C₁-C₄ alkyl-, C₁-C₄ alkoxy C₁-C₄ alkyl-, —OCF₃, —NR⁵R⁶, R⁵R⁶NC₁-C₄alkyl-, —NHC(O)C₁-C₄alkyl, —NHCONR⁵R⁶, —NHSO₂C₁-C₄alkyl, —NHSO₂NR⁵R⁶, and R⁹; R⁵ is selected from the group consisting of: hydrogen, C₁-C₄ alkyl, C₃-C₇cycloalkyl, —C₁-C₃ alkyl C₃-C₇cycloalkyl, phenyl, and —C₁-C₃ alkylphenyl; R⁶ is hydrogen, C₁-C₄ alkyl, C₃-C₇ cycloalkyl, or —C₁-C₃ alkyl C₃-C₇ cycloalkyl; or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or C₁-C₄ alkyl; R⁹ is a 5- or 6-membered heteroaryl ring containing 1 to 4 heteroatoms selected from oxygen, nitrogen, and sulfur, which is optionally substituted with 1 or 2 substituents selected from halogen, C₁-C₄alkyl, CF₃, C₁-C₄alkoxy, and —NR⁵R⁶; R⁴ is oxo, halogen or C₁-C₆alkyl; R¹ is selected from the group consisting of: phenyl, 5- or 6-membered heteroaryl, napthyl, and 9- or 10-membered heterocyclyl, wherein said phenyl, 5- or 6-membered heteroaryl, napthyl, or 9- or 10-membered heterocyclyl, is optionally substituted with from 1 to 4 substituents independently selected from halogen, C₁-C₄ alkylhalogen, optionally substituted C₁-C₄ alkyl, —CF₃, —C₃-C₇cycloalkyl, —C(O)C₁-C₄ alkyl, —C(O)C₃-C₇cycloalkyl, —CO(phenyl), —C₁-C₄(═O)OH, —C(═O)OC₁-C₄ alkyl, —CONR⁵R⁶, phenyl, —SO₂C₁-C₄ alkyl, —SO₂NR⁵R⁶, cyano, oxo, hydroxyl, C₁-C₄ alkoxy, C₃-C₇ cycloalkoxy, hydroxy C₁-C₄ alkyl-, C₁-C₄ alkoxy C₁-C₄ alkyl-, —OCF₃, —NR⁵R⁶, R⁵R⁶NC₁-C₄ alkyl-, —NR⁶C(O)C₁-C₄ alkyl, —NR⁶C(O)C₃-C₇ cycloalkyl, —NR⁶CONR⁵R⁶, —NR⁶SO₂C₁-C₄alkyl, —NR⁶SO₂NR⁵R⁶, —NR⁶C(O)H, tetrazolyl, —B(OH)₂, —SO₃H, and R⁹; each R² is independently C₁-C₆ alkyl, cyano, C₁-C₄ alkoxy, hydroxyl, —CF₃, or halogen; each R⁷ is independently H, C₁-C₃alkyl, —C₁-C₄ alkylhalogen, halogen, cyano, —CONR⁵R⁶, —C(═O)OC₁-C₄ alkyl, hydroxy C₁-C₄ alkyl-, and —C(═O)OH; n is 0, 1, 2, 3, or 4; m is 0, 1, 2, or 3; Y is C or N, provided that when one Y is N the other Y is C; or a pharmaceutically acceptable salt thereof.

In some embodiments, Cy is a phenyl, optionally substituted with from one to three groups selected from the group consisting of: C₁-C₆alkyl, cyano, C₁-C₄alkoxy, hydroxyl, —CF₃, and halogen; or a pharmaceutically acceptable salt thereof. In some embodiments, Cy is 5- or 6-membered heteroaryl, optionally substituted with one to two groups selected from the group consisting of: C₁-C₆alkyl, cyano, C₁-C₄alkoxy, hydroxyl, —CF₃, and halogen; or a pharmaceutically acceptable salt thereof. In some embodiments, Cy is 5-membered heteroaryl selected from the group consisting of

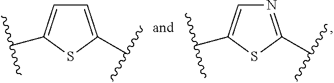 [SMILES: *c1ccc(*)s1 ; *c1cnc(*)s1]

which may be substituted with one to two groups selected from the group consisting C₁-C₆ alkyl, cyano, C₁-C₄ alkoxy, hydroxyl, —CF₃, and halogen; or a pharmaceutically acceptable salt thereof. In some embodiments, each R⁷ is H.

In some embodiments, the compound has the structure of Formula (XXV-B):

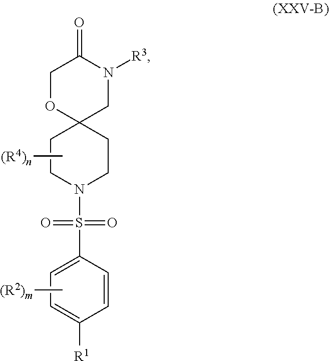

wherein R³ is selected from the group consisting of: C₁-C₆alkyl, C₃-C₇cycloalkyl, and C₄-C₆heterocycloalkyl, wherein said C₁-C₆alkyl, C₃-C₇cycloalkyl or C₄-C₆heterocycloalkyl is optionally substituted with from 1 to 6 substituents independently selected from the group of: halogen, C₁-C₄alkyl, C₁-C₄alkylhalogen, —CF₃, C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NHC(O)C₁-C₄alkyl, —NHCONR⁵R⁶, —NHSO₂C₁-C₄alkyl, —NHSO₂NR⁵R⁶, and R⁹; R⁵ is selected from the group consisting of: hydrogen, C₁-C₄alkyl, C₃-C₇cycloalkyl, —C₁-C₃alkylC₃-C₇cycloalkyl, phenyl, and —C₁-C₃alkylphenyl; R⁶ is hydrogen, C₁-C₄alkyl, C₃-C₇cycloalkyl, or —C₁-C₃alkyl C₃-C₇cycloalkyl; or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or C₁-C₄alkyl; R⁹ is a 5- or 6-membered heteroaryl ring containing 1 to 4 heteroatoms selected from oxygen, nitrogen, and sulfur, which is optionally substituted with 1 or 2 substituents selected from halogen, C₁-C₄alkyl, CF₃, C₁-C₄alkoxy, and —NR⁵R⁶; R⁴ is oxo, halogen or C₁-C₆alkyl; R¹ is selected from the group consisting of: phenyl, 5- or 6-membered heteroaryl, napthyl, and 9- or 10-membered heterocyclyl, wherein said phenyl, 5- or 6-membered heteroaryl, napthyl, 9- or 10-membered heterocyclyl, is optionally substituted with from 1 to 4 substituents independently selected from halogen, C₁-C₄alkylhalogen, optionally substituted C₁-C₄alkyl, —CF₃, —C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇ cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)C₃-C₇cycloalkyl, —NR⁶CONR⁵R⁶, —NR⁶SO₂OC₁-C₄alkyl, —NR⁶SO₂NR⁵R⁶, —NR⁶C(O)H, tetrazolyl, —B(OH)₂, —SO₃H, and R⁹; each R² is independently C₁-C₆alkyl, cyano, C₁-C₄alkoxy, hydroxyl, CF₃, or halogen; n is 0, 1, 2, 3, or 4; m is 0, 1, 2, or 3; or a pharmaceutically acceptable salt thereof.

In some embodiments, R¹ is phenyl optionally substituted with from 1 to 3 substituents independently selected from halogen, C₁-C₄alkylhalogen, optionally substituted C₁-C₄alkyl, —CF₃, —C₃-C₇ cycloalkyl, —C(O)C C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)C₃-C₇cycloalkyl, —NR⁶CONR⁵R⁶, —NR⁶SO₂C₁-C₄alkyl, —NR⁶SO₂NR⁵R⁶, —NR⁶C(O)H, tetrazolyl, —B(OH)₂, —SO₃H, and R⁹, wherein R⁵, R⁶, and R⁹ are defined as for Formula (XXV). In some embodiments, R¹ is selected from furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, and triazinyl, wherein said furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, and triazinyl are each optionally substituted with from 1 to 3 substituents independently selected from halogen, C₁-C₄alkylhalogen, optionally substituted C₁-C₄alkyl, —CF₃, —C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)C₃-C₇cycloalkyl, —NR⁶CONR⁵R⁶, —NR⁶SO₂C₁-C₄alkyl, —NR⁶SO₂NR⁵R⁶, —NR⁶C(O)H, tetrazolyl, —B(OH)₂, —SO₃H, and R⁹, wherein R⁵, R⁶, and R⁹ are defined as for Formula (XXV). In some embodiments, R¹ is napthyl optionally substituted with from 1 to 3 substituents independently selected from halogen, C₁-C₄alkylhalogen, optionally substituted C₁-C₄alkyl, —CF₃, —C₃-C₇cycloalkyl, —C(O)C C₄alkyl, —C(O)C₃-C₇cycloalkyl, CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)C₃-C₇cycloalkyl, —NR⁶CONR⁵R⁶, —NR⁶SO₂C₁-C₄alkyl, —NR⁶SO₂NR⁵R⁶, —NR⁶C(O)H, tetrazolyl, —B(OH)₂, —SO₃H, and R⁹, wherein R⁵, R⁶, and R⁹ are defined as for Formula (XXV). In some embodiments, R¹ is selected from benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, 1-H-indazolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, Cinnolinyl, phthalazinyl, quinazolinyl, 1,5-naphthyridinyl, 1,6-naphthyridinyl, 1,7-naphthyridinyl, 1,8-naphthyridinyl, and pteridinyl, wherein said benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, 1-H-indazolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, Cinnolinyl, phthalazinyl, quinazolinyl, 1,5-naphthyridinyl, 1,6-naphthyridinyl, 1,7-naphthyridinyl, 1,8-naphthyridinyl, and pteridinyl are each optionally substituted with from 1 to 3 substituents independently selected from halogen, C₁-C₄alkylhalogen, optionally substituted C₁-C₄alkyl, —CF₃, —C₃-C₇cycloalkyl, —C(O)C C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)C₃-C₇cycloalkyl, —NR⁶CONR⁵R⁶, —NR⁶SO₂C₁-C₄alkyl, —NR⁶SO₂NR⁵R⁶, —NR⁶C(O)H, tetrazolyl, —B(OH)₂, —SO₃H, and R⁹, wherein R⁵, R⁶, and R⁹ are defined as for Formula (XXV). In some embodiments, R³ is C₁-C₆alkyl or C₃-C₇ cycycloalkyl.

In some embodiments, the compound is 4-methyl-9-{[4-(7-quinolinyl)phenyl]sulfonyl}-1-oxa-4,9-diazaspiro[5.5]undecan-3-one; 9-{[4-(1H-indol-6-yl)phenyl]sulfonyl}-4-methyl-1-oxa-4,9-diazaspiro[5.5]undecan-3-one; 9-{[4-(1-benzofuran-5-yl)phenyl]sulfonyl}-4-ethyl-1-oxa-4,9-diazaspiro[5.5]undecan-3-one; 4-ethyl-9-{[4-(7-quinolinyl)phenyl]sulfonyl}-1-oxa-4,9-diazaspiro[5.5]undecan-3-one; 4-(1-methylethyl)-9-{[4-(7-quinolinyl)phenyl]sulfonyl}-1-oxa-4,9-diazaspiro[5.5]undecan-3-one; 9-{[4-(7-quinolinyl)phenyl]sulfonyl}-4-(2,2,2-trifluoroethyl)-1-oxa-4,9-diazaspiro[5.5]undecan-3-one; 4-(2-furanylmethyl)-9-{[4-(7-quinolinyl)phenyl]sulfonyl}-1-oxa-4,9-diazaspiro[5.5]undecan-3-one; 4-[2-(methyloxy)ethyl]-9-{[4-(7-quinolinyl)phenyl]sulfonyl}-1-oxa-4,9-diazaspiro[5.5]undecan-3-one; 4-(phenylmethyl)-9-{[4-(7-quinolinyl)phenyl]sulfonyl}-1-oxa-4,9-diazaspiro[5.5]undecan-3-one; 4-(1,1-dimethylethyl)-9-{[4-(7-quinolinyl)phenyl]sulfonyl}-1-oxa-4,9-diazaspiro[5.5]undecan-3-one; 4-(1-methylcyclopropyl)-9-{[4-(7-quinolinyl)phenyl]sulfonyl}-1-oxa-4,9-diazaspiro[5.5]undecan-3-one; 4-cyclobutyl-9-{[4-(7-quinolinyl)phenyl]sulfonyl}-1-oxa-4,9-diazaspiro[5.5]undecan-3-one; 9-(4-biphenylylsulfonyl)-4-cyclopropyl-1-oxa-4,9-diazaspiro[5.5]undecan-3-one; 4-cyclopropyl-9-{[4-(1H-indol-6-yl)phenyl]sulfonyl}-1-oxa-4,9-diazaspiro[5.5]undecan-3-one; 4′-[(4-cyclopropyl-3-oxo-1-oxa-4,9-diazaspiro[5.5]undec-9-yl)sulfonyl]-4-biphenylcarbonitrile; 4-cyclopropyl-9-[(4′-fluoro-4-biphenylyl)sulfonyl]-1-oxa-4,9-diazaspiro[5.5]undecan-3-one; 4-cyclopropyl-9-{[4-(1H-indazol-6-yl)phenyl]sulfonyl}-1-oxa-4,9-diazaspiro[5.5]undecan-3-one; 4-cyclopropyl-9-{[4-(1H-indol-5-yl)phenyl]sulfonyl}-1-oxa-4,9-diazaspiro[5.5]undecan-3-one; 9-{[4-(1-benzofuran-5-yl)phenyl]sulfonyl}-4-cyclopropyl-1-oxa-4,9-diazaspiro[5.5]undecan-3-one; 4′-[(4-cyclopropyl-3-oxo-1-oxa-4,9-diazaspiro[5.5]undec-9-yl)sulfonyl]-3-methyl-4-biphenylcarbonitrile; 4-cyclopropyl-9-{[2-fluoro-4-(1H-indol-6-yl)phenyl]sulfonyl}-1-oxa-4,9-diazaspiro[5.5]undecan-3-one; 4′-[(4-cyclopropyl-3-oxo-1-oxa-4,9-diazaspiro[5.5]undec-9-yl)sulfonyl]-3′-fluoro-4-biphenylcarbonitrile; 4-cyclopropyl-9-[(3,4′-difluoro-4-biphenylyl)sulfonyl]-1-oxa-4,9-diazaspiro[5.5]undecan-3-one; 4-cyclopropyl-9-{[4-(1-methyl-2-oxo-1,2-dihydro-6-quinolinyl)phenyl]sulfonyl}-1-oxa-4,9-diazaspiro[5.5]undecan-3-one; 4-cyclopropyl-9-{[4-(1-methyl-2,3-dihydro-1H-indol-5-yl)phenyl]sulfonyl}-1-oxa-4,9-diazaspiro[5.5]undecan-3-one; 9-{[4-(1-benzofuran-2-yl)phenyl]sulfonyl}-4-cyclopropyl-1-oxa-4,9-diazaspiro[5.5]undecan-3-one; 9-{[4-(1-benzothien-2-yl)phenyl]sulfonyl}-4-cyclopropyl-1-oxa-4,9-diazaspiro[5.5]undecan-3-one; 9-{[4-(1,3-benzoxazol-2-yl)phenyl]sulfonyl}-4-cyclopropyl-1-oxa-4,9-diazaspiro[5.5]undecan-3-one; 4-cyclopropyl-9-{[4-(7-quinolinyl)phenyl]sulfonyl}-1-oxa-4,9-diazaspiro[5.5]undecan-3-one; 4-cyclopropyl-9-{[4-(7-quinolinyl)phenyl]sulfonyl}-1,4,9-triazaspiro[5.5]undecan-3-one; 4-cyclopropyl-1-methyl-9-{[4-(7-quinolinyl)phenyl]sulfonyl}-1,4,9-triazaspiro[5.5]undecan-3-one; 4-cyclopropyl-8-methyl-9-{[4-(7-quinolinyl)phenyl]sulfonyl}-1-oxa-4,9-diazaspiro[5.5]undecan-3-one; 4-cyclopropyl-7-methyl-9-{[4-(7-quinolinyl)phenyl]sulfonyl}-1-oxa-4,9-diazaspiro[5.5]undecan-3-one; 9-[(4-imidazo[1,2-a]pyridin-7-ylphenyl)sulfonyl]-4-(1-methylcyclopropyl)-1-oxa-4,9-diazaspiro[5.5]undecan-3-one; 4-cyclopropyl-7-fluoro-9-[(4-imidazo[1,2-a]pyridin-7-ylphenyl)sulfonyl]-1-oxa-4,9-diazaspiro[5.5]undecan-3-one; 4-cyclopropyl-7-fluoro-9-{[4-(7-quinolinyl)phenyl]sulfonyl}-1-oxa-4,9-diazaspiro[5.5]undecan-3-one; 4-cyclopropyl-9-{[3-fluoro-4-(1H-indol-6-yl)phenyl]sulfonyl}-1-oxa-4,9-diazaspiro[5.5]undecan-3-one; 4-cyclopropyl-9-{[3-fluoro-4-(7-quinolinyl)phenyl]sulfonyl}-1-oxa-4,9-diazaspiro[5.5]undecan-3-one; 9-{[3-chloro-4-(1H-indol-6-yl)phenyl]sulfonyl}-4-cyclopropyl-1-oxa-4,9-diazaspiro[5.5]undecan-3-one; 9-{[3-chloro-4-(7-quinolinyl)phenyl]sulfonyl}-4-cyclopropyl-1-oxa-4,9-diazaspiro[5.5]undecan-3-one; 4-cyclopropyl-9-{[4-(1H-indol-6-yl)-3-methylphenyl]sulfonyl}-1-oxa-4,9-diazaspiro[5.5]undecan-3-one; 4-cyclopropyl-9-{[3-methyl-4-(7-quinolinyl)phenyl]sulfonyl}-1-oxa-4,9-diazaspiro[5.5]undecan-3-one; 4-cyclopropyl-9-{[2,5-difluoro-4-(1H-indol-6-yl)phenyl]sulfonyl}-1-oxa-4,9-diazaspiro[5.5]undecan-3-one; 4-cyclopropyl-9-{[2,5-difluoro-4-(7-quinolinyl)phenyl]sulfonyl}-1-oxa-4,9-diazaspiro[5.5]undecan-3-one; 4-cyclopropyl-9-{[3-(methyloxy)-4-(7-quinolinyl)phenyl]sulfonyl}-1-oxa-4,9-diazaspiro[5.5]undecan-3-one; 4-cyclopropyl-9-{[4-(1H-indol-6-yl)-3-(methyloxy)phenyl]sulfonyl}-1-oxa-4,9-diazaspiro[5.5]undecan-3-one; 4-cyclopropyl-9-{[5-(7-quinolinyl)-2-pyridinyl]sulfonyl}-1-oxa-4,9-diazaspiro[5.5]undecan-3-one; 4-cyclopropyl-9-{[5-(1H-indol-6-yl)-2-pyridinyl]sulfonyl}-1-oxa-4,9-diazaspiro[5.5]undecan-3-one; 4-cyclopropyl-9-{[4-(7-quinolinyl)-3-(trifluoromethyl)phenyl]sulfonyl}-1-oxa-4,9-diazaspiro[5.5]undecan-3-one; 4-cyclopropyl-9-{[2-methyl-4-(7-quinolinyl)phenyl]sulfonyl}-1-oxa-4,9-diazaspiro[5.5]undecan-3-one; 4-cyclopropyl-9-{[4-(1H-indol-6-yl)-2-methylphenyl]sulfonyl}-1-oxa-4,9-diazaspiro[5.5]undecan-3-one; 9-{[2-chloro-4-(1H-indol-6-yl)phenyl]sulfonyl}-4-cyclopropyl-1-oxa-4,9-diazaspiro[5.5]undecan-3-one; 4-cyclopropyl-9-{[5-(1H-indol-6-yl)-2-thienyl]sulfonyl}-1-oxa-4,9-diazaspiro[5.5]undecan-3-one; 9-{[2-chloro-4-(7-quinolinyl)phenyl]sulfonyl}-4-cyclopropyl-1-oxa-4,9-diazaspiro[5.5]undecan-3-one; 4-cyclopropyl-9-{[2-fluoro-4-(7-quinolinyl)phenyl]sulfonyl}-1-oxa-4,9-diazaspiro[5.5]undecan-3-one; 4-cyclopropyl-9-{[5-(7-quinolinyl)-2-thienyl]sulfonyl}-1-oxa-4,9-diazaspiro[5.5]undecan-3-one; 4-cyclopropyl-9-{[6-(7-quinolinyl)-3-pyridinyl]sulfonyl}-1-oxa-4,9-diazaspiro[5.5]undecan-3-one; 4-cyclopropyl-9-{[6-(1H-indol-6-yl)-3-pyridinyl] sulfonyl}-1-oxa-4,9-diazaspiro[5.5]undecan-3-one; 4-cyclopropyl-9-{[2,3-dimethyl-4-(7-quinolinyl)phenyl]sulfonyl}-1-oxa-4,9-diazaspiro[5.5]undecan-3-one; 9-{[3-chloro-2-fluoro-4-(7-quinolinyl)phenyl]sulfonyl}-4-cyclopropyl-1-oxa-4,9-diazaspiro[5.5]undecan-3-one; 9-{[3-chloro-2-fluoro-4-(1H-indol-6-yl)phenyl]sulfonyl}-4-cyclopropyl-1-oxa-4,9-diazaspiro[5.5]undecan-3-one; 4-cyclopropyl-9-{[3,5-difluoro-4-(1H-indol-6-yl)phenyl]sulfonyl}-1-oxa-4,9-diazaspiro[5.5]undecan-3-one; 4-cyclopropyl-9-{[3,5-difluoro-4-(7-quinolinyl)phenyl]sulfonyl}-1-oxa-4,9-diazaspiro[5.5]undecan-3-one; 4-cyclopropyl-9-{[4-methyl-5-(7-quinolinyl)-2-thienyl]sulfonyl}-1-oxa-4,9-diazaspiro[5.5]undecan-3-one; 4-cyclopropyl-9-{[2-(7-quinolinyl)-1,3-thiazol-5-yl]sulfonyl}-1-oxa-4,9-diazaspiro[5.5]undecan-3-one; 4-cyclopropyl-9-{[2-fluoro-4-(1H-indol-5-yl)phenyl]sulfonyl}-1-oxa-4,9-diazaspiro[5.5]undecan-3-one; 9-{[4-(1-benzofuran-5-yl)-2-fluorophenyl]sulfonyl}-4-cyclopropyl-1-oxa-4,9-diazaspiro[5.5]undecan-3-one; 4-cyclopropyl-9-{[2-fluoro-4-(1H-indazol-5-yl)phenyl]sulfonyl}-1-oxa-4,9-diazaspiro[5.5]undecan-3-one; 4-cyclopropyl-9-{[2-fluoro-4-(6-quinolinyl)phenyl]sulfonyl}-1-oxa-4,9-diazaspiro[5.5]undecan-3-on; 4-cyclopropyl-9-[(2′,4′-dichloro-3-fluoro-4-biphenylyl)sulfonyl]-1-oxa-4,9-diazaspiro[5.5]undecan-3-one; 4-cyclopropyl-9-{[3-fluoro-4′-(methyloxy)-4-biphenylyl]sulfonyl}-1-oxa-4,9-diazaspiro[5.5]undecan-3-one; 9-{[4-(1,3-benzothiazol-5-yl)-2-fluorophenyl]sulfonyl}-4-cyclopropyl-1-oxa-4,9-diazaspiro[5.5]undecan-3-one; {4′-[4-cyclopropyl-3-oxo-1-oxa-4,9-diazaspiro[5.5]undec-9-yl)sulfonyl]-3′-fluoro-4-hydroxy-3-biphenylyl}formamide; 4-cyclopropyl-9-{[2-fluoro-4-(5-quinolinyl)phenyl]sulfonyl}-1-oxa-4,9-diazaspiro[5.5]undecan-3-one; 4-cyclopropyl-9-{[3-fluoro-4′-(1H-pyrazol-1-yl)-4-biphenylyl]sulfonyl}-1-oxa-4,9-diazaspiro[5.5]undecan-3-one; 9-{[4-(1,3-benzoxazol-5-yl)-2-fluorophenyl]sulfonyl}-4-cyclopropyl-1-oxa-4,9-diazaspiro[5.5]undecan-3-one; 4-cyclopropyl-9-{[3′-(1H-pyrazol-5-yl)-4-biphenylyl]sulfonyl}-1-oxa-4,9-diazaspiro[5.5]undecan-3-one; 4-cyclopropyl-9-{[4′-(1H-pyrazol-5-yl)-4-biphenylyl]sulfonyl}-1-oxa-4,9-diazaspiro[5.5]undecan-3-one; 4-cyclopropyl-9-{[4-(7-isoquinolinyl)phenyl]sulfonyl}-1-oxa-4,9-diazaspiro[5.5]undecan-3-one; 4-cyclopropyl-9-{[4-(7-quinazolinyl)phenyl]sulfonyl}-1-oxa-4,9-diazaspiro[5.5]undecan-3-one; N′-{4′-[(4-cyclopropyl-3-oxo-1-oxa-4,9-diazaspiro[5.5]undec-9-yl)sulfonyl]-3 biphenylyl}-N,N-dimethylsulfamide; 4-cyclopropyl-9-{[4-(6-isoquinolinyl)phenyl]sulfonyl}-1-oxa-4,9-diazaspiro[5.5]undecan-3-one; 4-cyclopropyl-9-{[4-(3-quinolinyl)phenyl]sulfonyl}-1-oxa-4,9-diazaspiro[5.5]undecan-3-one; 4-cyclopropyl-9-{[4-(2-naphthalenyl)phenyl]sulfonyl}-1-oxa-4,9-diazaspiro[5.5]undecan-3-one; 4-cyclopropyl-9-{[4-(2-methyl-1,3-benzothiazol-5-yl)phenyl]sulfonyl}-1-oxa-diazaspiro[5.5]undecan-3-one; 4-cyclopropyl-9-({4-[4-(ethyloxy)-7-quinolinyl]phenyl}sulfonyl)-1-oxa-4,9-diazaspiro[5.5]undecan-3-one; 4-cyclopropyl-9-({4-[4-(methyloxy)-7-quinolinyl]phenyl}sulfonyl)-1-oxa-4,9-diazaspiro[5.5]undecan-3-one; 4-cyclopropyl-9-[(4-imidazo[1,2-a]pyridin-7-ylphenyl)sulfonyl]-1-oxa-4,9-diazaspiro[5.5]undecan-3-one; 9-{[4-(3-amino-1H-indazol-6-yl)phenyl]sulfonyl}-4-cyclopropyl-1-oxa-4,9-diazaspiro[5.5]undecan-3-one; 9-{[4-(3-amino-1H-indazol-5-yl)phenyl]sulfonyl}-4-cyclopropyl-1-oxa-4,9-diazaspiro[5.5]undecan-3-one; 9-{[4-(2-amino-4-pyridinyl)phenyl]sulfonyl}-4-cyclopropyl-1-oxa-4,9-diazaspiro[5.5]undecan-3-one; 4-cyclopropyl-9-{[4-(4-quinolinyl)phenyl]sulfonyl}-1-oxa-4,9-diazaspiro[5.5]undecan-3-one; 4-cyclopropyl-9-{[4-(1-methyl-1H-indol-6-yl)phenyl]sulfonyl}-1-oxa-4,9-diazaspiro[5.5]undecan-3-one; 4-cyclopropyl-9-{[4-(1-methyl-1H-indol-4-yl)phenyl]sulfonyl}-1-oxa-4,9-diazaspiro[5.5]undecan-3-one; 4-cyclopropyl-9-({4-[4-(methylamino)-7-quinolinyl]phenyl} sulfonyl)-1-oxa-4 diazaspiro[5.5]undecan-3-one; 4-cyclopropyl-9-{[4-(4-methyl-7-quinolinyl)phenyl]sulfonyl}-1-oxa-4,9-diazaspiro[5.5]undecan-3-one; 4-cyclopropyl-9-[(4-imidazo[1,2-a]pyridin-6-ylphenyl)sulfonyl]-1-oxa-4,9-diazaspiro[5.5]undecan-3-one; 9-{[4-(7-cinnolinyl)phenyl]sulfonyl}-4-cyclopropyl-1-oxa-4,9-diazaspiro[5.5]undecan-3-one; 4-cyclopropyl-9-[(4-imidazo[1,2-b]pyridazin-6-ylphenyl)sulfonyl]-1-oxa-4,9-diazaspiro[5.5]undecan-3-one; 9-{[4-(3-amino-1-methyl-1H-indazol-5-yl)phenyl]sulfonyl}-4-cyclopropyl-1-oxa-4,diazaspiro[5.5]undecan-3-one; N-(5-{4-[(4-cyclopropyl-3-oxo-1-oxa-4,9-diazaspiro[5.5]undec-9-yl)sulfonyl]phenyl}-1-methyl-1H-indazol-3-yl)methanesulfonamide; 9-{[4-(3-amino-1-methyl-1H-indazol-6-yl)phenyl]sulfonyl}-4-cyclopropyl-1-oxa-4,diazaspiro[5.5]undecan-3-one; N-(6-{4-[(4-cyclopropyl-3-oxo-1-oxa-4,9-diazaspiro[5.5]undec-9-yl)sulfonyl]phenyl}-1H-indazol-3-yl)-N′-methylurea; 4-cyclopropyl-9-((4-(8-methylquinolin-7-yl)phenyl)sulfonyl)-1-oxa-4,9-diazaspiro[5.5]undecan-3-one; 4-cyclopropyl-9-((4-(8-fluoroquinolin-7-yl)phenyl)sulfonyl)-1-oxa-4,9-diazaspiro[5.5]undecan-3-one; 1-(3-oxo-9-{[4-(7-quinolinyl)phenyl]sulfonyl}-1-oxa-4,9-diazaspiro[5.5]undec-4-yl)cyclopropanecarboxamide; 4-(1-methylcyclobutyl)-9-{[4-(7-quinolinyl)phenyl]sulfonyl}-1-oxa-4,9-diazaspiro[5.5]undecan-3-one; 1-(3-oxo-9-{[4-(7-quinolinyl)phenyl]sulfonyl}-1-oxa-4,9-diazaspiro[5.5]undec-4-yl)cyclopropanecarbonitrile; 4-(3-oxetanyl)-9-{[4-(7-quinolinyl)phenyl]sulfonyl}-1-oxa-4,9-diazaspiro[5.5]undecan-3-one; 4-[1-(hydroxymethyl)cyclopropyl]-9-{[4-(7-quinolinyl)phenyl]sulfonyl}-1-oxa-4,9-diazaspiro[5.5]undecan-3-one; 4-{1-[(methyloxy)methyl]cyclopropyl}-9-{[4-(7-quinolinyl)phenyl]sulfonyl}-1-oxa 4,9-diazaspiro[5.5]undecan-3-one; 4-[1-(hydroxymethyl)cyclopropyl]-9-({4-[3-(methyloxy)-7-quinolinyljphenyl} sulfonyl)-1-oxa-4,9-diazaspiro[5.5]undecan-3-one; 4-cyclopropyl-9-((4-(8-methoxyquinolin-7-yl)phenyl)sulfonyl)-1-oxa-4,9-diazaspiro[5.5]undecan-3-one; 4-cyclopropyl-9-((4-(8-hydroxyquinolin-7-yl)phenyl)sulfonyl)-1-oxa-4,9-diazaspiro[5.5]undecan-3-one; 4-cyclopropyl-9-{[4-(7-quinolinyl)phenyl]sulfonyl}-1-oxa-4,9-diazaspiro[5.5]undecan-3-one-d₄; 4-cyclopropyl-9-((4-(6-fluoronaphthalen-2-yl)phenyl)sulfonyl)-1-oxa-4,9-diazaspiro[5.5]undecan-3-one; 4-cyclopropyl-9-((4-(8-fluoronaphthalen-2-yl)phenyl)sulfonyl)-1-oxa-4,9-diazaspiro[5.5]undecan-3-one; 4-ethyl-9-((4-(3-methoxyquinolin-7-yl)phenyl)sulfonyl)-1-oxa-4,9-diazaspiro[5.5]undecan-3-one; 4-isopropyl-9-((4-(3-methoxyquinolin-7-yl)phenyl)sulfonyl)-1-oxa-4,9-diazaspiro[5.5]undecan-3-one; 4-ethyl-9-((4-(3-fluoroquinolin-7-yl)phenyl)sulfonyl)-1-oxa-4,9-diazaspiro[5.5]undecan-3-one; 9-((4-(3-fluoroquinolin-7-yl)phenyl)sulfonyl)-4-isopropyl-1-oxa-4,9-diazaspiro[5.5]undecan-3-one; 9-((4-(3-methoxyquinolin-7-yl)phenyl)sulfonyl)-4-(1-methylcyclopropyl)-1-oxa-4 diazaspiro[5.5]undecan-3-one; 9-((4-(3-fluoroquinolin-7-yl)phenyl)sulfonyl)-4-(1-methylcyclopropyl)-1-oxa-4,9-diazaspiro[5.5]undecan-3-one; 4′-[(4-cyclopropyl-3-oxo-1-oxa-4,9-diazaspiro[5.5]undec-9-yl)sulfonyl]-3-biphenylcarboxylic acid; 4-cyclopropyl-9-{[4′-(1H-tetrazol-5-yl)-4-biphenylyl]sulfonyl}-1-oxa-4,9-diazaspiro[5.5]undecan-3-one; {4′-[(4-cyclopropyl-3-oxo-1-oxa-4,9-diazaspiro[5.5]undec-9-yl)sulfonyl]-3-biphenylyl}boronic acid; 4′-[(4-cyclopropyl-3-oxo-1-oxa-4,9-diazaspiro[5.5]undec-9-yl)sulfonyl]-3-biphenylsulfonic acid; 2-cyclopropyl-9-{[4-(7-quinolinyl)phenyl]sulfonyl}-2,9-diazaspiro[5.5]undecan-3 one; ethyl 7-(4-((4-cyclopropyl-3-oxo-1-oxa-4,9-diazaspiro[5.5]undecan-9-yl)sulfonyl)phenyl)quinoline-3-carboxylate; 7-(4-((4-cyclopropyl-3-oxo-1-oxa-4,9-diazaspiro[5.5]undecan-9-yl)sulfonyl)phenyl)quinoline-3-carboxylic acid; 9-((4-(3-aminoquinolin-7-yl)phenyl)sulfonyl)-4-cyclopropyl-1-oxa-4,9-diazaspiro[5.5]undecan-3-one; N-(7-(4-((4-cyclopropyl-3-oxo-1-oxa-4,9-diazaspiro[5.5]undecan-9-yl)sulfonyl)phenyl)quinolin-3-yl)acetamide; 7-(4-((4-cyclopropyl-3-oxo-1-oxa-4,9-diazaspiro[5.5]undecan-9-yl)sulfonyl)phenyl)quinoline-3-carbonitrile; 4-cyclopropyl-9-((4-(3-methoxyquinolin-7-yl)phenyl)sulfonyl)-1-oxa-4,9-diazaspiro[5.5]undecan-3-one; 4-cyclopropyl-9-((4-(3-methylquinolin-7-yl)phenyl)sulfonyl)-1-oxa-4,9-diazaspiro[5.5]undecan-3-one; 9-((4-(3-chloroquinolin-7-yl)phenyl)sulfonyl)-4-cyclopropyl-1-oxa-4,9-diazaspiro[5.5]undecan-3-one; 4-cyclopropyl-9-((4-(3-hydroxyquinolin-7-yl)phenyl)sulfonyl)-1-oxa-4,9-diazaspiro[5.5]undecan-3-one; 7-(4-((4-cyclopropyl-3-oxo-1-oxa-4,9-diazaspiro[5.5]undecan-9-yl)sulfonyl)phenyl)quinoline-3-carboxamide; N-(7-(4-((4-cyclopropyl-3-oxo-1-oxa-4,9-diazaspiro[5.5]undecan-9-yl)sulfonyl)phenyl)quinolin-3-yl)cyclopropanecarboxamide; 2-(4-((4-cyclopropyl-3-oxo-1-oxa-4,9-diazaspiro[5.5]undecan-9-yl)sulfonyl)phenyl)thieno[3,2-b]pyridine-6-carboxamide; 4-cyclopropyl-9-((4-(3-fluoroquinolin-7-yl)phenyl)sulfonyl)-1-oxa-4,9-diazaspiro[5.5]undecan-3-one, or N-(7-(4-((4-cyclopropyl-3-oxo-1-oxa-4,9-diazaspiro[5.5]undecan-9-yl)sulfonyl)phenyl)quinolin-3-yl)methanesulfonamide, or pharmaceutically acceptable salts thereof.

In some embodiments, the compound is one of the following:

| | Compound |
|---|---|
| 1291 | 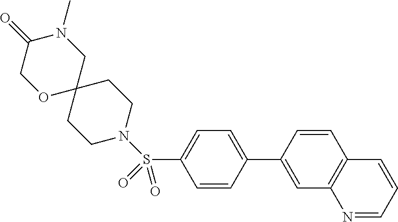 [SMILES: CN1CC2(CCN(S(=O)(=O)c3ccc(-c4ccc5cccnc5c4)cc3)CC2)OCC1=O] |
| 1292 | 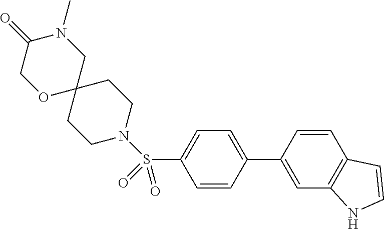 [SMILES: CN1CC2(CCN(S(=O)(=O)c3ccc(-c4ccc5cc[nH]c5c4)cc3)CC2)OCC1=O] |
| 1293 | 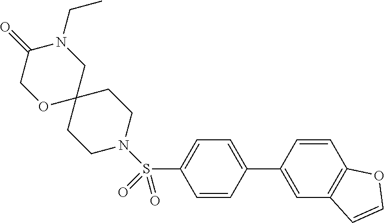 [SMILES: CCN1CC2(CCN(S(=O)(=O)c3ccc(-c4ccc5occc5c4)cc3)CC2)OCC1=O] |
| 1294 | 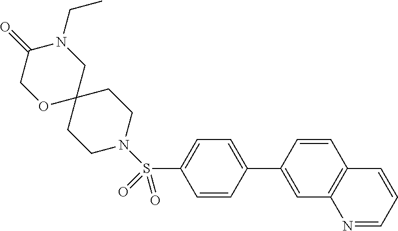 [SMILES: CCN1CC2(CCN(S(=O)(=O)c3ccc(-c4ccc5cccnc5c4)cc3)CC2)OCC1=O] |
| 1295 | 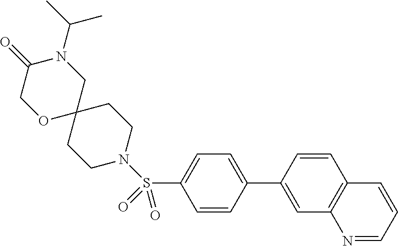 [SMILES: CC(C)N1CC2(CCN(S(=O)(=O)c3ccc(-c4ccc5cccnc5c4)cc3)CC2)OCC1=O] |
| 1296 | 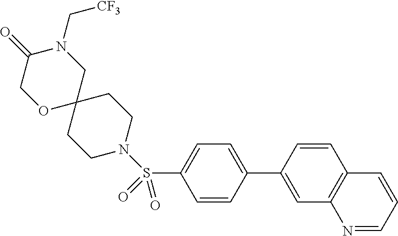 [SMILES: O=C1COC2(CCN(S(=O)(=O)c3ccc(-c4ccc5cccnc5c4)cc3)CC2)CN1CC(F)(F)F] |
| 1297 | 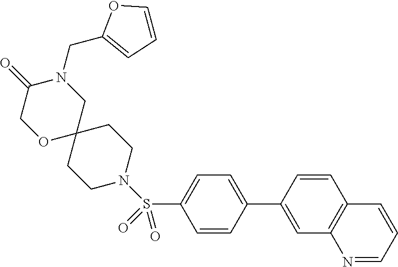 [SMILES: O=C1COC2(CCN(S(=O)(=O)c3ccc(-c4ccc5cccnc5c4)cc3)CC2)CN1Cc1ccco1] |
| 1298 | 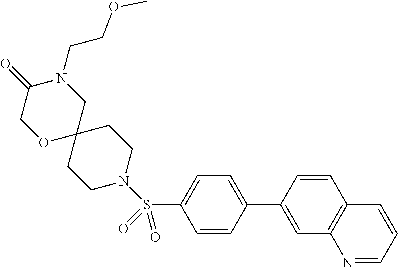 [SMILES: COCCN1CC2(CCN(S(=O)(=O)c3ccc(-c4ccc5cccnc5c4)cc3)CC2)OCC1=O] |
| 1299 | 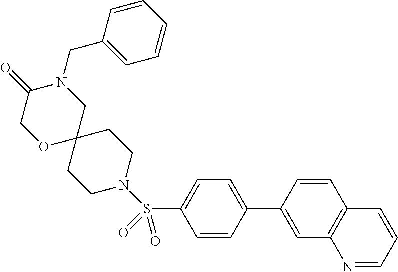 [SMILES: O=C1COC2(CCN(S(=O)(=O)c3ccc(-c4ccc5cccnc5c4)cc3)CC2)CN1Cc1ccccc1] |
| 1300 | 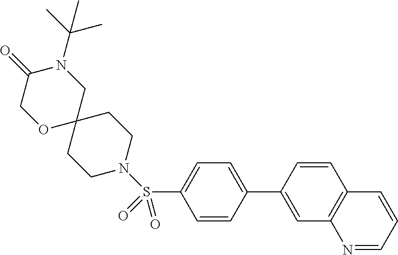 [SMILES: CC(C)(C)N1CC2(CCN(S(=O)(=O)c3ccc(-c4ccc5cccnc5c4)cc3)CC2)OCC1=O] |
| 1301 | 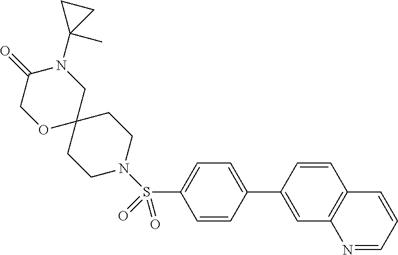 [SMILES: CC1(N2CC3(CCN(S(=O)(=O)c4ccc(-c5ccc6cccnc6c5)cc4)CC3)OCC2=O)CC1] |
| 1302 | 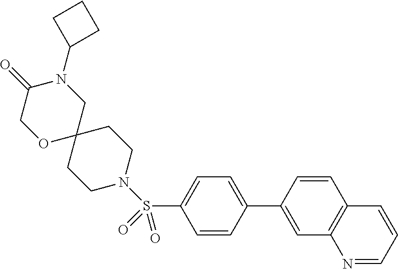 [SMILES: O=C1COC2(CCN(S(=O)(=O)c3ccc(-c4ccc5cccnc5c4)cc3)CC2)CN1C1CCC1] |
| 1303 | 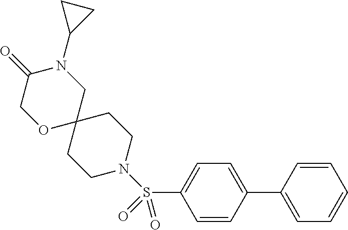 [SMILES: O=C1COC2(CCN(S(=O)(=O)c3ccc(-c4ccccc4)cc3)CC2)CN1C1CC1] |
| 1304 | 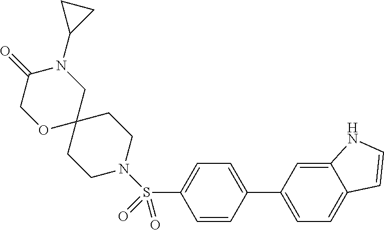 [SMILES: O=C1COC2(CCN(S(=O)(=O)c3ccc(-c4ccc5cc[nH]c5c4)cc3)CC2)CN1C1CC1] |
| 1305 | 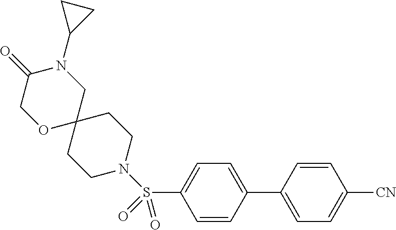 [SMILES: N#Cc1ccc(-c2ccc(S(=O)(=O)N3CCC4(CC3)CN(C3CC3)C(=O)CO4)cc2)cc1] |
| 1306 | 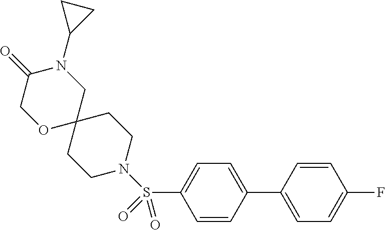 [SMILES: O=C1COC2(CCN(S(=O)(=O)c3ccc(-c4ccc(F)cc4)cc3)CC2)CN1C1CC1] |
| 1307 | 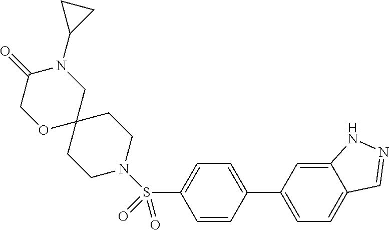 [SMILES: O=C1COC2(CCN(S(=O)(=O)c3ccc(-c4ccc5cn[nH]c5c4)cc3)CC2)CN1C1CC1] |
| 1308 | 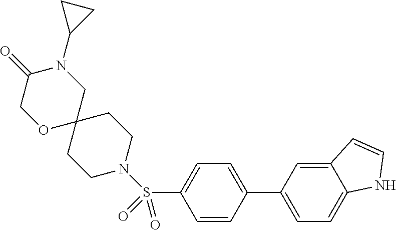 [SMILES: O=C1COC2(CCN(S(=O)(=O)c3ccc(-c4ccc5[nH]ccc5c4)cc3)CC2)CN1C1CC1] |
| 1309 | 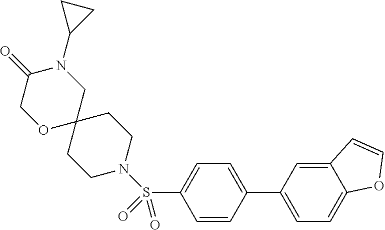 [SMILES: O=C1COC2(CCN(S(=O)(=O)c3ccc(-c4ccc5occc5c4)cc3)CC2)CN1C1CC1] |
| 1310 | 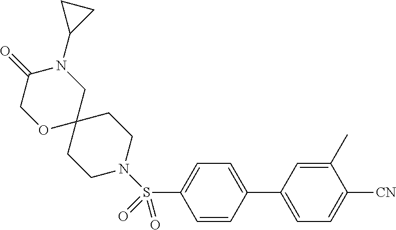 [SMILES: Cc1cc(-c2ccc(S(=O)(=O)N3CCC4(CC3)CN(C3CC3)C(=O)CO4)cc2)ccc1C#N] |
| 1311 | 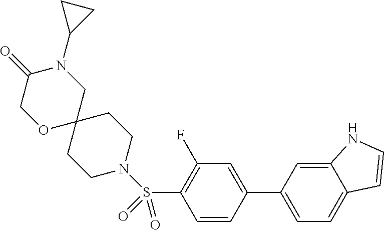 [SMILES: O=C1COC2(CCN(S(=O)(=O)c3ccc(-c4ccc5cc[nH]c5c4)cc3F)CC2)CN1C1CC1] |
| 1312 | 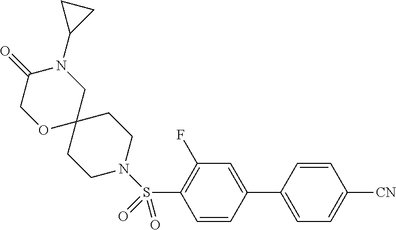 [SMILES: N#Cc1ccc(-c2ccc(S(=O)(=O)N3CCC4(CC3)CN(C3CC3)C(=O)CO4)c(F)c2)cc1] |
| 1313 | 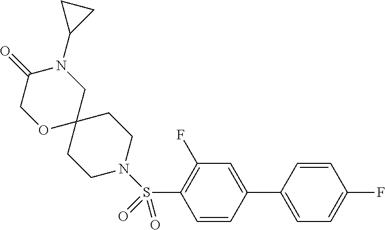 [SMILES: O=C1COC2(CCN(S(=O)(=O)c3ccc(-c4ccc(F)cc4)cc3F)CC2)CN1C1CC1] |
| 1314 | 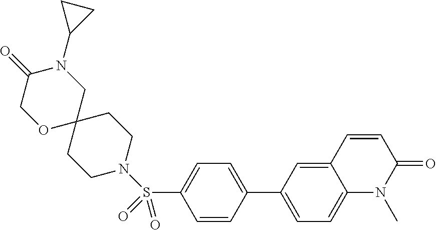 [SMILES: Cn1c(=O)ccc2cc(-c3ccc(S(=O)(=O)N4CCC5(CC4)CN(C4CC4)C(=O)CO5)cc3)ccc21] |
| 1315 | 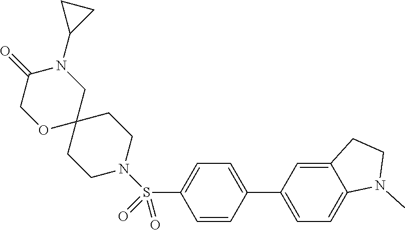 [SMILES: CN1CCc2cc(-c3ccc(S(=O)(=O)N4CCC5(CC4)CN(C4CC4)C(=O)CO5)cc3)ccc21] |
| 1316 | 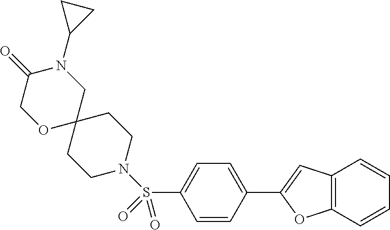 [SMILES: O=C1COC2(CCN(S(=O)(=O)c3ccc(-c4cc5ccccc5o4)cc3)CC2)CN1C1CC1] |
| 1317 | 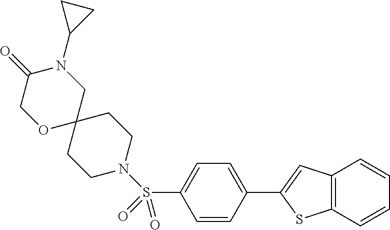 [SMILES: O=C1COC2(CCN(S(=O)(=O)c3ccc(-c4cc5ccccc5s4)cc3)CC2)CN1C1CC1] |
| 1318 | 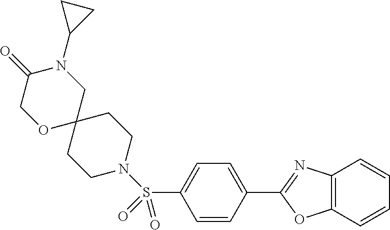 [SMILES: O=C1COC2(CCN(S(=O)(=O)c3ccc(-c4nc5ccccc5o4)cc3)CC2)CN1C1CC1] |
| 1319 | 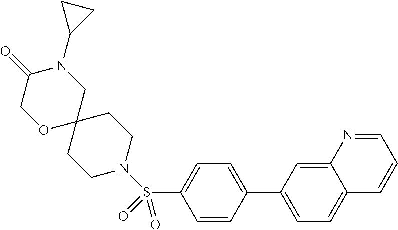 [SMILES: O=C1COC2(CCN(S(=O)(=O)c3ccc(-c4ccc5cccnc5c4)cc3)CC2)CN1C1CC1] |
| 1320 | 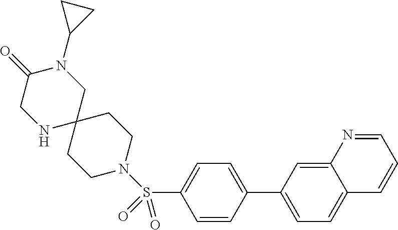 [SMILES: O=C1CNC2(CCN(S(=O)(=O)c3ccc(-c4ccc5cccnc5c4)cc3)CC2)CN1C1CC1] |
| 1321 | 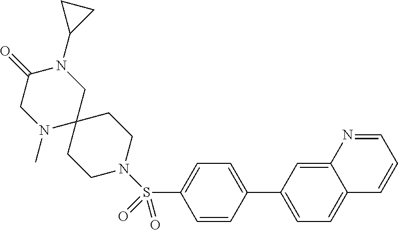 [SMILES: CN1CC(=O)N(C2CC2)CC12CCN(S(=O)(=O)c1ccc(-c3ccc4cccnc4c3)cc1)CC2] |
| 1322 | 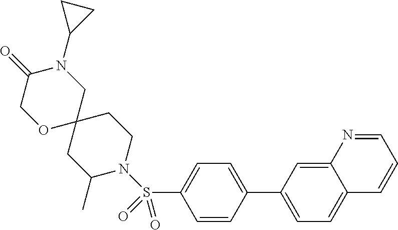 [SMILES: CC1CC2(CCN1S(=O)(=O)c1ccc(-c3ccc4cccnc4c3)cc1)CN(C1CC1)C(=O)CO2] |
| 1323 | 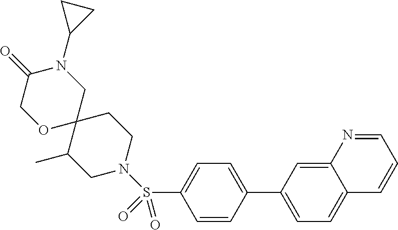 [SMILES: CC1CN(S(=O)(=O)c2ccc(-c3ccc4cccnc4c3)cc2)CCC12CN(C1CC1)C(=O)CO2] |
| 1324 | 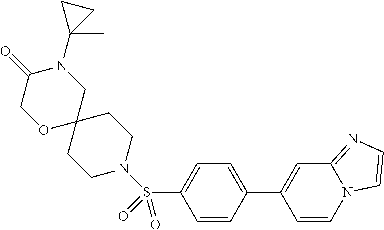 [SMILES: CC1(N2CC3(CCN(S(=O)(=O)c4ccc(-c5ccn6ccnc6c5)cc4)CC3)OCC2=O)CC1] |
| 1325 | 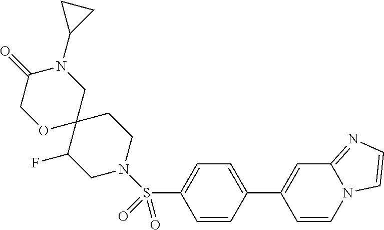 [SMILES: O=C1COC2(CCN(S(=O)(=O)c3ccc(-c4ccn5ccnc5c4)cc3)CC2F)CN1C1CC1] |
| 1326 | 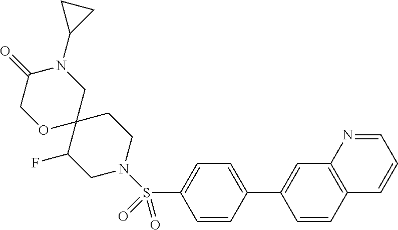 [SMILES: O=C1COC2(CCN(S(=O)(=O)c3ccc(-c4ccc5cccnc5c4)cc3)CC2F)CN1C1CC1] |
| 1327 | 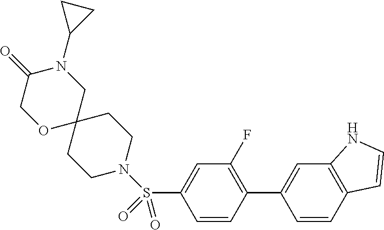 [SMILES: O=C1COC2(CCN(S(=O)(=O)c3ccc(-c4ccc5cc[nH]c5c4)c(F)c3)CC2)CN1C1CC1] |
| 1328 | 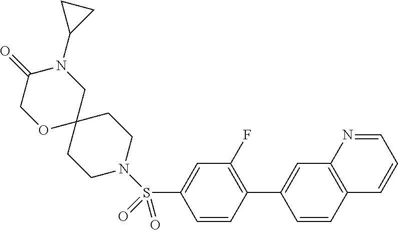 [SMILES: O=C1COC2(CCN(S(=O)(=O)c3ccc(-c4ccc5cccnc5c4)c(F)c3)CC2)CN1C1CC1] |
| 1329 | 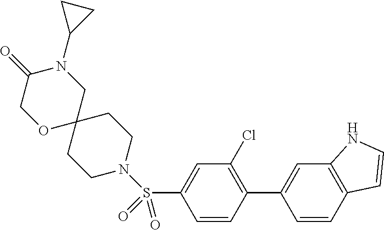 [SMILES: O=C1COC2(CCN(S(=O)(=O)c3ccc(-c4ccc5cc[nH]c5c4)c(Cl)c3)CC2)CN1C1CC1] |
| 1330 | 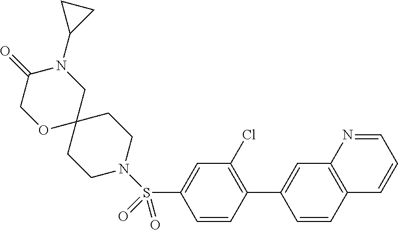 [SMILES: O=C1COC2(CCN(S(=O)(=O)c3ccc(-c4ccc5cccnc5c4)c(Cl)c3)CC2)CN1C1CC1] |
| 1331 | 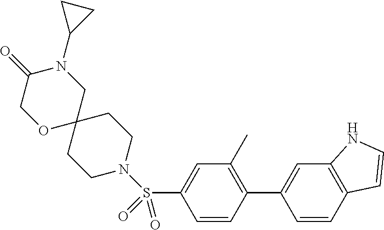 [SMILES: Cc1cc(S(=O)(=O)N2CCC3(CC2)CN(C2CC2)C(=O)CO3)ccc1-c1ccc2cc[nH]c2c1] |
| 1332 | 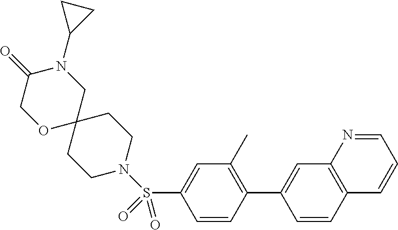 [SMILES: Cc1cc(S(=O)(=O)N2CCC3(CC2)CN(C2CC2)C(=O)CO3)ccc1-c1ccc2cccnc2c1] |
| 1333 | 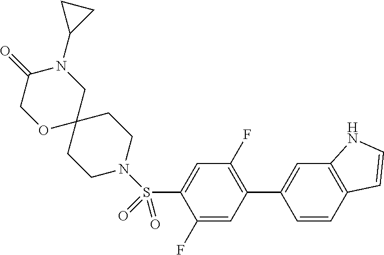 [SMILES: O=C1COC2(CCN(S(=O)(=O)c3cc(F)c(-c4ccc5cc[nH]c5c4)cc3F)CC2)CN1C1CC1] |
| 1334 | 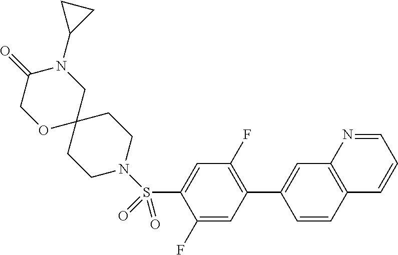 [SMILES: O=C1COC2(CCN(S(=O)(=O)c3cc(F)c(-c4ccc5cccnc5c4)cc3F)CC2)CN1C1CC1] |
| 1335 | 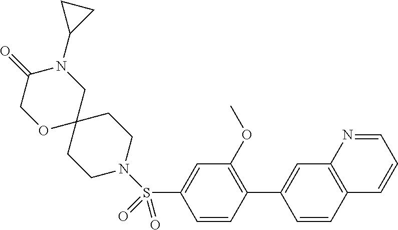 [SMILES: COc1cc(S(=O)(=O)N2CCC3(CC2)CN(C2CC2)C(=O)CO3)ccc1-c1ccc2cccnc2c1] |
| 1336 | 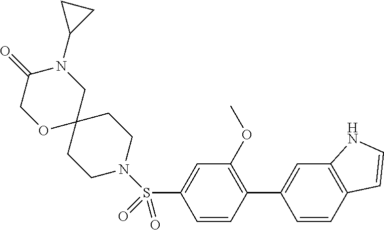 [SMILES: COc1cc(S(=O)(=O)N2CCC3(CC2)CN(C2CC2)C(=O)CO3)ccc1-c1ccc2cc[nH]c2c1] |
| 1337 | 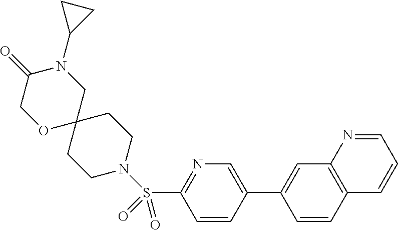 [SMILES: O=C1COC2(CCN(S(=O)(=O)c3ccc(-c4ccc5cccnc5c4)cn3)CC2)CN1C1CC1] |
| 1338 | 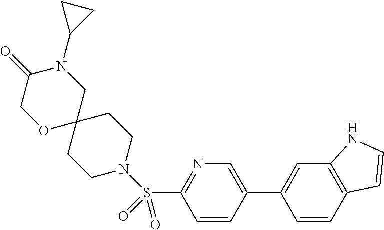 [SMILES: O=C1COC2(CCN(S(=O)(=O)c3ccc(-c4ccc5cc[nH]c5c4)cn3)CC2)CN1C1CC1] |
| 1339 | 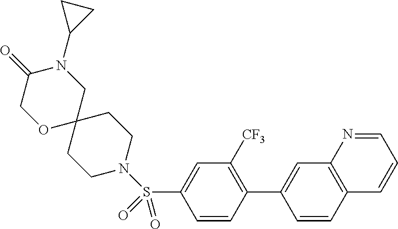 [SMILES: O=C1COC2(CCN(S(=O)(=O)c3ccc(-c4ccc5cccnc5c4)c(C(F)(F)F)c3)CC2)CN1C1CC1] |
| 1340 | 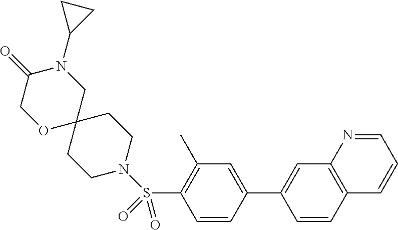 [SMILES: Cc1cc(-c2ccc3cccnc3c2)ccc1S(=O)(=O)N1CCC2(CC1)CN(C1CC1)C(=O)CO2] |
| 1341 | 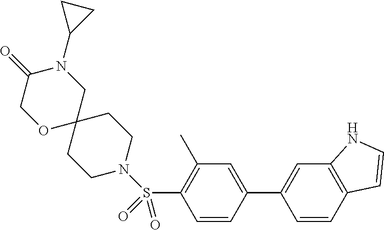 [SMILES: Cc1cc(-c2ccc3cc[nH]c3c2)ccc1S(=O)(=O)N1CCC2(CC1)CN(C1CC1)C(=O)CO2] |
| 1342 | 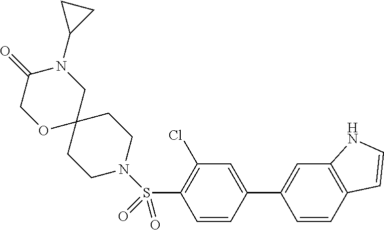 [SMILES: O=C1COC2(CCN(S(=O)(=O)c3ccc(-c4ccc5cc[nH]c5c4)cc3Cl)CC2)CN1C1CC1] |
| 1343 | 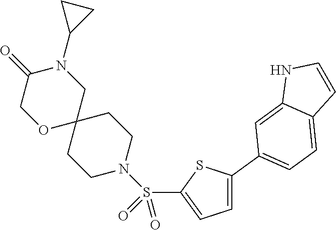 [SMILES: O=C1COC2(CCN(S(=O)(=O)c3ccc(-c4ccc5cc[nH]c5c4)s3)CC2)CN1C1CC1] |
| 1344 | 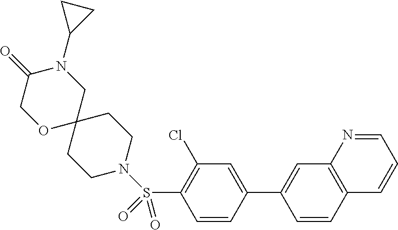 [SMILES: O=C1COC2(CCN(S(=O)(=O)c3ccc(-c4ccc5cccnc5c4)cc3Cl)CC2)CN1C1CC1] |
| 1345 | 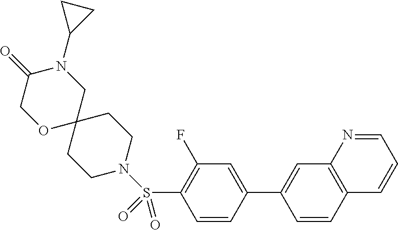 [SMILES: O=C1COC2(CCN(S(=O)(=O)c3ccc(-c4ccc5cccnc5c4)cc3F)CC2)CN1C1CC1] |
| 1346 | 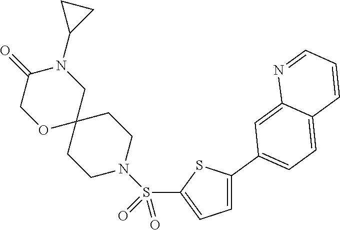 [SMILES: O=C1COC2(CCN(S(=O)(=O)c3ccc(-c4ccc5cccnc5c4)s3)CC2)CN1C1CC1] |
| 1347 | 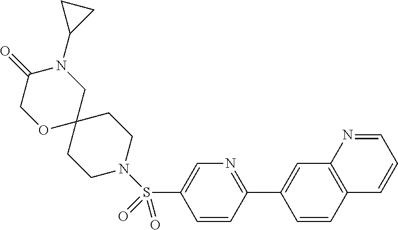 [SMILES: O=C1COC2(CCN(S(=O)(=O)c3ccc(-c4ccc5cccnc5c4)nc3)CC2)CN1C1CC1] |
| 1348 | 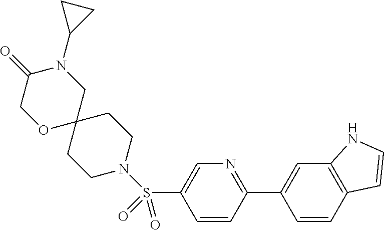 [SMILES: O=C1COC2(CCN(S(=O)(=O)c3ccc(-c4ccc5cc[nH]c5c4)nc3)CC2)CN1C1CC1] |
| 1349 | 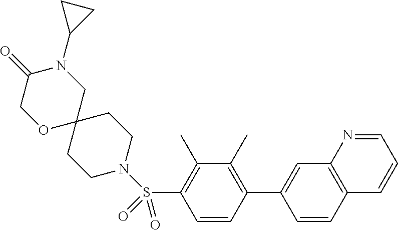 [SMILES: Cc1c(-c2ccc3cccnc3c2)ccc(S(=O)(=O)N2CCC3(CC2)CN(C2CC2)C(=O)CO3)c1C] |
| 1350 | 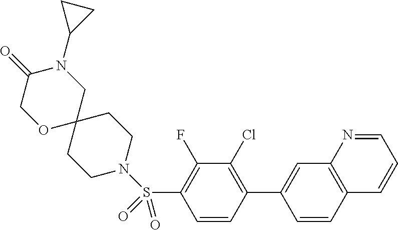 [SMILES: O=C1COC2(CCN(S(=O)(=O)c3ccc(-c4ccc5cccnc5c4)c(Cl)c3F)CC2)CN1C1CC1] |
| 1351 | 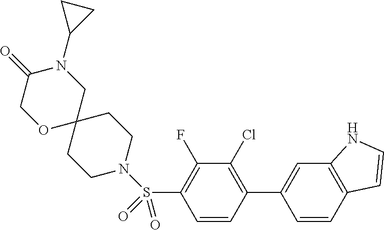 [SMILES: O=C1COC2(CCN(S(=O)(=O)c3ccc(-c4ccc5cc[nH]c5c4)c(Cl)c3F)CC2)CN1C1CC1] |
| 1352 | 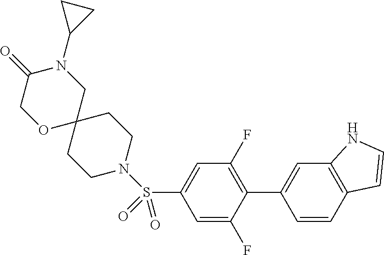 [SMILES: O=C1COC2(CCN(S(=O)(=O)c3cc(F)c(-c4ccc5cc[nH]c5c4)c(F)c3)CC2)CN1C1CC1] |
| 1353 | 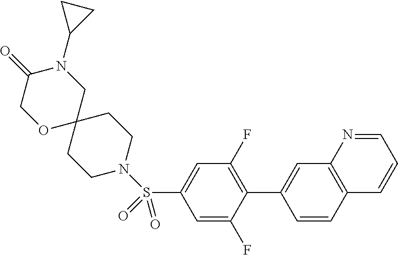 [SMILES: O=C1COC2(CCN(S(=O)(=O)c3cc(F)c(-c4ccc5cccnc5c4)c(F)c3)CC2)CN1C1CC1] |
| 1354 | 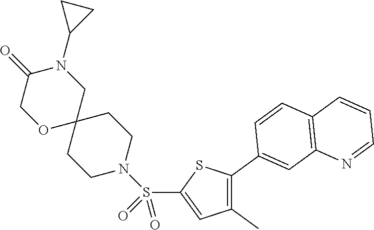 [SMILES: Cc1cc(S(=O)(=O)N2CCC3(CC2)CN(C2CC2)C(=O)CO3)sc1-c1ccc2cccnc2c1] |
| 1355 | 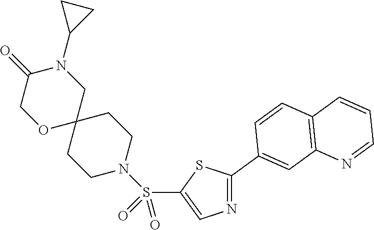 [SMILES: O=C1COC2(CCN(S(=O)(=O)c3cnc(-c4ccc5cccnc5c4)s3)CC2)CN1C1CC1] |
| 1356 | 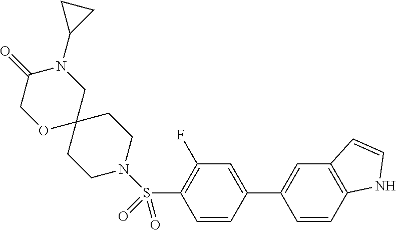 [SMILES: O=C1COC2(CCN(S(=O)(=O)c3ccc(-c4ccc5[nH]ccc5c4)cc3F)CC2)CN1C1CC1] |
| 1357 | 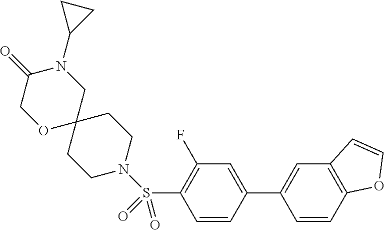 [SMILES: O=C1COC2(CCN(S(=O)(=O)c3ccc(-c4ccc5occc5c4)cc3F)CC2)CN1C1CC1] |
| 1358 | 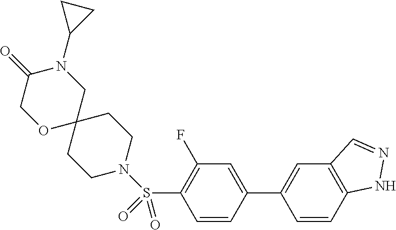 [SMILES: O=C1COC2(CCN(S(=O)(=O)c3ccc(-c4ccc5[nH]ncc5c4)cc3F)CC2)CN1C1CC1] |
| 1359 | 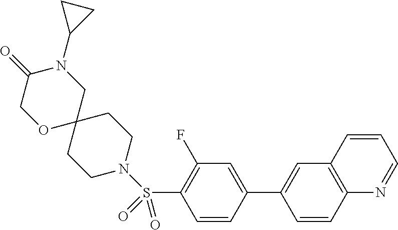 [SMILES: O=C1COC2(CCN(S(=O)(=O)c3ccc(-c4ccc5ncccc5c4)cc3F)CC2)CN1C1CC1] |
| 1360 | 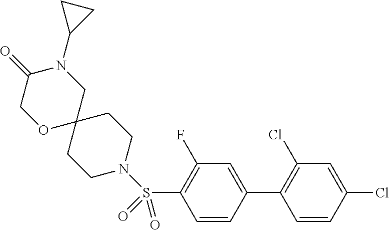 [SMILES: O=C1COC2(CCN(S(=O)(=O)c3ccc(-c4ccc(Cl)cc4Cl)cc3F)CC2)CN1C1CC1] |
| 1361 | 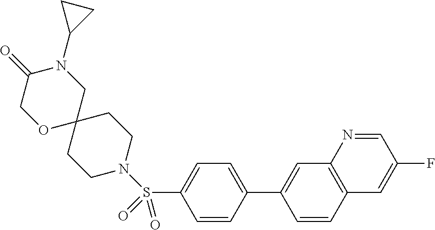 [SMILES: O=C1COC2(CCN(S(=O)(=O)c3ccc(-c4ccc5cc(F)cnc5c4)cc3)CC2)CN1C1CC1] |
| 1362 | 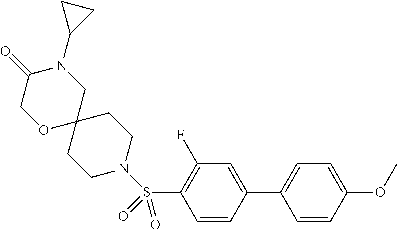 [SMILES: COc1ccc(-c2ccc(S(=O)(=O)N3CCC4(CC3)CN(C3CC3)C(=O)CO4)c(F)c2)cc1] |
| 1363 | 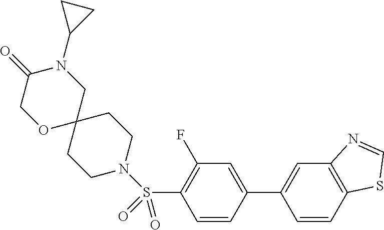 [SMILES: O=C1COC2(CCN(S(=O)(=O)c3ccc(-c4ccc5scnc5c4)cc3F)CC2)CN1C1CC1] |
| 1364 | 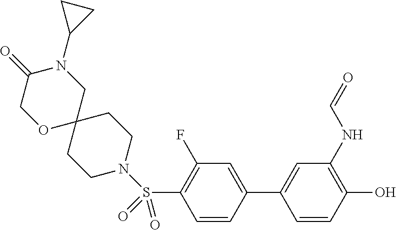 [SMILES: O=CNc1cc(-c2ccc(S(=O)(=O)N3CCC4(CC3)CN(C3CC3)C(=O)CO4)c(F)c2)ccc1O] |
| 1365 | 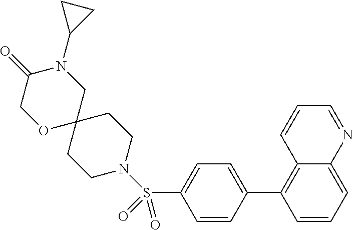 [SMILES: O=C1COC2(CCN(S(=O)(=O)c3ccc(-c4cccc5ncccc45)cc3)CC2)CN1C1CC1] |
| 1366 | 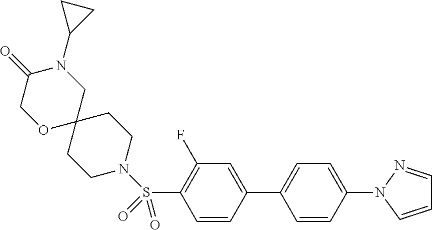 [SMILES: O=C1COC2(CCN(S(=O)(=O)c3ccc(-c4ccc(-n5cccn5)cc4)cc3F)CC2)CN1C1CC1] |
| 1367 | 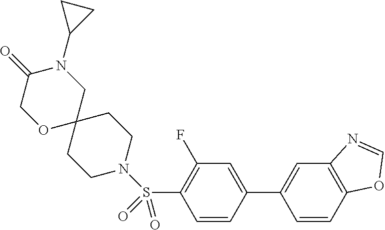 [SMILES: O=C1COC2(CCN(S(=O)(=O)c3ccc(-c4ccc5ocnc5c4)cc3F)CC2)CN1C1CC1] |
| 1368 | 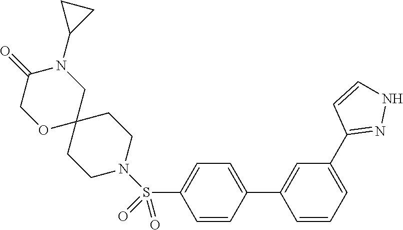 [SMILES: O=C1COC2(CCN(S(=O)(=O)c3ccc(-c4cccc(-c5cc[nH]n5)c4)cc3)CC2)CN1C1CC1] |
| 1369 | 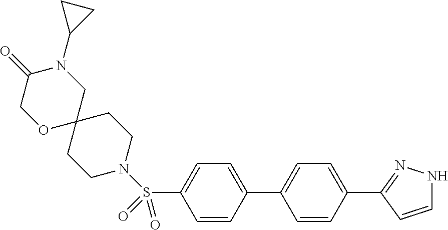 [SMILES: O=C1COC2(CCN(S(=O)(=O)c3ccc(-c4ccc(-c5cc[nH]n5)cc4)cc3)CC2)CN1C1CC1] |
| 1370 | 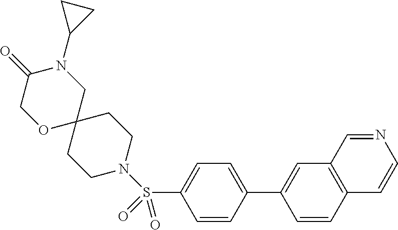 [SMILES: O=C1COC2(CCN(S(=O)(=O)c3ccc(-c4ccc5ccncc5c4)cc3)CC2)CN1C1CC1] |
| 1371 | 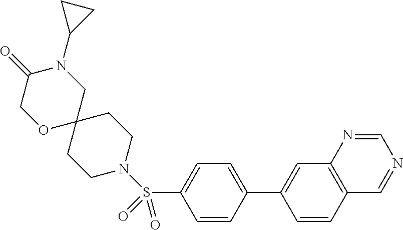 [SMILES: O=C1COC2(CCN(S(=O)(=O)c3ccc(-c4ccc5cncnc5c4)cc3)CC2)CN1C1CC1] |
| 1372 | 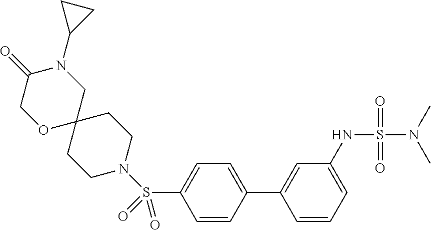 [SMILES: CN(C)S(=O)(=O)Nc1cccc(-c2ccc(S(=O)(=O)N3CCC4(CC3)CN(C3CC3)C(=O)CO4)cc2)c1] |
| 1373 | 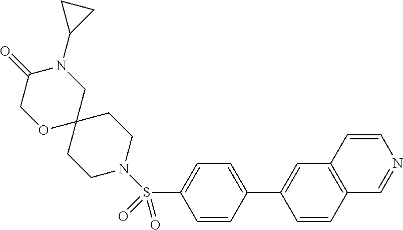 [SMILES: O=C1COC2(CCN(S(=O)(=O)c3ccc(-c4ccc5cnccc5c4)cc3)CC2)CN1C1CC1] |
| 1374 | 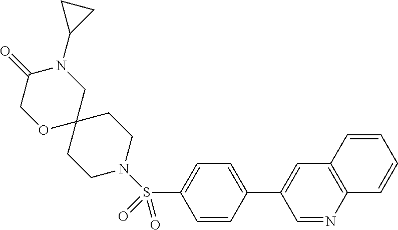 [SMILES: O=C1COC2(CCN(S(=O)(=O)c3ccc(-c4cnc5ccccc5c4)cc3)CC2)CN1C1CC1] |
| 1375 | 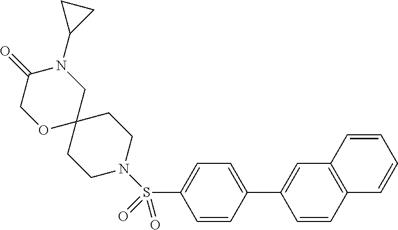 [SMILES: O=C1COC2(CCN(S(=O)(=O)c3ccc(-c4ccc5ccccc5c4)cc3)CC2)CN1C1CC1] |
| 1376 | 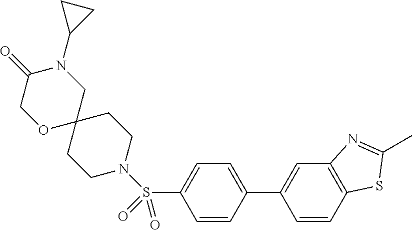 [SMILES: Cc1nc2cc(-c3ccc(S(=O)(=O)N4CCC5(CC4)CN(C4CC4)C(=O)CO5)cc3)ccc2s1] |
| 1377 | 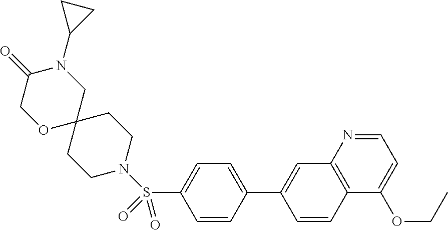 [SMILES: CCOc1ccnc2cc(-c3ccc(S(=O)(=O)N4CCC5(CC4)CN(C4CC4)C(=O)CO5)cc3)ccc12] |
| 1378 | 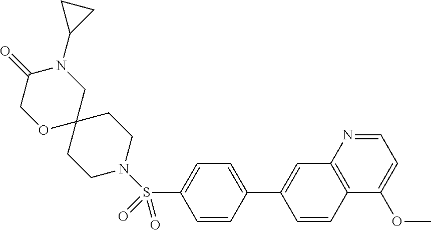 [SMILES: COc1ccnc2cc(-c3ccc(S(=O)(=O)N4CCC5(CC4)CN(C4CC4)C(=O)CO5)cc3)ccc12] |
| 1379 | 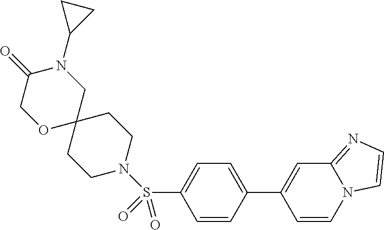 [SMILES: O=C1COC2(CCN(S(=O)(=O)c3ccc(-c4ccn5ccnc5c4)cc3)CC2)CN1C1CC1] |
| 1380 | 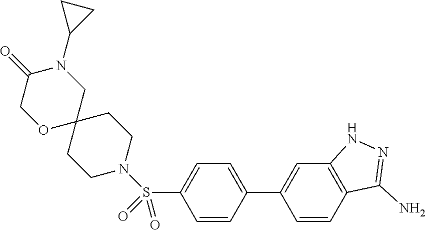 [SMILES: Nc1n[nH]c2cc(-c3ccc(S(=O)(=O)N4CCC5(CC4)CN(C4CC4)C(=O)CO5)cc3)ccc12] |
| 1381 | 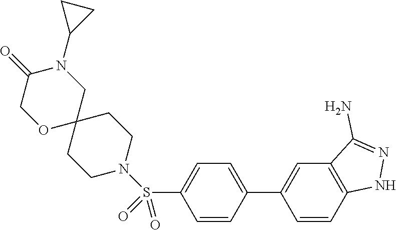 [SMILES: Nc1n[nH]c2ccc(-c3ccc(S(=O)(=O)N4CCC5(CC4)CN(C4CC4)C(=O)CO5)cc3)cc12] |
| 1382 | 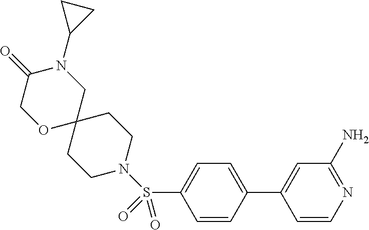 [SMILES: Nc1cc(-c2ccc(S(=O)(=O)N3CCC4(CC3)CN(C3CC3)C(=O)CO4)cc2)ccn1] |
| 1383 | 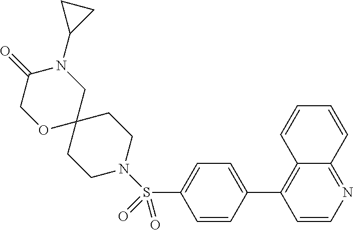 [SMILES: O=C1COC2(CCN(S(=O)(=O)c3ccc(-c4ccnc5ccccc45)cc3)CC2)CN1C1CC1] |
| 1384 | 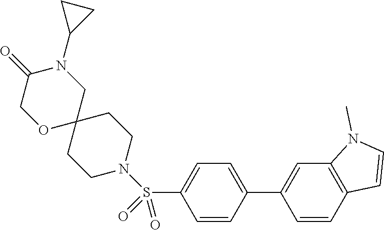 [SMILES: Cn1ccc2ccc(-c3ccc(S(=O)(=O)N4CCC5(CC4)CN(C4CC4)C(=O)CO5)cc3)cc21] |
| 1385 | 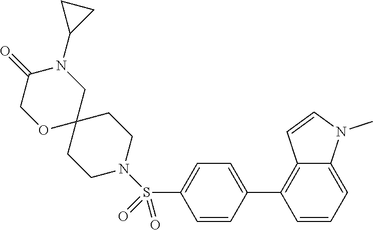 [SMILES: Cn1ccc2c(-c3ccc(S(=O)(=O)N4CCC5(CC4)CN(C4CC4)C(=O)CO5)cc3)cccc21] |
| 1386 | 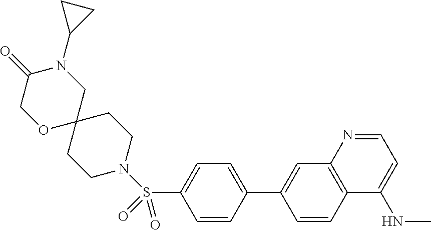 [SMILES: CNc1ccnc2cc(-c3ccc(S(=O)(=O)N4CCC5(CC4)CN(C4CC4)C(=O)CO5)cc3)ccc12] |
| 1387 | 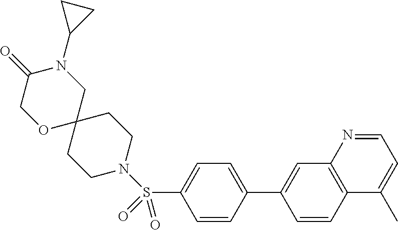 [SMILES: Cc1ccnc2cc(-c3ccc(S(=O)(=O)N4CCC5(CC4)CN(C4CC4)C(=O)CO5)cc3)ccc12] |
| 1388 | 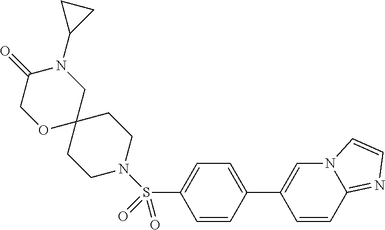 [SMILES: O=C1COC2(CCN(S(=O)(=O)c3ccc(-c4ccc5nccn5c4)cc3)CC2)CN1C1CC1] |
| 1389 | 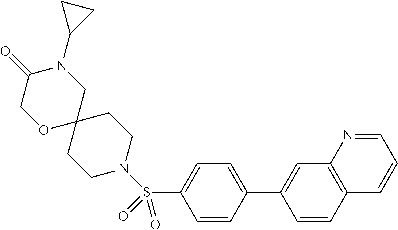 [SMILES: O=C1COC2(CCN(S(=O)(=O)c3ccc(-c4ccc5cccnc5c4)cc3)CC2)CN1C1CC1] |
| 1390 | 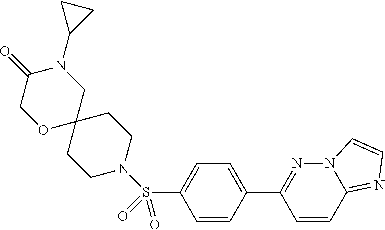 [SMILES: O=C1COC2(CCN(S(=O)(=O)c3ccc(-c4ccc5nccn5n4)cc3)CC2)CN1C1CC1] |
| 1391 | 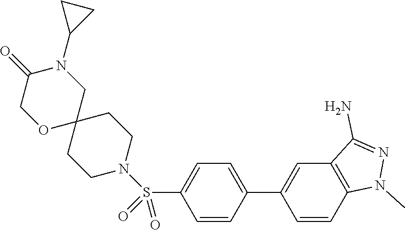 [SMILES: Cn1nc(N)c2cc(-c3ccc(S(=O)(=O)N4CCC5(CC4)CN(C4CC4)C(=O)CO5)cc3)ccc21] |
| 1392 | 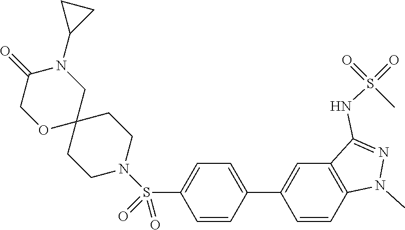 [SMILES: Cn1nc(NS(C)(=O)=O)c2cc(-c3ccc(S(=O)(=O)N4CCC5(CC4)CN(C4CC4)C(=O)CO5)cc3)ccc21] |
| 1393 | 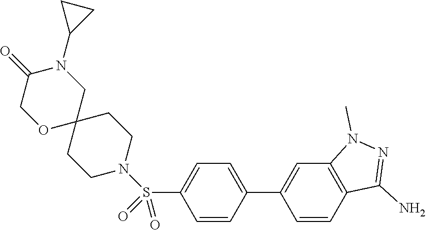 [SMILES: Cn1nc(N)c2ccc(-c3ccc(S(=O)(=O)N4CCC5(CC4)CN(C4CC4)C(=O)CO5)cc3)cc21] |
| 1394 | 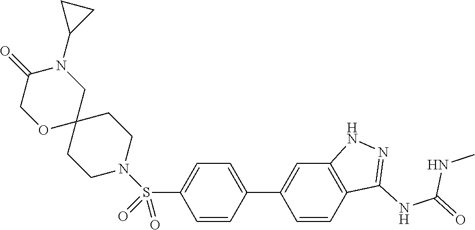 [SMILES: CNC(=O)Nc1n[nH]c2cc(-c3ccc(S(=O)(=O)N4CCC5(CC4)CN(C4CC4)C(=O)CO5)cc3)ccc12] |
| 1395 | 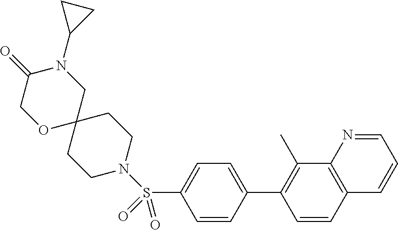 [SMILES: Cc1c(-c2ccc(S(=O)(=O)N3CCC4(CC3)CN(C3CC3)C(=O)CO4)cc2)ccc2cccnc12] |
| 1396 | 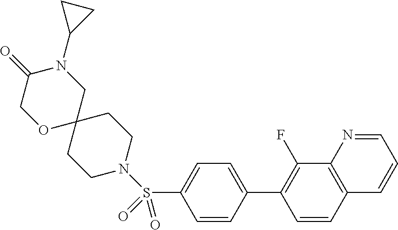 [SMILES: O=C1COC2(CCN(S(=O)(=O)c3ccc(-c4ccc5cccnc5c4F)cc3)CC2)CN1C1CC1] |
| 1397 | 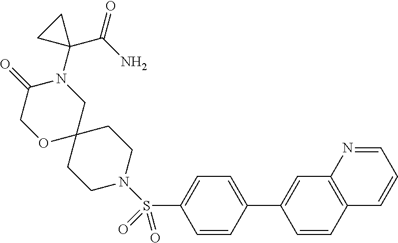 [SMILES: NC(=O)C1(N2CC3(CCN(S(=O)(=O)c4ccc(-c5ccc6cccnc6c5)cc4)CC3)OCC2=O)CC1] |
| 1398 | 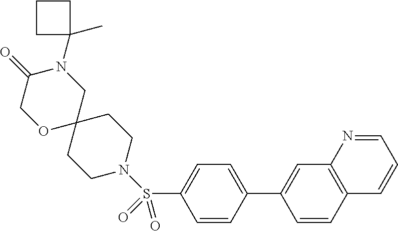 [SMILES: CC1(N2CC3(CCN(S(=O)(=O)c4ccc(-c5ccc6cccnc6c5)cc4)CC3)OCC2=O)CCC1] |
| 1399 | 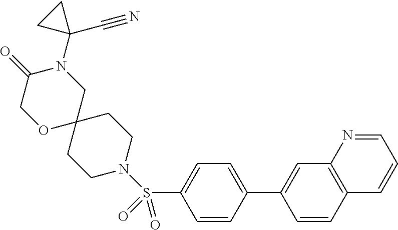 [SMILES: N#CC1(N2CC3(CCN(S(=O)(=O)c4ccc(-c5ccc6cccnc6c5)cc4)CC3)OCC2=O)CC1] |
| 1400 | 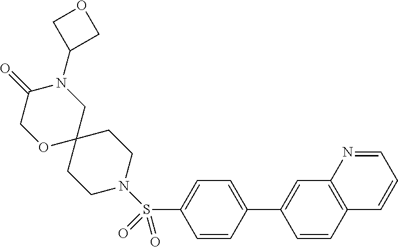 [SMILES: O=C1COC2(CCN(S(=O)(=O)c3ccc(-c4ccc5cccnc5c4)cc3)CC2)CN1C1COC1] |
| 1401 | 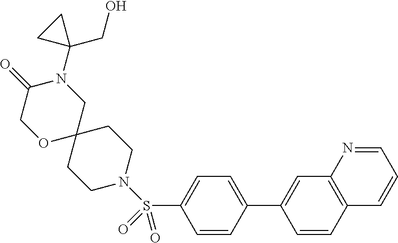 [SMILES: O=C1COC2(CCN(S(=O)(=O)c3ccc(-c4ccc5cccnc5c4)cc3)CC2)CN1C1(CO)CC1] |
| 1402 | 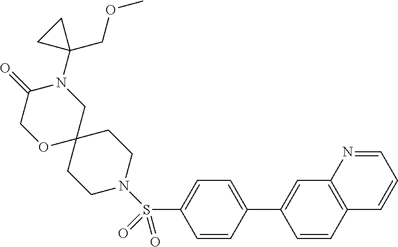 [SMILES: COCC1(N2CC3(CCN(S(=O)(=O)c4ccc(-c5ccc6cccnc6c5)cc4)CC3)OCC2=O)CC1] |
| 1403 | 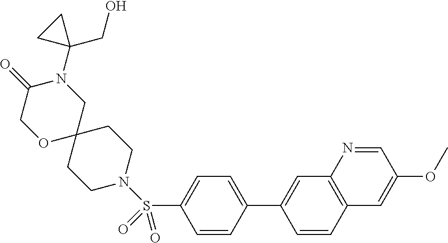 [SMILES: COc1cnc2cc(-c3ccc(S(=O)(=O)N4CCC5(CC4)CN(C4(CO)CC4)C(=O)CO5)cc3)ccc2c1] |
| 1404 | 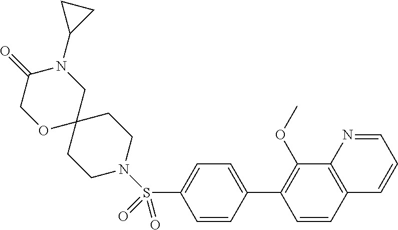 [SMILES: COc1c(-c2ccc(S(=O)(=O)N3CCC4(CC3)CN(C3CC3)C(=O)CO4)cc2)ccc2cccnc12] |
| 1405 | 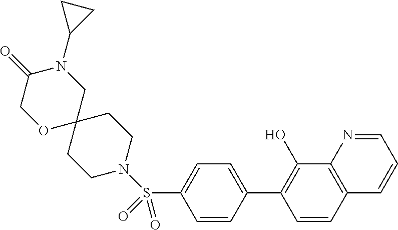 [SMILES: O=C1COC2(CCN(S(=O)(=O)c3ccc(-c4ccc5cccnc5c4O)cc3)CC2)CN1C1CC1] |
| 1406 | 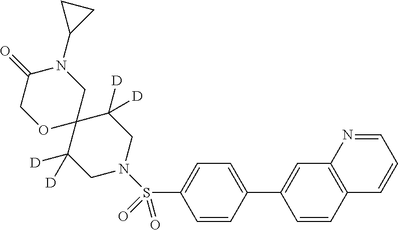 [SMILES: [2H]C1([2H])CN(S(=O)(=O)c2ccc(-c3ccc4cccnc4c3)cc2)CC([2H])([2H])C12CN(C1CC1)C(=O)CO2] |
| 1407 | 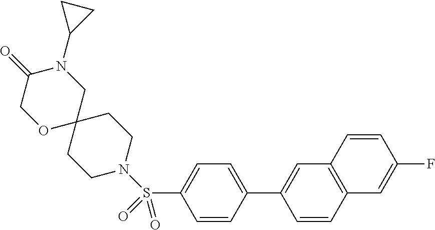 [SMILES: O=C1COC2(CCN(S(=O)(=O)c3ccc(-c4ccc5cc(F)ccc5c4)cc3)CC2)CN1C1CC1] |
| 1408 | 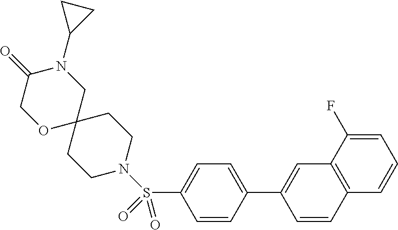 [SMILES: O=C1COC2(CCN(S(=O)(=O)c3ccc(-c4ccc5cccc(F)c5c4)cc3)CC2)CN1C1CC1] |
| 1409 | 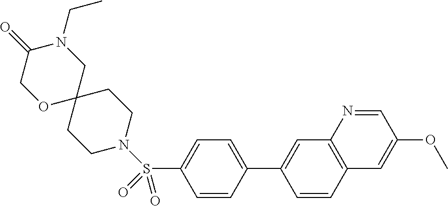 [SMILES: CCN1CC2(CCN(S(=O)(=O)c3ccc(-c4ccc5cc(OC)cnc5c4)cc3)CC2)OCC1=O] |
| 1410 | 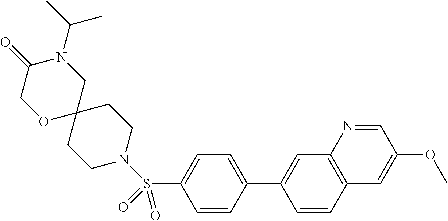 [SMILES: COc1cnc2cc(-c3ccc(S(=O)(=O)N4CCC5(CC4)CN(C(C)C)C(=O)CO5)cc3)ccc2c1] |
| 1411 | 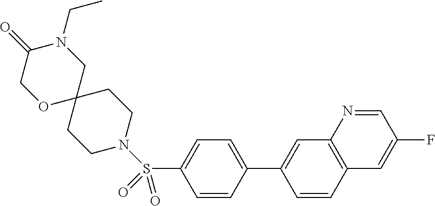 [SMILES: CCN1CC2(CCN(S(=O)(=O)c3ccc(-c4ccc5cc(F)cnc5c4)cc3)CC2)OCC1=O] |
| 1412 | 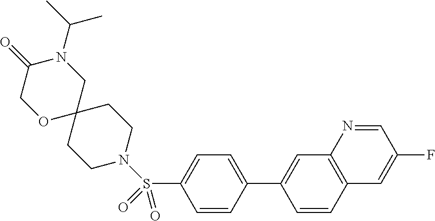 [SMILES: CC(C)N1CC2(CCN(S(=O)(=O)c3ccc(-c4ccc5cc(F)cnc5c4)cc3)CC2)OCC1=O] |
| 1413 | 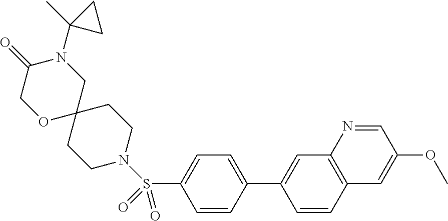 [SMILES: COc1cnc2cc(-c3ccc(S(=O)(=O)N4CCC5(CC4)CN(C4(C)CC4)C(=O)CO5)cc3)ccc2c1] |
| 1414 | 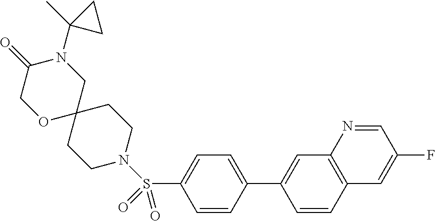 [SMILES: CC1(N2CC3(CCN(S(=O)(=O)c4ccc(-c5ccc6cc(F)cnc6c5)cc4)CC3)OCC2=O)CC1] |
| 1415 | 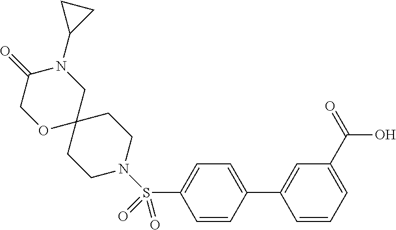 [SMILES: O=C(O)c1cccc(-c2ccc(S(=O)(=O)N3CCC4(CC3)CN(C3CC3)C(=O)CO4)cc2)c1] |
| 1416 | 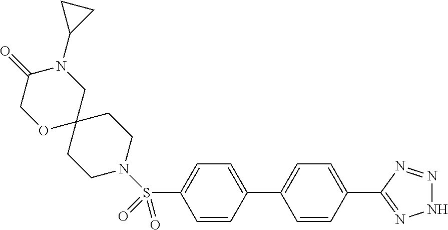 [SMILES: O=C1COC2(CCN(S(=O)(=O)c3ccc(-c4ccc(-c5nn[nH]n5)cc4)cc3)CC2)CN1C1CC1] |
| 1417 | 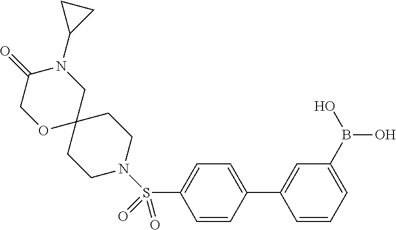 [SMILES: O=C1COC2(CCN(S(=O)(=O)c3ccc(-c4cccc(B(O)O)c4)cc3)CC2)CN1C1CC1] |
| 1418 | 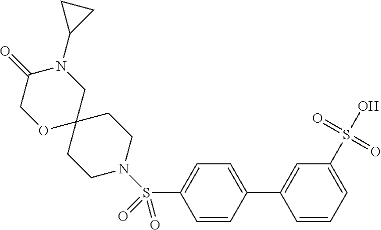 [SMILES: O=C1COC2(CCN(S(=O)(=O)c3ccc(-c4cccc(S(=O)(=O)O)c4)cc3)CC2)CN1C1CC1] |
| 1419 | 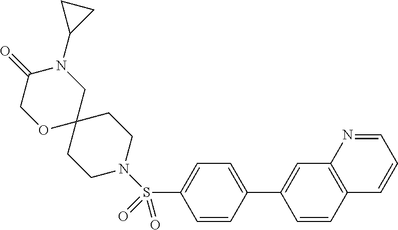 [SMILES: O=C1COC2(CCN(S(=O)(=O)c3ccc(-c4ccc5cccnc5c4)cc3)CC2)CN1C1CC1] |
| 1420 | 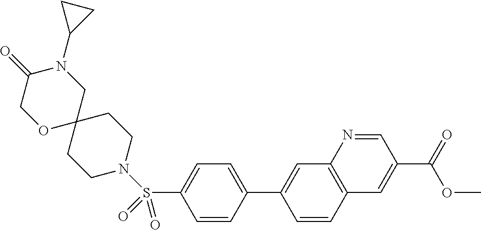 [SMILES: COC(=O)c1cnc2cc(-c3ccc(S(=O)(=O)N4CCC5(CC4)CN(C4CC4)C(=O)CO5)cc3)ccc2c1] |
| 1421 | 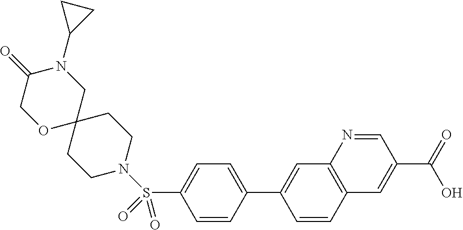 [SMILES: O=C(O)c1cnc2cc(-c3ccc(S(=O)(=O)N4CCC5(CC4)CN(C4CC4)C(=O)CO5)cc3)ccc2c1] |
| 1422 | 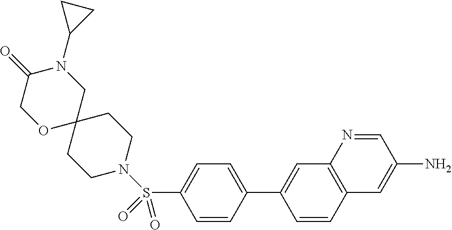 [SMILES: Nc1cnc2cc(-c3ccc(S(=O)(=O)N4CCC5(CC4)CN(C4CC4)C(=O)CO5)cc3)ccc2c1] |
| 1423 | 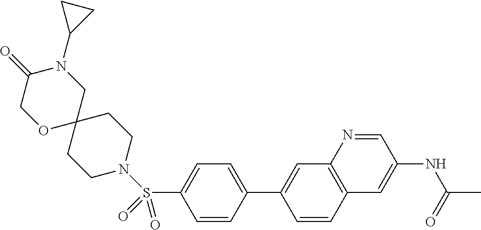 [SMILES: CC(=O)Nc1cnc2cc(-c3ccc(S(=O)(=O)N4CCC5(CC4)CN(C4CC4)C(=O)CO5)cc3)ccc2c1] |
| 1424 | 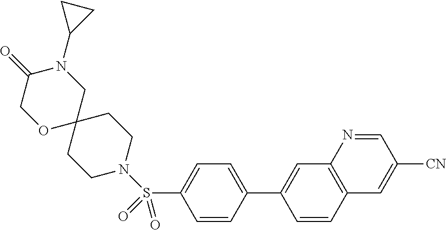 [SMILES: N#Cc1cnc2cc(-c3ccc(S(=O)(=O)N4CCC5(CC4)CN(C4CC4)C(=O)CO5)cc3)ccc2c1] |
| 1425 | 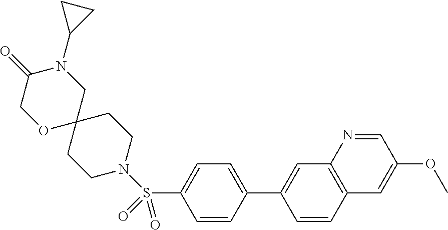 [SMILES: COc1cnc2cc(-c3ccc(S(=O)(=O)N4CCC5(CC4)CN(C4CC4)C(=O)CO5)cc3)ccc2c1] |
| 1426 | 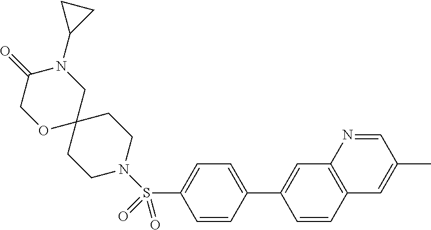 [SMILES: Cc1cnc2cc(-c3ccc(S(=O)(=O)N4CCC5(CC4)CN(C4CC4)C(=O)CO5)cc3)ccc2c1] |
| 1427 | 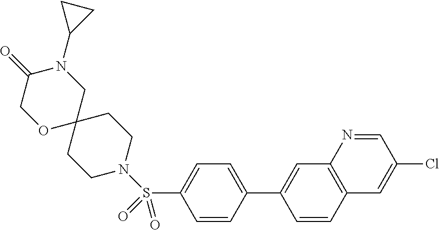 [SMILES: O=C1COC2(CCN(S(=O)(=O)c3ccc(-c4ccc5cc(Cl)cnc5c4)cc3)CC2)CN1C1CC1] |
| 1428 | 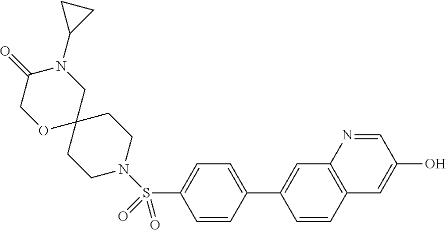 [SMILES: O=C1COC2(CCN(S(=O)(=O)c3ccc(-c4ccc5cc(O)cnc5c4)cc3)CC2)CN1C1CC1] |
| 1429 | 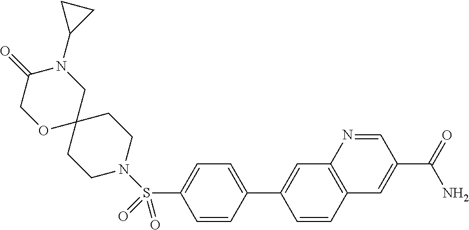 [SMILES: NC(=O)c1cnc2cc(-c3ccc(S(=O)(=O)N4CCC5(CC4)CN(C4CC4)C(=O)CO5)cc3)ccc2c1] |
| 1430 | 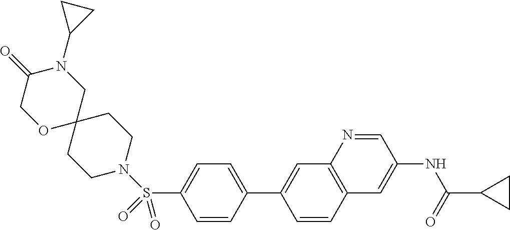 [SMILES: O=C(Nc1cnc2cc(-c3ccc(S(=O)(=O)N4CCC5(CC4)CN(C4CC4)C(=O)CO5)cc3)ccc2c1)C1CC1] |
| 1431 | 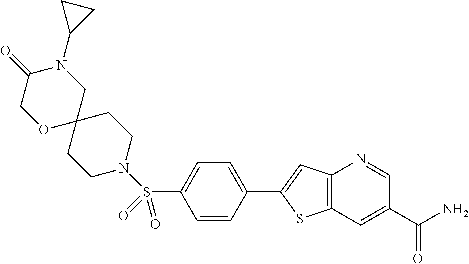 [SMILES: NC(=O)c1cnc2cc(-c3ccc(S(=O)(=O)N4CCC5(CC4)CN(C4CC4)C(=O)CO5)cc3)sc2c1] |
| 1432 | 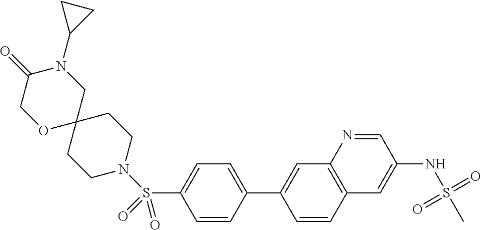 [SMILES: CS(=O)(=O)Nc1cnc2cc(-c3ccc(S(=O)(=O)N4CCC5(CC4)CN(C4CC4)C(=O)CO5)cc3)ccc2c1] |

In some embodiments, the compound has the structure of Formula (XXVI):

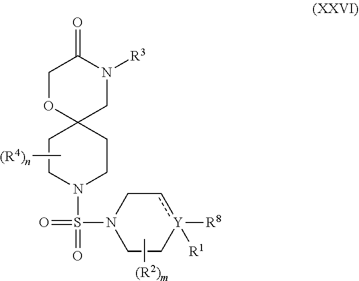

wherein R¹ is phenyl, 5- or 6-membered heteroaryl, napthyl, or 9- or 10-membered heterocyclyl wherein said phenyl, 5- or 6-membered heteroaryl, napthyl, or 9- or 10-membered heterocyclyl is optionally substituted with from 1 to 3 substituents independently selected from the group consisting of: C₁-C₆alkyl, —CF₃, C₃-C₇ cycloalkyl, —C(═O)C₁-C₄alkyl, —C₁-C₆alkylC₃-C₇cycloalkyl, —C(═O)C₃-C₇cycloalkyl, C(═O)(phenyl), —C(═O)OC₁-C₄alkyl, —C(═O)OH, —C(═O)NR⁵R⁶, —O(C₂-C₄alkyl)NR⁵R⁶, phenyl, —SO₂C₁-C₄alkyl, —SO₂NR⁵R⁶, cyano, oxo, hydroxyl, halogen, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁷C(═O)C₁-C₄alkyl, —NR⁷C(═O)NR⁵R⁶, —NR⁷SO₂C₁-C₄alkyl, —NR⁷SO₂NR⁵R⁶ and R⁹; each R² is independently selected from the group of C₁-C₆alkyl, cyano, C₁-C₆alkoxy, hydroxyl, and halogen; R³ is selected from the group consisting of: C₁-C₆alkyl, C₃-C₇cycloalkyl, hydroxyC₁-C₆alkyl-, and C₄-C₆heterocycloalkyl, wherein said C₁-C₆alkyl, C₃-C₇cycloalkyl, hydroxyC₁-C₆alkyl-, and C₄-C₆ heterocycloalkyl is optionally substituted with from 1 to 4 substituents independently selected from the group consisting of: halogen, C₁-C₆alkyl, —CF₃, C₃-C₇cycloalkyl, —C(═O)C₁-C₄alkyl, —C₁-C₆alkylC₃-C₇cycloalkyl, —C(═O)C₃-C₇cycloalkyl, —C(═O)(phenyl), —C(═O)OH, —C(═O)OC C₄alkyl, —C(═O)NR⁵R⁶, phenyl, —SO₂C C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁷C(O)C₁-C₄alkyl, —NR⁷CONR⁵R⁶, —NR⁷SO₂C₁-C₄alkyl, and —NR⁷SO₂NR⁵R⁶, and R⁹; each R⁴ is independently selected from the group consisting of halogen, hydroxyl, hydrogen, C₁-C₆alkoxy, and C₁-C₆alkyl; R⁵ is selected from the group consisting of hydrogen, C₁-C₄alkyl, phenyl, C₃-C₇cycloalkyl, —C₃-C₇alkylC₃-C₇cycloalkyl, and C₁-C₃alkyl-phenyl; R⁶ is hydrogen, C₁-C₄alkyl, C₃-C₇cycloalkyl, or —C₁-C₃alkylC₃-C₇cycloalkyl; or R⁵ and R⁶ taken together with the nitrogen to which they are attached represent a 4- to 7-membered saturated or unsaturated ring optionally containing one other heteroatom which is oxygen, nitrogen, or sulfur, wherein said ring is optionally substituted by 1 to 3 substituents independently selected from hydroxyl, C₁-C₃alkyl, and hydroxyC₁-C₄alkyl-; R⁷ is hydrogen or methyl; R⁸ is hydrogen, hydroxyl, or —OC₁-C₃alkyl; R⁹ is a 5- or 6-membered heteroaryl ring containing 1 to 4 heteroatoms selected from oxygen, nitrogen, and sulfur, which is optionally substituted with 1 or 2 substituents independently selected from halogen, C₁-C₄alkyl, —CF₃, C₁-C₄alkoxy, and —NR⁵R⁶; Y is C or N; when Y is N, R⁸ is absent; m is 0, 1, 2, 3, or 4; and n is 0, 1, 2, 3, or 4; or a pharmaceutically acceptable salt thereof.

In some embodiments, the compound has the structure of Formula (XXVI-A), (XXVI-B), or (XXVI-C):

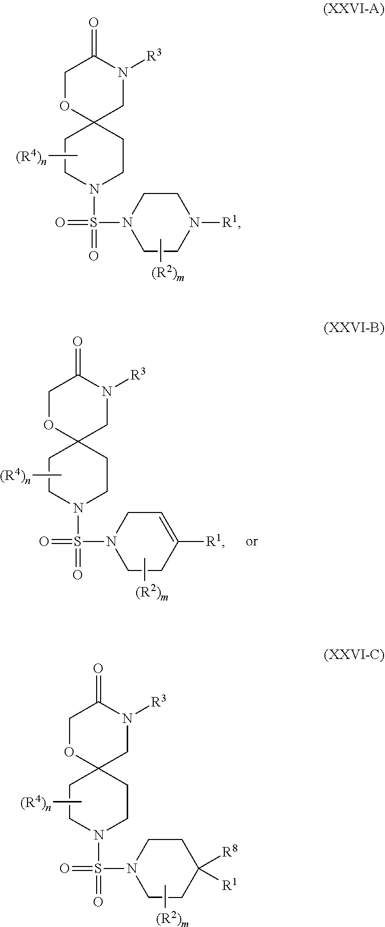

or a pharmaceutically acceptable salt thereof.

In some embodiments, R¹ is phenyl, 5- or 6-membered heteroaryl, napthyl, or 9- or 10-membered heterocyclyl wherein said phenyl, 5- or 6-membered heteroaryl, napthyl, or 9- or 10-membered heterocyclyl is optionally substituted with from 1 to 3 substituents independently selected from the group consisting of: C₁-C₆alkyl, —CF₃, C₃-Cycloalkyl, —C(═O)C₁-C₄alkyl, —C₁-C₆alkylC₃-C₇cycloalkyl, —C(═O)C₃-C₇cycloalkyl, —C(═O)(phenyl), —C(═O)OC₁-C₄alkyl, —C(═O)OH, —C(═O)NR⁵R⁶, —O(C₂-C₄alkyl)NR⁵R⁶, phenyl, —SO₂C₁-C₄alkyl, —SO₂NR⁵R⁶, cyano, oxo, hydroxyl, halogen, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁷C(═O)C₁-C₄alkyl, —NR⁷C(═O)NR⁵R⁶, —NR⁷SO₂C₁-C₄alkyl, —NR⁷SO₂NR⁵R⁶ and R⁹. In some embodiments, R¹ is benzothiazolyl, quinazolinyl, quinoxalinyl, cinnolinyl, indoyl, benzofuranyl, benzoxazoyl, indazoyl, benzimidazoyl, benzothienyl, phenyl, naphthyl, isoquinolinyl, or quinolinyl, wherein said benzothiazolyl, quinazolinyl, quinoxalinyl, cinnolinyl, indoyl, benzofuranyl, benzoxazoyl, indazoyl, benzimidazoyl, benzothienyl, phenyl, naphthyl, isoquinolinyl, or quinolinyl is optionally substituted with from 1 to 3 substituents independently selected from the group consisting of: C₁-C₆alkyl, —CF₃, C₃-C₇cycloalkyl, C(═O)C₁-C₄alkyl, —C₁-C₆alkylC₃-C₇cycloalkyl, —C(═O)C₃-C₇cycloalkyl, —C(═O)(phenyl), —C(═O)OC₁-C₄alkyl, —C(═O)OH, —C(═O)NR⁵R⁶, —O(C₂-C₄alkyl)NR⁵R⁶, —NHC(═O)C₁-C₄alkyl, phenyl, cyano, oxo, hydroxyl, halogen, C₁-C₄alkoxy, C₃-C₇cycloalkylC₁-C₄alkoxy, hydroxyC₁-C₄alkyl-, C₁-C₄alkoxyC₁-C₄alkyl-, —OCF₃, NR⁵R⁶, R⁵R⁶NC₁-C₄alkyl-, —NR⁷C(═O)C₁-C₄alkyl, and —NR⁷C(═O)NR⁵R⁶. In some embodiments, R¹ is selected from the group consisting of phenyl, benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, 1-H-indazolyl, benzimidazolyl, dihydrobenzimidazolyl, benzoxazolyl, dihydrobenzoxazolyl, benzothiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzothiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl, wherein said phenyl, benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, 1-H-indazolyl, benzimidazolyl, dihydrobenzimidazolyl, benzoxazolyl, dihydrobenzoxazolyl, benzothiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzothiadiazolyl, benzotriazolyl, triazolopyridinyl, purinyl, quinolinyl, tetrahydroquinolinyl, isoquinolinyl, tetrahydroisoquinolinyl, quinoxalinyl, Cinnolinyl, phthalazinyl, quinazolinyl, 1,5-naphthyridinyl, 1,6-naphthyridinyl, 1,7-naphthyridinyl, 1,8-naphthyridinyl, and pteridinyl, is optionally substituted 1 to 3 times independently with halogen, C₁-C₄alkyl, —CF₃, C₃-C₇cycloalkyl, C(═O)C₁-C₄alkyl, —C(═O)C₃-C₇cycloalkyl, —C(═O)phenyl, C(═O)OH, —C(═O)OC₁-C₄alkyl, —C(═O)NR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —O(C₂-C₄alkyl)NR⁵R⁶, —NR⁵R⁶, R⁵R⁶NC C₄alkyl-, —NR⁷C(O)C₁-C₄alkyl, —NR⁷C(O)NR⁵R⁶, —NR⁷SO₂C₁-C₄alkyl, —NR⁷SO₂NR⁵R⁶, or R⁹. In some embodiments, R¹ is benzothiazolyl, phenyl, naphthyl, isoquinolinyl, or quinolinyl, wherein said benzothiazolyl, phenyl, naphthyl, isoquinolinyl, or quinolinyl is optionally substituted with from 1 to 3 substituents independently selected from the group consisting of: C₁-C₆alkyl, —CF₃, C₃-C₇cycloalkyl, —C(═O)C₁-C₄alkyl, —C₁-C₆alkylC₃-C₇cycloalkyl, —C(═O)C₃-C₇cycloalkyl, —C(═O)(phenyl), —C(═O)OC₁-C₄alkyl, —C(═O)OH, —C(═O)NR⁵R⁶, —O(C₂-C₄alkyl)NR⁵R⁶, —NHC(═O)C C₄alkyl, phenyl, cyano, oxo, hydroxyl, halogen, C₁-C₄alkoxy, C₃-C₇cycloalkylC₁-C₄alkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁷C(═O)C₁-C₄alkyl, and —NR⁷C(═O)NR⁵R⁶. In some embodiments, R² is independently selected from the group of C₁-C₆alkyl, cyano, C₁-C₆alkoxy, hydroxyl, and halogen. In some embodiments, R³ is selected from the group consisting of: C₁-C₆alkyl, C₃-C₇cycloalkyl, hydroxyC₁-C₆alkyl-, and C₄-C₆ heterocycloalkyl, wherein said C₁-C₆alkyl, C₃-C₇cycloalkyl, hydroxyC₁-C₆alkyl-, and C₄-C₆heterocycloalkyl is optionally substituted with from 1 to 4 substituents independently selected from the group consisting of: halogen, C₁-C₆alkyl, —CF₃, C₃-C₇cycloalkyl, —C(═O)C₁-C₄alkyl, —C₁-C₆alkylC₃-C₇cycloalkyl, —C(═O)C₃-C₇cycloalkyl, —C(═O)(phenyl), —C(═O)OH, —C(═O)OC₁-C₄alkyl, —C(═O)NR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁷C(O)C₁-C₄alkyl, —NR⁷CONR⁵R⁶, —NR⁷SO₂C₁-C₄alkyl, and —NR⁷SO₂NR⁵R⁶, and R⁹. In some embodiments, R³ is C₁-C₆alkyl or C₃-C₆cycloalkyl wherein said C₁-C₆alkyl and C₃-C₆ cycloalkyl is optionally substituted by C₁-C₃alkyl. In some embodiments, R³ is C₁-C₆alkyl. In some embodiments, R³ is C₃-C₆cycloalkyl. In some embodiments, R³ is C₃-C₆cycloalkyl, wherein said C₃-C₆ cycloalkyl is optionally substituted by C₁-C₃alkyl. In some embodiments, R³ is cyclopropyl. In some embodiments, R⁴ is independently selected from the group consisting of halogen, hydroxyl, hydrogen, C₁-C₆alkoxy, and C₁-C₆alkyl. In some embodiments, R⁴ is halogen. In some embodiments, R⁵ is selected from the group consisting of hydrogen, C₁-C₄alkyl, phenyl, C₃-C₇cycloalkyl, C₃-C₇alkylC₃-C₇cycloalkyl, and C₁-C₃alkyl-phenyl. In some embodiments, R⁶ is hydrogen, C₁-C₄alkyl, C₃-C₇cycloalkyl, or —C₁-C₃alkylC₃-C₇cycloalkyl. In some embodiments, R⁵ and R⁶ taken together with the nitrogen to which they are attached represent a 4- to 7-membered saturated or unsaturated ring optionally containing one other heteroatom which is oxygen, nitrogen, or sulfur, wherein said ring is optionally substituted by 1 to 3 substituents independently selected from hydroxyl, C₁-C₃alkyl, and hydroxyC₁-C₄alkyl-. In some embodiments, R⁵ and R⁶ taken together with the nitrogen to which they are attached represent a 5- to 6-membered saturated or unsaturated ring optionally containing one other heteroatom which is oxygen, nitrogen, or sulfur, wherein said ring is optionally substituted by 1 to 3 substituents independently selected from hydroxyl, C₁-C₃alkyl, and hydroxyC₁-C₄alkyl-. In some embodiments, R⁷ is hydrogen or methyl. In some embodiments, R⁸ is hydrogen, hydroxyl, or —OC₁-C₃alkyl. In some embodiments, R⁹ is a 5- or 6-membered heteroaryl ring containing 1 to 4 heteroatoms selected from oxygen, nitrogen, and sulfur, which is optionally substituted with 1 or 2 substituents independently selected from halogen, C₁-C₄alkyl, —CF₃, C₁-C₄alkoxy, and —NR⁵R⁶. In some embodiments, R⁹ is a 5-membered heteroaryl ring containing 1 to 4 heteroatoms selected from oxygen, nitrogen, and sulfur, which is optionally substituted with 1 or 2 substituents independently selected from halogen, C₁-C₄alkyl, CF₃, C₁-C₄alkoxy, and —NR⁵R⁶. In some embodiments, R⁹ is furanyl, thienyl, pyrrolyl, imidazolyl, pyrazolyl, triazolyl, tetrazolyl, thiazolyl, oxazolyl, isoxazolyl, oxadiazolyl, thiadiazolyl, or isothiazolyl. In some embodiments, R⁹ is a 6-membered heteroaryl ring containing 1 to 4 heteroatoms selected from oxygen, nitrogen, and sulfur, which is optionally substituted with 1 or 2 substituents independently selected from halogen, C₁-C₄alkyl, —CF₃, C₁-C₄alkoxy, and —NR⁵R⁶. In some embodiments, R⁹ is pyridinyl, pyridazinyl, pyrazinyl, or pyrimidinyl. Suitably, Y is C, or N, and when Y is N, R⁸ is absent. In an embodiment of this invention, Y is C. In another embodiment of this invention, Y is N. Suitably, m is 0, 1, 2, 3, or 4. In an embodiment of this invention, m is 0 or 1. In another specific embodiment of this invention, m is 0. In another embodiment of this invention, m is 1. Suitably, n is 0, 1, 2, 3, or 4. In another embodiment of this invention, n is 0 or 1. In another embodiment of this invention, n is 0. In another embodiment of this invention, n is 1. In some embodiments, at least one of m or n is other than zero and there is an excess of one enantiomer over the other.

In some embodiments, the compound is 4-cyclopropyl-9-{[4-(7-quinolinyl)-1-piperazinyl]sulfonyl}-1-oxa-4,9-diazaspiro[5,5]undecan-3-one; 4-(1-methylcyclopropyl)-9-{[4-(7-quinolinyl)-1-piperazinyl]sulfonyl}-1-oxa-4,9-diazaspiro[5,5]undecan-3-one; 4-cyclopropyl-9-{[4-(7-quinolinyl)-3,6-dihydro-1 (2H)-pyridinyl]sulfonyl}-1-oxa-4,9-diazaspiro[5,5]undecan-3-one; 4-cyclopropyl-9-((4-(quinolin-7-yl)piperidin-1-yl)sulfonyl)-1-oxa-4,9-diazaspiro[5,5] undecan-3-one; 4-cyclopropyl-9-{[3-methyl-4-(7-quinolinyl)-1-piperaznyl]sulfonyl}-1-oxa-4,9-diazaspiro[5,5]undecan-3-one; (+)-4-cyclopropyl-9-((3-methyl-4-(quinolin-7-yl)piperazin-1-yl)sulfonyl)-1-oxa-4,9-diazaspiro[5,5]undecan-3-one; (−)-4-cyclopropyl-9-((2-methyl-4-(quinolin-7-yl)piperazin-1-yl)sulfonyl)-1-oxa-4,9-diazaspiro[5,5]undecan-3-one; 4-cyclopropyl-9-((2-methyl-4-(quinolin-7-yl)piperazin-1-yl)sulfonyl)-1-oxa-4,9-diazaspiro[5,5]undecan-3-one; 4-cyclopropyl-9-{[4-(6-isoquinolinyl)-1-piperaznyl]sulfonyl}-1-oxa-4,9-diazaspiro[5,5]undecan-3-one; 4-cyclopropyl-9-{[4-(2-naphthalenyl)-1-piperaznyl]sulfonyl}-1-oxa-4,9-diazaspiro[5.5]undecan-3-one; 4-cyclopropyl-9-({4-[4-(methyloxy)phenyl]-1-piperazinyl} sulfonyl)-1-oxa-4,9-diazaspiro[5,5]undecan-3-one; 4-cyclopropyl-9-{[4-(5-quinolinyl)-1-piperaznyl]sulfonyl}-1-oxa-4,9-diazaspiro[5,5]undecan-3-one; 6-(4-((4-cyclopropyl-3-oxo-1-oxa-4,9-diazaspiro[5,5]undecan-9-yl)sulfonyl)piperazin-1-yl)-2-naphthonitrile; 9-{[4-(1,3-benzothiazol-5-yl)-1-piperaznyl]sulfonyl}-4-cyclopropyl-1-oxa-4,9-diazaspiro[5,5]undecan-3-one; 4-{4-[(4-cyclopropyl-3-oxo-1-oxa-4,9-diazaspiro[5,5]undec-9-yl)sulfonyl]-1-piperazinyl}benzonitrile; 9-{[4-(4-chlorophenyl)-1-piperazinyl]sulfonyl}-4-cyclopropyl-1-oxa-4,9-diazaspiro[5,5]undecan-3-one; 7-(4-((4-cyclopropyl-3-oxo-1-oxa-4,9-diazaspiro[5,5]undecan-9-yl)sulfonyl)piperazin-1-yl)quinoline-3-carboxylate; 7-(4-((4-cyclopropyl-3-oxo-1-oxa-4,9-diazaspiro[5,5]undecan-9-yl)sulfonyl)piperazin-1-yl)quinoline-3-carboxamide; 7-(4-((4-cyclopropyl-3-oxo-1-oxa-4,9-diazaspiro[5,5]undecan-9-yl)sulfonyl)piperazin-1-yl)quinoline-3-carbonitrile; 4-cyclopropyl-9-((4-(3-methoxyquinolin-7-yl)piperazin-1-yl)sulfonyl)-1-oxa-4,9-diazaspiro[5,5]undecan-3-one; N-(7-(4-((4-cyclopropyl-3-oxo-1-oxa-4,9-diazaspiro[5,5]undecan-9-yl)sulfonyl)piperazin-1-yl)quinolin-3-yl)acetamide; 4-cyclopropyl-9-((4-(3-hydroxyquinolin-7-yl)piperazin-1-yl)sulfonyl)-1-oxa-4,9-diazaspiro[5,5]undecan-3-one; 9-((4-(3-chloroquinolin-7-yl)piperazin-1-yl)sulfonyl)-4-cyclopropyl-1-oxa-4,9-diazaspiro[5,5]undecan-3-one; 4-(1,1-dimethylpropyl)-9-{[4-(7-quinolinyl)-1-piperazinyl] sulfonyl}-1-oxa-4,9-diazaspiro[5,5]undecan-3-one; 4-cyclopropyl-9-((4-(6-fluoronaphthalen-2-yl)piperazin-1-yl)sulfonyl)-1-oxa-4,9-diazaspiro[5,5]undecan-3-one; 4-cyclopropyl-9-((4-(6-methylnaphthalen-2-yl)piperazin-1-yl)sulfonyl)-1-oxa-4,9-diazaspiro[5,5]undecan-3-one; 4-cyclopropyl-9-((4-(6-methoxynaphthalen-2-yl)piperazin-1-yl)sulfonyl)-1-oxa-4,9-diazaspiro[5,5]undecan-3-one; 4-cyclopropyl-9-((4-(8-fluoronaphthalen-2-yl)piperazin-1-yl)sulfonyl)-1-oxa-4,9-diazaspiro[5,5]undecan-3-one; 4-cyclopropyl-9-((4-(4-fluoronaphthalen-1-yl)piperazin-1-yl)sulfonyl)-1-oxa-4,9-diazaspiro[5,5]undecan-3-one; 4-cyclopropyl-9-((4-(6-hydroxynaphthalen-2-yl)piperazin-1-yl)sulfonyl)-1-oxa-4,9-diazaspiro[5,5]undecan-3-one; trans-4-cyclopropyl-7-fluoro-9-{[4-(7-quinolinyl)-1-piperazinyl]sulfonyl}-1-oxa-4,9-diazaspiro[5,5]undecan-3-one; (−)-trans-4-cyclopropyl-7-fluoro-9-{[4-(7-quinolinyl)-1-piperazinyl] sulfonyl}-1-oxa-4,9-diazaspiro[5,5]undecan-3-one; (+)-trans-4-cyclopropyl-7-fluoro-9-{[4-(7-quinolinyl)-1-piperazinyl]sulfonyl}-1-oxa-4,9-diazaspiro[5,5]undecan-3-one; or cis-4-cyclopropyl-7-fluoro-9-{[4-(7-quinolinyl)-1-piperazinyl]sulfonyl}-1-oxa-4,9-diazaspiro[5,5]undecan-3-one; or pharmaceutically acceptable salts thereof.

In some embodiments, the compound is one of the following:

Compound

| | |
|---|---|
| 1433 | 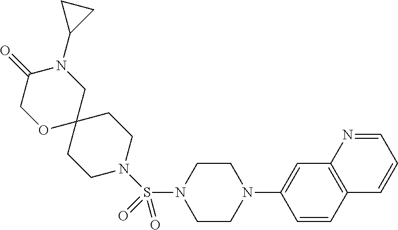 [SMILES: O=C1COC2(CCN(S(=O)(=O)N3CCN(c4ccc5cccnc5c4)CC3)CC2)CN1C1CC1] |
| 1434 | 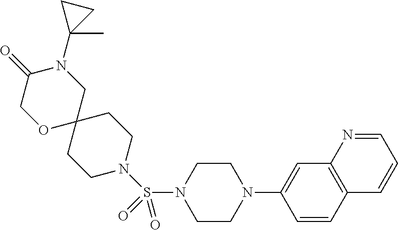 [SMILES: CC1(N2CC3(CCN(S(=O)(=O)N4CCN(c5ccc6cccnc6c5)CC4)CC3)OCC2=O)CC1] |
| 1435 | 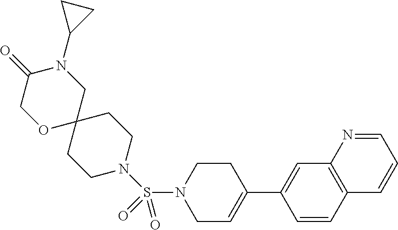 [SMILES: O=C1COC2(CCN(S(=O)(=O)N3CC=C(c4ccc5cccnc5c4)CC3)CC2)CN1C1CC1] |
| 1436 | 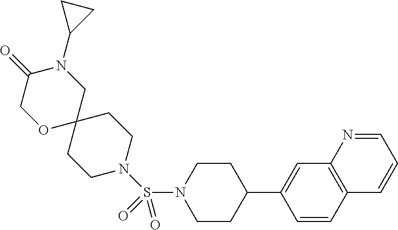 [SMILES: O=C1COC2(CCN(S(=O)(=O)N3CCC(c4ccc5cccnc5c4)CC3)CC2)CN1C1CC1] |
| 1437 | 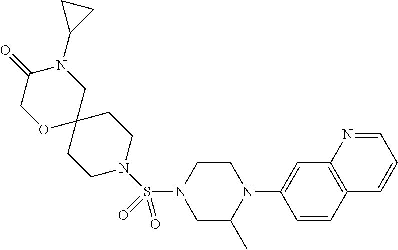 [SMILES: CC1CN(S(=O)(=O)N2CCC3(CC2)CN(C2CC2)C(=O)CO3)CCN1c1ccc2cccnc2c1] |
| 1438 | 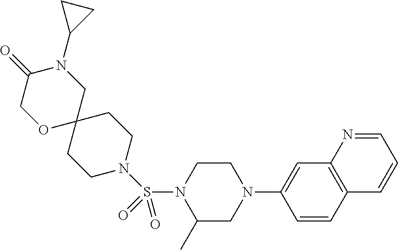 [SMILES: CC1CN(c2ccc3cccnc3c2)CCN1S(=O)(=O)N1CCC2(CC1)CN(C1CC1)C(=O)CO2] |
| 1439 | 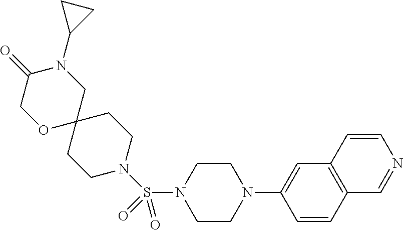 [SMILES: O=C1COC2(CCN(S(=O)(=O)N3CCN(c4ccc5cnccc5c4)CC3)CC2)CN1C1CC1] |
| 1440 | 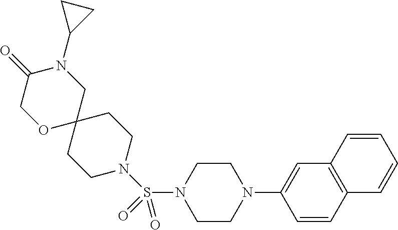 [SMILES: O=C1COC2(CCN(S(=O)(=O)N3CCN(c4ccc5ccccc5c4)CC3)CC2)CN1C1CC1] |
| 1441 | 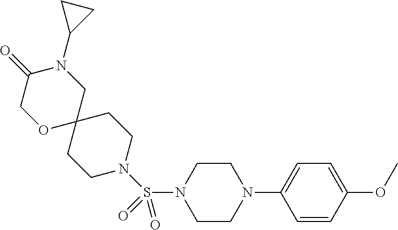 [SMILES: COc1ccc(N2CCN(S(=O)(=O)N3CCC4(CC3)CN(C3CC3)C(=O)CO4)CC2)cc1] |
| 1442 | 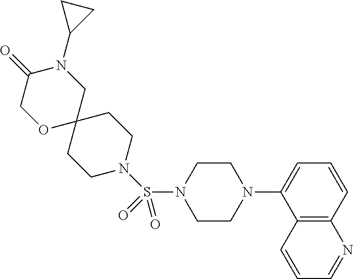 [SMILES: O=C1COC2(CCN(S(=O)(=O)N3CCN(c4cccc5ncccc45)CC3)CC2)CN1C1CC1] |
| 1443 | 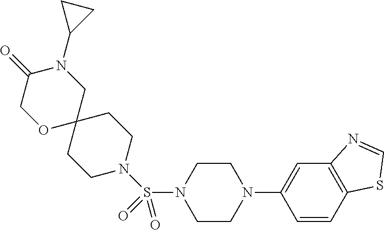 [SMILES: O=C1COC2(CCN(S(=O)(=O)N3CCN(c4ccc5scnc5c4)CC3)CC2)CN1C1CC1] |
| 1444 | 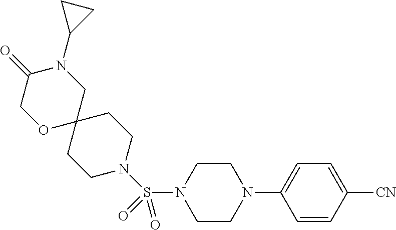 [SMILES: N#Cc1ccc(N2CCN(S(=O)(=O)N3CCC4(CC3)CN(C3CC3)C(=O)CO4)CC2)cc1] |
| 1445 | 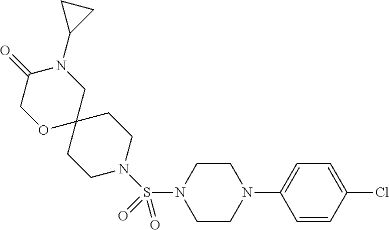 [SMILES: O=C1COC2(CCN(S(=O)(=O)N3CCN(c4ccc(Cl)cc4)CC3)CC2)CN1C1CC1] |
| 1446 | 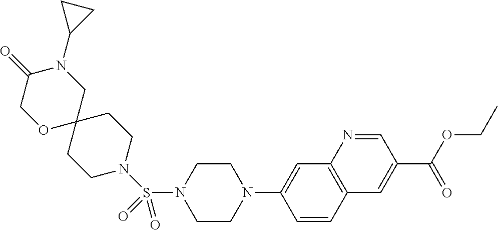 [SMILES: CCOC(=O)c1cnc2cc(N3CCN(S(=O)(=O)N4CCC5(CC4)CN(C4CC4)C(=O)CO5)CC3)ccc2c1] |
| 1447 | 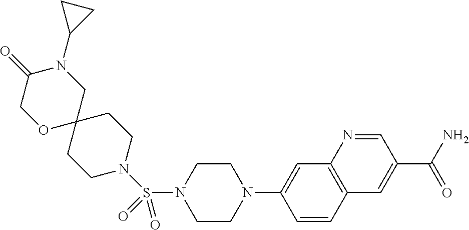 [SMILES: NC(=O)c1cnc2cc(N3CCN(S(=O)(=O)N4CCC5(CC4)CN(C4CC4)C(=O)CO5)CC3)ccc2c1] |
| 1448 | 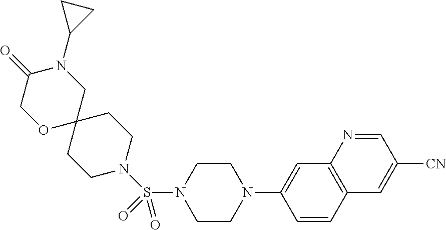 [SMILES: N#Cc1cnc2cc(N3CCN(S(=O)(=O)N4CCC5(CC4)CN(C4CC4)C(=O)CO5)CC3)ccc2c1] |
| 1449 | 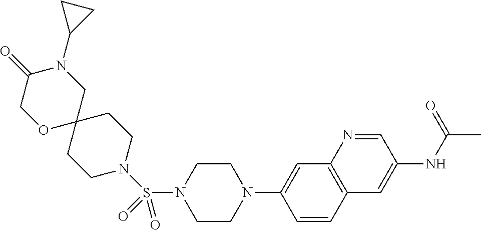 [SMILES: CC(=O)Nc1cnc2cc(N3CCN(S(=O)(=O)N4CCC5(CC4)CN(C4CC4)C(=O)CO5)CC3)ccc2c1] |
| 1450 | 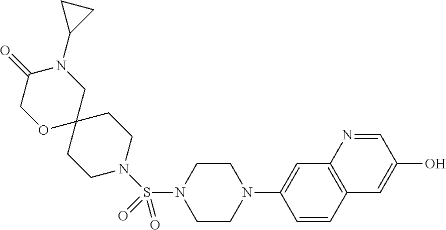 [SMILES: O=C1COC2(CCN(S(=O)(=O)N3CCN(c4ccc5cc(O)cnc5c4)CC3)CC2)CN1C1CC1] |
| 1451 | 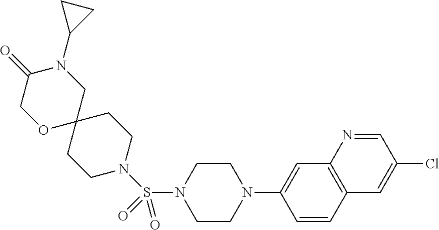 [SMILES: O=C1COC2(CCN(S(=O)(=O)N3CCN(c4ccc5cc(Cl)cnc5c4)CC3)CC2)CN1C1CC1] |
| 1452 | 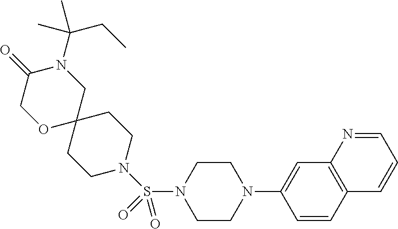 [SMILES: CCC(C)(C)N1CC2(CCN(S(=O)(=O)N3CCN(c4ccc5cccnc5c4)CC3)CC2)OCC1=O] |
| 1453 | 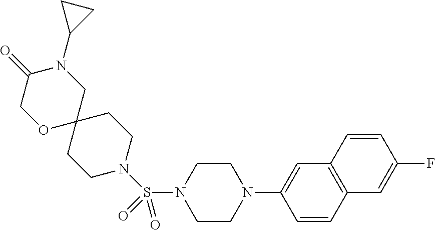 [SMILES: O=C1COC2(CCN(S(=O)(=O)N3CCN(c4ccc5cc(F)ccc5c4)CC3)CC2)CN1C1CC1] |
| 1454 | 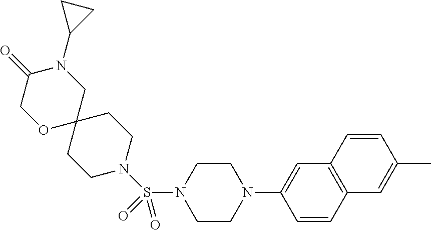 [SMILES: Cc1ccc2cc(N3CCN(S(=O)(=O)N4CCC5(CC4)CN(C4CC4)C(=O)CO5)CC3)ccc2c1] |
| 1455 | 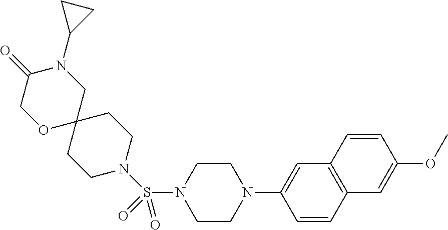 [SMILES: COc1ccc2cc(N3CCN(S(=O)(=O)N4CCC5(CC4)CN(C4CC4)C(=O)CO5)CC3)ccc2c1] |
| 1456 | 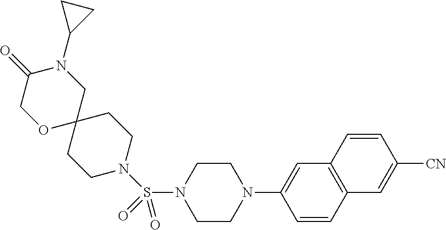 [SMILES: N#Cc1ccc2cc(N3CCN(S(=O)(=O)N4CCC5(CC4)CN(C4CC4)C(=O)CO5)CC3)ccc2c1] |
| 1457 | 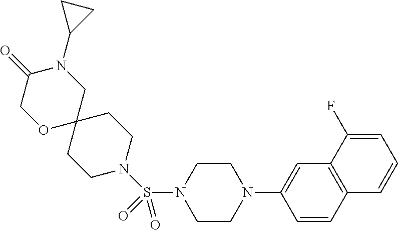 [SMILES: O=C1COC2(CCN(S(=O)(=O)N3CCN(c4ccc5cccc(F)c5c4)CC3)CC2)CN1C1CC1] |
| 1458 | 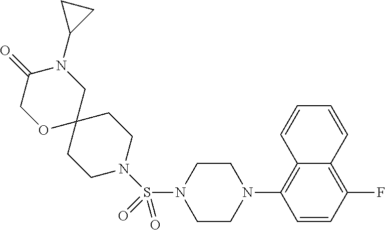 [SMILES: O=C1COC2(CCN(S(=O)(=O)N3CCN(c4ccc(F)c5ccccc45)CC3)CC2)CN1C1CC1] |
| 1459 | 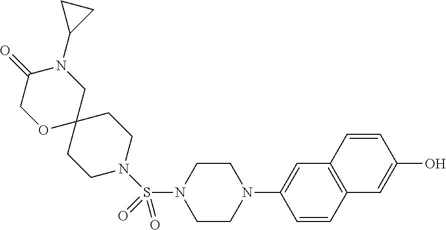 [SMILES: O=C1COC2(CCN(S(=O)(=O)N3CCN(c4ccc5cc(O)ccc5c4)CC3)CC2)CN1C1CC1] |
| 1460 | 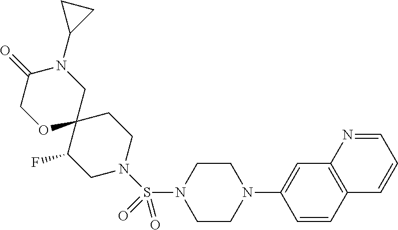 [SMILES: O=C1CO[C@]2(CCN(S(=O)(=O)N3CCN(c4ccc5cccnc5c4)CC3)C[C@@H]2F)CN1C1CC1] |
| 1461 | 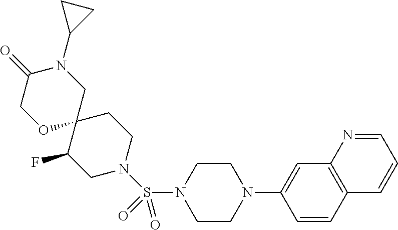 [SMILES: O=C1CO[C@@]2(CCN(S(=O)(=O)N3CCN(c4ccc5cccnc5c4)CC3)C[C@H]2F)CN1C1CC1] |
| 1462 | 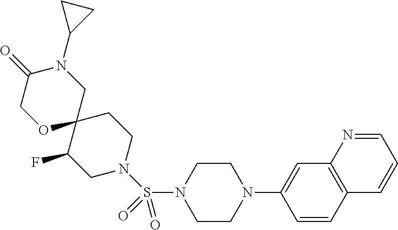 [SMILES: O=C1CO[C@]2(CCN(S(=O)(=O)N3CCN(c4ccc5cccnc5c4)CC3)C[C@H]2F)CN1C1CC1] |
| 1463 | 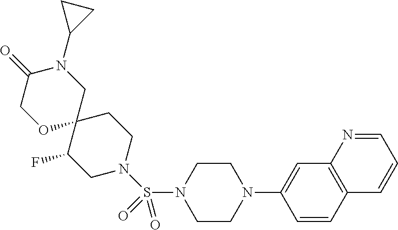 [SMILES: O=C1CO[C@@]2(CCN(S(=O)(=O)N3CCN(c4ccc5cccnc5c4)CC3)C[C@@H]2F)CN1C1CC1] |
| 1464 | 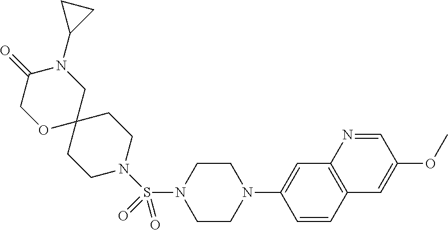 [SMILES: COc1cnc2cc(N3CCN(S(=O)(=O)N4CCC5(CC4)CN(C4CC4)C(=O)CO5)CC3)ccc2c1] |

In some embodiments, the compound has the structure of Formula (XXVII):

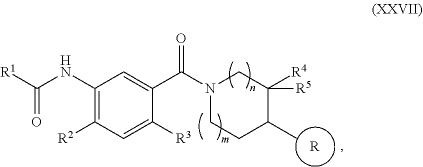

wherein R¹ is selected from the group consisting of C₁₋₆alkyl, fluorinated C₁₋₃alkyl, C₃₋₆cycloalkyl, —(C₁₋₂ alkyl)-C₃₋₆cycloalkyl, aryl, 5 to 6 membered heteroaryl, 9 to 10 membered heteroaryl, 4 to 6 membered saturated heterocyclyl and 9 to 10 membered saturated, partially unsaturated or benzo-fused heterocyclyl; wherein the C₃₋₆cycloalkyl, aryl, 5 to 6 membered heteroaryl, 9 to 10 membered heteroaryl, 4 to 6 membered saturated heterocyclyl, or 9 to 10 membered saturated, partially unsaturated or benzo-fused heterocyclyl is optionally substituted with one to three R⁰ substituents; wherein each R⁰ is independently selected from the group consisting of halogen, hydroxy, cyano, C₁₋₆alkyl, fluorinated C₁₋₂ alkyl, C₁₋₄alkoxy, —NR^{A}R^{B}, —C(O)—(C₁₋₄alkyl), —S—(C₁₋₄alkyl), —SO—(C₁₋₄alkyl), —SO₂—(C₁₋₄alkyl), —C₃₋₆cycloalkyl, —(C₁₋₂alkyl)-C₃₋₆cycloalkyl, —C(O)—C₃₋₆cycloalkyl, —(C₁₋₂alkyl)-phenyl and 5 to 6 membered saturated heterocyclyl; wherein the C₃₋₆cycloalkyl or 5 to 6 membered saturated heterocyclyl is optionally substituted with one to two substituents independently selected from the group consisting of C₁₋₄alkyl and hydroxy substituted C₁₋₂alkyl; wherein R^{A} is selected from the group consisting of hydrogen and C₁₋₄alkyl; and wherein R^{B} is selected from the group consisting of hydrogen, formyl, C₁₋₆alkyl, C₃₋₆cycloalkyl and 5 to 6 membered saturated heterocyclyl; wherein the R^{B} 5 to 6 membered saturated heterocyclyl is optionally substituted with C₁₋₄alkyl; R² is selected from the group consisting of halogen, hydroxy, cyano, C₁₋₄alkyl, fluorinated C₁₋₃ alkyl, C₁₋₄alkoxy, benzyloxy and —NR^{X}R^{Y}; wherein R^{X} is selected from the group consisting of hydrogen, C₁₋₄alkyl and —(C₂₋₄alkyl)-O—(C₁₋₂alkyl); and wherein R^{Y} is selected from the group consisting of hydrogen, C₁₋₄alkyl, —(C₂₋₄alkyl)-O—(C₁₋₂alkyl), C₃₋₆cycloalkyl and —C(O)—C₃₋₆cycloalkyl; R³ is selected from the group consisting of hydrogen, halogen, methyl and trifluoromethyl; n is an integer from 0 to 2; and m is an integer from 0 to 1; such that

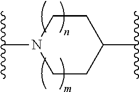

is selected from the group consisting of azetidin-1,3-diyl, pyrrolidin-1,3-diyl, piperidin-1,3-diyl, and piperidin-1,4-diyl; R⁴ is selected from the group consisting of hydrogen and C₁₋₃alkyl; R⁵ is selected from the group consisting of hydrogen, hydroxy and C₁₋₃alkyl; provided that when n is 0 and m is 0, such that

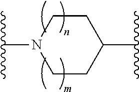

is azetidin-1,3-diyl, then R₅ is selected from the group consisting of hydrogen and C₁₋₃alkyl,

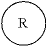

is selected from the group consisting of

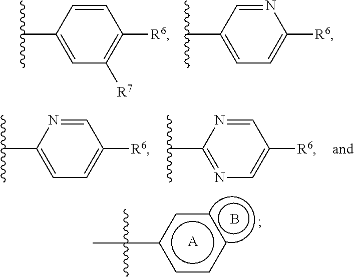

wherein R⁶ is selected from the group consisting of aryl, 5 to 6 membered heteroaryl and 9 to 10 membered heteroaryl; wherein the aryl, 5 to 6 membered heteroaryl or 9 to 10 membered heteroaryl is optionally substituted with one to three substituents independently 10 selected from the group consisting of halogen, cyano, C₁₋₄ alkyl, trifluoromethyl, hydroxy substituted C₁₋₃alkyl, C₁₋₄alkoxy, NR^{P}R^{Q}, —(C₁₋₂alkyl)-NR^{P}R^{Q}, C₃₋₆ cycloalkyl, —(C₁₋₂alkyl)-C₃₋₆cycloalkyl, 5 to 6 membered saturated heterocyclyl and 5 to 6 membered heretoaryl; wherein R^{P} and R^{Q} are each independently selected from the group consisting of hydrogen and C₁₋₄alkyl; wherein R⁷ is selected from the group consisting of hydrogen, halogen, cyano, C₁₋₄alkyl and trifluoromethyl; wherein

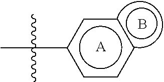

represents a 9 to 10 membered bicyclic, partially unsaturated or aromatic heterocyclic; and wherein the

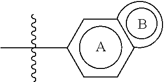

is optionally substituted with one to three substituents independently selected from the group consisting of halogen, oxo, cyano, C₁₋₄alkyl, trifluoromethyl, C₁₋₄ alkyoxy, NR^{S}R^{T} and cyclopropyl; wherein R^{S} and R^{T} are each independently selected from the group consisting of hydrogen and C₁₋₄alkyl; and stereoisomers, tautomers, and pharmaceutically acceptable salts thereof.

In some embodiments, R¹ is selected from the group consisting of C₁₋₆alkyl, fluorinated C₁₋₃alkyl, C₃₋₆cycloalkyl, aryl, 5 to 6 membered heteroaryl, 9 to 10 membered heteroaryl, 4 to 6 membered saturated heterocyclyl and 9 to 10 membered benzo-fused heterocyclyl; wherein the C₃₋₆cycloalkyl, aryl, 5 to 6 membered heteroaryl, 9 to 10 membered heteroaryl, 4 to 6 membered saturated heterocyclyl or 9 to 10 membered benzo-fused heterocyclyl is optionally substituted with one to three R⁰ substituents; wherein each R⁰ is independently selected from the group consisting of halogen, hydroxy, cyano, C₁₋₆ alkyl, fluorinated C₁₋₂alkyl, C₁₋₄alkoxy, —NR^{A}R^{B}, —C(O)—(C₁₋₄alkyl), —S—(C₁₋₄alkyl), —SO₂—(C₁₋₄alkyl), —C₃₋₆ cycloalkyl, —(C₁₋₂alkyl)-C₃₋₆cycloalkyl, —C(O)—C₃₋₆cycloalkyl, —(C₁₋₂alkyl)-phenyl and 5 to 6 membered saturated heterocyclyl; wherein the C₃₋₆cycloalkyl or 5 to 6 membered saturated heterocyclyl is optionally substituted with one to two substituents independently selected from the group consisting of C₁₋₄alkyl and 5 hydroxy substituted C₁₋₂alkyl; wherein R^{A} is selected from the group consisting of hydrogen and C₁₋₄ alkyl; and wherein R^{B} is selected from the group consisting of hydrogen, formyl, C₁₋₆alkyl, C₃₋₆cycloalkyl and 5 to 6 membered saturated, nitrogen containing heterocyclyl; wherein the R^{B} 5 to 6 membered saturated, nitrogen containing heterocyclyl is optionally substituted with C₁₋₄alkyl; R² is selected from the group consisting of halogen, hydroxy, cyano, C₁₋₄alkyl, fluorinated C₁₋₂alkyl, C₁₋₄alkoxy, benzyloxy and —NR^{X}R^{Y}; wherein R^{X} is selected from the group consisting of hydrogen, C₁₋₄alkyl and —(C₂₋₄alkyl)-O—(C₁₋₂alkyl); and wherein R^{Y} is selected from the group consisting of hydrogen, C₁₋₄alkyl, —(C₂₋₄alkyl)-O—(C₁₋₂ alkyl), C₃₋₆cycloalkyl and —C(O)—C₃₋₆cycloalkyl; R³ is selected from the group consisting of hydrogen, fluoro, chloro, bromo, methyl and trifluoromethyl; n is an integer from 0 to 1; and m is an integer from 0 to 1; such that

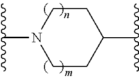

is selected from the group consisting of azetidin-1,3-diyl, pyrrolidin-1,3-diyl, and piperidin-1,4-diyl; R⁴ is selected from the group consisting of hydrogen and C₁₋₃alkyl, R⁵ is selected from the group consisting of hydrogen, hydroxy, and C₁₋₃alkyl; provided that when n is 0 and m is 0, such that

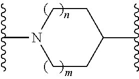

is azetidin-1,3-diyl, then R⁵ is selected from the group consisting of hydrogen and C₁₋₃alkyl;

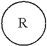

is selected from the group consisting of

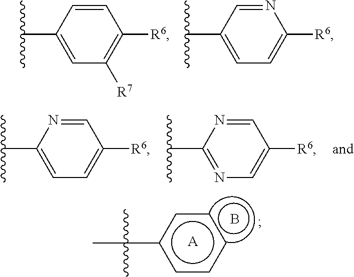

wherein R⁶ is selected from the group consisting of aryl, 5 to 6 membered heteroaryl and 9 to 10 membered heteroaryl; wherein the aryl, 5 to 5 6 membered heteroaryl or 9 to 10 membered heteroaryl is optionally substituted with one to two substituents independently selected from the group consisting of halogen, C₁₋₄alkyl, trifluoromethyl, hydroxy substituted C₁₋₂alkyl, C₁-4alkoxy, NR^{P}R^{Q}, —(C₁₋₂alkyl)-NR^{P}R^{Q}, C₃₋₆cycloalkyl, —(C₁₋₂alkyl)-C₃₋₆cycloalkyl, 5 to 6 membered saturated, nitrogen containing heterocyclyl and 5 to 6 membered nitrogen containing heretoaryl; wherein R^{P} and R^{Q} are each independently selected from the group consisting of hydrogen and C₁₋₄alkyl; wherein R⁷ is selected from the group consisting of hydrogen, fluoro, chloro, bromo, C₁₋₄alkyl and trifluoromethyl; wherein

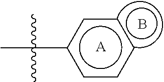

represents a 9 to 10 membered bicyclic, partially unsaturated or aromatic heterocyclyl, and wherein the

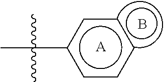

is optionally substituted with one to two substituents independently selected from the group consisting of halogen, oxo, cyano, C₁₋₄alkyl, trifluoromethyl, C₁₋₄ alkoxy, NR^{S}R^{T} and cyclopropyl; wherein R^{S} and R^{T} are each independently selected from the group consisting of hydrogen and C₁₋₄alkyl; and stereoisomers, tautomers and pharmaceutically acceptable salts thereof.

In some embodiments, R¹ is selected from the group consisting of C₂₋₅alkyl, fluorinated C₁₋₂alkyl, C₃₋₆cycloalkyl, phenyl, 4 to 6 membered saturated heterocyclyl, 5 to 6 membered heteroaryl, 9 to 10 membered heteroaryl and 1,3-benzodioxolyl; wherein the C₃₋₆cycloalkyl, phenyl, 4 to 6 membered saturated heterocyclyl, 5 to 6 membered heteroaryl or 9 to 10 membered heteroaryl is optionally substituted with one to three R⁰ substituents; wherein each R⁰ is independently selected from the group consisting of halogen, hydroxy, cyano, C₁₋₆alkyl, 5 fluorinated C₁₋₂alkyl, C₁₋₂alkoxy, NR^{A}R^{B}, —C(O)—(C₁₋₂ alkyl), —S—(C₁₋₂alkyl), C₅₋₆cycloalkyl, —C(O)—C₃cycloalkyl, —(C₁₋₂alkyl)-phenyl and 5 to 6 membered, saturated, nitrogen containing heterocyclyl; wherein the C₅₋₆cycloalkyl or 5 to 6 membered saturated, nitrogen containing heterocyclyl is optionally substituted with a substituent selected from the group consisting of C₁-2alkyl and —(C₁₋₂alkyl)-OH; wherein R^{A} is selected from the group consisting of hydrogen and C₁₋₂alkyl; and wherein R^{B} is selected from the group consisting of hydrogen, formyl, C₁₋₄alkyl, C₃₋₄ cycloalkyl and 6 membered, saturated, nitrogen containing heterocyclyl; wherein the R^{B} 6 membered saturated, nitrogen containing heterocyclyl is optionally substituted with C₁₋₂alkyl; R² is selected from the group consisting of halogen, hydroxy, C₁₋₂alkyl, C₁₋₂alkoxy, benzyloxy and —NR^{X}R^{Y}; wherein R^{X} is selected from the group consisting of hydrogen, C₁₋₃alkyl and -(C₂alkyl)-O—(C₁₋₂ alkyl); and wherein R^{Y} is selected from the group consisting of hydrogen, C₁₋₃alkyl, -(C₂alkyl)-O—(C₁₋₂ alkyl), C₃cycloalkyl and —C(O)—C₃cycloalkyl; R³ is hydrogen; n is an integer from 0 to 1; and m is an integer from 0 to 1; such that

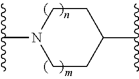

is selected from the group consisting of azetidin-1,3-diyl, pyrrolidin-1,3-diyl, and piperidin-1,4-diyl; R⁴ is selected from the group consisting of hydrogen and C₁₋₂alkyl; R⁵ is selected from the group consisting of hydrogen, hydroxy and C₁₋₂alkyl; provided that when n is 0 and m is 0, such that

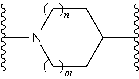

is azetidin-1,3-diyl, then R⁵ is selected from the group consisting of hydrogen and C₁₋₃alkyl;

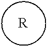

is selected from the group consisting of

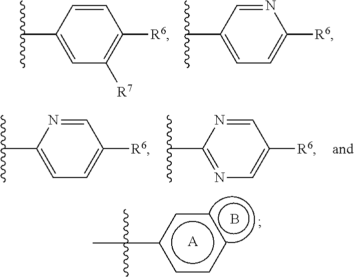

wherein R⁶ is selected from the group consisting of phenyl, 5 to 6 membered heteroaryl and 9 to 10 membered, nitrogen containing heteroaryl; 5 wherein the phenyl, 5 to 6 membered heteroaryl or 9 to 10 membered, nitrogen containing heteroaryl is optionally substituted with a substituent selected from the group consisting of halogen, C₁₋₄alkyl, —(C₁₋₂alkyl)-OH, C₁₋₂alkoxy, NR^{P}R^{Q}, —(C₁₋₂alkyl)-NR^{P}R^{Q}, C₃₋₄cycloalkyl, —(C₁₋₂alkyl)-C₃₋₄cycloalkyl, 6 membered saturated, nitrogen containing heterocyclyl and 6 membered, nitrogen containing heteroaryl; wherein R^{P} and R^{Q} are each independently selected from the group consisting of hydrogen and C₁₋₂alkyl; R⁷ is hydrogen; and wherein

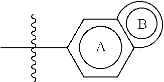

represents a 9 to 10 membered, bicyclic, partially unsaturated or aromatic, nitrogen containing heterocyclyl; wherein the optionally substituted with one to two substituents independently selected from the group consisting of oxo and C₁₋₂alkyl; and stereoisomers, tautomers and pharmaceutically acceptable salts thereof.

In some embodiments, R¹ is selected from the group consisting of t-butyl, n-pent-3-yl, isopropyl, 1-fluoro-ethyl, cyclopropyl, cyclobutyl, cyclopentyl, 4S-ethylcarbonyl-cyclopent-1S-yl, cyclohexyl, tetrahydropyran-4-yl, piperidin-4-yl, 1-methyl-piperidin-4-yl, 1-ethyl-piperidin-4-yl, 1-isopropyl-piperidin-4-yl, 1-(n-butyl)-piperidin-4-yl, 1-(1-methyl-n-pentyl)-piperidin-4-yl, 1-(n-pentyl)-piperidin-4-yl, 1-(2,2-dimethyl-propyl)-piperidin-4-yl, 1-isobutyl-piperidin-4-yl, 1-propyl-piperidin-4-yl, 1-isopentyl-piperidin-4-yl, 1-(n-hexyl)-piperidin-4-yl, 1-cyclobutyl-piperidin-4-yl, 1-cyclopentyl-piperidin-4-yl, 1-cyclohexyl-piperidin-4-yl, 1-(3-methyl-cyclopentyl)-piperidin-4-yl, 1-benzyl-piperidin-4-yl, tetrahydrofuran-2-yl, pyrrolidin-3-yl, pyrrolidin-2S-yl, pyrrolidin-2R-yl, 1-methyl-pyrrolidin-3R-yl, 1-methyl-pyrrolidin-3S-yl, 1-ethyl-pyrrolidin-3-yl, 1-propyl-pyrrolidin-3-yl, 1-isobutyl-pyrrolidin-3-yl, 1-(2,2-dimethyl-propyl)-pyrrolidin-3-yl, 1-isopropyl-pyrrolidin-3-yl, 1-(n-butyl)-pyrrolidin-3-yl, 1-(n-pentyl)-pyrrolidin-3-yl, 1-isopentyl-pyrrolidin-3-yl, 1-(1-methyl-n-pentyl)-pyrrolidin-3-yl, 1-(n-hexyl)-pyrrolidin-3-yl, 1-cyclobutyl-pyrrolidin-3-yl, 1-cyclopentyl-pyrrolidin-3-yl, 1-(3-methyl-cyclopentyl)-pyrrolidin-3-yl, 1-cyclohexyl-pyrrolidin-3-yl, 1-(cyclopropyl-carbonyl)-pyrrolidin-3-yl, azetidin-3-yl, 1-methyl-azetidin-3-yl, 1-ethyl-azetidin-3-yl, 1-isopropyl-azetidin-3-yl, 1-(n-propyl)-azetidin-3-yl, 1-(n-butyl)-azetidin-3-yl, 1-isobutyl-azetidin-3-yl, 1-isopentyl-azetidin-3-yl, 1-(n-pentyl)-azetidin-3-yl, 1-(2,2-dimethyl-propyl)-azetidin-3-yl, 1-(1-methyl-n-pentyl)-azetidin-3-yl, 1-(n-hexyl)-azetidin-3-yl, 1-cyclobutyl-azetidin-3-yl, 1-(3-methyl-cyclopentyl)-azetidin-3-yl, 1-cyclopentyl-azetidin-3-yl, 1-cyclohexyl-azetidin-3-yl, 1-(cyclopropyl-carbonyl)-azetidin-3-yl, phenyl, 2-chloro-phenyl, 3-chloro-phenyl, 4-chloro-phenyl, 2-fluoro-phenyl, 4-dichloro-phenyl, 2,4-dichloro-phenyl, 2,6-dichloro-phenyl, 3,4-dichloro-phenyl, 2,3,4-trifluoro-phenyl, 2,4-difluoro-phenyl, 2-fluoro-5-methyl-phenyl, 3-chloro-5-methoxy-phenyl, 2-fluoro-4-cyano-phenyl, 2-chloro-4-fluoro-phenyl, 4-isopropyl-phenyl, 2-methoxy-phenyl, 3-methoxy-phenyl, 2-methyl-4-fluoro-phenyl, 2-methyl-5-fluoro-phenyl, 3-hydroxy-4-methoxy-phenyl, 3-chloro-4-methoxy-phenyl, 4-methoxy-phenyl, 4-methylthio-phenyl, 2-trifluoromethyl-phenyl, 4-trifluoromethyl-phenyl, 4-cyano-phenyl, thiophen-2-yl, 3-chloro-thiophen-2-yl, 3-methyl-thiophen-2-yl, 5-methyl-thiophen-3-yl, thiazol-2-yl, thiazol-5-yl, 2-bromo-thiazol-2-yl, 4-t-butyl-thiazol-2-yl, pyridin-2-yl, pyridin-4-yl, 2-chloro-pyridin-3-yl, 4-chloro-pyridin-3-yl, 6-chloro-pyridin-3-yl, 3-fluoro-pyridin-4-yl, 5-bromo-pyridin-3-yl, 2-chloro-6-methoxy-pyridin-4-yl, 6-methyl-pyridin-4-yl, 6-trifluoromethyl-pyridin-2-yl, 6-methoxy-pyridin-3-yl, 5-(dimethylamino)-pyridin-2-yl, 6-(isopropyl-amino)-pyridin-3-yl, 6-(cyclobutyl-amino)-pyridin-3-yl, 6-(piperidin-1-yl)-pyridin-3-yl, 6-(morpholin-4-yl)-pyridin-3-yl, 6-(4-methyl-piperazin-1-yl)-pyridin-3-yl, 6-(N-methyl-N-(1-methyl-piperidin-4-yl)-amino-)-pyridin-3-yl, 6-(N-methyl-N-isopropyl-amino)-pyridin-3-yl, 6-(pyrrolidin-1-yl)-pyridin-3-yl, 6-(3S-hydroxymethyl-piperazin-1-yl)-pyridin-3-yl, 6-(3R-hydroxymethyl-piperazin-4-yl)-pyridin-3-yl, 6-(N-isopropyl-N-formyl)-pyridin-3-yl, 6-(dimethylamino)-pyridin-3-yl, 2-chloro-pyrimidin-5-yl, 2-(isopropyl-amino)-pyrimidin-5-yl, 2-(N-methyl-N-isopropyl-amino)-pyrimidin-5-yl, 2-(morpholin-4-yl)-pyrimidin-5-yl, 6-(morpholin-4-yl)-pyrimidin-5-yl, 2-(cyclobutyl-amino)-pyrimidin-5-yl, 1-methyl-imidazol-2-yl, quinolin-2-yl, indol-5-yl and 1,3-benzodioxol-5-yl; R² is selected from the group consisting of chloro, hydroxy, methyl, ethyl, methoxy, amino, methyl-amino, isopropyl-amino, (methoxyethyl)-amino, cyclopropyl-amino, (cyclopropylcarbonyl)-amino, N,N-dimethylamino, N-methyl-N-isopropyl-amino, N-methyl-N-(methoxyethyl)-amino, N-methyl-N-cyclopropyl-amino, N-(methoxyethyl)-N-(cyclopropylcarbonyl)-amino and benzyloxy; R³ is hydrogen; n is an integer from 0 to 1; and m is an integer from 0 to 1; such that

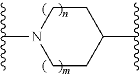

is selected from the group consisting of azetidin-1,3-diyl, pyrrolidin-1,3-diyl, and piperidin-1,4-diyl; R⁴ is selected from the group consisting of hydrogen and methyl; R⁵ is selected from the group consisting of hydrogen, hydroxy, trans-hydroxy, methyl, trans-methyl, and cis-methyl; provided that when n is 0 and m is 0, such that

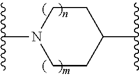

is azetidin-1,3-diyl, then R⁵ is selected from the group consisting of hydrogen, methyl, trans-methyl, and cis-methyl;

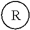

is selected from the group consisting of

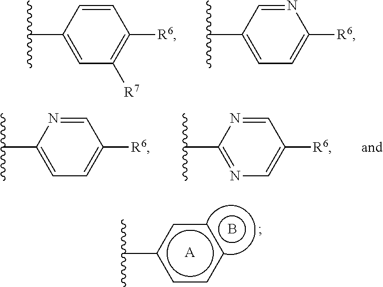

wherein R⁶ is selected from the group consisting of phenyl, 2-fluoro-phenyl, 3-fluoro-phenyl, 4-fluoro-phenyl, 2-methyl-phenyl, 3-methyl-phenyl, 4-methyl-phenyl, 2-methoxy-phenyl, 3-methoxy-phenyl, 4-methoxy-phenyl, thiophen-2-yl, thiophen-3-yl, furan-2-yl, furan-3-yl, isoxazol-4-yl, pyridin-3-yl, pyridin-4-yl, 2-amino-pyridin-3-yl, 3-amino-pyridin-4-yl, pyrazol-4-yl, 1-methyl-pyrazol-4-yl, 1-methyl-pyrazol-5-yl, 1-(tetrahydropyran-4-yl)-pyrazol-4-yl, 1-(cyclobutyl-methyl)-pyrazol-4-yl, 1,3-dimethyl-pyrazol-4-yl, 1-isopropyl-pyrazol-4-yl, 1-(2-hydroxyethyl)-pyrazol-4-yl, 1-cyclobutyl-pyrazol-4-yl, 1-(cyclopropyl)-pyrazol-4-yl, 1-(cyclopropyl-methyl)-pyrazol-4-yl, 1-(dimethylamino-ethyl)-pyrazol-4-yl, 1-(pyridin-3-yl)-pyrazol-4-yl, 1-(pyridin-4-yl)-pyrazol-4-yl, 1-methyl-indazol-6-yl, imidazol-1-yl, quinolin-4-yl, quinolin-5-yl and isoquinolin-6-yl; R⁷ is hydrogen; and wherein

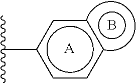

is selected from the group consisting of benzothiazol-6-yl, 2-oxo-benzothiazol-6-yl, 2-oxo-2,3,4-trihydro-quinolin-7-yl, isoquinolin-6-yl, isoquinolin-7-yl, 2-oxo-indolin-5-yl, 1-methyl-2-oxo-isoindol-5-yl, 1,7-dimethyl-isoindol-5-yl, 1-methyl-indazol-6-yl, imidazo[1,2-a]pyridine-6-yl and [1,2,4]triazolo[4,3-a]pyridine-6-yl; and stereoisomers, tautomers and pharmaceutically acceptable salts thereof.

In some embodiments, R¹ is selected from the group consisting of n-pent-3-yl, cyclopropyl, cyclobutyl, cyclopentyl, 4S-ethylcarbonyl-cyclopent-1S-yl, cyclohexyl, tetrahydropyran-4-yl, piperidin-4-yl, 1-methyl-piperidin-4-yl, 1-ethyl-piperidin-4-yl, 1-isopropyl-piperidin-4-yl, 1-(1-methyl-n-pentyl)-piperidin-4-yl, 1-(n-pentyl)-piperidin-4-yl, 1-(2,2-dimethyl-propyl)-piperidin-4-yl, 1-isobutyl-piperidin-4-yl, 1-propyl-piperidin-4-yl, 1-isopentyl-piperidin-4-yl, 1-(n-hexyl)-piperidin-4-yl, 1-cyclobutyl-piperidin-4-yl, 1-cyclopentyl-piperidin-4-yl, 1-cyclohexyl-piperidin-4-yl, 1-benzyl-piperidin-4-yl, pyrrolidin-3-yl, 1-propyl-pyrrolidin-3-yl, 1-isobutyl-pyrrolidin-3-yl, 1-isopentyl-pyrrolidin-3-yl, 1-(3-methyl-cyclopentyl)-pyrrolidin-3-yl, 1-(cyclopropyl-carbonyl)-pyrrolidin-3-yl, 1-methyl-azetidin-3-yl, 1-(n-butyl)-azetidin-3-yl, 1-isobutyl-azetidin-3-yl, 1-isopentyl-azetidin-3-yl, 1-(2,2-dimethyl-propyl)-azetidin-3-yl, 1-cyclobutyl-azetidin-3-yl, 1-cyclohexyl-azetidin-3-yl, 1-(cyclopropyl-carbonyl)-azetidin-3-yl, phenyl, 2-chloro-phenyl, 3-chloro-phenyl, 4-chloro-phenyl, 2-fluoro-phenyl, 4-dichloro-phenyl, 2,4-dichloro-phenyl, 3,4-dichloro-phenyl, 2,3,4-trifluoro-phenyl, 2,4-difluoro-phenyl, 2-fluoro-4-cyano-phenyl, 2-chloro-4-fluoro-phenyl, 4-isopropyl-phenyl, 3-methoxy-phenyl, 2-methyl-5-fluoro-phenyl, 3-hydroxy-4-methoxy-phenyl, 3-chloro-4-methoxy-phenyl, 4-methoxy-phenyl, 4-methylthio-phenyl, 4-trifluoromethyl-phenyl, 4-cyano-phenyl, thiophen-2-yl, 3-chloro-thiophen-2-yl, 3-methyl-thiophen-2-yl, 5-methyl-thiophen-3-yl, thiazol-5-yl, 2-bromo-thiazol-2-yl, pyridin-2-yl, pyridin-4-yl, 2-chloro-pyridin-3-yl, 6-chloro-pyridin-3-yl, 3-fluoro-pyridin-4-yl, 2-chloro-6-methoxy-pyridin-4-yl, 6-methyl-pyridin-4-yl, 6-methoxy-pyridin-3-yl, 5-(dimethylamino)-pyridin-2-yl, 6-(isopropyl-amino)-pyridin-3-yl, 6-(cyclobutyl-amino)-pyridin-3-yl, 6-(piperidin-1-yl)-pyridin-3-yl, 6-(morpholin-4-yl)-pyridin-3-yl, 6-(4-methyl-piperazin-1-yl)-pyridin-3-yl, 6-(N-methyl-N-(1-methyl-piperidin-4-yl)-amino)-pyridin-3-yl, 6-(N-methyl-N-isopropyl-amino)-pyridin-3-yl, 6-(pyrrolidin-1-yl)-pyridin-3-yl, 6-(3S-hydroxymethyl-piperazin-1-yl)-pyridin-3-yl, 6-(3R-hydroxymethyl-piperazin-4-yl)-pyridin-3-yl, 2-chloro-pyrimidin-5-yl, 2-(isopropyl-amino)-pyrimidin-5-yl, 2-(N-methyl-N-isopropyl-amino)-pyrimidin-5-yl, 2-(morpholin-4-yl)-pyrimidin-5-yl, 6-(morpholin-4-yl)-pyrimidin-5-yl, 2-(cyclobutyl-amino)-pyrimidin-5-yl, quinolin-2-yl, indol-5-yl and 1,3-benzodioxol-5-yl; R² is selected from the group consisting of chloro, hydroxy, methyl, ethyl, methoxy, benzyloxy, methylamino, (methoxyethyl)amino, dimethylamino and N—methyl-N-cyclopropyl-amino; R³ is hydrogen; n is 0; and m is 0; such that

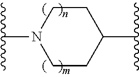

is azetidin-1,3-diyl; alternatively, n is 1; and m is 1; such that

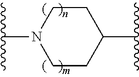

is piperidin-1,4-diyl; R⁴ is selected from the group consisting of hydrogen and methyl; R⁵ is selected from the group consisting of hydrogen, methyl, and trans-methyl;

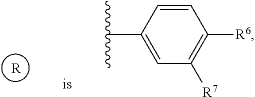

R⁶ is selected from the group consisting of furan-3-yl, thiophen-3-yl, pyridin-3-yl, pyridin-4-yl, 2-amino-pyridin-3-yl, 3-amino-pyridin-4-yl, imidazol-1-yl, isoxazol-4-yl, pyrazol-4-yl, 1-methyl-pyrazol-4-yl, 1-isopropyl-pyrazol-4-yl, 1-(2-hydroxyethyl)-pyrazol-4-yl, 1-cyclopropyl-pyrazol-4-yl, 1-cyclobutyl-pyrazol-4-yl, 1-(cyclopropyl-methyl)-pyrazol-4-yl, 1,3-dimethyl-pyrazol-4-yl, 1-(pyridin-3-yl)-pyrazol-4-yl, 1-(pyridin-4-yl)-pyrazol-4-yl, 1-methyl-pyrazol-5-yl, quinolin-4-yl, quinolin-5-yl, isoquinolin-6-yl and 1-methyl-indazol-6-yl; and R⁷ is hydrogen; and stereoisomers, tautomers and pharmaceutically acceptable salts thereof.

In some embodiments, R¹ is selected from the group consisting of n-pent-3-yl, cyclopropyl, cyclohexyl, 1-isopropyl-piperidin-4-yl, 1-isobutyl-piperidin-4-yl, 1-cyclopentyl-piperidin-4-yl, 1-cyclohexyl-piperidin-4-yl, 1-methyl-azetidin-3-yl, phenyl, 3-chloro-phenyl, 4-chloro-phenyl, 2-fluoro-phenyl, 2,4-dichloro-phenyl, 2-fluoro-4-cyano-phenyl, 3-methoxy-phenyl, 2-methyl-5-fluoro-phenyl, 3-hydroxy-4-methoxy-phenyl, 4-methoxy-phenyl, 4-methylthio-phenyl, 4-trifluoromethyl-phenyl, 3-chloro-thiophen-2-yl, pyridin-4-yl, 6-chloro-pyridin-3-yl, 3-fluoro-pyridin-4-yl, 6-methyl-pyridin-4-yl, 6-methoxy-pyridin-3-yl, 6-(isopropyl-amino)-pyridin-3-yl, 6-(cyclobutyl-amino)-pyridin-3-yl, 6-(piperidin-1-yl)-pyridin-3-yl, 6-(morpholin-4-yl)-pyridin-3-yl, 6-(4-methyl-piperazin-1-yl)-pyridin-3-yl, 6-(N-methyl-N-(1-methyl-piperidin-4-yl)-amino)-pyridin-3-yl, 6-(N-methyl-N-isopropyl-amino)-pyridin-3-yl, 6-(pyrrolidin-1-yl)-pyridin-3-yl, 6-(3S-hydroxymethyl-piperazin-1-yl)-pyridin-3-yl, 6-(3R-hydroxymethyl-piperazin-4-yl)-pyridin-3-yl, 2-chloro-pyrimidin-5-yl, 2-(isopropyl-amino)-pyrimidin-5-yl, 2-(N-methyl-N-isopropyl-amino)-pyrimidin-5-yl, 2-(morpholin-4-yl)-pyrimidin-5-yl, 6-(morpholin-4-yl)-pyrimidin-5-yl and 2-(cyclobutyl-amino)-pyrimidin-5-yl; R² is selected from the group consisting of chloro, methyl, ethyl and methoxy; R³ is hydrogen; n is 0; and m is 0; such that

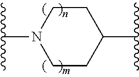

is azetidin-1,3-diyl; alternatively, n is 1; and m is 1; such that

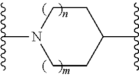

is piperidin-1,4-diyl; R⁴ is hydrogen; R⁵ is selected from the group consisting of hydrogen and trans-methyl;

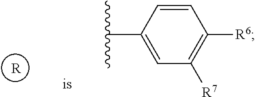

R⁶ is selected from the group consisting of pyridin-4-yl, 2-amino-pyridin-3-yl, 3-amino-pyridin-4-yl, imidazol-1-yl, isoxazol-4-yl, pyrazol-4-yl, 1-methyl-pyrazol-4-yl, 1-(pyridin-4-yl)-pyrazol-4-yl, 1-methyl-pyrazol-5-yl and quinolin-4-yl; and R⁷ is hydrogen; and stereoisomers, tautomers and pharmaceutically acceptable salts thereof.

In some embodiments, R¹ is selected from the group consisting of 1-methyl-azetidin-3-yl, 1-(n-butyl)-piperidin-4-yl, 1-(2,2-dimethyl-propyl)-piperidin-4-yl, 1-isopentyl-piperidin-4-yl, 1-cyclobutyl-piperidin-4-yl, 1-cyclopentyl-piperidin-4-yl, 1-cyclohexyl-piperidin-4-yl, 4-methylthio-phenyl, 2-fluoro-4-cyano-phenyl, 3-fluoro-pyridin-4-yl, 6-(3S-hydroxymethyl-piperidin-1-yl)-pyridin-3-yl, 6-(isopropyl-amino)-pyridin-3-yl, 6-(cyclobutyl-amino)-pyridin-3-yl, 6-(N-methyl-N-isopropyl-amino)-pyridin-3-yl, 6-(N-methyl-N-(1-methyl-piperidin-4-yl)-amino)-pyridin-3-yl, 6-(morpholin-4-yl)-pyridin-3-yl, 6-(4-methyl-piperazin-1-yl)-pyridin-3-yl, 2-(isopropyl-amino)-pyrimidin-5-yl, 2-(morpholin-4-yl)-pyrimidin-5-yl, 2-(cyclobutyl-amino)-pyrimidin-5-yl and indol-5-yl; R² is methyl; R³ is hydrogen; n is 0; and m is 0; such that

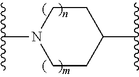

is azetidin-1,3-diyl; alternatively, n is 1; and m is 1; such that

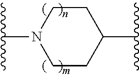

is piperidin-1,4-diyl; R⁴ is hydrogen; R⁵ is hydrogen;

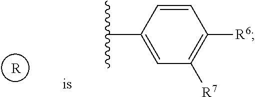

R⁶ is selected from the group consisting of pyridin-4-yl, 3-amino-pyridin-4-yl, 1-methyl-pyrazol-4-yl, 1-(2-hydroxyethyl)-pyrazol-4-yl, 1-cyclopropyl-pyrazol-4-yl, 1-methyl-pyrazol-5-yl and quinolin-4-yl; and R⁷ is hydrogen; and stereoisomers, tautomers and pharmaceutically acceptable salts thereof.

In some embodiments, R¹ is selected from the group consisting of cyclopropyl, 6-chloro-pyridin-3-yl, 6-(isopropyl-amino)-pyridin-3-yl, 6-(N-methyl-N-isopropyl-amino)-pyridin-3-yl and 6-(morpholin-4-yl)-pyridin-3-yl; R² is selected from the group consisting of methyl, amino, methylamino, isopropylamino, (methoxyethyl)amino, cyclopropylamino, dimethylamino and N-methyl-N-cyclopropyl-amino; R³ is hydrogen; n is 0; and m is 0; such that

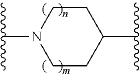

is azetidin-1,3-diyl; alternatively, n is 1; and m is 1; such that

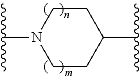

is piperidin-1,4-diyl; R⁴ is hydrogen; R⁵ is hydrogen;

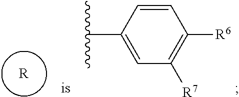

R⁶ is 1-methyl-pyrazol-4-yl; and R⁷ is hydrogen; and stereoisomers, tautomers and pharmaceutically acceptable salts thereof.

In some embodiments, R¹ is selected from the group consisting of t-butyl, n-pent-3-yl, isopropyl, 1-fluoro-ethyl, cyclopropyl, cyclobutyl, cyclopentyl, 4S-ethylcarbonyl-cyclopent-1S-yl, cyclohexyl, tetrahydropyran-4-yl, piperidin-4-yl, 1-methyl-piperidin-4-yl, 1-ethyl-piperidin-4-yl, 1-isopropyl-piperidin-4-yl, 1-(n-butyl)-piperidin-4-yl, 1-(1-methyl-n-pentyl)-piperidin-4-yl, 1-(n-pentyl)-piperidin-4-yl, 1-(2,2-dimethyl-propyl)-piperidin-4-yl, 1-isobutyl-piperidin-4-yl, 1-propyl-piperidin-4-yl, 1-isopentyl-piperidin-4-yl, 1-(n-hexyl)-piperidin-4-yl, 1-cyclobutyl-piperidin-4-yl, 1-cyclopentyl-piperidin-4-yl, 1-cyclohexyl-piperidin-4-yl, 1-(3-methyl-cyclopentyl)-piperidin-4-yl, 1-benzyl-piperidin-4-yl, tetrahydrofuran-2-yl, pyrrolidin-3-yl, pyrrolidin-2S-yl, pyrrolidin-2R-yl, 1-methyl-pyrrolidin-3R-yl, 1-methyl-pyrrolidin-3S-yl, 1-ethyl-pyrrolidin-3-yl, 1-propyl-pyrrolidin-3-yl, 1-isobutyl-pyrrolidin-3-yl, 1-(2,2-dimethyl-propyl)-pyrrolidin-3-yl, 1-isopropyl-pyrrolidin-3-yl, 1-(n-butyl)-pyrrolidin-3-yl, 1-(n-pentyl)-pyrrolidin-3-yl, 1-isopentyl-pyrrolidin-3-yl, 1-(1-methyl-n-pentyl)-pyrrolidin-3-yl, 1-(n-hexyl)-pyrrolidin-3-yl, 1-cyclobutyl-pyrrolidin-3-yl, 1-cyclopentyl-pyrrolidin-3-yl, 1-(3-methyl-cyclopentyl)-pyrrolidin-3-yl, 1-cyclohexyl-pyrrolidin-3-yl, 1-(cyclopropyl-carbonyl)-pyrrolidin-3-yl, azetidin-3-yl, 1-methyl-azetidin-3-yl, 1-ethyl-azetidin-3-yl, 1-isopropyl-azetidin-3-yl, 1-(n-propyl)-azetidin-3-yl, 1-(n-butyl)-azetidin-3-yl, 1-isobutyl-azetidin-3-yl, 1-isopentyl-azetidin-3-yl, 1-(n-pentyl)-azetidin-3-yl, 1-(2,2-dimethyl-propyl)-azetidin-3-yl, 1-(1-methyl-n-pentyl)-azetidin-3-yl, 1-(n-hexyl)-azetidin-3-yl, 1-cyclobutyl-azetidin-3-yl, 1-(3-methyl-cyclopentyl)-azetidin-3-yl, 1-cyclopentyl-azetidin-3-yl, 1-cyclohexyl-azetidin-3-yl, 1-(cyclopropyl-carbonyl)-azetidin-3-yl, phenyl, 2-chloro-phenyl, 3-chloro-phenyl, 4-chloro-phenyl, 2-fluoro-phenyl, 4-dichloro-phenyl, 2,4-dichloro-phenyl, 2,6-dichloro-phenyl, 3,4-dichloro-phenyl, 2,3,4-trifluoro-phenyl, 2,4-difluoro-phenyl, 2-fluoro-5-methyl-phenyl, 3-chloro-5-methoxy-phenyl, 2-fluoro-4-cyano-phenyl, 2-chloro-4-fluoro-phenyl, 4-isopropyl-phenyl, 2-methoxy-phenyl, 3-methoxy-phenyl, 2-methyl-4-fluoro-phenyl, 2-methyl-5-fluoro-phenyl, 3-hydroxy-4-methoxy-phenyl, 3-chloro-4-methoxy-phenyl, 4-methoxy-phenyl, 4-methylthio-phenyl, 2-trifluoromethyl-phenyl, 4-trifluoromethyl-phenyl, 4-cyano-phenyl, thiophen-2-yl, 3-chloro-thiophen-2-yl, 3-methyl-thiophen-2-yl, 5-methyl-thiophen-3-yl, thiazol-2-yl, thiazol-5-yl, 2-bromo-thiazol-2-yl, 4-t-butyl-thiazol-2-yl, pyridin-2-yl, pyridin-4-yl, 2-chloro-pyridin-3-yl, 4-chloro-pyridin-3-yl, 6-chloro-pyridin-3-yl, 3-fluoro-pyridin-4-yl, 5-bromo-pyridin-3-yl, 2-chloro-6-methoxy-pyridin-4-yl, 6-methyl-pyridin-4-yl, 6-trifluoromethyl-pyridin-2-yl, 6-methoxy-pyridin-3-yl, 5-(dimethylamino)-pyridin-2-yl, 6-(isopropyl-amino)-pyridin-3-yl, 6-(cyclobutyl-amino)-pyridin-3-yl, 6-(piperidin-1-yl)-pyridin-3-yl, 6-(morpholin-4-yl)-pyridin-3-yl, 6-(4-methyl-piperazin-1-yl)-pyridin-3-yl, 6-(N-methyl-N-(1-methyl-piperidin-4-yl)-amino-)-pyridin-3-yl, 6-(N-methyl-N-isopropyl-amino)-pyridin-3-yl, 6-(pyrrolidin-1-yl)-pyridin-3-yl, 6-(3S-hydroxymethyl-piperazin-1-yl)-pyridin-3-yl, 6-(3R-hydroxymethyl-piperazin-4-yl)-pyridin-3-yl, 6-(N-isopropyl-N-formyl)-pyridin-3-yl, 6-(dimethylamino)-pyridin-3-yl, 2-chloro-pyrimidin-5-yl, 2-(isopropyl-amino)-pyrimidin-5-yl, 2-(N-methyl-N-isopropyl-amino)-pyrimidin-5-yl, 2-(morpholin-4-yl)-pyrimidin-5-yl, 6-(morpholin-4-yl)-pyrimidin-5-yl, 2-(cyclobutyl-amino)-pyrimidin-5-yl, 1-methyl-imidazol-2-yl, quinolin-2-yl, indol-5-yl and 1,3-benzodioxol-5-yl; R² is selected from the group consisting of chloro, hydroxy, methyl, ethyl, methoxy, amino, methyl-amino, isopropyl-amino, (methoxyethyl)-amino, cyclopropyl-amino, (cyclopropylcarbonyl)-amino, N,N-dimethylamino, N-methyl-N-isopropyl-amino, N-methyl-N-(methoxyethyl)-amino, N-methyl-N-cyclopropyl-amino, N-(methoxyethyl)-N-(cyclopropylcarbonyl)-amino and benzyloxy; R³ is hydrogen; n is an integer from 0 to 1; and m is an integer from 0 to 1; such that

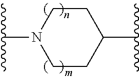

is selected from the group consisting of azetidin-1,3-diyl, pyrrolidin-1,3-diyl, and piperidin-1,4-diyl; R⁴ is selected from the group consisting of hydrogen and methyl; R⁵ is selected from the group consisting of hydrogen, hydroxy, methyl, trans-methyl, and cis-methyl; provided that when n is 0 and m is 0, such that

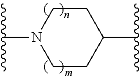

is azetidin-1,3-diyl, then R⁵ is selected from the group consisting of hydrogen, methyl, trans-methyl, and cis-methyl;

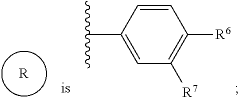

R⁶ is selected from the group consisting of phenyl, 2-fluoro-phenyl, 3-fluoro-phenyl, 4-fluoro-phenyl, 2-methyl-phenyl, 3-methyl-phenyl, 4-methyl-phenyl, 2-methoxy-phenyl, 3-methoxy-phenyl, 4-methoxy-phenyl, thiophen-2-yl, thiophen-3-yl, furan-2-yl, furan-3-yl, isoxazol-4-yl, pyridin-3-yl, pyridin-4-yl, 2-amino-pyridin-3-yl, 3-amino-pyridin-4-yl, pyrazol-4-yl, 1-methyl-pyrazol-4-yl, 1-methyl-pyrazol-5-yl, 1-(tetrahydropyran-4-yl)-pyrazol-4-yl, 1-(cyclobutyl-methyl)-pyrazol-4-yl, 1,3-dimethyl-pyrazol-4-yl, 1-isopropyl-pyrazol-4-yl, 1-(2-hydroxyethyl)-pyrazol-4-yl, 1-cyclobutyl-pyrazol-4-yl, 1-(cyclopropyl)-pyrazol-4-yl, 1-(cyclopropyl-methyl)-pyrazol-4-yl, 1-(dimethylamino-ethyl)-pyrazol-4-yl, 1-(pyridin-3-yl)-pyrazol-4-yl, 1-(pyridin-4-yl)-pyrazol-4-yl, 1-methyl-indazol-6-yl, imidazol-1-yl, quinolin-4-yl, quinolin-5-yl and isoquinolin-6-yl; and R⁷ is hydrogen; and stereoisomers, tautomers and pharmaceutically acceptable salts thereof.

In some embodiments, R¹ is selected from the group consisting of 6-chloro-pyridin-3-yl and 6-(isopropylamino)-pyridin-3-yl; R² is methyl; R³ is hydrogen; n is 1; and m is 1; such that

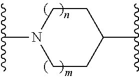

is piperidin-1,4-diyl; R⁴ is hydrogen; R⁵ is hydrogen;

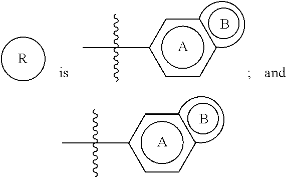

is selected from the group consisting of benzothiazol-6-yl, 2-oxo-benzothiazol-6-yl, 2-oxo-2,3,4-trihydro-quinolin-7-yl, isoquinolin-6-yl, isoquinolin-7-yl, 2-oxo-indolin-5-yl, 1-methyl-2-oxo-isoindol-5-yl, 1,7-dimethyl-isoindol-5-yl, 1-methyl-indazol-6-yl, imidazo[1,2-a]pyridine-6-yl and [1,2,4]triazolo[4,3-a]pyridine-6-yl; and stereoisomers, tautomers and pharmaceutically acceptable salts thereof.

In some embodiments, R¹ is selected from the group consisting of 6-chloro-pyridin-3-yl and 6-(isopropylamino)-pyridin-3-yl; R² is methyl; R³ is hydrogen; n is 1; and m is 1; such that

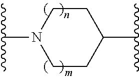

is piperidin-1,4-diyl; R⁴ is hydrogen; R⁵ is hydrogen;

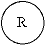

is selected from the group consisting of

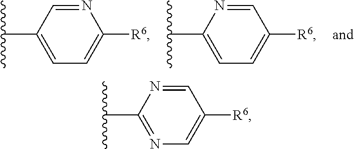

and R⁶ is 1-methyl-pyrazol-4-yl; and stereoisomers, tautomers, and pharmaceutically acceptable salts thereof.

In some embodiments, R¹ is selected from the group consisting of C₁₋₆alkyl, fluorinated C₁₋₃alkyl, C₃₋₆cycloalkyl, aryl, 5 to 6 membered heteroaryl, 9 to 10 membered heteroaryl, 4 to 6 membered saturated heterocyclyl and 9 to 10 membered benzo-fused heterocyclyl; wherein the C₃₋₆cycloalkyl aryl, 5 to 6 membered heteroaryl, 9 to 10 membered heteroaryl, 4 to 6 membered saturated heterocyclyl or 9 to 10 membered benzo-fused heterocyclyl is optionally substituted with one to three R⁰ substituents; wherein each R⁰ is independently selected from the group consisting of halogen, hydroxy, cyano, C₁₋₆ alkyl, fluorinated C₁₋₂alkyl, C₁₋₄alkoxy, —NR^{A}R^{B}, —C(O)—(C₁₋₄alkyl), —S—(C₁₋₄alkyl), —SO₂—(C₁₋₄alkyl), —C₃₋₆ cycloalkyl, —(C₁₋₂alkyl)-C₃₋₆cycloalkyl, —C(O)—C₃₋₆cycloalkyl, —(C₁₋₂alkyl)-phenyl and 5 to 6 membered saturated heterocyclyl; wherein the C₃₋₆cycloalkyl or 5 to 6 membered saturated heterocyclyl is optionally substituted with one to two substituents independently selected from the group consisting of C₁₋₄alkyl and hydroxy substituted C₁₋₂alkyl; wherein R^{A} is selected from the group consisting of hydrogen and C₁₋₄alkyl; and wherein R^{B} is selected from the group consisting of hydrogen, formyl, C₁₋₆alkyl, C₃₋₆cycloalkyl and 5 to 6 membered saturated, nitrogen containing heterocyclyl; wherein the R^{B} 5 to 6 membered saturated, nitrogen containing heterocyclyl is optionally substituted with C₁₋₄alkyl.

In another embodiment, the present invention is directed to compounds of formula (XXVII) wherein R¹ is selected from the group consisting of C₂₋₅alkyl, fluorinated C₁₋₂alkyl, C₃₋₆cycloalkyl, phenyl, 4 to 6 membered saturated heterocyclyl, 5 to 6 membered heteroaryl, 9 to 10 membered heteroaryl and 1,3-benzodioxolyl; wherein the C₃₋₆cycloalkyl, phenyl, 4 to 6 membered saturated heterocyclyl, 5 to 6 membered heteroaryl or 9 to 10 membered heteroaryl is optionally substituted with one to three R⁰ substituents; wherein each R⁰ is independently selected from the group consisting of halogen, hydroxy, cyano, C₁₋₆alkyl, fluorinated C₁₋₂alkyl, C₁₋₂alkoxy, NR^{A}R^{B}, —C(O)—(C₁₋₂alkyl), —S—(C₁₋₂alkyl), C₅₋₆cycloalkyl, —C(O)—C₃cycloalkyl, —(C₁₋₂alkyl)-phenyl and 5 to 6 membered, saturated, nitrogen containing heterocyclyl; wherein the C₅₋₆cycloalkyl or 5 to 6 membered saturated, nitrogen containing heterocyclyl is optionally substituted with a substituent selected from the group consisting of C₁₋₂alkyl and —(C₁₋₂alkyl)-OH; wherein R^{A} is selected from the group consisting of hydrogen and C₁₋₂alkyl; and wherein R^{B} is selected from the group consisting of hydrogen, formyl, C₁₋₄alkyl, C₃₋₄cycloalkyl and 6 membered, saturated, nitrogen containing heterocyclyl; wherein the R^{B} 6 membered saturated, nitrogen containing heterocyclyl is optionally substituted with C₁₋₂alkyl.

In another embodiment, R¹ is selected from the group consisting of t-butyl, n-pent-3-yl, isopropyl, 1-fluoro-ethyl, cyclopropyl, cyclobutyl, cyclopentyl, 4S-ethylcarbonyl-cyclopent-1S-yl, cyclohexyl, tetrahydropyran-4-yl, piperidin-4-yl, 1-methyl-piperidin-4-yl, 1-ethyl-piperidin-4-yl, 1-isopropyl-piperidin-4-yl, 1-(n-butyl)-piperidin-4-yl, 1-(1-methyl-n-pentyl)-piperidin-4-yl, 1-(n-pentyl)-piperidin-10 4-yl, 1-(2,2-dimethyl-propyl)-piperidin-4-yl, 1-isobutyl-piperidin-4-yl, 1-propyl-piperidin-4-yl, 1-isopentyl-piperidin-4-yl, 1-(n-hexyl)-piperidin-4-yl, 1-cyclobutyl-piperidin-4-yl, 1-cyclopentyl-piperidin-4-yl, 1-cyclohexyl-piperidin-4-yl, 1-(3-methyl-cyclopentyl)-piperidin-4-yl, 1-benzyl-piperidin-4-yl, tetrahydrofuran-2-yl, pyrrolidin-3-yl, pyrrolidin-2S-yl, pyrrolidin-2R-yl, 1-methyl-pyrrolidin-3R-yl, 1-methyl-pyrrolidin-3S-yl, 1-ethyl-pyrrolidin-3-yl, 1-propyl-pyrrolidin-3-yl, 1-isobutyl-pyrrolidin-3-yl, 1-(2,2-dimethyl-propyl)-pyrrolidin-3-yl, 1-isopropyl-pyrrolidin-3-yl, 1-(n-butyl)-pyrrolidin-3-yl, 1-(n-pentyl)-pyrrolidin-3-yl, 1-isopentyl-pyrrolidin-3-yl, 1-(1-methyl-n-pentyl)-pyrrolidin-3-yl, 1-(n-hexyl)-pyrrolidin-3-yl, 1-cyclobutyl-pyrrolidin-3-yl, 1-cyclopentyl-pyrrolidin-3-yl, 1-(3-methyl-cyclopentyl)-pyrrolidin-3-yl, 1-cyclohexyl-pyrrolidin-3-yl, 1-(cyclopropyl-carbonyl)-pyrrolidin-3-yl, azetidin-3-yl, 1-methyl-azetidin-3-yl, 1-ethyl-azetidin-3-yl, 1-isopropyl-azetidin-3-yl, 1-(n-propyl)-azetidin-3-yl, 1-(n-butyl)-azetidin-3-yl, 1-isobutyl-azetidin-3-yl, 1-isopentyl-azetidin-3-yl, 1-(n-pentyl)-azetidin-3-yl, 1-(2,2-dimethyl-propyl)-azetidin-3-yl, 1-(1-methyl-n-pentyl)-azetidin-3-yl, 1-(n-hexyl)-azetidin-3-yl, 1-cyclobutyl-azetidin-3-yl, 1-(3-methyl-cyclopentyl)-azetidin-3-yl, 1-cyclopentyl-azetidin-3-yl, 1-cyclohexyl-azetidin-3-yl, 1-(cyclopropyl-carbonyl)-azetidin-3-yl, phenyl, 2-chloro-phenyl, 3-chloro-phenyl, 4-chloro-phenyl, 2-fluoro-phenyl, 4-dichloro-phenyl, 2,4-dichloro-phenyl, 2,6-dichloro-phenyl, 3,4-dichloro-phenyl, 2,3,4-trifluoro-phenyl, 2,4-difluoro-phenyl, 2-fluoro-5-methyl-phenyl, 3-chloro-5-methoxy-phenyl, 2-fluoro-4-cyano-phenyl, 2-chloro-4-fluoro-phenyl, 4-isopropyl-phenyl, 2-methoxy-phenyl, 3-methoxy-phenyl, 2-methyl-4-fluoro-phenyl, 2-methyl-5-fluoro-phenyl, 3-hydroxy-4-methoxy-phenyl, 3-chloro-4-methoxy-phenyl, 4-methoxy-phenyl, 4-methylthio-phenyl, 2-trifluoromethyl-phenyl, 4-trifluoromethyl-phenyl, 4-cyano-phenyl, thiophen-2-yl, 3-chloro-thiophen-2-yl, 3-methyl-thiophen-2-yl, 5-methyl-thiophen-3-yl, thiazol-2-yl, thiazol-5-yl, 2-bromo-thiazol-2-yl, 4-t-butyl-thiazol-2-yl, pyridin-2-yl, pyridin-4-yl, 2-chloro-pyridin-3-yl, 4-chloro-pyridin-3-yl, 6-chloro-pyridin-3-yl, 3-fluoro-pyridin-4-yl, 5-bromo-pyridin-3-yl, 2-chloro-6-methoxy-pyridin-4-yl, 6-methyl-pyridin-4-yl, 6-trifluoromethyl-pyridin-2-yl, 6-methoxy-pyridin-3-yl, 5-(dimethylamino)-pyridin-2-yl, 6-(isopropyl-amino)-pyridin-3-yl, 6-(cyclobutyl-amino)-pyridin-3-yl, 6-(piperidin-1-yl)-pyridin-3-yl, 6-(morpholin-4-yl)-pyridin-3-yl, 6-(4-methyl-piperazin-1-yl)-pyridin-3-yl, 6-(N-methyl-N-(1-methyl-piperidin-4-yl)-amino-)-pyridin-3-yl, 6-(N-methyl-N-isopropyl-amino)-pyridin-3-yl, 6-(pyrrolidin-1-yl)-pyridin-3-yl, 6-(3S-hydroxymethyl-piperazin-1-yl)-pyridin-3-yl, 6-(3R-hydroxymethyl-piperazin-4-yl)-pyridin-3-yl, 6-(N-isopropyl-N-formyl)-pyridin-3-yl, 6-(dimethylamino)-pyridin-3-yl, 2-chloro-pyrimidin-5-yl, 2-(isopropyl-amino)-pyrimidin-5-yl, 2-(N-methyl-N-isopropyl-amino)-pyrimidin-5-yl, 2-(morpholin-4-yl)-pyrimidin-5-yl, 6-(morpholin-4-yl)-pyrimidin-5-yl, 2-(cyclobutyl-amino)-pyrimidin-5-yl, 1-methyl-imidazol-2-yl, quinolin-2-yl, indol-5-yl and 1,3-benzodioxol-5-yl.

In another embodiment, R¹ is selected from the group consisting of n-pent-3-yl, cyclopropyl, cyclobutyl, cyclopentyl, 4S-ethylcarbonyl-cyclopent-1S-yl, cyclohexyl, tetrahydropyran-4-yl, piperidin-4-yl, 1-methyl-piperidin-4-yl, 1-ethyl-piperidin-4-yl, 1-isopropyl-piperidin-4-yl, 1-(1-methyl-n-pentyl)-piperidin-4-yl, 1-(n-pentyl)-piperidin-4-yl, 1-(2,2-dimethyl-propyl)-piperidin-4-yl, 1-isobutyl-piperidin-4-yl, 1-propyl-piperidin-4-yl, 1-isopentyl-piperidin-4-yl, 1-(n-hexyl)-piperidin-4-yl, 1-cyclobutyl-piperidin-4-yl, 1-cyclopentyl-piperidin-4-yl, 1-cyclohexyl-piperidin-4-yl, 1-benzyl-piperidin-4-yl, pyrrolidin-3-yl, 1-propyl-pyrrolidin-3-yl, 1-isobutyl-pyrrolidin-3-yl, 1-isopentyl-pyrrolidin-3-yl, 1-(3-methyl-cyclopentyl)-pyrrolidin-3-yl, 1-(cyclopropyl-carbonyl)-pyrrolidin-3-yl, 1-methyl-azetidin-3-yl, 1-(n-butyl)-azetidin-3-yl, 1-isobutyl-azetidin-3-yl, 1-isopentyl-azetidin-3-yl, 1-(2,2-dimethyl-propyl)-azetidin-3-yl, 1-cyclobutyl-azetidin-3-yl, 1-cyclohexyl-azetidin-3-yl, 1-(cyclopropyl-carbonyl)-azetidin-3-yl, phenyl, 2-chloro-phenyl, 3-chloro-phenyl, 4-chloro-phenyl, 2-fluoro-phenyl, 4-dichloro-phenyl, 2,4-dichloro-phenyl, 3,4-dichloro-phenyl, 2,3,4-trifluoro-phenyl, 2,4-difluoro-phenyl, 2-fluoro-4-cyano-phenyl, 2-chloro-4-fluoro-phenyl, 4-isopropyl-phenyl, 3-methoxy-phenyl, 2-methyl-5-fluoro-phenyl, 3-hydroxy-4-methoxy-phenyl, 3-chloro-4-methoxy-phenyl, 4-methoxy-phenyl, 4-methylthio-phenyl, 4-trifluoromethyl-phenyl, 4-cyano-phenyl, thiophen-2-yl, 3-chloro-thiophen-2-yl, 3-5 methyl-thiophen-2-yl, 5-methyl-thiophen-3-yl, thiazol-5-yl, 2-bromo-thiazol-2-yl, pyridin-2-yl, pyridin-4-yl, 2-chloro-pyridin-3-yl, 6-chloro-pyridin-3-yl, 3-fluoro-pyridin-4-yl, 2-chloro-6-methoxy-pyridin-4-yl, 6-methyl-pyridin-4-yl, 6-methoxy-pyridin-3-yl, 5-(dimethylamino)-pyridin-2-yl, 6-(isopropyl-amino)-pyridin-3-yl, 6-(cyclobutyl-amino)-pyridin-3-yl, 6-(piperidin-1-yl)-pyridin-3-yl, 6-(morpholin-4-yl)-pyridin-3-yl, 6-(4-methyl-piperazin-1-yl)-pyridin-3-yl, 6-(N-methyl-N-(1-methyl-piperidin-4-yl)-amino)-pyridin-3-yl, 6-(N-methyl-N-isopropyl-amino)-pyridin-3-yl, 6-(pyrrolidin-1-yl)-pyridin-3-yl, 6-(3S-hydroxymethyl-piperazin-1-yl)-pyridin-3-yl, 6-(3R-hydroxymethyl-piperazin-4-yl)-pyridin-3-yl, 2-chloro-pyrimidin-5-yl, 2-(isopropyl-amino)-pyrimidin-5-yl, 2-(N-methyl-N-isopropyl-amino)-pyrimidin-5-yl, 2-(morpholin-4-yl)-pyrimidin-5-yl, 6-(morpholin-4-yl)-pyrimidin-5-yl, 2-(cyclobutyl-amino)-pyrimidin-5-yl, quinolin-2-yl, indol-5-yl and 1,3-benzodioxol-5-yl.

In another embodiment, R¹ is selected from the group consisting of n-pent-3-yl, cyclopropyl, cyclohexyl, 1-isopropyl-piperidin-4-yl, 1-isobutyl-piperidin-4-yl, 1-cyclopentyl-piperidin-4-yl, 1-cyclohexyl-piperidin-4-yl, 1-methyl-azetidin-3-yl, phenyl, 3-chloro-phenyl, 4-chloro-phenyl, 2-fluoro-phenyl, 2,4-dichloro-phenyl, 2-fluoro-4-cyano-phenyl, 3-methoxy-phenyl, 2-methyl-5-fluoro-phenyl, 3-hydroxy-4-methoxy-phenyl, 4-methoxy-phenyl, 4-methylthio-phenyl, 4-trifluoromethyl-phenyl, 3-chloro-thiophen-2-yl, pyridin-4-yl, 6-chloro-pyridin-3-yl, 3-fluoro-pyridin-4-yl, 6-methyl-pyridin-4-yl, 6-methoxy-pyridin-3-yl, 6-(isopropyl-amino)-pyridin-3-yl, 6-(cyclobutyl-amino)-pyridin-3-yl, 6-(piperidin-1-yl)-pyridin-3-yl, 6-(morpholin-4-yl)-pyridin-3-yl, 6-(4-methyl-piperazin-1-yl)-pyridin-3-yl, 6-(N-methyl-N-(1-methyl-piperidin-4-yl)-amino)-pyridin-3-yl, 6-(N-methyl-N-isopropyl-amino)-pyridin-3-yl, 6-(pyrrolidin-1-yl)-pyridin-3-yl, 6-(3S-hydroxymethyl-piperazin-1-yl)-pyridin-3-yl, 6-(3R-hydroxymethyl-piperazin-4-yl)-pyridin-3-yl, 2-chloro-pyrimidin-5-yl, 2-(isopropyl-amino)-pyrimidin-5-yl, 2-(N-methyl-N-isopropyl-amino)-pyrimidin-5-yl, 2-(morpholin-4-yl)-pyrimidin-5-yl, 6-(morpholin-4-yl)-pyrimidin-5-yl and 2-(cyclobutyl-amino)-pyrimidin-5-yl.

In another embodiment, R¹ is selected from the group consisting of 1-methyl-5 azetidin-3-yl, 1-(n-butyl)-piperidin-4-yl, 1-(2,2-dimethyl-propyl)-piperidin-4-yl, 1-isopentyl-piperidin-4-yl, 1-cyclobutyl-piperidin-4-yl, 1-cyclopentyl-piperidin-4-yl, 1-cyclohexyl-piperidin-4-yl, 4-methylthio-phenyl, 2-fluoro-4-cyano-phenyl, 3-fluoro-pyridin-4-yl, 6-(3S-hydroxymethyl-piperidin-1-yl)-pyridin-3-yl, 6-(isopropyl-amino)-pyridin-3-yl, 6-(cyclobutyl-amino)-pyridin-3-yl, 6-(N-methyl-N-isopropyl-amino)-pyridin-3-yl, 6-(N-methyl-N-(1-methyl-piperidin-4-yl)-amino)-pyridin-3-yl, 6-(morpholin-4-yl)-pyridin-3-yl, 6-(4-methyl-piperazin-1-yl)-pyridin-3-yl, 2-(isopropyl-amino)-pyrimidin-5-yl, 2-(morpholin-4-yl)-pyrimidin-5-yl, 2-(cyclobutyl-amino)-pyrimidin-5-yl and indol-5-yl.

In another embodiment, R¹ is selected from the group consisting of cyclopropyl, 6-chloro-pyridin-3-yl, 6-(isopropyl-amino)-pyridin-3-yl, 6-(N-methyl-N-isopropyl-amino)-pyridin-3-yl and 6-(morpholin-4-yl)-pyridin-3-yl. In another embodiment, R¹ is selected from the group consisting of 6-chloro-pyridin-3-yl and 6-(isopropylamino)-pyridin-3-yl. In an embodiment, R¹ is other than C₁₋₆alkyl or fluorinated C₁₋₃alkyl. In another embodiment, R¹ is other than C₁₋₆alkyl. In an embodiment, R² is selected from the group consisting of halogen, hydroxy, cyano, C₁₋₄alkyl, fluorinated C₁₋₂alkyl, C₁₋₄alkoxy, benzyloxy and —NR^{X}R^{Y}; wherein R^{X} is selected from the group consisting of hydrogen, C₁₋₄alkyl and —(C₂₋₄alkyl)-O—(C₁₋₂alkyl); and wherein R^{Y} is selected from the group consisting of hydrogen, C₁₋₄alkyl, —(C₂₋₄alkyl)-O—(C₁₋₂alkyl), C₃₋₆cycloalkyl and —C(O)—C₃₋₆cycloalkyl. In another embodiment, R² is selected from the group consisting of halogen, hydroxy, C₁₋₂alkyl, C₁₋₂alkoxy, benzyloxy and —NR^{X}R^{Y}; wherein R^{x} is selected from the group consisting of hydrogen, C₁₋₃ alkyl and -(C₂alkyl)-O—(C₁₋₂alkyl); and wherein R^{Y} is selected from the group consisting of hydrogen, C₁₋₃ alkyl, -(C₂alkyl)-O—(C₁₋₂alkyl), C₃cycloalkyl and —C(O)—C₃cycloalkyl. In another embodiment, R² is selected from the group consisting of chloro, hydroxy, methyl, ethyl, methoxy, amino, methyl-amino, isopropyl-amino, (methoxyethyl)-amino, cyclopropyl-amino, (cyclopropylcarbonyl)-amino, N,N-dimethylamino, N-methyl-N-isopropyl-amino, N-methyl-N-(methoxyethyl)-10 amino, N-methyl-N-cyclopropyl-amino, N-(methoxyethyl)-N-(cyclopropylcarbonyl)-amino and benzyloxy. In another embodiment, R² is selected from the group consisting of chloro, hydroxy, methyl, ethyl, methoxy, benzyloxy, methylamino, (methoxyethyl)amino, dimethylamino and N-methyl-N-cyclopropyl-amino. In another embodiment, R² is selected from the group consisting of chloro, methyl, ethyl and methoxy. In another embodiment, R² is methyl. In another embodiment, R² is selected from the group consisting of methyl, amino, methylamino, isopropylamino, (methoxyethyl)amino, cyclopropylamino, dimethylamino and N-methyl-N-cyclopropyl-amino. In an embodiment, R³ is selected from the group consisting of hydrogen, fluoro, chloro, bromo, methyl and trifluoromethyl. In another embodiment, R³ is selected from the group consisting of hydrogen, methyl and trifluoromethyl. In another embodiment, R³ is hydrogen. In an embodiment, m is 0. In another embodiment, m is 1. In an embodiment, n is 0. In another embodiment, n is 1. In an embodiment, m is 0 and n is 0. In an embodiment, m is 1 and n is 1. In an embodiment, m is 1 and n is 0 or alternatively, m is 0 and n is 1.

In some embodiments,

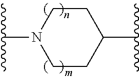

is selected from the group consisting of azetidin-1,3-diyl, pyrrolidin-1,3-diyl, and piperidin-1,4-diyl. In another embodiment,

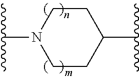

is selected from the group consisting of azetidin-1,3-diyl and piperidin-1,4-diyl. In some embodiments,

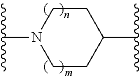

is azetidin-1,3-diyl. In some embodiments,

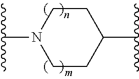

is piperidin-1,4-diyl. In an embodiment, R⁴ is selected from the group consisting of hydrogen and C₁₋₃alkyl. In another embodiment, R⁴ is selected from the group consisting of hydrogen and C₁₋₂alkyl. In another embodiment, R⁴ is selected from the group consisting of hydrogen and methyl. In another embodiment, R⁴ is hydrogen. In an embodiment, R⁵ is selected from the group consisting of hydrogen, hydroxy and C₁₋₃alkyl. In another embodiment, R⁵ is selected from the group consisting of hydrogen, hydroxy and C₁₋₂alkyl. In another embodiment, R⁵ is selected from the group consisting of hydrogen, hydroxy, trans-hydroxy, methyl, trans-methyl and cis-methyl. In another embodiment, R⁵ is selected from the group consisting of hydrogen, methyl and trans-methyl. In another embodiment, the present invention is directed to compounds of formula (XXVII) wherein R⁵ is selected from the group consisting of hydrogen and trans-methyl. In another embodiment, R⁵ is hydrogen. In some embodiments,

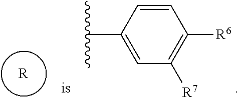

In another embodiment,

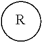

is selected from the group consisting of

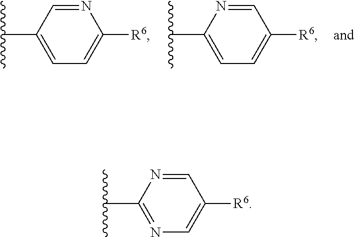

In some embodiments, is

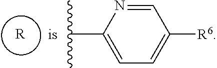

In some embodiments,

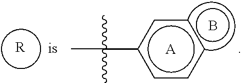

In an embodiment, R⁶ is selected from the group consisting of aryl, 5 to 6 membered heteroaryl and 9 to 10 membered heteroaryl; wherein the aryl, 5 to 6 membered heteroaryl or 9 to 10 membered heteroaryl is optionally substituted with one to two substituents independently selected from the group consisting of halogen, C₁₋₄alkyl, trifluoromethyl, hydroxy substituted C₁₋₂alkyl, C₁₋₄alkoxy, NR^{P}R^{Q}, —(C₁₋₂ alkyl)-NR^{P}R^{Q}, C₃₋₆cycloalkyl, —(C₁₋₂alkyl)-C₃₋₆ cycloalkyl, 5 to 6 membered saturated, nitrogen containing heterocyclyl and 5 to 6 membered nitrogen containing heteroaryl; wherein R^{P} and R^{Q} are each independently selected from the group consisting of hydrogen and C₁₋₄alkyl. In another embodiment, R⁶ is selected from the group consisting of phenyl, 5 to 6 membered heteroaryl and 9 to 10 membered, nitrogen containing heteroaryl; wherein the phenyl, 5 to 6 membered heteroaryl or 9 to 10 membered, nitrogen containing heteroaryl is optionally substituted with a substituent selected from the group consisting of halogen, C₁₋₄alkyl, —(C₁₋₂alkyl)-OH, C₁₋₂alkoxy, NR^{P}R^{Q}, —(C₁₋₂alkyl)-NR^{P}R^{Q}, C₃₋₄cycloalkyl, —(C₁₋₂alkyl)-C₃₋₄cycloalkyl, 6 membered saturated, nitrogen containing heterocyclyl and 6 membered, nitrogen containing heteroaryl; wherein R^{P} and R^{Q} are each independently selected from the group consisting of hydrogen and C₁₋₂alkyl. In another embodiment, R⁶ is selected from the group consisting of phenyl, 2-fluoro-phenyl, 3-fluoro-phenyl, 4-fluoro-phenyl, 2-methyl-phenyl, 3-methyl-phenyl, 4-methyl-phenyl, 2-methoxy-phenyl, 3-methoxy-phenyl, 4-methoxy-phenyl, thiophen-2-yl, thiophen-3-yl, furan-2-yl, furan-3-yl, isoxazol-4-yl, pyridin-3-yl, pyridin-4-yl, 2-amino-pyridin-3-yl, 3-amino-pyridin-4-yl, pyrazol-4-yl, 1-methyl-pyrazol-4-yl, 1-methyl-pyrazol-5-yl, 1-(tetrahydropyran-4-yl)-pyrazol-4-yl, 1-(cyclobutyl-methyl)-pyrazol-4-yl, 1,3-dimethyl-pyrazol-4-yl, 1-isopropyl-pyrazol-4-yl, 1-(2-hydroxyethyl)-pyrazol-4-yl, 1-cyclobutyl-pyrazol-4-yl, 1-(cyclopropyl)-pyrazol-4-yl, 1-(cyclopropyl-methyl)-pyrazol-4-yl, 1-(dimethylamino-ethyl)-pyrazol-4-yl, 1-(pyridin-3-yl)-pyrazol-4-yl, 1-(pyridin-4-yl)-pyrazol-4-yl, 1-methyl-indazol-6-yl, imidazol-1-yl, quinolin-4-yl, quinolin-5-yl and isoquinolin-6-yl. In another embodiment, R⁶ is selected from the group consisting of furan-3-yl, thiophen-3-yl, pyridin-3-yl, pyridin-4-yl, 2-amino-pyridin-3-yl, 3-amino-pyridin-4-yl, imidazol-1-yl, isoxazol-4-yl, pyrazol-4-yl, 1-methyl-pyrazol-4-yl, 1-isopropyl-pyrazol-4-yl, 1-(2-hydroxyethyl)-pyrazol-4-yl, 1-cyclopropyl-pyrazol-4-yl, 1-cyclobutyl-pyrazol-4-yl, 1-(cyclopropyl-methyl)-pyrazol-4-yl, 1,3-dimethyl-pyrazol-4-yl, 1-(pyridin-3-yl)-pyrazol-4-yl, 1-(pyridin-4-yl)-pyrazol-4-yl, 1-methyl-pyrazol-5-yl, quinolin-4-yl, quinolin-5-yl, isoquinolin-6-yl and 1-methyl-indazol-6-yl. In another embodiment, R⁶ is selected from the group consisting of pyridin-4-yl, 2-amino-pyridin-3-yl, 3-amino-pyridin-4-yl, imidazol-1-yl, isoxazol-4-yl, pyrazol-4-yl, 1-methyl-pyrazol-4-yl, 1-(pyridin-4-yl)-pyrazol-4-yl, 1-methyl-pyrazol-5-yl and quinolin-4-yl. In another embodiment, R⁶ is selected from the group consisting of pyridin-4-yl, 3-amino-pyridin-4-yl, 1-methyl-pyrazol-4-yl, 1-(2-hydroxyethyl)-pyrazol-4-yl, 1-cyclopropyl-pyrazol-4-yl, 1-methyl-pyrazol-5-yl and quinolin-4-yl. In another embodiment, R⁶ is 1-methyl-pyrazol-4-yl. In an embodiment, R⁷ is selected from the group consisting of hydrogen, fluoro, chloro, bromo, C₁₋₄alkyl and trifluoromethyl. In another embodiment, R⁷ is selected from the group consisting of hydrogen, halogen, C₁₋₂alkyl and trifluoromethyl. In another embodiment, R⁷ is selected from the group consisting of hydrogen, methyl and trifluoromethyl. In another embodiment, R⁷ is hydrogen.

In some embodiments,

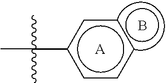

represents a 9 to 10 membered bicyclic, partially unsaturated or aromatic heterocyclyl; and wherein the

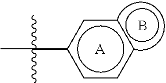

is optionally substituted with one to two substituents independently selected from the group consisting of halogen, oxo, cyano, C₁₋₄alkyl, trifluoromethyl, C₁₋₄akloxy, NR^{S}R^{T} and cyclopropyl; wherein R^{S} and R^{T} are each independently selected from the group consisting of hydrogen and C₁₋₄alkyl. In another embodiment,

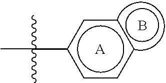

represents a 9 to 10 membered bicyclic, partially unsaturated or aromatic, nitrogen containing heterocyclyl; wherein the

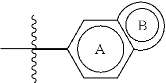

is optionally substituted with one to two substituents independently selected from the group consisting of oxo and C₁₋₂ alkyl. In another embodiment,

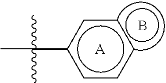

is selected from the group consisting of benzothiazol-6-yl, 2-oxo-benzothiazol-6-yl, 2-oxo-2,3,4-trihydro-quinolin-7-yl, isoquinolin-6-yl, isoquinolin-7-yl, 2-oxo-indolin-5-yl, 1-methyl-2-oxo-isoindol-5-yl, 1,7-dimethyl-isoindol-5-yl, 1-methyl-indazol-6-yl, imidazo[1,2-a]pyridine-6-yl and [1,2,4]triazolo[4,3-a]pyridine-6-yl.

In an embodiment, the compound is selected from the group consisting of 6-(isopropylamino)-N-(2-methyl-5-(4-(4-(1-methyl-1H-pyrazol-4-yl)phenyl)piperidine-1-carbonyl)phenyl)nicotinamide; N-(2-methyl-5-(3-(4-(1-methyl-1H-pyrazol-4-yl)phenyl)azetidine-1-carbonyl)phenyl)-6-morpholinonicotinamide; N-(2-chloro-5-(3-(4-(pyridin-3-yl)phenyl)azetidine-1-carbonyl)phenyl)-6-(isopropylamino)nicotinamide; N-(2-chloro-5-(3-(4-(pyridin-4-yl)phenyl)azetidine-1-carbonyl)phenyl)-6-(isopropylamino)nicotinamide; 6-(isopropyl(methyl)amino)-N-(2-methyl-5-(3-(4-(1-methyl-1H-pyrazol-4-yl)phenyl)azetidine-1-carbonyl)phenyl)nicotinamide; 4-methoxy-N-(2-methyl-5-(3-(4-(1-methyl-1H-pyrazol-4-yl)phenyl)azetidine-1-carbonyl)phenyl)benzamide; N-(2-methyl-5-(4-(4-(1-methyl-1H-pyrazol-4-yl)phenyl)piperidine-1-carbonyl)phenyl)-6-morpholinonicotinamide; 4-chloro-N-(2-methyl-5-(4-(4-(1-methyl-1H-pyrazol-4-yl)phenyl)piperidine-1-carbonyl)phenyl)benzamide; N-(2-Methyl-5-(4-(4-(1-methyl-1H-pyrazol-4-yl)phenyl)piperidine-1-carbonyl)phenyl)-6-(4-methylpiperazin-1-yl)nicotinamide; 6-(isopropylamino)-N-(2-methoxy-5-(4-(4-(1-methyl-1H-pyrazol-4-yl)phenyl)piperidine-1-carbonyl)phenyl)nicotinamide; N-(2-ethyl-5-(4-(4-(1-methyl-1H-pyrazol-4-yl)phenyl)piperidine-1-carbonyl)phenyl)-6-(isopropylamino)nicotinamide; 6-(isopropylamino)-N-(5-(4-(4-(1-methyl-1H-pyrazol-4-yl)phenyl)piperidine-1-carbonyl)-2-(methylamino)phenyl)nicotinamide; and stereoisomers, tautomers and pharmaceutically acceptable salts thereof. In another embodiment, the compound is selected from the group consisting of 6-(isopropylamino)-N-(2-methyl-5-(4-(4-(1-methyl-1H-pyrazol-4-yl)phenyl)piperidine-1-carbonyl)phenyl) nicotinamide; N-(2-methyl-5-(3-(4-(1-methyl-1H-pyrazol-4-yl)phenyl)azetidine-1-carbonyl)phenyl)-6-morpholinonicotinamide; 6-(isopropyl(methyl)amino)-N-(2-methyl-5-(3-(4-(1-methyl-1H-pyrazol-4-yl)phenyl)azetidine-1-carbonyl)phenyl)nicotinamide; N-(2-methyl-5-(4-(4-(1-methyl-1H-pyrazol-4-yl)phenyl)piperidine-1-carbonyl)phenyl)-6-morpholinonicotinamide; N-(2-ethyl-5-(4-(4-(1-methyl-1H-pyrazol-4-yl)phenyl)piperidine-1-carbonyl)phenyl)-6-(isopropylamino)nicotinamide; and stereoisomers, tautomers and pharmaceutically acceptable salts thereof.

In some embodiments, wherein when n is 0 and m is 0, such that

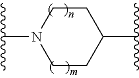

is azetidin-1,3-diyl, then R⁴ is hydrogen and R⁵ is hydrogen. In another embodiment, wherein when n is 1 and m is 1, such that

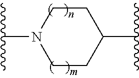

is pyrrolidin-1,3-diyl, then R⁴ is hydrogen and R⁵ is hydrogen. In some embodiments, R¹ is other than C₁₋₂ alkyl. In another embodiment, R¹ is other than C₁₋₄ alkyl. In some embodiments,

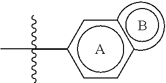

is other than optionally substituted pyrazolo[1,5-a]pyrimidinyl. In some embodiments,

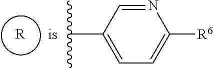

and R⁶ is other than optionally substituted aryl. In another embodiment,

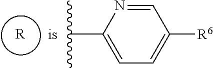

and R⁶ is other than optionally substituted aryl. In some embodiments,

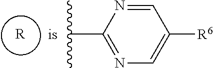

and R⁶ is other than optionally substituted aryl.

In some embodiments, the compound has the structure of Formula (XXVIII):

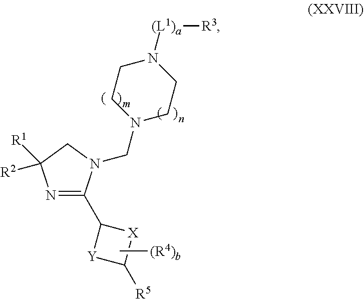

wherein R¹ and R² are taken together with the carbon atom to which they are bound to form an optionally substituted ring structure selected from the group consisting of (a) C₃₋₈cycloalkyl; wherein the C₃₋₈ cycloalkyl is optionally substituted with one to two R¹¹ groups; (b) benzo-fused C₅₋₆cycloalkyl; wherein the benzo-fused C₅₋₆cycloalkyl is bound through a carbon atom of the C₅₋₆cycloalkyl portion of the ring structure; wherein the benzo-fused C₅₋₆cycloalkyl is optionally substituted with one to two R¹¹ groups; and (c) 4 to 8 membered, saturated heterocyclyl; wherein the 4 to 8 membered, saturated heterocyclyl contains one heteroatom selected from the group consisting of O, S and N; wherein the S is optionally substituted with one to two oxo; wherein the N is substituted with R¹⁰; provided that the heteroatom is not present at the 2-position relative to the carbon atom of the imidazolin-5-one; and wherein the 4- to 8-membered, saturated heterocyclyl is optionally substituted with one R¹¹ group, and further optionally substituted with one R¹² group; wherein R¹⁰ is selected from the group consisting of hydrogen, C₁₋₄alkyl, fluorinated C₁₋₄alkyl, —CH₂-(hydroxy substituted C₁₋₄alkyl), —(C₂₋₄alkyl)-O—(C₁₋₄alkyl), —(C₂₋₄alkenyl), —(C₁₋₄ alkyl)-phenyl, —C(O)—NR^{A}R^{B}, —C(O)—(C₁₋₃alkyl)-NR^{A}R^{B}, —C(O)—(C₁₋₄alkyl), —C(O)-(fluorinated C₁₋₂alkyl), —C(O)—(C₃₋₆cycloalkyl), C(O)-phenyl, —C(O)-(5 to 6 membered heteroaryl),

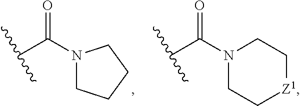

—C(O)O—(C₁₋₄alkyl), —SO₂—(C₁₋₄alkyl), —SO₂—NR^{A}R^{B}, phenyl and 5 to 6 membered heteroaryl; wherein Z¹ is selected from the group consisting of —CH₂—, —O—, —NR_{c}—, —S—, —S(O)— and —SO₂—; wherein R^{A}, R^{B} and R^{C} are each independently selected from the group consisting of hydrogen and C₁₋₄alkyl; and wherein the phenyl or 5 to 6 membered heteroaryl whether alone or as part of a substituent group, is further optionally substituted with one to two substituents independently selected from the group consisting of halogen, hydroxy, cyano, NR^{A}R^{B}, C₁₋₄alkyl, fluorinated C₁₋₄alkyl, C₁₋₄alkoxy and fluorinated C₁₋₄alkoxy; wherein each R¹¹ is independently selected from the group consisting of hydroxy, oxo, halogen, C₁₋₄alkyl, fluorinated C₁₋₄alkyl, C₁₋₄alkoxy, fluorinated C₁₋₄alkoxy, hydroxy substituted C₁₋₄alkyl, —(C₁₋₄alkyl)-O—(C₁₋₄alkyl), —(C₁₋₄alkyl)-phenyl, -cyano, —NR^{D}R^{E}, —C(O)—NR^{D}R^{E}, —C(O)—(C₁₋₄alkyl), —C(O)-phenyl, —C(O)-(5 to 6 membered heteroaryl),

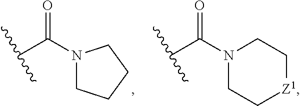

—C(O)OH, —C(O)O—(C₁₋₄alkyl), —SO₂—(C₁₋₄alkyl), —SO₂—NR^{D}R^{E}, phenyl and 5 to 6 membered heteroaryl; wherein Z² is selected from the group consisting of —CH₂—, —O—, —NR_{c}—, —S—, —S(O)— and —SO₂—; wherein R^{D}, R^{E} and R^{F} are each independently selected from the group consisting of hydrogen and C₁₋₄alkyl; and wherein the phenyl or 5 to 6 membered heteroaryl, whether alone or as part of a substituent group, is further optionally substituted with one to two substituents independently selected from the group consisting of halogen, hydroxy, cyano, NR^{D}R^{E}, C₁₋₄alkyl, fluorinated C₁₋₄alkyl, C₁₋₄alkoxy and fluorinated C₁₋₄alkoxy; and wherein R¹² is selected from the group consisting of hydroxy, oxo, halogen, C₁₋₄alkyl, fluorinated C₁₋₄alkyl, C₁₋₄alkoxy, fluorinated C₁₋₄alkoxy and hydroxy substituted C₁₋₄alkyl; m is an integer from 0 to 1; and n is an integer from 0 to 2; provided that when n is 2 then m is 1;

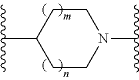

is selected from the group consisting of azetidin-3-yl, pyrrolidin-3-yl, pyrrolidin-3R-yl, pyrrolidin-3S-yl, piperidin-3-yl, piperidin-3R-yl, piperidin-3S-yl, and piperidin-4-yl; a is an integer from 0 to 1; L¹ is selected from the group consisting of —C(O)—, —C(O)O—, —C(O)—NR^{L}—, —C(S)—, —SO₂—, —SO₂—NR^{L}—; wherein R^{L} is selected from the group consisting of hydrogen and C₁₋₄alkyl; R³ is selected from the group consisting of C₁₋₄alkyl, fluorinated C₁₋₄alkyl, hydroxy substituted C₁₋₄alkyl, C₂₋₄alkenyl, C₃₋₆cycloalkyl, —(C₁₋₄alkyl)-(C₃₋₆cycloalkyl), 4 to 6 membered saturated heterocyclyl, —(C₁₋₄alkyl)-(4 to 6 membered, saturated heterocyclyl), —(C₂₋₄alkenyl)-(5 to 6 membered saturated heterocyclyl), 5 to 6 membered heteroaryl, —(C₁₋₄alkyl)-(5 to 6 membered heteroaryl), —(C₂₋₄alkenyl)-(5 to 6 membered heteroaryl), and NR^{V}R^{W}; wherein R^{V} and R^{W} are each independently selected from the group consisting of hydrogen and C₁₋₄alkyl; wherein the C₃₋₆cycloalkyl, 4 to 6 membered saturated heterocyclyl or 5 to 6 membered heteroaryl, whether alone or as part of a substituent group, is optionally substituted with one to two substituents independently selected from the group consisting of halogen, hydroxy, cyano, C₁₋₄alkyl, fluorinated C₁₋₄alkyl, —(C₁₋₄alkyl)-OH, C₁₋₄alkoxy, fluorinated C₁₋₄alkoxy, and NR^{G}R^{H}; wherein R^{G} and R^{H} are each independently selected from the group consisting of hydrogen and C₁₋₄alkyl;

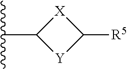

is selected from the group consisting of

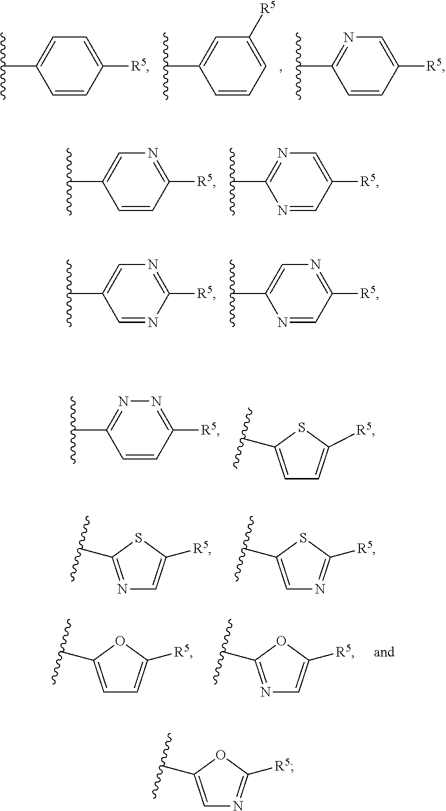

b is an integer from 0 to 2; each R⁴ is independently selected from the group consisting of hydroxy, halogen, C₁₋₄alkyl, fluorinated C₁₋₄alkyl, C₁₋₄alkoxy, fluorinated C₁₋₄alkoxy, cyano, and NR^{J}R^{K}, wherein R^{J} and R^{K} are each independently selected from the group consisting of hydrogen and C₁₋₄alkyl; provided that each R⁴ group is bound to a carbon atom; provided that when

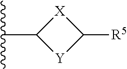

is selected from the group consisting of

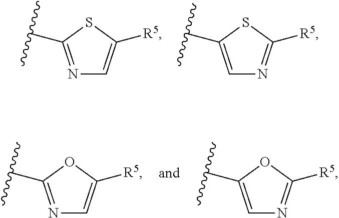

and substituted with —(R⁴)_{b}, then b is an integer from 0 to 1; R⁵ is selected from the group consisting of (a)

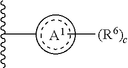

and (b)

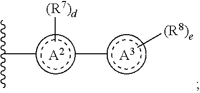

wherein

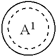

is selected from the group consisting of aryl, heteroaryl, and partially unsaturated heterocyclyl; c is an integer from 0 to 2; each R⁶ is independently selected from the group consisting of hydroxy, oxo, halogen, cyano, nitro, C₁₋₄alkyl, fluorinated C₁₋₄alkyl, hydroxy substituted C₁₋₄alkyl, —(C₁₋₄alkyl)-CN, —(C₁₋₄alkyl)-O—(C₁₋₄alkyl), C₁₋₄alkoxy, fluorinated C₁₋₄alkoxy, —SO₂—(C₁₋₄alkyl), —NR^{M}R^{N}, —(C₁₋₄alkyl)-NR^{P}R^{Q}, —C(O)—(C₁₋₄alkyl), —C(O)-(fluorinated C₁₋₂alkyl), —C(O)—NR^{M}R^{N}, —C(O)OH, —C(O)O—(C₁₋₄alkyl), —NR^{M}—C(O)H, —NR^{M}—C(O)—(C₁₋₄alkyl), —NR^{M}—SO₂—(C₁₋₄alkyl), C₃₋₆cycloalkyl, -cyano-(C₃₋₆cycloalkyl), —(C₁₋₄alkyl)-(C₃₋₆cycloalkyl), —S—(C₃₋₆ cycloalkyl), —SO—(C₃₋₆cycloalkyl), —SO₂—(C₃₋₆ cycloalkyl), —NH—(C₃₋₆cycloalkyl), —NH—SO₂—(C₃₋₆cyclalkyl), oxetanyl, —(C₁₋₂alkyl)-oxetanyl, tetrahydrofuranyl, —(C₁₋₂alkyl)-tetrahydro-furanyl, tetrahydro-pyranyl, and —(C₁₋₂alkyl)-tetrahydro-pyranyl; wherein R^{M} and R^{N} are each independently selected from the group consisting of hydrogen and C₁₋₄alkyl; wherein R^{P} and R^{Q}are each independently selected from the group consisting of hydrogen and C₁₋₄alkyl; alternatively R^{P} and R^{Q} are taken together with the nitrogen atom to which they are bound to form a 5 to 6 membered saturated heterocyclyl; such 5 to 6 membered saturated heterocyclyl is optionally substituted with a substituent selected from the group consisting of halogen, C₁₋₄alkyl and fluorinated C₁₋₄alkyl; wherein

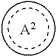

is selected from the group consisting of phenyl and 5 to 6 membered heteroaryl; d is an integer from 0 to 1; R⁷ is selected from the group consisting of hydroxy, halogen, cyano, nitro, C₁₋₄alkyl, fluorinated C₁₋₄alkyl, hydroxy substituted C₁₋₄alkyl, C₁₋₄alkoxy, fluorinated C₁₋₄alkoxy, —NR^{R}R^{S}, —C(O)—NR^{R}R^{S}, —C(O)OH and —C(O)O—(C₁₋₄alkyl); wherein R^{R} and R^{S} are each independently selected from the group consisting of hydrogen and C₁₋₄alkyl; wherein

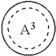

is selected from the group consisting of phenyl, 5 to 6 membered saturated heterocyclyl and 5 to 6 membered heteroaryl; e is an integer from 0 to 2; each R⁸ is independently selected from the group consisting of hydroxy, halogen, cyano, nitro, C₁₋₄alkyl, fluorinated C₁₋₄alkyl, hydroxy substituted C₁₋₄alkyl, C₁₋₄alkoxy, fluorinated C₁₋₄alkoxy, —NR^{T}R^{U}, —C(O)—NR^{T}R^{U}, —C(O)OH, —C(O)O—(C₁₋₄alkyl), —(C₁₋₄alkyl)-NR^{T}R^{U}, C₃₋₅cycloalkyl, —(C₁₋₂alkyl)-(C₃₋₅cycloalkyl), oxetanyl, —(C₁₋₂alkyl)-oxetanyl, tetrahydrofuranyl, —(C₁₋₂alkyl)-tetrahydrofuranyl, tetrahydropyranyl, and —(C₁₋₂alkyl)-tetrahydropyranyl; wherein R^{T} and R^{U} are each independently selected from the group consisting of hydrogen and C₁₋₄alkyl; provided that when

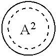

is a 5-membered heteroaryl, then

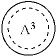

is bound at the 3-position, relative to the point of attachment of the

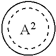

to the

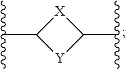

provided further that when

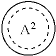

is phenyl or a 6 membered heteroaryl, then

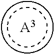

is bound at the 3- or 4-position, relative to the point of attachment of the

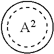

to the

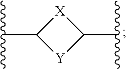

provided that when R¹ and R² are taken together with the carbon atom to which they are bound to form 1-(methoxycarbonyl)-azetidin-3-yl, m is 1 and n is 0 or m is 0 and n is 1;

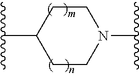

is pyrrolidin-3R-yl; -(L¹)ₐ-R³ is selected from the group consisting of —C(O)—CF₃, —C(O)-cyclopropyl, —C(O)-(thiazol-2-yl), —C(O)OCH₃, or —SO₂—CH₃,

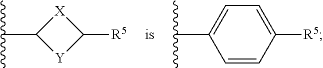

and b is 0; then R⁵ is other than quinolin-7-yl, benzofuran-5-yl, 1-methyl-indazol-5-yl, 1-methyl-pyrazol-4-yl, 4-(1-methyl-pyrazol-4-yl)-phenyl, 1,2,3,4,4a,8a-hexahydro-2-methyl-carbonyl-isoquinolin-6-yl) and 1,2,3,4-trihydro-2-methylcarbonyl-isoquinolin-2-yl; provided further that when R¹ and R² are taken together with the carbon atom to which they are bound to form cyclopentyl; m is 1 and n is 0 or m is 0 and n is 1;

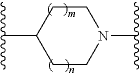

is pyrrolidin-3R-yl; -(L¹)ₐ-R³ is —C(O)-cyclopropyl;

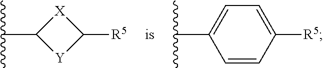

b is 0 or (R⁴)_{b} is 2-methyl; then R⁵ is other than 1-methyl-pyrazol-4-yl, 4-methyl-3,4-dihydro-pyrido[2,3-b]oxazon-7-yl, 2-(piperazin-1-yl)-pyridin-4-yl or 2-(4-methyl-piperazin-1-yl)-pyridin-4-yl; provided further that when R¹ and R² are taken together with the carbon atom to which they are bound to form cyclopentyl; m is 1 and n is 0 or m is 0 and n is 1;

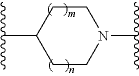

is pyrrolidin-3R-yl; -(L¹)ₐ-R³ is —SO₂-pyrrolidin-1-yl;

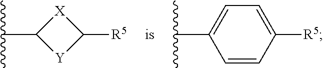

b is 0 or (R⁴)_{b} is 2-methyl; then R⁵ is other than benzofuran-5-yl; provided further that when R¹ and R² are taken together with the carbon atom to which they are bound to form cyclopropyl; m is 0, n is 0,

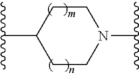

is azetidin-3-yl; -(L¹)ₐ-R³ is selected from the group consisting of —C(O)-cyclopropyl, —C(O)-(1-methyl-cyclopropyl) and —C(O)-(1-hydroxy-cyclopropyl);

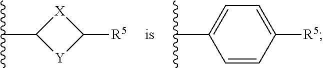

b is 0 or (R⁴)_{b} is selected from the group consisting of 2-fluoro and 2-methyl; then R⁵ is other than 1-isopropylsulfonyl-phenyl, 1-methyl-indazol-5-yl, 1-isopropyl-indazol-5-yl, 1-oxetan-3-yl, indazol-5-yl, 1-methyl-pyrazol-4-yl, 4-methyl-7-bromo-quinolin-2-yl, 5-(2-hydroxy-2-methyl-propyl)-pyridin-2-yl, 6-isopropyl-pyridin-3-yl, 6-(1-cyanomethyl)-pyridin-3-yl, 6-(2-hydroxy-2-methyl-propyl)-pyridin-3-yl, 1,5-naphthyridin-3-yl, 3-methyl-[1,2,4]triazolo[4,3-a]pyridin-6-yl, 4-(1-isobutyl-pyrazol-5-yl)-phenyl or 6-(morpholin-4-yl)-pyridin-3-yl; provided further than when R₁ and R² are taken together with the carbon atom to which they are bound to form cyclopropyl; m is 0, n is 0,

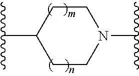

is azetidin-3-yl; -(L¹)ₐ-R³ is —C(O)-(1-hydroxy-cyclopropyl);

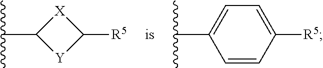

and (R⁴)_{b} is 2-methyl; then R⁵ is other than 1-methyl-indazol-5-yl; provided further that when R¹ and R² are taken together with the carbon atom to which they are bound to form cyclopropyl; m is 0, n is 0,

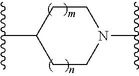

is azetidin-3-yl; -(L¹)ₐ-R³ is —C(O)-pyridin-3-yl;

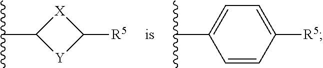

(R⁴)_{b} is 2-methyl, then R⁵ is other than 1-methyl-indazol-5-yl; provided further that when R¹ and R² are taken together with the carbon atom to which they are bound to form cyclopropyl; m is 0, n is 2,

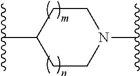

is piperidin-3R-yl or piperidin-3S-yl; -(L¹)ₐ-R³ is —C(O)-cyclopropyl;

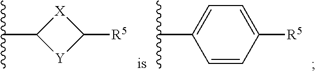

and b is 0, then R⁵ is other than indazol-5-yl, benzofuran-5-yl, benzothien-5-yl, 1-methyl-indazol-5-yl, 4-(4-methylphenyl)phenyl or 4-(3-chlorophenyl)-phenyl; provided further that when R¹ and R² are taken together with the carbon atom to which they are bound to form cyclopropyl, m is 1, n is 1,

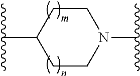

is piperidin-4-yl; -(L¹)ₐ-R³ is —C(O)-cyclopropyl;

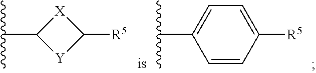

and b is 0, then R⁵ is other than 4-trifluoromethyl-phenyl, 1-methyl-pyrazol-4-yl, benzoxazol-5-yl, pyridine-4-yl or 4-(1-methyl-pyrazol-4-yl)-phenyl; provided further that when R¹ and R² are taken together with the carbon atom to which they are bound to form cyclopropyl; m is 0 and n is 1 or m is 1 and n is 0

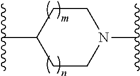

is pyrrolidin-3R-yl; -(L¹)ₐ-R³ is —C(O)-cyclopropyl;

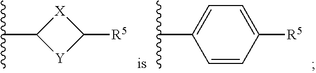

and b is 0, then R⁵ is other than 5-chloro-pyridin-3-yl, 2-oxo-3,4-dihydro-quinolin-7-yl or 6-(4-methyl-piperazin-1-yl)-pyridin-3-yl; provided further that when R¹ and R² are taken together with the carbon atom to which they are bound to form tetrahydrofuran-3,3-diyl or tetrahydropyran-4,4-diyl, m is an integer from 0 to 1 and n is 0 or m is 0 and n is an integer from 0 to 1;

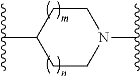

is selected from the group consisting of azetidin-3-yl, pyrrolidin-3R-yl, and pyrrolidin-3-yl; -(L¹)ₐ-R³ is selected from the group consisting of —C(O)-thiazol-2-yl, —C(O)—CF₃, —C(O)OCH₃, and —SO₂—CH₃,

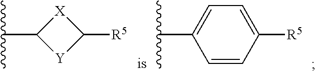

and b is 0, then R⁵ is other than quinolin-7-yl, 1-methyl-indazol-5-yl, benzofuran-5-yl, or 4-(1-methyl-pyrazol-4-yl)-phenyl; and stereoisomers, tautomers, and pharmaceutically acceptable salts thereof.

In some embodiments, the compound has the structure of Formula (XXVIII):

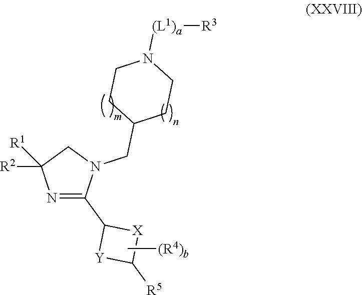

wherein R¹ and R² are taken together with the carbon atom to which they are bound to form an optionally substituted ring structure selected from the group consisting of (a) C₃₋₈cycloalkyl; wherein the C₃₋₈cycloalkyl is optionally substituted with one to two R¹¹ groups; (b) benzo-fused C₅₋₆cycloalkyl; wherein the benzo-fused C₅₋₆cycloalkyl is bound through a carbon atom of the C₅₋₆cycloalkyl portion of the ring structure; wherein the benzo-fused C₅₋₆cycloalkyl is optionally substituted with one to two R¹¹ groups; and (c) 4 to 8 membered, saturated heterocyclyl; wherein the 4 to 8 membered, saturated heterocyclyl contains one heteroatom selected from the group consisting of O, S and N; wherein the S is optionally substituted with one to two oxo; wherein the N is substituted with R¹⁰; provided that the heteroatom is not present at the 2-position relative to the carbon atom of the imidazolin-5-one; and wherein the 4 to 8 membered, saturated heterocyclyl is optionally substituted with one R¹¹ group, and further optionally substituted with one R¹² group; wherein R¹⁰ is selected from the group consisting of hydrogen, C₁₋₄alkyl, fluorinated C₁₋₄alkyl, —CH₂-(hydroxy substituted C₁₋₄alkyl), —(C₂₋₄alkyl)-O—(C₁₋₄alkyl), —(C₂₋₄alkenyl), —(C₁₋₄alkyl)-phenyl, —C(O)—NR^{A}R^{B}, —C(O)—(C₁₋₃alkyl)-NR^{A}R^{B}, —C(O)—(C₁₋₄alkyl), —C(O)-(fluorinated C₁₋₂alkyl), —C(O)—(C₃₋₆cycloalkyl), C(O)-phenyl, —C(O)-(5 to 6 membered heteroaryl),

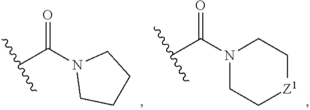

—C(O)O—(C₁₋₄alkyl), —SO₂—(C₁₋₄alkyl), —SO₂—NR^{A}R^{B}, phenyl and 5 to 6 membered heteroaryl; wherein Z¹ is selected from the group consisting of —CH₂—, —O—, —NR_{c}—, —S—, —S(O)— and —SO₂—; wherein R^{A}, R^{B} and R^{C} are each independently selected from the group consisting of hydrogen and C₁₋₄alkyl; and wherein the phenyl or 5 to 6 membered heteroaryl whether alone or as part of a substituent group, is further optionally substituted with one to two substituents independently selected from the group consisting of halogen, hydroxy, cyano, NR^{A}R^{B}, C₁₋₄alkyl, fluorinated C₁₋₄alkyl, C₁₋₄alkoxy and fluorinated C₁₋₄alkoxy; wherein each R¹¹ is independently selected from the group consisting of hydroxy, oxo, halogen, C₁₋₄alkyl, fluorinated C₁₋₄alkyl, C₁₋₄alkoxy, fluorinated C₁₋₄alkoxy, hydroxy substituted C₁₋₄alkyl, —(C₁₋₄alkyl)-O—(C₁₋₄ alkyl), —(C₁₋₄alkyl)-phenyl, -cyano, —NR^{D}R^{E}, —C(O)—NR^{D}R^{E}, —C(O)—(C₁₋₄alkyl), —C(O)-phenyl, —C(O)-(5 to 6 membered heteroaryl),

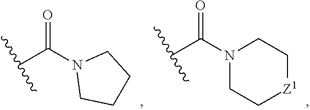

—C(O)OH, —C(O)O—(C₁₋₄alkyl), —SO₂—(C₁₋₄alkyl), —SO₂—NR^{D}R^{E}, phenyl and 5 to 6 membered heteroaryl; wherein Z² is selected from the group consisting of —CH₂—, —O—, —NRₒ—, —S—, —S(O)— and —SO₂—; wherein R^{D}, R^{E} and R^{F} are each independently selected from the group consisting of hydrogen and C₁₋₄alkyl; and wherein the phenyl or 5 to 6 membered heteroaryl, whether alone or as part of a substituent group, is further optionally substituted with one to two substituents independently selected from the group consisting of halogen, hydroxy, cyano, NR^{D}R^{E}, C₁₋₄alkyl, fluorinated C₁₋₄alkyl, C₁₋₄alkoxy and fluorinated C₁₋₄alkoxy; and wherein R¹² is selected from the group consisting of hydroxy, oxo, halogen, C₁₋₄alkyl, fluorinated C₁₋₄alkyl, C₁₋₄alkoxy, fluorinated C₁₋₄alkoxy and hydroxy substituted C₁₋₄alkyl; m is an integer from 0 to 1; and n is an integer from 0 to 2; provided that when n is 2 then m is 1;

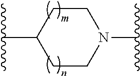

is selected from the group consisting of azetidin-3-yl, pyrrolidin-3-yl, pyrrolidin-3R-yl, pyrrolidin-3S-yl, piperidin-3-yl, piperidin-3R-yl, piperidin-2S-yl, and piperidin-4-yl; a is an integer from 0 to 1; L¹ is selected from the group consisting of —C(O)—, —C(O)O—, —C(O)—NR^{L}—, —C(S)—, —SO₂—, —SO₂—NR^{L}—; wherein R^{L} is selected from the group consisting of hydrogen and C₁₋₄alkyl; R³ is selected from the group consisting of C₁₋₄alkyl, fluorinated C₁₋₄alkyl, hydroxy substituted C₁₋₄alkyl, C₂₋₄alkenyl, C₃₋₆cycloalkyl, —(C₁₋₄alkyl)-(C₃₋₆cycloalkyl), 4 to 6 membered, saturated heterocyclyl, —(C₁₋₄alkyl)-(4 to 6 membered, saturated heterocyclyl), —(C₂₋₄alkenyl)-(5 to 6 membered, saturated heterocyclyl), 5 to 6 membered heteroaryl, —(C₁₋₄alkyl)-(5 to 6 membered heteroaryl), —(C₂₋₄alkenyl)-(5 to 6 membered heteroaryl), and NR^{V}R^{W}; wherein R^{V} and R^{W} are each independently selected from the group consisting of hydrogen and C₁₋₄alkyl; wherein the C₃₋₆cycloalkyl, 4 to 6 membered saturated heterocyclyl or 5 to 6 membered heteroaryl, whether alone or as part of a substituent group, is optionally substituted with one to two substituents independently selected from the group consisting of halogen, hydroxy, cyano, C₁₋₄alkyl, fluorinated C₁₋₄alkyl, —(C₁₋₄alkyl)-OH, C₁₋₄alkoxy, fluorinated C₁₋₄alkoxy, and NR^{G}R^{H}; wherein R^{G} and R^{H} are each independently selected from the group consisting of hydrogen and C₁₋₄alkyl;

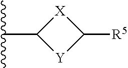

is selected from the group consisting of

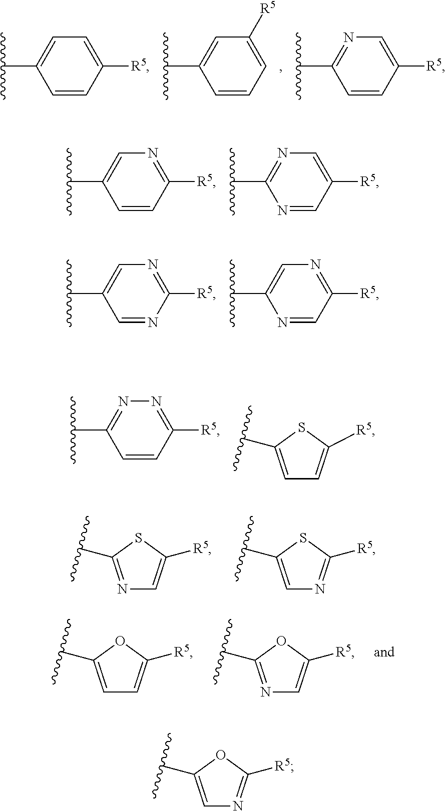

b is an integer from 0 to 2; each R⁴ is independently selected from the group consisting of hydroxy, halogen, C₁₋₄alkyl, fluorinated C₁₋₄alkyl, C₁₋₄alkoxy, fluorinated C₁₋₄alkoxy, cyano, and NR^{J}R^{K}, wherein R^{J} and R^{K} are each independently selected from the group consisting of hydrogen and C₁₋₄alkyl; provided that each R⁴ group is bound to a carbon atom; provided that when

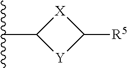

is selected from the group consisting of

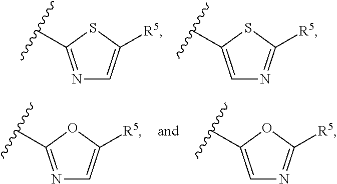

and substituted with —(R⁴)_{b}, then b is an integer from 0 to 1; R⁵ is selected from the group consisting of (a)

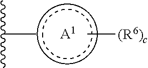

and

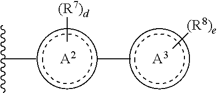

(b); wherein

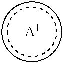

is selected from the group consisting of aryl, heteroaryl, and partially unsaturated heterocyclyl; c is an integer from 0 to 2; each R⁶ is independently selected from the group consisting of hydroxy, halogen, cyano, nitro, C₁₋₄alkyl, fluorinated C₁₋₄alkyl, hydroxy substituted C₁₋₄alkyl, C₁₋₄alkoxy, fluorinated C₁₋₄alkoxy, —NR^{M}R^{N}, —(C₁₋₄alkyl)-NR^{P}R^{Q}, —C(O)—(C₁₋₄alkyl), —C(O)—NR^{M}R^{N}, —C(O)OH, —C(O)O—(C₁₋₄alkyl), —NR^{M}—C(O)H, —NR^{M}—C(O)—(C₁₋₄alkyl), and —NR^{M}—SO₂—(C₁₋₄alkyl); wherein R^{M} and R^{N} are each independently selected from the group consisting of hydrogen and C₁₋₄alkyl; wherein R^{P} and R^{Q} are each independently selected from the group consisting of hydrogen and C₁₋₄alkyl; alternatively R^{P} and R^{Q} are taken together with the nitrogen atom to which they are bound to form a 5 to 6 membered saturated heterocyclyl; such 5 to 6 membered saturated heterocyclyl is optionally substituted with a substituent selected from the group consisting of halogen, C₁₋₄alkyl and fluorinated C₁₋₄alkyl; wherein

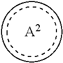

is selected from the group consisting of phenyl and 5 to 6 membered heteroaryl; d is an integer from 0 to 1; R⁷ is selected from the group consisting of hydroxy, halogen, cyano, nitro, C₁₋₄alkyl, fluorinated C₁₋₄alkyl, hydroxy substituted C₁₋₄alkyl, C₁₋₄alkoxy, fluorinated C₁₋₄alkoxy, —NR^{R}R^{S}, —C(O)—NR^{R}R^{S}, —C(O)OH and —C(O)O—(C₁₋₄alkyl); wherein R^{R} and R^{S} are each independently selected from the group consisting of hydrogen and C₁₋₄alkyl; wherein

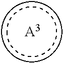

is selected from the group consisting of phenyl, 5 to 6 membered saturated heterocyclyl and 5 to 6 membered heteroaryl; e is an integer from 0 to 2; each R⁸ is independently selected from the group consisting of hydroxy, halogen, cyano, nitro, fluorinated C₁₋₄alkyl, hydroxy substituted C₁₋₄alkyl, C₁₋₄alkoxy, fluorinated C₁₋₄alkoxy, —NR^{T}R^{U}, —C(O)—NR^{T}R^{U}, —C(O)OH, —C(O)O—(C₁₋₄alkyl), and —(C₁₋₄alkyl)-NR^{T}R^{U}; wherein R^{T} and R^{U} are each independently selected from the group consisting of hydrogen and C₁₋₄alkyl; provided that when

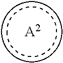

is a 5-membered heteroaryl, then

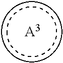

is bound at the 3-position, relative to the point of attachment of the

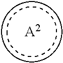

to the

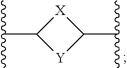

provided further that when

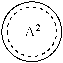

is phenyl or a 6 membered heteroaryl, then

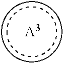

is bound at the 3- or 4-position, relative to the point of attachment of the

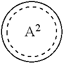

to the

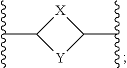

and a stereoisomer, tautomer, and a pharmaceutically acceptable salt thereof.

In some embodiments, R¹ and R² are taken together to form an optionally substituted ring structure selected from the group consisting of (a) C₃₋₆cycloalkyl; wherein the C₃₋₈cycloalkyl is optionally substituted with one R¹¹ group; (b) benzo-fused C₅₋₆cycloalkyl; wherein the benzo-fused C₅₋₆cycloalkyl is bound through a carbon atom of the C₅₋₆cycloalkyl portion of the ring structure; and wherein the benzo-fused C₅₋₆cycloalkyl is optionally substituted with one R¹¹ group; and (c) 4 to 6 membered, saturated heterocyclyl; wherein the 4 to 6 membered, saturated heterocyclyl contains O or NR¹⁰; provided that the O or NR¹⁰ is not present at the 2-position relative to the carbon atom of the imidazolin-5-one; and wherein the 4 to 6 membered, saturated heterocyclyl containing the O or NR¹⁰ is optionally substituted with one R¹¹ group and further optionally substituted with one R¹²; wherein R¹⁰ is selected from the group consisting of hydrogen, C₁₋₄alkyl, fluorinated C₁₋₄alkyl, —CH₂-(hydroxy substituted C₁₋₄alkyl), —(C₂₋₄alkenyl), —(C₁₋₄alkyl)-phenyl, -(C₂alkyl)-O—(C₁₋₄alkyl), —C(O)O—(C₁₋₄alkyl), —C(O)—(C₁₋₄alkyl), —C(O)-(fluorinated C₁₋₂ alkyl), —C(O)—(C₃₋₆cycloalkyl,

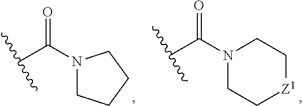

—C(O)NR^{A}R^{B}, —SO₂—(C₁₋₂alkyl); wherein Z¹ is selected from the group consisting of —CH₂—, —O—, —NR_{c}—, —S—, —S(O)— and —SO₂—; and wherein R^{A}, R^{B} and R^{C} are each independently selected from the group consisting of hydrogen and C₁₋₄alkyl; wherein each R¹¹ is independently selected from the group consisting of hydroxy, oxo, halogen, C₁₋₄alkyl, fluorinated C₁₋₄alkyl, C₁₋₄alkoxy, fluorinated C₁₋₄alkoxy, hydroxy substituted C₁₋₄alkyl, —(C₁₋₄alkyl)-phenyl, cyano, —NR^{D}R^{E}, —C(O)—NR^{D}R^{E}, —C(O)—(C₁₋₄alkyl), —C(O)OH, and —C(O)O—(C₁₋₄alkyl); wherein R¹² is selected from the group consisting of hydroxy, oxo, halogen, C₁₋₂alkyl, CF₃, C₁₋₂alkoxy, —OCF₃, and hydroxy substituted C₁₋₂alkyl; m is an integer from 0 to 1; and n is an integer from 0 to 2; provided that when n is 2 then m is 1;

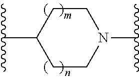

is selected from the group consisting of azetidin-3-yl, pyrrolidin-3-yl, pyrrolidin-3R-yl, pyrrolidin-3S-yl, piperidin-3-yl, piperidin-3R-yl, piperidin-3S-yl, and piperidin-4-yl; a is 1; L¹ is selected from the group consisting of —C(O)—, —C(O)O—, —C(O)—NR^{L}—, and —SO₂—; wherein R^{L} is selected from the group consisting of hydrogen and methyl; R³ is selected from the group consisting of C₁₋₄alkyl, fluorinated C₁₋₂alkyl, hydroxy substituted C₁₋₄alkyl, C₂₋₄alkenyl, C₃₋₆cycloalkyl, 4 to 6 membered saturated heterocyclyl, 5 to 6 membered heteroaryl, and NR^{V}R^{W}; wherein R^{V} and R^{W} are each independently selected from the group consisting of hydrogen and C₁₋₂alkyl; wherein the C₃₋₆cycloalkyl, 4 to 6 membered saturated heterocyclyl or 5 to 6 membered heteroaryl, is optionally substituted with one to two substituents independently selected from the group consisting of halogen, hydroxy, cyano, C₁₋₄alkyl, fluorinated C₁₋₄alkyl, —(C₁₋₂alkyl)-OH, C₁₋₄alkoxy, fluorinated C₁₋₄alkoxy, and NR^{G}R^{H}; wherein R^{G} and R^{H}are each independently selected from the group consisting of hydrogen and C₁₋₄alkyl;

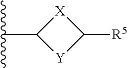

is selected from the group consisting of

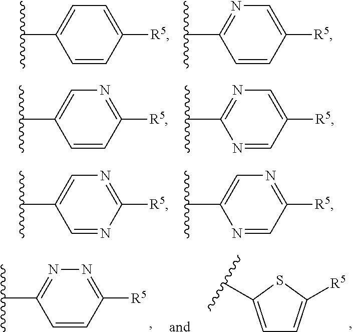

b is an integer from 0 to 1; R⁴ is selected from the group consisting of halogen, C₁₋₄alkyl, fluorinated C₁₋₄alkyl, C₁₋₄alkoxy, fluorinated C₁₋₄alkoxy, and NR^{J}R^{K}, wherein R^{J} and R^{K} are each independently selected from the group consisting of hydrogen and C₁₋₂alkyl; provided that the R⁴ group is bound to a carbon atom; R⁵ is selected from the group consisting of (a)

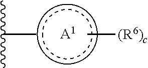

and

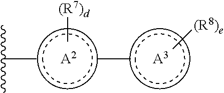

(b); wherein

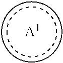

is selected from the group consisting of aryl, heteroaryl, and partially unsaturated heterocyclyl; c is an integer from 0 to 2; each R⁶ is independently selected from the group consisting of hydroxy, oxo, halogen, cyano, nitro, C₁₋₄alkyl, fluorinated C₁₋₄alkyl, hydroxy substituted C₁₋₄alkyl, —(C₁₋₄alkyl)-CN, —(C₁₋₄alkyl)-O—(C₁₋₄alkyl), C₁₋₄alkoxy, fluorinated C₁₋₄alkoxy, —SO₂—(C₁₋₄alkyl), —C(O)—(C₁₋₄alkyl), —C(O)-(fluorinated C₁₋₂alkyl), —C(O)OH, —C(O)O—(C₁₋₄alkyl), —C(O)—NR^{M}R^{N}, —NR^{M}—C(O)H, —NR^{M}R^{N}, —NR^{M}—C(O)—(C₁₋₄alkyl), —NR^{M}—SO₂—(C₁₋₄alkyl), C₃₋₅cycloalkyl, 1-cyano-(C₃₋₅cycloalkyl), —(C₁₋₂alkyl)-(C₃₋₅cycloalkyl), —S—(C₃₋₅cycloalkyl), oxetanyl, and tetrahydrofuranyl, wherein R^{M} and R^{N} are each independently selected from the group consisting of hydrogen and C₁₋₄alkyl; wherein

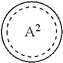

is selected from the group consisting of phenyl and 5 to 6 membered heteroaryl; d is an integer from 0 to 1; R⁷ is selected from the group consisting of hydroxy, halogen, cyano, C₁₋₄alkyl, fluorinated C₁₋₄alkyl, hydroxy substituted C₁₋₄alkyl, C₁₋₄alkoxy, and fluorinated C₁₋₄alkoxy; wherein

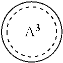

is selected from the group consisting of phenyl, 5 to 6 membered saturated heterocyclyl and 5 to 6 membered heteroaryl; e is an integer from 0 to 2; each R⁸ is independently selected from the group consisting of hydroxy, halogen, cyano, C₁₋₄alkyl, fluorinated C₁₋₄alkyl, hydroxy substituted C₁₋₄alkyl, C₁₋₄alkoxy, fluorinated C₁₋₄alkoxy, —NR^{T}R^{U}, —C(O)—NR^{T}R^{U}, —C(O)OH, —C(O)O—(C₁₋₄alkyl), —(C₁₋₄alkyl)-NR^{T}R^{U}, C₃₋₅cycloalkyl, —(C₁₋₂alkyl)-(C₃₋₅cycloalkyl), oxetanyl, and tetrahydrofuranyl; wherein R^{T} and R^{U} are each independently selected from the group consisting of hydrogen and C₁₋₄alkyl; provided that when

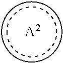

is a 5-membered heteroaryl, then

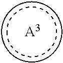

is bound at the 3-position, relative to the point of attachment of the

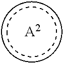

to the

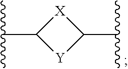

provided further that when

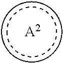

is phenyl or a 6 membered heteroaryl, then

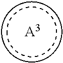

is bound at the 3- or 4-position, relative to the point of attachment of the

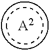

to the

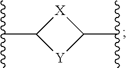

and a stereoisomer, a tautomer, and a pharmaceutically acceptable salt thereof.

In another embodiment, R¹ and R² are taken together to form an optionally substituted ring structure selected from the group consisting of (a) C₃₋₆cycloalkyl; wherein the C₃₋₈cycloalkyl is optionally substituted with one R¹¹ group; (b) benzo-fused C₅₋₆cycloalkyl; wherein the benzo-fused C₅₋₆cycloalkyl is bound through a carbon atom of the C₅₋₆cycloalkyl portion of the ring structure; and wherein the benzo-fused C₅₋₆cycloalkyl is optionally substituted with one R¹¹ group; and (c) 4 to 8 membered, saturated heterocyclyl; wherein the 4 to 8 membered, saturated heterocyclyl contains O or NR¹⁰; provided that the O or NR¹⁰ is not present at the 2-position relative to the carbon atom of the imidazolin-5-one; and wherein the 4 to 8 membered, saturated heterocyclyl containing the O or NR¹⁰ is optionally substituted with one R¹¹ group and further optionally substituted with one R¹²; wherein R¹⁰ is selected from the group consisting of hydrogen, C₁₋₄alkyl, fluorinated C₁₋₄alkyl, —CH₂-(hydroxy substituted C₁₋₄alkyl), —(C₁₋₄alkyl)-phenyl, —C(O)—NR^{A}R^{B}, —C(O)—(C₁₋₄alkyl), —C(O)—(C₃₋₆cycloalkyl),

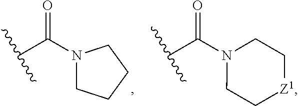

wherein Z¹ is selected from the group consisting of —CH₂—, —O—, and —NR_{c}—; wherein R^{A}, R^{B} and R^{C} are each independently selected from the group consisting of hydrogen and C₁₋₄alkyl; wherein each R¹¹ is independently selected from the group consisting of hydroxy, oxo, halogen, C₁₋₄alkyl, fluorinated C₁₋₄alkyl, C₁₋₄alkoxy, fluorinated C₁₋₄alkoxy, hydroxy substituted C₁₋₄alkyl, —(C₁₋₄alkyl)-phenyl, -cyano, —NR^{D}R^{E}, —C(O)—NR^{D}R^{E}, —C(O)—(C₁₋₄alkyl), —C(O)OH, and —C(O)O—(C₁₋₄alkyl), wherein R¹² is selected from the group consisting of hydroxy, oxo, halogen, C₁₋₂alkyl, CF₃, C₁₋₂alkoxy, —OCF₃ and hydroxy substituted C₁₋₂alkyl; m is an integer from 0 to 1; n is an integer from 0 to 1;

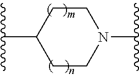

is selected from the group consisting of azetidin-3-yl, pyrrolidin-3-yl, pyrrolidin-3R-yl, pyrrolidin-3S-yl, and piperidin-4-yl; a is 1; L¹ is selected from the group consisting of —C(O)—, —C(O)—NR^{L}—, and —SO₂—, wherein R^{L} is selected from the group consisting of hydrogen and methyl; R³ is selected from the group consisting of C₂₋₄alkenyl, C₃₋₆cycloalkyl, and 5 to 6 membered saturated heterocyclyl; wherein the C₃₋₆cycloalkyl, 5 to 6 membered saturated heterocyclyl or 5 to 6 membered heteroaryl is optionally substituted with one to two substituents independently selected from the group consisting of hydroxy, cyano, C₁₋₄alkyl, fluorinated C₁₋₄alkyl, C₁₋₄alkoxy, fluorinated C₁₋₄alkoxy, and NR^{G}R^{H}; wherein R^{G} and R^{H} are each independently selected from the group consisting of hydrogen and C₁₋₄alkyl;

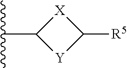

is selected from the group consisting of

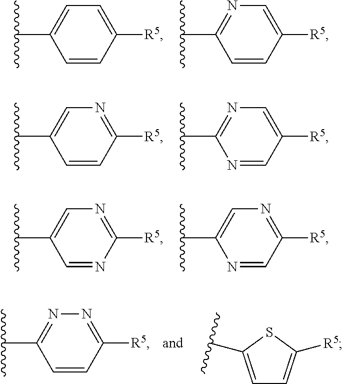

b is an integer from 0 to 1; each R⁴ is independently selected from the group consisting of halogen, C₁₋₄alkyl, fluorinated C₁₋₄alkyl, C₁₋₄alkoxy, fluorinated C₁₋₄alkoxy, and NR^{J}R^{K}, wherein R^{J} and R^{K} are each independently selected from the group consisting of hydrogen and C₁₋₂alkyl; provided that each R⁴ group is bound to a carbon atom; R⁵ is selected from the group consisting of (a)

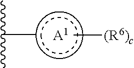

and

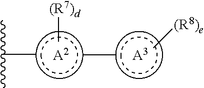

(b); wherein

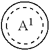

is selected from the group consisting of aryl, heteroaryl, and partially unsaturated heterocyclyl; c is an integer from 0 to 2; each R⁶ is independently selected from the group consisting of hydroxy, halogen, cyano, nitro, C₁₋₄alkyl, fluorinated C₁₋₄alkyl, hydroxy substituted C₁₋₄alkyl, C₁₋₄alkoxy, fluorinated C₁₋₄alkoxy, —NR^{M}R^{N}, —C(O)—(C₁₋₄alkyl), —C(O)—NR^{M}R^{N}, —C(O)OH, —C(O)O—(C₁₋₄alkyl), —NR^{M}—C(O)H, and —NR^{M}—SO₂—(C₁₋₄alkyl); wherein R^{M} and R^{N} are each independently selected from the group consisting of hydrogen and C₁₋₄alkyl; wherein

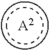

is selected from the group consisting of phenyl and 5 to 6 membered heteroaryl; d is an integer from 0 to 1; R⁷ is selected from the group consisting of hydroxy, halogen, cyano, C₁₋₄alkyl, fluorinated C₁₋₄alkyl, hydroxy substituted C₁₋₄alkyl, C₁₋₄alkoxy, and fluorinated C₁₋₄alkoxy; wherein

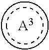

is selected from the group consisting of phenyl, 5 to 6 membered saturated heterocyclyl and 5 to 6 membered heteroaryl; e is an integer from 0 to 2; each R⁸ is independently selected from the group consisting of hydroxy, halogen, cyano, C₁₋₄alkyl, fluorinated C₁₋₄alkyl, hydroxy substituted C₁₋₄alkyl, C₁₋₄alkoxy, fluorinated C₁₋₄alkoxy, —NR^{T}R^{U}, —C(O)—NR^{T}R^{U}, —C(O)OH, —C(O)O—(C₁₋₄alkyl), and —(C₁₋₄alkyl)-NR^{T}R^{U}; provided that when

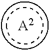

is a 5-membered heteroaryl, then

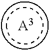

is bound at the 3-position, relative to the point of attachment of the

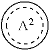

to the

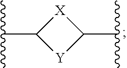

provided further that when

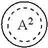

is phenyl or a 6 membered heteroaryl, then

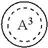

is bound at the 3- or 4-position, relative to the point of attachment of the

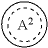

to the

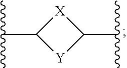

and a stereoisomer, a tautomer, and a pharmaceutically acceptable salt thereof.

In another embodiment, R¹ and R² are taken together to form an optionally substituted ring structure selected from the group consisting of (a) C₃₋₆cycloalkyl; and (c) 4 to 6 membered, saturated heterocyclyl; wherein the 4 to 6 membered saturated heterocyclyl contains NR¹⁰; provided that the NR¹⁰ is not present at the 2-position relative to the carbon atom of the imidazolidin-5-one; wherein R¹⁰ is selected from the group consisting of hydrogen, C₁₋₄alkyl, C₂₋₄alkenyl, —CH₂-(hydroxy substituted C₁₋₂alkyl), —CH₂-(phenyl), -(C₂alkyl)-O—(C₁₋₂alkyl), —C(O)—(C₁₋₄alkyl), —C(O)-(fluorinated C₁₋₂alkyl), —C(O)-(cyclopropyl), —C(O)O—(C₁₋₄alkyl), —C(O)—NR^{A}R^{B}, —SO₂—(C₁₋₂alkyl), wherein R and R are each independently selected from the group consisting of hydrogen and methyl; m is an integer from 0 to 1; and n is an integer from 0 to 2 provide that when n is 2 then m is 0;

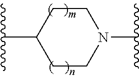

is selected from the group consisting of azetidin-3-yl, pyrrolidin-3-yl, pyrrolidin-3R-yl, pyrrolidin-3S-yl, piperidin-3-yl, piperidin-3R-yl, piperidin-3S-yl, and piperidin-4-yl; a is 1; L¹ is selected from the group consisting of —C(O)—, —C(O)O— and —SO₂—; R³ is selected from the group consisting of C₁₋₄alkyl, hydroxy substituted C₁₋₄alkyl, fluorinated C₁₋₂alkyl, C₂₋₄alkenyl, C₃₋₅ cycloalkyl, 4 to 5 membered, saturated heterocyclyl, 5 to 6 membered heteroaryl and NR^{V}R^{W}; wherein the C₃₋₅cycloalkyl, 4 to 5 membered, saturated heterocyclyl or 5 to 6 membered heteroaryl is optionally substituted with a substituent selected from the group consisting of halogen, hydroxy, C₁₋₂alkyl, (C₁₋₂alkyl)-OH, fluorinated C₁₋₂alkyl, cyano and NH₂; and wherein R^{V} and R^{W} are each independently selected from the group consisting of hydrogen and methyl;

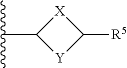

is selected from the group consisting of

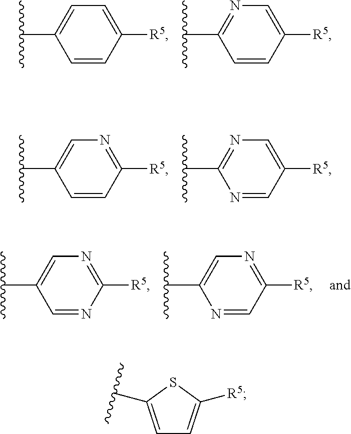

b is an integer from 0 to 1; R⁴ is selected from the group consisting of halogen, C₁₋₂ alkyl, and C₁₋₂alkoxy; R⁵ is selected from the group consisting of (a)

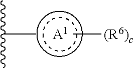

and

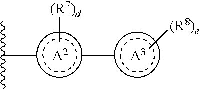

(b); wherein

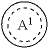

is selected from the group consisting of phenyl, naphthyl, 5 to 6 membered heteroaryl, 9 to 10 membered heteroaryl and partially unsaturated 9 to 10 membered heterocyclyl; c is an integer from 0 to 2; each R⁶ is independently selected from the group consisting of hydroxy, oxo, halogen, cyano, C₁₋₄ alkyl, fluorinated C₁₋₂alkyl, hydroxy substituted C₁₋₄alkyl, cyano-substituted C₁₋₂alkyl, —(C₁₋₂alkyl)-O—(C₁₋₂alkyl), C₁₋₄alkoxy, fluorinated C₁₋₂alkoxy, —SO₂—(C₁₋₄alkyl), —CO₂H, —C(O)O—(C₁₋₂alkyl), —C(O)—(C₁₋₂alkyl), —C(O)-(fluorinated C₁₋₂alkyl), —C(O)—NR^{M}R^{N}, —NR^{M}R^{N}, —NR^{M-}C(O)H—NR^{M}—SO₂—(C₁₋₂alkyl), C₃₋₅cycloalkyl, 1-cyano-cyclopropyl, —(C₁₋₂alkyl)-(C₃₋₅cycloalkyl), —S—(C₃₋₅ cycloalkyl), —SO₂—(C₃₋₅cycloalkyl), —NH—C(O)—(C₃₋₅ cycloalkyl) and —NH—SO₂—(C₃₋₅cycloalkyl) and oxetan-3-yl; and wherein R^{M} and R^{N} are each independently selected from the group consisting of hydrogen and C₁₋₂ alkyl; wherein

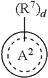

is selected from the group consisting of phenyl, 5 to 6 membered, saturated, nitrogen containing heterocyclyl and 5 to 6 membered nitrogen containing heteroaryl; wherein

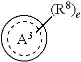

is selected from the group consisting of phenyl, 5 to 6 membered, saturated, nitrogen containing heterocyclyl and 5 to 6 membered, nitrogen containing heteroaryl; e is an integer from 0 to 1; R⁸ is selected from the group consisting of halogen, C₁₋₄alkyl, C₃₋₅cycloalkyl, —(C₁₋₂alkyl)-(C₃₋₅cycloalkyl) and oxetanyl; provided that the

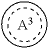

is bound at the 3- or 4-position of

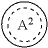

relative to the point of attachment of the

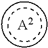

to the

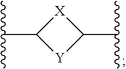

and a stereoisomer, a tautomer, and a pharmaceutically acceptable salt thereof.

In another embodiment, R¹ and R² are taken together to form an optionally substituted ring structure selected from the group consisting of (a) C₃₋₆cycloalkyl; and (c) 4 to 6 membered, saturated heterocyclyl; wherein the 4 to 6 membered saturated heterocyclyl contains NR¹⁰; provided that the NR¹⁰ is not present at the 2-position relative to the carbon atom of the imidazolidin-5-one; wherein R¹⁰ is selected from the group consisting of hydrogen, C₁₋₄alkyl, —CH₂-(hydroxy substituted C₁₋₂alkyl), —CH₂-(phenyl), —C(O)—(C₁₋₄alkyl), —C(O)-(cyclopropyl) and —C(O)—NR^{A}R^{B}; wherein R^{A} and R^{B} are each independently selected from the group consisting of hydrogen and methyl; m is an integer from 0 to 1; n is an integer from 0 to 1;

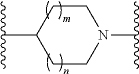

is selected from the group consisting of azetidin-3-yl, pyrrolidin-3R-yl, pyrrolidin-3S-yl, piperidin-3-yl, and piperidin-4-yl; a is 1; L¹ is selected from the group consisting of —C(O)— and —SO₂—; R³ is selected from the group consisting of C₂alkenyl, C₃cycloalkyl, 5-membered, saturated heterocyclyl and 5-membered heteroaryl; wherein the C₃cycloalkyl, 5-membered, saturated heterocyclyl or 5-membered heteroaryl is optionally substituted with a substituent selected from the group consisting of halogen, C₁₋₂alkyl, fluorinated C₁₋₂alkyl and cyano;

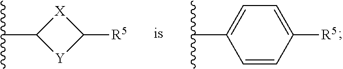

b is an integer from 0 to 1; R⁴ is independently selected from the group consisting of halogen, C₁₋₂alkyl, and C₁₋₂alkoxy, R⁵ is selected from the group consisting of (a)

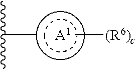

and

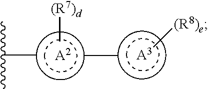

(b); wherein

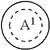

is selected from the group consisting of phenyl, heteroaryl, and partially unsaturated heterocyclyl; c is an integer from 0 to 2; each R⁶ is independently selected from the group consisting of hydroxy, halogen, cyano, C₁₋₂alkyl, fluorinated C₁₋₂alkyl, C₁₋₂alkoxy, fluorinated C₁₋₂alkoxy, —NR^{M}R^{N}, —C(O)—(C₁₋₂alkyl), —NR^{M}—C(O)H, and —NR^{M}—SO₂—(C₁₋₂alkyl); and wherein R^{M} and R^{N} are each independently selected from the group consisting of hydrogen and C₁₋₂alkyl; wherein

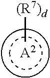

is phenyl; wherein

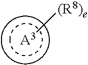

is selected from the group consisting of phenyl and 5 to 6 membered nitrogen containing heteroaryl; e is an integer from 0 to 1; R⁸ is selected from the group consisting of halogen and C₁₋₂alkyl; provided further that when

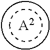

is phenyl, then

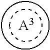

is bound at the 3- or 4-position, relative to the point of attachment of the

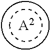

to the

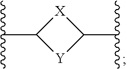

and a stereoisomer, a tautomer, and a pharmaceutically acceptable salt thereof.

In another embodiment, R¹ and R² are taken together to form a ring structure selected from the group consisting of cyclopropyl, cyclopentyl, piperidin-4,4-diyl, 1-(methyl)-piperidin-4,4-diyl, 1-(isopropyl)-piperidin-4,4-diyl, 1-(ethenyl)-piperidin-4,4-diyl, 1-(2-hydroxy-ethyl)-piperidin-4,4-diyl, 1-(methoxy-carbonyl)-piperidin-4,4-diyl, 1-(benzyl)-piperidin-4,4-diyl, 1-(methyl-carbonyl)-piperidin-4,4-diyl, 1-(isopropyl-carbonyl)-piperidin-4,4-diyl, 1-(trifluoromethyl-carbonyl)-piperidin-4,4-diyl, 1-(cyclopropyl-carbonyl)-piperidin-4,4-diyl, 1-(dimethylamino-carbonyl)-piperidin-4,4-diyl, 1-(methyl-sulfonyl)-piperidin-4,4-diyl, 1-(2-methoxy-ethyl)-piperidin-4,4-diyl, 1-(benzyl)-piperidin-4,4-diyl, tetrahydro-pyran-4,4-diyl, tetrahydro-furan-3,3-diyl and 1-(methoxycarbonyl)-azetidin-3,3-diyl; m is an integer from 0 to 1; and n is an integer from 0 to 2; provided that when n is 2 then m is 1;

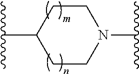

is selected from the group consisting of azetidin-3-yl, pyrrolidin-3-yl, pyrrolidin-3R-yl, pyrrolidin-3S-yl, piperidin-3-yl, piperidin-3R-yl, piperidin-3S-yl, and piperidin-4-yl; a is 1; L¹ is selected from the group consisting of —C(O)—, —C(O)O—, and —SO₂—; R³ is selected from the group consisting of methyl, ethyl, isopropyl, 1-hydroxyethyl, trifluoromethyl, 2,2,2-trifluoroethyl, 2-hydroxy-propan-2-yl, 3-hydroxy-2-methyl-propan-2-yl, ethenyl, cyclopropyl, 1-fluoro-cyclopropyl, 1-hydroxy-cyclopropyl, 1-hydroxymethyl-cyclopropyl, 1-methyl-cyclopropyl, 1-cyano-cyclopropyl, 1-amino-cyclopropyl, cyclobutyl, 1-methyl-cyclobutyl, amino, dimethylamino, pyrrolidin-1-yl, 1-methyl-pyrazol-3-yl, thiazol-2-yl, tetrahydro-furan-2-yl, tetrahydro-furan-2R-yl, oxetan-2-yl, oxetan-3-yl, 3-methyl-oxetan-3-yl, and pyridin-3-yl;

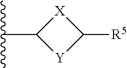

is selected from the group consisting of

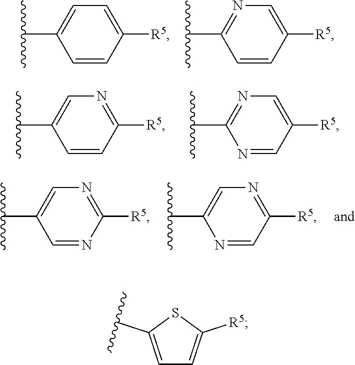

b is an integer from 0 to 1; R⁴ is selected from the group consisting of 2-fluoro, 3-fluoro, 2-chloro, 3-chloro, 2-methyl, 3-methyl and 2-methoxy; R⁵ is selected from the group consisting of (a)

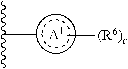

and

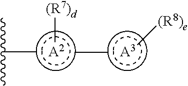

(b); wherein

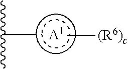

is selected from the group consisting of 3-cyano-phenyl, 4-cyano-phenyl, 3-hydroxy-phenyl, 4-hydroxy-phenyl, 3-fluoro-phenyl, 4-fluoro-phenyl, 3-chloro-phenyl, 4-chloro-phenyl, 2-fluoro-4-chloro-phenyl, 3-chloro-4-fluoro-phenyl, 2-fluoro-4-cyano-phenyl, 2-fluoro-4-(1-cyano-cyclopropyl)-phenyl, 2-fluoro-5-trifluoromethyl-phenyl, 2,4-dichloro-phenyl, 3-methyl-phenyl, 4-methyl-phenyl, 3-trifluoromethyl-phenyl, 4-trifluoromethyl-phenyl, 2-methoxy-phenyl, 3-methoxy-phenyl, 4-methoxy-phenyl, 3-trifluoromethoxy-phenyl, 4-trifluoromethoxy-phenyl, 4-(methylcarbonyl)-phenyl, 3-dimethylamino-phenyl, 4-dimethylamino-phenyl, 3-methylsulfonyl-amino-phenyl, 3-amino-4-hydroxy-phenyl, 3-formamido-4-hydroxy-phenyl 3-(cyclopropylthio)-phenyl, 3-(cyclopropylsulfonyl)-phenyl, 3-(cyclopropylcarbonyl-amino)-phenyl, 3-(cyclopropylsulfonyl-amino)-phenyl, 3-(methylsulfonyl)-phenyl, 3-(isopropylsulfonyl)-phenyl, 3-(aminocarbonyl)-phenyl, 3-carboxy-phenyl, 3-(methoxycarbonyl)-phenyl, naphth-2-yl, 6-fluoro-naphth-2-yl, 7-fluoro-naphth-2-yl, 8-fluoro-naphth-2-yl, 6-chloro-naphth-2-yl, 6-methyl-naphth-2-yl, 6-methoxy-naphth-2-yl, 8-methoxy-naphth-2-yl, 6-isopropyloxy-naphth-2-yl, 2-cyano-naphth-7-yl, 6-cyano-naphth-2-yl, 7-cyano-naphth-2-yl, 5-methoxy-naphth-2-yl, 7-methoxy-naphth-2-yl, 1,5-naphthyridin-3-yl, 1,8-naphthyridin-2-yl, 1,8-naphthyridin-3-yl, chroman-6-yl, isochroman-6-yl, isochroman-7-yl, pyridin-2-yl, pyridin-3-yl, pyridin-4-yl, 6-isopropyl-pyridin-3-yl, 6-n-propyl-pyridin-3-yl, 5-bromo-pyridin-2-yl, 5-chloro-pyridin-3-yl, 5-(2-hydroxy-2-methyl-propyl)-pyridin-2-yl, 5-(2-hydroxy-2-methyl-propyl)-pyridin-3-yl, 6-cyclopropyl-pyridin-3-yl, 6-(1-cyano-cyclopropyl)-pyridin-3-yl, 2-amino-pyrid-4-yl, 5-amino-pyridin-3-yl, 6-amino-pyridin-2-yl, 1-methyl-pyrazol-4-yl, 1-methyl-pyrazol-5-yl, indol-3-yl, indol-4-yl, indol-5-yl, indol-6-yl, 1-methyl-indol-5-yl, 1-methyl-indol-6-yl, 2-methyl-indol-5-yl, 2-hydroxymethyl-indol-5-yl, 3-(2-hydroxyethyl)-indol-5-yl, 3-cyanomethyl-indol-5-yl, 1,2-dimethyl-indol-5-yl, 1,3-dimethyl-indol-5-yl, 2,3-dimethyl-indol-5-yl, 1-methyl-3-(2-hydroxyethyl)-indol-5-yl, 1-(trifluoromethyl-carbonyl)-indol-5-yl, 2-oxo-indolin-5-yl, quinolin-2-yl, quinolin-3-yl, quinolin-5-yl, quinolin-6-yl, quinolin-7-yl, 2-chloro-quinolin-7-yl, 3-chloro-quinolin-7-yl, 4-chloro-quinolin-7-yl, 6-fluoro-quinolin-2-yl, 8-fluoro-quinolin-2-yl, 7-bromo-quinolin-2-yl, 2-hydroxy-quinolin-3-yl, 2-cyano-quinolin-6-yl, 2-cyano-quinolin-7-yl, 6-cyano-quinolin-2-yl, 2-methyl-quinolin-5-yl, 2-methyl-quinolin-6-yl, 2-methyl-quinolin-7-yl, 4-methyl-quinolin-7-yl, 2,4-dimethyl-quinolin-7-yl, 2-chloro-3-methyl-quinolin-7-yl, 2-chloro-4-methyl-quinolin-7-yl, 2-methyl-8-fluoro-quinolin-2-yl, 2-methyl-quinolin-7-yl, 2-methyl-7-bromo-quinolin-7-yl, 3-methyl-7-bromo-quinolin-7-yl, 2-methyl-4-chloro-quinolin-7-yl, 4-methyl-7-bromo-quinolin-2-yl, 2-trifluoromethyl-quinolin-7-yl, 2-oxo-quinolin-7-yl, 2-carboxy-quinolin-7-yl, 2-aminocarbonyl-quinolin-7-yl, isoquinolin-3-yl, isoquinolin-5-yl, isoquinolin-6-yl, isoquinolin-7-yl, 1-chloro-isoquinolin-6-yl, 3-chloro-isoquinolin-6-yl, 3-fluoro-isoquinolin-6-yl, 6-bromo-isoquinolin-3-yl, 1-methoxy-isoquinolin-6-yl, 3-methoxy-isoquinolin-6-yl, 1-amino-isoquinolin-6-yl, 3-amino-isoquinolin-6-yl, 1-oxo-isoquinolin-6-yl, quinazolin-7-yl, quinoxalin-6-yl, indazol-3-yl, indazol-4-yl, indazol-5-yl, indazol-6-yl, 4-chloro-indazol-5-yl, 1-methyl-indazol-3-yl, 1-methyl-indazol-4-yl, 1-methyl-indazol-5-yl, 1-methyl-indazol-6-yl, 2-methyl-indazol-4-yl, 2-methyl-indazol-5-yl, 2-methyl-indazol-6-yl, 1,3-dimethyl-indazol-5-yl, 1,4-dimethyl-indazol-5-yl, 1,7-dimethyl-indazol-5-yl, 1,8-dimethyl-indazol-5-yl, 1-ethyl-indazol-5-yl, 2-ethyl-indazol-5-yl, 1-isopropyl-indazol-5-yl, 2-isopropyl-indazol-5-yl, 1-(2-hydroxyethyl)-indazol-5-yl, 2-(2-hydroxyethyl)-indazol-5-yl, 1-(2-hydroxyethyl)-6-fluoro-indazol-5-yl, 2-(2-hydroxyethyl)-6-fluoro-indazol-5-yl, 1-methyl-3-chloro-indazol-5-yl, 1-methyl-3-chloro-indazol-6-yl, 1-methyl-3-amino-indazol-6-yl, 1-methyl-3-aminocarbonyl-indazol-6-yl, 1-methyl-3-cyano-indazol-5-yl, 1-methyl-3-cyano-indazol-6-yl, 1-methyl-3-methoxy-indazol-5-yl, 1-methyl-3-methoxymethyl-indazol-5-yl, 1-methyl-3-methoxymethyl-indazol-6-yl, 1-methyl-7-methoxymethyl-indazol-4-yl, 1-methyl-3-hydroxymethyl-indazol-5-yl, 1-methyl-3-hydroxymethyl-indazol-6-yl, 1-methyl-7-hydroxymethyl-indazol-4-yl, 1-methyl-3-cyclopropyl-indazol-5-yl, 2-methyl-3-cyano-indazol-5-yl, 2-methyl-3-hydroxymethyl-indazol-5-yl, 2-methyl-3-methoxymethyl-indazol-5-yl, 1-(2-hydroxyethyl)-indazol-5-yl, 2-(2-hydroxyethyl)-indazol-5-yl), 1-(2-cyanoethyl)-indazol-5-yl, 2-(2-cyanoethyl)-indazol-5-yl, 1-oxetan-3-yl-indazol-5-yl, 1-cyclopropyl-indazol-5-yl, 1-cyclopropylmethyl-indazol-5-yl, 2-cyclopropylmethyl-indazol-5-yl, benzofuran-5-yl, benzofuran-6-yl, 2-methyl-benzofuran-5-yl, 2,3-dimethyl-benzofuran-5-yl, 2-cyano-benzofuran-5-yl, benzimidazol-2-yl, benzimidazol-5-yl, 1-methyl-benzimidazol-2-yl, 1,2-dimethyl-benzimidazol-6-yl, 1-methyl-6-fluoro-benzimidazol-2-yl, 2-oxo-benzimidazol-5-yl, benzoxazol-2-yl, benzoxazol-5-yl, 6-chloro-benzoxazol-2-yl, benzisoxazol-5-yl, benzthiazol-2-yl, benzthiazol-5-yl, 5-fluoro-benzothiazol-2-yl, 6-fluoro-benzothiazol-2-yl, 5-chloro-benzothiazol-2-yl, 6-chloro-benzothiazol-2-yl, 5,6-difluoro-benzothiazol-2-yl, 2-methyl-benzothiazol-5-yl, 2-methyl-benzothiazol-6-yl, 6-methyl-benzothiazol-2-yl, 2-methyl-benzothiazol-5-yl, 5-cyano-benzothiazol-2-yl, 6-cyano-benzothiazol-2-yl, benzothien-5-yl, 2-methyl-benzothien-5-yl, 2,3-dimethyl-benzothien-5-yl, 2,3-dihydro-benzofuran-5-yl, 2-oxo-3,4-dihydro-quinolin-7-yl, 1,2,3,4-tetrahydro-2-methylcarbonyl-isoquinolin-6-yl, 1,2,3,4,4a,8a-hexahydro-2-methyl-carbonyl-isoquinolin-6-yl, 2,3-dihydro-benzo[1,4]dioxin-6-yl, 2,3-dihydrobenzofuran-5-yl, 1,2-dimethyl-1,2-dihydro-3-oxo-indazol-5-yl, 2-oxo-3,4-dihydro-quinolin-6-yl, benzo[1,3]dioxol-5-yl, pyrrolo[2,3-b]pyridin-5-yl, 1-methyl-pyrazolo[4,3-b]pyridin-5-yl, [1,2,4]triazo[4,3-a]pyridin-6-yl, 3-methyl-[1,2,4]triazo[4,3-a]pyridin-6-yl and 4-methyl-3,4-dihydro-pyrido[3,2-b][1,4]oxazin-7-yl;

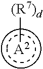

is selected from the group consisting of phenyl, pyridine-3-yl, and pyridine-4-yl; and

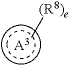

is selected from the group consisting of 4-bromo-phenyl, 3-chloro-phenyl, 4-methyl-phenyl, pyridin-3-yl, pyridin-4-yl, 1-methyl-pyrazol-3-yl, 1-methyl-pyrazol-4-yl, 1-methyl-pyrazol-5-yl, 1-isopropyl-pyrazol-4-yl, 1-isobutyl-pyrazol-5-yl, 1-(2-methylpropyl)-pyrazol-3-yl, 1-cyclopropyl-pyrazol-4-yl, 1-cyclobutyl-pyrazol-4-yl, 1-cyclopropylmethyl-pyrazol-3-yl, 1-cyclopropylmethyl-pyrazol-5-yl, 1,2,3,4-tetrazol-5-yl, pyrazol-3-yl, pyrrolidin-1-yl, morpholin-4-yl, 4-methyl-piperazin-1-yl, imidazol-1-yl, piperazin-1-yl, 4-methyl-piperazin-1-yl, and 1-(oxetan-3-yl)-pyrazol-4-yl; provided that when

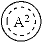

is a phenyl or pyridine-3-yl, then

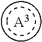

is bound to

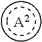

at the 4-position, relative to the point of attachment of the

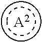

to the

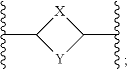

provided further that when

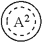

is a phenyl or pyridine-4-yl, then

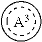

is bound to

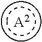

at the 3-position, relative to the point of attachment of the

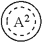

to the

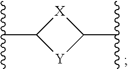

and a stereoisomer, a tautomer, and a pharmaceutically acceptable salt thereof.

In another embodiment, R¹ and R² are taken together to form a ring structure selected from the group consisting of cyclopropyl, cyclopentyl, piperidin-4,4-diyl, 1-(methyl)-piperidin-4,4-diyl, 1-(isopropyl)-piperidin-4,4-diyl, 1-(2-hydroxy-ethyl)-piperidin-4,4-diyl, 1-(methoxy-carbonyl)-piperidin-4,4-diyl, 1-(benzyl)-piperidin-4,4-diyl, 1-(methyl-carbonyl)-piperidin-4,4-diyl, 1-(isopropyl-carbonyl)-piperidin-4,4-diyl, 1-(cyclopropyl-carbonyl)-piperidin-4,4-diyl and 1-(dimethylamino-carbonyl)-piperidin-4,4-diyl; m is an integer from 0 to 1; n is an integer from 0 to 1;

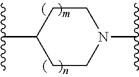

is selected from the group consisting of azetidin-3-yl, pyrrolidin-3R-yl, pyrrolidin-3S-yl, and piperidin-4-yl; a is 1; L¹ is selected from the group consisting of —C(O)— and —SO₂—; R³ is selected from the group consisting of 2,2,2-trifluoroethyl, ethenyl, cyclopropyl, 1-fluoro-cyclopropyl, 1-methyl-cyclopropyl, 1-cyano-cyclopropyl, pyrrolidin-1-yl, 1-methyl-pyrazol-3-yl and tetrahydro-furan-2-yl;

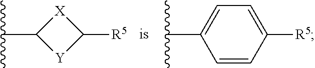

b is an integer from 0 to 1; R⁴ is selected from the group consisting of 2-fluoro, 2-methyl, 3-methyl and 2-methoxy; R⁵ is selected from the group consisting of (a)

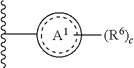

and

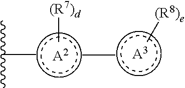

(b); wherein

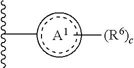

is selected from the group consisting of 4-(3-cyano-phenyl), 4-(4-cyano-phenyl), 4-(3-hydroxy-phenyl), 4-(4-hydroxy-phenyl), 4-(3-fluoro-phenyl), 4-(4-fluoro-phenyl), 4-(3-chloro-phenyl), 4-(4-chloro-phenyl), 4-(2,4-dichloro-phenyl), 4-(3-methyl-phenyl), 4-(4-methyl-phenyl), 4-(3-trifluoromethyl-phenyl), 4-(4-trifluoromethyl-phenyl), 4-(2-methoxy-phenyl), 4-(3-methoxy-phenyl), 4-(4-methoxy-phenyl), 4-(3-trifluoromethoxy-phenyl), 4-(4-trifluoromethoxy-phenyl), 4-(3-dimethylamino-phenyl), 4-(4-dimethylamino-phenyl), 4-(3-methylsulfonyl-amino-phenyl), 4-(3-amino-4-hydroxy-phenyl), 4-(3-formamido-4-hydroxy-phenyl), 4-(pyridin-2-yl), 4-(pyridin-3-yl), 4-(pyridin-4-yl), 4-(1-methyl-pyrazol-4-yl), 4-(1-methyl-pyrazol-5-yl), 4-(indol-4-yl), 4-(indol-5-yl), 4-(indol-6-yl), 4-(quinolin-5-yl), 4-(quinolin-6-yl), 4-(isoquinolin-5-yl), 4-(isoquinolin-6-yl), 4-(isoquinolin-7-yl), 4-(indazol-4-yl), 4-(indazol-5-yl), 4-(1-methyl-indazol-5-yl), 4-(1-methyl-indazol-6-yl), 4-(benzofuran-5-yl), 4-(2-methyl-benzofuran-5-yl), 4-(benzimidazol-5-yl), 4-(benzoxazol-2-yl), 4-(benzoxazol-5-yl), 4-(benzthiazol-5-yl), 4-(2,3-dimethyl-benzothiophen-5-yl), 4-(1,2,3,4-tetrahydro-2-methylcarbonyl-isoquinolin-6-yl) and 4-(1,2,3,4,4a,8a-hexahydro-2-methyl-carbon-isoquinolin-6-yl);

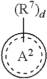

is 4-(phenyl); and

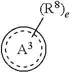

is selected from the group consisting of 4-(4-bromo-phenyl), 4-(pyridin-3-yl), 4-(pyridin-4-yl), 4-(1-methyl-pyrazol-4-yl), 4-(1-methyl-pyrazol-5-yl), 4-(tetrazol-5-yl) and 3-(pyrazol-3-yl); and a stereoisomer, a tautomer, and a pharmaceutically acceptable salt thereof.

In another embodiment, R¹ and R² are taken together to form a ring structure selected from the group consisting of cyclopropyl, cyclopentyl, piperidin-4,4-diyl, 1-(methyl)-piperidin-4,4-diyl, 1-(isopropyl)-piperidin-4,4-diyl, 1-(2-hydroxy-ethyl)-piperidin-4,4-diyl, 1-(ethenylcarbonyl)-piperidin-4,4-diyl, 1-(trifluoromethyl-carbonyl)piperidin-4,4-diyl, 1-(methoxy-carbonyl)-piperidin-4,4-diyl, 1-(2-methoxyethyl)-piperidin-4,4-diyl, 1-(benzyl)-piperidin-4,4-diyl, 1-(methyl-carbonyl)-piperidin-4,4-diyl, 1-(isopropyl-carbonyl)-piperidin-4,4-diyl, 1-(cyclopropyl-carbonyl)-piperidin-4,4-diyl, 1-(methylsulfonyl)-piperidin-4,4-diyl, 1-(dimethylamino-carbonyl)-piperidin-4,4-diyl, 1-(methoxycarbonyl)-azetidin-3,3-diyl, tetrahyrdofuran-3,3-diyl and tetrahydro-pyran-4,4-diyl; m is an integer from 0 to 1; and n is an integer from 0 to 1;

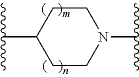

is selected from the group consisting of azetidin-3-yl, pyrrolidin-3R-yl, pyrrolidin-3S-yl and piperidin-4-yl; a is 1; L¹ is —C(O)—; R³ is selected from the group consisting of ethyl, 1-hydroxy-ethyl, isopropyl, 2-hydroxy-propan-2-yl, 3-hydroxy-2-methyl-propan-2-yl, 2,2,2-trifluoroethyl, ethenyl, cyclopropyl, 1-fluoro-cyclopropyl, 1-methyl-cyclopropyl, 1-hydroxy-cyclopropyl, 1-hydroxymethyl-cyclopropyl, 1-amino-cyclopropyl, cyclobutyl, 1-methyl-cyclobutyl, pyrrolidin-1-yl, 1-methyl-pyrazol-3-yl, oxetan-2-yl, oxetan-3yl, 3-methyl-oxetan-3-yl, tetrahydro-furan-2yl, tetrahydro-furan-2R-yl, tetrahydro-furan-2-yl and dimethylamino;

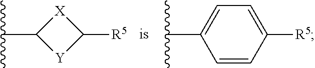

b is an integer from 0 to 1; R⁴ is selected from the group consisting of 2-fluoro, 2-chloro, 2-methyl, 2-methoxy, 3-fluoro and 3-methyl; R⁵ is

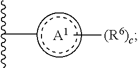

wherein

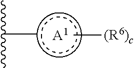

is selected from the group consisting of 4-cyano-phenyl, 3-hydroxy-phenyl, 4-hydroxy-phenyl, 3-fluoro-phenyl, 4-fluoro-phenyl, 3-chloro-phenyl, 4-chloro-phenyl, 2,4-dichloro-phenyl, 2-fluoro-4-chloro-phenyl, 3-chloro-4-fluoro-phenyl, 3-methyl-phenyl, 4-methyl-phenyl, 3-trifluoromethyl-phenyl, 3-methoxy-phenyl, 4-methoxy-phenyl, 3-aminocarbonyl-phenyl, 3-dimethylamino-phenyl, 4-dimethylamino-phenyl, 3-methylsulfonyl-amino-phenyl, 3-(cyclopropyl-sulfonylamino)-phenyl, 3-(cyclopropyl-carbonylamino)-phenyl, 3-(cyclopropyl-thio)-phenyl, 3-(cyclopropyl-sulfonyl)-phenyl, naphtha-2-yl, 6-fluoro-naphth-2-yl, 8-fluoro-naphth-2-yl, 6-chloro-naphth-2-yl, 7-fluoro-naphth-2-yl, 8-fluoro-naphth-2-yl, 6-methyl-naphth-2-yl, 5-methoxy-naphth-2-yl, 6-methoxy-naphth-2-yl, 8-methoxy-naphth-2-yl, 6-isopropoxy-naphth-2-yl, 6-cyano-naphth-2-yl, 7-methoxy-naphth-2-yl, 7-cyano-naphth-2-yl, 6-amino-pyridin-2-yl, isochroman-6-yl, isochroman-7-yl, 2-oxo-indolin-5-yl, indol-3-yl, indol-4-yl, indol-5-yl, indol-6-yl, 1-methyl-indol-5-yl, 1-methyl-indol-6-yl, 2-methyl-indol-5-yl, 1,2-dimethyl-indol-5-yl, 1,3-dimethyl-indol-5-yl, 2,3-dimethyl-indol-5-yl, 2-hydroxymethyl-indol-5-yl, 3-(2-hydroxyethyl-indol-5-yl), 3-cyanomethyl-indol-5-yl, 1-methyl-3-(2-hydroxyethyl)-indol-5-yl, quinolin-2-yl, quinolin-3-yl, quinolin-5-yl, quinolin-6-yl, quinolin-7-yl, 2-chloro-quinolin-7-yl, 4-chloro-quinolin-7-yl, 6-fluoro-quinolin-2-yl, 8-fluoro-quinolin-2-yl, 3-chloro-quinolin-7-yl, 2-methyl-quinolin-6-yl, 2-methyl-quinolin-6-yl, 4-methyl-quinolin-7-yl, 2-cyano-quinolin-6-yl, 2-chloro-3-methyl-quinolin-7-yl, isoquinolin-3-yl, isoquinolin-5-yl, isoquinolin-6-yl, isoquinolin-7-yl, 3-fluoro-isoquinolin-6-yl, 1-chloro-isoquinolin-6-yl, 3-chloro-isoquinolin-6-yl, 1-methoxy-isoquinolin-6-yl, 3-methoxy-isoquinolin-6-yl, 1-amino-isoquinolin-6-yl, 3-amino-isoquinolin-6-yl, 1-oxo-isoquinolin-6-yl, quinazolin-7-yl, indazol-3-yl, indazol-4-yl, indazol-5-yl, indazol-6-yl, 1-methyl-indazol-5-yl, 1-methyl-indazol-6-yl, 1-methyl-indazol-3-yl, 1-methyl-indazol-4-yl, 1-methyl-indazol-5-yl, 1-methyl-indazol-6-yl, 2-methyl-indazol-4-yl, 2-methyl-indazol-5-yl, 2-methyl-indazol-6-yl, 1,3-dimethyl-indazol-5-yl, 1,4-dimethyl-indazol-5-yl, 1,8-dimethyl-indazol-5-yl, 1-ethyl-indazol-5-yl, 1-methyl-3-chloro-indazol-5-yl, 1-methyl-3-chloro-indazol-6-yl, 1-methyl-3-aminocarbonyl-indazol-6-yl, 1-methyl-3-cyano-indazol-6-yl, 1-methyl-3-amino-indazol-6-yl, 1-methyl-3-methoxy-indazol-5-yl, 1-methyl-3-methoxymethyl-indazol-5-yl, 1-methyl-3-methoxymethyl-indazol-6-yl, 1-methyl-3-hydroxymethyl-indazol-5-yl, 1-methyl-3-hydroxymethyl-indazol-6-yl, 1-methyl-3-cyclopropyl-indazol-5-yl, 1-(cyclopropylmethyl)-indazol-5-yl, benzofuran-5-yl, benzofuran-6-yl, 2-methyl-benzofuran-5-yl, 2-cyano-benzofuran-5-yl, 2,3-dimethyl-benzofuran-5-yl, benzoxazol-2-yl, benzoxazol-5-yl, 6-chloro-benzoxazol-2-yl, benzimidazol-2-yl, benzimidazol-5-yl, 1-methyl-benzimidazol-5-yl, 2-oxo-benzimidazol-5-yl, benzothiazol-2-yl, benzthiazol-5-yl, 5-chloro-benzothiazol-2-yl, 5-fluoro-benzothiazol-2-yl, 6-fluoro-benzothiazol-2-yl, 6-chloro-benzothiazol-2-yl, 5,6-difluoro-benzothiazol-2-yl, 2-methyl-benzothiazol-5-yl, 2-methyl-benzothiazol-6-yl, 5-cyano-benzothiazol-2-yl, 6-cyano-benzthiazol-2-yl, benzothien-5-yl, 2-methyl-benzothien-5-yl, 2,3-dimethyl-benzothien-5-yl, 2,3-dihydrobenzofuran-5-yl, 2-oxo-3,4-dihydro-quinolin-6-yl, benzo[1,3]dioxol-5-yl, 1,8-naphthyridin-2-yl and pyrrolo[2,3-b]pyridin-5-yl; and a stereoisomer, a tautomer, and a pharmaceutically acceptable salt thereof.

In another embodiment, R¹ and R² are taken together to form a ring structure selected from the group consisting of cyclopropyl, cyclopentyl, piperidin-4,4-diyl, 1-(methyl)-piperidin-4,4-diyl, 1-(isopropyl)-piperidin-4,4-diyl, 1-(2-hydroxy-ethyl)-piperidin-4,4-diyl, 1-(methoxy-carbonyl)-piperidin-4,4-diyl, 1-(benzyl)-piperidin-4,4-diyl, 1-(methyl-carbonyl)-piperidin-4,4-diyl, 1-(isopropyl-carbonyl)-piperidin-4,4-diyl, 1-(cyclopropyl-carbonyl)-piperidin-4,4-diyl and 1-(dimethylamino-carbonyl)-piperidin-4,4-diyl; m is an integer from 0 to 1; n is an integer from 0 to 1;

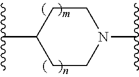

is selected from the group consisting of azetidin-3-yl, pyrrolidin-3R-yl, pyrrolidin-3S-yl, and piperidin-4-yl; a is 1; L¹ is —C(O)—; R³ is selected from the group consisting of 2,2,2-trifluoroethyl, ethenyl, cyclopropyl, 1-methyl-cyclopropyl, pyrrolidin-1-yl and 1-methyl-pyrazol-3-yl;

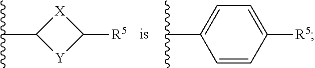

b is an integer from 0 to 1; R⁴ is selected from the group consisting of 2-fluoro, 2-methyl, 3-methyl, and 2-methoxy; R⁵ is

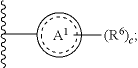

wherein

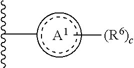

is selected from the group consisting of 4-(4-cyano-phenyl), 4-(3-hydroxy-phenyl), 4-(4-hydroxy-phenyl), 4-(3-fluoro-phenyl), 4-(4-fluoro-phenyl), 4-(3-chloro-phenyl), 4-(4-chloro-phenyl), 4-(2,4-dichloro-phenyl), 4-(3-methyl-phenyl), 4-(4-methyl-phenyl), 4-(3-trifluoromethyl-phenyl), 4-(3-methoxy-phenyl), 4-(4-methoxy-phenyl), 4-(3-dimethylamino-phenyl), 4-(4-dimethylamino-phenyl), 4-(3-methylsulfonyl-amino-phenyl), 4-(indol-4-yl), 4-(indol-5-yl), 4-(indol-6-yl), 4-(quinolin-5-yl), 4-(quinolin-6-yl), 4-(isoquinolin-5-yl), 4-(isoquinolin-6-yl), 4-(isoquinolin-7-yl), 4-(indazol-4-yl), 4-(indazol-5-yl), 4-(1-methyl-indazol-5-yl), 4-(1-methyl-indazol-6-yl), 4-(benzofuran-5-yl), 4-(2-methyl-benzofuran-5-yl), 4-(benzoxazol-2-yl), 4-(benzoxazol-5-yl), 4-(benzthiazol-5-yl) and 4-(2,3-dimethyl-benzothiophen-5-yl); and a stereoisomer, a tautomer, and a pharmaceutically acceptable salt thereof.

In some embodiments, R¹ and R² are taken together to form a ring structure selected from the group consisting of cyclopropyl, cyclopentyl, piperidin-4,4-diyl, 1-(methyl)-piperidin-4,4-diyl, 1-(isopropyl)-piperidin-4,4-diyl, 1-(2-hydroxy-ethyl)-piperidin-4,4-diyl, 1-(methoxy-carbonyl)-piperidin-4,4-diyl, 1-(benzyl)-piperidin-4,4-diyl, 1-(methyl-carbonyl)-piperidin-4,4-diyl, 1-(isopropyl-carbonyl)-piperidin-4,4-diyl, 1-(cyclopropyl-carbonyl)-piperidin-4,4-diyl, 1-(dimethylamino-carbonyl)-piperidin-4,4-diyl, 1-(trifluoromethyl-carbonyl)-piperidin-4,4-diyl, 1-(methyl-sulfonyl)-piperidin-4,4-diyl, 1-(2-methoxyethyl)-piperidin-4,4-diyl, 1-(methoxycarbonyl)azetidin-3,3-diyl, tetrahydro-furan-3,3-diyl, tetrahydro-pyran-4,4-diyl; m is an integer from 0 to 1; and n is an integer from 0 to 1;

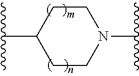

is selected from the group consisting of azetidin-3-yl, pyrrolidin-3R-yl, and piperidin-4-yl; a is 1; L is —C(O)—; R³ is selected from the group consisting of ethyl, cyclopropyl, 1-hydroxy-cyclopropyl, 1-fluoro-cyclopropyl, 1-methyl-cyclopropyl, 1-hydroxymethyl-cyclopropyl, cyclobutyl, tetrahydro-furan-2-yl, tetrahydro-furan-2R-yl, tetrahydro-furan-2S-yl, and oxetan-2-yl;

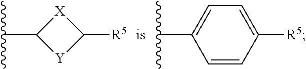

b is an integer from 0 to 1; R⁴ is selected from the group consisting of 2-fluoro, 2-chloro, 2-methyl, 2-methoxy, 3-fluoro and 3-methyl; R⁵ is

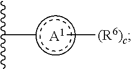

wherein

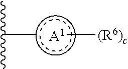

is selected from the group consisting of 4-cyano-phenyl, 3-hydroxy-phenyl, 4-fluoro-phenyl, 3-chloro-phenyl, 4-chloro-phenyl, 2-fluoro-4-chloro-phenyl, 3-chloro-4-fluoro-phenyl, 2-fluoro-4-cyano-phenyl, 2,4-dichloro-phenyl, 3-methyl-phenyl, 4-methyl-phenyl, 3-methoxy-phenyl, 4-methoxy-phenyl, 4-dimethylamino-phenyl, 3-(cyclopropyl-sulfonylamino)-phenyl, 3-(cyclopropyl-carbonylamino)-phenyl, 3-(cyclopropyl-thio)-phenyl, naphth-2-yl, 6-fluoro-naphth-2-yl, 7-fluoro-naphth-2-yl, 8-fluoro-naphth-2-yl, 6-chloro-naphth-2-yl, 6-methyl-naphth-2-yl, 6-methoxy-naphth-2-yl, 8-methoxy-naphth-2-yl, 6-cyano-naphth-2-yl, indol-3-yl, indol-4-yl, indol-5-yl, indol-6-yl, 1-methyl-indol-5-yl, 1-methyl-indol-6-yl, 2-methyl-indol-5-yl, 2,3-dimethyl-indol-5-yl, 2-(hydroxymethyl)-indol-5-yl, 3-(2-hydroxyethyl)-indol-5-yl, 3-(cyanomethyl)-indol-5-yl, 1-methyl-3-(2-hydroxyethyl)-indol-5-yl, 2-oxo-indolin-5-yl, quinolin-2-yl, quinolin-3-yl, quinolin-5-yl, quinolin-6-yl, quinolin-7-yl, 3-chloro-quinolin-7-yl, 6-fluoro-quinolin-2-yl, 8-fluoro-quinolin-2-yl, 8-fluoro-quinolin-7-yl, 4-methyl-quinolin-7-yl, 2-cyano-quinolin-6-yl, isoquinolin-5-yl, isoquinolin-6-yl, isoquinolin-7-yl, 6-fluoro-isoquinolin-6-yl, 1-amino-isoquinolin-6-yl, 3-amino-isoquinolin-6-yl, quinazolin-7-yl, indazol-3-yl, indazol-4-yl, indazol-5-yl, indazol-6-yl, 1-methyl-indazol-4-yl, 1-methyl-indazol-5-yl, 1-methyl-indazol-6-yl, 2-methyl-indazol-6-yl, 1,3-dimethyl-indazol-5-yl, 1,4-dimethyl-indazol-5-yl, 1-methyl-3-amino-indazol-6-yl, 1-methyl-3-aminocarbonyl-indazol-6-yl, 1-methyl-3-methoxymethyl-indazol-5-yl, 1-methyl-3-methoxymethyl-indazol-6-yl, 1-methyl-3-cyclopropyl-indazol-5-yl, benzofuran-5-yl, 2-methyl-benzofuran-5-yl, 2-cyano-benzofuran-5-yl, 2,3-dimethyl-benzofuran-5-yl, benzothiazol-2-yl, benzothiazol-5-yl, 6-fluoro-benzothiazol-2-yl, 6-chloro-benzothiazol-2-yl, 2-methyl-benzothiazol-5-yl, 6-methyl-benzothiazol-2-yl, 6-cyano-benzothiazol-2-yl, benzoxazol-2-yl, benzimidazol-5-yl, 1-methyl-benzimidazol-5-yl, benzothien-5-yl, 2-methyl-benzothien-5-yl, 2,3-dimethyl-benzothien-5-yl, and pyrrolo[2,3-b]pyridin-5-yl; and a stereoisomer, a tautomer and a pharmaceutically acceptable salt thereof.

In another embodiment, R¹ and R² are taken together to form a ring structure selected from the group consisting of cyclopropyl, cyclopentyl, piperidin-4,4-diyl, 1-(methyl)-piperidin-4,4-diyl, 1-(2-hydroxy-ethyl)-piperidin-4,4-diyl, 1-(methoxy-carbonyl)-piperidin-4,4-diyl, 1-(benzyl)-piperidin-4,4-diyl, 1-(methyl-carbonyl)-piperidin-4,4-diyl, 1-(isopropyl-carbonyl)-piperidin-4,4-diyl, I-(cyclopropyl-carbonyl)-piperidin-4,4-diyl and 1-(dimethylamino-carbonyl)-piperidin-4,4-yl; m is an integer from 0 to 1; n is an integer from 0 to 1;

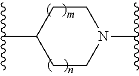

is selected from the group consisting of azetidin-3-yl, pyrrolidin-3R-yl, and piperidin-4-yl; a is 1; L¹ is —C(O)—; R³ is selected from the group consisting of cyclopropyl and 1-methyl-cyclopropyl;

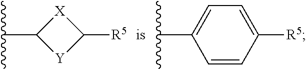

b is an integer from 0 to 1; R⁴ is selected from the group consisting of 2-fluoro, 2-methyl, 3-methyl, and 2-methoxy; R⁵ is

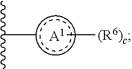

wherein

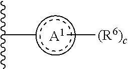

is selected from the group consisting of 4-(4-cyano-phenyl), 4-(3-hydroxy-phenyl), 4-(4-fluoro-phenyl), 4-(3-chloro-phenyl), 4-(4-chloro-phenyl), 4-(2,4-dichloro-phenyl), 4-(3-methyl-phenyl), 4-(4-methyl-phenyl), 4-(3-methoxy-phenyl), 4-(4-methoxy-phenyl), 4-(4-dimethylamino-phenyl), 4-(indol-4-yl), 4-(indol-5-yl), 4-(indol-6-yl), 4-(isoquinolin-5-yl), 4-(isoquinolin-6-yl), 4-(isoquinolin-7-yl), 4-(indazol-4-yl), 4-(indazol-5-yl), 4-(1-methyl-indazol-5-yl), 4-(1-methyl-indazol-6-yl), 4-(benzofuran-5-yl), 4-(2-methyl-benzofuran-5-yl), 4-(benzthiazol-5-yl) and 4-(2,3-dimethyl-benzothiophen-5-yl); and a stereoisomer, a tautomer, and a pharmaceutically acceptable salt thereof.

In some embodiments, R¹ and R² are taken together to form a ring structure selected from the group consisting of cyclopropyl, cyclopentyl, 1-(methoxy-carbonyl)-piperidin-4,4-diyl, 1-(isopropyl-carbonyl)-piperidin-4,4-diyl, and 1-(dimethylamino-carbonyl)-piperidin-4,4-diyl; m is an integer from 0 to 1; and n is 0;

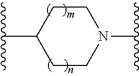

is selected from the group consisting of azetidin-3-yl and pyrrolidin-3R-yl; a is 1; L¹ is —C(O)—; R³ is selected from the group consisting of cyclopropyl, 1-fluoro-cyclopropyl, 1-hydroxy-cyclopropyl, 1-methyl-cyclopropyl, tetrahydrfuran-2S-yl and oxetan-2-yl;

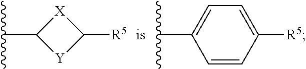

b is an integer from 0 to 1; R⁴ is selected from the group consisting of 2-fluoro, 2-chloro, and 2-methyl; R⁵ is

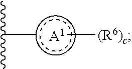

wherein

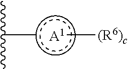

is selected from the group consisting of 3-hydroxy-phenyl, naphth-2-yl, 6-fluoro-naphth-2-yl, 7-fluoro-naphth-2-yl, 8-fluoro-naphth-2-yl, 6-chloro-naphth-2-yl, 6-methyl-naphth-2-yl, 6-methoxy-naphth-2-yl, 8-methoxy-naphth-2-yl, 6-cyano-naphth-2-yl, indol-3-yl, indol-5-yl, indol-6-yl, 1-methyl-indol-5-yl, 2-methyl-indol-5-yl, 2,3-dimethyl-indol-5-yl, 3-cyanomethyl-indol-5-yl, 2-hydroxymethyl-indol-5-yl, 3-(2-hydroxyethyl)-indol-5-yl, quinolin-3-yl, quinolin-5-yl, quinolin-7-yl, 3-chloro-quinolin-7-yl, 6-fluoro-quinolin-2-yl, 8-fluoro-quinolin-2-yl, 2-cyano-quinolin-6-yl, isoquinolin-6-yl, indazol-4-yl, indazol-5-yl, indazol-6-yl, 1-methyl-indazol-5-yl, 2-methyl-indazol-6-yl, benzofuran-5-yl, 2-methyl-benzofuran-5-yl, 2-cyano-benzofuran-5-yl, benzothiazol-2-yl, benzthiazol-5-yl, 6-chloro-benzothiazol-2-yl, 6-methyl-benzothiazol-2-yl, 6-cyano-benzothiazol-2-yl, benzoxazol-2-yl, benzimidazol-5-yl, 1-methyl-benzimidazol-5-yl, benzothien-5-yl, 2-methyl-benzothien-5-yl, and 2,3-dimethyl-benzothien-5-yl; and a stereoisomer, a tautomer, and a pharmaceutically acceptable salt thereof.

In another embodiment, R¹ and R² are taken together to form a ring structure selected from the group consisting of cyclopropyl, cyclopentyl, 1-(methoxy-carbonyl)-piperidin-4,4-diyl, 1-(isopropyl-carbonyl)-piperidin-4,4-diyl, and 1-(dimethylamino-carbonyl)-piperidin-4,4-diyl; m is an integer from 0 to 1; n is 0;

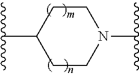

is selected from the group consisting of azetidin-3-yl and pyrrolidin-3R-yl; a is 1; L¹ is —C(O)—; R³ is a cyclopropyl and 1-methyl-cyclopropyl;

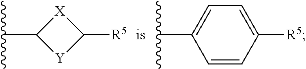

b is an integer from 0 to 1; R⁴ is selected from the group consisting of 2-fluoro and 2-methyl; R⁵ is

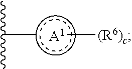

wherein

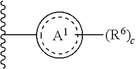

is selected from the group consisting of 4-(3-hydroxy-phenyl), 4-(indol-5-yl), 4-(indol-6-yl), 4-(isoquinolin-6-yl), 4-(indazol-4-yl), 4-(1-methyl-indazol-5-yl), and 4-(benzofuran-5-yl) and 4-(benzthiazol-5-yl); and a stereoisomer, a tautomer, and a pharmaceutically acceptable salt thereof.

In another embodiment, R¹ and R² are taken together to form a ring structure selected from the group consisting of cyclopropyl and cyclopentyl; m is an integer from 0 to 1; and n is 0;

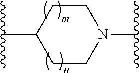

is selected from the group consisting of azetidin-3-yl and pyrrolidin-3R-yl; a is 1; L¹ is-C(O)—; R³ is selected from the group consisting of cyclopropyl, 1-hydroxy-cyclopropyl, 1-methyl-cyclopropyl and oxetan-2-yl;

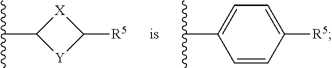

b is an integer from 0 to 1; R⁴ is selected from the group consisting of 2-fluoro and 2-methyl; R⁵ is selected from the group consisting of (a)

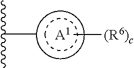

and (b)

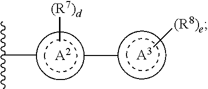

wherein

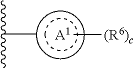

is selected from the group consisting of naphtha-2-yl, 6-chloro-naphth-2-yl, 6-fluoro-naphth-2-yl, 6-methyl-naphth-2-yl, 6-methoxy-naphth-2-yl, 6-cyano-naphth-2-yl, indol-5-yl, indol-6-yl, 1-methyl-indol-5-yl, 2-methyl-indol-5-yl, 2-hydroxymethyl-indol-5-yl, 3-(2-hydroxyethyl)-indol-5-yl, 3-cyanomethyl-indol-5-yl, indazol-5-yl, indazol-6-yl, 1-methyl-indazol-5-yl, quinolin-7-yl, 3-chloro-quinolin-7-yl, 6-fluoro-quinolin-2-yl, 8-fluoro-quinolin-2-yl, isoquinolin-6-yl, benzofuran-5-yl, 2-methyl-benzofuran-5-yl, 2-cyano-benzofuran-5-yl, benzothien-5-yl, 2-methyl-benzothien-5-yl, 2,3-dimethyl-benzothien-5-yl, benzoxazol-2-yl, benzothiazol-2-yl and 1-methyl-benzimidazol-5-yl; wherein

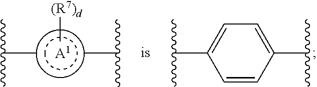

and wherein

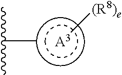

is selected from the group consisting of pyridine-4-yl and 1-methyl-pyrazol-4-yl; and a stereoisomer, a tautomer, and a pharmaceutically acceptable salt thereof.

In another embodiment, R¹ and R² are taken together to form a ring structure selected from the group consisting of cyclopropyl, cyclopentyl, 1-(methyl)-piperidin-4,4-diyl, 1-(methoxy-carbonyl)-piperidin-4 4-diyl and 1-(benzyl)-piperidin-4,4-diyl; m is an integer from 0 to 1; n is 0;

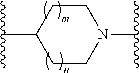

is selected from the group consisting of azetidin-3-yl and pyrrolidin-3R-yl; a is 1; L¹ is —C(O)—; R³ is cyclopropyl;

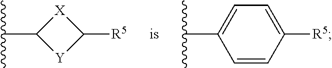

b is an integer from 0 to 1; R⁴ is 2-methyl; R⁵ is

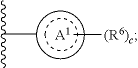

wherein

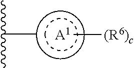

is selected from the group consisting of 4-(4-cyano-phenyl), 4-(3-hydroxy-phenyl), 4-(3-chloro-phenyl), 4-(4-chloro-phenyl), 4-(4-methyl-phenyl), 4-(4-methoxy-phenyl), 4-(indol-4-yl), 4-(indol-5-yl), 4-(indol-6-yl), 4-(quinolin-5-yl), 4-(isoquinolin-6-yl), 4-(isoquinolin-7-yl), 4-(indazol-4-yl), 4-(indazol-5-yl), 4-(1-methyl-indazol-5-yl), 4-(1-methyl-indazol-6-yl), 4-(benzofuran-5-yl) and 4-(benzthiazol-5-yl); and a stereoisomer, a tautomer, and a pharmaceutically acceptable salt thereof.

In another embodiment, R¹ and R² are taken together to form a ring structure selected from the group consisting of cyclopropyl, cyclopentyl and tetrahydropyran-4,4-diyl; m is an integer from 0 to 1; n is 0;

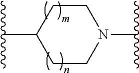

is selected from the group consisting of azetidin-3-yl and pyrrolidin-3R-yl; a is 1; L¹ is —C(O)—; R³ is selected from the group consisting of cyclopropyl, 1-fluoro-cyclopropyl, 1-hydroxy-cyclopropyl, 1-methyl-cyclopropyl, tetrahydrofuran-2-yl, tetrahydrofuran-2S-yl and oxetan-2-yl;

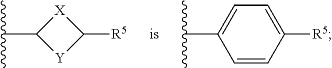

b is an integer from 0 to 1; R⁴ is selected from the group consisting of 2-fluoro and 2-methyl; R⁵ is selected from the group consisting of (a)

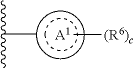

and (b)

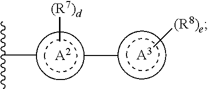

wherein

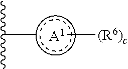

is selected from the group consisting of naphth-2-yl, 6-chloro-naphth-2-yl, 6-fluoro-naphth-2-yl, 7-fluoro-naphth-2-yl, 8-fluoro-naphth-2-yl, 6-methyl-naphth-2-yl, 6-methoxy-naphth-2-yl, 6-cyano-naphth-2-yl, indol-3-yl, indol-5-yl, indol-6-yl, 1-methyl-indol-5-yl, 2-methyl-indol-6-yl, 3-(2-hydroxyethyl)-indol-5-yl, 3-cyanomethyl-indol-5-yl, 1,3-dimethyl-indol-5-yl, 1-methyl-3-(2-hydroxyethyl)-indol-5-yl, quinolin-7-yl, 3-chloro-quinolin-7-yl, 6-fluoro-quinolin-6-yl, isoquinolin-6-yl, quinazolin-7-yl, indazol-4-yl, indazol-5-yl, indazol-6-yl, 1-methyl-indazol-5-yl, 2-methyl-indazol-6-yl, 1-methyl-3-amino-indazol-6-yl, 1-methyl-3-aminocarbonyl-indazol-6-yl, benzofuran-5-yl, 2-methyl-benzofuran-5-yl, 2-methyl-benzothien-5-yl, benzothiazol-5-yl, 6-chloro-benzothiazol-2-yl, 6-methyl-benzothiazol-2-yl, 6-cyano-benzothiazol-2-yl, benzimidazol-5-yl and 1-methyl-benzimidazol-5-yl; wherein

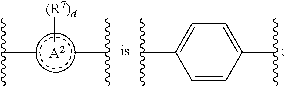

and wherein

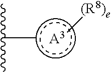

is selected from the group consisting of 1-methyl-pyrazol-4-yl, 1-isopropyl-pyrazol-4-yl, 1-cyclopropyl-pyrazol-4-yl, 1-cyclobutyl-pyrazol-4-yl, 1-methyl-pyrazol-5-yl and pyridin-4-yl; and a stereoisomer, a tautomer, and a pharmaceutically acceptable salt thereof.

In another embodiment, R¹ and R² are taken together to form a ring structure selected from the group consisting of cyclopropyl and cyclopentyl; m is an integer from 0 to 1; and n is 0;

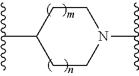

is selected from the group consisting of azetidin-3-yl and pyrrolidin-3R-yl; a is 1; L¹ is-C(O)—; R³ is selected from the group consisting of cyclopropyl, 1-fluoro-cyclopropyl, 1-hydroxy-cyclopropyl, 1-methyl-cyclopropyl, 1-methyl-cyclobutyl, tetrahydrofuran-2-yl; tetrahydrofuran-2S-yl and oxetan-2-yl;

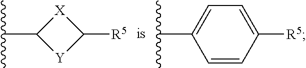

b is an integer from 0 to 1; R⁴ is selected from the group consisting of 2-fluoro, 2-chloro and 2-methyl; R⁵ is selected from the group consisting of (a)

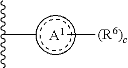

and (b)

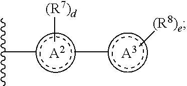

wherein

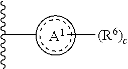

is selected from the group consisting of 3-(cyclopropyl-sulfonylamino)-phenyl, naphth-2-yl, 6-chloro-naphth-2-yl, 6-fluoro-naphth-2-yl, 7-fluoro-naphth-2-yl, 8-fluoro-naphth-2-yl, 6-methyl-naphth-2-yl, 6-methoxy-naphth-2-yl, 6-cyano-naphth-2-yl, indol-3-yl, indol-5-yl, indol-6-yl, 1-methyl-indol-5-yl, 2-hydroxymethyl-indol-5-yl, 2-methyl-indol-5-yl, 3-(2-hydroxyethyl)-indol-5-yl, quinolin-7-yl, 3-chloro-quinolin-7-yl, 8-fluoro-quinolin-2-yl, quinazolin-7-yl, indazol-4-yl, indazol-5-yl, indazol-6-yl, 1-methyl-indazol-5-yl, 2-methyl-indazol-6-yl, 2-methyl-benzothien-5-yl, 6-chloro-benzothiazol-2-yl, 6-methyl-benzothiazol-2-yl, benzoxazol-2-yl, benzimidazol-5-yl and 1-methyl-benzimidazol-5-yl; wherein

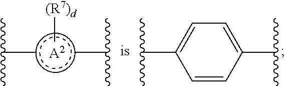

and wherein

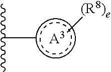

is selected from the group consisting of 1-methyl-pyrazol-4-yl, 1-isopropyl-pyrazol-4-yl, 1-cyclopropyl-pyrazol-4-yl and 1-cyclobutyl-pyrazol-4-yl; and a stereoisomer, a tautomer, and a pharmaceutically acceptable salt thereof.

In another embodiment, R¹ and R² are taken together to form a cyclopropyl; m is an integer from 0 to 1; and n is 0;

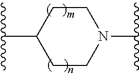

is selected from the group consisting of azetidin-3-yl and pyrrolidin-3R-yl; a is 1; L¹ is-C(O)—; R³ is cyclopropyl;

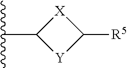

is selected from the group consisting of

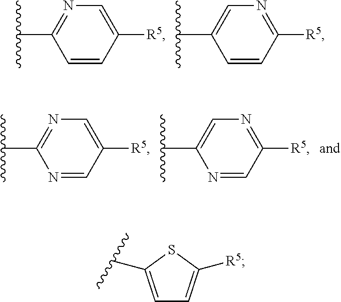

### b is 0; R⁵ is

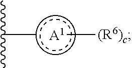

wherein

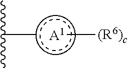

is selected from the group consisting of indol-5-yl, indazol-4-yl, indazol-5-yl, 1-methyl-indazol-5-yl, benzthiazol-5-yl, benzofuran-5-yl, benzothien-5-yl and 6-cyano-naphth-2-yl; and a stereoisomer, a tautomer, and a pharmaceutically acceptable salt thereof.

In another embodiment, R¹ and R² are taken together to form a ring structure selected from the group consisting of cyclopropyl, cyclopentyl, tetrahydro-furan-3,3-diyl, tetrahydro-pyran-4,4-diyl, 1-(methoxycarbonyl)-azetidin-3,3-diyl, piperidin-4,4-diyl, 1-(isopropylcarbonyl)-piperidin-4,4-diyl, 1-(2-hydroxyethyl)-piperidin-4,4-diyl, 1-(dimethylamino-methylcarbonyl)-piperidin-4,4-diyl, 1-(methylsulfonyl) piperidin-4,4-diyl and 1-(cyclopropylcarbonyl)-piperidin-4,4-diyl; m is an integer from 0 to 2; and n is an integer from 0 to 1; provided that when m is 2 then n is 0;

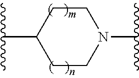

is selected from the group consisting of azetidin-3-yl, pyrrolidin-3R-yl, pyrrolidin-3R-yl, piperidin-3R-yl, and piperidin-4-yl; a is 1; L¹ is selected from the group consisting of —C(O)—, —C(O)O-and-SO₂—; R³ is selected from the group consisting of methyl, 1-hydroxyethyl, trifluoromethyl, cyclopropyl, 1-methyl-cyclopropyl, 1-hydroxy-cyclopropyl, tetrahydro-furan-2R-yl, pyrrolidin-1-yl and thiazol-2-yl;

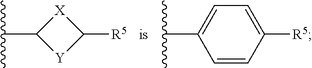

b is a integer from 0 to 1; R⁴ is selected from the group consisting of 2-fluoro and 2-methyl; R⁵ is

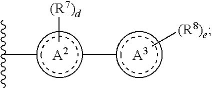

wherein

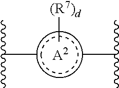

is selected from the group consisting of phenyl, pyridin-3-yl, pyridin-4-yl and pyrazol-4-yl; and wherein

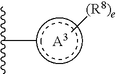

is selected from the group consisting of 4-bromo-phenyl, 3-chloro-phenyl, 4-methyl-phenyl, pyridin-3-yl, pyridin-4-yl, 1-methyl-pyrazol-3-yl, 1-(cyclopropylmethyl)-pyrazol-3-yl, 1-(2-methylpropyl)-pyrazol-3-yl, 1-methyl-pyrazol-4-yl, 1-isopropyl-pyrazol-4-yl, 1-cyclopropyl-pyrazol-4-yl, 1-cyclobutyl-pyrazol-4-yl, 1-methyl-pyrazol-5-yl, 1-isobutyl-pyrazol-5-yl, 1-(cyclopropylmethyl)-pyrazol-5-yl, tetrazol-5-yl, 5-methyl-oxadiazol-2-yl, piperazin-1-yl, 4-methyl-piperazin-1-yl, pyrrolidin-1-yl, morpholin-14-yl, imidazol-1-yl and oxetan-3-yl; provided that when

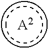

is a phenyl or pyridine-3-yl, then

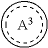

is bound to

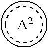

at the 4-position, relative to the point of attachment of the

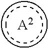

to the

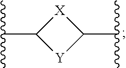

provided further that when

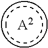

is a pyridine-4-yl or pyrazol-4-yl, then

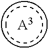

is bound to

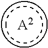

at the 3-position, relative to the point of attachment of the

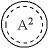

to the

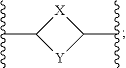

and a stereoisomer, a tautomer, and a pharmaceutically acceptable salt thereof.

In some embodiments, R¹ and R² are taken together to form a ring structure selected from the group consisting of cyclopropyl and cyclopentyl; n is an integer from 0 to 1; m is an integer from 0 to 1;

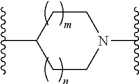

is selected from the group consisting of azetidin-3-yl, pyrrolidin-3R-yl, and piperidin-4-yl; a is 1; L¹ is —C(O)—; R³ is cyclopropyl;

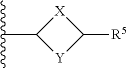

is phenyl; R⁵ is

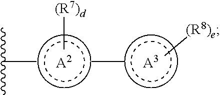

wherein

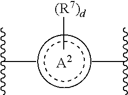

is 4-(phenyl); and wherein

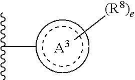

is selected from the group consisting of 4-(4-bromo-phenyl), 4-(pyridin-3-yl), 4-(pyridin-4-yl), 4-(1-methyl-pyrazol-4-yl), 4-(1-methyl-pyrazol-5-yl), 4-(tetrazol-5-yl), and 3-(pyrazol-3-yl); and a stereoisomer, a tautomer, and a pharmaceutically acceptable salt thereof.

In some embodiments, R¹ and R² are taken together to form a ring structure selected from the group consisting of cyclopropyl and cyclopentyl; m is an integer from 0 to 1; and n is 0;

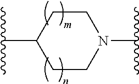

is selected from the group consisting of azetidin-3-yl and pyrrolidin-3R-yl; a is 1; L¹ is-C(O)—; R³ is selected from the group consisting of cyclopropyl, 1-hydroxy-cyclopropyl and 1-methyl-cyclopropyl;

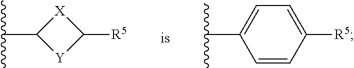

b is an integer from 0 to 1; R⁴ is selected from the group consisting of 2-fluoro and 2-methyl; R⁵ is

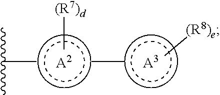

wherein

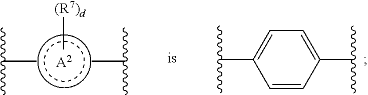

and wherein

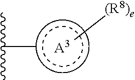

is selected from the group consisting of 4-(pyridin-3-yl), 4-(pyridin-4-yl), 4-(1-methyl-pyrazol-4-yl), 4-(1-isopropyl-pyrazol-4-yl), 4-(1-cyclopropyl-pyrazol-4-yl), 4-(1-cyclobutyl-pyrazol-4-yl), 4-(1-methyl-pyrazol-5-yl), and 4-(5-methyl-oxadiazol-2-yl); wherein

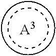

is bound to the

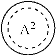

phenyl at the 4-position, relative to the point of attachment of the

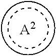

phenyl to the

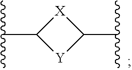

and a stereoisomer, a tautomer, and a pharmaceutically acceptable salt thereof.

In some embodiments, R¹ and R² are taken together to form cyclopropyl, m is an integer from 0 to 1; n is 0;

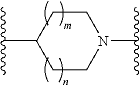

is selected from the group consisting of azetidin-3-yl and pyrrolidin-3R-yl; a is 1; L¹ is-C(O)—; R³ is cyclopropyl,

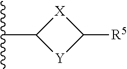

is phenyl; R⁵ is

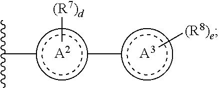

wherein

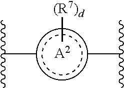

is 4-(phenyl); and wherein

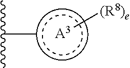

is selected from the group consisting of 4-(pyridin-3-yl) and 4-(1-methyl-pyrazol-4-yl); and a stereoisomer, a tautomer, and a pharmaceutically acceptable salt thereof.

In a preferred embodiment, the present invention is directed to compounds of formula (XXVIII) wherein R¹ and R² are taken together to form an optionally substituted ring structure selected from the group consisting of (a) C₃₋₆cycloalkyl; wherein the C₃₋₈cycloalkyl is optionally substituted with one R¹¹ group; (b) benzo-fused C₅₋₆cycloalkyl; wherein the benzo-fused C₅₋₆cycloalkyl is bound through a carbon atom of the C₅₋₆cycloalkyl portion of the ring structure; and wherein the benzo-fused C₅₋₆cycloalkyl is optionally substituted with one R¹¹ group; and (c) 4 to 8 membered, saturated heterocyclyl; wherein the 4 to 8 membered, saturated heterocyclyl contains O or NR¹⁰; provided that the O or NR¹⁰ is not present at the 2-position relative to the carbon atom of the imidazolin-5-one; and wherein the 4 to 8 membered, saturated heterocyclyl containing the O or NR¹⁰ is optionally substituted with one R¹¹ group and further optionally substituted with one R¹² group.

In another preferred embodiment, the present invention is directed to compounds of formula (XXVIII) wherein R¹ and R² are taken together to form an optionally substituted ring structure selected from the group consisting of (a) C₃₋₆cycloalkyl; and (c) 4 to 6 membered, saturated heterocyclyl; wherein the 4 to 6 membered saturated heterocyclyl contains NR¹⁰; provided that the NR¹⁰ is not present at the 2-position relative to the carbon atom of the imidazolidin-5-one.

In another preferred embodiment, the present invention is directed to compounds of formula (XXVIII) wherein R¹ and R² are taken together to form an optionally substituted ring structure selected from the group consisting of (a) C₃₋₆cycloalkyl; wherein the C₃₋₈cycloalkyl is optionally substituted with one R¹¹ group; (b) benzo-fused C₅₋₆cycloalkyl; wherein the benzo-fused C₅₋₆cycloalkyl is bound through a carbon atom of the C₅₋₆cycloalkyl portion of the ring structure; and wherein the benzo-fused C₅₋₆cycloalkyl is optionally substituted with one R¹¹ group; and (c) 4 to 6 membered, saturated heterocyclyl; wherein the 4 to 6 membered, saturated heterocyclyl contains O or NR¹⁰; provided that the O or NR¹⁰ is not present at the 2-position relative to the carbon atom of the imidazolin-5-one; and wherein the 4 to 6 membered, saturated heterocyclyl containing the O or NR¹⁰ is optionally substituted with one R¹¹ group and further optionally substituted with one R¹².

In another preferred embodiment, the present invention is directed to compounds of formula (XXVIII) wherein R¹ and R² are taken together to form an optionally substituted ring structure selected from the group consisting of (a) C₃₋₆cycloalkyl; and (c) 4 to 6 membered, saturated heterocyclyl; wherein the 4 to 6 membered saturated heterocyclyl contains NR¹⁰; provided that the NR¹⁰ is not present at the 2-position relative to the carbon atom of the imidazolidin-5-one.

In another preferred embodiment, the present invention is directed to compounds of formula (XXVIII) wherein R¹ and R² are taken together to form a ring structure selected from the group consisting of cyclopropyl, cyclopentyl, piperidin-4,4-diyl, 1-(methyl)-piperidin-4,4-diyl, 1-(isopropyl)-piperidin-4,4-diyl, 1-(ethenyl)-piperidin-4,4-diyl, 1-(2-hydroxy-ethyl)-piperidin-4,4-diyl, 1-(methoxy-carbonyl)-piperidin-4,4-diyl, 1-(benzyl)-piperidin-4,4-diyl, 1-(methyl-carbonyl)-piperidin-4,4-diyl, 1-(isopropyl-carbonyl)-piperidin-4,4-diyl, 1-(trifluoromethyl-carbonyl)-piperidin-4,4-diyl, 1-(cyclopropyl-carbonyl)-piperidin-4,4-diyl, 1-(dimethylamino-carbonyl)-piperidin-4,4-diyl, 1-(methyl-sulfonyl)-piperidin-4,4-diyl, 1-(2-methoxy-ethyl)-piperidin-4,4-diyl, 1-(benzyl)-piperidin-4,4-diyl, tetrahydro-pyran-4,4-diyl, tetrahydro-furan-3,3-diyl, and 1-(methoxycarbonyl)-azetidin-3,3-diyl.

In another preferred embodiment, the present invention is directed to compounds of formula (XXVIII) wherein R¹ and R² are taken together to form a ring structure selected from the group consisting of cyclopropyl, cyclopentyl, piperidin-4,4-diyl, 1-(methyl)-piperidin-4,4-diyl, 1-(isopropyl)-piperidin-4,4-diyl, 1-(2-hydroxy-ethyl)-piperidin-4,4-diyl, 1-(ethenylcarbonyl)-piperidin-4,4-diyl, 1-(trifluoromethyl-carbonyl)piperidin-4,4-diyl, 1-(methoxy-carbonyl)-piperidin-4,4-diyl, 1-(2-methoxyethyl)-piperidin-4,4-diyl, 1-(benzyl)-piperidin-4,4-diyl, 1-(methyl-carbonyl)-piperidin-4,4-diyl, 1-(isopropyl-carbonyl)-piperidin-4,4-diyl, 1-(cyclopropyl-carbonyl)-piperidin-4,4-diyl, 1-(methylsulfonyl)-piperidin-4,4-diyl, 1-(dimethylamino-carbonyl)-piperidin-4,4-diyl, 1-(methoxycarbonyl)-azetidin-3,3-diyl, tetrahydrofuran-3,3-diyl, and tetrahydro-pyran-4,4-diyl.

In another preferred embodiment, the present invention is directed to compounds of formula (XXVIII) wherein R¹ and R² are taken together to form a ring structure selected from the group consisting of cyclopropyl, cyclopentyl, piperidin-4,4-diyl, 1-(methyl)-piperidin-4,4-diyl, 1-(isopropyl)-piperidin-4,4-diyl, 1-(2-hydroxy-ethyl)-piperidin-4,4-diyl, 1-(methoxy-carbonyl)-piperidin-4,4-diyl, 1-(benzyl)-piperidin-4,4-diyl, 1-(methyl-carbonyl)-piperidin-4,4-diyl, 1-(isopropyl-carbonyl)-piperidin-4,4-diyl, 1-(cyclopropyl-carbonyl)-piperidin-4,4-diyl, 1-(dimethylamino-carbonyl)-piperidin-4,4-diyl, 1-(trifluoromethyl-carbonyl)-piperidin-4,4-diyl, 1-(methyl-sulfonyl)-piperidin-4,4-diyl, 1-(2-methoxyethyl)-piperidin-4,4-diyl, 1-(methoxycarbonyl)azetidin-3,3-diyl, tetrahydro-furan-3,3-diyl, and tetrahydro-pyran-4,4-diyl.

In another preferred embodiment, the present invention is directed to compounds of formula (XXVIII) wherein R¹ and R² are taken together to form a ring structure selected from the group consisting of cyclopropyl, cyclopentyl, tetrahydro-furan-3,3-diyl, tetrahydro-pyran-4,4-diyl, 1-(methoxycarbonyl)-azetidin-3,3-diyl, piperidin-4,4-diyl, 1-(isopropylcarbonyl)-piperidin-4,4-diyl, 1-(2-hydroxyethyl)-piperidin-4,4-diyl, 1-(dimethylamino-methylcarbonyl)-piperidin-4,4-diyl, 1-(methylsulfonyl)piperidin-4,4-diyl, and 1-(cyclopropylcarbonyl)-piperidin-4,4-diyl.

In another preferred embodiment, the present invention is directed to compounds of formula (XXVIII) wherein R¹ and R² are taken together to form a ring structure selected from the group consisting of cyclopropyl, cyclopentyl, piperidin-4,4-diyl, 1-(methyl)-piperidin-4,4-diyl, 1-(isopropyl)-piperidin-4,4-diyl, 1-(2-hydroxy-ethyl)-piperidin-4,4-diyl, 1-(methoxy-carbonyl)-piperidin-4,4-diyl, 1-(benzyl)-piperidin-4,4-diyl, 1-(methyl-carbonyl)-piperidin-4,4-diyl, 1-(isopropyl-carbonyl)-piperidin-4,4-diyl, 1-(cyclopropyl-carbonyl)-piperidin-4,4-diyl, and 1-(dimethylamino-carbonyl)-piperidin-4,4-diyl.

In another preferred embodiment, the present invention is directed to compounds of formula (XXVIII) wherein R¹ and R² are taken together to form a ring structure selected from the group consisting of cyclopropyl, cyclopentyl, piperidin-4,4-diyl, 1-(methyl)-piperidin-4,4-diyl, 1-(2-hydroxy-ethyl)-piperidin-4,4-diyl, 1-(methoxy-carbonyl)-piperidin-4,4-diyl, 1-(benzyl)-piperidin-4,4-diyl, 1-(methyl-carbonyl)-piperidin-4,4-diyl, 1-(isopropyl-carbonyl)-piperidin-4,4-diyl, 1-(cyclopropyl-carbonyl)-piperidin-4,4-diyl, and 1-(dimethylamino-carbonyl)-piperidin-4,4-diyl.

In another preferred embodiment, the present invention is directed to compounds of formula (XXVIII) wherein R¹ and R² are taken together to form a ring structure selected from the group consisting of cyclopropyl, cyclopentyl, 1-(methoxy-carbonyl)-piperidin-4,4-diyl, 1-(isopropyl-carbonyl)-piperidin-4,4-diyl, and 1-(dimethylamino-carbonyl)-piperidin-4,4-diyl.

In another preferred embodiment, the present invention is directed to compounds of formula (XXVIII) wherein R¹ and R² are taken together to form a ring structure selected from the group consisting of cyclopropyl, cyclopentyl, 1-(methyl)-piperidin-4,4-diyl, 1-(methoxy-carbonyl)-piperidin-4,4-diyl, and 1-(benzyl)-piperidin-4,4-diyl.

In another preferred embodiment, the present invention is directed to compounds of formula (XXVIII) wherein R¹ and R² are taken together to form a ring structure selected from the group consisting of cyclopropyl, cyclopentyl, and tetrahydropyran-4,4-diyl.

In another preferred embodiment, the present invention is directed to compounds of formula (XXVIII) wherein R¹ and R² are taken together to form a ring structure selected from the group consisting of cyclopropyl and cyclopentyl. In another preferred embodiment, the present invention is directed to compounds of formula (XXVIII) wherein R¹ and R² are taken together to form cyclopropyl.

In another embodiment, R¹⁰ is selected from the group consisting of hydrogen, C₁₋₄alkyl, fluorinated C₁₋₄alkyl, —CH₂-(hydroxy substituted C₁₋₄alkyl), —(C₁₋₄alkyl)-phenyl, —C(O)—NR^{A}R^{B}, —C(O)—(C₁₋₄alkyl), —C(O)—(C₃₋₆cycloalkyl),

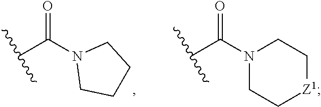

wherein Z¹ is selected from the group consisting of —CH₂—, —O—, and —NR_{c}—; and wherein R^{A}, R^{B} and R^{C} are each independently selected from the group consisting of hydrogen and C₁₋₄alkyl;

In another preferred embodiment, the present invention is directed to compounds of formula (XXVIII) wherein R¹⁰ is selected from the group consisting of hydrogen, C₁₋₄alkyl, —CH₂-(hydroxy substituted C₁₋₂alkyl), —CH₂-(phenyl), —C(O)—(C₁₋₄alkyl), —C(O)-(cyclopropyl) and —C(O)—NR^{A}R^{B}; wherein R^{A} and R^{B} are each independently selected from the group consisting of hydrogen and methyl.

In another embodiment, R¹⁰ is selected from the group consisting of hydrogen, C₁₋₄alkyl, fluorinated C₁₋₄alkyl, —CH₂-(hydroxy substituted C₁₋₄alkyl), —(C₂₋₄alkenyl), —(C₁₋₄alkyl)-phenyl, -(C₂alkyl)-O—(C₁₋₄alkyl), —C(O)O—(C₁₋₄alkyl), —C(O)—(C₁₋₄alkyl), —C(O)-(fluorinated C₁₋₂alkyl), —C(O)—(C₃₋₆cycloalkyl),

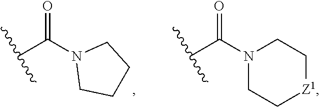

—C(O)—NR^{A}R^{B}, —SO₂—(C₁₋₂alkyl); Z¹ is selected from the group consisting of —CH₂—, —O—, and —NR_{c}—; and wherein R^{A}, R^{B} and R^{C} are each independently selected from the group consisting of hydrogen and C₁₋₄alkyl.

In another preferred embodiment, the present invention is directed to compounds of formula (XXVIII) wherein R¹⁰ is selected from the group consisting of hydrogen, C₁₋₄alkyl, C₂₋₄alkenyl, —CH₂-(hydroxy substituted C₁₋₂alkyl), —CH₂-(phenyl), —(C₂alkyl)-O—(C₁₋₂alkyl), —C(O)—(C₁₋₄alkyl), —C(O)-(fluorinated C₁₋₂alkyl), —C(O)-(cyclopropyl), —C(O)O—(C₁₋₄alkyl), —C(O)—NR^{A}R^{B}, —SO₂—(C₁₋₂alkyl), wherein R and R are each independently selected from the group consisting of hydrogen and methyl.

In a preferred embodiment, the present invention is directed to compounds of formula (XXVIII) wherein R¹¹ is independently selected from the group consisting of hydroxy, oxo, halogen, C₁₋₄alkyl, fluorinated C₁₋₄alkyl, C₁₋₄alkoxy, fluorinated C₁₋₄alkoxy, hydroxy substituted C₁₋₄alkyl, —(C₁₋₄alkyl)-phenyl, cyano, —NR^{D}R^{E}, —C(O)—NR^{D}R^{E}, —C(O)—(C₁₋₄alkyl), —C(O)OH and —C(O)O—(C₁₋₄alkyl); wherein R¹² is selected from the group consisting of hydroxy, oxo, halogen, C₁₋₂alkyl, CF₃, C₁₋₂alkoxy, —OCF₃ and hydroxy substituted C₁₋₂alkyl.

In another preferred embodiment, the present invention is directed to compounds of formula (XXVIII) wherein R¹¹ is independently selected from the group consisting of hydroxy, oxo, halogen, C₁₋₄ alkyl, fluorinated C₁₋₄alkyl, C₁₋₄alkoxy, fluorinated C₁₋₄alkoxy, hydroxy substituted C₁₋₄alkyl, —(C₁₋₄alkyl)-phenyl, -cyano, —NR^{D}R^{E}, —C(O)—NR^{D}R^{E}, —C(O)—(C₁₋₄alkyl), —C(O)OH and —C(O)O—(C₁₋₄alkyl).

In a preferred embodiment, the present invention is directed to compounds of formula (XXVIII) wherein R¹² is selected from the group consisting of hydroxy, oxo, halogen, C₁₋₂alkyl, CF₃, C₁₋₂alkoxy, OCF₃ and hydroxy substituted C₁₋₂alkyl. In another preferred embodiment, the present invention is directed to compounds of formula (XXVIII) wherein R¹² is selected from the group consisting of —OH, oxo, —C₁, —F, —CH₃, CF₃, —OCH₃, —OCF₃, —CH₂—OH and —CH₂CH₂—OH.

In a preferred embodiment, the present invention is directed to compounds of formula (XXVIII) wherein m is an integer from 0 to 1; and n is an integer from 0 to 2; provided that when n is 2, then m is 0. In another preferred embodiment, the present invention is directed to compounds of formula (XXVIII) wherein m is 0. In another preferred embodiment, the present invention is directed to compounds of formula (XXVIII) wherein m is 1. In another preferred embodiment, the present invention is directed to compounds of formula (XXVIII) wherein n is 0. In another preferred embodiment, the present invention is directed to compounds of formula (XXVIII) wherein n is 1. In another preferred embodiment, the present invention is directed to compounds of formula (XXVIII) wherein n is 2. In a preferred embodiment, the present invention is directed to compounds of formula (XXVIII) wherein m is 0 and n is 0. In a preferred embodiment, the present invention is directed to compounds of formula (XXVIII) wherein m is 1 and n is 1. In a preferred embodiment, the present invention is directed to compounds of formula (XXVIII) wherein m is 1 and n is 0 or alternatively, m is 0 and n is 1. In another preferred embodiment, the present invention is directed to compounds of formula (XXVIII) wherein m is 0 and n is 2.

In another embodiment,

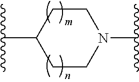

is selected from the group consisting of azetidin-3-yl, pyrrolidin-3-yl, pyrrolidin-3R-yl, pyrrolidin-3S-yl, piperidin-3-yl, piperidin-3R-yl, piperidin-3S-yl, and piperidin-4-yl. In another embodiment

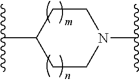

is selected from the group consisting of azetidin-3-yl, pyrrolidin-3-yl, pyrrolidin-3R-yl, pyrrolidin-3S-yl, and piperidin-4-yl. In another embodiment,

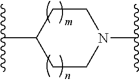

is selected from the group consisting of azetidin-3-yl, pyrrolidin-3R-yl, pyrrolidin-3S-yl, and piperidin-4-yl. In another embodiment,

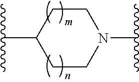

is selected from the group consisting of azetidin-3-yl, pyrrolidin-3R-yl, and piperidin-4-yl. In another embodiment,

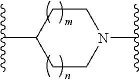

is selected from the group consisting of azetidin-3-yl and pyrrolidin-3R-yl.

In a preferred embodiment, the present invention is directed to compounds of formula (XXVIII) wherein a is 1. In another preferred embodiment, the present invention is directed to compounds of formula (XXVIII) wherein a is 0.

In a preferred embodiment, the present invention is directed to compounds of formula (XXVIII) wherein L¹ is selected from the group consisting of —C(O)—, —C(O)O—, —C(O)—NR^{L}-and-SO₂—; wherein R^{L} is selected from the group consisting of hydrogen and methyl. In another preferred embodiment, the present invention is directed to compounds of formula (XXVIII) wherein L¹ is selected from the group consisting of —C(O)—, —C(O)O— and —SO₂—.

In another preferred embodiment, the present invention is directed to compounds of formula (XXVIII) wherein L¹ is selected from the group consisting of —C(O)—, —C(O)—NR^{L}-and-SO₂—; wherein R^{L} is selected from the group consisting of hydrogen and methyl. In another preferred embodiment, the present invention is directed to compounds of formula (XXVIII) wherein L¹ is selected from the group consisting of —C(O)-and-SO₂—. In another preferred embodiment, the present invention is directed to compounds of formula (XXVIII) wherein L¹ is-C(O)—.

In a preferred embodiment, the present invention is directed to compounds of formula (XXVIII) wherein R³ is selected from the group consisting of C₁₋₄alkyl, fluorinated C₁₋₂alkyl, hydroxy substituted C₁₋₄alkyl, C₂₋₄alkenyl, C₃₋₆cycloalkyl, 4 to 6-membered, saturated heterocyclyl, 5 to 6-membered heteroaryl and NR^{V}R^{W}; wherein R^{V} and R^{W} are each independently selected from the group consisting of hydrogen and C₁₋₂alkyl; wherein the C₃₋₆cycloalkyl, 4 to 6-membered, saturated heterocyclyl or 5 to 6-membered heteroaryl, is optionally substituted with one to two substituents independently selected from the group consisting of halogen, hydroxy, cyano, C₁₋₄alkyl, fluorinated C₁₋₄alkyl, —(C₁₋₂alkyl)-OH, C₁₋₄alkoxy, fluorinated C₁₋₄alkoxy, and NR^{G}R^{H}; wherein R^{G} and R^{H} are each independently selected from the group consisting of hydrogen and C₁₋₄alkyl.

In another preferred embodiment, the present invention is directed to compounds of formula (XXVIII) wherein R³ is selected from the group consisting of C₁₋₄alkyl, hydroxy substituted C₁₋₄alkyl, fluorinated C₁₋₂alkyl, C₂₋₄alkenyl, C₃₋₅cycloalkyl, 4 to 5-membered, saturated heterocyclyl, 5 to 6-membered heteroaryl and NR^{V}R^{W}; wherein the C₃₋₅cycloalkyl, 4 to 5-membered, saturated heterocyclyl or 5 to 6-membered heteroaryl is optionally substituted with a substituent selected from the group consisting of halogen, hydroxy, (C₁₋₂alkyl)-OH, fluorinated cyano and NH₂; and wherein R^{V} and R^{W} are each independently selected from the group consisting of hydrogen and methyl.

In another preferred embodiment, the present invention is directed to compounds of formula (XXVIII) wherein R³ is selected from the group consisting of C₂₋₄alkenyl, C₃₋₆cycloalkyl, 5 to 6-membered, saturated heterocyclyl and 5 to 6-membered heteroaryl; wherein the C₃₋₆cycloalkyl, 5 to 6-membered, saturated heterocyclyl or 5 to 6-membered heteroaryl, is optionally substituted with one to two substituents independently selected from the group consisting of halogen, hydroxy, cyano, C₁₋₄alkyl, fluorinated C₁₋₄alkyl, C₁₋₄alkoxy, fluorinated C₁₋₄alkoxy and NR^{G}R^{H}; wherein R^{G} and R^{H} are each independently selected from the group consisting of hydrogen and C₁₋₄alkyl. In another preferred embodiment, the present invention is directed to compounds of formula (XXVIII) wherein R³ is selected from the group consisting of C₂alkenyl, C₃cycloalkyl, 5-membered, saturated heterocyclyl and 5-membered heteroaryl; wherein the C₃cycloalkyl, 5-membered, saturated heterocyclyl or 5-membered heteroaryl is optionally substituted with a substituent selected from the group consisting of halogen, C₁₋₂ alkyl, fluorinated C₁₋₂alkyl and cyano.

In another preferred embodiment, the present invention is directed to compounds of formula (XXVIII) wherein R³ is selected from the group consisting of methyl, ethyl, isopropyl, 1-hydroxyethyl, trifluoromethyl, 2,2,2-trifluoroethyl, 2-hydroxy-propan-2-yl. 3-hydroxy-2-methyl-propan-2-yl, ethenyl, cyclopropyl, 1-fluoro-cyclopropyl, 1-hydroxy-cyclopropyl, 1-hydroxymethyl-cyclopropyl, 1-methyl-cyclopropyl, 1-cyano-cyclopropyl, 1-amino-cyclopropyl, cyclobutyl, 1-methyl-cyclobutyl, amino, dimethylamino, pyrrolidin-1-yl, 1-methyl-pyrazol-3-yl, thiazol-2-yl, tetrahydro-furan-2-yl, tetrahydro-furan-2R-yl, oxetan-2-yl, oxetan-3-yl, 3-methyl-oxetan-3-yl, and pyridin-3-yl.

In another preferred embodiment, the present invention is directed to compounds of formula (XXVIII) wherein R³ is selected from the group consisting of ethyl, 1-hydroxy-ethyl, isopropyl, 2-hydroxy-propan-2-yl, 3-hydroxy-2-methyl-propan-2-yl, 2,2,2-trifluoroethyl, ethenyl, cyclopropyl, 1-fluoro-cyclopropyl, 1-methyl-cyclopropyl, 1-hydroxy-cyclopropyl, 1-hydroxymethyl-cyclopropyl, 1-amino-cyclopropyl, cyclobutyl, 1-methyl-cyclobutyl, pyrrolidin-1-yl, 1-methyl-pyrazol-3-yl, oxetan-2-yl, oxetan-3yl, 3-methyl-oxetan-3-yl, tetrahydro-furan-2yl, tetrahydro-furan-2R-yl, tetrahydro-furan-2S-yl and dimethylamino.

In another preferred embodiment, the present invention is directed to compounds of formula (XXVIII) wherein R³ is selected from the group consisting of 2,2,2-trifluoroethyl, ethenyl, cyclopropyl, 1-fluoro-cyclopropyl, 1-methyl-cyclopropyl, 1-cyano-cyclopropyl, pyrrolidin-1-yl, 1-methyl-pyrazol-3-yl and tetrahydro-furan-2-yl. In another preferred embodiment, the present invention is directed to compounds of formula (XXVIII) wherein R³ is selected from the group consisting of 2,2,2-trifluoroethyl, ethenyl, cyclopropyl, 1-methyl-cyclopropyl, pyrrolidin-1-yl and 1-methyl-pyrazol-3-yl.

In another preferred embodiment, the present invention is directed to compounds of formula (XXVIII) wherein R³ is selected from the group consisting of ethyl, cyclopropyl, 1-hydroxy-cyclopropyl, 1-fluoro-cyclopropyl, 1-methyl-cyclopropyl, 1-hydroxymethyl-cyclopropyl, cyclobutyl, tetrahydro-furan-2-yl, tetrahydro-furan-2R-yl, tetrahydro-furan-2S-yl, and oxetan-2-yl. In another preferred embodiment, the present invention is directed to compounds of formula (XXVIII) wherein R³ is selected from the group consisting of cyclopropyl, 1-fluoro-cyclopropyl, 1-hydroxy-cyclopropyl, 1-methyl-cyclopropyl, tetrahydrofuran-2S-yl and oxetan-2-yl. In another preferred embodiment, the present invention is directed to compounds of formula (XXVIII) wherein R³ is selected from the group consisting of cyclopropyl, 1-fluoro-cyclopropyl, 1-hydroxy-cyclopropyl, 1-methyl-cyclopropyl, tetrahydrofuran-2-yl, tetrahydrofuran-2S-yl and oxetan-2-yl. In another preferred embodiment, the present invention is directed to compounds of formula (XXVIII) wherein R³ is selected from the group consisting of methyl, 1-hydroxyethyl, trifluoromethyl, cyclopropyl, 1-methyl-cyclopropyl, 1-hydroxy-cyclopropyl, tetrahydro-furan-2R-yl, pyrrolidin-1-yl and thiazol-2-yl.

In another preferred embodiment, the present invention is directed to compounds of formula (XXVIII) wherein R³ is selected from the group consisting of cyclopropyl, 1-hydroxy-cyclopropyl, 1-methyl-cyclopropyl and oxetan-2-yl. In another preferred embodiment, the present invention is directed to compounds of formula (XXVIII) wherein R³ is selected from the group consisting of cyclopropyl, 1-hydroxy-cyclopropyl and 1-methyl-cyclopropyl. In another preferred embodiment, the present invention is directed to compounds of formula (XXVIII) wherein R³ is selected from the group consisting of cyclopropyl and 1-methyl-cyclopropyl. In another preferred embodiment, the present invention is directed to compounds of formula (XXVIII) wherein R³ is cyclopropyl.

In a preferred embodiment,

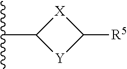

is selected from the group consisting of

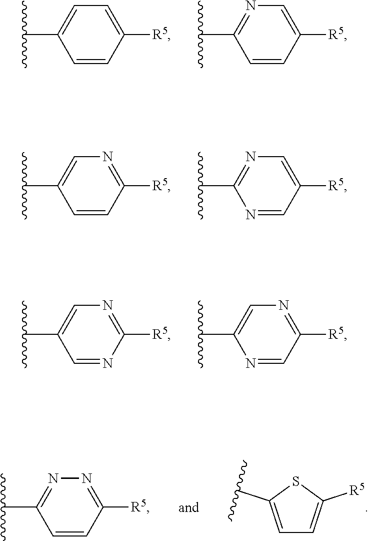

In another preferred embodiment,

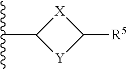

is selected from the group consisting of

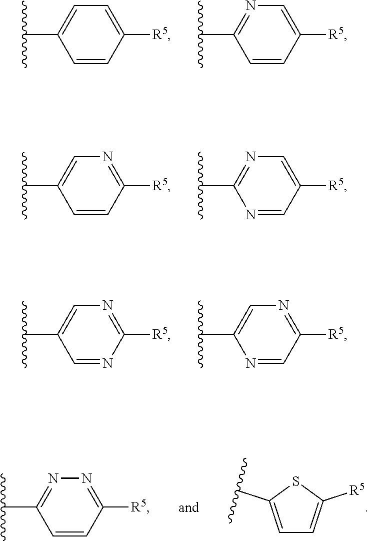

In another preferred embodiment,

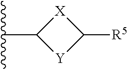

is selected from the group consisting of

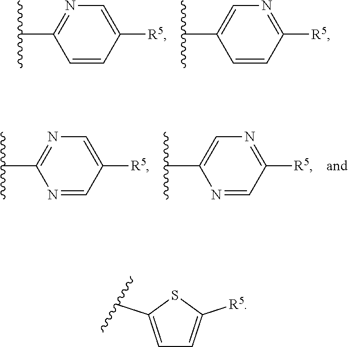

In another preferred embodiment,

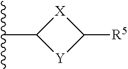

is selected from the group consisting of

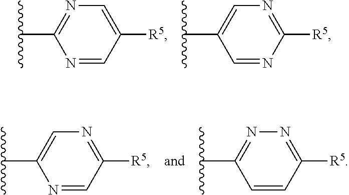

In another preferred embodiment,

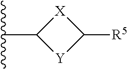

is selected from the group consisting of

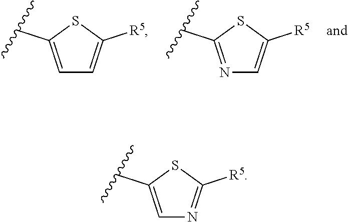

In another preferred embodiment,

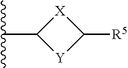

is selected from the group consisting of

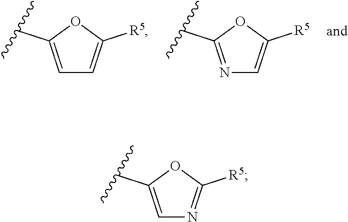

In another preferred embodiment,

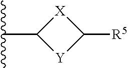

is selected from the group consisting of

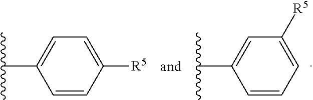

In another embodiment,

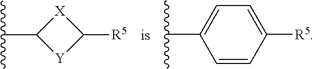

One skilled in the art will recognize that the embodiments of the present invention, as described herein, the

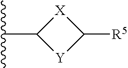

substituent group is further substituted with —(R⁴)_{b}, as defined herein.

In a preferred embodiment, the present invention is directed to compounds of formula (XXVIII) wherein b is an integer from 0 to 1. In another preferred embodiment, the present invention is directed to compounds of formula (XXVIII) wherein b is 1. In another preferred embodiment, the present invention is directed to compounds of formula (XXVIII) wherein b is 1.

In a preferred embodiment, the present invention is directed to compounds of formula (XXVIII) wherein R⁴ is selected from the group consisting of, halogen, C₁₋₄alkyl, fluorinated C₁₋₄alkyl, C₁₋₄alkoxy, fluorinated C₁₋₄alkoxy and NR^{J}R^{K}; wherein R^{J} and R^{K} are each independently selected from the group consisting of hydrogen and C₁₋₂ alkyl; provided that the R⁴ group is bound to a carbon atom. In another preferred embodiment, the present invention is directed to compounds of formula (XXVIII) wherein R⁴ is selected from the group consisting of halogen, C₁₋₂alkyl, and C₁₋₂alkoxy.

In another preferred embodiment, the present invention is directed to compounds of formula (XXVIII) wherein R⁴ is selected from the group consisting of halogen, C₁₋₂alkyl and C₁₋₂alkoxy.

In another preferred embodiment, the present invention is directed to compounds of formula (XXVIII) wherein R⁴ is selected from the group consisting of 2-fluoro, 3-fluoro, 2-chloro, 3-chloro, 2-methyl, 3-methyl and 2-methoxy. In another preferred embodiment, the present invention is directed to compounds of formula (XXVIII) wherein R⁴ is selected from the group consisting of 2-fluoro, 2-chloro, 2-methyl, 2-methoxy, 3-fluoro and 3-methyl. In another preferred embodiment, the present invention is directed to compounds of formula (XXVIII) wherein R⁴ is selected from the group consisting of 2-fluoro, 2-chloro, and 2-methyl.

In another preferred embodiment, the present invention is directed to compounds of formula (XXVIII) wherein R⁴ is selected from the group consisting of 2-fluoro, 2-methyl, 3-methyl and 2-methoxy. In another preferred embodiment, the present invention is directed to compounds of formula (XXVIII) wherein R⁴ is selected from the group consisting of 2-fluoro and 2-methyl. In another preferred embodiment, the present invention is directed to compounds of formula (XXVIII) wherein R⁴ is 2-methyl.

In a preferred embodiment, R⁵ is

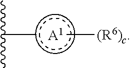

In another preferred embodiment, R⁵ is

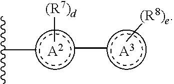

In a preferred embodiment, R⁵ is

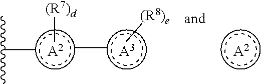

is a 5-membered heteroaryl, and

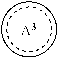

is bound at the 3-position, relative to the point of attachment of the

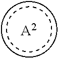

to the

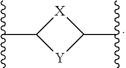

In another preferred embodiment, R⁵ is

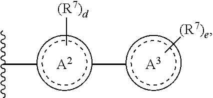

wherein

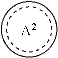

is phenyl or a 6 membered heteroaryl, and

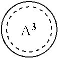

is bound at the 3- or 4-position, relative to the point of attachment of the

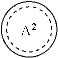

to the

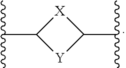

In another preferred embodiment, R⁵ is

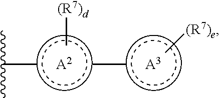

wherein

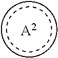

is phenyl or a 6 membered heteroaryl, and

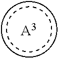

is bound at the 4-position, relative to the point of attachment of the

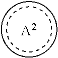

to the

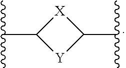

In a preferred embodiment,

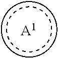

is selected from the group consisting of aryl, heteroaryl and partially unsaturated heterocyclyl. In another preferred embodiment

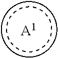

is selected from the group consisting of phenyl, naphthyl, 5 to 6 membered heteroaryl, 9 to 10 membered heteroaryl and partially unsaturated 9 to 10 membered heterocyclyl.

In another preferred embodiment,

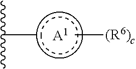

is selected from the group consisting of 3-cyano-phenyl, 4-cyano-phenyl, 3-hydroxy-phenyl, 4-hydroxy-phenyl, 3-fluoro-phenyl, 4-fluoro-phenyl, 3-chloro-phenyl, 4-chloro-phenyl, 2-fluoro-4-chloro-phenyl, 3-chloro-4-fluoro-phenyl, 2-fluoro-4-cyano-phenyl, 2-fluoro-4-(1-cyano-cyclopropyl)-phenyl, 2-fluoro-5-trifluoromethyl-phenyl, 2,4-dichloro-phenyl, 3-methyl-phenyl, 4-methyl-phenyl, 3-trifluoromethyl-phenyl, 4-trifluoromethyl-phenyl, 2-methoxy-phenyl, 3-methoxy-phenyl, 4-methoxy-phenyl, 3-trifluoromethoxy-phenyl, 4-trifluoromethoxy-phenyl, 4-(methylcarbonyl)-phenyl, 3-dimethylamino-phenyl, 4-dimethylamino-phenyl, 3-methylsulfonyl-amino-phenyl, 3-amino-4-hydroxy-phenyl, 3-formamido-4-hydroxy-phenyl 3-(cyclopropylthio)-phenyl, 3-(cyclopropylsulfonyl)-phenyl, 3-(cyclopropylcarbonyl-amino)-phenyl, 3-(cyclopropylsulfonyl-amino)-phenyl, 3-(methylsulfonyl)-phenyl, 3-(isopropylsulfonyl)-phenyl, 3-(aminocarbonyl)-phenyl, 3-carboxy-phenyl, 3-(methoxycarbonyl)-phenyl, naphth-2-yl, 6-fluoro-naphth-2-yl, 7-fluoro-naphth-2-yl, 8-fluoro-naphth-2-yl, 6-chloro-naphth-2-yl, 6-methyl-naphth-2-yl, 6-methoxy-naphth-2-yl, 8-methoxy-naphth-2-yl, 6-isopropyloxy-naphth-2-yl, 2-cyano-naphth-7-yl, 6-cyano-naphth-2-yl, 7-cyano-naphth-2-yl, 5-methoxy-naphth-2-yl, 7-methoxy-naphth-2-yl, 1,5-naphthyridin-3-yl, 1,8-naphthyridin-2-yl, 1,8-naphthyridin-3-yl, chroman-6-yl, isochroman-6-yl, isochroman-7-yl, pyridin-2-yl, pyridin-3-yl, pyridin-4-yl, 6-isopropyl-pyridin-3-yl, 6-n-propyl-pyridin-3-yl, 5-bromo-pyridin-2-yl, 5-chloro-pyridin-3-yl, 5-(2-hydroxy-2-methyl-propyl)-pyridin-2-yl, 5-(2-hydroxy-2-methyl-propyl)-pyridin-3-yl, 6-cyclopropyl-pyridin-3-yl, 6-(1-cyano-cyclopropyl)-pyridin-3-yl, 2-amino-pyrid-4-yl, 5-amino-pyridin-3-yl, 6-amino-pyridin-2-yl, 1-methyl-pyrazol-4-yl, 1-methyl-pyrazol-5-yl, indol-3-yl, indol-4-yl, indol-5-yl, indol-6-yl, 1-methyl-indol-5-yl, 1-methyl-indol-6-yl, 2-methyl-indol-5-yl, 2-hydroxymethyl-indol-5-yl, 3-(2-hydroxyethyl)-indol-5-yl, 3-cyanomethyl-indol-5-yl, 1,2-dimethyl-indol-5-yl, 1,3-dimethyl-indol-5-yl, 2,3-dimethyl-indol-5-yl, 1-methyl-3-(2-hydroxyethyl)-indol-5-yl, 1-(trifluoromethyl-carbonyl)-indol-5-yl, 2-oxo-indolin-5-yl, quinolin-2-yl, quinolin-3-yl, quinolin-5-yl, quinolin-6-yl, quinolin-7-yl, 2-chloro-quinolin-7-yl, 3-chloro-quinolin-7-yl, 4-chloro-quinolin-7-yl, 6-fluoro-quinolin-2-yl, 8-fluoro-quinolin-2-yl, 7-bromo-quinolin-2-yl, 2-hydroxy-quinolin-3-yl, 2-cyano-quinolin-6-yl, 2-cyano-quinolin-7-yl, 6-cyano-quinolin-2-yl, 2-methyl-quinolin-5-yl, 2-methyl-quinolin-6-yl, 2-methyl-quinolin-7-yl, 4-methyl-quinolin-7-yl, 2,4-dimethyl-quinolin-7-yl, 2-chloro-3-methyl-quinolin-7-yl, 2-chloro-4-methyl-quinolin-7-yl, 2-methyl-8-fluoro-quinolin-2-yl, 2-methyl-quinolin-7-yl, 2-methyl-7-bromo-quinolin-7-yl, 3-methyl-7-bromo-quinolin-7-yl, 2-methyl-4-chloro-quinolin-7-yl, 4-methyl-7-bromo-quinolin-2-yl, 2-trifluoromethyl-quinolin-7-yl, 2-oxo-quinolin-7-yl, 2-carboxy-quinolin-7-yl, 2-aminocarbonyl-quinolin-7-yl, isoquinolin-3-yl, isoquinolin-5-yl, isoquinolin-6-yl, isoquinolin-7-yl, 1-chloro-isoquinolin-6-yl, 3-chloro-isoquinolin-6-yl, 3-fluoro-isoquinolin-6-yl, 6-bromo-isoquinolin-3-yl, 1-methoxy-isoquinolin-6-yl, 3-methoxy-isoquinolin-6-yl, 1-amino-isoquinolin-6-yl, 3-amino-isoquinolin-6-yl, 1-oxo-isoquinolin-6-yl, quinazolin-7-yl, quinoxalin-6-yl, indazol-3-yl, indazol-4-yl, indazol-5-yl, indazol-6-yl, 4-chloro-indazol-5-yl, 1-methyl-indazol-3-yl, 1-methyl-indazol-4-yl, 1-methyl-indazol-5-yl, 1-methyl-indazol-6-yl, 2-methyl-indazol-4-yl, 2-methyl-indazol-5-yl, 2-methyl-indazol-6-yl, 1,3-dimethyl-indazol-5-yl, 1,4-dimethyl-indazol-5-yl, 1,7-dimethyl-indazol-5-yl, 1,8-dimethyl-indazol-5-yl, 1-ethyl-indazol-5-yl, 2-ethyl-indazol-5-yl, 1-isopropyl-indazol-5-yl, 2-isopropyl-indazol-5-yl, 1-(2-hydroxyethyl)-indazol-5-yl, 2-(2-hydroxyethyl)-indazol-5-yl, 1-(2-hydroxyethyl)-6-fluoro-indazol-5-yl, 2-(2-hydroxyethyl)-6-fluoro-indazol-5-yl, 1-methyl-3-chloro-indazol-5-yl, 1-methyl-3-chloro-indazol-6-yl, 1-methyl-3-amino-indazol-6-yl, 1-methyl-3-aminocarbonyl-indazol-6-yl, 1-methyl-3-cyano-indazol-5-yl, 1-methyl-3-cyano-indazol-6-yl, 1-methyl-3-methoxy-indazol-5-yl, 1-methyl-3-methoxymethyl-indazol-5-yl, 1-methyl-3-methoxymethyl-indazol-6-yl, 1-methyl-7-methoxymethyl-indazol-4-yl, 1-methyl-3-hydroxymethyl-indazol-5-yl, 1-methyl-3-hydroxymethyl-indazol-6-yl, 1-methyl-7-hydroxymethyl-indazol-4-yl, 1-methyl-3-cyclopropyl-indazol-5-yl, 2-methyl-3-cyano-indazol-5-yl, 2-methyl-3-hydroxymethyl-indazol-5-yl, 2-methyl-3-methoxymethyl-indazol-5-yl, 1-(2-hydroxyethyl)-indazol-5-yl, 2-(2-hydroxyethyl)-indazol-5-yl), 1-(2-cyanoethyl)-indazol-5-yl, 2-(2-cyanoethyl)-indazol-5-yl, 1-oxetan-3-yl-indazol-5-yl, 1-cyclopropyl-indazol-5-yl, 1-cyclopropylmethyl-indazol-5-yl, 2-cyclopropylmethyl-indazol-5-yl, benzofuran-5-yl, benzofuran-6-yl, 2-methyl-benzofuran-5-yl, 2,3-dimethyl-benzofuran-5-yl, 2-cyano-benzofuran-5-yl, benzimidazol-2-yl, benzimidazol-5-yl, 1-methyl-benzimidazol-2-yl, 1,2-dimethyl-benzimidazol-6-yl, 1-methyl-6-fluoro-benzimidazol-2-yl, 2-oxo-benzimidazol-5-yl, benzoxazol-2-yl, benzoxazol-5-yl, 6-chloro-benzoxazol-2-yl, benzisoxazol-5-yl, benzthiazol-2-yl, benzthiazol-5-yl, 5-fluoro-benzothiazol-2-yl, 6-fluoro-benzothiazol-2-yl, 5-chloro-benzothiazol-2-yl, 6-chloro-benzothiazol-2-yl, 5,6-difluoro-benzothiazol-2-yl, 2-methyl-benzothiazol-5-yl, 2-methyl-benzothiazol-6-yl, 6-methyl-benzothiazol-2-yl, 2-methyl-benzothiazol-5-yl, 5-cyano-benzothiazol-2-yl, 6-cyano-benzothiazol-2-yl, benzothien-5-yl, 2-methyl-benzothien-5-yl, 2,3-dimethyl-benzothien-5-yl, 2,3-dihydro-benzofuran-5-yl, 2-oxo-3,4-dihydro-quinolin-7-yl, 1,2,3,4-tetrahydro-2-methylcarbonyl-isoquinolin-6-yl, 1,2,3,4,4a,8a-hexahydro-2-methyl-carbonyl-isoquinolin-6-yl, 2,3-dihydro-benzo[1,4]dioxin-6-yl, 2,3-dihydrobenzofuran-5-yl, 1,2-dimethyl-1,2-dihydro-3-oxo-indazol-5-yl, 2-oxo-3,4-dihydro-quinolin-6-yl, benzo[1,3]dioxol-5-yl, pyrrolo[2,3-b]pyridin-5-yl, 1-methyl-pyrazolo[4,3-b]pyridin-5-yl, [1,2,4]triazo[4,3-a]pyridin-6-yl, 3-methyl-[1,2,4]triazo[4,3-a]pyridin-6-yl, and 4-methyl-3,4-dihydro-pyhdo[3,2-b][1,4]oxazin-7-yl.

In another preferred embodiment,

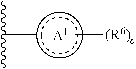

is selected from the group consisting of 4-cyano-phenyl, 3-hydroxy-phenyl, 4-hydroxy-phenyl, 3-fluoro-phenyl, 4-fluoro-phenyl, 3-chloro-phenyl, 4-chloro-phenyl, 2,4-dichloro-phenyl, 2-fluoro-4-chloro-phenyl, 3-chloro-4-fluoro-phenyl, 3-methyl-phenyl, 4-methyl-phenyl, 3-trifluoromethyl-phenyl, 3-methoxy-phenyl, 4-methoxy-phenyl, 3-aminocarbonyl-phenyl, 3-dimethylamino-phenyl, 4-dimethylamino-phenyl, 3-methylsulfonyl-amino-phenyl, 3-(cyclopropyl-sulfonylamino)-phenyl, 3-(cyclopropyl-carbonylamino)-phenyl, 3-(cyclopropyl-thio)-phenyl, 3-(cyclopropyl-sulfonyl)-phenyl, naphtha-2-yl, 6-fluoro-naphth-2-yl, 8-fluoro-naphth-2-yl, 6-chloro-naphth-2-yl, 7-fluoro-naphth-2-yl, 8-fluoro-naphth-2-yl, 6-methyl-naphth-2-yl, 5-methoxy-naphth-2-yl, 6-methoxy-naphth-2-yl, 8-methoxy-naphth-2-yl, 6-isopropoxy-naphth-2-yl, 6-cyano-naphth-2-yl, 7-methoxy-naphth-2-yl, 7-cyano-naphth-2-yl, 6-amino-pyridin-2-yl, isochroman-6-yl, isochroman-7-yl, 2-oxo-indolin-5-yl, indol-3-yl, indol-4-yl, indol-5-yl, indol-6-yl, 1-methyl-indol-5-yl, 1-methyl-indol-6-yl, 2-methyl-indol-5-yl, 1,2-dimethyl-indol-5-yl, 1,3-dimethyl-indol-5-yl, 2,3-dimethyl-indol-5-yl, 2-hydroxymethyl-indol-5-yl, 3-(2-hydroxyethyl-indol-5-yl), 3-cyanomethyl-indol-5-yl, 1-methyl-3-(2-hydroxyethyl)-indol-5-yl, quinolin-2-yl, quinolin-3-yl, quinolin-5-yl, quinolin-6-yl, quinolin-7-yl, 2-chloro-quinolin-7-yl, 4-chloro-quinolin-7-yl, 6-fluoro-quinolin-2-yl, 8-fluoro-quinolin-2-yl, 3-chloro-quinolin-7-yl, 2-methyl-quinolin-6-yl, 2-methyl-quinolin-6-yl, 4-methyl-quinolin-7-yl, 2-cyano-quinolin-6-yl, 2-chloro-3-methyl-quinolin-7-yl, isoquinolin-3-yl, isoquinolin-5-yl, isoquinolin-6-yl, isoquinolin-7-yl, 3-fluoro-isoquinolin-6-yl, 1-chloro-isoquinolin-6-yl, 3-chloro-isoquinolin-6-yl, 1-methoxy-isoquinolin-6-yl, 3-methoxy-isoquinolin-6-yl, 1-amino-isoquinolin-6-yl, 3-amino-isoquinolin-6-yl, 1-oxo-isoquinolin-6-yl, quinazolin-7-yl, indazol-3-yl, indazol-4-yl, indazol-5-yl, indazol-6-yl, 1-methyl-indazol-5-yl, 1-methyl-indazol-6-yl, 1-methyl-indazol-3-yl, 1-methyl-indazol-4-yl, 1-methyl-indazol-5-yl, 1-methyl-indazol-6-yl, 2-methyl-indazol-4-yl, 2-methyl-indazol-5-yl, 2-methyl-indazol-6-yl, 1,3-dimethyl-indazol-5-yl, 1,4-dimethyl-indazol-5-yl, 1,8-dimethyl-indazol-5-yl, 1-ethyl-indazol-5-yl, 1-methyl-3-chloro-indazol-5-yl, 1-methyl-3-chloro-indazol-6-yl, 1-methyl-3-aminocarbonyl-indazol-6-yl, 1-methyl-3-cyano-indazol-6-yl, 1-methyl-3-amino-indazol-6-yl, 1-methyl-3-methoxy-indazol-5-yl, 1-methyl-3-methoxymethyl-indazol-5-yl, 1-methyl-3-methoxymethyl-indazol-6-yl, 1-methyl-3-hydroxymethyl-indazol-5-yl, 1-methyl-3-hydroxymethyl-indazol-6-yl, 1-methyl-3-cyclopropyl-indazol-5-yl, 1-(cyclopropylmethyl)-indazol-5-yl, benzofuran-5-yl, benzofuran-6-yl, 2-methyl-benzofuran-5-yl, 2-cyano-benzofuran-5-yl, 2,3-dimethyl-benzofuran-5-yl, benzoxazol-2-yl, benzoxazol-5-yl, 6-chloro-benzoxazol-2-yl, benzimidazol-2-yl, benzimidazol-5-yl, 1-methyl-benzimidazol-5-yl, 2-oxo-benzimidazol-5-yl, benzothiazol-2-yl, benzthiazol-5-yl, 5-chloro-benzothiazol-2-yl, 5-fluoro-benzothiazol-2-yl, 6-fluoro-benzothiazol-2-yl, 6-chloro-benzothiazol-2-yl, 5,6-difluoro-benzothiazol-2-yl, 2-methyl-benzothiazol-5-yl, 2-methyl-benzothiazol-6-yl, 5-cyano-benzothiazol-2-yl, 6-cyano-benzthiazol-2-yl, benzothien-5-yl, 2-methyl-benzothien-5-yl, 2,3-dimethyl-benzothien-5-yl, 2,3-dihydrobenzofuran-5-yl, 2-oxo-3,4-dihydro-quinolin-6-yl, benzo[1,3]dioxol-5-yl, 1,8-naphthyridin-2-yl, and pyrrolo[2,3-b]pyridin-5-yl.

In another preferred embodiment,

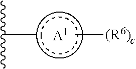

is selected from the group consisting of 4-cyano-phenyl, 3-hydroxy-phenyl, 4-fluoro-phenyl, 3-chloro-phenyl, 4-chloro-phenyl, 2-fluoro-4-chloro-phenyl, 3-chloro-4-fluoro-phenyl, 2-fluoro-4-cyano-phenyl, 2,4-dichloro-phenyl, 3-methyl-phenyl, 4-methyl-phenyl, 3-methoxy-phenyl, 4-methoxy-phenyl, 4-dimethylamino-phenyl, 3-(cyclopropyl-sulfonylamino)-phenyl, 3-(cyclopropyl-carbonylamino)-phenyl, 3-(cyclopropyl-thio)-phenyl, naphth-2-yl, 6-fluoro-naphth-2-yl, 7-fluoro-naphth-2-yl, 8-fluoro-naphth-2-yl, 6-chloro-naphth-2-yl, 6-methyl-naphth-2-yl, 6-methoxy-naphth-2-yl, 8-methoxy-naphth-2-yl, 6-cyano-naphth-2-yl, indol-3-yl, indol-4-yl, indol-5-yl, indol-6-yl, 1-methyl-indol-5-yl, 1-methyl-indol-6-yl, 2-methyl-indol-5-yl, 2,3-dimethyl-indol-5-yl, 2-(hydroxymethyl)-indol-5-yl, 3-(2-hydroxyethyl)-indol-5-yl, 3-(cyanomethyl)-indol-5-yl, 1-methyl-3-(2-hydroxyethyl)-indol-5-yl, 2-oxo-indolin-5-yl, quinolin-2-yl, quinolin-3-yl, quinolin-5-yl, quinolin-6-yl, quinolin-7-yl, 3-chloro-quinolin-7-yl, 6-fluoro-quinolin-2-yl, 8-fluoro-quinolin-2-yl, 8-fluoro-quinolin-7-yl, 4-methyl-quinolin-7-yl, 2-cyano-quinolin-6-yl, isoquinolin-5-yl, isoquinolin-6-yl, isoquinolin-7-yl, 6-fluoro-isoquinolin-6-yl, 1-amino-isoquinolin-6-yl, 3-amino-isoquinolin-6-yl, quinazolin-7-yl, indazol-3-yl, indazol-4-yl, indazol-5-yl, indazol-6-yl, 1-methyl-indazol-4-yl, 1-methyl-indazol-5-yl, 1-methyl-indazol-6-yl, 2-methyl-indazol-6-yl, 1,3-dimethyl-indazol-5-yl, 1,4-dimethyl-indazol-5-yl, 1-methyl-3-amino-indazol-6-yl, 1-methyl-3-aminocarbonyl-indazol-6-yl, 1-methyl-3-methoxymethyl-indazol-5-yl, 1-methyl-3-methoxymethyl-indazol-6-yl, 1-methyl-3-cyclopropyl-indazol-5-yl, benzofuran-5-yl, 2-methyl-benzofuran-5-yl, 2-cyano-benzofuran-5-yl, 2,3-dimethyl-benzofuran-5-yl, benzothiazol-2-yl, benzothiazol-5-yl, 6-fluoro-benzothiazol-2-yl, 6-chloro-benzothiazol-2-yl, 2-methyl-benzothiazol-5-yl, 6-methyl-benzothiazol-2-yl, 6-cyano-benzothiazol-2-yl, benzoxazol-2-yl, benzimidazol-5-yl, 1-methyl-benzimidazol-5-yl, benzothien-5-yl, 2-methyl-benzothien-5-yl, 2,3-dimethyl-benzothien-5-yl, and pyrrolo[2,3-b]pyridin-5-yl.

In another preferred embodiment,

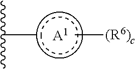

is selected from the group consisting of 3-cyano-phenyl, 4-cyano-phenyl, 3-hydroxy-phenyl, 4-hydroxy-phenyl, 3-fluoro-phenyl, 4-fluoro-phenyl, 3-chloro-phenyl, 4-chloro-phenyl, 2,4-dichloro-phenyl, 3-methyl-phenyl, 4-methyl-phenyl, 3-trifluoromethyl-phenyl, 4-trifluoromethyl-phenyl, 2-methoxy-phenyl, 3-methoxy-phenyl, 4-methoxy-phenyl, 3-thfluoromethoxy-phenyl, 4-thfluoromethoxy-phenyl, 3-dimethylamino-phenyl, 4-dimethylamino-phenyl, 3-methylsulfonyl-amino-phenyl, 3-amino-4-hydroxy-phenyl, 3-formamido-4-hydroxy-phenyl, pyridin-2-yl, pyridin-3-yl, pyridin-4-yl, 1-methyl-pyrazol-4-yl, 1-methyl-pyrazol-5-yl, indol-4-yl, indol-5-yl, indol-6-yl, quinolin-5-yl, quinolin-6-yl, isoquinolin-5-yl, isoquinolin-6-yl, isoquinolin-7-yl, indazol-4-yl, indazol-5-yl, 1-methyl-indazol-5-yl, 1-methyl-indazol-6-yl, benzofuran-5-yl, 2-methyl-benzofuran-5-yl, benzimidazol-5-yl, benzoxazol-2-yl, benzoxazol-5-yl, benzthiazol-5-yl, 2,3-dimethyl-benzothiophene-5-yl, 1,2,3,4-tetrahydro-2-methylcarbonyl-isoquinolin-6-yl, and 1,2,3,4,4a,8a-hexahydro-2-methyl-carbonyl-isoquinolin-6-yl.

In another preferred embodiment,

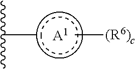

is selected from the group consisting of 3-hydroxy-phenyl, naphth-2-yl, 6-fluoro-naphth-2-yl, 7-fluoro-naphth-2-yl, 8-fluoro-naphth-2-yl, 6-chloro-naphth-2-yl, 6-methyl-naphth-2-yl, 6-methoxy-naphth-2-yl, 8-methoxy-naphth-2-yl, 6-cyano-naphth-2-yl, indol-3-yl, indol-5-yl, indol-6-yl, 1-methyl-indol-5-yl, 2-methyl-indol-5-yl, 2,3-dimethyl-indol-5-yl, 3-cyanomethyl-indol-5-yl, 2-hydroxymethyl-indol-5-yl, 3-(2-hydroxyethyl)-indol-5-yl, quinolin-3-yl, quinolin-5-yl, quinolin-7-yl, 3-chloro-quinolin-7-yl, 6-fluoro-quinolin-2-yl, 8-fluoro-quinolin-2-yl, 2-cyano-quinolin-6-yl, isoquinolin-6-yl, indazol-4-yl, indazol-5-yl, indazol-6-yl, 1-methyl-indazol-5-yl, 2-methyl-indazol-6-yl, benzofuran-5-yl, 2-methyl-benzofuran-5-yl, 2-cyano-benzofuran-5-yl, benzothiazol-2-yl, benzthiazol-5-yl, 6-chloro-benzothiazol-2-yl, 6-methyl-benzothiazol-2-yl, 6-cyano-benzothiazol-2-yl, benzoxazol-2-yl, benzimidazol-5-yl, 1-methyl-benzimidazol-5-yl, benzothien-5-yl, 2-methyl-benzothien-5-yl, and 2,3-dimethyl-benzothien-5-yl.

In another preferred embodiment,

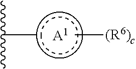

is selected from the group consisting of 4-cyano-phenyl, 3-hydroxy-phenyl, 4-hydroxy-phenyl, 3-fluoro-phenyl, 4-fluoro-phenyl, 3-chloro-phenyl, 4-chloro-phenyl, 2,4-dichloro-phenyl, 3-methyl-phenyl, 4-methyl-phenyl, 3-trifluoromethyl-phenyl, 3-methoxy-phenyl, 4-methoxy-phenyl, 3-dimethylamino-phenyl, 4-dimethylamino-phenyl, 3-methylsulfonyl-amino-phenyl, indol-4-yl, indol-5-yl, indol-6-yl, quinolin-5-yl, quinolin-6-yl, isoquinolin-5-yl, isoquinolin-6-yl, isoquinolin-7-yl, indazol-4-yl, indazol-5-yl, 1-methyl-indazol-5-yl, 1-methyl-indazol-6-yl, benzofuran-5-yl, 2-methyl-benzofuran-5-yl, benzoxazol-2-yl, benzoxazol-5-yl, benzthiazol-5-yl, and 2,3-dimethyl-benzothiophen-5-yl.

In another preferred embodiment,

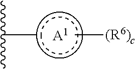

is selected from the group consisting of naphtha-2-yl, 6-chloro-naphth-2-yl, 6-fluoro-naphth-2-yl, 6-methyl-naphth-2-yl, 6-methoxy-naphth-2-yl, 6-cyano-naphth-2-yl, indol-5-yl, indol-6-yl, 1-methyl-indol-5-yl, 2-methyl-indol-5-yl, 2-hydroxymethyl-indol-5-yl, 3-(2-hydroxyethyl)-indol-5-yl, 3-cyanomethyl-indol-5-yl, indazol-5-yl, indazol-6-yl, 1-methyl-indazol-5-yl, quinolin-7-yl, 3-chloro-quinolin-7-yl, 6-fluoro-quinolin-2-yl, 8-fluoro-quinolin-2-yl, isoquinolin-6-yl, benzofuran-5-yl, 2-methyl-benzofuran-5-yl, 2-cyano-benzofuran-5-yl, benzothien-5-yl, 2-methyl-benzothien-5-yl, 2,3-dimethyl-benzothien-5-yl, benzoxazol-2-yl, benzothiazol-2 yl, and 1-methyl-benzimidazol-5-yl.

In another preferred embodiment,

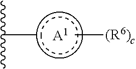

is selected from the group consisting of naphth-2-yl, 6-chloro-naphth-2-yl, 6-fluoro-naphth-2-yl, 7-fluoro-naphth-2-yl, 8-fluoro-naphth-2-yl, 6-methyl-naphth-2-yl, 6-methoxy-naphth-2-yl, 6-cyano-naphth-2-yl, indol-3-yl, indol-5-yl, indol-6-yl, 1-methyl-indol-5-yl, 2-methyl-indol-6-yl, 3-(2-hydroxyethyl)-indol-5-yl, 3-cyanomethyl-indol-5-yl, 1,3-dimethyl-indol-5-yl, 1-methyl-3-(2-hydroxyethyl)-indol-5-yl, quinolin-7-yl, 3-chloro-quinolin-7-yl, 6-fluoro-quinolin-6-yl, isoquinolin-6-yl, quinazolin-7-yl, indazol-4-yl, indazol-5-yl, indazol-6-yl, 1-methyl-indazol-5-yl, 2-methyl-indazol-6-yl, 1-methyl-3-amino-indazol-6-yl, 1-methyl-3-aminocarbonyl-indazol-6-yl, benzofuran-5-yl, 2-methyl-benzofuran-5-yl, 2-methyl-benzothien-5-yl, benzothiazol-5-yl, 6-chloro-benzothiazol-2-yl, 6-methyl-benzothiazol-2-yl, 6-cyano-benzothiazol-2-yl, benzimidazol-5-yl, and 1-methyl-benzimidazol-5-yl.

In another preferred embodiment,

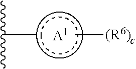

is selected from the group consisting of 4-cyano-phenyl, 3-hydroxy-phenyl, 4-fluoro-phenyl, 3-chloro-phenyl, 4-chloro-phenyl, 2,4-dichloro-phenyl, 3-methyl-phenyl, 4-methyl-phenyl, 3-methoxy-phenyl, 4-methoxy-phenyl, 4-dimethylamino-phenyl, indol-4-yl, indol-5-yl, indol-6-yl, isoquinolin-5-yl, isoquinolin-6-yl, isoquinolin-7-yl, indazol-4-yl, indazol-5-yl, 1-methyl-indazol-5-yl, 1-methyl-indazol-6-yl, benzofuran-5-yl, 2-methyl-benzofuran-5-yl, benzthiazol-5-yl, and 2,3-dimethyl-benzothiophen-5-yl.

In another preferred embodiment,

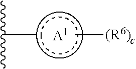

is selected from the group consisting of indol-5-yl, indol-6-yl, indazol-4-yl, indazol-5-yl, 1-methyl-indazol-5-yl, benzthiazol-5-yl, benzofuran-5-yl, benzothien-5-yl, and 6-cyano-naphth-2-yl.

In another preferred embodiment,

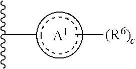

is selected from the group consisting of 3-hydroxy-phenyl, indol-5-yl, indol-6-yl, isoquinolin-6-yl, indazol-4-yl, 1-methyl-indazol-5-yl, benzofuran-5-yl, and benzthiazol-5-yl.

In another preferred embodiment,

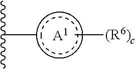

is selected from the group consisting of 4-cyano-phenyl, 3-hydroxy-phenyl, 3-chloro-phenyl, 4-chloro-phenyl, 4-methyl-phenyl, 4-methoxy-phenyl, indol-4-yl, indol-5-yl, indol-6-yl, quinolin-5-yl, isoquinolin-6-yl, isoquinolin-7-yl, indazol-4-yl, indazol-5-yl, 1-methyl-indazol-5-yl, 1-methyl-indazol-6-yl, benzofuran-5-yl, and benzthiazol-5-yl.

In another preferred embodiment,

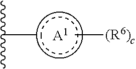

is selected from the group consisting of indol-5-yl, indol-6-yl, isoquinolin-6-yl, and benzofuran-5-yl.

In a preferred embodiment, the present invention is directed to compounds of formula (XXVIII) wherein c is an integer from 0 to 2.

In a preferred embodiment, the present invention is directed to compounds of formula (XXVIII) wherein each R⁶ is independently selected from the group consisting of hydroxy, oxo, halogen, cyano, nitro, C₁₋₄alkyl, fluorinated C₁₋₄alkyl, hydroxy substituted C₁₋₄alkyl, cyano substituted (C₁₋₄alkyl), —(C₁₋₂ alkyl)-O—(C₁₋₄alkyl), C₁₋₄alkoxy, fluorinated C₁₋₄alkoxy, —SO₂—(C₁₋₄alkyl), —C(O)—(C₁₋₄alkyl), —C(O)-(fluorinated C₁₋₂alkyl), —C(O)OH, —C(O)O—(C₁₋₄alkyl), —C(O)—NR^{M}R^{N}, —NR^{M}R^{N}, —NR^{M}—C(O)H, —NR^{M}—SO₂—(C₁₋₄alkyl), C₃₋₅ cycloalkyl, 1-cyano-(C₃₋₅cycloalkyl), —(C₁-2alkyl)-(C₃₋₅cycloalkyl), —S—(C₃₋₅cycloalkyl), —SO₂—(C₃₋₅cycloalkyl), —NH—(C₃₋₅cycloalkyl), —NH—SO₂—(C₃₋₅cycloalkyl), oxetanyl, and tetrahydro-furanyl; wherein R^{M} and R^{N} are each independently selected from the group consisting of hydrogen and C₁₋₄alkyl; wherein

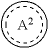

is selected from the group consisting of phenyl and 5 to 6 membered heteroaryl.

In another preferred embodiment, the present invention is directed to compounds of formula (XXVIII) wherein each R⁶ is independently selected from the group consisting of hydroxy, oxo, halogen, cyano, C₁₋₄alkyl, fluorinated C₁₋₂alkyl, hydroxy substituted C₁₋₄alkyl, cyano-substituted C₁₋₂alkyl, —(C₁₋₂alkyl)-O—(C₁₋₂alkyl), C₁₋₄alkoxy, fluorinated C₁₋₂alkoxy, —SO₂—(C₁₋₄alkyl), —CO²H, —C(O)O—(C₁₋₂alkyl), —C(O)—(C₁₋₂alkyl), —C(O)-(fluorinated C₁₋₂alkyl), —C(O)—NR^{M}R^{N}, —NR^{M}R^{N}, —NR^{M}—C(O)H, —NR^{M}—SO₂—(C-2alkyl), C₃₋₅cycloalkyl, 1-cyano-cyclopropyl, —(C₁₋₂alkyl)-(C₃₋₅cycloalkyl), —S—(C₃₋₅cycloalkyl), —SO₂—(C₃₋₅cycloalkyl), —NH—C(O)—(C₃₋₅cycloalkyl) and —NH—SO₂—(C₃₋₅cycloalkyl), and oxetan-3-yl; and wherein R^{M} and R^{N} are each independently selected from the group consisting of hydrogen and C₁₋₂alkyl.

In another preferred embodiment, the present invention is directed to compounds of formula (XXVIII) wherein each R⁶ is independently selected from the group consisting of hydroxy, halogen, cyano, nitro, C₁₋₄alkyl, fluorinated C₁₋₄alkyl, hydroxy substituted C₁₋₄alkyl, C₁₋₄alkoxy, fluorinated C₁₋₄alkoxy, —NR^{M}R^{N}, —C(O)—(C₁₋₄alkyl), —C(O)—NR^{M}R^{N}, —C(O)OH, —C(O)O—(C₁₋₄alkyl), —NR^{M}—C(O)H, and —NR^{M}—SO₂—(C₁₋₄ alkyl); wherein R^{M} and R^{N} are each independently selected from the group consisting of hydrogen and C₁₋₄alkyl. In another preferred embodiment, the present invention is directed to compounds of formula (XXVIII) wherein each R⁶ is independently selected from the group consisting of hydroxy, halogen, cyano, C₁₋₂alkyl, fluorinated C₁₋₂alkyl, C₁₋₂alkoxy, fluorinated C₁₋₂alkoxy, —NR^{M}R^{N}, —C(O)—(C₁₋₂alkyl), —NR^{M}—C(O)H and —NR^{M}—SO₂—(C₁₋₂alkyl); and wherein R^{M} and R^{N} are each independently selected from the group consisting of hydrogen and C₁₋₂alkyl.

In another preferred embodiment,

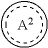

is selected from the group consisting of phenyl and 5 to 6 membered heteroaryl. In another preferred embodiment, the present invention is directed to compounds of formula (XXVIII) wherein

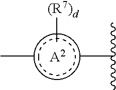

is selected from the group consisting of phenyl and 6 membered, nitrogen containing heteroaryl. In another preferred embodiment,

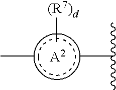

is selected from the group consisting of phenyl, pyridin-3-yl, pyridin-4-yl, and pyrazol-4-yl. In another preferred embodiment, the present invention is directed to compounds of formula (XXVIII) wherein

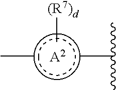

is selected from the group consisting of phenyl, pyridin-3-yl and pyridin-4-yl. In another preferred embodiment, the present invention is directed to compounds of formula (XXVIII) wherein

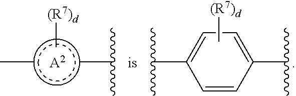

In another preferred embodiment,

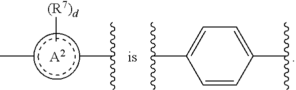

In a preferred embodiment, the present invention is directed to compounds of formula (XXVIII) wherein d is an integer from 0 to 1.

In a preferred embodiment, the present invention is directed to compounds of formula (XXVIII) wherein R⁷ is selected from the group consisting of hydroxy, halogen, cyano, C₁₋₄alkyl, fluorinated C₁₋₄ alkyl, hydroxy substituted C₁₋₄alkyl, C₁₋₄alkoxy and fluorinated C₁₋₄alkoxy.

In a preferred embodiment, the present invention is directed to compounds of formula (XXVIII) wherein

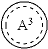

is selected from the group consisting of phenyl, 5 to 6 membered saturated heterocyclyl and 5 to 6 membered heteroaryl. In another preferred embodiment, the present invention is directed to compounds of formula (XXVIII) wherein

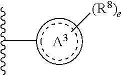

is selected from the group consisting of phenyl, 5 to 6 membered, saturated, nitrogen containing heterocyclyl and 5 to 6 membered, nitrogen containing heteroaryl. In another preferred embodiment, the present invention is directed to compounds of formula (XXVIII) wherein

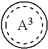

is selected from the group consisting of phenyl and 5 to 6 membered heteroaryl. In another preferred embodiment, the present invention is directed to compounds of formula (XXVIII) wherein

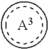

is selected from the group consisting of phenyl and 5 to 6 membered nitrogen containing heteroaryl.

In another preferred embodiment,

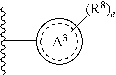

is selected from the group consisting of 4-bromo-phenyl, 3-chloro-phenyl, 4-methyl-phenyl, pyridin-3-yl, pyridin-4-yl, 1-methyl-pyrazol-3-yl, 1-methyl-pyrazol-4-yl, 1-methyl-pyrazol 5-yl, 1-isopropyl-pyrazol-4-yl, 1-isobutyl-pyrazol-5-yl, 1-(2-methylpropyl)-pyrazol-3-yl, 1-cyclopropyl-pyrazol-4-yl, 1-cyclobutyl-pyrazol-4-yl, 1-cyclopropylmethyl-pyrazol-3-yl, 1-cyclopropylmethyl-pyrazol-5-yl, 1,2,3,4-tetrazol-5-yl, pyrazol-3-yl, pyrrolidin-1-yl, morpholin-4-yl, 4-methyl-piperazin-1-yl, imidazol-1-yl, piperazin-1-yl, 4-methyl-piperazin-1-yl, and 1-(oxetan-3-yl)-pyrazol-4-yl.

In another preferred embodiment,

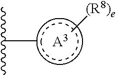

is selected from the group consisting of 4-bromo-phenyl, 3-chloro-phenyl, 4-methyl-phenyl, pyridin-3-yl, pyridin-4-yl, 1-methyl-pyrazol-3-yl, 1-(cyclopropylmethyl)-pyrazol-3-yl, 1-(2-methylpropyl)-pyrazol-3-yl, 1-methyl-pyrazol-4-yl, 1-isopropyl-pyrazol-4-yl, 1-cyclopropyl-pyrazol-4-yl, 1-cyclobutyl-pyrazol-4-yl, 1-methyl-pyrazol-5-yl, 1-isobutyl-pyrazol-5-yl, 1-(cyclopropylmethyl)-pyrazol-5-yl, tetrazol-5-yl, 5-methyl-oxadiazol-2-yl, piperazin-1-yl, 4-methyl-piperazin-1-yl, pyrrolidin-1-yl, morpholin-4-yl, imidazol-1-yl, and oxetan-3-yl.

In another preferred embodiment,

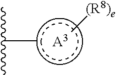

is selected from the group consisting of pyridin-3-yl, pyridin-4-yl, 1-methyl-pyrazol-4-yl, 1-isopropyl-pyrazol-4-yl, 1-cyclopropyl-pyrazol-4-yl, 1-cyclobutyl-pyrazol-4-yl, 1-methyl-pyrazol-5-yl, and 5-methyl-oxadiazol-2-yl.

In another preferred embodiment,

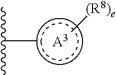

is selected from the group consisting of 4-bromo-phenyl, pyridin-3-yl, pyridin-4-yl, 1-methyl-pyrazol-4-yl, 1-methyl-pyrazol-5-yl, tetrazol-5-yl, and pyrazol-3-yl.

In another preferred embodiment,

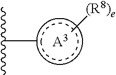

is selected from the group consisting of 4-bromo-phenyl, pyridin-3-yl, pyridin-4-yl, 1-methyl-pyrazol-4-yl, 1-methyl-pyrazol-5-yl, tetrazol-5-yl, and pyrazol-3-yl.

In another preferred embodiment,

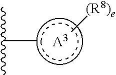

is selected from the group consisting of 1-methyl-pyrazol 4-yl, 1-isopropyl-pyrazol-4-yl, 1-cyclopropyl-pyrazol-4-yl, 1-cyclobutyl-pyrazol-4-yl, 1-methyl-pyrazol-5-yl, and pyridin-4-yl.

In another preferred embodiment,

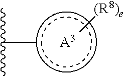

is selected from the group consisting of pyridin-3-yl and 1-methyl-pyrazol-4-yl.

In a preferred embodiment, the present invention is directed to compounds of formula (XXVIII) wherein e is an integer from 0 to 2. In another preferred embodiment, the present invention is directed to compounds of formula (XXVIII) wherein e is an integer from 0 to 1.

In a preferred embodiment, the present invention is directed to compounds of formula (XXVIII) wherein each R⁸ is independently selected from the group consisting of hydroxy, halogen, cyano, C₁₋₄ alkyl, fluorinated C₁-alkyl, hydroxy substituted C₁₋₄alkyl, C₁₋₄alkoxy, fluorinated C₁₋₄alkoxy, —NR^{T}R^{U}, —C(O)—NR^{T}R^{U}, —C(O)OH, —C(O)O—(C₁₋₄alkyl), —(C₁₋₄alkyl)-NR^{T}R^{U}, C₃₋₅cycloalkyl, —(C₁₋₂alkyl)-(C₃₋₅cycloalkyl), oxetanyl, and tetrahydro-furanyl; wherein R^{T} and R^{u} are each independently selected from the group consisting of hydrogen and d-4alkyl. In another preferred embodiment, the present invention is directed to compounds of formula (XXVIII) wherein R⁸ is selected from the group consisting of halogen, C₁₋₄ alkyl, C₃₋₅ cycloalkyl, —(C₁-2alkyl)-(C₃₋₅cycloalkyl), and oxetanyl.

In a preferred embodiment, the present invention is directed to compounds of formula (XXVIII) wherein each R⁸ is independently selected from the group consisting of hydroxy, halogen, cyano, C₁₋₄ alkyl, fluorinated C₁₋₄alkyl, hydroxy substituted C₁₋₄alkyl, C₁₋₄alkoxy, fluorinated C₁₋₄alkoxy, —NR^{T}R^{U}, —C(O)—NR^{T}R^{U}, —C(O)OH, —C(O)O—(C₁₋₄alkyl) and —(C₁₋₄alkyl)-NR^{T}R^{U}; wherein R^{T} and R^{u} are each independently selected from the group consisting of hydrogen and C₁₋₄alkyl. In another preferred embodiment, the present invention is directed to compounds of formula (XXVIII) wherein R⁸ is selected from the group consisting of halogen and

In a preferred embodiment, the present invention is directed to compounds of formula (XXVIII) selected from the group consisting of 5-[4-(1-Benzofuran-5-yl)phenyl]-6-{[1-(cyclopropylcarbonyl)azetidin-3-yl]methyl}-4,6-diazaspiro[2,4]hept-4-en-7-one; 6-{[1-(cyclopropylcarbonyl)azetidin-3-yl]methyl}-5-[4′-(1-methyl-1H-pyrazol-4-yl)biphenyl-4-yl]-4,6-diazaspiro[2,4]hept-4-en-7-one; (R)-6-((1-(cyclopropanecarbonyl)pyrrolidin-3-yl)methyl)-5-(4′-(1-methyl-1H-pyrazol-4-yl)-[1,1′ biphenyl]-4-yl)-4,6-diazaspiro[2,4]hept-4-en-7-one; (R)-6-((1-(cyclopropanecarbonyl)pyrrolidin-3-yl)methyl)-5-(4-(2-methyl-1H-indol-5-yl)phenyl)-4,6-diazaspiro[2,4]hept-4-en-7-one; 6-(4-(6-((1-(cyclopropanecarbonyl)azetidin-3-yl)methyl)-7-oxo-4,6-diazaspiro[2,4]hept-4-en-5-yl)-3-fluorophenyl)-2-naphthonitrile; (R)-6-((1-(cyclopropanecarbonyl)pyrrolidin-3-yl)methyl)-5-(2-methyl-4-(1-methyl-1H-indazol-5-yl)phenyl)-4,6-diazaspiro[2,4]hept-4-en-7-one; 6-((1-(cyclopropanecarbonyl)azetidin-3-yl)methyl)-5-(2-methyl-4-(1-methyl-1H-indazol-5-yl)phenyl)-4,6-diazaspiro[2,4]hept-4-en-7-one; 6-((1-(cyclopropanecarbonyl)azetidin-3-yl)methyl)-5-(2-fluoro-4-(1-methyl-1H-indazol-5-yl)phenyl)-4,6-diazaspiro[2,4]hept-4-en-7-one; 5-(4-(benzo[d]thiazol-2-yl)-2-fluorophenyl)-6-((1-(cyclopropanecarbonyl)azetidin-3-yl)methyl)-4,6-diazaspiro[2,4]hept-4-en-7-one; 6-((1-(cyclopropanecarbonyl)azetidin-3-yl)methyl)-5-(2-fluoro-4-(2-methyl-1H-indol-5-yl)phenyl)-4,6-diazaspiro[2,4]hept-4-en-7-one; 6-((1-(cyclopropanecarbonyl)azetidin-3-yl)methyl)-5-(2-fluoro-4-(1-methyl-1H-indol-5-yl)phenyl)-4,6-diazaspiro[2,4]hept-4-en-7-one; (R)-6-((1-(cyclopropanecarbonyl)pyrrolidin-3-yl)methyl)-5-(2-fluoro-4-(1-methyl-1H-indazol-5-yl)phenyl)-4,6-diazaspiro[2,4]hept-4-en-7-one, and stereoisomers, tautomers, and pharmaceutically acceptable salts thereof. In another preferred embodiment, the present invention is directed to compounds of formula (XXVIII) selected from the group consisting of 6-{[1-(cyclopropylcarbonyl)azetidin-3-yl]methyl}-5-[4′-(1-methyl-1H-pyrazol-4-yl)biphenyl-4-yl]-4,6-diazaspiro[2,4]hept-4-en-7-one; (R)-6-((1-(cyclopropanecarbonyl)pyrrolidin-3-yl)methyl)-5-(4′-(1-methyl-1H-pyrazol-4-yl)-[1,1′-biphenyl]-4-yl)-4,6-diazaspiro[2,4]hept-4-en-7-one; (R)-6-((1-(cyclopropanecarbonyl)pyrrolidin-3-yl)methyl)-5-(2-fluoro-4-(1-methyl-1H-indazol-5-yl)phenyl)-4,6-diazaspiro[2,4]hept-4-en-7-one; and stereoisomers, tautomers and pharmaceutically acceptable salts thereof. In an embodiment, the present invention is directed to compounds of formula (XXVIII) wherein R¹ and R² are taken together with the carbon atom to which they are bound to form a ring structure other than tetrahydrofuran-3,3-diyl or tetrahydropyran-4,4-diyl.

In an embodiment, the present invention is directed to compounds of formula (XXVIII) wherein (L¹)ₐ is other than-SCVpyrrolidin-1-yl or —SO₂-pyridin-3-yl. In another embodiment, the present invention is directed to compounds of formula (XXVIII) wherein (L¹)ₐ is other than —C(O)-thiazol-2-yl, —C(O)—CF₃, and —C(O)OCH₃

In an embodiment, the present invention is directed to compounds of formula (XXVIII) wherein R⁵ is other than 1,2,3,4,4a,8a-hexahydro-2-methyl-carbonyl-isoquinolin-6-yl), 1,2,3,4-trihydro-2-methylcarbonyl-isoquinolin-2-yl, 4-methyl-3,4-dihydro-pyrido[2,3-b]oxazon-7-yl, 2-oxo-3,4-dihydro-quinolin-7-yl, 5-chloro-pyridin-3-yl, 5-(2-hydroxy-2-methyl-propyl)-pyridin-2-yl, 6-isopropyl-pyridin-3-yl, 6-(1-cyanomethyl)-pyridin-3-yl, 6-(2-hydroxy-2-methyl-propyl)-pyridin-3-yl, 2-(piperazin-1-yl)-pyridin-4-yl, 2-(4-methyl-piperazin-1-yl)-pyridin-4-yl, 6-(morpholin-4-yl)-pyridin-3-yl, 1,5-naphthyridin-3-yl, 3-methyl-[1,2,4]triazolo[4,3-a]pyridin-6-yl, or 6-(4-methyl-piperazin-1-yl)-pyridin-3-yl.

In an embodiment, R¹ and R² are taken together with the carbon atom to which they are bound to form 1-(methoxycarbonyl)-azetidin-3-yl, m is 1 and n is 0 or m is 0 and n is 1;

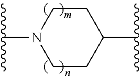

is pyrrolidin-3R-yl; -(L¹)ₐ-R³ is selected from the group consisting of —C(O)—CF₃, —C(O)-cyclopropyl, —C(O)-(thiazol-2-yl), —C(O)OCH₃, and —SO₂—CH₃;

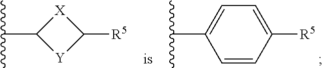

and b is 0; then R⁵ is other than quinolin-7-yl, benzofuran-5-yl, 1-methyl-indazol-5-yl, 1-methyl-pyrazol-4-yl, 4-(1-methyl-pyrazol-4-yl)-phenyl, 1,2,3,4,4a,8a-hexahydro-2-methyl-carbonyl-isoquinolin-6-yl), or 1,2,3,4-trihydro-2-methylcarbonyl-isoquinolin-2-yl.

In an embodiment, R¹ and R² are taken together with the carbon atom to which they are bound to form cyclopentyl m is 1 and n is 0 or m is 0 and n is 1;

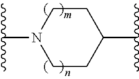

is pyrrolidin-3R-yl; -(L¹)ₐ-R³ is —SO₂-pyrrolidin-1-yl;

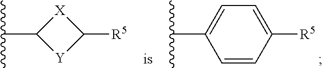

and b is 0 or (R⁴)_{b} is 2-methyl; then R⁵ is other than benzofuran-5-yl.

In another embodiment, when R¹ and R² are taken together with the carbon atom to which they are bound to form cyclopropyl; m is 0, n is 0,

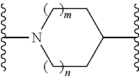

is azetidin-3-yl; -(L¹)ₐ-R³ is selected from the group consisting of —C(O)-cyclopropyl, —C(O)-(1-methyl-cyclopropyl) and —C(O)-(1-hydroxy-cyclopropyl);

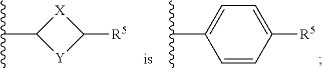

b is 0 or (R⁴)_{b} is selected from the group consisting of 2-fluoro and 2-methyl; then R⁵ is other than 1-isopropylsulfonyl-phenyl, 1-methyl-indazol-5-yl, 1-isopropyl-indazol-5-yl, 1-oxetan-3-yl, indazol-5-yl, 1-methyl-pyrazol-4-yl, 4-methyl-7-bromo-quinolin-2-yl, 5-(2-hydroxy-2-methyl-propyl)-pyridin-2-yl, 6-isopropyl-pyridin-3-yl, 6-(1-cyanomethyl)-pyridin-3-yl, 6-(2-hydroxy-2-methyl-propyl)-pyridin-3-yl, 1,5-naphthyridin-3-yl, 3-methyl-[1,2,4]triazolo[4,3-a]pyridin-6-yl, 4-(1-isobutyl-pyrazol-5-yl)-phenyl or 6-(morpholin-4-yl)-pyridin-3-yl.

In another embodiment, when R₁ and R² are taken together with the carbon atom to which they are bound to form cyclopropyl; m is 0, n is 0,

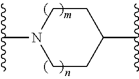

is azetidin-3-yl; -(L¹)ₐ-R³ is —C(O)-(1-hydroxy-cyclopropyl);

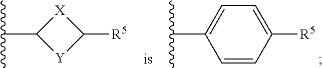

and (R⁴)_{b} is 2-methyl; then R⁵ is other than 1-methyl-indazol-5-yl.

In another embodiment, when R¹ and R² are taken together with the carbon atom to which they are bound to form cyclopropyl; m is 0, n is 0,

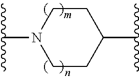

is azetidin-3-yl; -(L¹)ₐ-R³ is —C(O)-pyridin-3-yl;

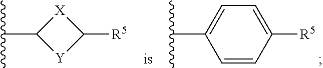

(R⁴)_{b} is 2-methyl, then R⁵ is other than 1-methyl-indazol-5-yl.

In another embodiment, when R¹ and R² are taken together with the carbon atom to which they are bound to form cyclopropyl; m is 0, n is 2,

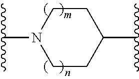

is piperidin-3R-yl or piperidin-3S-yl; -(L¹)ₐ-R³ is —C(O)-cyclopropyl;

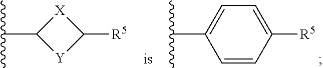

and b is 0, then R⁵ is other than indazol-5-yl, benzofuran-5-yl, benzothien-5-yl, 1-methyl-indazol-5-yl, 4-(4-methylphenyl)phenyl or 4-(3-chlorophenyl)-phenyl.

In another embodiment, when R¹ and R² are taken together with the carbon atom to which they are bound to form cyclopropyl, m is 1, n is 1,

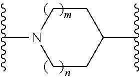

is piperidin-4-yl; -(L¹)ₐ-R³ is —C(O)-cyclopropyl;

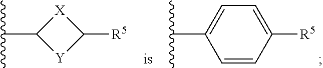

and b is 0, then R⁵ is other than 4-trifluoromethyl-phenyl, 1-methyl-pyrazol-4-yl, benzoxazol-5-yl, pyridine-4-yl or 4-(1-methyl-pyrazol-4-yl)-phenyl.

In another embodiment, when R¹ and R² are taken together with the carbon atom to which they are bound to form cyclopropyl; m is 0 and n is 1 or m is 1 and n is 0;

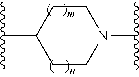

is pyrrolidin-3R-yl; -(L¹)ₐ-R³ is —C(O)-cyclopropyl;

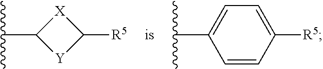

and b is 0, then R⁵ is other than 5-chloro-pyridin-3-yl, 2-oxo-3,4-dihydro-quinolin-7-yl or 6-(4-methyl-piperazin-1-yl)-pyridin-3-yl.

In another embodiment, when R¹ and R² are taken together with the carbon atom to which they are bound to form tetrahydrofuran-3,3-diyl or tetrahydropyran-4,4-diyl, m is an integer from 0 to 1 and n is 0 or m is 0 and n is an integer from 0 to 1;

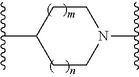

is selected from the group consisting of azetidin-3-yl, pyrrolidin-3R-yl, and pyrrolidin-3-yl; -(L¹)ₐ-R³ is selected from the group consisting of —C(O)-thiazol-2-yl, —C(O)—CF₃, —C(O)OCH₃, and —SO₂—CH₃,

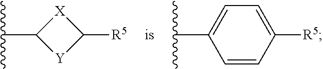

and b is 0, then R⁵ is other than quinolin-7-yl, 1-methyl-indazol-5-yl, benzofuran-5-yl, or 4-(1-methyl-pyrazol-4-yl)-phenyl.

In another embodiment, when R¹ and R² are taken together with the carbon atom to which they are bound to form 1-(methoxycarbonyl)-azetidin-3-yl, m is 1 and n is 0 or m is 0 and n is 1;

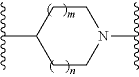

is pyrrolidin-3R-yl; -(L¹)ₐ-R³ is selected from the group consisting of —C(O)-cyclopropyl,

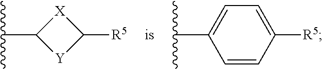

and b is 0; then R⁵ is other than quinolin-7-yl, benzofuran-5-yl, or 1-methyl-indazol-5-yl.

In some embodiments, when R¹ and R² are taken together with the carbon atom to which they are bound to form cyclopropyl; m is 0, n is 0,

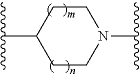

is azetidin-3-yl; -(L¹)ₐ-R³ is selected from the group consisting of —C(O)-cyclopropyl, —C(O)-(1-methyl-cyclopropyl) and —C(O)-(1-hydroxy-cyclopropyl);

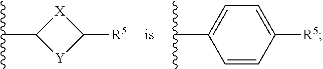

b is 0 or (R⁴)_{b} is selected from the group consisting of 2-fluoro and 2-methyl; then R⁵ is other than 1-methyl-indazol-5-yl or indazol-5-yl.

In some embodiments, when R¹ and R² are taken together with the carbon atom to which they are bound to form cyclopropyl; m is 1, n is 1,

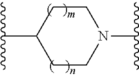

is piperidin-4-yl; -(L¹)ₐ-R³ is selected from the group consisting of —C(O)-cyclopropyl;

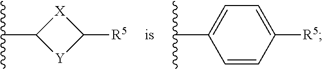

b is 0; then R⁵ is other than benzoxazol-5-yl.

In another embodiment, when R¹ and R² are taken together with the carbon atom to which they are bound to form cyclopropyl; m is 0 and n is 1 or m is 1 and n is 0;

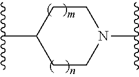

is pyrrolidin-3R-yl; -(L¹)ₐ-R³ is —C(O)-cyclopropyl;

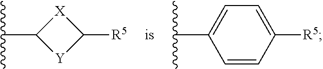

and b is 0, then R⁵ is other than 2-oxo-3,4-dihydro-quinolin-7-yl.

In another embodiment, when R¹ and R² are taken together with the carbon atom to which they are bound to form 1-(methoxycarbonyl)-azetidin-3-yl, m is 1 and n is 0 or m is 0 and n is 1;

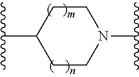

is pyrrolidin-3R-yl; -(L¹)ₐ-R³ is selected from the group consisting of —C(O)—CF₃, —C(O)-cyclopropyl, —C(O)-(thiazol-2-yl), —C(O)OCH₃, or —SO₂—CH₃,

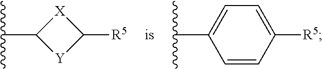

and b is 0; then R⁵ is other 4-(1-methyl-pyrazol-4-yl)-phenyl.

In another embodiment, when R¹ and R² are taken together with the carbon atom to which they are bound to form cyclopentyl; m is 1 and n is 0 or m is 0 and n is 1;

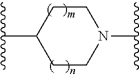

is pyrrolidin-3R-yl; -(L¹)ₐ-R³ is —C(O)-cyclopropyl;

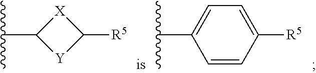

b is 0 or (R⁴)_{b} is 2-methyl; then R⁵ is other than 2-(piperazin-1-yl)-pyridin-4-yl or 2-(4-methyl-piperazin-1-yl)-pyridin-4-yl.

In another embodiment, when R¹ and R² are taken together with the carbon atom to which they are bound to form cyclopropyl; m is 0, n is 0,

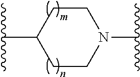

is azetidin-3-yl; -(L¹)ₐ-R³ is selected from the group consisting of —C(O)-cyclopropyl, —C(O)-(1-methyl-cyclopropyl) and —C(O)-(1-hydroxy-cyclopropyl);

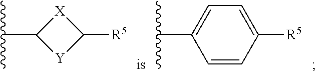

b is 0 or (R⁴)_{b} is selected from the group consisting of 2-fluoro and 2-methyl; then R⁵ is other than 4-(1-isobutyl-pyrazol-5-yl)-phenyl.

In another embodiment, when R¹ and R² are taken together with the carbon atom to which they are bound to form cyclopropyl; m is 0, n is 2,

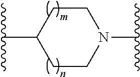

is piperidin-3R-yl; -(L¹)ₐ-R³ is —C(O)-cyclopropyl;

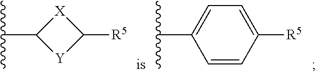

and b is 0, then R⁵ is other than 4-(4-methylphenyl)phenyl or 4-(3-chlorophenyl)-phenyl.

In another embodiment, when R¹ and R² are taken together with the carbon atom to which they are bound to form cyclopropyl, m is 1, n is 1,

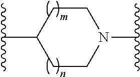

is piperidin-4-yl; -(L¹)ₐ-R³ is —C(O)-cyclopropyl;

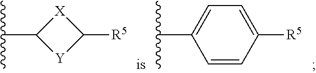

and b is 0, then R⁵ is other than 4-(1-methyl-pyrazol-4-yl)-phenyl.

In another embodiment, when R¹ and R² are taken together with the carbon atom to which they are bound to form cyclopropyl; m is 0 and n is 1 or m is 1 and n is 0;

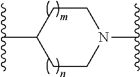

is pyrrolidin-3R-yl; -(L¹)ₐ-R³ is —C(O)-cyclopropyl;

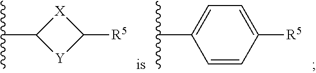

and b is 0, then R⁵ is other than 6-(4-methyl-piperazin-1-yl)-pyridin-3-yl.

In another embodiment, when R¹ and R² are taken together with the carbon atom to which they are bound to form tetrahydrofuran-3,3-diyl or tetrahydropyran-4,4-diyl, m is an integer from 0 to 1 and n is 0 or m is 0 and n is an integer from 0 to 1;

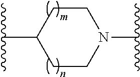

is selected from the group consisting of azetidin-3-yl, pyrrolidin-3R-yl, and pyrrolidin-3-yl; -(L¹)ₐ-R³ is selected from the group consisting of —C(O)—CF₃, —C(O)OCH₃, and —SO₂—CH₃,

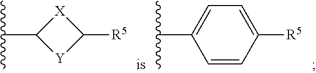

and b is 0, then R⁵ is 4-(1-methyl-pyrazol-4-yl)-phenyl.

In some embodiments, the compound has the structure of Formula (XXIX):

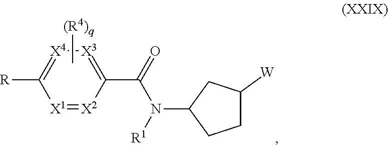

wherein R is Ar or Het, —C≡C—Ar or —C≡C—Het, W is furanyl, thiophenyl, pyrrolyl, pyrazolyl, oxazolyl, thiazolyl, triazolyl, oxadiazolyl or thiadiazolyl, each of which is unsubstituted or mono- or disubstituted by R², R¹ is A, [C(R³)₂]ₙAr¹, or [C(R³)₂]ₙCyc, R² is A, [C(R³)₂]ₙAr¹, Cyc or ═O; R⁴ is H, F, Cl, Br, OH, CN, NO₂. A′, OA′, SA, SO₂Me, COA′, CONH₂, CONHA′ or CONA′₂, each X¹, X², X³, X⁴, is, independently, CH or N, A is unbranched or branched alkyl with 1-10 C-atoms, wherein two adjacent carbon atoms may form a double bond and/or one or two non-adjacent CH— and/or CH₂— groups may be replaced by N-, O- and/or S-atoms and wherein 1-7H-atoms may be replaced by R⁵, Cyc is cycloalkyl with 3-7 C-atoms, which is unsubstituted or monosubstituted by OH, Hal or A, A′ is unbranched or branched alkyl with 1-6 C-atoms, wherein 1-5H-atoms may be replaced by F, R⁵ is F, Cl or OH, Ar is phenyl, which is unsubstituted or mono-, di-, tri-, tetra- or pentasubstituted by Hal, A, O[C(R³)₂]ₙHet¹, Ar¹, [C(R³)₂]ₚOR³, [C(R³)₂]ₚNR³₂, NO₂, CN, [C(R³)₂]ₚCOOR³, CONR³₂, [C(R³)₂]ₚNR³₂, NR³₂COA, NR³SO₂A, [C(R³)₂]ₚSO₂NR³₂, S(O)ₙA, O[C(R³)₂]ₘNR³₂, NHCOOA, NHCONR³₂ and/or COA, Ar¹ is phenyl or naphthyl, which is unsubstituted or mono-, di-, tri-, tetra- or pentasubstituted by Hal, A, [C(R³)₂]ₚOR³, [C(R³)₂]ₚNR³₂, NO₂, CN, [C(R³)₂]ₚCOOR³, [C(R³)₂]ₚNR³₂, NR³₂COA, NR³SO₂A, [C(R³)₂]ₚSO₂NR³₂, S(O)ₙA, O[C(R³)₂]ₘNR³₂, NHCOOA, NHCONR³₂ and/or COA, R³ is H or unbranched or branched alkyl with 1-6 C-atoms, Het is a mono- or bicyclic saturated, unsaturated or aromatic heterocycle having 1 to 4 N, O and/or S atoms, which is unsubstituted or mono-, di-, tri-, tetra- or pentasubstituted by Hal, A, [C(R³)₂]ₙOA [C(R³)₂]ₙNR³₂, SR³, NO₂, CN, COOR³, CONR³₂, COHet¹, NR³COA, NR³SO₂A, SO₂NR³₂, S(O)ₙA, O[C(R³)₂]ₘNR³₂, NHCOOA, NHCONR³₂, CHO, COA, ═S, ═NH, =NA and/or ═O (carbonyl oxygen), Hal is F, Cl, Br or I, m is 1, 2 or 3, n is 0, 1 or 2, p is 0, 1, 2, 3 or 4, q 0, 1, 2 or 3, with the proviso that only one or two of X¹, X², X³, X⁴ denote N, and pharmaceutically acceptable salts, tautomers and stereoisomers thereof, including mixtures thereof in all ratios.

In some embodiments, the compound is prepared wherein a compound of Formula (XXX):

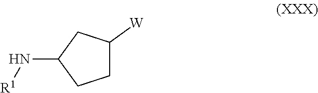

is reacted with a compound of Formula (XXXI):

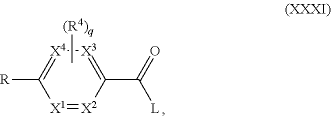

wherein L is Cl, Br, I, or a free or reactively functionally modified OH group, and/or a base or acid of Formula (XXIX) is converted into one of its salts.

In some embodiments, the compound of Formula (XXIX) is cis-configurated, such as in Formula (XXIX-A):

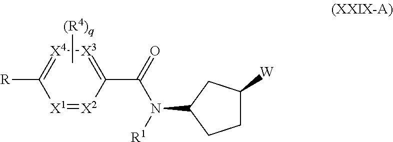

wherein the cyclopentane is preferably 1,3-cis-disubstituted.

Preferably, only one or two of X¹, X², X³, and X⁴ denote N. X¹ particularly preferably denotes C. X² particularly preferably denotes C. X³ particularly preferably denotes C or N. X⁴ preferably denotes C. In some embodiments, A denotes alkyl, wherein the alkyl is unbranched (linear) or branched, and has 1, 2, 3, 4, 5, 6, 7, 8, 9, or 10 C atoms. In some embodiments, A preferably denotes methyl, furthermore ethyl, propyl, isopropyl, butyl, isobutyl, sec-butyl or tert-butyl, furthermore also pentyl, 1-methylbutyl, 2-methylbutyl, 3-methylbutyl, 1,1-dimethylpropyl, 1,2-dimethylpropyl, 2,2-dimethylpropyl, 1-ethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1-ethyl-1-methylpropyl, 1-ethyl-2-methyl-propyl, 1,1,2-trimethylpropyl, 1,2,2-trimethylpropyl, or trifluoromethyl. In some embodiments, A preferably denotes unbranched or branched alkyl with 1-10 C atoms, wherein 1-7H atoms may be replaced by R⁵. In some embodiments, A is C₁₋₆ alkyl, e.g., methyl, ethyl, propyl, isopropyl, butyl, isobutyl, sec-butyl, tert-butyl, pentyl, hexyl, trifluoromethyl, pentafluoroethyl, or 1,1,1, -trifluoroethyl. In some embodiments, A is CH₂OCH₃, CH₂CH₂OH, or CH₂CH₂OCH₃. In some embodiments, Cyc is cyclopropyl, cyclobutyl, cyclopentyl, cyclohexyl, or cycloheptyl, optionally unsubstituted or monosubstituted by A. In some embodiments, A′ is alkyl, wherein the alkyl is unbranched (linear) or branched, and has 1, 2, 3, 4, 5 or 6 C atoms. A′ preferably denotes methyl, furthermore ethyl, propyl, isopropyl, butyl, isobutyl, sec-butyl or tert-butyl, furthermore also pentyl, 1-methylbutyl, 2-methylbutyl, 3-methylbutyl, 1,1-dimethylpropyl, 1,2-dimethylpropyl, 2,2-dimethylpropyl, 1-ethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1-ethyl-1-methylpropyl, 1-ethyl-2-methylpropyl, 1,1,2-trimethylpropyl, 1,2,2-trimethylpropyl, or trifluoromethyl. In some embodiments, A′ is C₁₋₆ alkyl. In some embodiments, R¹ is A. In some embodiments, R¹ is methyl. In some embodiments, R² is methyl, ethyl, propyl, isopropyl, butyl, cyclopropyl or 1-hydroxyethyl. In some embodiments, R³ is H, methyl, ethyl, propyl, isopropyl, butyl, pentyl or hexyl, particularly preferably H or methyl. In some embodiments, R⁴ is H or methoxy. In some embodiments, R⁵ is F, Cl or OH, particularly preferably OH. Ar denotes preferably o-tolyl, m-tolyl, p-tolyl, o-ethylphenyl, m-ethylphenyl, p-ethylphenyl, o-propylphenyl, m-propylphenyl, p-propylphenyl, o-isopropylphenyl, m-isopropylphenyl, p-isopropylphenyl, o-tert-butylphenyl, m-tert-butylphenyl, p-tert-butylphenyl, o-hydroxyphenyl, m-hydroxyphenyl, p-hydroxyphenyl, o-nitrophenyl, m-nitrophenyl, p-nitrophenyl, o-aminophenyl, m-aminophenyl, p-aminophenyl, o-(N-methylamino), m-(N-methylamino), p-(N-methylamino)phenyl, o-(N-methylaminocarbonyl)phenyl, m-(N-methylaminocarbonyl)phenyl, p-(N-methylaminocarbonyl)phenyl, o-methoxyphenyl, m-methoxyphenyl, p-methoxyphenyl, o-ethoxyphenyl, m-methoxyphenyl, p-ethoxyphenyl, o-ethoxycarbonyl-phenyl, m-ethoxycarbonyl-phenyl, p-ethoxycarbonyl-phenyl, o-(N,N-dimethylamino)phenyl, m-(N,N-dimethylamino)phenyl, p-(N,N-dimethylamino)phenyl, o-(N,N-dimethyl-aminocarbonyl)phenyl, m-(N,N-dimethyl-aminocarbonyl)phenyl, p-(N,N-dimethyl-aminocarbonyl)phenyl, o-(N-ethylamino)phenyl, m-(N-ethylamino)phenyl, p-(N-ethylamino)phenyl, o-(N,N-diethylamino)phenyl, m-(N,N-diethylamino)phenyl, p-(N,N-diethylamino)phenyl, o-fluorophenyl, m-fluorophenyl, p-fluorophenyl, o-bromophenyl, m-bromophenyl, p-bromophenyl, o-chlorophenyl, m-chlorophenyl, p-chlorophenyl, o-(methylsulfonamido)phenyl, m-(methylsulfonamido)phenyl, p-(methylsulfonamido)phenyl, o-(methyl-sulfonyl)phenyl, m-(methyl-sulfonyl)phenyl, p-(methyl-sulfonyl)phenyl, o-cyanophenyl, m-cyanophenyl, p-cyanophenyl, o-carboxyphenyl, m-carboxyphenyl, p-carboxyphenyl, o-methoxycarbonylphenyl, m-methoxycarbonylphenyl, p-methoxycarbonylphenyl, o-acetylphenyl, m-acetylphenyl, p-acetylphenyl, o-amino-sulfonylphenyl, m-amino-sulfonylphenyl, p-amino-sulfonylphenyl, o-[2-(morpholin-4-yl)ethoxy]phenyl, m-[2-(morpholin-4-yl)ethoxy]phenyl, p-[2-(morpholin-4-yl)ethoxy]phenyl, o-[3-(N,N-diethylamino)propoxy]phenyl, m-[3-(N,N-diethylamino)propoxy]phenyl, p-[3-(N,N-diethylamino)propoxy]phenyl, 2,3-difluorophenyl, 2,4-difluorophenyl, 2,5-difluorophenyl, 2,6-difluorophenyl, 3,4-difluorophenyl, 3,5-difluorophenyl, 2,3-dichlorophenyl, 2,4-dichlorophenyl, 2,5-dichlorophenyl, 2,6-dichlorophenyl, 3,4-dichlorophenyl, 3,5-dichlorophenyl, 2,3-dibromophenyl, 2,4-dibromophenyl, 2,5-dibromophenyl, 2,6-dibromophenyl, 3,4-dibromophenyl, 3,5-dibromophenyl, 2,4-dinitrophenyl, 2,5-dinitrophenyl, 2,5-dimethoxyphenyl, 3,4-dimethoxyphenyl, 3-nitro-4-chlorophenyl, 3-amino-4-chlorophenyl, 2-amino-3-chlorophenyl, 2-amino-4-chlorophenyl, 2-amino-5-chlorophenyl, 2-amino-6-chlorophenyl, 2-nitro-4-N,N-dimethylaminophenyl, 3-nitro-4-N,N-dimethylaminophenyl, 2,3-diaminophenyl, 2,3,4-trichlorophenyl, 2,3,5-trichlorophenyl, 2,3,6-trichlorophenyl, 2,4,6-trichlorophenyl, 3,4,5-trichlorophenyl, 2,4,6-trimethoxyphenyl, 2-hydroxy-3,5-dichlorophenyl, p-iodophenyl, 3,6-dichloro-4-aminophenyl, 4-fluoro-3-chlorophenyl, 2-fluoro-4-bromophenyl, 2,5-difluoro-4-bromophenyl, 3-bromo-6-methoxyphenyl, 3-chloro-6-methoxyphenyl, 3-chloro-4-acetamidophenyl, 3-fluoro-4-methoxyphenyl, 3-amino-6-methylphenyl, 3-chloro-4-acetamidophenyl or 2,5-dimethyl-4-chlorophenyl. In some embodiments, Ar is phenyl, which is unsubstituted or mono-, di-, tri-, tetra- or pentasubstituted by Hal and/or CN. In some embodiments, Ar is phenyl, which is unsubstituted or mono-, di-, or trisubstituted by Hal and/or CN. In some embodiments, Ar¹ is phenyl or naphthyl. In some embodiments, irrespective of further substitutions, Het denotes, for example, 2-furyl, 3-furyl, 2-thienyl, 3-thienyl, 1-pyrrolyl, 2-pyrrolyl, 3-pyrrolyl, 1-imidazolyl, 2-imidazolyl, 4-imidazolyl, 5-imidazolyl, 1-pyrazolyl, 3-pyrazolyl, 4-pyrazolyl, 5-pyrazolyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 3-isothiazolyl, 4-isothiazolyl, 5-isothiazolyl, 2-pyridyl, 3-pyridyl, 4-pyridyl, 2-pyrimidinyl, 4-pyrimidinyl, 5-pyrimidinyl, 6-pyrimidinyl, 1,2,3-triazol-1-yl, 1,2,3-triazol-4-yl, 1,2,3-triazol-5-yl, 1,2,4-triazol-1-yl, 1,2,4-triazol-3-yl, 1,2,4-triazol-5-yl, 1-tetrazolyl, 5-tetrazolyl, 1,2,3-oxadiazol-4-yl, 1,2,3-oxadiazol-5-yl, 1,2,4-oxadiazol-3-yl, 1,2,4-oxadiazol-5-yl, 1,3,4-thiadiazol-2-yl, 1,3,4-thiadiazol-5-yl, 1,2,4-thiadiazol-3-yl, 1,2,4-thiadiazol-5-yl, 1,2,3-thiadiazol-4-yl, 1,2,3-thiadiazol-5-yl, 3-pyridazinyl, 4-pyridazinyl, pyrazinyl, 1-indolyl, 2-indolyl, 3-indolyl, 4-indolyl, 5-indolyl, 6-indolyl, 7-indolyl, 4-isoindolyl, 5-isoindolyl, indazolyl, 1-benzimidazolyl, 2-benzimidazolyl, 4-benzimidazolyl, 5-benzimidazolyl, 1-benzopyrazolyl, 3-benzopyrazolyl, 4-benzopyrazolyl, 5-benzopyrazolyl, 6-benzopyrazolyl, 7-benzopyrazolyl, 2-benzoxazolyl, 4-benzoxazolyl, 5-benzoxazolyl, 6-benzoxazolyl, 7-benzoxazolyl, 3-benzisoxazolyl, 4-benzisoxazolyl, 5-benzisoxazolyl, 6-benzisoxazolyl, 7-benzisoxazolyl, 2-benzothiazolyl, 4-benzothiazolyl, 5-benzothiazolyl, 6-benzothiazolyl, 7-benzothiazolyl, 2-benzisothiazolyl, 4-benzisothiazolyl, 5-benzisothiazolyl, 6-benzisothiazolyl, 7-benzisothiazolyl, 4-benz-2,1,3-oxadiazolyl, 5-benz-2,1,3-oxadiazolyl, 6-benz-2,1,3-oxadiazolyl, 7-benz-2,1,3-oxadiazolyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-iso-quinolyl, 3-cinnolinyl, 4-cinnolinyl, 5-cinnolinyl, 6-cinnolinyl, 7-cinnolinyl, 8-cinnolinyl, 2-quinazolinyl, 4-quinazolinyl, 5-quinazolinyl, 6-quinazolinyl, 7-quinazolinyl, 8-quinazolinyl, 5-quinoxalinyl, 6-quinoxalinyl, 2-2H-benzo-1,4-oxazinyl, 3-2H-benzo-1,4-oxazinyl, 5-2H-benzo-1,4-oxazinyl, 6-2H-benzo-1,4-oxazinyl, 7-2H-benzo-1,4-oxazinyl, 8-2H-benzo-1,4-oxazinyl, 1,3-benzodioxol-5-yl, 1,4-benzodioxan-6-yl, 2,1,3-benzothiadiazol-4-yl, 2,1,3-benzothiadiazol-5-yl, 2,1,3-benzoxadiazol-5-yl, azabicyclo[3.2.1]octyl or dibenzofuranyl. The heterocyclic radicals may also be partially or fully hydrogenated.

In some embodiments, irrespective of further substitutions, Het can thus also denote, for example, 2,3-dihydro-2-furyl, 2,3-dihydro-3-furyl, 2,3-dihydro-4-furyl, 2,3-dihydro-5-furyl, 2,5-dihydro-2-furyl, 2,5-dihydro-3-furyl, 2,5-dihydro-4-furyl, 2,5-dihydro-5-furyl, tetrahydro-2-furyl, tetrahydro-3-furyl, 1,3-dioxolan-4-yl, tetrahydro-2-thienyl, tetrahydro-3-thienyl, 2,3-dihydro-1-pyrrolyl, 2,3-dihydro-2-pyrrolyl, 2,3-dihydro-3-pyrrolyl, 2,3-dihydro-4-pyrrolyl, 2,3-dihydro-5-pyrrolyl, 2,5-dihydro-1-pyrrolyl, 2,5-dihydro-2-pyrrolyl, 2,5-dihydro-3-pyrrolyl, 2,5-dihydro-4-pyrrolyl, 2,5-dihydro-5-pyrrolyl, 1-pyrrolidinyl, 2-pyrrolidinyl, 3-pyrrolidinyl, tetrahydro-1-imidazoyl, tetrahydro-2-imidazoyl, tetrahydro-4-imidazolyl, 2,3-dihydro-1-pyrazolyl, 2,3-dihydro-2-pyrazolyl, 2,3-dihydro-3-pyrazolyl, 2,3-dihydro-4-pyrazolyl, 2,3-dihydro-5-pyrazolyl, tetrahydro-1-pyrazolyl, tetrahydro-3-pyrazolyl, tetrahydro-4-pyrazolyl, 1,4-dihydro-1-pyridyl, 1,4-dihydro-2-pyridyl, 1,4-dihydro-3-pyridyl, 1,4-dihydro-4-pyridyl, 1,2,3,4-tetrahydro-1-pyridyl, 1,2,3,4-tetrahydro-2-pyridyl, 1,2,3,4-tetrahydro-3-pyridyl, 1,2,3,4-tetrahydro-4-pyridyl, 1,2,3,4-tetrahydro-5-pyridyl, 1,2,3,4-tetrahydro-6-pyridyl, 1-piperidinyl, 2-piperidinyl, 3-piperidinyl, 4-piperidinyl, 2-morpholinyl, 3-morpholinyl, 4-morpholinyl, tetrahydro-2-pyranyl, tetrahydro-3-pyranyl, tetrahydro-4-pyranyl, 1,4-dioxanyl, 1,3-dioxan-2-yl, 1,3-dioxan-4-yl, 1,3-dioxan-5-yl, hexahydro-1-pyridazinyl, hexahydro-3-pyridazinyl, hexahydro-4-pyridazinyl, hexahydro-1-pyrimidinyl, hexahydro-2-pyrimidinyl, hexahydro-4-pyrimidinyl, hexahydro-5-pyrimidinyl, 1-piperazinyl, 2-piperazinyl, 3-piperazinyl, 1,2,3,4-tetrahydro-1-quinolyl, 1,2,3,4-tetrahydro-2-quinolyl, 1,2,3,4-tetrahydro-3-quinolyl, 1,2,3,4-tetrahydro-4-quinolyl, 1,2,3,4-tetrahydro-5-quinolyl, 1,2,3,4-tetrahydro-6-quinolyl, 1,2,3,4-tetrahydro-7-quinolyl, 1,2,3,4-tetrahydro-8-quinolyl, 1,2,3,4-tetrahydro-1-isoquinolyl, 1,2,3,4-tetrahydro-2-isoquinolyl, 1,2,3,4-tetrahydro-3-isoquinolyl, 1,2,3,4-tetrahydro-4-isoquinolyl, 1,2,3,4-tetrahydro-5-isoquinolyl, 1,2,3,4-tetrahydro-6-isoquinolyl, 1,2,3,4-tetrahydro-7-isoquinolyl, 1,2,3,4-tetrahydro-8-isoquinolyl, 2-, 3-, 5-, 6-, 7- or 8-3,4-dihydro-2H-benzo-1,4-oxazinyl, furthermore preferably 2,3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydrobenzofuran-5-yl, 2,3-dihydrobenzofuran-6-yl, 2,3-(2-oxomethylenedioxy)phenyl, 3,4-dihydro-2H-1,5-benzodioxepin-6-yl, 3,4-dihydro-2H-1,5-benzodioxepin-7-yl, furthermore preferably 2,3-dihydrobenzofuranyl, 2,3-dihydro-2-oxofuranyl, 3,4-dihydro-2-oxo-1H-quinazolinyl, 2,3-dihydrobenzoxazolyl, 2-oxo-2,3-di-hydrobenzoxazolyl, 2,3-dihydrobenzimidazolyl, 1,3-dihydroindole, 2-oxo-1,3-dihydroindole, 2-oxo-2,3-dihydrobenzimidazolyl. In some embodiments, Het is a mono- or bicyclic aromatic heterocycle having 1 to 4 N, O and/or S atoms, which is unsubstituted or mono- or disubstituted by Hal and/or [C(R³)₂]ₙOA′. In some embodiments, Het is furyl, thienyl, pyrrolyl, imidazolyl, pyrazolyl, oxazolyl, isoxazolyl, thiazolyl, isothiazolyl, pyridyl, pyrimidinyl, triazolyl, tetrazolyl, oxadiazolyl, thiadiazolyl, pyridazinyl, pyrazinyl, benzoxazolyl, benzothiazolyl, benzimidazolyl, benzotriazolyl, indolyl, benzo-1,3-dioxolyl, benzodioxanyl, benzothiadiazolyl, indazolyl, benzofuranyl, quinolyl, isoquinolyl, pyrrolo[2,3-b]pyridinyl, oxazolo[5,4-b]pyridyl, imidazo[1,2-a]pyrimidinyl or oxazolo[5,4-c]pyridyl, each of which is unsubstituted or mono- or disubstituted by Hal and/or [C(R³)₂]ₙOA′. In some embodiments, Het is furyl, thienyl, pyrrolyl, imidazolyl, pyrazolyl, oxazolyl, isoxazolyl, thiazolyl, isothiazolyl, pyridyl, pyrimidinyl, triazolyl, pyrrolo[2,3-b]pyridinyl, imidazo[1,2-a]pyrimidinyl, benzoxazolyl, benzothiazolyl or benzimidazolyl, each of which is unsubstituted or mono- or disubstituted by Hal. In some embodiments, Het is a mono- or bicyclic aromatic heterocycle having 1 to 4 N, O and/or S atoms, which is unsubstituted or mono- or disubstituted by Hal. In some embodiments, Hal is F, Cl Br, or I.

Throughout the invention, all radicals which occur more than once may be identical or different, i.e. are independent of one another. In further embodiments, the compounds of the Formula I may have one or more chiral centers and can therefore occur in various stereoisomeric forms. The compounds of Formula I encompasses all these forms.

Accordingly, the invention relates, in particular, to the compounds of the Formula I in which at least one of the said radicals has one of the preferred meanings indicated above. Some preferred groups of compounds may be expressed by the following sub-formulae Ia to II, which conform to the formula I and in which the radicals not designated in greater detail have the meaning indicated for the formula I, but in which, X¹ is C, X² is C, X³ is C or N, X⁴ is C; In some embodiments, R¹ is A. In some embodiments, R² is A or Cyc. In some embodiments, R² is methyl, ethyl, propyl, isopropyl, butyl, cyclopropyl or 1-hydroxyethyl. In some embodiments, R⁴ is H or OA′. In some embodiments, R³ is H or methyl. In some embodiments, Ig A is unbranched or branched alkyl with 1-6 C-atoms, wherein 1-7H-atoms may be replaced by R⁵. In some embodiments, Ar is phenyl, which is unsubstituted or mono-, di-, tri-, tetra- or pentasubstituted by Hal and/or CN. In some embodiments, Het is a mono- or bicyclic aromatic heterocycle having 1 to 4 N, O and/or S atoms, which is unsubstituted or mono- or disubstituted by Hal and/or [C(R³)₂]ₙOA′. In some embodiments, Het is furyl, thienyl, pyrrolyl, imidazolyl, pyrazolyl, oxazolyl, isoxazolyl, thiazolyl, isothiazolyl, pyridyl, pyrimidinyl, triazolyl, tetrazolyl, oxadiazolyl, thiadiazolyl, pyridazinyl, pyrazinyl, benzoxazolyl, benzothiazolyl, benzimidazolyl, benzotriazolyl, indolyl, benzo-1,3-dioxolyl, benzodioxanyl, benzothiadiazolyl, indazolyl, benzofuranyl, quinolyl, isoquinolyl, pyrrolo[2,3-b]pyridinyl, oxazolo[5,4-b]pyridyl, imidazo[1,2-a]pyrimidinyl or oxazolo[5,4-c]pyridyl, each of which is unsubstituted or mono- or disubstituted by Hal and/or [C(R³)₂]ₙOA′. In some embodiments, R is Ar or Het, —C≡C—Ar or —C≡C-Het, W is furanyl, thiophenyl, pyrrolyl, pyrazolyl, oxazolyl, thiazolyl, triazolyl, oxadiazolyl or thiadiazolyl, each of which is unsubstituted or mono- or disubstituted by R², R¹ is A, R² is A or Cyc, R⁴ is H or OA′, X¹, X², X³, X⁴ each, independently of one another, denote CH or N, A is unbranched or branched alkyl with 1-10 C-atoms, wherein 1-7H-atoms may be replaced by R⁵, Cyc is cycloalkyl with 3-7 C-atoms, A′ is unbranched or branched alkyl with 1-6 C-atoms, R⁵ is OH, Ar is phenyl, which is unsubstituted or mono-, di-, tri-, tetra- or pentasubstituted by Hal and/or CN, Het is a mono- or bicyclic aromatic heterocycle having 1 to 4 N, O and/or S atoms, which is unsubstituted or mono- or disubstituted by Hal and/or [C(R³)₂]ₙOA′, Hal is F, Cl, Br or I, n is 0, 1 or 2, q is 0, 1, 2 or 3, with the proviso that only one or two of X¹, X², X³, X⁴ denote N; R is Ar or Het, —C═C—Ar or —C≡C-Het, W is furanyl, thiophenyl, pyrrolyl, pyrazolyl, oxazolyl, thiazolyl, triazolyl, oxadiazolyl or thiadiazolyl, each of which is unsubstituted or mono- or disubstituted by R², X¹ is C, X² is C, X³ is C or N, X⁴ is C, R¹ is methyl, R² is methyl, ethyl, propyl, isopropyl, butyl, cyclopropyl or 1-hydroxyethyl, R⁴ is H or methoxy, R⁵ is OH, Ar is phenyl, which is unsubstituted or mono-, di-, or trisubstituted by Hal and/or CN, Het is furyl, thienyl, pyrrolyl, imidazolyl, pyrazolyl, oxazolyl, isoxazolyl, thiazolyl, isothiazolyl, pyridyl, pyrimidinyl, triazolyl, pyrrolo[2,3-b]pyridinyl, imidazo[1,2-a]pyrimidinyl, benzoxazolyl, benzothiazolyl or benzimidazolyl, each of which is unsubstituted or mono- or disubstituted by Hal, Hal is F, Cl, Br or I, q 0, 1, 2 or 3, and pharmaceutically acceptable salts, tautomers and stereoisomers thereof, including mixtures thereof in all ratios.

In some embodiments, the compound is 4-benzoxazol-2-yl-N-methyl-N-[(1R,3S)-3-(5-propyl-[1,3,4]oxadiazol-2-yl)-cyclopentyl]-benzamide; biphenyl-4-carboxylic acid methyl-[(1R,3S)-3-(5-propyl-[1,3,4]oxadiazol-2-yl)-cyclopentyl-amide; 4-(4-fluoro-phenylethynyl)-N-methyl-N-[(1R,3S)-3-(5-propyl-[1,3,4]oxadiazol-2-yl)-cyclopentyl]-benzamide; 4′-cyano-biphenyl-4-carboxylic acid methyl-[(1R,3S)-3-(5-propyl-[1,3,4]oxadiazol-2-yl)-cyclopentyl]-amide; 4-benzoxazol-2-yl-N-[(1R,3S)-3-(5-ethyl-[1,3,4]oxadiazol-2-yl)-cyclopentyl]-N-methyl-benzamide; 4′-cyano-biphenyl-4-carboxylic acid methyl-[(1R,3S)-3-(5-ethyl-[1,3,4]oxadiazol-2-yl)-cyclopentyl]-amide; 4-(1H-benzimidazol-2-yl)-N-[(1R,3S)-3-(5-ethyl-[1,3,4]oxadiazol-2-yl)-cyclopentyl]-N-methyl-benzamide; N[(1R,3S)-3-(5-ethyl-[1,3,4]oxadiazol-2-yl)-cyclopentyl]-N-methyl-4-pyridin-4-yl-benzamide; 4-benzoxazol-2-yl-N-[(1R,3S)-3-(5-isopropyl-[1,3,4]oxadiazol-2-yl)-cyclopentyl]-N-methyl-benzamide; 4′-cyano-biphenyl-4-carboxylic acid methyl-[(1R,3S)-3-(5-isopropyl-[1,3,4]oxadiazol-2-yl)-cyclopentyl]-methyl-amide; 4-(1H-benzimidazol-2-yl)-N-[(1R,3S)-3-(5-isopropyl-[1,3,4]oxadiazol-2-yl)-cyclopentyl]-N-methyl-benzamide; N-[(1R,3S)-3-(5-isopropyl-[1,3,4]oxadiazol-2-yl)-cyclopentyl]-N-methyl-4-pyridin-4-yl-benzamide; 4-benzoxazol-2-yl-N-methyl-N-[(1R,3S)-3-(5-methyl-[1,3,4]oxadiazol-2-yl)-cyclopentyl]-benzamide; 4-benzothiazol-2-yl-N-methyl-N-[(1R,3S)-3-(5-methyl-[1,3,4]oxadiazol-2-yl)-cyclopentyl]-benzamide; N-methyl-N[(1R,3S)-3-(5-methyl-[1,3,4]oxadiazol-2-yl)-cyclopentyl]-4-pyridin-4-yl-benzamide; 4′-chloro-biphenyl-4-carboxylic acid methyl-[(1R,3S)-3-(5-methyl-[1,3,4]oxadiazol-2-yl)-cyclopentyl]-amide; 4-(1H-benzimidazol-2-yl)-N-methyl-N-[(1R,3S)-3-(5-methyl-[1,3,4]oxadiazol-2-yl(-cyclopetyl]-benzamide; 4-benzoxazol-2-yl-3-methoxy-N-methyl-N-[(1R,3S)-3-(5-methyl-[1,3,4]oxadiazol-2-yl)-cyclopentyl]-benzamide; 4-imidazo[1,2-a]pyrimidin-2-yl-N-methyl-N-[(1R,3S)-3-(5-methyl-[1,3,4]oxadiazol-2-yl)-cyclopentyl]-benzamide; 4-(4-chloro-phenylethynyl)-N-methyl-N-[(1R,3S)-3-(5-methyl-[1,3,4]oxadiazol-2-yl)-cyclopentyl]-benzamide; N-methyl-N-[(1R,3S)-3-(5-methyl-[1,3,4]oxadiazol-2-yl)-cyclopentyl]-4-pyridin-4-ylethynyl-benzamide; 5-benzoxazol-2-yl-pyridine-2-carboxylic acid methyl-[(1R,3S)-3-(5-methyl-[1,3,4]oxadiazol-2-yl)-cyclopentyl]-amide; biphenyl-4-carboxylic acid methyl-[(1R,3S)-3-(5-methyl-[1,3,4]oxadiazol-2-yl)-cyclopentyl]-amide; 4′-cyano-biphenyl-4-carboxylic acid methyl-[(1R,3S)-3-(5-methyl-[1,3,4]oxadiazol-2-yl)-cyclopentyl]-amide; 4-benzoxazol-2-yl-N-methyl-N-[(1R,3S)-3-(5-methyl-oxazol-2-yl)-cyclopentyl]-benzamide; 4-benzoxazol-2-yl-N-methyl-N-[(1R,3S)-3-(4-methyl-oxazol-2-yl)-cyclopentyl]-benzamide; j4-benzoxazol-2-yl-N-methyl-N-[(1R,3S)-3-(3-methyl-[1,2,4]oxadiazol-5-yl)-cyclopentyl]-benzamide; (rac)-cis-biphenyl-4-carboxylic acid [-3-(4-cyclopropyl-[1,2,3]triazol-1-yl)-cyclopentyl]-methyl-amide; (rac)-cis-biphenyl-4-carboxylic acid methyl-[3-(4-propyl-[1,2,3]triazol-1-yl)-cyclopentyl]-amide; or biphenyl-4-carboxylic acid{(1R,3S)-3-[4-((S)-1-hydroxy-ethyl)-[1,2,3]triazol-1-yl]-cyclopentyl}-methyl-amide.

In some embodiments, the compound is one of the following:

| | Compound |
|---|---|
| 1465 | 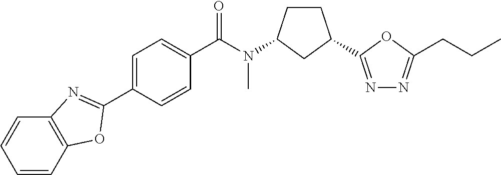 [SMILES: CCCc1nnc([C@H]2CC[C@@H](N(C)C(=O)c3ccc(-c4nc5ccccc5o4)cc3)C2)o1] |
| 1466 | 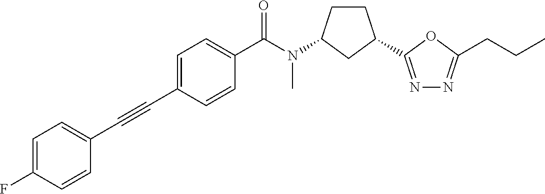 [SMILES: CCCc1nnc([C@H]2CC[C@@H](N(C)C(=O)c3ccc(C#Cc4ccc(F)cc4)cc3)C2)o1] |
| 1467 | 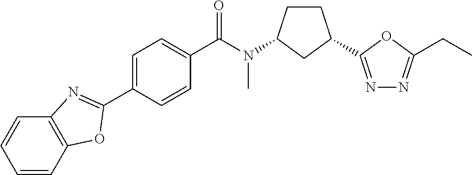 [SMILES: CCc1nnc([C@H]2CC[C@@H](N(C)C(=O)c3ccc(-c4nc5ccccc5o4)cc3)C2)o1] |
| 1468 | 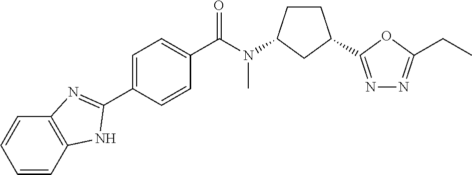 [SMILES: CCc1nnc([C@H]2CC[C@@H](N(C)C(=O)c3ccc(-c4nc5ccccc5[nH]4)cc3)C2)o1] |
| 1469 | 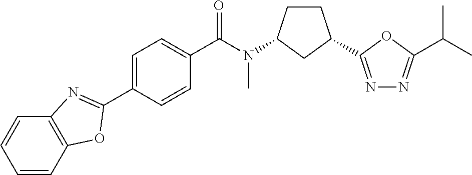 [SMILES: CC(C)c1nnc([C@H]2CC[C@@H](N(C)C(=O)c3ccc(-c4nc5ccccc5o4)cc3)C2)o1] |
| 1470 | 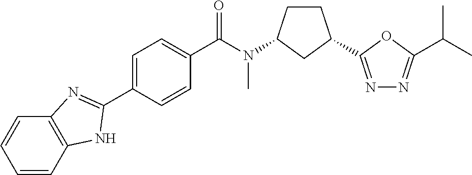 [SMILES: CC(C)c1nnc([C@H]2CC[C@@H](N(C)C(=O)c3ccc(-c4nc5ccccc5[nH]4)cc3)C2)o1] |
| 1471 | 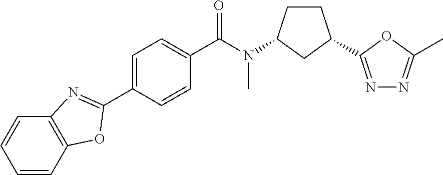 [SMILES: Cc1nnc([C@H]2CC[C@@H](N(C)C(=O)c3ccc(-c4nc5ccccc5o4)cc3)C2)o1] |
| 1472 | 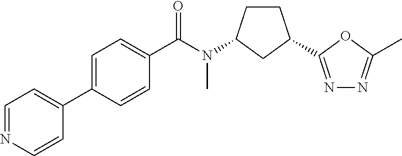 [SMILES: Cc1nnc([C@H]2CC[C@@H](N(C)C(=O)c3ccc(-c4ccncc4)cc3)C2)o1] |
| 1473 | 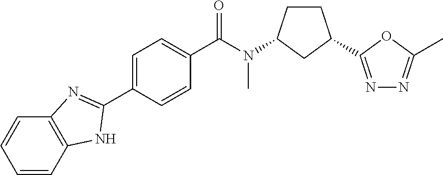 [SMILES: Cc1nnc([C@H]2CC[C@@H](N(C)C(=O)c3ccc(-c4nc5ccccc5[nH]4)cc3)C2)o1] |
| 1474 | 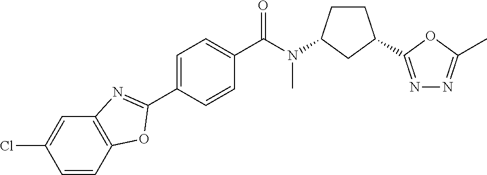 [SMILES: Cc1nnc([C@H]2CC[C@@H](N(C)C(=O)c3ccc(-c4nc5cc(Cl)ccc5o4)cc3)C2)o1] |
| 1475 | 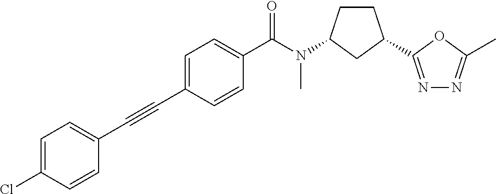 [SMILES: Cc1nnc([C@H]2CC[C@@H](N(C)C(=O)c3ccc(C#Cc4ccc(Cl)cc4)cc3)C2)o1] |
| 1476 | 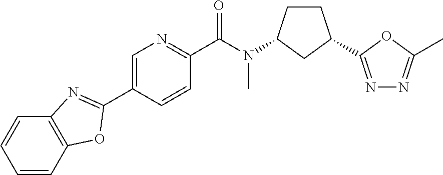 [SMILES: Cc1nnc([C@H]2CC[C@@H](N(C)C(=O)c3ccc(-c4nc5ccccc5o4)cn3)C2)o1] |
| 1477 | 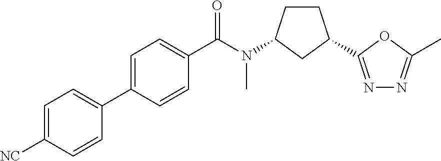 [SMILES: Cc1nnc([C@H]2CC[C@@H](N(C)C(=O)c3ccc(-c4ccc(C#N)cc4)cc3)C2)o1] |
| 1478 | 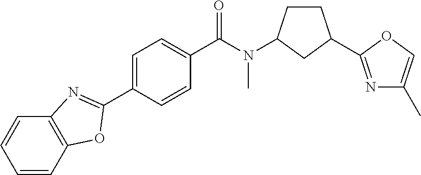 [SMILES: Cc1coc(C2CCC(N(C)C(=O)c3ccc(-c4nc5ccccc5o4)cc3)C2)n1] |
| 1479 | 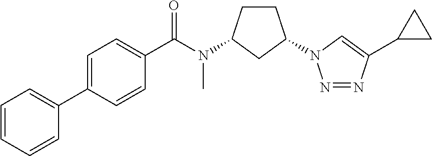 [SMILES: CN(C(=O)c1ccc(-c2ccccc2)cc1)[C@@H]1CC[C@H](n2cc(C3CC3)nn2)C1] |
| 1480 | 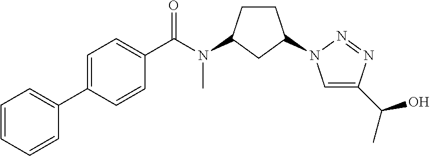 |
| 1481 | 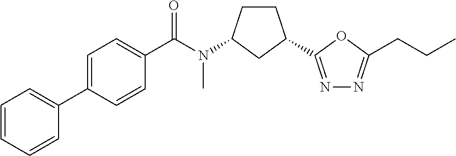 [SMILES: CCCc1nnc([C@H]2CC[C@@H](N(C)C(=O)c3ccc(-c4ccccc4)cc3)C2)o1] |
| 1482 | 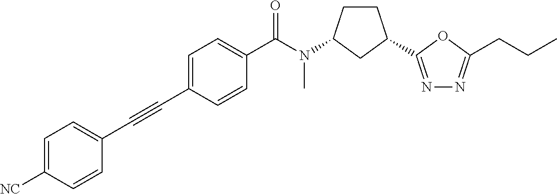 [SMILES: CCCc1nnc([C@H]2CC[C@@H](N(C)C(=O)c3ccc(C#Cc4ccc(C#N)cc4)cc3)C2)o1] |
| 1483 | 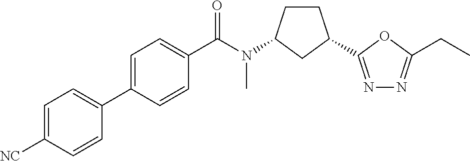 [SMILES: CCc1nnc([C@H]2CC[C@@H](N(C)C(=O)c3ccc(-c4ccc(C#N)cc4)cc3)C2)o1] |
| 1484 | 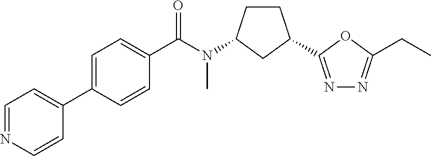 [SMILES: CCc1nnc([C@H]2CC[C@@H](N(C)C(=O)c3ccc(-c4ccncc4)cc3)C2)o1] |
| 1485 | 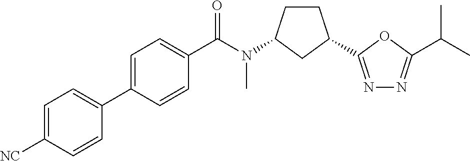 [SMILES: CC(C)c1nnc([C@H]2CC[C@@H](N(C)C(=O)c3ccc(-c4ccc(C#N)cc4)cc3)C2)o1] |
| 1486 | 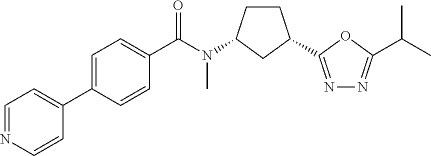 [SMILES: CC(C)c1nnc([C@H]2CC[C@@H](N(C)C(=O)c3ccc(-c4ccncc4)cc3)C2)o1] |
| 1487 | 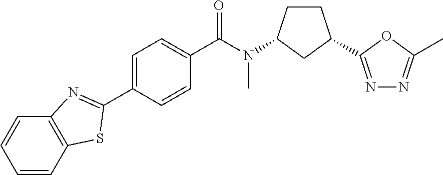 [SMILES: Cc1nnc([C@H]2CC[C@@H](N(C)C(=O)c3ccc(-c4nc5ccccc5s4)cc3)C2)o1] |
| 1488 | 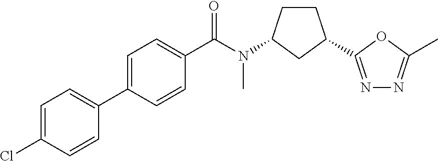 [SMILES: Cc1nnc([C@H]2CC[C@@H](N(C)C(=O)c3ccc(-c4ccc(Cl)cc4)cc3)C2)o1] |
| 1489 | 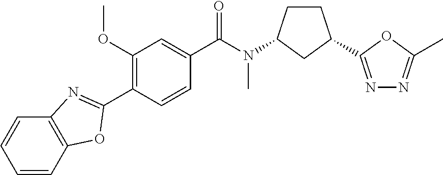 [SMILES: COc1cc(C(=O)N(C)[C@@H]2CC[C@H](c3nnc(C)o3)C2)ccc1-c1nc2ccccc2o1] |
| 1490 | 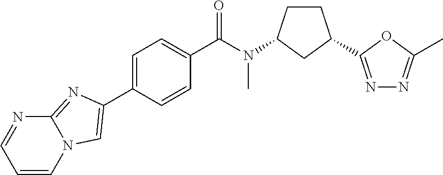 [SMILES: Cc1nnc([C@H]2CC[C@@H](N(C)C(=O)c3ccc(-c4cn5cccnc5n4)cc3)C2)o1] |
| 1491 | 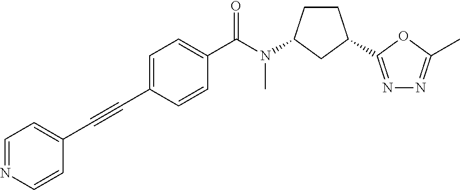 [SMILES: Cc1nnc([C@H]2CC[C@@H](N(C)C(=O)c3ccc(C#Cc4ccncc4)cc3)C2)o1] |
| 1492 | 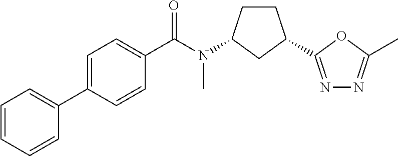 [SMILES: Cc1nnc([C@H]2CC[C@@H](N(C)C(=O)c3ccc(-c4ccccc4)cc3)C2)o1] |
| 1493 | 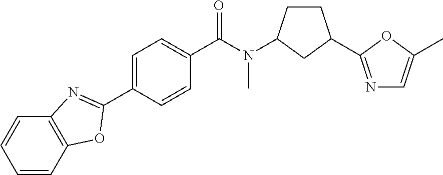 [SMILES: Cc1cnc(C2CCC(N(C)C(=O)c3ccc(-c4nc5ccccc5o4)cc3)C2)o1] |
| 1494 | 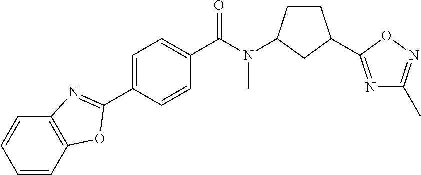 [SMILES: Cc1noc(C2CCC(N(C)C(=O)c3ccc(-c4nc5ccccc5o4)cc3)C2)n1] |
| 1495 | 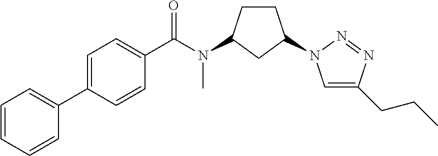 |

In some embodiments, the compound has the structure of Formula (XXXII):

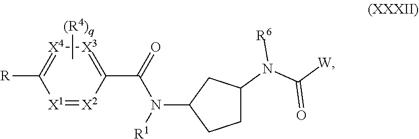

wherein R is Ar, Het, —C≡C—Ar or —C≡C—Het, W is NR²R^{2′}, Het¹, CH₂Het¹, A, Cyc, CH₂Cyc, Ar, CH₂Ar, [C(R³)₂]ₘNR⁶COA or [C(R³)₂]ₘCR³(COOA)NR⁶COA, R¹ is A, [C(R³)₂]ₙAr¹ or [C(R³)₂]ₙCyc, R², R² each, independently of one another, denote H, A or [C(R³)₂]ₙCyc, R⁴ is H, F, Cl, Br, OH, CN, NO₂, A′, OA′, SA′. SO₂Me, COA′, CONH₂, CONHA′ or CONA′2, R⁶ is H or A′, each of X¹, X², X³, X⁴ independently, is CH or N, A is unbranched or branched alkyl with 1-10 C-atoms, wherein two adjacent carbon atoms may form a double bond and/or one or two non-adjacent CH— and/or CH₂— groups may be replaced by N-, O- and/or S-atoms and wherein 1-7H-atoms may be replaced by R⁵, Cyc is cycloalkyl with 3-7 C-atoms, which is unsubstituted or monosubstituted by OH, Hal or A, A′ is unbranched or branched alkyl with 1-6 C-atoms, wherein 1-5H-atoms may be replaced by F, R⁵ is F, Cl or OH, Ar is phenyl, which is unsubstituted or mono-, di-, tri-, tetra- or pentasubstituted by Hal, A, O[C(R³)₂]ₙHet¹, Ar¹, [C(R³)₂]ₚOA, OCH₂Cyc, [C(R³)₂]ₚNR³₂, NO₂, CN, [C(R³)₂]_{P}COOR³, CONR³₂, [C(R³)₂]ₚNR³₂, NR³₂COA, NR³SO₂A, [C(R³)₂]_{P}SO₂NR³₂, S(O)ₙA, O[C(R³)₂]ₘNR³₂, NHCOOA, NHCONR³₂ and/or COA, Ar¹ is phenyl or naphthyl, which is unsubstituted or mono-, di-, tri-, tetra- or pentasubstituted by Hal, A, [C(R³)₂]ₚOR³, [C(R³)₂]ₚNR³₂) NO₂, CN, [C(R³)₂]ₚCOOR³, [C(R³)₂]ₚNR³₂, NR³₂COA, NR³SO₂A, [C(R³)₂]ₚSO₂NR³₂, S(O)ₙA, O[C(R³)₂]ₘNR³₂, NHCOOA, NHCONR³₂ and/or COA, R³ is H or unbranched or branched alkyl with 1-6 C-atoms, Het is a mono- or bicyclic saturated, unsaturated or aromatic heterocycle having 1 to 4 N, O and/or S atoms, which is unsubstituted or mono-, di-, tri-, tetra- or pentasubstituted by Hal, A, [C(R³)₂]ₙOA′, [C(R³)₂]ₙNR³₂, SR³, NO₂, [C(R³)₂]ₙCN, COOR³, Het¹, CONR³₂, COHet¹, NR³COA, NR³SO₂A, SO₂NR³₂, S(O)ₙA, O[C(R³)₂]ₘNR³₂, NHCOOA, NHCONR³₂, CHO, COA, ═S, ═NH, =NA and/or ═O (carbonyl oxygen), Het¹ is a mono- or bicyclic saturated, unsaturated or aromatic heterocycle having 1 to 4 N, O and/or S atoms, which is unsubstituted or mono-, di-, tri-, tetra- or pentasubstituted by Hal, A, [C(R³)₂]ₙOR³, [C(R³)₂]ₙNR³₂, SR³, NO₂, CN, COOR³, CONR³₂, NR³COA, NR³SO₂A, SO₂NR³₂, S(O)nA, O[C(R³)₂]ₘNR³₂, NHCOOA, NHCONR³₂, CHO, COA, ═S, ═NH, =NA and/or ═O (carbonyl oxygen), Hal is F, Cl, Br or I, m is 1, 2 or 3, n is 0, 1 or 2, p is 0, 1, 2, 3 or 4, q is 0, 1, 2 or 3, with the proviso that only one or two of X¹, X², X³, X⁴ denote N, and pharmaceutically acceptable salts, tautomers and stereoisomers thereof, including mixtures thereof in all ratios.

In some embodiments, the compound of Formula (XXXII) is prepared by a process wherein a compound of Formula (XXXIII):

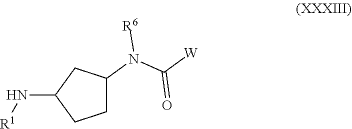

is reacted with a compound of Formula (XXXIV):

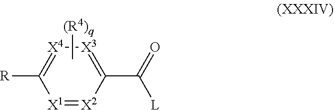

wherein L denotes Cl, Br, I, or a free or reactively functionally modified OH group, and/or a base or acid of formula (XXXII) is converted into one of its salts.

In some embodiments, the compound of Formula (XXXII) is cis-configurated, such that it has the structure of Formula (XXXII-A):

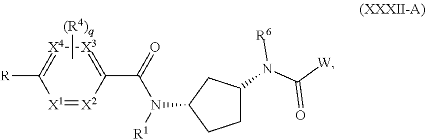

wherein the cyclopentane is preferably 1,3, -cis-disubstituted.

In some embodiments, A is alkyl, unbranched (linear) or branched, and has 1, 2, 3, 4, 5, 6, 7, 8, 9 or 10 C atoms. A preferably is methyl, furthermore ethyl, propyl, isopropyl, butyl, isobutyl, sec-butyl or tert-butyl, furthermore also pentyl, 1-methylbutyl, 2-methylbutyl, 3-methylbutyl, 1,1-dimethylpropyl, 1,2-dimethylpropyl, 2,2-dimethylpropyl, 1-ethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1-ethyl-1-methylpropyl, 1-ethyl-2-methyl-propyl, 1,1,2-trimethylpropyl, 1,2,2-trimethylpropyl, furthermore preferably, for example, trifluoromethyl. In some embodiments, A preferably is unbranched or branched alkyl with 1-10 C-atoms, wherein one or two non-adjacent CH— and/or CH₂— groups may be replaced by N- and/or O-atoms and wherein 1-7H-atoms may be replaced by R⁵ wherein 1-7H-atoms may be replaced by R⁵. In further embodiments, A is alkyl having 1, 2, 3, 4, 5 or 6 C atoms, preferably methyl, ethyl, propyl, isopropyl, butyl, isobutyl, sec-butyl, tert-butyl, pentyl, hexyl, trifluoromethyl, pentafluoroethyl or 1,1,1-trifluoroethyl. In some embodiments, A is preferably CH₂OCH₃, CH₂CH₂OH or CH₂CH₂OCH₃. Cyc is cyclopropyl, cyclobutyl, cyclopentyl, cyclohexyl or cycloheptyl, preferably unsubstituted or monosubstituted by OH, Hal or A. In some embodiments, A′ is alkyl, this is unbranched (linear) or branched, and has 1, 2, 3, 4, 5 or 6 C atoms. A preferably is methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3-, or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, furthermore preferably, for example, trifluoromethyl. In some embodiments, R² preferably is H. In some embodiments, R^{2′} preferably is H. In some embodiments, R³ preferably is H, methyl, ethyl, propyl, isopropyl, butyl, pentyl or hexyl, particularly preferably H or methyl. In some embodiments, R⁴ preferably is H, OA′, Hal or A′. In some embodiments, R⁵ preferably is F or Cl. In some embodiments, R⁶ preferably is H. In some embodiments, Ar is preferably o-, m- or p-tolyl, o-, m- or p-ethylphenyl, o-, m- or p-propylphenyl, o-, m- or p-isopropylphenyl, o-, m- or p-tert-butylphenyl, o-, m- or p-hydroxyphenyl, o-, m- or p-nitrophenyl, o-, m- or p-aminophenyl, o-, m- or p-(N-methylamino)phenyl, o-, m- or p-(N-methylaminocarbonyl)phenyl, o-, m- or p-methoxyphenyl, o-, m- or p-ethoxyphenyl, o-, m- or p-ethoxycarbonyl-phenyl, o-, m- or p-(N,N-dimethylamino)phenyl, o-, m- or p-(N,N-dimethyl-aminocarbonyl)phenyl, o-, m- or p-(N-ethylamino)phenyl, o-, m- or p-(N,N-diethylamino)phenyl, o-, m- or p-fluorophenyl, o-, m- or p-bromophenyl, o-, m- or p-chlorophenyl, o-, m- or p-(methylsulfonamido)phenyl, o-, m- or p-(methyl-sulfonyl)phenyl, o-, m- or p-cyanophenyl, o-, m- or p-carboxyphenyl, o-, m- or p-methoxycarbonylphenyl, o-, m- or p-acetylphenyl, o-, m- or p-amino-sulfonylphenyl, o-, m- or p-[2-(morpholin-4-yl)ethoxy]phenyl, o-, m- or p-[3-(N,N-diethylamino)propoxy]phenyl, furthermore preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,4- or 2,5-dinitrophenyl, 2,5- or 3,4-dimethoxyphenyl, 3-nitro-4-chlorophenyl, 3-amino-4-chloro-, 2-amino-3-chloro-, 2-amino-4-chloro-, 2-amino-5-chloro- or 2-amino-6-chlorophenyl, 2-nitro-4-N,N-dimethylamino- or 3-nitro-4-N,N-dimethylaminophenyl, 2,3-diaminophenyl, 2,3,4-, 2,3,5-, 2,3,6-, 2,4,6- or 3,4,5-trichlorophenyl, 2,4,6-trimethoxyphenyl, 2-hydroxy-3,5-dichlorophenyl, p-iodophenyl, 3,6-dichloro-4-aminophenyl, 4-fluoro-3-chlorophenyl, 2-fluoro-4-bromophenyl, 2,5-difluoro-4-bromophenyl, 3-bromo-6-methoxyphenyl, 3-chloro-6-methoxyphenyl, 3-chloro-4-acetamidophenyl, 3-fluoro-4-methoxyphenyl, 3-amino-6-methylphenyl, 3-chloro-4-acetamidophenyl or 2,5-dimethyl-4-chlorophenyl. In some embodiments, Ar furthermore preferably is phenyl, which is unsubstituted or mono-, di-, tri-, tetra- or pentasubstituted by Hal, CN, CONR³₂₁[C(R³)₂]ₚOA, [C(R³)₂]_{P}COOR³, A, Cyc and/or OCH₂Cyc. In some embodiments, Ar¹ preferably is phenyl or naphthyl. Irrespective of further substitutions, Het is, for example, 2- or 3-furyl, 2- or 3-thienyl, 1-, 2- or 3-pyrrolyl, 1-, 2,4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazoM-, -4- or -5-yl, 1,2,4-triazol-, -3- or 5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, indazolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-iso-quinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, further preferably 1,3-benzodioxol-5-yl, 1,4-benzodioxan-6-yl, 2,1,3-benzothiadiazol-4-, -5-yl or 2,1,3-benzoxadiazol-5-yl, azabicyclo[3.2.1]octyl or dibenzofuranyl. The heterocyclic radicals may also be partially or fully hydrogenated. In some embodiments, Het can thus also be, for example, 2,3-dihydro-2-, -3-, -4- or -5-furyl, 2,5-dihydro-2-, -3-, -4- or 5-furyl, tetrahydro-2- or -3-furyl, 1,3-dioxolan-4-yl, tetrahydro-2- or -3-thienyl, 2,3-dihydro-1-, -2-, -3-, -4- or -y-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-,-3-, -4-, -5-, -6-, -7- or -8-quinolyl, 1,2,3,4-tetrahydro-1-, -2-, -3-, -4-, -5-, -6-, -7- or -8-isoquinolyl, 2-, 3-, 5- , 6-, 7- or 8-3,4-dihydro-2H-benzo-1,4-oxazinyl, furthermore preferably 2,3-methylenedioxyphenyl, 3,4-methylenedioxyphenyl, 2,3-ethylenedioxyphenyl, 3,4-ethylenedioxy phenyl, 3,4-(difluoromethylenedioxy)phenyl, 2,3-dihydrobenzofuran-5- or 6-yl, 2,3-(2-oxomethylenedioxy)phenyl or also 3,4-dihydro-2H-1,5-benzodioxepin-6- or -7-yl, furthermore preferably 2,3-dihydrobenzofuranyl, 2,3-dihydro-2-oxofuranyl, 3,4-dihydro-2-oxo-1H-quinazolinyl, 2,3-dihydrobenzoxazolyl, 2-oxo-2,3-di-hydrobenzoxazolyl, 2,3-dihydrobenzimidazolyl, 1,3-dihydroindole, 2-oxo-1,3-dihydroindole or 2-oxo-2,3-dihydrobenzimidazolyl. In some embodiments, Het preferably is a mono- or bicyclic saturated, unsaturated or aromatic heterocycle having 1 to 4 N, O and/or S atoms, which is unsubstituted or mono-, di- or trisubstituted by Hal, [C(R³)₂]ₙOA′, [C(R³)₂]ₙNR³₂, CONR³₂, Het¹, A, [C(R³)₂]ₙCN and/or ═O. In further embodiments, Het is furyl, thienyl, pyrrolyl, imidazolyl, pyrazolyl, oxazolyl, isoxazolyl, thiazolyl, isothiazolyl, pyridyl, pyrimidinyl, triazolyl, tetrazolyl, oxadiazolyl, thiadiazolyl, pyridazinyl, pyrazinyl, benzoxazolyl, benzothiazolyl, benzimidazolyl, benzotriazolyl, indolyl, benzo-1,3-dioxolyl, benzodioxanyl, benzothiadiazolyl, indazolyl, benzofuranyl, quinolyl, isoquinolyl, oxazolo[5,4-b]pyridyl, imidazo[1,2-a]pyrimidinyl, imidazo[1,2-a]pyridyl, oxazolo[5,4-c]pyridyl, 2,3-dihydro-indolyl, 2,3-dihydro-benzo[1,4]dioxinyl, tetrahydropyranyl, 2,3-dihydro-benzimidazolyl, pyrrolo[2,3-c]pyridyl, oxazolo[4,5-b]pyridyl, furo[3,2-b]pyridyl or pyrrolo[3,2-b]pyridyl, each of which is unsubstituted or mono-, di- or trisubstituted by Hal, [C(R³)₂]ₙOA′, [C(R³)₂]ₙNR³₂, CONR³₂, A, CN and/or ═O. In some embodiments, Het furthermore preferably is furyl, thienyl, pyrrolyl, imidazolyl, pyrazolyl, oxazolyl, isoxazolyl, thiazolyl, isothiazolyl, pyridyl, pyrimidinyl, imidazo[1,2-a]pyrimidinyl, benzoxazolyl, benzothiazolyl, benzimidazolyl, 2,3-dihydro-indolyl, 2,3-dihydro-benzo[1,4]dioxinyl, tetrahydropyranyl, 2,3-dihydro-benzimidazolyl, pyrrolo[2,3-c]pyridyl, oxazolo[4,5-b]pyridyl, furo[3,2-b]pyridyl or pyrrolo[3,2-b]pyridyl, each of which is unsubstituted or mono-, di- or trisubstituted by Hal, [C(R³)₂]ₙOA [C(R³)₂]ₙNR³₂) CONR³₂, A, CN and/or ═O. In some embodiments, Het¹ is, for example, 2- or 3-furyl, 2- or 3-thienyl, 1-, 2- or 3-pyrrolyl, 1-, 2, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazoM-, -3- or 5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, indazolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-iso-quinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, further preferably 1,3-benzodioxol-5-yl, 1,4-benzodioxan-6-yl, 2,1,3-benzothiadiazol-4-, -5-yl or 2,1,3-benzoxadiazol-5-yl, azabicyclo[3.2.1]octyl or dibenzofuranyl. The heterocyclic radicals may also be partially or fully hydrogenated. In further embodiments, Het can thus also denote, for example, 2,3-dihydro-2-, -3-, -4- or -5-furyl, 2,5-dihydro-2-, -3-, -4- or 5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2, 3,4-tetrahydro-1-, -2-, -3-, -4-, -5-, -6-, -7- or -8-quinolyl, 1,2,3,4-tetrahydro-1-, -2-, -3-, -4-, 0.5-, -6-, -7- or -8-isoquinolyl, 2-, 3-, 5-, 6-, 7- or 8-3,4-dihydro-2H-benzo-1,4-oxazinyl, furthermore preferably 2,3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydrobenzofuran-5- or 6-yl, 2,3-(2-oxomethylenedioxy)phenyl or also 3,4-dihydro-2H-1,5-benzodioxepin-6- or -7-yl, furthermore preferably 2,3-dihydrobenzofuranyl, 2,3-dihydro-2-oxofuranyl, 3,4-dihydro-2-oxo-1H-quinazolinyl, 2,3-dihydrobenzoxazolyl, 2-oxo-2,3-di-hydrobenzoxazolyl, 2,3-dihydrobenzimidazolyl, 1,3-dihydroindole, 2-oxo-1,3-dihydroindole or 2-oxo-2,3-dihydrobenzimidazolyl.

In some embodiments, Het¹ preferably is a monocyclic saturated, unsaturated or aromatic heterocycle having 1 to 4 N, O and/or S atoms, which is unsubstituted or mono- or disubstituted by Hal, A and/or ═O. In further embodiments, Het¹ is furyl, thienyl, pyrrolyl, imidazolyl, pyrazolyl, oxazolyl, isoxazolyl, thiazolyl, isothiazolyl, pyridyl, pyrimidinyl, tri-azolyl, tetrazolyl, oxadiazolyl, thiadiazolyl, pyridazinyl, pyrazinyl, tetrahydrofuranyl, tetrahydropyranyl, pyrrolidinyl, piperidinyl or morpholinyl, each of which is unsubstituted or mono- or disubstituted by Hal, A and/or ═O.

In some embodiments, Hal is F, Cl or Br, but also I, particularly preferably F or Cl.

Throughout the invention, all radicals which occur more than once may be identical or different, i.e. are independent of one another.

The compounds of the Formula (XXXII) may have one or more chiral centres and can therefore occur in various stereoisomeric forms. The Formula (XXXII) encompasses all these forms.

Accordingly, the invention relates, in particular, to the compounds of the Formula (XXXII) in which at least one of the said radicals has one of the preferred meanings indicated above. Some preferred groups of compounds may be expressed by the following sub-formulae (XXXII-A) to (I-Q), which conform to the Formula (XXXII) and in which the radicals not designated in greater detail have the meaning indicated for the Formula (XXXII), but in which in Formula (XXXII-A) X¹, X², X⁴ denote CH, and X³ is N; in Formula (XXXII-B) X¹, X², X³, X⁴ denote CH, in Formula (XXXII-C) X¹, X³, X⁴ denote CH, X² is N; in Formula (XXXII-D) X¹, X², X³ denote CH, X⁴ is N; in Formula (I-E) X¹, X² denote CH, X³, X⁴ denote N; in Formula (XXXII-F) X³, X⁴ denote CH, X¹, X² denote N; in Formula (XXXII-G) R² is H; in Formula (XXXII-H) R² is H; in Formula (XXXII-I) R⁴ is H, OA′, Hal or A′; in Formula (XXXII-J) R³ is H or methyl; in Formula (XXXII-K) A is unbranched or branched alkyl with 1-10 C-atoms, wherein one or two non-adjacent CH— and/or CH₂— groups may be replaced by N- and/or O-atoms and wherein 1-7H— atoms may be replaced by R⁵; in Formula (XXXII-L) Ar is phenyl, which is unsubstituted or mono-, di-, tri-, tetra- or pentasubstituted by Hal, CN, CONR³₂, [C(R³)₂]_{P}OA, [C(R³)₂]_{P}COOR³, A, Cyc and/or OCH₂Cyc; in Formula (XXXII-M) Het or bicyclic saturated, unsaturated or aromatic heterocycle having 1 to 4 N, O and/or S atoms, mono-, di- or trisubstituted by Hal, [C(R³)₂]ₙOA [C(R³)₂]ₙNR³₂, CONR³₂, Het¹, A, [C(R³)₂]ₙCN and/or ═O; in Formula (XXXII-N) Het is furyl, thienyl, pyrrolyl, imidazolyl, pyrazolyl, oxazolyl, isoxazolyl, thiazolyl, isothiazolyl, pyridyl, pyrimidinyl, triazolyl, tetrazolyl, oxadiazolyl, thiadiazolyl, pyridazinyl, pyrazinyl, benzoxazolyl, benzothiazolyl, benzimidazolyl, benzotriazolyl, indolyl, benzo-1,3-dioxolyl, benzodioxanyl, benzothiadiazolyl, indazolyl, benzofuranyl, quinolyl, isoquinolyl, oxazolo[5,4-b]pyridyl, imidazo[1,2-a]pyrimidinyl, imidazo[1,2-a]pyridyl, oxazolo[5,4-c]pyridyl, 2,3-dihydro-indolyl, 2,3-dihydro-benzo[1,4]dioxinyl, tetrahydropyranyl, 2,3-dihydro-benzimidazolyl, pyrrolo[2,3-c]pyridyl, oxazolo[4,5-b]pyridyl, furo[3,2-b]pyridyl or pyrrolo[3,2-b]pyridyl, each of which is unsubstituted or mono-, di- or trisubstituted by Hal, [C(R³)₂]ₙOA′, [C(R³)₂]ₙNR³₂, CONR³₂, Het¹, A, [C(R³)₂]ₙCN and/or ═O; in Formula (XXXII-O) Het¹ is a monocyclic saturated, unsaturated or aromatic heterocycle having 1 to 4 N, O and/or S atoms, which is unsubstituted or mono- or disubstituted by Hal, A and/or ═O; in Formula (XXXII-P) Het¹ is furyl, thienyl, pyrrolyl, imidazolyl, pyrazolyl, oxazolyl, isoxazolyl, thiazolyl, isothiazolyl, pyridyl, pyrimidinyl, triazolyl, tetrazolyl, oxadiazolyl, thiadiazolyl, pyridazinyl, pyrazinyl, tetrahydrofuranyl, tetrahydropyranyl, pyrrolidinyl, piperidinyl or morpholinyl, each of which is unsubstituted or mono- or disubstituted by Hal, A and/or ═O; in Formula (XXXII-Q) R is Ar; Het, —C≡C—Ar or —C≡C-Het; W is NR²R², Het¹, CH₂Het¹, A, Cyc, CH₂Cyc, Ar, CH₂Ar, [C(R³)₂]ₘNR⁶COA or [C(R³)₂]ₘCR³(COOA)NR⁶COA; R¹ is A; R³ is H or unbranched or branched alkyl with 1-6 C— atoms; R⁴ is H, OA′, Hal or A′, X¹, X², X³, X⁴ each, independently of one another, denote CH or N; A is unbranched or branched alkyl with 1-10 C-atoms, wherein one or two non-adjacent CH— and/or CH₂— groups may be replaced by N- and/or O-atoms and wherein 1-7H-atoms may be replaced by R⁵, is cycloalkyl with 3-7 C-atoms, which is unsubstituted or monosubstituted by A′; A′ is unbranched or branched alkyl with 1-6 C-atoms, wherein 1-5H-atoms may be replaced by F; R⁵ is F or Cl; R⁶ is H or A′; Ar is phenyl, which is unsubstituted or mono-, di-, tri-, tetra- or pentasubstituted by Hal, CN, CONR³₂, [C(R³)₂]_{P}OA, [C(R³)₂]ₚCOOR³, A, Cyc and/or OCH₂Cyc, Het is furyl, thienyl, pyrrolyl, imidazolyl, pyrazolyl, oxazolyl, isoxazolyl, thiazolyl, isothiazolyl, pyridyl, pyrimidinyl, triazolyl, tetrazolyl, oxadiazolyl, thiadiazolyl, pyridazinyl, pyrazinyl, benzoxazolyl, benzothiazolyl, benzimidazolyl, benzotriazolyl, indolyl, benzo-1,3-dioxolyl, benzodioxanyl, benzothiadiazolyl, indazolyl, benzofuranyl, quinolyl, isoquinolyl, oxazolo[5,4-b]pyridyl, imidazo[1,2-a]pyrimidinyl, imidazo[1,2-a]pyridyl, oxazolo[5,4-c]pyridyl, 2,3-dihydro-indolyl, 2,3-dihydro-benzo[1,4]dioxinyl, tetrahydropyranyl, 2,3-dihydro-benzimidazolyl, pyrrolo[2,3-c]pyridyl, oxazolo[4,5-b]pyridyl, furo[3,2-b]pyridyl or pyrrolo[3,2-b]pyridyl, each of which is unsubstituted or mono-, di- or trisubstituted by Hal, [C(R³)₂]ₙOA′, [C(R³)₂]ₙNR³₂, CONR³₂, Het¹, A, [C(R³)₂]ₙCN and/or ═O; Het¹ is furyl, thienyl, pyrrolyl, imidazolyl, pyrazolyl, oxazolyl, isoxazolyl, thiazolyl, isothiazolyl, pyridyl, pyrimidinyl, triazolyl, tetrazolyl, oxadiazolyl, thiadiazolyl, pyridazinyl, pyrazinyl, tetrahydrofuranyl, tetrahydropyranyl, pyrrolidinyl, piperidinyl or morpholinyl, each of which is unsubstituted or mono- or disubstituted by Hal, A and/or ═O; Hal is F, Cl, Br or I; m is 1, 2 or 3; n is 0, 1 or 2; p is 0, 1, 2, 3 or 4; q is 0, 1, 2 or 3; with the proviso that only one or two of X¹, X², X³, X⁴ denote N, and pharmaceutically acceptable salts, tautomers and stereoisomers thereof, including mixtures thereof in all ratios.

In some embodiments, the compound is one of the following:

| | Compound |
|---|---|
| 1496 | 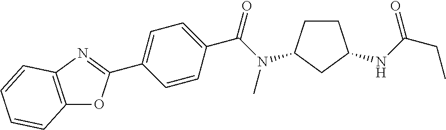 [SMILES: CCC(=O)N[C@H]1CC[C@@H](N(C)C(=O)c2ccc(-c3nc4ccccc4o3)cc2)C1] |
| 1497 | 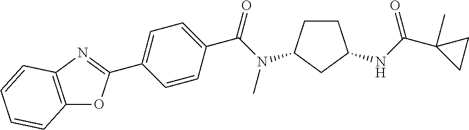 [SMILES: CN(C(=O)c1ccc(-c2nc3ccccc3o2)cc1)[C@@H]1CC[C@H](NC(=O)C2(C)CC2)C1] |
| 1498 | 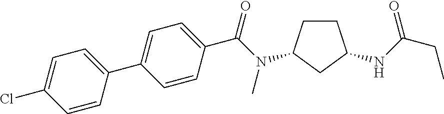 [SMILES: CCC(=O)N[C@H]1CC[C@@H](N(C)C(=O)c2ccc(-c3ccc(Cl)cc3)cc2)C1] |
| 1499 | 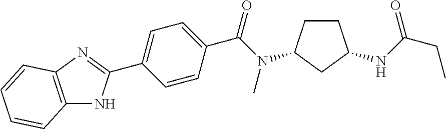 [SMILES: CCC(=O)N[C@H]1CC[C@@H](N(C)C(=O)c2ccc(-c3nc4ccccc4[nH]3)cc2)C1] |
| 1500 | 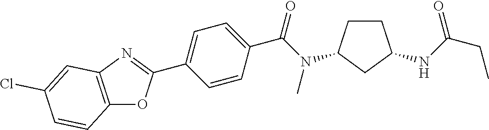 [SMILES: CCC(=O)N[C@H]1CC[C@@H](N(C)C(=O)c2ccc(-c3nc4cc(Cl)ccc4o3)cc2)C1] |
| 1501 | 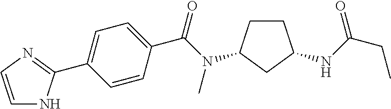 [SMILES: CCC(=O)N[C@H]1CC[C@@H](N(C)C(=O)c2ccc(-c3ncc[nH]3)cc2)C1] |
| 1502 | 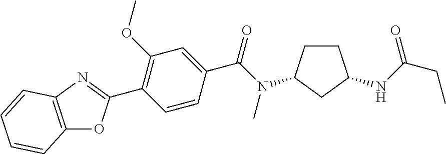 [SMILES: CCC(=O)N[C@H]1CC[C@@H](N(C)C(=O)c2ccc(-c3nc4ccccc4o3)c(OC)c2)C1] |
| 1503 | 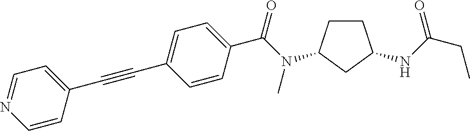 [SMILES: CCC(=O)N[C@H]1CC[C@@H](N(C)C(=O)c2ccc(C#Cc3ccncc3)cc2)C1] |
| 1504 | 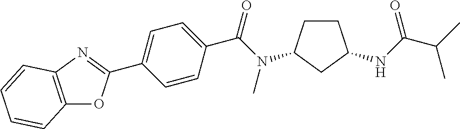 [SMILES: CC(C)C(=O)N[C@H]1CC[C@@H](N(C)C(=O)c2ccc(-c3nc4ccccc4o3)cc2)C1] |
| 1505 | 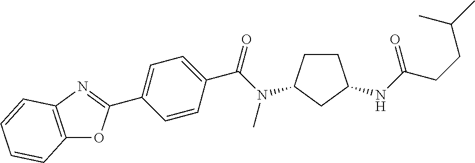 [SMILES: CC(C)CCC(=O)N[C@H]1CC[C@@H](N(C)C(=O)c2ccc(-c3nc4ccccc4o3)cc2)C1] |
| 1506 | 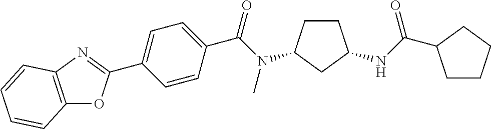 [SMILES: CN(C(=O)c1ccc(-c2nc3ccccc3o2)cc1)[C@@H]1CC[C@H](NC(=O)C2CCCC2)C1] |
| 1507 | 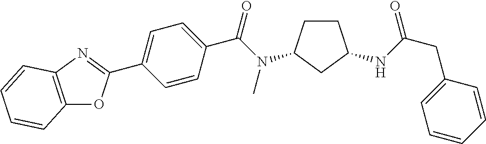 [SMILES: CN(C(=O)c1ccc(-c2nc3ccccc3o2)cc1)[C@@H]1CC[C@H](NC(=O)Cc2ccccc2)C1] |
| 1508 | 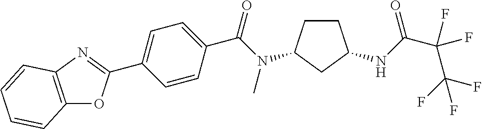 [SMILES: CN(C(=O)c1ccc(-c2nc3ccccc3o2)cc1)[C@@H]1CC[C@H](NC(=O)C(F)(F)C(F)(F)F)C1] |
| 1509 | 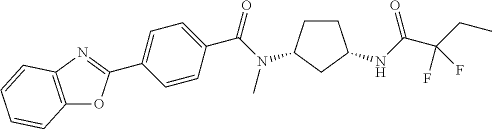 [SMILES: CCC(F)(F)C(=O)N[C@H]1CC[C@@H](N(C)C(=O)c2ccc(-c3nc4ccccc4o3)cc2)C1] |
| 1510 | 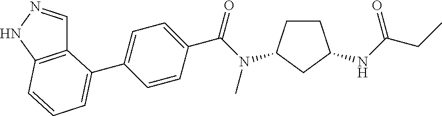 [SMILES: CCC(=O)N[C@H]1CC[C@@H](N(C)C(=O)c2ccc(-c3cccc4[nH]ncc34)cc2)C1] |
| 1511 | 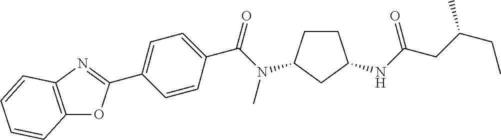 [SMILES: CC[C@@H](C)CC(=O)N[C@H]1CC[C@@H](N(C)C(=O)c2ccc(-c3nc4ccccc4o3)cc2)C1] |
| 1512 | 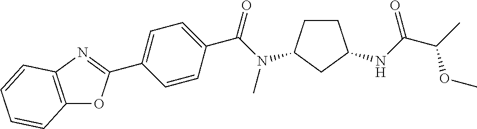 [SMILES: CO[C@@H](C)C(=O)N[C@H]1CC[C@@H](N(C)C(=O)c2ccc(-c3nc4ccccc4o3)cc2)C1] |
| 1513 | 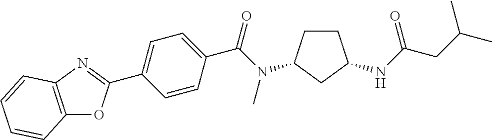 [SMILES: CC(C)CC(=O)N[C@H]1CC[C@@H](N(C)C(=O)c2ccc(-c3nc4ccccc4o3)cc2)C1] |
| 1514 | 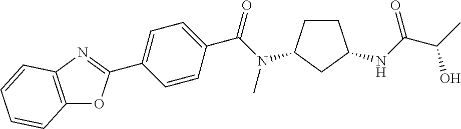 [SMILES: C[C@H](O)C(=O)N[C@H]1CC[C@@H](N(C)C(=O)c2ccc(-c3nc4ccccc4o3)cc2)C1] |
| 1515 | 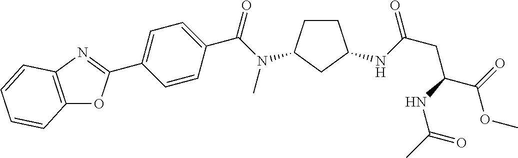 [SMILES: COC(=O)[C@H](CC(=O)N[C@H]1CC[C@@H](N(C)C(=O)c2ccc(-c3nc4ccccc4o3)cc2)C1)NC(C)=O] |
| 1516 | 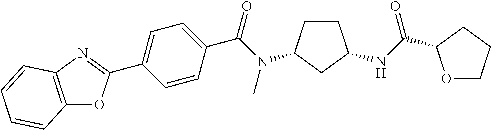 [SMILES: CN(C(=O)c1ccc(-c2nc3ccccc3o2)cc1)[C@@H]1CC[C@H](NC(=O)[C@@H]2CCCO2)C1] |
| 1517 | 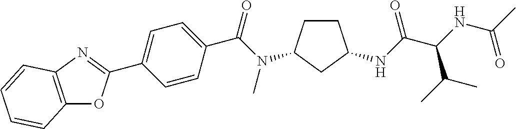 [SMILES: CC(=O)N[C@H](C(=O)N[C@H]1CC[C@@H](N(C)C(=O)c2ccc(-c3nc4ccccc4o3)cc2)C1)C(C)C] |
| 1518 | 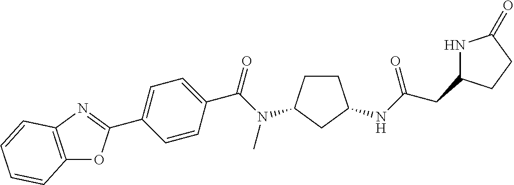 |
| 1519 | 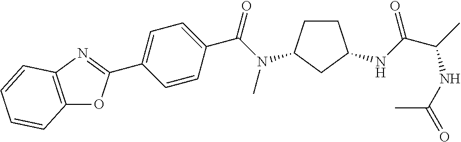 [SMILES: CC(=O)N[C@@H](C)C(=O)N[C@H]1CC[C@@H](N(C)C(=O)c2ccc(-c3nc4ccccc4o3)cc2)C1] |
| 1520 | 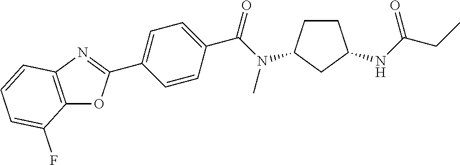 [SMILES: CCC(=O)N[C@H]1CC[C@@H](N(C)C(=O)c2ccc(-c3nc4cccc(F)c4o3)cc2)C1] |
| 1521 | 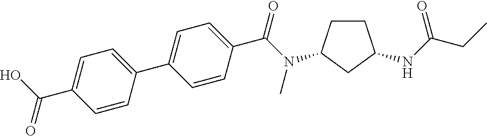 [SMILES: CCC(=O)N[C@H]1CC[C@@H](N(C)C(=O)c2ccc(-c3ccc(C(=O)O)cc3)cc2)C1] |
| 1522 | 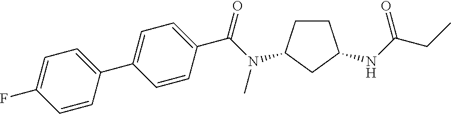 [SMILES: CCC(=O)N[C@H]1CC[C@@H](N(C)C(=O)c2ccc(-c3ccc(F)cc3)cc2)C1] |
| 1523 | 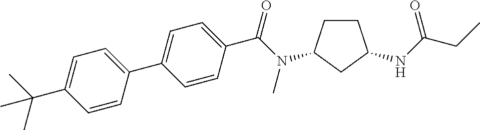 [SMILES: CCC(=O)N[C@H]1CC[C@@H](N(C)C(=O)c2ccc(-c3ccc(C(C)(C)C)cc3)cc2)C1] |
| 1524 | 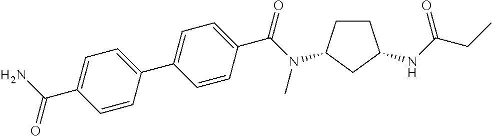 [SMILES: CCC(=O)N[C@H]1CC[C@@H](N(C)C(=O)c2ccc(-c3ccc(C(N)=O)cc3)cc2)C1] |
| 1525 | 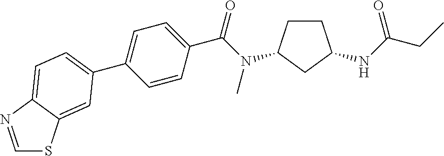 [SMILES: CCC(=O)N[C@H]1CC[C@@H](N(C)C(=O)c2ccc(-c3ccc4ncsc4c3)cc2)C1] |
| 1526 | 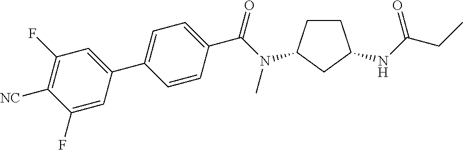 [SMILES: CCC(=O)N[C@H]1CC[C@@H](N(C)C(=O)c2ccc(-c3cc(F)c(C#N)c(F)c3)cc2)C1] |
| 1527 | 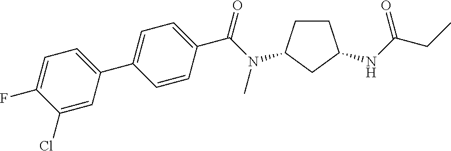 [SMILES: CCC(=O)N[C@H]1CC[C@@H](N(C)C(=O)c2ccc(-c3ccc(F)c(Cl)c3)cc2)C1] |
| 1528 | 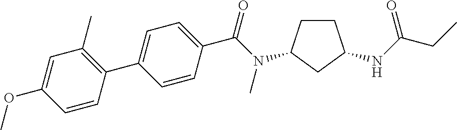 [SMILES: CCC(=O)N[C@H]1CC[C@@H](N(C)C(=O)c2ccc(-c3ccc(OC)cc3C)cc2)C1] |
| 1529 | 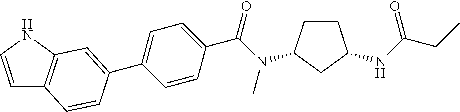 [SMILES: CCC(=O)N[C@H]1CC[C@@H](N(C)C(=O)c2ccc(-c3ccc4cc[nH]c4c3)cc2)C1] |
| 1530 | 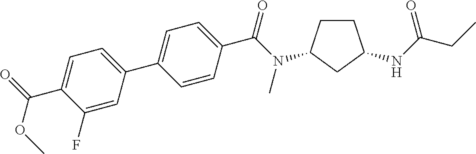 [SMILES: CCC(=O)N[C@H]1CC[C@@H](N(C)C(=O)c2ccc(-c3ccc(C(=O)OC)c(F)c3)cc2)C1] |
| 1531 | 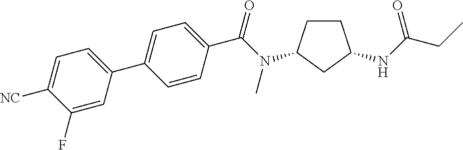 [SMILES: CCC(=O)N[C@H]1CC[C@@H](N(C)C(=O)c2ccc(-c3ccc(C#N)c(F)c3)cc2)C1] |
| 1532 | 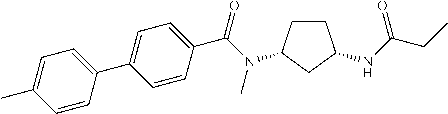 [SMILES: CCC(=O)N[C@H]1CC[C@@H](N(C)C(=O)c2ccc(-c3ccc(C)cc3)cc2)C1] |
| 1533 | 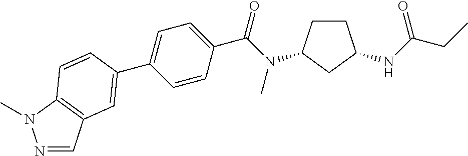 [SMILES: CCC(=O)N[C@H]1CC[C@@H](N(C)C(=O)c2ccc(-c3ccc4c(cnn4C)c3)cc2)C1] |
| 1534 | 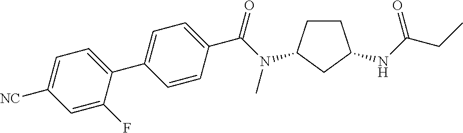 [SMILES: CCC(=O)N[C@H]1CC[C@@H](N(C)C(=O)c2ccc(-c3ccc(C#N)cc3F)cc2)C1] |
| 1535 | 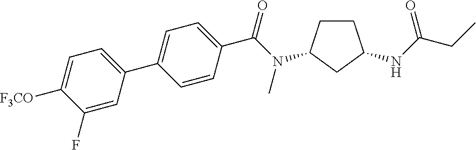 [SMILES: CCC(=O)N[C@H]1CC[C@@H](N(C)C(=O)c2ccc(-c3ccc(OC(F)(F)F)c(F)c3)cc2)C1] |
| 1536 | 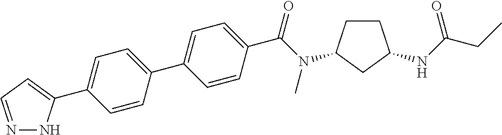 [SMILES: CCC(=O)N[C@H]1CC[C@@H](N(C)C(=O)c2ccc(-c3ccc(-c4ccn[nH]4)cc3)cc2)C1] |
| 1537 | 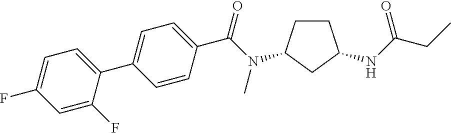 [SMILES: CCC(=O)N[C@H]1CC[C@@H](N(C)C(=O)c2ccc(-c3ccc(F)cc3F)cc2)C1] |
| 1538 | 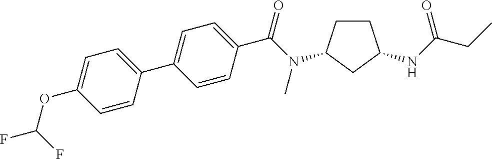 [SMILES: CCC(=O)N[C@H]1CC[C@@H](N(C)C(=O)c2ccc(-c3ccc(OC(F)F)cc3)cc2)C1] |
| 1539 | 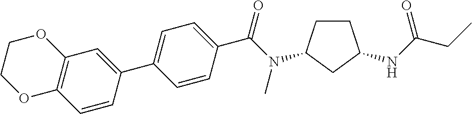 [SMILES: CCC(=O)N[C@H]1CC[C@@H](N(C)C(=O)c2ccc(-c3ccc4c(c3)OCCO4)cc2)C1] |
| 1540 | 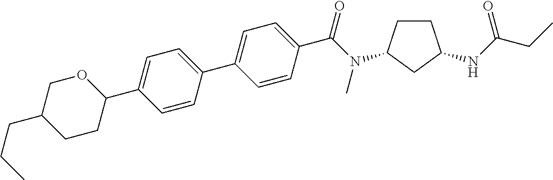 [SMILES: CCCC1CCC(c2ccc(-c3ccc(C(=O)N(C)[C@@H]4CC[C@H](NC(=O)CC)C4)cc3)cc2)OC1] |
| 1541 | 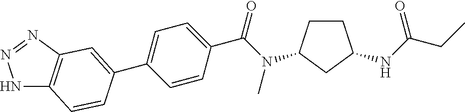 [SMILES: CCC(=O)N[C@H]1CC[C@@H](N(C)C(=O)c2ccc(-c3ccc4[nH]nnc4c3)cc2)C1] |
| 1542 | 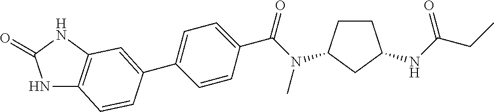 [SMILES: CCC(=O)N[C@H]1CC[C@@H](N(C)C(=O)c2ccc(-c3ccc4[nH]c(=O)[nH]c4c3)cc2)C1] |
| 1543 | 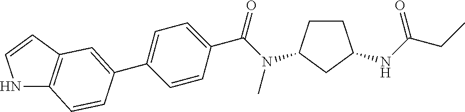 [SMILES: CCC(=O)N[C@H]1CC[C@@H](N(C)C(=O)c2ccc(-c3ccc4[nH]ccc4c3)cc2)C1] |
| 1544 | 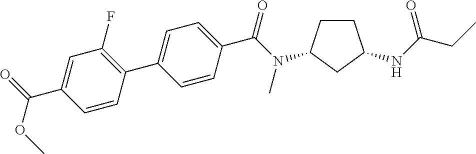 [SMILES: CCC(=O)N[C@H]1CC[C@@H](N(C)C(=O)c2ccc(-c3ccc(C(=O)OC)cc3F)cc2)C1] |
| 1545 | 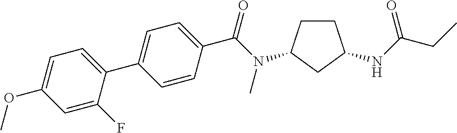 [SMILES: CCC(=O)N[C@H]1CC[C@@H](N(C)C(=O)c2ccc(-c3ccc(OC)cc3F)cc2)C1] |
| 1546 | 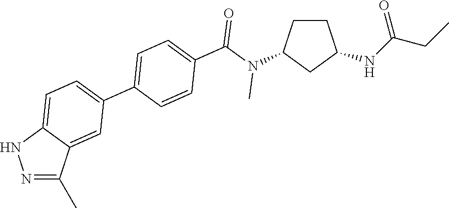 [SMILES: CCC(=O)N[C@H]1CC[C@@H](N(C)C(=O)c2ccc(-c3ccc4[nH]nc(C)c4c3)cc2)C1] |
| 1547 | 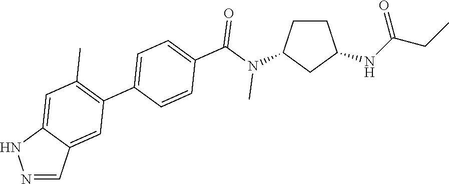 [SMILES: CCC(=O)N[C@H]1CC[C@@H](N(C)C(=O)c2ccc(-c3cc4cn[nH]c4cc3C)cc2)C1] |
| 1548 | 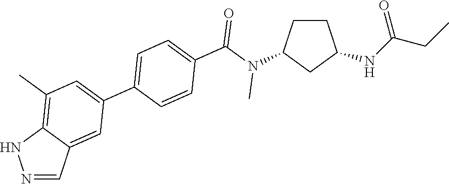 [SMILES: CCC(=O)N[C@H]1CC[C@@H](N(C)C(=O)c2ccc(-c3cc(C)c4[nH]ncc4c3)cc2)C1] |
| 1549 | 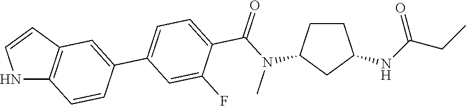 [SMILES: CCC(=O)N[C@H]1CC[C@@H](N(C)C(=O)c2ccc(-c3ccc4[nH]ccc4c3)cc2F)C1] |
| 1550 | 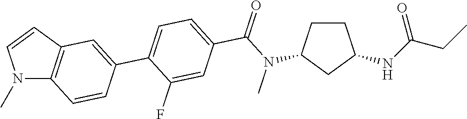 [SMILES: CCC(=O)N[C@H]1CC[C@@H](N(C)C(=O)c2ccc(-c3ccc4c(ccn4C)c3)c(F)c2)C1] |
| 1551 | 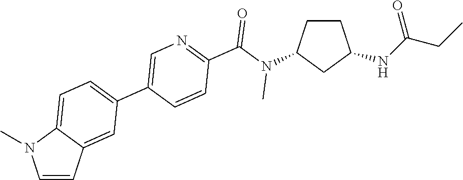 [SMILES: CCC(=O)N[C@H]1CC[C@@H](N(C)C(=O)c2ccc(-c3ccc4c(ccn4C)c3)cn2)C1] |
| 1552 | 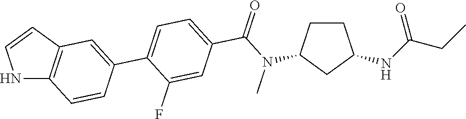 [SMILES: CCC(=O)N[C@H]1CC[C@@H](N(C)C(=O)c2ccc(-c3ccc4[nH]ccc4c3)c(F)c2)C1] |
| 1553 | 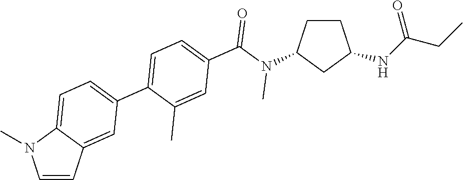 [SMILES: CCC(=O)N[C@H]1CC[C@@H](N(C)C(=O)c2ccc(-c3ccc4c(ccn4C)c3)c(C)c2)C1] |
| 1554 | 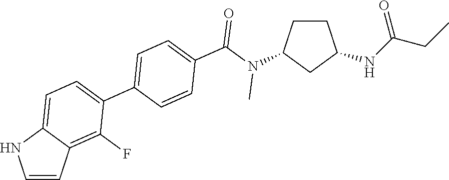 [SMILES: CCC(=O)N[C@H]1CC[C@@H](N(C)C(=O)c2ccc(-c3ccc4[nH]ccc4c3F)cc2)C1] |
| 1555 | 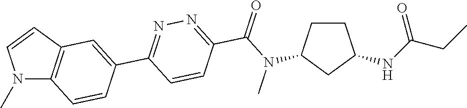 [SMILES: CCC(=O)N[C@H]1CC[C@@H](N(C)C(=O)c2ccc(-c3ccc4c(ccn4C)c3)nn2)C1] |
| 1556 | 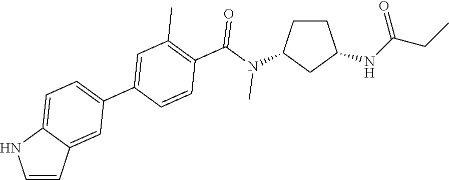 [SMILES: CCC(=O)N[C@H]1CC[C@@H](N(C)C(=O)c2ccc(-c3ccc4[nH]ccc4c3)cc2C)C1] |
| 1557 | 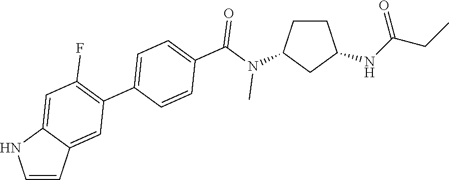 [SMILES: CCC(=O)N[C@H]1CC[C@@H](N(C)C(=O)c2ccc(-c3cc4cc[nH]c4cc3F)cc2)C1] |
| 1558 | 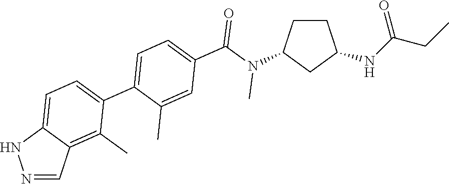 [SMILES: CCC(=O)N[C@H]1CC[C@@H](N(C)C(=O)c2ccc(-c3ccc4[nH]ncc4c3C)c(C)c2)C1] |
| 1559 | 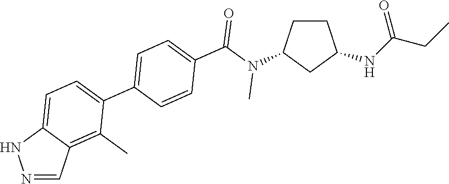 [SMILES: CCC(=O)N[C@H]1CC[C@@H](N(C)C(=O)c2ccc(-c3ccc4[nH]ncc4c3C)cc2)C1] |
| 1560 | 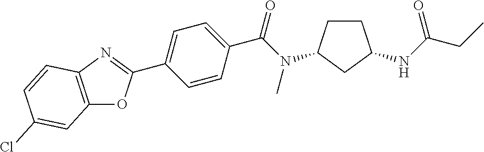 [SMILES: CCC(=O)N[C@H]1CC[C@@H](N(C)C(=O)c2ccc(-c3nc4ccc(Cl)cc4o3)cc2)C1] |
| 1561 | 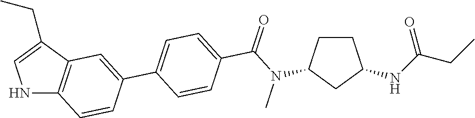 [SMILES: CCC(=O)N[C@H]1CC[C@@H](N(C)C(=O)c2ccc(-c3ccc4[nH]cc(CC)c4c3)cc2)C1] |
| 1562 | 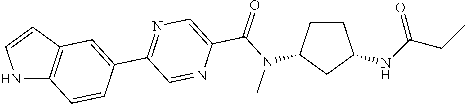 [SMILES: CCC(=O)N[C@H]1CC[C@@H](N(C)C(=O)c2cnc(-c3ccc4[nH]ccc4c3)cn2)C1] |
| 1563 | 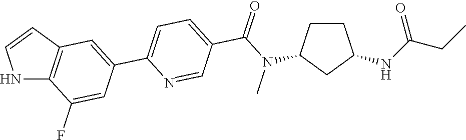 [SMILES: CCC(=O)N[C@H]1CC[C@@H](N(C)C(=O)c2ccc(-c3cc(F)c4[nH]ccc4c3)nc2)C1] |
| 1564 | 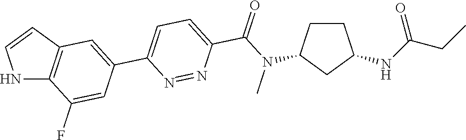 [SMILES: CCC(=O)N[C@H]1CC[C@@H](N(C)C(=O)c2ccc(-c3cc(F)c4[nH]ccc4c3)nn2)C1] |
| 1565 | 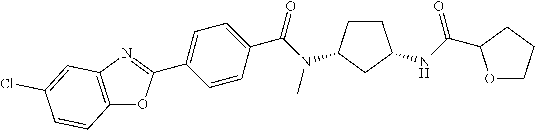 [SMILES: CN(C(=O)c1ccc(-c2nc3cc(Cl)ccc3o2)cc1)[C@@H]1CC[C@H](NC(=O)C2CCCO2)C1] |
| 1566 | 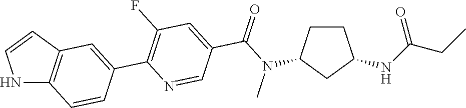 [SMILES: CCC(=O)N[C@H]1CC[C@@H](N(C)C(=O)c2cnc(-c3ccc4[nH]ccc4c3)c(F)c2)C1] |
| 1567 | 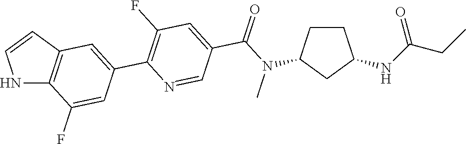 [SMILES: CCC(=O)N[C@H]1CC[C@@H](N(C)C(=O)c2cnc(-c3cc(F)c4[nH]ccc4c3)c(F)c2)C1] |
| 1568 | 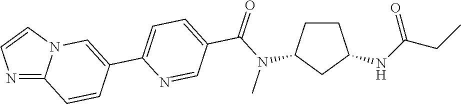 [SMILES: CCC(=O)N[C@H]1CC[C@@H](N(C)C(=O)c2ccc(-c3ccc4nccn4c3)nc2)C1] |
| 1569 | 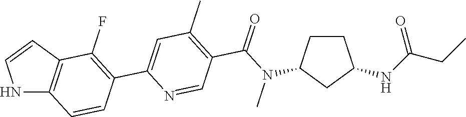 [SMILES: CCC(=O)N[C@H]1CC[C@@H](N(C)C(=O)c2cnc(-c3ccc4[nH]ccc4c3F)cc2C)C1] |
| 1570 | 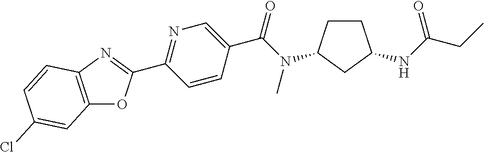 [SMILES: CCC(=O)N[C@H]1CC[C@@H](N(C)C(=O)c2ccc(-c3nc4ccc(Cl)cc4o3)nc2)C1] |
| 1571 | 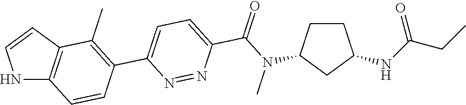 [SMILES: CCC(=O)N[C@H]1CC[C@@H](N(C)C(=O)c2ccc(-c3ccc4[nH]ccc4c3C)nn2)C1] |
| 1572 | 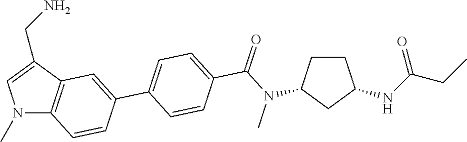 [SMILES: CCC(=O)N[C@H]1CC[C@@H](N(C)C(=O)c2ccc(-c3ccc4c(c3)c(CN)cn4C)cc2)C1] |
| 1573 | 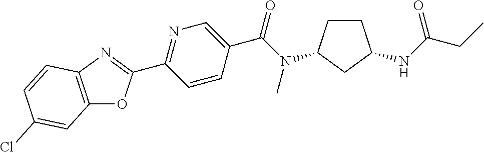 [SMILES: CCC(=O)N[C@H]1CC[C@@H](N(C)C(=O)c2ccc(-c3nc4ccc(Cl)cc4o3)nc2)C1] |
| 1574 | 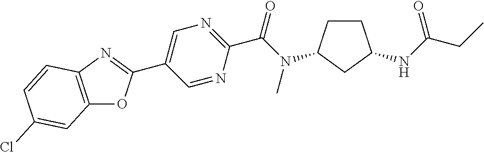 [SMILES: CCC(=O)N[C@H]1CC[C@@H](N(C)C(=O)c2ncc(-c3nc4ccc(Cl)cc4o3)cn2)C1] |
| 1575 | 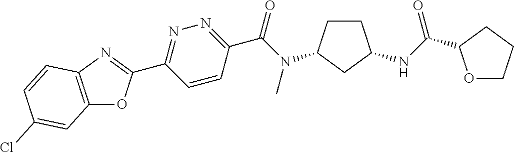 |
| 1576 | 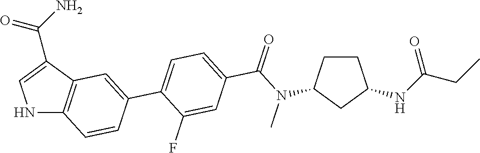 [SMILES: CCC(=O)N[C@H]1CC[C@@H](N(C)C(=O)c2ccc(-c3ccc4[nH]cc(C(N)=O)c4c3)c(F)c2)C1] |
| 1577 | 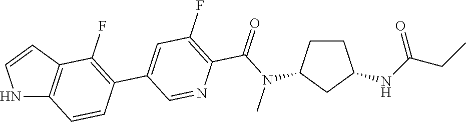 [SMILES: CCC(=O)N[C@H]1CC[C@@H](N(C)C(=O)c2ncc(-c3ccc4[nH]ccc4c3F)cc2F)C1] |
| 1578 | 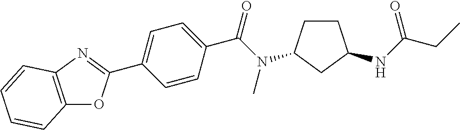 [SMILES: CCC(=O)N[C@@H]1CC[C@@H](N(C)C(=O)c2ccc(-c3nc4ccccc4o3)cc2)C1] |
| 1579 | 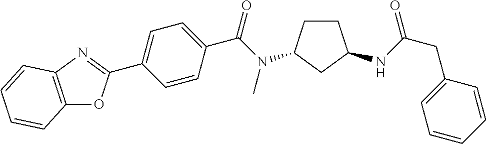 [SMILES: CN(C(=O)c1ccc(-c2nc3ccccc3o2)cc1)[C@@H]1CC[C@@H](NC(=O)Cc2ccccc2)C1] |
| 1580 | 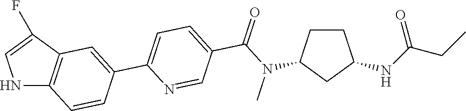 [SMILES: CCC(=O)N[C@H]1CC[C@@H](N(C)C(=O)c2ccc(-c3ccc4[nH]cc(F)c4c3)nc2)C1] |
| 1581 | 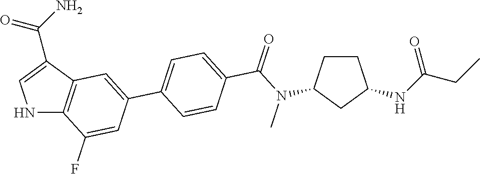 [SMILES: CCC(=O)N[C@H]1CC[C@@H](N(C)C(=O)c2ccc(-c3cc(F)c4[nH]cc(C(N)=O)c4c3)cc2)C1] |
| 1582 | 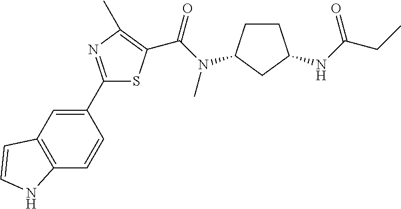 [SMILES: CCC(=O)N[C@H]1CC[C@@H](N(C)C(=O)c2sc(-c3ccc4[nH]ccc4c3)nc2C)C1] |
| 1583 | 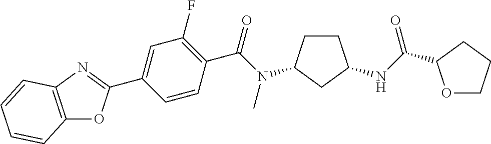 |
| 1584 | 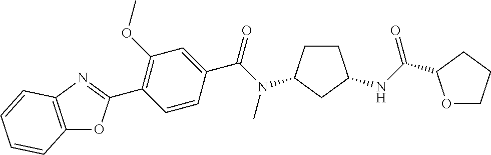 |
| 1585 | 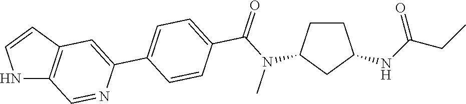 [SMILES: CCC(=O)N[C@H]1CC[C@@H](N(C)C(=O)c2ccc(-c3cc4cc[nH]c4cn3)cc2)C1] |
| 1586 | 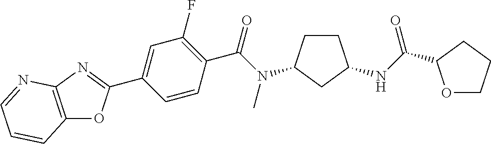 |
| 1587 | 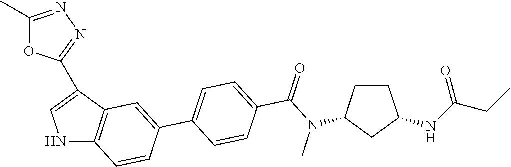 [SMILES: CCC(=O)N[C@H]1CC[C@@H](N(C)C(=O)c2ccc(-c3ccc4[nH]cc(-c5nnc(C)o5)c4c3)cc2)C1] |
| 1588 | 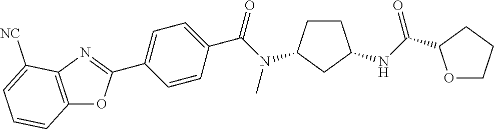 |
| 1589 | 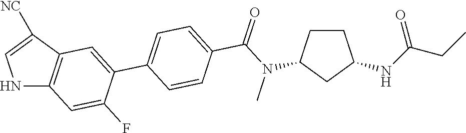 [SMILES: CCC(=O)N[C@H]1CC[C@@H](N(C)C(=O)c2ccc(-c3cc4c(C#N)c[nH]c4cc3F)cc2)C1] |
| 1590 | 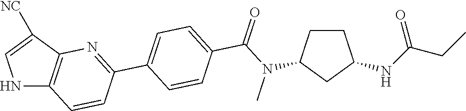 [SMILES: CCC(=O)N[C@H]1CC[C@@H](N(C)C(=O)c2ccc(-c3ccc4[nH]cc(C#N)c4n3)cc2)C1] |
| 1591 | 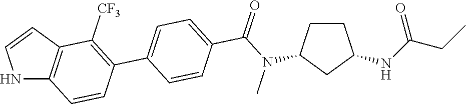 [SMILES: CCC(=O)N[C@H]1CC[C@@H](N(C)C(=O)c2ccc(-c3ccc4[nH]ccc4c3C(F)(F)F)cc2)C1] |
| 1592 | 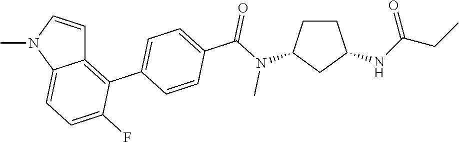 [SMILES: CCC(=O)N[C@H]1CC[C@@H](N(C)C(=O)c2ccc(-c3c(F)ccc4c3ccn4C)cc2)C1] |
| 1593 | 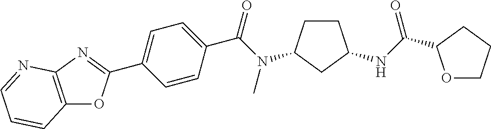 |
| 1594 | 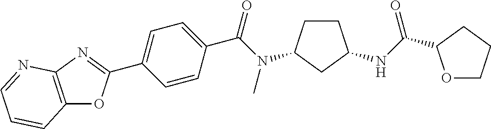 |
| 1595 | 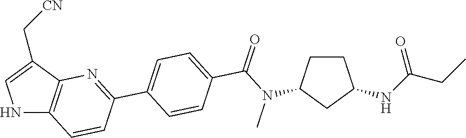 [SMILES: CCC(=O)N[C@H]1CC[C@@H](N(C)C(=O)c2ccc(-c3ccc4[nH]cc(CC#N)c4n3)cc2)C1] |
| 1596 | 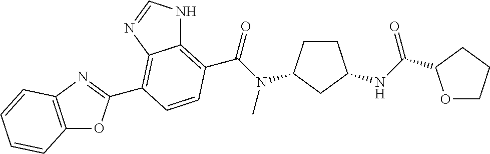 |
| 1597 | 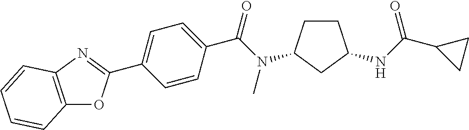 [SMILES: CN(C(=O)c1ccc(-c2nc3ccccc3o2)cc1)[C@@H]1CC[C@H](NC(=O)C2CC2)C1] |
| 1598 | 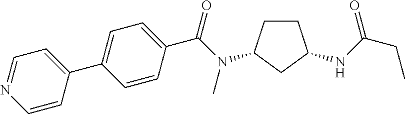 [SMILES: CCC(=O)N[C@H]1CC[C@@H](N(C)C(=O)c2ccc(-c3ccncc3)cc2)C1] |
| 1599 | 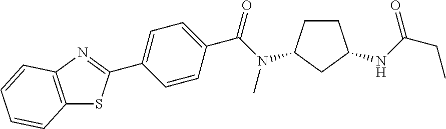 [SMILES: CCC(=O)N[C@H]1CC[C@@H](N(C)C(=O)c2ccc(-c3nc4ccccc4s3)cc2)C1] |
| 1600 | 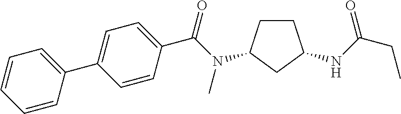 [SMILES: CCC(=O)N[C@H]1CC[C@@H](N(C)C(=O)c2ccc(-c3ccccc3)cc2)C1] |
| 1601 | 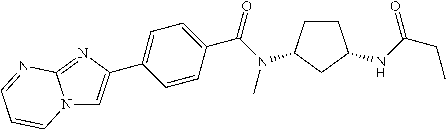 [SMILES: CCC(=O)N[C@H]1CC[C@@H](N(C)C(=O)c2ccc(-c3cn4cccnc4n3)cc2)C1] |
| 1602 | 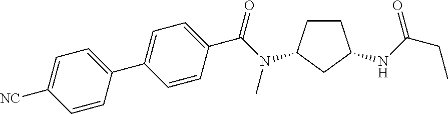 [SMILES: CCC(=O)N[C@H]1CC[C@@H](N(C)C(=O)c2ccc(-c3ccc(C#N)cc3)cc2)C1] |
| 1603 | 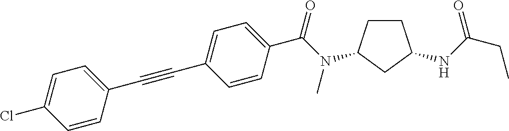 [SMILES: CCC(=O)N[C@H]1CC[C@@H](N(C)C(=O)c2ccc(C#Cc3ccc(Cl)cc3)cc2)C1] |
| 1604 | 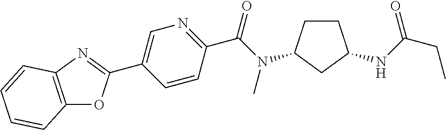 [SMILES: CCC(=O)N[C@H]1CC[C@@H](N(C)C(=O)c2ccc(-c3nc4ccccc4o3)cn2)C1] |
| 1605 | 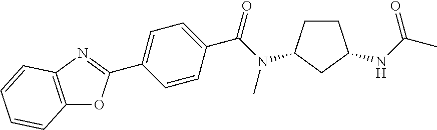 [SMILES: CC(=O)N[C@H]1CC[C@@H](N(C)C(=O)c2ccc(-c3nc4ccccc4o3)cc2)C1] |
| 1606 | 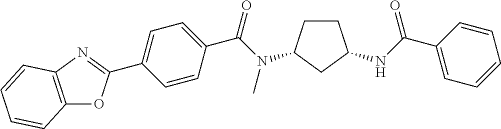 [SMILES: CN(C(=O)c1ccc(-c2nc3ccccc3o2)cc1)[C@@H]1CC[C@H](NC(=O)c2ccccc2)C1] |
| 1607 | 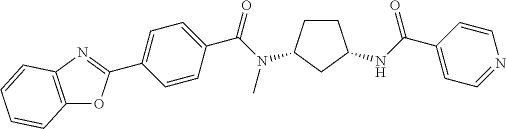 [SMILES: CN(C(=O)c1ccc(-c2nc3ccccc3o2)cc1)[C@@H]1CC[C@H](NC(=O)c2ccncc2)C1] |
| 1608 | 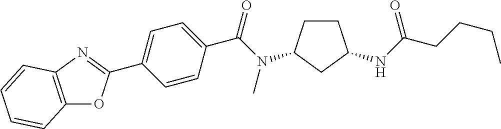 [SMILES: CCCCC(=O)N[C@H]1CC[C@@H](N(C)C(=O)c2ccc(-c3nc4ccccc4o3)cc2)C1] |
| 1609 | 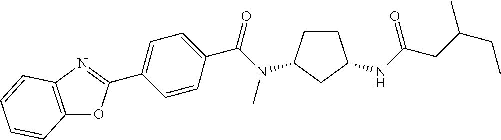 [SMILES: CCC(C)CC(=O)N[C@H]1CC[C@@H](N(C)C(=O)c2ccc(-c3nc4ccccc4o3)cc2)C1] |
| 1610 | 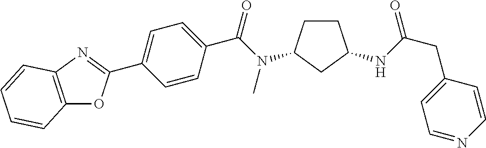 [SMILES: CN(C(=O)c1ccc(-c2nc3ccccc3o2)cc1)[C@@H]1CC[C@H](NC(=O)Cc2ccncc2)C1] |
| 1611 | 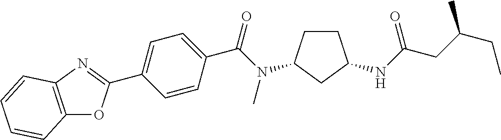 [SMILES: CC[C@H](C)CC(=O)N[C@H]1CC[C@@H](N(C)C(=O)c2ccc(-c3nc4ccccc4o3)cc2)C1] |
| 1612 | 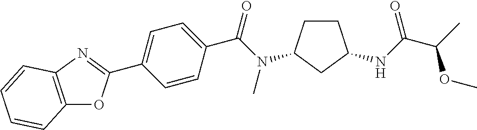 [SMILES: CO[C@H](C)C(=O)N[C@H]1CC[C@@H](N(C)C(=O)c2ccc(-c3nc4ccccc4o3)cc2)C1] |
| 1613 | 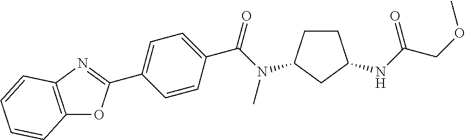 [SMILES: COCC(=O)N[C@H]1CC[C@@H](N(C)C(=O)c2ccc(-c3nc4ccccc4o3)cc2)C1] |
| 1614 | 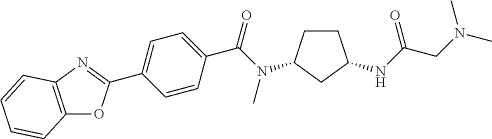 [SMILES: CN(C)CC(=O)N[C@H]1CC[C@@H](N(C)C(=O)c2ccc(-c3nc4ccccc4o3)cc2)C1] |
| 1615 | 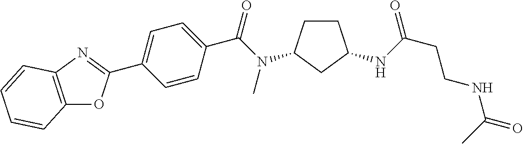 [SMILES: CC(=O)NCCC(=O)N[C@H]1CC[C@@H](N(C)C(=O)c2ccc(-c3nc4ccccc4o3)cc2)C1] |
| 1616 | 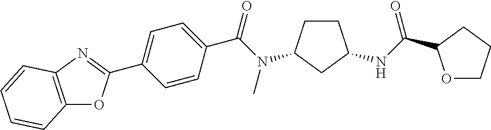 |
| 1617 | 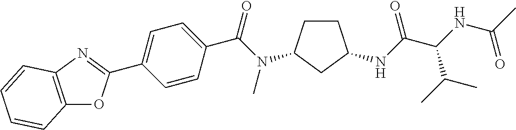 [SMILES: CC(=O)N[C@@H](C(=O)N[C@H]1CC[C@@H](N(C)C(=O)c2ccc(-c3nc4ccccc4o3)cc2)C1)C(C)C] |
| 1618 | 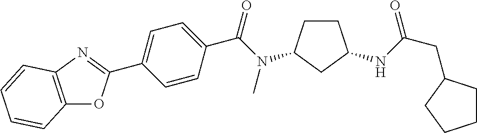 [SMILES: CN(C(=O)c1ccc(-c2nc3ccccc3o2)cc1)[C@@H]1CC[C@H](NC(=O)CC2CCCC2)C1] |
| 1619 | 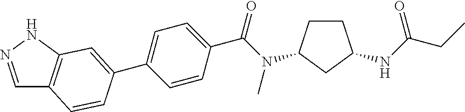 [SMILES: CCC(=O)N[C@H]1CC[C@@H](N(C)C(=O)c2ccc(-c3ccc4cn[nH]c4c3)cc2)C1] |
| 1620 | 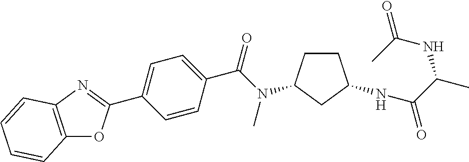 [SMILES: CC(=O)N[C@H](C)C(=O)N[C@H]1CC[C@@H](N(C)C(=O)c2ccc(-c3nc4ccccc4o3)cc2)C1] |
| 1621 | 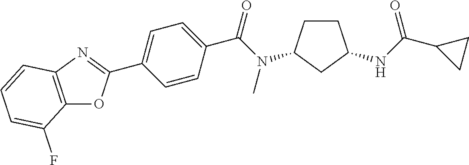 [SMILES: CN(C(=O)c1ccc(-c2nc3cccc(F)c3o2)cc1)[C@@H]1CC[C@H](NC(=O)C2CC2)C1] |
| 1622 | 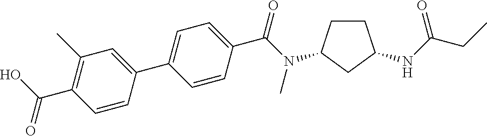 [SMILES: CCC(=O)N[C@H]1CC[C@@H](N(C)C(=O)c2ccc(-c3ccc(C(=O)O)c(C)c3)cc2)C1] |
| 1623 | 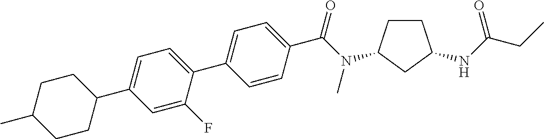 [SMILES: CCC(=O)N[C@H]1CC[C@@H](N(C)C(=O)c2ccc(-c3ccc(C4CCC(C)CC4)cc3F)cc2)C1] |
| 1624 | 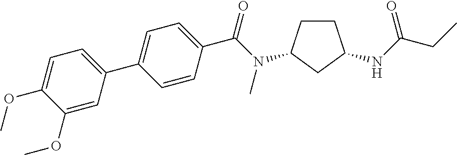 [SMILES: CCC(=O)N[C@H]1CC[C@@H](N(C)C(=O)c2ccc(-c3ccc(OC)c(OC)c3)cc2)C1] |
| 1625 | 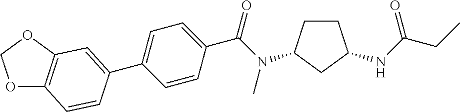 [SMILES: CCC(=O)N[C@H]1CC[C@@H](N(C)C(=O)c2ccc(-c3ccc4c(c3)OCO4)cc2)C1] |
| 1626 | 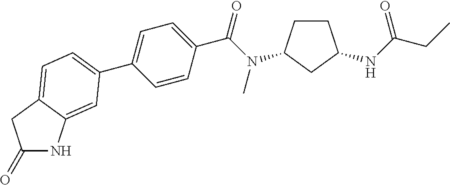 [SMILES: CCC(=O)N[C@H]1CC[C@@H](N(C)C(=O)c2ccc(-c3ccc4c(c3)NC(=O)C4)cc2)C1] |
| 1627 | 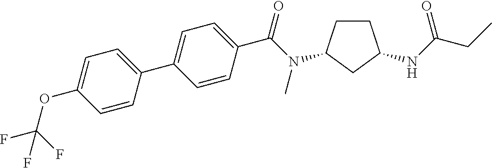 [SMILES: CCC(=O)N[C@H]1CC[C@@H](N(C)C(=O)c2ccc(-c3ccc(OC(F)(F)F)cc3)cc2)C1] |
| 1628 | 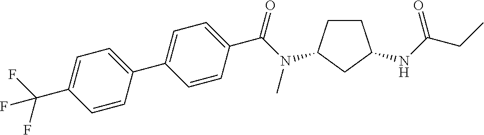 [SMILES: CCC(=O)N[C@H]1CC[C@@H](N(C)C(=O)c2ccc(-c3ccc(C(F)(F)F)cc3)cc2)C1] |
| 1629 | 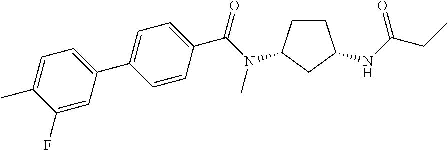 [SMILES: CCC(=O)N[C@H]1CC[C@@H](N(C)C(=O)c2ccc(-c3ccc(C)c(F)c3)cc2)C1] |
| 1630 | 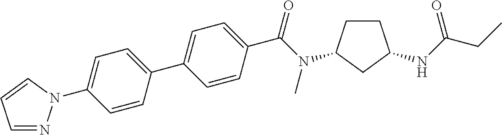 [SMILES: CCC(=O)N[C@H]1CC[C@@H](N(C)C(=O)c2ccc(-c3ccc(-n4cccn4)cc3)cc2)C1] |
| 1631 | 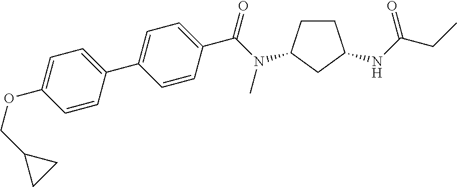 [SMILES: CCC(=O)N[C@H]1CC[C@@H](N(C)C(=O)c2ccc(-c3ccc(OCC4CC4)cc3)cc2)C1] |
| 1632 | 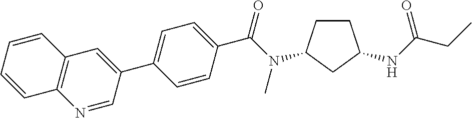 [SMILES: CCC(=O)N[C@H]1CC[C@@H](N(C)C(=O)c2ccc(-c3cnc4ccccc4c3)cc2)C1] |
| 1633 | 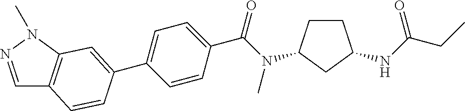 [SMILES: CCC(=O)N[C@H]1CC[C@@H](N(C)C(=O)c2ccc(-c3ccc4cnn(C)c4c3)cc2)C1] |
| 1634 | 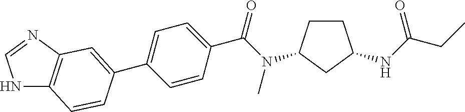 [SMILES: CCC(=O)N[C@H]1CC[C@@H](N(C)C(=O)c2ccc(-c3ccc4[nH]cnc4c3)cc2)C1] |
| 1635 | 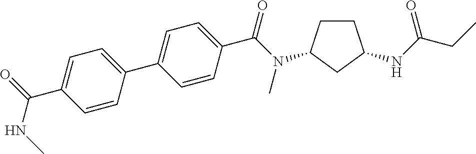 [SMILES: CCC(=O)N[C@H]1CC[C@@H](N(C)C(=O)c2ccc(-c3ccc(C(=O)NC)cc3)cc2)C1] |
| 1636 | 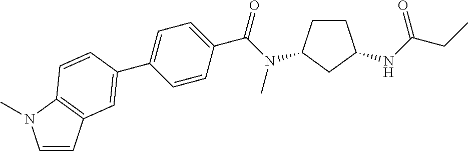 [SMILES: CCC(=O)N[C@H]1CC[C@@H](N(C)C(=O)c2ccc(-c3ccc4c(ccn4C)c3)cc2)C1] |
| 1637 | 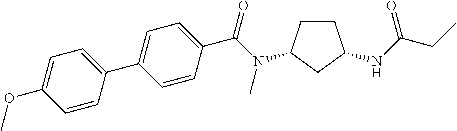 [SMILES: CCC(=O)N[C@H]1CC[C@@H](N(C)C(=O)c2ccc(-c3ccc(OC)cc3)cc2)C1] |
| 1638 | 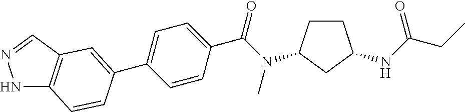 [SMILES: CCC(=O)N[C@H]1CC[C@@H](N(C)C(=O)c2ccc(-c3ccc4[nH]ncc4c3)cc2)C1] |
| 1639 | 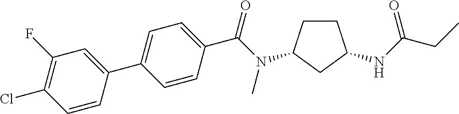 [SMILES: CCC(=O)N[C@H]1CC[C@@H](N(C)C(=O)c2ccc(-c3ccc(Cl)c(F)c3)cc2)C1] |
| 1640 | 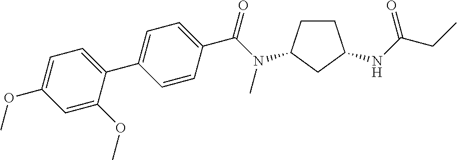 [SMILES: CCC(=O)N[C@H]1CC[C@@H](N(C)C(=O)c2ccc(-c3ccc(OC)cc3OC)cc2)C1] |
| 1641 | 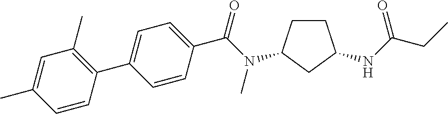 [SMILES: CCC(=O)N[C@H]1CC[C@@H](N(C)C(=O)c2ccc(-c3ccc(C)cc3C)cc2)C1] |
| 1642 | 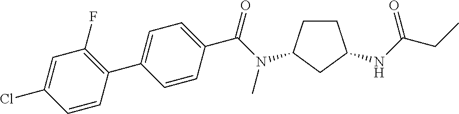 [SMILES: CCC(=O)N[C@H]1CC[C@@H](N(C)C(=O)c2ccc(-c3ccc(Cl)cc3F)cc2)C1] |
| 1643 | 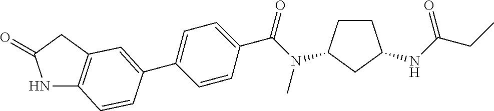 [SMILES: CCC(=O)N[C@H]1CC[C@@H](N(C)C(=O)c2ccc(-c3ccc4c(c3)CC(=O)N4)cc2)C1] |
| 1644 | 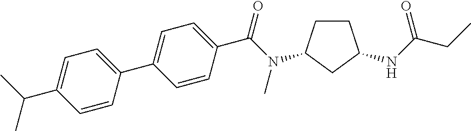 [SMILES: CCC(=O)N[C@H]1CC[C@@H](N(C)C(=O)c2ccc(-c3ccc(C(C)C)cc3)cc2)C1] |
| 1645 | 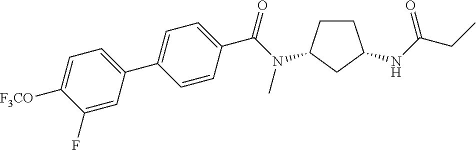 [SMILES: CCC(=O)N[C@H]1CC[C@@H](N(C)C(=O)c2ccc(-c3ccc(OC(F)(F)F)c(F)c3)cc2)C1] |
| 1646 | 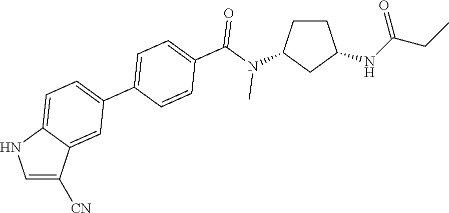 [SMILES: CCC(=O)N[C@H]1CC[C@@H](N(C)C(=O)c2ccc(-c3ccc4[nH]cc(C#N)c4c3)cc2)C1] |
| 1647 | 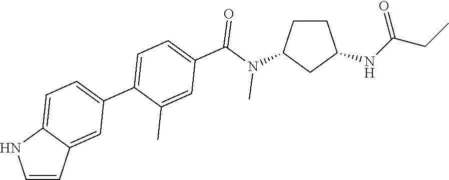 [SMILES: CCC(=O)N[C@H]1CC[C@@H](N(C)C(=O)c2ccc(-c3ccc4[nH]ccc4c3)c(C)c2)C1] |
| 1648 | 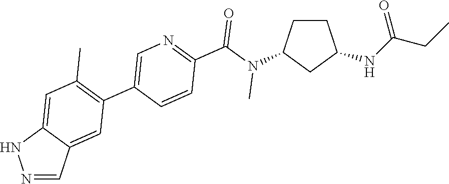 [SMILES: CCC(=O)N[C@H]1CC[C@@H](N(C)C(=O)c2ccc(-c3cc4cn[nH]c4cc3C)cn2)C1] |
| 1649 | 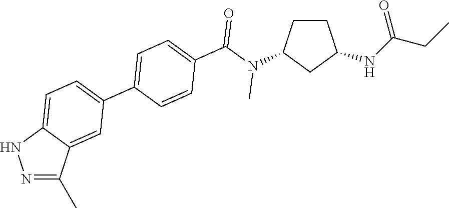 [SMILES: CCC(=O)N[C@H]1CC[C@@H](N(C)C(=O)c2ccc(-c3ccc4[nH]nc(C)c4c3)cc2)C1] |
| 1650 | 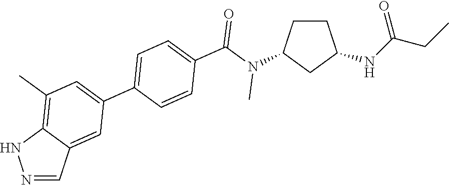 [SMILES: CCC(=O)N[C@H]1CC[C@@H](N(C)C(=O)c2ccc(-c3cc(C)c4[nH]ncc4c3)cc2)C1] |
| 1651 | 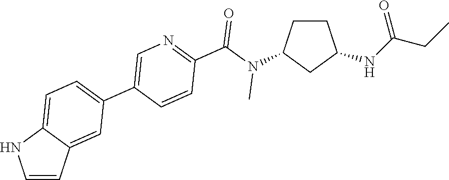 [SMILES: CCC(=O)N[C@H]1CC[C@@H](N(C)C(=O)c2ccc(-c3ccc4[nH]ccc4c3)cn2)C1] |
| 1652 | 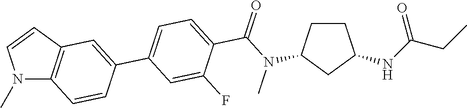 [SMILES: CCC(=O)N[C@H]1CC[C@@H](N(C)C(=O)c2ccc(-c3ccc4c(ccn4C)c3)cc2F)C1] |
| 1653 | 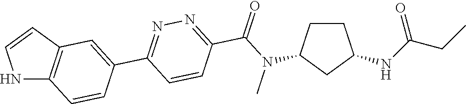 [SMILES: CCC(=O)N[C@H]1CC[C@@H](N(C)C(=O)c2ccc(-c3ccc4[nH]ccc4c3)nn2)C1] |
| 1654 | 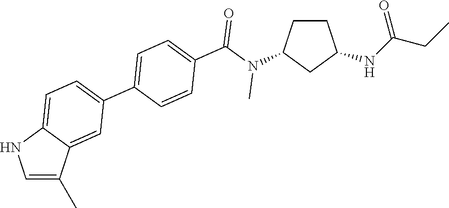 [SMILES: CCC(=O)N[C@H]1CC[C@@H](N(C)C(=O)c2ccc(-c3ccc4[nH]cc(C)c4c3)cc2)C1] |
| 1655 | 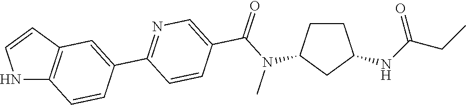 [SMILES: CCC(=O)N[C@H]1CC[C@@H](N(C)C(=O)c2ccc(-c3ccc4[nH]ccc4c3)nc2)C1] |
| 1656 | 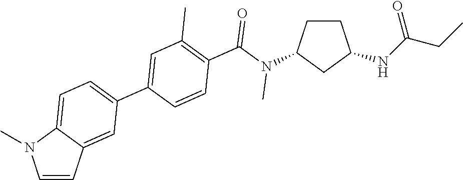 [SMILES: CCC(=O)N[C@H]1CC[C@@H](N(C)C(=O)c2ccc(-c3ccc4c(ccn4C)c3)cc2C)C1] |
| 1657 | 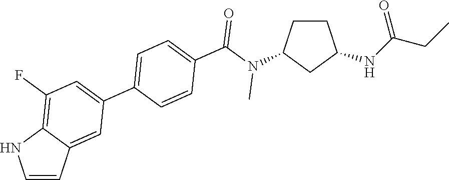 [SMILES: CCC(=O)N[C@H]1CC[C@@H](N(C)C(=O)c2ccc(-c3cc(F)c4[nH]ccc4c3)cc2)C1] |
| 1658 | 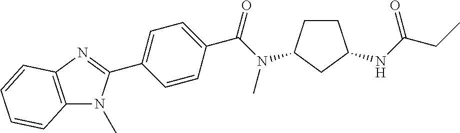 [SMILES: CCC(=O)N[C@H]1CC[C@@H](N(C)C(=O)c2ccc(-c3nc4ccccc4n3C)cc2)C1] |
| 1659 | 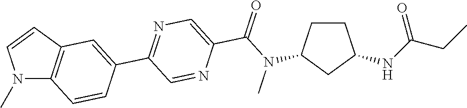 [SMILES: CCC(=O)N[C@H]1CC[C@@H](N(C)C(=O)c2cnc(-c3ccc4c(ccn4C)c3)cn2)C1] |
| 1660 | 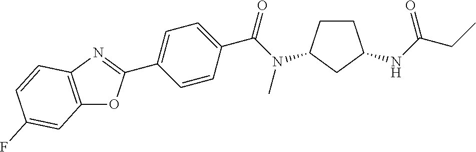 [SMILES: CCC(=O)N[C@H]1CC[C@@H](N(C)C(=O)c2ccc(-c3nc4ccc(F)cc4o3)cc2)C1] |
| 1661 | 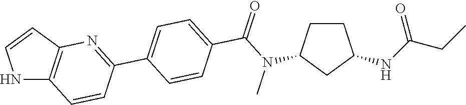 [SMILES: CCC(=O)N[C@H]1CC[C@@H](N(C)C(=O)c2ccc(-c3ccc4[nH]ccc4n3)cc2)C1] |
| 1662 | 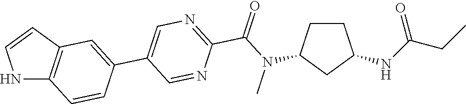 [SMILES: CCC(=O)N[C@H]1CC[C@@H](N(C)C(=O)c2ncc(-c3ccc4[nH]ccc4c3)cn2)C1] |
| 1663 | 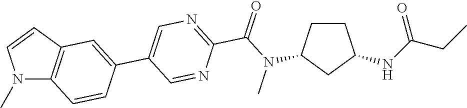 [SMILES: CCC(=O)N[C@H]1CC[C@@H](N(C)C(=O)c2ncc(-c3ccc4c(ccn4C)c3)cn2)C1] |
| 1664 | 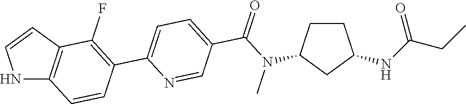 [SMILES: CCC(=O)N[C@H]1CC[C@@H](N(C)C(=O)c2ccc(-c3ccc4[nH]ccc4c3F)nc2)C1] |
| 1665 | 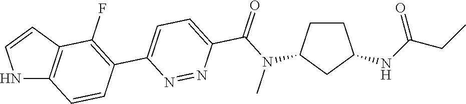 [SMILES: CCC(=O)N[C@H]1CC[C@@H](N(C)C(=O)c2ccc(-c3ccc4[nH]ccc4c3F)nn2)C1] |
| 1666 | 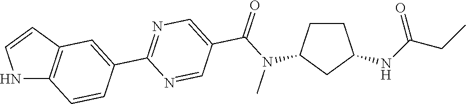 [SMILES: CCC(=O)N[C@H]1CC[C@@H](N(C)C(=O)c2cnc(-c3ccc4[nH]ccc4c3)nc2)C1] |
| 1667 | 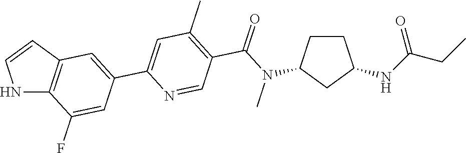 [SMILES: CCC(=O)N[C@H]1CC[C@@H](N(C)C(=O)c2cnc(-c3cc(F)c4[nH]ccc4c3)cc2C)C1] |
| 1668 | 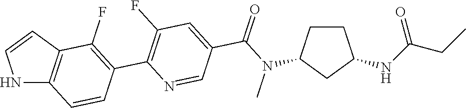 [SMILES: CCC(=O)N[C@H]1CC[C@@H](N(C)C(=O)c2cnc(-c3ccc4[nH]ccc4c3F)c(F)c2)C1] |
| 1669 | 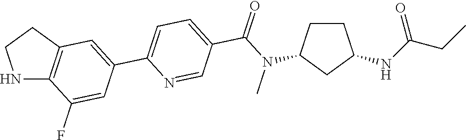 [SMILES: CCC(=O)N[C@H]1CC[C@@H](N(C)C(=O)c2ccc(-c3cc(F)c4c(c3)CCN4)nc2)C1] |
| 1670 | 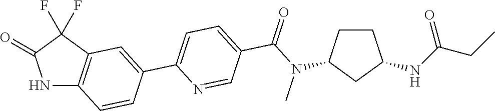 [SMILES: CCC(=O)N[C@H]1CC[C@@H](N(C)C(=O)c2ccc(-c3ccc4c(c3)C(F)(F)C(=O)N4)nc2)C1] |
| 1671 | 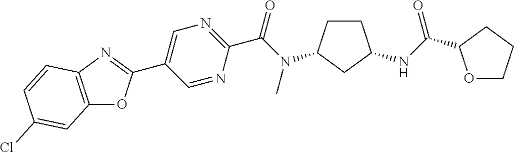 |
| 1672 | 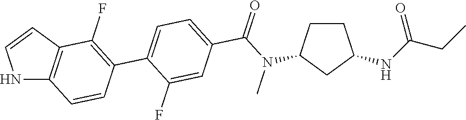 [SMILES: CCC(=O)N[C@H]1CC[C@@H](N(C)C(=O)c2ccc(-c3ccc4[nH]ccc4c3F)c(F)c2)C1] |
| 1673 | 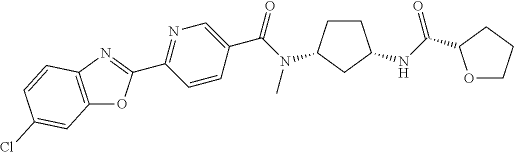 |
| 1674 | 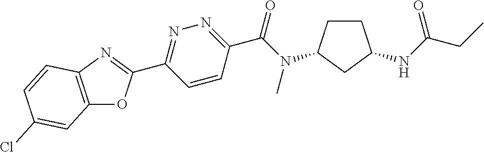 [SMILES: CCC(=O)N[C@H]1CC[C@@H](N(C)C(=O)c2ccc(-c3nc4ccc(Cl)cc4o3)nn2)C1] |
| 1675 | 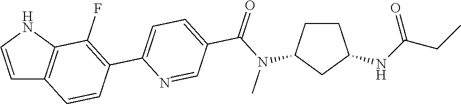 [SMILES: CCC(=O)N[C@H]1CC[C@@H](N(C)C(=O)c2ccc(-c3ccc4cc[nH]c4c3F)nc2)C1] |
| 1676 | 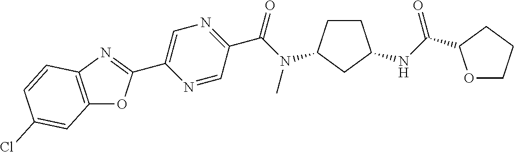 |
| 1677 | 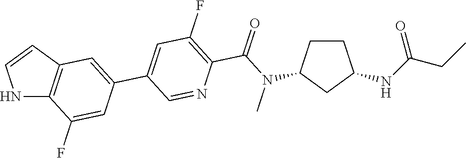 [SMILES: CCC(=O)N[C@H]1CC[C@@H](N(C)C(=O)c2ncc(-c3cc(F)c4[nH]ccc4c3)cc2F)C1] |
| 1678 | 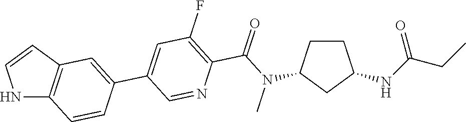 [SMILES: CCC(=O)N[C@H]1CC[C@@H](N(C)C(=O)c2ncc(-c3ccc4[nH]ccc4c3)cc2F)C1] |
| 1679 | 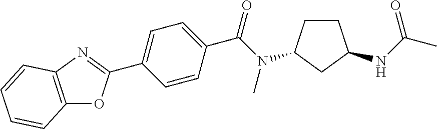 [SMILES: CC(=O)N[C@@H]1CC[C@@H](N(C)C(=O)c2ccc(-c3nc4ccccc4o3)cc2)C1] |
| 1680 | 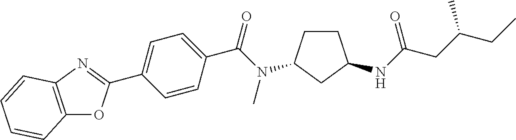 [SMILES: CC[C@@H](C)CC(=O)N[C@@H]1CC[C@@H](N(C)C(=O)c2ccc(-c3nc4ccccc4o3)cc2)C1] |
| 1681 | 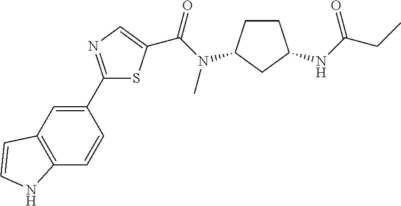 [SMILES: CCC(=O)N[C@H]1CC[C@@H](N(C)C(=O)c2cnc(-c3ccc4[nH]ccc4c3)s2)C1] |
| 1682 | 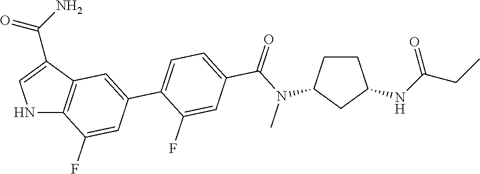 [SMILES: CCC(=O)N[C@H]1CC[C@@H](N(C)C(=O)c2ccc(-c3cc(F)c4[nH]cc(C(N)=O)c4c3)c(F)c2)C1] |
| 1683 | 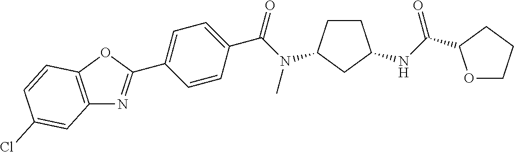 |
| 1684 | 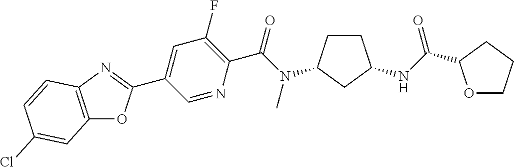 |
| 1685 | 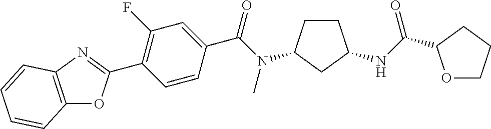 |
| 1686 | 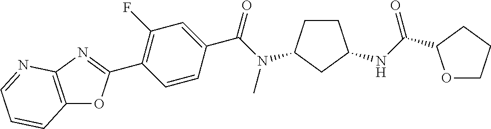 |
| 1687 | 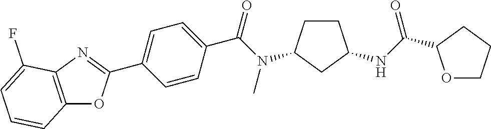 |
| 1688 | 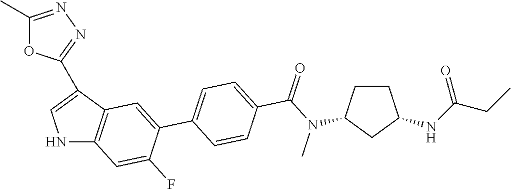 [SMILES: CCC(=O)N[C@H]1CC[C@@H](N(C)C(=O)c2ccc(-c3cc4c(-c5nnc(C)o5)c[nH]c4cc3F)cc2)C1] |
| 1689 | 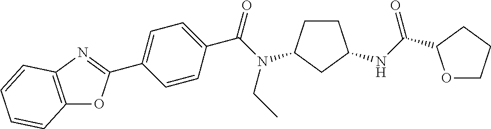 |
| 1690 | 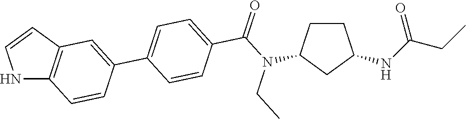 [SMILES: CCC(=O)N[C@H]1CC[C@@H](N(CC)C(=O)c2ccc(-c3ccc4[nH]ccc4c3)cc2)C1] |
| 1691 | 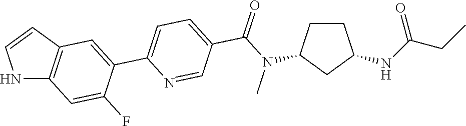 [SMILES: CCC(=O)N[C@H]1CC[C@@H](N(C)C(=O)c2ccc(-c3cc4cc[nH]c4cc3F)nc2)C1] |
| 1692 | 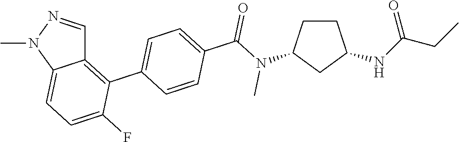 [SMILES: CCC(=O)N[C@H]1CC[C@@H](N(C)C(=O)c2ccc(-c3c(F)ccc4c3cnn4C)cc2)C1] |
| 1693 | 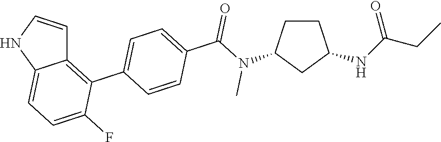 [SMILES: CCC(=O)N[C@H]1CC[C@@H](N(C)C(=O)c2ccc(-c3c(F)ccc4[nH]ccc34)cc2)C1] |
| 1694 | 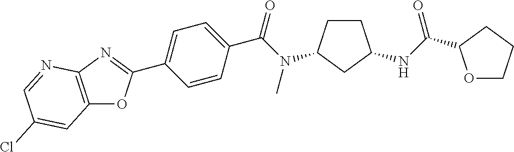 |
| 1695 | 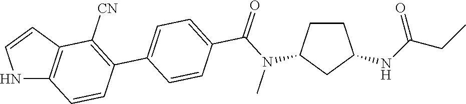 [SMILES: CCC(=O)N[C@H]1CC[C@@H](N(C)C(=O)c2ccc(-c3ccc4[nH]ccc4c3C#N)cc2)C1] |
| 1696 | 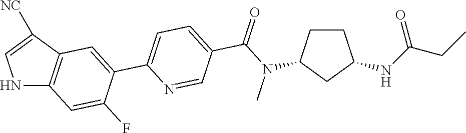 [SMILES: CCC(=O)N[C@H]1CC[C@@H](N(C)C(=O)c2ccc(-c3cc4c(C#N)c[nH]c4cc3F)nc2)C1] |

In some embodiments, the compound has the structure of Formula (XXXV):

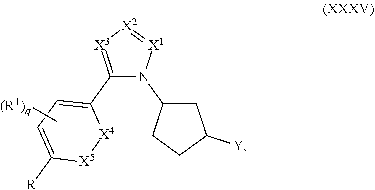

Wherein R is Ar or Het, Y is —CO—W or —NR⁴CO—W¹, W is NR²R², Het¹, CH₂Het¹, A, Cyc, Ar or CH₂Ar, —CONR²R²′ or Het¹, W¹ is NR²R², Het¹, CH₂Het¹, A, Cyc, Ar, CH₂Ar, CH₂Cyc or CH(OH)CH₂OH, R¹ is H, F, Cl, Br, OH, CN, NO₂, A, OA, SA′, SO₂Me, COA, CONH₂, CONHA′ or CONA′₂, R², R² each, independently of one another, denote H, A or [C(R³)₂]ₙCyc, each X¹, X², X³, is, independently, CR⁸ or N, X⁴ is CR⁸ or N, X⁵ is CR⁸ or N, R⁴ is H or A′, A is unbranched or branched alkyl with 1-10 C-atoms, wherein two adjacent carbon atoms may form a double bond and/or one or two non-adjacent CH— and/or CH₂— groups may be replaced by N-, O- and/or S-atoms and wherein 1-7H-atoms may be replaced by R⁵, Cyc is cycloalkyl with 3-7 C-atoms, which is unsubstituted or monosubstituted by OH, Hal or A, A′ is unbranched or branched alkyl with 1-6 C-atoms, wherein 1-5H-atoms may be replaced by F, R⁵ is F, C₁ or OH, Ar is phenyl, which is unsubstituted or mono-, di-, tri-, tetra- or pentasubstituted by Hal, A, O[C(R³)₂]ₙHet¹, Ar¹, [C(R³)₂]ₚOR³, [C(R³)₂]ₚNR³₂, NO₂, CN, [C(R³)₂]ₚCOOR³, CONR³₂, Het¹, OCH₂Cyc, [C(R³)₂]ₚNR³₂, NR³₂COA, NR³SO₂A, [C(R³)₂]ₚSO₂NR³₂, S(O)ₙA, O[C(R³)₂]ₘNR³₂, NHCOOA, NHCONR³₂ and/or COA, Ar¹ is phenyl or naphthyl, which is unsubstituted or mono-, di-, tri-, tetra- or pentasubstituted by Hal, A, [C(R³)₂]ₚOR³, [C(R³)₂]ₚNR³₂, NO₂, CN, [C(R³)₂]ₚCOOR³, [C(R³)₂]ₚNR³₂, NR³₂COA, NR³SO₂A, [C(R³)₂]ₚSO₂NR³₂, S(O)ₙA, O[C(R³)₂]ₘNR³₂, NHCOOA, NHCONR³₂ and/or COA, R³ is H or unbranched or branched alkyl with 1-6 C-atoms, R⁸ is H or A′, Het is a mono- or bicyclic saturated, unsaturated or aromatic heterocycle having 1 to 4 N, O and/or S atoms, which is unsubstituted or mono-, di-, tri-, tetra- or pentasubstituted by Hal, A, [C(R³)₂]ₙOA′, [C(R³)₂]ₙNR³₂, SR³, NO₂, CN, COOR³, CONR³₂, COHet¹, NR³COA, NR³SO₂A, SO₂NR³₂, S(O)ₙA, O[C(R³)₂]ₘNR³₂, NHCOOA, NHCONR³₂, CHO, COA, ═S, ═NH, ═NA and/or ═O (carbonyl oxygen), Het¹ is a mono- or bicyclic saturated, unsaturated or aromatic heterocycle having 1 to 4 N, O and/or S atoms, which is unsubstituted or mono-, di-, tri-, tetra- or pentasubstituted by Hal, A, [C(R³)₂]ₙOR³, [C(R³)₂]ₙNR³₂, SR³, NO₂, CN, COOR³, CONR³₂, NR³COA, NR³SO₂A, SO₂NR³₂, S(O)ₙA, O[C(R³)₂]ₘNR³₂, NHCOOA, NHCONR³₂) CHO, COA, =S═NH, ═NA and/or ═O (carbonyl oxygen), Hal is F, Cl, Br or I, m is 1, 2 or 3, n is 0, 1 or 2, p is 0, 1, 2, 3 or 4, q is 0, 1, 2 or 3, and pharmaceutically acceptable salts, tautomers and stereoisomers thereof, including mixtures thereof in all ratios.

In some embodiments, the compound is cis-configured and has the structure of Formula (XXXV-A):

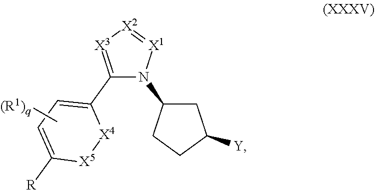

wherein the cyclopentane is 1,3-cis-disubstituted.

Preferably only one or two of X¹, X², X³ denote N. Furthermore, preferably X⁴ and X⁵ denote CR⁸.

In some embodiments, A is alkyl, this is unbranched (linear) or branched, and has 1, 2, 3, 4, 5, 6, 7, 8, 9 or 10 C atoms. A preferably is methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methyl-propyl, 1,1,2- or 1,2,2-trimethylpropyl, furthermore preferably, for example, trifluoromethyl. In further embodiments, A preferably is unbranched or branched alkyl with 1-10 C-atoms, wherein one or two non-adjacent CH— and/or CH₂— groups may be replaced by N- and/or O-atoms and wherein 1-7H-atoms may be replaced by R⁵ wherein 1-7H-atoms may be replaced by R⁵. In further embodiments, A very particularly preferably is alkyl having 1, 2, 3, 4, 5 or 6 C atoms, preferably methyl, ethyl, propyl, isopropyl, butyl, isobutyl, sec-butyl, tert-butyl, pentyl, hexyl, trifluoromethyl, pentafluoroethyl or 1,1,1-trifluoroethyl. In some embodiments, A is CH₂OCH₃, CH₂CH₂OH or CH₂CH₂OCH₃. In some embodiments, Cyc is cyclopropyl, cyclobutyl, cyclopentyl, cyclohexyl or cycloheptyl, preferably unsubstituted or monosubstituted by A. In some embodiments, A′ is alkyl, this is unbranched (linear) or branched, and has 1, 2, 3, 4, 5 or 6 C atoms. A′ preferably is methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, furthermore preferably, for example, trifluoromethyl. In some embodiments, A′ very particularly preferably is alkyl having 1, 2, 3, 4, 5 or 6 C atoms. In some embodiments, R¹ preferably is H or F. In some embodiments, R² preferably is H. In some embodiments, R^{2′} preferably is A or [C(R³)₂]ₙCyc. In some embodiments, R³ preferably is H, methyl, ethyl, propyl, isopropyl, butyl, pentyl or hexyl, particularly preferably H or methyl. In some embodiments, R⁴ preferably is H. In some embodiments, R⁵ preferably is F or Cl. In some embodiments, R⁸ preferably is H, methyl, ethyl, propyl or butyl, particularly preferably H or methyl.

In some embodiments, Ar is preferably o-, m- or p-tolyl, o-, m- or p-ethylphenyl, o-, m- or p-propylphenyl, o-, m- or p-isopropylphenyl, o-, m- or p-tert-butylphenyl, o-, m- or p-hydroxyphenyl, o-, m- or p-nitrophenyl, o-, m- or p-aminophenyl, o-, m- or p-(N-methylamino)phenyl, o-, m- or p-(N-methylaminocarbonyl)phenyl, o-, m- or p-methoxyphenyl, o-, m- or p-ethoxyphenyl, o-, m- or p-ethoxycarbonyl-phenyl, o-, m- or p-(N,N-dimethylamino)phenyl, o-, m- or p-(N,N-dimethyl-aminocarbonyl)phenyl, o-, m- or p-(N-ethylamino)phenyl, o-, m- or p-(N,N-diethylamino)phenyl, o-, m- or p-fluorophenyl, o-, m- or p-bromophenyl, o-, m- or p-chlorophenyl, o-, m- or p-(methylsulfonamido)phenyl, o-, m- or p-(methyl-sulfonyl)phenyl, o-, m- or p-cyanophenyl, o-, m- or p-carboxyphenyl, o-, m- or p-methoxycarbonylphenyl, o-, m- or p-acetylphenyl, o-, m- or p-amino-sulfonylphenyl, o-, m- or p-[2-(morpholin-4-yl)ethoxy]phenyl, o-, m- or p-[3-(N,N-diethylamino)propoxy]phenyl, furthermore preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,4- or 2,5-dinitrophenyl, 2,5- or 3,4-dimethoxyphenyl, 3-nitro-4-chlorophenyl, 3-amino-4-chloro-, 2-amino-3-chloro-, 2-amino-4-chloro-, 2-amino-5-chloro- or 2-amino-6-chlorophenyl, 2-nitro-4-N,N-dimethylamino- or 3-nitro-4-N,N-dimethylaminophenyl, 2,3-diaminophenyl, 2,3,4-, 2,3,5-, 2,3,6-, 2,4,6- or 3,4,5-trichlorophenyl, 2,4,6-trimethoxyphenyl, 2-hydroxy-3,5-dichlorophenyl, p-iodophenyl, 3,6-dichloro-4-aminophenyl, 4-fluoro-3-chlorophenyl, 2-fluoro-4-bromophenyl, 2,5-difluoro-4-bromophenyl, 3-bromo-6-methoxyphenyl, 3-chloro-6-methoxyphenyl, 3-chloro-4-acetamidophenyl, 3-fluoro-4-methoxyphenyl, 3-amino-6-methylphenyl, 3-chloro-4-acetamidophenyl or 2,5-dimethyl-4-chlorophenyl. In some embodiments, Ar furthermore preferably is phenyl, which is unsubstituted or mono-, di- or trisubstituted by Hal, A, Het¹, [C(R³)₂]ₚOR³, [C(R³)₂]ₚCOOR³, OCH₂Cyc, CONR³₂ and/or CN. In some embodiments, Ar¹ preferably is phenyl or naphthyl.

In some embodiments, Het is, for example, 2- or 3-furyl, 2- or 3-thienyl, 1-, 2- or 3-pyrrolyl, 1-, 2, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazoM-, -4- or -5-yl, 1,2,4-triazol-1-, -3- or 5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, indazolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-iso-quinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, further preferably 1,3-benzodioxol-5-yl, 1,4-benzodioxan-6-yl, 2,1,3-benzothiadiazol-4-, -5-yl or 2,1,3-benzoxadiazol-5-yl, azabicyclo[3.2.1]octyl or dibenzofuranyl. The heterocyclic radicals may also be partially or fully hydrogenated. In some embodiments, Het can thus also denote, for example, 2,3-dihydro-2-, -3-, -4- or -5-furyl, 2,5-dihydro-2-, -3-, -4- or 5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-,-3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-, -2-, -3-, -4-, -5-, -6-, -7- or -8-isoquinolyl, 2-, 3-, 5-, 6-, 7- or 8-3,4-dihydro-2H-benzo-1,4-oxazinyl, furthermore preferably 2,3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydrobenzofuran-5- or 6-yl, 2,3-(2-oxomethylenedioxy)phenyl or also 3,4-dihydro-2H-1,5-benzodioxepin-6- or -7-yl, furthermore preferably 2,3-dihydrobenzofuranyl, 2,3-dihydro-2-oxofuranyl, 3,4-dihydro-2-oxo-1/-/-quinazolinyl, 2,3-dihydrobenzoxazolyl, 2-oxo-2,3-di-hydrobenzoxazolyl, 2,3-dihydrobenzimidazolyl, 1,3-dihydroindole, 2-oxo-1,3-dihydroindole or 2-oxo-2,3-dihydrobenzimidazolyl. In some embodiments, Het preferably is a mono- or bicyclic aromatic heterocycle having 1 to 4 N, O and/or S atoms, which is unsubstituted or mono- or disubstituted by Hal. Het furthermore preferably is furyl, thienyl, pyrrolyl, imidazolyl, pyrazolyl, oxazolyl, isoxazolyl, thiazolyl, isothiazolyl, pyridyl, pyrimidinyl, triazolyl, tetrazolyl, oxadiazolyl, thiadiazolyl, pyridazinyl, pyrazinyl, benzoxazolyl, benzothiazolyl, benzimidazolyl, benzotriazolyl, indolyl, benzo-1,3-dioxolyl, benzodioxanyl, benzothiadiazolyl, indazolyl, benzofuranyl, quinolyl, isoquinolyl, pyrrolo[2,3-b]pyridinyl, oxazolo[5,4-b]pyridyl, imidazo[1,2-a]pyrimidinyl or oxazolo[5,4-c]pyridyl, each of which is unsubstituted or mono- or disubstituted by Hal. In some embodiments, Het furthermore preferably is furyl, thienyl, pyrrolyl, imidazolyl, pyrazolyl, oxazolyl, isoxazolyl, thiazolyl, isothiazolyl, pyridyl, pyrimidinyl, pyrrolo[2,3-b]pyridinyl, imidazo[1,2-a]pyrimidinyl, benzoxazolyl, benzothiazolyl or benzimidazolyl, each of which is unsubstituted or mono- or disubstituted by Hal. In some embodiments, Het furthermore preferably is furyl, thienyl, pyrrolyl, imidazolyl, pyrazolyl, oxazolyl, isoxazolyl, thiazolyl, isothiazolyl, pyridyl, pyrimidinyl, triazolyl, tetrazolyl, oxadiazolyl, thiadiazolyl, pyridazinyl, pyrazinyl, benzoxazolyl, benzothiazolyl, benzimidazolyl, benzotriazolyl, indolyl, benzo-1,3-dioxolyl, benzodioxanyl, benzothiadiazolyl, indazolyl, benzofuranyl, quinolyl, isoquinolyl, pyrrolo[2,3-b]pyridyl, oxazolo[5,4-b]pyridyl, imidazo[1,2-a]pyrimidinyl, 2,3-dihydro-indolyl, 2,3-dihydro-benzimidazolyl, imidazo[1,2-a]pyridyl, pyrrolo[3,2-b]pyridyl or oxazolo[5,4-c]pyridyl, each of which is unsubstituted or mono- or disubstituted by Hal, A and/or ═O. In some embodiments, Het furthermore preferably is a mono- or bicyclic aromatic heterocycle having 1 to 4 N, O and/or S atoms, which is unsubstituted or mono- or disubstituted by Hal, A and/or ═O. In some embodiments, Het¹ is, for example, 2- or 3-furyl, 2- or 3-thienyl, 1-, 2- or 3-pyrrolyl, 1-, 2, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-, -3- or 5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, indazolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-iso-quinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, further preferably 1,3-benzodioxol-5-yl, 1,4-benzodioxan-6-yl, 2,1,3-benzothiadiazol-4-, -5-yl or 2,1,3-benzoxadiazol-5-yl, azabicyclo[3.2.1]octyl or dibenzofuranyl. The heterocyclic radicals may also be partially or fully hydrogenated. In some embodiments, Het can thus also denote, for example, 2,3-dihydro-2-, -3-, -4- or -5-furyl, 2,5-dihydro-2-, -3-, -4- or 5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-,-3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-, -2-, -3-, -4-, -5-, -6-, -7- or -8-isoquinolyl, 2-, 3-, 5-, 6-, 7- or 8-3,4-dihydro-2H-benzo-1,4-oxazinyl, furthermore preferably 2, 3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydrobenzofuran-5- or 6-yl, 2,3-(2-oxomethylenedioxy)phenyl or also 3,4-dihydro-2H-1,5-benzodioxepin-6- or -7-yl, furthermore preferably 2,3-dihydrobenzofuranyl, 2,3-dihydro-2-oxofuranyl, 3,4-dihydro-2-oxo-1H-quinazolinyl, 2,3-dihydrobenzoxazolyl, 2-oxo-2,3-di-hydrobenzoxazolyl, 2,3-dihydrobenzimidazolyl, 1,3-dihydroindole, 2-oxo-1,3-dihydroindole or 2-oxo-2,3-dihydrobenzimidazolyl. In some embodiments, Het¹ preferably is a monocyclic aromatic heterocycle having 1 to 4 N, O and/or S atoms, which is unsubstituted or mono- or disubstituted by Hal and/or A. In some embodiments, Het¹ furthermore preferably is furyl, thienyl, pyrrolyl, imidazolyl, pyrazolyl, oxazolyl, isoxazolyl, thiazolyl, isothiazolyl, pyridyl, pyrimidinyl, tri-azolyl, tetrazolyl, oxadiazolyl, thiadiazolyl, pyridazinyl, pyrazinyl, each of which is unsubstituted or mono- or disubstituted by Hal and/or A. In some embodiments, Het¹ furthermore preferably is furyl, thienyl, pyrrolyl, imidazolyl, pyrazolyl, oxazolyl, isoxazolyl, thiazolyl, isothiazolyl, pyridyl, pyrimidinyl, tri-azolyl, tetrazolyl, oxadiazolyl, thiadiazolyl, pyridazinyl, pyrazinyl, tetrahydrofuranyl, [1,3]dioxolanyl, pyrrolidinyl, piperidinyl or morpholinyl, each of which is unsubstituted or mono- or disubstituted by Hal, A and/or ═O. In some embodiments, Het¹ particularly preferably is pyridyl, pyrazolyl, tetrahydrofuranyl or [1,3]dioxolanyl, each of which is unsubstituted or mono- or disubstituted by A.

In some embodiments, Hal preferably is F, Cl or Br, but also I, particularly preferably F or Cl.

Throughout the invention, all radicals which occur more than once may be identical or different, i.e. are independent of one another.

The compounds of Formula (XXXV) may have one or more chiral centres and can therefore occur in various stereoisomeric forms. The Formula (XXXV) encompasses all these forms.

Accordingly, the invention relates, in particular, to the compounds of the Formula (XXXV) in which at least one of the said radicals has one of the preferred meanings indicated above. Some preferred groups of compounds may be expressed by the following sub-formulae (XXXV-A) to (XXXV-N), which conform to the Formula (XXXV) and in which the radicals not designated in greater detail have the meaning indicated for the Formula (XXXV), but in which in Formula (XXXV-A) X¹ is CR⁸ or N; X² is N; X³ is CR⁸; in Formula (XXXV-B) R¹ is H or F; in Formula (XXXV-C) R² is H; in Formula (XXXV-D) R² is A or [C(R³)₂]ₙCyc; in Formula (XXXV-E) R⁴ is H; in Formula (XXXV-F) R³ is H or methyl; in Formula (XXXV-G) A is unbranched or branched alkyl with 1-6 C-atoms; in Formula (XXXV-H) Ar is phenyl, which is unsubstituted or mono-, di- or trisubstituted by Hal, A, Het¹, [C(R³)₂]ₚOR³, [C(R³)₂]ₚCOOR³, OCH₂Cyc, CONR³₂ and/or CN; in Formula (XXXV-I) Het is a mono- or bicyclic aromatic heterocycle having 1 to 4 N, O and/or S atoms, which is unsubstituted or mono- or disubstituted by Hal, A and/or ═O; in Formula (XXXV-J) Het is furyl, thienyl, pyrrolyl, imidazolyl, pyrazolyl, oxazolyl, isoxazolyl, thiazolyl, isothiazolyl, pyridyl, pyrimidinyl, triazolyl, tetrazolyl, oxadiazolyl, thiadiazolyl, pyridazinyl, pyrazinyl, benzoxazolyl, benzothiazolyl, benzimidazolyl, benzotriazolyl, indolyl, benzo-1,3-dioxolyl, benzodioxanyl, benzothiadiazolyl, indazolyl, benzofuranyl, quinolyl, isoquinolyl, pyrrolo[2,3-b]pyridyl, oxazolo[5,4-bjpyridyl, imidazo[1,2-a]pyrimidinyl, 2,3-dihydro-indolyl, 2,3-dihydro-benzimidazolyl, imidazo[1,2-a]pyridyl, pyrrolo[3,2-b]pyridyl or oxazolo[5,4-c]pyridyl, each of which is unsubstituted or mono- or disubstituted by Hal, A and/or ═O; in Formula (XXXV-K) Het is a monocyclic aromatic heterocycle having 1 to 4 N, O and/or S atoms, which is unsubstituted or mono- or disubstituted by Hal and/or A; in Formula (XXXV-L) Het is furyl, thienyl, pyrrolyl, imidazolyl, pyrazolyl, oxazolyl, isoxazolyl, thiazolyl, isothiazolyl, pyridyl, pyrimidinyl, triazolyl, tetrazolyl, oxadiazolyl, thiadiazolyl, pyridazinyl, pyrazinyl, tetrahydrofuranyl, [1,3]dioxolanyl, pyrrolidinyl, piperidinyl or morpholinyl, each of which is unsubstituted or mono- or disubstituted by Hal, A and/or ═O; in Formula (XXXV-M) R¹ is Ar or Het; Y is —CO—W or —NR⁴CO—W¹; W is NR²R²; W¹ is A, Cyc, Het¹, CH₂Cyc or CH(OH)CH₂OH; R¹ is H or F; R², R^{2′} each, independently of one another, denote H, A or [C(R³)₂]ₙCyc; X¹, X², X³ each, independently of one another, denote CR⁸ or N; X⁴ is CR⁸ or N; X⁵ is CR⁸ or N; R⁴ is H; A is unbranched or branched alkyl with 1-6 C-atoms; Cyc is cycloalkyl with 3-7 C-atoms, which is unsubstituted or monosubstituted by A; A′ is unbranched or branched alkyl with 1-6 C-atoms; Ar is phenyl, which is unsubstituted or mono-, di- or trisubstituted by Hal, A, Het¹, [C(R³)₂]ₚOR³, [C(R³)₂]ₚCOOR³, OCH₂Cyc, CONR³₂ and/or CN; R³ is H or unbranched or branched alkyl with 1-6 C— atoms; R⁸ is H or A′; Het is a mono- or bicyclic aromatic heterocycle having 1 to 4 N, O and/or S atoms, which is unsubstituted or mono- or disubstituted by Hal, A and/or ═O; Het¹ is furyl, thienyl, pyrrolyl, imidazolyl, pyrazolyl, oxazolyl, isoxazolyl, thiazolyl, isothiazolyl, pyridyl, pyrimidinyl, triazolyl, tetrazolyl, oxadiazolyl, thiadiazolyl, pyridazinyl, pyrazinyl, tetrahydrofuranyl, [1,3]dioxolanyl, pyrrolidinyl, piperidinyl or morpholinyl, each of which is unsubstituted or mono- or disubstituted by Hal, A and/or ═O; Hal is F, Cl, Br or I; n is 0, 1 or 2; p is 0, 1, 2, 3 or 4; q is 1; and pharmaceutically acceptable salts, tautomers and stereoisomers thereof, including mixtures thereof in all ratios.

In some embodiments, the compound has the structure of one of the following:

| | Compound |
|---|---|
| 1697 | 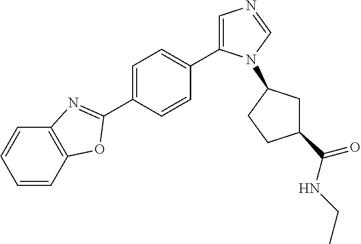 [SMILES: CCNC(=O)[C@H]1CC[C@@H](n2cncc2-c2ccc(-c3nc4ccccc4o3)cc2)C1] |
| 1698 | 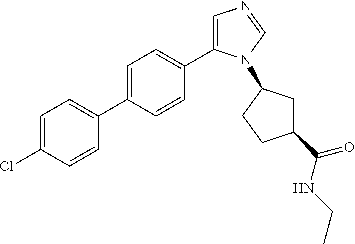 [SMILES: CCNC(=O)[C@H]1CC[C@@H](n2cncc2-c2ccc(-c3ccc(Cl)cc3)cc2)C1] |
| 1699 | 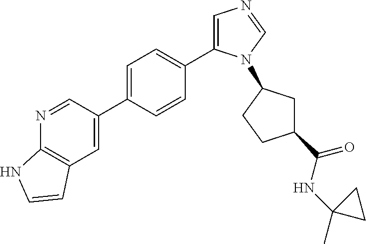 [SMILES: CC1(NC(=O)[C@H]2CC[C@@H](n3cncc3-c3ccc(-c4cnc5[nH]ccc5c4)cc3)C2)CC1] |
| 1700 | 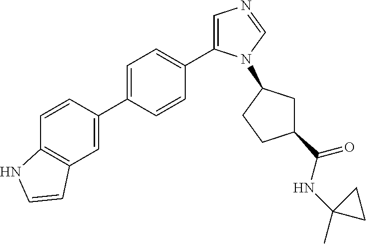 [SMILES: CC1(NC(=O)[C@H]2CC[C@@H](n3cncc3-c3ccc(-c4ccc5[nH]ccc5c4)cc3)C2)CC1] |
| 1701 | 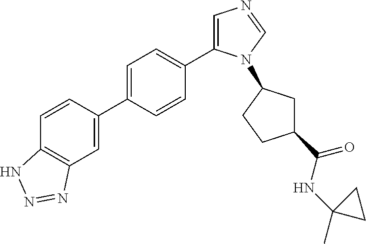 [SMILES: CC1(NC(=O)[C@H]2CC[C@@H](n3cncc3-c3ccc(-c4ccc5[nH]nnc5c4)cc3)C2)CC1] |
| 1702 | 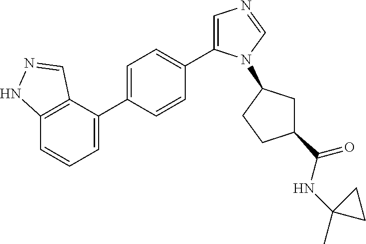 [SMILES: CC1(NC(=O)[C@H]2CC[C@@H](n3cncc3-c3ccc(-c4cccc5[nH]ncc45)cc3)C2)CC1] |
| 1703 | 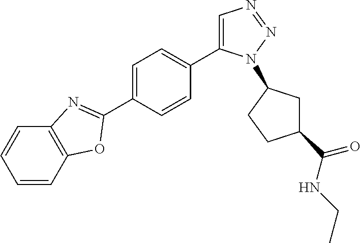 [SMILES: CCNC(=O)[C@H]1CC[C@@H](n2nncc2-c2ccc(-c3nc4ccccc4o3)cc2)C1] |
| 1704 | 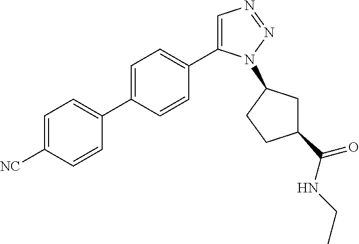 [SMILES: CCNC(=O)[C@H]1CC[C@@H](n2nncc2-c2ccc(-c3ccc(C#N)cc3)cc2)C1] |
| 1705 | 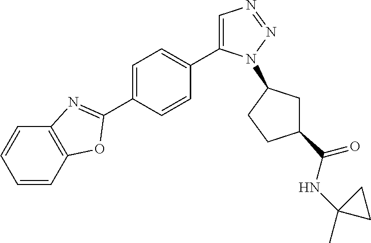 [SMILES: CC1(NC(=O)[C@H]2CC[C@@H](n3nncc3-c3ccc(-c4nc5ccccc5o4)cc3)C2)CC1] |
| 1706 | 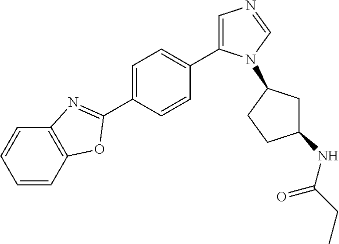 [SMILES: CCC(=O)N[C@H]1CC[C@@H](n2cncc2-c2ccc(-c3nc4ccccc4o3)cc2)C1] |
| 1707 | 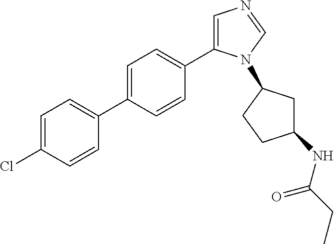 [SMILES: CCC(=O)N[C@H]1CC[C@@H](n2cncc2-c2ccc(-c3ccc(Cl)cc3)cc2)C1] |
| 1708 | 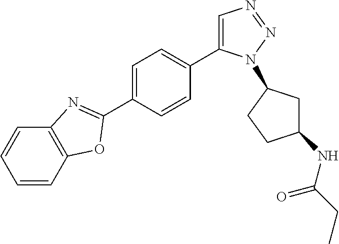 [SMILES: CCC(=O)N[C@H]1CC[C@@H](n2nncc2-c2ccc(-c3nc4ccccc4o3)cc2)C1] |
| 1709 | 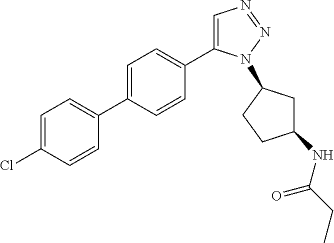 [SMILES: CCC(=O)N[C@H]1CC[C@@H](n2nncc2-c2ccc(-c3ccc(Cl)cc3)cc2)C1] |
| 1710 | 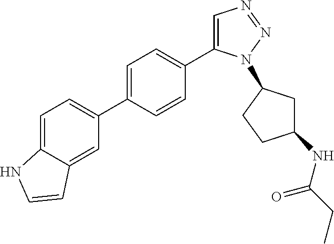 [SMILES: CCC(=O)N[C@H]1CC[C@@H](n2nncc2-c2ccc(-c3ccc4[nH]ccc4c3)cc2)C1] |
| 1711 | 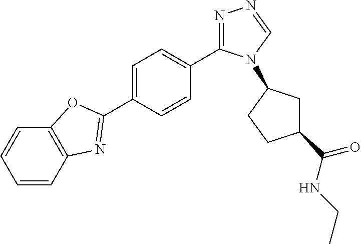 [SMILES: CCNC(=O)[C@H]1CC[C@@H](n2cnnc2-c2ccc(-c3nc4ccccc4o3)cc2)C1] |
| 1712 | 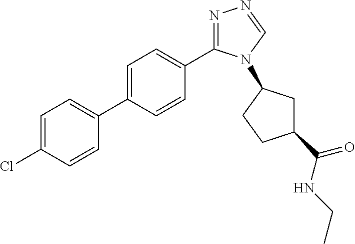 [SMILES: CCNC(=O)[C@H]1CC[C@@H](n2cnnc2-c2ccc(-c3ccc(Cl)cc3)cc2)C1] |
| 1713 | 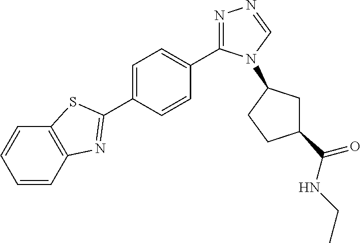 [SMILES: CCNC(=O)[C@H]1CC[C@@H](n2cnnc2-c2ccc(-c3nc4ccccc4s3)cc2)C1] |
| 1714 | 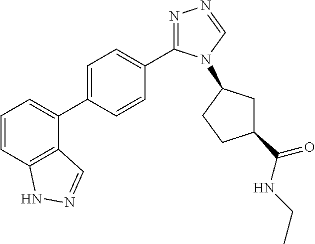 [SMILES: CCNC(=O)[C@H]1CC[C@@H](n2cnnc2-c2ccc(-c3cccc4[nH]ncc34)cc2)C1] |
| 1715 | 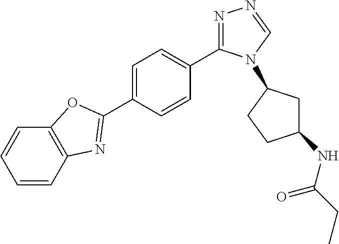 [SMILES: CCC(=O)N[C@H]1CC[C@@H](n2cnnc2-c2ccc(-c3nc4ccccc4o3)cc2)C1] |
| 1716 | 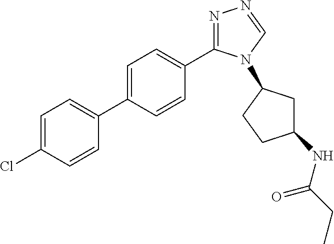 [SMILES: CCC(=O)N[C@H]1CC[C@@H](n2cnnc2-c2ccc(-c3ccc(Cl)cc3)cc2)C1] |
| 1717 | 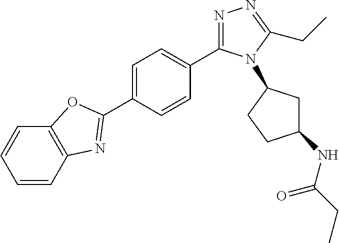 [SMILES: CCC(=O)N[C@H]1CC[C@@H](n2c(CC)nnc2-c2ccc(-c3nc4ccccc4o3)cc2)C1] |
| 1718 | 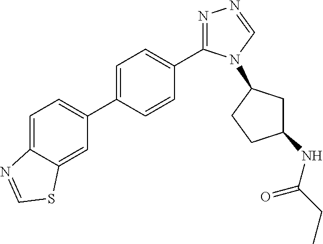 [SMILES: CCC(=O)N[C@H]1CC[C@@H](n2cnnc2-c2ccc(-c3ccc4ncsc4c3)cc2)C1] |
| 1719 | 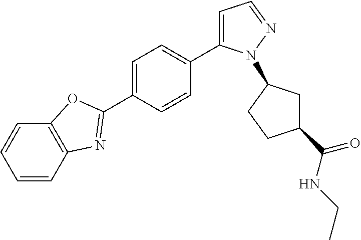 [SMILES: CCNC(=O)[C@H]1CC[C@@H](n2nccc2-c2ccc(-c3nc4ccccc4o3)cc2)C1] |
| 1720 | 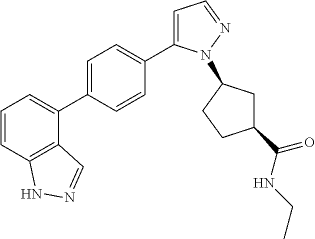 [SMILES: CCNC(=O)[C@H]1CC[C@@H](n2nccc2-c2ccc(-c3cccc4[nH]ncc34)cc2)C1] |
| 1721 | 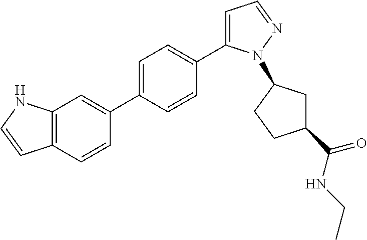 [SMILES: CCNC(=O)[C@H]1CC[C@@H](n2nccc2-c2ccc(-c3ccc4cc[nH]c4c3)cc2)C1] |
| 1722 | 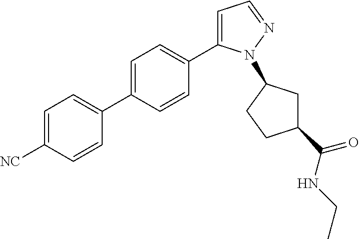 [SMILES: CCNC(=O)[C@H]1CC[C@@H](n2nccc2-c2ccc(-c3ccc(C#N)cc3)cc2)C1] |
| 1723 | 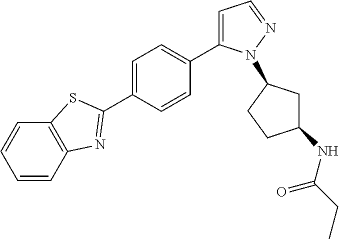 [SMILES: CCC(=O)N[C@H]1CC[C@@H](n2nccc2-c2ccc(-c3nc4ccccc4s3)cc2)C1] |
| 1724 | 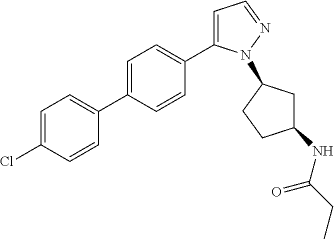 [SMILES: CCC(=O)N[C@H]1CC[C@@H](n2nccc2-c2ccc(-c3ccc(Cl)cc3)cc2)C1] |
| 1725 | 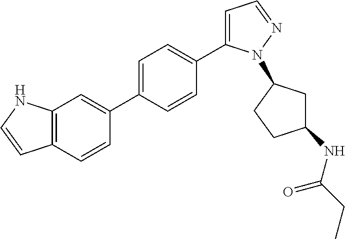 [SMILES: CCC(=O)N[C@H]1CC[C@@H](n2nccc2-c2ccc(-c3ccc4cc[nH]c4c3)cc2)C1] |
| 1726 | 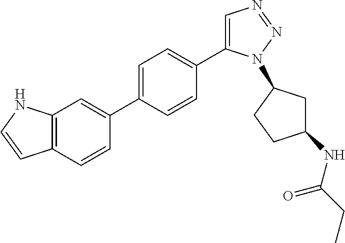 [SMILES: CCC(=O)N[C@H]1CC[C@@H](n2nncc2-c2ccc(-c3ccc4cc[nH]c4c3)cc2)C1] |
| 1727 | 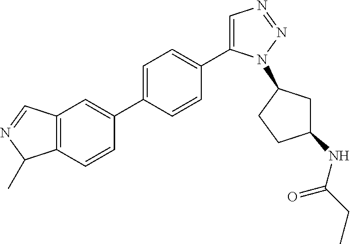 [SMILES: CCC(=O)N[C@H]1CC[C@@H](n2nncc2-c2ccc(-c3ccc4c(c3)C=NC4C)cc2)C1] |
| 1728 | 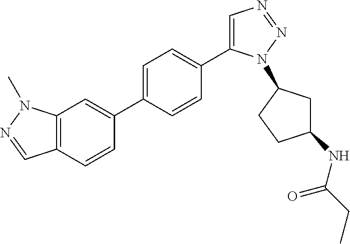 [SMILES: CCC(=O)N[C@H]1CC[C@@H](n2nncc2-c2ccc(-c3ccc4cnn(C)c4c3)cc2)C1] |
| 1729 | 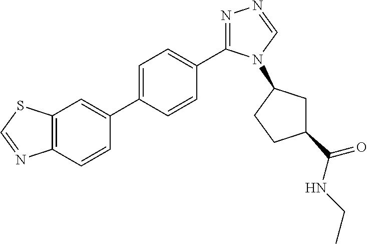 [SMILES: CCNC(=O)[C@H]1CC[C@@H](n2cnnc2-c2ccc(-c3ccc4ncsc4c3)cc2)C1] |
| 1730 | 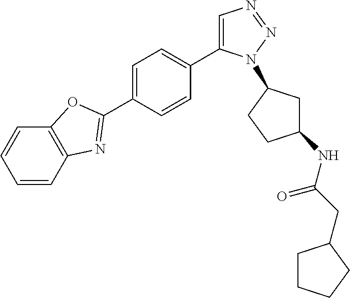 [SMILES: O=C(CC1CCCC1)N[C@H]1CC[C@@H](n2nncc2-c2ccc(-c3nc4ccccc4o3)cc2)C1] |
| 1731 | 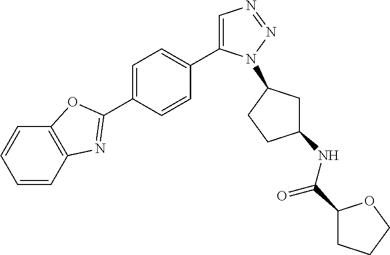 [SMILES: O=C(N[C@H]1CC[C@@H](n2nncc2-c2ccc(-c3nc4ccccc4o3)cc2)C1)[C@@H]1CCCO1] |
| 1732 | 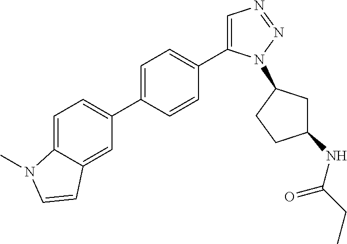 [SMILES: CCC(=O)N[C@H]1CC[C@@H](n2nncc2-c2ccc(-c3ccc4c(ccn4C)c3)cc2)C1] |
| 1733 | 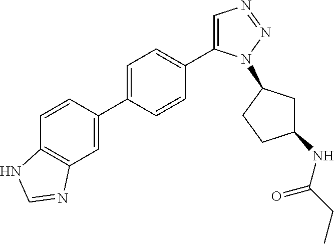 [SMILES: CCC(=O)N[C@H]1CC[C@@H](n2nncc2-c2ccc(-c3ccc4[nH]cnc4c3)cc2)C1] |
| 1734 | 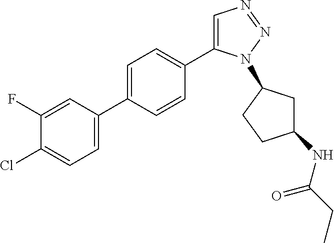 [SMILES: CCC(=O)N[C@H]1CC[C@@H](n2nncc2-c2ccc(-c3ccc(Cl)c(F)c3)cc2)C1] |
| 1735 | 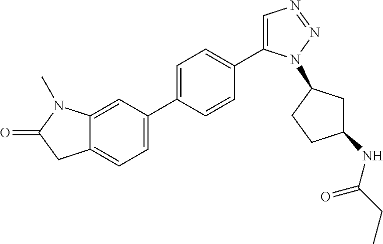 [SMILES: CCC(=O)N[C@H]1CC[C@@H](n2nncc2-c2ccc(-c3ccc4c(c3)N(C)C(=O)C4)cc2)C1] |
| 1736 | 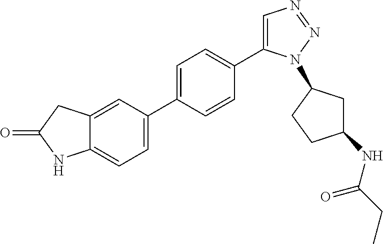 [SMILES: CCC(=O)N[C@H]1CC[C@@H](n2nncc2-c2ccc(-c3ccc4c(c3)CC(=O)N4)cc2)C1] |
| 1737 | 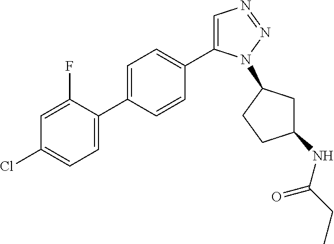 [SMILES: CCC(=O)N[C@H]1CC[C@@H](n2nncc2-c2ccc(-c3ccc(Cl)cc3F)cc2)C1] |
| 1738 | 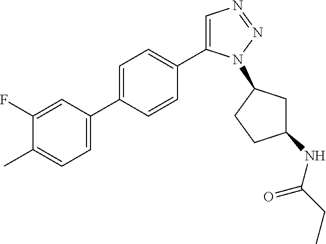 [SMILES: CCC(=O)N[C@H]1CC[C@@H](n2nncc2-c2ccc(-c3ccc(C)c(F)c3)cc2)C1] |
| 1739 | 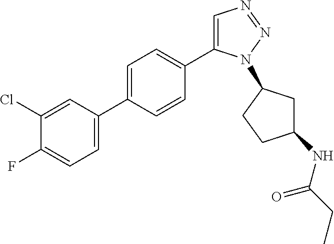 [SMILES: CCC(=O)N[C@H]1CC[C@@H](n2nncc2-c2ccc(-c3ccc(F)c(Cl)c3)cc2)C1] |
| 1740 | 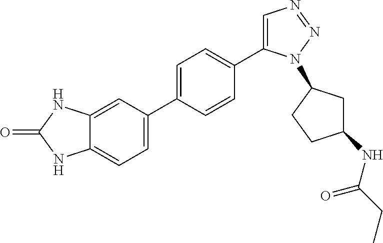 [SMILES: CCC(=O)N[C@H]1CC[C@@H](n2nncc2-c2ccc(-c3ccc4[nH]c(=O)[nH]c4c3)cc2)C1] |
| 1741 | 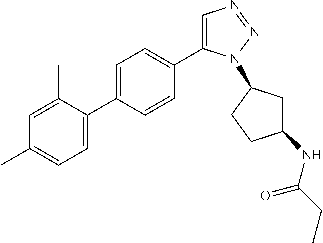 [SMILES: CCC(=O)N[C@H]1CC[C@@H](n2nncc2-c2ccc(-c3ccc(C)cc3C)cc2)C1] |
| 1742 | 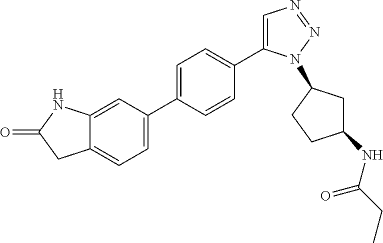 [SMILES: CCC(=O)N[C@H]1CC[C@@H](n2nncc2-c2ccc(-c3ccc4c(c3)NC(=O)C4)cc2)C1] |
| 1743 | 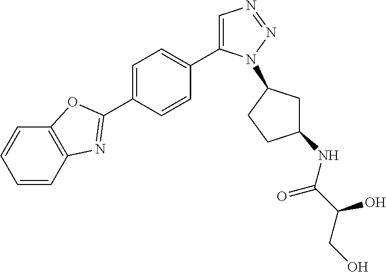 [SMILES: O=C(N[C@H]1CC[C@@H](n2nncc2-c2ccc(-c3nc4ccccc4o3)cc2)C1)[C@@H](O)CO] |
| 1744 | 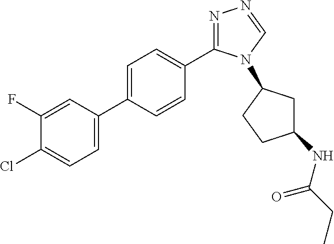 [SMILES: CCC(=O)N[C@H]1CC[C@@H](n2cnnc2-c2ccc(-c3ccc(Cl)c(F)c3)cc2)C1] |
| 1745 | 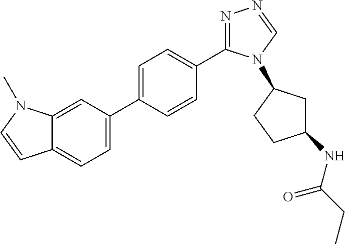 [SMILES: CCC(=O)N[C@H]1CC[C@@H](n2cnnc2-c2ccc(-c3ccc4ccn(C)c4c3)cc2)C1] |
| 1746 | 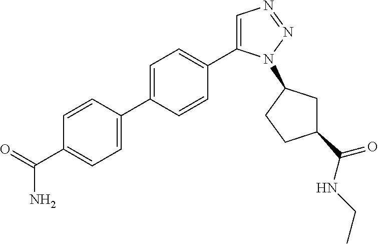 [SMILES: CCNC(=O)[C@H]1CC[C@@H](n2nncc2-c2ccc(-c3ccc(C(N)=O)cc3)cc2)C1] |
| 1747 | 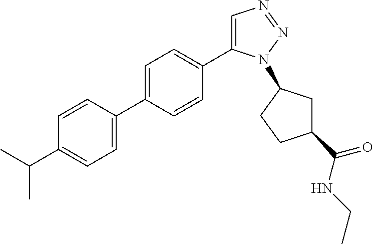 [SMILES: CCNC(=O)[C@H]1CC[C@@H](n2nncc2-c2ccc(-c3ccc(C(C)C)cc3)cc2)C1] |
| 1748 | 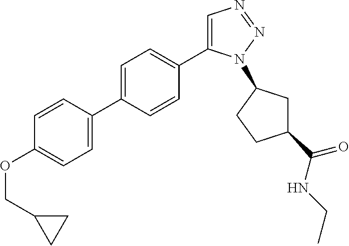 [SMILES: CCNC(=O)[C@H]1CC[C@@H](n2nncc2-c2ccc(-c3ccc(OCC4CC4)cc3)cc2)C1] |
| 1749 | 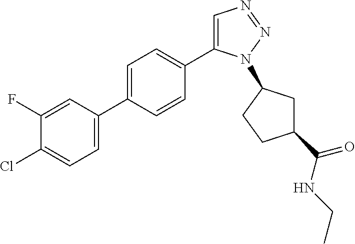 [SMILES: CCNC(=O)[C@H]1CC[C@@H](n2nncc2-c2ccc(-c3ccc(Cl)c(F)c3)cc2)C1] |
| 1750 | 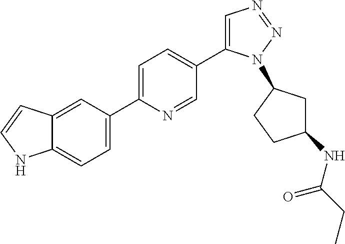 [SMILES: CCC(=O)N[C@H]1CC[C@@H](n2nncc2-c2ccc(-c3ccc4[nH]ccc4c3)nc2)C1] |
| 1751 | 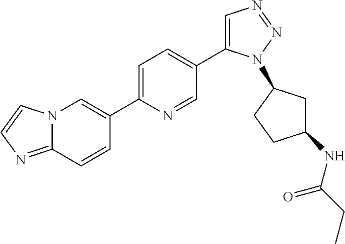 [SMILES: CCC(=O)N[C@H]1CC[C@@H](n2nncc2-c2ccc(-c3ccc4nccn4c3)nc2)C1] |
| 1752 | 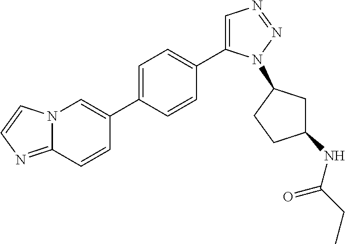 [SMILES: CCC(=O)N[C@H]1CC[C@@H](n2nncc2-c2ccc(-c3ccc4nccn4c3)cc2)C1] |
| 1753 | 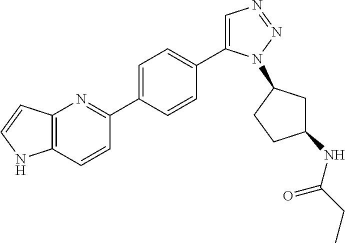 [SMILES: CCC(=O)N[C@H]1CC[C@@H](n2nncc2-c2ccc(-c3ccc4[nH]ccc4n3)cc2)C1] |
| 1754 | 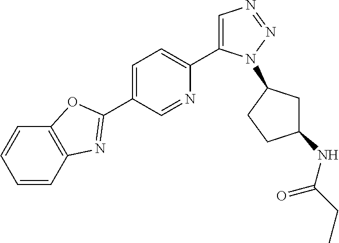 [SMILES: CCC(=O)N[C@H]1CC[C@@H](n2nncc2-c2ccc(-c3nc4ccccc4o3)cn2)C1] |
| 1755 | 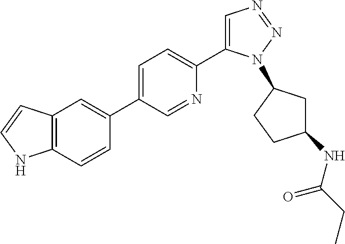 [SMILES: CCC(=O)N[C@H]1CC[C@@H](n2nncc2-c2ccc(-c3ccc4[nH]ccc4c3)cn2)C1] |
| 1756 | 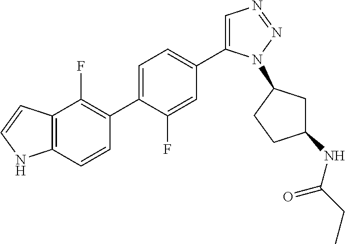 [SMILES: CCC(=O)N[C@H]1CC[C@@H](n2nncc2-c2ccc(-c3ccc4[nH]ccc4c3F)c(F)c2)C1] |
| 1757 | 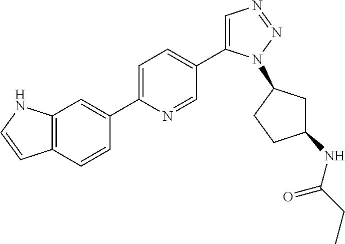 [SMILES: CCC(=O)N[C@H]1CC[C@@H](n2nncc2-c2ccc(-c3ccc4cc[nH]c4c3)nc2)C1] |
| 1758 | 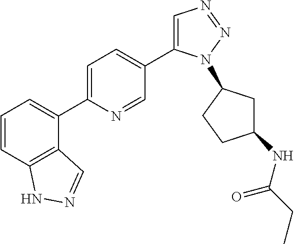 [SMILES: CCC(=O)N[C@H]1CC[C@@H](n2nncc2-c2ccc(-c3cccc4[nH]ncc34)nc2)C1] |
| 1758 | 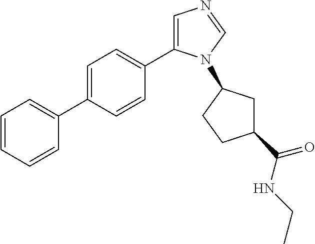 [SMILES: CCNC(=O)[C@H]1CC[C@@H](n2cncc2-c2ccc(-c3ccccc3)cc2)C1] |
| 1759 | 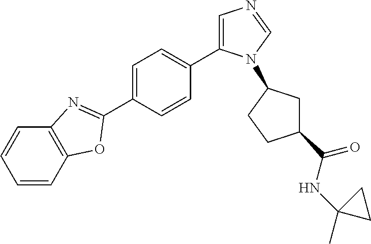 [SMILES: CC1(NC(=O)[C@H]2CC[C@@H](n3cncc3-c3ccc(-c4nc5ccccc5o4)cc3)C2)CC1] |
| 1760 | 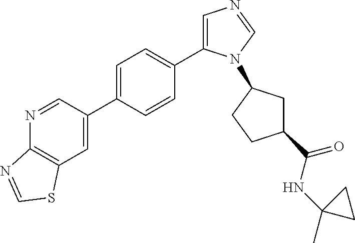 [SMILES: CC1(NC(=O)[C@H]2CC[C@@H](n3cncc3-c3ccc(-c4cnc5ncsc5c4)cc3)C2)CC1] |
| 1761 | 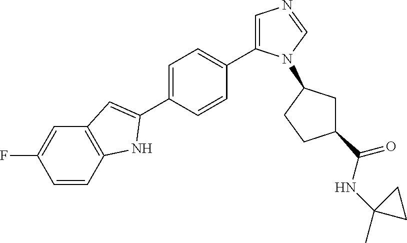 [SMILES: CC1(NC(=O)[C@H]2CC[C@@H](n3cncc3-c3ccc(-c4cc5cc(F)ccc5[nH]4)cc3)C2)CC1] |
| 1762 | 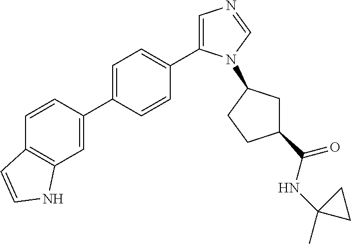 [SMILES: CC1(NC(=O)[C@H]2CC[C@@H](n3cncc3-c3ccc(-c4ccc5cc[nH]c5c4)cc3)C2)CC1] |
| 1763 | 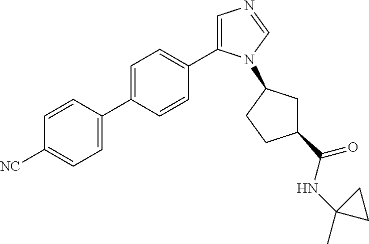 [SMILES: CC1(NC(=O)[C@H]2CC[C@@H](n3cncc3-c3ccc(-c4ccc(C#N)cc4)cc3)C2)CC1] |
| 1764 | 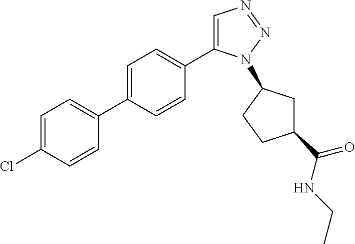 [SMILES: CCNC(=O)[C@H]1CC[C@@H](n2nncc2-c2ccc(-c3ccc(Cl)cc3)cc2)C1] |
| 1765 | 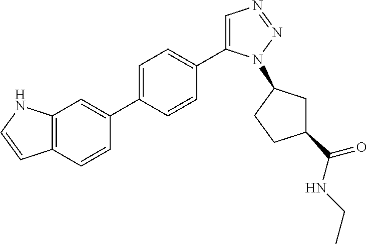 [SMILES: CCNC(=O)[C@H]1CC[C@@H](n2nncc2-c2ccc(-c3ccc4cc[nH]c4c3)cc2)C1] |
| 1766 | 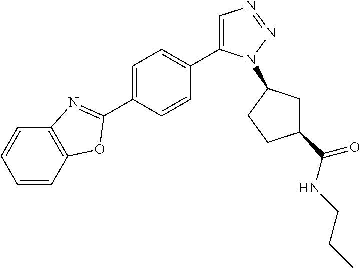 [SMILES: CCCNC(=O)[C@H]1CC[C@@H](n2nncc2-c2ccc(-c3nc4ccccc4o3)cc2)C1] |
| 1767 | 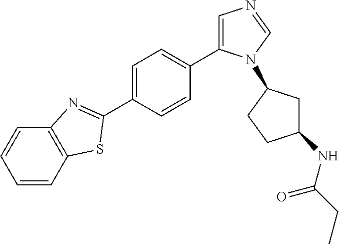 [SMILES: CCC(=O)N[C@H]1CC[C@@H](n2cncc2-c2ccc(-c3nc4ccccc4s3)cc2)C1] |
| 1768 | 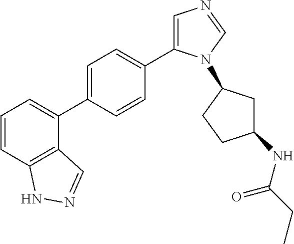 [SMILES: CCC(=O)N[C@H]1CC[C@@H](n2cncc2-c2ccc(-c3cccc4[nH]ncc34)cc2)C1] |
| 1769 | 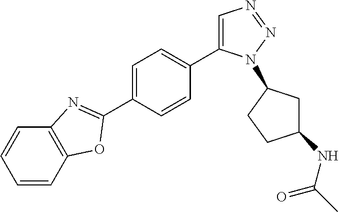 [SMILES: CC(=O)N[C@H]1CC[C@@H](n2nncc2-c2ccc(-c3nc4ccccc4o3)cc2)C1] |
| 1770 | 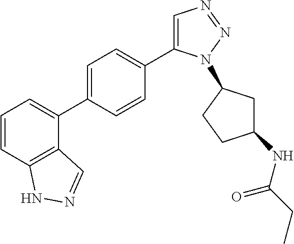 [SMILES: CCC(=O)N[C@H]1CC[C@@H](n2nncc2-c2ccc(-c3cccc4[nH]ncc34)cc2)C1] |
| 1771 | 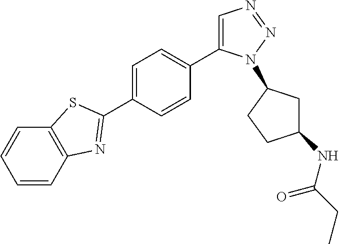 [SMILES: CCC(=O)N[C@H]1CC[C@@H](n2nncc2-c2ccc(-c3nc4ccccc4s3)cc2)C1] |
| 1772 | 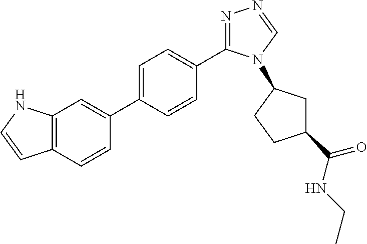 [SMILES: CCNC(=O)[C@H]1CC[C@@H](n2cnnc2-c2ccc(-c3ccc4cc[nH]c4c3)cc2)C1] |
| 1773 | 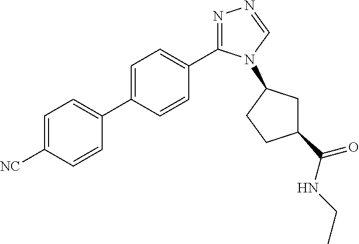 [SMILES: CCNC(=O)[C@H]1CC[C@@H](n2cnnc2-c2ccc(-c3ccc(C#N)cc3)cc2)C1] |
| 1774 | 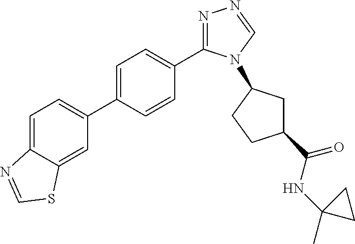 [SMILES: CC1(NC(=O)[C@H]2CC[C@@H](n3cnnc3-c3ccc(-c4ccc5ncsc5c4)cc3)C2)CC1] |
| 1775 | 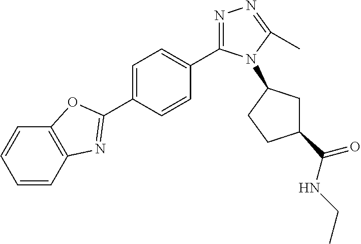 [SMILES: CCNC(=O)[C@H]1CC[C@@H](n2c(C)nnc2-c2ccc(-c3nc4ccccc4o3)cc2)C1] |
| 1776 | 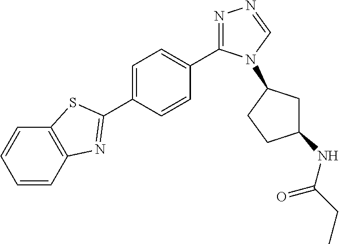 [SMILES: CCC(=O)N[C@H]1CC[C@@H](n2cnnc2-c2ccc(-c3nc4ccccc4s3)cc2)C1] |
| 1777 | 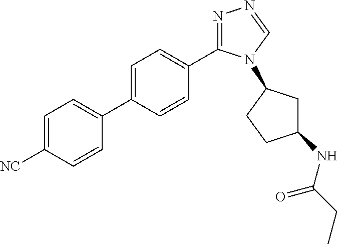 [SMILES: CCC(=O)N[C@H]1CC[C@@H](n2cnnc2-c2ccc(-c3ccc(C#N)cc3)cc2)C1] |
| 1778 | 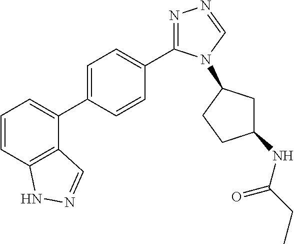 [SMILES: CCC(=O)N[C@H]1CC[C@@H](n2cnnc2-c2ccc(-c3cccc4[nH]ncc34)cc2)C1] |
| 1779 | 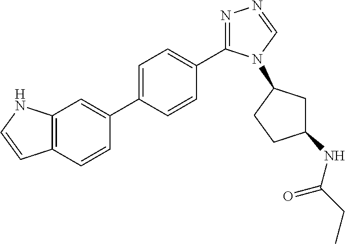 [SMILES: CCC(=O)N[C@H]1CC[C@@H](n2cnnc2-c2ccc(-c3ccc4cc[nH]c4c3)cc2)C1] |
| 1780 | 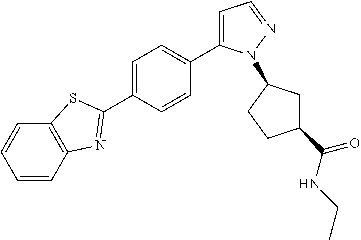 [SMILES: CCNC(=O)[C@H]1CC[C@@H](n2nccc2-c2ccc(-c3nc4ccccc4s3)cc2)C1] |
| 1781 | 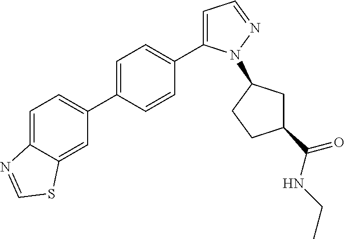 [SMILES: CCNC(=O)[C@H]1CC[C@@H](n2nccc2-c2ccc(-c3ccc4ncsc4c3)cc2)C1] |
| 1782 | 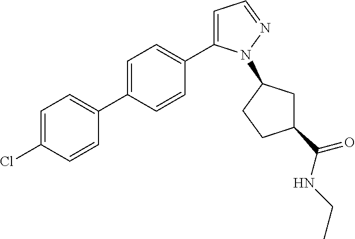 [SMILES: CCNC(=O)[C@H]1CC[C@@H](n2nccc2-c2ccc(-c3ccc(Cl)cc3)cc2)C1] |
| 1783 | 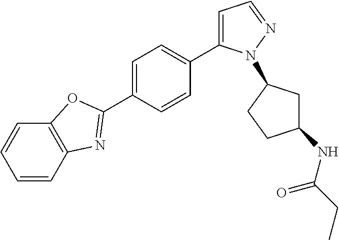 [SMILES: CCC(=O)N[C@H]1CC[C@@H](n2nccc2-c2ccc(-c3nc4ccccc4o3)cc2)C1] |
| 1784 | 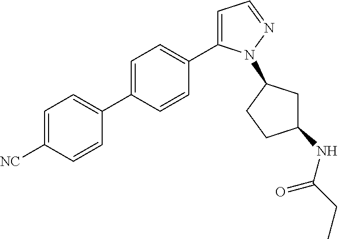 [SMILES: CCC(=O)N[C@H]1CC[C@@H](n2nccc2-c2ccc(-c3ccc(C#N)cc3)cc2)C1] |
| 1785 | 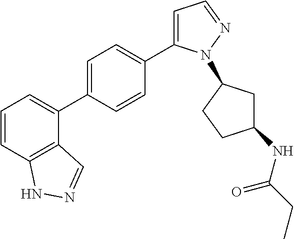 [SMILES: CCC(=O)N[C@H]1CC[C@@H](n2nccc2-c2ccc(-c3cccc4[nH]ncc34)cc2)C1] |
| 1786 | 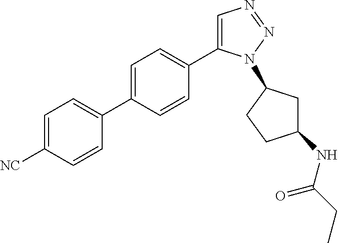 [SMILES: CCC(=O)N[C@H]1CC[C@@H](n2nncc2-c2ccc(-c3ccc(C#N)cc3)cc2)C1] |
| 1787 | 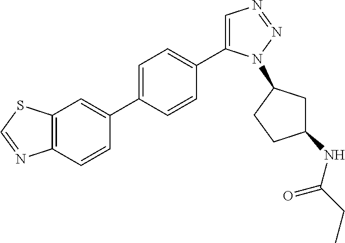 [SMILES: CCC(=O)N[C@H]1CC[C@@H](n2nncc2-c2ccc(-c3ccc4ncsc4c3)cc2)C1] |
| 1788 | 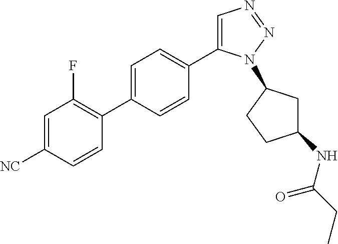 [SMILES: CCC(=O)N[C@H]1CC[C@@H](n2nncc2-c2ccc(-c3ccc(C#N)cc3F)cc2)C1] |
| 1789 | 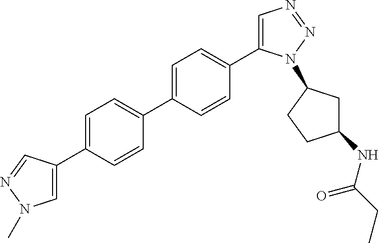 [SMILES: CCC(=O)N[C@H]1CC[C@@H](n2nncc2-c2ccc(-c3ccc(-c4cnn(C)c4)cc3)cc2)C1] |
| 1790 | 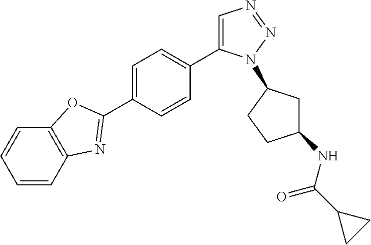 [SMILES: O=C(N[C@H]1CC[C@@H](n2nncc2-c2ccc(-c3nc4ccccc4o3)cc2)C1)C1CC1] |
| 1791 | 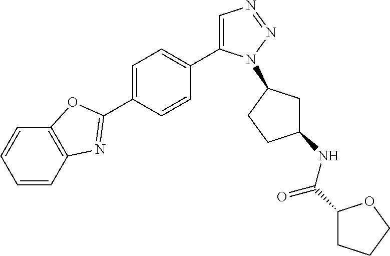 [SMILES: O=C(N[C@H]1CC[C@@H](n2nncc2-c2ccc(-c3nc4ccccc4o3)cc2)C1)[C@H]1CCCO1] |
| 1792 | 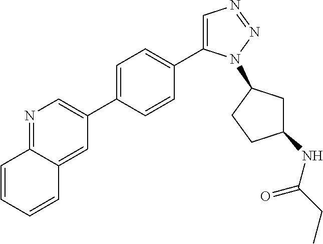 [SMILES: CCC(=O)N[C@H]1CC[C@@H](n2nncc2-c2ccc(-c3cnc4ccccc4c3)cc2)C1] |
| 1793 | 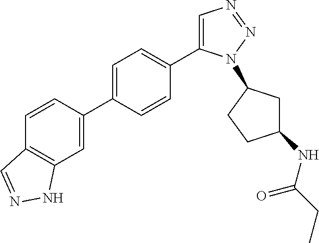 [SMILES: CCC(=O)N[C@H]1CC[C@@H](n2nncc2-c2ccc(-c3ccc4cn[nH]c4c3)cc2)C1] |
| 1794 | 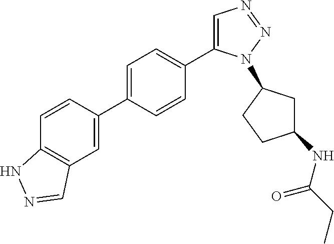 [SMILES: CCC(=O)N[C@H]1CC[C@@H](n2nncc2-c2ccc(-c3ccc4[nH]ncc4c3)cc2)C1] |
| 1795 | 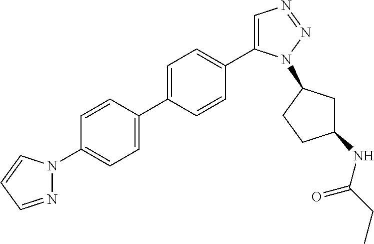 [SMILES: CCC(=O)N[C@H]1CC[C@@H](n2nncc2-c2ccc(-c3ccc(-n4cccn4)cc3)cc2)C1] |
| 1796 | 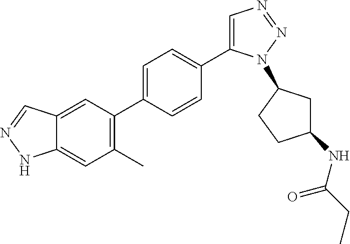 [SMILES: CCC(=O)N[C@H]1CC[C@@H](n2nncc2-c2ccc(-c3cc4cn[nH]c4cc3C)cc2)C1] |
| 1797 | 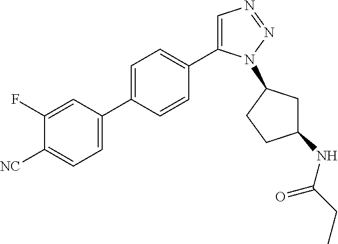 [SMILES: CCC(=O)N[C@H]1CC[C@@H](n2nncc2-c2ccc(-c3ccc(C#N)c(F)c3)cc2)C1] |
| 1798 | 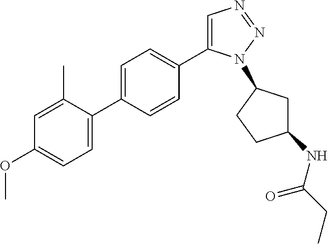 [SMILES: CCC(=O)N[C@H]1CC[C@@H](n2nncc2-c2ccc(-c3ccc(OC)cc3C)cc2)C1] |
| 1799 | 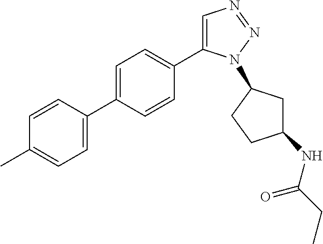 [SMILES: CCC(=O)N[C@H]1CC[C@@H](n2nncc2-c2ccc(-c3ccc(C)cc3)cc2)C1] |
| 1800 | 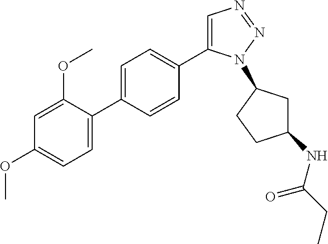 [SMILES: CCC(=O)N[C@H]1CC[C@@H](n2nncc2-c2ccc(-c3ccc(OC)cc3OC)cc2)C1] |
| 1801 | 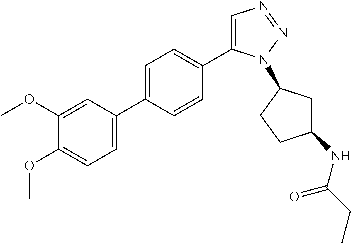 [SMILES: CCC(=O)N[C@H]1CC[C@@H](n2nncc2-c2ccc(-c3ccc(OC)c(OC)c3)cc2)C1] |
| 1802 | 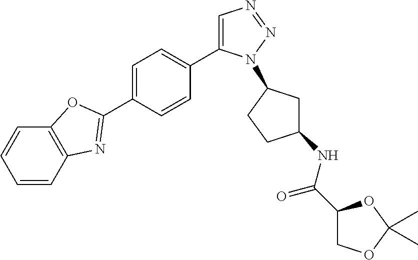 [SMILES: CC1(C)OC[C@@H](C(=O)N[C@H]2CC[C@@H](n3nncc3-c3ccc(-c4nc5ccccc5o4)cc3)C2)O1] |
| 1803 | 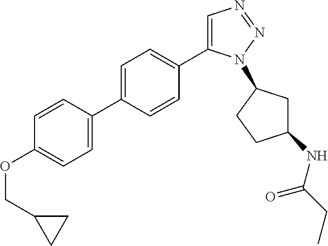 [SMILES: CCC(=O)N[C@H]1CC[C@@H](n2nncc2-c2ccc(-c3ccc(OCC4CC4)cc3)cc2)C1] |
| 1804 | 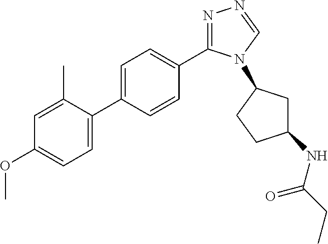 [SMILES: CCC(=O)N[C@H]1CC[C@@H](n2cnnc2-c2ccc(-c3ccc(OC)cc3C)cc2)C1] |
| 1805 | 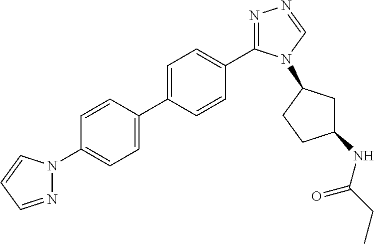 [SMILES: CCC(=O)N[C@H]1CC[C@@H](n2cnnc2-c2ccc(-c3ccc(-n4cccn4)cc3)cc2)C1] |
| 1806 | 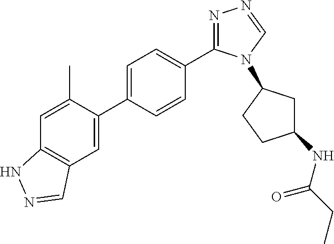 [SMILES: CCC(=O)N[C@H]1CC[C@@H](n2cnnc2-c2ccc(-c3cc4cn[nH]c4cc3C)cc2)C1] |
| 1807 | 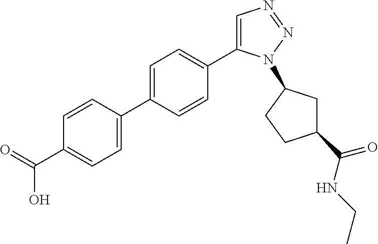 [SMILES: CCNC(=O)[C@H]1CC[C@@H](n2nncc2-c2ccc(-c3ccc(C(=O)O)cc3)cc2)C1] |
| 1808 | 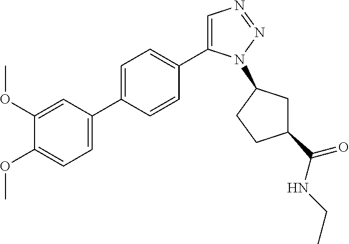 [SMILES: CCNC(=O)[C@H]1CC[C@@H](n2nncc2-c2ccc(-c3ccc(OC)c(OC)c3)cc2)C1] |
| 1809 | 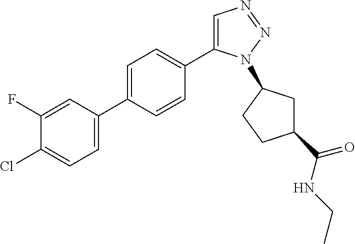 [SMILES: CCNC(=O)[C@H]1CC[C@@H](n2nncc2-c2ccc(-c3ccc(Cl)c(F)c3)cc2)C1] |
| 1810 | 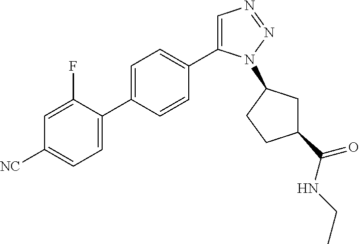 [SMILES: CCNC(=O)[C@H]1CC[C@@H](n2nncc2-c2ccc(-c3ccc(C#N)cc3F)cc2)C1] |
| 1811 | 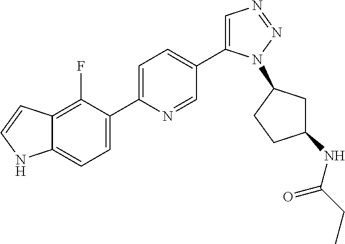 [SMILES: CCC(=O)N[C@H]1CC[C@@H](n2nncc2-c2ccc(-c3ccc4[nH]ccc4c3F)nc2)C1] |
| 1812 | 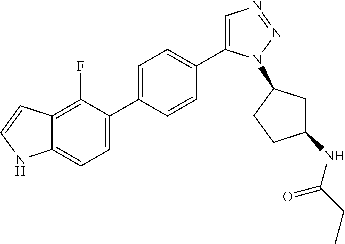 [SMILES: CCC(=O)N[C@H]1CC[C@@H](n2nncc2-c2ccc(-c3ccc4[nH]ccc4c3F)cc2)C1] |
| 1813 | 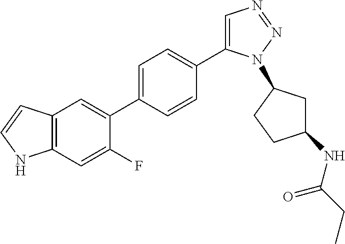 [SMILES: CCC(=O)N[C@H]1CC[C@@H](n2nncc2-c2ccc(-c3cc4cc[nH]c4cc3F)cc2)C1] |
| 1814 | 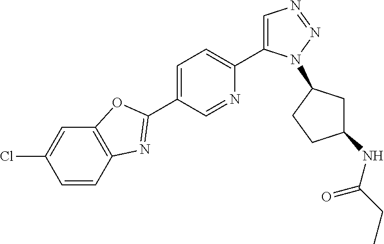 [SMILES: CCC(=O)N[C@H]1CC[C@@H](n2nncc2-c2ccc(-c3nc4ccc(Cl)cc4o3)cn2)C1] |
| 1815 | 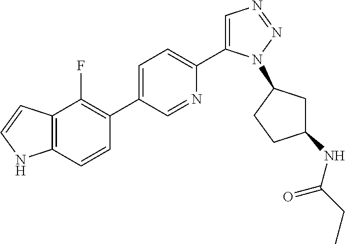 [SMILES: CCC(=O)N[C@H]1CC[C@@H](n2nncc2-c2ccc(-c3ccc4[nH]ccc4c3F)cn2)C1] |
| 1816 | 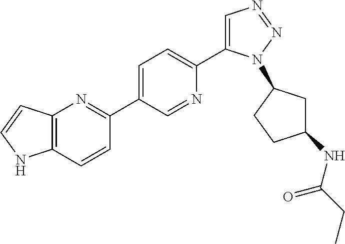 [SMILES: CCC(=O)N[C@H]1CC[C@@H](n2nncc2-c2ccc(-c3ccc4[nH]ccc4n3)cn2)C1] |
| 1817 | 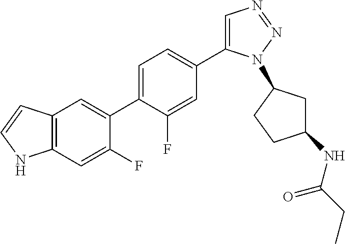 [SMILES: CCC(=O)N[C@H]1CC[C@@H](n2nncc2-c2ccc(-c3cc4cc[nH]c4cc3F)c(F)c2)C1] |
| 1818 | 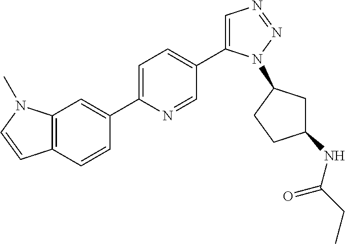 [SMILES: CCC(=O)N[C@H]1CC[C@@H](n2nncc2-c2ccc(-c3ccc4ccn(C)c4c3)nc2)C1] |

In some embodiments, the compound has the structure of Formula (XXXVI):

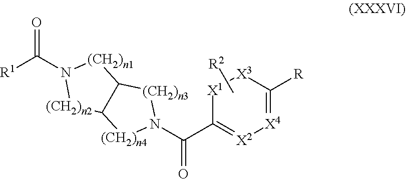

wherein R¹ is A or Cyc, R² is H, F, Cl, Br, OH, CN, NO₂, A′, OA′, SA, SO₂Me, COA′ or CONA′₂, R is Ar or Het, each X¹, X², X³, X⁴ is, independently, CH or N, A is unbranched or branched alkyl with 1-10 C-atoms, wherein two adjacent carbon atoms may form a double bond and/or one or two non-adjacent CH— and/or CH₂— groups may be replaced by N-, O- and/or S-atoms and wherein 1-7H-atoms may be replaced by R⁴, Cyc is cycloalkyl with 3-7 C-atoms, which is unsubstituted or monosubstituted by OH, Hal or A, A′ is unbranched or branched alkyl with 1-6 C-atoms, wherein 1-5H-atoms may be replaced by F, R⁴ is F, Cl, Br, OH, CN, NO₂, A′, OA′, SA′, SO₂Me, COA′ or CONA′₂, Ar is phenyl, which is unsubstituted, or mono-, di-, tri-, tetra- or pentasubstituted by Hal, A, [C(R³)₂]ₚOR³, [C(R³)₂]_{P}NR³₂, NO₂, CN, [C(R³)₂]ₚCOOR³, [C(R³)₂]_{P}NR³₂, NR³₂COA, NR³SO₂A, [C(R³)₂]ₚSO₂NR³₂, S(O)ₙA, O[C(R³)₂]ₘNR³₂, NHCOOA, NHCONR³₂ and/or COA, R³ is H or unbranched or branched alkyl with 1-6 C-atoms, Het is a mono- or bicyclic saturated, unsaturated or aromatic heterocycle having 1 to 4 N, O and/or S atoms, which may be unsubstituted or mono-, di-, tri-, tetra- or pentasubstituted by Hal, A, [C(R³)₂]ₙOR³, [C(R³)₂]ₙNR³₂, SR³, NO₂, CN, COOR³, CONR³₂, NR³COA, NR³SO₂A, SO₂NR³₂, S(O)ₘA, O[C(R³)₂]ₙNR³₂, NHCOOA, NHCONR³₂, CHO, COA, ═S, ═NH, ═NA and/or ═O (carbonyl oxygen), Hal is F, Cl, Br or I, each n1, n2, n3, n4 is, independently, 0, 1 or 2, m is 1, 2 or 3, n is 0, 1 or 2, p is 0, 1, 2, 3 or 4, with the proviso that only one or two of X¹, X², X³, X⁴ denote N, and pharmaceutically acceptable salts, tautomers and stereoisomers thereof, including mixtures thereof in all ratios.

In some embodiments, A is alkyl, this is unbranched (linear) or branched, and has 1, 2, 3, 4, 5, 6, 7, 8, 9 or 10 C atoms. A preferably is methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methyl-propyl, 1,1,2- or 1,2,2-trimethylpropyl, furthermore preferably, for example, trifluoromethyl. A very particularly preferably is alkyl having 1, 2, 3, 4, 5 or 6 C atoms, preferably methyl, ethyl, propyl, isopropyl, butyl, isobutyl, sec-butyl, tert-butyl, pentyl, hexyl, trifluoromethyl, pentafluoroethyl or 1,1,1-trifluoroethyl. In further embodiments, A is preferably CH₂OCH₃, CH₂CH₂OH or CH₂CH₂OCH₃. Cyc is cyclopropyl, cyclobutyl, cyclopentyl, cyclohexyl or cycloheptyl, preferably unsubstituted or monosubstituted by OH, Hal or A.

In some embodiments, R² preferably is H. In some embodiments, R³ preferably is H, methyl, ethyl, propyl, isopropyl, butyl, pentyl or hexyl, particularly preferably H or methyl. In some embodiments, n1, n2, n3, n4 very particularly preferably denote 1.

In some embodiments, Ar is preferably o- m- or p-tolyl, o-, m- or p-ethylphenyl, o-, m- or p-propylphenyl, o-, m- or p-isopropylphenyl, o-, m- or p-tert-butylphenyl, o-, m- or p-hydroxyphenyl, o-, m- or p-nitrophenyl, o-, m- or p-aminophenyl, o-, m- or p-(N-methylamino)phenyl, o-, m- or p-(N-methylaminocarbonyl)phenyl, o-, m- or p-methoxyphenyl, o-, m- or p-ethoxyphenyl, o-, m- or p-ethoxycarbonyl-phenyl, o-, m- or p-(N,N-dimethylamino)phenyl, o-, m- or p-(N,N-dimethyl-aminocarbonyl)phenyl, o-, m- or p-(N-ethylamino)phenyl, o-, m- or p-(N,N-diethylamino)phenyl, o-, m- or p-fluorophenyl, o-, m- or p-bromophenyl, o-, m- or p-chlorophenyl, o-, m- or p-(methylsulfonamido)phenyl, o-, m- or p-(methyl-sulfonyl)phenyl, o-, m- or p-cyanophenyl, o-, m- or p-carboxyphenyl, o-, m- or p-methoxycarbonylphenyl, o-, m- or p-formylphenyl, o-, m- or p-acetylphenyl, o-, m- or p-aminosulfonylphenyl, o-, m- or p-[2-(morpholin-4-yl)ethoxy]phenyl, o-, m- or p-[3-(N,N-diethylamino)propoxy]phenyl, furthermore preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,4- or 2,5-dinitrophenyl, 2,5- or 3,4-dimethoxyphenyl, 3-nitro-4-chlorophenyl, 3-amino-4-chloro-, 2-amino-3-chloro-, 2-amino-4-chloro-, 2-amino-5-chloro- or 2-amino-6-chlorophenyl, 2-nitro-4-N,N-dimethylamino- or 3-nitro-4-N,N-dimethylamino-phenyl, 2,3-diaminophenyl, 2,3,4-, 2,3,5-, 2,3,6-, 2,4,6- or 3,4,5-tri-chlorophenyl, 2,4,6-trimethoxyphenyl, 2-hydroxy-3,5-dichlorophenyl, p-iodo-phenyl, 3,6-dichloro-4-aminophenyl, 4-fluoro-3-chlorophenyl, 2-fluoro-4-bromophenyl, 2,5-difluoro-4-bromophenyl, 3-bromo-6-methoxyphenyl, 3-chloro-6-methoxyphenyl, 3-chloro-4-acetamidophenyl, 3-fluoro-4-methoxyphenyl, 3-amino-6-methylphenyl, 3-chloro-4-acetamidophenyl or 2,5-dimethyl-4-chlorophenyl. In some embodiments, Ar furthermore preferably is phenyl, which is monosubstituted by Hal, A or [C(R²)₂]ₚCOOR².

In some embodiments, irrespective of further substitutions, Het is, for example, 2- or 3-furyl, 2- or 3-thienyl, 1-, 2- or 3-pyrrolyl, 1-, 2, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or 5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, indazolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-iso-quinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, further preferably 1,3-benzodioxol-5-yl, 1,4-benzodioxan-6-yl, 2,1,3-benzothiadiazol-4-, -5-yl or 2,1,3-benzoxadiazol-5-yl, azabicyclo[3.2.1]octyl or dibenzofuranyl. The heterocyclic radicals may also be partially or fully hydrogenated. In some embodiments, irrespective of further substitutions, Het can thus also denote, for example, 2.3-dihydro-2-, -3-, -4- or -5-furyl, 2,5-dihydro-2-, -3-, -4- or 5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-, -2-, -3-, -4-, -5-, -6-, -7- or -8-isoquinolyl, 2-, 3-, 5-, 6-, 7- or 8-3,4-dihydro-2H-benzo-1,4-oxazinyl, furthermore preferably 2,3-methylenedioxyphenyl, 3,4-methylenedioxyphenyl, 2,3-ethylenedioxyphenyl, 3,4-ethylenedioxyphenyl, 3.4-(difluoromethylenedioxy)phenyl, 2,3-dihydrobenzofuran-5- or 6-yl, 2,3-(2-oxomethylenedioxy)phenyl or also 3,4-dihydro-2H-1,5-benzodioxepin-6- or -7-yl, furthermore preferably 2,3-dihydrobenzofuranyl, 2,3-dihydro-2-oxofuranyl, 3,4-dihydro-2-oxo-1H-quinazolinyl, 2,3-dihydrobenzoxazolyl, 2-oxo-2,3-di-hydrobenzoxazolyl, 2,3-dihydrobenzimidazolyl, 1,3-dihydroindole, 2-oxo-1,3-dihydroindole or 2-oxo-2,3-dihydrobenzimidazolyl. In some embodiments, Het preferably is a mono- or bicyclic aromatic heterocycle having 1 to 4 N, O and/or S atoms, which may be unsubstituted or mono- or disubstituted by Hal or A. In some embodiments, Het furthermore preferably is furyl, thienyl, pyrrolyl, imidazolyl, pyrazolyl, oxazolyl, isoxazolyl, thiazolyl, isothiazolyl, pyridyl, pyrimidinyl, triazolyl, tetrazolyl, oxadiazolyl, thiadiazolyl, pyridazinyl, pyrazinyl, benzoxazolyl, benzothiazolyl, benzimidazolyl, benzotriazolyl, indolyl, benzo-1,3-dioxolyl, benzodioxanyl, benzothiadiazolyl, indazolyl, benzofuranyl, quinolyl or isoquinolyl, which may be unsubstituted or mono- or disubstituted by Hal or A. Het very particularly preferably is benzoxazolyl, benzothiazolyl, benzimidazolyl, benzotriazolyl, indolyl, benzo-1,3-dioxolyl, benzodioxanyl, benzothiadiazolyl, indazolyl, benzofuranyl, quinolyl or isoquinolyl.

In some embodiments, Hal preferably is F, Cl or Br, but also I, particularly preferably F or Cl.

Throughout the invention, all radicals which occur more than once may be identical or different, i.e. are independent of one another.

The compounds of the formula I may have one or more chiral centres and can therefore occur in various stereoisomeric forms. The formula I encompasses all these forms.

Accordingly, the invention relates, in particular, to the compounds of the Formula (XXXVI) in which at least one of the said radicals has one of the preferred meanings indicated above. Some preferred groups of compounds may be expressed by the following sub-formulae (XXXVI-A) to (XXXVI-K), which conform to the Formula (XXXVI) and in which the radicals not designated in greater detail have the meaning indicated for the Formula (XXXVI), but in which in Formula (XXXVI-A) X¹, X³ denote CH; X², X⁴ denote N; in Formula (XXXVI-B) X¹, X², X³, X⁴ denote CH; in Formula (XXXVI-C) X¹, X³, X⁴ denote CH; X² is N; in Formula (XXXVI-D) X¹, X², X³ denote CH; X⁴ is N; in Formula (XXXVI-E) X¹, X² denote CH; X³, X⁴ denote N; in Formula (XXXVI-F) X³, X⁴ denote CH; X¹, X² denote N; in Formula (XXXVI-G) R² is H; in Formula (XXXVI-H) Het is a mono- or bicyclic aromatic heterocycle having 1 to 4 N, O and/or S atoms, which may be unsubstituted or mono- or disubstituted by Hal or A; in Formula (XXXVI-I) Het is furyl, thienyl, pyrrolyl, imidazolyl, pyrazolyl, oxazolyl, isoxazolyl, thiazoyl, isothiazolyl, pyridyl, pyrimidinyl, triazolyl, tetrazolyl, oxadiazolyl, thiadiazolyl, pyridazinyl, pyrazinyl, benzoxazolyl, benzothiazolyl, benzimidazolyl, benzotriazolyl, indolyl, benzo-1,3-dioxolyl, benzodioxanyl, benzothiadiazolyl, indazolyl, benzofuranyl, quinolyl or isoquinolyl, which may be unsubstituted or mono- or disubstituted by Hal or A; in Formula (XXXVI-J) Het is benzoxazolyl, benzothiazolyl, benzimidazolyl, benzotriazolyl, indolyl, benzo-1,3-dioxolyl, benzodioxanyl, benzothiadiazolyl, indazolyl, benzofuranyl, quinolyl or isoquinolyl; in Formula (XXXVI-K) R¹ is A or Cyc; R² is H; R is Het; X¹, X², X³, X⁴ each, independently of one another, denote CH or N; A is unbranched or branched alkyl with 1-6 C-atoms, wherein 1-5H-atoms may be replaced by F; Cyc is cycloalkyl with 3-7 C-atoms; Het is furyl, thienyl, pyrrolyl, imidazolyl, pyrazolyl, oxazolyl, isoxazolyl, thiazolyl, isothiazolyl, pyridyl, pyrimidinyl, triazolyl, tetrazolyl, oxadiazolyl, thiadiazolyl, pyridazinyl, pyrazinyl, benzoxazolyl, benzothiazolyl, benzimidazolyl, benzotriazolyl, indolyl, benzo-1,3-dioxolyl, benzodioxanyl, benzothiadiazolyl, indazolyl, benzofuranyl, quinolyl or isoquinolyl, which may be unsubstituted or mono- or disubstituted by Hal or A; Hal is F, Cl, Br or I; n1, n2, n3, n4 each, independently of one another, denote 0, 1 or 2, with the proviso that only one or two of X¹, X², X³, X⁴ denote N, and pharmaceutically acceptable salts, tautomers and stereoisomers thereof, including mixtures thereof in all ratios.

In some embodiments, the compound is one of the following:

| | Compound |
|---|---|
| 1819 | 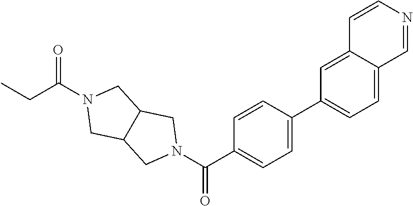 [SMILES: CCC(=O)N1CC2CN(C(=O)c3ccc(-c4ccc5cnccc5c4)cc3)CC2C1] |
| 1820 | 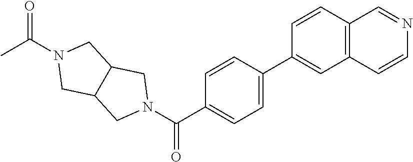 [SMILES: CC(=O)N1CC2CN(C(=O)c3ccc(-c4ccc5cnccc5c4)cc3)CC2C1] |
| 1821 | 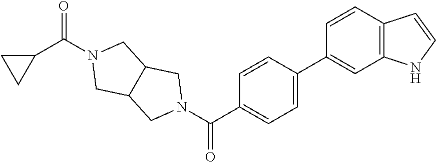 [SMILES: O=C(c1ccc(-c2ccc3cc[nH]c3c2)cc1)N1CC2CN(C(=O)C3CC3)CC2C1] |
| 1822 | 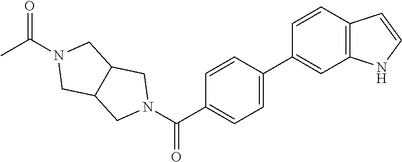 [SMILES: CC(=O)N1CC2CN(C(=O)c3ccc(-c4ccc5cc[nH]c5c4)cc3)CC2C1] |
| 1823 | 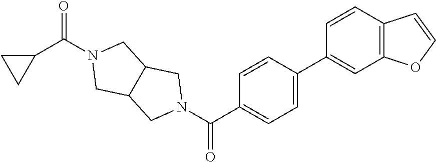 [SMILES: O=C(c1ccc(-c2ccc3ccoc3c2)cc1)N1CC2CN(C(=O)C3CC3)CC2C1] |
| 1824 | 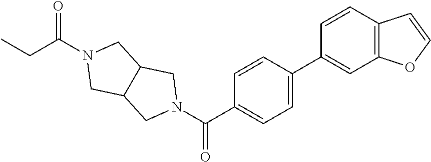 [SMILES: CCC(=O)N1CC2CN(C(=O)c3ccc(-c4ccc5ccoc5c4)cc3)CC2C1] |
| 1825 | 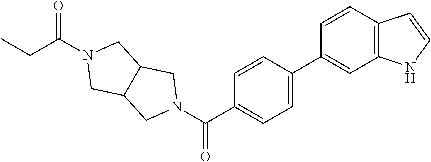 [SMILES: CCC(=O)N1CC2CN(C(=O)c3ccc(-c4ccc5cc[nH]c5c4)cc3)CC2C1] |
| 1826 | 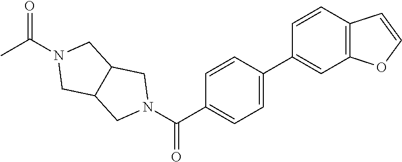 [SMILES: CC(=O)N1CC2CN(C(=O)c3ccc(-c4ccc5ccoc5c4)cc3)CC2C1] |
| 1827 | 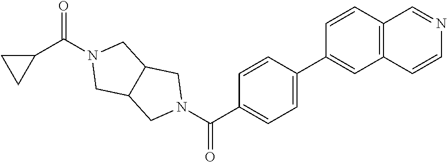 [SMILES: O=C(c1ccc(-c2ccc3cnccc3c2)cc1)N1CC2CN(C(=O)C3CC3)CC2C1] |
| 1828 | |

In some embodiments, the compound has the structure of Formula (XXXVII):

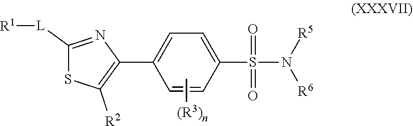

wherein: R¹ is C₁-C₈alkyl, substituted or unsubstituted C₁-C₈haloalkyl, substituted or unsubstituted C₃-C₈cycloalkyl, substituted or unsubstituted C₂-C₈heterocycloalkyl, substituted or unsubstituted aryl, substituted or unsubstituted heteroaryl, substituted or unsubstituted —C₁-C₂alkylene(aryl), or substituted or unsubstituted —C₁-C₂alkylene(heteroaryl); L is absent, C₁-C₄alkylene, —NR⁴—, —CH═N—NR⁴—, —NR⁴C(═O)—, or —C(═O)NR⁴—,—C(═O)NR⁴(C₁-C₄alkylene)-, —NR⁴C(═O)(C₁-C₄alkylene)-, —(C₁-C₄alkylene)C(═O) NR⁴—, —(C₁-C₄ alkylene)NR⁴C(═O)—, —C(═O)NR⁴(C₁-C₄alkylene)O—, —NR⁴C(═O)(C₁-C₄alkylene)O—, —O(C₁-C₄ alkylene)C(═O)NR⁴—, or —O(C₁-C₄alkylene) NR⁴C(═O)—; R² is hydrogen, halogen, —CN, —OH, substituted or unsubstituted C₁-C₆alkyl, substituted or unsubstituted C₁-C₆haloalkyl, substituted or unsubstituted C₁-C₆alkoxy, or substituted or unsubstituted C₁-C₆haloalkoxy; each R³ is independently selected from the group consisting of hydrogen, halogen, —CN, —OH, substituted or unsubstituted C₁-C₆alkyl, substituted or unsubstituted C₁-C₆haloalkyl, substituted or unsubstituted C₁-C₆alkoxy, and substituted or unsubstituted C₁-C₆haloalkoxy; n is 0, 1, 2, 3, or 4; R⁴ is hydrogen, C₁-C₆alkyl, C₁-C₆ haloalkyl, C₃-C₈cycloalkyl, or substituted or unsubstituted aryl; R⁵ and R⁶ are each independently selected from the group consisting of hydrogen, substituted or unsubstituted C₁-C₆alkyl, substituted or unsubstituted C₁-C₆haloalkyl, substituted or unsubstituted C₃-C₈ cycloalkyl, substituted or unsubstituted C₂-C₈heterocycloalkyl, substituted or unsubstituted aryl, substituted or unsubstituted heteroaryl, substituted or unsubstituted —C₁-C₂ alkylene(aryl), and substituted or unsubstituted —C₁-C₂ alkylene (heteroaryl); or R⁵ and R⁶ are taken together with the nitrogen to which they are attached form a substituted or unsubstituted 4-, 5-, 6-, or 7-membered heterocycloalkyl.

Throughout the specification, groups and substituents thereof are chosen by one skilled in the field to provide stable moieties and compounds. For example, in some embodiments, R² is hydrogen, halogen, —CN, —OH, substituted or unsubstituted C₁-C₆alkyl, substituted or unsubstituted C₁-C₆haloalkyl, substituted or unsubstituted C₁-C₆alkoxy, or substituted or unsubstituted C₁-C₆haloalkoxy. In other embodiments, R² is hydrogen, halogen, —CN, C₁-C₆alkyl, or C₁-C₆haloalkyl. In some embodiments, R² is hydrogen, halogen, —CH₃, —CH₂CH₃, —CF₃, or —CH₂CF₃. In some embodiments, R² is hydrogen, halogen, CH₃, or —CF₃. In some embodiments, R² is hydrogen.

In some embodiments, n is 0, 1, 2, 3, or 4. In some embodiments, n is 1, 2, 3, or 4. In some embodiments, n is 0, 1, 2, or 3. In some embodiments, n is 0, 1, or 2. In some embodiments, n is 0, or 1. In some embodiments, n is 0. In some embodiments, n is 1, 2, 3, or 4. In some embodiments, n is 1, 2, or 3. In some embodiments, n is 1, or 2. In some embodiments, n is 1. In some embodiments, R² is hydrogen; R³ is hydrogen; and n is 0.

In some embodiments, the compound has the structure of Formula (XXXVII-A)-(XXXVII-D):

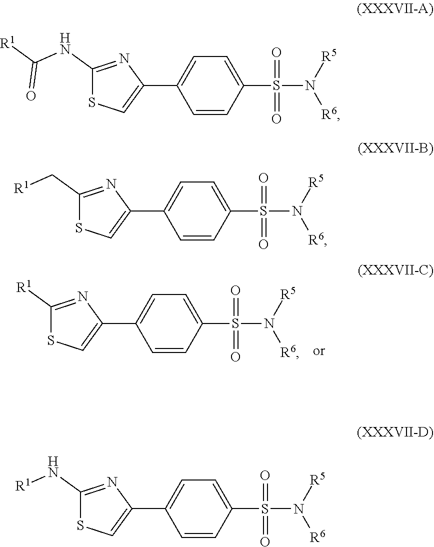

In some embodiments, R¹ is substituted or unsubstituted C₁-C₈alkyl. In some embodiments, R¹ is selected from the group consisting of methyl, ethyl, n-propyl, i-propyl, n-butyl, sec-butyl, i-butyl, t-butyl, 1-ethyl-propyl, n-pentyl, n-hexyl, and n-heptyl. In some embodiments, R¹ is 1-ethyl-propyl or sec-butyl. In some embodiments, R¹ is substituted or unsubstituted aryl. In some embodiments, R¹ is phenyl optionally substituted with halogen, —CN, —OH, C₁-C₆alkyl, C₁-C₆haloalkyl, C₁-C₆alkoxy, or C₁-C₆ haloalkoxy. In some embodiments, R¹ is substituted or unsubstituted C₃-C₈cycloalkyl. In some embodiments, R¹ is selected from the group consisting of cyclopropyl, cyclobutyl, cyclopentyl, and cyclohexyl. In some embodiments, R⁵ and R⁶ are each independently substituted or unsubstituted C₁-C₆ alkyl. In some embodiments, R⁵ and R⁶ are each independently selected from methyl or ethyl. In some embodiments, R⁵ and R⁶ are taken together with the nitrogen to which they are attached form a substituted or unsubstituted 4-, 5-, 6-, or 7-membered heterocycloalkyl. In some embodiments, R⁵ and R⁶ are taken together with the nitrogen to which they are attached form a pyrrolidinyl, morpholinyl, piperidinyl, 4-methylpiperidinyl, 2-methylpiperidinyl, 3-methylpiperidinyl, thiomorpholinyl, piperazinyl, or 4-methylpiperazinyl. In some embodiments, R¹ is sec-butyl; and R⁵ and R⁶ are each ethyl.

In some embodiments, the compound is one of the following:

| | Compound |
|---|---|
| 1829 | 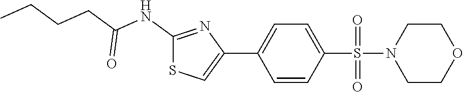 [SMILES: CCCCC(=O)Nc1nc(-c2ccc(S(=O)(=O)N3CCOCC3)cc2)cs1] |
| 1830 | 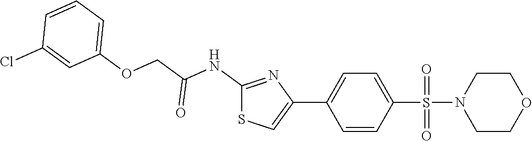 [SMILES: O=C(COc1cccc(Cl)c1)Nc1nc(-c2ccc(S(=O)(=O)N3CCOCC3)cc2)cs1] |
| 1831 | 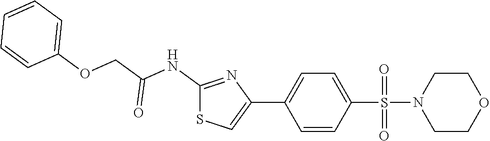 [SMILES: O=C(COc1ccccc1)Nc1nc(-c2ccc(S(=O)(=O)N3CCOCC3)cc2)cs1] |
| 1832 | 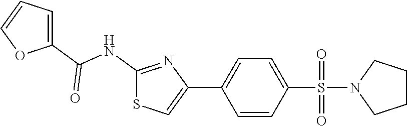 [SMILES: O=C(Nc1nc(-c2ccc(S(=O)(=O)N3CCCC3)cc2)cs1)c1ccco1] |
| 1833 | 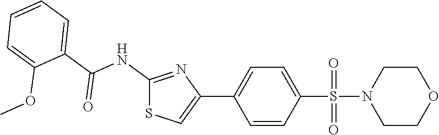 [SMILES: COc1ccccc1C(=O)Nc1nc(-c2ccc(S(=O)(=O)N3CCOCC3)cc2)cs1] |
| 1834 | 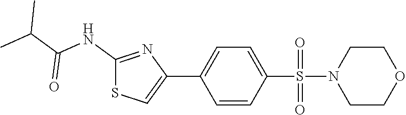 [SMILES: CC(C)C(=O)Nc1nc(-c2ccc(S(=O)(=O)N3CCOCC3)cc2)cs1] |
| 1835 | 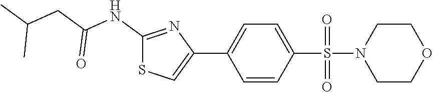 [SMILES: CC(C)CC(=O)Nc1nc(-c2ccc(S(=O)(=O)N3CCOCC3)cc2)cs1] |
| 1836 | 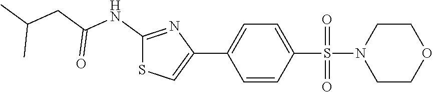 [SMILES: CC(C)CC(=O)Nc1nc(-c2ccc(S(=O)(=O)N3CCOCC3)cc2)cs1] |
| 1837 | 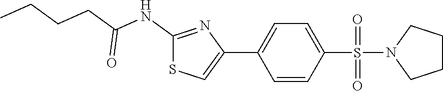 [SMILES: CCCCC(=O)Nc1nc(-c2ccc(S(=O)(=O)N3CCCC3)cc2)cs1] |
| 1838 | 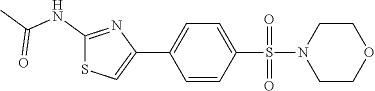 [SMILES: CC(=O)Nc1nc(-c2ccc(S(=O)(=O)N3CCOCC3)cc2)cs1] |
| 1839 | 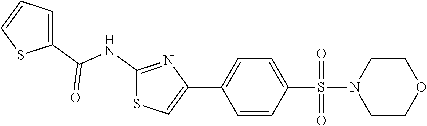 [SMILES: O=C(Nc1nc(-c2ccc(S(=O)(=O)N3CCOCC3)cc2)cs1)c1cccs1] |
| 1840 | 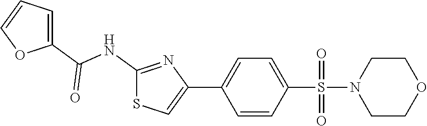 [SMILES: O=C(Nc1nc(-c2ccc(S(=O)(=O)N3CCOCC3)cc2)cs1)c1ccco1] |
| 1841 | 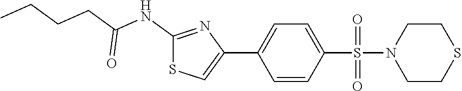 [SMILES: CCCCC(=O)Nc1nc(-c2ccc(S(=O)(=O)N3CCSCC3)cc2)cs1] |
| 1842 | 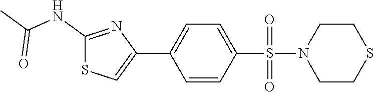 [SMILES: CC(=O)Nc1nc(-c2ccc(S(=O)(=O)N3CCSCC3)cc2)cs1] |
| 1843 | 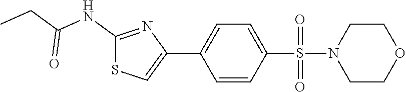 [SMILES: CCC(=O)Nc1nc(-c2ccc(S(=O)(=O)N3CCOCC3)cc2)cs1] |
| 1844 | 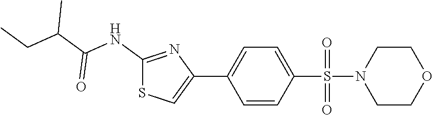 [SMILES: CCC(C)C(=O)Nc1nc(-c2ccc(S(=O)(=O)N3CCOCC3)cc2)cs1] |
| 1845 | 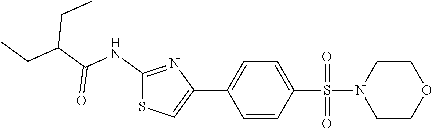 [SMILES: CCC(CC)C(=O)Nc1nc(-c2ccc(S(=O)(=O)N3CCOCC3)cc2)cs1] |
| 1846 | 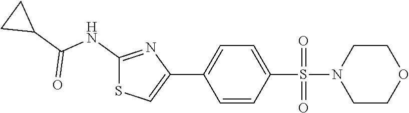 [SMILES: O=C(Nc1nc(-c2ccc(S(=O)(=O)N3CCOCC3)cc2)cs1)C1CC1] |
| 1847 | 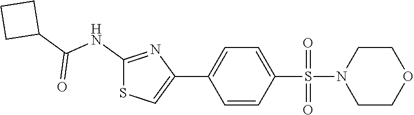 [SMILES: O=C(Nc1nc(-c2ccc(S(=O)(=O)N3CCOCC3)cc2)cs1)C1CCC1] |
| 1848 | 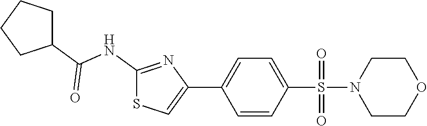 [SMILES: O=C(Nc1nc(-c2ccc(S(=O)(=O)N3CCOCC3)cc2)cs1)C1CCCC1] |
| 1849 | 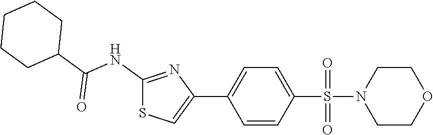 [SMILES: O=C(Nc1nc(-c2ccc(S(=O)(=O)N3CCOCC3)cc2)cs1)C1CCCCC1] |
| 1850 | 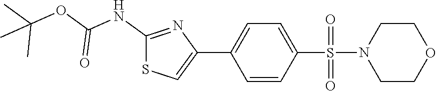 [SMILES: CC(C)(C)OC(=O)Nc1nc(-c2ccc(S(=O)(=O)N3CCOCC3)cc2)cs1] |
| 1851 | 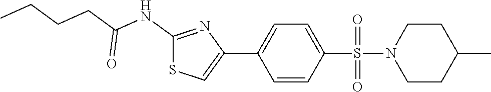 [SMILES: CCCCC(=O)Nc1nc(-c2ccc(S(=O)(=O)N3CCC(C)CC3)cc2)cs1] |
| 1852 | 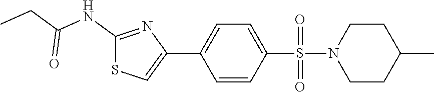 [SMILES: CCC(=O)Nc1nc(-c2ccc(S(=O)(=O)N3CCC(C)CC3)cc2)cs1] |
| 1853 | 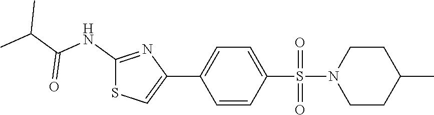 [SMILES: CC1CCN(S(=O)(=O)c2ccc(-c3csc(NC(=O)C(C)C)n3)cc2)CC1] |
| 1854 | 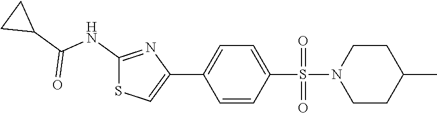 [SMILES: CC1CCN(S(=O)(=O)c2ccc(-c3csc(NC(=O)C4CC4)n3)cc2)CC1] |
| 1855 | 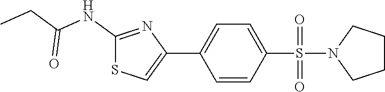 [SMILES: CCC(=O)Nc1nc(-c2ccc(S(=O)(=O)N3CCCC3)cc2)cs1] |
| 1856 | 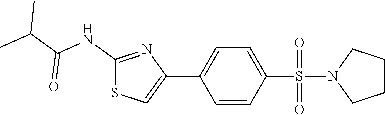 [SMILES: CC(C)C(=O)Nc1nc(-c2ccc(S(=O)(=O)N3CCCC3)cc2)cs1] |
| 1857 | 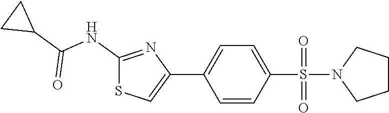 [SMILES: O=C(Nc1nc(-c2ccc(S(=O)(=O)N3CCCC3)cc2)cs1)C1CC1] |
| 1858 | 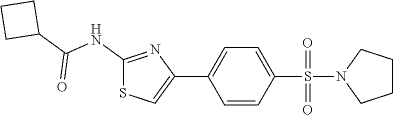 [SMILES: O=C(Nc1nc(-c2ccc(S(=O)(=O)N3CCCC3)cc2)cs1)C1CCC1] |
| 1859 | 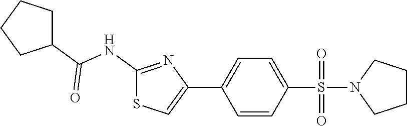 [SMILES: O=C(Nc1nc(-c2ccc(S(=O)(=O)N3CCCC3)cc2)cs1)C1CCCC1] |
| 1860 | 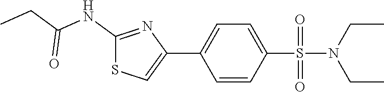 [SMILES: CCC(=O)Nc1nc(-c2ccc(S(=O)(=O)N(CC)CC)cc2)cs1] |
| 1861 | 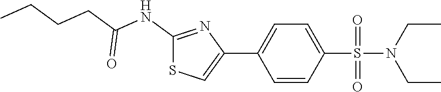 [SMILES: CCCCC(=O)Nc1nc(-c2ccc(S(=O)(=O)N(CC)CC)cc2)cs1] |
| 1862 | 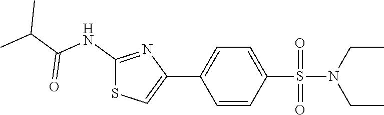 [SMILES: CCN(CC)S(=O)(=O)c1ccc(-c2csc(NC(=O)C(C)C)n2)cc1] |
| 1863 | 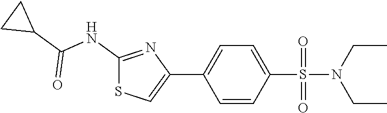 [SMILES: CCN(CC)S(=O)(=O)c1ccc(-c2csc(NC(=O)C3CC3)n2)cc1] |
| 1864 | 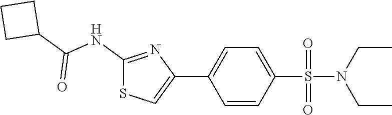 [SMILES: CCN(CC)S(=O)(=O)c1ccc(-c2csc(NC(=O)C3CCC3)n2)cc1] |
| 1865 | 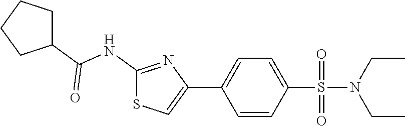 [SMILES: CCN(CC)S(=O)(=O)c1ccc(-c2csc(NC(=O)C3CCCC3)n2)cc1] |
| 1866 | 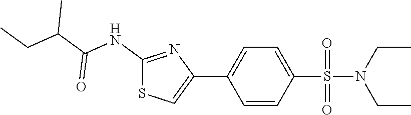 [SMILES: CCC(C)C(=O)Nc1nc(-c2ccc(S(=O)(=O)N(CC)CC)cc2)cs1] |
| 1867 | 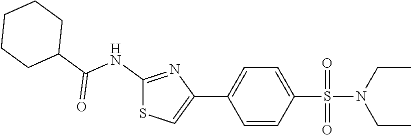 [SMILES: CCN(CC)S(=O)(=O)c1ccc(-c2csc(NC(=O)C3CCCCC3)n2)cc1] |
| 1868 | 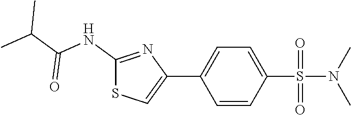 [SMILES: CC(C)C(=O)Nc1nc(-c2ccc(S(=O)(=O)N(C)C)cc2)cs1] |
| 1869 | 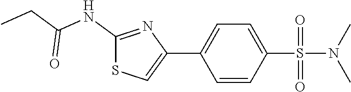 [SMILES: CCC(=O)Nc1nc(-c2ccc(S(=O)(=O)N(C)C)cc2)cs1] |
| 1870 | 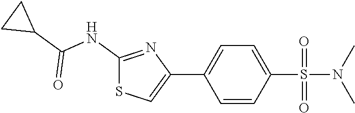 [SMILES: CN(C)S(=O)(=O)c1ccc(-c2csc(NC(=O)C3CC3)n2)cc1] |
| 1871 | 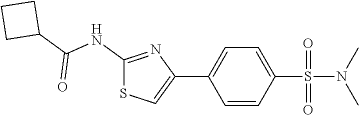 [SMILES: CN(C)S(=O)(=O)c1ccc(-c2csc(NC(=O)C3CCC3)n2)cc1] |
| 1872 | 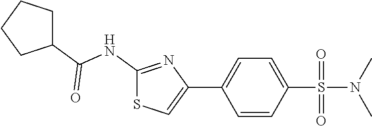 [SMILES: CN(C)S(=O)(=O)c1ccc(-c2csc(NC(=O)C3CCCC3)n2)cc1] |
| 1873 | 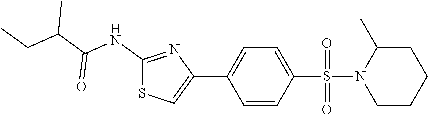 [SMILES: CCC(C)C(=O)Nc1nc(-c2ccc(S(=O)(=O)N3CCCCC3C)cc2)cs1] |
| 1874 | 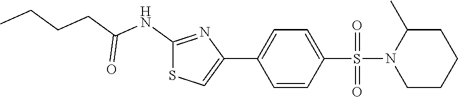 [SMILES: CCCCC(=O)Nc1nc(-c2ccc(S(=O)(=O)N3CCCCC3C)cc2)cs1] |
| 1875 | 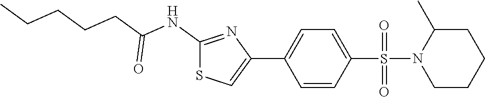 [SMILES: CCCCCC(=O)Nc1nc(-c2ccc(S(=O)(=O)N3CCCCC3C)cc2)cs1] |
| 1876 | 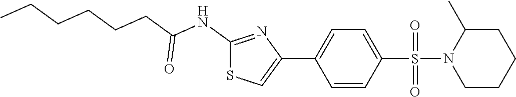 [SMILES: CCCCCCC(=O)Nc1nc(-c2ccc(S(=O)(=O)N3CCCCC3C)cc2)cs1] |
| 1877 | 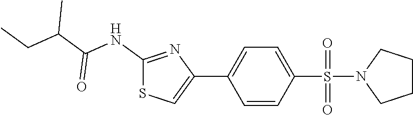 [SMILES: CCC(C)C(=O)Nc1nc(-c2ccc(S(=O)(=O)N3CCCC3)cc2)cs1] |
| 1878 | 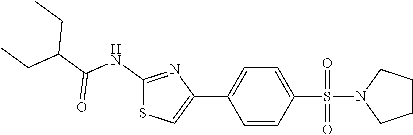 [SMILES: CCC(CC)C(=O)Nc1nc(-c2ccc(S(=O)(=O)N3CCCC3)cc2)cs1] |
| 1879 | 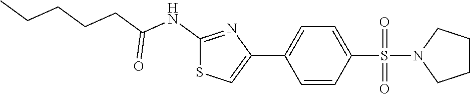 [SMILES: CCCCCC(=O)Nc1nc(-c2ccc(S(=O)(=O)N3CCCC3)cc2)cs1] |
| 1880 | 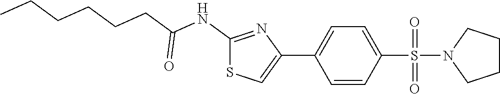 [SMILES: CCCCCCC(=O)Nc1nc(-c2ccc(S(=O)(=O)N3CCCC3)cc2)cs1] |
| 1881 | 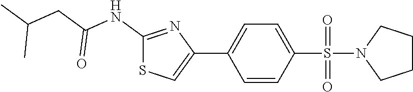 [SMILES: CC(C)CC(=O)Nc1nc(-c2ccc(S(=O)(=O)N3CCCC3)cc2)cs1] |
| 1882 | 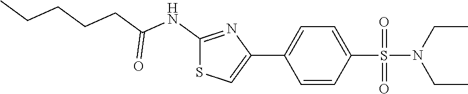 [SMILES: CCCCCC(=O)Nc1nc(-c2ccc(S(=O)(=O)N(CC)CC)cc2)cs1] |
| 1883 | 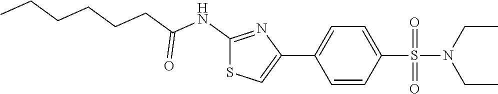 [SMILES: CCCCCCC(=O)Nc1nc(-c2ccc(S(=O)(=O)N(CC)CC)cc2)cs1] |
| 1884 | 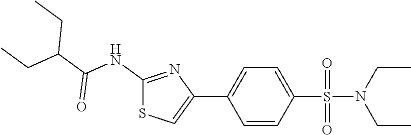 [SMILES: CCC(CC)C(=O)Nc1nc(-c2ccc(S(=O)(=O)N(CC)CC)cc2)cs1] |
| 1885 | 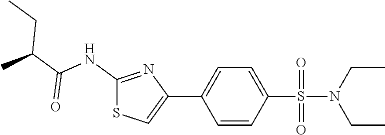 [SMILES: CC[C@H](C)C(=O)Nc1nc(-c2ccc(S(=O)(=O)N(CC)CC)cc2)cs1] |
| 1886 | 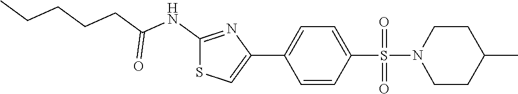 [SMILES: CCCCCC(=O)Nc1nc(-c2ccc(S(=O)(=O)N3CCC(C)CC3)cc2)cs1] |
| 1887 | 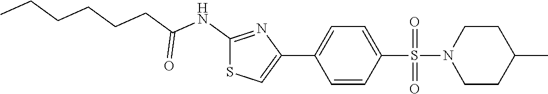 [SMILES: CCCCCCC(=O)Nc1nc(-c2ccc(S(=O)(=O)N3CCC(C)CC3)cc2)cs1] |
| 1888 | 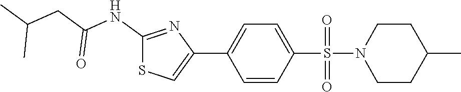 [SMILES: CC(C)CC(=O)Nc1nc(-c2ccc(S(=O)(=O)N3CCC(C)CC3)cc2)cs1] |
| 1889 | 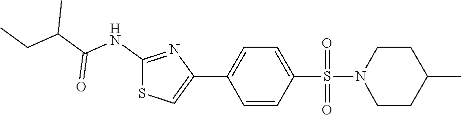 [SMILES: CCC(C)C(=O)Nc1nc(-c2ccc(S(=O)(=O)N3CCC(C)CC3)cc2)cs1] |
| 1890 | 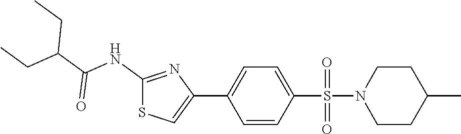 [SMILES: CCC(CC)C(=O)Nc1nc(-c2ccc(S(=O)(=O)N3CCC(C)CC3)cc2)cs1] |
| 1891 | 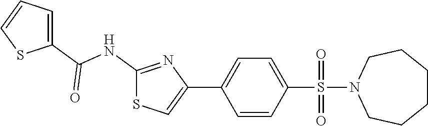 [SMILES: O=C(Nc1nc(-c2ccc(S(=O)(=O)N3CCCCCC3)cc2)cs1)c1cccs1] |
| 1892 | 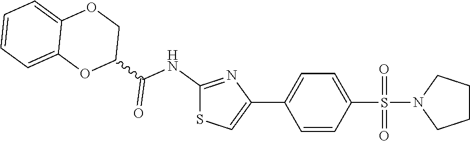 [SMILES: O=C(Nc1nc(-c2ccc(S(=O)(=O)N3CCCC3)cc2)cs1)C1COc2ccccc2O1] |
| 1893 | 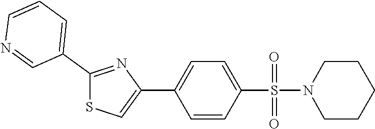 [SMILES: O=S(=O)(c1ccc(-c2csc(-c3cccnc3)n2)cc1)N1CCCCC1] |
| 1894 | 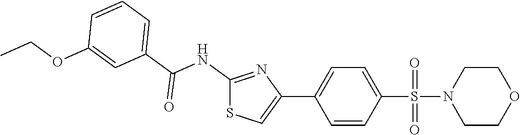 [SMILES: CCOc1cccc(C(=O)Nc2nc(-c3ccc(S(=O)(=O)N4CCOCC4)cc3)cs2)c1] |
| 1895 | 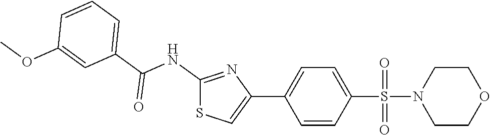 [SMILES: COc1cccc(C(=O)Nc2nc(-c3ccc(S(=O)(=O)N4CCOCC4)cc3)cs2)c1] |
| 1896 | 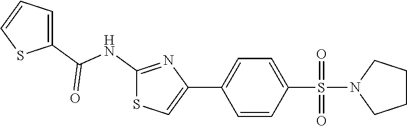 [SMILES: O=C(Nc1nc(-c2ccc(S(=O)(=O)N3CCCC3)cc2)cs1)c1cccs1] |
| 1897 | 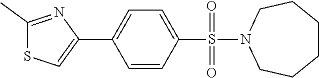 [SMILES: Cc1nc(-c2ccc(S(=O)(=O)N3CCCCCC3)cc2)cs1] |
| 1898 | 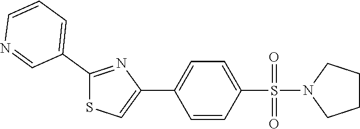 [SMILES: O=S(=O)(c1ccc(-c2csc(-c3cccnc3)n2)cc1)N1CCCC1] |
| 1899 | 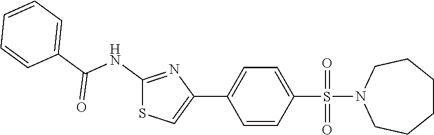 [SMILES: O=C(Nc1nc(-c2ccc(S(=O)(=O)N3CCCCCC3)cc2)cs1)c1ccccc1] |
| 1900 | 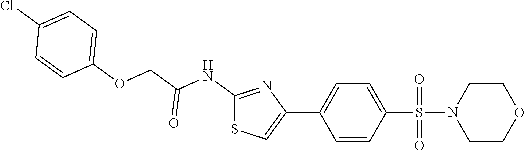 [SMILES: O=C(COc1ccc(Cl)cc1)Nc1nc(-c2ccc(S(=O)(=O)N3CCOCC3)cc2)cs1] |
| 1901 | 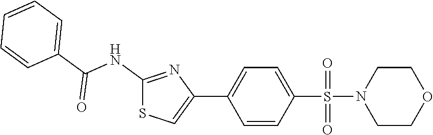 [SMILES: O=C(Nc1nc(-c2ccc(S(=O)(=O)N3CCOCC3)cc2)cs1)c1ccccc1] |
| 1902 | 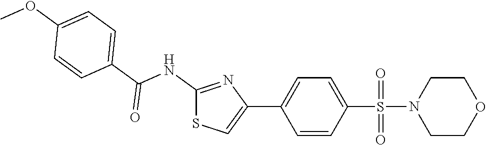 [SMILES: COc1ccc(C(=O)Nc2nc(-c3ccc(S(=O)(=O)N4CCOCC4)cc3)cs2)cc1] |
| 1903 | 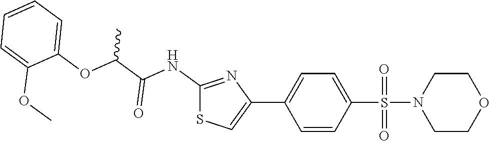 [SMILES: COc1ccccc1OC(C)C(=O)Nc1nc(-c2ccc(S(=O)(=O)N3CCOCC3)cc2)cs1] |
| 1904 | 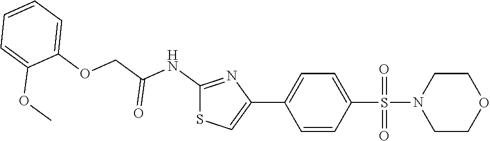 [SMILES: COc1ccccc1OCC(=O)Nc1nc(-c2ccc(S(=O)(=O)N3CCOCC3)cc2)cs1] |
| 1905 | 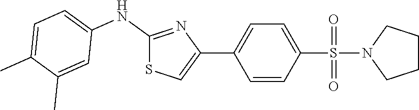 [SMILES: Cc1ccc(Nc2nc(-c3ccc(S(=O)(=O)N4CCCC4)cc3)cs2)cc1C] |
| 1906 | 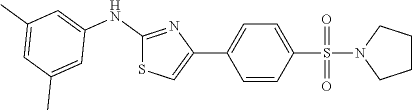 [SMILES: Cc1cc(C)cc(Nc2nc(-c3ccc(S(=O)(=O)N4CCCC4)cc3)cs2)c1] |
| 1907 | 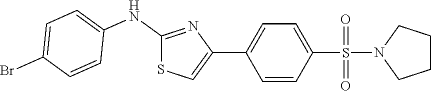 [SMILES: O=S(=O)(c1ccc(-c2csc(Nc3ccc(Br)cc3)n2)cc1)N1CCCC1] |
| 1908 | 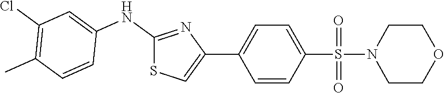 [SMILES: Cc1ccc(Nc2nc(-c3ccc(S(=O)(=O)N4CCOCC4)cc3)cs2)cc1Cl] |
| 1909 | 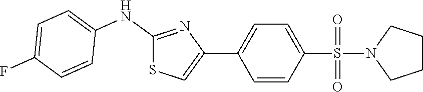 [SMILES: O=S(=O)(c1ccc(-c2csc(Nc3ccc(F)cc3)n2)cc1)N1CCCC1] |
| 1910 | 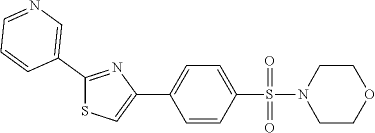 [SMILES: O=S(=O)(c1ccc(-c2csc(-c3cccnc3)n2)cc1)N1CCOCC1] |
| 1911 | 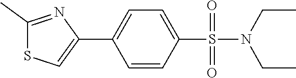 [SMILES: CCN(CC)S(=O)(=O)c1ccc(-c2csc(C)n2)cc1] |
| 1912 | 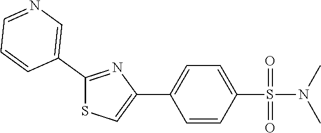 [SMILES: CN(C)S(=O)(=O)c1ccc(-c2csc(-c3cccnc3)n2)cc1] |
| 1913 | 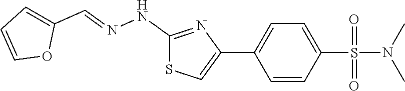 [SMILES: CN(C)S(=O)(=O)c1ccc(-c2csc(N/N=C/c3ccco3)n2)cc1] |
| 1914 | 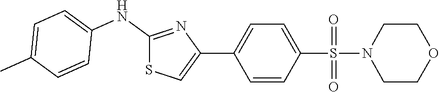 [SMILES: Cc1ccc(Nc2nc(-c3ccc(S(=O)(=O)N4CCOCC4)cc3)cs2)cc1] |
| 1915 | 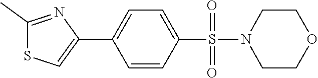 [SMILES: Cc1nc(-c2ccc(S(=O)(=O)N3CCOCC3)cc2)cs1] |
| 1916 | 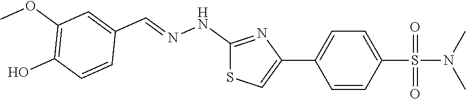 |
| 1917 | 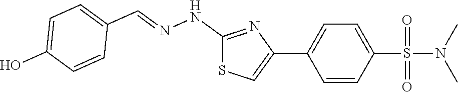 [SMILES: CN(C)S(=O)(=O)c1ccc(-c2csc(N/N=C/c3ccc(O)cc3)n2)cc1] |
| 1918 | 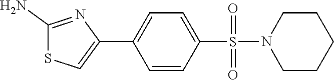 [SMILES: Nc1nc(-c2ccc(S(=O)(=O)N3CCCCC3)cc2)cs1] |
| 1919 | 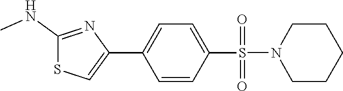 [SMILES: CNc1nc(-c2ccc(S(=O)(=O)N3CCCCC3)cc2)cs1] |
| 1920 | 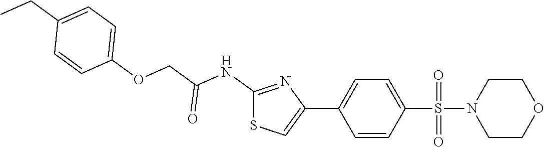 [SMILES: CCc1ccc(OCC(=O)Nc2nc(-c3ccc(S(=O)(=O)N4CCOCC4)cc3)cs2)cc1] |
| 1921 | 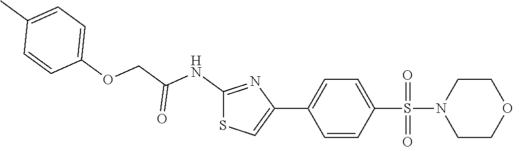 [SMILES: Cc1ccc(OCC(=O)Nc2nc(-c3ccc(S(=O)(=O)N4CCOCC4)cc3)cs2)cc1] |
| 1922 | 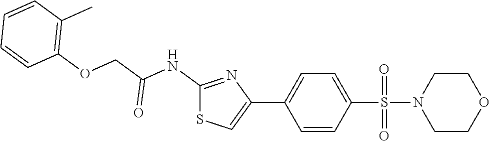 [SMILES: Cc1ccccc1OCC(=O)Nc1nc(-c2ccc(S(=O)(=O)N3CCOCC3)cc2)cs1] |
| 1923 | 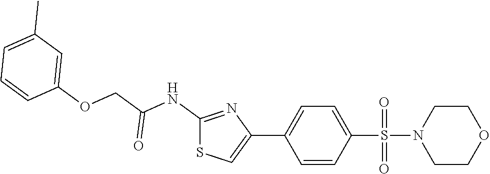 [SMILES: Cc1cccc(OCC(=O)Nc2nc(-c3ccc(S(=O)(=O)N4CCOCC4)cc3)cs2)c1] |
| 1924 | 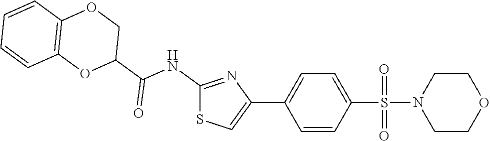 [SMILES: O=C(Nc1nc(-c2ccc(S(=O)(=O)N3CCOCC3)cc2)cs1)C1COc2ccccc2O1] |
| 1925 | 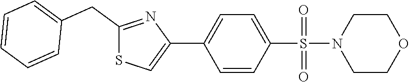 [SMILES: O=S(=O)(c1ccc(-c2csc(Cc3ccccc3)n2)cc1)N1CCOCC1] |
| 1926 | 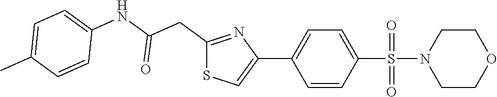 [SMILES: Cc1ccc(NC(=O)Cc2nc(-c3ccc(S(=O)(=O)N4CCOCC4)cc3)cs2)cc1] |
| 1927 | 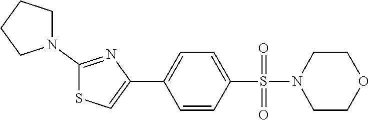 [SMILES: O=S(=O)(c1ccc(-c2csc(N3CCCC3)n2)cc1)N1CCOCC1] |
| 1928 | 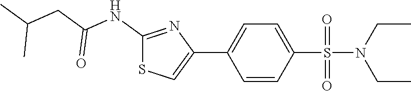 [SMILES: CCN(CC)S(=O)(=O)c1ccc(-c2csc(NC(=O)CC(C)C)n2)cc1] |
| 1929 | 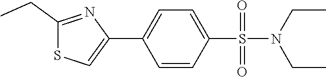 [SMILES: CCc1nc(-c2ccc(S(=O)(=O)N(CC)CC)cc2)cs1] |
| 1930 | 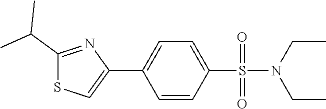 [SMILES: CCN(CC)S(=O)(=O)c1ccc(-c2csc(C(C)C)n2)cc1] |
| 1931 | 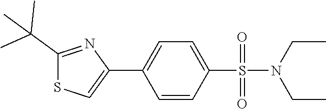 [SMILES: CCN(CC)S(=O)(=O)c1ccc(-c2csc(C(C)(C)C)n2)cc1] |
| 1932 | 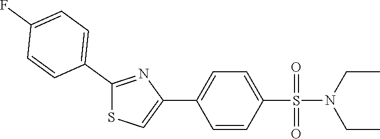 [SMILES: CCN(CC)S(=O)(=O)c1ccc(-c2csc(-c3ccc(F)cc3)n2)cc1] |
| 1933 | 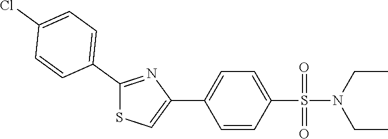 [SMILES: CCN(CC)S(=O)(=O)c1ccc(-c2csc(-c3ccc(Cl)cc3)n2)cc1] |
| 1934 | 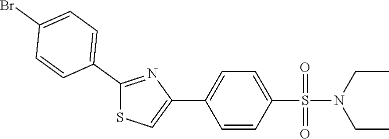 [SMILES: CCN(CC)S(=O)(=O)c1ccc(-c2csc(-c3ccc(Br)cc3)n2)cc1] |
| 1935 | 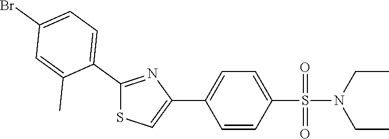 [SMILES: CCN(CC)S(=O)(=O)c1ccc(-c2csc(-c3ccc(Br)cc3C)n2)cc1] |
| 1936 | 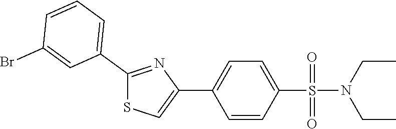 [SMILES: CCN(CC)S(=O)(=O)c1ccc(-c2csc(-c3cccc(Br)c3)n2)cc1] |
| 1937 | 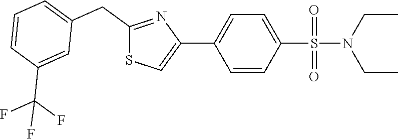 [SMILES: CCN(CC)S(=O)(=O)c1ccc(-c2csc(Cc3cccc(C(F)(F)F)c3)n2)cc1] |
| 1938 | 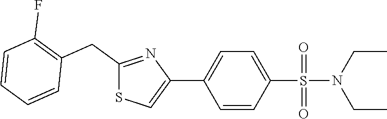 [SMILES: CCN(CC)S(=O)(=O)c1ccc(-c2csc(Cc3ccccc3F)n2)cc1] |
| 1939 | 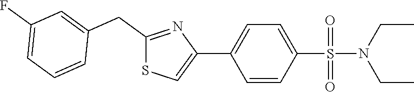 [SMILES: CCN(CC)S(=O)(=O)c1ccc(-c2csc(Cc3cccc(F)c3)n2)cc1] |
| 1940 | 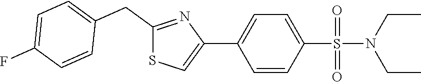 [SMILES: CCN(CC)S(=O)(=O)c1ccc(-c2csc(Cc3ccc(F)cc3)n2)cc1] |
| 1941 | 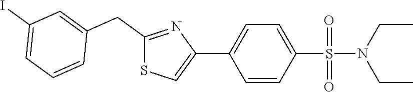 [SMILES: CCN(CC)S(=O)(=O)c1ccc(-c2csc(Cc3cccc(I)c3)n2)cc1] |
| 1942 | 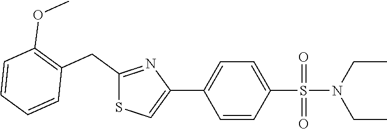 [SMILES: CCN(CC)S(=O)(=O)c1ccc(-c2csc(Cc3ccccc3OC)n2)cc1] |
| 1943 | 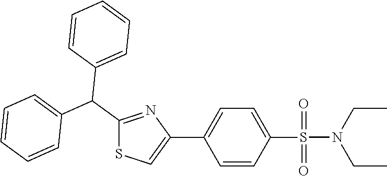 [SMILES: CCN(CC)S(=O)(=O)c1ccc(-c2csc(C(c3ccccc3)c3ccccc3)n2)cc1] |
| 1944 | 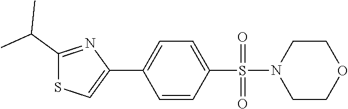 [SMILES: CC(C)c1nc(-c2ccc(S(=O)(=O)N3CCOCC3)cc2)cs1] |
| 1945 | 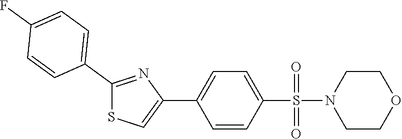 [SMILES: O=S(=O)(c1ccc(-c2csc(-c3ccc(F)cc3)n2)cc1)N1CCOCC1] |

In some embodiments, the compound has the structure of Formula (XXXVIII):

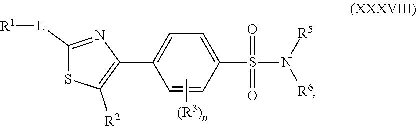

wherein: R¹ is substituted or unsubstituted C₃-C₈cycloalkyl; L is absent, C₁-C₄alkylene, —NR⁴—, —CH═N—NR⁴—, —NR⁴C(═O)—, or —C(═O)NR⁴—,—C(═O)NR⁴(C₁-C₄alkylene)-, —NR⁴C(═O)(C₁-C₄alkylene)-, —(C₁-C₄alkylene)C(═O) NR⁴—, —(C₁-C₄alkylene)NR⁴C(═O)—, —C(═O)NR⁴(C₁-C₄alkylene)O—, —NR⁴C(═O)(C₁-C₄alkylene)O—, —O(C₁-C₄alkylene)C(═O)NR⁴—, or —O(C₁-C₄alkylene) NR⁴C(═O)—; R² is hydrogen, halogen, —CN, —OH, substituted or unsubstituted C₁-C₆alkyl, substituted or unsubstituted C₁-C₆haloalkyl, substituted or unsubstituted C₁-C₆alkoxy, or substituted or unsubstituted C₁-C₆haloalkoxy; each R³ is independently selected from the group consisting of hydrogen, halogen, —CN, —OH, substituted or unsubstituted C₁-C₆ alkyl, substituted or unsubstituted C₁-C₆haloalkyl, substituted or unsubstituted C₁-C₆alkoxy, and substituted or unsubstituted C₁-C₆haloalkoxy; n is 0, 1, 2, 3, or 4; R⁴ is hydrogen, C₁-C₆alkyl, C₁-C₆ haloalkyl, C₃-C₈cycloalkyl, or substituted or unsubstituted aryl; R⁵ and R⁶ are each independently selected from the group consisting of hydrogen, substituted or unsubstituted C₁-C₆alkyl, substituted or unsubstituted C₁-C₆haloalkyl, substituted or unsubstituted C₃-C₈cycloalkyl, substituted or unsubstituted C₂-C₈heterocycloalkyl, substituted or unsubstituted aryl, substituted or unsubstituted heteroaryl, substituted or unsubstituted —C₁-C₂alkylene(aryl), and substituted or unsubstituted —C₁-C₂alkylene (heteroaryl); or R⁵ and R⁶ are taken together with the nitrogen to which they are attached form a substituted or unsubstituted 4-, 5-, 6-, or 7-membered heterocycloalkyl.

In some embodiments, L is —C(═O)NR⁴—; R² is hydrogen; R³ is hydrogen; R⁴ is hydrogen; and n is 0. In some embodiments, R¹ is cyclopropyl, cyclobutyl, cyclopentyl, or cyclohexyl. In some embodiments, R⁵ and R⁶ are each independently substituted or unsubstituted C₁-C₆alkyl. In some embodiments, R⁵ and R⁶ are each methyl or ethyl. In some embodiments, R⁵ and R⁶ are taken together with the nitrogen to which they are attached form a substituted or unsubstituted 4-, 5-, 6-, or 7-membered heterocycloalkyl. In some embodiments, R⁵ and R⁶ are taken together with the nitrogen to which they are attached form a pyrrolidinyl, morpholinyl, piperidinyl, 4-methylpiperidinyl, 2-methylpiperidinyl, 3-methylpiperidinyl, thiomorpholinyl, piperazinyl, or 4-methylpiperazinyl.

In some embodiments, the compound is one of the following:

| | Compound |
|---|---|
| 1946 | 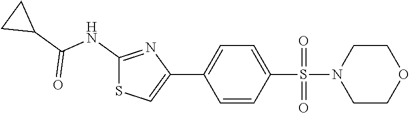 [SMILES: O=C(Nc1nc(-c2ccc(S(=O)(=O)N3CCOCC3)cc2)cs1)C1CC1] |
| 1947 | 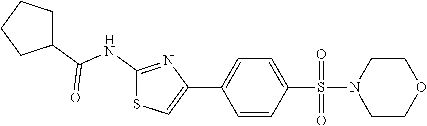 [SMILES: O=C(Nc1nc(-c2ccc(S(=O)(=O)N3CCOCC3)cc2)cs1)C1CCCC1] |
| 1948 | 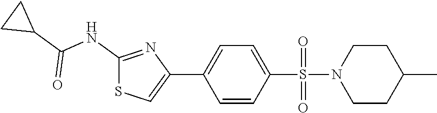 [SMILES: CC1CCN(S(=O)(=O)c2ccc(-c3csc(NC(=O)C4CC4)n3)cc2)CC1] |
| 1949 | 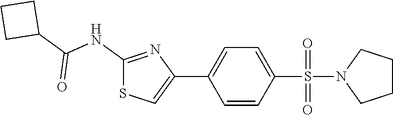 [SMILES: O=C(Nc1nc(-c2ccc(S(=O)(=O)N3CCCC3)cc2)cs1)C1CCC1] |
| 1950 | 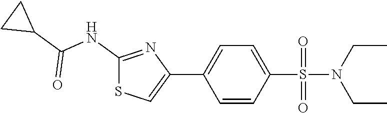 [SMILES: CCN(CC)S(=O)(=O)c1ccc(-c2csc(NC(=O)C3CC3)n2)cc1] |
| 1951 | 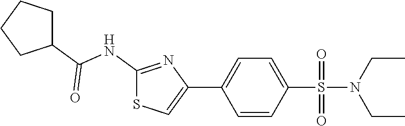 [SMILES: CCN(CC)S(=O)(=O)c1ccc(-c2csc(NC(=O)C3CCCC3)n2)cc1] |
| 1952 | 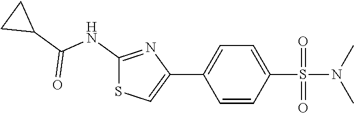 [SMILES: CN(C)S(=O)(=O)c1ccc(-c2csc(NC(=O)C3CC3)n2)cc1] |
| 1953 | 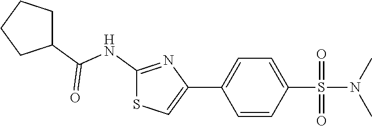 [SMILES: CN(C)S(=O)(=O)c1ccc(-c2csc(NC(=O)C3CCCC3)n2)cc1] |
| 1954 | 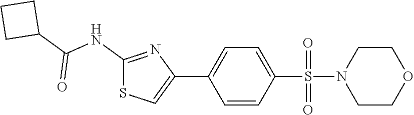 [SMILES: O=C(Nc1nc(-c2ccc(S(=O)(=O)N3CCOCC3)cc2)cs1)C1CCC1] |
| 1955 | 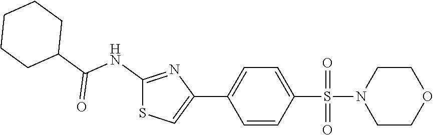 [SMILES: O=C(Nc1nc(-c2ccc(S(=O)(=O)N3CCOCC3)cc2)cs1)C1CCCCC1] |
| 1956 | 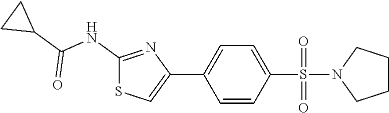 [SMILES: O=C(Nc1nc(-c2ccc(S(=O)(=O)N3CCCC3)cc2)cs1)C1CC1] |
| 1957 | 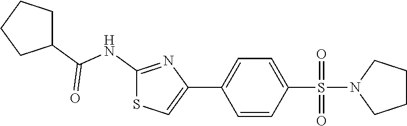 [SMILES: O=C(Nc1nc(-c2ccc(S(=O)(=O)N3CCCC3)cc2)cs1)C1CCCC1] |
| 1958 | 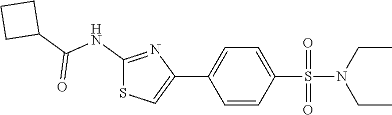 [SMILES: CCN(CC)S(=O)(=O)c1ccc(-c2csc(NC(=O)C3CCC3)n2)cc1] |
| 1959 | 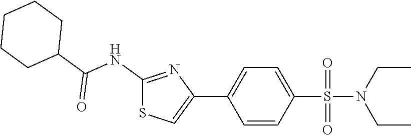 [SMILES: CCN(CC)S(=O)(=O)c1ccc(-c2csc(NC(=O)C3CCCCC3)n2)cc1] |
| 1960 | 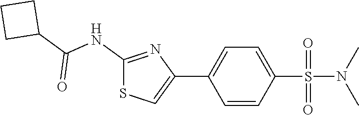 [SMILES: CN(C)S(=O)(=O)c1ccc(-c2csc(NC(=O)C3CCC3)n2)cc1] |

In some embodiments, the compound has the structure of Formula (XXXIX):

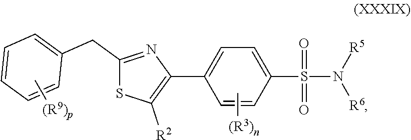

wherein: R² is hydrogen, halogen, —CN, —OH, substituted or unsubstituted C₁-C₆alkyl, substituted or unsubstituted C₁-C₆haloalkyl, or substituted or unsubstituted C₁-C₆alkoxy; each R³ is independently selected from the group consisting of hydrogen, halogen, —CN, —OH, substituted or unsubstituted C₁-C₆ alkyl, substituted or unsubstituted C₁-C₆haloalkyl, and substituted or unsubstituted C₁-C₆alkoxy; R⁵ and R⁶ are each independently selected from the group consisting of hydrogen, substituted or unsubstituted C₁-C₆alkyl, substituted or unsubstituted C₁-C₆haloalkyl, substituted or unsubstituted C₃-C₈cycloalkyl, substituted or unsubstituted C₂-C₈heterocycloalkyl, substituted or unsubstituted aryl, substituted or unsubstituted heteroaryl, substituted or unsubstituted —C₁-C₂alkylene(aryl), and substituted or unsubstituted —C₁-C₂alkylene(heteroaryl); or R⁵ and R⁶ are taken together with the nitrogen to which they are attached form a substituted or unsubstituted 4-, 5-, 6-, or 7-membered heterocycloalkyl; each R⁹ is independently selected from the group consisting of hydrogen, halogen, —CN, —OH, substituted or unsubstituted C₁-C₆alkyl, C₁-C₆haloalkyl, substituted or unsubstituted C₁-C₆alkoxy, and substituted or unsubstituted C₁-C₆haloalkoxy; n is 0, 1, 2, 3, or 4; and p is 1, 2, 3, 4, or 5; or a pharmaceutically acceptable salt or solvate thereof.

In some embodiments, R² is hydrogen; R³ is hydrogen; and n is 0. In some embodiments, R⁹ is halogen, C₁-C₆alkyl, C₁-C₆haloalkyl, or C₁-C₆alkoxy. In some embodiments, R⁵ and R⁶ are each independently substituted or unsubstituted C₁-C₆alkyl. In some embodiments, R⁵ and R⁶ are each methyl or ethyl. In some embodiments, R⁵ and R⁶ are taken together with the nitrogen to which they are attached form a substituted or unsubstituted 4-, 5-, 6-, or 7-membered heterocycloalkyl. In some embodiments, R⁵ and R⁶ are taken together with the nitrogen to which they are attached form a pyrrolidinyl, morpholinyl, piperidinyl, 4-methylpiperidinyl, 2-methylpiperidinyl, 3-methylpiperidinyl, thiomorpholinyl, piperazinyl, or 4-methylpiperazinyl. In some embodiments, each R⁹ is independently selected from the group consisting of halogen, —CN, —OH, substituted or unsubstituted C₁-C₆alkyl, C₁-C₆haloalkyl, substituted or unsubstituted C₁-C₆alkoxy, and substituted or unsubstituted C₁-C₆haloalkoxy.

In some embodiments, the compound is one of the following:

| | Compound |
|---|---|
| 1961 | 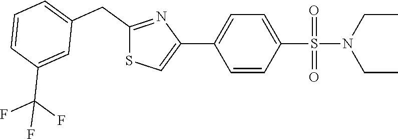 [SMILES: CCN(CC)S(=O)(=O)c1ccc(-c2csc(Cc3cccc(C(F)(F)F)c3)n2)cc1] |
| 1962 | 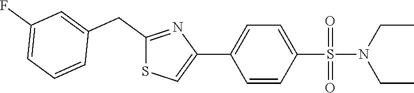 [SMILES: CCN(CC)S(=O)(=O)c1ccc(-c2csc(Cc3cccc(F)c3)n2)cc1] |
| 1963 | 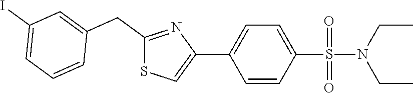 [SMILES: CCN(CC)S(=O)(=O)c1ccc(-c2csc(Cc3cccc(I)c3)n2)cc1] |
| 1964 | 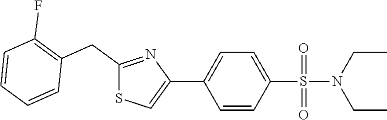 [SMILES: CCN(CC)S(=O)(=O)c1ccc(-c2csc(Cc3ccccc3F)n2)cc1] |
| 1965 | 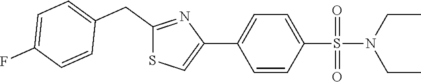 [SMILES: CCN(CC)S(=O)(=O)c1ccc(-c2csc(Cc3ccc(F)cc3)n2)cc1] |
| 1966 | 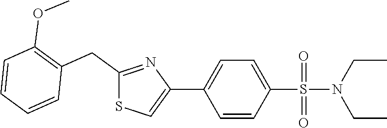 [SMILES: CCN(CC)S(=O)(=O)c1ccc(-c2csc(Cc3ccccc3OC)n2)cc1] |

In some embodiments, the compound has the structure of Formula (XL):

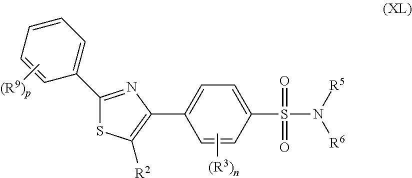

wherein: R is hydrogen, halogen, —CN, —OH, substituted or unsubstituted C₁-C₆alkyl, substituted or unsubstituted C₁-C₆haloalkyl, substituted or unsubstituted C₁-C₆alkoxy, or substituted or unsubstituted C₁-C₆haloalkoxy; each R³ is independently selected from the group consisting of hydrogen, halogen, —CN, —OH, substituted or unsubstituted C₁-C₆alkyl, substituted or unsubstituted C₁-C₆haloalkyl, substituted or unsubstituted C₁-C₆alkoxy, and substituted or unsubstituted C₁-C₆haloalkoxy; R⁵ and R⁶ are each independently selected from the group consisting of hydrogen, substituted or unsubstituted C₁-C₆alkyl, substituted or unsubstituted C₁-C₆haloalkyl, substituted or unsubstituted C₃-C₈cycloalkyl, substituted or unsubstituted C₂-C₈heterocycloalkyl, substituted or unsubstituted aryl, substituted or unsubstituted heteroaryl, substituted or unsubstituted —C₁-C₂alkylene(aryl), and substituted or unsubstituted —C₁-C₂ alkylene(heteroaryl); or R⁵ and R⁶ are taken together with the nitrogen to which they are attached form a substituted or unsubstituted 4-, 5-, 6-, or 7-membered heterocycloalkyl; each R⁹ is independently selected from the group consisting of hydrogen, halogen, —CN, —OH, substituted or unsubstituted C₁-C₆alkyl, C₁-C₆ haloalkyl, substituted or unsubstituted C₁-C₆alkoxy, and substituted or unsubstituted C₁-C₆haloalkoxy; n is 0, 1, 2, 3, or 4; and p is 0, 1, 2, 3, 4, or 5; or a pharmaceutically acceptable salt, or solvate thereof.

In some embodiments, R³ is hydrogen; R⁴ is hydrogen; and n is 0. In some embodiments, each R is independently halogen or substituted or unsubstituted C₁-C₆alkyl; and p is 1 or 2. In some embodiments, R⁵ and R⁶ are each independently substituted or unsubstituted C₁-C₆alkyl. In some embodiments, R⁵ and R⁶ are each methyl or ethyl. In some embodiments, R⁵ and R⁶ are taken together with the nitrogen to which they are attached form a substituted or unsubstituted 4-, 5-, 6-, or 7-membered heterocycloalkyl. In some embodiments, R⁵ and R⁶ are taken together with the nitrogen to which they are attached form a pyrrolidinyl, morpholinyl, piperidinyl, 4-methylpiperidinyl, 2-methylpiperidinyl, 3-methylpiperidinyl, thiomorpholinyl, piperazinyl, or 4-methylpiperazinyl.

In some embodiments, the compound is one of the following:

| | Compound |
|---|---|
| 1967 | 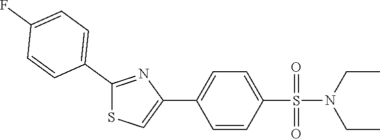 [SMILES: CCN(CC)S(=O)(=O)c1ccc(-c2csc(-c3ccc(F)cc3)n2)cc1] |
| 1968 | 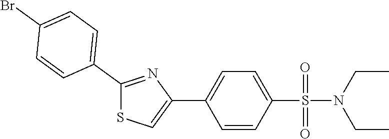 [SMILES: CCN(CC)S(=O)(=O)c1ccc(-c2csc(-c3ccc(Br)cc3)n2)cc1] |
| 1969 | 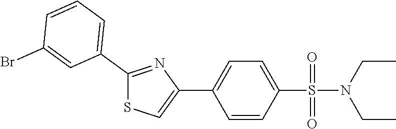 [SMILES: CCN(CC)S(=O)(=O)c1ccc(-c2csc(-c3cccc(Br)c3)n2)cc1] |
| 1970 | 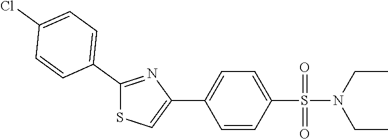 [SMILES: CCN(CC)S(=O)(=O)c1ccc(-c2csc(-c3ccc(Cl)cc3)n2)cc1] |
| 1971 | 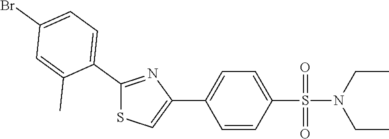 [SMILES: CCN(CC)S(=O)(=O)c1ccc(-c2csc(-c3ccc(Br)cc3C)n2)cc1] |
| 1972 | 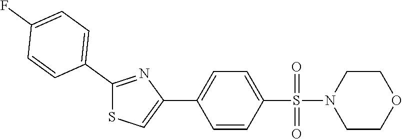 [SMILES: O=S(=O)(c1ccc(-c2csc(-c3ccc(F)cc3)n2)cc1)N1CCOCC1] |

In some embodiments, the compound has the structure of Formula (XLI):

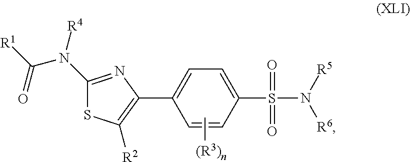

wherein: R¹ is substituted or unsubstituted C₁-C₈alkyl, substituted or unsubstituted C₁-C₈haloalkyl, substituted or unsubstituted C₃-C₈cycloalkyl, substituted or unsubstituted C₂-C₈heterocycloalkyl, substituted or unsubstituted aryl, substituted or unsubstituted heteroaryl, substituted or unsubstituted —C₁-C₂alkylene(aryl), or substituted or unsubstituted —C₁-C₂alkylene(heteroaryl); R is hydrogen, halogen, —CN, —OH, substituted or unsubstituted C₁-C₆alkyl, substituted or unsubstituted C₁-C₆haloalkyl, substituted or unsubstituted C₁-C₆alkoxy, or substituted or unsubstituted C₁-C₆haloalkoxy; each R³ is independently selected from the group consisting of hydrogen, halogen, —CN, —OH, substituted or unsubstituted C₁-C₆ alkyl, substituted or unsubstituted C₁-C₆haloalkyl, substituted or unsubstituted C₁-C₆alkoxy, and substituted or unsubstituted C₁-C₆haloalkoxy; R⁴ is hydrogen, C₁-C₆alkyl, C₁-C₆haloalkyl, C₃-C₈cycloalkyl, or substituted or unsubstituted aryl; R⁵ and R⁶ are each independently selected from the group consisting of methyl or ethyl; or R⁵ and R⁶ are taken together with the nitrogen to which they are attached form a piperidinyl, 4-methylpiperidinyl, 2-methylpiperidinyl, 3-methylpiperidinyl, piperazinyl, or 4-methylpiperazinyl; and n is 0, 1, 2, 3, or 4; or a pharmaceutically acceptable salt or solvate thereof.

In some embodiments, R² is hydrogen; R³ is hydrogen; R⁴ is hydrogen; and n is 0. In some embodiments, R¹ is substituted or unsubstituted C₁-C₈alkyl. In some embodiments, R¹ is methyl, ethyl, n-propyl, i-propyl, n-butyl, sec-butyl, i-butyl, t-butyl, 1-ethyl-propyl, n-pentyl, n-hexyl, or n-heptyl. In some embodiments, R¹ is 1-ethyl-propyl or sec-butyl. In some embodiments, R⁵ and R⁶ are each methyl or ethyl. In some embodiments, R⁵ and R⁶ are taken together with the nitrogen to which they are attached form a 4-methylpiperidinyl or 2-methylpiperidinyl.

In some embodiments, the compound is one of the following:

| | Compound |
|---|---|
| 1973 | 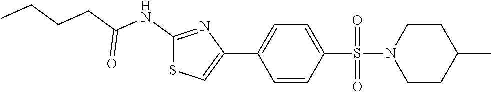 [SMILES: CCCCC(=O)Nc1nc(-c2ccc(S(=O)(=O)N3CCC(C)CC3)cc2)cs1] |
| 1974 | 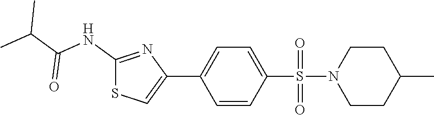 [SMILES: CC1CCN(S(=O)(=O)c2ccc(-c3csc(NC(=O)C(C)C)n3)cc2)CC1] |
| 1975 | 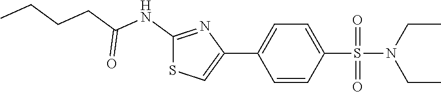 [SMILES: CCCCC(=O)Nc1nc(-c2ccc(S(=O)(=O)N(CC)CC)cc2)cs1] |
| 1976 | 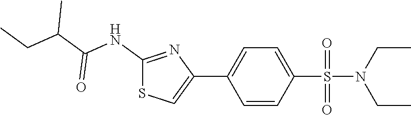 [SMILES: CCC(C)C(=O)Nc1nc(-c2ccc(S(=O)(=O)N(CC)CC)cc2)cs1] |
| 1977 | 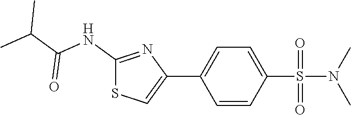 [SMILES: CC(C)C(=O)Nc1nc(-c2ccc(S(=O)(=O)N(C)C)cc2)cs1] |
| 1978 | 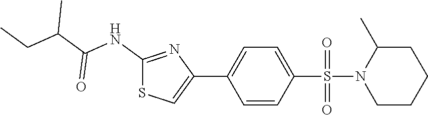 [SMILES: CCC(C)C(=O)Nc1nc(-c2ccc(S(=O)(=O)N3CCCCC3C)cc2)cs1] |
| 1979 | 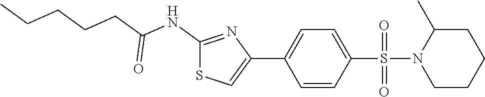 [SMILES: CCCCCC(=O)Nc1nc(-c2ccc(S(=O)(=O)N3CCCCC3C)cc2)cs1] |
| 1980 | 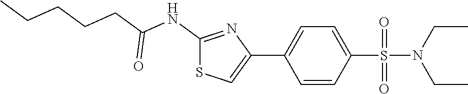 [SMILES: CCCCCC(=O)Nc1nc(-c2ccc(S(=O)(=O)N(CC)CC)cc2)cs1] |
| 1981 | 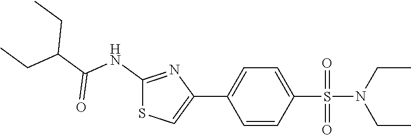 [SMILES: CCC(CC)C(=O)Nc1nc(-c2ccc(S(=O)(=O)N(CC)CC)cc2)cs1] |
| 1982 | 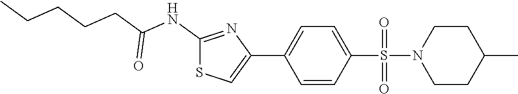 [SMILES: CCCCCC(=O)Nc1nc(-c2ccc(S(=O)(=O)N3CCC(C)CC3)cc2)cs1] |
| 1983 | 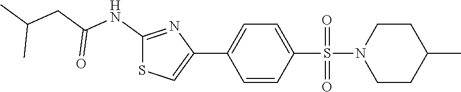 [SMILES: CC(C)CC(=O)Nc1nc(-c2ccc(S(=O)(=O)N3CCC(C)CC3)cc2)cs1] |
| 1984 | 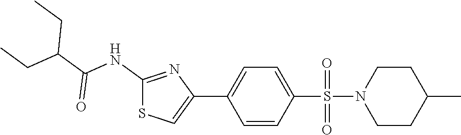 [SMILES: CCC(CC)C(=O)Nc1nc(-c2ccc(S(=O)(=O)N3CCC(C)CC3)cc2)cs1] |
| 1985 | 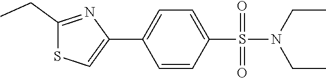 [SMILES: CCc1nc(-c2ccc(S(=O)(=O)N(CC)CC)cc2)cs1] |
| 1986 | 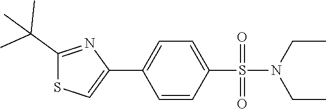 [SMILES: CCN(CC)S(=O)(=O)c1ccc(-c2csc(C(C)(C)C)n2)cc1] |
| 1987 | 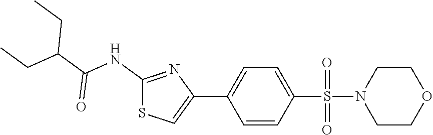 [SMILES: CCC(CC)C(=O)Nc1nc(-c2ccc(S(=O)(=O)N3CCOCC3)cc2)cs1] |
| 1988 | 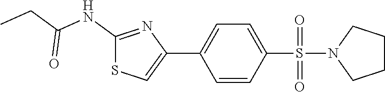 [SMILES: CCC(=O)Nc1nc(-c2ccc(S(=O)(=O)N3CCCC3)cc2)cs1] |
| 1989 | 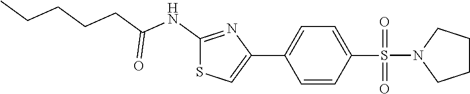 [SMILES: CCCCCC(=O)Nc1nc(-c2ccc(S(=O)(=O)N3CCCC3)cc2)cs1] |
| 1990 | 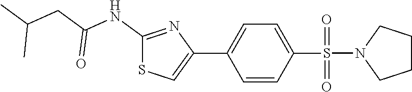 [SMILES: CC(C)CC(=O)Nc1nc(-c2ccc(S(=O)(=O)N3CCCC3)cc2)cs1] |
| 1991 | 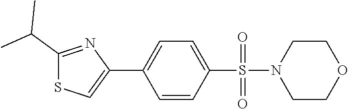 [SMILES: CC(C)c1nc(-c2ccc(S(=O)(=O)N3CCOCC3)cc2)cs1] |
| 1992 | 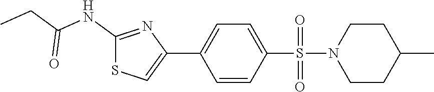 [SMILES: CCC(=O)Nc1nc(-c2ccc(S(=O)(=O)N3CCC(C)CC3)cc2)cs1] |
| 1993 | 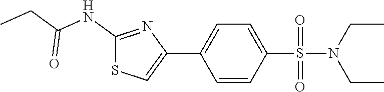 [SMILES: CCC(=O)Nc1nc(-c2ccc(S(=O)(=O)N(CC)CC)cc2)cs1] |
| 1994 | 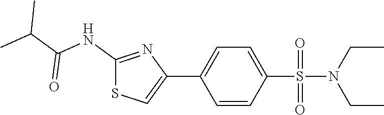 [SMILES: CCN(CC)S(=O)(=O)c1ccc(-c2csc(NC(=O)C(C)C)n2)cc1] |
| 1995 | 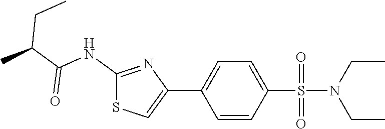 [SMILES: CC[C@H](C)C(=O)Nc1nc(-c2ccc(S(=O)(=O)N(CC)CC)cc2)cs1] |
| 1996 | 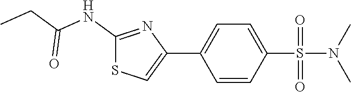 [SMILES: CCC(=O)Nc1nc(-c2ccc(S(=O)(=O)N(C)C)cc2)cs1] |
| 1997 | 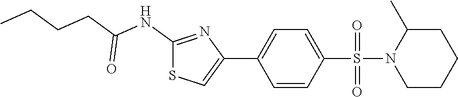 [SMILES: CCCCC(=O)Nc1nc(-c2ccc(S(=O)(=O)N3CCCCC3C)cc2)cs1] |
| 1998 | 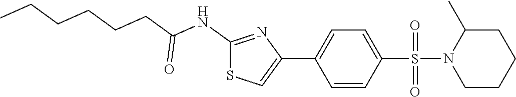 [SMILES: CCCCCCC(=O)Nc1nc(-c2ccc(S(=O)(=O)N3CCCCC3C)cc2)cs1] |
| 1999 | 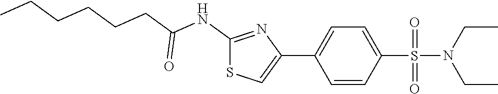 [SMILES: CCCCCCC(=O)Nc1nc(-c2ccc(S(=O)(=O)N(CC)CC)cc2)cs1] |
| 2000 | 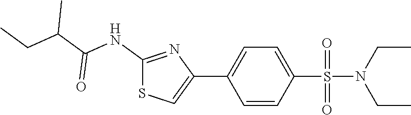 [SMILES: CCC(C)C(=O)Nc1nc(-c2ccc(S(=O)(=O)N(CC)CC)cc2)cs1] |
| 2001 | 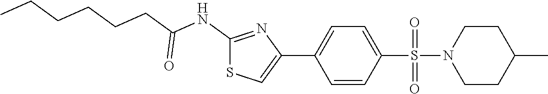 [SMILES: CCCCCCC(=O)Nc1nc(-c2ccc(S(=O)(=O)N3CCC(C)CC3)cc2)cs1] |
| 2002 | 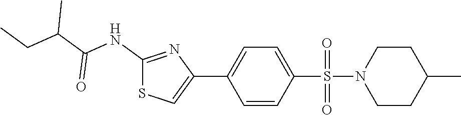 [SMILES: CCC(C)C(=O)Nc1nc(-c2ccc(S(=O)(=O)N3CCC(C)CC3)cc2)cs1] |
| 2003 | 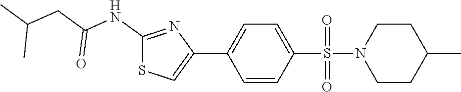 [SMILES: CC(C)CC(=O)Nc1nc(-c2ccc(S(=O)(=O)N3CCC(C)CC3)cc2)cs1] |
| 2004 | 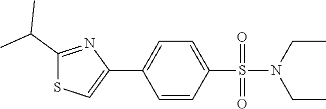 [SMILES: CCN(CC)S(=O)(=O)c1ccc(-c2csc(C(C)C)n2)cc1] |
| 2005 | 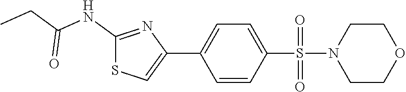 [SMILES: CCC(=O)Nc1nc(-c2ccc(S(=O)(=O)N3CCOCC3)cc2)cs1] |
| 2006 | 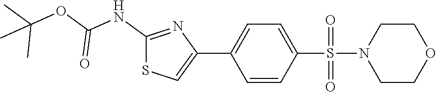 [SMILES: CC(C)(C)OC(=O)Nc1nc(-c2ccc(S(=O)(=O)N3CCOCC3)cc2)cs1] |
| 2007 | 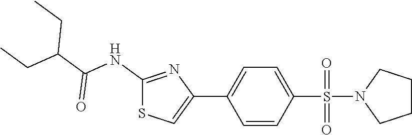 [SMILES: CCC(CC)C(=O)Nc1nc(-c2ccc(S(=O)(=O)N3CCCC3)cc2)cs1] |
| 2008 | 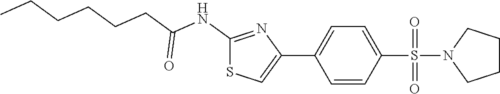 [SMILES: CCCCCCC(=O)Nc1nc(-c2ccc(S(=O)(=O)N3CCCC3)cc2)cs1] |
| 2009 | 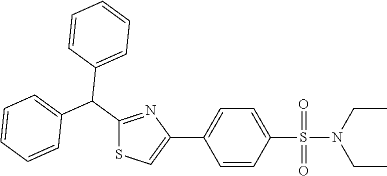 [SMILES: CCN(CC)S(=O)(=O)c1ccc(-c2csc(C(c3ccccc3)c3ccccc3)n2)cc1] |

In some embodiments, the compound has the structure of

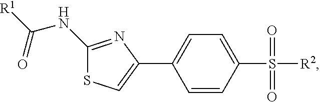

wherein R¹ and R² are as follows:

| R¹ | R² |
|---|---|
| n-butyl | morpholine |
| PhOCH₂ | morpholine |
| 2-OMePh | morpholine |
| i-butyl | morpholine |
| methyl | morpholine |
| 2-furan | morpholine |
| methyl | thiomorpholine |
| sec-butyl | morpholine |
| cyclopropyl | morpholine |
| cyclopentyl | morpholine |
| O-t-butyl | morpholine |
| Ethyl | 4-methylpiperidyl |
| cyclopropyl | 4-methylpiperidyl |
| i-propyl | pyrrolidine |
| cyclobutyl | pyrrolidine |
| Ethyl | N(Et)₂ |
| i-propyl | N(Et)₂ |
| cyclobutyl | N(Et)₂ |
| sec-butyl | N(Et)₂ |
| i-propyl | N(Me)₂ |
| cyclopropyl | N(Me)₂ |
| cyclopentyl | N(Me)₂ |
| n-butyl | 2-methylpiperidyl |
| n-heptyl | 2-methylpiperidyl |
| 1-Et-propyl | pyrrolidine |
| n-hexyl | pyrrolidine |
| n-pentyl | N(Et)₂ |
| 1-Et-propyl | N(Et)₂ |
| n-pentyl | 4-methylpiperidyl |
| i-butyl | 4-methylpiperidyl |
| 1-Et-propyl | 4-methylpiperidyl |
| 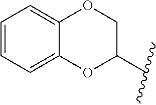 [SMILES: *C1COc2ccccc2O1] | pyrrolidine |
| 3-methyoxyphenyl | morpholine |
| phenyl | azepane |
| phenyl | morpholine |
| 1-(2-methoxy | morpholine |
| phenoxy)ethyl | |
| 4-ethylphenoxy | morpholine |
| methyl | |
| 2-methylphenoxy | morpholine |
| methyl | |
| 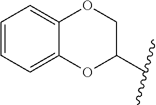 [SMILES: *C1COc2ccccc2O1] | morpholine |
| n-pentyl | 2-methylpiperidyl |
| 3ClPhOCH₂ | morpholine |
| 2-furan | pyrrolidine |
| i-propyl | morpholine |
| n-butyl | pyrrolidine |
| 2-thiophene | morpholine |
| n-butyl | thiomorpholine |
| ethyl | morpholine |
| 1-Et-propyl | morpholine |
| cyclobutyl | morpholine |
| cyclohexyl | morpholine |
| N-butyl | 4-methylpiperidyl |
| i-propyl | 4-methylpiperidyl |
| ethyl | pyrrolidine |
| cyclopropyl | pyrrolidine |
| cyclopentyl | pyrrolidine |
| n-butyl | N(Et)₂ |
| cyclopropyl | N(Et)₂ |
| cyclopentyl | N(Et)₂ |
| cyclohexyl | N(Et)₂ |
| ethyl | N(Me)₂ |
| cyclobutyl | N(Me)₂ |
| sec-butyl | 2-methylpiperidyl |
| n-propyl | 2-methylpiperidyl |
| sec-butyl | pyrrolidine |
| n-pentyl | pyrrolidine |
| i-butyl | pyrrolidine |
| n-hexyl | N(Et)₂ |
| (S) sec-butyl | N(Et)₂ |
| n-hexyl | 4-methylpiperidyl |
| sec-butyl | 4-methylpiperidyl |
| thien-2-yl | Azepane |
| 3-ethoxyphenyl | morpholine |
| thien-2-yl | pyrrolidine |
| 4-chlorophenoxy | morpholine |
| methyl | |
| 4-methoxyphenyl | morpholine |
| 2-methoxy | morpholine |
| phenoxymethyl | |
| 4-methylphenoxy | morpholine |
| methyl | |
| 3-methylphenoxy | morpholine |
| methyl | |
| 2-methylpropyl | N-Et)₂ |
| n-hexyl | 2-methylpiperidyl |

In some embodiments, the compound has the structure of

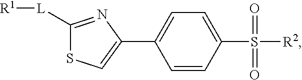

wherein R¹ and R² are as follows:

| R¹—L— | R² |
|---|---|
| Pyridin-3-yl | piperidine |
| Pyridin-3-yl | pyrrolidine |
| 3,5-dimethylphenyl | pyrrolidine |
| amino | |
| 3-chloro-4-methyl | morpholine |
| phenylamino | |
| Pyridin-3-yl | morpholine |
| Pyridin-3-yl | N(Me)₂ |
| 4-methylphenylamino | morpholine |
| 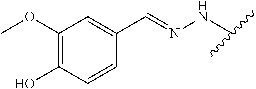 [SMILES: *N/N=C/c1ccc(O)c(OC)c1] | N(Me)₂ |
| amino | piperidine |
| benzyl | morpholine |
| pyrrolidine | morpholine |
| isopropyl | N(Et)₂ |
| 4-fluorophenyl | N(Et)₂ |
| 4-bromophenyl | N(Et)₂ |
| 3-bromophenyl | N(Et)₂ |
| 2-fluorophenylmethyl | N(Et)₂ |
| 4-fluorophenylmethyl | N(Et)₂ |
| 2-methoxyphenyl | N(Et)₂ |
| methyl | |
| isopropyl | morpholine |
| methyl | azepane |
| 3,4-dimethylphenyl | pyrrolidine |
| amino | |
| 4-bromophenylamino | pyrrolidine |
| 4-fluorophenylamino | pyrrolidine |
| methyl | N(Et)₂ |
| 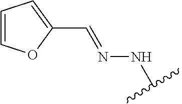 [SMILES: *N/N=C/c1ccco1] | N(Me)₂ |
| methyl | morpholine |
| 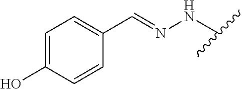 [SMILES: *N/N=C/c1ccc(O)cc1] | N(Me)₂ |
| methylamino | piperidine |
| 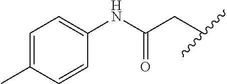 [SMILES: *CC(=O)Nc1ccc(C)cc1] | morpholine |
| ethyl | N(Et)₂ |
| tert-butyl | N(Et)₂ |
| 4-chlorophenyl | N(Et)₂ |
| 4-bromo-2-methyl | N(Et)₂ |
| phenyl | |
| 3-trifluoromethyl | N(Et)₂ |
| phenylmethyl | |
| 3-fluorophenylmethyl | N(Et)₂ |
| 3-iodophenylmethyl | N(Et)₂ |
| 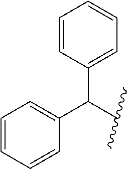 [SMILES: *C(c1ccccc1)c1ccccc1] | N(Et)₂ |
| 4-fluorophenyl | morpholine |

In some embodiments, the compound has the structure of Formula (XLII):

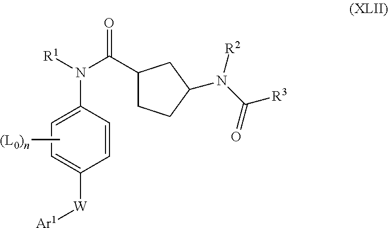

wherein Ar¹ is a phenyl ring or a 5- or 6-membered monocyclic heteroaryl-group which has 1 to 4 heteroatoms independently selected from the group consisting of N, O and S; and wherein said phenyl ring or said 5- or 6-membered monocyclic heteroaryl-group may be linked to a group Ar² via a single bond or may be condensed to a group Ar², wherein one or more C-atoms may be substituted independently of one another with a substituent L1; and wherein one or more imino-groups may be substituted independently of one another with a substituent R^{N0}; and Ar² is a 5- or 6-membered saturated or unsaturated carbocyclic ring which may have 1 or 2 heteroatoms independently selected from the group consisting of N, O and S, or may have 3 or 4 N-atoms; and W is a single bond, —C≡C—, —CH═CH—, —CH₂—CH₂— or —CH₂—O—; R¹ is C₁₋₄-alkyl; R² is H or C₁₋₄-alkyl; R³ is C₁₋₆-alkyl, C₃₋₆-alkenyl, C₃₋₆-alkynyl, C₃₋₆-cycloalkyl or R^{N1}R^{N2}N—, wherein each of said alkyl, alkenyl, alkynyl and cycloalkyl groups may be substituted with one or more substituents selected from the group consisting of R^{N1}R^{N2}N—, C₁₋₄-alkyl-O—C(═O)—R^{N0}N—, HO—, C₁₋₄-alkyloxy, C₃₋₇-cycloalkyl, phenyl and pyridinyl, wherein said cycloalkyl, phenyl and pyridinyl may be substituted with one or more substituents L2; R^{N0} is H or C₁₋₄-alkyl; R^{N1}, R^{N2} independently of each other selected from H, C₁₋₄-alkyl, phenyl, pyridinyl, phenyl-C₁₋₃-alkyl, pyridinyl-C₁₋₃-alkyl or R^{N1}, R^{N2} are linked to each other to form with the N-atom of the R^{N1}R^{N2}N— group a heterocyclic ring selected from the group consisting of pyrrolidinyl, piperidinyl, morpholinyl, thiomorpholinyl, piperazinyl or 4-(C₁₋₄-alkyl)-piperazinyl; L0, L₁ independently of each other is selected from the group consisting of F, Cl, Br, cyano, OH, C₁₋₄-alkyl, C₂₋₄-alkenyl, C₁₋₄-alkyloxy, C₁₋₄-alkylcarbonyl, R^{N1}R^{N2}N—, R^{N1}R^{N2}N—C₁₋₃₋alkyl-, R^{N1}R^{N2}N—CO—, C₁₋₄-alkyl-CO—NR^{N0}— and C₁₋₄-alkyl-SO₂— NR^{N0}—, wherein alkyl-groups may be mono- or polyfluorinated; L2 is selected from the group consisting of F, Cl, Br, cyano, OH, C₁₋₄-alkyl, C₁₋₄-alkyloxy, R^{N1}R^{N2}N—, R^{N1}R^{N2}N—C₁₋₃-alkyl-, wherein alkyl-groups may be mono- or polyfluorinated; n is an integer from 0 to 4; while, unless otherwise stated, the above-mentioned alkyl groups may be straight-chain or branched, and the tautomers, the stereoisomers thereof, the mixtures thereof and the salts thereof. In some embodiments, the compound has the structure of Formula (XLII-RS), (XLII-RR), (XLII-SS), (XLII-SR):

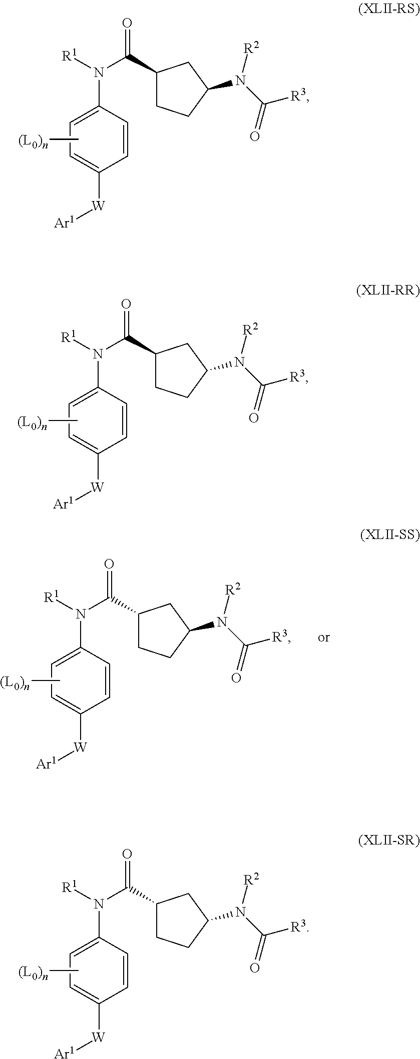

According to one aspect the invention refers to a mixture of compounds of the formula XLII-RS and XLII-SR. The mixture may be a racemic mixture. Preferably the mixture comprises more than 50% by weight of compounds of the formula XLII-RS. Even more preferably the mixture comprises more than 80% by weight of compounds of the formula XLII-RS. According to another aspect the invention refers to a mixture of compounds of the formula XLII-RR and XLII-SS.

Unless otherwise stated, the groups, residues, and substituents, particularly Ar¹, Ar², W, R¹, R², R³, R^{N0}, R^{N1}, R^{N2}, L0, L1, L2 and the index n are defined as above and hereinafter. If residues, substituents, or groups occur several times in a compound, as for example L0, L1 or L2, they may have the same or different meanings. Some preferred meanings of individual groups and substituents of the compounds according to the invention will be given hereinafter.

In some embodiments, Ar¹ preferably is phenyl, thienyl, pyridinyl, pyrrolyl, imidazolyl, triazolyl, furanyl or oxazolyl. In some embodiments, Ar¹ even more preferably is phenyl, thienyl or pyridinyl. In some embodiments, Ar¹ preferably is phenyl, thienyl, pyridinyl, pyrrolyl, imidazolyl, triazolyl, furanyl, isoxazolyl or oxazolyl, all of which are condensed to a group Ar². In some embodiments, Ar¹ preferably is phenyl, thienyl, pyridinyl, pyrrolyl, imidazolyl, triazolyl, furanyl, isoxazolyl or oxazolyl, all of which are linked to a group Ar² via a single bond. In some embodiments, Ar² preferably is phenyl, pyridyl, pyrrolyl, dihydropyrrolyl, furanyl, dihydrofuranyl or dioxolyl. In some embodiments, Ar¹ even more preferably is benzooxazole, benzoimidazole, benzotriazole, benzofuran, 2,3-dihydrobenzofuran, benzo[1,3]dioxole, naphthyl, quinoline or isoquinoline. In some embodiments, Ar¹ most preferably is

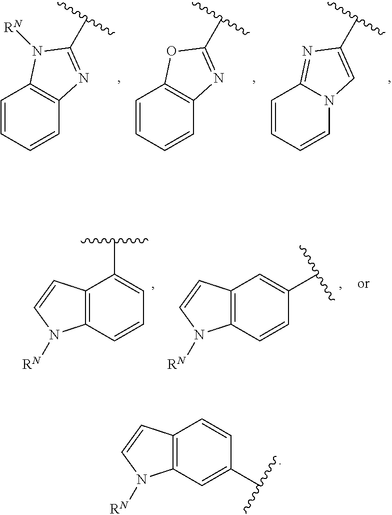

In some embodiments, Ar¹ even more preferably is biphenyl, phenylpyridinyl or pyridinylphenyl; for example 5-phenyl-pyridin-2-yl. In the hereinbefore mentioned embodiments the group Ar¹, including any group Ar², one or more C-atoms may be substituted independently of one another with a substituent L1; and one or more imino-groups may be substituted independently of one another with a substituent R^{N0}.

In some embodiments, L0 is preferably independently of each other selected from the group consisting of F, C₁, Br, cyano, OH, C₁₋₃-alkyl, C₂₋₄-alkenyl, C₁₋₃-alkyloxy, C₁₋₄-alkylcarbonyl, amino, C₁₋₃-alkylamino, and di-(C₁₋₃-alkyl)amino, wherein alkyl-groups may be mono- or polyfluorinated. Preferred examples of the substituent L0 are F, Cl, Br, cyano, OH, methyl, difluoromethyl, trifluoromethyl, ethyl, propyl, i-propyl, ethenyl, propenyl, methoxy, difluoromethoxy, trifluoromethoxy, ethoxy, propoxy, i-propoxy, methylcarbonyl, ethylcarbonyl, amino, methylamino, and dimethylamino. In some embodiments, L1 is preferably independently of each other selected from the group consisting of F, Cl, Br, cyano, OH, C₁₋₃-alkyl, C₂₋₄-alkenyl, C₁₋₃-alkyloxy, C₁₋₄-alkylcarbonyl, amino, C₁₋₃-alkylamino, di-(C₁₋₃-alkyl)amino, aminocarbonyl, di-C₁₋₃-alkylaminocarbonyl, di-(C₁₋₃-alkyl)aminocarbonyl, C₁₋₃-alkyl-carbonylamino, and C₁₋₃-alkyl-sulfonylamino, wherein alkyl-groups may be mono- or polyfluorinated. Preferred examples of the substituent L1 are F, Cl, Br, cyano, OH, methyl, difluoromethyl, trifluoromethyl, ethyl, propyl, i-propyl, ethenyl, propenyl, methoxy, difluoromethoxy, trifluoromethoxy, ethoxy, propoxy, i-propoxy, methylcarbonyl, ethylcarbonyl, amino, methylamino, dimethylamino, aminocarbonyl, methylaminocarbonyl, dimethylaminocarbonyl, methylcarbonylamino, and methylsulfonylamino.

In some embodiments, n is 0, 1, 2 or 3, even more preferably 0, 1 or 2. In some embodiments, W is a single bond. In some embodiments, is —C≡C—. In some embodiments, W is —CH═CH—. In some embodiments, W is —CH₂—CH₂—. In some embodiments, W is —CH₂—O—. In some embodiments, R¹ preferably denotes methyl or ethyl, in particular methyl. In some embodiments, R² preferably denotes H or methyl, in particular H. In some embodiments, R³ preferably denotes C₁₋₆-alkyl, C₃₋₄-alkenyl, C₃₋₄-alkynyl or C₃₋₆-cycloalkyl or R^{N1}R^{N2}N—, wherein each of said alkyl, alkenyl, alkynyl and cycloalkyl groups may be substituted with one or more substituents selected from the group consisting of R^{N1}′ R^{N2}N—, C₁₋₄-alkyl-O—C(═O)—R^{N0}N—, HO—, C₁₋₄-alkyloxy, C₃₋₇-cycloalkyl, phenyl and pyridinyl, wherein said cycloalkyl, phenyl and pyridinyl may be substituted with one or more substituents L2. In some embodiments, R³ preferably denotes C₁₋₆-alkyl, C₃₋₆-cycloalkyl, C₁₋₄-alkyloxy-C₁₋₅-alkyl, R^{N1}R^{N2}N—, R^{N1}R^{N2}N—C₁₋₆-alkyl, wherein alkyl groups may be mono- or polyfluorinated. Examples of preferred substituents R³ are methyl, difluoromethyl, trifluoromethyl, ethyl, 1-methylethyl, propyl, cyclopropyl, methylamino, ethylamino, dimethylamino, diethylamino, aminopentyl, aminohexyl, dimethylaminopentyl, dimethylaminohexyl, 4-(dimethylaminomethyl)-cyclohexylmethyl and 3-(N-methylpiperazin-1-yl)-propyl.

In some embodiments, L2 is preferably independently of each other selected from the group consisting of F, Cl, Br, cyano, OH, C₁₋₃-alkyl, C₁₋₃-alkyloxy, C₁₋₄-alkylcarbonyl, amino, C₁₋₃-alkylamino, di-(C₁₋₃-alkyl)amino, amino-C₁₋₃-alkyl, C₁₋₃-alkylamino-C₁₋₃-alkyl, di-(C₁₋₃-alkyl)amino-C₁₋₃-alkyl, pyrrolidinyl-C₁₋₃-alkyl, piperidinyl-C₁₋₃-alkyl, piperazinyl-C₁₋₃-alkyl, N—(C₁₋₃-alkyl)piperazinyl-C₁₋₃-alkyl, wherein each alkyl-group may be mono- or polyfluorinated. Preferred examples of the substituent L2 are F, Cl, Br, cyano, OH, methyl, difluoromethyl, trifluoromethyl, ethyl, propyl, i-propyl, ethenyl, methoxy, difluoromethoxy, trifluoromethoxy, ethoxy, propoxy, i-propoxy, methylcarbonyl, ethylcarbonyl, amino, methylamino, dimethylamino, aminomethyl, methylaminomethyl, dimethylaminomethyl, piperazinylmethyl, N-methylpiperazinylethyl.

In some embodiments, R^{N0} preferably is H, methyl or ethyl, in particular H or methyl. In some embodiments, R^{N1}, R^{N2} independently of each other are preferably selected from H, C₁₋₃-alkyl, or R^{N1}, R^{N2}are linked to each other to form with the N-atom of the R^{N1}R^{N2}N— group a heterocyclic ring selected from the group consisting of pyrrolidinyl, piperidinyl, morpholinyl, thiomorpholinyl, piperazinyl or 4-(C₁₋₄-alkyl)-piperazinyl. Preferred examples of the substituents R^{N1}, R^{N2} are H, methyl, ethyl or R^{N1}, R^{N2} are linked to each other to form with the N-atom of the —NR^{N1}R^{N2} group a heterocyclic ring selected from the group consisting of pyrrolidinyl, piperidinyl, piperazinyl or 4-methyl-piperazinyl.

In some embodiments, the compound is (1R,3S)-3-Propionylamino-cyclopentanecarboxylic acid N-biphenyl-4-yl-N-methyl-amide, (1R,3S)-3-Acetylamino-cyclopentanecarboxylic acid N-(4-benzooxazol-2-yl-phenyl)-N-methyl-amide, or (1R,3S)-3-Propionylamino-cyclopentanecarboxylic acid N-(4-benzooxazol-2-yl-phenyl)-N-methyl-amide.

In some embodiments, the compound is one of the following:

| | Compound |
|---|---|
| 2010 | 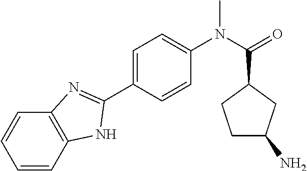 [SMILES: CN(C(=O)[C@@H]1CC[C@H](N)C1)c1ccc(-c2nc3ccccc3[nH]2)cc1] |
| 2011 | 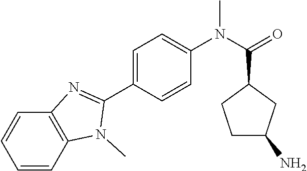 [SMILES: CN(C(=O)[C@@H]1CC[C@H](N)C1)c1ccc(-c2nc3ccccc3n2C)cc1] |
| 2012 | 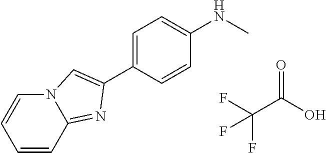 |
| 2013 | 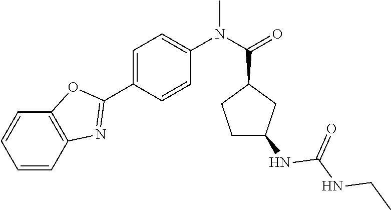 [SMILES: CCNC(=O)N[C@H]1CC[C@@H](C(=O)N(C)c2ccc(-c3nc4ccccc4o3)cc2)C1] |
| 2014 | 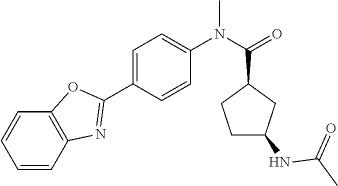 [SMILES: CC(=O)N[C@H]1CC[C@@H](C(=O)N(C)c2ccc(-c3nc4ccccc4o3)cc2)C1] |
| 2015 | 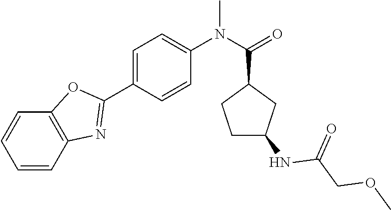 [SMILES: COCC(=O)N[C@H]1CC[C@@H](C(=O)N(C)c2ccc(-c3nc4ccccc4o3)cc2)C1] |
| 2016 | 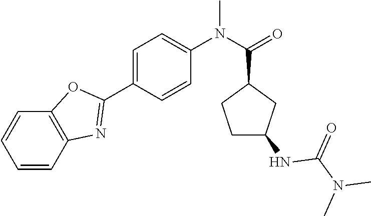 [SMILES: CN(C)C(=O)N[C@H]1CC[C@@H](C(=O)N(C)c2ccc(-c3nc4ccccc4o3)cc2)C1] |
| 2017 | 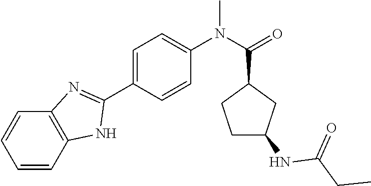 [SMILES: CCC(=O)N[C@H]1CC[C@@H](C(=O)N(C)c2ccc(-c3nc4ccccc4[nH]3)cc2)C1] |
| 2018 | 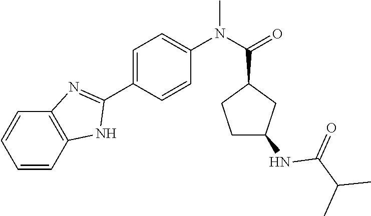 [SMILES: CC(C)C(=O)N[C@H]1CC[C@@H](C(=O)N(C)c2ccc(-c3nc4ccccc4[nH]3)cc2)C1] |
| 2019 | 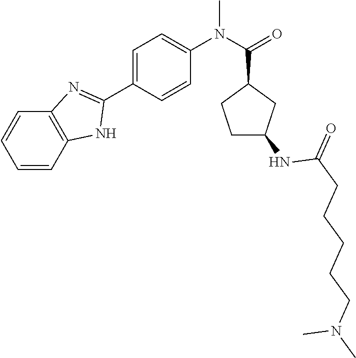 [SMILES: CN(C)CCCCCC(=O)N[C@H]1CC[C@@H](C(=O)N(C)c2ccc(-c3nc4ccccc4[nH]3)cc2)C1] |
| 2020 | 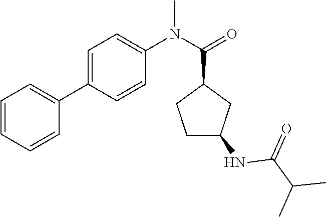 [SMILES: CC(C)C(=O)N[C@H]1CC[C@@H](C(=O)N(C)c2ccc(-c3ccccc3)cc2)C1] |
| 2021 | 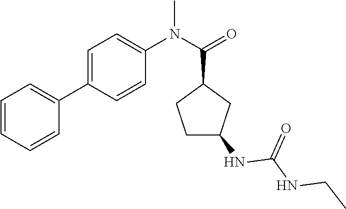 [SMILES: CCNC(=O)N[C@H]1CC[C@@H](C(=O)N(C)c2ccc(-c3ccccc3)cc2)C1] |
| 2022 | 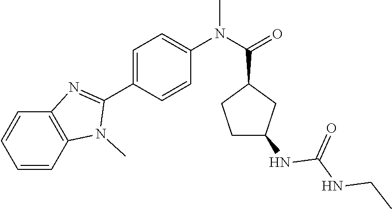 [SMILES: CCNC(=O)N[C@H]1CC[C@@H](C(=O)N(C)c2ccc(-c3nc4ccccc4n3C)cc2)C1] |
| 2023 | 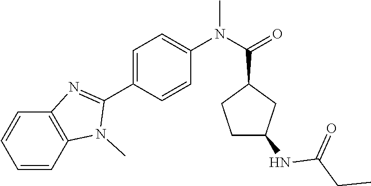 [SMILES: CCC(=O)N[C@H]1CC[C@@H](C(=O)N(C)c2ccc(-c3nc4ccccc4n3C)cc2)C1] |
| 2024 | 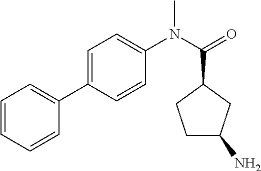 [SMILES: CN(C(=O)[C@@H]1CC[C@H](N)C1)c1ccc(-c2ccccc2)cc1] |
| 2025 | 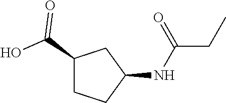 |
| 2026 | 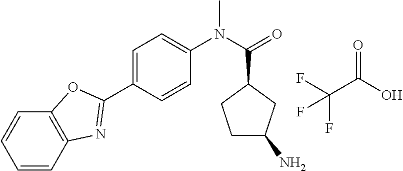 |
| 2027 | 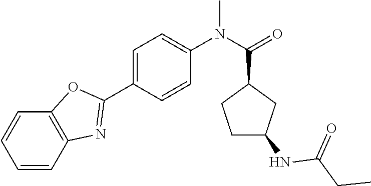 [SMILES: CCC(=O)N[C@H]1CC[C@@H](C(=O)N(C)c2ccc(-c3nc4ccccc4o3)cc2)C1] |
| 2028 | 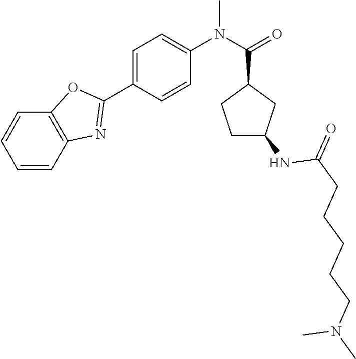 [SMILES: CN(C)CCCCCC(=O)N[C@H]1CC[C@@H](C(=O)N(C)c2ccc(-c3nc4ccccc4o3)cc2)C1] |
| 2029 | 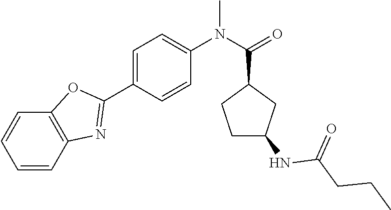 [SMILES: CCCC(=O)N[C@H]1CC[C@@H](C(=O)N(C)c2ccc(-c3nc4ccccc4o3)cc2)C1] |
| 2030 | 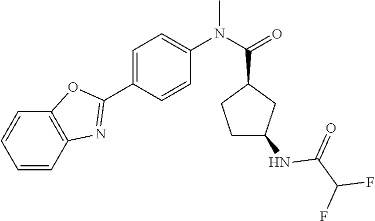 [SMILES: CN(C(=O)[C@@H]1CC[C@H](NC(=O)C(F)F)C1)c1ccc(-c2nc3ccccc3o2)cc1] |
| 2031 | 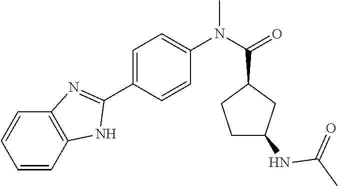 [SMILES: CC(=O)N[C@H]1CC[C@@H](C(=O)N(C)c2ccc(-c3nc4ccccc4[nH]3)cc2)C1] |
| 2032 | 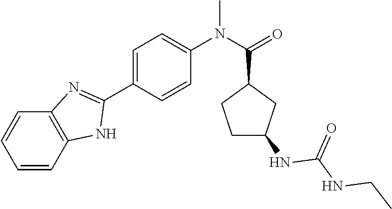 [SMILES: CCNC(=O)N[C@H]1CC[C@@H](C(=O)N(C)c2ccc(-c3nc4ccccc4[nH]3)cc2)C1] |
| 2033 | 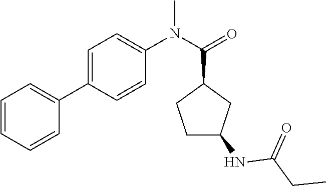 [SMILES: CCC(=O)N[C@H]1CC[C@@H](C(=O)N(C)c2ccc(-c3ccccc3)cc2)C1] |
| 2034 | 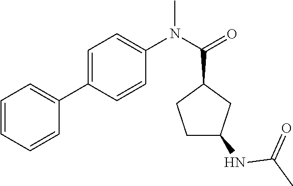 [SMILES: CC(=O)N[C@H]1CC[C@@H](C(=O)N(C)c2ccc(-c3ccccc3)cc2)C1] |
| 2035 | 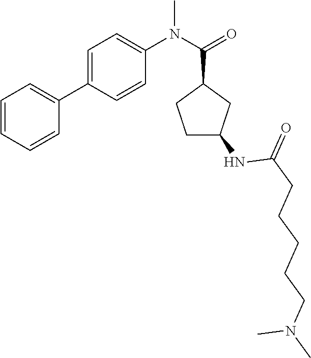 [SMILES: CN(C)CCCCCC(=O)N[C@H]1CC[C@@H](C(=O)N(C)c2ccc(-c3ccccc3)cc2)C1] |
| 2036 | 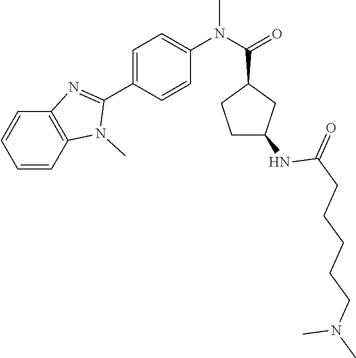 [SMILES: CN(C)CCCCCC(=O)N[C@H]1CC[C@@H](C(=O)N(C)c2ccc(-c3nc4ccccc4n3C)cc2)C1] |
| 2037 | 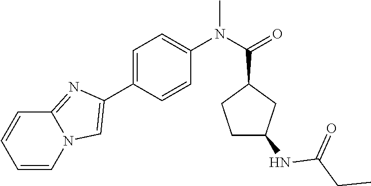 [SMILES: CCC(=O)N[C@H]1CC[C@@H](C(=O)N(C)c2ccc(-c3cn4ccccc4n3)cc2)C1] |

In some embodiments, the compound has the structure of Formula (XLIII):

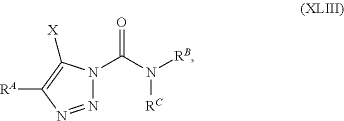

wherein: R^{A} is selected C₃₋₁₀ carbocyclyl, 3-14 membered heterocyclyl, C₆₋₁₄ aryl, 5-14 membered heteroaryl, and hydrogen; X is selected from hydrogen, —CN, —CHO, —C(═O)R^{X1}, —C(═O)NR^{X2}₂, —CO₂H, CO₂R^{X1}, —SO₂R^{X1}, —C(═NR^{X2})OR^{X1}, —C(═NR^{X2})NR^{X2}₂, —SO₂NR^{X2}₂, —SO₂R^{X1}, —SO₃H, —SO₂OR^{X1}, —SOR^{X1}, —C(═S)NR^{X2}₂, —C(═O)SR^{X1}, —C(═S)SR^{X1}, —P(═O)₂R^{X1}, —P(═O)(R^{X1})₂, —P(═O)₂NR^{X2}₂, —P(═O)(NR^{X2})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl; or R^{A} and X, together with the carbon atoms to which each is attached, are joined to form a 5-10 membered carbocyclyl, heterocyclyl, aryl or heteroaryl ring; R^{B} is selected from C₆₋₁₄ aryl, 5-14 membered heteroaryl, C₁₋₁₀ alkyl, C₂₋₁₀ alkenyl, C₂₋₁₀ alkynyl, 3-14 membered heteroaliphatic, C₃₋₁₀ carbocyclyl, and 3-14 membered heterocyclyl; R^{C} is selected from hydrogen, —OH, —OR^{C1}, —ONR^{C2}₂, —NR^{C2}₂, —C(═O)R^{C1}, —CHO, —CO₂R^{C1}, —C(═O)NR^{C2}₂, —C(═NR^{C2})OR^{C1}, C(═NR^{C2})NR^{C2}₂, —SO₂R^{C1}, —S(═O)R^{C1}, —Si(R^{C1})₃, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; or R^{B} and R^{C} together with the nitrogen (N) atom to which each is attached are joined to form a 5-14 membered carbocyclyl, heterocyclyl, aryl or heteroaryl ring; each R^{C1} and R^{X1} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each R^{C2} is, independently, selected from hydrogen, —OH, —OR^{C1}, —NR^{C3}₂, —CN, —C(═O)R^{C1}, C(═O)NR^{C3}₂, —CO₂R^{C1}, SO₂R^{C1}, —C(═NR^{C3})OR^{C1}, —C(═NR^{C3})NR^{C3}₂, —SO₂NR^{C3}₂, —SO₂R^{C3}, —SO₂OR^{C3}, —SOR^{C1}, —C(═S)NR^{C3}₂, —C(═O)SR^{C3}, —C(═S)SR^{C3}, —P(═O)₂R^{C1}, —P(═O)(R^{C1})₂, —P(═O)₂NR^{C3}₂, —P(═O)(NR^{C3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each R^{x2} is, independently, selected from hydrogen, —OH, —OR^{X1}, —NR^{X3}₂, —CN, —C(═O)R^{X1}, —C(═O)NR^{X3}₂, —CO₂R¹, —SO₂R^{X1}, —C(═NR^{X3})OR^{X1}, —C(═NR^{X3})NR^{X3}₂, —SO₂NR^{X3}₂, —SO₂R^{X3}, —SO₂OR^{X3}, —SOR^{X1}, —C(═S)NR^{X3}₂, —C(═O)SR^{X3}, —C(═S)SR^{X3}, —P(═O)₂R^{X1}, —P(═O)(R^{X1})₂, —P(═O)₂NR^{X3}₂, —P(═O)(NR^{X3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; and each R^{C3} and R^{X3} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl.

In some embodiments, R^{A} is selected from C₃₋₁₀ carbocyclyl, 3-14 membered heterocyclyl, C₆₋₁₄ aryl, 5-14 membered heteroaryl, and hydrogen; or R^{A} and X, together with the carbon atoms to which each is attached, are joined to form a 5-10 membered ring. In certain embodiments, R^{A} is selected from C₃₋₁₀ carbocyclyl, 3-14 membered heterocyclyl, C₆₋₁₄ aryl, 5-14 membered heteroaryl, and hydrogen. In certain embodiments, R^{A} is selected from C₆₋₁₄ aryl and 5-14 membered heteroaryl. In certain embodiments, R^{A} is C₃₋₁₀ carbocyclyl. Exemplary carbocyclyl groups include, but are not limited to, cyclopropyl (C₃), cyclobutyl (C₄), cyclopentyl (C₅), cyclopentenyl (C₅), cyclohexyl (C₆), cyclohexenyl (C₆), cyclohexadienyl (C₆), cycloheptyl (C₇), cycloheptadienyl (C₇), cycloheptatrienyl (C₇) and cyclooctyl (C₈). In certain embodiments, R^{A} is 3-14 membered heterocyclyl. Exemplary heterocyclyl groups include, but are not limited to, azirdinyl, oxiranyl, thiorenyl, azetidinyl, oxetanyl, thietanyl, tetrahydrofuranyl, dihydrofuranyl, tetrahydrothiophenyl, dihydrothiophenyl, pyrrolidinyl, dihydropyrrolyl, dioxolanyl, oxathiolanyl and dithiolanyl, piperidinyl, tetrahydropyranyl, dihydropyridinyl, thianyl, piperazinyl, morpholinyl, dithianyl, dioxanyl, azepanyl, oxepanyl thiepanyl, azocanyl, oxecanyl and thiocanyl. In certain embodiments, R^{A} is C₆₋₁₄ aryl. Exemplary aryl groups include, but are not limited to, phenyl, naphthyl and anthracyl. In certain embodiments, R^{A} is phenyl (C₆ aryl). In certain embodiments, R^{A} is naphthyl (C₁₀ aryl). In certain embodiments, R^{A} is 5-14 membered heteroaryl. In certain embodiments, R^{A} is 5-10 membered heteroaryl. In certain embodiments, R^{A} is 5-6 membered heteroaryl. In certain embodiments, R^{A} is 5,6-bicyclic heteroaryl. In certain embodiments, R^{A} is 6,6-bicyclic heteroaryl. In certain embodiments, R^{A} is a 5-membered heteroaryl group. Exemplary 5-membered heteroaryl groups include, but are not limited to, pyrrolyl, furanyl, thiophenyl, imidazolyl, pyrazolyl, oxazolyl, isoxazolyl, thiazolyl, isothiazolyl, triazolyl, oxadiazolyl, thiadiazolyl and tetrazolyl. In certain embodiments, R^{A} is a 6-membered heteroaryl group. Exemplary 6-membered heteroaryl groups include, but are not limited to, pyridinyl, pyridazinyl, pyrimidinyl, pyrazinyl, triazinyl and tetrazinyl. In certain embodiments, R^{A} is a 5,6-bicyclic heteroaryl group. Exemplary 5,6-bicyclic heteroaryl groups include, without limitation, indolyl, isoindolyl, indazolyl, benztriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. In certain embodiments, R^{A} is a 6,6-bicyclic heteroaryl group. Exemplary 6,6-bicyclic heteroaryl groups include, but are not limited to, naphthyridinyl, pteridinyl, quinolinyl, isoquinolinyl, cinnolinyl, quinoxalinyl, phthalazinyl and quinazolinyl.

In certain embodiments, R^{A} is a group of the formula (i)

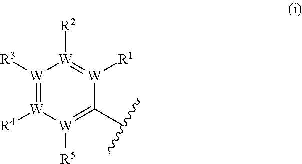

wherein each group W—R¹, W—R², W—R³, W—R⁴, and W—R⁵ independently represents either a nitrogen atom (N) or C—R¹, C—R², C—R³, C—R⁴, or C—R⁵, respectively; and wherein R¹, R², R³, R⁴ and R⁵ are independently selected from hydrogen, halogen, —CN, —NO₂, —N₃, —SO₂H, —SO₃H, —OH, —OR^{A1}, —ONR^{A2}₂, —NR^{A2}₂, —N(OR^{A3})R^{A3}, —SH, —SR^{A1}, —SSR^{A3}, —C(═O)R^{A1}, —CO₂H, —CHO, —C(OR^{A3})₂, —CO₂R^{A1}, —OC(═O)R^{A1}, —OCO₂R^{A1}, —C(═O)NR^{A2}₂, —OC(═O)NR^{A2}₂, —NR^{A2}C(═O)R^{A1}, —NR^{A2}CO₂R^{A1}, —NR^{A2}C(═O)NR^{A2}₂, —C(═NR^{A2})OR^{A1}, —OC(═NR^{A2})R^{A1}, —OC(═NR^{A2})OR^{A1}, —C(═NR^{A2})NR^{A2}₂, —OC(═NR^{A2})NR^{A2}₂,—NR^{A2}C(═NR^{A2})NR^{A2}₂, —C(═O)NR^{A2}SO₂R^{A1}, —NR^{A2}SO₂R^{A1}, —SO₂NR^{A2}₂, —SO₂R^{A1}, —SO₂OR^{A1}, —OSO₂R^{A1}, —S(═O)R^{A1}, —OS(═O)R^{A1}, —Si(R^{A1})₃, —OSi(R^{A1})₃—C(═S)NR^{A2}₂, —C(═O)SR^{A1}, —C(═S)SR^{A1}, —SC(═S)SR^{A1}, —P(═O)₂R^{A1}, —OP(═O)₂R^{A1}, —P(═O)(R^{A1})₂, —OP(═O)(R^{A1})₂, —OP(═O)(OR^{A3})₂, —P(═O)₂NR^{A2}₂, —OP(═O)₂NR^{A2}₂, —P(═O)(NR^{A2})₂, —OP(═O)(NR^{A2})₂, —NR^{A2}P(═O)(OR^{A3})₂, —NR^{A2}P(═O)(NR^{A2})₂, —P(R^{A3})₂, P(R^{A3})₃, —OP(R^{A3})₂, —OP(R^{A3})₃, —B(OR^{A3})₂, —BR^{A1}(OR^{A3}), C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; or one or more of R¹ and R², R² and R³, R³ and R⁴ Or R⁴ and R⁵ are joined to form a C₃₋₁₀ carbocyclyl, 3-14 membered heterocyclyl, C₆₋₁₄ aryl or 5-14 membered heteroaryl ring; each R^{A1} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each R^{A2} is, independently, selected from hydrogen, —OH, —OR^{A1}, —NR^{A3}₂, —CN, —C(═O)R^{A1}, —C(═O)NR^{A3}₂, —CO₂R^{A1}, —SO₂R^{A1}, —C(═NR^{A3})OR^{A1}, —C(═NR^{A3})NR^{A3}₂, —SO₂NR^{A3}₂, —SO₂R^{A3}, —SO₂OR^{A3}, —SOR^{A1}, —C(═S)NR^{A3}₂, —C(═O)SR^{A3}, —C(═S)SR^{A3}, —P(═O)₂R^{A1}, —P(═O)(R^{A1})₂, —P(═O)₂NR^{A3}₂, —P(═O)(NR^{A3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{A2} groups are joined to form a 3-14 membered heterocyclyl or 5-14 membered heteroaryl ring; and each R^{A3} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{A3} groups are joined to form a 3-14 membered heterocyclyl or 5-14 membered heteroaryl ring. In certain embodiments, the group of formula (i) represents a C₆₋₁₄ aryl group or a 6-14 membered heteroaryl group. In certain embodiments, the group of formula (i) represents a 6-14 membered heteroaryl group. In certain embodiments, the group of formula (i) represents a C₆₋₁₄ aryl group. In certain embodiments, the C₆₋₁₄ aryl group of formula (i) represents a phenyl group.

As used herein, when one or more of R¹, R², R³, R⁴ and R⁵ is referred to as “not hydrogen”, it is meant that one or more of R¹, R², R³, R⁴ and R⁵ is independently selected from halogen, —CN, —NO₂, —N₃, —SO₂H, —SO₃H, —OH, —OR^{A1}, —ONR^{A2}₂, —NR^{A2}₂, —N(OR^{A3})R^{A3}, —SH, —SR^{A1}, —SSR^{A3}, —C(═O)R^{A1}, —CO₂H, —CHO, —C(OR^{A3})₂, —CO₂R^{A1}, —OC(═O)R^{A1}, —OCO₂R^{A1}, —C(═O)NR^{A2}₂, —OC(═O)NR^{A2}₂, —NR^{A2}C(═O)R^{A1}, —NR^{A2}CO₂R^{A1}, —NR^{A2}C(═O) NR^{A2}₂, —C(═NR^{A2})OR^{A1}, —OC(═NR^{A2}) R^{A1}, —OC(═NR^{A2})OR^{A1}, —C(═NR^{A2})NR^{A2}₂, —OC(═NR^{A2})NR^{A2}₂, —NR^{A2}C(═NR^{A2})NR^{A2}₂, —C(═O)NR^{A2}SO₂R^{A1}, —NR^{A2}SO₂R^{A1}, —SO₂NR^{A2}₂, —SO₂R^{A1}, —SO₂OR^{A1}, —OSO₂R^{A1}, —S(═O)R^{A1}, —OS(═O) R^{A1}, —Si(R^{A1})₃, —OSi(R^{A1})₃—C(═S)NR^{A2}₂, —C(═O)SR^{A1}, —C(═S)SR^{A1}, —SC(S)SR^{A1}, —P(═O)₂R^{A1}, —OP(═O)₂R^{A1}, —P(═O)(R^{A1})₂, —OP(═O)(R^{A1})₂, —OP(═O)(OR^{A3})₂, —P(═O)₂NR^{A2}₂, —OP(═O)₂NR^{A2}₂, —P(═O)(NR^{A2})₂, —OP(═O)(NR^{A2})₂, —NR^{A2}P(═O)(OR^{A3})₂, NR^{A2}P(═O)(NR^{A2})₂, —P(R^{A3})₂, —P(R^{A3})₃, —OP(R^{A3})₂, —OP(R^{A3})₃, —B(OR^{A3})₂, or —BR^{A1}(OR^{A3}), C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; or one or more of R¹ and R², R² and R³, R³ and R⁴ Or R⁴ and R⁵ are joined to form a C₃₋₁₀ carbocyclyl, 3-14 membered heterocyclyl, C₆₋₁₄ aryl or 5-14 membered heteroaryl ring. In certain embodiments, R¹, R², R³, R⁴ and R⁵ are independently selected from hydrogen, halogen, —CN, —NO₂, —SO₂H, —SO₃H, —OH, —OR^{A1}, —NR^{A2}₂, —C(═O)R^{A1}, —CO₂H, —CHO, —C(OR^{A3})₂, —CO₂R^{A1}, —OC(═O)R^{A1}, —OCO₂R^{A1}, —C(═O)NR^{A2}₂, —OC(═O)NR^{A2}₂, —NR^{A2}C(═O)R^{A1}, NR^{A2}CO₂R^{A1}, —NR^{A2}C(═NR^{A2})₂, —C(═NR^{A2})OR^{A1}, —OC(═NR^{A2})R^{A1}, —OC(═NR^{A2})OR^{A1}, —C(═NR^{A2})NR^{A2}₂, —OC(═NR^{A2})NR^{A2}₂, —NR^{A2}C(═NR^{A2})NR^{A2}₂, —C(═O)NR^{A2}SO₂R^{A1}, —NR^{A2}SO₂R^{A1}, —SO₂NR^{A2}₂, —SO₂R^{A1}, —SO₂OR^{A1}, —OSO₂R^{A1}, —S(═O)R^{A1}, —OS(═O)R^{A1}, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; or one or more of R¹ and R², R² and R³, R³ and R⁴ Or R⁴ and R⁵ are joined to form a C₃₋₁₀carbocyclyl, 3-14 membered heterocyclyl, C₆₋₁₄ aryl or 5-14 membered heteroaryl ring. In certain embodiments, R¹, R², R³, R⁴ and R⁵ are independently selected from hydrogen, halogen, —CN, —OR^{A1}, —NR^{A2}₂, —CO₂H, —CO₂R^{A1}, —C(═O)NR^{A2}₂, —SO₂R^{A1}, C₁₋₁₀ alkyl, C₂₋₁₀ alkynyl, 3-14 membered heterocyclyl, and C₆₋₁₄ aryl; or one or more of R¹ and R², R² and R³, R³ and R⁴ or R⁴ and R⁵ are joined to form a 5-14 membered heteroaryl ring. In certain embodiments, R¹, R², R³, R⁴ and R⁵ are independently selected from hydrogen, halogen, —OR^{A1}, —NR^{A2}₂, —CO₂H, —C(═O)NR^{A2}₂, —SO₂R^{A1}, C₁₋₁₀ alkyl, 3-14 membered heterocyclyl; or R⁴ and R⁵ are joined to form a 5-14 membered heteroaryl ring. In certain embodiments, R¹, R², R³, R⁴ and R⁵ are independently selected from hydrogen, halogen, —OR^{A1}, C₁₋₁₀ alkyl, and —C(═O)NR^{A2}₂; or R⁴ and R⁵ are joined to form a 5-14 membered heteroaryl ring. In certain embodiments, R¹, R², R³, R⁴ and R⁵ are independently selected from hydrogen, halogen, —OR^{A1}, and —C(═O)NR^{A2}₂; or R⁴ and R⁵ are joined to form a 5-14 membered heteroaryl ring. In certain embodiments, R¹, R², R³, R⁴ and R⁵ are independently selected from hydrogen, halogen, and —OR^{A1}. In certain embodiments, R¹, R², R³, R⁴ and R⁵ are independently selected from hydrogen, fluoro, chloro, and —OR^{A1}. In certain embodiments, R¹, R², R³, R⁴ and R⁵ are independently selected from hydrogen, fluoro, chloro, and —OMe. In certain embodiments, R¹, R², R³, R⁴ and R⁵are independently selected from hydrogen, fluoro and —OR^{A1}. In certain embodiments, R¹, R², R³, R⁴ and R⁵ are independently selected from hydrogen, fluoro and —OMe. In certain embodiments, R¹, R², R³, R⁴ and R⁵ are independently selected from hydrogen and fluoro. In certain embodiments, R¹, R², R³, R⁴ and R⁵ are independently selected from hydrogen and chloro. In certain embodiments, R⁴ and R⁵ are joined to form a 5-14 membered heteroaryl ring.

In other embodiments, R^{A} is a group of the formula (ii):

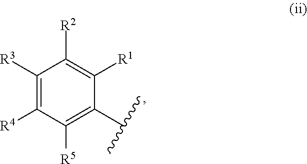

wherein R¹, R², R³, R⁴ and R⁵ are as defined above and herein. In certain embodiments, the group of formula (ii) represents a C₆₋₁₄ aryl group or a 6-14 membered heteroaryl group. In certain embodiments, the group of formula (ii) represents a 6-14 membered heteroaryl group. In certain embodiments, the group of formula (ii) represents a C₆₋₁₄ aryl group. In certain embodiments, the C₆₋₁₄ aryl group of formula (ii) represents a phenyl group. In certain embodiments, R^{A} is a monosubstituted, disubstituted or trisubstituted group of the formula (ii). In certain embodiments, R^{A} is a monosubstituted or disubstituted group of the formula (ii). In certain embodiments, R^{A} is a monosubstituted group of the formula (ii). For example, in certain embodiments, R^{A} is an ortho-substituted group of the formula (ii), e.g., wherein R¹-R⁴ are hydrogen, and R⁵ is not hydrogen. In certain embodiments, R^{A} is a meta-substituted group of the formula (ii), e.g., wherein R¹-R³ and R⁵ are hydrogen and R⁴ is not hydrogen. In certain embodiments, R^{A} is a para-substituted group of the formula (ii), e.g., wherein R¹, R², R⁴ and R⁵are hydrogen and R³ is not hydrogen. In certain embodiments, R^{A} is a disubstituted group of the formula (ii). For example, in certain embodiments, R^{A} is a 2,6-disubstituted group of the formula (ii), e.g., wherein R², R³ and R⁴ are hydrogen, and R¹ and R⁵ are not hydrogen. In certain embodiments, R^{A} is a 2,5-disubstituted group of the formula (ii), e.g., wherein R², R³ and R⁵ are hydrogen, and R¹ and R⁴ are not hydrogen. In certain embodiments, R^{A} is a 2,4-disubstituted group of the formula (ii), e.g., wherein R², R³ and R⁵are hydrogen, and R¹ and R³ are not hydrogen. In certain embodiments, R^{A} is a 2,3-disubstituted group of the formula (ii), e.g., wherein R¹, R² and R³ are hydrogen, and R⁴ and R⁵ are not hydrogen. In certain embodiments, R^{A} is a 3,4-disubstituted group of the formula (ii), e.g., wherein R¹, R⁴ and R⁵ are hydrogen, and R² and R³ are not hydrogen. In certain embodiments, R^{A} is a 3,5-disubstituted group of the formula (ii), e.g., wherein R¹, R³ and R⁵ are hydrogen, and R² and R⁴ are not hydrogen.

In certain embodiments, one of R¹ and R⁵ is halogen, —CN, —OR^{A1}, —NR^{A2}₂, —CO₂H, —CO₂R^{A1}, —C(═O)NR^{A2}₂, —SO₂R^{A1}, C₁₋₁₀ alkyl, C₂₋₁₀ alkynyl, 3-14 membered heterocyclyl, and C₆₋₁₄ aryl, and the other of R¹ and R⁵ is halogen, —CN, —OR^{A1}, —NR^{A2}₂, —C₂H, —CO₂R^{A1}, —C(═O)NR^{A2}₂, —SO₂R^{A1}, C₁₋₁₀ alkyl, C₂₋₁₀ alkynyl, 3-14 membered heterocyclyl, and C₆₋₁₄ aryl. In certain embodiments, one of R¹ and R⁵ is halogen, —OR^{A1}, C₁₋₁₀ alkyl, or —C(═O)NR^{A2}₂, and the other of R¹ and R⁵ is halogen, —OR^{A1}, C₁₋₁₀ alkyl, or —C(═O)NR^{A2}₂. In certain embodiments, each of R¹ and R⁵ is independently halogen. For example, each of R¹ and R⁵ is independently selected from fluoro and chloro. In certain embodiments, R^{A} is a trisubstituted group of the formula (ii).

For example, in certain embodiments, R^{A} is a 2,4,6-trisubstituted group of the formula (ii), e.g., wherein R² and R⁴ are hydrogen, and R¹, R³ and R⁵ are not hydrogen. In certain embodiments, R^{A} is a 2,3,6-trisubstituted group of the formula (ii), e.g., wherein R² and R³ are hydrogen, and R¹, R⁴ and R⁵ are not hydrogen. In certain embodiments, R^{A} is a 2,4,5-trisubstituted group of the formula (ii), e.g., wherein R² and R⁵ are hydrogen, and R¹, R³ and R⁴ are not hydrogen. In certain embodiments, R^{A} is a 2,3,4-trisubstituted group of the formula (ii), e.g., wherein R⁴ and R⁵ are hydrogen, and R¹, R² and R³ are not hydrogen. In certain embodiments, R^{A} is a 3,4,5-trisubstituted group of the formula (ii), e.g., wherein R¹ and R⁵ are hydrogen, and R², R³ and R⁴ are not hydrogen. In certain embodiments, R^{A} is heteroaryl selected from a 5-6-membered heteroaryl, a 5,6-bicyclic heteroaryl or a 6,6-bicyclic heteroaryl. In certain embodiments, R^{A} is a 6-membered heteroaryl. In certain embodiments, R^{A} is a 6-membered heteroaryl selected from pyridinyl.

In certain embodiments, R^{A} is 2-pyridinyl, 3-pyridinyl or 4-pyridinyl. In certain embodiments, R^{A} is a 2-pyridinyl wherein W—R¹ is N, and W—R², W—R³, W—R⁴, and W—R⁵ are C—R², C—R³, C—R⁴ and C—R⁵, respectively, e.g.,

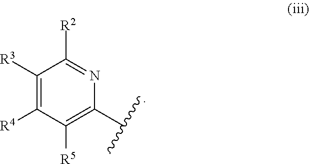

In certain embodiments, R^{A} is a 3-pyridinyl wherein W—R² is N, and W—R¹, W—R³, W—R⁴, and W—R⁵ are C—R¹, C—R³, C—R⁴ and C—R⁵, respectively, e.g.,

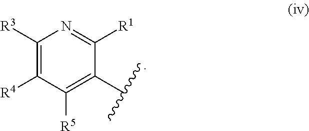

In certain embodiments, R^{A} is a 4-pyridinyl wherein W—R³ is N and W—R¹, W—R², W—R⁴, and W—R⁵ are C—R¹, C—R², C—R⁴ and C—R⁵, respectively, e.g.,

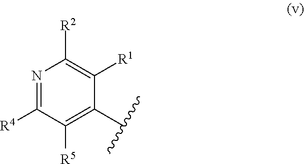

wherein R¹, R², R³, R⁴ and R⁵ are as defined above and herein.

In certain embodiments, R^{A} is a monosubstituted or disubstituted pyridinyl. In certain embodiments, R^{A} is a monosubstituted pyridinyl. In certain embodiments, R^{A} is a monosubstituted pyridinyl of the formula (iii) wherein R³, R⁴, R⁵ are hydrogen and R² is not hydrogen. In certain embodiments, R^{A} is a monosubstituted pyridinyl of the formula (iii) wherein R², R⁴, R⁵ are hydrogen and R³ is not hydrogen. In certain embodiments, R^{A} is a monosubstituted pyridinyl of the formula (iii) wherein R², R³, R⁵ are hydrogen and R⁴ is not hydrogen. In certain embodiments, R^{A} is a monosubstituted pyridinyl of the formula (iii) wherein R², R³, R⁴ are hydrogen and R⁵ is not hydrogen. In certain embodiments, R^{A} is a monosubstituted pyridinyl of the formula (iv) wherein R³, R⁴, R⁵ are hydrogen and R¹ is not hydrogen. In certain embodiments, R^{A} is a monosubstituted pyridinyl of the formula (iv) wherein R¹, R⁴, R⁵ are hydrogen and R³ is not hydrogen. In certain embodiments, R^{A} is a monosubstituted pyridinyl of the formula (iv) wherein R¹, R³, R⁵ are hydrogen and R⁴ is not hydrogen. In certain embodiments, R^{A} is a monosubstituted pyridinyl of the formula (iv) wherein R¹, R³, R⁴ are hydrogen and R⁵ is not hydrogen. In certain embodiments, R^{A} is a monosubstituted pyridinyl of the formula (v) wherein R², R⁴, R⁵ are hydrogen and R¹ is not hydrogen. In certain embodiments, R^{A} is a monosubstituted pyridinyl of the formula (v) wherein R¹, R⁴, R⁵ are hydrogen and R² is not hydrogen. In certain embodiments, R^{A} is a disubstituted pyridinyl. In certain embodiments, R^{A} is a disubstituted pyridinyl of the formula (iii) wherein R³ and R⁴ are hydrogen and R² and R⁵ are not hydrogen. In certain embodiments, R^{A} is a disubstituted pyridinyl of the formula (iii) wherein R² and R⁴ are hydrogen and R³ and R⁵ are not hydrogen. In certain embodiments, R^{A} is a disubstituted pyridinyl of the formula (iii) wherein R² and R³ are hydrogen and R⁴ and R⁵ are not hydrogen. In certain embodiments, R^{A} is a disubstituted pyridinyl of the formula (iii) wherein R³ and R⁵ are hydrogen and R² and R⁴ are not hydrogen. In certain embodiments, R^{A} is a disubstituted pyridinyl of the formula (iii) wherein R⁴ and R⁵ are hydrogen and R² and R³ are not hydrogen. In certain embodiments, R^{A} is a disubstituted pyridinyl of the formula (iii) wherein R² and R⁵ are hydrogen and R³ and R⁴ are not hydrogen. In certain embodiments, R^{A} is a disubstituted pyridinyl of the formula (iv) wherein R³ and R⁴ are hydrogen and R¹ and R⁵ are not hydrogen. In certain embodiments, R^{A} is a disubstituted pyridinyl of the formula (iv) wherein R³ and R⁵ are hydrogen and R¹ and R⁴ are not hydrogen. In certain embodiments, R^{A} is a disubstituted pyridinyl of the formula (iv) wherein R⁴ and R⁵ are hydrogen and R¹ and R³ are not hydrogen. In certain embodiments, R^{A} is a disubstituted pyridinyl of the formula (iv) wherein R¹ and R⁴are hydrogen and R³ and R⁵are not hydrogen. In certain embodiments, R^{A} is a disubstituted pyridinyl of the formula (iv) wherein R¹ and R⁵ are hydrogen and R³ and R⁴ are not hydrogen. In certain embodiments, R^{A} is a disubstituted pyridinyl of the formula (iv) wherein R¹ and R³ are hydrogen and R⁴ and R⁵ are not hydrogen. In certain embodiments, R^{A} is a disubstituted pyridinyl of the formula (v) wherein R² and R⁴ are hydrogen and R¹ and R⁵ are not hydrogen. In certain embodiments, R^{A} is a disubstituted pyridinyl of the formula (v) wherein R⁴ and R⁵ are hydrogen and R¹ and R² are not hydrogen. In certain embodiments, R^{A} is a disubstituted pyridinyl of the formula (v) wherein R² and R⁵ are hydrogen and R¹ and R⁴ are not hydrogen. In certain embodiments, R^{A} is a disubstituted pyridinyl of the formula (v) wherein R¹ and R⁵ are hydrogen and R² and R⁴ are not hydrogen.

In certain embodiments, R^{A} is a 5,6-bicyclic heteroaryl. For example, in certain embodiments, R^{A} is a 5,6-bicyclic heteroaryl group of the formula (vi):

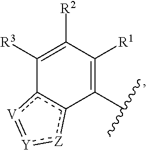

wherein R¹, R², R³are as defined above and herein and R⁴ and R⁵ are joined to form a 5-membered heteroaryl ring; V, Y and Z are independently selected from CR^{A4}, O, S, N, or NR^{A5}; each R^{A4} is, independently, selected from hydrogen, halogen, —CN, —NO₂, —N₃, —SO₂H, —SO₃H, —OH, —OR^{A6}, —ONR^{A7}₂, —NR^{A7}₂, —N(OR^{A6})R^{A8}, —SH, —SR^{A6}, —SSR^{A8}, —C(═O)R^{A6}, —CO₂H, —CHO, —C(OR^{A8})₂, —CO₂R^{A6}, —OC(═O)R^{A6}, —OCO₂R^{A6}, —C(═O)NR^{A7}₂, —OC(═O)NR^{A7}₂, —NR^{A7}C(═O)R^{A6}, —NR^{A7}CO₂R^{A6}, —NR^{A7}C(═O)NR^{A7}₂, —C(═NR^{A7})OR^{A6}, —OC(═NR^{A7})R^{A6}, —OC(═NR^{A7})OR^{A6}, —C(═NR^{A7})NR^{A7}₂, —OC(═NR^{A7})NR^{A7}₂, —NR^{A7}C(═NR^{A7})NR^{A7}₂, —C(═O)NR^{A7}SO₂R^{A6}, —NR^{A7}SO₂R^{A6}, —SO₂NR^{A7}₂, —SO₂R^{A6}, —SO₂OR^{A6}, —OSO₂R^{A6}, —S(═O)R^{A6}, —OS(═O)R^{A6}, —Si(R^{A6})₃, —OSi(R^{A6})₃—C(═S)NR^{A7}₂, —C(═O)SR^{A6}, —C(═S)SR^{A6}, —SC(═S)SR^{A6}, —P(═O)₂R^{A6}, —OP(═O)₂R^{A6}, —P(═O)(R^{A6})₂, —OP(═O)(R^{A6})₂, —OP(═O)(OR^{A8})₂, —P(═O)₂NR^{A7}₂, —OP(═O)₂NR^{A7}₂, —P(═O)(NR^{A7})₂, —OP(═O)(NR^{A7})₂, —NR^{A7}P(═O)(OR^{A8})₂, —NR^{A7}P(═O)(NR^{A7})₂, —P(R^{A8})₂, —P(R^{A8})₃, —OP(R^{A8})₂, —OP(R^{A8})₃, —B(OR^{A8})₂, or —BR^{A6}(OR^{A8}), C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each R^{A6} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each R^{A5} and R^{A7} is, independently, selected from hydrogen, —OH, —OR^{A6}, —NR^{A7}₂, —CN, —C(═O)R^{A6}, —C(═O)NR^{A7}₂, —CO₂R^{A6}, —SO₂R^{A7}, —C(═NR^{A3})OR^{A6}, —C(═NR^{A7})NR^{A7}₂, —SO₂NR^{A3}₂, —SO₂R^{A6}, —SO₂OR^{A8}, —SOR^{A6}, —C(═S)NR^{A7}₂, —C(═O)SR^{A8}, —C(═S)SR^{A8}, —P(═O)₂R^{A6}, —P(═O)(R^{A6})₂, —P(═O)₂NR^{A8}₂, P(═O)(NR^{A8})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{A7} groups are joined to form a 3-14 membered heterocyclyl or 5-14 membered heteroaryl ring; each R^{A8} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{A8} groups are joined to form a 3-14 membered heterocyclyl or 5-14 membered heteroaryl ring; and the dashed line represents a double or single bond.

In certain embodiments, R¹ is hydrogen. In certain embodiments, R² is hydrogen. In certain embodiments, R³ is hydrogen. In certain embodiments, R¹, R² and R³ are hydrogen.

In certain embodiments, R^{A} is a heteroaryl group of

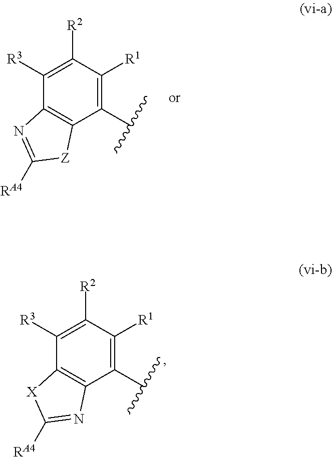

wherein R¹, R², R³ are as defined above and herein and V and Z are independently selected from O, S and NR^{A5}. In certain embodiments, wherein R^{A} is a heteroaryl group of the formulae (vi-a) or (vi-b), V and Z are O (i.e., benzoxazolyl). In certain embodiments, V and Z are S (i.e., benzthiazolyl). In certain embodiments, V and Z are NR^{A5} (i.e., imidazolyl). In certain embodiments, R^{A} is a heteroaryl group of

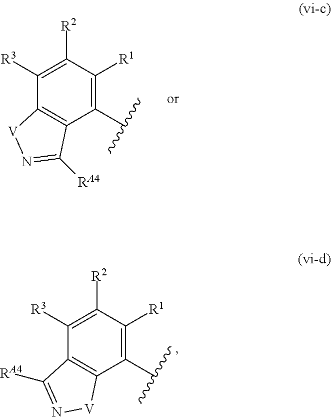

wherein R¹, R², R³are as defined above and herein and V is independently selected from O, S and NR^{A5}. In certain embodiments, wherein R^{A} is a heteroaryl group of the formulae (vi-c) or (vi-d), V is O (i.e., benzisoxazolyl). In certain embodiments, V is S (i.e., benzisothiazolyl). In certain embodiments, V is NR^{A5} (i.e., indazolyl). In certain embodiments, R^{A} is a heteroaryl group of the

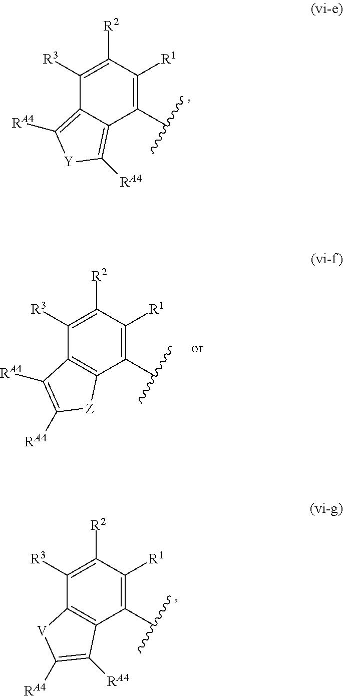

wherein R¹, R², R³ and R^{A4} are as defined above and herein and V, Y and Z are independently selected from O, S and NR^{A5}. In certain embodiments, wherein R^{A} is a heteroaryl group of the formulae (vi-e), (vi-f) or (vi-g), Y is O (i.e., benzofuranyl or isobenzofuranyl). In certain embodiments, Y is S (i.e., benzothiophenyl or isobenzothiophenyl). In certain embodiments, Y is NR^{A5} (i.e., indolyl or isoindolyl). In certain embodiments, R^{A} is a heteroaryl group of

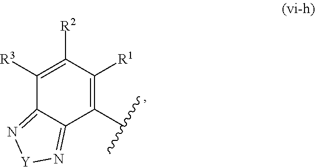

wherein R¹, R², R³ are as defined above and herein and Y is independently selected from O, S and NR^{A5}. In certain embodiments, wherein R^{A} is a heteroaryl group of the formula (vi-e), Y is O (i.e., benzoxadiazolyl). In certain embodiments, Y is S (i.e., benzthiadiazolyl). In certain embodiments, Y is NR^{A5} (i.e., benztriazolyl).

As described generally above, X is selected from hydrogen, —CN, —CHO, —C(═O)R^{X1}, C(═O)NR^{X2}₂, —CO₂H, CO₂R^{X1}, —SO₂R^{X1}, —C(═NR^{X2})OR^{X1}, —C(═NR^{X2})NR^{X2}₂, —SO₂NR^{X2}₂, —SO₂R^{X1}, —SO₃H, —SO₂OR^{X1}, —SOR^{X1}, —C(═S)NR^{X2}₂, —C(═O)SR^{X1}, —C(═S)SR^{X1}, —P(═O)₂R^{X1}, —P(═O)(R^{X1})₂), —P(═O)₂NR^{X2}₂, —P(═O)(NR^{X2})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl; or X and R^{A}, together with the carbon atoms to which each is attached, are joined to form a 5-10 membered carbocyclyl, heterocyclyl, aryl or heteroaryl ring; each R^{X1} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each R^{X2} is, independently, selected from hydrogen, —OH, —OR^{X1}, —NR^{X3}₂, —CN, —C(═O)R^{X1}, —C(═O)NR^{X3}₂, —CO₂R^{X1}, —SO₂R^{X1}, —C(═NR^{X3})OR^{X1}, —C(═NR^{X3})NR^{X3}₂, —SO₂NR^{X3}₂, —SO₂R^{X3}, —SO₂OR^{X3}, —SOR^{X1}, —C(═S)NR^{X3}₂, —C(═O)SR^{X3}, —C(═S)SR^{X3}, —P(═O)₂R^{X1}, —P(═O)(R^{X3})₂), —P(═O)₂NR^{X3}₂, —P(═O)(NR^{X3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; and each R^{X3} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl. In certain embodiments, X is selected from hydrogen, —CN, —CHO, —C(═O)R^{X1}, —C(═O)NR^{X2}₂, —CO₂H, CO₂R^{X}′, —SO₂R^{X1}, —C(═NR^{X2})OR^{X1}, —C(═NR^{X2})NR^{X2}₂, —SO₂NR^{X2}₂, —SO₂R^{X1}, —SO₃H, —SO₂OR^{X1}, —SOR^{X1}, —C(═S)NR^{X2}₂, —C(═O)SR^{X1}, —C(═S)SR^{X1}, —P(═O)₂R^{X1}, —P(═O)(R^{X1})₂, —P(═O)₂NR^{X2}₂, —P(═O)(NR^{X2})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl. In certain embodiments, X is selected from hydrogen, —CN, —CHO, —C(═O)R^{C1}, —C(═O)NR^{C2}₂, —CO₂H, —CO₂R^{C1}, —C(═NR^{C2})OR^{C1}, —C(═NR^{C2})NR^{C2}₂, —C(═S)NR^{C2}₂, —C(═O)SR^{C1}, —C(═S)SR^{C1}, C₁₋₁₀ perhaloalkyl, C₆₋₁₄ aryl, and 5-14 membered heteroaryl. In certain embodiments, X is selected from hydrogen, —CN, —C(═O)NR^{X2}₂, —CO₂R^{X1}, and, C₆₋₁₄ aryl. In certain embodiments, X is selected from —CN, —C(═O)NR^{X2}₂, and —CO₂R^{X1}. In certain embodiments, X is —CN.

As described generally above, in certain embodiments, R^{A} and X, together with the carbon atoms to which each is attached, are joined to form a 5-10 membered ring. For example, in certain embodiments, R^{A} and X, together with the carbon atoms to which each is attached, are joined to form a ring of the formula (i-b):

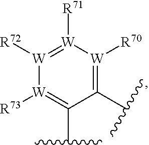

wherein each group W—R⁷⁰, W—R⁷¹, W—R⁷², and W—R⁷³ independently represents either a nitrogen atom (N) or C—R⁷⁰, C—R⁷¹, C—R⁷², or C—R⁷³, respectively; and wherein R⁷⁰, R⁷¹, R⁷² and R⁷³ are independently selected from hydrogen, halogen, —CN, —NO₂, —N₃, —SO₂H, —SO₃H, —OH, —OR^{A9}, —ONR^{A10}₂, —NR^{A10}₂, —N(OR^{A11})R^{A11}, —SH, —SR^{A9}, —SSR^{A11}, —C(═O)R^{A9}, —CO₂H, —CHO, —C(OR^{A11})₂, —CO₂R^{A9}, —OC(═O)R^{A9}, —OCO₂R^{A9}, —C(═O)NR^{A10}₂, —OC(═O)NR^{A10}₂, —NR^{A10}C(═O)R^{A9}, —NR^{A10}CO₂R^{A9}, —NR^{A10}C(═O)NR^{A10}₂, —C(═NR^{A10})OR^{A9}, —OC(═NR^{A10})R^{A9}, —OC(═NR^{A10})OR^{A9}, —C(═NR^{A10})NR^{A10}₂, —OC(═NR^{A10})NR^{A10}₂, —NR^{A10}C(═NR^{A10})NR^{A10}₂, —C(═O)NR^{A10}SO₂R^{A9}, —NR^{A10}SO₂R^{A9}, —SO₂NR^{A10}₂, —SO₂R^{A9}, —SO₂OR^{A9}, —OSO₂R^{A9}, —S(═O)R^{A9}, —OS(═O)R^{A9}, —Si(R^{A9})₃, —OSi(R^{A9})₃—C(═S)NR^{A10}₂, —C(═O)SR^{A9}, —C(═S)SR^{A9}, —SC(═S)SR^{A9}, —P(═O)₂R^{A9}, —OP(═O)₂R^{A9}, —P(═O)(R^{A9})₂, —OP(═O)(R^{A9})₂, —OP(═O)(OR^{A11})₂, —P(═O)₂NR^{A10}₂, —OP(═O)₂NR^{A10}₂, —P(═O)(NR^{A10})₂, —OP(═O)(NR^{A0})₂, —NR^{A10}P(═O)(OR^{A11})₂, —NR^{A10}P(═O)(NR^{A10})₂, —P(R^{A11})₂, —P(R^{A11})₃, —OP(R^{A11})₂, —OP(R^{A11})₃, —B(OR^{A11})₂, or —BR^{A9}(OR^{A11}), C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; or one or more of R¹ and R², R² and R³, R³ and R⁴ Or R⁴ and R⁵ are joined to form a C₃₋₁₀ carbocyclyl, 3-14 membered heterocyclyl, C₆₋₁₄ aryl or 5-14 membered heteroaryl ring; each R^{A9} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each R^{A10} is, independently, selected from hydrogen, —OH, —OR^{A9}, —NR^{A11}₂, —CN, —C(═O)R^{A9}, —C(═O)NR^{A11}₂, —CO₂R^{A9}, —SO₂R^{A9}, —C(═NR^{A11})OR^{A9}, —C(═NR^{A11})NR^{A11}₂, —SO₂NR^{A11}₂, —SO₂R^{A11}, —SO₂OR^{A11}, —SOR^{A9}, —C(═S)NR^{A11}₂, —C(═O)SR^{A11}, —C(═S)SR^{A11}, —P(═O)₂R^{A9}, —P(═O)(R^{A9})₂, —P(═O)₂NR^{A11}2, —P(═O)(NR^{A11})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{A10} groups are joined to form a 3-14 membered heterocyclyl or 5-14 membered heteroaryl ring; and each R^{A11} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{A11} groups are joined to form a 3-14 membered heterocyclyl or 5-14 membered heteroaryl ring.

In certain embodiments, each group W—R⁷⁰, W—R⁷¹, W—R⁷², and W—R⁷³ independently represents C—R⁷⁰, C—R⁷¹, C—R⁷², or C—R⁷³, respectively. In certain embodiments, one of the groups W—R⁷⁰, W—R⁷¹, W—R⁷², and W—R⁷³ represents a nitrogen atom (N). For example, each group W—R⁷⁰, W—R⁷¹, and W—R⁷² represents C—R⁷⁰, C—R⁷¹, C—R⁷², respectively, and W—R⁷³ represents a nitrogen atom (N).

As described generally above, R^{B} is selected from C₁₋₁₀ alkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl and 5-14 membered heteroaryl; or R^{B} and R^{C} together with the nitrogen (N) atom to which each is attached are joined to form a 5-14 membered carbocyclyl, heterocyclyl, aryl or heteroaryl ring. In certain embodiments, R^{B} is selected from C₁₋₁₀ alkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl and 5-14 membered heteroaryl. In certain embodiments, R^{B} is an acyclic group, i.e., selected from C₁₋₁₀ alkyl, C₂₋₁₀ alkenyl, C₂₋₁₀ alkynyl and 3-14 membered heteroaliphatic. In certain embodiments, R^{B} is C₁₋₁₀ alkyl. In certain embodiments, R^{B} is a substituted C₁₋₁₀ alkyl, e.g., a C₁₋₁₀ aralkyl group. In certain embodiments, R^{B} is a C₁₋₂ aralkyl, e.g., for example, a substituted or unsubstituted benzyl group (C₁ aralkyl) or substituted or unsubstituted phenylethyl group (C₂ aralkyl). In certain embodiments, R^{B} is a C₁₋₁₀ heteroaralkyl. In certain embodiments, R^{B} is alkenyl. In certain embodiments, R^{B} is alkynyl. In certain embodiments, R^{B} is 3-14 membered heteroaliphatic. Alternatively, in certain embodiments, R^{B} is a cyclic group, i.e., selected from C₃₋₁₀ carbocyclyl, 3-14 membered heterocyclyl, C₆₋₁₄ aryl and 5-14 membered heteroaryl. In certain embodiments, R^{B} is C₃₋₁₀ carbocyclyl or 3-14 membered heterocyclyl. In certain embodiments, R^{B} is C₃₋₁₀ carbocyclyl. Exemplary carbocyclyl groups include, but are not limited to, cyclopropyl (C₃), cyclobutyl (C₄), cyclopentyl (C₅), cyclopentenyl (C₅), cyclohexyl (C₆), cyclohexenyl (C₆), cyclohexadienyl (C₆), cycloheptyl (C₇), cycloheptadienyl (C₇), cycloheptatrienyl (C₇) and cyclooctyl (C₈). In certain embodiments, R^{B} is 3-14 membered heterocyclyl. Exemplary heterocyclyl groups include, but are not limited to, azirdinyl, oxiranyl, thiorenyl, azetidinyl, oxetanyl, thietanyl, tetrahydrofuranyl, dihydrofuranyl, tetrahydrothiophenyl, dihydrothiophenyl, pyrrolidinyl, dihydropyrrolyl, dioxolanyl, oxathiolanyl and dithiolanyl, piperidinyl, tetrahydropyranyl, dihydropyridinyl, thianyl, piperazinyl, morpholinyl, dithianyl, dioxanyl, azepanyl, oxepanyl thiepanyl, azocanyl, oxecanyl and thiocanyl. In certain embodiments, R^{B} is C₆₋₁₄ aryl or 5-14 membered heteroaryl. In certain embodiments, R^{B} is C₆₋₁₄ aryl. Exemplary aryl groups include, but are not limited to, phenyl, naphthyl and anthracyl. In certain embodiments, R^{B} is phenyl (C₆ aryl). In certain embodiments, R^{B} is unsubstituted phenyl. In certain embodiments, R^{B} is naphthyl (C₁₀ aryl). In certain embodiments, R^{B} is 5-14 membered heteroaryl. In certain embodiments, R^{B} is 5-10 membered heteroaryl. In certain embodiments, R^{B} is 5-6 membered heteroaryl. In certain embodiments, R^{B} is a 5,6-bicyclic heteroaryl. In certain embodiments, R^{B} is a 6,6-bicyclic heteroaryl. In certain embodiments, R^{B} is a 5-membered heteroaryl group. Exemplary 5-membered heteroaryl groups include, but are not limited to, pyrrolyl, furanyl, thiophenyl, imidazolyl, pyrazolyl, oxazolyl, isoxazolyl, thiazolyl, isothiazolyl, triazolyl, oxadiazolyl, thiadiazolyl and tetrazolyl. In certain embodiments, R^{B} is a 6-membered heteroaryl group. Exemplary 6-membered heteroaryl groups include, but are not limited to, pyridinyl, pyridazinyl, pyrimidinyl, pyrazinyl, triazinyl and tetrazinyl. In certain embodiments, R^{B} is a 5,6-bicyclic heteroaryl group. Exemplary 5,6-bicyclic heteroaryl groups include, but are not limited to, indolyl, isoindolyl, indazolyl, benztriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. In certain embodiments, R^{B} is a 6,6-bicyclic heteroaryl group. Exemplary 6,6-bicyclic heteroaryl groups include, but are not limited to, naphthyridinyl, pteridinyl, quinolinyl, isoquinolinyl, cinnolinyl, quinoxalinyl, phthalazinyl and quinazolinyl. In certain embodiments, R^{B} is substituted with the group -L-R^{D} wherein L is a covalent bond or a divalent C₁₋₁₀ hydrocarbon chain, wherein one, two or three methylene units of L are optionally and independently replaced with one or more —O—, —S—, —NR^{B8}—, —(C═NR^{B8})—, —C(═O)—, —C(═S)—, —S(═O)—, —S(═O)₂—, divalent C₃₋₁₀ carbocyclyl, divalent 3-14 membered heterocyclyl, divalent C₆₋₁₄ aryl or divalent 5-14 membered heteroaryl group; R^{D} is selected from —CN, —NO₂, —N₃, —SO₂H, —SO₃H, —C(═O)R^{B7}, —CO₂H, —CHO, —C(OR^{B9})₂, —CO₂R^{B7}, —OC(═O)R^{B7}, —OCO₂R^{B7}, —C(═O)NR^{B8}₂, —OC(═O)NR^{B8}₂, —NR^{B8}C(═O)R^{B7}, —NR^{B8}CO₂R^{B7}, —NR^{B8}C(═O)NR^{B8}₂, —C(═NR^{B8})OR^{B7}, —OC(═NR^{B8})R^{B7}, —OC(═NR^{B8})OR^{B7}, —C(═NR^{B8})NR^{B8}₂, —OC(═NR^{B8})NR^{B8}₂, —NR^{B8}C(═NR^{B8})NR^{B8}₂, —C(═O)NR^{B8}SO₂R^{B7}, —NR^{B8}SO₂R^{B7}, —SO₂NR^{B8}₂, —SO₂R^{B7}, —SO₂OR^{B7}, —OSO₂R^{B7}, —S(═O)R^{B7}, —OS(═O)R^{B7}, —C(═S)NR^{B8}₂, —C(═O)SR^{B7}, —C(═S)SR^{B7}, —SC(═S)SR^{B7}, —P(═O)₂R^{B7}, —OP(═O)₂R^{B7}, —P(═O)(R^{B7})₂, —OP(═O)(R^{B7})₂, —OP(═O)(OR^{B9})₂, —P(═O)₂NR^{B8}₂, —OP(═O)₂NR^{B8}₂, —P(═O)(NR^{B8})₂, —OP(═O)(NR^{B8})₂, —NR^{B8}P(═O)(OR^{B9})₂, —NR^{B8}P(═O)(NR^{B8})₂, —B(OR^{B9})₂, —BR^{B7}(OR^{B9}), and tetrazolyl; each R^{B7} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each R^{B8} is, independently, selected from hydrogen, —OH, —OR^{B7}, —NR^{B9}₂, —CN, —C(═O)R^{B7}, —C(═O)NR^{B9}₂, —CO₂R^{B7}, —SO₂R^{B7}, —C(═NR^{B9})OR^{B7}, —C(═NR^{B9})NR^{B9}₂, —SO₂NR^{B9}₂, —SO₂R^{B9}, —SO₂OR^{B9}, —SOR^{B7}, —C(═S)NR^{B9}₂, —C(═O)SR^{B9}, —C(═S)SR^{B9}, —P(═O)₂R^{B7}, —P(═O)(R^{B7})₂, —P(═O)₂NR^{B9}₂, —P(═O)(NR^{B9})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B8} groups are joined to form a 3-14 membered heterocyclyl or 5-14 membered heteroaryl ring; and each R^{B9} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B9} groups are joined to form a 3-14 membered heterocyclyl or 5-14 membered heteroaryl ring.

In certain embodiments, L is a covalent bond. In certain embodiments, L is a divalent C₁₋₁₀ hydrocarbon chain, wherein one, two or three methylene units of L are optionally and independently replaced with one or more —O—, —S—, —NR^{B8}—, —(C═NR^{B8})—, —C(═O)—, —C(═S)—, —S(═O)—, —S(═O)₂—, divalent carbocyclyl, divalent heterocyclyl, divalent aryl or divalent heteroaryl group. In certain embodiments, L is a divalent C₁₋₁₀ hydrocarbon chain, wherein one, two or three methylene units of L are optionally and independently replaced with one or more —O—, —S—, —NR^{B8}—, —(C═NR^{B8})—, —C(═O)—, —C(═S)—, —S(═O)—, —S(═O)₂—, divalent C₃₋₁₀ carbocyclyl, divalent 3-14 membered heterocyclyl, divalent C₆₋₁₄ aryl or divalent 5-14 membered heteroaryl group.

As generally described above, R^{D} is selected from —CN, —NO₂, —SO₂H, —SO₃H, —C(═O)R^{B7}, —CO₂H, —CHO, —C(OR^{B9})₂, —CO₂R^{B7}, —OC(═O)R^{B7}, —OCO₂R^{B7}, —C(═O)NR^{B8}₂, —OC(═O)NR^{B8}₂, —NR^{B8}C(═O)R^{B7}, —NR^{B8}CO₂R^{B7}, —NR^{B8}C(═O)NR^{B8}₂, —C(═NR^{B8})OR^{B7}, —OC(═NR^{B8})R^{B7}, —OC(═NR^{B8})OR^{B7}, —C(═NR^{B8})NR^{B8}₂, —OC(═NR^{B8})NR^{B8}₂, —NR^{B8}C(═NR^{B8})NR^{B8}₂), —C(═O)NR^{B8}SO₂R^{B7}, —NR^{B8}SO₂R^{B7}, —SO₂NR^{B8}₂, —SO₂R^{B7}, —SO₂OR^{B7}, —OSO₂R^{B7}, —S(═O)R^{B7}, —OS(═O)R^{B7}, —C(═S)NR^{B8}₂, —C(═O)SR^{B7}, —C(═S)SR^{B7}, —SC(═S)SR^{B7}, P(═O)₂R^{B7}, —OP(═O)₂R^{B7}, —P(═O)(R^{B7})₂, —OP(═O)(R^{B7})₂, —OP(═O)(OR^{B9})₂, —P(═O)₂NR^{B8}₂, OP(═O)₂NR^{B8}₂, —P(═O)(NR^{B8})₂, —OP(═O)(NR^{B8})₂, —NR^{B8}P(═O)(OR^{B9})₂, —NR^{B8}P(═O)(NR^{B8})₂, —B(OR^{B9})₂, —BR^{B7}(OR^{B9}) and tetrazolyl. However, in certain embodiments, R^{D} is not —CO₂R^{B7} (e.g., CO₂Me, CO₂Et, CO₂nPr, CO₂iPr, CO₂tBu), but can be selected from any of the other substituents listed above. In certain embodiments, R^{D} is not —C(═O)R^{B7}, but can be selected from any of the other substituents listed above. In certain embodiments, R^{D} is not —CHO, but can be selected from any of the other substituents listed above. In certain embodiments, R^{D} is not —C(OR^{B9})₂, but can be selected from any of the other substituents listed above. In certain embodiments, R^{D} is not —CN, but can be selected from any of the other substituents listed above. In certain embodiments, R^{D} is not —NO₂, but can be selected from any of the other substituents listed above. In certain embodiments, R^{D} is not any one of —SO₂H, —SO₃H, —SO₂NR^{B8}₂, —NR^{B8}SO₂R^{B7}, —SO₂R^{B7}, —SO₂OR^{B7}, —OSO₂R^{B7}, —S(═O)R^{B7} or —OS(═O)R^{B7}, but can be selected from any of the other substituents listed above. In certain embodiments, R^{D} is not any one of —OC(═O)R^{B7}, —OCO₂R^{B7}, —OC(═O)NR^{B8}₂, —NR^{B8}C(═O)R^{B7}, —NR^{B8}CO₂R^{B7}, —NR^{B8}C(═O)NR^{B8}₂, —OC(═NR^{B8}) R^{B7}, —OC(═NR^{B8})OR^{B7}, —OC(═NR^{B8})NR^{B8}₂ or —NR^{B8}C(═NR^{B8})NR^{B8}₂, but can be selected from any of the other substituents listed above. In certain embodiments, R^{D} is not any one of —C(═S)NR^{B8}₂, —C(═O)SR^{B7}, —C(═S)SR^{B7} or —SC(═S)SR^{B7}, but can be selected from any of the other substituents listed above. In certain embodiments, R^{D} is not any one of —P(═O)₂R^{B7}, —OP(═O)₂R^{B7}, —P(═O)(R^{B7})₂, —OP(═O)(R^{B7})₂, OP(═O)(OR^{B9})₂, —P(═O)₂NR^{B8}₂, —OP(═O)₂NR^{B8}₂, —P(═O)(NR^{B8})₂, —OP(═O)(NR^{B8})₂, —NR^{B8}P(═O)(OR^{B9})₂ or —NR^{B8}P(═O)(NR^{B8})₂, but can be selected from any of the other substituents listed above. In certain embodiments, R^{D} is not any one of —B(OR^{B9})₂ or —BR^{B7}(OR^{B9}), but can be selected from any of the other substituents listed above. In certain embodiments, R^{D} is not tetrazolyl, but can be selected from any of the other substituents listed above. In certain embodiments, R^{D} is selected from —CN, —NO₂, —SO₂H, —SO₃H, —C(═O)R^{B7}, —CO₂H, —CHO, —CO₂R^{B7}, —C(═O)NR^{B8}₂, —C(═NR^{B8})OR^{B7}, —C(═NR^{B8})NR^{B8}₂, C(═O)NR^{B8}SO₂R^{B7}, —SO₂NR^{B8}₂, —SO₂R^{B7}, —SO₂OR^{B7}, —S(═O)R^{B7}, —C(═S)NR^{B8}₂, —C(═O)SR^{B7}, —C(═S)SR^{B7}, —P(═O)₂R^{B7}, —P(═O)(R^{B7})₂, —P(═O)₂NR^{B8}₂, —P(═O)(NR^{B8})₂, —B(OR^{B9})₂, —BR^{B7}(OR^{B9}) and tetrazolyl. In certain embodiments, L is a covalent bond. In certain embodiments, R^{D} is selected from —C(═O)R^{B7}, —CO₂H, —CHO, —CO₂R^{B7}, —C(═O)NR^{B8}₂, —C(═NR^{B8})OR^{B7}, —C(═NR^{B8})NR^{B8}₂, —C(═O)NR^{B8}SO₂R^{B7}, —C(═S)NR^{B8}₂, —C(═O)SR^{B7} and —C(═S)SR^{B7}. In certain embodiments, R^{D} is selected from —C(═O)R^{B7}, —CO₂H, —CHO, and —CO₂R^{B7}. In certain embodiments, R^{D} is —CO₂H.

In certain embodiments, L is a divalent C₁₋₁₀ hydrocarbon chain, wherein one, two or three methylene units of L are optionally and independently replaced with one or more —O—, —S—, —NR^{B8}—, —(C═NR^{B8})—, —C(═O)—, —C(═S)—, —S(═O)—, —S(═O)₂—, divalent C₃₋₁₀ carbocyclyl, divalent 3-14 membered heterocyclyl, divalent C₆₋₁₄ aryl or divalent 5-14 membered heteroaryl group; and R^{D} is selected from —C(═O)R^{B7}, —CO₂H, —CHO, and —CO₂R^{B7}; wherein R^{B7} is selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀carbocyclyl, 3-14 membered heterocyclyl, C₆₋₁₄ aryl, and 5-14 membered heteroaryl.

In certain embodiments, wherein R^{B} is substituted with -L-R^{D}, R^{B} is further substituted with the group —R^{E} wherein R^{E} is selected from halogen, —OH, —OR^{B10}, —ONR^{B11}₂, —NR^{B11}₂, —N(OR^{B12})R^{B12}, —SH, —SR^{B10}, —SSR^{B12}, —OC(═O)R^{B10}, —OCO₂R^{B10}, —OC(═O)NR^{B11}₂, —NR^{B11}C(═O)R^{B1}, —NR^{B11} CO₂R^{B10}, NR^{B11}C(═O)NR^{B11}₂, —OC(═NR^{B11})R^{B10}, —OC(═NR^{B11})OR^{B10}, —OC(═NR^{B11})NR^{B11}₂, NR^{B11}C(═NR^{B11})NR^{B11}₂, —NR^{B11}SO₂R^{B10}, —OSO₂R^{B10}, —OS(═O)R^{B10}, —Si(R^{B10})₃, —OSi(R^{B10})₃, —SC(S)SR^{B10}, —OP(═O)₂R^{B10}, —OP(═O)(R^{B10})₂, —OP(═O)(OR^{B12})₂, —OP(═O)₂NR^{B11}₂, —OP(═O)(NR^{B11})₂, —NR^{B11}P(═O)(OR^{B12})₂, —NR^{B11}P(═O)(NR^{B11})₂, —P(R^{B12})₂, —P(R^{B12})₃, —OP(R^{B12})₂, —OP(R^{B12})₃, 3-14 membered heterocyclyl and 5-14 membered heteroaryl, wherein the point of attachment of the 3-14 membered heterocyclyl or 5-14 membered heteroaryl group is on a nitrogen atom; each R^{B10} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each R^{B11} is, independently, selected from hydrogen, —OH, —OR^{B10}, —NR^{B12}₂, —CN, —C(═O)R^{B10}, —C(═O)NR^{B12}₂, —CO₂R^{B10}, —SO₂R^{B10}, —C(═NR^{B12})OR^{B10}, —C(═NR^{B12})NR^{B12}₂, —SO₂NR^{B12}₂, —SO₂R^{B12}, —SO₂OOR^{B12}, —SOR^{B10}, —C(═S)NR^{B12}₂, —C(═O)SR^{B12}, —C(═S)SR^{B12}, —P(═O)₂R^{B10}, —P(═O)(R^{B10})₂, —P(═O)₂NR^{B12}₂, —P(═O)(NR^{B12})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B11} groups are joined to form a 3-14 membered heterocyclyl or 5-14 membered heteroaryl ring; and each R^{B12} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B12} groups are joined to form a 3-14 membered heterocyclyl or 5-14 membered heteroaryl ring.

In certain embodiments, R^{E} is selected from halogen, —OH, —OR^{B10}, —ONR^{B11}₂, —NR^{B11}₂, —N(OR^{B12})R^{B12}, —SH, —SR^{B10}, —SSR^{B12}, —Si(R^{B10})₃, —OSi(R^{B10})₃, —P(R^{B12})₂, —P(R^{B12})₃, —OP(R^{B12})₂, —OP(R^{B12})₃, 3-14 membered heterocyclyl and 5-14 membered heteroaryl, wherein the point of attachment of the 3-14 membered heterocyclyl or 5-14 membered heteroaryl group is on a nitrogen atom. In certain embodiments, R^{E} is selected from halogen, —OH, —OR^{B10}, —NR^{B11}₂, 3-14 membered heterocyclyl and 5-14 membered heteroaryl, wherein the point of attachment of the 3-14 membered heterocyclyl or 5-14 membered heteroaryl group is on a nitrogen atom. In certain embodiments, R^{E} is selected from halogen, —OR^{B10} and —NR^{B11}₂. In certain embodiments, R^{E} is halogen. In certain embodiments, R^{E} is —OR^{B10}. In certain embodiments, R^{E} is —NR^{B11}₂.

In certain embodiments, -L-R^{D} and —R^{E} are vicinal R^{B} substituents (i.e., attached to two adjacent atoms on the group R^{B}; e.g., ortho to each other). In certain embodiments, -L-R^{D} and —R^{E} are ortho to each other. In certain embodiments, -L-R^{D} and —R^{E} are not vicinal R^{B} substituents (i.e., not attached to two adjacent atoms on the group R^{B}; e.g., meta or para to each other). In certain embodiments, -L-R^{D} and -R^{E}are meta to each other. In certain embodiments, -L-R^{D} and —R^{E} are para to each other.

In certain embodiments, the R^{B} is a group of the formula (vii):

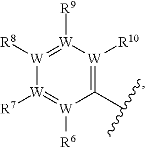

wherein each group W—R⁶, W—R⁷, W—R⁸, W—R⁹, and W—R¹⁰ independently represents either a nitrogen atom (N) or C—R⁶, C—R⁷, C—R⁸, C—R⁹, or C—R¹⁰, respectively; and wherein R⁶, R⁷, R⁸, R⁹ and R¹⁰ are independently selected from hydrogen, halogen, —CN, —NO₂, —N₃, —SO₂H, —SO₃H, —OH, —OR^{B1}, —ONR^{B2}₂, —NR^{B2}₂, —N(OR^{B3})R^{B3}, —SH, —SR^{B1}, —SSR^{B3}, —C(═O)R^{B1}, —CO₂H, —CHO, —C(OR^{B3})₂, —CO₂R^{B1}, —OC(═O)R^{B1}, —OCO₂R^{B1}, —C(═O)NR^{B2}₂, —OC(═O)NR^{B2}₂, —NR^{B2}C(═O)R^{B1}, —NR^{B2}CO₂R^{B1}, —NR^{B2}C(═O)NR^{B2}₂, —C(═NR^{B2})OR^{B1}, —OC(═NR^{B2})R^{B1}, —OC(═NR^{B2})OR^{B1}, —C(═NR^{B2})NR^{B2}₂, —OC(═NR^{B2})NR^{B2}₂, —NR^{B2}C(═NR^{B2})NR^{B2}₂, —C(═O)NR^{B2}SO₂R^{B1}, —NR^{B2}SO₂R^{B1}, —SO₂NR^{B2}₂, —SO₂R^{B1}, —SO₂OR^{B1}, —OSO₂R^{B1}, —S(═O)R^{B1}, —OS(═O)R^{B1}, —Si(R^{B1})₃, —OSi(R^{B1})₃—C(═S)NR^{B2}₂, —C(═O)SR^{B1}, —C(═S)SR^{B1}, —SC(S)SR^{B1}, —P(═O)₂R^{B1}, —OP(═O)₂R^{B1}, —P(═O)(R^{B1})₂, —OP(═O)(R^{B1})₂, —OP(═O)(OR^{B3})₂, —P(═O)₂NR^{B2}₂, —OP(═O)₂NR^{B2}₂, —P(═O)(NR^{B2})₂, —OP(═O)(NR^{B2})₂, —NR^{B2}P(═O)(OR^{B3})₂, —NR^{B2}P(═O)(NR^{B2})₂, —P(R^{B3})₂, —P(R^{B3})₃, —OP(R^{B3})₂, —OP(R^{B3})₃, —B(OR^{B3})₂, or —BR^{B1}(OR^{B3}), C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, 5-14 membered heteroaryl, -L-R^{D} and —R^{E}; or one or more of R⁶ and R⁷, R⁷ and R⁸, R⁸ and R⁹ or R⁹ and R¹⁰ are joined to form a C₃₋₁₀ carbocyclyl, 3-14 membered heterocyclyl, C₆₋₁₄ aryl or 5-14 membered heteroaryl ring; or R¹⁰ and R^{C} are joined to form a 3-14 membered heterocyclyl or 5-14 membered heteroaryl ring; each R^{B1} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each R^{B2} is, independently, selected from hydrogen, —OH, —OR^{B1}, —NR^{B3}₂, —CN, —C(═O)R^{B1}, —C(═O)NR^{B3}₂, —CO₂R^{B1}, —SO₂R^{B1}—C(═NR^{B3})OR^{B1}, —C(═NR^{B3})NR^{B3}₂, —SO₂NR^{B3}₂, —SO₂R^{B3}, —SO₂OR^{B3}, —SOR^{B1}, —C(═S)NR^{B3}₂, —C(═O)SR^{B3}—C(═S)SR^{B3}, —P(═O)₂R^{B1}, —P(═O)(R^{B1})₂, —P(═O)₂NR^{B3}₂, P(═O)(NR^{B3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B2} groups are joined to form a 3-14 membered heterocyclyl or 5-14 membered heteroaryl ring; each R^{B3} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B3} groups are joined to form a 3-14 membered heterocyclyl or 5-14 membered heteroaryl ring; and L, R^{D} and R^{E} are as defined above and herein.

In certain embodiments, the group of formula (vii) represents a 6-14 membered heteroaryl group. In certain embodiments, the group of formula (vii) represents a C₆₋₁₄ aryl group. In certain embodiments, the C₆₋₁₄ aryl group of formula (vii) represents a phenyl group.

As used herein, when one or more of R⁶, R⁷, R⁸, R⁹ and R¹⁰ is referred to as “not hydrogen”, it is meant that one or more of R⁶, R⁷, R⁸, R⁹ and R¹⁰ is independently selected from halogen, —CN, —NO₂, —N₃, —SO₂H, —SO₃H, —OH, —OR^{B1}, —ONR^{B2}₂, —NR^{B2}₂, —N(OR^{B3})R^{B3}, —SH, —SR^{B1}, —SSR^{B3}, —C(═O)R^{B1}, —CO₂H, —CHO, —C(OR^{B3})₂, —CO₂R^{B1}, —OC(═O)R^{B1}, —OCO₂R^{B1}, —C(═O)NR^{B2}₂, —OC(═O)NR^{B2}₂, —NR^{B2}C(═O)R^{B1}, —NR^{B2}CO₂R^{B1}, —NR^{B2}C(═O)NR^{B2}₂, —C(═NR^{B2})OR^{B1}, —OC(═NR^{B2})R^{B1}, —OC(═NR^{B2})OR^{B1}, —C(═NR^{B2})NR^{B2}₂, —OC(═NR^{B2})NR^{B2}₂, —NR^{B2}C(═NR^{B2})NR^{B2}₂, —C(═O)NR^{B2}SO₂R^{B1}, —NR^{B2}SO₂R^{B1}, —SO₂NR^{B2}₂, —SO₂R^{B1}, —SO₂OR^{B1}, —OSO₂R^{B1}, —S(═O)R^{B1}, —OS(═O)R^{B1}, —Si(R^{B1})₃, —OSi(R^{B1})₃—C(═S)NR^{B2}₂, —C(═O)SR^{B1}, —C(═S)SR^{B1}, —SC(═S)SR^{B1}, —P(═O)₂R^{B1}, —OP(═O)₂R^{B1}, —P(═O)(R^{B1})₂, —OP(═O)(R^{B1})₂, —OP(═O)(OR^{B3})₂, —P(═O)₂NR^{B2}₂, —OP(═O)₂NR^{B2}₂, —P(═O)(NR^{B2})₂, —OP(═O)(NR^{B2})₂, —NR^{B2}P(═O)(OR^{B3})₂, —NR^{B2}P(═O)(NR^{B2})₂, —P(R^{B3})₂, —P(R^{B3})₃, —OP(R^{B3})₂, —OP(R^{B3})₃, —B(OR^{B3})₂, or —BR^{B1}(OR^{B3}), C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, -L-R^{D} or —R^{E}; or wherein one or more of R⁶ and R⁷, R⁷ and R⁸, R⁸ and R⁹ or R⁹ and R¹⁰ are joined to form a C₃₋₁₀ carbocyclyl, 3-14 membered heterocyclyl, C₆₋₁₄ aryl or 5-14 membered heteroaryl ring, or wherein R¹⁰ and R^{C} are joined to form a 3-14 membered heterocyclyl or 5-14 membered heteroaryl ring. In certain embodiments, at least one of R⁶, R⁷, R⁸, R⁹, and R¹⁰ is the group -L-R^{D} as defined above and herein. In certain embodiments, at least one of R⁶, R⁷, R⁸, R⁹, and R¹⁰ is the group —R^{E} as defined herein. In certain embodiments, each of R⁶, R⁷, R⁸, R⁹, and R¹⁰ is hydrogen. In certain embodiments, the group of formula (vii) represents a C₆₋₁₄ aryl or a 6-14 membered heteroaryl group. In certain embodiments, the group of formula (vii) represents a 6-14 membered heteroaryl group. In certain embodiments, the group of formula (vii) represents a C₆₋₁₄aryl group. In certain embodiments, the group of formula (vii) represents a phenyl group. In certain embodiments, W—R⁶, W—R⁷, W—R⁸, W—R⁹, and W—R¹⁰ represent C—R⁶, C—R⁷, C—R⁸, C—R⁹, or C—R¹⁰, respectively. For example, in certain embodiments, R^{B} is a group of the formula (viii):

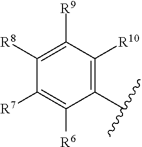

wherein R⁶, R⁷, R⁸, R⁹ and R¹⁰ are as defined above and herein.

In certain embodiments, at least one of R⁶, R⁷, R⁸, R⁹, and R¹⁰ is the group -L-R^{D} as defined above and herein. In certain embodiments, at least one of R⁶, R⁷, R⁸, R⁹, and R¹⁰ is the group —R^{E} as defined herein. In certain embodiments, each of R⁶, R⁷, R⁸, R⁹, and R¹⁰ is hydrogen. In certain embodiments, the group of the formula (viii) represents a C₆₋₁₄ aryl or a 6-14 membered heteroaryl group. In certain embodiments, the group of the formula (viii) represents a 6-14 membered heteroaryl group. In certain embodiments, the group of the formula (viii) represents a C₆₋₁₄ aryl group. In certain embodiments, the C₆₋₁₄ aryl group of the formula (viii) represents a phenyl group.

In certain embodiments, R^{B} is a monosubstituted, disubstituted or trisubstituted group of the formula (viii). In certain embodiments, R^{B} is a monosubstituted or disubstituted group of the formula (viii). In certain embodiments, R^{B} is a monosubstituted group of the formula (viii). For example, in certain embodiments, R^{B} is an ortho-substituted group of formula (viii), e.g., wherein R⁶-R⁹ are hydrogen, and R¹⁰ is not hydrogen. In certain embodiments, R^{B} is a meta-substituted group of the formula (viii), e.g., wherein R⁶-R⁸ and R¹⁰ are hydrogen and R⁹ is not hydrogen. In certain embodiments, R^{B} is a para-substituted group of the formula (viii), e.g., wherein R⁶, R⁷, R⁹ and R¹⁰ are hydrogen and R⁸ is not hydrogen. In certain embodiments, R^{B} is a disubstituted group of the formula (viii). For example, in certain embodiments, R^{B} is a 2,6-disubstituted group of the formula (viii), e.g., wherein R⁷, R⁸ and R⁹ are hydrogen, and R⁶ and R¹⁰ are not hydrogen, e.g., of the formula (viii-d). In certain embodiments, R^{B} is a 2,5-disubstituted group of the formula (viii), e.g., wherein R⁶, R⁸ and R⁹ are hydrogen, and R⁷ and R¹⁰ are not hydrogen. In certain embodiments, R^{B} is a 2,4-disubstituted group of the formula (viii), e.g., wherein R⁶, R⁷ and R⁹ are hydrogen, and R⁸ and R¹⁰ are not hydrogen. In certain embodiments, R^{B} is a 2,3-disubstituted group of formula (viii), e.g., wherein R⁶, R⁷ and R⁸ are hydrogen, and R⁹ and R¹⁰ are not hydrogen. In certain embodiments, R^{B} is a 3,4-disubstituted group of the formula (viii), e.g., wherein R⁶, R⁷ and R¹⁰ are hydrogen, and R⁸ and R⁹ are not hydrogen. In certain embodiments, R^{B} is a 3,5-disubstituted group of the formula (viii), e.g., wherein R⁶, R⁷ and R¹⁰ are hydrogen, and R⁷ and R⁹ are not hydrogen. In certain embodiments, R^{B} is a trisubstituted group of the formula (viii). For example, in certain embodiments, R^{B} is a 2,4,6-trisubstituted group of formula (viii), e.g., wherein R⁷ and R⁹ are hydrogen, and R⁶, R⁸ and R¹⁰ are not hydrogen. In certain embodiments, R^{B} is a 2,3,6-trisubstituted group of the formula (viii), e.g., wherein R² and R³are hydrogen, and R¹, R⁴ and R⁵ are not hydrogen. In certain embodiments, R^{B} is a 2,4,5-trisubstituted group of the formula (viii), e.g., wherein R⁸ and R⁹ are hydrogen, and R⁶, R⁷ and R¹⁰ are not hydrogen. In certain embodiments, R^{B} is a 2,3,4-trisubstituted group of the formula (viii), e.g., wherein R⁶ and R⁹are hydrogen, and R⁷, R⁸ and R¹⁰ are not hydrogen. In certain embodiments, R^{B} is a 3,4,5-trisubstituted group of the formula (viii), e.g., wherein R⁶ and R¹⁰ are hydrogen, and R⁷, R⁸ and R⁹ are not hydrogen. In certain embodiments, R^{B} is heteroaryl selected from a 5-6-membered heteroaryl or a 5,6-bicyclic heteroaryl. In certain embodiments, R^{B} is a 6-membered heteroaryl. In certain embodiments, R^{A} is a 6-membered heteroaryl selected from pyridinyl. In certain embodiments, R^{B} is 2-pyridinyl, 3-pyridinyl or 4-pyridinyl. In certain embodiments, R^{B} is a 2-pyridinyl wherein W—R⁶ is N, and W—R⁷, W—R⁸, W—R⁹, and W—R¹⁰ are C—R⁷, C—R⁸, C—R⁹ and C—R¹⁰, respectively. In certain embodiments, R^{B} is a 3-pyridinyl wherein W—R⁷ is N, and W—R⁶, W—R⁸, W—R⁹, and W—R¹⁰ are C—R⁶, C—R⁸, C—R⁹ and C—R¹⁰, respectively. In certain embodiments, R^{B} is a 4-pyridinyl wherein W—R⁸ is N, and W—R⁶, W—R⁷, W—R⁹, and W—R¹⁰ are C—R⁶, C—R⁷, C—R⁹ and C—R¹⁰, respectively.

In certain embodiments, at least one of R⁶, R⁷, R⁸, R⁹, and R¹⁰ is the group -L-R^{D} as defined above and herein. In certain embodiments, at least one of R⁶, R⁷, R⁸, R⁹, and R¹⁰ is the group —R^{E} as defined herein. In certain embodiments, R^{B} is a monosubstituted or disubstituted pyridinyl. In certain embodiments, R^{B} is a monosubstituted pyridinyl. In certain embodiments, R^{B} is a monosubstituted pyridinyl of the formula (ix) wherein R⁸, R⁹, R¹⁰ are hydrogen and R⁷ is not hydrogen. In certain embodiments, R^{B} is a monosubstituted pyridinyl of the formula (ix) wherein R⁷, R⁹, R¹⁰ are hydrogen and R⁸ is not hydrogen. In certain embodiments, R^{B} is a monosubstituted pyridinyl of the formula (ix) wherein R⁷, R⁸, R¹⁰ are hydrogen and R⁹ is not hydrogen. In certain embodiments, R^{B} is a monosubstituted pyridinyl of the formula (ix) wherein R⁷, R⁸, R⁹ are hydrogen and R¹⁰ is not hydrogen. In certain embodiments, R^{B} is a monosubstituted pyridinyl of the formula (x) wherein R⁸, R⁹, R¹⁰ are hydrogen and R⁶ is not hydrogen. In certain embodiments, R^{B} is a monosubstituted pyridinyl of the formula (x) wherein R⁶, R⁹, R¹⁰ are hydrogen and R⁸ is not hydrogen. In certain embodiments, R^{B} is a monosubstituted pyridinyl of the formula (x) wherein R⁶, R⁸, R¹⁰ are hydrogen and R⁹ is not hydrogen. In certain embodiments, R^{B} is a monosubstituted pyridinyl of the formula (x) wherein R⁶, R⁸, R⁹are hydrogen and R¹⁰ is not hydrogen. In certain embodiments, R^{B} is a monosubstituted pyridinyl of the formula (xi) wherein R⁶, R⁷, R⁹ are hydrogen and R¹⁰ is not hydrogen. In certain embodiments, R^{B} is a monosubstituted pyridinyl of the formula (v) wherein R⁶, R⁷, R¹⁰ are hydrogen and R⁹ is not hydrogen.

In certain embodiments, R^{B} is a disubstituted pyridinyl. In certain embodiments, R^{B} is a disubstituted pyridinyl of the formula (ix) wherein R⁸ and R⁹ are hydrogen and R⁷ and R¹⁰ are not hydrogen. In certain embodiments, R^{B} is a disubstituted pyridinyl of the formula (ix) wherein R⁷ and R⁹ are hydrogen and R⁸ and R¹⁰ are not hydrogen. In certain embodiments, R^{B} is a disubstituted pyridinyl of the formula (ix) wherein R⁷ and R⁸ are hydrogen and R⁹ and R¹⁰ are not hydrogen. In certain embodiments, R^{B} is a disubstituted pyridinyl of the formula (ix) wherein R⁸ and R¹⁰ are hydrogen and R⁷ and R⁹ are not hydrogen. In certain embodiments, R^{B} is a disubstituted pyridinyl of the formula (ix) wherein R⁹ and R¹⁰ are hydrogen and R⁷ and R⁸ are not hydrogen. In certain embodiments, R^{B} is a disubstituted pyridinyl of the formula (ix) wherein R⁷ and R¹⁰ are hydrogen and R⁸ and R⁹ are not hydrogen. In certain embodiments, R^{B} is a disubstituted pyridinyl of the formula (x) wherein R⁸ and R⁹ are hydrogen and R⁶ and R¹⁰ are not hydrogen. In certain embodiments, R^{B} is a disubstituted pyridinyl of the formula (x) wherein R⁸ and R¹⁰ are hydrogen and R⁶ and R⁹ are not hydrogen. In certain embodiments, R^{B} is a disubstituted pyridinyl of the formula (x) wherein R⁹ and R¹⁰ are hydrogen and R⁶ and R⁸ are not hydrogen. In certain embodiments, R^{B} is a disubstituted pyridinyl of the formula (x) wherein R⁶ and R⁹ are hydrogen and R⁸ and R¹⁰ are not hydrogen. In certain embodiments, R^{B} is a disubstituted pyridinyl of the formula (x) wherein R⁶ and R¹⁰ are hydrogen and R⁸ and R⁹ are not hydrogen. In certain embodiments, R^{B} is a disubstituted pyridinyl of the formula (x) wherein R⁶ and R⁸ are hydrogen and R⁹ and R¹⁰ are not hydrogen. In certain embodiments, R^{B} is a disubstituted pyridinyl of the formula (xi) wherein R⁷ and R⁹ are hydrogen and R⁶ and R¹⁰ are not hydrogen. In certain embodiments, R^{B} is a disubstituted pyridinyl of the formula (xi) wherein R⁶ and R⁷ are hydrogen and R⁹ and R¹⁰ are not hydrogen. In certain embodiments, R^{B} is a disubstituted pyridinyl of the formula (xi) wherein R⁶ and R⁸ are hydrogen and R⁷ and R¹⁰ are not hydrogen. In certain embodiments, R^{B} is a disubstituted pyridinyl of the formula (xi) wherein R⁶ and R¹⁰ are hydrogen and R⁷ and R⁹ are not hydrogen.

In certain embodiments, R^{B} is C₅₋₁₀ carbocyclyl or 5-10 membered heterocyclyl of the formula (xii):

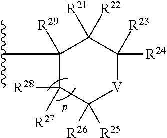

wherein V is N, NR³⁰, O, S or CR³¹R³²; p is 0, 1 or 2; each R²¹, R²², R²³, R²⁴, R²⁵, R²⁶, R²⁷, R²⁸, R²⁹, R³¹ and R³² is independently selected from hydrogen, halogen, —CN, —NO₂, —N₃, —SO₂H, —SO₃H, —OH, —OR^{B1}, —ONR^{B2}₂, —NR^{B2}₂, —N(OR^{B3})R^{B3}, —SH, —SR^{B1}, —SSR^{B3}, —C(═O)R^{B1}, —CO₂H, —CHO, —C(OR^{B3})₂, —CO₂R^{B1}, —OC(═O)R^{B1}, —OCO₂R^{B1}, —C(═O)NR^{B2}₂, —OC(═O)NR^{B2}₂, —NR^{B2}C(═O)R^{B1}, —NR^{B2}CO₂R^{B1}, —NR^{B2}C(═O)NR^{B2}₂, —C(═NR^{B2})OR^{B1}, —OC(═NR^{B2})R^{B1}, —OC(═NR^{B2})OR^{B1}, —C(═NR^{B2})NR^{B2}₂, —OC(═NR^{B2})NR^{B2}₂, —NR^{B2}C(═NR^{B2})NR^{B2}₂, —C(═O)NR^{B2}SO₂R^{B1}, —NR^{B2}SO₂R^{B1}, —SO₂NR^{B2}₂, —SO₂R^{B1}, —SO₂OR^{B1}, —OSO₂R^{B1}, —S(═O)R^{B1}, —OS(═O)R^{B1}, —Si(R^{B1})₃, —OSi(R^{B1})₃, —C(═S)NR^{B2}₂, —C(═O)SR^{B1}, —C(═S)SR^{B1}, —SC(═S)SR^{B1}, —P(═O)₂R^{B1}, —OP(═O)₂R^{B1}, —P(═O)(R^{B1})₂, —OP(═O)(R^{B1})₂, —OP(═O)(OR^{B3})₂, —P(═O)₂NR^{B2}₂, —OP(═O)₂NR^{B2}₂, —P(═O)(NR^{B2})₂, —OP(═O)(NR^{B2})₂, —NR^{B2}P(═O)(OR^{B3})₂, —NR^{B2}P(═O)(NR^{B2})₂, —P(R^{B3})₂, —P(R^{B3})₃, —OP(R^{B3})₂, —OP(R^{B3})₃, —B(OR^{B3})₂, or —BR^{B1}(OR^{B3}), C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, 5-14 membered heteroaryl, -L-R^{D} and —R^{E}; or one or more of R²⁹ and R²¹, R²² and R²³, R²⁴ and R³¹, R³² and R²⁵, R²⁶ and R²⁷, R²⁸ and R²⁹, or R²⁶ and R²⁹, are joined to form a double bond or a C₃₋₁₀ carbocyclyl, 3-14 membered heterocyclyl, C₆₋₁₄ aryl or 5-14 membered heteroaryl ring; optionally wherein X is N, then N and R²³ or N and R²⁵ are joined to form a double bond; R³⁰ is selected from hydrogen, —OH, —OR^{B1}, —NR^{B3}₂, —CN, —C(═O)R^{B1}, —C(═O)NR^{B3}₂, —CO₂R^{B1}, —SO₂R^{B1}, —C(═NR^{B3})OR^{B1}, —C(═NR^{B3})NR^{B3}₂, —SO₂NR^{B3}₂, —SO₂R^{B3}, —SO₂OR^{B3}, —S(═O)R^{B1}, —C(═S)NR^{B3}₂, —C(═O)SR^{B3}, —C(═S)SR^{B3}, —P(═O)₂R^{B1}, —P(═O)(R^{B1})₂, —P(═O)₂NR^{B3}₂, —P(═O)(NR^{B3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, optionally wherein R²⁴ and R³⁰ or R³⁰ and R²⁵ are joined to form a 3-14 membered heterocyclyl or 5-14 membered heteroaryl ring; wherein: each R^{B1} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each R^{B2} is, independently, selected from hydrogen, —OH, —OR^{B1}, —NR^{B3}₂, —CN, —C(═O)R^{B1}, —C(═O)NR^{B3}₂, —CO₂R^{B1}, —SO₂R^{B1}, —C(═NR^{B3})OR^{B1}, —C(═NR^{B3})NR^{B3}₂, —SO₂NR^{B3}₂, —SO₂R^{B3}, —SO₂OR^{B3}, —SOR^{B1}, —C(═S)NR^{B3}₂, —C(═O)SR^{B3}, —C(═S)SR^{B3}, —P(═O)₂R^{B1}, —P(═O)(R^{B1})₂, —P(═O)₂NR^{B3}₂, —P(═O)(NR^{B3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B2} groups are joined to form a 3-14 membered heterocyclyl or 5-14 membered heteroaryl ring; each R^{B3} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B3} groups are joined to form a 3-14 membered heterocyclyl or 5-14 membered heteroaryl ring; and L, R^{D} and R^{E} are as defined above and herein.

In certain embodiments, at least one of R²¹, R²², R²³, R²⁴, R²⁵, R²⁶, R²⁷, R²⁸, R²⁹, R³⁰, R³¹, and R³² is the group -L-R^{D} as defined above and herein. In certain embodiments, at least one of R²¹, R²², R²³, R²⁴, R²⁵, R²⁶, R²⁷, R²⁸, R²⁹, R³⁰, R³¹, and R³² is selected from the group —R^{E} as defined herein. In certain embodiments, p is 0. In certain embodiments, p is 1. In certain embodiments, p is 2. In certain embodiments, V is N. In certain embodiments, V is NR³⁰. In certain embodiments, V is O. In certain embodiments, V is S. In certain embodiments, V is CR³¹R³². In certain embodiments, R^{B} is 05-10 carbocyclyl or 5-10 membered heterocyclyl of the formula (xiii):

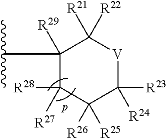

wherein: V is N, NR³⁰, O, S or CR³¹R³²; p is 0, 1 or 2; each R²¹, R²², R²³, R²⁴, R²⁵, R²⁶, R²⁷, R²⁸, R²⁹, R³¹ and R³² is independently selected from hydrogen, halogen, —CN, —NO₂, —N₃, —SO₂H, —SO₃H, —OH, —OR^{B1}, —ONR^{B2}₂, —NR^{B2}₂, —N(OR^{B3})R^{B3}, —SH, —SR^{B1}, —SSR^{B3}, —C(═O)R^{B1}, —CO₂H, —CHO, —C(OR^{B3})₂, —CO₂R^{B1}, —OC(═O)R^{B1}, —OCO₂R^{B1}, —C(═O)NR^{B2}₂, —OC(═O)NR^{B2}₂, —NR^{B2}C(═O)R^{B1}, —NR^{B2}CO₂R^{B1}, —NR^{B2}C(═O)NR^{B2}₂, —C(═NR^{B2})OR^{B1}, —OC(═NR^{B2})R^{B1}, —OC(═NR^{B2})OR^{B1}, —C(═NR^{B2})NR^{B2}₂, —OC(═NR^{B2})NR^{B2}₂, —NR^{B2}C(═NR^{B2})NR^{B2}₂, —C(═O)NR^{B2}SO₂R^{B1}, —NR^{B2}SO₂R^{B1}, —SO₂NR^{B2}₂, —SO₂R^{B1}, —SO₂OR^{B1}, —OSO₂R^{B1}, —S(═O)R^{B1}, —OS(═O)R^{B1}, —Si(R^{B1})₃, —OSi(R^{B1})₃—C(═S)NR^{B2}₂, —C(═O)SR^{B1}, —C(═S)SR^{B1}, —SC(═S)SR^{B1}, —P(═O)₂R^{B1}, —OP(═O)₂R^{B1}, —P(═O)(R^{B1})₂, —OP(═O)(R^{B1})₂, —OP(═O)(OR^{B3})₂, —P(═O)₂NR^{B2}₂, —OP(═O)₂NR^{B2}₂, —P(═O)(NR^{B2})₂, —OP(═O)(NR^{B2})₂, —NR^{B2}P(═O)(OR^{B3})₂, NR^{B2}P(═O)(NR^{B2})₂, —P(R^{B3})₂, —P(R^{B3})₃, —OP(R^{B3})₂, —OP(R^{B3})₃, —B(OR^{B3})₂, or —BR^{B1}(OR^{B3}), C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, 5-14 membered heteroaryl, -L-R^{D} and —R^{E}; or one or more of R²⁹ and R²¹, R²² and R³¹, R³² and R²³, R²⁴ and R²⁵, R²⁶ and R²⁷, R²⁸ and R²⁹, and R²⁶ and R²⁹, are joined to form a double bond or a C₃₋₁₀ carbocyclyl, 3-14 membered heterocyclyl, C₆₋₁₄ aryl or 5-14 membered heteroaryl ring; optionally wherein X is N, then N and R²¹ or N and R²³ are joined to form a double bond; R³⁰ is selected from hydrogen, —OH, —OR^{B1}, —NR^{B3}₂, —CN, —C(═O)R^{B1}, —C(═O)NR^{B3}₂, —CO₂R^{B1}, —SO₂R^{B1}, —C(═NR^{B3})OR^{B1}, —C(═NR^{B3})NR^{B3}₂, —SO₂NR^{B3}₂, —SO₂R^{B3}, —SO₂OR^{B3}, —S(═O)R^{B1}, —C(═S)NR^{B3}₂, —C(═O)SR^{B3}, —C(═S)SR^{B3}, —P(═O)₂R^{B1}, —P(═O)(R^{B1})₂, —P(═O)₂NR^{B3}₂, —P(═O)(NR^{B3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or R²² and R³⁰ or R³⁰ and R²³ are joined to form a 3-14 membered heterocyclyl or 5-14 membered heteroaryl ring; wherein: each R^{B1} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl; each R^{B2} is, independently, selected from hydrogen, —OH, —OR^{B1}, —NR^{B3}₂, —CN, —C(═O)R^{B1}, —C(═O)NR^{B3}₂, —CO₂R^{B1}, —SO₂R^{B1}, —C(═NR^{B3})OR^{B1}, —C(═NR^{B3})NR^{B3}₂, —SO₂NR^{B3}₂, —SO₂R^{B3}, —SO₂OR^{B3}, —S(═O)R^{B1}, —C(═S)NR^{B3}₂, —C(═O)SR^{B3}, —C(═S)SR^{B3}, —P(═O)₂R^{B1}, —O(═O)(R^{B1})₂, P(═O)₂NR^{B3}₂, —P(═O)(NR^{B3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B2} groups are joined to form a 3-14 membered heterocyclyl or 5-14 membered heteroaryl ring; each R^{B3} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B3} groups are joined to form a 3-14 membered heterocyclyl or 5-14 membered heteroaryl ring; and L, R^{D} and R^{E} are as defined above and herein.

In certain embodiments, at least one of R²¹, R²², R²³, R²⁴, R²⁵, R²⁶, R²⁷, R²⁸, R²⁹, R³⁰, R³¹, and R³² is the group -L-R^{D} as defined above and herein. In certain embodiments, at least one of at least one of R²¹, R²², R²³, R²⁴, R²⁵, R²⁶, R²⁷, R²⁸, R²⁹, R³⁰, R³¹, and R³² is selected from —R^{E} as defined herein. In certain embodiments, p is 0. In certain embodiments, p is 1. In certain embodiments, p is 2. In certain embodiments, V is N. In certain embodiments, V is NR³⁰. In certain embodiments, V is O. In certain embodiments, V is S. In certain embodiments, V is CR³¹R³². In certain embodiments, X is NR³⁰. In certain embodiments, V is CR³¹, R³².

As described generally above, in certain embodiments, R^{B} and R^{C} together with the nitrogen (N) atom to which each is attached are joined to form a 5-14 membered carbocyclyl, heterocyclyl, aryl or heteroaryl ring.

For example, in certain embodiments, R^{B} and R^{C} together with the nitrogen (N) atom to which each is attached are joined to form a 5-14 membered ring of the formula (xiv):

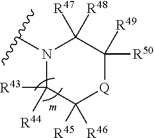

wherein: Q is N, NR⁴⁰, O, S, or CR⁴¹R⁴², M is 0, 1 or 2; and each R⁴¹, R⁴², R⁴³, R⁴⁴, R⁴⁵, R⁴⁶, R⁴⁷, R⁴⁸, R⁴⁹ and R⁵⁰ is independently selected from hydrogen, halogen, —CN, —NO₂, —N₃, —SO₂H, —SO₃H, —OH, —OR^{F1}, —ONR^{F2}₂, —NR^{F2}₂, —N(OR^{F3})R^{F3}, —SH, —SR^{F1}, —SSR^{F3}, —C(═O)R^{F1}, —CO₂H, —CHO, —C(OR^{F3})₂, —CO₂R^{F1}, OC(═O)R^{F1}, —OCO₂R^{F1}, —C(═O)NR^{F2}₂, —OC(═O)NR^{F2}₂, —NR^{F2}C(═O)R^{F1}, —NR^{F2}CO₂R^{F1}, —NR^{F2}C(═O)NR^{F2}₂, —C(═NR^{F2})OR^{F1}, —OC(═NR^{F2})R^{F1}, —OC(═NR^{F2})OR^{F1}, —C(═NR^{F2})NR^{F2}₂, —OC(═NR^{F2})NR^{F2}₂, —NR^{F2}C(═NR^{F2})NR^{F2}₂, —C(═O)NR^{F2}SO₂R^{BC1}, —NR^{F2}SO₂R^{F1}, —SO₂NR^{F2}₂, —SO₂R^{F1}, —SO₂OR^{F1}, —OSO₂R^{F1}, —S(═O)R^{F1}, —OS(═O)R^{F1}, —Si(R^{F1})₃, —OSi(R^{F1})₃—C(═S)NR^{F2}₂, —C(═O)SR^{F1}, —C(═S)SR^{F1}, —SC(═S)SR^{F1}, P(═O)₂R^{F1}, —OP(═O)₂R^{F1}, —P(═O)(R^{F1})₂, —OP(═O)(R^{F1})₂, —OP(═O)(OR^{F3})₂, —P(═O)₂NR^{F2}₂, —OP(═O)₂NR^{F2}₂, —P(═O)(NR^{F2})₂, —OP(═O)(NR^{F2})₂, —NR^{F2}P(═O)(OR^{F3})₂, —NR^{F2}P(═O)(NR^{F2})₂, —P(R^{F3})₂, —P(R^{F3})₃, —OP(R^{F3})₂, —OP(R^{F3})₃, —B(OR^{F3})₂, or —BR^{F1}(OR^{F3}), C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, 5-14 membered heteroaryl, -L-R^{D} and —R^{E}; or one or more of R⁴⁷ and R⁴⁹, R⁴⁸ and R⁵⁰, R⁴⁹ and R⁴¹, R⁵⁰ and R⁴², R⁴¹ and R⁴⁵, R⁴² and R⁴⁶, R⁴⁵ and R⁴³, and R⁴⁶ and R⁴⁴, are joined to form a double bond or a C₃₋₁₀ carbocyclyl, 3-14 membered heterocyclyl, C₆₋₁₄ aryl or 5-14 membered heteroaryl ring; optionally wherein Q is N, then N and R⁴⁹ or N and R⁴⁶ are joined to form a double bond; R⁴⁰ is selected from hydrogen, —OH, —OR^{F1}, —NR^{F3}₂, —CN, —C(═O)R^{F1}, —C(═O)NR^{F3}₂, —CO₂R^{F1}, —SO₂R^{F1}, —C(═NR^{F3})OR^{F1}, —C(═NR^{F3})NR^{F3}₂, —SO₂NR^{F3}₂, —SO₂R^{F3}, —SO₂OR^{F3}, —SOR^{F1}, —C(═S)NR^{F3}₂, —C(═O)SR^{F3}, —C(═S)SR^{F3}, —P(═O)₂R^{F1}, —P(═O)(R^{F1})₂, —P(═O)₂NR^{F3}₂, —P(═O)(NR^{F3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, optionally wherein R⁴⁹ and R⁴⁰ or R⁴⁰ and R⁴⁵ are joined to form a 3-14 membered heterocyclyl, or 5-14 membered heteroaryl ring; each R^{F1} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl; each R^{F2} is, independently, selected from hydrogen, —OH, —OR^{F1}, —NR^{F3}₂, —CN, —C(═O)R^{F1}, —C(═O)NR^{F3}₂, —CO₂R^{F1}, —SO₂R^{F1}, —C(—NR^{F3})OR^{F1}, —C(—NR^{F3})NR^{F3}₂, —SO₂NR^{F3}₂, —SO₂R^{F3}, —SO₂OR^{F3}, —S(═O)R^{F1}, —C(═S)NR^{F3}₂, —C(═O)SR^{F3}, —C(═S)SR^{F3}, —P(═O)₂R^{F1}, —P(═O)(R^{F1})₂, —P(═O)₂NR^{F3}₂, —P(═O)(NR^{F3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{F2} groups are joined to form a 3-14 membered heterocyclyl or 5-14 membered heteroaryl ring; each R^{F3} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{F3} groups are joined to form a 3-14 membered heterocyclyl or 5-14 membered heteroaryl ring; and L, R^{D} and R^{E} are as defined above and herein.

In certain embodiments, at least one of R⁴⁰, R⁴¹, R⁴², R⁴³, R⁴⁴, R⁴⁵, R⁴⁶, R⁴⁷, R⁴⁸, R⁴⁹ and R⁵⁰ is the group -L-R^{D} as defined above and herein. In certain embodiments, at least one of R⁴⁰, R⁴¹, R⁴², R⁴³, R⁴⁴, R⁴⁵, R⁴⁶, R⁴⁷, R⁴⁸, R⁴⁹ and R⁵⁰ is selected from —R^{E} as defined herein. In certain embodiments, each of R⁴⁰, R⁴¹, R⁴², R⁴³, R⁴⁴, R⁴⁵, R⁴⁶, R⁴⁷, R⁴⁸, R⁴⁹ and R⁵⁰ is hydrogen.

In certain embodiments, m is 0. In certain embodiments, m is 1. In certain embodiments, m is 2. In certain embodiments, Q is N. In certain embodiments, Q is NR⁴⁰. In certain embodiments, Q is O. In certain embodiments, Q is S. In certain embodiments, Q is CR⁴¹R⁴². In certain embodiments, R⁴⁷ and R⁴⁹ or R⁴⁸ and R⁵⁰ are joined to form a C₃₋₁₀ carbocyclyl, 3-14 membered heterocyclyl, C₆₋₁₄ aryl or 5-14 membered heteroaryl ring.

For example, in certain embodiments, R⁴⁷ and R⁴⁹ are joined to form a C₃₋₁₀ carbocyclyl and Q is CR⁴¹R⁴². In certain embodiments, each R⁴¹, R⁴², R⁴³, R⁴⁴, R⁴⁵, R⁴⁶, R⁴⁸, R⁵⁰ are hydrogen; and R⁴⁷ and R⁴⁹ are joined to form a C₃₋₁₀ carbocyclyl. In certain embodiments, m is 1. In certain embodiments, R^{B} and R^{C} together with the nitrogen (N) atom to which each is attached are joined to form

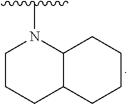 [SMILES: *N1CCCC2CCCCC21]

In certain embodiments, R⁴⁷ and R⁴⁹ are joined to form a double bond and R⁴⁸ and R⁵⁰ are joined to form a C₆₋₁₄ aryl or 5-14 membered heteroaryl. For example, in certain embodiments, R^{B} and R^{C} together with the nitrogen (N) atom to which each is attached are joined to form a 5-14 membered ring of the formula (xv):

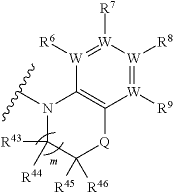

wherein Q, m, R⁴¹, R⁴², R⁴³, R⁴⁴, R⁴⁵, R⁴⁶, R⁶, R⁷, R⁸ and R⁹ are as defined above and herein. In certain embodiments, Q is CR⁴¹R⁴², R⁴⁹ and R⁴¹ are joined to form a double bond and R⁵⁰ and R⁴² are joined to form a C₆₋₁₄ aryl or 5-14 membered heteroaryl. For example, in certain embodiments, R^{B} and R^{C} together with the nitrogen (N) atom to which each is attached are joined to form a group of the formula (xvi):

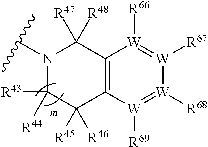

wherein m, R⁴³, R⁴⁴, R⁴⁵, R⁴⁶, R⁴⁷ and R⁴⁸ are as defined above and herein; and wherein R⁶⁶, R⁶⁷, R⁶⁸ and R⁶⁹ are independently selected from hydrogen, halogen, —CN, —NO₂, —N₃, —SO₂H, —SO₃H, —OH, —OR^{F4}, —ONR^{F5}₂, —NR^{F5}₂, —N(OR^{F6})R^{F6}, —SH, —SR^{E4}, —SSR^{F6}, —C(═O)R^{E4}, —CO₂H, —CHO, —C(OR^{F6})₂, —CO₂R^{F4}, —OC(═O)R^{F4}, —OCO₂R^{F4}, —C(═O)NR^{F5}₂, —OC(═O)NR^{F5}₂, —NR^{F5}C(═O)R^{F4}, —NR^{F5}CO₂R^{F4}, —NR^{F5}C(═O)NR^{F5}₂, —C(═NR^{F5})OR^{F4}, —OC(═NR^{F5})R^{F4}, —OC(═NR^{F5})OR^{F4}, —C(═NR^{F5})NR^{F5}₂, —OC(═NR^{F5})NR^{F5}₂, —NR^{F5}C(═NR^{F5})NR^{F5}₂, —C(═O)NR^{F5}SO₂R^{F4}, —NR^{F5}SO₂R^{F4}, —SO₂NR^{F5}₂, —SO₂R^{F4}, —SO₂OR^{F4}, —OSO₂R^{F4}, —S(═O)R^{F4}, —OS(═O)R^{F4}, —Si(R^{F4})₃, —OSi(R^{F4})₃—C(═S)NR^{F5}₂, —C(═O)SR^{F4}, —C(═S)SR^{F4}, —SC(S)SR^{F4}, —P(═O)₂R^{F4}, —OP(═O)₂R^{F4}, —P(═O)(R^{F4})₂, —OP(═O)(R^{F4})₂, —OP(═O)(OR^{F6})₂, P(═O)₂NR^{F5}₂, —OP(═O)₂NR^{F5}₂, —P(═O)(NR^{F5})₂, —OP(═O)(NR^{F5})₂, —NR^{F5}P(═O)(OR^{F6})₂, NR^{F5}P(═O)(NR^{F5})₂, —P(R^{F6})₂, —P(R^{F6})₃, —OP(R^{F6})₂, —OP(R^{F6})₃, —B(OR^{F6})₂, or —BR^{F4}(OR^{F6}), C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, 5-14 membered heteroaryl, -L-R^{D} and —R^{E}; or one or more of R⁶⁶ and R⁶⁷, R⁶⁷ and R⁶⁸, and R⁶⁸ and R⁶⁹ are joined to form a C₃₋₁₀ carbocyclyl, 3-14 membered heterocyclyl, C₆₋₁₄ aryl or 5-14 membered heteroaryl ring; each R^{F4} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each R^{F5} is, independently, selected from hydrogen, —OH, —OR^{F4}, —NR^{F6}₂, —CN, —C(═O)R^{F4}, —C(═O)NR^{F6}₂, —CO₂R^{F4}, —SO₂R^{F4}, —C(═NR^{F6})OR^{F4}, —C(═NR^{F6})NR^{F6}₂, —SO₂NR^{F6}₂, —SO₂R^{F6}, —SO₂OR^{F6}, —SOR^{F4}, —C(═S)NR^{F6}₂, —C(═O)SR^{F6}, —C(═S)SR^{F6}, —P(═O)₂R^{F4}, —P(═O)(R^{F4})₂, —P(═O)₂NR^{F6}₂, —P(═O)(NR^{F6})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{F5} groups are joined to form a 3-14 membered heterocyclyl or 5-14 membered heteroaryl ring; and each R^{F6} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{F6} groups are joined to form a 3-14 membered heterocyclyl or 5-14 membered heteroaryl ring.

In certain embodiments, at least one of R⁴³, R⁴⁴, R⁴⁵, R⁴⁶, R⁴⁷, R⁴⁸, R⁶⁶, R⁶⁷, R⁶⁸ and R⁶⁹ is the group -L-R^{D} as defined above and herein. In certain embodiments, at least one of R⁴³, R⁴⁴, R⁴⁵, R⁴⁶, R⁴⁷, R⁴⁸, R⁶⁶, R⁶⁷, R⁶⁸ and R⁶⁹ is selected from —R^{E} as defined herein. In certain embodiments, m is 0. In certain embodiments, m is 1. In certain embodiments, m is 2.

As described generally above, R^{C} is selected from hydrogen, —OH, —OR^{C1}, —ONR^{C2}₂, —NR^{C2}₂, —C(═O)R^{C1}, —CHO, —CO₂R^{C1}, —C(═O)NR^{C2}₂, —C(═NR^{C2})OR^{C1}, —C(═NR^{C2})NR^{C2}₂, —SO₂R^{C1}, —S(═O)R^{C1}, —Si(R^{C1})₃, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; wherein: each R^{C1} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; and each R^{C2} is, independently, selected from hydrogen, —OH, —OR^{C1}, —NR^{C3}₂, —CN, —C(═O)R^{C1}, —C(═O)NR^{C3}₂, —CO₂R^{C1}, —SO₂R^{C1}, —C(═NR^{C3})OR^{C1}, C(═NR^{C3})NR^{C3}₂, —SO₂NR^{C3}₂, —SO₂R^{C3}, —SO₂OR³, —SOR^{C1}, —C(═S)NR^{C3}₂, —C(═O)SR^{C3}, —C(═S)SR^{C3}, —P(═O)₂R^{C1}, —P(═O)(R^{C1})₂, —P(═O)₂NR^{C3}₂, —P(═O)(NR^{C3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{C2} groups are joined to form a 3-14 membered heterocyclyl or 5-14 membered heteroaryl ring; each R^{C3} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; or R^{B} and R^{C} together with the nitrogen (N) atom to which each is attached are joined to form a 5-14 membered carbocyclyl, heterocyclyl, aryl or heteroaryl ring.

In certain embodiments, each R^{C1} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl. In certain embodiments, each R^{C2} is, independently, selected from hydrogen, —OH, —OR^{C1}, —NR^{C3}₂, —CN, —C(═O)R^{C1}, —C(═NR^{C3}₂, —CO₂R¹, —SO₂R¹, —C(═NR^{C3})OR¹, —C(═NR^{C3})NR^{C3}₂, —SO₂NR^{C3}₂, —SO₂R^{C3}, —SO₂OR³, —SOR^{C1}, —C(═S)NR^{C3}₂, —C(═O)SR^{C3}, —C(═S)SR^{C3}, —P(═O)₂R¹, —P(═O)(R^{C1})₂, —P(═O)₂NR^{C3}₂, —P(═O)(NR^{C3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl. However, in certain embodiments, R^{C} is not any one of hydrogen, —OH, —OR^{C1}, —ONR^{C2}₂, —NR^{C2}₂, —C(═O)R^{C1}, —CHO, —CO₂R^{C1}, —C(═O)NR^{C2}₂, —C(═NR^{C2})OR^{C1}, —C(═NR^{C2})NR^{C2}₂, —SO₂R^{C1}, —S(═O)R^{C1}, or —Si(R^{C1})₃. In certain embodiments, R^{C} is selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl. In certain embodiments, R^{C} is selected from an unsubstituted group, e.g., for example, selected from unsubstituted C₁₋₁₀ alkyl, unsubstituted C₂₋₁₀ alkenyl, unsubstituted C₂₋₁₀ alkynyl, unsubstituted 3-14 membered heteroaliphatic, unsubstituted C₃₋₁₀ carbocyclyl, unsubstituted 3-14 membered heterocyclyl, unsubstituted C₆₋₁₄ aryl and unsubstituted 5-14 membered heteroaryl. However, in certain embodiments, R^{C} is an unsubstituted group wherein —CH₃ and —CH₂CH₃ are excluded.

In certain embodiments, R^{C} is a group having 2 or more carbon atoms, e.g., for example, selected from C₂₋₁₀ alkyl, C₂₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl. In certain embodiments, R^{C} is an unsubstituted group having 2 or more carbon atoms. However, in certain embodiments, R^{C} is a group having 2 or more carbon atoms wherein —CH₂CH₃ is excluded.

In certain embodiments, R^{C} is a group having 3 or more carbon atoms, e.g., for example, selected from C₃₋₁₀ alkyl, C₃₋₁₀ perhaloalkyl, C₃₋₁₀ alkenyl, C₃₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl. In certain embodiments, R^{C} is an unsubstituted group having 3 or more carbon atoms. However, in certain embodiments, R^{C} is a group having 3 or more carbon atoms wherein —CH(CH₃)₂ is excluded.

In certain embodiments, R^{C} is a group having 4 or more carbon atoms, e.g., for example, selected from C₄₋₁₀ alkyl, C₄₋₁₀ perhaloalkyl, C₄₋₁₀ alkenyl, C₄₋₁₀ alkynyl, 5-14 membered heteroaliphatic, C₅₋₁₀ carbocyclyl, 5-14 membered heterocyclyl, C₆₋₁₄ aryl, and 5-14 membered heteroaryl. In certain embodiments, R^{C} is an unsubstituted group having 4 or more carbon atoms.

In certain embodiments, R^{C} is an acyclic group, e.g., for example, selected from C₁₋₁₀ alkyl, C₂₋₁₀ alkenyl, C₂₋₁₀ alkynyl and 3-14 membered heteroaliphatic. In certain embodiments, R^{C} is an unsubstituted acyclic group, e.g., for example, selected from unsubstituted C₁₋₁₀ alkyl, unsubstituted C₂₋₁₀ alkenyl, unsubstituted C₂₋₁₀ alkynyl and unsubstituted 3-14 membered heteroaliphatic. However, in certain embodiments, R^{C} is an acyclic group wherein —CH₃ and —CH₂CH₃ are excluded.

In certain embodiments, R^{C} is C₁₋₁₀ alkyl. In certain embodiments, R^{C} is an unsubstituted C₁₋₁₀ alkyl. In certain embodiments, R^{C} is C₁₋₁₀ alkyl, wherein —CH₃ is excluded. In certain embodiments, R^{C} is C₁₋₁₀ alkyl, wherein —CH₂CH₃ is excluded. In certain embodiments, R^{C} is C₁₋₁₀ alkyl, wherein —CH(CH₃)₂ is excluded. In some embodiments, R^{C} is unsubstituted ethyl or unsubstituted isopropyl.

In certain embodiments, R^{C} is C₂₋₁₀ alkyl, e.g., for example, selected from ethyl, n-propyl, isopropyl, n-butyl, tert-butyl, sec-butyl, iso-butyl, n-pentyl, pentan-3-yl, amyl, neopentyl, 3-methyl-2-butanyl, tertiary amyl and n-hexyl. In certain embodiments, R^{C} is an unsubstituted C₂₋₁₀ alkyl. In certain embodiments, R^{C} is C₂₋₁₀ alkyl, wherein —CH₂CH₃ is excluded. In certain embodiments, R^{C} is C₂₋₁₀ alkyl, wherein —CH(CH₃)₂ is excluded.

In certain embodiments, R^{C} is C₃₋₁₀ alkyl, e.g., for example, selected from n-propyl, isopropyl, n-butyl, tert-butyl, sec-butyl, iso-butyl, n-pentyl, pentan-3-yl, amyl, neopentyl, 3-methyl-2-butanyl, tertiary amyl and n-hexyl. In certain embodiments, R^{C} is an unsubstituted C₃₋₁₀ alkyl. In certain embodiments, R^{C} is C₃₋₁₀ alkyl, wherein —CH(CH₃)₂ is excluded.

In certain embodiments, R^{C} is C₄₋₁₀ alkyl, e.g., for example, selected from n-butyl, tert-butyl, sec-butyl, iso-butyl, n-pentyl, pentan-3-yl, amyl, neopentyl, 3-methyl-2-butanyl, tertiary amyl and n-hexyl. In certain embodiments, R^{C} is an unsubstituted C₄₋₁₀ alkyl.

In certain embodiments, R^{C} is C₂₋₁₀ alkenyl. In certain embodiments, R^{C} is an unsubstituted C₂₋₁₀ alkenyl. In certain embodiments, R^{C} is C₂₋₁₀ alkenyl selected from allyl. In certain embodiments, R^{C} is C₂₋₁₀ alkynyl. In certain embodiments, R^{C} is an unsubstituted C₂₋₁₀ alkynyl. In certain embodiments, R^{C} is 3-14 membered heteroaliphatic. In certain embodiments, R^{C} is an unsubstituted 3-14 membered heteroaliphatic. In certain embodiments, R^{C} is a cyclic group, e.g., selected from C₃₋₁₀ carbocyclyl, 3-14 membered heterocyclyl, C₆₋₁₄ aryl and 5-14 membered heteroaryl. In certain embodiments, R^{C} is an unsubstituted cyclic group, e.g., selected from unsubstituted C₃₋₁₀ carbocyclyl, unsubstituted 3-14 membered heterocyclyl, unsubstituted C₆₋₁₄ aryl and unsubstituted 5-14 membered heteroaryl. In certain embodiments, R^{C} is C₃₋₁₀ carbocyclyl. In certain embodiments, R^{C} is C₄₋₁₀ carbocyclyl. In certain embodiments, R^{C} is C₃₋₁₀ carbocyclyl. In certain embodiments, R^{C} is C₅₋₈ carbocyclyl. In certain embodiments, R^{C} is C₃₋₁₀ carbocyclyl selected from cyclopropyl (C₃), cyclobutyl (C₄), cyclopentyl (C₅), cyclopentenyl (C₅), cyclohexyl (C₆), cyclohexenyl (C₆), cyclohexadienyl (C₆), cycloheptyl (C₇), cycloheptadienyl (C₇), cycloheptatrienyl (C₇) and cyclooctyl (C₈). In certain embodiments, R^{C} is C₃₋₁₀ carbocyclyl selected from cyclopentyl and cyclohexyl. In certain embodiments, R^{C} is an unsubstituted C₃₋₁₀ carbocyclyl.

In certain embodiments, R^{C} is 3-14 membered heterocyclyl. In certain embodiments, R^{C} is 5-10 membered heterocyclyl. In certain embodiments, R^{C} is 5-6 membered heterocyclyl. In certain embodiments, R^{C} is 3-14 membered heterocyclyl selected from azirdinyl, oxiranyl, thiorenyl, azetidinyl, oxetanyl, thietanyl, tetrahydrofuranyl, dihydrofuranyl, tetrahydrothiophenyl, dihydrothiophenyl, pyrrolidinyl, dihydropyrrolyl, dioxolanyl, oxathiolanyl, dithiolanyl, piperidinyl, tetrahydropyranyl, dihydropyridinyl, thianyl, piperazinyl, morpholinyl, dithianyl, dioxanyl, azepanyl, oxepanyl thiepanyl, azocanyl, oxecanyl and thiocanyl. In certain embodiments, R^{C} is 3-14 membered heterocyclyl selected from tetrahydropyranyl. In certain embodiments, R^{C} is an unsubstituted 3-14 membered heterocyclyl.

In certain embodiments, R^{C} is C₆₋₁₄ aryl. In certain embodiments, R^{C} is a C₆₋₁₄ aryl selected from phenyl, naphthyl and anthracyl. In certain embodiments, R^{C} a C₆₋₁₄ aryl selected from phenyl. In certain embodiments, R^{C} is an unsubstituted C₆₋₁₄ aryl.

In certain embodiments, R^{C} is 5-14 membered heteroaryl. In certain embodiments, R^{C} is 5-10 membered heteroaryl. In certain embodiments, R^{C} is 5-6 membered heteroaryl. In certain embodiments, R^{C} is a 5-membered heteroaryl, e.g., for example, selected from pyrrolyl, furanyl, thiophenyl, imidazolyl, pyrazolyl, oxazolyl, isoxazolyl, thiazolyl, isothiazolyl, triazolyl, oxadiazolyl, thiadiazolyl and tetrazolyl. In certain embodiments, R^{A} is a 6-membered heteroaryl, e.g., for example, selected from pyridinyl, pyridazinyl, pyrimidinyl, pyrazinyl, triazinyl and tetrazinyl. In certain embodiments, R^{C} is an unsubstituted 5-14 membered heteroaryl.

In certain embodiments, X is —CN.

For example, in certain embodiments, both R^{B} and R^{C} are cyclic, i.e., R^{B} is selected from C₃₋₁₀ carbocyclyl, 3-14 membered heterocyclyl, C₆₋₁₄ aryl and 5-14 membered heteroaryl, and R^{C} is selected from C₃₋₁₀ carbocyclyl, 3-14 membered heterocyclyl, C₆₋₁₄ aryl, and 5-14 membered heteroaryl. In certain embodiments, R^{C} is a group having 2 or more carbon atoms. In certain embodiments, R^{C} is a group having 3 or more carbon atoms. In certain embodiments, R^{C} is a group having 4 or more carbon atoms. In certain embodiments, R^{C} is an unsubstituted cyclic group.

In certain embodiments, R^{B} is cyclic and R^{C} is acyclic, i.e., R^{B} is selected from C₃₋₁₀ carbocyclyl, 3-14 membered heterocyclyl, C₆₋₁₄ aryl and 5-14 membered heteroaryl and R^{C} is selected from —OH, —OR^{C1}, —ONR^{C2}₂, —NR^{C2}₂, —C(═O)R^{C1}, —CHO, —CO₂R^{C1}, —C(═O)NR^{C2}₂, —C(═NR^{C2})OR^{C1}, —C(═NR^{C2})NR^{C2}₂, —SO₂R^{C1}, —S(═O)R^{C1}, —Si(R^{C1})₃, C₁₋₁₀ alkyl, C₁₋₁₀ perhaloalkyl, C₂₋₁₀ alkenyl, C₂₋₁₀ alkynyl, 3-14 membered heteroaliphatic. In certain embodiments, R^{C} is an acyclic group having 2 or more carbon atoms. In certain embodiments, R^{C} is an acyclic group having 3 or more carbon atoms. In certain embodiments, R^{C} is an acyclic group having 4 or more carbon atoms. In certain embodiments, R^{C} is an unsubstituted acyclic group. For example, R^{B} is C₆₋₁₄ aryl or 5-14 membered heteroaryl; and R^{C} is C₁₋₁₀ alkyl, e.g., R^{B} is C₆₋₁₄ aryl; and R^{C} is C₁₋₁₀ alkyl.

In certain embodiments, R^{A} and R^{B} are independently selected from C₆₋₁₄ aryl and 5-14 membered heteroaryl. In certain embodiments, R^{A} is C₆₋₁₄ aryl and R^{B} is C₆₋₁₄ aryl or 5-14 membered heteroaryl. In certain embodiments, R^{A} is 5-14 membered heteroaryl and R^{B} is C₆₋₁₄ aryl or 5-14 membered heteroaryl. In certain embodiments, R^{A} is C₆₋₁₄ aryl or 5-14 membered heteroaryl and R^{B} is C₆₋₁₄ aryl. In certain embodiments, R^{A} is C₆₋₁₄ aryl or 5-14 membered heteroaryl and R^{B} is 5-14 membered heteroaryl.

In certain embodiments, both R^{A} and R^{B} are 06-14 aryl. In certain embodiments, both R^{A} and R^{B} are phenyl. In certain embodiments, R^{A} is C₆₋₁₄ aryl and R^{B} is C₃₋₁₀ carbocyclyl. In certain embodiments, R^{A} is C₆₋₁₄ aryl and R^{B} is 5-14 membered heteroaryl. In certain embodiments, R^{A} is C₆₋₁₄ aryl and R^{B} is 3-14 membered heterocyclyl. In certain embodiments, R^{A} is C₆₋₁₄ aryl and R^{B} and R^{C} together with the nitrogen (N) atom to which each is attached are joined to form a 5-14 membered carbocyclyl, heterocyclyl, aryl or heteroaryl ring. In certain embodiments, both R^{A} and R^{B} are 5-14 membered heteroaryl. In certain embodiments, R^{A} is 5-14 membered heteroaryl and R^{B} is C₃₋₁₀ carbocyclyl. In certain embodiments, R^{A} is 5-14 membered heteroaryl and R^{B} is C₆₋₁₄ aryl. In certain embodiments, R^{A} is 5-14 membered heteroaryl and R^{B} is 3-14 membered heterocyclyl. In certain embodiments, R^{A} is 5-14 membered heteroaryl and R^{B} and R^{C} together with the nitrogen (N) atom to which each is attached are joined to form a 5-14 membered carbocyclyl, heterocyclyl, aryl or heteroaryl ring. In certain embodiments, R^{A} is C₆₋₁₄ aryl; R^{B} and R^{C} together with the nitrogen (N) atom to which each is attached are joined to form a 5-14 membered carbocyclyl, heterocyclyl, aryl or heteroaryl ring; and X is selected from hydrogen, —CN, —CHO, —C(═O)R^{C1}, —C(═O)NR^{C2}₂, —CO₂H, —CO₂R^{C1}, —C(═NR^{C2})OR^{C1}, —C(═NR^{C2})NR^{C2}₂, —C(═S)NR^{C2}₂, —C(═O)SR^{C1}, —C(═S)SR^{C1}, C₁₋₁₀ perhaloalkyl, C₆₋₁₄ aryl, and 5-14 membered heteroaryl. In certain embodiments, R^{A} is C₆₋₁₄ aryl; R^{B} is C₆₋₁₄ aryl or 5-14 membered heteroaryl; R^{C} is an acyclic group; and X is selected from hydrogen, —CN, —CHO, —C(═O)R^{C1}, —C(═O)NR^{C2}₂, —CO₂H, —CO₂R^{C1}, —C(═NR^{C2})OR^{C1}, —C(═NR^{C2})NR^{C2}₂, —C(═S)NR^{C2}₂, —C(═O)SR^{C1}, —C(═S)SR^{C1}, C₁₋₁₀ perhaloalkyl, C₆₋₁₄ aryl, and 5-14 membered heteroaryl.

In certain embodiments, the compound is of the formula (XLIV):

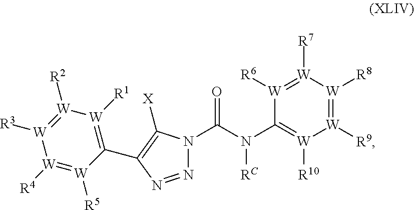

or a pharmaceutically acceptable form thereof; wherein X, R^{C}, W—R¹, W—R², W—R³, W—R⁴, W—R⁵, W—R⁶, W—R⁷, W—R⁸, W—R⁹, and W—R¹⁰ are as defined above and herein. In certain embodiments, at least one of R⁶, R⁷, R⁸, R⁹ and R¹⁰ of the formula (XLIV) is the group -L-R^{D} as defined above and herein. In certain embodiments, at least one of R⁶, R⁷, R⁸, R⁹ and R¹⁰ of the formula (XLIV) is further selected from the group —R^{E} as defined above and herein.

In certain embodiments, the compound is of the formulae (XLIV-A), (XLIV-B) or (XLIV-C):

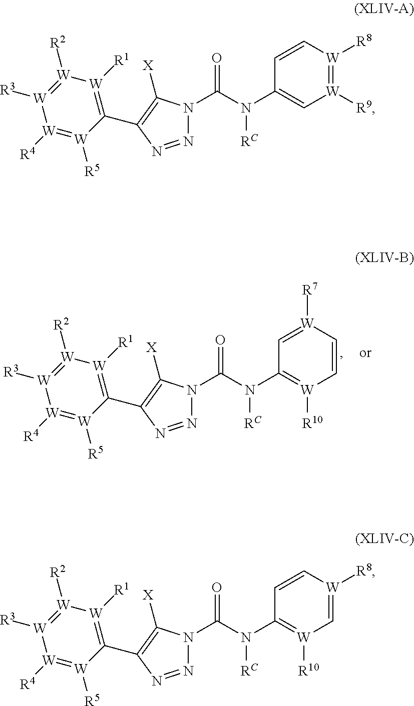

or a pharmaceutically acceptable form thereof; wherein X, R^{C}, W—R¹, W—R², W—R³, W—R⁴, W—R⁵, W—R⁷, W—R⁸, W—R⁹, and W—R¹⁰ are as defined above and herein. In certain embodiments, at least one of R⁷, R⁸, R⁹ and R¹⁰ of the formulae (II-a), (II-b) or (II-c) is the group -L-R^{D} as defined above and herein. In certain embodiments, at least one of R⁷, R⁸, R⁹ and R¹⁰ of the formulae (II-a), (II-b) or (II-c) is further selected from the group —R^{E} as defined above and herein.

In certain embodiments, the compound is of the formula (XLV):

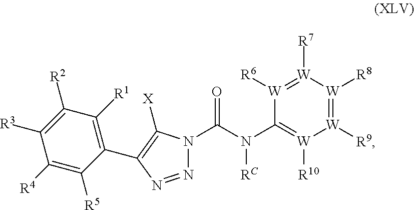

or a pharmaceutically acceptable form thereof; wherein X, R^{C}, R¹, R², R³, R⁴, R⁵, W—R⁶, W—R⁷, W—R⁸, W—R⁹, W—R¹⁰ defined are as defined above and herein. In certain embodiments, at least one of R⁶, R⁷, R⁸, R⁹ and R¹⁰ of the compound of formula (XLV) is the group -L-R^{D} as defined above and herein. In certain embodiments, at least one of R⁶, R⁷, R⁸, R⁹ and R¹⁰ of the compound of formula (XLV) is further selected from the group —R^{E} as defined above and herein.

In certain embodiments, the compound is of the formulae (XLV-A), (XLV-B), or (XLV-C):

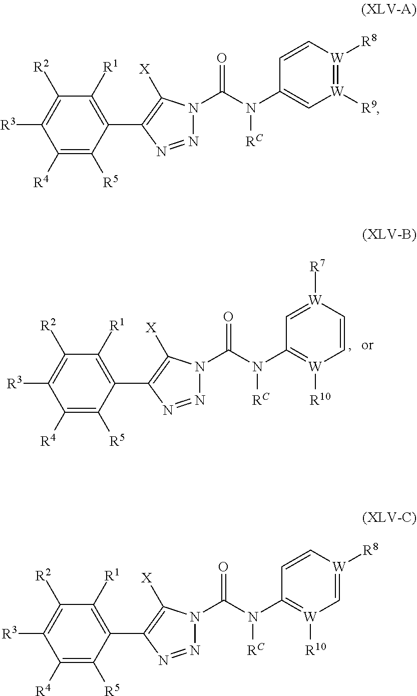

or a pharmaceutically acceptable form thereof; wherein X, R^{C}, R¹, R², R³, R⁴, R⁵, W—R⁷, W—R⁸, W—R⁹, and W—R¹⁰ are as defined above and herein. In certain embodiments, at least one of R⁷, R⁸, R⁹ and R¹⁰ of the formulae (III-a), (III-b) or (III-c) is the group -L-R^{D} as defined above and herein. In certain embodiments, at least one of R⁷, R⁸, R⁹ and R¹⁰ of formulae (III-a), (III-b) or (III-c) is further selected from the group —R^{E} as defined above and herein.

In certain embodiments, the compound is of the formula (XLVI):

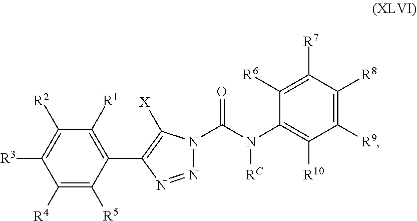

or a pharmaceutically acceptable form thereof; wherein X, R^{C}, R¹, R², R³, R⁴, R⁵, R⁶, R⁷, R⁸, R⁹, and R¹⁰ are as defined above and herein.

In certain embodiments, at least one of R⁶, R⁷, R⁸, R⁹ and R¹⁰ of the formula (XLVI) is the group -L-R^{D} as defined above and herein. In certain embodiments, at least one of R⁶, R⁷, R⁸, R⁹ and R¹⁰ of the formula (XLVI) is further selected from the group —R^{E} as defined above and herein. In certain embodiments, R¹-R⁵ are independently H, C₁₋₁₀ alkyl, C₁₋₁₀ alkyloxy, C₆₋₁₄ aryloxy, CN, —SO₂NR^{A7}₂, —SO₂R^{A6}, and —SO₂OR^{A6}; R^{C} is unsubstituted C₁₋₁₀ alkyl or unsubstituted C₃₋₁₀ carbocyclyl; and R⁶-R¹⁰ are independently selected from H, C₁₋₁₀ alkyl, C₁₋₁₀ alkyloxy, C₆₋₁₄ aryloxy, COOH, and —CO₂R^{A6}. In certain embodiments, R¹-R⁵ are independently H, methyl, methoxy, CN, and SO₂Me; R^{C} is unsubstituted C₁₋₃ alkyl or unsubstituted C₅₋₆ cycloalkyl; and R⁶-R¹⁰ are independently selected from H, methyl, methoxy, phenoxy, COOH, and CO₂Me.

In certain embodiments, the compound is of the formulae (XLVI-A), (XLVI-B), (XLVI-C), or (XLVI-A):

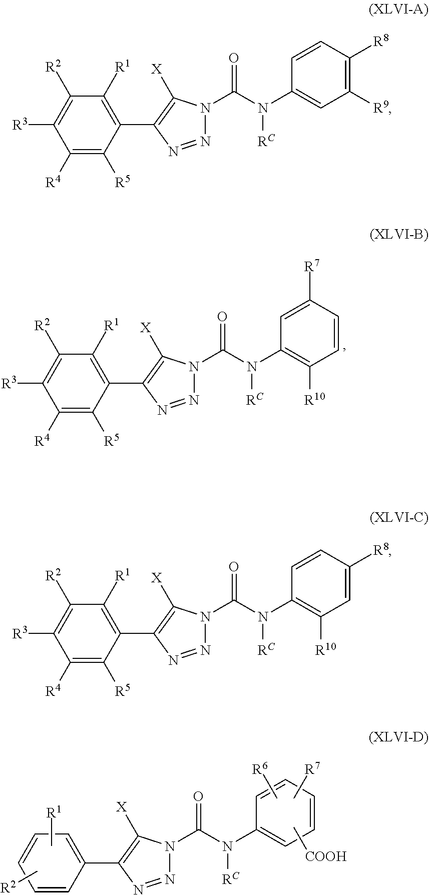

or a pharmaceutically acceptable form thereof; wherein X, R^{C}, R¹, R², R³, R⁴, R⁵, R⁶, R⁷, R⁸, R⁹, and R¹⁰ are as defined above and herein.

In certain embodiments, at least one of R⁷, R⁸, R⁹ and R¹⁰ of the formulae (XLVI-A), (XLVI-B) or (XLVI-C) is the group -L-R^{D} as defined above and herein. In certain embodiments, at least one of R⁷, R⁸, R⁹ and R¹⁰ of the formulae (XLVI-A), (XLVI-B), (XLVI-C) or (XLVI-D) is further selected from the group -R^{E} as defined above and herein. In one embodiment, provided herein is a compound of formula (XLVI-D), or a pharmaceutically acceptable form thereof. In one embodiment where the compound is of formula (XLVI-D), R^{C} is C₁₋₁₀ alkyl or C₃₋₁₀carbocyclyl. In one embodiment, R^{C} is ethyl, isopropyl, cyclopentyl or cyclohexyl. In another embodiment where the compound is of formula (XLVI-D), R¹ and R² are each independently hydrogen, halogen, —CN, —OR^{A1} or —SO₂R^{A1}, wherein R^{A1} is C₁₋₁₀ alkyl. In another embodiment, R¹ and R² are each independently hydrogen, fluoro, methoxy, —CN or —SO₂CH₃. In another embodiment where the compound is of formula (XLVI-D), R⁶ and R⁷ are each independently hydrogen, halogen or —O—R^{B1}, wherein R^{B1} is C₁₋₁₀ alkyl or C₆₋₁₄aryl. In another embodiment, R⁶ and R⁷ are each independently hydrogen, fluoro, methoxy or phenyloxy.

In certain embodiments, the compound is of the formula (XLVII):

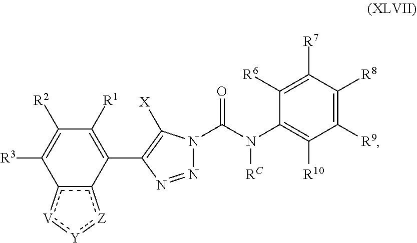

or a pharmaceutically acceptable form thereof; wherein X, R^{C}, V, Y, Z, R¹, R², R³, R⁶, R⁷, R⁸, R⁹, and R¹⁰ are as defined above and herein. In certain embodiments, at least one of R⁶, R⁷, R⁸, R⁹ and R¹⁰ of the formula (XLVII) is the group -L-R^{D} as defined above and herein. In certain embodiments, at least one of R⁶, R⁷, R⁸, R⁹ and R¹⁰ of the formula (XLVII) is further selected from the group —R^{E} as defined above and herein.

In certain embodiments, the compound is of the formulae (XLVII-A), (XLVII-B), or (XLVII-C):

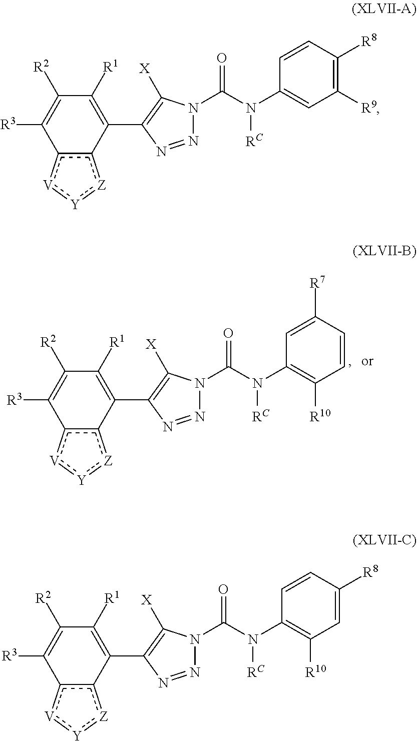

or a pharmaceutically acceptable form thereof; wherein X, R^{C}, V, Y, Z, R¹, R², R³, R⁷, R⁸, R⁹, and R¹⁰ are as defined above and herein. In certain embodiments, at least one of R⁷, R⁸, R⁹ and R¹⁰ of the formulae (V-a), (V-b) or (V-c) is the group -L-R^{D} as defined above and herein. In certain embodiments, at least one of R⁷, R⁸, R⁹ and R¹⁰ of the formulae (V-a), (V-b) or (V-c) is further selected from the group —R^{E} as defined above and herein.

In certain embodiments, the compound is of the formula (XLVIII):

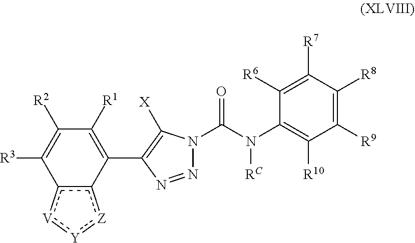

or a pharmaceutically acceptable form thereof; wherein R^{C}, Y, R¹, R², R³, R⁶, R⁷, R⁸, R⁹, and R¹⁰ are as defined above and herein. In certain embodiments, at least one of R⁶, R⁷, R⁸, R⁹ and R¹⁰ of the formula (XLVIII) is the group -L-R^{D} as defined above and herein. In certain embodiments, at least one of R⁶, R⁷, R⁸, R⁹ and R¹⁰ of the formula (XLVIII) is further selected from the group —R^{E} as defined above and herein. In certain embodiments, R¹-R³ are independently H, C₁₋₁₀ alkyl, C₁₋₁₀ alkyloxy, C₆₋₁₄ aryloxy, CN, —SO₂NR^{A7}₂, —SO₂R^{A6}, and —SO₂OR^{A6}; R^{C} is unsubstituted C₁₋₁₀ alkyl or unsubstituted C₃₋₁₀ carbocyclyl; and R⁶-R¹⁰ are independently selected from H, C₁₋₁₀ alkyl, C₁₋₁₀ alkyloxy, C₆₋₁₄ aryloxy, COOH, and —CO₂R^{A6}. In certain embodiments, R¹-R³ are independently H, methyl, methoxy, and CN; R^{C} is unsubstituted C₅₋₆ cycloalkyl; and R⁶-R¹⁰are independently selected from H, methyl, methoxy, phenoxy, COOH, and CO₂Me.

In certain embodiments, the compound is of the formulae (XLVIII-A), (XLVII-B), or (XLVIII-C):

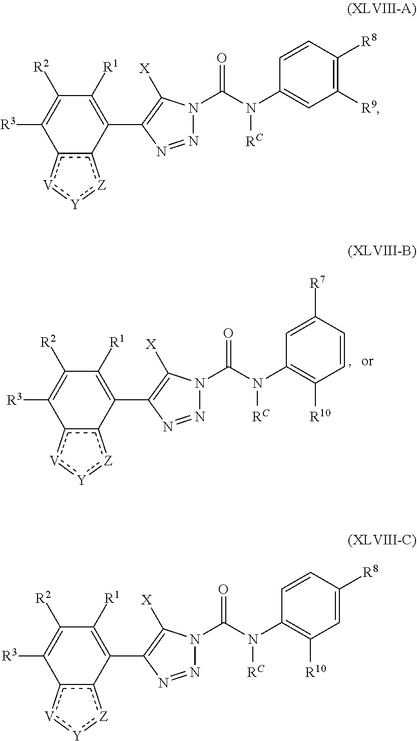

or a pharmaceutically acceptable form thereof; wherein R^{C}, Y, R¹, R², R³, R⁷, R⁸, R⁹, and R¹⁰ are as defined above and herein. In certain embodiments, at least one of R⁷, R⁸, R⁹ and R¹⁰ of the formulae (VI-a), (VI-b) or (VI-c) is the group -L-R^{D} as defined above and herein. In certain embodiments, at least one of R⁷, R⁸, R⁹ and R¹⁰ of the formulae (VI-a), (VI-b) or (VI-c) is further selected from the group —R^{E} as defined above and herein.

In some embodiments, the compound is one of the following:

| | Compound |
|---|---|
| 2038 | 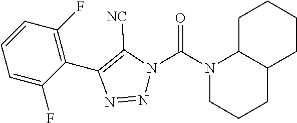 [SMILES: N#Cc1c(-c2c(F)cccc2F)nnn1C(=O)N1CCCC2CCCCC21] |
| 2039 | 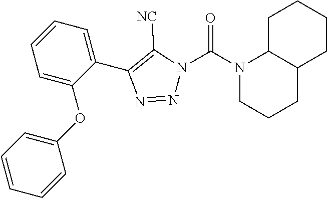 [SMILES: N#Cc1c(-c2ccccc2Oc2ccccc2)nnn1C(=O)N1CCCC2CCCCC21] |
| 2040 | 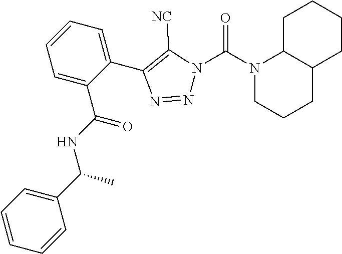 [SMILES: C[C@@H](NC(=O)c1ccccc1-c1nnn(C(=O)N2CCCC3CCCCC32)c1C#N)c1ccccc1] |
| 2041 | 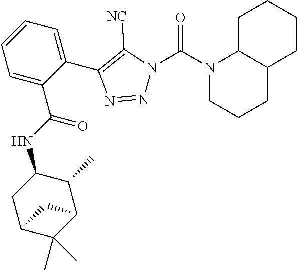 [SMILES: C[C@H]1[C@H](NC(=O)c2ccccc2-c2nnn(C(=O)N3CCCC4CCCCC43)c2C#N)C[C@H]2C[C@@H]1C2(C)C] |
| 2042 | 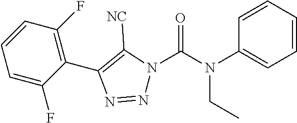 [SMILES: CCN(C(=O)n1nnc(-c2c(F)cccc2F)c1C#N)c1ccccc1] |
| 2043 | 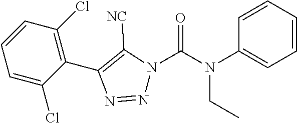 [SMILES: CCN(C(=O)n1nnc(-c2c(Cl)cccc2Cl)c1C#N)c1ccccc1] |
| 2044 | 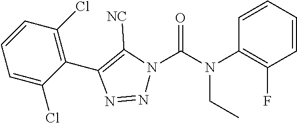 [SMILES: CCN(C(=O)n1nnc(-c2c(Cl)cccc2Cl)c1C#N)c1ccccc1F] |
| 2045 | 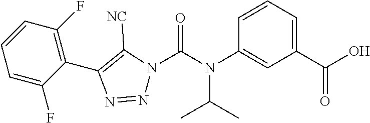 [SMILES: CC(C)N(C(=O)n1nnc(-c2c(F)cccc2F)c1C#N)c1cccc(C(=O)O)c1] |
| 2046 | 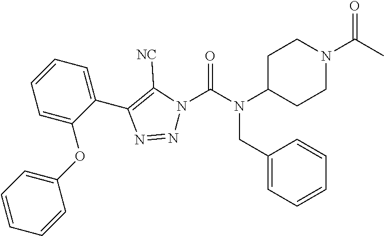 [SMILES: CC(=O)N1CCC(N(Cc2ccccc2)C(=O)n2nnc(-c3ccccc3Oc3ccccc3)c2C#N)CC1] |
| 2047 | 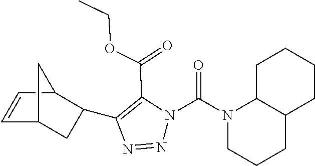 [SMILES: CCOC(=O)c1c(C2CC3C=CC2C3)nnn1C(=O)N1CCCC2CCCCC21] |
| 2048 | 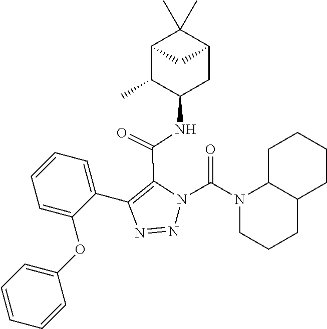 [SMILES: C[C@H]1[C@H](NC(=O)c2c(-c3ccccc3Oc3ccccc3)nnn2C(=O)N2CCCC3CCCCC32)C[C@H]2C[C@@H]1C2(C)C] |
| 2049 | 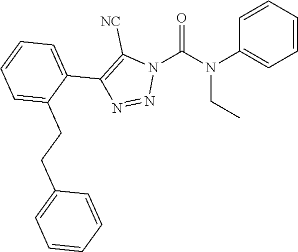 [SMILES: CCN(C(=O)n1nnc(-c2ccccc2CCc2ccccc2)c1C#N)c1ccccc1] |
| 2050 | 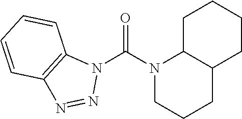 [SMILES: O=C(N1CCCC2CCCCC21)n1nnc2ccccc21] |
| 2051 | 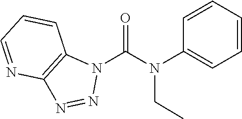 [SMILES: CCN(C(=O)n1nnc2ncccc21)c1ccccc1] |
| 2052 | 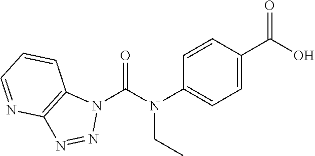 [SMILES: CCN(C(=O)n1nnc2ncccc21)c1ccc(C(=O)O)cc1] |
| 2053 | 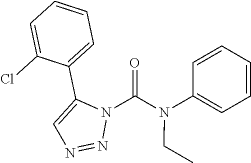 [SMILES: CCN(C(=O)n1nncc1-c1ccccc1Cl)c1ccccc1] |
| 2054 | 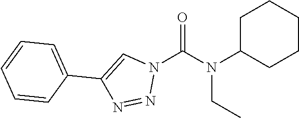 [SMILES: CCN(C(=O)n1cc(-c2ccccc2)nn1)C1CCCCC1] |
| 2055 | 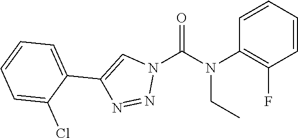 [SMILES: CCN(C(=O)n1cc(-c2ccccc2Cl)nn1)c1ccccc1F] |
| 2056 | 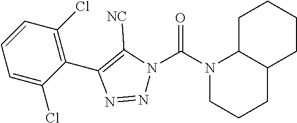 [SMILES: N#Cc1c(-c2c(Cl)cccc2Cl)nnn1C(=O)N1CCCC2CCCCC21] |
| 2057 | 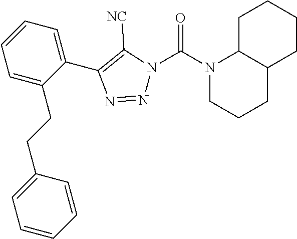 [SMILES: N#Cc1c(-c2ccccc2CCc2ccccc2)nnn1C(=O)N1CCCC2CCCCC21] |
| 2058 | 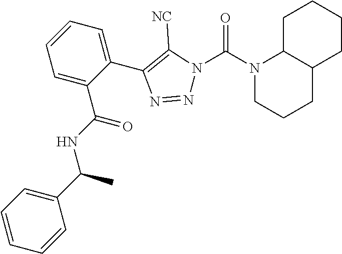 [SMILES: C[C@H](NC(=O)c1ccccc1-c1nnn(C(=O)N2CCCC3CCCCC32)c1C#N)c1ccccc1] |
| 2059 | 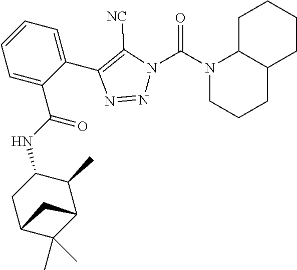 [SMILES: C[C@@H]1[C@@H](NC(=O)c2ccccc2-c2nnn(C(=O)N3CCCC4CCCCC43)c2C#N)C[C@@H]2C[C@H]1C2(C)C] |
| 2060 | 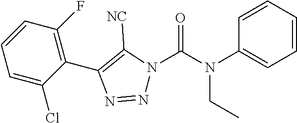 [SMILES: CCN(C(=O)n1nnc(-c2c(F)cccc2Cl)c1C#N)c1ccccc1] |
| 2061 | 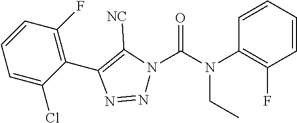 [SMILES: CCN(C(=O)n1nnc(-c2c(F)cccc2Cl)c1C#N)c1ccccc1F] |
| 2062 | 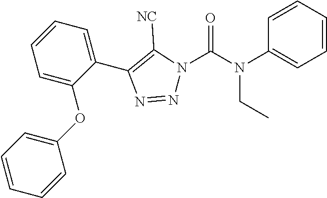 [SMILES: CCN(C(=O)n1nnc(-c2ccccc2Oc2ccccc2)c1C#N)c1ccccc1] |
| 2063 | 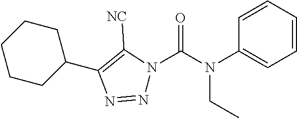 [SMILES: CCN(C(=O)n1nnc(C2CCCCC2)c1C#N)c1ccccc1] |
| 2064 | 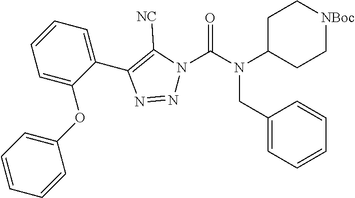 [SMILES: CC(C)(C)OC(=O)N1CCC(N(Cc2ccccc2)C(=O)n2nnc(-c3ccccc3Oc3ccccc3)c2C#N)CC1] |
| 2065 | 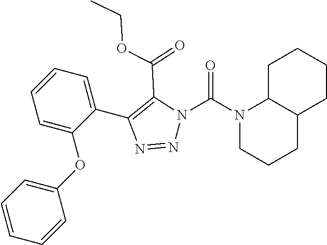 [SMILES: CCOC(=O)c1c(-c2ccccc2Oc2ccccc2)nnn1C(=O)N1CCCC2CCCCC21] |
| 2066 | 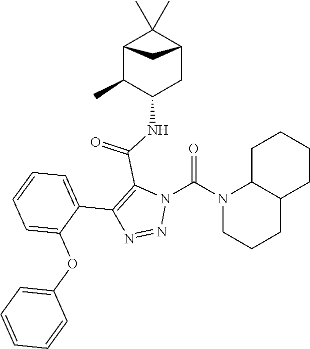 [SMILES: C[C@@H]1[C@@H](NC(=O)c2c(-c3ccccc3Oc3ccccc3)nnn2C(=O)N2CCCC3CCCCC32)C[C@@H]2C[C@H]1C2(C)C] |
| 2067 | 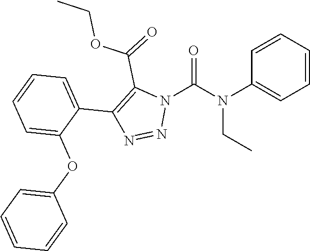 [SMILES: CCOC(=O)c1c(-c2ccccc2Oc2ccccc2)nnn1C(=O)N(CC)c1ccccc1] |
| 2068 | 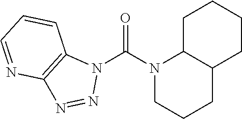 [SMILES: O=C(N1CCCC2CCCCC21)n1nnc2ncccc21] |
| 2069 | 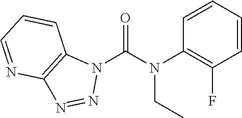 [SMILES: CCN(C(=O)n1nnc2ncccc21)c1ccccc1F] |
| 2070 | 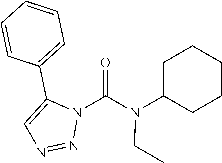 [SMILES: CCN(C(=O)n1nncc1-c1ccccc1)C1CCCCC1] |
| 2071 | 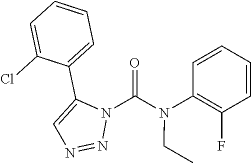 [SMILES: CCN(C(=O)n1nncc1-c1ccccc1Cl)c1ccccc1F] |
| 2072 | 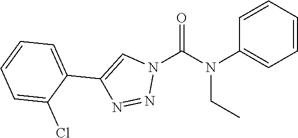 [SMILES: CCN(C(=O)n1cc(-c2ccccc2Cl)nn1)c1ccccc1] |
| 2073 | 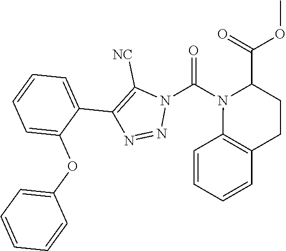 [SMILES: COC(=O)C1CCc2ccccc2N1C(=O)n1nnc(-c2ccccc2Oc2ccccc2)c1C#N] |

In some embodiments, the compound has the structure of Formula (XLIX):

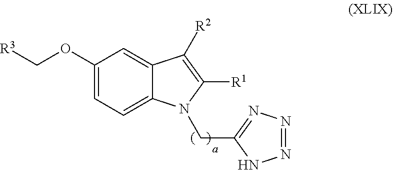

or a pharmaceutically acceptable salt thereof, wherein, R¹ is selected from the group consisting of H, CO₂R⁴, COR⁴, CONR⁵R⁶, CH(OH)R⁴, CR⁴═NOR⁴, heteroaryl and substituted heteroaryl; R² is selected from the group consisting of H, COR⁴, and CH(OH)R⁴; R³ is selected from the group consisting of aryl, substituted aryl, heteroaryl and substituted heteroaryl; R⁴ is H or lower alkyl; R⁵ and R⁶ are, independently, H, or lower alkyl or, together, form a 5 or 6 membered ring selected from the group consisting of piperidine, piperazine, pyrrolidine, morpholine and hydroxy piperidine; and n is an integer from 1 to 6.

In some embodiments, the compound is 5-(2,6-dichlorobenzyloxy)-1-[3-(1H-tetrazol-5-yl)propyl]-1H-indole-2-carboxylic acid ethyl ester; 1-{5-(2,6-dichlorobenzyloxy)-1-[3-(1H-tetrazol-5-yl)propyl]-1H-indol-2-yl}-1-morpholin-4-yl-methanone; 5-(2,6-dichlorobenzyloxy)-1-[3-(1H-tetrazol-5-yl)propyl]-1H-indole-2-carboxylic acid isobutyl amide; 5-(2,6-dichlorobenzyloxy)-1-[3-(1H-tetrazol-5-yl)propyl]-1H-indole-2-carboxylic acid diethylamide; 5-(2,6-dichlorobenzyloxy)-3-formyl-1-[3-(1H-tetrazol-5-yl)propyl]-1H-indole-2-carboxylic acid ethyl ester; 5-(2,6-dichlorobenzyloxy)-2-(3-methyl-[1,2,4]oxadiazol-5-yl)-1-[3-(1H-tetrazol-5-yl)propyl]-1H-indole; 5-(2,6-dichlorobenzyloxy)-1-[3-(1H-tetrazol-5-yl)propyl]-1H-indole; 1-{5-(2,6-dichlorobenzyloxy)-1-[3-(1H-tetrazol-5-yl)-propyl]-1H-indol-3-yl}propan-1-one; 5-(2,6-dichlorobenzyloxy)-1-[3-(1H-tetrazol-5-yl)propyl]-1H-indole-2-carbaldehyde-O-methyl oxime; or 5-(2,6-dichlorobenzyloxy)-2-(oxazol-5-yl)-1-[3-(1H-tetrazol-5-yl)propyl]-1H-indole; or a pharmaceutically acceptable salt thereof.

In some embodiments, the compound is one of the following:

| | Compound |
|---|---|
| 2074 | 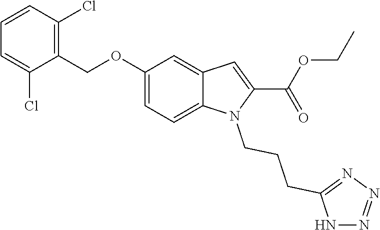 [SMILES: CCOC(=O)c1cc2cc(OCc3c(Cl)cccc3Cl)ccc2n1CCCc1nnn[nH]1] |
| 2075 | 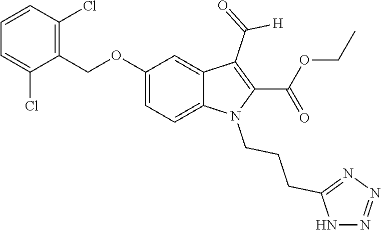 [SMILES: CCOC(=O)c1c(C=O)c2cc(OCc3c(Cl)cccc3Cl)ccc2n1CCCc1nnn[nH]1] |
| 2076 | 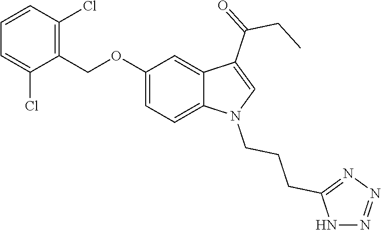 [SMILES: CCC(=O)c1cn(CCCc2nnn[nH]2)c2ccc(OCc3c(Cl)cccc3Cl)cc12] |
| 2077 | 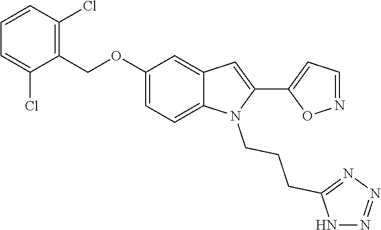 [SMILES: Clc1cccc(Cl)c1COc1ccc2c(c1)cc(-c1ccno1)n2CCCc1nnn[nH]1] |
| 2078 | 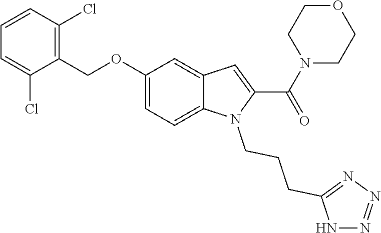 [SMILES: O=C(c1cc2cc(OCc3c(Cl)cccc3Cl)ccc2n1CCCc1nnn[nH]1)N1CCOCC1] |
| 2079 | 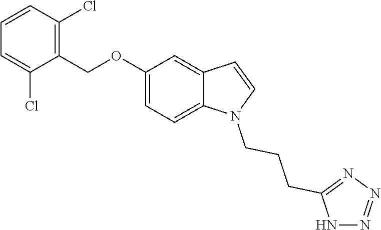 [SMILES: Clc1cccc(Cl)c1COc1ccc2c(ccn2CCCc2nnn[nH]2)c1] |
| 2080 | 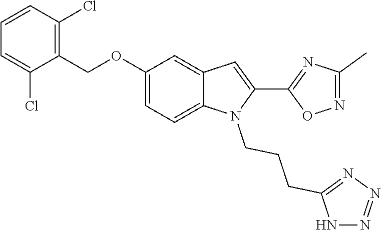 [SMILES: Cc1noc(-c2cc3cc(OCc4c(Cl)cccc4Cl)ccc3n2CCCc2nnn[nH]2)n1] |
| 2081 | 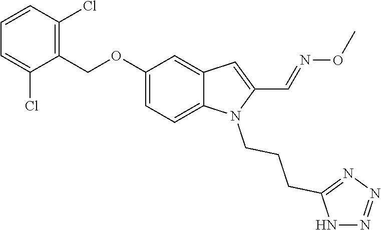 [SMILES: CO/N=C/c1cc2cc(OCc3c(Cl)cccc3Cl)ccc2n1CCCc1nnn[nH]1] |

In some embodiments, the compound has the structure of Formula (L):

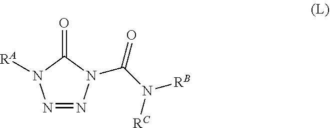

or a pharmaceutically acceptable form thereof; wherein: R^{A} is selected from C₃₋₁₀ carbocyclyl, 3-14 membered heterocyclyl, C₆₋₁₄ aryl and 5-14 membered heteroaryl; R^{B} is selected from C₁₋₁₀ alkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl and 5-14 membered heteroaryl; R^{C} is selected from hydrogen, —OH, —OR^{C1}, —ONR^{C2}₂, —NR^{C2}₂, —C(═O)R^{C1}, —CHO, —CO₂R^{C1}, —C(═O)NR^{C2}₂, —C(═NR^{C2})OR^{C1}, —C(═NR^{C2})NR^{C2}₂, —SO₂R^{C1}, —S(═O)R^{C1}, —Si(R^{C1})₃, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; wherein: each instance of R^{C1} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; and each instance of R^{C2} is, independently, selected from hydrogen, —OH, —OR, —NR^{C3}₂, —CN, —C(═O)R^{C1}, —C(═O)NR^{C3}₂, —CO₂R¹, —SO₂R^{C1}, —C(═NR^{C3})OR^{C1}, —C(═NR^{C3})NR^{C3}₂, —SO₂NR^{C3}₂, —SO₂R^{C3}, —SO₂OR^{C3}, —SOR^{C1}, —C(═S)NR^{C3}₂, —C(═O)SR^{C3}, —C(═S)SR^{C3}, —P(═O)₂R^{C1}, —P(═O)(R^{C1})₂, —P(═O)₂NR^{C3}₂, —P(═O)(NR^{C3})₂, C₁₋₁₀ alkyl, C₁₋₁₀ perhaloalkyl, C2-₁₀ alkenyl, C2-₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{C2} groups are joined to form a 3-14 membered heterocyclyl or 5-14 membered heteroaryl ring; or R^{B} and R^{C} together with the nitrogen (N) atom to which each is attached are joined to form a 5-14 membered heterocyclyl or heteroaryl ring.

In some embodiments, R^{A} is selected from C₆₋₁₄ aryl and 5-14 membered heteroaryl; R^{B} is selected from C₆₋₁₄ aryl and 5-14 membered heteroaryl; R^{c} is selected from —OH, —OR^{C1}, —ONR^{C2}₂, —NR^{C2}₂, —C(═O)R^{C1}, —CHO, —CO₂R^{C1}, —C(═O)NR^{C2}₂, —C(═NR^{C2})OR^{C1}, —C(═NR^{C2})NR^{C2}₂, —SO₂R^{C1}, —S(═O)R^{C1}, —Si(R^{C1})₃, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, with the proviso that R^{C} is not —CH₃; each instance of R^{C1} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each instance of R^{C2} is, independently, selected from hydrogen, —OH, —OR^{C1}, —NR^{C3}₂, —CN, —C(═O)R^{C1}, —C(═O)NR^{C3}₂, —CO₂R¹, —SO₂R¹, —C(═NR^{C3})OR^{C1}, —C(═NR^{C3})NR^{C3}₂, —SO₂NR^{C3}₂, —SO₂R^{C3}, —SO₂OR^{C3}, —SOR^{C1}, —C(═S)NR^{C3}₂, —C(═O)SR^{C3}, —C(═S)SR^{C3}, —P(═O)₂R^{C1}, —P(═O)(R^{C1})₂, —P(═O)₂NR^{C3}₂, —P(═O)(NR^{C3})₂, C₂₋₁₀ alkyl, C₂₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{C2} groups are joined to form a 3-14 membered heterocyclyl or 5-14 membered heteroaryl ring; or R^{B} and R^{C} together with the nitrogen (N) atom to which each is attached are joined to form a 5-14 membered ring; wherein: R^{B} is substituted with the group: -L-R^{D}; wherein: L is a covalent bond or a divalent C₁₋₁₀ hydrocarbon chain, wherein one, two or three methylene units of L are optionally and independently replaced with one or more —O—, —S—, —NR^{B8}—, —(C═NR^{B8})—, —C(═O)—, —C(═S)—, —S(═O)—, —S(═O)₂— divalent carbocyclyl, divalent heterocyclyl, divalent aryl or divalent heteroaryl group; R^{D} is selected from —CN, —NO₂, —N₃, —SO₂H, —SO₃H, —C(═O)R^{B7}, —CO₂H, —CHO, —C(OR^{B9})₂, —CO₂R^{B7}, —OC(═O)R^{B7}, —OCO₂R^{B7}, —C(═O)NR^{B8}₂, —OC(═O)NR^{B8}₂, NR^{B8}C(═O)R^{B7}, —NR^{B8}CO₂R^{B7}, —NR^{B8}C(═O)NR^{B8}₂, —C(═NR^{B8})OR^{B7}, —OC(═NR^{B8})R^{B7}, —OC(═NR^{B8})OR, —C(═NR^{B8})NR^{B8}₂, —OC(═NR^{B8})NR^{B8}₂, —NR^{B8}C(═NR^{B8})NR^{B8}₂, —C(═O)NR^{B8}SO₂R^{B7}, —NR^{B8}SO₂R^{B7}, —SO₂NR^{B8}₂, —SO₂R^{B7}, —SO₂OR^{B7}, —OSO₂R^{B7}, —S(═O)R^{B7}, —OS(═O)R^{B7}, —C(═S)NR^{B8}₂, —C(═O)SR^{B7}, —C(═S)SR^{B7}, —SC(═S)SR^{B7}, —P(═O)₂R^{B7}, —OP(═O)₂R^{B7}, —P(═O)(R^{B7})₂, —OP(═O)(R^{B7})₂, —OP(═O)(OR^{B9})₂, —P(═O)₂NR^{B8}₂, —OP(═O)₂NR^{B8}₂, —P(═O)(NR^{B8})₂, —OP(═O)(NR^{B8})₂, —NR^{B8}P(═O)(OR^{B9})₂, NR^{B8}P(═O)(NR^{B8})₂, —B(OR^{B9})₂, —BR^{B7}(OR^{B9}), and tetrazolyl; each instance of R^{B7} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each instance of R^{B8} is, independently, selected from hydrogen, —OH, —OR^{B7}, —NR^{B9}₂, —CN, —C(═O)R^{B7}, —C(═O)NR^{B9}₂, —CO₂R^{B7}, —SO₂R^{B7}, —C(═NR^{B9})OR^{B7}, —C(═NR^{B9})NR^{B9}₂, —SO₂NR^{B9}₂, —SO₂R^{B9}, —SO₂OR^{B9}, —SOR^{B7}, —C(═S)NR^{B9}₂, —C(═O)SR^{B9}, —C(═S)SR^{B9}, —P(═O)₂R^{B7}, —P(═O)(R^{B7})₂, —P(═O)₂NR^{B9}₂, —P(═O)(NR^{B9})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B8} groups are joined to form a 3-14 membered heterocyclyl or 5-14 membered heteroaryl ring; and each instance of R^{B9} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B9} groups are joined to form a 3-14 membered heterocyclyl or 5-14 membered heteroaryl ring.

In some embodiments, R^{A} is selected from C₃₋₁₀ carbocyclyl, 3-14 membered heterocyclyl, C₆₋₁₄ aryl and 5-14 membered heteroaryl; R^{B} is selected from C₁₋₁₀ alkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl and 5-14 membered heteroaryl; R^{C} is selected from hydrogen, —OH, —OR^{C1}, —ONR^{C2}₂, —NR^{C2}₂, —C(═O)R^{C1}, —CHO, —CO₂R^{C1}, —C(═O)NR^{C2}₂, —C(═NR^{C2})OR^{C1}, —C(═NR^{C2})NR^{C2}₂, —SO₂R^{C1}, —S(═O)R^{C1}, —Si(R^{C1})₃, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each instance of R^{C1} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; and each instance of R^{C2} is, independently, selected from hydrogen, —OH, —OR, —NR^{C3}₂, —CN, C(═O)R^{C1}, —C(═O)NR^{C3}₂, —CO₂R^{C1}, —SO₂R^{C1}, —C(═NR^{C3})OR¹, —C(═NR^{C3})NR^{C3}₂, —SO₂NR^{C3}₂, —SO₂R^{C3}, —SO₂OR^{C3}, —SOR^{C1}, —C(═S)NR^{C3}₂, —C(═O)SR^{C3}, —C(═S)SR^{C3}, —P(═O)₂R^{C1}, —P(═O)(R^{C1})₂, —P(═O)₂NR^{C3}₂, P(═O)(NR^{C3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{C2} groups are joined to form a 3-14 membered heterocyclyl or 5-14 membered heteroaryl ring; or R^{B} and R^{C} together with the nitrogen (N) atom to which each is attached are joined to form a 5-14 membered ring.

As described generally above, R^{A} is selected from C₃₋₁₀ carbocyclyl, 3-14 membered heterocyclyl, C₆₋₁₄ aryl, and 5-14 membered heteroaryl. In certain embodiments, R^{A} is C₃₋₁₀ carbocyclyl. Exemplary carbocyclyl groups include, but are not limited to, cyclopropyl (C₃), cyclobutyl (C₄), cyclopentyl (C₅), cyclopentenyl (C₅), cyclohexyl (C₆), cyclohexenyl (C₆), cyclohexadienyl (C₆), cycloheptyl (C₇), cycloheptadienyl (C₇), cycloheptatrienyl (C₇) and cyclooctyl (C₈). In certain embodiments, R^{A} is 3-14 membered heterocyclyl. Exemplary heterocyclyl groups include, but are not limited to, azirdinyl, oxiranyl, thiorenyl, azetidinyl, oxetanyl, thietanyl, tetrahydrofuranyl, dihydrofuranyl, tetrahydrothiophenyl, dihydrothiophenyl, pyrrolidinyl, dihydropyrrolyl, dioxolanyl, oxathiolanyl, dithiolanyl, piperidinyl, tetrahydropyranyl, dihydropyridinyl, thianyl, piperazinyl, morpholinyl, dithianyl, dioxanyl, azepanyl, oxepanyl thiepanyl, azocanyl, oxecanyl and thiocanyl. In certain embodiments, R^{A} is C₆₋₁₄ aryl. Exemplary aryl groups include, but are not limited to, phenyl, naphthyl and anthracyl. In certain embodiments, R^{A} is phenyl (C₆ aryl). In certain embodiments, R^{A} is naphthyl (C₁₀ aryl). In certain embodiments, R^{A} is 5-14 membered heteroaryl. In certain embodiments, R^{A} is 5-10 membered heteroaryl. In certain embodiments, R^{A} is 5-6 membered heteroaryl. In certain embodiments, R^{A} is 5,6-bicyclic heteroaryl. In certain embodiments, R^{A} is 6,6-bicyclic heteroaryl. In certain embodiments, R^{A} is a 5-membered heteroaryl group. Exemplary 5-membered heteroaryl groups include, but are not limited to, pyrrolyl, furanyl, thiophenyl, imidazolyl, pyrazolyl, oxazolyl, isoxazolyl, thiazolyl, isothiazolyl, triazolyl, oxadiazolyl, thiadiazolyl and tetrazolyl. In certain embodiments, R^{A} is a 6-membered heteroaryl group. Exemplary 6-membered heteroaryl groups include, but are not limited to, pyridinyl, pyridazinyl, pyrimidinyl, pyrazinyl, triazinyl and tetrazinyl. In certain embodiments, R^{A} is a 5,6-bicyclic heteroaryl group. Exemplary 5,6-bicyclic heteroaryl groups include, but are not limited to, indolyl, isoindolyl, indazolyl, benztriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. In certain embodiments, R^{A} is a 6,6-bicyclic heteroaryl group. Exemplary 6,6-bicyclic heteroaryl groups include, but are not limited to, naphthyridinyl, pteridinyl, quinolinyl, isoquinolinyl, cinnolinyl, quinoxalinyl, phthalazinyl and quinazolinyl.

In certain embodiments, R^{A} is a group of the formula (i):

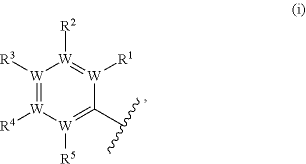

wherein each group W—R¹, W—R², W—R³, W—R⁴, and W—R⁵ independently represents either a nitrogen atom (N) or C—R¹, C—R², C—R³, C—R⁴, or C—R⁵, respectively; and wherein R¹, R², R³, R⁴ and R⁵ are independently selected from the group consisting of hydrogen, halogen, —CN, —NO₂, —N₃, —SO₂H, —SO₃H, —OH, —OR^{A1}, —ONR^{A2}₂, —NR^{A2}₂, —N(OR^{A3})R^{A3}, —SH, —SR^{A1}, —SSR^{A}, —C(═O)R^{A1}, —CO₂H, —CHO, —C(OR^{A3})₂, —CO₂R^{A1}, —OC(═O)R^{A1}, —OCO₂R^{A1}, —C(═O)NR^{A2}₂, —OC(═O)NR^{A2}₂, —NR^{A2}C(═O)R^{A1}, —NR^{A2}CO₂R^{A1}, —NR^{A2}C(═O)NR^{A2}₂, —C(═NR^{A2})OR^{A1}, —OC(═NR^{A2})R^{A1}, —OC(═NR^{A2})OR^{A1}, —C(═NR^{A2})NR^{A2}₂, —OC(═NR^{A2})NR^{A2}₂, —NR^{A2}C(═NR^{A2})NR^{A2}₂, —C(═O)NR^{A2}SO₂R^{A1}, —NR^{A2}SO₂R^{A1}, —SO₂N(^{A2})₂, —SO₂R^{A1}, —SO₂OR^{A1}, —OSO₂R^{A1}, —S(═O)R^{A1}, —OS(═O)R^{A1}, —Si(R^{A1})₃, —OSi(R^{A1})₃—C(═S)NR^{A2}₂, —C(═O)SR^{A1}, —C(═S)SR^{A1}, —SC(═S)SR^{A1}, —P(═O)₂R^{A1}, —OP(═O)₂R^{A1}, —P(═O)(R^{A1})₂, —OP(═O)(R^{A1})₂, —OP(═O)(OR^{A3})₂, —P(═O)₂NR^{A2}₂, —OP(═O)₂NR^{A2}₂, —P(═O)(NR^{A2})₂, —OP(═O)(NR^{A2})₂, —NR^{A2}P(═O)(OR^{A3})₂, —NR^{A2}P(═O)(NR^{A2})₂, —P(R^{A3})₂, —P(R^{A3})₃, —OP(^{A3})₂, —OP(^{A3})₃, —B(OR^{A3})₂, —BR^{A1}(OR^{A3}), C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; or one or more of R¹ and R², R² and R³, R³ and R⁴ or R⁴ and R⁵ are joined to form a C₃₋₁₀ carbocyclyl, 3-14 membered heterocyclyl, C₆₋₁₄ aryl or 5-14 membered heteroaryl ring; each instance of R^{A1} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each instance of R^{A2} is, independently, selected from hydrogen, —OH, —OR^{A1}, —NR^{A3}₂, —CN, —C(═O)R^{A1}, —C(═O)NR^{A3}₂, —CO₂R^{A1}, —SO₂R^{A1}, —C(═NR^{A3})OR^{A1}, —C(═NR^{A3})NR^{A3}₂, —SO₂NR^{A3}₂, —SO₂R^{A}, —SO₂OR^{A3}, —SOR^{A1}, —C(═S)NR^{A3}₂, —C(═O)SR^{A3}, —C(═S)SR^{A3}, —P(═O)₂R^{A1}, —P(═O)(R^{A1})₂, —P(═O)₂NR^{A3}₂, P(═O)NR^{A3}₂), C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{A2} groups are joined to form a 3-14 membered heterocyclyl or 5-14 membered heteroaryl ring; and each instance of R^{A3} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{A3} groups are joined to form a 3-14 membered heterocyclyl or 5-14 membered heteroaryl ring.

In certain embodiments, the group of formula (i) represents a C₆₋₁₄ aryl group or a 6-14 membered heteroaryl group. In certain embodiments, the group of formula (i) represents a 6-14 membered heteroaryl group. In certain embodiments, the group of formula (i) represents a C₆₋₁₄ aryl group. In certain embodiments, the C₆₋₁₄ aryl group of formula (i) represents a phenyl group.

As used herein, when one or more of R¹, R², R³, R⁴ and R⁵ is referred to as “not hydrogen”, it is meant that one or more of R¹, R², R³, R⁴ and R⁵ is independently selected from a group consisting of halogen, —CN, —NO₂, —N₃, —SO₂H, —SO₃H, —OH, —OR^{A1}, —ONR^{A2}₂, —NR^{A2}₂, —N(OR^{A3})R^{A3}, —SH, —SR^{A1}, —SSR^{A3}, —C(═O)R^{A1}, —CO₂H, —CHO, —C(OR^{A3})₂, —CO₂R^{A1}, —OC(═O)R^{A1}, —OCO₂R^{A1}, —C(═O)NR^{A2}₂, —OC(═O)NR^{A2}₂, —NR^{A2}C(═O)R^{A1}, —NR^{A2}CO₂R^{A1}, —NR^{A2}C(═O)NR^{A2}₂, —C(═NR^{A2})OR^{A1}, —OC(═NR^{A2})R^{A1}, —OC(═NR^{A2})OR^{A1}, —C(═NR^{A2})NR^{A2}₂, —OC(═NR^{A2})NR^{A2}₂, —NR^{A2}C(═NR^{A2})NR^{A2}₂, —C(═O)NR^{A2}SO₂R^{A1}, —NR^{A2}SO₂R^{A1}, —SO₂NR^{A2}₂, —SO₂R^{A1}, —SO₂OR^{A1}, —OSO₂R^{A1}, —S(═O)R^{A1}, —OS(═O)R^{A1}, —Si(R^{A1})₃, —OSi(R^{A1})₃—C(═S)NR^{A2}₂, —C(═O)SR^{A1}, —C(═S)SR^{A1}, —SC(S)SR^{A1}, —P(═O)₂R^{A1}, —OP(═O)₂R^{A1}, —P(═O)(R^{A1})₂, —OP(═O)(R^{A1})₂, —OP(═O)(OR^{A3})₂, —P(═O)₂NR^{A2}₂, —OP(═O)₂NR^{A2}₂, —P(═O)(NR^{A2})₂, —OP(═O)(NR^{A2})₂, —NR^{A2}P(═O)(OR^{A3})₂, —NR^{A2}P(═O)(NR^{A2})₂, —P(R^{A3})₂, —P(R^{A3})₃, —OP(^{A3})₂, —OP(R^{A3})₃, —B(OR^{A3})₂, or —BR^{A1}(OR^{A3}), C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; or one or more of R¹ and R², R² and R³, R³ and R⁴ or R⁴ and R⁵ are joined to form a C₃₋₁₀ carbocyclyl, 3-14 membered heterocyclyl, C₆₋₁₄ aryl or 5-14 membered heteroaryl ring.

In certain embodiments, R¹, R², R³, R⁴ and R⁵ are independently selected from the group consisting of hydrogen, halogen, —CN, —NO₂, —SO₂H, —SO₃H, —OH, —OR^{A1}, —NR^{A2}₂, —C(═O)R^{A1}, —CO₂H, —CHO, —C(OR^{A3})₂, —CO₂R^{A1}, —OC(═O)R^{A1}, —OCO₂R^{A1}, —C(═O)NR^{A2}₂, —OC(═O)NR^{A}₂, —NR^{A}C(═O)R^{A1}, —NR^{A}CO₂R^{A1}, —NR^{A}C(═O)NR^{A}₂, —C(═NR^{A})OR^{A1}, —OC(═NR^{A2})R^{A1}, —OC(═NR^{A2})OR^{A1}, —C(═NR^{A2})NR^{A2}₂, —OC(═NR^{A2})NR^{A2}₂, —NR^{A2}C(═NR^{A2})NR^{A2}₂, —C(═O)NR^{A2}SO₂R^{A1}, —NR^{A2}SO₂R^{A1}, —SO₂NR^{A2}₂, —SO₂R^{A1}, —SO₂OR^{A1}, —OSO₂R^{A1}, —S(═O)R^{A1}, —OS(═O)R^{A1}, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; or one or more of R¹ and R², R² and R³, R³ and R⁴, or R⁴ and R⁵ are joined to form a C₃₋₁₀ carbocyclyl, 3-14 membered heterocyclyl, C₆₋₁₄ aryl or 5-14 membered heteroaryl ring.

In certain embodiments, R¹, R², R³, R⁴ and R⁵ are independently selected from the group consisting of hydrogen, halogen, —CN, —OR^{A1}, —NR^{A2}₂, —CO₂H, —CO₂R^{A1}, —C(═O)NR^{A2}₂, —SO₂R^{A1}, C₁₋₁₀ alkyl, C₂₋₁₀ alkynyl, 3-14 membered heterocyclyl, and C₆₋₁₄ aryl; or one or more of R¹ and R², R² and R³, R³ and R⁴ or R⁴ and R⁵ are joined to form a 5-14 membered heteroaryl ring. In certain embodiments, R¹, R², R³, R⁴ and R⁵ are independently selected from the group consisting of hydrogen, halogen, —OR^{A1}, —NR^{A2}₂, —CO₂H, —C(═O)NR^{A2}₂, —SO₂R^{A1}, and 3-14 membered heterocyclyl; or R⁴ and R⁵ are joined to form a 5-14 membered heteroaryl ring. In certain embodiments, R¹, R², R³, R⁴ and R⁵ are independently selected from the group consisting of hydrogen, halogen, —OR^{A1}, and —C(═O)NR^{A2}₂; or R⁴ and R⁵ are joined to form a 5-14 membered heteroaryl ring. In certain embodiments, R¹, R², R³, R⁴ and R⁵ are independently selected from the group consisting of hydrogen, halogen, —OR^{A1}, and —C(═O)NR^{A2}₂; or R⁴ and R⁵ are joined to form a 5-14 membered heteroaryl ring. In certain embodiments, R¹, R², R³, R⁴ and R⁵ are independently selected from the group consisting of hydrogen, halogen, and —OR^{A1}. In certain embodiments, R¹, R², R³, R⁴ and R⁵ are independently selected from the group consisting of hydrogen, fluoro, chloro, and —OR^{A1}. In certain embodiments, R¹, R², R³, R⁴ and R⁵ are independently selected from the group consisting of hydrogen, fluoro, chloro, and —OMe. In certain embodiments, R¹, R², R³, R⁴ and R⁵ are independently selected from the group consisting of hydrogen, fluoro and —OR^{A1}. In certain embodiments, R¹, R², R³, R⁴ and R⁵ are independently selected from the group consisting of hydrogen, fluoro and —OMe. In certain embodiments, R¹, R², R³, R⁴ and R⁵ are independently selected from the group consisting of hydrogen and fluoro. In certain embodiments, R¹, R², R³, R⁴ and R⁵ are independently selected from the group consisting of hydrogen and chloro. In certain embodiments, R⁴ and R⁵ are joined to form a 5-14 membered heteroaryl ring.

In certain embodiment R^{A} is a group of the formula (ii):

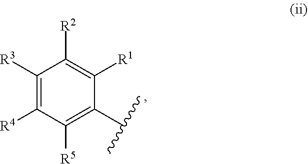

wherein R¹, R², R³, R⁴ and R⁵ are as defined above and herein.

In certain embodiments, the group of formula (ii) represents a C₆₋₁₄ aryl group. In certain embodiments, the C₆₋₁₄ aryl group of formula (ii) represents a phenyl group. In certain embodiments, R^{A} is a monosubstituted, disubstituted or trisubstituted group of the formula (ii). In certain embodiments, R^{A} is a monosubstituted or disubstituted group of the formula (ii). In certain embodiments, R^{A} is a monosubstituted group of the formula (ii). For example, in certain embodiments, R^{A} is an ortho-substituted group of the formula (ii), e.g., wherein R—R⁴ are hydrogen, and R⁵ is not hydrogen. In certain embodiments, R^{A} is a meta-substituted group of the formula (ii), e.g., wherein R^{x}-R³ and R⁵ are hydrogen and R⁴ is not hydrogen. In certain embodiments, R^{A} is a para-substituted group of the formula (ii), e.g., wherein R¹, R², R⁴ and R⁵ are hydrogen and R³ is not hydrogen.

In certain embodiments, R^{A} is a disubstituted group of the formula (ii). For example, in certain embodiments, R^{A} is a 2,6-disubstituted group of the formula (ii), e.g., wherein R², R³ and R⁴ are hydrogen, and R¹ and R⁵ are not hydrogen. In certain embodiments, R^{A} is a 2,5-disubstituted group of the formula (ii), e.g., wherein R², R³ and R⁵ are hydrogen, and R¹ and R⁴ are not hydrogen. In certain embodiments, R^{A} is a 2,4-disubstituted group of the formula (ii), e.g., wherein R², R³ and R⁵ are hydrogen, and R¹ and R³ are not hydrogen. In certain embodiments, R^{A} is a 2,3-disubstituted group of the formula (ii), e.g., wherein R¹, R² and R³ are hydrogen, and R⁴ and R⁵ are not hydrogen. In certain embodiments, R^{A} is a 3,4-disubstituted group of the formula (ii), e.g., wherein R¹, R⁴ and R⁵ are hydrogen, and R² and R³ are not hydrogen. In certain embodiments, R^{A} is a 3,5-disubstituted group of the formula (ii), e.g., wherein R¹, R³ and R⁵ are hydrogen, and R² and R⁴ are not hydrogen. For example, in certain embodiments, R^{A} is a 2,6-disubstituted group as described herein. In certain embodiments, one of R¹ and R⁵ is halogen, —CN, —OR^{A1}, —NR^{A2}₂,—CO₂H, —CO₂R^{A1}, —C(═O)NR^{A2}₂, —SO₂R^{A1}, C₁₋₁₀ alkyl, C₂₋₁₀ alkynyl, 3-14 membered heterocyclyl, and C₆₋₁₄ aryl, and the other of R¹ and R⁵ is halogen, —CN, —OR^{A1}, —NR^{A2}₂, —C₂H, —CO₂R^{A1}, —C(═O)NR^{A2}₂, —SO₂R^{A1}, C₁₋₁₀ alkyl, C₂₋₁₀ alkynyl, 3-14 membered heterocyclyl, and C₆₋₁₄ aryl.

In certain embodiments, one of R¹ and R⁵ is halogen, —OR^{A1}, C₁₋₁₀ alkyl, or —C(═O)NR^{A2}₂, and the other of R¹ and R⁵ is halogen, —OR^{A1}, C₁₋₁₀ alkyl, or —C(═O)NR^{A2}₂. In certain embodiments, each of R¹ and R⁵ is independently halogen. For example, each of R¹ and R⁵ is independently selected from fluoro and chloro.

In certain embodiments, R^{A} is a trisubstituted group of the formula (ii). For example, in certain embodiments, R^{A} is a 2,4,6-trisubstituted group of the formula (ii), e.g., wherein R² and R⁴ are hydrogen, and R¹, R³ and R⁵ are not hydrogen. In certain embodiments, R^{A} is a 2,3,6-trisubstituted group of the formula (ii), e.g., wherein R² and R³ are hydrogen, and R¹, R⁴ and R⁵ are not hydrogen. In certain embodiments, R^{A} is a 2,4,5-trisubstituted group of the formula (ii), e.g., wherein R² and R⁵ are hydrogen, and R¹, R³ and R⁴ are not hydrogen. In certain embodiments, R^{A} is a 2,3,4-trisubstituted group of the formula (ii), e.g., wherein R⁴ and R⁵ are hydrogen, and R¹, R² and R³ are not hydrogen. In certain embodiments, R^{A} is a 3,4,5-trisubstituted group of the formula (ii), e.g., wherein R¹ and R⁵ are hydrogen, and R², R³ and R⁴ are not hydrogen.

In certain embodiments, R^{A} is heteroaryl selected from a 5-6-membered heteroaryl, a 5,6-bicyclic heteroaryl or a 6,6-bicyclic heteroaryl. In certain embodiments, R^{A} is a 6-membered heteroaryl. In certain embodiments, R^{A} is a 6-membered heteroaryl selected from pyridinyl. In certain embodiments, R^{A} is 2-pyridinyl, 3-pyridinyl or 4-pyridinyl.

In certain embodiments, R^{A} is a 2-pyridinyl wherein W—R¹ is N, and W—R², W—R³, W—R⁴, and W—R⁵ are C—R², C—R³, C—R⁴ and C—R⁵, respectively, e.g., of the formula

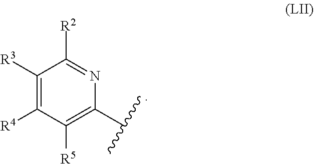

In certain embodiments, R^{A} is a 3-pyridinyl wherein W—R² is N, and W—R¹, W—R³, W—R⁴, and W—R⁵ are C—R¹, C—R³, C—R⁴ and C—R⁵, respectively, e.g., of the formula

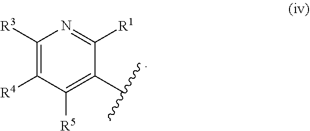

In certain embodiments, R^{A} is a 4-pyridinyl wherein W—R³ is N, and W—R¹, W—R², W—R⁴, and W—R⁵ are C—R¹, C—R², C—R⁴ and C—R⁵, respectively, e.g., of the formula

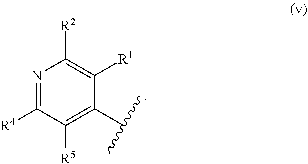

In certain embodiments, R^{A} is a monosubstituted or disubstituted pyridinyl. In certain embodiments, R^{A} is a monosubstituted pyridinyl. In certain embodiments, R^{A} is a monosubstituted pyridinyl of the formula (LII) wherein R³, R⁴, R⁵ are hydrogen and R² is not hydrogen. In certain embodiments, R^{A} is a monosubstituted pyridinyl of the formula (LII) wherein R², R⁴, R⁵ are hydrogen and R³ is not hydrogen. In certain embodiments, R^{A} is a monosubstituted pyridinyl of the formula (LII) wherein R², R³, R⁵ are hydrogen and R⁴ is not hydrogen. In certain embodiments, R^{A} is a monosubstituted pyridinyl of the formula (LII) wherein R², R³, R⁴ are hydrogen and R⁵ is not hydrogen. In certain embodiments, R^{A} is a monosubstituted pyridinyl of the formula (iv) wherein R³, R⁴, R⁵ are hydrogen and R¹ is not hydrogen. In certain embodiments, R^{A} is a monosubstituted pyridinyl of the formula (iv) wherein R¹, R⁴, R⁵ are hydrogen and R³ is not hydrogen. In certain embodiments, R^{A} is a monosubstituted pyridinyl of the formula (iv) wherein R¹, R³, R⁵ are hydrogen and R⁴ is not hydrogen. In certain embodiments, R^{A} is a monosubstituted pyridinyl of the formula (iv) wherein, R³, R⁴ are hydrogen and R⁵ is not hydrogen. In certain embodiments, R^{A} is a monosubstituted pyridinyl of the formula (v) wherein R², R⁴, R⁵ are hydrogen and R¹ is not hydrogen. In certain embodiments, R^{A} is a monosubstituted pyridinyl of the formula (v) wherein R¹, R⁴, R⁵ are hydrogen and R² is not hydrogen.

In certain embodiments, R^{A} is a disubstituted pyridinyl. In certain embodiments, R^{A} is a disubstituted pyridinyl of the formula (LII) wherein R³ and R⁴ are hydrogen and R² and R⁵ are not hydrogen. In certain embodiments, R^{A} is a disubstituted pyridinyl of the formula (LII) wherein R² and R⁴ are hydrogen and R³ and R⁵ are not hydrogen. In certain embodiments, R^{A} is a disubstituted pyridinyl of the formula (LII) wherein R² and R³ are hydrogen and R⁴ and R⁵ are not hydrogen. In certain embodiments, R^{A} is a disubstituted pyridinyl of the formula (LII) wherein R³ and R⁵ are hydrogen and R² and R⁴ are not hydrogen. In certain embodiments, R^{A} is a disubstituted pyridinyl of the formula (LII) wherein R⁴ and R⁵ are hydrogen and R² and R³ are not hydrogen. In certain embodiments, R^{A} is a disubstituted pyridinyl of the formula (LII) wherein R² and R⁵ are hydrogen and R³ and R⁴ are not hydrogen. In certain embodiments, R^{A} is a disubstituted pyridinyl of the formula (iv) wherein R³ and R⁴ are hydrogen and R¹ and R⁵ are not hydrogen. In certain embodiments, R^{A} is a disubstituted pyridinyl of the formula (iv) wherein R³ and R⁵ are hydrogen and R¹ and R⁴ are not hydrogen. In certain embodiments, R^{A} is a disubstituted pyridinyl of the formula (iv) wherein R⁴ and R⁵ are hydrogen and R¹ and R³ are not hydrogen. In certain embodiments, R^{A} is a disubstituted pyridinyl of the formula (iv) wherein R¹ and R⁴ are hydrogen and R³ and R⁵ are not hydrogen. In certain embodiments, R^{A} is a disubstituted pyridinyl of the formula (iv) wherein R¹ and R⁵ are hydrogen and R³ and R⁴ are not hydrogen. In certain embodiments, R^{A} is a disubstituted pyridinyl of the formula (iv) wherein R¹ and R³ are hydrogen and R⁴ and R⁵ are not hydrogen. In certain embodiments, R^{A} is a disubstituted pyridinyl of the formula (v) wherein R² and R⁴ are hydrogen and R¹ and R⁵ are not hydrogen. In certain embodiments, R^{A} is a disubstituted pyridinyl of the formula (v) wherein R⁴ and R⁵ are hydrogen and R¹ and R² are not hydrogen. In certain embodiments, R^{A} is a disubstituted pyridinyl of the formula (v) wherein R² and R⁵ are hydrogen and R¹ and R⁴ are not hydrogen. In certain embodiments, R^{A} is a disubstituted pyridinyl of the formula (v) wherein R¹ and R⁵ are hydrogen and R² and R⁴ are not hydrogen.

In certain embodiments, R^{A} is a 5,6-bicyclic heteroaryl. For example, in certain embodiments, R^{A} is a 5,6-bicyclic heteroaryl group of the formula

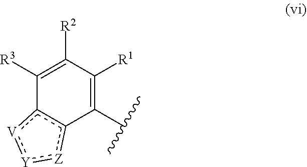

wherein R¹, R², R³ are as defined above and herein and R⁴ and R⁵ are joined to form a 5-membered heteroaryl ring; X, Y and Z are independently selected from CR^{A4}, O, S, N, or NR^{A5}; each instance of R^{A4} is, independently, selected from hydrogen, halogen, —CN, —NO₂, —N₃, —SO₂H, —SO₃H, —OH, —OR^{A6}, —ONR^{A7}₂, —NR^{A7}₂, —N(OR^{A6})R^{A8}, —SH, —SR^{A6}, —SSR^{A8}, —C(═O)R^{A6}, —CO₂H, —CHO, —C(OR^{A8})₂, —CO₂R^{A6}, —OC(═O)R^{A6}, —OCO₂R^{A6}, —C(═O)NR^{A7}₂, —OC(═O)NR^{A7}₂, —NR^{A7}C(═O)R^{A6}, —NR^{A7}CO₂R^{A6}, —NR^{A7}C(═O)NR^{A7}₂, —C(═NR^{A7})OR^{A6}, —OC(═NR^{A7})R^{A6}, —OC(═NR^{A7})OR^{A6}, —C(═NR^{A7})NR^{A7}₂, —OC(═NR^{A7})NR^{A7}₂, —NR^{A7}C(═NR^{A7})NR^{A7}₂, —C(═O)NR^{A7}SO₂R^{A6}, —NR^{A7}SO₂R^{A6}, —SO₂NR^{A7}₂, —SO₂R^{A6},—SO₂OR^{A6}, —OSO2R, —S(═O)R^{A6}, —OS(═O)R^{A6}, —Si(R^{A6})₃, —OSi(R^{A6})₃, —C(═S)NR^{A7}₂, —C(═O)SR^{A6}, —C(═S)SR^{A6}, —SC(═S)SR^{A6}, —P(═O)₂R^{A6}, —OP(═O)₂R^{A6}, —P(═O)(R^{A6})₂, —OP(═O)(R^{A6})₂, —OP(═O)(OR^{A8})₂, —P(═O)₂NR^{A7}₂, —OP(═O)₂NR^{A7}₂, —P(═O)(NR^{A7})₂, —OP(═O)(NR^{A7})₂, —NR^{A7}P(═O)(OR^{A8})₂, —NR^{A7}P(═O)(NR^{A7})₂, —P(R^{A8})₂, —P(R^{A8})₃, —OP(R^{A8})₂, —OP(R^{A8})₃, —B(OR^{A8})₂, or —BR^{A6}(OR^{A8}), C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each instance of R^{A6} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each instance of R^{A5} and R^{A7} is, independently, selected from hydrogen, —OH, —OR^{A6}, —NR^{A7}₂, —CN, —C(═O)R^{A6}, —C(═O)NR^{A7}₂, —CO₂R^{A6}, —SO₂R^{A7}, —C(═NR^{A3})OR^{A6}, —C(═NR^{A7})NR^{A7}₂, —SO₂NR^{A3}₂, —SO₂R^{A6}, —SO₂OR^{A8}, —SOR^{A6}, —C(═S)NR^{A7}₂, —C(═O)SR^{A8}, —C(═S)SR^{A8}, —P(═O)₂R^{A6}, —P(═O)(R^{A6})₂, —P(═O)₂NR^{A8}₂, —P(═O)(NR^{A8})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{A7} groups are joined to form a 3-14 membered heterocyclyl or 5-14 membered heteroaryl ring; each instance of R^{A8} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R groups are joined to form a 3-14 membered heterocyclyl or 5-14 membered heteroaryl ring; and the dashed line represents a double or single bond.

In certain embodiments, R¹ is hydrogen. In certain embodiments, R² is hydrogen. In certain embodiments, R³ is hydrogen. In certain embodiments, R¹, R² and R³ are hydrogen.

In certain embodiments, R^{A} is a heteroaryl group of

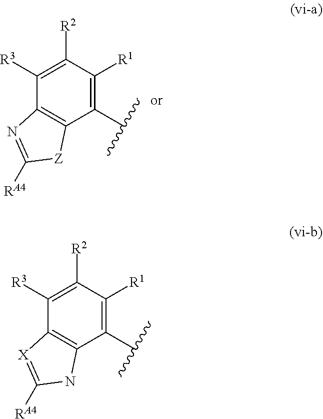

wherein R¹, R², R³ are as defined above and X and Z are independently selected from O, S and NR^{A5}. In certain embodiments, wherein R^{A} is a heteroaryl group of the formulae (vi-a) or (vi-b), X and Z are O (i.e., benzoxazolyl). In certain embodiments, X and Z are S (i.e., benzthiazolyl). In certain embodiments, X and Z are NR^{A5} (i.e., imidazolyl).

In certain embodiments, R^{A} is a heteroaryl group of

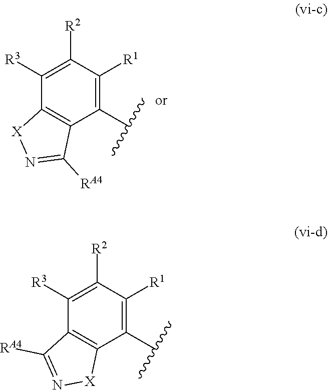

wherein R¹, R², R³ are as defined above and X is independently selected from O, S and NR^{A5}.

In certain embodiments, wherein R^{A} is a heteroaryl group of the formulae (vi-c) or (vi-d), X is O (i.e., benzisoxazolyl). In certain embodiments, X is S (i.e., benzisothiazolyl). In certain embodiments, X is NR^{A5} (i.e., indazolyl).

In certain embodiments R^{A} is a heteroaryl group of the

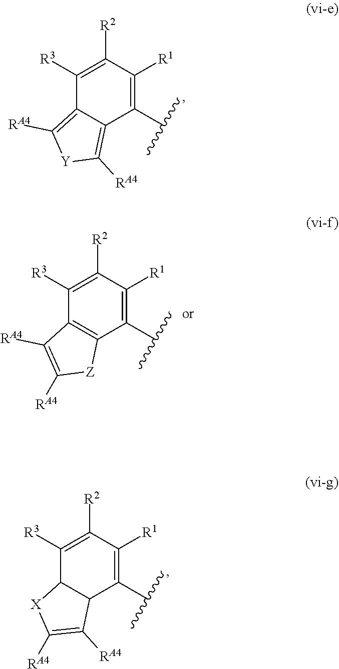

wherein R¹, R², R³ and R^{A4} are as defined above and X, Y and Z are independently selected from O, S and NR^{A5}.

In certain embodiments, wherein R^{A} is a heteroaryl group of the formulae (vi-e), (vi-f) or (vi-g), Y is O (i.e., benzofuranyl or isobenzofuranyl). In certain embodiments, Y is S (i.e., benzothiophenyl or isobenzothiophenyl). In certain embodiments, Y is NR^{A5} (i.e., indolyl or isoindolyl).

In certain embodiment R^{A} is a heteroaryl group of

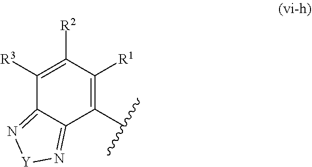

wherein R¹, R², R³ are as defined above and Y is independently selected from O, S and NR^{A5}.

In certain embodiments, wherein R^{A} is a heteroaryl group of the formula (vi-e), Y is O (i.e., benzoxadiazolyl). In certain embodiments, Y is S (i.e., benzthiadiazolyl). In certain embodiments, Y is NR^{A5} (i.e., benztriazolyl).

As described generally above, R^{B} is selected from C₁₋₁₀ alkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl and 5-14 membered heteroaryl; or R and R together with the nitrogen (N) atom to which each is attached are joined to form a 5-14 membered ring. In certain embodiments, R^{B} is selected from C₁₋₁₀ alkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl and 5-14 membered heteroaryl. In certain embodiments, R^{B} is an acyclic group, i.e., selected from C₁₋₁₀ alkyl, C₂₋₁₀ alkenyl, C₂₋₁₀ alkynyl and 3-14 membered heteroaliphatic. In certain embodiments, R^{B} is C₁₋₁₀ alkyl. In certain embodiments, R^{B} is a substituted C₁₋₁₀ alkyl, e.g., a C₁₋₁₀ aralkyl group. In certain embodiments, R^{B} is a C₁₋₂ aralkyl, e.g., for example, a substituted or unsubstituted benzyl group (C₁ aralkyl) or substituted or unsubstituted phenylethyl group (C₂ aralkyl). In certain embodiments, R^{B} is a C₁₋₁₀ heteroaralkyl. In certain embodiments, R^{B} is alkenyl. In certain embodiments, R^{B} is alkynyl. In certain embodiments, R^{B} is 3-14 membered heteroaliphatic. Alternatively, in certain embodiments, R^{B} is a cyclic group, i.e., selected from C₃₋₁₀ carbocyclyl, 3-14 membered heterocyclyl, C₆₋₁₄ aryl and 5-14 membered heteroaryl. In certain embodiments, R^{B} is C₃₋₁₀ carbocyclyl or 3-14 membered heterocyclyl. In certain embodiments, R^{B} is C₃₋₁₀ carbocyclyl. Exemplary carbocyclyl groups include, but are not limited to, cyclopropyl (C₃), cyclobutyl (C₄), cyclopentyl (C₅), cyclopentenyl (C₅), cyclohexyl (C₆), cyclohexenyl (C₆), cyclohexadienyl (C₆), cycloheptyl (C₇), cycloheptadienyl (C₇), cycloheptatrienyl (C₇) and cyclooctyl (C₈). In certain embodiments, R^{B} is 3-14 membered heterocyclyl. Exemplary heterocyclyl groups include, but are not limited to, azirdinyl, oxiranyl, thiorenyl, azetidinyl, oxetanyl, thietanyl, tetrahydrofuranyl, dihydrofuranyl, tetrahydrothiophenyl, dihydrothiophenyl, pyrrolidinyl, dihydropyrrolyl, dioxolanyl, oxathiolanyl, dithiolanyl, piperidinyl, tetrahydropyranyl, dihydropyridinyl, thianyl, piperazinyl, morpholinyl, dithianyl, dioxanyl, azepanyl, oxepanyl thiepanyl, azocanyl, oxecanyl and thiocanyl. In certain embodiments, R^{B} is C₆₋₁₄ aryl or 5-14 membered heteroaryl. In certain embodiments, R^{B} is C₆₋₁₄ aryl. Exemplary aryl groups include, but are not limited to, phenyl, naphthyl and anthracyl. In certain embodiments, R^{B} is phenyl (C₆ aryl). In certain embodiments, R^{B} is naphthyl (C₁₀ aryl). In certain embodiments, R^{B} is 5-14 membered heteroaryl. In certain embodiments, R^{B} is 5-10 membered heteroaryl. In certain embodiments, R^{B} is 5-6 membered heteroaryl. In certain embodiments, R is a 5,6-bicyclic heteroaryl. In certain embodiments, R is a 6,6-bicyclic heteroaryl. In certain embodiments, R^{B} is a 5-membered heteroaryl group. Exemplary 5-membered heteroaryl groups include, but are not limited to, pyrrolyl, furanyl, thiophenyl, imidazolyl, pyrazolyl, oxazolyl, isoxazolyl, thiazolyl, isothiazolyl, triazolyl, oxadiazolyl, thiadiazolyl and tetrazolyl. In certain embodiments, R^{B} is a 6-membered heteroaryl group. Exemplary 6-membered heteroaryl groups include, but are not limited to, pyridinyl, pyridazinyl, pyrimidinyl, pyrazinyl, triazinyl and tetrazinyl. In certain embodiments, R^{B} is a 5,6-bicyclic heteroaryl group. Exemplary 5,6-bicyclic heteroaryl groups include, but are not limited to, indolyl, isoindolyl, indazolyl, benztriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. In certain embodiments, R^{B} is a 6,6-bicyclic heteroaryl group. Exemplary 6,6-bicyclic heteroaryl groups include, but are not limited to, naphthyridinyl, pteridinyl, quinolinyl, isoquinolinyl, cinnolinyl, quinoxalinyl, phthalazinyl and quinazolinyl.

In certain embodiments, R^{B} is substituted with the group -L-R^{D} wherein L is a covalent bond or a divalent C₁₋₁₀ hydrocarbon chain, wherein one, two or three methylene units of L are optionally and independently replaced with one or more —O—, —S—, —NR^{B8}—, —(C═NR^{B8})—, —C(═O)—, —C(═S)—, —S(═O)—, —S(═O)₂-divalent C₃₋₁₀ carbocyclyl, divalent 3-14 membered heterocyclyl, divalent C₆₋₁₄ aryl or divalent 5-14 membered heteroaryl group; and R^{D} is selected from —CN, —NO₂, —N₃, —SO₂H, —SO₃H, —C(═O)R^{B7}, —CO₂H, —CHO, —C(OR^{B9})₂, —CO₂R^{B7}, —OC(═O)R^{B7}, —OCO₂R^{B7}, —C(═O)NR^{B8}₂, —OC(═O)NR^{B8}₂, —NR^{B8}C(═O)R^{B7}, —NR^{B8}CO₂R^{B7}, —NR^{B8}C(═O)NR^{B8}₂, —C(═NR^{B8})OR^{B7}, —OC(═NR^{B8})R^{B7}, —OC(═NR^{B8})OR^{B7}, —C(═NR^{B8})NR^{B8}₂, —OC(═NR^{B8})NR^{B8}₂, —NR^{B8}C(═NR^{B8})NR^{B8}₂, —C(═O)NR^{B8}SO₂R^{B7}, —NR^{B8}SO₂R^{B7}, —SO₂NR^{B8}₂, —SO₂R^{B7}, —SO₂OR^{B7}, —OSO₂R^{B7}, —S(═O)R^{B7}, —OS(═O)R^{B7}, —C(═S)NR^{B8}₂, —C(═O)SR^{B7}, —C(═S)SR^{B7}, —SC(═S)SR^{B7}, —P(═O)₂R^{B7}, —OP(═O)₂R^{B7}, —P(═O)(R^{B7})₂, —OP(═O)(R^{B7})₂, —OP(═O)(OR^{B9})₂, —P(═O)₂NR^{B8}₂, OP(═O)₂NR^{B8}₂, —P(═O)(NR^{B8})₂, —OP(═O)(NR^{B8})₂, —NR^{B8}P(═O)(OR^{B9})₂, —NR^{B8}P(═O)(NR^{B8})₂, —B(OR^{B9})₂, —BR^{B7}(OR^{B9}), and tetrazolyl; each instance of R^{B7} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄aryl, and 5-14 membered heteroaryl; each instance of R^{B8} is, independently, selected from hydrogen, —OH, —OR^{B7}, —NR^{B9}₂, —CN, —C(═O)R^{B7}, —C(═O)NR^{B9}₂, —CO₂R^{B7}, —SO₂R^{B7}, C(═NR^{B9})OR^{B7}, —C(═NR^{B9})NR^{B9}₂, —SO₂NR^{B9}₂, —SO₂R^{B9}, —SO₂OR^{B9}, —SOR^{B7}, —C(═S)NR^{B9}₂, —C(═O)SR^{B9}, —C(═S)SR^{B9}, —P(═O)₂R^{B7}, —P(═O)(R^{B7})₂, —P(═O)₂NR^{B9}₂, —P(═O)(NR^{B9})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B8} groups are joined to form a 3-14 membered heterocyclyl or a 5-14 membered heteroaryl ring; and each instance of R^{B9} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B9} groups are joined to form a 3-14 membered heterocyclyl or a 5-14 membered heteroaryl ring.

In certain embodiments, L is a covalent bond. In certain embodiments, L is a divalent C₁₋₁₀ hydrocarbon chain, wherein one, two or three methylene units of L are optionally and independently replaced with one or more —O—, —S—, —NR^{B8}—, —(C═NR^{B8})—, —C(═O)—, —C(═S)—, —S(═O)—, —S(═O)₂— divalent carbocyclyl, divalent heterocyclyl, divalent aryl or divalent heteroaryl group. In certain embodiments, L is a divalent C₁₋₁₀ hydrocarbon chain, wherein one, two or three methylene units of L are optionally and independently replaced with one or more —O—, —S—, —NR^{B8}—, —(C═NR^{B8})—, —C(═O)—, —C(═S)—, —S(═O)—, —S(═O)₂-divalent C₃₋₁₀ carbocyclyl, divalent 3-14 membered heterocyclyl, divalent C₆₋₁₄ aryl or divalent 5-14 membered heteroaryl group.

As generally described above, R^{D} is selected from the group consisting of —CN,—NO₂, —SO₂H, —SO₃H, —C(═O)R^{B7}, —CO₂H, —CHO, —C(OR^{B9})₂, —CO₂R^{B7}, —OC(═O)R^{B7}, —OCO₂R^{B7}, —C(═O)NR^{B8}₂, —OC(═O)NR^{B8}₂, —NR^{B8}C(═O)R^{B7}, —NR^{B8}CO₂R^{B7}, —NR^{B8}C(═O)NR^{B8}₂, —C(═NR^{B8})OR^{B7}, —OC(═NR^{B8})R^{B7}, —OC(═NR^{B8})OR^{B7}, —C(═NR^{B8})NR^{B8}₂, —OC(═NR^{B8})NR^{B8}₂, —NR^{B8}C(═NR^{B8})NR^{B8}₂, —C(═O)NR^{B8}SO₂R^{B7}, —NR^{B8}SO₂R^{B7}, —SO₂NR^{B8}₂, —SO₂R^{B7}, —SO₂OR^{B7}, —OSO₂R^{B7}, —S(═O)R^{B7}, —OS(═O)R^{B7}, —C(═S)NR^{B8}₂, —C(═O)SR^{B7}, —C(═S)SR^{B7}, —SC(═S)SR^{B7}, —P(═O)₂R^{B7}, —OP(═O)₂R^{B7}, —P(═O)(R^{B7})₂, —OP(═O)(R^{B7})₂, —OP(═O)(OR^{B9})₂, —P(═O)₂NR^{B8}₂, —OP(═O)₂NR^{B8}₂, —P(═O)(NR^{B8})₂, —OP(═O)(NR^{B8})₂, —NR^{B8}P(═O)(OR^{B9})₂, —NR^{B8}P(═O)(NR^{B8})₂, —B(OR^{B9})₂, —BR^{B7}(OR^{B9}) and tetrazolyl. However, in certain embodiments, R^{D} is not —CO₂R^{B7} (e.g., CO₂Me, CO₂Et, CO₂nPr, CO₂iPr, or CO₂tBu), but can be selected from any of the other substituents listed above. In certain embodiments, R^{D} is not —C(═O)R^{B7}), but can be selected from any of the other substituents listed above. In certain embodiments, R^{D} is not —CHO), but can be selected from any of the other substituents listed above. In certain embodiments, R^{D} is not —C(OR^{B9})₂), but can be selected from any of the other substituents listed above. In certain embodiments, R^{D} is not —CN), but can be selected from any of the other substituents listed above. In certain embodiments, R^{D} is not —NO₂), but can be selected from any of the other substituents listed above. In certain embodiments, R^{D} is not any one of —SO₂H, —SO₃H, —SO₂NR^{B8}₂, —NR^{B8}SO₂R^{B7}, —SO₂R^{B7}, —SO₂OR^{B7}, —OSO₂R^{B7}, —S(═O)R^{B7} or —OS(═O)R^{B7}), but can be selected from any of the other substituents listed above. In certain embodiments, R^{D} is not any one of —OC(═O)R^{B7}, —OCO₂R^{B7}, —OC(═O)NR^{B8}₂, —NR^{B8}C(═O)R^{B7}, —NR^{B8}CO₂R^{B7}, —NR^{B8}C(═O)NR^{B8}₂, —OC(═NR^{B8})R^{B7}, —OC(═NR^{B8})OR^{B7}, —OC(═NR^{B8})NR^{B8}₂ or —NC(═NR^{B8})NR^{B8}₂, but can be selected from any of the other substituents listed above. In certain embodiments, R^{D} is not any one of —C(═S)NR^{B8}₂, —C(═O)SR^{B7}, —C(═S)SR^{B7} or —SC(═S)SR^{B7}), but can be selected from any of the other substituents listed above. In certain embodiments, R^{D} is not any one of —P(═O)₂R^{B7}, —OP(═O)₂R^{B7}, —P(═O)(R^{B7})₂, —OP(═O)(R^{B7})₂, —OP(═O)(OR^{B9})₂, —P(═O)₂NR^{B8}₂, —OP(═O)₂NR^{B8}₂, —P(═O)(NR^{B8})₂, —OP(═O)(NR^{B8})₂, —NR^{B8}P(═O)(OR^{B9})₂ or —NR^{B8}P(═O)(NR^{B8})₂), but can be selected from any of the other substituents listed above. In certain embodiments, R^{D} is not any one of —B(OR^{B9})₂ or —BR^{B7}(OR^{B9})), but can be selected from any of the other substituents listed above. In certain embodiments, R^{D} is not tetrazolyl), but can be selected from any of the other substituents listed above.

In certain embodiments, R^{D} is selected from —CN, —NO₂, —SO₂H, —SO₃H, —C(═O)R^{B7}, —CO₂H, —CHO, —CO₂R^{B7}, —C(═O)NR^{B8}₂, —C(═NR^{B8})OR^{B7}, —C(═NR^{B8})NR^{B8}₂, —C(═O)NR^{B8}SO₂R^{B7}, —SO₂NR^{B8}₂, —SO₂R^{B7}, —SO₂OR^{B7}, —S(═O)R^{B7}, —C(═S)NR^{B8}₂, —C(═O)SR^{B7}, —C(═S)SR^{B7}, —P(═O)₂R^{B7}, —P(═O)(R^{B7})₂, P(═O)₂NR^{B8}₂, —P(═O)(NR^{B8})₂, —B(OR^{B9})₂, —BR^{B7}(OR^{B9}) and tetrazolyl. In certain embodiments, L is a covalent bond. In certain embodiments, R^{D} is selected from —C(═O)R^{B7}, —CO₂H, —CHO,—CO₂R^{B7}, —C(═O)NR^{B8}₂, —C(═NR^{B8})OR^{B7}, —C(═NR^{B8})NR^{B8}₂, —C(═O)NR^{B8}SO₂R^{B7}, —C(═S)NR^{B8}₂, —C(═O)SR^{B7} and —C(═S)SR^{B7}. In certain embodiments, L is a covalent bond.

In certain embodiments, R^{D} is selected from —C(═O)R^{B7}, —CO₂H, —CHO, and —CO₂R^{B7}. In certain embodiments, L is a covalent bond. In certain embodiments, R^{D} is —CO₂H. In certain embodiments, L is a covalent bond.

In certain embodiments, wherein R^{B} is substituted with -L-R^{D}, R^{B} is further substituted with the group —R^{E} wherein: R^{E} is selected from halogen, —OH, —OR^{B10}, —ONR^{B11}₂, —NR^{B11}₂, —N(OR^{B12})R^{B12}, —SH, —SR^{B10}, —SSR^{B12}, —OC(═O)R^{B10}, —OCO₂R^{B10}, —OC(═O)NR^{B11}₂, —NR^{B11}C(═O)R^{B10}, —NR^{B11}CO₂R^{B10}, NR^{B11} C(═O)NR^{B11}₂, —OC(═NR^{B11})R^{B10}, —OC(═NR^{B11})OR^{B10}, —OC(═NR^{B11})NR^{B11}₂, NR^{B11}C(═NR^{B11})NR^{B11}₂, —NR^{B11}SO₂R^{B10}, —OSO₂R^{B10}, —OS(═O)R^{B10}, —Si(R^{B10})₃, —OSi(R^{B10})₃, —SC(S)SR^{B10}, —OP(═O)₂R^{B10}, —OP(═O)(R^{B10})₂, —OP(═O)(OR^{B12})₂, —OP(═O)₂NR^{B12}, —OP(═O)(NR^{B11})₂, NR^{B11}P(═O)(OR^{B12})₂, —NR^{B11}P(═O)(NR^{B11})₂, —P(R^{B12})₂, —P(R^{B12})₃, —OP(R^{B12})₂, —OP(R^{B12})₃, 3-14 membered heterocyclyl and 5-14 membered heteroaryl, wherein the point of attachment of the 3-14 membered heterocyclyl or 5-14 membered heteroaryl group is on a nitrogen atom; each instance of R^{B10} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each instance of R^{B11} is, independently, selected from hydrogen, —OH, —OR^{B10}, —NR^{B12}₂, —CN, —C(═O)R^{B10}, —C(═O)NR^{B12}₂, —CO₂R^{B10}, —SO₂R^{B10}, —C(═NR^{B12})OR^{B10}, —C(═NR^{B12})NR^{B12}₂, —SO₂NR^{B12}₂, —SO₂R^{B12}, —SO₂OR^{B12}, —SOR^{B10}, —C(═S)NR^{B12}₂, —C(═O)SR^{B12}, —C(═S)SR^{B12}, —P(═O)(R^{B10})₂, —P(═O)₂NR^{B12}₂, —P(═O)(NR^{B12})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B11} groups are joined to form a 3-14 membered heterocyclyl or 5-14 membered heteroaryl ring; and each instance of R^{B12} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B12} groups are joined to form a 3-14 membered heterocyclyl or 5-14 membered heteroaryl ring. In certain embodiments, R^{E} is selected from halogen, —OH, —OR^{B10}, —ONR^{B11}₂, —NR^{B11}₂, —N(OR^{B12})R^{B12}, —SH, —SR^{B10}, —SSR^{B12}, —Si(R^{B10})₃, —OSi(R^{B10})₃, —P(R^{B12})₂, —P(R^{B12})₃, —OP(R^{B12})₂, —OP(R^{B12})₃, 3-14 membered heterocyclyl and 5-14 membered heteroaryl, wherein the point of attachment of the 3-14 membered heterocyclyl or 5-14 membered heteroaryl group is on a nitrogen atom. In certain embodiments, R^{E} is selected from halogen, —OH, —OR^{B10}, —NR^{B11}₂, 3-14 membered heterocyclyl and 5-14 membered heteroaryl, wherein the point of attachment of the 3-14 membered heterocyclyl or 5-14 membered heteroaryl group is on a nitrogen atom. In certain embodiments, R^{E} is selected from halogen, —OR^{B10} and —NR^{B11}₂. In certain embodiments, R^{E} is halogen. In certain embodiments, R^{E} is —OR^{B10}. In certain embodiments, R^{E} is —NR^{B11}₂.

In certain embodiments, -L-R^{D} and —R^{E} are vicinal R^{B} substituents (i.e., attached to two adjacent atoms on the group R^{B}; e.g., ortho to each other). In certain embodiments, -L-R^{D} and —R^{E} are ortho to each other. In certain embodiments, -L-R^{D} and —R^{E} are not vicinal R^{B} substituents (i.e., not attached to two adjacent atoms on the group R^{B}; e.g., meta or para to each other). In certain embodiments, -L-R^{D} and —R^{E} are meta to each other. In certain embodiments, -L-R^{D} and —R^{E} are para to each other.

In certain embodiments, the R^{B} is a group of the formula (vii)

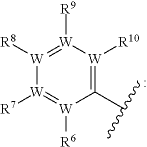

wherein each group W—R⁶, W—R⁷, W—R⁸, W—R⁹, and W—R¹⁰ independently represents either a nitrogen atom (N) or C—R⁶, C—R⁷, C—R⁸, C—R⁹, or C—R¹⁰, respectively; and wherein R⁶, R⁷, R⁸, R⁹ and R¹⁰ are independently selected from the group consisting of hydrogen, halogen, —CN, —NO₂, —N₃, —SO₂H, —SO₃H, —OH, —OR^{B1}, —ONR^{B2}₂, —NR^{B2}₂, —N(OR^{B3})R^{B3}, —SH, —SR^{B1}, —SSR^{B3}, —C(═O)R^{B1}, —CO₂H, —CHO, —C(OR^{B3})₂, —CO₂R^{B1}, —OC(═O)R^{B1}, —OCO₂R^{B1}, —C(═O)NR^{B2}₂, —OC(═O)NR^{B2}₂, —NR^{B2}C(═O)R^{B1}, —NR^{B2}CO₂R^{B1}, —NR^{B2}C(═O)NR^{B2}₂, —C(═NR^{B2})OR^{B1}, —OC(═NR^{B2})R^{B1}, —OC(═NR^{B2})OR^{B1}, —C(═NR^{B2})NR^{B2}₂, —OC(═NR^{B2})NR^{B2}₂, —NR^{B2}C(═NR^{B2})NR^{B2}₂, —C(═O)NR^{B2}SO₂R^{B1}, —NR^{B2}SO₂R^{B1}, —SO₂NR^{B2}₂, —SO₂R^{B1}, —SO₂OR^{B1}, —OSO₂R^{B1}, —S(═O)R^{B1}, —OS(═O)R^{B1}, —Si(R^{B1})₃, —OSi(R^{B1})₃—C(═S)NR^{B2}₂, —C(═O)SR^{B1}, —C(═S)SR^{B1}, —SC(S)SR^{B1}, —P(═O)₂R^{B1}, —OP(═O)₂R^{B1}, —P(═O)(R^{B1})₂, —OP(═O)(R^{B1})₂, —OP(═O)(OR^{B3})₂, —P(═O)₂NR^{B2}₂, —OP(═O)₂NR^{B2}₂, —P(═O)(NR^{B2})₂, —OP(═O)(NR^{B2})₂, —NR^{B2}P(═O)(OR^{B3})₂, —NR^{B2}P(═O)(NR^{B2})₂, —P(R^{B3})₂, —P(R^{B3})₃, —OP(R^{B3})₂, —OP(R^{B3})₃, —B(OR^{B3})₂, or —BR^{B1}(OR^{B3}), C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, 5-14 membered heteroaryl, -L-R^{D} and —R^{E}; or one or more of R⁶ and R⁷, R⁷ and R⁸, R⁸ and R⁹, or R⁹ and R¹⁰ are joined to form a C₃₋₁₀ carbocyclyl, 3-14 membered heterocyclyl, C₆₋₁₄ aryl or 5-14 membered heteroaryl ring; or R and R are joined to form a 3-14 membered heterocyclyl or 5-14 membered heteroaryl ring; each instance of R^{B1} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each instance of R^{B2} is, independently, selected from hydrogen, —OH, —OR^{B1}, —NR^{B3}₂, —CN, —C(═O)R^{B1}, —C(═O)NR^{B3}₂, —CO₂R^{B1}, —SO₂R^{B1}, —C(═NR^{B3})OR^{B1}, —C(═NR^{B3})NR^{B3}₂, —SO₂NR^{B3}₂, —SO₂R^{B3}, —SO₂OR^{B3}, —SOR^{B1}, —C(═S)NR^{B3}₂, —C(═O)SR^{B3}, —C(═S)SR^{B3}, —P(═O)₂R^{B1}, —P(═O)(R^{B1})₂, —P(═O)₂NR^{B3}₂, —P(═O)(NR^{B3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B2} groups are joined to form a 3-14 membered heterocyclyl or 5-14 membered heteroaryl ring; each instance of R^{B3} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B3} groups are joined to form a 3-14 membered heterocyclyl or 5-14 membered heteroaryl ring; and L, R^{D} and R^{E} are as defined above and herein.

As used herein, when one or more of R⁶, R⁷, R⁸, R⁹ and R¹⁰ is referred to as “not hydrogen”, it is meant that one or more of R⁶, R⁷, R⁸, R⁹ and R¹⁰ is independently selected from the group consisting of halogen, —CN, —NO₂, —N₃, —SO₂H, —SO₃H, —OH, —OR^{B1}, —ONR^{B2}₂, —NR^{B2}₂, —N(OR^{B3})R^{B3}, —SH, —SR^{B1}, —SSR^{B3}, —C(═O)R^{B1}, —CO₂H, —CHO, —C(OR^{B3})₂, —CO₂R^{B1}, —OC(═O)R^{B1}, —OCO₂R^{B1}, —C(═O)NR^{B2}₂, —OC(═O)NR^{B2}₂, —NR^{B2}C(═O)R^{B1}, —NR^{B2}CO₂R^{B1}, —NR^{B2}C(═O)NR^{B2}₂, —C(═NR^{B2})OR^{B1}, —OC(═NR^{B2})R^{B1}, —OC(═NR^{B2})OR^{B1}, —C(═NR^{B2})NR^{B2}₂, —OC(═NR^{B2})NR^{B2}₂, NR^{B2}C(═NR^{B2})NR^{B2}₂, —C(═O)NR^{B2}SO₂R^{B1}, —NR^{B2}SO₂R^{B1}, —SO₂NR^{B2}₂, —SO₂R^{B1}, —SO₂OR^{B1}, —OSO₂R^{B1}, —S(═O)R^{B1}, —OS(═O)R^{B1}, —Si(R^{B1})₃, —OSi(R^{B1})₃—C(═S)NR^{B2}₂, —C(═O)SR^{B1}, —C(═S)SR^{B1}, —SC(═S)SR^{B1}, —P(═O)₂R^{B1}, —OP(═O)₂R^{B1}, —P(═O)(R^{B1})₂, —OP(═O)(R^{B1})₂, —OP(═O)(OR^{B3})₂, —P(═O)₂NR^{B2}₂, —OP(═O)₂NR^{B2}₂, —P(═O)(NR^{B2})₂, —OP(═O)(NR^{B2})₂, NR^{B2}P(═O)(OR^{B3})₂, —NR^{B2}P(═O)(NR^{B2})₂, —P(R^{B3})₂, —P(R^{B3})₃, —OP(R^{B3})₂, —OP(R^{B3})₃, —B(OR^{B3})₂, —BR^{B1}(OR^{B3}), -L-R^{D}, —R^{E}, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; or wherein one or more of R⁶ and R⁷, R⁷ and R⁸, R⁸ and R⁹ or R⁹ and R¹⁰ are joined to form a C₃₋₁₀ carbocyclyl, 3-14 membered heterocyclyl, C₆₋₁₄ aryl or 5-14 membered heteroaryl ring, or wherein R¹⁰ and R^{C} are joined to form a 3-14 membered heterocyclyl or 5-14 membered heteroaryl ring.

In certain embodiments, at least one of R⁶, R⁷, R⁸, R⁹, and R¹⁰ is the group as defined above and herein. In certain embodiments, at least one of R⁶, R⁷, R⁸, R⁹, and R¹⁰ is the group —R^{E} as defined herein. In certain embodiments, the group of formula (vii) represents a C₆₋₁₄ aryl or a 6-14 membered heteroaryl group. In certain embodiments, the group of formula (vii) represents a 6-14 membered heteroaryl group. In certain embodiments, the group of formula (vii) represents a C₆₋₁₄ aryl group. In certain embodiments, the group of formula (vii) represents a phenyl group. In certain embodiments, W—R⁶, W—R⁷, W—R⁸, W—R⁹, and W—R¹⁰ represent C—R⁶, C—R⁷, C—R⁸, C—R⁹, or C—R¹⁰, respectively. For example, in certain embodiments, R^{B} is a group of the formula (viii)

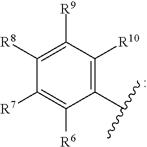

wherein R⁶, R⁷, R⁸, R⁹ and R¹⁰ are as defined above and herein.

In certain embodiments, at least one of R⁶, R⁷, R⁸, R⁹, and R¹⁰ is the group as defined above and herein. In certain embodiments, at least one of R⁶, R⁷, R⁸, R⁹, and R¹⁰ is the group —R^{E} as defined herein. In certain embodiments, the group of the formula (viii) represents a C₆₋₁₄ aryl group. In certain embodiments, the C₆₋₁₄ aryl group of the formula (viii) represents a phenyl group. In certain embodiments, R^{B} is a monosubstituted, disubstituted or trisubstituted group of the formula (viii). In certain embodiments, R^{B} is a monosubstituted or disubstituted group of the formula (viii). In certain embodiments, R^{B} is a monosubstituted group of the formula (viii). For example, in certain embodiments, R^{B} is an ortho-substituted group of formula (viii), e.g., wherein R⁶-R⁹ are hydrogen, and R¹⁰ is not hydrogen. In certain embodiments, R^{B} is a meta-substituted group of the formula (viii), e.g., wherein R⁶-R⁸ and R¹⁰ are hydrogen and R⁹ is not hydrogen. In certain embodiments, R^{B} is a para-substituted group of the formula (viii), e.g., wherein R⁶, R⁷, R⁹ and R¹⁰ are hydrogen and R⁸ is not hydrogen. In certain embodiments, R is a disubstituted group of the formula (viii). For example, in certain embodiments, R^{B} is a 2,6-disubstituted group of the formula (viii), e.g., wherein R⁷, R⁸ and R⁹ are hydrogen, and R⁶ and R¹⁰ are not hydrogen. In certain embodiments, R^{B} is a 2,5-disubstituted group of the formula (viii), e.g., wherein R⁶, R⁸ and R⁹ are hydrogen, and R⁷ and R¹⁰ are not hydrogen. In certain embodiments, R^{B} is a 2,4-disubstituted group of the formula (viii), e.g., wherein R⁶, R⁷ and R⁹ are hydrogen, and R⁸ and R¹⁰ are not hydrogen. In certain embodiments, R^{B} is a 2,3-disubstituted group of formula (viii), e.g., wherein R⁶, R⁷ and R⁸ are hydrogen, and R⁹ and R¹⁰ are not hydrogen. In certain embodiments, R^{B} is a 3,4-disubstituted group of the formula (viii), e.g., wherein R⁶, R⁷ and R¹⁰ are hydrogen, and R⁸ and R⁹ are not hydrogen. In certain embodiments, R^{B} is a 3,5-disubstituted group of the formula (viii), e.g., wherein R¹, R⁴ and R⁵ are hydrogen, and R and R are not hydrogen. In certain embodiments, R is a trisubstituted group of the formula (viii). For example, in certain embodiments, R^{B} is a 2,4, 6-trisubstituted group of formula (viii), e.g., wherein R⁷ and R⁹ are hydrogen, and R⁶, R⁸ and R¹⁰ are not hydrogen. In certain embodiments, R^{B} is a 2,3,6-trisubstituted group of the formula (viii), e.g., wherein R² and R³ are hydrogen, and R¹, R⁴ and R⁵ are not hydrogen. In certain embodiments, R^{B} is a 2,4,5-trisubstituted group of the formula (viii), e.g., wherein R⁸ and R⁹ are hydrogen, and R⁶, R⁷ and R¹⁰ are not hydrogen. In certain embodiments, R^{B} is a 2,3,4-trisubstituted group of the formula (viii), e.g., wherein R⁶ and R⁹ are hydrogen, and R⁷, R⁸ and R¹⁰ are not hydrogen. In certain embodiments, R^{B} is a 3,4,5-trisubstituted group of the formula (viii), e.g., wherein R⁶ and R¹⁰ are hydrogen, and R⁷, R⁸ and R⁹ are not hydrogen.

In certain embodiments, R^{B} is heteroaryl selected from a 5-6-membered heteroaryl, a 5,6-bicyclic heteroaryl, or a 6,6-bicyclic heteroaryl. In certain embodiments, R is a 6-membered heteroaryl. In certain embodiments, R^{A} is a 6-membered heteroaryl selected from pyridinyl. In certain embodiments, R^{B} is 2-pyridinyl, 3-pyridinyl or 4-pyridinyl. In certain embodiments, R^{B} is a 2-pyridinyl wherein W—R⁶ is N, and W—R⁷, W—R⁸, W—R⁹, and W—R¹⁰ are C—R⁷, C—R⁸, C—R⁹ and C—R¹⁰, respectively, e.g., of the formula (ix)

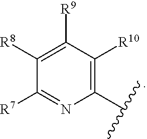

In certain embodiments, R^{B} is a 3-pyridinyl wherein W—R⁷ is N, and W—R⁶, W—R⁸, W—R⁹, and W—R¹⁰ are C—R⁶, C—R⁸, C—R⁹ and C—R¹⁰, respectively, e.g., of the formula (x)

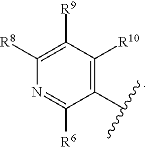

In certain embodiments, R^{B} is a 4-pyridinyl wherein W—R⁸ is N, and W—R⁶, W—R⁷, W—R⁹, and W—R¹⁰ are C—R⁶, C—R⁷, C—R⁹ and C—R¹⁰, respectively, e.g., of the formula (xi)

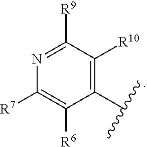

In certain embodiments, at least one of R⁶, R⁷, R⁸, R⁹, and R¹⁰ is the group as defined above and herein. In certain embodiments, at least one of R⁶, R⁷, R⁸, R⁹, and R¹⁰ is the group —R^{E} as defined herein. In certain embodiments, R^{B} is a monosubstituted or disubstituted pyridinyl. In certain embodiments, R^{B} is a monosubstituted pyridinyl. In certain embodiments, R^{B} is a monosubstituted pyridinyl of the formula (ix) wherein R⁸, R⁹, R¹⁰ are hydrogen and R⁷ is not hydrogen. In certain embodiments, R^{B} is a monosubstituted pyridinyl of the formula (ix) wherein R⁷, R⁹, R¹⁰ are hydrogen and R⁸ is not hydrogen. In certain embodiments, R^{B} is a monosubstituted pyridinyl of the formula (ix) wherein R⁷, R⁸, R¹⁰ are hydrogen and R⁹ is not hydrogen. In certain embodiments, R^{B} is a monosubstituted pyridinyl of the formula (ix) wherein R⁷, R⁸, R⁹ are hydrogen and R¹⁰ is not hydrogen. In certain embodiments, R is a monosubstituted pyridinyl of the formula (x) wherein R⁸, R⁹, R¹⁰ are hydrogen and R⁶ is not hydrogen. In certain embodiments, R^{B} is a monosubstituted pyridinyl of the formula (x) wherein R⁶, R⁹, R¹⁰ are hydrogen and R⁸ is not hydrogen. In certain embodiments, R^{B} is a monosubstituted pyridinyl of the formula (x) wherein R⁶, R⁸, R¹⁰ are hydrogen and R⁹ is not hydrogen. In certain embodiments, R^{B} is a monosubstituted pyridinyl of the formula (x) wherein R⁶, R⁸, R⁹ are hydrogen and R¹⁰ is not hydrogen. In certain embodiments, R is a monosubstituted pyridinyl of the formula (xi) wherein R⁶, R⁷, R⁹ are hydrogen and R¹⁰ is not hydrogen. In certain embodiments, R^{B} is a monosubstituted pyridinyl of the formula (v) wherein R⁶, R⁷, R¹⁰ are hydrogen and R⁹ is not hydrogen. In certain embodiments, R is a disubstituted pyridinyl. In certain embodiments, R^{B} is a disubstituted pyridinyl of the formula (ix) wherein R⁸ and R⁹ are hydrogen and R⁷ and R¹⁰ are not hydrogen. In certain embodiments, R^{B} is a disubstituted pyridinyl of the formula (ix) wherein R⁷ and R⁹ are hydrogen and R⁸ and R¹⁰ are not hydrogen. In certain embodiments, R is a disubstituted pyridinyl of the formula (ix) wherein R⁷ and R⁸ are hydrogen and R⁹ and R¹⁰ are not hydrogen. In certain embodiments, R^{B} is a disubstituted pyridinyl of the formula (ix) wherein R⁸ and R¹⁰ are hydrogen and R⁷ and R⁹ are not hydrogen. In certain embodiments, R^{B} is a disubstituted pyridinyl of the formula (ix) wherein R⁹ and R¹⁰ are hydrogen and R⁷ and R⁸ are not hydrogen. In certain embodiments, R^{B} is a disubstituted pyridinyl of the formula (ix) wherein R⁷ and R¹⁰ are hydrogen and R⁸ and R⁹ are not hydrogen. In certain embodiments, R is a disubstituted pyridinyl of the formula (x) wherein R⁸ and R⁹ are hydrogen and R⁶ and R are not hydrogen. In certain embodiments, R^{B} is a disubstituted pyridinyl of the formula (x) wherein R⁸ and R¹⁰ are hydrogen and R⁶ and R⁹ are not hydrogen. In certain embodiments, R^{B} is a disubstituted pyridinyl of the formula (x) wherein R⁹ and R¹⁰ are hydrogen and R⁶ and R⁸ are not hydrogen. In certain embodiments, R^{B} is a disubstituted pyridinyl of the formula (x) wherein R⁶ and R⁹ are hydrogen and R⁸ and R are not hydrogen. In certain embodiments, R^{B} is a disubstituted pyridinyl of the formula (x) wherein R⁶ and R¹⁰ are hydrogen and R⁸ and R⁹ are not hydrogen. In certain embodiments, R is a disubstituted pyridinyl of the formula (x) wherein R⁶ and R⁸ are hydrogen and R⁹ and R¹⁰ are not hydrogen. In certain embodiments, R^{B} is a disubstituted pyridinyl of the formula (xi) wherein R⁷ and R⁹ are hydrogen and R⁶ and R¹⁰ are not hydrogen. In certain embodiments, R^{B} is a disubstituted pyridinyl of the formula (xi) wherein R⁶ and R⁷ are hydrogen and R⁹ and R¹⁰ are not hydrogen. In certain embodiments, R^{B} is a disubstituted pyridinyl of the formula (xi) wherein R⁶ and R⁸ are hydrogen and R⁷ and R¹⁰ are not hydrogen. In certain embodiments, R^{B} is a disubstituted pyridinyl of the formula (xi) wherein R⁶ and R¹⁰ are hydrogen and R⁷ and R⁹ are not hydrogen.

In certain embodiments, R is C₅₋₁₀ carbocyclyl or 5-10 membered heterocyclyl of

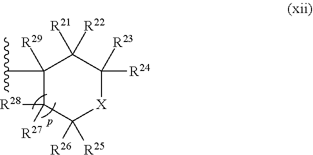

wherein: X is N, NR³⁰, O, S or CR³¹R³²; p is 0, 1 or 2; each R²¹, R²², R²³, R²⁴, R²⁵, R²⁶, R²⁷, R²⁸, R²⁹, R³¹ and R³² is independently selected from hydrogen, halogen, —CN, —NO₂, —N₃, —SO₂H, —SO₃H, —OH, —OR^{B1}, —ONR^{B2}₂, —NR^{B2}₂, —N(OR^{B3})R^{B3}, —SH, —SR^{B1}, —SSR^{B3}, —C(═O)R^{B1}, —CO₂H, —CHO, —C(OR^{B3})₂, —CO₂R^{B1}, —OC(═O)R^{B1}, —OCO₂R^{B1}, —C(═O)NR^{B2}₂, —OC(═O)NR^{B2}₂, —NR^{B2}C(═O)R^{B1}, —NR^{B2}CO₂R^{B1}, —NR^{B2}C(═O)NR^{B2}₂, —C(═NR^{B2})OR^{B1}, —OC(═NR^{B2})R^{B1}, —OC(═NR^{B2})OR^{B1}, —C(═NR^{B2})NR^{B2}₂, —OC(═NR^{B2})NR^{B2}₂, —NR^{B2}C(═NR^{B2})NR^{B2}₂, —C(═O)NR^{B2}SO₂R^{B1}, —NR^{B2}SO₂R^{B1}, —SO₂NR^{B2}₂, —SO₂R^{B1}, —SO₂OR^{B1}, —OSO₂R^{B1}, —S(═O)R^{B1}, —OS(═O)R^{B1}, —Si(R^{B1})₃, —OSi(R^{B1})₃, —C(═S)NR^{B2}₂, —C(═O)SR^{B1}, —C(═S)SR^{B1}, —SC(═S)SR^{B1}, —P(═O)₂R^{B1}, —OP(═O)₂R^{B1}, —P(═O)(R^{B1})₂, —OP(═O)(R^{B1})₂, —OP(═O)(OR^{B3})₂, —P(═O)₂NR^{B2}₂, —OP(═O)₂NR^{B2}₂, —P(═O)(NR^{B2})₂, —OP(═O)(NR^{B2})₂, —NR^{B2}P(═O)(OR^{B3})₂, NR^{B2}P(═O)(NR^{B2})₂, —P(R^{B3})₂, —P(R^{B3})₃, —OP(R^{B3})₂, —OP(R^{B3})₃, —B(OR^{B3})₂, or —BR^{B1}(OR^{B3}), C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, 5-14 membered heteroaryl, -L-R^{D} and —R^{E}; or one or more of R²⁹ and R²¹, R²² and R²³, R²⁴ and R³¹, R³² and R²⁵, R²⁶ and R²⁷, R²⁸ and R²⁹, or R²⁶ and R²⁹, are joined to form a double bond or a C₃₋₁₀ carbocyclyl, 3-14 membered heterocyclyl, C₆₋₁₄ aryl or 5-14 membered heteroaryl ring; optionally wherein X is N, then N and R²³ or N and R²⁵ are joined to form a double bond; R³⁰ is selected from hydrogen, —OH, —OR^{B1}, —NR^{B3}₂, —CN, —C(═O)R^{B1}, —C(═O)NR^{B3}₂, —CO₂R^{B1}, —SO₂R^{B1}, —C(═NR^{B3})OR^{B1}, —C(═NR^{B3})NR^{B3}₂, —SO₂NR^{B3}₂, —SO₂R^{B3}, —SO₂OR^{B3}, —S(═O)R^{B1}, —C(═S)NR^{B3}₂, —C(═O)SR^{B3}, —C(═S)SR^{B3}, —P(═O)₂R^{B1}, —P(═O)(R^{B1})₂, —P(═O)₂NR^{B3}₂, —P(═O)(NR^{B3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, optionally wherein R²⁴ and R³⁰ or R³⁰ and R²⁵ are joined to form a 3-14 membered heterocyclyl or 5-14 membered heteroaryl ring; wherein: each R^{B1} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each R^{B2} is, independently, selected from hydrogen, —OH, —OR^{B1}, —NR^{B3}₂, —CN, —C(═O)R^{B1}, —C(═O)NR^{B3}₂, —CO₂R^{B1}, —SO₂R^{B1}, —C(═NR^{B3})OR^{B1}, —C(═NR^{B3})NR^{B3}₂, —SO₂NR^{B3}₂, —SO₂R^{B3}, —SO₂OR^{B3}, —SOR^{B1}, —C(═S)NR^{B3}₂, —C(═O)SR^{B3}, —C(═S)SR^{B3}, —P(═O)₂R^{B1}, —P(═O)(R^{B1})₂, —P(═O)₂NR^{B3}₂, —P(═O)(NR^{B3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B2} groups are joined to form a 3-14 membered heterocyclyl or 5-14 membered heteroaryl ring; each R^{B3} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B3} groups are joined to form a 3-14 membered heterocyclyl or 5-14 membered heteroaryl ring; and L, R^{D} and R^{E} are as defined above and herein.

In certain embodiments, at least one of R²¹, R²², R²³, R²⁴, R²⁵, R²⁶, R²⁷, R²⁸, R²⁹, R³⁰, R³¹, and R³² is the group -L-R^{D} as defined above and herein. In certain embodiments, at least one of R²¹, R²², R²³, R²⁴, R²⁵, R²⁶, R²⁷, R²⁸, R²⁹, R³⁰, R³¹, and R³² is selected from the group —R^{E} as defined herein. In certain embodiments, p is 0. In certain embodiments, p is 1. In certain embodiments, p is 2. In certain embodiments, X is N. In certain embodiments, X is NR³⁰. In certain embodiments, X is O. In certain embodiments, X is S. In certain embodiments, X is CR³¹R³².

In certain embodiments, R^{B} is C₅₋₁₀ carbocyclyl or 5-10 membered heterocyclyl of the formula (xiii)

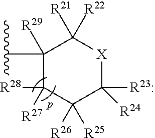

wherein X is N, NR³⁰, O, S or CR³¹R³²; p is 0, 1 or 2; each R²¹, R²², R²³, R²⁴, R²⁵, R²⁶, R²⁷, R²⁸, R²⁹, R³¹ and R³² is independently selected from hydrogen, halogen, —CN, —NO₂, —N₃, —SO₂H, —SO₃H, —OH, —OR^{B1}, —ONR^{B2}₂, —NR^{B2}₂, —N(OR^{B3})R^{B3}, —SH, —SR^{B1}, —SSR^{B3}, —C(═O)R^{B1}, —CO₂H, —CHO, C(OR^{B3})₂, —CO₂R^{B1}, —OC(═O)R^{B1}, —OCO₂R^{B1}, —C(═O)NR^{B2}₂, —OC(═O)NR^{B2}₂, —NR^{B2}C(═O)R^{B1}, —NR^{B2}CO₂R^{B1}, —NR^{B2}C(═O)NR^{B2}₂, —C(═NR^{B2})OR^{B1}, —OC(═NR^{B2})R^{B1}, —OC(═NR^{B2})OR^{B1}, —C(═NR^{B2})NR^{B2}₂, —OC(═NR^{B2})NR^{B2}₂, —NR^{B2}C(═NR^{B2})NR^{B2}₂, —C(═O)NR^{B2}SO₂R^{B1}, —NR^{B2}SO₂R^{B1}, —SO₂NR^{B2}₂, —SO₂R^{B1}, —SO₂OR^{B1}, —OSO₂R^{B1}, —S(═O)R^{B1}, —OS(═O)R^{B1}, —Si(R^{B1})₃, —OSi(R^{B1})₃—C(═S)NR^{B2}₂, —C(═O)SR^{B1}, —C(═S)SR^{B1}, —SC(═S)SR^{B1}, —P(═O)₂R^{B1}, —OP(═O)₂R^{B1}, —P(═O)(R^{B1})₂, —OP(═O)(R^{B1})₂, —OP(═O)(OR^{B3})₂, —P(═O)₂NR^{B2}₂, —OP(═O)₂NR^{B2}₂, —P(═O)(NR^{B2})₂, —OP(═O)(NR^{B2})₂, —NR^{B2}P(═O)(OR^{B3})₂, NR^{B2}P(═O)(NR^{B2})₂, —P(R^{B3})₂, —P(R^{B3})₃, —OP(R^{B3})₂, —OP(R^{B3})₃, —B(OR^{B3})₂, or —BR^{B1}(OR^{B3}), C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, 5-14 membered heteroaryl, -L-R^{D} and —R^{E}; or one or more of R²⁹ and R²¹, R²² and R³¹, R³² and R²³, R²⁴ and R²⁵, R²⁶ and R²⁷, R²⁸ and R²⁹, and R²⁶ and R²⁹, are joined to form a double bond or a C₃₋₁₀ carbocyclyl, 3-14 membered heterocyclyl, C₆₋₁₄ aryl or 5-14 membered heteroaryl ring; optionally wherein X is N, then N and R²¹ or N and R²³ are joined to form a double bond; R³⁰ is selected from hydrogen, —OH, —OR^{B1}, —NR^{B3}₂, —CN, —C(═O)R^{B1}, —C(═O)NR^{B3}₂, —CO₂R^{B1}, —SO₂R^{B1}, —C(═NR^{B3})OR^{B1}, —C(═NR^{B3})NR^{B3}₂, —SO₂NR^{B3}₂, —SO₂R^{B3}, —SO₂OR^{B3}, —S(═O)R^{B1}, —C(═S)NR^{B3}₂, —C(═O)SR^{B3}, —C(═S)SR^{B3}, —P(═O)₂R^{B1}, —P(═O)(R^{B1})₂, —P(═O)₂NR^{B3}₂, —P(═O)(NR^{B3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or R²² and R³⁰ or R³⁰ and R²³ are joined to form a 3-14 membered heterocyclyl or 5-14 membered heteroaryl ring; wherein: each R^{B1} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl; each R^{B2} is, independently, selected from hydrogen, —OH, —OR^{B1}, —NR^{B3}₂, —CN, —C(═O)R^{B1}, —C(═O)NR^{B3}₂, —CO₂R^{B1}, —SO₂R^{B1}, —C(═NR^{B3})OR^{B1}, —C(═NR^{B3})NR^{B3}₂, —SO₂NR^{B3}₂, —SO₂R^{B3}, —SO₂OR^{B3}, —S(═O)R^{B1}, —C(═S)NR^{B3}₂, —C(═O)SR^{B3}, —C(═S)SR^{B3}, —P(═O)₂R^{B1}, —O(═O)(R^{B1})₂, P(═O)₂NR^{B3}₂, —P(═O)(NR^{B3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B2} groups are joined to form a 3-14 membered heterocyclyl or 5-14 membered heteroaryl ring; each R^{B3} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{B3} groups are joined to form a 3-14 membered heterocyclyl or 5-14 membered heteroaryl ring; and L, R^{D} and R^{E} are as defined above and herein.

In certain embodiments, at least one of R²¹, R²², R²³, R²⁴, R²⁵, R²⁶, R²⁷, R²⁸, R²⁹, R³⁰, R³¹, and R³² is the group -L-R^{D} as defined above and herein. In certain embodiments, at least one of at least one of R²¹, R²², R²³, R²⁴, R²⁵, R²⁶, R²⁷, R²⁸, R²⁹, R³⁰, R³¹, and R³² is selected from —R^{E} as defined herein. In certain embodiments, p is 0. In certain embodiments, p is 1. In certain embodiments, p is 2. In certain embodiments, p is 2. In certain embodiments, X is N. In certain embodiments, X is NR³⁰. In certain embodiments, X is O. In certain embodiments, X is S. In certain embodiments, X is CR³¹R³². For example, in certain embodiments, X is O.

In certain embodiments, R^{B} is a 5-10 membered heterocyclyl of the formulae

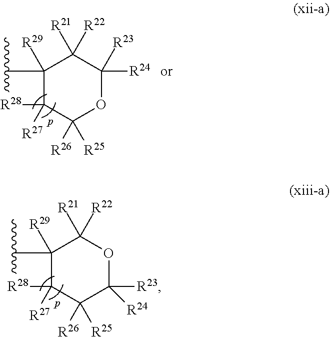

wherein p, R²¹, R²², R²³, R²⁴, R²⁵, R²⁶, R²⁷, R²⁸ and R²⁹ are as defined above and herein.

In certain embodiments, X is NR³⁰. For example, in certain embodiments, R^{B} is heterocyclyl of the formulae

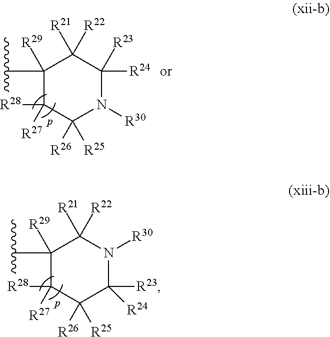

wherein p, R²¹, R²², R²³, R²⁴, R²⁵, R²⁶, R²⁷, R²⁸, R²⁹ and R³⁰ are as defined above and herein.

In certain embodiments, X is CR³¹R³². For example, in certain embodiments, R¹ is C₅₋₁₀ carbocyclyl of

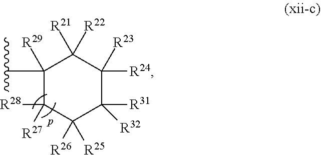

p, R²¹, R²², R²³, R²⁴, R²⁵, R²⁶, R²⁷, R²⁸, R²⁹, R³¹, and R³² are as defined above and herein.

As described generally above, in certain embodiments, R^{B} and R^{C} together with the nitrogen (N) atom to which each is attached are joined to form a 5-14 membered ring.

For example, in certain embodiments, R^{B} and R^{C} together with the nitrogen (N) atom to which each is attached are joined to form a 5-14 membered ring of the formula

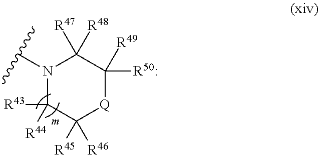

wherein: Q is N, NR⁴⁰, O, S, or CR⁴¹R⁴², M is 0, 1 or 2; and each R⁴¹, R⁴², R⁴³, R⁴⁴, R⁴⁵, R⁴⁶, R⁴⁷, R⁴⁸, R⁴⁹ and R⁵⁰ is independently selected from hydrogen, halogen, —CN, —NO₂, —N₃, —SO₂H, —SO₃H, —OH, —OR^{F1}, —ONR^{F2}₂, —NR^{F2}₂, —N(OR^{F3})R^{F3}, —SH, —SR^{F1}, —SSR^{F3}, —C(═O)R^{F1}, —CO₂H, —CHO, —C(OR^{F3})₂, —CO₂R^{F1}, OC(═O)R^{F1}, —OCO₂R^{F1}, —C(═O)NR^{F2}₂, —OC(═O)NR^{F2}₂, —NR^{F2}C(═O)R^{F1}, —NR^{F2}CO₂R^{F1}, —NR^{F2}C(═O)NR^{F2}₂, —C(═NR^{F2})OR^{F1}, —OC(═NR^{F2})R^{F1}, —OC(═NR^{F2})OR^{F1}, —C(═NR^{F2})NR^{F2}₂, —OC(═NR^{F2})NR^{F2}₂, —NR^{F2}C(═NR^{F2})NR^{F2}₂, —C(═O)NR^{F2}SO₂R^{BC1}, —NR^{F2}SO₂R^{F1}, —SO₂NR^{F2}₂, —SO₂R^{F1}, —SO₂OR^{F1}, —OSO₂R^{F1}, —S(═O)R^{F1}, —OS(═O)R^{F1}, —Si(R^{F1})₃, —OSi(R^{F1})₃₋C(═S)NR^{F2}₂, —C(═O)SR^{F1}, —C(═S)SR^{F1}, —SC(═S)SR^{F1}, P(═O)₂R^{F1}, —OP(═O)₂R^{F1}, —P(═O)(R^{F1})₂, —OP(═O)(R^{F1})₂, —OP(═O)(OR^{F3})₂, —P(═O)₂NR^{F2}₂, —OP(═O)₂NR^{F2}₂, —P(═O)(NR^{F2})₂, —OP(═O)(NR^{F2})₂, —NR^{F2}P(═O)(OR^{F3})₂, —NR^{F2}P(═O)(NR^{F2})₂, —P(R^{F3})₂, —P(R^{F3})₃, —OP(R^{F3})₂, —OP(R^{F3})₃, —B(OR^{F3})₂, or —BR^{F1} (OR^{F3}), C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, 5-14 membered heteroaryl, -L-R^{D} and —R^{E}; or one or more of R⁴⁷ and R⁴⁹, R⁴⁸ and R⁵⁰, R⁴⁹ and R⁴¹, R⁵⁰ and R⁴², R⁴¹ and R⁴⁵, R⁴² and R⁴⁶, R⁴⁵ and R⁴³, and R⁴⁶ and R⁴⁴, are joined to form a double bond or a C₃₋₁₀ carbocyclyl, 3-14 membered heterocyclyl, C₆₋₁₄ aryl or 5-14 membered heteroaryl ring; optionally wherein Q is N, then N and R⁴⁹ or N and R⁴⁶ are joined to form a double bond; R⁴⁰ is selected from hydrogen, —OH, —OR^{F1}, —NR^{F3}₂, —CN, —C(═O)R^{F1}, —C(═O)NR^{F3}₂, —CO₂R^{F1}, —SO₂R^{F1}, —C(═NR^{F3})OR^{F1}, —C(═NR^{F3})NR^{F3}₂, —SO₂NR^{F3}₂, —SO₂R^{F3}, —SO₂OR^{F3}, —SOR^{F1}, —C(═S)NR^{F3}₂, —C(═O)SR^{F3}, —C(═S)SR^{F3}, —P(═O)₂R^{F1}, P(═O)(R^{F1})₂, —P(═O)₂NR^{F3}₂, —P(═O)(NR^{F3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, optionally wherein R⁴⁹ and R⁴⁰ or R⁴⁰ and R⁴⁵ are joined to form a 3-14 membered heterocyclyl, or 5-14 membered heteroaryl ring; each R^{F1} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl; each R^{F2} is, independently, selected from hydrogen, —OH, —OR^{F1}, —NR^{F3}₂, —CN, —C(═O)R^{F1}, —C(═O)NR^{F3}₂, —CO₂R^{F1}, —SO₂R^{F1}, —C(═NR^{F3})OR^{F1}, —C(═NR^{F3})NR^{F3}₂, —SO₂NR^{F3}₂, —SO₂R^{F3}, —SO₂OR^{F3}, —S(═O)R^{F1}, —C(═S)NR^{F3}₂, —C(═O)SR^{F3}, —C(═S)SR^{F3}, —P(═O)₂R^{F1}, —P(═O)(R^{F1})₂, —P(═O)₂NR^{F3}₂, P(═O)(NR^{F3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{F2} groups are joined to form a 3-14 membered heterocyclyl or 5-14 membered heteroaryl ring; each R^{F3} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{F3} groups are joined to form a 3-14 membered heterocyclyl or 5-14 membered heteroaryl ring; and L, R^{D} and R^{E} are as defined above and herein.

In certain embodiments, at least one of R⁴⁰, R⁴¹, R⁴², R⁴³, R⁴⁴, R⁴⁵, R⁴⁶, R⁴⁷, R⁴⁸, R⁴⁹ and R⁵⁰ is the group -L-R^{D} as defined above and herein. In certain embodiments, at least one of R⁴⁰, R⁴¹, R⁴², R⁴³, R⁴⁴, R⁴⁵, R⁴⁶, R⁴⁷, R⁴⁸, R⁴⁹ and R⁵⁰ is selected from —R^{E} as defined herein. In certain embodiments, m is 0. In certain embodiments, m is 1. In certain embodiments, m is 2. In certain embodiments, Q is N. In certain embodiments, Q is NR. In certain embodiments, Q is O. In certain embodiments, Q is S. In certain embodiments, Q is CR⁴¹R⁴². In certain embodiments, R⁴⁷ and R⁴⁹ are joined to form a double bond and R⁴⁸ and R⁵⁰ are joined to form a C₆₋₁₄ aryl or 5-14 membered heteroaryl.

For example, in certain embodiments, R and R together with the nitrogen (N) atom to which each is attached are joined to form a 5-14 membered ring of the formula

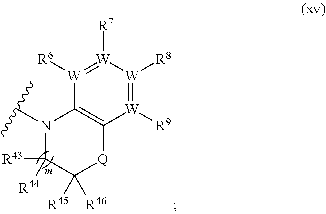

wherein Q, m, R⁴¹, R⁴², R⁴³, R⁴⁴, R⁴⁵, R⁴⁶, R⁶, R⁷, R⁸ and R⁹ are as defined above and herein.

In certain embodiments, Q is CR⁴¹R⁴², R⁴⁹ and R⁴¹ are joined to form a double bond and R⁵⁰ and R⁴² are joined to form a C₆₋₁₄ aryl or 5-14 membered heteroaryl. For example, in certain embodiments, R^{B} and R^{C} together with the nitrogen (N) atom to which each is attached are joined to form a group of the formula (xvi):

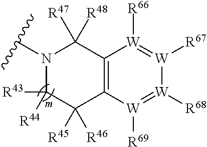

wherein m, R⁴³, R⁴⁴, R⁴⁵, R⁴⁶, R⁴⁷ and R⁴⁸ are as defined above and herein; and wherein R⁶⁶, R⁶⁷, R⁶⁸ and R⁶⁹ are independently selected from hydrogen, halogen, —CN, —NO₂, —N₃, —SO₂H, —SO₃H, —OH, —OR^{F4}, —ONR^{F5}₂, —NR^{F5}₂, —N(OR^{F6})R^{F6}, —SH, —SR^{E4}, —SSR^{F6}, —C(═O)R^{E4}, —CO₂H, —CHO, —C(OR^{F6})₂, —CO₂R^{F4}, —OC(═O)R^{F4}, —OCO₂R^{F4}, —C(═O)NR^{F5}₂, —OC(═O)NR^{F5}₂, —NR^{F5}C(═O)R^{F4}, NR^{F5}CO₂R^{F4}, —NR^{F5}C(═O)NR^{F5}₂, —C(═NR^{F5})OR^{F4}, —OC(═NR^{F5})R^{F4}, —OC(═NR^{F5})OR^{F4}, —C(═NR^{F5})NR^{F5}₂, OC(═NR^{F5)}NR^{F5}₂, —NR^{F5}C(═NR^{F5})NR^{F5}₂, —C(═O)NR^{F5}SO₂R^{F4}, —NR^{F5}SO₂R^{F4}, —SO₂NR^{F5}₂, —SO₂R^{F4} SO₂OR^{F4}, —OSO₂R^{F4}, —S(═O)R^{F4}, —OS(═O)R^{F4}, —Si(R^{F4})₃, —OSi(R^{F4})₃—C(═S)NR^{F5}₂, —C(═O)SR^{F4}, C(═S)SR^{F4}, —SC(S)SR^{F4}, —P(═O)₂R^{F4}, —OP(═O)₂R^{F4}, —P(═O)(R^{F4})₂, —OP(═O)(R^{F4})₂, —OP(═O)(OR^{F6})₂, P(═O)₂NR^{F5}₂, —OP(═O)₂NR^{F5}₂, —P(═O)(NR^{F5})₂, —OP(═O)(NR^{F5})₂, —NR^{F5}P(═O)(OR^{F6})₂, NR^{F5}P(═O)(NR^{F5})₂, —P(R^{F6})₂, —P(R^{F6})₃, —OP(R^{F6})₂, —OP(R^{F6})₃, —B(OR^{F6})₂, or —BR^{F4}(OR^{F6}), C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, 5-14 membered heteroaryl, -L-R^{D} and —R^{E}; or one or more of R⁶⁶ and R⁶⁷, R⁶⁷ and R⁶⁸, and R⁶⁸ and R⁶⁹ are joined to form a C₃₋₁₀ carbocyclyl, 3-14 membered heterocyclyl, C₆₋₁₄ aryl or 5-14 membered heteroaryl ring; each R^{F4} is, independently, selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; each R^{F5} is, independently, selected from hydrogen, —OH, —OR^{F4}, —NR^{F6}₂, —CN, —C(═O)R^{F4}, —C(═O)NR^{F6}₂, —CO₂R^{F4}, —SO₂R^{F4}, —C(═NR^{F6})OR^{F4}, —C(═NR^{F6})NR^{F6}₂, —SO₂NR^{F6}₂, —SO₂R^{F6}, SO₂OR^{F6}, —SOR^{F4}, —C(═S)NR^{F6}₂, —C(═O)SR^{F6}, —C(═S)SR^{F6}, —P(═O)₂R^{F4}, —P(═O)(R^{F4})₂, —P(═O)₂NR^{F6}₂, P(═O)(NR^{F6})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{F5} groups are joined to form a 3-14 membered heterocyclyl or 5-14 membered heteroaryl ring; and each R^{F6} is, independently, selected from hydrogen, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{F6} groups are joined to form a 3-14 membered heterocyclyl or 5-14 membered heteroaryl ring.

In certain embodiments, at least one of R⁴³, R⁴⁴, R⁴⁵, R⁴⁶, R⁴⁷, R⁴⁸, R⁶⁶, R⁶⁷, R⁶⁸ and R⁶⁹ is the group -L-R^{D} as defined above and herein. In certain embodiments, at least one of R⁴³, R⁴⁴, R⁴⁵, R⁴⁶, R⁴⁷, R⁴⁸, R⁶⁶, R⁶⁷, R⁶⁸ and R⁶⁹ is selected from —R^{E} as defined herein. In certain embodiments, m is 0. In certain embodiments, m is 1. In certain embodiments, m is 2.

As described generally above, R is selected from hydrogen, —OH, —OR,—ONR^{C2}₂, —NR^{C2}₂, —C(═O)R^{C1}, —CHO, —CO₂R^{C1}, —C(═O)NR^{C2}₂, —C(═NR^{C2})OR^{C1}, —C(═NR^{C2})NR^{C2}₂, —SO₂R^{C1}, —S(═O)R^{C1}, —Si(R^{C1})₃, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl; wherein: each instance of R^{C1} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl; and each instance of R^{C2} is, independently, selected from hydrogen, —OH, —OR, —NR^{C3}₂, —CN, —C(═O)R^{C1}, —C(═O)NR^{C3}₂, —CO₂R^{C1}, —SO₂R^{C1}, —C(═NR^{C3})OR^{C1}, —C(═NR^{C3})NR^{C3}₂, —SO₂NR^{C3}₂, —SO₂R^{C3}, —SO₂OR^{C3}, —SOR^{C1}, —C(═S)NR^{C3}₂, —C(═O)SR^{C3}, —C(═S)SR^{C3}, —P(═O)₂R¹, —P(═O)(R^{C1})₂, —P(═O)₂NR^{C3}₂, P(═O)(NR^{C3})₂, C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl, or two R^{C2} groups are joined to form a 3-14 membered heterocyclyl or 5-14 membered heteroaryl ring; or R^{B} and R^{C} together with the nitrogen (N) atom to which each is attached are joined to form a 5-14 membered ring. In certain embodiments, R^{C} is selected from C₁₋₁₀ alkyl, C₁₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl. In certain embodiments, R^{C} is an unsubstituted group, e.g., selected from unsubstituted C₁₋₁₀ alkyl, unsubstituted C₂₋₁₀ alkenyl, unsubstituted C₂₋₁₀ alkynyl, unsubstituted 3-14 membered heteroaliphatic, unsubstituted C₃₋₁₀ carbocyclyl, unsubstituted 3-14 membered heterocyclyl, unsubstituted C₆₋₁₄ aryl and unsubstituted 5-14 membered heteroaryl. However, in certain embodiments, R is an unsubstituted group wherein —CH₃ and —CH₂CH₃ are excluded. In certain embodiments, R^{C} is a group having 2 or more carbon atoms, e.g., selected from C₂₋₁₀ alkyl, C₂₋₁₀ perhaloalkyl, C₂₋₁₀ alkenyl, C₂₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl. In certain embodiments, R is an unsubstituted group having 2 or more carbon atoms. However, in certain embodiments, R is a group having 2 or more carbon atoms wherein —CH₂CH₃ is excluded. In certain embodiments, R^{C} is a group having 3 or more carbon atoms, e.g., selected from C₃₋₁₀ alkyl, C₃₋₁₀ perhaloalkyl, C₃₋₁₀ alkenyl, C₃₋₁₀ alkynyl, 3-14 membered heteroaliphatic, C₃₋₁₀ carbocyclyl, 3-14 membered heterocyclyl, C₆₋₁₄ aryl, and 5-14 membered heteroaryl. In certain embodiments, R^{C} is an unsubstituted group having 3 or more carbon atoms. However, in certain embodiments, R^{C} is a group having 3 or more carbon atoms wherein —CH(CH₃)₂ is excluded. In certain embodiments, R^{C} is a group having 4 or more carbon atoms, e.g., selected from C₄₋₁₀ alkyl, C₄₋₁₀ perhaloalkyl, C₄₋₁₀ alkenyl, C₄₋₁₀ alkynyl, 5-14 membered heteroaliphatic, C₅₋₁₀ carbocyclyl, 5-14 membered heterocyclyl, C₆₋₁₄ aryl, and 5-14 membered heteroaryl. In certain embodiments, R is an unsubstituted group having 4 or more carbon atoms.

In certain embodiments, R^{C} is an acyclic group, e.g., selected from C₁₋₁₀ alkyl, C₂₋₁₀ alkenyl, C₂₋₁₀ alkynyl and 3-14 membered heteroaliphatic. In certain embodiments, R^{C} is an unsubstituted acyclic group, e.g., selected from unsubstituted C₁₋₁₀ alkyl, unsubstituted C₂₋₁₀ alkenyl, unsubstituted C₂₋₁₀ alkynyl and unsubstituted 3-14 membered heteroaliphatic. However, in certain embodiments, R is an acyclic group, wherein —CH₃ and —CH₂CH₃ are excluded.

In certain embodiments, R^{C} is C₁₋₁₀ alkyl. In certain embodiments, R^{C} is an unsubstituted C₁₋₁₀ alkyl. In certain embodiments, R^{C} is C₁₋₁₀ alkyl, wherein —CH₃ is excluded. In certain embodiments, R^{C} is C₁₋₁₀ alkyl, wherein —CH₂CH₃ is excluded. In certain embodiments, R^{C} is C₁₋₁₀ alkyl, wherein —CH(CH₃)₂ is excluded. In certain embodiments, R^{C} is C₂₋₁₀ alkyl, e.g., selected from ethyl, n-propyl, isopropyl, n-butyl, tert-butyl, sec-butyl, iso-butyl, n-pentyl, pentan-3-yl, amyl, neopentyl, 3-methyl-2-butanyl, tertiary amyl and n-hexyl. In certain embodiments, R^{C} is an unsubstituted C₂₋₁₀ alkyl. In certain embodiments, R^{C} is C₂₋₁₀ alkyl, wherein —CH₂CH₃ is excluded. In certain embodiments, R^{C} is C₂₋₁₀ alkyl, wherein —CH(CH₃)₂ is excluded. In certain embodiments, R^{C} is C₃₋₁₀ alkyl, e.g., selected from n-propyl, isopropyl, n-butyl, tert-butyl, sec-butyl, iso-butyl, n-pentyl, pentan-3-yl, amyl, neopentyl, 3-methyl-2-butanyl, tertiary amyl and n-hexyl. In certain embodiments, R^{C} is an unsubstituted C₃₋₁₀ alkyl. In certain embodiments, R^{C} is C₃₋₁₀ alkyl, wherein —CH(CH₃)₂ is excluded. In certain embodiments, R^{C} is C₄₋₁₀ alkyl, e.g., selected from n-butyl, tert-butyl, sec-butyl, iso-butyl, n-pentyl, pentan-3-yl, amyl, neopentyl, 3-methyl-2-butanyl, tertiary amyl and n-hexyl. In certain embodiments, R^{C} is an unsubstituted C₄₋₁₀ alkyl. In certain embodiments, R^{C} is C₂₋₁₀ alkenyl. In certain embodiments, R^{C} is an unsubstituted C₂₋₁₀ alkenyl. In certain embodiments, R^{C} is C₂₋₁₀ alkenyl selected from allyl. In certain embodiments, R^{C} is C₂₋₁₀ alkynyl. In certain embodiments, R^{C} is an unsubstituted C₂₋₁₀ alkynyl.

In certain embodiments, R^{C} is 3-14 membered heteroaliphatic. In certain embodiments, R^{C} is an unsubstituted 3-14 membered heteroaliphatic. In certain embodiments, R^{C} is a cyclic group, e.g., selected from C₃₋₁₀ carbocyclyl, 3-14 membered heterocyclyl, C₆₋₁₄ aryl and 5-14 membered heteroaryl. In certain embodiments, R^{C} is an unsubstituted cyclic group, e.g., selected from unsubstituted C₃₋₁₀ carbocyclyl, unsubstituted 3-14 membered heterocyclyl, unsubstituted C₆₋₁₄ aryl and unsubstituted 5-14 membered heteroaryl. In certain embodiments, R^{C} is C₃₋₁₀ carbocyclyl. In certain embodiments, R^{C} is C₄₋₁₀ carbocyclyl. In certain embodiments, R^{C} is C₅₋₁₀ carbocyclyl. In certain embodiments, R^{C} is C₅₋₈ carbocyclyl. In certain embodiments, R^{C} is C₃₋₁₀ carbocyclyl selected from cyclopropyl (C₃), cyclobutyl (C₄), cyclopentyl (C₅), cyclopentenyl (C₅), cyclohexyl (C₆), cyclohexenyl (C₆), cyclohexadienyl (C₆), cycloheptyl (C₇), cycloheptadienyl (C₇), cycloheptatrienyl (C₇) and cyclooctyl (C₈). In certain embodiments, R is C₃₋₁₀ carbocyclyl selected from cyclopentyl and cyclohexyl. In certain embodiments, R is an unsubstituted C₃₋₁₀ carbocyclyl. In certain embodiments, R^{C} is 3-14 membered heterocyclyl. In certain embodiments, R^{C} is 5-10 membered heterocyclyl. In certain embodiments, R^{C} is 5-6 membered heterocyclyl. In certain embodiments, R^{C} is 3-14 membered heterocyclyl selected from azirdinyl, oxiranyl, thiorenyl, azetidinyl, oxetanyl, thietanyl, tetrahydrofuranyl, dihydrofuranyl, tetrahydrothiophenyl, dihydrothiophenyl, pyrrolidinyl, dihydropyrrolyl, dioxolanyl, oxathiolanyl, dithiolanyl, piperidinyl, tetrahydropyranyl, dihydropyridinyl, thianyl, piperazinyl, morpholinyl, dithianyl, dioxanyl, azepanyl, oxepanyl thiepanyl, azocanyl, oxecanyl and thiocanyl. In certain embodiments, R^{C} is 3-14 membered heterocyclyl selected from tetrahydropyranyl. In certain embodiments, R^{C} is an unsubstituted 3-14 membered heterocyclyl. In certain embodiments, R^{C} is C₆₋₁₄ aryl. In certain embodiments, R^{C} is a C₆₋₁₄ aryl selected from phenyl, naphthyl and anthracyl. In certain embodiments, R a C₆₋₁₄ aryl selected from phenyl. In certain embodiments, R^{C} is an unsubstituted C₆₋₁₄ aryl. In certain embodiments, R^{C} is 5-14 membered heteroaryl. In certain embodiments, R^{C} is 5-10 membered heteroaryl. In certain embodiments, R^{C} is 5-6 membered heteroaryl. In certain embodiments, R^{C} is a 5-membered heteroaryl, e.g., selected from pyrrolyl, furanyl, thiophenyl, imidazolyl, pyrazolyl, oxazolyl, isoxazolyl, thiazolyl, isothiazolyl, triazolyl, oxadiazolyl, thiadiazolyl and tetrazolyl. In certain embodiments, R^{A} is a 6-membered heteroaryl, e.g., selected from pyridinyl, pyridazinyl, pyrimidinyl, pyrazinyl, triazinyl and tetrazinyl. In certain embodiments, R^{C} is an unsubstituted 5-14 membered heteroaryl.

In some embodiments, the compound has the structure of Formula (LI):

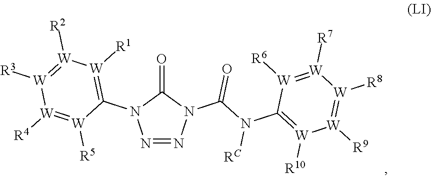

or a pharmaceutically acceptable salt thereof. In some embodiments, at least one of R⁶, R⁷, R⁸, R⁹, and R¹⁰ is the group -L-R^{D}. In some embodiments, at least one of R⁶, R⁷, R⁸, R⁹, and R¹⁰ is the group —R^{E}.

In some embodiments, the compound has the structure of one of the following:

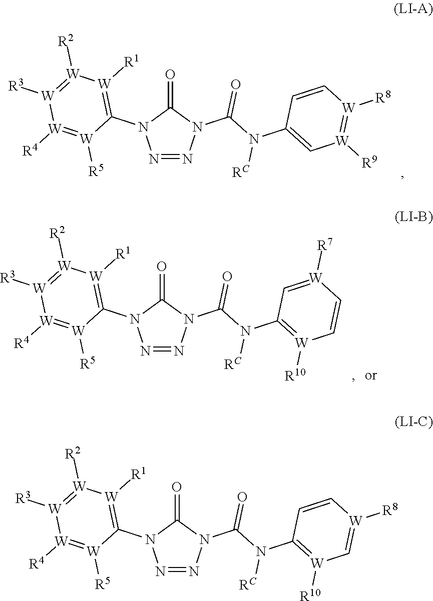

or a pharmaceutically acceptable salt thereof. In some embodiments, at least one of R⁷, R⁸, R⁹, and R¹⁰ is the group -L-R^{D}. In some embodiments, at least one of R⁷, R⁸, R⁹, and R¹⁰ is the group —R^{E}.

In some embodiments, the compound has the structure of Formula (LII):

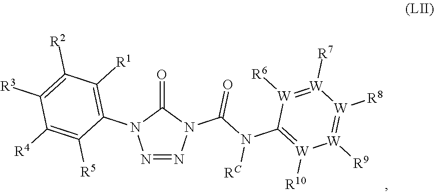

or a pharmaceutically acceptable salt thereof. In some embodiments, at least one of R⁶, R⁷, R⁸, R⁹, and R¹⁰ is the group -L-R^{D}. In some embodiments, at least one of R⁶, R⁷, R⁸, R⁹, and R¹⁰ is the group —R^{E}.

In some embodiments, the compound has the structure of one of the following:

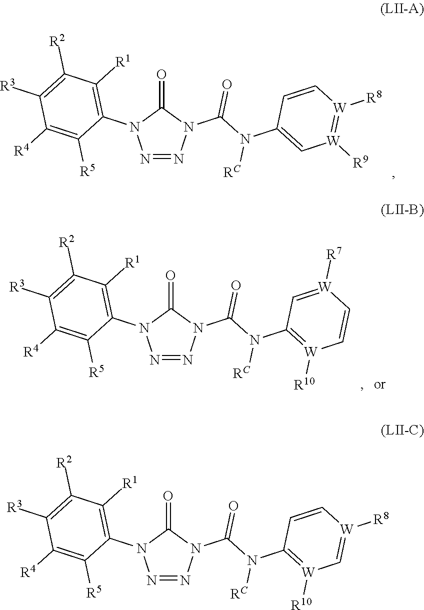

or a pharmaceutically acceptable salt thereof. In some embodiments, at least one of R⁷, R⁸, R⁹, and R¹⁰ is the group -L-R^{D}. In some embodiments, at least one of R⁷, R⁸, R⁹, and R¹⁰ is the group —R^{E}.

In some embodiments, the compound as the structure of Formula (LIII):

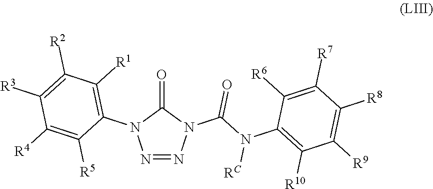

or a pharmaceutically acceptable salt thereof.

In certain embodiments, at least one of R⁶, R⁷, R⁸, R⁹ and R¹⁰ of the formula (LIII) is the group -L-R^{D} as defined above and herein. In certain embodiments, at least one of R⁶, R⁷, R⁸, R⁹ and R¹⁰ of the formula (LIII) is further selected from the group —R^{E} as defined above and herein. In certain embodiments, R¹-R⁵ are independently H, C₁₋₁₀ alkyl, C₁₋₁₀ alkyloxy, C₆₋₁₄ aryloxy, CN, —SO₂NR^{A7}₂, —SO₂R^{A6}, and —SO₂OR^{A6}; R^{C} is unsubstituted C_{M0} alkyl or unsubstituted C₃₋₁₀ carbocyclyl; and R⁶-R¹⁰ are independently selected from H, C₁₋₁₀ alkyl, C₁₋₁₀ alkyloxy, C₆₋₁₄ aryloxy, COOH, and —CO₂R^{A6}. In certain embodiments, R¹-R⁵ are independently H, methyl, methoxy, CN, and SO₂Me; R^{C} is unsubstituted C₁₋₃ alkyl or unsubstituted C₅₋₆ cycloalkyl; and R⁶R¹⁰ are independently selected from H, methyl, methoxy, phenoxy, COOH, and CO₂Me.

In some embodiments, the compound has the structure of one of the following:

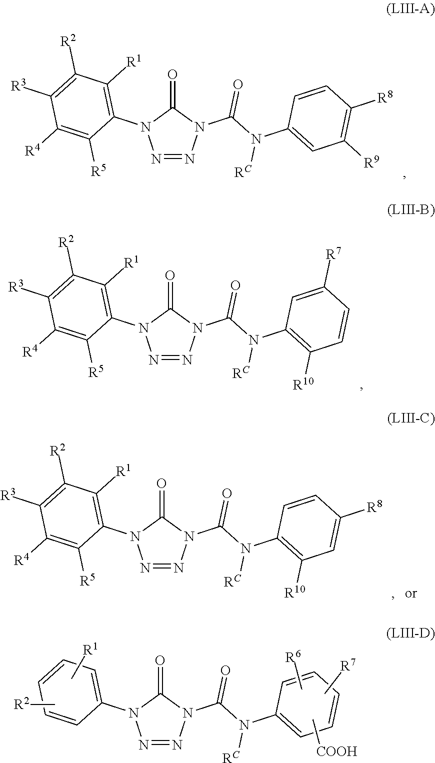

or a pharmaceutically acceptable salt thereof. In certain embodiments, at least one of R⁷, R⁸, R⁹, and R¹⁰ is the group -L-R^{D}. In some embodiments, at least one of R⁷, R⁸, R⁹, and R¹⁰ is the group —R^{E}.

In some embodiments, R^{C} is C₁₋₁₀ alkyl or C₃₋₁₀ carbocyclyl. In some embodiments R^{C} is ethyl, isopropyl, cyclopentyl, or cyclohexyl. In some embodiments, each of R¹ and R² is, independently, hydrogen, halogen, —CN, —OR^{A1}, or —SO₂R^{A1}, wherein R^{A1} is C₁₋₁₀ alkyl. In some embodiments, each of R¹ and R² is, independently, hydrogen, fluoro, methoxy, —CN, or —SO₂CH₃. In some embodiments, each of R₆ and R⁷ is, independently, hydrogen, halogen, or —O—R^{B1}, wherein R^{B1} is C₁₋₁₀ alkyl or C₆₋₁₄ aryl. In some embodiments, each of R₆ and R⁷ is, independently, hydrogen, fluoro, methoxy, or phenyloxy.

In some embodiments, the compound has the structure of Formula (LIV):

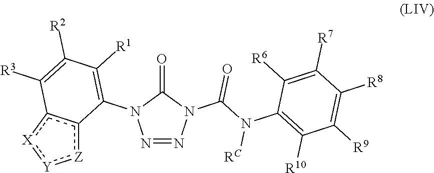

or a pharmaceutically acceptable salt thereof. In some embodiments, at least one of R⁶, R⁷, R⁸, R⁹, and R¹⁰ is the group -L-R^{D}. In some embodiments, at least one of R⁶, R⁷, R⁸, R⁹, and R¹⁰ is the group —R^{E}.

In some embodiments, the compound has the structure of one of the following:

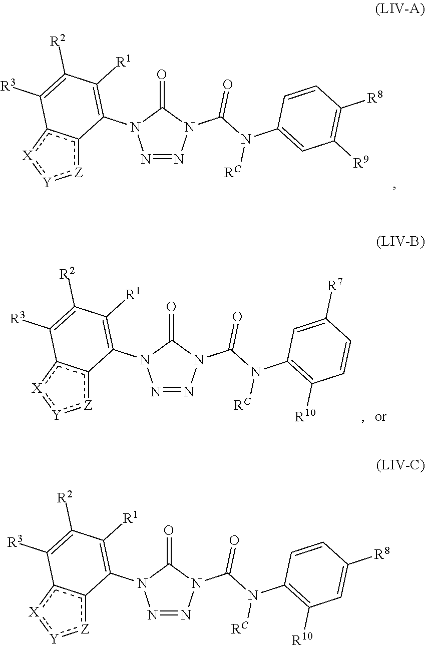

or a pharmaceutically acceptable salt thereof. In some embodiments, at least one of R⁷, R⁸, R⁹, and R¹⁰ is the group -L-R^{D}. In some embodiments, at least one of R⁷, R⁸, R⁹, and R¹⁰ is the group —R^{E}.

In some embodiments, the compound has the structure of Formula (LV):

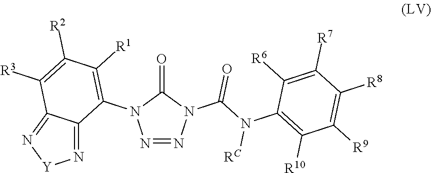

or a pharmaceutically acceptable salt thereof.

In some embodiments, at least one of R⁶, R⁷, R⁸, R⁹, and R¹⁰ is the group -L-R^{D}. In some embodiments, at least one of R⁶, R⁷, R⁸, R⁹, and R¹⁰ is the group —R^{E}. In some embodiments, R¹-R³ are independently H, C₁₋₁₀ alkyl, C₁₋₁₀ alkyloxy, C₆₋₁₄ aryloxy, CN, —SO₂NR^{A7}₂, —SO₂R^{A6}, and —SO₂OR^{A6}; R^{C} is unsubstituted C₁₋₁₀ alkyl or unsubstituted C₃₋₁₀ carbocyclyl; and R⁶-R¹⁰ are independently selected from H, C₁₋₁₀ alkyl, C₁₋₁₀ alkyloxy, C₆₋₁₄ aryloxy, COOH, and —CO₂R^{A6}. In certain embodiments, R¹-R³ are independently H, methyl, methoxy, and CN; R^{C} is unsubstituted C₅₋₆ cycloalkyl; and R⁶-R¹⁰ are independently selected from H, methyl, methoxy, phenoxy, COOH, and CO₂Me.

In some embodiments, the compound has the structure of one of the following:

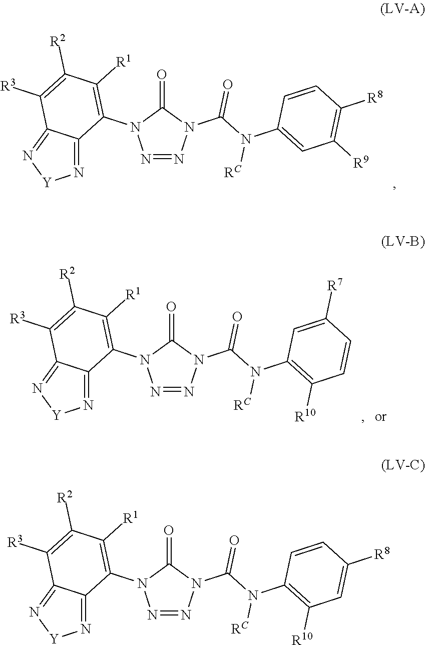

or a pharmaceutically acceptable salt thereof. In some embodiments, at least one of R⁷, R⁸, R⁹, and R¹⁰ is the group -L-R^{D}. In some embodiments, at least one of R⁷, R⁸, R⁹, and R¹⁰ is the group —R^{E}.

In some embodiments, the compound has the structure of one of the following:

| | Compound |
|---|---|
| 2081 | 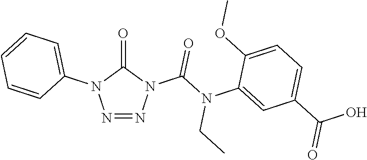 [SMILES: CCN(C(=O)n1nnn(-c2ccccc2)c1=O)c1cc(C(=O)O)ccc1OC] |
| 2082 | 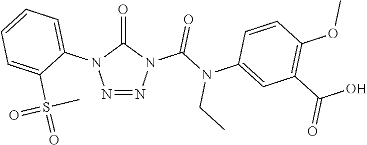 [SMILES: CCN(C(=O)n1nnn(-c2ccccc2S(C)(=O)=O)c1=O)c1ccc(OC)c(C(=O)O)c1] |
| 2083 | 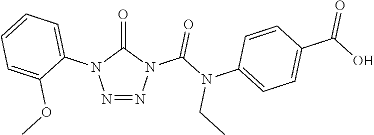 [SMILES: CCN(C(=O)n1nnn(-c2ccccc2OC)c1=O)c1ccc(C(=O)O)cc1] |
| 2084 | 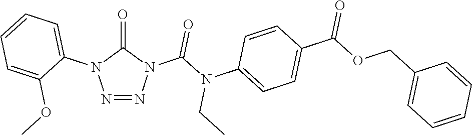 [SMILES: CCN(C(=O)n1nnn(-c2ccccc2OC)c1=O)c1ccc(C(=O)OCc2ccccc2)cc1] |
| 2085 | 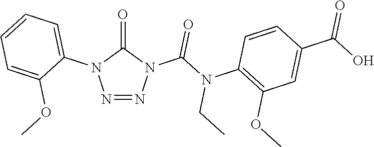 [SMILES: CCN(C(=O)n1nnn(-c2ccccc2OC)c1=O)c1ccc(C(=O)O)cc1OC] |
| 2086 | 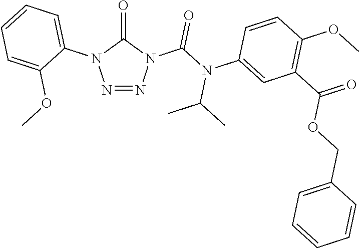 [SMILES: COc1ccc(N(C(=O)n2nnn(-c3ccccc3OC)c2=O)C(C)C)cc1C(=O)OCc1ccccc1] |
| 2087 | 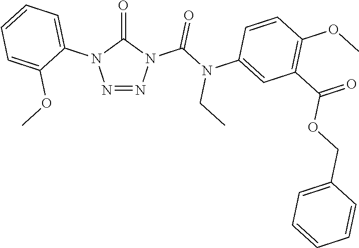 [SMILES: CCN(C(=O)n1nnn(-c2ccccc2OC)c1=O)c1ccc(OC)c(C(=O)OCc2ccccc2)c1] |
| 2088 | 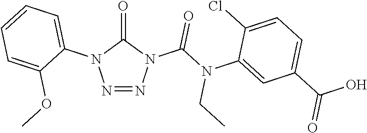 [SMILES: CCN(C(=O)n1nnn(-c2ccccc2OC)c1=O)c1cc(C(=O)O)ccc1Cl] |
| 2089 | 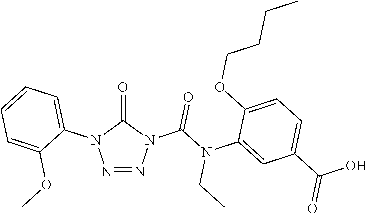 [SMILES: CCCCOc1ccc(C(=O)O)cc1N(CC)C(=O)n1nnn(-c2ccccc2OC)c1=O] |
| 2090 | 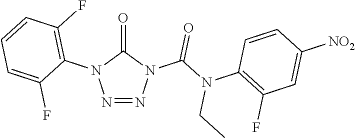 [SMILES: CCN(C(=O)n1nnn(-c2c(F)cccc2F)c1=O)c1ccc([N+](=O)[O-])cc1F] |
| 2091 | 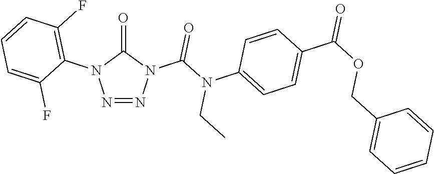 [SMILES: CCN(C(=O)n1nnn(-c2c(F)cccc2F)c1=O)c1ccc(C(=O)OCc2ccccc2)cc1] |
| 2092 | 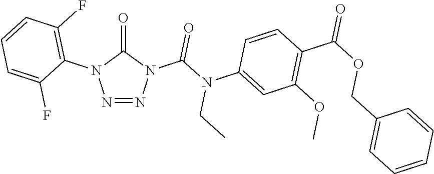 [SMILES: CCN(C(=O)n1nnn(-c2c(F)cccc2F)c1=O)c1ccc(C(=O)OCc2ccccc2)c(OC)c1] |
| 2093 | 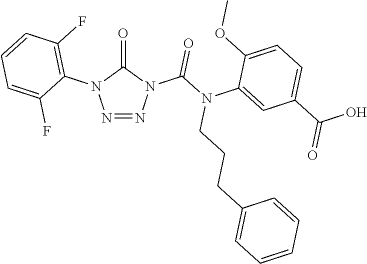 [SMILES: COc1ccc(C(=O)O)cc1N(CCCc1ccccc1)C(=O)n1nnn(-c2c(F)cccc2F)c1=O] |
| 2094 | 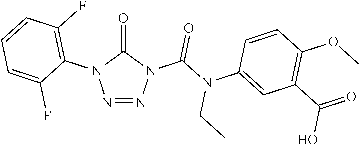 [SMILES: CCN(C(=O)n1nnn(-c2c(F)cccc2F)c1=O)c1ccc(OC)c(C(=O)O)c1] |
| 2095 | 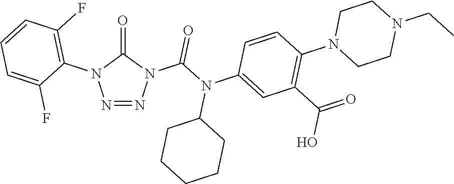 [SMILES: CCN1CCN(c2ccc(N(C(=O)n3nnn(-c4c(F)cccc4F)c3=O)C3CCCCC3)cc2C(=O)O)CC1] |
| 2096 | 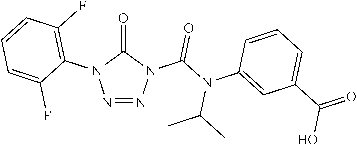 [SMILES: CC(C)N(C(=O)n1nnn(-c2c(F)cccc2F)c1=O)c1cccc(C(=O)O)c1] |
| 2097 | 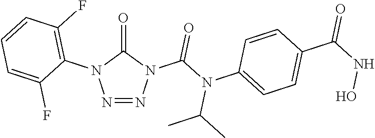 [SMILES: CC(C)N(C(=O)n1nnn(-c2c(F)cccc2F)c1=O)c1ccc(C(=O)NO)cc1] |
| 2098 | 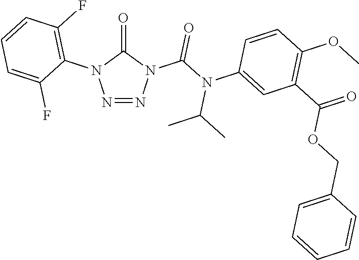 [SMILES: COc1ccc(N(C(=O)n2nnn(-c3c(F)cccc3F)c2=O)C(C)C)cc1C(=O)OCc1ccccc1] |
| 2099 | 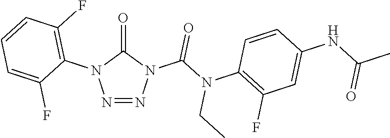 [SMILES: CCN(C(=O)n1nnn(-c2c(F)cccc2F)c1=O)c1ccc(NC(C)=O)cc1F] |
| 2100 | 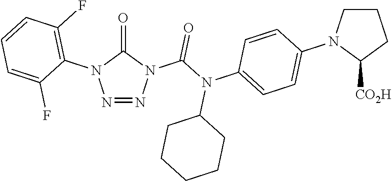 [SMILES: O=C(O)[C@@H]1CCCN1c1ccc(N(C(=O)n2nnn(-c3c(F)cccc3F)c2=O)C2CCCCC2)cc1] |
| 2101 | 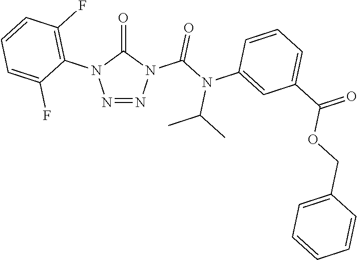 [SMILES: CC(C)N(C(=O)n1nnn(-c2c(F)cccc2F)c1=O)c1cccc(C(=O)OCc2ccccc2)c1] |
| 2102 | 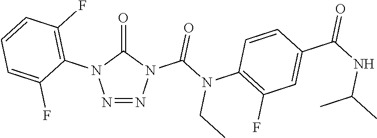 [SMILES: CCN(C(=O)n1nnn(-c2c(F)cccc2F)c1=O)c1ccc(C(=O)NC(C)C)cc1F] |
| 2103 | 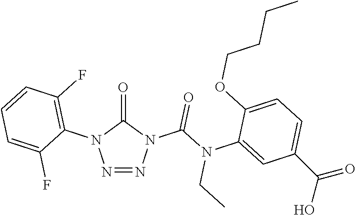 [SMILES: CCCCOc1ccc(C(=O)O)cc1N(CC)C(=O)n1nnn(-c2c(F)cccc2F)c1=O] |
| 2104 | 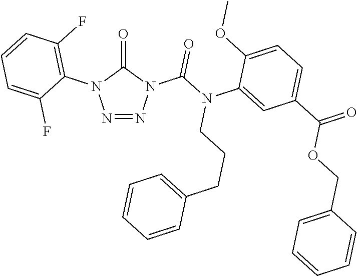 [SMILES: COc1ccc(C(=O)OCc2ccccc2)cc1N(CCCc1ccccc1)C(=O)n1nnn(-c2c(F)cccc2F)c1=O] |
| 2105 | 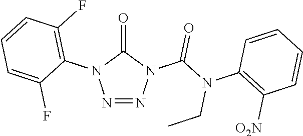 [SMILES: CCN(C(=O)n1nnn(-c2c(F)cccc2F)c1=O)c1ccccc1[N+](=O)[O-]] |
| 2106 | 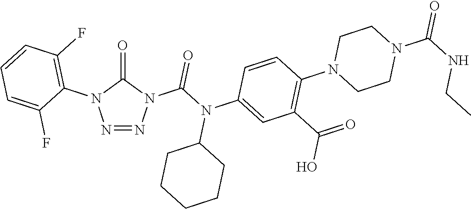 [SMILES: CCNC(=O)N1CCN(c2ccc(N(C(=O)n3nnn(-c4c(F)cccc4F)c3=O)C3CCCCC3)cc2C(=O)O)CC1] |
| 2107 | 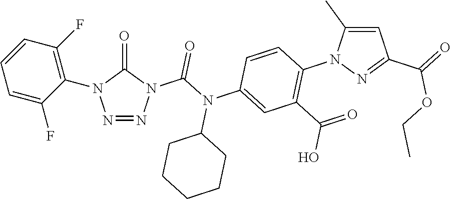 [SMILES: CCOC(=O)c1cc(C)n(-c2ccc(N(C(=O)n3nnn(-c4c(F)cccc4F)c3=O)C3CCCCC3)cc2C(=O)O)n1] |
| 2108 | 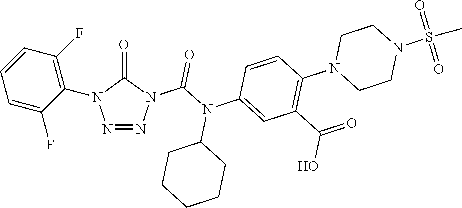 [SMILES: CS(=O)(=O)N1CCN(c2ccc(N(C(=O)n3nnn(-c4c(F)cccc4F)c3=O)C3CCCCC3)cc2C(=O)O)CC1] |
| 2109 | 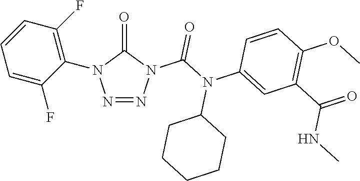 [SMILES: CNC(=O)c1cc(N(C(=O)n2nnn(-c3c(F)cccc3F)c2=O)C2CCCCC2)ccc1OC] |
| 2110 | 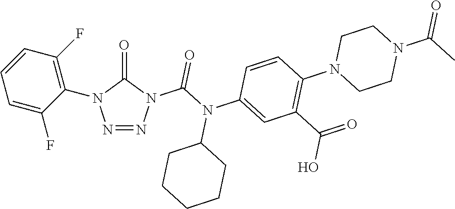 [SMILES: CC(=O)N1CCN(c2ccc(N(C(=O)n3nnn(-c4c(F)cccc4F)c3=O)C3CCCCC3)cc2C(=O)O)CC1] |
| 2111 | 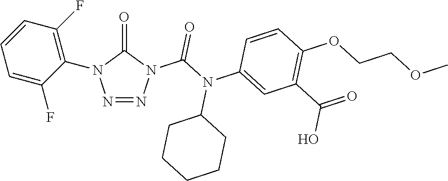 [SMILES: COCCOc1ccc(N(C(=O)n2nnn(-c3c(F)cccc3F)c2=O)C2CCCCC2)cc1C(=O)O] |
| 2112 | 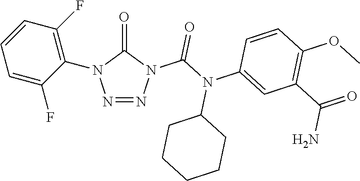 [SMILES: COc1ccc(N(C(=O)n2nnn(-c3c(F)cccc3F)c2=O)C2CCCCC2)cc1C(N)=O] |
| 2113 | 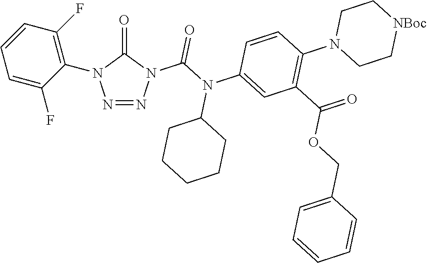 [SMILES: CC(C)(C)OC(=O)N1CCN(c2ccc(N(C(=O)n3nnn(-c4c(F)cccc4F)c3=O)C3CCCCC3)cc2C(=O)OCc2ccccc2)CC1] |
| 2114 | 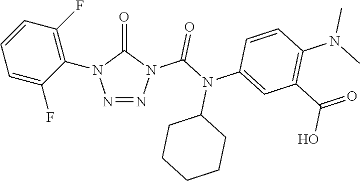 [SMILES: CN(C)c1ccc(N(C(=O)n2nnn(-c3c(F)cccc3F)c2=O)C2CCCCC2)cc1C(=O)O] |
| 2115 | 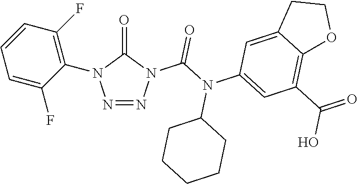 [SMILES: O=C(O)c1cc(N(C(=O)n2nnn(-c3c(F)cccc3F)c2=O)C2CCCCC2)cc2c1OCC2] |
| 2116 | 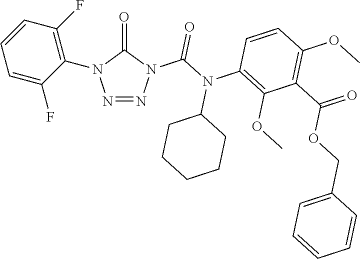 [SMILES: COc1ccc(N(C(=O)n2nnn(-c3c(F)cccc3F)c2=O)C2CCCCC2)c(OC)c1C(=O)OCc1ccccc1] |
| 2117 | 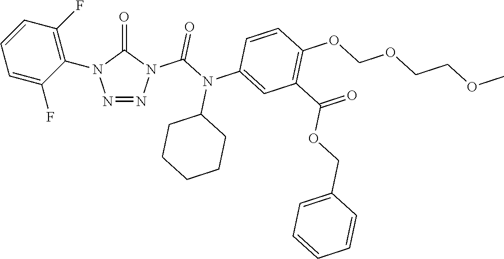 [SMILES: COCCOCOc1ccc(N(C(=O)n2nnn(-c3c(F)cccc3F)c2=O)C2CCCCC2)cc1C(=O)OCc1ccccc1] |
| 2118 | 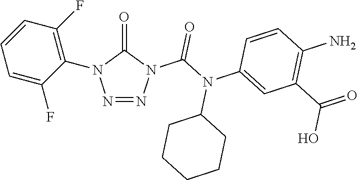 [SMILES: Nc1ccc(N(C(=O)n2nnn(-c3c(F)cccc3F)c2=O)C2CCCCC2)cc1C(=O)O] |
| 2119 | 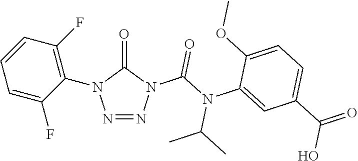 [SMILES: COc1ccc(C(=O)O)cc1N(C(=O)n1nnn(-c2c(F)cccc2F)c1=O)C(C)C] |
| 2120 | 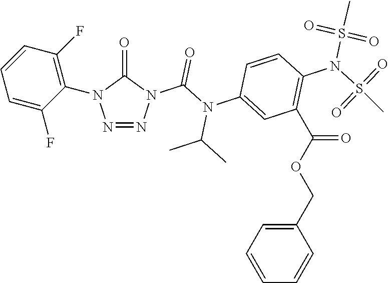 [SMILES: CC(C)N(C(=O)n1nnn(-c2c(F)cccc2F)c1=O)c1ccc(N(S(C)(=O)=O)S(C)(=O)=O)c(C(=O)OCc2ccccc2)c1] |
| 2121 | 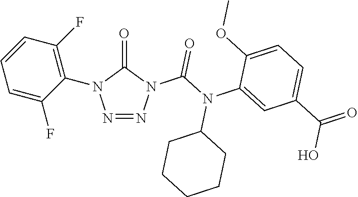 [SMILES: COc1ccc(C(=O)O)cc1N(C(=O)n1nnn(-c2c(F)cccc2F)c1=O)C1CCCCC1] |
| 2122 | 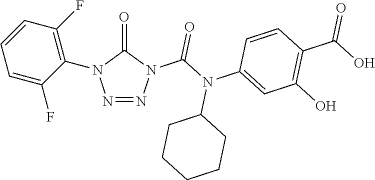 [SMILES: O=C(O)c1ccc(N(C(=O)n2nnn(-c3c(F)cccc3F)c2=O)C2CCCCC2)cc1O] |
| 2123 | 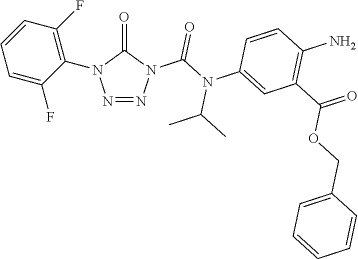 [SMILES: CC(C)N(C(=O)n1nnn(-c2c(F)cccc2F)c1=O)c1ccc(N)c(C(=O)OCc2ccccc2)c1] |
| 2124 | 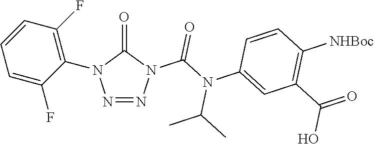 [SMILES: CC(C)N(C(=O)n1nnn(-c2c(F)cccc2F)c1=O)c1ccc(NC(=O)OC(C)(C)C)c(C(=O)O)c1] |
| 2125 | 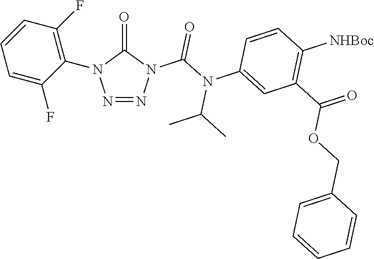 [SMILES: CC(C)N(C(=O)n1nnn(-c2c(F)cccc2F)c1=O)c1ccc(NC(=O)OC(C)(C)C)c(C(=O)OCc2ccccc2)c1] |
| 2126 | 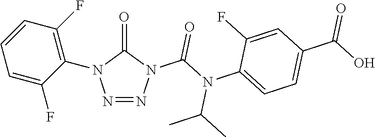 [SMILES: CC(C)N(C(=O)n1nnn(-c2c(F)cccc2F)c1=O)c1ccc(C(=O)O)cc1F] |
| 2127 | 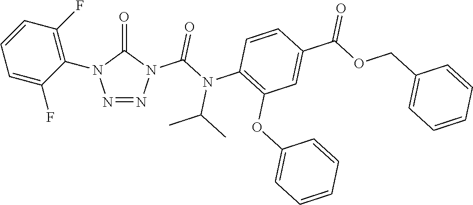 [SMILES: CC(C)N(C(=O)n1nnn(-c2c(F)cccc2F)c1=O)c1ccc(C(=O)OCc2ccccc2)cc1Oc1ccccc1] |
| 2128 | 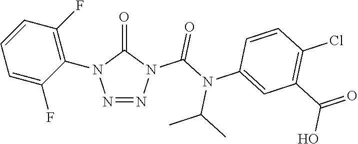 [SMILES: CC(C)N(C(=O)n1nnn(-c2c(F)cccc2F)c1=O)c1ccc(Cl)c(C(=O)O)c1] |
| 2129 | 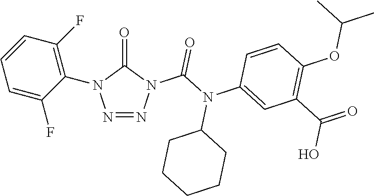 [SMILES: CC(C)Oc1ccc(N(C(=O)n2nnn(-c3c(F)cccc3F)c2=O)C2CCCCC2)cc1C(=O)O] |
| 2130 | 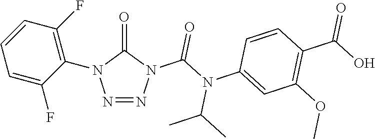 [SMILES: COc1cc(N(C(=O)n2nnn(-c3c(F)cccc3F)c2=O)C(C)C)ccc1C(=O)O] |
| 2131 | 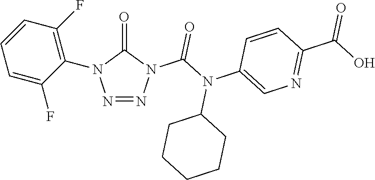 [SMILES: O=C(O)c1ccc(N(C(=O)n2nnn(-c3c(F)cccc3F)c2=O)C2CCCCC2)cn1] |
| 2132 | 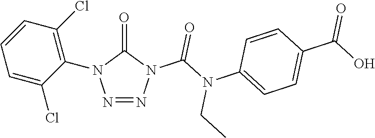 [SMILES: CCN(C(=O)n1nnn(-c2c(Cl)cccc2Cl)c1=O)c1ccc(C(=O)O)cc1] |
| 2133 | 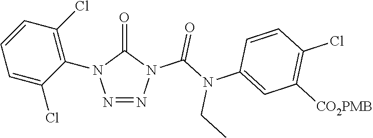 |
| 2134 | 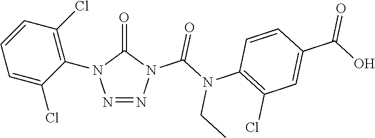 [SMILES: CCN(C(=O)n1nnn(-c2c(Cl)cccc2Cl)c1=O)c1ccc(C(=O)O)cc1Cl] |
| 2135 | 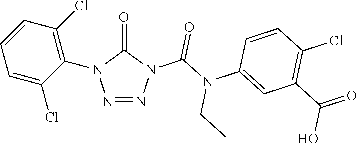 [SMILES: CCN(C(=O)n1nnn(-c2c(Cl)cccc2Cl)c1=O)c1ccc(Cl)c(C(=O)O)c1] |
| 2136 | 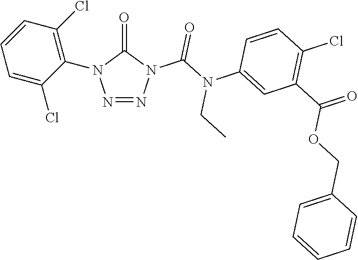 [SMILES: CCN(C(=O)n1nnn(-c2c(Cl)cccc2Cl)c1=O)c1ccc(Cl)c(C(=O)OCc2ccccc2)c1] |
| 2137 | 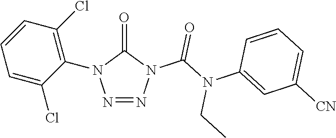 [SMILES: CCN(C(=O)n1nnn(-c2c(Cl)cccc2Cl)c1=O)c1cccc(C#N)c1] |
| 2138 | 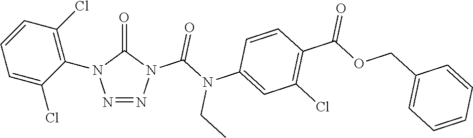 [SMILES: CCN(C(=O)n1nnn(-c2c(Cl)cccc2Cl)c1=O)c1ccc(C(=O)OCc2ccccc2)c(Cl)c1] |
| 2139 | 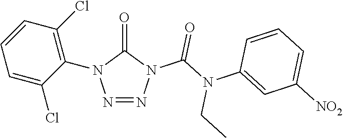 [SMILES: CCN(C(=O)n1nnn(-c2c(Cl)cccc2Cl)c1=O)c1cccc([N+](=O)[O-])c1] |
| 2140 | 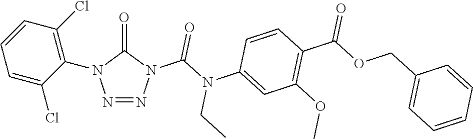 [SMILES: CCN(C(=O)n1nnn(-c2c(Cl)cccc2Cl)c1=O)c1ccc(C(=O)OCc2ccccc2)c(OC)c1] |
| 2141 | 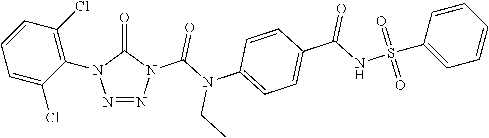 [SMILES: CCN(C(=O)n1nnn(-c2c(Cl)cccc2Cl)c1=O)c1ccc(C(=O)NS(=O)(=O)c2ccccc2)cc1] |
| 2142 | 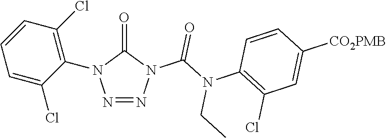 |
| 2143 | 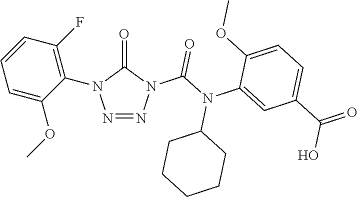 [SMILES: COc1ccc(C(=O)O)cc1N(C(=O)n1nnn(-c2c(F)cccc2OC)c1=O)C1CCCCC1] |
| 2144 | 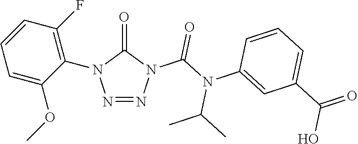 [SMILES: COc1cccc(F)c1-n1nnn(C(=O)N(c2cccc(C(=O)O)c2)C(C)C)c1=O] |
| 2145 | 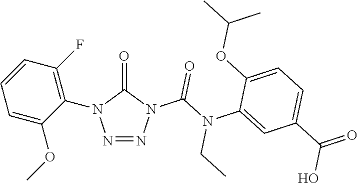 [SMILES: CCN(C(=O)n1nnn(-c2c(F)cccc2OC)c1=O)c1cc(C(=O)O)ccc1OC(C)C] |
| 2146 | 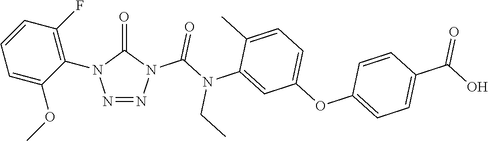 [SMILES: CCN(C(=O)n1nnn(-c2c(F)cccc2OC)c1=O)c1cc(Oc2ccc(C(=O)O)cc2)ccc1C] |
| 2147 | 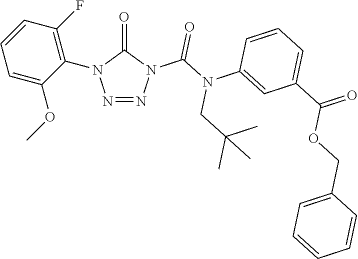 [SMILES: COc1cccc(F)c1-n1nnn(C(=O)N(CC(C)(C)C)c2cccc(C(=O)OCc3ccccc3)c2)c1=O] |
| 2148 | 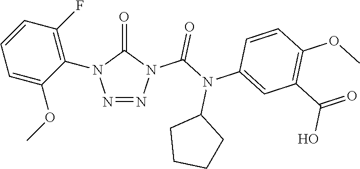 [SMILES: COc1ccc(N(C(=O)n2nnn(-c3c(F)cccc3OC)c2=O)C2CCCC2)cc1C(=O)O] |
| 2149 | 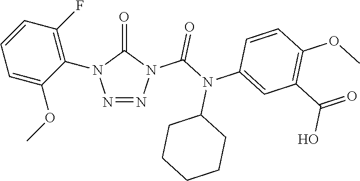 [SMILES: COc1ccc(N(C(=O)n2nnn(-c3c(F)cccc3OC)c2=O)C2CCCCC2)cc1C(=O)O] |
| 2150 | 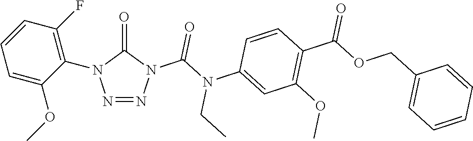 [SMILES: CCN(C(=O)n1nnn(-c2c(F)cccc2OC)c1=O)c1ccc(C(=O)OCc2ccccc2)c(OC)c1] |
| 2151 | 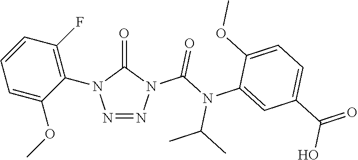 [SMILES: COc1ccc(C(=O)O)cc1N(C(=O)n1nnn(-c2c(F)cccc2OC)c1=O)C(C)C] |
| 2152 | 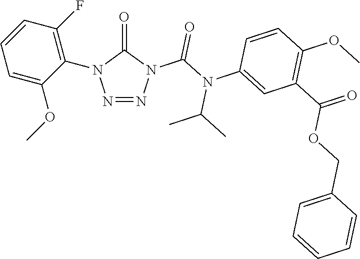 [SMILES: COc1ccc(N(C(=O)n2nnn(-c3c(F)cccc3OC)c2=O)C(C)C)cc1C(=O)OCc1ccccc1] |
| 2153 | 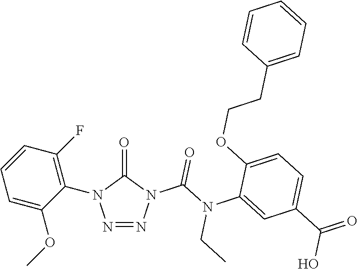 [SMILES: CCN(C(=O)n1nnn(-c2c(F)cccc2OC)c1=O)c1cc(C(=O)O)ccc1OCCc1ccccc1] |
| 2154 | 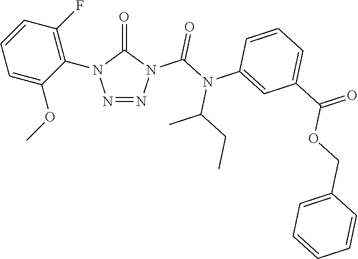 [SMILES: CCC(C)N(C(=O)n1nnn(-c2c(F)cccc2OC)c1=O)c1cccc(C(=O)OCc2ccccc2)c1] |
| 2155 | 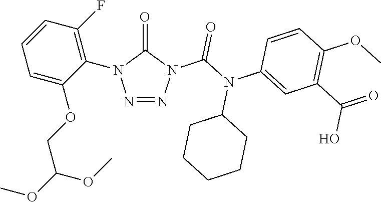 |
| 2156 | 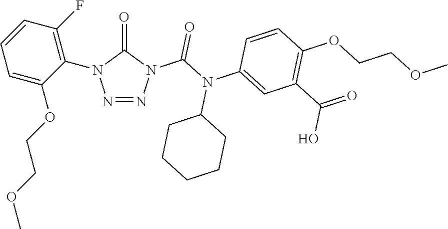 [SMILES: COCCOc1ccc(N(C(=O)n2nnn(-c3c(F)cccc3OCCOC)c2=O)C2CCCCC2)cc1C(=O)O] |
| 2157 | 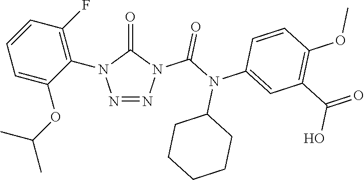 [SMILES: COc1ccc(N(C(=O)n2nnn(-c3c(F)cccc3OC(C)C)c2=O)C2CCCCC2)cc1C(=O)O] |
| 2158 | 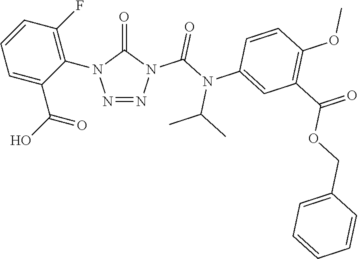 [SMILES: COc1ccc(N(C(=O)n2nnn(-c3c(F)cccc3C(=O)O)c2=O)C(C)C)cc1C(=O)OCc1ccccc1] |
| 2159 | 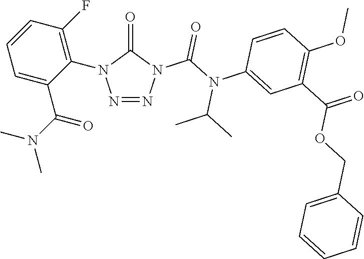 [SMILES: COc1ccc(N(C(=O)n2nnn(-c3c(F)cccc3C(=O)N(C)C)c2=O)C(C)C)cc1C(=O)OCc1ccccc1] |
| 2160 | 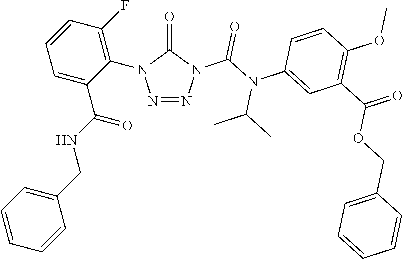 [SMILES: COc1ccc(N(C(=O)n2nnn(-c3c(F)cccc3C(=O)NCc3ccccc3)c2=O)C(C)C)cc1C(=O)OCc1ccccc1] |
| 2161 | 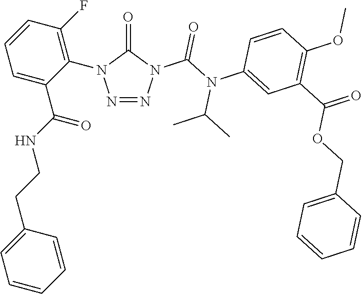 [SMILES: COc1ccc(N(C(=O)n2nnn(-c3c(F)cccc3C(=O)NCCc3ccccc3)c2=O)C(C)C)cc1C(=O)OCc1ccccc1] |
| 2162 | 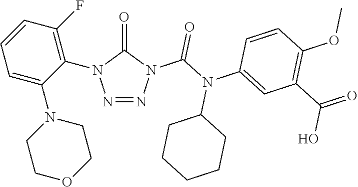 [SMILES: COc1ccc(N(C(=O)n2nnn(-c3c(F)cccc3N3CCOCC3)c2=O)C2CCCCC2)cc1C(=O)O] |
| 2163 | 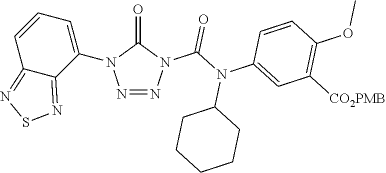 |
| 2164 | 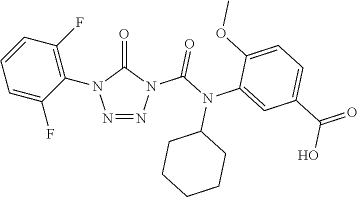 [SMILES: COc1ccc(C(=O)O)cc1N(C(=O)n1nnn(-c2c(F)cccc2F)c1=O)C1CCCCC1] |
| 2165 | 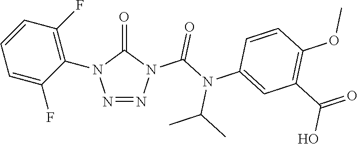 [SMILES: COc1ccc(N(C(=O)n2nnn(-c3c(F)cccc3F)c2=O)C(C)C)cc1C(=O)O] |
| 2166 | 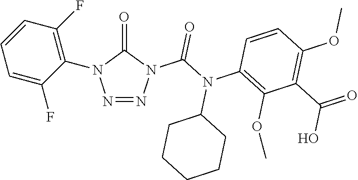 [SMILES: COc1ccc(N(C(=O)n2nnn(-c3c(F)cccc3F)c2=O)C2CCCCC2)c(OC)c1C(=O)O] |
| 2167 | 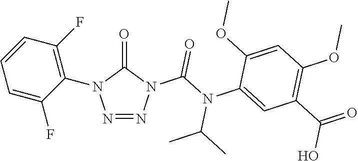 [SMILES: COc1cc(OC)c(N(C(=O)n2nnn(-c3c(F)cccc3F)c2=O)C(C)C)cc1C(=O)O] |
| 2168 | 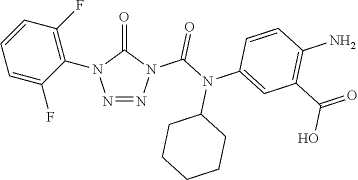 [SMILES: Nc1ccc(N(C(=O)n2nnn(-c3c(F)cccc3F)c2=O)C2CCCCC2)cc1C(=O)O] |
| 2169 | 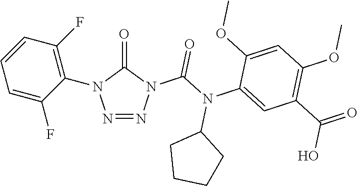 [SMILES: COc1cc(OC)c(N(C(=O)n2nnn(-c3c(F)cccc3F)c2=O)C2CCCC2)cc1C(=O)O] |
| 2170 | 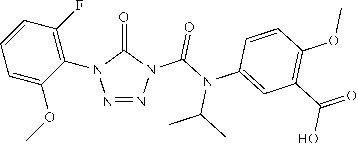 [SMILES: COc1ccc(N(C(=O)n2nnn(-c3c(F)cccc3OC)c2=O)C(C)C)cc1C(=O)O] |
| 2171 | 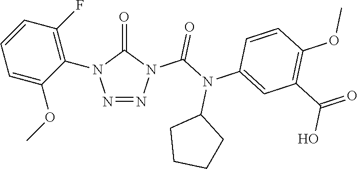 [SMILES: COc1ccc(N(C(=O)n2nnn(-c3c(F)cccc3OC)c2=O)C2CCCC2)cc1C(=O)O] |
| 2172 | 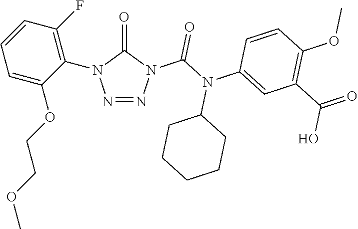 [SMILES: COCCOc1cccc(F)c1-n1nnn(C(=O)N(c2ccc(OC)c(C(=O)O)c2)C2CCCCC2)c1=O] |
| 2173 | 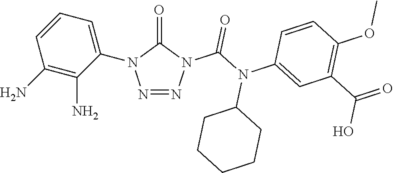 [SMILES: COc1ccc(N(C(=O)n2nnn(-c3cccc(N)c3N)c2=O)C2CCCCC2)cc1C(=O)O] |
| 2174 | 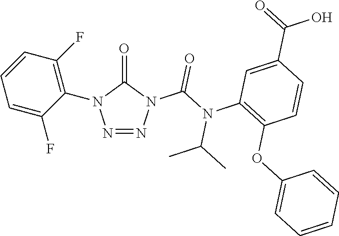 [SMILES: CC(C)N(C(=O)n1nnn(-c2c(F)cccc2F)c1=O)c1cc(C(=O)O)ccc1Oc1ccccc1] |
| 2175 | 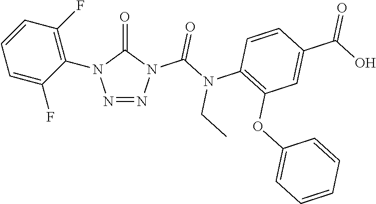 [SMILES: CCN(C(=O)n1nnn(-c2c(F)cccc2F)c1=O)c1ccc(C(=O)O)cc1Oc1ccccc1] |
| 2176 | 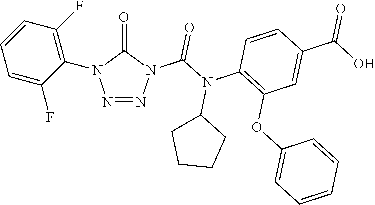 [SMILES: O=C(O)c1ccc(N(C(=O)n2nnn(-c3c(F)cccc3F)c2=O)C2CCCC2)c(Oc2ccccc2)c1] |
| 2177 | 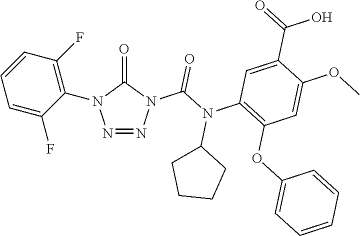 [SMILES: COc1cc(Oc2ccccc2)c(N(C(=O)n2nnn(-c3c(F)cccc3F)c2=O)C2CCCC2)cc1C(=O)O] |
| 2178 | 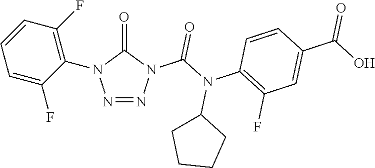 [SMILES: O=C(O)c1ccc(N(C(=O)n2nnn(-c3c(F)cccc3F)c2=O)C2CCCC2)c(F)c1] |
| 2179 | 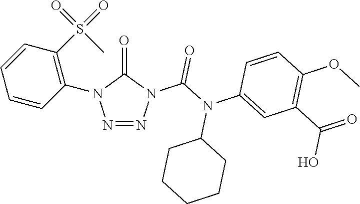 [SMILES: COc1ccc(N(C(=O)n2nnn(-c3ccccc3S(C)(=O)=O)c2=O)C2CCCCC2)cc1C(=O)O] |
| 2180 | 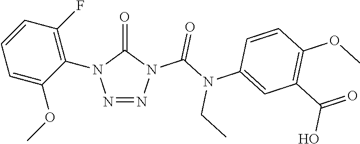 [SMILES: CCN(C(=O)n1nnn(-c2c(F)cccc2OC)c1=O)c1ccc(OC)c(C(=O)O)c1] |
| 2181 | 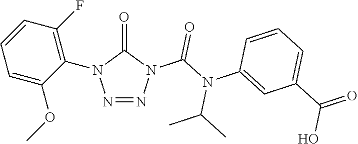 [SMILES: COc1cccc(F)c1-n1nnn(C(=O)N(c2cccc(C(=O)O)c2)C(C)C)c1=O] |
| 2182 | 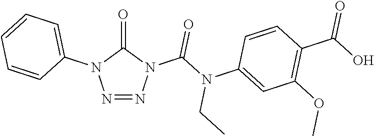 [SMILES: CCN(C(=O)n1nnn(-c2ccccc2)c1=O)c1ccc(C(=O)O)c(OC)c1] |
| 2183 | 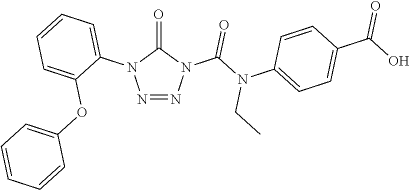 [SMILES: CCN(C(=O)n1nnn(-c2ccccc2Oc2ccccc2)c1=O)c1ccc(C(=O)O)cc1] |
| 2184 | 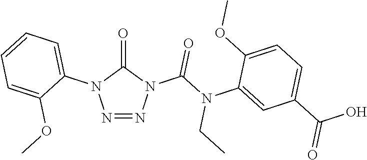 [SMILES: CCN(C(=O)n1nnn(-c2ccccc2OC)c1=O)c1cc(C(=O)O)ccc1OC] |
| 2185 | 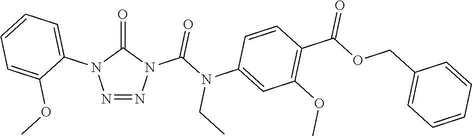 [SMILES: CCN(C(=O)n1nnn(-c2ccccc2OC)c1=O)c1ccc(C(=O)OCc2ccccc2)c(OC)c1] |
| 2186 | 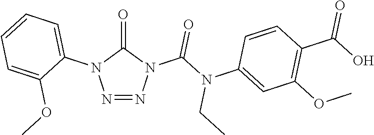 [SMILES: CCN(C(=O)n1nnn(-c2ccccc2OC)c1=O)c1ccc(C(=O)O)c(OC)c1] |
| 2187 | 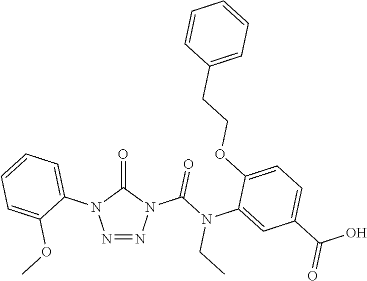 [SMILES: CCN(C(=O)n1nnn(-c2ccccc2OC)c1=O)c1cc(C(=O)O)ccc1OCCc1ccccc1] |
| 2188 | 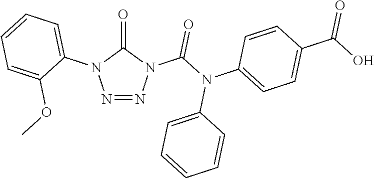 [SMILES: COc1ccccc1-n1nnn(C(=O)N(c2ccccc2)c2ccc(C(=O)O)cc2)c1=O] |
| 2189 | 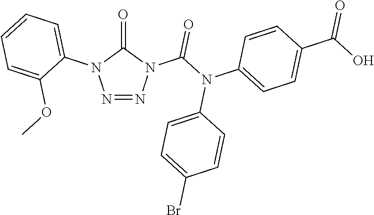 [SMILES: COc1ccccc1-n1nnn(C(=O)N(c2ccc(Br)cc2)c2ccc(C(=O)O)cc2)c1=O] |
| 2190 | 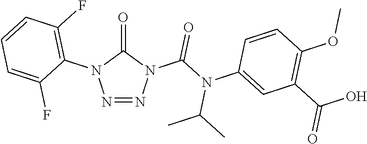 [SMILES: COc1ccc(N(C(=O)n2nnn(-c3c(F)cccc3F)c2=O)C(C)C)cc1C(=O)O] |
| 2191 | 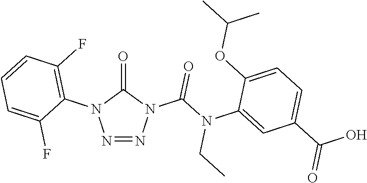 [SMILES: CCN(C(=O)n1nnn(-c2c(F)cccc2F)c1=O)c1cc(C(=O)O)ccc1OC(C)C] |
| 2192 | 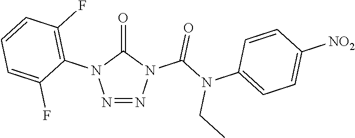 [SMILES: CCN(C(=O)n1nnn(-c2c(F)cccc2F)c1=O)c1ccc([N+](=O)[O-])cc1] |
| 2193 | 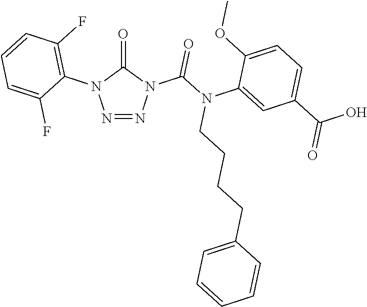 [SMILES: COc1ccc(C(=O)O)cc1N(CCCCc1ccccc1)C(=O)n1nnn(-c2c(F)cccc2F)c1=O] |
| 2194 | 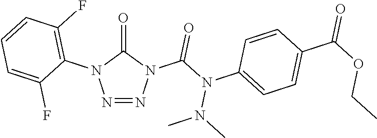 [SMILES: CCOC(=O)c1ccc(N(C(=O)n2nnn(-c3c(F)cccc3F)c2=O)N(C)C)cc1] |
| 2195 | 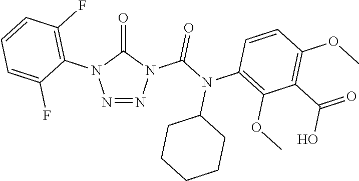 [SMILES: COc1ccc(N(C(=O)n2nnn(-c3c(F)cccc3F)c2=O)C2CCCCC2)c(OC)c1C(=O)O] |
| 2196 | 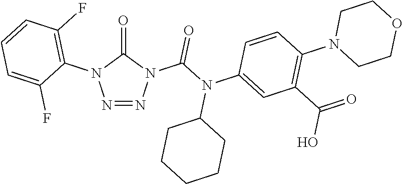 [SMILES: O=C(O)c1cc(N(C(=O)n2nnn(-c3c(F)cccc3F)c2=O)C2CCCCC2)ccc1N1CCOCC1] |
| 2197 | 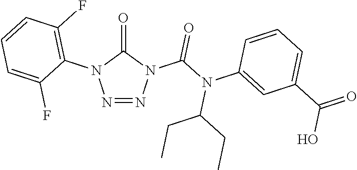 [SMILES: CCC(CC)N(C(=O)n1nnn(-c2c(F)cccc2F)c1=O)c1cccc(C(=O)O)c1] |
| 2198 | 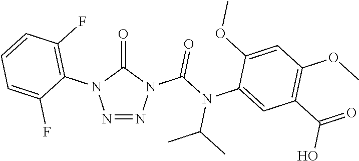 [SMILES: COc1cc(OC)c(N(C(=O)n2nnn(-c3c(F)cccc3F)c2=O)C(C)C)cc1C(=O)O] |
| 2199 | 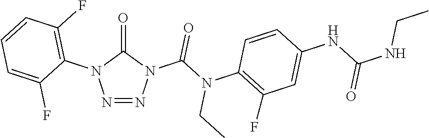 [SMILES: CCNC(=O)Nc1ccc(N(CC)C(=O)n2nnn(-c3c(F)cccc3F)c2=O)c(F)c1] |
| 2200 | 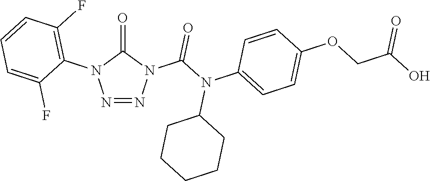 [SMILES: O=C(O)COc1ccc(N(C(=O)n2nnn(-c3c(F)cccc3F)c2=O)C2CCCCC2)cc1] |
| 2201 | 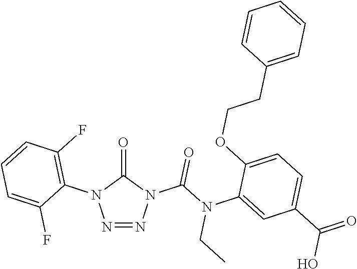 [SMILES: CCN(C(=O)n1nnn(-c2c(F)cccc2F)c1=O)c1cc(C(=O)O)ccc1OCCc1ccccc1] |
| 2202 | 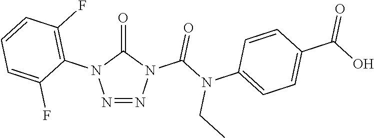 [SMILES: CCN(C(=O)n1nnn(-c2c(F)cccc2F)c1=O)c1ccc(C(=O)O)cc1] |
| 2203 | 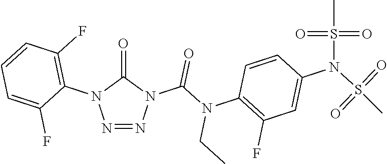 [SMILES: CCN(C(=O)n1nnn(-c2c(F)cccc2F)c1=O)c1ccc(N(S(C)(=O)=O)S(C)(=O)=O)cc1F] |
| 2204 | 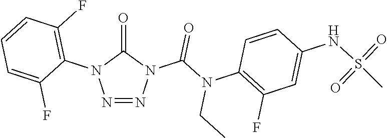 [SMILES: CCN(C(=O)n1nnn(-c2c(F)cccc2F)c1=O)c1ccc(NS(C)(=O)=O)cc1F] |
| 2205 | 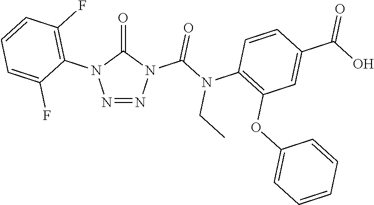 [SMILES: CCN(C(=O)n1nnn(-c2c(F)cccc2F)c1=O)c1ccc(C(=O)O)cc1Oc1ccccc1] |
| 2206 | 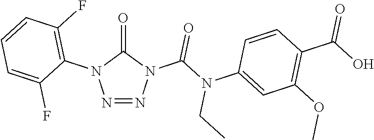 [SMILES: CCN(C(=O)n1nnn(-c2c(F)cccc2F)c1=O)c1ccc(C(=O)O)c(OC)c1] |
| 2207 | 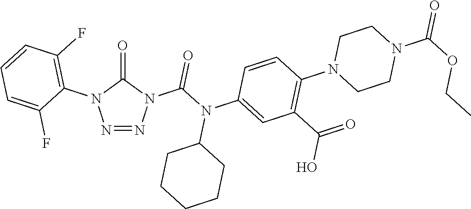 [SMILES: CCOC(=O)N1CCN(c2ccc(N(C(=O)n3nnn(-c4c(F)cccc4F)c3=O)C3CCCCC3)cc2C(=O)O)CC1] |
| 2208 | 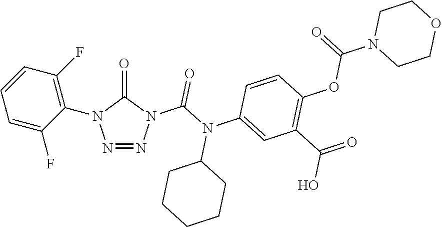 [SMILES: O=C(O)c1cc(N(C(=O)n2nnn(-c3c(F)cccc3F)c2=O)C2CCCCC2)ccc1OC(=O)N1CCOCC1] |
| 2209 | 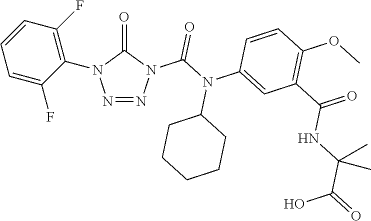 [SMILES: COc1ccc(N(C(=O)n2nnn(-c3c(F)cccc3F)c2=O)C2CCCCC2)cc1C(=O)NC(C)(C)C(=O)O] |
| 2210 | 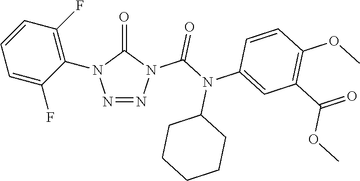 [SMILES: COC(=O)c1cc(N(C(=O)n2nnn(-c3c(F)cccc3F)c2=O)C2CCCCC2)ccc1OC] |
| 2211 | 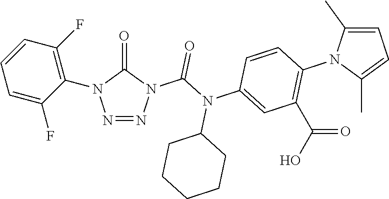 [SMILES: Cc1ccc(C)n1-c1ccc(N(C(=O)n2nnn(-c3c(F)cccc3F)c2=O)C2CCCCC2)cc1C(=O)O] |
| 2212 | 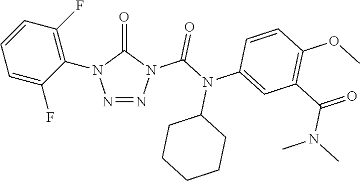 [SMILES: COc1ccc(N(C(=O)n2nnn(-c3c(F)cccc3F)c2=O)C2CCCCC2)cc1C(=O)N(C)C] |
| 2213 | 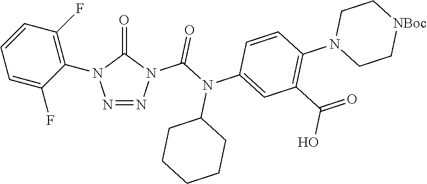 [SMILES: CC(C)(C)OC(=O)N1CCN(c2ccc(N(C(=O)n3nnn(-c4c(F)cccc4F)c3=O)C3CCCCC3)cc2C(=O)O)CC1] |
| 2214 | 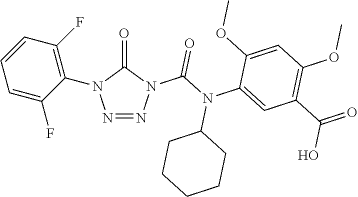 [SMILES: COc1cc(OC)c(N(C(=O)n2nnn(-c3c(F)cccc3F)c2=O)C2CCCCC2)cc1C(=O)O] |
| 2215 | 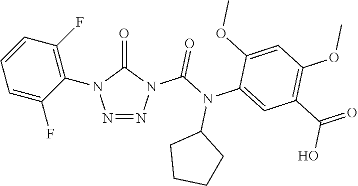 [SMILES: COc1cc(OC)c(N(C(=O)n2nnn(-c3c(F)cccc3F)c2=O)C2CCCC2)cc1C(=O)O] |
| 2216 | 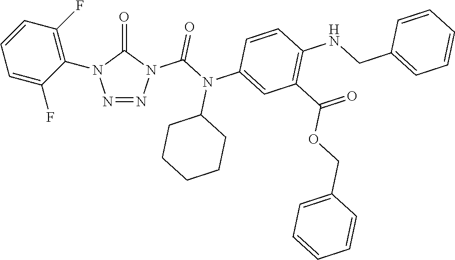 [SMILES: O=C(OCc1ccccc1)c1cc(N(C(=O)n2nnn(-c3c(F)cccc3F)c2=O)C2CCCCC2)ccc1NCc1ccccc1] |
| 2217 | 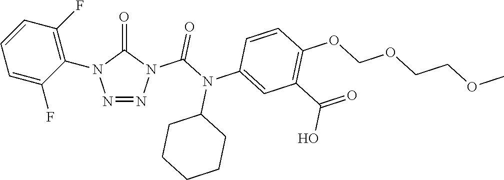 [SMILES: COCCOCOc1ccc(N(C(=O)n2nnn(-c3c(F)cccc3F)c2=O)C2CCCCC2)cc1C(=O)O] |
| 2218 | 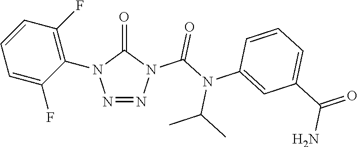 [SMILES: CC(C)N(C(=O)n1nnn(-c2c(F)cccc2F)c1=O)c1cccc(C(N)=O)c1] |
| 2219 | 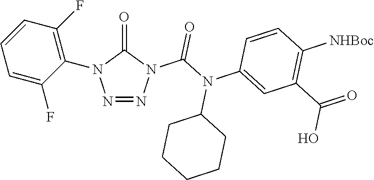 [SMILES: CC(C)(C)OC(=O)Nc1ccc(N(C(=O)n2nnn(-c3c(F)cccc3F)c2=O)C2CCCCC2)cc1C(=O)O] |
| 2220 | 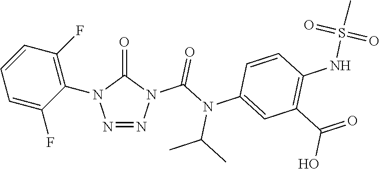 [SMILES: CC(C)N(C(=O)n1nnn(-c2c(F)cccc2F)c1=O)c1ccc(NS(C)(=O)=O)c(C(=O)O)c1] |
| 2221 | 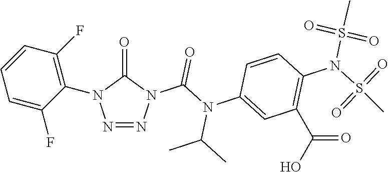 [SMILES: CC(C)N(C(=O)n1nnn(-c2c(F)cccc2F)c1=O)c1ccc(N(S(C)(=O)=O)S(C)(=O)=O)c(C(=O)O)c1] |
| 2222 | 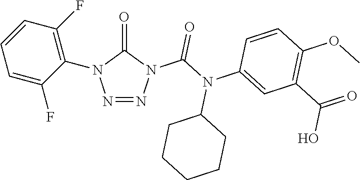 [SMILES: COc1ccc(N(C(=O)n2nnn(-c3c(F)cccc3F)c2=O)C2CCCCC2)cc1C(=O)O] |
| 2223 | 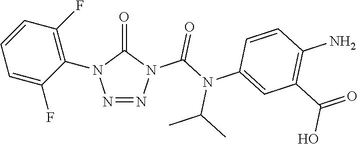 [SMILES: CC(C)N(C(=O)n1nnn(-c2c(F)cccc2F)c1=O)c1ccc(N)c(C(=O)O)c1] |
| 2224 | 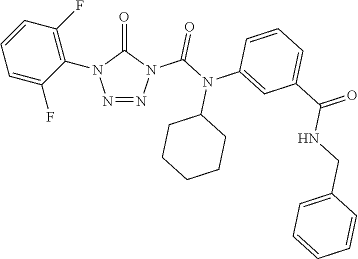 [SMILES: O=C(NCc1ccccc1)c1cccc(N(C(=O)n2nnn(-c3c(F)cccc3F)c2=O)C2CCCCC2)c1] |
| 2225 | 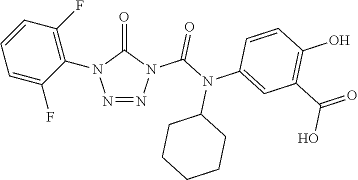 [SMILES: O=C(O)c1cc(N(C(=O)n2nnn(-c3c(F)cccc3F)c2=O)C2CCCCC2)ccc1O] |
| 2226 | 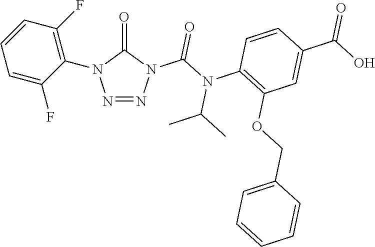 [SMILES: CC(C)N(C(=O)n1nnn(-c2c(F)cccc2F)c1=O)c1ccc(C(=O)O)cc1OCc1ccccc1] |
| 2227 | 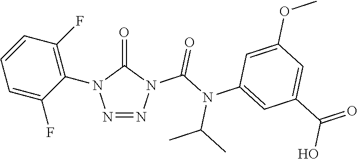 [SMILES: COc1cc(C(=O)O)cc(N(C(=O)n2nnn(-c3c(F)cccc3F)c2=O)C(C)C)c1] |
| 2228 | 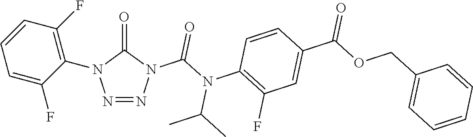 [SMILES: CC(C)N(C(=O)n1nnn(-c2c(F)cccc2F)c1=O)c1ccc(C(=O)OCc2ccccc2)cc1F] |
| 2229 | 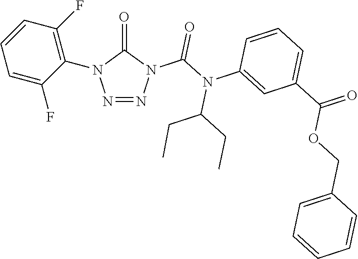 [SMILES: CCC(CC)N(C(=O)n1nnn(-c2c(F)cccc2F)c1=O)c1cccc(C(=O)OCc2ccccc2)c1] |
| 2230 | 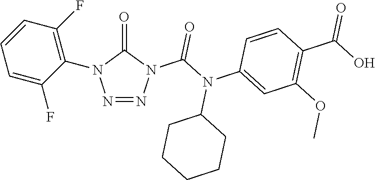 [SMILES: COc1cc(N(C(=O)n2nnn(-c3c(F)cccc3F)c2=O)C2CCCCC2)ccc1C(=O)O] |
| 2231 | 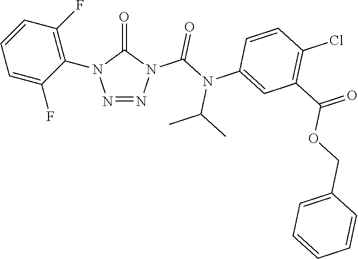 [SMILES: CC(C)N(C(=O)n1nnn(-c2c(F)cccc2F)c1=O)c1ccc(Cl)c(C(=O)OCc2ccccc2)c1] |
| 2232 | 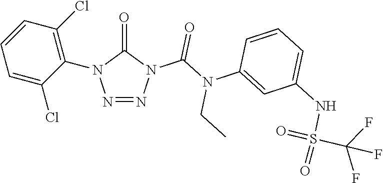 [SMILES: CCN(C(=O)n1nnn(-c2c(Cl)cccc2Cl)c1=O)c1cccc(NS(=O)(=O)C(F)(F)F)c1] |
| 2233 | 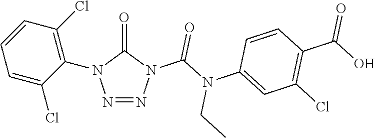 [SMILES: CCN(C(=O)n1nnn(-c2c(Cl)cccc2Cl)c1=O)c1ccc(C(=O)O)c(Cl)c1] |
| 2234 | 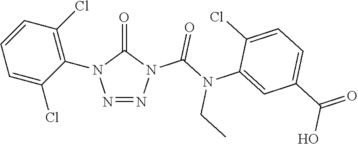 [SMILES: CCN(C(=O)n1nnn(-c2c(Cl)cccc2Cl)c1=O)c1cc(C(=O)O)ccc1Cl] |
| 2235 | 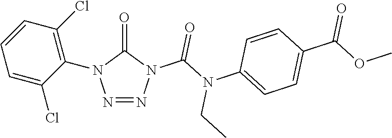 [SMILES: CCN(C(=O)n1nnn(-c2c(Cl)cccc2Cl)c1=O)c1ccc(C(=O)OC)cc1] |
| 2236 | 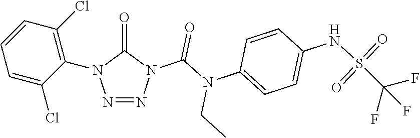 [SMILES: CCN(C(=O)n1nnn(-c2c(Cl)cccc2Cl)c1=O)c1ccc(NS(=O)(=O)C(F)(F)F)cc1] |
| 2237 | 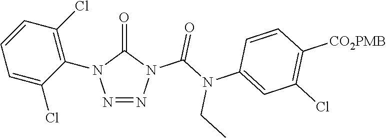 |
| 2238 | 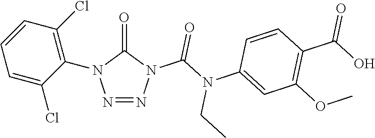 [SMILES: CCN(C(=O)n1nnn(-c2c(Cl)cccc2Cl)c1=O)c1ccc(C(=O)O)c(OC)c1] |
| 2239 | 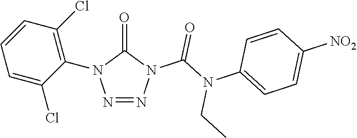 [SMILES: CCN(C(=O)n1nnn(-c2c(Cl)cccc2Cl)c1=O)c1ccc([N+](=O)[O-])cc1] |
| 2240 | 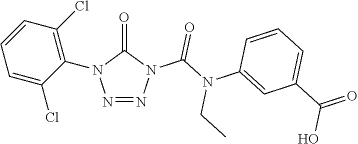 [SMILES: CCN(C(=O)n1nnn(-c2c(Cl)cccc2Cl)c1=O)c1cccc(C(=O)O)c1] |
| 2241 | 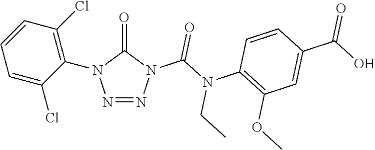 [SMILES: CCN(C(=O)n1nnn(-c2c(Cl)cccc2Cl)c1=O)c1ccc(C(=O)O)cc1OC] |
| 2242 | 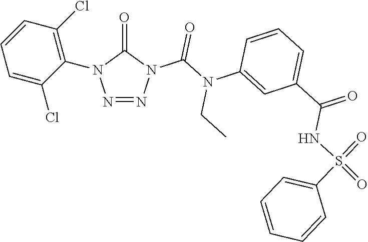 [SMILES: CCN(C(=O)n1nnn(-c2c(Cl)cccc2Cl)c1=O)c1cccc(C(=O)NS(=O)(=O)c2ccccc2)c1] |
| 2243 | 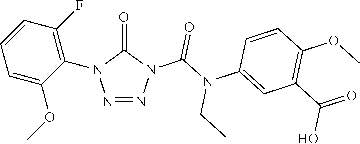 [SMILES: CCN(C(=O)n1nnn(-c2c(F)cccc2OC)c1=O)c1ccc(OC)c(C(=O)O)c1] |
| 2244 | 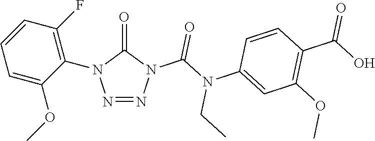 [SMILES: CCN(C(=O)n1nnn(-c2c(F)cccc2OC)c1=O)c1ccc(C(=O)O)c(OC)c1] |
| 2245 | 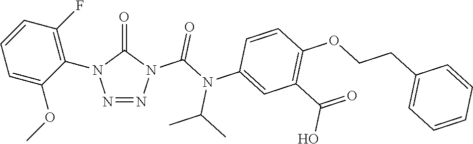 [SMILES: COc1cccc(F)c1-n1nnn(C(=O)N(c2ccc(OCCc3ccccc3)c(C(=O)O)c2)C(C)C)c1=O] |
| 2246 | 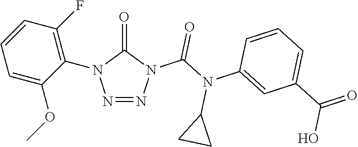 [SMILES: COc1cccc(F)c1-n1nnn(C(=O)N(c2cccc(C(=O)O)c2)C2CC2)c1=O] |
| 2247 | 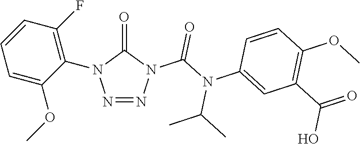 [SMILES: COc1ccc(N(C(=O)n2nnn(-c3c(F)cccc3OC)c2=O)C(C)C)cc1C(=O)O] |
| 2248 | 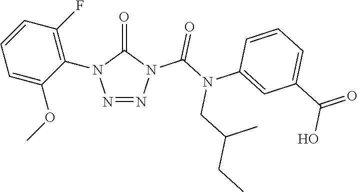 [SMILES: CCC(C)CN(C(=O)n1nnn(-c2c(F)cccc2OC)c1=O)c1cccc(C(=O)O)c1] |
| 2249 | 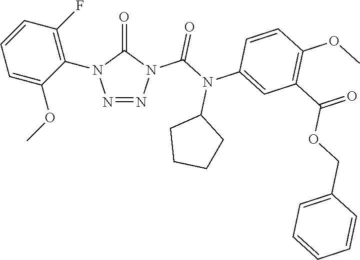 [SMILES: COc1ccc(N(C(=O)n2nnn(-c3c(F)cccc3OC)c2=O)C2CCCC2)cc1C(=O)OCc1ccccc1] |
| 2250 | 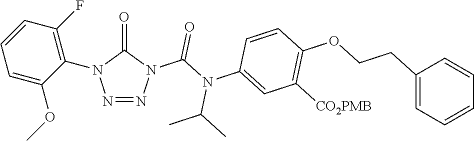 |
| 2251 | 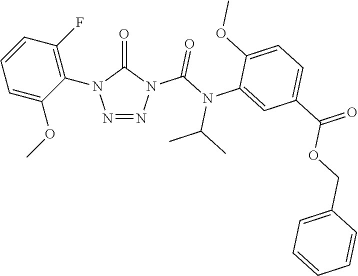 [SMILES: COc1ccc(C(=O)OCc2ccccc2)cc1N(C(=O)n1nnn(-c2c(F)cccc2OC)c1=O)C(C)C] |
| 2252 | 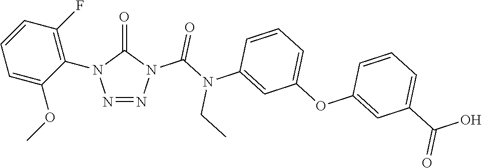 [SMILES: CCN(C(=O)n1nnn(-c2c(F)cccc2OC)c1=O)c1cccc(Oc2cccc(C(=O)O)c2)c1] |
| 2253 | 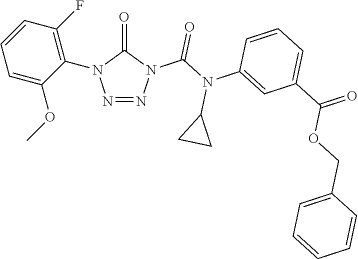 [SMILES: COc1cccc(F)c1-n1nnn(C(=O)N(c2cccc(C(=O)OCc3ccccc3)c2)C2CC2)c1=O] |
| 2254 | 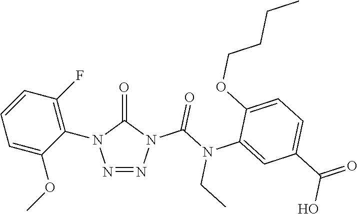 [SMILES: CCCCOc1ccc(C(=O)O)cc1N(CC)C(=O)n1nnn(-c2c(F)cccc2OC)c1=O] |
| 2255 | 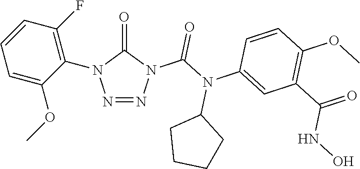 [SMILES: COc1ccc(N(C(=O)n2nnn(-c3c(F)cccc3OC)c2=O)C2CCCC2)cc1C(=O)NO] |
| 2256 | 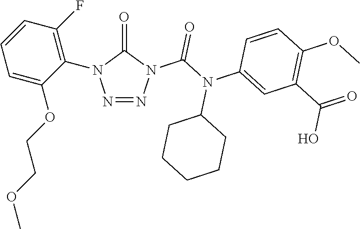 [SMILES: COCCOc1cccc(F)c1-n1nnn(C(=O)N(c2ccc(OC)c(C(=O)O)c2)C2CCCCC2)c1=O] |
| 2257 | 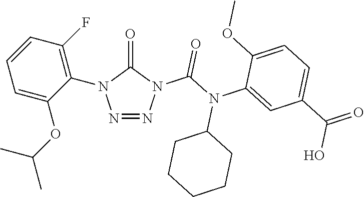 [SMILES: COc1ccc(C(=O)O)cc1N(C(=O)n1nnn(-c2c(F)cccc2OC(C)C)c1=O)C1CCCCC1] |
| 2258 | 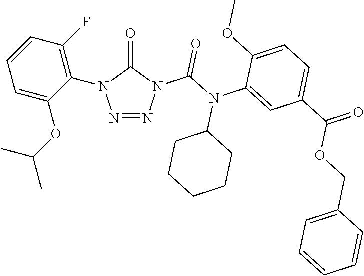 [SMILES: COc1ccc(C(=O)OCc2ccccc2)cc1N(C(=O)n1nnn(-c2c(F)cccc2OC(C)C)c1=O)C1CCCCC1] |
| 2259 | 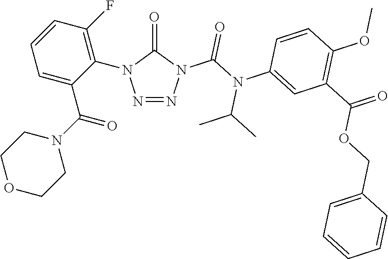 [SMILES: COc1ccc(N(C(=O)n2nnn(-c3c(F)cccc3C(=O)N3CCOCC3)c2=O)C(C)C)cc1C(=O)OCc1ccccc1] |
| 2260 | 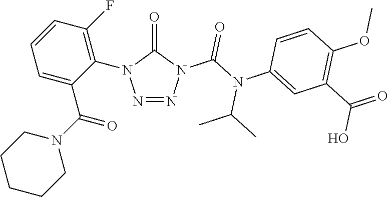 [SMILES: COc1ccc(N(C(=O)n2nnn(-c3c(F)cccc3C(=O)N3CCCCC3)c2=O)C(C)C)cc1C(=O)O] |
| 2261 | 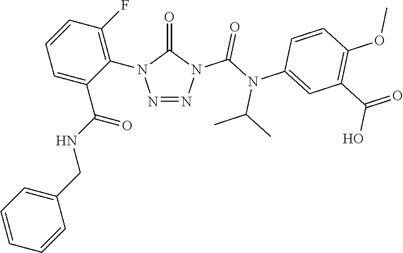 [SMILES: COc1ccc(N(C(=O)n2nnn(-c3c(F)cccc3C(=O)NCc3ccccc3)c2=O)C(C)C)cc1C(=O)O] |
| 2262 | 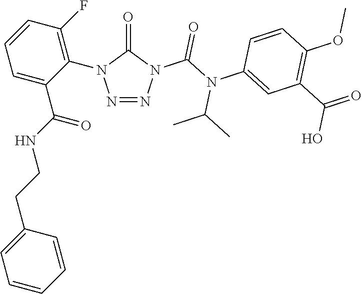 [SMILES: COc1ccc(N(C(=O)n2nnn(-c3c(F)cccc3C(=O)NCCc3ccccc3)c2=O)C(C)C)cc1C(=O)O] |
| 2263 | 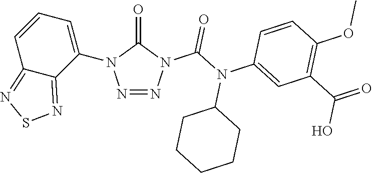 [SMILES: COc1ccc(N(C(=O)n2nnn(-c3cccc4nsnc34)c2=O)C2CCCCC2)cc1C(=O)O] |
| 2264 | 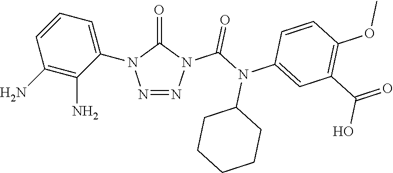 [SMILES: COc1ccc(N(C(=O)n2nnn(-c3cccc(N)c3N)c2=O)C2CCCCC2)cc1C(=O)O] |
| 2265 | 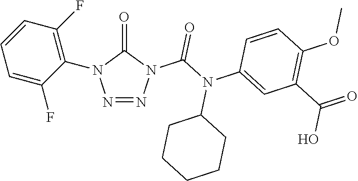 [SMILES: COc1ccc(N(C(=O)n2nnn(-c3c(F)cccc3F)c2=O)C2CCCCC2)cc1C(=O)O] |
| 2266 | 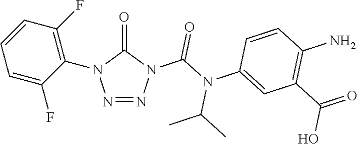 [SMILES: CC(C)N(C(=O)n1nnn(-c2c(F)cccc2F)c1=O)c1ccc(N)c(C(=O)O)c1] |
| 2267 | 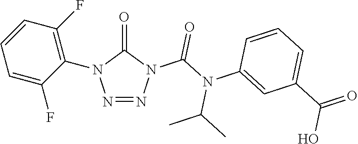 [SMILES: CC(C)N(C(=O)n1nnn(-c2c(F)cccc2F)c1=O)c1cccc(C(=O)O)c1] |
| 2268 | 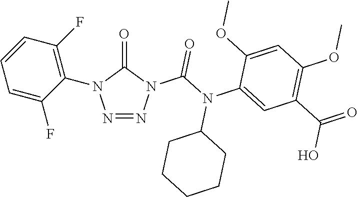 [SMILES: COc1cc(OC)c(N(C(=O)n2nnn(-c3c(F)cccc3F)c2=O)C2CCCCC2)cc1C(=O)O] |
| 2269 | 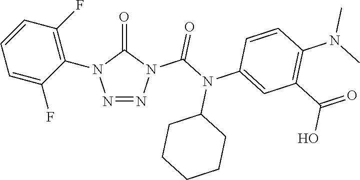 [SMILES: CN(C)c1ccc(N(C(=O)n2nnn(-c3c(F)cccc3F)c2=O)C2CCCCC2)cc1C(=O)O] |
| 2270 | 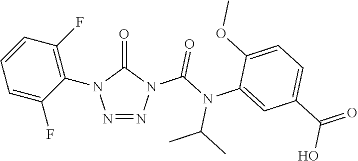 [SMILES: COc1ccc(C(=O)O)cc1N(C(=O)n1nnn(-c2c(F)cccc2F)c1=O)C(C)C] |
| 2271 | 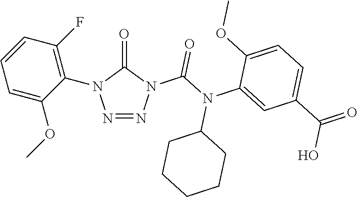 [SMILES: COc1ccc(C(=O)O)cc1N(C(=O)n1nnn(-c2c(F)cccc2OC)c1=O)C1CCCCC1] |
| 2272 | 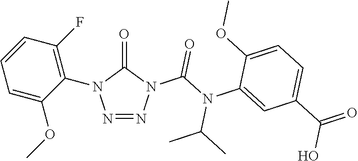 [SMILES: COc1ccc(C(=O)O)cc1N(C(=O)n1nnn(-c2c(F)cccc2OC)c1=O)C(C)C] |
| 2273 | 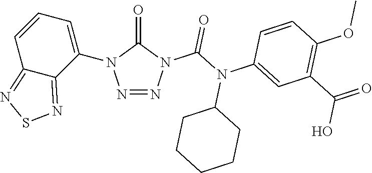 [SMILES: COc1ccc(N(C(=O)n2nnn(-c3cccc4nsnc34)c2=O)C2CCCCC2)cc1C(=O)O] |
| 2274 | 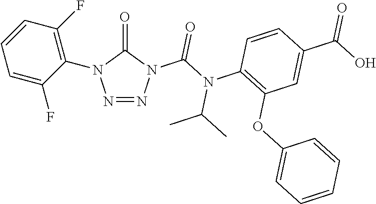 [SMILES: CC(C)N(C(=O)n1nnn(-c2c(F)cccc2F)c1=O)c1ccc(C(=O)O)cc1Oc1ccccc1] |
| 2275 | 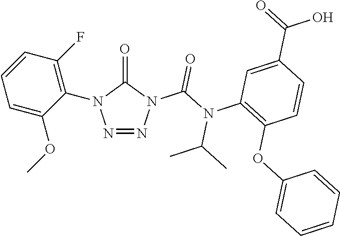 [SMILES: COc1cccc(F)c1-n1nnn(C(=O)N(c2cc(C(=O)O)ccc2Oc2ccccc2)C(C)C)c1=O] |
| 2276 | 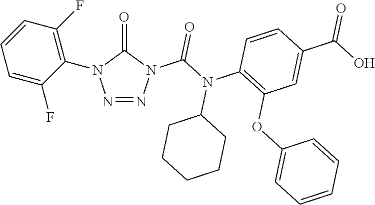 [SMILES: O=C(O)c1ccc(N(C(=O)n2nnn(-c3c(F)cccc3F)c2=O)C2CCCCC2)c(Oc2ccccc2)c1] |
| 2277 | 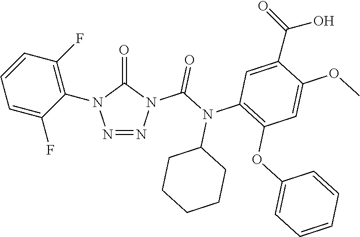 [SMILES: COc1cc(Oc2ccccc2)c(N(C(=O)n2nnn(-c3c(F)cccc3F)c2=O)C2CCCCC2)cc1C(=O)O] |
| 2278 | 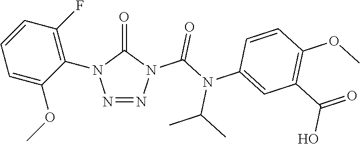 [SMILES: COc1ccc(N(C(=O)n2nnn(-c3c(F)cccc3OC)c2=O)C(C)C)cc1C(=O)O] |
| 2279 | 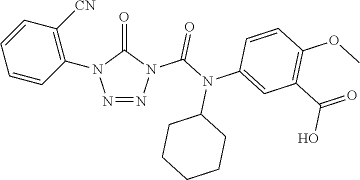 [SMILES: COc1ccc(N(C(=O)n2nnn(-c3ccccc3C#N)c2=O)C2CCCCC2)cc1C(=O)O] |
| 2280 | 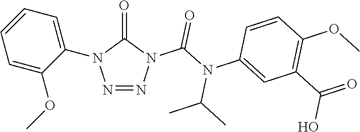 [SMILES: COc1ccc(N(C(=O)n2nnn(-c3ccccc3OC)c2=O)C(C)C)cc1C(=O)O] |
| 2281 | 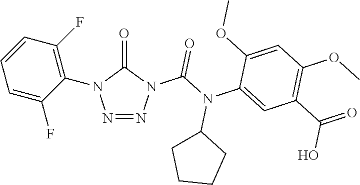 [SMILES: COc1cc(OC)c(N(C(=O)n2nnn(-c3c(F)cccc3F)c2=O)C2CCCC2)cc1C(=O)O] |
| 2282 | 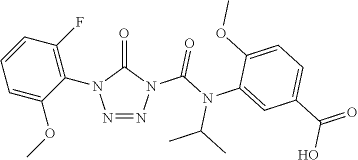 [SMILES: COc1ccc(C(=O)O)cc1N(C(=O)n1nnn(-c2c(F)cccc2OC)c1=O)C(C)C] |

In some embodiments, the FASN inhibitor is a compound disclosed in any one of International Patent Publication Nos. WO 2012/122391, WO 2014/322355, WO 2015/095767, WO 2015/105860, WO 2014/164749, WO 2013/028445, WO 2011/066211, WO 2011/056635, WO 2011/103546, WO 2014/108858, WO 2012/096928, WO 2012/037298, WO 2012/064642, WO 2013/177253, WO 2015/084606, WO 2014/039769, WO 2015/022038, WO 2014/202168, WO 2015/014446, WO 2014/044356, WO 2015/134790, WO 2011/048018, and WO 2011/140190, or U.S. Pat. Nos. 8,450,350 and 6,608,059, the compounds of each of which are herein incorporated by reference.

In some embodiments, the neurological disorder is Alzheimer's disease (AD), mild cognitive impairment (MCI), cerebral amyloid angiopathy (CAA), dementia associated with Down syndrome, or other neurodegenerative diseases characterized by the formation or accumulation of amyloid plaques including Aβ342. In some embodiments, the neurological disorder is AD, Parkinson's disease (PD), dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, Down's syndrome, multiple sclerosis, and mild cognitive impairment (MCI).

In further embodiments, the neurological disorder is a proteopathy, e.g., a synucleinopathy. In some embodiments, the synucleinopathy is Parkinson's disease (PD), dementia with Lewy bodies, pure autonomic failure, multiple system atrophy, incidental Lewy body disease, pantothenate kinase-associated neurodegeneration, Alzheimer's disease, Down's Syndrome, Gaucher disease, or the Parkinsonism-dementia complex of Guam. In some embodiments, the Parkinson's disease does not include a PINK1 mutation. In some embodiments, the Parkinson's disease is sporadic Parkinson's disease.

In further embodiments, the proteopathy is AD, Alexander disease, amyotrophic lateral sclerosis (ALS), a prion disease (e.g., Creutzfeldt-Jakob disease), Huntington's disease, Machado-Joseph disease, Pick's disease, or frontotemporal dementia.

In some embodiments, the neurological disorder is a neurodegenerative disorder, e.g., Alpers' disease, ataxia telangectsia, Canavan disease, Cockayne syndrome, corticobasal degeneration, Kennedy's disease, Krabbe disease, Pelizaeus-Merzbacher disease, primary lateral sclerosis, Refsum's disease, Sandhoff disease, Schilder's disease, Steele-Richardson-Olszewski disease, tabes dorsalis, vascular dementia, or Guillain-Barre Syndrome. In further embodiments, the neurological disorder is an ApoE-associated neurodegenerative disorder, e.g., AD, vascular cognitive impairment, cerebral amyloid angiopathy, traumatic brain injury, or multiple sclerosis. In particular embodiments, the ApoE-associated disorder is AD.

In some embodiments, the method further includes administering an additional therapeutic agent (e.g., a small molecule, an antibody or fragment thereof, or a nucleic acid) to the subject. In some embodiments, the additional therapeutic agent is a cognition-enhancing agent, an antidepressant agent, an anxiolytic agent, an antipsychotic agent, a sedative, a dopamine promoter, or an anti-tremor agent.

### Chemical Terms

It is to be understood that the terminology employed herein is for the purpose of describing particular embodiments and is not intended to be limiting.

The term “acyl,” as used herein, represents a hydrogen or an alkyl group, as defined herein, that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms). An alkylene is a divalent alkyl group.

The term “alkenyl,” as used herein, alone or in combination with other groups, refers to a straight-chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight-chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the invention can be an unsubstituted amino (i.e., —NH₂) or a substituted amino (i.e., —N(R^{N1})₂).

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₁₋₆alkyl C₆₋₁₀aryl, C₁₋₁₀alkyl C₆₋₁₀aryl, or C₁₋₂₀alkyl C₆₋₁₀aryl), such as, benzyl and phenethyl. In some embodiments, the akyl and the aryl each can be further substituted with 1, 2, 3, or 4 substituent groups as defined herein for the respective groups.

The term “azido,” as used herein, represents a —N₃ group.

The terms “C_{x-y}” and “Cₓ-C_{y},” wherein x and y are integers, are used interchangeably with one another and denote a chain of carbon atoms between x and y carbons in length.

The term “cyano,” as used herein, represents a —CN group.

The terms “carbocyclyl,” as used herein, refer to a non-aromatic C₃₋₁₂monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, norbornyl, and adamantyl.

The term “halogen,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group.

The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group.

The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to an aromatic mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁₋₆alkyl C₂₋₉heteroaryl, C₁₋₁₀alkyl C₂₋₉heteroaryl, or C₁₋₂₀alkyl C₂₋₉heteroaryl). In some embodiments, the akyl and the heteroaryl each can be further substituted with 1, 2, 3, or 4 substituent groups as defined herein for the respective groups.

The term “heterocyclyl,” as used herein, denotes a mono- or polycyclic radical having 3 to 12 atoms having at least one ring containing one, two, three, or four ring heteroatoms selected from N, O or S, wherein no ring is aromatic. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁₋₆ alkyl C₂₋₉ heterocyclyl, C₁₋₁₀ alkyl C₂₋₉ heterocyclyl, or C₁₋₂₀ alkyl C₂₋₉ heterocyclyl). In some embodiments, the akyl and the heterocyclyl each can be further substituted with 1, 2, 3, or 4 substituent groups as defined herein for the respective groups.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3^{rd} Edition (John Wiley & Sons, New York, 1999). N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, a,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2, -trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: aryl (e.g., substituted and unsubstituted phenyl), carbocyclyl (e.g., substituted and unsubstituted cycloalkyl), halogen (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbents or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99% or 99.9%) by weight relative to the other stereoisomers. When a single enantiomer is named or depicted by structure, the depicted or named enantiomer is at least 60%, 70%, 80%, 90%, 99% or 99.9% by weight optically pure. When a single diastereomer is named or depicted by structure, the depicted or named diastereomer is at least 60%, 70%, 80%, 90%, 99% or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99% or 99.9% by mole fraction pure relative to the other stereoisomers. When a single enantiomer is named or depicted by structure, the depicted or named enantiomer is at least 60%, 70%, 80%, 90%, 99% or 99.9% by mole fraction pure. When a single diastereomer is named or depicted by structure, the depicted or named diastereomer is at least 60%, 70%, 80%, 90%, 99% or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

### Definitions

The term “alpha-synuclein” refers to proteins whose amino acid sequence comprises or consists of an amino acid sequence of a naturally occurring wild-type alpha-synuclein protein as well as proteins whose amino acid sequence comprises or consists of an amino acid sequence of a naturally occurring mutant alpha-synuclein protein. Alpha-synuclein is also referred to as synuclein alpha (SNCA). Human alpha-synuclein has NCBI Gene ID NO 6622. Alpha-synuclein is considered an intrinsically disordered protein. Naturally occurring mutant alpha-synuclein proteins include A53T, A30P, E46K, H50Q, and G51D.

As used herein, “alpha-synuclein-induced toxicity” and “alpha-synuclein-mediated toxicity” are used interchangeably to refer to a reduction, impairment, or other abnormality in one or more cellular functions or structures, a reduction in growth or viability, or a combination thereof, occurring as a result of or associated with expression of an alpha-synuclein protein. In the context of a yeast cell, alpha-synuclein-mediated toxicity may be manifested as a reduction in growth or viability, e.g., reduced viability or non-viability, or a reduction, impairment, or other abnormality in one or more cellular functions or structures, e.g., reduction, impairment, or other abnormality in endocytosis or vesicle trafficking. In the context of a neuron or glial cell, e.g., a mammalian neuron or glial cell, alpha-synuclein-mediated toxicity may be manifested as a reduction in growth or viability, e.g., reduced viability or non-viability, or a reduction, impairment, or other abnormality in one or more cellular functions or structures. Cellular functions include any of the biological processes and pathways performed in a cell or by a cell, either itself or together with one or more other cells, in vitro or in vivo (e.g., in the context of a tissue or organ in vivo). In some embodiments, a cellular function is endocytosis, vesicle trafficking, axonal transport, mitochondrial function (e.g., ATP production), neurite outgrowth, neurotransmission, neurogenesis, or maintaining homeostasis. Alpha-synuclein-mediated toxicity in vivo may be manifested to a variety of extents and in a variety of ways ranging from cellular dysfunction to death. In some embodiments alpha-synuclein-mediated toxicity may be evidenced in a subject by development of a synucleinopathy or by an increased propensity to develop a synucleinopathy. In some embodiments alpha-synuclein-mediated toxicity may be manifested as a decrease or defect in cognition, behavior, or memory, as compared with a normal control. In some embodiments, contacting mammalian cells or treating a mammalian subject with an agent as described herein alleviates one or more manifestations of alpha-synuclein-mediated toxicity.

The term “apolipoprotein E (ApoE)” refers to proteins whose amino acid sequence comprises or consists of an amino acid sequence of a naturally occurring wild type ApoE protein as well as proteins whose amino acid sequence comprises or consists of an amino acid sequence of a naturally occurring allelic variant ApoE protein. Human APOE has NCBI Gene ID NO 348. APOE has three common alleles in humans: APOE ε2 (frequency ˜8%), APOE ε3 (frequency ˜80%), and APOE ε4 (frequency ˜14%). The proteins encoded by the three common APOE alleles differ at two amino acids, located at positions 112 and 158 in the mature protein. ApoE2 has cysteine at residues 112 and 158; ApoE3 has cysteine at residue 112 and arginine at residue 158; and ApoE4 has arginine at residues 112 and 158. Human ApoE protein is naturally synthesized as a precursor polypeptide of 317 amino acids, including an 18 amino acid signal sequence, which is cleaved to produce the mature 299 amino acid polypeptide. The sequence of human ApoE3 precursor polypeptide is found under NCBI RefSeq Acc. No. NP_000032.1. Naturally occurring ApoE mutations include ApoE4(L28P), which confers on carriers an increased risk for late-onset AD that remains significant even after adjusting for the effect of ApoE4 itself (Kamboh et al. Neurosci Lett. 263(2-3):129-32, 1999). Other variants include E13K, R136C, G196S, Q248E, R251G, and G278W (Tindale et al., Neurobiology of Aging 35, 727e1-727e3, 2014).

As used herein, “ApoE-induced toxicity” and “ApoE-mediated toxicity” are used interchangeably to refer to a reduction, impairment, or other abnormality in one or more cellular functions or structures, a reduction in growth or viability, or a combination thereof, occurring as a result of or associated with expression of an ApoE protein. In the context of a yeast cell, ApoE-mediated toxicity may be manifested as a reduction in growth or viability, e.g., reduced viability or non-viability, or a reduction, impairment, or other abnormality in one or more cellular functions or structures, e.g., reduction, impairment, or other abnormality in endocytosis or vesicle trafficking. In the context of a neuron or glial cell, e.g., a mammalian neuron or glial cell, ApoE-mediated toxicity may be manifested as a reduction in growth or viability, e.g., reduced viability or non-viability, or a reduction, impairment, or other abnormality in one or more cellular functions or structures. Cellular functions include any of the biological processes and pathways performed in a cell or by a cell, either itself or together with one or more other cells, in vitro or in vivo (e.g., in the context of a tissue or organ in vivo). In some embodiments, a cellular function is endocytosis, vesicle trafficking, axonal transport, mitochondrial function (e.g., ATP production), neurite outgrowth, neurotransmission, neurogenesis, or maintaining homeostasis. ApoE-mediated toxicity in vivo may be manifested to a variety of extents and in a variety of ways ranging from cellular dysfunction to death. In some embodiments ApoE-mediated toxicity may be evidenced in a subject by development of an ApoE-mediated disease (or one or more symptoms or signs of an ApoE-mediated disease) or by an increased propensity to develop an ApoE-mediated disease in subjects who express a particular ApoE isoform. In some embodiments ApoE-mediated toxicity may be manifested at least in part as an increase in the formation, deposition, accumulation, or persistence of amyloid beta aggregates or an increase in amyloid beta-mediated toxicity as compared with a normal control. In some embodiments ApoE-mediated toxicity may be manifested as a decrease or defect in cognition, behavior, or memory, as compared with a normal control. In some embodiments, contacting mammalian cells or treating a mammalian subject with an agent as described herein alleviates one or more manifestations of ApoE-mediated toxicity.

By “determining the level of a protein” is meant the detection of a protein or mRNA by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

In the practice of the methods of the present invention, an “effective amount” of any one of the compounds of the invention or a combination of any of the compounds of the invention or a pharmaceutically acceptable salt thereof, is administered via any of the usual and acceptable methods known in the art, either singly or in combination.

By “level” is meant a level of a protein or mRNA, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein or mRNA in a sample.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “neurodegenerative disorder” refers to a disorder characterized by progressive loss of the number (e.g., by cell death), structure, and/or function of neurons. In some instances, a neurodegenerative disease may be associated with protein misfolding, defects in protein degradation, genetic defects, programmed cell death, membrane damage, or other processes. Exemplary, non-limiting neurodegenerative disorders include AD, PD, ApoE-associated neurodegenerative disorders, Alpers' disease, ataxia telangectsia, Canavan disease, Cockayne syndrome, corticobasal degeneration, Kennedy's disease, Krabbe disease, Pelizaeus-Merzbacher disease, primary lateral sclerosis, Refsum's disease, Sandhoff disease, Schilder's disease, Steele-Richardson-Olszewski disease, tabes dorsalis, vascular dementia, and Guillain-Barre Syndrome.

An “ApoE-associated neurodegenerative disorder” refers to a neurodegenerative disorder that is associated with and/or mediated at least in part by an ApoE protein (e.g., ApoE4). Exemplary ApoE-associated neurodegenerative disorders include, e.g., Alzheimer's disease (AD), dementia with Lewy bodies (DLB; also referred to as “Lewy body dementia”), mild cognitive impairment (MCI), frontotemporal dementia (FTD), cerebral amyloid angiopathy (CAA), CAA-associated intracerebral hemorrhage, vascular cognitive impairment, Parkinson's disease (PD), multiple sclerosis (MS), traumatic brain injury (TBI), or Fragile X-associated tremor/ataxia syndrome.

A “neurological disorder,” as used herein, refers to a disorder of the nervous system, for example, the central nervous system (CNS). Examples of neurological disorders include, without limitation, proteopathies (e.g., synucleinopathies, tauopathies, prion diseases, and amyloidosis (e.g., A(3-amyloidosis) and/or neurodegenerative disorders (e.g., ApoE-associated neurodegenerative disorders).

It is to be understood that the above lists are not all-inclusive, and that a disorder or disease may fall within various categories. For example, Alzheimer's disease can be considered a neurodegenerative disease, a proteopathy, and, in some instances, may also be considered a synucleinopathy. Likewise, Parkinson's disease can be considered a neurodegenerative disease and a proteopathy.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of formula (I). For example pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

A “proteopathy” is a disorder that is characterized by structural abnormalities of proteins (e.g., protein misfolding and/or protein aggregation) that disrupt the function of cells, tissues, and/or organs of a subject. In some cases, misfolding can lead to loss of a protein's usual function. In other cases, a misfolded protein can gain toxic functions. In some cases, proteins can be induced to have structural abnormalities by exposure to the same (or a similar) protein that has folded into a disease-causing conformation (e.g., amyloid beta, tau, alpha-synuclein, superoxide dismutase-1 (SOD-1), polyglutamine, prion, and TAR DNA-binding protein-43 (TDP-43)). Exemplary, non-limiting proteopathies include AD, Parkinson's disease, Alexander disease, amyotrophic lateral sclerosis (ALS), a prion disease (e.g., Creutzfeldt-Jakob disease), Huntington's disease, Machado-Joseph disease, Pick's disease, or frontotemporal dementia.

By a “reference” is meant any useful reference used to compare protein or mRNA levels related to neurological disorders. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having neurological disorders; a sample from a subject that is diagnosed with cardiac artery aneurysms or stenosis; a sample from a subject that has been treated for neurological disorders; or a sample of a purified protein (e.g., any described herein) at a known normal concentration. By “reference standard or level” is meant a value or number derived from a reference sample. A “normal control value” is a predetermined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a neurological disorder. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

A “synucleinopathy” is a disorder characterized by misfolding and/or abnormal accumulation of aggregates of alpha-synuclein in the central nervous system (e.g., in neurons or glial cells). Exemplary, non-limiting synucleinopathies include Parkinson's disease (PD), dementia with Lewy bodies, pure autonomic failure, multiple system atrophy, incidental Lewy body disease, pantothenate kinase-associated neurodegeneration, Alzheimer's disease, Down's Syndrome, Gaucher disease, or the Parkinsonism-dementia complex of Guam.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

### BRIEF DESCRIPTION OF THE DRAWINGS

FIG. 1 is a schematic figure showing genes and proteins involved in lipid metabolism. ACACA produces malonyl-CoA, which is used by FASN to produce palmitate de novo. The de novo synthesized fatty acids can be elongated by elongases, such as ELOVL1, which is elongating saturated lipids, and/or desaturated by desaturases, such as SCD. The lipid metabolism is regulated by several genes, such as SREBP1 and its regulator SCAP. In addition, INSIG1 and THRSP participate in lipid metabolism regulation.

FIG. 2A and FIG. 2B are graphs showing that growth inhibition by 1,2,4-oxadiazoles occurs through same mechanism as the rescue of toxicity in the apolipoprotein E4 (ApoE4) Alzheimer's disease yeast model. (FIG. 2A) Compound 1, a representative 1,2,4-oxadiazole, was profiled in ApoE4 (top) and control (bottom) non-inducing conditions at 12-point dose (x-axis). The Y-axis shows raw OD₆₀₀. Compound 1 exhibited a bell-shaped dose-response curve (DRC) in the ApoE4 model. Rescue decreased at concentrations just above the maximal efficacy (Emax). In the control condition (bottom panel), growth decreased at this same concentration. (FIG. 2B) The relationship between Emax (rescue in ApoE4) and growth inhibition (in the control condition) correlated across 34 tested 1,2,4-oxadiazoles. The maximal rescue dose (EC100) is shown on the y-axis for ApoE4 and minimal inhibitory dose (IC100) in the control condition is shown on the x-axis. This correlation indicates that growth inhibition is caused by the same on-target activity that rescues ApoE4 toxicity.

FIG. 3A and FIG. 3B are graphs showing that exogenous oleic acid reverses growth inhibition and model rescue by Ole1/SCD-targeting 1,2,4-oxadiazoles. Growth was measured by reading OD₆₀₀ in a microplate reader and normalized to solvent control DMSO samples. (FIG. 3A) Growth inhibition (24 h) of strain GM yap1 flr1 by Ole1/SCD-targeting 1,2,4-oxadiazoles is reversed by exogenous 0.5 mM oleic/palmitoleic acid, which did not affect growth inhibition by other compounds (black dots indicate other scaffolds tested). Maximal growth inhibition across a dose range from 33 nM to 33 μM is plotted. (FIG. 3B) Rescue (40 h) of the yeast alpha-synuclein (“aSyn”) model by 1,2,4-oxadiazoles was reversed by exogenous 0.5 mM oleic/palmitoleic acid, which did not affect rescue by other scaffolds. Maximal model rescue across a dose range from 33 nM to 33 μM is plotted.

FIG. 4A and FIG. 4B are graphs showing that point mutations in yeast OLE1 confer resistance to growth inhibition and alpha-synuclein model rescue by 1,2,4-oxadiazoles. Growth was measured by reading OD₆₀₀ in a microplate reader. (FIG. 4A) Yeast cells deleted for the chromosomal copy of OLE1 and expressing OLE1 (wild-type), ole1P123T, or ole1E188Q mutants from a pRS316-based plasmid were grown in complete synthetic medium (CSM)-glucose media at the indicated doses of 1,2,4-oxadiazole Compound 2 for 24 h. Growth was normalized to samples treated with the solvent control dimethyl sulfoxide (DMSO), set as “1”. (FIG. 4B) Yeast cells deleted for the chromosomal copy of OLE1 and expressing OLE1 (Wild-type), ole1P123T, or ole1E188Q mutants from a pRS316-based plasmid were grown in CSM-galactose media (inducing expression of alpha-Synuclein) at the indicated doses of the 1,2,4-oxadiazole Compound 2 for 40 h. Growth was normalized to samples treated with the solvent control DMSO, where rescue is set as “1”.

FIG. 5 is a graph showing that a ole1Δ deletion mutant is resistant to the growth-inhibitory effects of 1,2,4-oxadiazoles, but not other compounds. Twenty-four hour growth (presented as raw OD₆₀₀) of the ole1Δ deletion strain in yeast extract-peptone-dextrose (YPD) media is shown, with drugs added at the indicated concentrations.

FIG. 6 is a graph showing that reducing OLE1 expression by deleting MGA2 rescues the growth of the ApoE4 yeast model. Yeast cells expressing ApoE4 were deleted for the MGA2 gene and their growth was assessed over time (compared to their isogenic, MGA2 wild-type counterpart). Growth was assessed by OD₆₀₀. Where indicated, 0.08 or 0.32 mM of oleic and palmitoleic acids (each) as added to the growth media in 0.01% tween (final).

FIG. 7 is a series of graphs showing that commercial Scd inhibitors target human SCD1/SCD5 in yeast. Yeast surviving solely on yeast OLE1, or human SCD1 or SCD5, were treated with four commercial Scd inhibitors at indicated concentrations. Data are expressed as a percent of the DMSO-treated condition. All four compounds potently reduced growth of both SCD1-expressing yeast and SCD5-expressing yeast, but not the strain expressing Ole1. This growth inhibition was reversed by oleic/palmitoleic acid competition, similar to the results shown in FIGS. 3A and 3B.

FIG. 8 is a series of graphs showing that 1,2,4-oxadiazoles target human SCD1 and SCD5. Three “SCD” strains expressing yeast OLE1 or human SCD1 or SCD5 were treated with five representative 1,2,4-oxadiazoles and a cycloheximide toxicity control at concentrations indicated on the log₁₀ x-axis. The y-axis indicates the percent of the DMSO-treated condition. All of the 1,2,4-oxadiazole compounds potently inhibited Ole1-expressing yeast and showed variable growth inhibition of the SCD1 or SCD5 yeast strains. These data confirm that 1,2,4-oxadiazoles target the human protein and link Scd inhibition to rescue of neurodegenerative disease models. Approximately one half of all (250) 1,2,4-oxadiazoles tested inhibited SCD1 or SCD5 in a manner that was reversed by oleic/palmitoleic acid treatment. Cyclohexamide, a translation inhibitor (top left panel), inhibited growth of all three strains with the same potency, indicating differences in growth inhibition was due to targeting the human protein.

FIG. 9A-FIG. 9D are graphs showing that treatment of yeast cells with the 1,2,4-oxadiazole Compound 2 inhibits lipid desaturation. Exponentially-growing wild-type yeast cells were treated with the indicated doses of the 1,2,4-oxadiazole Compound 2 for the indicated times before cellular lysis, lipid extraction, and analysis by global LC-MS/MS profiling. The relative abundance (fraction of total cellular lipid signal) after 1.5 h and 8 h of the most abundant saturated lipid, phosphatidylcholine 26:0, is depicted in FIGS. 9A and 9B, respectively. The relative abundance after 1.5 h and 8 h drug treatment of the most abundant lipid with 2 or more degrees of unsaturation, phosphatidylcholine 16:1; 18:1, is depicted in FIGS. 9C and 9D, respectively. The data indicate a >300-fold increase in the abundance of the saturated lipid phosphatidylcholine 26:0 after 8 h treatment with Compound 2, and a >12-fold decrease in the abundance of the unsaturated lipid phosphatidylcholine 16:1, 18:1, indicating that Compound 2 blocks cellular fatty acid desaturase activity (Ole1 is the only fatty acid desaturase in yeast).

FIG. 10 shows OLE1 mutations conferring resistance to growth inhibition to 1,2,4-oxadiazoles identified by genome sequencing of resistant mutants. Cells were plated on media containing 10 μM of the 1,2,4-oxadiazole Compound 3 and resistant colonies that emerged were isolated, and genomic DNA was prepared from mutants and the parental, drug-sensitive control strain. Genomic DNA sequence was aligned to the Saccharomyces cerevisiae reference and unique mutations in the 1,2,4-oxadiazole-resistant mutants were identified. The position of the mutations, the amino acid changes they encode, and the fold resistance (increase in minimal inhibitory concentration) of Compound 3 are shown.

FIG. 11 is a graph showing that Rab1 co-expression in U2OS cells rescues alpha-synuclein-dependent decreases in cellular ATP levels. U2OS cells were transfected with no plasmid (Mock), 2 μg of empty plasmid control (pcDNA) or 2 μg alpha-synuclein (ASYN). U2OS cells were also co-transfected with 2 μg alpha-synuclein in combination with 0.5 or 0.25 μg of mammalian Rab1a (mRab1a). ATP levels were normalized across all samples setting the Mock control as 100%. Bars depict mean values of triplicate determinations; error bars indicate standard deviation. One-way analysis of variance (ANOVA) was utilized to evaluate differences between pcDNA alone, alpha-synuclein alone, or alpha-synuclein in combination with mRab1a, with Bonferroni post-test to adjust for multiple comparisons (*** p 0.001, **** p 0.0001).

FIG. 12A and FIG. 12B are graphs showing that U2OS cells and induced pluripotent stem cell (iPSC)-derived human neurons expressed SCD1 and SCD5. (FIG. 12A) Total RNA was extracted from differentiated human neurons derived from iPSC cells obtained from a patient with alpha-synuclein gene triplication (S3), U2OS cells and rat PC-12 cells. Quantitative reverse transcription-polymerase chain reaction (RT-PCR) was performed to quantify mRNA levels of human SCD1 (hSCD1) and human SCD5 (hSCD5). All samples were normalized to hSCD1 level in U2OS cells, which was set to 1.0. Bars depict mean values of triplicate determinations; error bars indicate standard deviation. (FIG. 12B) Analysis of SCD1 protein levels in S3 neurons and U2OS cells. Protein extracts from S3 and U2OS cells were analyzed by immunoblotting with an antibody specific for human SCD1. Duplicate immunoblots were probed with an antibody against 1-tubulin as a loading control.

FIG. 13A and FIG. 13B show that knocking down SCD5 expression with siRNA rescues alpha-synuclein toxicity in U2OS cells. U2OS cells were transfected with empty vector control (“pcDNA”) or alpha-synuclein (“α-synuclein/pcDNA”) in combination with a scrambled (SCR) siRNA control (50 nM), or human SCD5 siRNA (10, 25 or 50 nM). (FIG. 13A) Cellular heath was assessed 48 h after transfection by evaluating ATP levels. Cell toxicity in the alpha-synuclein plus SCR siRNA was set as the floor of the assay, and then all samples were normalized to pcDNA with SCD5 siRNA (set to 100%) to calculate the normalized percent rescue. Bars depict mean values of triplicate determinations; error bars indicate standard deviation. A two-tailed t-test was used to compare control conditions with SCR or SCD5 siRNA (* p 0.05). Cells transfected with alpha-synuclein were analyzed together by ANOVA with Dunnett's post-test to correct for multiple comparisons (** p 0.01, **** p 0.0001). Significance is shown for the comparison of each alpha-synuclein plus SCD5 siRNA concentration compared against the alpha-synuclein plus SCR control. (FIG. 13B) Quantitative RT-PCR was utilized to confirm the levels of SCD5 mRNA. Values shown are the fold change in SCD5 mRNA levels relative to the SCR controls at 24 hours.

FIG. 14 is a graph showing that SCD inhibition with CAY10566 rescued alpha-synuclein-dependent decreases in cellular ATP levels. U2OS cells were transfected with alpha-synuclein, then treated with DMSO as a control (ASYN) or a titration of the commercially available SCD inhibitor CAY10566. Cellular ATP levels were assessed 72 h after transfection/treatment. ATP levels were normalized to the DMSO control which was set to 100%. Bars depict mean values of triplicate determinations; error bars indicate standard deviation. One-way ANOVA was utilized to evaluate CAY10566 treatment effects compared to DMSO controls, with Bonferroni post-test to adjust for multiple comparisons (* p 0.05, ** p 0.01).

FIG. 15 is a graph showing that SCD inhibition with CAY10566 reduced alpha-synuclein (A53T)-dependent neurite degeneration in transfected rat cortical neurons. Primary cultures of rat cortical neurons were co-transfected with a fluorescence reporter plasmid encoding RFP (neurite tracer) and control plasmid (empty) or plasmid containing alpha-synuclein with an A53T mutation, and treated with vehicle (DMSO) or a titration of CAY10566 ranging from 10 nM down to 10 μM as indicated. Neurite length was tracked by RFP signal every 6 h for 7 d. To follow the degeneration phase, neurite lengths were normalized to the peak neurite length for each condition and plotted over the subsequent (up to) 120 h.

FIG. 16 is a graph showing that SCD inhibition with CAY10566 reduced the cumulative risk of death in human iPSC-derived neurons harboring the alpha-synuclein A53T mutation. Human iPSC cells harboring the alpha-synuclein A53T mutation or an isogenic control line in which the mutation was corrected to wild-type were trans-differentiated into neurons. Single cells were evaluated for survival (based on overall morphology) over the course of the 192 hour study. Cell survival data was analyzed by a non-parametric Kaplan-Meier procedure to estimate survival probability, which is shown as the cumulative risk of cell death. (HR, hazard ratio; P, p value (* <0.05, ns=not significant (>0.05)); Cl, confidence interval; N, number of neurons tracked).

FIG. 17 is a graph illustrating that non-selective SCD reference inhibitor, CAY10566, reduces risk of death in A53T α-synuclein transfected human iPSC-derived neurons. Human iPSC-derived neurons were co-transfected with a fluorescence reporter plasmid encoding RFP (morphology tracer) and control plasmid (empty) or plasmid containing α-synuclein-A53T mutation (syn-A53T). Neuron groups as indicated were treated with either DMSO or CAY10566 at the indicated doses. The lifetimes of single neurons were tracked over time based on either loss of RFP fluorescence signal or morphological indicators of neuron death such as loss of neurites or cell blebbing. Kaplan-Meier survival analysis was used to generate cumulative risk of death plots. The cumulative risk of neuron death is plotted against time (hrs) for each group as indicated. CAY10566 treatment of the α-synuclein-A53T neurons was protective at each of the doses tested. Cox proportional hazard analysis was used to estimate relative risk of death, or hazard ratio (HR) and the P value was determined by the logrank test. CI, confidence interval; N, number of neurons.

FIG. 18 is a graph illustrating that an SCD5-selective inhibitor reduces risk of death in A53T α-synuclein transfected human iPSC-derived neurons. Human iPSC-derived neurons were co-transfected with a fluorescence reporter plasmid encoding RFP (morphology tracer) and control plasmid (empty) or plasmid containing α-synuclein-A53T mutation (syn-A53T). Neuron groups as indicated were treated with either DMSO or SCD5 Selective Inhibitor 1 (“SCD5-SI-1”) at the indicated doses. The lifetimes of single neurons were tracked over time based on either loss of RFP fluorescence signal or morphological indicators of neuron death such as loss of neurites or cell blebbing. Kaplan-Meier survival analysis was used to generate cumulative risk of death plots. The cumulative risk of neuron death is plotted against time (hrs) for each group as indicated. SCD5 Selective Inhibitor 1 treatment of the α-synuclein-A53T neurons was protective at each of the doses tested. Cox proportional hazard analysis was used to estimate relative risk of death, or hazard ratio (HR) and the P value was determined by the logrank test. CI, confidence interval; N, number of neurons.

FIG. 19A-FIG. 19F are a series of graphs showing an evaluation of fatty acid saturation in guinea pig brain after oral administration of SCD inhibitors. Guinea pigs were dosed orally with SCD inhibitors twice daily (every 12 hours) for 5 days. Guinea pigs were dosed with vehicle, SCD5 Selective Inhibitor 1 (“SCD5-SI-1”), SCD5 Selective Inhibitor 2 (“SCD5-SI-2”), CAY10566 (“CAY”) or SCD1/SCD5 Inhibitor 1 (“SCD1/5-1”), all compounds at 25 mg/kg with a volume-matched vehicle control. Four hours after the last dose on day 5, guinea pigs were sacrificed, and brains were removed after whole-body saline perfusion. Brains were homogenized, and fatty acids hydrolyzed from esterified lipids, which were then methylated to generate fatty acid methyl esters (FAME). Samples were evaluated on a gas chromatograph with a flame ionization detector (GC-FID) to quantify a comprehensive panel of fatty acid species. Brain samples were evaluated for levels of 16 (FIG. 19A) and 18 (FIG. 19B) carbon-containing fatty acids (C16, C18 respectively), and the desaturation index (DI) was calculated by taking the ratio of desaturated to saturated fatty acid for each species. SCD5-selective compounds SCD5-SI-1 and SCD5-SI-2, and SCD non-selective inhibitors CAY10566 and SCD1/5-1, all decreased the C16 DI, indicating they were active in modulating SCD activity in the brain and promoting a pharmacodynamic response. No significant changes were observed in the C18 DI. Brain samples were evaluated for the relative levels of the positional isomers of C16, including C16:1 n7 palmitoleic acid (FIG. 19C) or C16:1 n9 monounsaturated fatty acids (FIG. 19D). C16:1 n9 fatty acids are derived from monounsaturated C18:1 n9 fatty acids that have lost 2 carbon units due to β-oxidation. Compared to vehicle controls, all compounds decreased the levels of monounsaturated C16:1 fatty acids. FIGS. 19E and 19F show evaluation of brain samples for the relative levels of linoleic acid (18:2n6) (FIG. 19E) and gamma-linoleic acid (18:3n6) (FIG. 19F). Both species are essential omega-6 fatty acids, and both significantly increased with administration of SCD5-selective or non-selective compounds. n=8 for each group. Individual points plotted, mean indicated by black bars. Error bars represent standard deviation. Data was analyzed by one-way ANOVA with Tukey's post-hoc test to account for multiple comparisons. ** p<0.01, *** p<0.005, **** p<0.0001. Upper black bars across graph and corresponding black significance marks indicate comparison to vehicle controls. Lower bars across graph and corresponding significance marks indicate comparison between the compound-treated groups. Non-significant changes/comparisons are indicated (n.s.).

### DETAILED DESCRIPTION OF THE INVENTION

The present disclosure provides methods for the treatment of neurological disorders, e.g., by suppressing toxicity in cells related to protein misfolding and/or aggregation.

### FASN Inhibitors

FASN inhibitors include any compound described herein such as a compound of any one of Formula I-LV, or pharmaceutically acceptable salts thereof.

A number of approaches are known in the art for determining whether a compound modulates expression or activity of FASN, for example, to determine whether a compound is a FASN inhibitor, and any suitable approach can be used in the context of the invention. The FASN activity assay may be cell-based, cell-extract-based (e.g., a microsomal assay), a cell-free assay (e.g., a transcriptional assay), or make use of substantially purified proteins.

Any suitable method can be used to determine whether a compound binds to FASN, for instance, mass spectrometry, surface plasmon resonance (SPR), or immunoassays (e.g., immunoprecipitation or enzyme-linked immunosorbent assay).

Any suitable method can be used to determine whether a compound modulates expression of FASN, for instance, Northern blotting, Western blotting, RT-PCR, mass spectrometry, or RNA sequencing.

### Pharmaceutical Uses

The compounds described herein are useful in the methods of the invention and, while not bound by theory, are believed to exert their desirable effects through their ability to inhibit toxicity caused by protein misfolding and/or aggregation, e.g., α-synuclein misfolding and/or aggregation, in a cell.

Another aspect of the present invention relates to methods of treating and/or preventing a neurological disorders such as neurodegenerative diseases in a subject in need thereof. The pathology of neurodegenerative disease, may be characterized by the presence of inclusion bodies in brain tissue of affected patients.

In certain embodiments, neurological disorders that may be treated and/or prevented by the inventive methods include, but are not limited to, Alexander disease, Alpers' disease, AD, amyotrophic lateral sclerosis, ataxia telangiectasia, Canavan disease, Cockayne syndrome, corticobasal degeneration, Creutzfeldt-Jakob disease, Huntington disease, Kennedy's disease, Krabbe disease, Lewy body dementia, Machado-Joseph disease, multiple sclerosis, PD, Pelizaeus-Merzbacher disease, Pick's disease, primary lateral sclerosis, Ref sum's disease, Sandhoff disease, Schilder's disease, Steele-Richardson-Olszewski disease, tabes dorsalis, and Guillain-Barre Syndrome.

### Combination Formulations and Uses Thereof

The compounds of the invention can be combined with one or more therapeutic agents. In particular, the therapeutic agent can be one that treats or prophylactically treats any neurological disorder described herein.

Combination Therapies

A compound of the invention can be used alone or in combination with other agents that treat neurological disorders or symptoms associated therewith, or in combination with other types of treatment to treat, prevent, and/or reduce the risk of any neurological disorders. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2005). In this case, dosages of the compounds when combined should provide a therapeutic effect.

### Pharmaceutical Compositions

The compounds of the invention are preferably formulated into pharmaceutical compositions for administration to human subjects in a biologically compatible form suitable for administration in vivo. Accordingly, in another aspect, the present invention provides a pharmaceutical composition comprising a compound of the invention in admixture with a suitable diluent, carrier, or excipient.

The compounds of the invention may be used in the form of the free base, in the form of salts, solvates, and as prodrugs. All forms are within the scope of the invention. In accordance with the methods of the invention, the described compounds or salts, solvates, or prodrugs thereof may be administered to a patient in a variety of forms depending on the selected route of administration, as will be understood by those skilled in the art. The compounds of the invention may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound of the invention may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound of the invention may be incorporated with an excipient and used in the form of ingestible tablets, buccal tablets, troches, capsules, elixirs, suspensions, syrups, and wafers.

A compound of the invention may also be administered parenterally. Solutions of a compound of the invention can be prepared in water suitably mixed with a surfactant, such as hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003, 20^{th} ed.) and in The United States Pharmacopeia: The National Formulary (USP 24 NF19), published in 1999.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe.

Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter.

The compounds of the invention may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds of the invention, and/or compositions comprising a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response. In general, satisfactory results may be obtained when the compounds of the invention are administered to a human at a daily dosage of, for example, between 0.05 mg and 3000 mg (measured as the solid form). Dose ranges include, for example, between 10-1000 mg (e.g., 50-800 mg). In some embodiments, 50, 100, 150, 200, 250, 300, 350, 400, 450, 500, 550, 600, 650, 700, 750, 800, 850, 900, 950, or 1000 mg of the compound is administered. Preferred dose ranges include, for example, between 0.05-15 mg/kg or between 0.5-15 mg/kg.

Alternatively, the dosage amount can be calculated using the body weight of the patient. For example, the dose of a compound, or pharmaceutical composition thereof, administered to a patient may range from 0.1-50 mg/kg (e.g., 0.25-25 mg/kg). In exemplary, non-limiting embodiments, the dose may range from 0.5-5.0 mg/kg (e.g., 0.5, 1.0, 1.5, 2.0, 2.5, 3.0, 3.5, 4.0, 4.5, or 5.0 mg/kg) or from 5.0-20 mg/kg (e.g., 5.5, 6.0, 6.5, 7.0, 7.5, 8.0, 8.5, 9.0, 9.5, 10, 11, 12, 13, 14, 15, 16, 17, 18, 19, or 20 mg/kg).

### EXAMPLES

### Example 1. FASN Inhibition Results in a Decrease in Desaturated Fatty Acids

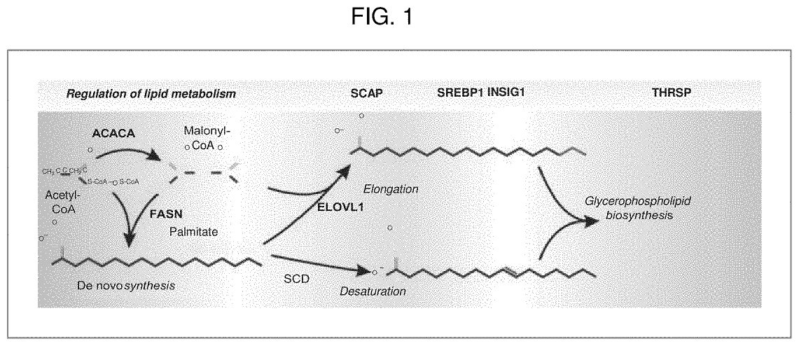

As described in Hilvo et al., Cancer Research. 71(9):3236-45, 2011, the contents of which are herein incorporated by reference, lipidomic changes to expression of lipid-related genes were observed when a comprehensive bioinformatics analysis was carried out of the mRNA expression profiles for multiple genes across data sets of 9,783 tissue samples representing 43 healthy and 68 malignant tissue types in the GeneSapiens database. On the basis of the expression and function of the genes, several genes were selected (FIG. 1) for follow-up studies by immunohistochemistry in clinical tumors and functional gene silencing studies in breast cancer cells. The selected genes function in many aspects of lipid metabolism, as illustrated in FIG. 1. In summary, ACACA, SCD (stearoyl-CoA desaturase), SREBP1, and THRSP (thyroid hormone-responsive protein) were highly expressed in clinical breast cancer samples.

### Example 2. Stearoyl-CoA Desaturase (SCD) is the Target of 1,2,4-oxadiazoles, and SCD Inhibition Results in a Decrease in Desaturated Fatty Acids and Rescues Alpha-Synuclein and ApoE4-Dependent Toxicity in Yeast Disease Models

### A. Materials and Methods

Strain Construction and OLE1 Replacement with SCD1 or SCD5

Strain GMYF was constructed from the ABC16/Green monster strain described in Suzuki et al. Nat. Methods 8(2):159-164, 2011. In this strain, YAP1 was deleted using a HIS3-MX6 cassette, and FLR1 was deleted using a NAT-MX6 cassette using standard methods. The knockout cassettes were PCR-amplified from plasmid templates (see, e.g., Bahler et al. Yeast 14(10):943-951, 1998; Longtine et al. Yeast 14(10):953-961, 1998) and transformed into yeast using lithium acetate-based transformation (Gietz et al. Methods Mol. Biol. 1205:1-12, 2014). The yap1::his3 deletion strain was selected on media lacking histidine and flr1::NAT on plates containing 100 μg/mL nourseothricin. All strains were confirmed by diagnostic PCR. Strain W303 pdr1Δ pdr3Δ was constructed from W303-1A (American Type Culture Collection (ATCC) 208352) by deleting PDR1 and PDR3 with kan-MX6 cassettes separately in MATa and MATα W303a isolates, mating, sporulating, and identifying the double deletion haploids by tetrad dissection and identification of non-parental ditype tetrads. Strain W-erg3 was derived from W303 pdr1Δ pdr3 by deleting SNQ2 with NAT-MX6, YAP1 with HIS3-MX6, and ERG3 with BleMX.

Strain ApoE-mga2Δ was generated by amplifying 1000 base pairs (bp) upstream and downstream of the MGA2 ORF in a strain in which MGA2 was deleted using a G418 (GENETICIN®) resistance cassette (kanMX) (Piotrowski et al. Proc. Natl. Acad. Sci. USA 112(12):E1490-1497, 2015) and transforming the resulting deletion cassette into the ApoE4 strain in the BY4741 (ATCC 201388) genetic background. The ApoE strain is described, for example, in International Patent Application Publication No. WO 2016/040794, which is incorporated herein by reference in its entirety.

The alpha-synuclein expression strain was made in the same manner as described in Su et al. Dis. Model Mech. 3(3-4):194-208, 2010, except that the alpha-synuclein construct lacked the green fluorescent protein (GFP) tag.

Strain ole1Δ (yeast ole1 deletion mutant) was constructed by deleting OLE1 with NAT-MX6 in BY4741, amplifying the deletion cassette from the genomic DNA of the resulting strain with primers flanking the ORF by 1000 bp upstream and downstream, transforming the resulting deletion cassette into W303 pdr1Δ pdr3Δ, and plating transformants on YPD media containing G418 (200 μg/mL) and nourseothricin (100 μg/mL) with 0.01% TWEEN®-20 and 0.5 mM oleic and palmitoleic acids.

To generate yeast strains expressing SCD1 or SCD5 as the sole desaturase, the human SCD1 and SCD5 genes were cloned from cDNAs (Harvard PlasmID database Clone ID HsCD00340237 for SCD1 and HsCD00342695 for SCD5) into yeast plasmid pRS316 (ATCC 77145) between the yeast TDH3 promoter and the CYC1 terminator. The coding sequence of yeast OLE1 was also cloned into this plasmid). These clones were then transformed into the ole1Δ strain and plated on CSM-Ura media (CSM lacking uracil) with 2% glucose (w/v) and independent colonies were isolated and amplified.

Compound Profiling Methods

All compound profiling experiments were performed using the same basic protocol. Different genetic backgrounds (e.g., gene deletions) or conditions (e.g., addition of oleic and palmitoleic acid) were replaced as indicated below.

Yeast were cultured using standard techniques in complete synthetic media (CSM) and yeast nitrogen base supplemented with 2% (w/v) carbon source (glucose, raffinose, or galactose) to regulate the expression of the toxic disease protein. An initial starter culture was inoculated in 3 mL CSM-Glucose media and incubated overnight in a 30° C. shaker incubator (225 rpm). Saturated morning cultures were then diluted 1:20 in fresh CSM-Raffinose media and grown for 6 h to an OD₆₀₀ (optical density) of 0.4-0.8 at 30° C. with shaking.

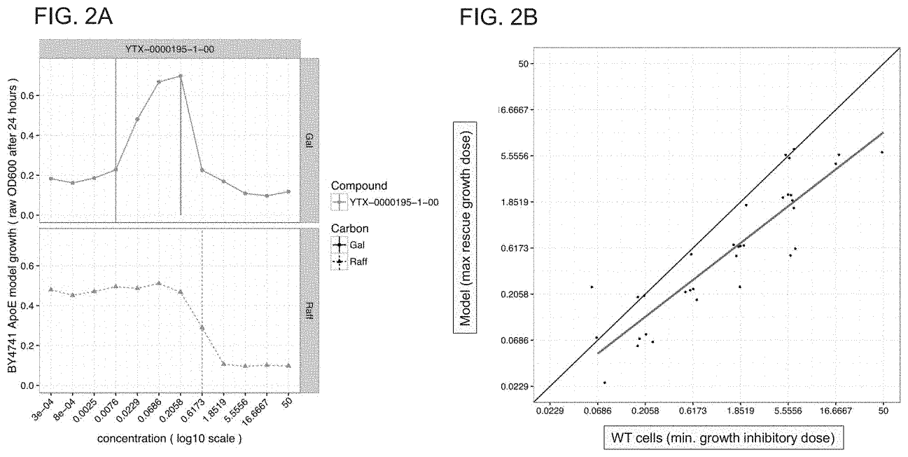

Compound stocks (10 mM in 100% DMSO) were arrayed into 384 round well, v-bottom polypropylene plates and diluted according to indicated dilution factors. Compound administration was performed in two separate steps. First, 15 μL of CSM-Galactose (induces expression of toxic protein) was dispensed into clear 384 well assay plates using a MULTIDROP™ Combi reagent dispenser. The diluted compound stock plates were then applied to the assay plates using an automated workstation (Perkin Elmer JANUS™) outfitted with a 384 pin tool containing slotted pins that deliver 100 nL of compound. The cultures described above were then diluted to a 2× concentration (0.03 and 0.08 for alpha-synuclein and ApoE, final OD₆₀₀ of 0.015 and 0.04) in CSM-Galactose. For wild-type and Ole1/SCD1/SCD5 plasmid-containing strains, the 2× cell density was 0.02. In all experiments, 15 μL culture was then dispensed into the pinned assay plate to achieve 30 μL of the 1×OD₆₀₀ culture and a top drug concentration of 33.3 M. For 96-well assays (FIGS. 2A and 2B), compound dilutions in DMSO were generated in 96 well plates and 1 μL was manually pipetted into 96 well clear bottom assay plates.

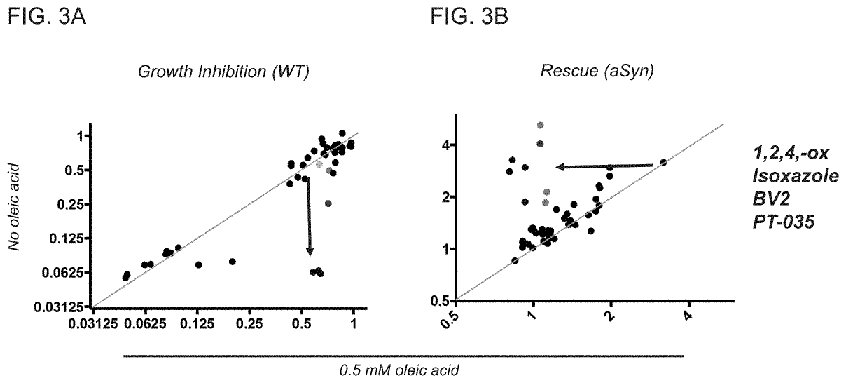

For experiments with oleic and palmitoleic acid supplementation (FIGS. 3A, 3B, 5, and 6), TWEEN®-20 was first added to culture media at a concentration of 0.01%. Oleic and palmitoleic acid were both then added at the indicated concentrations (0.08 to 0.5 mM) and mixed thoroughly prior to compound pinning or the addition of yeast.

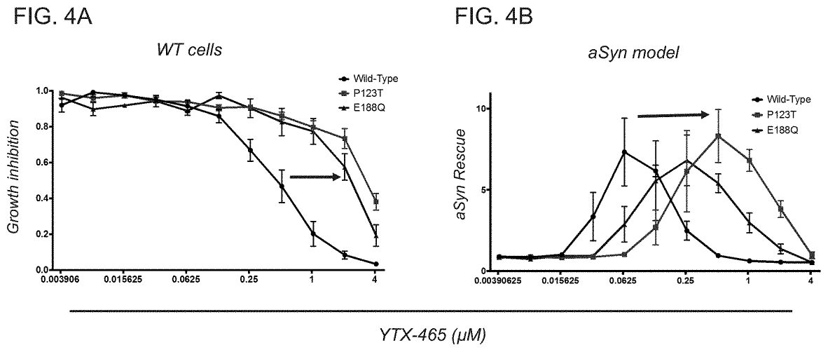

For experiments using a plasmid-borne copy of Ole1, SCD1, or SCD5 (FIGS. 4B, 7, and 8), media lacking uracil (SX-Ura, where X is glucose, raffinose, or galactose), was used for all steps of the compound profiling protocol to ensure its maintenance throughout the assay.

After yeast delivery, assay plates were incubated under humidified conditions at 30° C. for 24 to 40 h. ApoE4 rescue experiments were stopped at 24 h, aSyn experiments at 40 h, Ole1 at 24 h, and SCD1/SCD5 at 40 h. The growth of yeast was monitored by reading the OD₆₀₀ of each well using a microplate reader (Perkin Elmer EnVision™). Data were analyzed as follows. For model rescue experiments, raw data were processed by background subtracting and calculating a fold-change relative to DMSO control [(EXP−0.035)/(DMSO−0.035)—where 0.035 is the OD₆₀₀ contributed by an empty well containing 30 μL of media alone]. For growth inhibition of wild-type cells, raw data were processed by background subtracting and converting values to a percent of the nontreated condition for that strain [(EXP−0.035)/(DMSO−0.035)×100%].

Compound Sources

Compounds were sourced as follows: cycloheximide (Sigma Aldrich), A939572 (Abcam), CAY10566 (Abcam), MF-438 (Calbiochem), MK-8245 (Selleckchem), oleic acid (Sigma Aldrich), palmitoleic acid (Acros organics), mycophenolic acid (Sigma Aldrich), and tunicamycin (Cayman Chemical).

Compound 1 has the structure:

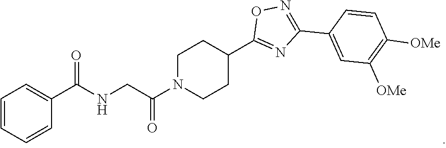 [SMILES: COc1ccc(-c2noc(C3CCN(C(=O)CNC(=O)c4ccccc4)CC3)n2)cc1OC]

Compound 1 may be synthesized by methods known in the art. For example, as shown in the scheme below:

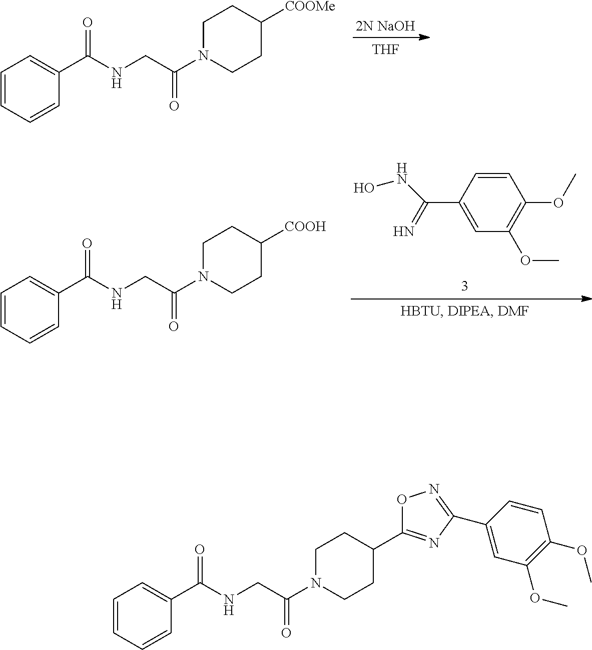

### Step 1: Preparation of 1-(2-benzamidoacetyl)piperidine-4-carboxylic Acid

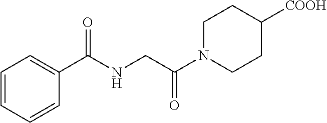 [SMILES: O=C(NCC(=O)N1CCC(C(=O)O)CC1)c1ccccc1]

To a stirred solution of methyl 1-(2-benzamidoacetyl)piperidine-4-carboxylate (5.0 g, 16.4 mmol) in tetrahydrofuran (50 mL) was added aqueous sodium hydroxide (2 M, 16.4 mL). The mixture was stirred at 20° C. for 2 h and then acidified by the addition of concentrated hydrochloric acid until pH 1. The mixture was extracted with dichloromethane (80 mL×3). The combined organic phases were washed with saturated aqueous sodium chloride solution (30 mL), dried over anhydrous sodium sulfate, filtered, and concentrated to give crude product 1-(2-benzamidoacetyl)piperidine-4-carboxylic acid (3.25 g, 11.2 mmol, 68%) as a yellow solid. ¹H NMR (400 MHz, Methanol-d4) δ 7.87 (d, J=7.5 Hz, 2H), 7.59-7.42 (m, 3H), 4.39-4.20 (m, 3H), 3.92 (d, J=14.1 Hz, 1H), 3.24 (t, J=11.5 Hz, 1H), 2.98-2.88 (m, 1H), 2.62 (s, 1H), 2.08-1.89 (m, 2H), 1.81-1.53 (m, 2H).

### Step 2: Preparation of N-(2-(4-(3-(3,4-dimethoxyphenyl)-1,2,4-oxadiazol-5-yl)piperidin-1-yl)-2-oxoethyl)benzamide

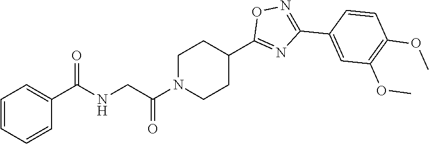 [SMILES: COc1ccc(-c2noc(C3CCN(C(=O)CNC(=O)c4ccccc4)CC3)n2)cc1OC]

To a stirred solution of 1-(2-benzamidoacetyl)piperidine-4-carboxylic acid (2.0 g, 6.89 mmol) in N,N-dimethylformamide (30 mL) was added N-hydroxy-3,4-dimethoxybenzimidamide (1.62 g, 8.27 mmol), N-ethyl-N-(propan-2-yl)propan-2-amine (2.67 g, 20.67 mmol, 3.61 mL) and 1-[bis(dimethylamino)methylene]-1H-1,2,3-triazolo[4,5-b]pyridinium 3-oxide hexafluorophosphate (2.62 g, 6.89 mmol). The mixture was stirred at 20° C. for 2 h and then warmed at 120° C. for 2 h. The reaction mixture was quenched by addition of water (40 mL), then the mixture was extracted with ethyl acetate (80 mL×3). The combined organic phases were washed with saturated aqueous sodium chloride solution (30 mL), dried over anhydrous sodium sulfate, filtered, and concentrated to give crude product that was purified by chromatography (silica, petroleum ether:ethyl acetate=20:1 to 1:2) to give a yellow solid. The yellow solid was washed with ethyl acetate (30 mL), then the mixture was filtered, and the filter cake was dried in vacuo to give N-(2-(4-(3-(3,4-dimethoxyphenyl)-1,2,4-oxadiazol-5-yl)piperidin-1-yl)-2-oxoethyl)benzamide (1.29 g, 2.86 mmol, 42%) as a white solid. ¹H NMR (400 MHz, CDCl₃) δ 7.92-7.84 (m, 2H), 7.80 (s, 1H), 7.58-7.44 (m, 3H), 7.41-7.35 (m, 1H), 7.28-7.26 (m, 2H), 6.92 (d, J=8.9 Hz, 1H), 4.58-4.47 (m, 1H), 4.32 (d, J=3.9 Hz, 2H), 3.99-3.88 (m, 7H), 3.37-3.06 (m, 3H), 2.28-2.13 (m, 2H), 2.07-1.89 (m, 2H); LCMS (ESI) [M+H]+=451.3.

Compound 2 has the structure:

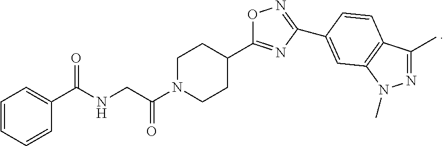 [SMILES: Cc1nn(C)c2cc(-c3noc(C4CCN(C(=O)CNC(=O)c5ccccc5)CC4)n3)ccc12]

Compound 2 may be synthesized by methods known in the art. For example, Compound 2 may be synthesized as shown in the scheme below:

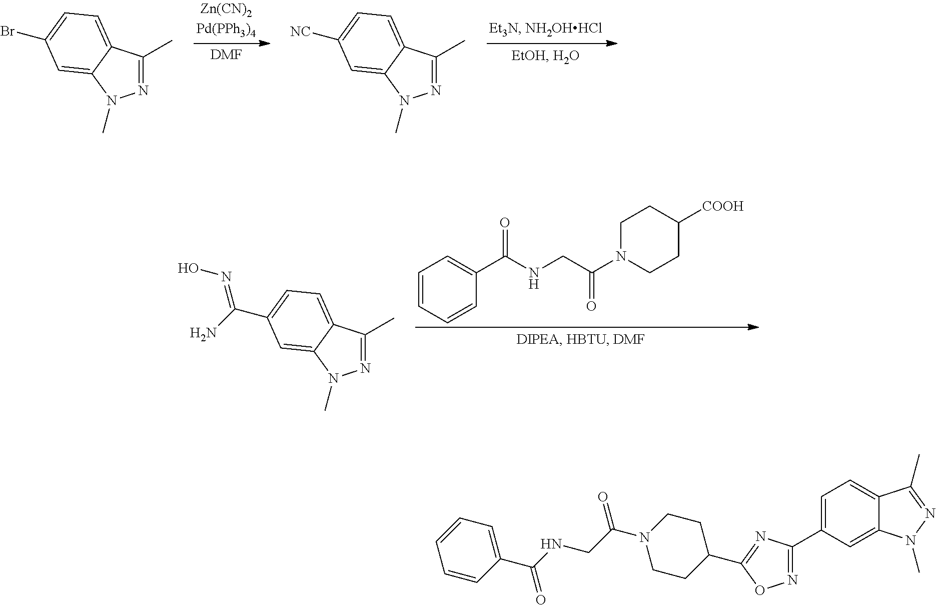

### Step 1: Preparation of 1,3-dimethyl-1H-indazole-6-carbonitrile

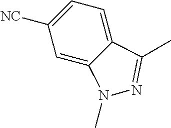

To a stirred solution of 6-bromo-1,3-dimethyl-1H-indazole (400 mg, 1.78 mmol) in N,N-dimethylformamide (5 mL) was added zinc cyanide (209 mg, 1.78 mmol, 112 μL) and tetrakis(triphenylphosphine)palladium(0) (205 mg, 178 μmol, 0.10 eq) under nitrogen. The mixture was heated at 100° C. for 16 h, then cooled to 20° C., water (10 mL) added, and the resulting mixture was extracted with ethyl acetate (40 mL×3). The combined organic phases were washed with saturated aqueous sodium chloride solution (15 mL) and dried over anhydrous sodium sulfate. The organic phase was filtered and concentrated in vacuo to give crude product. Petroleum ether (40 mL) was added to the crude product, then the mixture was filtered, and the filter cake dried in vacuo to give 1,3-dimethyl-1H-indazole-6-carbonitrile (250 mg, 1.46 mmol, 82%) as a white solid. ¹H NMR (400 MHz, CDCl₃) δ 7.78-7.71 (m, 2H), 7.34 (dd, J=1.3, 8.3 Hz, 1H), 4.07 (s, 3H), 2.61 (s, 3H).

### Step 2: Preparation of (Z)—N′-hydroxy-1,3-dimethyl-1H-indazole-6-carboximidamide

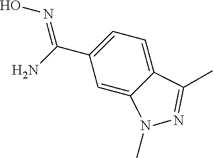

To a stirred solution of 1,3-dimethyl-1H-indazole-6-carbonitrile (100 mg, 584 μmol) in ethanol (2 mL) was added hydroxylamine hydrochloride (81 mg, 1.17 mmol), triethylamine (118 mg, 1.17 mmol, 161 μL) and water (200 μL). The mixture was heated at 75° C. for 2 h. After cooling to 20° C., water (5 mL) was added to the solution. The mixture was extracted with dichloromethane (30 mL×3). The combined organic phases were washed with saturated aqueous sodium chloride solution (5 mL) and dried over anhydrous sodium sulfate, then filtered and concentrated in vacuo to give (Z)—N′-hydroxy-1,3-dimethyl-1H-indazole-6-carboximidamide (140 mg) as a white solid. LCMS (ESI) m/z: [M+H]+=205.1.

### Step 3: Preparation of N-(2-(4-(3-(1,3-dimethyl-1H-indazol-6-yl)-1,2,4-oxadiazol-5-yl)piperidin-1-yl)-2-oxoethyl)benzamide

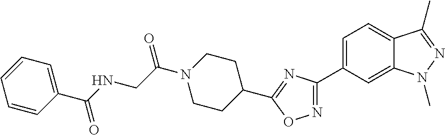 [SMILES: Cc1nn(C)c2cc(-c3noc(C4CCN(C(=O)CNC(=O)c5ccccc5)CC4)n3)ccc12]

To a stirred solution of 1-(2-benzamidoacetyl)piperidine-4-carboxylic acid (120 mg, 413 μmol) in N,N-dimethylformamide (2 mL) was added (Z)—N′-hydroxy-1,3-dimethyl-1H-indazole-6-carboximidamide (101 mg, 496 μmol), (2-(1H-benzotriazol-1-yl)-1,1,3,3-tetramethyluronium hexafluorophosphate) (156 mg, 413 μmol) and N-ethyl-N-(propan-2-yl)propan-2-amine (160 mg, 1.24 mmol, 216 μL). The mixture was stirred at 20° C. for 2 h, then heated at 120° C. for 2 h. The reaction mixture cooled then purified directly by prep-HPLC (column: Waters Xbridge 150×2.5 mm 5 μm; mobile phase: [water (10 mM ammonium carbonate)-acetonitrile]; B %: 30%-65%,12 min) to give N-(2-(4-(3-(1,3-dimethyl-1H-indazol-6-yl)-1,2,4-oxadiazol-5-yl)piperidin-1-yl)-2-oxoethyl)benzamide (46 mg, 101 μmol, 25%) as a yellow solid. ¹H NMR (400 MHz, CDCl₃) δ 8.02 (s, 1H), 7.81-7.73 (m, 3H), 7.66 (dd, J=0.6, 8.4 Hz, 1H), 7.48-7.42 (m, 1H), 7.42-7.35 (m, 2H), 7.26 (br. s., 1H), 4.46 (d, J=14.1 Hz, 1H), 4.24 (d, J=3.9 Hz, 2H), 4.01 (s, 3H), 3.86 (d, J=13.7 Hz, 1H), 3.29 (ddd, J=3.6, 10.5, 14.2 Hz, 2H), 3.13-3.04 (m, 1H), 2.53 (s, 3H), 2.26-2.15 (m, 2H), 2.04-1.89 (m, 2H); LCMS (ESI) m/z: [M+H]⁺=459.3.

Compound 3 has the structure:

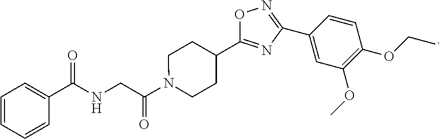 [SMILES: CCOc1ccc(-c2noc(C3CCN(C(=O)CNC(=O)c4ccccc4)CC3)n2)cc1OC]

Compound 3 may be synthesized by methods known in the art. For example, Compound 3 may be synthesized as shown in the scheme below:

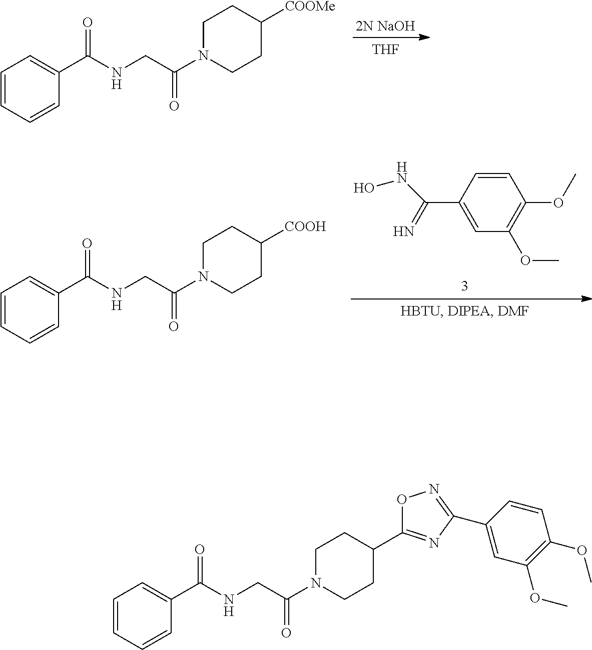

### Step 1: Preparation of 1-(2-benzamidoacetyl)piperidine-4-carboxylic Acid

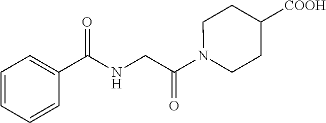 [SMILES: O=C(NCC(=O)N1CCC(C(=O)O)CC1)c1ccccc1]

To a stirred solution of methyl 1-(2-benzamidoacetyl)piperidine-4-carboxylate (5.0 g, 16.4 mmol) in tetrahydrofuran (50 mL) was added aqueous sodium hydroxide (2 M, 16.4 mL). The mixture was stirred at 20° C. for 2 h and then acidified by the addition of concentrated hydrochloric acid until pH 1. The mixture was extracted with dichloromethane (80 mL×3). The combined organic phases were washed with saturated aqueous sodium chloride solution (30 mL), dried over anhydrous sodium sulfate, filtered, and concentrated to give crude product 1-(2-benzamidoacetyl)piperidine-4-carboxylic acid (3.25 g, 11.2 mmol, 68%) as a yellow solid. ¹H NMR (400 MHz, Methanol-d4) δ 7.87 (d, J=7.5 Hz, 2H), 7.59-7.42 (m, 3H), 4.39-4.20 (m, 3H), 3.92 (d, J=14.1 Hz, 1H), 3.24 (t, J=11.5 Hz, 1H), 2.98-2.88 (m, 1H), 2.62 (s, 1H), 2.08-1.89 (m, 2H), 1.81-1.53 (m, 2H).

### Step 2: Preparation of N-(2-(4-(3-(3,4-dimethoxyphenyl)-1,2,4-oxadiazol-5-yl)piperidin-1-yl)-2-oxoethyl)benzamide

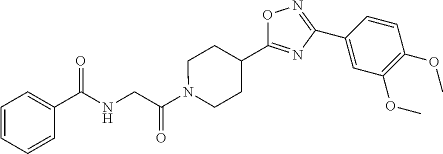 [SMILES: COc1ccc(-c2noc(C3CCN(C(=O)CNC(=O)c4ccccc4)CC3)n2)cc1OC]

To a stirred solution of 1-(2-benzamidoacetyl)piperidine-4-carboxylic acid (2.0 g, 6.89 mmol) in N,N-dimethylformamide (30 mL) was added N-hydroxy-3,4-dimethoxybenzimidamide (1.62 g, 8.27 mmol), N-ethyl-N-(propan-2-yl)propan-2-amine (2.67 g, 20.67 mmol, 3.61 mL) and 1-[bis(dimethylamino)methylene]-1H-1,2,3-triazolo[4,5-b]pyridinium 3-oxide hexafluorophosphate (2.62 g, 6.89 mmol). The mixture was stirred at 20° C. for 2 h and then warmed at 120° C. for 2 h. The reaction mixture was quenched by addition of water (40 mL), then the mixture was extracted with ethyl acetate (80 mL×3). The combined organic phases were washed with saturated aqueous sodium chloride solution (30 mL), dried over anhydrous sodium sulfate, filtered, and concentrated to give crude product that was purified by chromatography (silica, petroleum ether:ethyl acetate=20:1 to 1:2) to give a yellow solid. The yellow solid was washed with ethyl acetate (30 mL), then the mixture was filtered, and the filter cake was dried in vacuo to give N-(2-(4-(3-(3,4-dimethoxyphenyl)-1,2,4-oxadiazol-5-yl)piperidin-1-yl)-2-oxoethyl)benzamide (1.29 g, 2.86 mmol, 42%) as a white solid. ¹H NMR (400 MHz, CDCl₃) δ 7.92-7.84 (m, 2H), 7.80 (s, 1H), 7.58-7.44 (m, 3H), 7.41-7.35 (m, 1H), 7.28-7.26 (m, 2H), 6.92 (d, J=8.9 Hz, 1H), 4.58-4.47 (m, 1H), 4.32 (d, J=3.9 Hz, 2H), 3.99-3.88 (m, 7H), 3.37-3.06 (m, 3H), 2.28-2.13 (m, 2H), 2.07-1.89 (m, 2H); LCMS (ESI) [M+H]⁺=451.3.

Compound 4 has the structure:

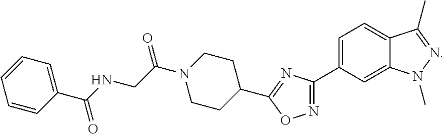 [SMILES: Cc1nn(C)c2cc(-c3noc(C4CCN(C(=O)CNC(=O)c5ccccc5)CC4)n3)ccc12]

Compound 4 may be synthesized by methods known in the art. For example, Compound 4 may be synthesized as shown in the scheme below:

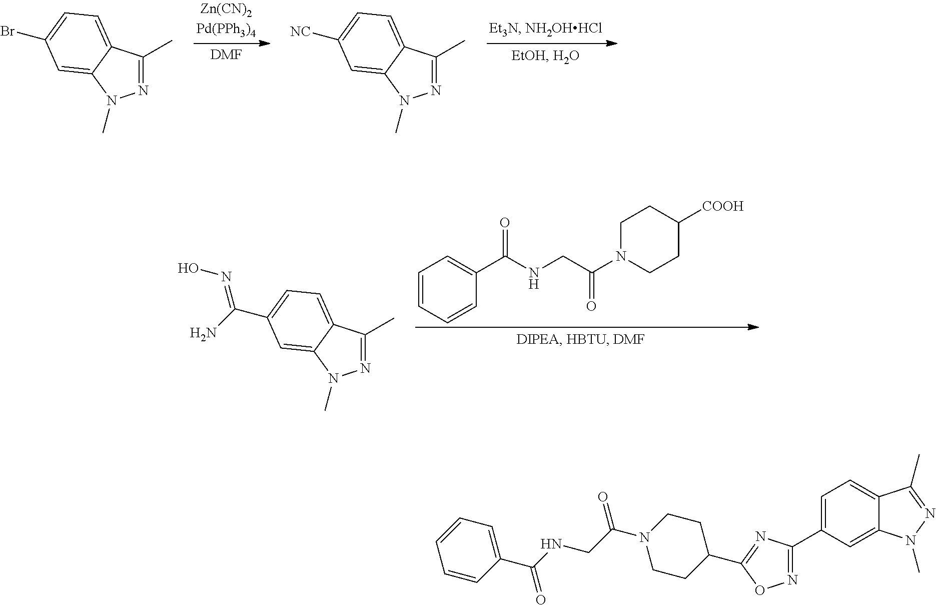

### Step 1: Preparation of 1,3-dimethyl-1H-indazole-6-carbonitrile

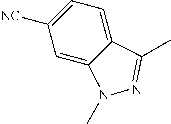

To a stirred solution of 6-bromo-1,3-dimethyl-1H-indazole (400 mg, 1.78 mmol) in N,N-dimethylformamide (5 mL) was added zinc cyanide (209 mg, 1.78 mmol, 112 μL) and tetrakis(triphenylphosphine)palladium(0) (205 mg, 178 μmol, 0.10 eq) under nitrogen. The mixture was heated at 100° C. for 16 h, then cooled to 20° C., water (10 mL) added, and the resulting mixture was extracted with ethyl acetate (40 mL×3). The combined organic phases were washed with saturated aqueous sodium chloride solution (15 mL) and dried over anhydrous sodium sulfate. The organic phase was filtered and concentrated in vacuo to give crude product. Petroleum ether (40 mL) was added to the crude product, then the mixture was filtered, and the filter cake dried in vacuo to give 1,3-dimethyl-1H-indazole-6-carbonitrile (250 mg, 1.46 mmol, 82%) as a white solid. ¹H NMR (400 MHz, CDCl₃) δ 7.78-7.71 (m, 2H), 7.34 (dd, J=1.3, 8.3 Hz, 1H), 4.07 (s, 3H), 2.61 (s, 3H).

### Step 2: Preparation of (Z)—N′-hydroxy-1,3-dimethyl-1H-indazole-6-carboximidamide

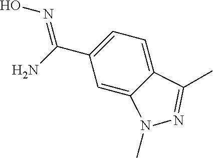

To a stirred solution of 1,3-dimethyl-1H-indazole-6-carbonitrile (100 mg, 584 μmol) in ethanol (2 mL) was added hydroxylamine hydrochloride (81 mg, 1.17 mmol), triethylamine (118 mg, 1.17 mmol, 161 μL) and water (200 μL). The mixture was heated at 75° C. for 2 h. After cooling to 20° C., water (5 mL) was added to the solution. The mixture was extracted with dichloromethane (30 mL×3). The combined organic phases were washed with saturated aqueous sodium chloride solution (5 mL) and dried over anhydrous sodium sulfate, then filtered and concentrated in vacuo to give (Z)—N′-hydroxy-1,3-dimethyl-1H-indazole-6-carboximidamide (140 mg) as a white solid. LCMS (ESI) m/z: [M+H]⁺=205.1.

### Step 3: Preparation of N-(2-(4-(3-(1,3-dimethyl-1H-indazol-6-yl)-1,2,4-oxadiazol-5-yl)piperidin-1-yl)-2-oxoethyl)benzamide

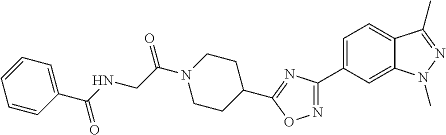 [SMILES: Cc1nn(C)c2cc(-c3noc(C4CCN(C(=O)CNC(=O)c5ccccc5)CC4)n3)ccc12]

To a stirred solution of 1-(2-benzamidoacetyl)piperidine-4-carboxylic acid (120 mg, 413 μmol) in N,N-dimethylformamide (2 mL) was added (Z)—N′-hydroxy-1,3-dimethyl-1H-indazole-6-carboximidamide (101 mg, 496 μmol), (2-(1H-benzotriazol-1-yl)-1,1,3,3-tetramethyluronium hexafluorophosphate) (156 mg, 413 μmol) and N-ethyl-N-(propan-2-yl)propan-2-amine (160 mg, 1.24 mmol, 216 μL). The mixture was stirred at 20° C. for 2 h, then heated at 120° C. for 2 h. The reaction mixture cooled then purified directly by prep-HPLC (column: Waters Xbridge 150×2.5 mm 5 μm; mobile phase: [water (10 mM ammonium carbonate)-acetonitrile]; B %: 30%-65%,12 min) to give N-(2-(4-(3-(1,3-dimethyl-1H-indazol-6-yl)-1,2,4-oxadiazol-5-yl)piperidin-1-yl)-2-oxoethyl)benzamide (46 mg, 101 μmol, 25%) as a yellow solid. ¹H NMR (400 MHz, CDCl₃) δ 8.02 (s, 1H), 7.81-7.73 (m, 3H), 7.66 (dd, J=0.6, 8.4 Hz, 1H), 7.48-7.42 (m, 1H), 7.42-7.35 (m, 2H), 7.26 (br. s., 1H), 4.46 (d, J=14.1 Hz, 1H), 4.24 (d, J=3.9 Hz, 2H), 4.01 (s, 3H), 3.86 (d, J=13.7 Hz, 1H), 3.29 (ddd, J=3.6, 10.5, 14.2 Hz, 2H), 3.13-3.04 (m, 1H), 2.53 (s, 3H), 2.26-2.15 (m, 2H), 2.04-1.89 (m, 2H); LCMS (ESI) m/z: [M+H]⁺=459.3.

Compound 5 has the structure:

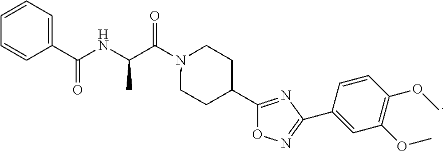 [SMILES: COc1ccc(-c2noc(C3CCN(C(=O)[C@@H](C)NC(=O)c4ccccc4)CC3)n2)cc1OC]

Compound 5 may be synthesized by methods known in the art. For example, Compound 5 may be synthesized as shown in the scheme below:

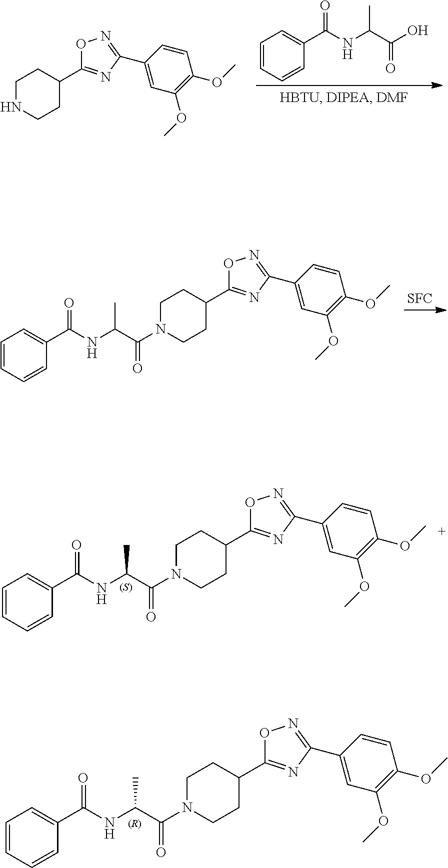

### Step 1: Preparation of N—[(R)-2-[4-[3-(3,4-dimethoxyphenyl)-1,2,4-oxadiazol-5-yl]-1-piperidyl]-1-methyl-2-oxo-ethyl]benzamide and N—[(S)-2-[4-[3-(3,4-dimethoxyphenyl)-1,2,4-oxadiazol-5-yl]-1-piperidyl]-1-methyl-2-oxo-ethyl]benzamide

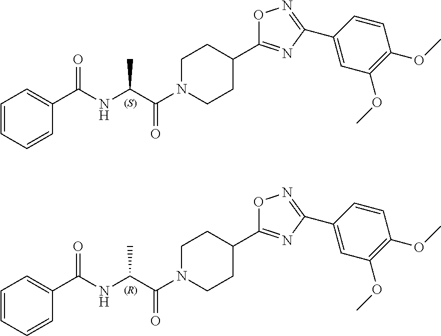

To a stirred solution of 3-(3,4-dimethoxyphenyl)-5-(4-piperidyl)-1,2,4-oxadiazole (150 mg, 518 μmol) and 2-benzamidopropanoic acid (105 mg, 544 μmol) in N,N-dimethylformamide (2 mL) was added (2-(1H-benzotriazol-1-yl)-1,1,3,3-tetramethyluronium hexafluorophosphate) (196 mg, 518 μmol) and N-ethyl-N-(propan-2-yl)propan-2-amine (201 mg, 1.56 mmol, 271 μL). The mixture was stirred at 20° C. for 5 h. The crude product was purified by prep-HPLC (column: Luna C18 150×25 5 μm; mobile phase: [water (10 mM ammonium carbonate)-acetonitrile]; B %: 35%-65%,12 min) to give rac-N-(1-(4-(3-(3,4-dimethoxyphenyl)-1,2,4-oxadiazol-5-yl)piperidin-1-yl)-1-oxopropan-2-yl)benzamide then the product purified by SFC separation (column: AD(250×30 mm, 5 μm); mobile phase: [Neu-IPA]; B %: 42%-42%, min) to give N-[2-[4-[3-(3,4-dimethoxyphenyl)-1,2,4-oxadiazol-5-yl]-1-piperidyl]-1-methyl-2-oxo-ethyl]benzamide, Enantiomer 1 (63 mg, 134.93 μmol, 26%) as a white solid and N-[2-[4-[3-(3,4-dimethoxyphenyl)-1,2,4-oxadiazol-5-yl]-1-piperidyl]-1-methyl-2-oxo-ethyl]benzamide, Enantiomer 2 (56 mg, 120 μmol, 23% as a white solid.

### N-[2-[4-[3-(3,4-dimethoxyphenyl)-1,2,4-oxadiazol-5-yl]-1-piperidyl]-1-methyl-2-oxo-ethyl]benzamide, Enantiomer 1

¹H NMR (400 MHz, DMSO-d₆) δ=8.63 (br dd, J=7.3, 16.1 Hz, 1H), 7.88 (br d, J=7.5 Hz, 2H), 7.62-7.41 (m, 5H), 7.11 (br d, J=8.2 Hz, 1H), 4.97 (br d, J=6.4 Hz, 1H), 4.43-4.24 (m, 1H), 4.10-3.95 (m, 1H), 3.82 (s, 6H), 3.42 (br t, J=10.8 Hz, 1H), 3.30-3.21 (m, 1H), 2.99-2.83 (m, 1H), 2.09 (br d, J=11.9 Hz, 2H), 1.83-1.60 (m, 2H), 1.30 (br s, 3H); LCMS (ESI) m/z: [M+H]⁺=465.3. ee=100%.

### N-[2-[4-[3-(3,4-dimethoxyphenyl)-1,2,4-oxadiazol-5-yl]-1-piperidyl]-1-methyl-2-oxo-ethyl]benzamide, Enantiomer 2

¹H NMR (400 MHz, DMSO-d₆) δ=8.65 (br dd, J=7.6, 16.1 Hz, 1H), 7.98-7.86 (m, 2H), 7.70-7.41 (m, 5H), 7.13 (br d, J=8.2 Hz, 1H), 5.00 (br d, J=5.5 Hz, 1H), 4.49-4.24 (m, 1H), 4.12-3.96 (m, 1H), 3.85 (s, 6H), 3.45 (br t, J=10.7 Hz, 1H), 3.27 (br s, 1H), 3.05-2.83 (m, 1H), 2.12 (br d, J=12.5 Hz, 2H), 1.89-1.61 (m, 2H), 1.32 (br s, 3H); LCMS (ESI) m/z: [M+H]⁺=465.3. ee=99.6

Compound 6 has the structure:

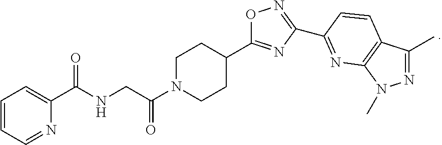 [SMILES: Cc1nn(C)c2nc(-c3noc(C4CCN(C(=O)CNC(=O)c5ccccn5)CC4)n3)ccc12]

Compound 6 may be synthesized by methods known in the art. For example, Compound 6 may be synthesized as shown in the scheme below:

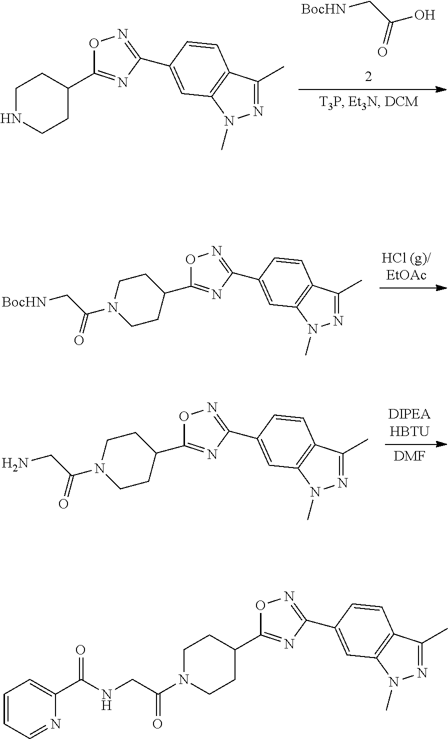

¹H NMR (400 MHz, CDCl₃) δ 8.93 (br s, 1H), 8.63 (d, J=4.0 Hz, 1H), 8.19 (d, J=7.7 Hz, 1H), 8.10 (s, 1H), 7.89-7.80 (m, 2H), 7.76-7.71 (m, 1H), 7.47-7.41 (m, 1H), 4.56 (br d, J=13.7 Hz, 1H), 4.35 (d, J=4.4 Hz, 2H), 4.09 (s, 3H), 3.96 (br d, J=13.9 Hz, 1H), 3.44-3.31 (m, 2H), 3.15 (br t, J=10.7 Hz, 1H), 2.60 (s, 3H), 2.34-2.23 (m, 2H), 2.11-1.95 (m, 2H); LCMS (ESI) m/z: [M+H]⁺=460.2.

Compound 7 has the structure:

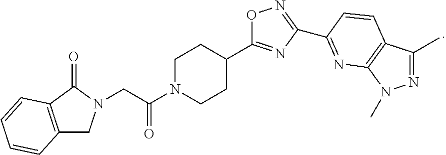 [SMILES: Cc1nn(C)c2nc(-c3noc(C4CCN(C(=O)CN5Cc6ccccc6C5=O)CC4)n3)ccc12]

Compound 7 may be synthesized by methods known in the art. For example, Compound 7 may be synthesized as shown in the scheme below:

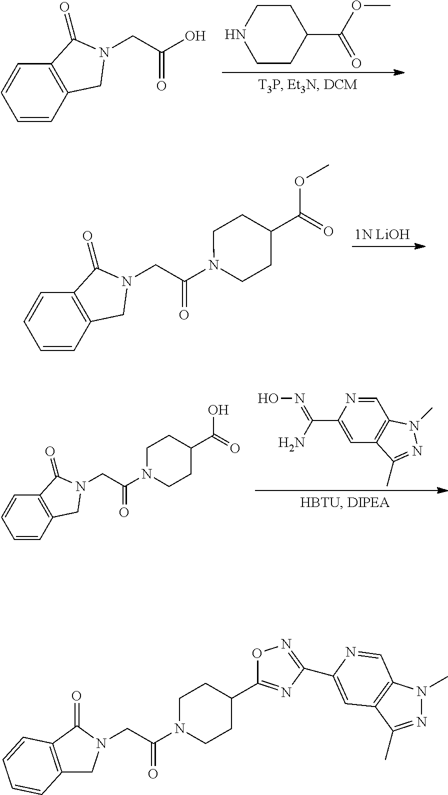

¹H NMR (400 MHz, CHLOROFORM-d) δ=8.11 (d, J=8.4 Hz, 1H), 7.89 (d, J=8.2 Hz, 1H), 7.85 (d, J=8.2 Hz, 1H), 7.56-7.50 (m, 1H), 7.47-7.41 (m, 2H), 4.60-4.41 (m, 5H), 4.16 (s, 3H), 4.12-4.05 (m, 1H), 3.41-3.31 (m, 2H), 3.06-2.98 (m, 1H), 2.62-2.57 (m, 3H), 2.25 (br t, J=14.6 Hz, 2H), 2.05-1.94 (m, 2H); LCMS(ESI) m/z: [M+H]⁺:472.3.

Drug Resistant Mutant Selection

Strains GMYF and W-erg3 were grown to saturation in CSM-glucose, centrifuged, resuspended in phosphate-buffered Saline (PBS), and plated at a density of 10⁷ cells/plate on solid 15 cm petri dishes containing CSM with 2% galactose (w/v), 2% (w/v) agar, and 10 μM Compound 3, and incubated at 30° C. Resistant colonies were isolated after 5-7 days, re-streaked on the same media, and resistance reconfirmed. Cultures of validated strains were then inoculated for genomic DNA isolation using a YeaStar™ yeast genomic DNA kit (Zymo Research).

Libraries were prepared for sequencing using the Illumina NEXTERA™ library prep kit and sequenced via Illumina Hiseq™ 2500 1×50 bp (single end reads). Sequences were aligned to the S. cerevisiae reference genome (S288CCR64-1-1, Saccharomyces Genome Database (SGD)) using Burrows-Wheeler Aligner (BWA, see, e.g., Li et al. Bioinformatics 25:1754-1760, 2009; Li et al. Bioinformatics 2010, Epub (PMID 20080505)). The BWA output SAI files were converted to SAM files using BWA. The SAM files were sorted using SAMtools 1.3.1 (Li et al. Bioinformatics 25:2079-2079, 2009). Variants (single-nucleotide polymorphisms (SNPs), indels) were identified using Freebayes (see, e.g., arXiv:1207.3907). Variant locations were summarized using snpEFF (Cingolani et al. Fly (Austin) 6(2):80-92, 2012).

Quantitative Lipid Profiling

Overnight cultures of yeast strain W303 pdr1Δ pdr3Δ were diluted into CSM media with 2% (w/v) raffinose, OD₆₀₀ 0.25, and grown for 4 h before resuspending at an OD₆₀₀ of 0.2 in CSM media with 2% (w/v) galactose and adding Compound 2 or DMSO at the indicated concentrations. Cells were grown for the indicated timepoints before centrifugation, washing once in PBS, and freezing pellets. Lipids were extracted from pellets by resuspending the pellets in 600 μL methanol, 300 μL water, and 400 μL chloroform, followed by cell lysis by vortexing with glass beads for 1 min. Samples were then centrifuged at 10,000×g for 10 min, and the bottom layer that formed (organic/lipids) was moved into a new tube and evaporated. Samples were then analyzed by LC/MS/MS using a Thermo Scientific Q Exactive™ Orbitrap™ coupled to a Dionex UltiMate® 3000 ultra-high performance liquid chromatography system, following the method described in Tafesse et al. PLoS Pathog. 11(10): e1005188, 2015.

### B. Results

The effect of 1,2,4-oxiadiazoles on cell growth was assessed in a control condition and in a yeast model for ApoE4 toxicity (see International Patent Application Publication No. WO 2016/040794). The control condition was growth of the ApoE4 strain under non-inducing conditions using raffinose as the carbon source. The 1,2,4-oxadiazoles exhibited a bell-shaped rescue curve in the ApoE4 model (FIG. 2A, top panel). At higher concentrations, these compounds inhibited the growth in the control condition (FIG. 2B, bottom panel). The potency of model rescue correlated well with the potency of growth inhibition across the entire series of 1,2,4-oxadiazoles tested (FIG. 2B). These relationships indicate that the growth inhibition arises from an “on-target” activity, i.e., over activation or inhibition of a target that results in slowed growth.

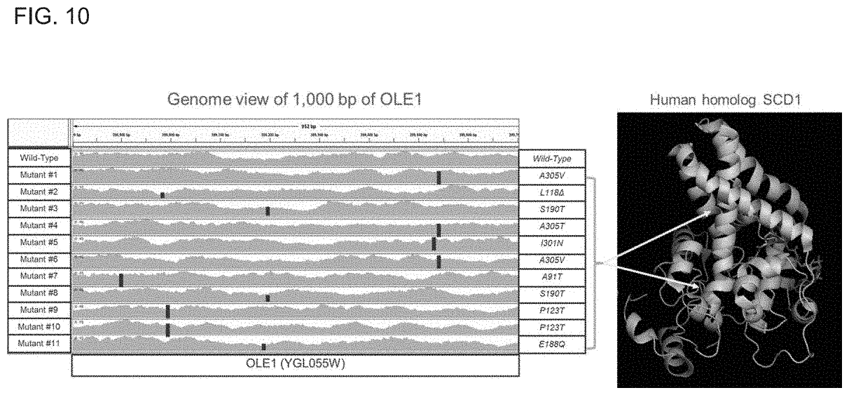

Drug-resistant mutants can be used to identify the target of the compounds, for example, by preventing or reducing drug binding, and therefore allowing growth under inhibitory doses of 1,2,4-oxadiazole concentrations. Twenty drug-resistant mutants were isolated, and the mutants were subjected to whole-genome sequencing in order to identify genetic lesions associated with the drug resistance. Surprisingly, all mutations identified in the drug resistant mutants localized to OLE1 (YGL055W), the sole stearoyl-CoA desaturase (SCD; also referred to as A9-desaturase) in yeast (FIG. 10). The drug resistant mutants specifically conferred resistance to 1,2,4-oxadiazoles, but were not cross-resistant to other toxic compounds. The ole1 mutations identified included indels and substitution mutations, including A305V, L118Δ, S190T, A305T, 1301 N, A91T, S190T, P123T, and E118Q. These mutations are relative to the wild-type OLE1 sequence provided below.

(SEQ ID NO: 1)
MPTSGTTIELIDDQFPKDDSASSGIVDEVDLTEANILATGLNKKAPRIVN
GFGSLMGSKEMVSVEFDKKGNEKKSNLDRLLEKDNQEKEEAKTKIHISEQ
PWTLNNWHQHLNWLNMVLVCGMPMIGWYFALSGKVPLHLNVFLFSVFYYA
VGGVSITAGYHRLWSHRSYSAHWPLRLFYAIFGCASVEGSAKWWGHSHRI
HHRYTDTLRDPYDARRGLWYSHMGWMLLKPNPKYKARADITDMTDDWTIR
FQHRHYLLMLLTAFVIPTLICGYFFNDYMGGLIYAGFIRVFVIQQATFCI
NSLAHYIGTQPFDDRRTPRDNWITAIVTFGEGYHNFHHEFPTDYRNAIKW
YQYDPTKVIIYLTSLVGLAYDLKKFSQNAIEEALIQQEQKKINKKKAKIN
WGPVLTDLPMWDKQTFLAKSKENKGLVIISGIVHDVSGYISEHPGGETLI
KTALGKDATKAFSGGVYRHSNAAQNVLADMRVAVIKESKNSAIRMASKRG
EIYETGKFF

These data strongly suggest that Ole1 is the target of 1,2,4-oxadiazoles. Additionally, addition of exogenous oleic acid reversed both growth inhibition of wild-type cells and rescue of toxicity in a yeast disease model of alpha-synuclein toxicity (FIGS. 3A and 3B, respectively). Likewise, these effects were specific for 1,2,4-oxadiazoles, but not other toxic compounds.

Drug-resistant Ole1 mutations reduced 1,2,4-oxadiazole-induced growth inhibition in wild-type cells (FIG. 4A). The same mutations also increased the EC50 (concentration that gives half-maximal response) in the context of the alpha-synuclein model, which is consistent with reduced binding to the target. These shifts in does response were specific for 1,2,4-oxadiazoles. These data further support that Ole1/SCD is the target for both growth inhibition and rescue of toxicity in disease models.

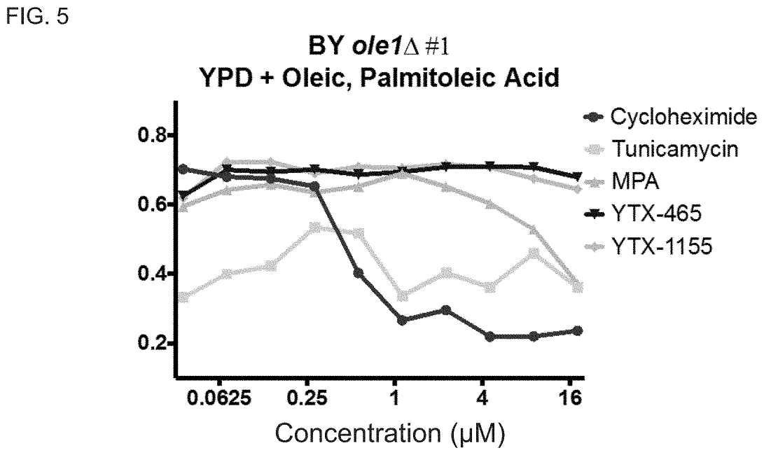

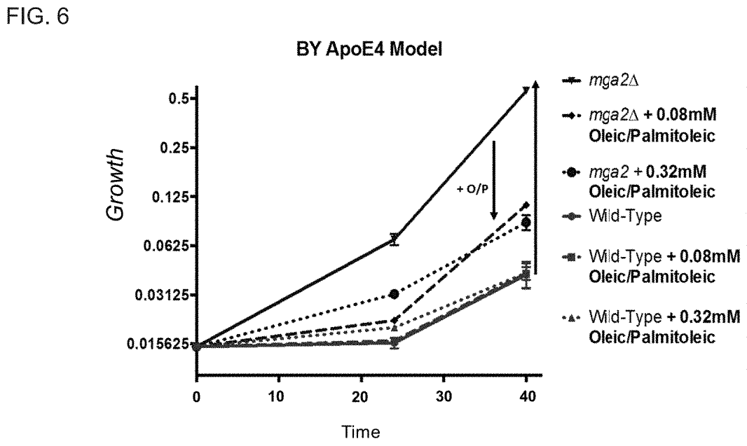

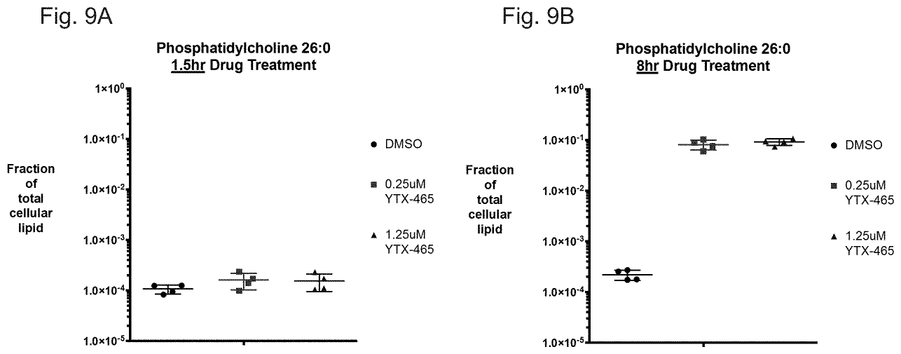

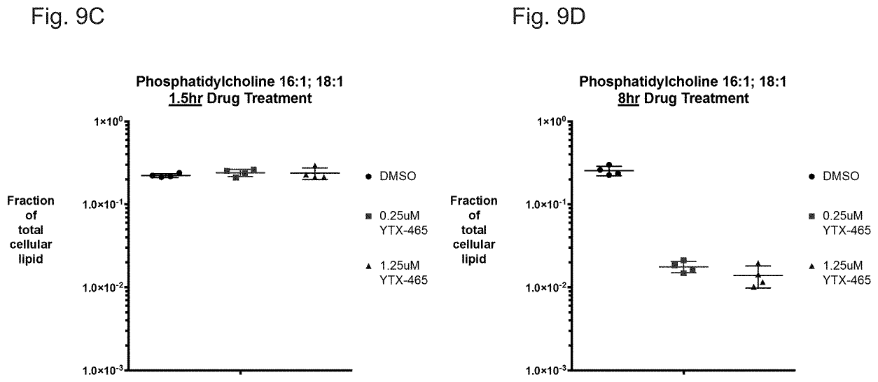

The OLE1 gene is essential in Saccharomyces cerevisiae. However, strains deleted for OLE1 (ole1A) are viable if their growth media is supplemented with oleic/palmitoleic acid. The ole1Δ strain supplemented with exogenous fatty acids was fully resistant to 1,2,4-oxadiazoles (FIG. 5). In other words, in the absence of the target, Ole1, the 1,2,4-oxadiazoles do not have growth inhibition activity. Independently, a chemical genetics approach identified MGA2, the transcription factor that regulates Ole1. Genetic deletion of MGA2 (mga2A) phenocopied the effects of 1,2,4-oxadiazoles (FIG. 6). mga2A cells have reduced Ole1 levels, which itself rescues toxicity in the yeast disease models (e.g., the ApoE4 model). Supplementation of the growth media with oleic acid reversed this effect, similar to the results described above. Consistent with these data, treatment of yeast cells with the 1,2,4-oxadiazole Compound 2 inhibited lipid desaturation (FIGS. 9A-9D). Overall, these data provide still further evidence that Ole1/SCD is the target of 1,2,4-oxadiazoles.

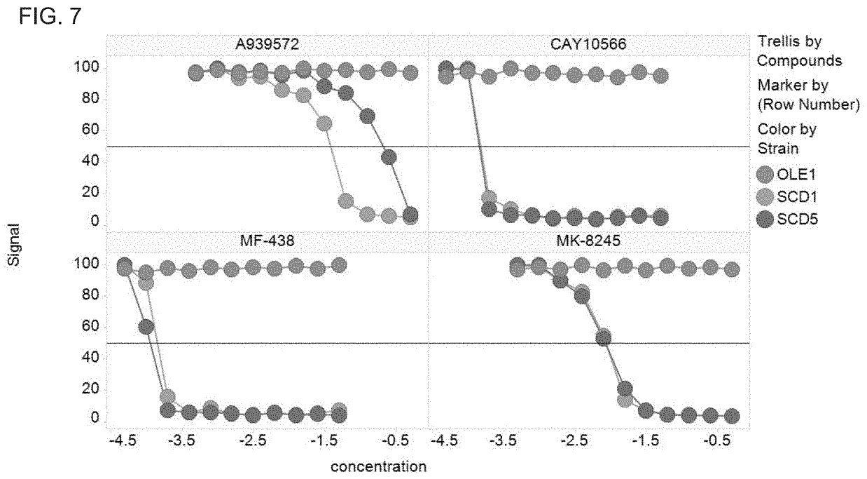

Humanized yeast strains expressing the human SCD proteins SCD1 or SCD5 were generated by genetic deletion of OLE1 and expressing human SCD1 or SCD5 on a plasmid. Yeast expressing OLE1 were resistant to known SCD1/SCD5 inhibitors such as A939572, CAY10566, MF-438, and MK-8245 (FIG. 7), suggesting that they do not target the yeast enzyme. In marked contrast, in the SCD1 and SCD5 humanized strains, the known SCD1/SCD5 inhibitors were extremely potent, with low nanomolar half-maximal inhibitory concentration (IC₅₀) values (FIG. 7).

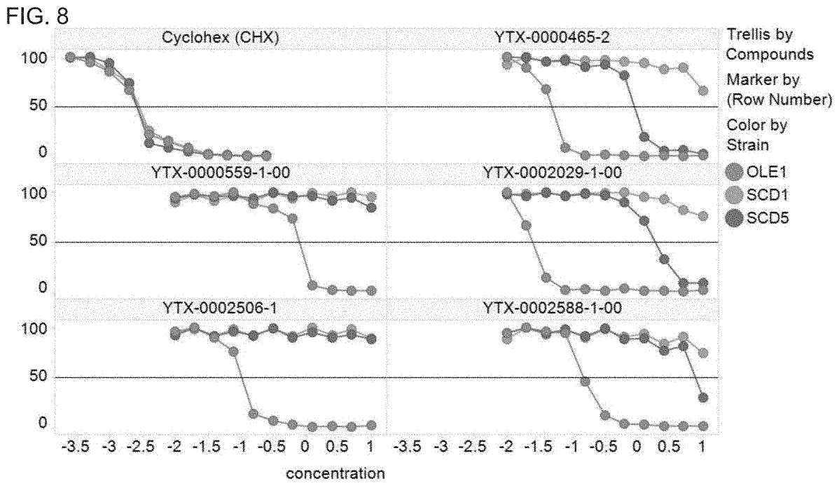

The effect of 1,2,4-oxadiazoles was also evaluated in both of the humanized SCD1 and SCD5 models. 1,2,4-oxadiazoles inhibited the growth of the SCD1 and/or SCD1 yeast strains, and differences in the structure-activity relationship (SAR) between the three SCD proteins was observed (FIG. 8). Some compounds inhibited the growth of both the SCD1 and the SCD5 strains. Other compounds appeared to target only the yeast enzyme. Out of a total of 250 1,2,4-oxadiazoles tested, 117 compounds exhibited significant activity (e.g., greater than 50% inhibition of growth) against the human enzymes, i.e., SCD1 and/or SCD5. The divergent SAR provides additional strong evidence for SCD being the target of 1,2,4-oxadiazoles.

Finally, treatment of yeast cells with the 1,2,4-oxadiazole Compound 2 inhibited lipid desaturation (FIGS. 9A-9D), providing additional confirmatory evidence that SCD is the target of 1,2,4-oxadiazoles.

Taken together, these data demonstrate that Ole1/SCD is the target of 1,2,4-oxadiazoles, and that these compounds inhibit Ole1/SCD. Further, these data show that inhibition of Ole1/SCD rescues cell toxicity associated with expression of neurological disease proteins in yeast models, including ApoE4 and alpha-synuclein models, suggesting that SCD inhibition as a therapeutic approach for neurological disorders including Alzheimer's disease and Parkinson's disease.

### Example 3. A Decrease in Desaturated Fatty Acids Rescues Alpha-Synuclein-Dependent Cell Toxicity, Neurite Degeneration, and Neuronal Cell Death

### A. Materials and Methods

Molecular Biology and Compound Sources

Expression constructs for alpha-synuclein wild-type and A53T (SNCA), empty vector controls (pcDNA, pCAGGs), and mRab1a were obtained from the Whitehead Institute (Massachusetts Institute of Technology, Cambridge, Mass.). The pSF-CAG plasmid was obtained from Oxford Genetics (Oxford, UK). The red fluorescent protein (RFP) reporter plasmid, pSF-MAP2-mApple, was constructed by replacing the CAG promoter with human MAP2 promoter sequence, and inserting mApple coding sequence into the multiple cloning site. The RFP reporter plasmid, pSF-CAG-mKate2, was generated by inserting the mKate2 coding sequence into pSF-CAG plasmid by PCR assembly. CAY10566 was purchased from Abcam. “SMARTpool” siRNAs for SCD1 and SCD5 were purchased from GE Dharmacon.

Cell Culture

U2OS cells (Sigma-Aldrich) between passages 12 to 22 were cultured in McCoy's 5A medium (ATCC) supplemented with 10% heat inactivated fetal bovine serum (Thermo Fisher). Induced pluripotent stem cells (iPSC)-derived neurons containing a triplication in the SCNA gene (S3) were maintained in brain-derived neurotrophic factor (BDNF), cyclic adenosine monophosphate (cAMP), and glial cell-line derived neurotrophic factor (GDNF)-supplemented growth medium as previously described (Chung et al. Science 342(6161):983-987, 2013). Four weeks after cells were differentiated into neurons, cells were harvested and RNA was extracted. PC12 cells (ATCC) were cultured in F12K medium supplemented with 15% horse serum and 2.5% fetal bovine serum (Thermo Fisher). RNA extracted from the rat PC12 cells (passage 22) was used as a negative control for the expression of SCD1 and SCD5.

RNA Purification and Reverse Transcription-Polymerase Chain Reaction (RT-PCR)

Cells (iPSC-derived neurons, PC12 and U2OS) were rinsed with ice-cold PBS (pH 7.4). Total RNA was purified using an RNEasy@ Mini Kit following the manufacturer's instructions (Qiagen). Reverse transcription was performed with 150 ng RNA using a High-Capacity cDNA Reverse Transcription Kit (Thermo Fisher) in a MASTERCYCLER® Pro thermal cycler (Eppendorf). Real-time PCR analyses of 2 μL cDNA products in a total reaction volume of 20 μL were carried out in duplicates using TaqMan® Fast Advanced Master Mix in a StepOnePlus™ Real-Time PCR System (Thermo Fisher). The primer pairs and probes for real-time amplification of SCD1 and SCD5 were predesigned TaqMan® gene expression assays (Applied Biosystems # Hs01682761_m1 and # Hs00227692_m1, respectively). Human beta-actin was used as an endogenous housekeeping control (Applied Biosystems #4310881E). The relative quantity of gene transcript abundance was calculated using the ΔΔCt method.

Western Immunoblotting

Cells were rinsed with ice-cold PBS and lysed in ice-cold radioimmunoassay precipitation buffer (RIPA, Thermo Fisher) containing protease and phosphatase inhibitor cocktails (Sigma-Aldrich) for 15 min on ice. The lysates were centrifuged at 10,000×g for 10 min at 4° C. Supernatant was collected and protein concentrations were measured using a bicinchoninic acid (BCA) kit (Pierce). Ten micrograms of total protein were resolved in 4-12% NuPAGE® Bis-Tris gels (Thermo Fisher) by electrophoresis then transferred to nitrocellulose membranes using the iBlot® system (Thermo Fisher). Membranes were blocked in 1:1 dilution of ODYSSEY® blocking buffer (LI-COR Biosciences) and PBS for 1 h at room temperature followed by incubation with primary anti-SCD1 (1/1000 dilution, Abcam) and anti-3-tubulin (1/4000 dilution, Sigma-Aldrich) antibodies in blocking buffer containing 0.1% of TWEEN®-20 at 4° C. overnight with gentle rocking. After three washes with PBS plus 0.1% TWEEN®-20 (PBST), blots were incubated with secondary antibodies conjugated to IRDye® 680 or 800 (1:8,000, Rockland Immunochemicals) in blocking buffer for 2 hours at room temperature. After three washes with PBST and two with water, blots were scanned in an ODYSSEY® quantitative fluorescent imaging system (LI-COR Biosciences).

U2OS Cell Transfection

U2OS cells were trypsinized using 0.25% trypsin-EDTA (Thermo Fisher) for 5 min at 37° C. followed by centrifugation at 800 rpm for 5 min at room temperature. Cell pellets were re-suspended in SE solution (Lonza Biologics, Inc.) at a density of 1×10⁴ cells/IL. Alpha-synuclein wild-type or empty control (pcDNA) plasmids were transfected at a ratio of 10 mg per 1,000,000 cells. For genetic modifier studies, mRab1a was titrated at various concentrations in the presence of SNCA plasmids. Nucleofection was performed using 4D-NUCLEOFECTOR™ System (Lonza Biosciences, Inc.) under program code CM130 in either 20 μL Nucleocuvette™ strips or 100 μl single Nucleocuvettes™. Cells recovered at room temperature for 10-15 minutes after nucleofection before further handling. Pre-warmed medium was added and cells were thoroughly but gently mixed to a homogenous suspension before plating. Cells were seeded at 2×10⁴ cells/100 μl/well into 96 well PLD-coated white plates (Corning, Inc.) using a customized semi-automated pipetting program (VIAFLO 384/96, Integra Biosciences).

U2OS ATP Assay

Powders of reference SCD inhibitors (CAY10566, A939572 and MF-438) were resuspended and serial diluted in DMSO. Compound treatment solutions were then prepared in complete U2OS growth medium such that compounds were held at 6-fold higher than the final intended treatment concentration. At 4 h after nucleofection, 20 μL of the 6× compound solutions were then added to wells containing SNCA transfected cells and 100 μL growth media. The final DMSO concentration was 0.3%. Plates were gently rocked to mix the drug solution into well media, and plates were incubated for 72 h with the compounds. Plates were sealed with MicroClime® lids (Labcyte Inc.) to reduce evaporation and variability. ATP content was then measured using the CellTiter-Glo® kit (Promega) with luminescence signals measured on an EnVision multimode plate reader (Perkin Elmer).

Primary Neuron Transfections

Rat primary cortical neurons cultured in 96-well plates (Greiner Bio-One) were co-transfected with a fluorescence reporter plasmid (encoding mKate2) and empty or alpha-synuclein-A53T overexpression plasmids by lipofection at 5-6 div (days in vitro). LIPOFECTAMINE® 2000 transfection reagent (Thermo Fisher) (0.5 μl/well) was diluted in NEUROBASAL® media (Thermo Fisher) and incubated for 5-10 min. The LIPOFECTAMINE®/NEUROBASAL® mixture was then added dropwise to a plasmid cocktail diluted in NEUROBASAL® media, and incubated for approximately 40 min. During this time, conditioned media on the neurons was replaced with media containing 1× kynurenic Acid (Sigma-Aldrich) in NEUROBASAL® media (NBKY). LIPOFECTAMINE®/DNA complex solutions were subsequently added dropwise to neurons in the NBKY media in the 96-well plate. Lipofection was carried out for 30-40 min in a standard cell culture incubator (37° C., 5% CO₂). Neurons were then washed with NEUROBASAL® media, and 50% conditioned/50% fresh NEUROBASAL® media containing B-27 supplement and GlutaMax™ (Thermo Fisher) (NBM) was applied to the cultures.

Human control and patient-derived trans-differentiated neurons were transfected with an RFP reporter driven by the human MAP2 promoter (MAP2-mApple) following the protocol for rat primary neurons as described above with the following exceptions: lipofection was carried out for approximately 1 h, and the final media replacement was with BrainPhys™ media supplemented with BDNF, GNDF, cAMP, ascorbic acid, and laminin.

Neurite Degeneration Assay

Transfected rat cortical neuron cultures were treated with DMSO or CAY10566 compound 4-6 h post-transfection. Vehicle or compound were diluted in NBM at the indicated concentrations. Culture plates were imaged at 6 h intervals in the IncuCyte® ZOOM (Essen Bioscience) incubator/imaging system for approximately 1 week. Neurite lengths of transfected neurons were tracked by an RFP reporter, mKate2, and measured by NeuroTrack™ Software Module (Essen Bioscience). Neurite lengths were normalized to the peak neurite length for each transfection group (6 replicate wells) and plotted to assess the neurite degeneration phase.

Neuron Survival Assay

Transfected neuronal cultures were imaged at 12-24 h intervals for the indicated number of days by robotic microscopy. Fluorescence images were acquired with a Nikon Eclipse Ti microscope equipped with a motorized stage, 20× extra-long working distance (ELWD) objective, and an Andor Zyla cMOS camera. During image acquisition, microplates were enclosed in an on-stage environmental chamber controlling temperature, CO₂, and humidity (Okolab). Images were processed and analyzed with custom-made scripts in R and ImageJ software. The lifetimes of individual neurons were determined by tracking fluorescently-labeled neurons in ImageJ. Neuronal death was determined to occur upon incidence of RFP signal loss or rupture of cell body. Cox proportional hazards analysis was used to generate cumulative hazard plots and determine the risk of neuron death. Log-rank test was used to determine statistical significance of survival curve divergence between neuron cohorts.

### B. Results

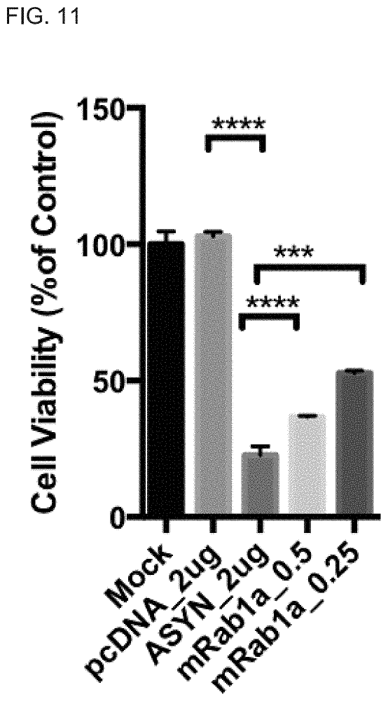

To investigate the cellular events related to alpha-synuclein pathology, an assay was developed to measure the effects of alpha-synuclein expression on cellular ATP content in transfected U2OS cells, which is a general proxy for cell health and viability. U2OS cells transfected with alpha-synuclein exhibited a significant reduction in cellular ATP levels relative to cells transfected with the “empty” pCDNA vector control (FIG. 11). To evaluate the relevance of this alpha-synuclein-dependent decrease in ATP levels, U2OS were co-transfected with alpha-synuclein and mammalian Rab1a (mRab1a, a Rab GTPase family member), which is a known genetic modifier of alpha-synuclein toxicity in neurons and is involved in intracellular vesicle trafficking (Cooper et al. Science 313(5785):324-328, 2006). Co-transfecting mRab1a into U2OS cells with alpha-synuclein demonstrated that cellular ATP levels were significantly higher in co-transfected cells as compared to alpha-synuclein alone. This rescue of alpha-synuclein toxicity is reminiscent of that which occurs in neurons, indicating that the alpha-synuclein-dependent decrease of ATP content in U2OS cells may be recapitulating similar cellular pathological events. This indicates the U2OS model is useful for evaluating alpha-synuclein biology and toxicity.

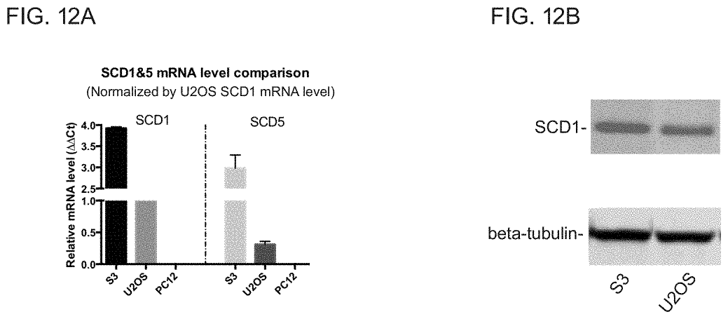

Humans are known to express two different isoforms of stearoyl-CoA desaturase, SCD1 and SCD5 (Wang et al., Biochem. Biophys. Res. Commun. 332(3):735-42, 2005). SCD1 and SCD5 transcript levels were first evaluated by RT-PCR to determine whether the human U2OS cell line could be used to characterize the effects of SCD inhibitors. Analysis of mRNA isolated from U2OS cells demonstrated that this cell line expressed measurable levels of both SCD1 and SCD5, with approximately 4-fold higher relative levels of SCD1 (FIG. 12A). As a positive control for the SCD1 and SCD5 RT-PCR probe sets, RNA extracted from human iPSC-derived neurons containing a triplication of the alpha-synuclein gene (S3 neurons) was also analyzed, as human brain samples have previously been shown to express both SCD1 and SCD5 (Wang et al., supra). Similar to published results, cultures of human S3 neurons were found to express both SCD1 and SCD5, with approximately 25% higher expression of SCD1. RNA extracts prepared from rat PC12 cells demonstrated the specificity of the human probe sets, as no significant amplification was detected in these samples.

To confirm and extend the RT-PCR results, cell extracts from S3 neurons and U2OS cells were analyzed for expression of SCD1 protein by Western immunoblotting. This analysis confirmed that both cell populations expressed SCD1 at similar levels, relative to a beta-tubulin loading control (FIG. 12B). Attempts to measure SCD5 protein in these cell preparations were unsuccessful, as the commercially available antibody appeared unsuitable for this purpose.

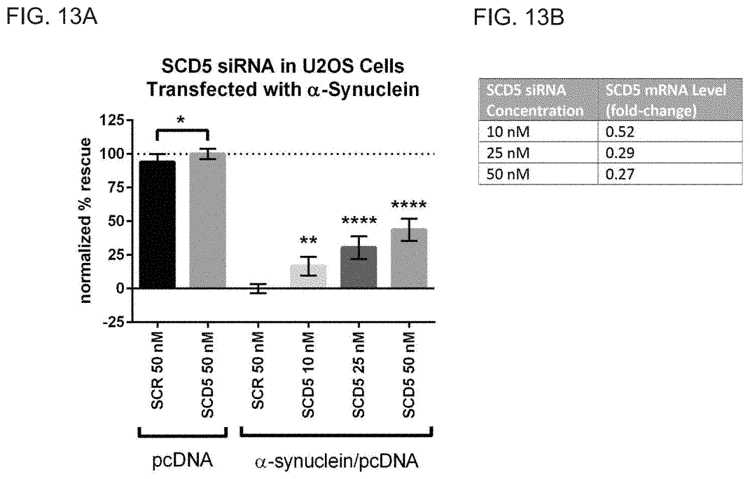

The potential role of SCD in mediating alpha-synuclein-induced toxicity in U2OS cells was evaluated by siRNA knockdown of SCD1 and SCD5 expression. U2OS cells were transfected with empty vector controls, or the same plasmid containing alpha-synuclein. Cells were also co-treated with either a control scrambled siRNA, or siRNAs against human SCD1 or SCD5. Cells treated with SCD1 siRNA exhibited a general increase in ATP levels in either the presence or absence of alpha-synuclein. Thus, a specific role of SCD1 in mediating alpha-synuclein toxicity could not be evaluated under these experimental conditions. However, SCD5 knockdown resulted in a concentration-dependent rescue, which inversely correlated with levels of SCD5 mRNA (FIGS. 13A and 13B), suggesting that decreasing SCD5 transcript, and subsequently protein and activity, provided a beneficial effect.

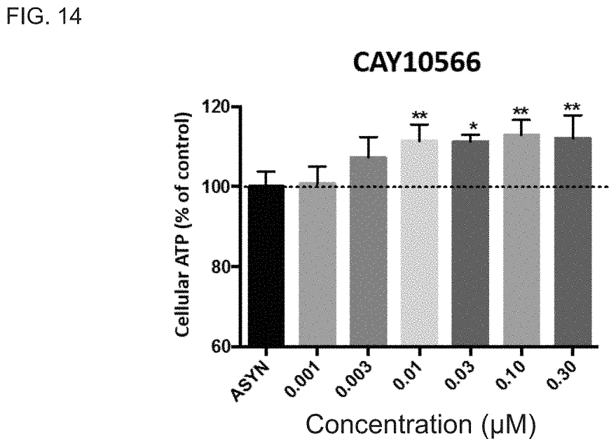

To further investigate a potential role of SCD in mediating alpha-synuclein cell toxicity, U2OS cells transfected with alpha-synuclein were also treated with a titration of a commercially available SCD inhibitor (CAY10566). ATP levels were assessed 72 h after treatment. CAY10566 significantly reversed alpha-synuclein-dependent decreases in ATP levels in a concentration-dependent fashion (FIG. 14). These data indicate that inhibiting SCD activity in U2OS cells ameliorated the pathological effects of alpha-synuclein on overall cellular health.

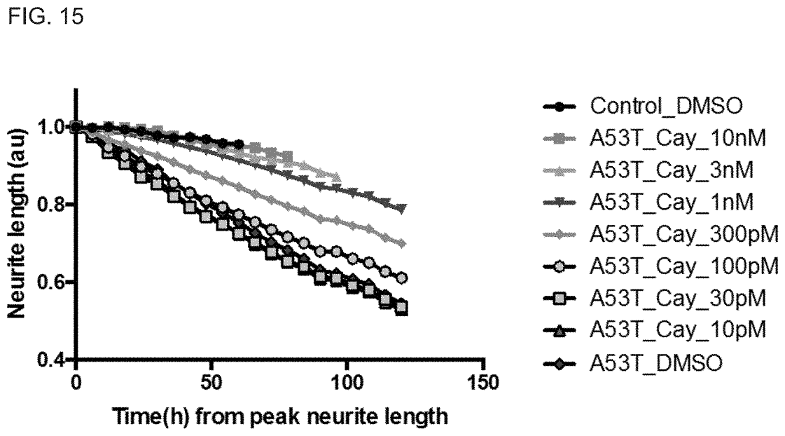

The role of SCD in mediating alpha-synuclein-dependent pathological process was next investigated in a more relevant neuronal system. Primary cultures of rat cortical neurons were transfected with α-synuclein containing the A53T mutation and also treated with a titration of CAY10566. Neurite length was measured in live cells every 6 hours after transfection for a total of 7 days. Transfected cells were tracked with a fluorescent reporter (mCherry). Relative to DMSO controls, cells transfected with α-synuclein and treated with CAY10566 exhibited a concentration-dependent decrease in neurite degeneration (FIG. 15). Cells treated with the highest concentrations of CAY10566 (10 nM and 3 nM) exhibited slower neurite degeneration that was overlapping with control cultures that were not transfected with alpha-synuclein A53T, suggesting a complete rescue of alpha-synuclein detrimental effects. These data indicate that inhibition of SCD activity with CAY10566 was sufficient to reduce the pathological effects of alpha-synuclein overexpression on neurite degeneration.

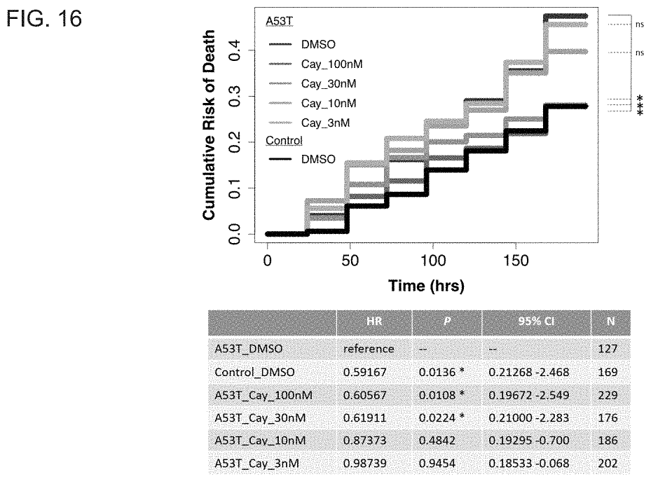

To evaluate the effects of SCD inhibition in human neurons, human iPSC cells harboring the alpha-synuclein A53T mutation or an isogenic control line in which the A53T mutation was corrected to wild-type, were trans-differentiated into neurons, and cell survival was monitored over the course of 8 to 10 d. Analysis of cumulative single cell survival data indicated that the risk of neuron death was significantly reduced by treatment with CAY10566 at 100 nM and 30 nM (FIG. 16) relative to DMSO controls in the A53T neurons. Interestingly, at these concentrations of CAY10566, the risk of cell death was reduced back to levels observed in the isogenic control neurons, suggesting the enhanced toxicity of alpha-synuclein A53T on cell viability was eliminated.

Taken together, these data demonstrate that a decrease in desaturated fatty acids by SCD1 and/or SCD5 inhibition rescues a number of phenotypes associated with neurological diseases in relevant disease models, providing further evidence that a decrease in desaturated fatty acids by SCD inhibition as a therapeutic approach for neurological diseases including Alzheimer's disease and Parkinson's disease.

### Example 4. A Decrease in Desaturated Fatty Acids Reduces Risk of Neuron Death from α-Synuclein Toxicity and Result in Pharmacodynamic Responses in the Brain

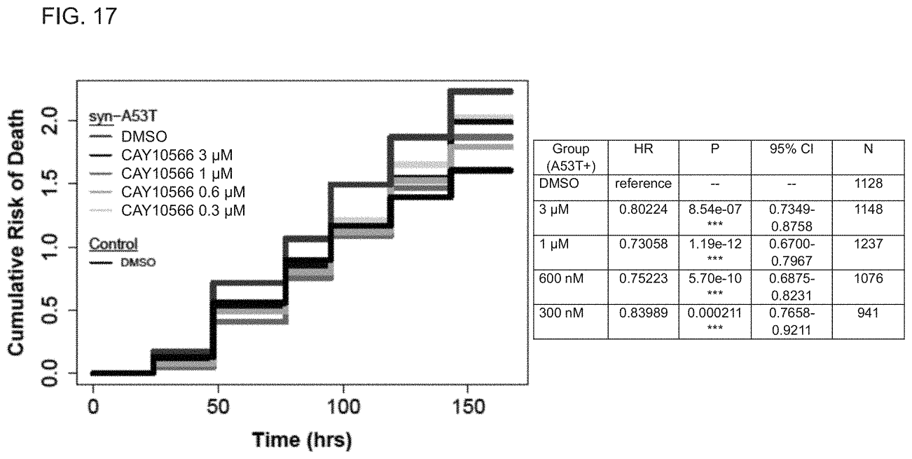

A model of α-synuclein toxicity utilizing transient transfection into human iPSC-derived neurons was developed. In response to α-synuclein transfection, human neurons exhibit a significantly increased risk of death that can be tracked in live cells over the course of several days. This model was utilized to evaluate the role of SCD in α-synuclein-dependent neuronal toxicity. Human iPSC-derived neurons were transfected with a construct encoding A53T α-synuclein or an empty vector control. A53T α-synuclein-transfected cells were subsequently treated with a titration of the reference non-selective SCD inhibitor CAY10566 or DMSO as a vehicle control. Analysis of cumulative single cell survival data indicated that relative to DMSO controls, the risk of neuron death was significantly reduced by treatment with CAY10566 at all tested concentrations in the A53T α-synuclein neurons (FIG. 17 and Table 1). Within the relatively narrow 10-fold concentration range tested (3 μM to 0.3 μM), there was no indication of a concentration-dependent effect. This may indicate a saturation of the maximal protective effect at the tested concentrations, or that higher doses are overall less well tolerated by the cells, so any enhanced protection could be obscured by general toxicity.

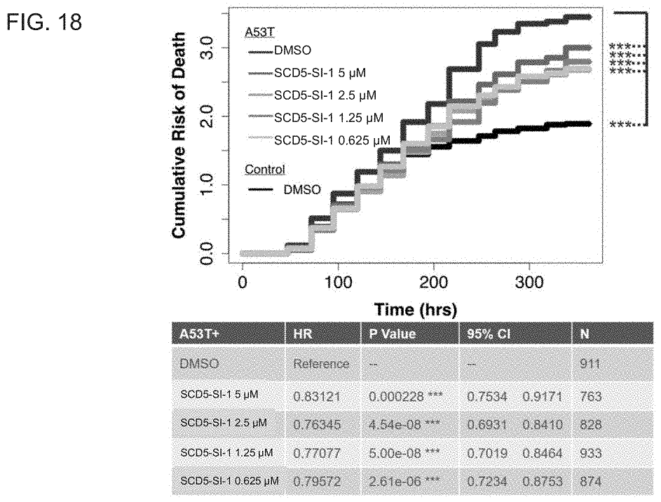

To better understand the relative contributions of different SCD isoforms in promoting protection against A53T α-synuclein toxicity, tool compounds were developed that exhibited an SCD5-selective inhibitor profile. Compounds with this selectivity profile have not been previously described in the literature. SCD5 Selective Inhibitor 1 (SCD5-SI-1) is a SCD5-selective compound that exhibits sub-micromolar potency in yeast growth inhibition assays, and was selected for further study in mammalian cells. Human iPSC-derived neurons were transfected with a construct encoding A53T α-synuclein or an empty vector control. A53T α-synuclein transfected cells were subsequently treated with a titration of the SCD5-selective inhibitor SCD5 Selective Inhibitor 1 or DMSO as a vehicle control. Analysis of cumulative single cell survival data indicated that relative to DMSO controls, the risk of neuron death was significantly reduced by treatment with SCD5 Selective Inhibitor 1 at all tested concentrations in the A53T α-synuclein neurons (FIG. 18). Within the relatively narrow 10-fold concentration range tested (5 μM to 0.6 μM), there was no indication of a concentration-dependent effect. This may indicate a saturation of the maximal protective effect at the tested concentrations, or that higher doses are overall less well tolerated by the cells, so any enhanced protection could be obscured by general toxicity.

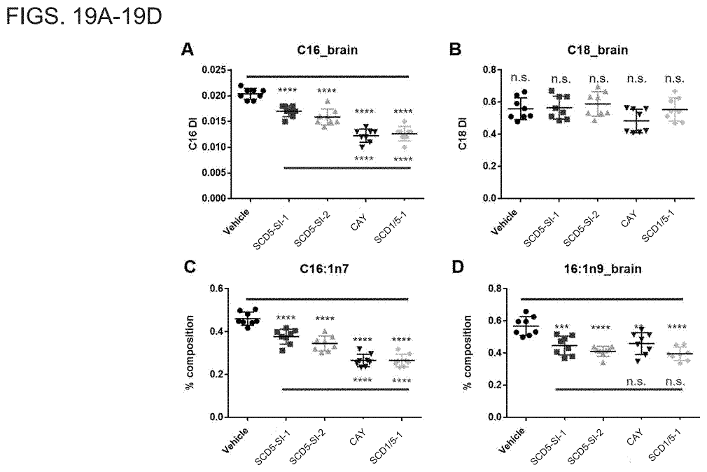

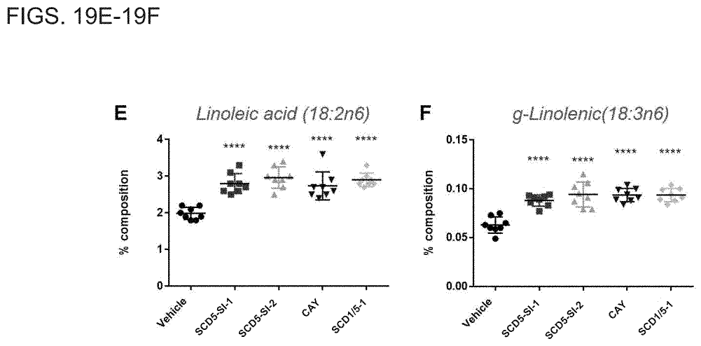

To identify potential central nervous system (CNS) pharmacodynamic markers that respond to inhibition of SCD, guinea pigs were selected as a model organism. Unlike rats and mice, guinea pigs express an SCD isoform similar to human SCD5, and expression of this isoform is enriched in the brain. For these reasons, this species was selected for evaluating both SCD5-selective and non-selective inhibitors. Potential effects of SCD inhibitors on steady state brain fatty acid saturation state, as well as all fatty acid levels, were evaluated by dosing guinea pigs orally twice a day for 5 days with either vehicle, SCD5-selective compounds (SCD5 Selective Inhibitor 1 or SCD5 Selective Inhibitor 2), or non-selective SCD inhibitors (CAY10566 or SCD1/SCD5 Inhibitor 1 (“SCD1/5-1”)). SCD5 Selective Inhibitor 1 is a SCD5-selective compound with >3000-fold selectivity over SCD1 that exhibits sub-micromolar potency in yeast growth inhibition assays. SCD5 Selective Inhibitor 2 is a SCD5-selective compound with >500-fold selectivity over SCD1 that exhibits sub-micromolar potency in yeast growth inhibition assays. SCD1/SCD5 Inhibitor 1 approximately equivalent potency towards SCD1 and SCD5 that exhibits sub-micromolar potency in yeast growth inhibition assays. All compounds were evaluated at 25 mg/kg. On the last day of the study, the brains from these guinea pigs were harvested and evaluated for changes in fatty acid levels and saturation status. The desaturation index (DI) was calculated for 16 and 18 carbon chain fatty acids (C16 and C18 respectively) by taking the ratio of desaturated to saturated fatty acid of each species. Relative to vehicle, all compounds significantly reduced the C16 DI (FIG. 19A). No significant effects were observed on the C18 DI (FIG. 19B). The relative levels of individual monounsaturated C16 fatty acids (expressed as the % composition of total) was also evaluated. For both positional isomers of monounsaturated C16 fatty acids, C16:1 n7 and C16:1 n9, inhibitors of both SCD1/SCD5 selectivity profiles significantly reduced monounsaturated fatty acid levels (FIGS. 19C and 19D). The data in FIGS. 19A-19D are consistent with compounds having SCD inhibitory activity, in which there is a decrease in the levels of unsaturated fatty acids. The C16:1 n9 fatty acid is derived from C18:1 n9 through beta-oxidation. Thus, a decrease in this fatty acid indicated that although no effects were observed in the overall C18 DI, there was a reduction in the monounsaturated C18 species. Interestingly, probing brain samples for the relative levels of linoleic acid (18:2n6) (FIG. 19E) and gamma-linoleic acid (18:3n6) (FIG. 19F) revealed that levels of these essential omega-6 fatty acids both significantly increased with administration of SCD5-selective or non-selective compounds. This inverse relationship in changes to mono- and poly-unsaturated fatty acid levels is consistent with reports in the literature. These data all indicate that both a decrease in desaturated fatty acids by selective inhibition of SCD5, as well as inhibition of both SCD isoforms, result in a measurable pharmacodynamic response in the tissue of interest for CNS indications.

### OTHER EMBODIMENTS

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

Other embodiments are in the claims.

1. A method of treating a neurological disorder in a subject in need thereof, the method comprising administering a fatty acid synthase (FASN) inhibitor in an amount sufficient to suppress toxicity in a cell related to protein misfolding and/or aggregation.

2. A method of suppressing toxicity in a cell related to protein misfolding and/or aggregation in a subject, the method comprising contacting a cell with a FASN inhibitor.

3. The method of claim 1, wherein the toxicity in the cell is related to protein aggregation related to misfolding of a protein.

4. The method of claim 1, wherein the toxicity in the cell is related to misfolding and/or aggregation of α-synuclein or ApoE4.

5-6. (canceled)

7. A method of treating a neurological disorder in a subject in need thereof, the method comprising:
(a) determining the expression level of α-synuclein, ApoE4, or an undesired form thereof in the subject;
(b) administering an effective amount of a FASN inhibitor to the subject if the level of α-synuclein, ApoE4, and/or the undesired form thereof is greater than a predetermined level.

8. A method of treating a neurological disease in a subject in need thereof, wherein the subject has an elevated level, or is predicted to have an elevated level of α-synuclein, ApoE4, or an undesired form thereof the method comprising administering an effective amount of a FASN inhibitor to the subject.

9. The method of claim 8, wherein the subject is predicted to have an elevated level of α-synuclein, ApoE4, and/or an undesired form thereof based on genetic markers.

10. The method of claim 1, wherein the subject carries one or two copies of the ApoE4 allele.

11. (canceled)

12. The method of claim 1, wherein the neurological disorder is Alzheimer's disease (AD), mild cognitive impairment (MCI), cerebral amyloid angiopathy (CAA), dementia associated with Down syndrome, Parkinson's disease (PD), dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, Down's syndrome, multiple sclerosis.

13-15. (canceled)

16. The method of claim 1, wherein the neurological disorder is Parkinson's disease (PD), dementia with Lewy bodies, pure autonomic failure, multiple system atrophy, incidental Lewy body disease, pantothenate kinase-associated neurodegeneration, Gaucher disease, or the Parkinsonism-dementia complex of Guam.

17. The method of claim 16, wherein the neurological disorder does not comprise a PINK1 mutation.

18. (canceled)

19. The method of claim 1, wherein the neurological disorder is AD, Alexander disease, amyotrophic lateral sclerosis (ALS), a prion disease, Huntington's disease, Machado-Joseph disease, Pick's disease, or frontotemporal dementia.

20. The method of claim 19, wherein the prion disease is Creutzfeldt-Jakob disease.

21. The method of claim 1, wherein the neurological disorder is a neurodegenerative disorder.

22. The method of claim 21, wherein the neurodegenerative disorder is Alpers' disease, ataxia telangectsia, Canavan disease, Cockayne syndrome, corticobasal degeneration, Kennedy's disease, Krabbe disease, Pelizaeus-Merzbacher disease, primary lateral sclerosis, Refsum's disease, Sandhoff disease, Schilder's disease, Steele-Richardson-Olszewski disease, tabes dorsalis, vascular dementia, or Guillain-Barre Syndrome.

23. The method of claim 1, wherein the neurological disorder is an ApoE-associated neurodegenerative disorder.

24. The method of claim 23, wherein the ApoE-associated neurodegenerative disorder is AD, vascular cognitive impairment, cerebral amyloid angiopathy, traumatic brain injury, or multiple sclerosis.

25. The method of claim 24, wherein the ApoE-associated disorder is AD.

26-28. (canceled)
